SEQUENCE LISTING (1603 sequences)

SEQ ID NO: 1 - RNA, 45 nt, Artificial Sequence
  Description of Artificial Sequence Synthetic oligonucleotide
guggaauaguauaacaauaugcuaaauguuguuauaguaucccac

SEQ ID NO: 2 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaucucugaacuaaucauc

SEQ ID NO: 3 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaucucugaacuaaucauc

SEQ ID NO: 4 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauaucucugaacuaaucauc

SEQ ID NO: 5 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaucucugaacuaaucauc

SEQ ID NO: 6 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauaucucugaacuaaucauc

SEQ ID NO: 7 - RNA, 100 nt, Artificial Sequence
  Description of Artificial Sequence Synthetic polynucleotide
gauauugugacauaucucugcacucaucacacaaaagaugcaaaucuucuauaguacauucagggagggggcauuuuaacauaucucugaacuaaucauc

SEQ ID NO: 8 - RNA, 100 nt, Artificial Sequence
  Description of Artificial Sequence Synthetic polynucleotide
gauauugugacauaucucugcacucaucacacaaaagaugcaaaucuucuauagacaggucagggagggggcauuucaauauaucucugaacuaaucauc

SEQ ID NO: 9 - RNA, 100 nt, Artificial Sequence
  Description of Artificial Sequence Synthetic polynucleotide
gauauugugacauaucucugcacucaucacacaaaagaugcaaaucuucuauagcacaaucagggagggggcauucuaauauaucucugaacuaaucauc

SEQ ID NO: 10 - RNA, 100 nt, Artificial Sequence
  Description of Artificial Sequence Synthetic polynucleotide
gauauugugacauaucucugcacucaucacacaaaagaugcaaaucuucuauaguuagaucagggagggggcaucuuaauauaucucugaacuaaucauc

SEQ ID NO: 11 - RNA, 100 nt, Artificial Sequence
  Description of Artificial Sequence Synthetic polynucleotide
gauauugugacauaucucugcacucaucacacaaaagaugcaaaucuucuauagaaaauucagggagggggcauuuuaauauaucucugaacuaaucauc

SEQ ID NO: 12 - RNA, 100 nt, Artificial Sequence
  Description of Artificial Sequence Synthetic polynucleotide
gauauugugacauaucucugcacucaucacacaaaagaugcaaaucuucuauaaacauaacagggagggggcauuuuaacauaucucugaacuaaucauc

SEQ ID NO: 13 - RNA, 100 nt, Artificial Sequence
  Description of Artificial Sequence Synthetic polynucleotide
gauauugugacauaucucugcacucaucacacaaaagaugcaaaucuucuauauaaguaacagggagggggcauuucaauauaucucugaacuaaucauc

SEQ ID NO: 14 - RNA, 100 nt, Artificial Sequence
  Description of Artificial Sequence Synthetic polynucleotide
gauauugugacauaucucugcacucaucacacaaaagaugcaaaucuucuauaucgcggacagggagggggcaucuuaauauaucucugaacuaaucauc

SEQ ID NO: 15 - RNA, 100 nt, Artificial Sequence
  Description of Artificial Sequence Synthetic polynucleotide
gauauugugacauaucucugcacucaucacacaaaagaugcaaaucuucuauaccccaaacagggagggggcauuuuaauauaucucugaacuaaucauc

SEQ ID NO: 16 - RNA, 100 nt, Artificial Sequence
  Description of Artificial Sequence Synthetic polynucleotide
gauauugugacauaucucugcacucaucacacaaaagaugcaaaucuucuauugucgacacagggagggggcauuuuaacauaucucugaacuaaucauc

SEQ ID NO: 17 - RNA, 100 nt, Artificial Sequence
  Description of Artificial Sequence Synthetic polynucleotide
gauauugugacauaucucugcacucaucacacaaaagaugcaaaucuucuauccccgacacagggagggggcauuucaauauaucucugaacuaaucauc

SEQ ID NO: 18 - RNA, 100 nt, Artificial Sequence
  Description of Artificial Sequence Synthetic polynucleotide
gauauugugacauaucucugcacucaucacacaaaagaugcaaaucuucuauuucuaacacagggagggggcauucuaauauaucucugaacuaaucauc

SEQ ID NO: 19 - RNA, 100 nt, Artificial Sequence
  Description of Artificial Sequence Synthetic polynucleotide
gauauugugacauaucucugcacucaucacacaaaagaugcaaaucuucuaucaccgacacagggagggggcaucuuaauauaucucugaacuaaucauc

SEQ ID NO: 20 - RNA, 100 nt, Artificial Sequence
  Description of Artificial Sequence Synthetic polynucleotide
gauauugugacauaucucugcacucaucacacaaaagaugcaaaucuucuauuacuaacacagggagggggcauuuuaauauaucucugaacuaaucauc

SEQ ID NO: 21 - RNA, 100 nt, Artificial Sequence
  Description of Artificial Sequence Synthetic polynucleotide
gauauugugacauaucucugcacucaucacacaaaagaugcaaaucuucuagaucucccacagggagggggcauuuuaacauaucucugaacuaaucauc

SEQ ID NO: 22 - RNA, 100 nt, Artificial Sequence
  Description of Artificial Sequence Synthetic polynucleotide
gauauugugacauaucucugcacucaucacacaaaagaugcaaaucuucuagucacgccacagggagggggcauuucaauauaucucugaacuaaucauc

SEQ ID NO: 23 - RNA, 100 nt, Artificial Sequence
  Description of Artificial Sequence Synthetic polynucleotide
gauauugugacauaucucugcacucaucacacaaaagaugcaaaucuucuaauauccccacagggagggggcauucuaauauaucucugaacuaaucauc

SEQ ID NO: 24 - RNA, 100 nt, Artificial Sequence
  Description of Artificial Sequence Synthetic polynucleotide
gauauugugacauaucucugcacucaucacacaaaagaugcaaaucuucuacguccgccacagggagggggcaucuuaauauaucucugaacuaaucauc

SEQ ID NO: 25 - RNA, 100 nt, Artificial Sequence
  Description of Artificial Sequence Synthetic polynucleotide
gauauugugacauaucucugcacucaucacacaaaagaugcaaaucuucuagaaucgccacagggagggggcauuuuaauauaucucugaacuaaucauc

SEQ ID NO: 26 - RNA, 100 nt, Artificial Sequence
  Description of Artificial Sequence Synthetic polynucleotide
gauauugugacauaucucugcacucaucacacaaaagaugcaaaucuucuuacaguuccacagggagggggcauuuuaacauaucucugaacuaaucauc

SEQ ID NO: 27 - RNA, 100 nt, Artificial Sequence
  Description of Artificial Sequence Synthetic polynucleotide
gauauugugacauaucucugcacucaucacacaaaagaugcaaaucuucuucuacuuccacagggagggggcauuucaauauaucucugaacuaaucauc

SEQ ID NO: 28 - RNA, 100 nt, Artificial Sequence
  Description of Artificial Sequence Synthetic polynucleotide
gauauugugacauaucucugcacucaucacacaaaagaugcaaaucuucuucgcguuccacagggagggggcauucuaauauaucucugaacuaaucauc

SEQ ID NO: 29 - RNA, 100 nt, Artificial Sequence
  Description of Artificial Sequence Synthetic polynucleotide
gauauugugacauaucucugcacucaucacacaaaagaugcaaaucuucuccguauuccacagggagggggcaucuuaauauaucucugaacuaaucauc

SEQ ID NO: 30 - RNA, 100 nt, Artificial Sequence
  Description of Artificial Sequence Synthetic polynucleotide
gauauugugacauaucucugcacucaucacacaaaagaugcaaaucuucuugucucuccacagggagggggcauuuuaauauaucucugaacuaaucauc

SEQ ID NO: 31 - RNA, 100 nt, Artificial Sequence
  Description of Artificial Sequence Synthetic polynucleotide
gauauugugacauaucucugcacucaucacacaaaagaugcaaaucuucaccgacauccacagggagggggcauuuuaacauaucucugaacuaaucauc

SEQ ID NO: 32 - RNA, 100 nt, Artificial Sequence
  Description of Artificial Sequence Synthetic polynucleotide
gauauugugacauaucucugcacucaucacacaaaagaugcaaaucuucccauuuauccacagggagggggcauuucaauauaucucugaacuaaucauc

SEQ ID NO: 33 - RNA, 100 nt, Artificial Sequence
  Description of Artificial Sequence Synthetic polynucleotide
gauauugugacauaucucugcacucaucacacaaaagaugcaaaucuucccuuuaauccacagggagggggcauucuaauauaucucugaacuaaucauc

SEQ ID NO: 34 - RNA, 100 nt, Artificial Sequence
  Description of Artificial Sequence Synthetic polynucleotide
gauauugugacauaucucugcacucaucacacaaaagaugcaaaucuucaucguuauccacagggagggggcaucuuaauauaucucugaacuaaucauc

SEQ ID NO: 35 - RNA, 100 nt, Artificial Sequence
  Description of Artificial Sequence Synthetic polynucleotide
gauauugugacauaucucugcacucaucacacaaaagaugcaaaucuucaagaaaauccacagggagggggcauuuuaauauaucucugaacuaaucauc

SEQ ID NO: 36 - RNA, 100 nt, Artificial Sequence
  Description of Artificial Sequence Synthetic polynucleotide
gauauugugacauaucucugcacucaucacacaaaagaugcaaaucuuaaugacgauccacagggagggggcauuuuaacauaucucugaacuaaucauc

SEQ ID NO: 37 - RNA, 100 nt, Artificial Sequence
  Description of Artificial Sequence Synthetic polynucleotide
gauauugugacauaucucugcacucaucacacaaaagaugcaaaucuugggccagauccacagggagggggcauuucaauauaucucugaacuaaucauc

SEQ ID NO: 38 - RNA, 100 nt, Artificial Sequence
  Description of Artificial Sequence Synthetic polynucleotide
gauauugugacauaucucugcacucaucacacaaaagaugcaaaucuuagugucgauccacagggagggggcauucuaauauaucucugaacuaaucauc

SEQ ID NO: 39 - RNA, 100 nt, Artificial Sequence
  Description of Artificial Sequence Synthetic polynucleotide
gauauugugacauaucucugcacucaucacacaaaagaugcaaaucuuagcuuugauccacagggagggggcaucuuaauauaucucugaacuaaucauc

SEQ ID NO: 40 - RNA, 100 nt, Artificial Sequence
  Description of Artificial Sequence Synthetic polynucleotide
gauauugugacauaucucugcacucaucacacaaaagaugcaaaucuuggaauugauccacagggagggggcauuuuaauauaucucugaacuaaucauc

SEQ ID NO: 41 - RNA, 100 nt, Artificial Sequence
  Description of Artificial Sequence Synthetic polynucleotide
gauauugugacauaucucugcacucaucacacaaaagaugcaaaucucagucuggauccacagggagggggcauuuuaacauaucucugaacuaaucauc

SEQ ID NO: 42 - RNA, 100 nt, Artificial Sequence
  Description of Artificial Sequence Synthetic polynucleotide
gauauugugacauaucucugcacucaucacacaaaagaugcaaaucuaaccauggauccacagggagggggcauuucaauauaucucugaacuaaucauc

SEQ ID NO: 43 - RNA, 100 nt, Artificial Sequence
  Description of Artificial Sequence Synthetic polynucleotide
gauauugugacauaucucugcacucaucacacaaaagaugcaaaucuccaccuggauccacagggagggggcauucuaauauaucucugaacuaaucauc

SEQ ID NO: 44 - RNA, 100 nt, Artificial Sequence
  Description of Artificial Sequence Synthetic polynucleotide
gauauugugacauaucucugcacucaucacacaaaagaugcaaaucugugcacggauccacagggagggggcaucuuaauauaucucugaacuaaucauc

SEQ ID NO: 45 - RNA, 100 nt, Artificial Sequence
  Description of Artificial Sequence Synthetic polynucleotide
gauauugugacauaucucugcacucaucacacaaaagaugcaaaucugacaucggauccacagggagggggcauuuuaauauaucucugaacuaaucauc

SEQ ID NO: 46 - RNA, 100 nt, Artificial Sequence
  Description of Artificial Sequence Synthetic polynucleotide
gauauugugacauaucucugcacucaucacacaaaagaugcaaauccagucaaggauccacagggagggggcauuuuaacauaucucugaacuaaucauc

SEQ ID NO: 47 - RNA, 100 nt, Artificial Sequence
  Description of Artificial Sequence Synthetic polynucleotide
gauauugugacauaucucugcacucaucacacaaaagaugcaaaucacuccgaggauccacagggagggggcauuucaauauaucucugaacuaaucauc

SEQ ID NO: 48 - RNA, 100 nt, Artificial Sequence
  Description of Artificial Sequence Synthetic polynucleotide
gauauugugacauaucucugcacucaucacacaaaagaugcaaauccgaaacaggauccacagggagggggcauucuaauauaucucugaacuaaucauc

SEQ ID NO: 49 - RNA, 100 nt, Artificial Sequence
  Description of Artificial Sequence Synthetic polynucleotide
gauauugugacauaucucugcacucaucacacaaaagaugcaaaucauacgaaggauccacagggagggggcaucuuaauauaucucugaacuaaucauc

SEQ ID NO: 50 - RNA, 100 nt, Artificial Sequence
  Description of Artificial Sequence Synthetic polynucleotide
gauauugugacauaucucugcacucaucacacaaaagaugcaaaucccggggaggauccacagggagggggcauuuuaauauaucucugaacuaaucauc

SEQ ID NO: 51 - RNA, 100 nt, Artificial Sequence
  Description of Artificial Sequence Synthetic polynucleotide
gauauugugacauaucucugcacucaucacacaaaagaugcaaauauugacuaggauccacagggagggggcauuuuaacauaucucugaacuaaucauc

SEQ ID NO: 52 - RNA, 100 nt, Artificial Sequence
  Description of Artificial Sequence Synthetic polynucleotide
gauauugugacauaucucugcacucaucacacaaaagaugcaaaugaccccuaggauccacagggagggggcauuucaauauaucucugaacuaaucauc

SEQ ID NO: 53 - RNA, 100 nt, Artificial Sequence
  Description of Artificial Sequence Synthetic polynucleotide
gauauugugacauaucucugcacucaucacacaaaagaugcaaaugcgugcuaggauccacagggagggggcauucuaauauaucucugaacuaaucauc

SEQ ID NO: 54 - RNA, 100 nt, Artificial Sequence
  Description of Artificial Sequence Synthetic polynucleotide
gauauugugacauaucucugcacucaucacacaaaagaugcaaaugaggacuaggauccacagggagggggcaucuuaauauaucucugaacuaaucauc

SEQ ID NO: 55 - RNA, 100 nt, Artificial Sequence
  Description of Artificial Sequence Synthetic polynucleotide
gauauugugacauaucucugcacucaucacacaaaagaugcaaaugggaauuaggauccacagggagggggcauuuuaauauaucucugaacuaaucauc

SEQ ID NO: 56 - RNA, 100 nt, Artificial Sequence
  Description of Artificial Sequence Synthetic polynucleotide
gauauugugacauaucucugcacucaucacacaaaagaugcaaaagagagauaggauccacagggagggggcauuuuaacauaucucugaacuaaucauc

SEQ ID NO: 57 - RNA, 100 nt, Artificial Sequence
  Description of Artificial Sequence Synthetic polynucleotide
gauauugugacauaucucugcacucaucacacaaaagaugcaaacgcacgauaggauccacagggagggggcauuucaauauaucucugaacuaaucauc

SEQ ID NO: 58 - RNA, 100 nt, Artificial Sequence
  Description of Artificial Sequence Synthetic polynucleotide
gauauugugacauaucucugcacucaucacacaaaagaugcaaaccagagauaggauccacagggagggggcauucuaauauaucucugaacuaaucauc

SEQ ID NO: 59 - RNA, 100 nt, Artificial Sequence
  Description of Artificial Sequence Synthetic polynucleotide
gauauugugacauaucucugcacucaucacacaaaagaugcaaaaggcgaauaggauccacagggagggggcaucuuaauauaucucugaacuaaucauc

SEQ ID NO: 60 - RNA, 100 nt, Artificial Sequence
  Description of Artificial Sequence Synthetic polynucleotide
gauauugugacauaucucugcacucaucacacaaaagaugcaaagcaagaauaggauccacagggagggggcauuuuaauauaucucugaacuaaucauc

SEQ ID NO: 61 - RNA, 100 nt, Artificial Sequence
  Description of Artificial Sequence Synthetic polynucleotide
gauauugugacauaucucugcacucaucacacaaaagaugcaaccccgauauaggauccacagggagggggcauuuuaacauaucucugaacuaaucauc

SEQ ID NO: 62 - RNA, 100 nt, Artificial Sequence
  Description of Artificial Sequence Synthetic polynucleotide
gauauugugacauaucucugcacucaucacacaaaagaugcaacuaaaauauaggauccacagggagggggcauuucaauauaucucugaacuaaucauc

SEQ ID NO: 63 - RNA, 100 nt, Artificial Sequence
  Description of Artificial Sequence Synthetic polynucleotide
gauauugugacauaucucugcacucaucacacaaaagaugcaacguggguauaggauccacagggagggggcauucuaauauaucucugaacuaaucauc

SEQ ID NO: 64 - RNA, 100 nt, Artificial Sequence
  Description of Artificial Sequence Synthetic polynucleotide
gauauugugacauaucucugcacucaucacacaaaagaugcaacugggauauaggauccacagggagggggcaucuuaauauaucucugaacuaaucauc

SEQ ID NO: 65 - RNA, 100 nt, Artificial Sequence
  Description of Artificial Sequence Synthetic polynucleotide
gauauugugacauaucucugcacucaucacacaaaagaugcaauggagauauaggauccacagggagggggcauuuuaauauaucucugaacuaaucauc

SEQ ID NO: 66 - RNA, 100 nt, Artificial Sequence
  Description of Artificial Sequence Synthetic polynucleotide
gauauugugacauaucucugcacucaucacacaaaagaugcauccggacuauaggauccacagggagggggcauuuuaacauaucucugaacuaaucauc

SEQ ID NO: 67 - RNA, 100 nt, Artificial Sequence
  Description of Artificial Sequence Synthetic polynucleotide
gauauugugacauaucucugcacucaucacacaaaagaugcauccuggcuauaggauccacagggagggggcauuucaauauaucucugaacuaaucauc

SEQ ID NO: 68 - RNA, 100 nt, Artificial Sequence
  Description of Artificial Sequence Synthetic polynucleotide
gauauugugacauaucucugcacucaucacacaaaagaugcaccuagacuauaggauccacagggagggggcauucuaauauaucucugaacuaaucauc

SEQ ID NO: 69 - RNA, 100 nt, Artificial Sequence
  Description of Artificial Sequence Synthetic polynucleotide
gauauugugacauaucucugcacucaucacacaaaagaugcacccggacuauaggauccacagggagggggcaucuuaauauaucucugaacuaaucauc

SEQ ID NO: 70 - RNA, 100 nt, Artificial Sequence
  Description of Artificial Sequence Synthetic polynucleotide
gauauugugacauaucucugcacucaucacacaaaagaugcacugaggcuauaggauccacagggagggggcauuuuaauauaucucugaacuaaucauc

SEQ ID NO: 71 - RNA, 100 nt, Artificial Sequence
  Description of Artificial Sequence Synthetic polynucleotide
gauauugugacauaucucugcacucaucacacaaaagaugcuugagaucuauaggauccacagggagggggcauuuuaacauaucucugaacuaaucauc

SEQ ID NO: 72 - RNA, 100 nt, Artificial Sequence
  Description of Artificial Sequence Synthetic polynucleotide
gauauugugacauaucucugcacucaucacacaaaagaugcuugggaucuauaggauccacagggagggggcauuucaauauaucucugaacuaaucauc

SEQ ID NO: 73 - RNA, 100 nt, Artificial Sequence
  Description of Artificial Sequence Synthetic polynucleotide
gauauugugacauaucucugcacucaucacacaaaagaugcuuugcgucuauaggauccacagggagggggcauucuaauauaucucugaacuaaucauc

SEQ ID NO: 74 - RNA, 100 nt, Artificial Sequence
  Description of Artificial Sequence Synthetic polynucleotide
gauauugugacauaucucugcacucaucacacaaaagaugccugcacucuauaggauccacagggagggggcaucuuaauauaucucugaacuaaucauc

SEQ ID NO: 75 - RNA, 100 nt, Artificial Sequence
  Description of Artificial Sequence Synthetic polynucleotide
gauauugugacauaucucugcacucaucacacaaaagaugccgcgggucuauaggauccacagggagggggcauuuuaauauaucucugaacuaaucauc

SEQ ID NO: 76 - RNA, 100 nt, Artificial Sequence
  Description of Artificial Sequence Synthetic polynucleotide
gauauugugacauaucucugcacucaucacacaaaagaugguuuaauucuauaggauccacagggagggggcauuucaauauaucucugaacuaaucauc

SEQ ID NO: 77 - RNA, 100 nt, Artificial Sequence
  Description of Artificial Sequence Synthetic polynucleotide
gauauugugacauaucucugcacucaucacacaaaagauggcuggguucuauaggauccacagggagggggcauucuaauauaucucugaacuaaucauc

SEQ ID NO: 78 - RNA, 100 nt, Artificial Sequence
  Description of Artificial Sequence Synthetic polynucleotide
gauauugugacauaucucugcacucaucacacaaaagaugacuccguucuauaggauccacagggagggggcaucuuaauauaucucugaacuaaucauc

SEQ ID NO: 79 - RNA, 100 nt, Artificial Sequence
  Description of Artificial Sequence Synthetic polynucleotide
gauauugugacauaucucugcacucaucacacaaaagaugguuacauucuauaggauccacagggagggggcauuuuaauauaucucugaacuaaucauc

SEQ ID NO: 80 - RNA, 100 nt, Artificial Sequence
  Description of Artificial Sequence Synthetic polynucleotide
gauauugugacauaucucugcacucaucacacaaaagaucugucacuucuauaggauccacagggagggggcauuuuaacauaucucugaacuaaucauc

SEQ ID NO: 81 - RNA, 100 nt, Artificial Sequence
  Description of Artificial Sequence Synthetic polynucleotide
gauauugugacauaucucugcacucaucacacaaaagaucuugagcuucuauaggauccacagggagggggcauuucaauauaucucugaacuaaucauc

SEQ ID NO: 82 - RNA, 100 nt, Artificial Sequence
  Description of Artificial Sequence Synthetic polynucleotide
gauauugugacauaucucugcacucaucacacaaaagaucuuaggcuucuauaggauccacagggagggggcauucuaauauaucucugaacuaaucauc

SEQ ID NO: 83 - RNA, 100 nt, Artificial Sequence
  Description of Artificial Sequence Synthetic polynucleotide
gauauugugacauaucucugcacucaucacacaaaagauaagugccuucuauaggauccacagggagggggcaucuuaauauaucucugaacuaaucauc

SEQ ID NO: 84 - RNA, 100 nt, Artificial Sequence
  Description of Artificial Sequence Synthetic polynucleotide
gauauugugacauaucucugcacucaucacacaaaagaucuuccacuucuauaggauccacagggagggggcauuuuaauauaucucugaacuaaucauc

SEQ ID NO: 85 - RNA, 100 nt, Artificial Sequence
  Description of Artificial Sequence Synthetic polynucleotide
gauauugugacauaucucugcacucaucacacaaaagacaacuuucuucuauaggauccacagggagggggcauuuuaacauaucucugaacuaaucauc

SEQ ID NO: 86 - RNA, 100 nt, Artificial Sequence
  Description of Artificial Sequence Synthetic polynucleotide
gauauugugacauaucucugcacucaucacacaaaagaccacucucuucuauaggauccacagggagggggcauuucaauauaucucugaacuaaucauc

SEQ ID NO: 87 - RNA, 100 nt, Artificial Sequence
  Description of Artificial Sequence Synthetic polynucleotide
gauauugugacauaucucugcacucaucacacaaaagaacgguuucuucuauaggauccacagggagggggcauucuaauauaucucugaacuaaucauc

SEQ ID NO: 88 - RNA, 100 nt, Artificial Sequence
  Description of Artificial Sequence Synthetic polynucleotide
gauauugugacauaucucugcacucaucacacaaaagacaccucucuucuauaggauccacagggagggggcaucuuaauauaucucugaacuaaucauc

SEQ ID NO: 89 - RNA, 100 nt, Artificial Sequence
  Description of Artificial Sequence Synthetic polynucleotide
gauauugugacauaucucugcacucaucacacaaaagacuaguuucuucuauaggauccacagggagggggcauuuuaauauaucucugaacuaaucauc

SEQ ID NO: 90 - RNA, 100 nt, Artificial Sequence
  Description of Artificial Sequence Synthetic polynucleotide
gauauugugacauaucucugcacucaucacacaaaagcgauccaucuucuauaggauccacagggagggggcauuuuaacauaucucugaacuaaucauc

SEQ ID NO: 91 - RNA, 100 nt, Artificial Sequence
  Description of Artificial Sequence Synthetic polynucleotide
gauauugugacauaucucugcacucaucacacaaaagcacgucaucuucuauaggauccacagggagggggcauuucaauauaucucugaacuaaucauc

SEQ ID NO: 92 - RNA, 100 nt, Artificial Sequence
  Description of Artificial Sequence Synthetic polynucleotide
gauauugugacauaucucugcacucaucacacaaaagcuaguuaucuucuauaggauccacagggagggggcauucuaauauaucucugaacuaaucauc

SEQ ID NO: 93 - RNA, 100 nt, Artificial Sequence
  Description of Artificial Sequence Synthetic polynucleotide
gauauugugacauaucucugcacucaucacacaaaagcguuuuaucuucuauaggauccacagggagggggcaucuuaauauaucucugaacuaaucauc

SEQ ID NO: 94 - RNA, 100 nt, Artificial Sequence
  Description of Artificial Sequence Synthetic polynucleotide
gauauugugacauaucucugcacucaucacacaaaagucugauaucuucuauaggauccacagggagggggcauuuuaauauaucucugaacuaaucauc

SEQ ID NO: 95 - RNA, 100 nt, Artificial Sequence
  Description of Artificial Sequence Synthetic polynucleotide
gauauugugacauaucucugcacucaucacacaaaacuuauuaaucuucuauaggauccacagggagggggcauuuuaacauaucucugaacuaaucauc

SEQ ID NO: 96 - RNA, 100 nt, Artificial Sequence
  Description of Artificial Sequence Synthetic polynucleotide
gauauugugacauaucucugcacucaucacacaaaauuuaucaaucuucuauaggauccacagggagggggcauuucaauauaucucugaacuaaucauc

SEQ ID NO: 97 - RNA, 100 nt, Artificial Sequence
  Description of Artificial Sequence Synthetic polynucleotide
gauauugugacauaucucugcacucaucacacaaaauuaaacaaucuucuauaggauccacagggagggggcauucuaauauaucucugaacuaaucauc

SEQ ID NO: 98 - RNA, 100 nt, Artificial Sequence
  Description of Artificial Sequence Synthetic polynucleotide
gauauugugacauaucucugcacucaucacacaaaauuacguaaucuucuauaggauccacagggagggggcauuuuaauauaucucugaacuaaucauc

SEQ ID NO: 99 - RNA, 100 nt, Artificial Sequence
  Description of Artificial Sequence Synthetic polynucleotide
gauauugugacauaucucugcacucaucacacaaaucuauaaaaucuucuauaggauccacagggagggggcauuuuaacauaucucugaacuaaucauc

SEQ ID NO: 100 - RNA, 100 nt, Artificial Sequence
  Description of Artificial Sequence Synthetic polynucleotide
gauauugugacauaucucugcacucaucacacaaauacggaaaaucuucuauaggauccacagggagggggcauuucaauauaucucugaacuaaucauc

SEQ ID NO: 101 - RNA, 100 nt, Artificial Sequence
  Description of Artificial Sequence Synthetic polynucleotide
gauauugugacauaucucugcacucaucacacaaacuccagaaaucuucuauaggauccacagggagggggcauucuaauauaucucugaacuaaucauc

SEQ ID NO: 102 - RNA, 100 nt, Artificial Sequence
  Description of Artificial Sequence Synthetic polynucleotide
gauauugugacauaucucugcacucaucacacaaaucaacaaaaucuucuauaggauccacagggagggggcaucuuaauauaucucugaacuaaucauc

SEQ ID NO: 103 - RNA, 100 nt, Artificial Sequence
  Description of Artificial Sequence Synthetic polynucleotide
gauauugugacauaucucugcacucaucacacaaacauacgaaaucuucuauaggauccacagggagggggcauuuuaauauaucucugaacuaaucauc

SEQ ID NO: 104 - RNA, 100 nt, Artificial Sequence
  Description of Artificial Sequence Synthetic polynucleotide
gauauugugacauaucucugcacucaucacacaauaucgucaaaucuucuauaggauccacagggagggggcauuuuaacauaucucugaacuaaucauc

SEQ ID NO: 105 - RNA, 100 nt, Artificial Sequence
  Description of Artificial Sequence Synthetic polynucleotide
gauauugugacauaucucugcacucaucacacaauccgcacaaaucuucuauaggauccacagggagggggcauuucaauauaucucugaacuaaucauc

SEQ ID NO: 106 - RNA, 100 nt, Artificial Sequence
  Description of Artificial Sequence Synthetic polynucleotide
gauauugugacauaucucugcacucaucacacaacuugaccaaaucuucuauaggauccacagggagggggcauucuaauauaucucugaacuaaucauc

SEQ ID NO: 107 - RNA, 100 nt, Artificial Sequence
  Description of Artificial Sequence Synthetic polynucleotide
gauauugugacauaucucugcacucaucacacaaucuuuccaaaucuucuauaggauccacagggagggggcaucuuaauauaucucugaacuaaucauc

SEQ ID NO: 108 - RNA, 100 nt, Artificial Sequence
  Description of Artificial Sequence Synthetic polynucleotide
gauauugugacauaucucugcacucaucacacaacgcgaccaaaucuucuauaggauccacagggagggggcauuuuaauauaucucugaacuaaucauc

SEQ ID NO: 109 - RNA, 100 nt, Artificial Sequence
  Description of Artificial Sequence Synthetic polynucleotide
gauauugugacauaucucugcacucaucacacaucuauggcaaaucuucuauaggauccacagggagggggcauuuuaacauaucucugaacuaaucauc

SEQ ID NO: 110 - RNA, 100 nt, Artificial Sequence
  Description of Artificial Sequence Synthetic polynucleotide
gauauugugacauaucucugcacucaucacacacgcgccgcaaaucuucuauaggauccacagggagggggcauuucaauauaucucugaacuaaucauc

SEQ ID NO: 111 - RNA, 100 nt, Artificial Sequence
  Description of Artificial Sequence Synthetic polynucleotide
gauauugugacauaucucugcacucaucacacauguaucgcaaaucuucuauaggauccacagggagggggcauucuaauauaucucugaacuaaucauc

SEQ ID NO: 112 - RNA, 100 nt, Artificial Sequence
  Description of Artificial Sequence Synthetic polynucleotide
gauauugugacauaucucugcacucaucacacauugcucgcaaaucuucuauaggauccacagggagggggcaucuuaauauaucucugaacuaaucauc

SEQ ID NO: 113 - RNA, 100 nt, Artificial Sequence
  Description of Artificial Sequence Synthetic polynucleotide
gauauugugacauaucucugcacucaucacacaccgcccgcaaaucuucuauaggauccacagggagggggcauuuuaauauaucucugaacuaaucauc

SEQ ID NO: 114 - RNA, 100 nt, Artificial Sequence
  Description of Artificial Sequence Synthetic polynucleotide
gauauugugacauaucucugcacucaucacacuucuucugcaaaucuucuauaggauccacagggagggggcauuuuaacauaucucugaacuaaucauc

SEQ ID NO: 115 - RNA, 100 nt, Artificial Sequence
  Description of Artificial Sequence Synthetic polynucleotide
gauauugugacauaucucugcacucaucacaccugcucugcaaaucuucuauaggauccacagggagggggcauuucaauauaucucugaacuaaucauc

SEQ ID NO: 116 - RNA, 100 nt, Artificial Sequence
  Description of Artificial Sequence Synthetic polynucleotide
gauauugugacauaucucugcacucaucacaccuuguuugcaaaucuucuauaggauccacagggagggggcauucuaauauaucucugaacuaaucauc

SEQ ID NO: 117 - RNA, 100 nt, Artificial Sequence
  Description of Artificial Sequence Synthetic polynucleotide
gauauugugacauaucucugcacucaucacaccuauauugcaaaucuucuauaggauccacagggagggggcauuuuaauauaucucugaacuaaucauc

SEQ ID NO: 118 - RNA, 100 nt, Artificial Sequence
  Description of Artificial Sequence Synthetic polynucleotide
gauauugugacauaucucugcacucaucacaguuguaaugcaaaucuucuauaggauccacagggagggggcauuuuaacauaucucugaacuaaucauc

SEQ ID NO: 119 - RNA, 100 nt, Artificial Sequence
  Description of Artificial Sequence Synthetic polynucleotide
gauauugugacauaucucugcacucaucacaaccugaaugcaaaucuucuauaggauccacagggagggggcauucuaauauaucucugaacuaaucauc

SEQ ID NO: 120 - RNA, 100 nt, Artificial Sequence
  Description of Artificial Sequence Synthetic polynucleotide
gauauugugacauaucucugcacucaucacagcugucaugcaaaucuucuauaggauccacagggagggggcaucuuaauauaucucugaacuaaucauc

SEQ ID NO: 121 - RNA, 100 nt, Artificial Sequence
  Description of Artificial Sequence Synthetic polynucleotide
gauauugugacauaucucugcacucaucacaagaccaaugcaaaucuucuauaggauccacagggagggggcauuuuaauauaucucugaacuaaucauc

SEQ ID NO: 122 - RNA, 100 nt, Artificial Sequence
  Description of Artificial Sequence Synthetic polynucleotide
gauauugugacauaucucugcacucaucacuaccucgaugcaaaucuucuauaggauccacagggagggggcauuuuaacauaucucugaacuaaucauc

SEQ ID NO: 123 - RNA, 100 nt, Artificial Sequence
  Description of Artificial Sequence Synthetic polynucleotide
gauauugugacauaucucugcacucaucaccuuuccgaugcaaaucuucuauaggauccacagggagggggcauuucaauauaucucugaacuaaucauc

SEQ ID NO: 124 - RNA, 100 nt, Artificial Sequence
  Description of Artificial Sequence Synthetic polynucleotide
gauauugugacauaucucugcacucaucacuugccugaugcaaaucuucuauaggauccacagggagggggcauucuaauauaucucugaacuaaucauc

SEQ ID NO: 125 - RNA, 100 nt, Artificial Sequence
  Description of Artificial Sequence Synthetic polynucleotide
gauauugugacauaucucugcacucaucacuguuucgaugcaaaucuucuauaggauccacagggagggggcaucuuaauauaucucugaacuaaucauc

SEQ ID NO: 126 - RNA, 100 nt, Artificial Sequence
  Description of Artificial Sequence Synthetic polynucleotide
gauauugugacauaucucugcacucaucaccuugccgaugcaaaucuucuauaggauccacagggagggggcauuuuaauauaucucugaacuaaucauc

SEQ ID NO: 127 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaucucugaaaaacgaauc

SEQ ID NO: 128 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaucucugaagaagagauc

SEQ ID NO: 129 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauaucucugaagcacgaauc

SEQ ID NO: 130 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaucucugaaagauaaauc

SEQ ID NO: 131 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauaucucugaaacgccaauc

SEQ ID NO: 132 - RNA, 100 nt, Artificial Sequence
  Description of Artificial Sequence Synthetic polynucleotide
gauauugugacauaucucugcacucaucacacaaaagaugcaaaucuucuauagcaggaucagggagggggcauuuuaacauaucucugaaaaaccgauc

SEQ ID NO: 133 - RNA, 100 nt, Artificial Sequence
  Description of Artificial Sequence Synthetic polynucleotide
gauauugugacauaucucugcacucaucacacaaaagaugcaaaucuucuauagccuaaucagggagggggcauuucaauauaucucugaaagcgggauc

SEQ ID NO: 134 - RNA, 100 nt, Artificial Sequence
  Description of Artificial Sequence Synthetic polynucleotide
gauauugugacauaucucugcacucaucacacaaaagaugcaaaucuucuauagcacagucagggagggggcauucuaauauaucucugaagccuaaauc

SEQ ID NO: 135 - RNA, 100 nt, Artificial Sequence
  Description of Artificial Sequence Synthetic polynucleotide
gauauugugacauaucucugcacucaucacacaaaagaugcaaaucuucuauaguuaaaucagggagggggcaucuuaauauaucucugaagccauaauc

SEQ ID NO: 136 - RNA, 100 nt, Artificial Sequence
  Description of Artificial Sequence Synthetic polynucleotide
gauauugugacauaucucugcacucaucacacaaaagaugcaaaucuucuauagccgugucagggagggggcauuuuaauauaucucugaaggccaaauc

SEQ ID NO: 137 - RNA, 100 nt, Artificial Sequence
  Description of Artificial Sequence Synthetic polynucleotide
gauauugugacauaucucugcacucaucacacaaaagaugcaaaucuucuauauaccgaacagggagggggcauuuuaacauaucucugaaagauagauc

SEQ ID NO: 138 - RNA, 100 nt, Artificial Sequence
  Description of Artificial Sequence Synthetic polynucleotide
gauauugugacauaucucugcacucaucacacaaaagaugcaaaucuucuauacacgcaacagggagggggcauucuaauauaucucugaaauuuagauc

SEQ ID NO: 139 - RNA, 100 nt, Artificial Sequence
  Description of Artificial Sequence Synthetic polynucleotide
gauauugugacauaucucugcacucaucacacaaaagaugcaaaucuucuauaaaagcaacagggagggggcaucuuaauauaucucugaaggggcaauc

SEQ ID NO: 140 - RNA, 100 nt, Artificial Sequence
  Description of Artificial Sequence Synthetic polynucleotide
gauauugugacauaucucugcacucaucacacaaaagaugcaaaucuucuauaaacgagacagggagggggcauuuuaauauaucucugaaggguaaauc

SEQ ID NO: 141 - RNA, 100 nt, Artificial Sequence
  Description of Artificial Sequence Synthetic polynucleotide
gauauugugacauaucucugcacucaucacacaaaagaugcaaaucuucuauccucaacacagggagggggcauuuuaacauaucucugaaggaaaaauc

SEQ ID NO: 142 - RNA, 100 nt, Artificial Sequence
  Description of Artificial Sequence Synthetic polynucleotide
gauauugugacauaucucugcacucaucacacaaaagaugcaaaucuucuaucgcuggcacagggagggggcauuucaauauaucucugaaggugggauc

SEQ ID NO: 143 - RNA, 100 nt, Artificial Sequence
  Description of Artificial Sequence Synthetic polynucleotide
gauauugugacauaucucugcacucaucacacaaaagaugcaaaucuucuauccagcacacagggagggggcauucuaauauaucucugaaggcacaauc

SEQ ID NO: 144 - RNA, 100 nt, Artificial Sequence
  Description of Artificial Sequence Synthetic polynucleotide
gauauugugacauaucucugcacucaucacacaaaagaugcaaaucuucuaucuuguacacagggagggggcaucuuaauauaucucugaaaucgaaauc

SEQ ID NO: 145 - RNA, 100 nt, Artificial Sequence
  Description of Artificial Sequence Synthetic polynucleotide
gauauugugacauaucucugcacucaucacacaaaagaugcaaaucuucuauccagaacacagggagggggcauuuuaauauaucucugaagauagaauc

SEQ ID NO: 146 - RNA, 100 nt, Artificial Sequence
  Description of Artificial Sequence Synthetic polynucleotide
gauauugugacauaucucugcacucaucacacaaaagaugcaaaucuucuagaaagcccacagggagggggcauuuuaacauaucucugaaagggggauc

SEQ ID NO: 147 - RNA, 100 nt, Artificial Sequence
  Description of Artificial Sequence Synthetic polynucleotide
gauauugugacauaucucugcacucaucacacaaaagaugcaaaucuucuacuccugccacagggagggggcauuucaauauaucucugaagcacggauc

SEQ ID NO: 148 - RNA, 100 nt, Artificial Sequence
  Description of Artificial Sequence Synthetic polynucleotide
gauauugugacauaucucugcacucaucacacaaaagaugcaaaucuucuaccuaacccacagggagggggcauucuaauauaucucugaaaccguaauc

SEQ ID NO: 149 - RNA, 100 nt, Artificial Sequence
  Description of Artificial Sequence Synthetic polynucleotide
gauauugugacauaucucugcacucaucacacaaaagaugcaaaucuucuaaucaucccacagggagggggcaucuuaauauaucucugaaaagcagauc

SEQ ID NO: 150 - RNA, 100 nt, Artificial Sequence
  Description of Artificial Sequence Synthetic polynucleotide
gauauugugacauaucucugcacucaucacacaaaagaugcaaaucuucuagcgaaaccacagggagggggcauuuuaauauaucucugaagaguggauc

SEQ ID NO: 151 - RNA, 100 nt, Artificial Sequence
  Description of Artificial Sequence Synthetic polynucleotide
gauauugugacauaucucugcacucaucacacaaaagaugcaaaucuucuucuacuuccacagggagggggcauuuuaacauaucucugaaaucuggauc

SEQ ID NO: 152 - RNA, 100 nt, Artificial Sequence
  Description of Artificial Sequence Synthetic polynucleotide
gauauugugacauaucucugcacucaucacacaaaagaugcaaaucuucuugccuuuccacagggagggggcauuucaauauaucucugaagguacaauc

SEQ ID NO: 153 - RNA, 100 nt, Artificial Sequence
  Description of Artificial Sequence Synthetic polynucleotide
gauauugugacauaucucugcacucaucacacaaaagaugcaaaucuucuuaccauuccacagggagggggcauucuaauauaucucugaaggagcgauc

SEQ ID NO: 154 - RNA, 100 nt, Artificial Sequence
  Description of Artificial Sequence Synthetic polynucleotide
gauauugugacauaucucugcacucaucacacaaaagaugcaaaucuucuucaccuuccacagggagggggcaucuuaauauaucucugaaggccggauc

SEQ ID NO: 155 - RNA, 100 nt, Artificial Sequence
  Description of Artificial Sequence Synthetic polynucleotide
gauauugugacauaucucugcacucaucacacaaaagaugcaaaucuucuucacccuccacagggagggggcauuuuaauauaucucugaaggcagaauc

SEQ ID NO: 156 - RNA, 100 nt, Artificial Sequence
  Description of Artificial Sequence Synthetic polynucleotide
gauauugugacauaucucugcacucaucacacaaaagaugcaaaucuucgaaaccauccacagggagggggcauuuuaacauaucucugaagacagaauc

SEQ ID NO: 157 - RNA, 100 nt, Artificial Sequence
  Description of Artificial Sequence Synthetic polynucleotide
gauauugugacauaucucugcacucaucacacaaaagaugcaaaucuucacaaauauccacagggagggggcauuucaauauaucucugaaagcgagauc

SEQ ID NO: 158 - RNA, 100 nt, Artificial Sequence
  Description of Artificial Sequence Synthetic polynucleotide
gauauugugacauaucucugcacucaucacacaaaagaugcaaaucuucccccuuauccacagggagggggcauucuaauauaucucugaaggguggauc

SEQ ID NO: 159 - RNA, 100 nt, Artificial Sequence
  Description of Artificial Sequence Synthetic polynucleotide
gauauugugacauaucucugcacucaucacacaaaagaugcaaaucuucaccccuauccacagggagggggcaucuuaauauaucucugaagacgcgauc

SEQ ID NO: 160 - RNA, 100 nt, Artificial Sequence
  Description of Artificial Sequence Synthetic polynucleotide
gauauugugacauaucucugcacucaucacacaaaagaugcaaaucuucgcaccaauccacagggagggggcauuuuaauauaucucugaagagccgauc

SEQ ID NO: 161 - RNA, 100 nt, Artificial Sequence
  Description of Artificial Sequence Synthetic polynucleotide
gauauugugacauaucucugcacucaucacacaaaagaugcaaaucuuaucgacgauccacagggagggggcauuuuaacauaucucugaaacaugaauc

SEQ ID NO: 162 - RNA, 100 nt, Artificial Sequence
  Description of Artificial Sequence Synthetic polynucleotide
gauauugugacauaucucugcacucaucacacaaaagaugcaaaucuugcccuugauccacagggagggggcauuucaauauaucucugaaacucgaauc

SEQ ID NO: 163 - RNA, 100 nt, Artificial Sequence
  Description of Artificial Sequence Synthetic polynucleotide
gauauugugacauaucucugcacucaucacacaaaagaugcaaaucuugcucgugauccacagggagggggcauucuaauauaucucugaaagcaagauc

SEQ ID NO: 164 - RNA, 100 nt, Artificial Sequence
  Description of Artificial Sequence Synthetic polynucleotide
gauauugugacauaucucugcacucaucacacaaaagaugcaaaucuuggugaagauccacagggagggggcaucuuaauauaucucugaagccagaauc

SEQ ID NO: 165 - RNA, 100 nt, Artificial Sequence
  Description of Artificial Sequence Synthetic polynucleotide
gauauugugacauaucucugcacucaucacacaaaagaugcaaaucuugcggucgauccacagggagggggcauuuuaauauaucucugaagaccggauc

SEQ ID NO: 166 - RNA, 100 nt, Artificial Sequence
  Description of Artificial Sequence Synthetic polynucleotide
gauauugugacauaucucugcacucaucacacaaaagaugcaaaucugaccucggauccacagggagggggcauuucaauauaucucugaaaucuggauc

SEQ ID NO: 167 - RNA, 100 nt, Artificial Sequence
  Description of Artificial Sequence Synthetic polynucleotide
gauauugugacauaucucugcacucaucacacaaaagaugcaaaucugauucuggauccacagggagggggcauucuaauauaucucugaagguuaaauc

SEQ ID NO: 168 - RNA, 100 nt, Artificial Sequence
  Description of Artificial Sequence Synthetic polynucleotide
gauauugugacauaucucugcacucaucacacaaaagaugcaaaucugccuuuggauccacagggagggggcaucuuaauauaucucugaaaggccgauc

SEQ ID NO: 169 - RNA, 100 nt, Artificial Sequence
  Description of Artificial Sequence Synthetic polynucleotide
gauauugugacauaucucugcacucaucacacaaaagaugcaaaucuggccucggauccacagggagggggcauuuuaauauaucucugaagcgugaauc

SEQ ID NO: 170 - RNA, 100 nt, Artificial Sequence
  Description of Artificial Sequence Synthetic polynucleotide
gauauugugacauaucucugcacucaucacacaaaagaugcaaaucguaguaaggauccacagggagggggcauuuuaacauaucucugaagaauagauc

SEQ ID NO: 171 - RNA, 100 nt, Artificial Sequence
  Description of Artificial Sequence Synthetic polynucleotide
gauauugugacauaucucugcacucaucacacaaaagaugcaaaucgacacgaggauccacagggagggggcauuucaauauaucucugaagcauggauc

SEQ ID NO: 172 - RNA, 100 nt, Artificial Sequence
  Description of Artificial Sequence Synthetic polynucleotide
gauauugugacauaucucugcacucaucacacaaaagaugcaaaucgggcgcaggauccacagggagggggcauucuaauauaucucugaagaacgaauc

SEQ ID NO: 173 - RNA, 100 nt, Artificial Sequence
  Description of Artificial Sequence Synthetic polynucleotide
gauauugugacauaucucugcacucaucacacaaaagaugcaaaucaccacgaggauccacagggagggggcaucuuaauauaucucugaaggcaaaauc

SEQ ID NO: 174 - RNA, 100 nt, Artificial Sequence
  Description of Artificial Sequence Synthetic polynucleotide
gauauugugacauaucucugcacucaucacacaaaagaugcaaaucgugagcaggauccacagggagggggcauuuuaauauaucucugaaaacgggauc

SEQ ID NO: 175 - RNA, 100 nt, Artificial Sequence
  Description of Artificial Sequence Synthetic polynucleotide
gauauugugacauaucucugcacucaucacacaaaagaugcaaaugcagguuaggauccacagggagggggcauuuuaacauaucucugaagccucgauc

SEQ ID NO: 176 - RNA, 100 nt, Artificial Sequence
  Description of Artificial Sequence Synthetic polynucleotide
gauauugugacauaucucugcacucaucacacaaaagaugcaaauagcgucuaggauccacagggagggggcauuucaauauaucucugaagggacaauc

SEQ ID NO: 177 - RNA, 100 nt, Artificial Sequence
  Description of Artificial Sequence Synthetic polynucleotide
gauauugugacauaucucugcacucaucacacaaaagaugcaaauagcaccuaggauccacagggagggggcauucuaauauaucucugaaacuccaauc

SEQ ID NO: 178 - RNA, 100 nt, Artificial Sequence
  Description of Artificial Sequence Synthetic polynucleotide
gauauugugacauaucucugcacucaucacacaaaagaugcaaauaagauuuaggauccacagggagggggcaucuuaauauaucucugaaggagcaauc

SEQ ID NO: 179 - RNA, 100 nt, Artificial Sequence
  Description of Artificial Sequence Synthetic polynucleotide
gauauugugacauaucucugcacucaucacacaaaagaugcaaaugacagcuaggauccacagggagggggcauuuuaauauaucucugaagacccgauc

SEQ ID NO: 180 - RNA, 100 nt, Artificial Sequence
  Description of Artificial Sequence Synthetic polynucleotide
gauauugugacauaucucugcacucaucacacaaaagaugcaaaagaggcauaggauccacagggagggggcauuuuaacauaucucugaaaacccgauc

SEQ ID NO: 181 - RNA, 100 nt, Artificial Sequence
  Description of Artificial Sequence Synthetic polynucleotide
gauauugugacauaucucugcacucaucacacaaaagaugcaaaguauggauaggauccacagggagggggcauuucaauauaucucugaagccuggauc

SEQ ID NO: 182 - RNA, 100 nt, Artificial Sequence
  Description of Artificial Sequence Synthetic polynucleotide
gauauugugacauaucucugcacucaucacacaaaagaugcaaaagagcgauaggauccacagggagggggcauucuaauauaucucugaaauucggauc

SEQ ID NO: 183 - RNA, 100 nt, Artificial Sequence
  Description of Artificial Sequence Synthetic polynucleotide
gauauugugacauaucucugcacucaucacacaaaagaugcaaaaguaagauaggauccacagggagggggcaucuuaauauaucucugaaauucggauc

SEQ ID NO: 184 - RNA, 100 nt, Artificial Sequence
  Description of Artificial Sequence Synthetic polynucleotide
gauauugugacauaucucugcacucaucacacaaaagaugcaaaaaccaaauaggauccacagggagggggcauuuuaauauaucucugaaagggcgauc

SEQ ID NO: 185 - RNA, 100 nt, Artificial Sequence
  Description of Artificial Sequence Synthetic polynucleotide
gauauugugacauaucucugcacucaucacacaaaagaugcaauaaauauauaggauccacagggagggggcauuuuaacauaucucugaagguuggauc

SEQ ID NO: 186 - RNA, 100 nt, Artificial Sequence
  Description of Artificial Sequence Synthetic polynucleotide
gauauugugacauaucucugcacucaucacacaaaagaugcaacaugaauauaggauccacagggagggggcaucuuaauauaucucugaaggcgagauc

SEQ ID NO: 187 - RNA, 100 nt, Artificial Sequence
  Description of Artificial Sequence Synthetic polynucleotide
gauauugugacauaucucugcacucaucacacaaaagaugcaucaagacuauaggauccacagggagggggcauuuuaacauaucucugaagccgcgauc

SEQ ID NO: 188 - RNA, 100 nt, Artificial Sequence
  Description of Artificial Sequence Synthetic polynucleotide
gauauugugacauaucucugcacucaucacacaaaagaugcacaggaccuauaggauccacagggagggggcauuucaauauaucucugaagccuaaauc

SEQ ID NO: 189 - RNA, 100 nt, Artificial Sequence
  Description of Artificial Sequence Synthetic polynucleotide
gauauugugacauaucucugcacucaucacacaaaagaugcaccuagacuauaggauccacagggagggggcauucuaauauaucucugaaaauacaauc

SEQ ID NO: 190 - RNA, 100 nt, Artificial Sequence
  Description of Artificial Sequence Synthetic polynucleotide
gauauugugacauaucucugcacucaucacacaaaagaugcacaauaacuauaggauccacagggagggggcaucuuaauauaucucugaaacccgaauc

SEQ ID NO: 191 - RNA, 100 nt, Artificial Sequence
  Description of Artificial Sequence Synthetic polynucleotide
gauauugugacauaucucugcacucaucacacaaaagaugcacugauacuauaggauccacagggagggggcauuuuaauauaucucugaagcauagauc

SEQ ID NO: 192 - RNA, 100 nt, Artificial Sequence
  Description of Artificial Sequence Synthetic polynucleotide
gauauugugacauaucucugcacucaucacacaaaagaugcuaacgcucuauaggauccacagggagggggcauuuuaacauaucucugaagccgaaauc

SEQ ID NO: 193 - RNA, 100 nt, Artificial Sequence
  Description of Artificial Sequence Synthetic polynucleotide
gauauugugacauaucucugcacucaucacacaaaagaugcuuuggcucuauaggauccacagggagggggcauuucaauauaucucugaagcgcgaauc

SEQ ID NO: 194 - RNA, 100 nt, Artificial Sequence
  Description of Artificial Sequence Synthetic polynucleotide
gauauugugacauaucucugcacucaucacacaaaagaugcccgcccucuauaggauccacagggagggggcauucuaauauaucucugaaaguaaaauc

SEQ ID NO: 195 - RNA, 100 nt, Artificial Sequence
  Description of Artificial Sequence Synthetic polynucleotide
gauauugugacauaucucugcacucaucacacaaaagaugcucgggaucuauaggauccacagggagggggcaucuuaauauaucucugaagcccggauc

SEQ ID NO: 196 - RNA, 100 nt, Artificial Sequence
  Description of Artificial Sequence Synthetic polynucleotide
gauauugugacauaucucugcacucaucacacaaaagaugccccuacucuauaggauccacagggagggggcauuuuaauauaucucugaagacaaaauc

SEQ ID NO: 197 - RNA, 100 nt, Artificial Sequence
  Description of Artificial Sequence Synthetic polynucleotide
gauauugugacauaucucugcacucaucacacaaaagaugagucgauucuauaggauccacagggagggggcauuuuaacauaucucugaagccacaauc

SEQ ID NO: 198 - RNA, 100 nt, Artificial Sequence
  Description of Artificial Sequence Synthetic polynucleotide
gauauugugacauaucucugcacucaucacacaaaagaugagggaguucuauaggauccacagggagggggcauuucaauauaucucugaaacuccaauc

SEQ ID NO: 199 - RNA, 100 nt, Artificial Sequence
  Description of Artificial Sequence Synthetic polynucleotide
gauauugugacauaucucugcacucaucacacaaaagaugauacaauucuauaggauccacagggagggggcauucuaauauaucucugaagccgcaauc

SEQ ID NO: 200 - RNA, 100 nt, Artificial Sequence
  Description of Artificial Sequence Synthetic polynucleotide
gauauugugacauaucucugcacucaucacacaaaagauggcuccguucuauaggauccacagggagggggcaucuuaauauaucucugaaacaaaaauc

SEQ ID NO: 201 - RNA, 100 nt, Artificial Sequence
  Description of Artificial Sequence Synthetic polynucleotide
gauauugugacauaucucugcacucaucacacaaaagaugaaugcguucuauaggauccacagggagggggcauuuuaauauaucucugaaagguaaauc

SEQ ID NO: 202 - RNA, 100 nt, Artificial Sequence
  Description of Artificial Sequence Synthetic polynucleotide
gauauugugacauaucucugcacucaucacacaaaagaucugucgcuucuauaggauccacagggagggggcauuuuaacauaucucugaaauguagauc

SEQ ID NO: 203 - RNA, 100 nt, Artificial Sequence
  Description of Artificial Sequence Synthetic polynucleotide
gauauugugacauaucucugcacucaucacacaaaagauuaccgacuucuauaggauccacagggagggggcauuucaauauaucucugaaaacccaauc

SEQ ID NO: 204 - RNA, 100 nt, Artificial Sequence
  Description of Artificial Sequence Synthetic polynucleotide
gauauugugacauaucucugcacucaucacacaaaagauaacuagcuucuauaggauccacagggagggggcauucuaauauaucucugaaagugcaauc

SEQ ID NO: 205 - RNA, 100 nt, Artificial Sequence
  Description of Artificial Sequence Synthetic polynucleotide
gauauugugacauaucucugcacucaucacacaaaagauaaccgacuucuauaggauccacagggagggggcaucuuaauauaucucugaagggaggauc

SEQ ID NO: 206 - RNA, 100 nt, Artificial Sequence
  Description of Artificial Sequence Synthetic polynucleotide
gauauugugacauaucucugcacucaucacacaaaagauuacgcccuucuauaggauccacagggagggggcauuuuaauauaucucugaaagcgagauc

SEQ ID NO: 207 - RNA, 100 nt, Artificial Sequence
  Description of Artificial Sequence Synthetic polynucleotide
gauauugugacauaucucugcacucaucacacaaaagaauuuccucuucuauaggauccacagggagggggcauuuuaacauaucucugaaggcgaaauc

SEQ ID NO: 208 - RNA, 100 nt, Artificial Sequence
  Description of Artificial Sequence Synthetic polynucleotide
gauauugugacauaucucugcacucaucacacaaaagaacuacuucuucuauaggauccacagggagggggcauuucaauauaucucugaaauuuaaauc

SEQ ID NO: 209 - RNA, 100 nt, Artificial Sequence
  Description of Artificial Sequence Synthetic polynucleotide
gauauugugacauaucucugcacucaucacacaaaagaggagcuucuucuauaggauccacagggagggggcauucuaauauaucucugaaaaggggauc

SEQ ID NO: 210 - RNA, 100 nt, Artificial Sequence
  Description of Artificial Sequence Synthetic polynucleotide
gauauugugacauaucucugcacucaucacacaaaagaccauauucuucuauaggauccacagggagggggcaucuuaauauaucucugaaaauaggauc

SEQ ID NO: 211 - RNA, 100 nt, Artificial Sequence
  Description of Artificial Sequence Synthetic polynucleotide
gauauugugacauaucucugcacucaucacacaaaagaauuccuucuucuauaggauccacagggagggggcauuuuaauauaucucugaagauuagauc

SEQ ID NO: 212 - RNA, 100 nt, Artificial Sequence
  Description of Artificial Sequence Synthetic polynucleotide
gauauugugacauaucucugcacucaucacacaaaagcuuaauaucuucuauaggauccacagggagggggcauuuuaacauaucucugaaaauuggauc

SEQ ID NO: 213 - RNA, 100 nt, Artificial Sequence
  Description of Artificial Sequence Synthetic polynucleotide
gauauugugacauaucucugcacucaucacacaaaagucuauuaucuucuauaggauccacagggagggggcauuucaauauaucucugaaggagggauc

SEQ ID NO: 214 - RNA, 100 nt, Artificial Sequence
  Description of Artificial Sequence Synthetic polynucleotide
gauauugugacauaucucugcacucaucacacaaaagcaauuuaucuucuauaggauccacagggagggggcaucuuaauauaucucugaagcccgaauc

SEQ ID NO: 215 - RNA, 100 nt, Artificial Sequence
  Description of Artificial Sequence Synthetic polynucleotide
gauauugugacauaucucugcacucaucacacaaaaguacucuaucuucuauaggauccacagggagggggcauuuuaauauaucucugaaggugcgauc

SEQ ID NO: 216 - RNA, 100 nt, Artificial Sequence
  Description of Artificial Sequence Synthetic polynucleotide
gauauugugacauaucucugcacucaucacacaaaacucaguaaucuucuauaggauccacagggagggggcauuuuaacauaucucugaagguggaauc

SEQ ID NO: 217 - RNA, 100 nt, Artificial Sequence
  Description of Artificial Sequence Synthetic polynucleotide
gauauugugacauaucucugcacucaucacacaaaauuuuuuaaucuucuauaggauccacagggagggggcauuucaauauaucucugaagggcagauc

SEQ ID NO: 218 - RNA, 100 nt, Artificial Sequence
  Description of Artificial Sequence Synthetic polynucleotide
gauauugugacauaucucugcacucaucacacaaaaaacaguaaucuucuauaggauccacagggagggggcauucuaauauaucucugaaacgggaauc

SEQ ID NO: 219 - RNA, 100 nt, Artificial Sequence
  Description of Artificial Sequence Synthetic polynucleotide
gauauugugacauaucucugcacucaucacacaaaaacgaacaaucuucuauaggauccacagggagggggcaucuuaauauaucucugaaggacggauc

SEQ ID NO: 220 - RNA, 100 nt, Artificial Sequence
  Description of Artificial Sequence Synthetic polynucleotide
gauauugugacauaucucugcacucaucacacaaaaaucuuuaaucuucuauaggauccacagggagggggcauuuuaauauaucucugaagaugcgauc

SEQ ID NO: 221 - RNA, 100 nt, Artificial Sequence
  Description of Artificial Sequence Synthetic polynucleotide
gauauugugacauaucucugcacucaucacacaaacucuagaaaucuucuauaggauccacagggagggggcauuuuaacauaucucugaagcggagauc

SEQ ID NO: 222 - RNA, 100 nt, Artificial Sequence
  Description of Artificial Sequence Synthetic polynucleotide
gauauugugacauaucucugcacucaucacacaaauugacaaaaucuucuauaggauccacagggagggggcauuucaauauaucucugaaaugcggauc

SEQ ID NO: 223 - RNA, 100 nt, Artificial Sequence
  Description of Artificial Sequence Synthetic polynucleotide
gauauugugacauaucucugcacucaucacacaaauucguaaaaucuucuauaggauccacagggagggggcaucuuaauauaucucugaagacaaaauc

SEQ ID NO: 224 - RNA, 100 nt, Artificial Sequence
  Description of Artificial Sequence Synthetic polynucleotide
gauauugugacauaucucugcacucaucacacaacauuaacaaaucuucuauaggauccacagggagggggcauuuuaacauaucucugaaauccgaauc

SEQ ID NO: 225 - RNA, 100 nt, Artificial Sequence
  Description of Artificial Sequence Synthetic polynucleotide
gauauugugacauaucucugcacucaucacacaauccccccaaaucuucuauaggauccacagggagggggcauuucaauauaucucugaagcucaaauc

SEQ ID NO: 226 - RNA, 100 nt, Artificial Sequence
  Description of Artificial Sequence Synthetic polynucleotide
gauauugugacauaucucugcacucaucacacaauacgcccaaaucuucuauaggauccacagggagggggcauucuaauauaucucugaaagcaaaauc

SEQ ID NO: 227 - RNA, 100 nt, Artificial Sequence
  Description of Artificial Sequence Synthetic polynucleotide
gauauugugacauaucucugcacucaucacacaacuacaucaaaucuucuauaggauccacagggagggggcaucuuaauauaucucugaaaagugaauc

SEQ ID NO: 228 - RNA, 100 nt, Artificial Sequence
  Description of Artificial Sequence Synthetic polynucleotide
gauauugugacauaucucugcacucaucacacaaccgcgccaaaucuucuauaggauccacagggagggggcauuuuaauauaucucugaagccauaauc

SEQ ID NO: 229 - RNA, 100 nt, Artificial Sequence
  Description of Artificial Sequence Synthetic polynucleotide
gauauugugacauaucucugcacucaucacacacccgcggcaaaucuucuauaggauccacagggagggggcauuuuaacauaucucugaagcacggauc

SEQ ID NO: 230 - RNA, 100 nt, Artificial Sequence
  Description of Artificial Sequence Synthetic polynucleotide
gauauugugacauaucucugcacucaucacacacgcuuagcaaaucuucuauaggauccacagggagggggcauuucaauauaucucugaaacguagauc

SEQ ID NO: 231 - RNA, 100 nt, Artificial Sequence
  Description of Artificial Sequence Synthetic polynucleotide
gauauugugacauaucucugcacucaucacacaucccaagcaaaucuucuauaggauccacagggagggggcauucuaauauaucucugaaaguuagauc

SEQ ID NO: 232 - RNA, 100 nt, Artificial Sequence
  Description of Artificial Sequence Synthetic polynucleotide
gauauugugacauaucucugcacucaucacacauuccaagcaaaucuucuauaggauccacagggagggggcaucuuaauauaucucugaaacacggauc

SEQ ID NO: 233 - RNA, 100 nt, Artificial Sequence
  Description of Artificial Sequence Synthetic polynucleotide
gauauugugacauaucucugcacucaucacacaucauuagcaaaucuucuauaggauccacagggagggggcauuuuaauauaucucugaaauugggauc

SEQ ID NO: 234 - RNA, 100 nt, Artificial Sequence
  Description of Artificial Sequence Synthetic polynucleotide
gauauugugacauaucucugcacucaucacaccguaucugcaaaucuucuauaggauccacagggagggggcauuuuaacauaucucugaagggacgauc

SEQ ID NO: 235 - RNA, 100 nt, Artificial Sequence
  Description of Artificial Sequence Synthetic polynucleotide
gauauugugacauaucucugcacucaucacaccuggccugcaaaucuucuauaggauccacagggagggggcauuucaauauaucucugaaaggcgaauc

SEQ ID NO: 236 - RNA, 100 nt, Artificial Sequence
  Description of Artificial Sequence Synthetic polynucleotide
gauauugugacauaucucugcacucaucacacuaacguugcaaaucuucuauaggauccacagggagggggcauucuaauauaucucugaaacgcagauc

SEQ ID NO: 237 - RNA, 100 nt, Artificial Sequence
  Description of Artificial Sequence Synthetic polynucleotide
gauauugugacauaucucugcacucaucacacugugcuugcaaaucuucuauaggauccacagggagggggcauuuuaauauaucucugaagccagaauc

SEQ ID NO: 238 - RNA, 100 nt, Artificial Sequence
  Description of Artificial Sequence Synthetic polynucleotide
gauauugugacauaucucugcacucaucacagccacaaugcaaaucuucuauaggauccacagggagggggcauuuuaacauaucucugaagccacaauc

SEQ ID NO: 239 - RNA, 100 nt, Artificial Sequence
  Description of Artificial Sequence Synthetic polynucleotide
gauauugugacauaucucugcacucaucacaacucucaugcaaaucuucuauaggauccacagggagggggcauucuaauauaucucugaaaauugaauc

SEQ ID NO: 240 - RNA, 100 nt, Artificial Sequence
  Description of Artificial Sequence Synthetic polynucleotide
gauauugugacauaucucugcacucaucacaauguacaugcaaaucuucuauaggauccacagggagggggcaucuuaauauaucucugaaggacagauc

SEQ ID NO: 241 - RNA, 100 nt, Artificial Sequence
  Description of Artificial Sequence Synthetic polynucleotide
gauauugugacauaucucugcacucaucacaagcgcuaugcaaaucuucuauaggauccacagggagggggcauuuuaauauaucucugaaaaacaaauc

SEQ ID NO: 242 - RNA, 100 nt, Artificial Sequence
  Description of Artificial Sequence Synthetic polynucleotide
gauauugugacauaucucugcacucaucacuauguugaugcaaaucuucuauaggauccacagggagggggcauuuuaacauaucucugaagccuagauc

SEQ ID NO: 243 - RNA, 100 nt, Artificial Sequence
  Description of Artificial Sequence Synthetic polynucleotide
gauauugugacauaucucugcacucaucacuaauccgaugcaaaucuucuauaggauccacagggagggggcauuucaauauaucucugaaggacgaauc

SEQ ID NO: 244 - RNA, 100 nt, Artificial Sequence
  Description of Artificial Sequence Synthetic polynucleotide
gauauugugacauaucucugcacucaucacuuuagugaugcaaaucuucuauaggauccacagggagggggcauucuaauauaucucugaaauccaaauc

SEQ ID NO: 245 - RNA, 100 nt, Artificial Sequence
  Description of Artificial Sequence Synthetic polynucleotide
gauauugugacauaucucugcacucaucacuauaucgaugcaaaucuucuauaggauccacagggagggggcaucuuaauauaucucugaagcccgaauc

SEQ ID NO: 246 - RNA, 100 nt, Artificial Sequence
  Description of Artificial Sequence Synthetic polynucleotide
gauauugugacauaucucugcacucaucaccuguacgaugcaaaucuucuauaggauccacagggagggggcauuuuaauauaucucugaagaagagauc

SEQ ID NO: 247 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaucucugauaaccgcauc

SEQ ID NO: 248 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaucucugaccagggcauc

SEQ ID NO: 249 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauaucucugauuguagcauc

SEQ ID NO: 250 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaucucugacaauaccauc

SEQ ID NO: 251 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauaucucugauucgugcauc

SEQ ID NO: 252 - RNA, 100 nt, Artificial Sequence
  Description of Artificial Sequence Synthetic polynucleotide
gauauugugacauaucucugcacucaucacacaaaagaugcaaaucuucuauagagcggucagggagggggcauuuuaacauaucucugauaaucccauc

SEQ ID NO: 253 - RNA, 100 nt, Artificial Sequence
  Description of Artificial Sequence Synthetic polynucleotide
gauauugugacauaucucugcacucaucacacaaaagaugcaaaucuucuauagucaggucagggagggggcauuucaauauaucucugauaauugcauc

SEQ ID NO: 254 - RNA, 100 nt, Artificial Sequence
  Description of Artificial Sequence Synthetic polynucleotide
gauauugugacauaucucugcacucaucacacaaaagaugcaaaucuucuauagacgggucagggagggggcauucuaauauaucucugauccucgcauc

SEQ ID NO: 255 - RNA, 100 nt, Artificial Sequence
  Description of Artificial Sequence Synthetic polynucleotide
gauauugugacauaucucugcacucaucacacaaaagaugcaaaucuucuauagacaaaucagggagggggcaucuuaauauaucucugacggggacauc

SEQ ID NO: 256 - RNA, 100 nt, Artificial Sequence
  Description of Artificial Sequence Synthetic polynucleotide
gauauugugacauaucucugcacucaucacacaaaagaugcaaaucuucuauagacgggucagggagggggcauuuuaauauaucucugacagcaacauc

SEQ ID NO: 257 - RNA, 100 nt, Artificial Sequence
  Description of Artificial Sequence Synthetic polynucleotide
gauauugugacauaucucugcacucaucacacaaaagaugcaaaucuucuauacacuagacagggagggggcauuuuaacauaucucugacaguuacauc

SEQ ID NO: 258 - RNA, 100 nt, Artificial Sequence
  Description of Artificial Sequence Synthetic polynucleotide
gauauugugacauaucucugcacucaucacacaaaagaugcaaaucuucuauaauuacaacagggagggggcauuucaauauaucucugacagaagcauc

SEQ ID NO: 259 - RNA, 100 nt, Artificial Sequence
  Description of Artificial Sequence Synthetic polynucleotide
gauauugugacauaucucugcacucaucacacaaaagaugcaaaucuucuauaccuuagacagggagggggcauucuaauauaucucugacgguuccauc

SEQ ID NO: 260 - RNA, 100 nt, Artificial Sequence
  Description of Artificial Sequence Synthetic polynucleotide
gauauugugacauaucucugcacucaucacacaaaagaugcaaaucuucuauauaugagacagggagggggcaucuuaauauaucucugaccccaccauc

SEQ ID NO: 261 - RNA, 100 nt, Artificial Sequence
  Description of Artificial Sequence Synthetic polynucleotide
gauauugugacauaucucugcacucaucacacaaaagaugcaaaucuucuauaaccaagacagggagggggcauuuuaauauaucucugauccuugcauc

SEQ ID NO: 262 - RNA, 100 nt, Artificial Sequence
  Description of Artificial Sequence Synthetic polynucleotide
gauauugugacauaucucugcacucaucacacaaaagaugcaaaucuucuauuuucggcacagggagggggcauuuuaacauaucucugaugacaacauc

SEQ ID NO: 263 - RNA, 100 nt, Artificial Sequence
  Description of Artificial Sequence Synthetic polynucleotide
gauauugugacauaucucugcacucaucacacaaaagaugcaaaucuucuauuuagaacacagggagggggcauuucaauauaucucugacauuugcauc

SEQ ID NO: 264 - RNA, 100 nt, Artificial Sequence
  Description of Artificial Sequence Synthetic polynucleotide
gauauugugacauaucucugcacucaucacacaaaagaugcaaaucuucuaucaccggcacagggagggggcauucuaauauaucucugacaggugcauc

SEQ ID NO: 265 - RNA, 100 nt, Artificial Sequence
  Description of Artificial Sequence Synthetic polynucleotide
gauauugugacauaucucugcacucaucacacaaaagaugcaaaucuucuauuuacagcacagggagggggcaucuuaauauaucucugacaucugcauc

SEQ ID NO: 266 - RNA, 100 nt, Artificial Sequence
  Description of Artificial Sequence Synthetic polynucleotide
gauauugugacauaucucugcacucaucacacaaaagaugcaaaucuucuauuuaugacacagggagggggcauuuuaauauaucucugauccuuccauc

SEQ ID NO: 267 - RNA, 100 nt, Artificial Sequence
  Description of Artificial Sequence Synthetic polynucleotide
gauauugugacauaucucugcacucaucacacaaaagaugcaaaucuucuacucgcaccacagggagggggcauuuuaacauaucucugauucucacauc

SEQ ID NO: 268 - RNA, 100 nt, Artificial Sequence
  Description of Artificial Sequence Synthetic polynucleotide
gauauugugacauaucucugcacucaucacacaaaagaugcaaaucuucuacgaaacccacagggagggggcauuucaauauaucucugacggugccauc

SEQ ID NO: 269 - RNA, 100 nt, Artificial Sequence
  Description of Artificial Sequence Synthetic polynucleotide
gauauugugacauaucucugcacucaucacacaaaagaugcaaaucuucuacgcacgccacagggagggggcauucuaauauaucucugauggugccauc

SEQ ID NO: 270 - RNA, 100 nt, Artificial Sequence
  Description of Artificial Sequence Synthetic polynucleotide
gauauugugacauaucucugcacucaucacacaaaagaugcaaaucuucuagucaccccacagggagggggcaucuuaauauaucucugauaccaccauc

SEQ ID NO: 271 - RNA, 100 nt, Artificial Sequence
  Description of Artificial Sequence Synthetic polynucleotide
gauauugugacauaucucugcacucaucacacaaaagaugcaaaucuucuaaugucgccacagggagggggcauuuuaauauaucucugacaacggcauc

SEQ ID NO: 272 - RNA, 100 nt, Artificial Sequence
  Description of Artificial Sequence Synthetic polynucleotide
gauauugugacauaucucugcacucaucacacaaaagaugcaaaucuucucaaugcuccacagggagggggcauuuuaacauaucucugaugaaggcauc

SEQ ID NO: 273 - RNA, 100 nt, Artificial Sequence
  Description of Artificial Sequence Synthetic polynucleotide
gauauugugacauaucucugcacucaucacacaaaagaugcaaaucuucucaucauuccacagggagggggcauuucaauauaucucugacaguaccauc

SEQ ID NO: 274 - RNA, 100 nt, Artificial Sequence
  Description of Artificial Sequence Synthetic polynucleotide
gauauugugacauaucucugcacucaucacacaaaagaugcaaaucuucucgaaguuccacagggagggggcauucuaauauaucucugauguucgcauc

SEQ ID NO: 275 - RNA, 100 nt, Artificial Sequence
  Description of Artificial Sequence Synthetic polynucleotide
gauauugugacauaucucugcacucaucacacaaaagaugcaaaucuucucgccauuccacagggagggggcaucuuaauauaucucugauagccacauc

SEQ ID NO: 276 - RNA, 100 nt, Artificial Sequence
  Description of Artificial Sequence Synthetic polynucleotide
gauauugugacauaucucugcacucaucacacaaaagaugcaaaucuucuugaauuuccacagggagggggcauuuuaauauaucucugacgaccgcauc

SEQ ID NO: 277 - RNA, 100 nt, Artificial Sequence
  Description of Artificial Sequence Synthetic polynucleotide
gauauugugacauaucucugcacucaucacacaaaagaugcaaaucuucgccgaaauccacagggagggggcauuuuaacauaucucugauaaaaacauc

SEQ ID NO: 278 - RNA, 100 nt, Artificial Sequence
  Description of Artificial Sequence Synthetic polynucleotide
gauauugugacauaucucugcacucaucacacaaaagaugcaaaucuucguguccauccacagggagggggcauuucaauauaucucugaccaugccauc

SEQ ID NO: 279 - RNA, 100 nt, Artificial Sequence
  Description of Artificial Sequence Synthetic polynucleotide
gauauugugacauaucucugcacucaucacacaaaagaugcaaaucuuccccgccauccacagggagggggcaucuuaauauaucucugaucaacccauc

SEQ ID NO: 280 - RNA, 100 nt, Artificial Sequence
  Description of Artificial Sequence Synthetic polynucleotide
gauauugugacauaucucugcacucaucacacaaaagaugcaaaucuucguucccauccacagggagggggcauuuuaauauaucucugauagucccauc

SEQ ID NO: 281 - RNA, 100 nt, Artificial Sequence
  Description of Artificial Sequence Synthetic polynucleotide
gauauugugacauaucucugcacucaucacacaaaagaugcaaaucuuagaacagauccacagggagggggcauuuuaacauaucucugaucccgacauc

SEQ ID NO: 282 - RNA, 100 nt, Artificial Sequence
  Description of Artificial Sequence Synthetic polynucleotide
gauauugugacauaucucugcacucaucacacaaaagaugcaaaucuuagcgucgauccacagggagggggcauuucaauauaucucugauguccccauc

SEQ ID NO: 283 - RNA, 100 nt, Artificial Sequence
  Description of Artificial Sequence Synthetic polynucleotide
gauauugugacauaucucugcacucaucacacaaaagaugcaaaucuugcaacagauccacagggagggggcauucuaauauaucucugauuucaacauc

SEQ ID NO: 284 - RNA, 100 nt, Artificial Sequence
  Description of Artificial Sequence Synthetic polynucleotide
gauauugugacauaucucugcacucaucacacaaaagaugcaaaucuuggacacgauccacagggagggggcaucuuaauauaucucugauggccgcauc

SEQ ID NO: 285 - RNA, 100 nt, Artificial Sequence
  Description of Artificial Sequence Synthetic polynucleotide
gauauugugacauaucucugcacucaucacacaaaagaugcaaaucuuaucauagauccacagggagggggcauuuuaauauaucucugaccagcgcauc

SEQ ID NO: 286 - RNA, 100 nt, Artificial Sequence
  Description of Artificial Sequence Synthetic polynucleotide
gauauugugacauaucucugcacucaucacacaaaagaugcaaaucugggaauggauccacagggagggggcauuuuaacauaucucugauaccgccauc

SEQ ID NO: 287 - RNA, 100 nt, Artificial Sequence
  Description of Artificial Sequence Synthetic polynucleotide
gauauugugacauaucucugcacucaucacacaaaagaugcaaaucuauggauggauccacagggagggggcauuucaauauaucucugauaguugcauc

SEQ ID NO: 288 - RNA, 100 nt, Artificial Sequence
  Description of Artificial Sequence Synthetic polynucleotide
gauauugugacauaucucugcacucaucacacaaaagaugcaaaucucacgauggauccacagggagggggcauucuaauauaucucugacaucggcauc

SEQ ID NO: 289 - RNA, 100 nt, Artificial Sequence
  Description of Artificial Sequence Synthetic polynucleotide
gauauugugacauaucucugcacucaucacacaaaagaugcaaaucuggguuuggauccacagggagggggcaucuuaauauaucucugauucccgcauc

SEQ ID NO: 290 - RNA, 100 nt, Artificial Sequence
  Description of Artificial Sequence Synthetic polynucleotide
gauauugugacauaucucugcacucaucacacaaaagaugcaaaucucaauccggauccacagggagggggcauuuuaauauaucucugacgauuacauc

SEQ ID NO: 291 - RNA, 100 nt, Artificial Sequence
  Description of Artificial Sequence Synthetic polynucleotide
gauauugugacauaucucugcacucaucacacaaaagaugcaaaucccuccaaggauccacagggagggggcauuuuaacauaucucugaugagcacauc

SEQ ID NO: 292 - RNA, 100 nt, Artificial Sequence
  Description of Artificial Sequence Synthetic polynucleotide
gauauugugacauaucucugcacucaucacacaaaagaugcaaauccuggugaggauccacagggagggggcauuucaauauaucucugauucgagcauc

SEQ ID NO: 293 - RNA, 100 nt, Artificial Sequence
  Description of Artificial Sequence Synthetic polynucleotide
gauauugugacauaucucugcacucaucacacaaaagaugcaaauccaacgcaggauccacagggagggggcauucuaauauaucucugaccaagacauc

SEQ ID NO: 294 - RNA, 100 nt, Artificial Sequence
  Description of Artificial Sequence Synthetic polynucleotide
gauauugugacauaucucugcacucaucacacaaaagaugcaaaucaaaaaaaggauccacagggagggggcaucuuaauauaucucugauuaccccauc

SEQ ID NO: 295 - RNA, 100 nt, Artificial Sequence
  Description of Artificial Sequence Synthetic polynucleotide
gauauugugacauaucucugcacucaucacacaaaagaugcaaaucaguagcaggauccacagggagggggcauuuuaauauaucucugauccucacauc

SEQ ID NO: 296 - RNA, 100 nt, Artificial Sequence
  Description of Artificial Sequence Synthetic polynucleotide
gauauugugacauaucucugcacucaucacacaaaagaugcaaauaaguacuaggauccacagggagggggcauuuuaacauaucucugauggaggcauc

SEQ ID NO: 297 - RNA, 100 nt, Artificial Sequence
  Description of Artificial Sequence Synthetic polynucleotide
gauauugugacauaucucugcacucaucacacaaaagaugcaaaugcgaauuaggauccacagggagggggcauuucaauauaucucugacagcaccauc

SEQ ID NO: 298 - RNA, 100 nt, Artificial Sequence
  Description of Artificial Sequence Synthetic polynucleotide
gauauugugacauaucucugcacucaucacacaaaagaugcaaaugaacauuaggauccacagggagggggcauucuaauauaucucugauugucgcauc

SEQ ID NO: 299 - RNA, 100 nt, Artificial Sequence
  Description of Artificial Sequence Synthetic polynucleotide
gauauugugacauaucucugcacucaucacacaaaagaugcaaauaucaauuaggauccacagggagggggcauuuuaauauaucucugauguccacauc

SEQ ID NO: 300 - RNA, 100 nt, Artificial Sequence
  Description of Artificial Sequence Synthetic polynucleotide
gauauugugacauaucucugcacucaucacacaaaagaugcaaaauauagauaggauccacagggagggggcauuuuaacauaucucugauaggcccauc

SEQ ID NO: 301 - RNA, 100 nt, Artificial Sequence
  Description of Artificial Sequence Synthetic polynucleotide
gauauugugacauaucucugcacucaucacacaaaagaugcaaaguaccaauaggauccacagggagggggcauuucaauauaucucugauguucgcauc

SEQ ID NO: 302 - RNA, 100 nt, Artificial Sequence
  Description of Artificial Sequence Synthetic polynucleotide
gauauugugacauaucucugcacucaucacacaaaagaugcaaacguaaaauaggauccacagggagggggcauucuaauauaucucugacauucccauc

SEQ ID NO: 303 - RNA, 100 nt, Artificial Sequence
  Description of Artificial Sequence Synthetic polynucleotide
gauauugugacauaucucugcacucaucacacaaaagaugcaaacaagaaauaggauccacagggagggggcaucuuaauauaucucugaugucaccauc

SEQ ID NO: 304 - RNA, 100 nt, Artificial Sequence
  Description of Artificial Sequence Synthetic polynucleotide
gauauugugacauaucucugcacucaucacacaaaagaugcaaaagggggauaggauccacagggagggggcauuuuaauauaucucugauccaggcauc

SEQ ID NO: 305 - RNA, 100 nt, Artificial Sequence
  Description of Artificial Sequence Synthetic polynucleotide
gauauugugacauaucucugcacucaucacacaaaagaugcaacagcgauauaggauccacagggagggggcauuuuaacauaucucugaucccggcauc

SEQ ID NO: 306 - RNA, 100 nt, Artificial Sequence
  Description of Artificial Sequence Synthetic polynucleotide
gauauugugacauaucucugcacucaucacacaaaagaugcaacuacaguauaggauccacagggagggggcauuucaauauaucucugacagcagcauc

SEQ ID NO: 307 - RNA, 100 nt, Artificial Sequence
  Description of Artificial Sequence Synthetic polynucleotide
gauauugugacauaucucugcacucaucacacaaaagaugcaaugagaguauaggauccacagggagggggcauucuaauauaucucugacgaaaacauc

SEQ ID NO: 308 - RNA, 100 nt, Artificial Sequence
  Description of Artificial Sequence Synthetic polynucleotide
gauauugugacauaucucugcacucaucacacaaaagaugcaaccgcaguauaggauccacagggagggggcaucuuaauauaucucugacaguuccauc

SEQ ID NO: 309 - RNA, 100 nt, Artificial Sequence
  Description of Artificial Sequence Synthetic polynucleotide
gauauugugacauaucucugcacucaucacacaaaagaugcaaugugaguauaggauccacagggagggggcauuuuaauauaucucugacagucccauc

SEQ ID NO: 310 - RNA, 100 nt, Artificial Sequence
  Description of Artificial Sequence Synthetic polynucleotide
gauauugugacauaucucugcacucaucacacaaaagaugcacgcaagcuauaggauccacagggagggggcauuuuaacauaucucugaugacagcauc

SEQ ID NO: 311 - RNA, 100 nt, Artificial Sequence
  Description of Artificial Sequence Synthetic polynucleotide
gauauugugacauaucucugcacucaucacacaaaagaugcaucagugcuauaggauccacagggagggggcauuucaauauaucucugauucggacauc

SEQ ID NO: 312 - RNA, 100 nt, Artificial Sequence
  Description of Artificial Sequence Synthetic polynucleotide
gauauugugacauaucucugcacucaucacacaaaagaugcaucuaugcuauaggauccacagggagggggcauucuaauauaucucugauuaaagcauc

SEQ ID NO: 313 - RNA, 100 nt, Artificial Sequence
  Description of Artificial Sequence Synthetic polynucleotide
gauauugugacauaucucugcacucaucacacaaaagaugcaccaggccuauaggauccacagggagggggcaucuuaauauaucucugauagcgccauc

SEQ ID NO: 314 - RNA, 100 nt, Artificial Sequence
  Description of Artificial Sequence Synthetic polynucleotide
gauauugugacauaucucugcacucaucacacaaaagaugcacuauaccuauaggauccacagggagggggcauuuuaauauaucucugauagucccauc

SEQ ID NO: 315 - RNA, 100 nt, Artificial Sequence
  Description of Artificial Sequence Synthetic polynucleotide
gauauugugacauaucucugcacucaucacacaaaagaugccuagacucuauaggauccacagggagggggcauuuuaacauaucucugacgguaccauc

SEQ ID NO: 316 - RNA, 100 nt, Artificial Sequence
  Description of Artificial Sequence Synthetic polynucleotide
gauauugugacauaucucugcacucaucacacaaaagaugcugcgacucuauaggauccacagggagggggcauuucaauauaucucugauacacgcauc

SEQ ID NO: 317 - RNA, 100 nt, Artificial Sequence
  Description of Artificial Sequence Synthetic polynucleotide
gauauugugacauaucucugcacucaucacacaaaagaugccuugacucuauaggauccacagggagggggcauucuaauauaucucugauuaugccauc

SEQ ID NO: 318 - RNA, 100 nt, Artificial Sequence
  Description of Artificial Sequence Synthetic polynucleotide
gauauugugacauaucucugcacucaucacacaaaagaugccccaagucuauaggauccacagggagggggcaucuuaauauaucucugacgaggacauc

SEQ ID NO: 319 - RNA, 100 nt, Artificial Sequence
  Description of Artificial Sequence Synthetic polynucleotide
gauauugugacauaucucugcacucaucacacaaaagaugccguauaucuauaggauccacagggagggggcauuuuaauauaucucugaugcauccauc

SEQ ID NO: 320 - RNA, 100 nt, Artificial Sequence
  Description of Artificial Sequence Synthetic polynucleotide
gauauugugacauaucucugcacucaucacacaaaagauggucgaauucuauaggauccacagggagggggcauuucaauauaucucugacagcgacauc

SEQ ID NO: 321 - RNA, 100 nt, Artificial Sequence
  Description of Artificial Sequence Synthetic polynucleotide
gauauugugacauaucucugcacucaucacacaaaagauggccccguucuauaggauccacagggagggggcauucuaauauaucucugauuauuacauc

SEQ ID NO: 322 - RNA, 100 nt, Artificial Sequence
  Description of Artificial Sequence Synthetic polynucleotide
gauauugugacauaucucugcacucaucacacaaaagaugagugaguucuauaggauccacagggagggggcaucuuaauauaucucugaugccuccauc

SEQ ID NO: 323 - RNA, 100 nt, Artificial Sequence
  Description of Artificial Sequence Synthetic polynucleotide
gauauugugacauaucucugcacucaucacacaaaagauggcccgguucuauaggauccacagggagggggcauuuuaauauaucucugacggcgacauc

SEQ ID NO: 324 - RNA, 100 nt, Artificial Sequence
  Description of Artificial Sequence Synthetic polynucleotide
gauauugugacauaucucugcacucaucacacaaaagaucucugacuucuauaggauccacagggagggggcauuuuaacauaucucugacgguuacauc

SEQ ID NO: 325 - RNA, 100 nt, Artificial Sequence
  Description of Artificial Sequence Synthetic polynucleotide
gauauugugacauaucucugcacucaucacacaaaagauauuccacuucuauaggauccacagggagggggcauuucaauauaucucugauugcgccauc

SEQ ID NO: 326 - RNA, 100 nt, Artificial Sequence
  Description of Artificial Sequence Synthetic polynucleotide
gauauugugacauaucucugcacucaucacacaaaagaucuguuccuucuauaggauccacagggagggggcauucuaauauaucucugauuacaccauc

SEQ ID NO: 327 - RNA, 100 nt, Artificial Sequence
  Description of Artificial Sequence Synthetic polynucleotide
gauauugugacauaucucugcacucaucacacaaaagauaauugccuucuauaggauccacagggagggggcauuuuaauauaucucugacaacgacauc

SEQ ID NO: 328 - RNA, 100 nt, Artificial Sequence
  Description of Artificial Sequence Synthetic polynucleotide
gauauugugacauaucucugcacucaucacacaaaagaacuuucucuucuauaggauccacagggagggggcauuucaauauaucucugauguucccauc

SEQ ID NO: 329 - RNA, 100 nt, Artificial Sequence
  Description of Artificial Sequence Synthetic polynucleotide
gauauugugacauaucucugcacucaucacacaaaagaguccucucuucuauaggauccacagggagggggcauucuaauauaucucugauagcaacauc

SEQ ID NO: 330 - RNA, 100 nt, Artificial Sequence
  Description of Artificial Sequence Synthetic polynucleotide
gauauugugacauaucucugcacucaucacacaaaagacaguguucuucuauaggauccacagggagggggcaucuuaauauaucucugauaaucccauc

SEQ ID NO: 331 - RNA, 100 nt, Artificial Sequence
  Description of Artificial Sequence Synthetic polynucleotide
gauauugugacauaucucugcacucaucacacaaaagagagguuucuucuauaggauccacagggagggggcauuuuaauauaucucugauauccacauc

SEQ ID NO: 332 - RNA, 100 nt, Artificial Sequence
  Description of Artificial Sequence Synthetic polynucleotide
gauauugugacauaucucugcacucaucacacaaaagucuaacaucuucuauaggauccacagggagggggcauuuuaacauaucucugaugcauccauc

SEQ ID NO: 333 - RNA, 100 nt, Artificial Sequence
  Description of Artificial Sequence Synthetic polynucleotide
gauauugugacauaucucugcacucaucacacaaaagccuuuuaucuucuauaggauccacagggagggggcauuucaauauaucucugacgguuacauc

SEQ ID NO: 334 - RNA, 100 nt, Artificial Sequence
  Description of Artificial Sequence Synthetic polynucleotide
gauauugugacauaucucugcacucaucacacaaaagucaaucaucuucuauaggauccacagggagggggcauucuaauauaucucugauucuuccauc

SEQ ID NO: 335 - RNA, 100 nt, Artificial Sequence
  Description of Artificial Sequence Synthetic polynucleotide
gauauugugacauaucucugcacucaucacacaaaaguuaacuaucuucuauaggauccacagggagggggcaucuuaauauaucucugacauucccauc

SEQ ID NO: 336 - RNA, 100 nt, Artificial Sequence
  Description of Artificial Sequence Synthetic polynucleotide
gauauugugacauaucucugcacucaucacacaaaagcacccuaucuucuauaggauccacagggagggggcauuuuaauauaucucugacaagggcauc

SEQ ID NO: 337 - RNA, 100 nt, Artificial Sequence
  Description of Artificial Sequence Synthetic polynucleotide
gauauugugacauaucucugcacucaucacacaaaaccaaguaaucuucuauaggauccacagggagggggcauuuuaacauaucucugauccauacauc

SEQ ID NO: 338 - RNA, 100 nt, Artificial Sequence
  Description of Artificial Sequence Synthetic polynucleotide
gauauugugacauaucucugcacucaucacacaaaacuacucaaucuucuauaggauccacagggagggggcauuucaauauaucucugacccgcccauc

SEQ ID NO: 339 - RNA, 100 nt, Artificial Sequence
  Description of Artificial Sequence Synthetic polynucleotide
gauauugugacauaucucugcacucaucacacaaaacuaccuaaucuucuauaggauccacagggagggggcauucuaauauaucucugauaaguacauc

SEQ ID NO: 340 - RNA, 100 nt, Artificial Sequence
  Description of Artificial Sequence Synthetic polynucleotide
gauauugugacauaucucugcacucaucacacaaaauuuaucaaucuucuauaggauccacagggagggggcaucuuaauauaucucugacaggcacauc

SEQ ID NO: 341 - RNA, 100 nt, Artificial Sequence
  Description of Artificial Sequence Synthetic polynucleotide
gauauugugacauaucucugcacucaucacacaaaaaucuauaaucuucuauaggauccacagggagggggcauuuuaauauaucucugauucuugcauc

SEQ ID NO: 342 - RNA, 100 nt, Artificial Sequence
  Description of Artificial Sequence Synthetic polynucleotide
gauauugugacauaucucugcacucaucacacaaacuuagaaaaucuucuauaggauccacagggagggggcauuuuaacauaucucugaugggcccauc

SEQ ID NO: 343 - RNA, 100 nt, Artificial Sequence
  Description of Artificial Sequence Synthetic polynucleotide
gauauugugacauaucucugcacucaucacacaaaucaaaaaaaucuucuauaggauccacagggagggggcauuucaauauaucucugauucuugcauc

SEQ ID NO: 344 - RNA, 100 nt, Artificial Sequence
  Description of Artificial Sequence Synthetic polynucleotide
gauauugugacauaucucugcacucaucacacaaaucgccaaaaucuucuauaggauccacagggagggggcauucuaauauaucucugauuacgacauc

SEQ ID NO: 345 - RNA, 100 nt, Artificial Sequence
  Description of Artificial Sequence Synthetic polynucleotide
gauauugugacauaucucugcacucaucacacaaacuaacgaaaucuucuauaggauccacagggagggggcaucuuaauauaucucugaucaugccauc

SEQ ID NO: 346 - RNA, 100 nt, Artificial Sequence
  Description of Artificial Sequence Synthetic polynucleotide
gauauugugacauaucucugcacucaucacacaaaucaguaaaaucuucuauaggauccacagggagggggcauuuuaauauaucucugacaaccccauc

SEQ ID NO: 347 - RNA, 100 nt, Artificial Sequence
  Description of Artificial Sequence Synthetic polynucleotide
gauauugugacauaucucugcacucaucacacaacauuuucaaaucuucuauaggauccacagggagggggcauuuuaacauaucucugauaccugcauc

SEQ ID NO: 348 - RNA, 100 nt, Artificial Sequence
  Description of Artificial Sequence Synthetic polynucleotide
gauauugugacauaucucugcacucaucacacaacaccuucaaaucuucuauaggauccacagggagggggcauuucaauauaucucugacagucgcauc

SEQ ID NO: 349 - RNA, 100 nt, Artificial Sequence
  Description of Artificial Sequence Synthetic polynucleotide
gauauugugacauaucucugcacucaucacacaaccaaaacaaaucuucuauaggauccacagggagggggcauucuaauauaucucugauucuuccauc

SEQ ID NO: 350 - RNA, 100 nt, Artificial Sequence
  Description of Artificial Sequence Synthetic polynucleotide
gauauugugacauaucucugcacucaucacacaauucaaccaaaucuucuauaggauccacagggagggggcaucuuaauauaucucugacggacacauc

SEQ ID NO: 351 - RNA, 100 nt, Artificial Sequence
  Description of Artificial Sequence Synthetic polynucleotide
gauauugugacauaucucugcacucaucacacaauaaucccaaaucuucuauaggauccacagggagggggcauuuuaauauaucucugauucggacauc

SEQ ID NO: 352 - RNA, 100 nt, Artificial Sequence
  Description of Artificial Sequence Synthetic polynucleotide
gauauugugacauaucucugcacucaucacacauuccccgcaaaucuucuauaggauccacagggagggggcauuuuaacauaucucugauaaguccauc

SEQ ID NO: 353 - RNA, 100 nt, Artificial Sequence
  Description of Artificial Sequence Synthetic polynucleotide
gauauugugacauaucucugcacucaucacacaucgugcgcaaaucuucuauaggauccacagggagggggcauuucaauauaucucugacagaggcauc

SEQ ID NO: 354 - RNA, 100 nt, Artificial Sequence
  Description of Artificial Sequence Synthetic polynucleotide
gauauugugacauaucucugcacucaucacacauaguaagcaaaucuucuauaggauccacagggagggggcaucuuaauauaucucugauucaaacauc

SEQ ID NO: 355 - RNA, 100 nt, Artificial Sequence
  Description of Artificial Sequence Synthetic polynucleotide
gauauugugacauaucucugcacucaucacacauugucggcaaaucuucuauaggauccacagggagggggcauuuuaauauaucucugaucacggcauc

SEQ ID NO: 356 - RNA, 100 nt, Artificial Sequence
  Description of Artificial Sequence Synthetic polynucleotide
gauauugugacauaucucugcacucaucacacuccuuuugcaaaucuucuauaggauccacagggagggggcauuuuaacauaucucugacgaccgcauc

SEQ ID NO: 357 - RNA, 100 nt, Artificial Sequence
  Description of Artificial Sequence Synthetic polynucleotide
gauauugugacauaucucugcacucaucacacccacuuugcaaaucuucuauaggauccacagggagggggcauuucaauauaucucugauucauacauc

SEQ ID NO: 358 - RNA, 100 nt, Artificial Sequence
  Description of Artificial Sequence Synthetic polynucleotide
gauauugugacauaucucugcacucaucacacucccucugcaaaucuucuauaggauccacagggagggggcauucuaauauaucucugacccucgcauc

SEQ ID NO: 359 - RNA, 100 nt, Artificial Sequence
  Description of Artificial Sequence Synthetic polynucleotide
gauauugugacauaucucugcacucaucacacuucaccugcaaaucuucuauaggauccacagggagggggcaucuuaauauaucucugauagcaccauc

SEQ ID NO: 360 - RNA, 100 nt, Artificial Sequence
  Description of Artificial Sequence Synthetic polynucleotide
gauauugugacauaucucugcacucaucacacuaucauugcaaaucuucuauaggauccacagggagggggcauuuuaauauaucucugaugcauacauc

SEQ ID NO: 361 - RNA, 100 nt, Artificial Sequence
  Description of Artificial Sequence Synthetic polynucleotide
gauauugugacauaucucugcacucaucacaguccauaugcaaaucuucuauaggauccacagggagggggcauuuuaacauaucucugaugcauacauc

SEQ ID NO: 362 - RNA, 100 nt, Artificial Sequence
  Description of Artificial Sequence Synthetic polynucleotide
gauauugugacauaucucugcacucaucacaaaucccaugcaaaucuucuauaggauccacagggagggggcauucuaauauaucucugaccaugccauc

SEQ ID NO: 363 - RNA, 100 nt, Artificial Sequence
  Description of Artificial Sequence Synthetic polynucleotide
gauauugugacauaucucugcacucaucacaagcauuaugcaaaucuucuauaggauccacagggagggggcaucuuaauauaucucugaugcagccauc

SEQ ID NO: 364 - RNA, 100 nt, Artificial Sequence
  Description of Artificial Sequence Synthetic polynucleotide
gauauugugacauaucucugcacucaucacaaccccaaugcaaaucuucuauaggauccacagggagggggcauuuuaauauaucucugacaucggcauc

SEQ ID NO: 365 - RNA, 100 nt, Artificial Sequence
  Description of Artificial Sequence Synthetic polynucleotide
gauauugugacauaucucugcacucaucacuaaccugaugcaaaucuucuauaggauccacagggagggggcauuuuaacauaucucugaccaaagcauc

SEQ ID NO: 366 - RNA, 100 nt, Artificial Sequence
  Description of Artificial Sequence Synthetic polynucleotide
gauauugugacauaucucugcacucaucaccuggucgaugcaaaucuucuauaggauccacagggagggggcauuucaauauaucucugauaagcccauc

SEQ ID NO: 367 - RNA, 100 nt, Artificial Sequence
  Description of Artificial Sequence Synthetic polynucleotide
gauauugugacauaucucugcacucaucaccucuuugaugcaaaucuucuauaggauccacagggagggggcauucuaauauaucucugaccaacccauc

SEQ ID NO: 368 - RNA, 100 nt, Artificial Sequence
  Description of Artificial Sequence Synthetic polynucleotide
gauauugugacauaucucugcacucaucaccucuacgaugcaaaucuucuauaggauccacagggagggggcaucuuaauauaucucugacggccccauc

SEQ ID NO: 369 - RNA, 100 nt, Artificial Sequence
  Description of Artificial Sequence Synthetic polynucleotide
gauauugugacauaucucugcacucaucacuugugugaugcaaaucuucuauaggauccacagggagggggcauuuuaauauaucucugacauucccauc

SEQ ID NO: 370 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaucucugcccagcucauc

SEQ ID NO: 371 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaucucuguuucucucauc

SEQ ID NO: 372 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauaucucugcccaucucauc

SEQ ID NO: 373 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaucucugccucgcucauc

SEQ ID NO: 374 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauaucucugcguaccucauc

SEQ ID NO: 375 - RNA, 100 nt, Artificial Sequence
  Description of Artificial Sequence Synthetic polynucleotide
gauauugugacauaucucugcacucaucacacaaaagaugcaaaucuucuauagugcggucagggagggggcauuuuaacauaucucuguugauuucauc

SEQ ID NO: 376 - RNA, 100 nt, Artificial Sequence
  Description of Artificial Sequence Synthetic polynucleotide
gauauugugacauaucucugcacucaucacacaaaagaugcaaaucuucuauaguacaaucagggagggggcauuucaauauaucucugcuuuccucauc

SEQ ID NO: 377 - RNA, 100 nt, Artificial Sequence
  Description of Artificial Sequence Synthetic polynucleotide
gauauugugacauaucucugcacucaucacacaaaagaugcaaaucuucuauagucauaucagggagggggcauucuaauauaucucugucggguucauc

SEQ ID NO: 378 - RNA, 100 nt, Artificial Sequence
  Description of Artificial Sequence Synthetic polynucleotide
gauauugugacauaucucugcacucaucacacaaaagaugcaaaucuucuauagacuaaucagggagggggcaucuuaauauaucucugcguaccucauc

SEQ ID NO: 379 - RNA, 100 nt, Artificial Sequence
  Description of Artificial Sequence Synthetic polynucleotide
gauauugugacauaucucugcacucaucacacaaaagaugcaaaucuucuauagaacaaucagggagggggcauuuuaauauaucucugcaagauucauc

SEQ ID NO: 380 - RNA, 100 nt, Artificial Sequence
  Description of Artificial Sequence Synthetic polynucleotide
gauauugugacauaucucugcacucaucacacaaaagaugcaaaucuucuauacaugaaacagggagggggcauuuuaacauaucucuguguuccucauc

SEQ ID NO: 381 - RNA, 100 nt, Artificial Sequence
  Description of Artificial Sequence Synthetic polynucleotide
gauauugugacauaucucugcacucaucacacaaaagaugcaaaucuucuauaucugcaacagggagggggcauuucaauauaucucuguuuguuucauc

SEQ ID NO: 382 - RNA, 100 nt, Artificial Sequence
  Description of Artificial Sequence Synthetic polynucleotide
gauauugugacauaucucugcacucaucacacaaaagaugcaaaucuucuauacccaagacagggagggggcauucuaauauaucucuguuaaccucauc

SEQ ID NO: 383 - RNA, 100 nt, Artificial Sequence
  Description of Artificial Sequence Synthetic polynucleotide
gauauugugacauaucucugcacucaucacacaaaagaugcaaaucuucuauaauugagacagggagggggcaucuuaauauaucucugucagucucauc

SEQ ID NO: 384 - RNA, 100 nt, Artificial Sequence
  Description of Artificial Sequence Synthetic polynucleotide
gauauugugacauaucucugcacucaucacacaaaagaugcaaaucuucuaucgcuagcacagggagggggcauuuuaacauaucucugugaguuucauc

SEQ ID NO: 385 - RNA, 100 nt, Artificial Sequence
  Description of Artificial Sequence Synthetic polynucleotide
gauauugugacauaucucugcacucaucacacaaaagaugcaaaucuucuauuuccaacacagggagggggcauuucaauauaucucuguugggcucauc

SEQ ID NO: 386 - RNA, 100 nt, Artificial Sequence
  Description of Artificial Sequence Synthetic polynucleotide
gauauugugacauaucucugcacucaucacacaaaagaugcaaaucuucuauuaaaagcacagggagggggcauucuaauauaucucugugagacucauc

SEQ ID NO: 387 - RNA, 100 nt, Artificial Sequence
  Description of Artificial Sequence Synthetic polynucleotide
gauauugugacauaucucugcacucaucacacaaaagaugcaaaucuucuaucccgaacacagggagggggcaucuuaauauaucucugcguauuucauc

SEQ ID NO: 388 - RNA, 100 nt, Artificial Sequence
  Description of Artificial Sequence Synthetic polynucleotide
gauauugugacauaucucugcacucaucacacaaaagaugcaaaucuucuaucaugcgcacagggagggggcauuuuaauauaucucugcgggauucauc

SEQ ID NO: 389 - RNA, 100 nt, Artificial Sequence
  Description of Artificial Sequence Synthetic polynucleotide
gauauugugacauaucucugcacucaucacacaaaagaugcaaaucuucuacccuggccacagggagggggcauuuuaacauaucucugcccaccucauc

SEQ ID NO: 390 - RNA, 100 nt, Artificial Sequence
  Description of Artificial Sequence Synthetic polynucleotide
gauauugugacauaucucugcacucaucacacaaaagaugcaaaucuucuaaaauccccacagggagggggcauuucaauauaucucugucccgcucauc

SEQ ID NO: 391 - RNA, 100 nt, Artificial Sequence
  Description of Artificial Sequence Synthetic polynucleotide
gauauugugacauaucucugcacucaucacacaaaagaugcaaaucuucuacucaugccacagggagggggcauucuaauauaucucuguuaaguucauc

SEQ ID NO: 392 - RNA, 100 nt, Artificial Sequence
  Description of Artificial Sequence Synthetic polynucleotide
gauauugugacauaucucugcacucaucacacaaaagaugcaaaucuucuaauuaugccacagggagggggcaucuuaauauaucucugugucuuucauc

SEQ ID NO: 393 - RNA, 100 nt, Artificial Sequence
  Description of Artificial Sequence Synthetic polynucleotide
gauauugugacauaucucugcacucaucacacaaaagaugcaaaucuucuaggccucccacagggagggggcauuuuaauauaucucugccaccuucauc

SEQ ID NO: 394 - RNA, 100 nt, Artificial Sequence
  Description of Artificial Sequence Synthetic polynucleotide
gauauugugacauaucucugcacucaucacacaaaagaugcaaaucuucuucauccuccacagggagggggcauuuuaacauaucucuguuacacucauc

SEQ ID NO: 395 - RNA, 100 nt, Artificial Sequence
  Description of Artificial Sequence Synthetic polynucleotide
gauauugugacauaucucugcacucaucacacaaaagaugcaaaucuucuccguuuuccacagggagggggcauuucaauauaucucuguugccuucauc

SEQ ID NO: 396 - RNA, 100 nt, Artificial Sequence
  Description of Artificial Sequence Synthetic polynucleotide
gauauugugacauaucucugcacucaucacacaaaagaugcaaaucuucuugccguuccacagggagggggcauucuaauauaucucugcggaacucauc

SEQ ID NO: 397 - RNA, 100 nt, Artificial Sequence
  Description of Artificial Sequence Synthetic polynucleotide
gauauugugacauaucucugcacucaucacacaaaagaugcaaaucuucuuccaauuccacagggagggggcaucuuaauauaucucugcgauccucauc

SEQ ID NO: 398 - RNA, 100 nt, Artificial Sequence
  Description of Artificial Sequence Synthetic polynucleotide
gauauugugacauaucucugcacucaucacacaaaagaugcaaaucuucucauuuuuccacagggagggggcauuuuaauauaucucugugucguucauc

SEQ ID NO: 399 - RNA, 100 nt, Artificial Sequence
  Description of Artificial Sequence Synthetic polynucleotide
gauauugugacauaucucugcacucaucacacaaaagaugcaaaucuucgccgcaauccacagggagggggcauuuuaacauaucucugucggacucauc

SEQ ID NO: 400 - RNA, 100 nt, Artificial Sequence
  Description of Artificial Sequence Synthetic polynucleotide
gauauugugacauaucucugcacucaucacacaaaagaugcaaaucuucagcuauauccacagggagggggcauuucaauauaucucugccaaauucauc

SEQ ID NO: 401 - RNA, 100 nt, Artificial Sequence
  Description of Artificial Sequence Synthetic polynucleotide
gauauugugacauaucucugcacucaucacacaaaagaugcaaaucuucgaaacuauccacagggagggggcauucuaauauaucucugcugguuucauc

SEQ ID NO: 402 - RNA, 100 nt, Artificial Sequence
  Description of Artificial Sequence Synthetic polynucleotide
gauauugugacauaucucugcacucaucacacaaaagaugcaaaucuuccgauucauccacagggagggggcaucuuaauauaucucugucaaucucauc

SEQ ID NO: 403 - RNA, 100 nt, Artificial Sequence
  Description of Artificial Sequence Synthetic polynucleotide
gauauugugacauaucucugcacucaucacacaaaagaugcaaaucuugccucagauccacagggagggggcauuuuaacauaucucuguuggauucauc

SEQ ID NO: 404 - RNA, 100 nt, Artificial Sequence
  Description of Artificial Sequence Synthetic polynucleotide
gauauugugacauaucucugcacucaucacacaaaagaugcaaaucuuaaagcugauccacagggagggggcauuucaauauaucucugucguccucauc

SEQ ID NO: 405 - RNA, 100 nt, Artificial Sequence
  Description of Artificial Sequence Synthetic polynucleotide
gauauugugacauaucucugcacucaucacacaaaagaugcaaaucuuaccgaagauccacagggagggggcauucuaauauaucucugccgucuucauc

SEQ ID NO: 406 - RNA, 100 nt, Artificial Sequence
  Description of Artificial Sequence Synthetic polynucleotide
gauauugugacauaucucugcacucaucacacaaaagaugcaaaucuuauccucgauccacagggagggggcaucuuaauauaucucugucaaguucauc

SEQ ID NO: 407 - RNA, 100 nt, Artificial Sequence
  Description of Artificial Sequence Synthetic polynucleotide
gauauugugacauaucucugcacucaucacacaaaagaugcaaaucuuagugcagauccacagggagggggcauuuuaauauaucucugcggaucucauc

SEQ ID NO: 408 - RNA, 100 nt, Artificial Sequence
  Description of Artificial Sequence Synthetic polynucleotide
gauauugugacauaucucugcacucaucacacaaaagaugcaaaucuaaccccggauccacagggagggggcauuuuaacauaucucugccucgcucauc

SEQ ID NO: 409 - RNA, 100 nt, Artificial Sequence
  Description of Artificial Sequence Synthetic polynucleotide
gauauugugacauaucucugcacucaucacacaaaagaugcaaaucugggagcggauccacagggagggggcauuucaauauaucucuguuacauucauc

SEQ ID NO: 410 - RNA, 100 nt, Artificial Sequence
  Description of Artificial Sequence Synthetic polynucleotide
gauauugugacauaucucugcacucaucacacaaaagaugcaaaucucacgauggauccacagggagggggcauucuaauauaucucugugagccucauc

SEQ ID NO: 411 - RNA, 100 nt, Artificial Sequence
  Description of Artificial Sequence Synthetic polynucleotide
gauauugugacauaucucugcacucaucacacaaaagaugcaaaucucauccuggauccacagggagggggcauuuuaauauaucucugcguaucucauc

SEQ ID NO: 412 - RNA, 100 nt, Artificial Sequence
  Description of Artificial Sequence Synthetic polynucleotide
gauauugugacauaucucugcacucaucacacaaaagaugcaaaucaauacaaggauccacagggagggggcauuuuaacauaucucugcggaucucauc

SEQ ID NO: 413 - RNA, 100 nt, Artificial Sequence
  Description of Artificial Sequence Synthetic polynucleotide
gauauugugacauaucucugcacucaucacacaaaagaugcaaaucggagagaggauccacagggagggggcauuucaauauaucucugccaggcucauc

SEQ ID NO: 414 - RNA, 100 nt, Artificial Sequence
  Description of Artificial Sequence Synthetic polynucleotide
gauauugugacauaucucugcacucaucacacaaaagaugcaaauccggcgcaggauccacagggagggggcauucuaauauaucucugcagauuucauc

SEQ ID NO: 415 - RNA, 100 nt, Artificial Sequence
  Description of Artificial Sequence Synthetic polynucleotide
gauauugugacauaucucugcacucaucacacaaaagaugcaaaucgcgcaaaggauccacagggagggggcaucuuaauauaucucuguaaagcucauc

SEQ ID NO: 416 - RNA, 100 nt, Artificial Sequence
  Description of Artificial Sequence Synthetic polynucleotide
gauauugugacauaucucugcacucaucacacaaaagaugcaaaucguggugaggauccacagggagggggcauuuuaauauaucucugccgagcucauc

SEQ ID NO: 417 - RNA, 100 nt, Artificial Sequence
  Description of Artificial Sequence Synthetic polynucleotide
gauauugugacauaucucugcacucaucacacaaaagaugcaaaugcauuuuaggauccacagggagggggcauuuuaacauaucucuguuaaacucauc

SEQ ID NO: 418 - RNA, 100 nt, Artificial Sequence
  Description of Artificial Sequence Synthetic polynucleotide
gauauugugacauaucucugcacucaucacacaaaagaugcaaaugggaauuaggauccacagggagggggcauuucaauauaucucugugaaccucauc

SEQ ID NO: 419 - RNA, 100 nt, Artificial Sequence
  Description of Artificial Sequence Synthetic polynucleotide
gauauugugacauaucucugcacucaucacacaaaagaugcaaaugcuaauuaggauccacagggagggggcauucuaauauaucucugucacccucauc

SEQ ID NO: 420 - RNA, 100 nt, Artificial Sequence
  Description of Artificial Sequence Synthetic polynucleotide
gauauugugacauaucucugcacucaucacacaaaagaugcaaauauggauuaggauccacagggagggggcaucuuaauauaucucugccgguuucauc

SEQ ID NO: 421 - RNA, 100 nt, Artificial Sequence
  Description of Artificial Sequence Synthetic polynucleotide
gauauugugacauaucucugcacucaucacacaaaagaugcaaauaauaccuaggauccacagggagggggcauuuuaauauaucucuguuuaucucauc

SEQ ID NO: 422 - RNA, 100 nt, Artificial Sequence
  Description of Artificial Sequence Synthetic polynucleotide
gauauugugacauaucucugcacucaucacacaaaagaugcaaacagcacauaggauccacagggagggggcauuuuaacauaucucugcuggauucauc

SEQ ID NO: 423 - RNA, 100 nt, Artificial Sequence
  Description of Artificial Sequence Synthetic polynucleotide
gauauugugacauaucucugcacucaucacacaaaagaugcaaaacaaacauaggauccacagggagggggcauuucaauauaucucugcccccuucauc

SEQ ID NO: 424 - RNA, 100 nt, Artificial Sequence
  Description of Artificial Sequence Synthetic polynucleotide
gauauugugacauaucucugcacucaucacacaaaagaugcaaaagaggcauaggauccacagggagggggcauucuaauauaucucugcuugccucauc

SEQ ID NO: 425 - RNA, 100 nt, Artificial Sequence
  Description of Artificial Sequence Synthetic polynucleotide
gauauugugacauaucucugcacucaucacacaaaagaugcaaacacggcauaggauccacagggagggggcaucuuaauauaucucugucgguuucauc

SEQ ID NO: 426 - RNA, 100 nt, Artificial Sequence
  Description of Artificial Sequence Synthetic polynucleotide
gauauugugacauaucucugcacucaucacacaaaagaugcaaaauuagaauaggauccacagggagggggcauuuuaauauaucucuguugcauucauc

SEQ ID NO: 427 - RNA, 100 nt, Artificial Sequence
  Description of Artificial Sequence Synthetic polynucleotide
gauauugugacauaucucugcacucaucacacaaaagaugcaauaagcguauaggauccacagggagggggcauuuuaacauaucucugcccaucucauc

SEQ ID NO: 428 - RNA, 100 nt, Artificial Sequence
  Description of Artificial Sequence Synthetic polynucleotide
gauauugugacauaucucugcacucaucacacaaaagaugcaacauacauauaggauccacagggagggggcauuucaauauaucucugcgugauucauc

SEQ ID NO: 429 - RNA, 100 nt, Artificial Sequence
  Description of Artificial Sequence Synthetic polynucleotide
gauauugugacauaucucugcacucaucacacaaaagaugcaauguaaguauaggauccacagggagggggcauucuaauauaucucugucaucuucauc

SEQ ID NO: 430 - RNA, 100 nt, Artificial Sequence
  Description of Artificial Sequence Synthetic polynucleotide
gauauugugacauaucucugcacucaucacacaaaagaugcaacuaaaguauaggauccacagggagggggcaucuuaauauaucucuguaaagcucauc

SEQ ID NO: 431 - RNA, 100 nt, Artificial Sequence
  Description of Artificial Sequence Synthetic polynucleotide
gauauugugacauaucucugcacucaucacacaaaagaugcaauugcuauauaggauccacagggagggggcauuuuaauauaucucuguuuaccucauc

SEQ ID NO: 432 - RNA, 100 nt, Artificial Sequence
  Description of Artificial Sequence Synthetic polynucleotide
gauauugugacauaucucugcacucaucacacaaaagaugcaccauaacuauaggauccacagggagggggcauuuuaacauaucucugucaaguucauc

SEQ ID NO: 433 - RNA, 100 nt, Artificial Sequence
  Description of Artificial Sequence Synthetic polynucleotide
gauauugugacauaucucugcacucaucacacaaaagaugcaucuaagcuauaggauccacagggagggggcauuucaauauaucucugcccccuucauc

SEQ ID NO: 434 - RNA, 100 nt, Artificial Sequence
  Description of Artificial Sequence Synthetic polynucleotide
gauauugugacauaucucugcacucaucacacaaaagaugcaucgaaccuauaggauccacagggagggggcauucuaauauaucucugccccauucauc

SEQ ID NO: 435 - RNA, 100 nt, Artificial Sequence
  Description of Artificial Sequence Synthetic polynucleotide
gauauugugacauaucucugcacucaucacacaaaagaugcacgaaaccuauaggauccacagggagggggcaucuuaauauaucucuguuggccucauc

SEQ ID NO: 436 - RNA, 100 nt, Artificial Sequence
  Description of Artificial Sequence Synthetic polynucleotide
gauauugugacauaucucugcacucaucacacaaaagaugcaucagggcuauaggauccacagggagggggcauuuuaauauaucucugcuggauucauc

SEQ ID NO: 437 - RNA, 100 nt, Artificial Sequence
  Description of Artificial Sequence Synthetic polynucleotide
gauauugugacauaucucugcacucaucacacaaaagaugccagaggucuauaggauccacagggagggggcauuuuaacauaucucuguugaguucauc

SEQ ID NO: 438 - RNA, 100 nt, Artificial Sequence
  Description of Artificial Sequence Synthetic polynucleotide
gauauugugacauaucucugcacucaucacacaaaagaugcuucggcucuauaggauccacagggagggggcauuucaauauaucucugcgggucucauc

SEQ ID NO: 439 - RNA, 100 nt, Artificial Sequence
  Description of Artificial Sequence Synthetic polynucleotide
gauauugugacauaucucugcacucaucacacaaaagaugcccacugucuauaggauccacagggagggggcauucuaauauaucucugcugguuucauc

SEQ ID NO: 440 - RNA, 100 nt, Artificial Sequence
  Description of Artificial Sequence Synthetic polynucleotide
gauauugugacauaucucugcacucaucacacaaaagaugcuuaacgucuauaggauccacagggagggggcaucuuaauauaucucugucagucucauc

SEQ ID NO: 441 - RNA, 100 nt, Artificial Sequence
  Description of Artificial Sequence Synthetic polynucleotide
gauauugugacauaucucugcacucaucacacaaaagaugcccacagucuauaggauccacagggagggggcauuuuaauauaucucugcgaauuucauc

SEQ ID NO: 442 - RNA, 100 nt, Artificial Sequence
  Description of Artificial Sequence Synthetic polynucleotide
gauauugugacauaucucugcacucaucacacaaaagaugguuguauucuauaggauccacagggagggggcauuuuaacauaucucuguuucucucauc

SEQ ID NO: 443 - RNA, 100 nt, Artificial Sequence
  Description of Artificial Sequence Synthetic polynucleotide
gauauugugacauaucucugcacucaucacacaaaagaugacugcguucuauaggauccacagggagggggcauuucaauauaucucuguuacacucauc

SEQ ID NO: 444 - RNA, 100 nt, Artificial Sequence
  Description of Artificial Sequence Synthetic polynucleotide
gauauugugacauaucucugcacucaucacacaaaagauggccagguucuauaggauccacagggagggggcauucuaauauaucucugugauccucauc

SEQ ID NO: 445 - RNA, 100 nt, Artificial Sequence
  Description of Artificial Sequence Synthetic polynucleotide
gauauugugacauaucucugcacucaucacacaaaagaugagucaauucuauaggauccacagggagggggcaucuuaauauaucucugcuuaucucauc

SEQ ID NO: 446 - RNA, 100 nt, Artificial Sequence
  Description of Artificial Sequence Synthetic polynucleotide
gauauugugacauaucucugcacucaucacacaaaagauggcgcaguucuauaggauccacagggagggggcauuuuaauauaucucuguccaucucauc

SEQ ID NO: 447 - RNA, 100 nt, Artificial Sequence
  Description of Artificial Sequence Synthetic polynucleotide
gauauugugacauaucucugcacucaucacacaaaagauuagucacuucuauaggauccacagggagggggcauuuuaacauaucucuguugguuucauc

SEQ ID NO: 448 - RNA, 100 nt, Artificial Sequence
  Description of Artificial Sequence Synthetic polynucleotide
gauauugugacauaucucugcacucaucacacaaaagauauguuccuucuauaggauccacagggagggggcauuucaauauaucucugucaaauucauc

SEQ ID NO: 449 - RNA, 100 nt, Artificial Sequence
  Description of Artificial Sequence Synthetic polynucleotide
gauauugugacauaucucugcacucaucacacaaaagauuaacggcuucuauaggauccacagggagggggcauucuaauauaucucugcggaucucauc

SEQ ID NO: 450 - RNA, 100 nt, Artificial Sequence
  Description of Artificial Sequence Synthetic polynucleotide
gauauugugacauaucucugcacucaucacacaaaagauuuucaccuucuauaggauccacagggagggggcaucuuaauauaucucuguaagguucauc

SEQ ID NO: 451 - RNA, 100 nt, Artificial Sequence
  Description of Artificial Sequence Synthetic polynucleotide
gauauugugacauaucucugcacucaucacacaaaagauaaauagcuucuauaggauccacagggagggggcauuuuaauauaucucuguuuauuucauc

SEQ ID NO: 452 - RNA, 100 nt, Artificial Sequence
  Description of Artificial Sequence Synthetic polynucleotide
gauauugugacauaucucugcacucaucacacaaaagaauuuauucuucuauaggauccacagggagggggcauuuuaacauaucucugcccaccucauc

SEQ ID NO: 453 - RNA, 100 nt, Artificial Sequence
  Description of Artificial Sequence Synthetic polynucleotide
gauauugugacauaucucugcacucaucacacaaaagaguccgcucuucuauaggauccacagggagggggcauuucaauauaucucuguuggucucauc

SEQ ID NO: 454 - RNA, 100 nt, Artificial Sequence
  Description of Artificial Sequence Synthetic polynucleotide
gauauugugacauaucucugcacucaucacacaaaagaacauguucuucuauaggauccacagggagggggcauucuaauauaucucuguauaucucauc

SEQ ID NO: 455 - RNA, 100 nt, Artificial Sequence
  Description of Artificial Sequence Synthetic polynucleotide
gauauugugacauaucucugcacucaucacacaaaagaccccuuucuucuauaggauccacagggagggggcaucuuaauauaucucugcgaaguucauc

SEQ ID NO: 456 - RNA, 100 nt, Artificial Sequence
  Description of Artificial Sequence Synthetic polynucleotide
gauauugugacauaucucugcacucaucacacaaaagaaagucuucuucuauaggauccacagggagggggcauuuuaauauaucucuguagggcucauc

SEQ ID NO: 457 - RNA, 100 nt, Artificial Sequence
  Description of Artificial Sequence Synthetic polynucleotide
gauauugugacauaucucugcacucaucacacaaaaguauaguaucuucuauaggauccacagggagggggcauuuuaacauaucucuguugcccucauc

SEQ ID NO: 458 - RNA, 100 nt, Artificial Sequence
  Description of Artificial Sequence Synthetic polynucleotide
gauauugugacauaucucugcacucaucacacaaaaguacuacaucuucuauaggauccacagggagggggcauuucaauauaucucuguguguuucauc

SEQ ID NO: 459 - RNA, 100 nt, Artificial Sequence
  Description of Artificial Sequence Synthetic polynucleotide
gauauugugacauaucucugcacucaucacacaaaagcgauucaucuucuauaggauccacagggagggggcauucuaauauaucucugcccccuucauc

SEQ ID NO: 460 - RNA, 100 nt, Artificial Sequence
  Description of Artificial Sequence Synthetic polynucleotide
gauauugugacauaucucugcacucaucacacaaaagcaaaucaucuucuauaggauccacagggagggggcaucuuaauauaucucugccaaacucauc

SEQ ID NO: 461 - RNA, 100 nt, Artificial Sequence
  Description of Artificial Sequence Synthetic polynucleotide
gauauugugacauaucucugcacucaucacacaaaaguaaaauaucuucuauaggauccacagggagggggcauuuuaauauaucucuguuauccucauc

SEQ ID NO: 462 - RNA, 100 nt, Artificial Sequence
  Description of Artificial Sequence Synthetic polynucleotide
gauauugugacauaucucugcacucaucacacaaaacuccacaaucuucuauaggauccacagggagggggcauuuuaacauaucucuguaucucucauc

SEQ ID NO: 463 - RNA, 100 nt, Artificial Sequence
  Description of Artificial Sequence Synthetic polynucleotide
gauauugugacauaucucugcacucaucacacaaaacuaacuaaucuucuauaggauccacagggagggggcauuucaauauaucucuguugcauucauc

SEQ ID NO: 464 - RNA, 100 nt, Artificial Sequence
  Description of Artificial Sequence Synthetic polynucleotide
gauauugugacauaucucugcacucaucacacaaaaccacuuaaucuucuauaggauccacagggagggggcauucuaauauaucucugccaguuucauc

SEQ ID NO: 465 - RNA, 100 nt, Artificial Sequence
  Description of Artificial Sequence Synthetic polynucleotide
gauauugugacauaucucugcacucaucacacaaaauacuauaaucuucuauaggauccacagggagggggcaucuuaauauaucucuguugucuucauc

SEQ ID NO: 466 - RNA, 100 nt, Artificial Sequence
  Description of Artificial Sequence Synthetic polynucleotide
gauauugugacauaucucugcacucaucacacaaaaaucaacaaucuucuauaggauccacagggagggggcauuuuaauauaucucugucuuccucauc

SEQ ID NO: 467 - RNA, 100 nt, Artificial Sequence
  Description of Artificial Sequence Synthetic polynucleotide
gauauugugacauaucucugcacucaucacacaaacuaguaaaaucuucuauaggauccacagggagggggcauuuuaacauaucucugccgucuucauc

SEQ ID NO: 468 - RNA, 100 nt, Artificial Sequence
  Description of Artificial Sequence Synthetic polynucleotide
gauauugugacauaucucugcacucaucacacaaacuucagaaaucuucuauaggauccacagggagggggcauuucaauauaucucuguuucucucauc

SEQ ID NO: 469 - RNA, 100 nt, Artificial Sequence
  Description of Artificial Sequence Synthetic polynucleotide
gauauugugacauaucucugcacucaucacacaaauuuggaaaaucuucuauaggauccacagggagggggcauucuaauauaucucugcuuaguucauc

SEQ ID NO: 470 - RNA, 100 nt, Artificial Sequence
  Description of Artificial Sequence Synthetic polynucleotide
gauauugugacauaucucugcacucaucacacaaacaccaaaaaucuucuauaggauccacagggagggggcaucuuaauauaucucuguuugucucauc

SEQ ID NO: 471 - RNA, 100 nt, Artificial Sequence
  Description of Artificial Sequence Synthetic polynucleotide
gauauugugacauaucucugcacucaucacacaaauucuuaaaaucuucuauaggauccacagggagggggcauuuuaauauaucucugccagucucauc

SEQ ID NO: 472 - RNA, 100 nt, Artificial Sequence
  Description of Artificial Sequence Synthetic polynucleotide
gauauugugacauaucucugcacucaucacacaaccuacacaaaucuucuauaggauccacagggagggggcauuuuaacauaucucuguugcacucauc

SEQ ID NO: 473 - RNA, 100 nt, Artificial Sequence
  Description of Artificial Sequence Synthetic polynucleotide
gauauugugacauaucucugcacucaucacacaacgaaaucaaaucuucuauaggauccacagggagggggcauuucaauauaucucugccaccuucauc

SEQ ID NO: 474 - RNA, 100 nt, Artificial Sequence
  Description of Artificial Sequence Synthetic polynucleotide
gauauugugacauaucucugcacucaucacacaauacacucaaaucuucuauaggauccacagggagggggcauucuaauauaucucugccaaucucauc

SEQ ID NO: 475 - RNA, 100 nt, Artificial Sequence
  Description of Artificial Sequence Synthetic polynucleotide
gauauugugacauaucucugcacucaucacacaauuuagccaaaucuucuauaggauccacagggagggggcaucuuaauauaucucugcaggacucauc

SEQ ID NO: 476 - RNA, 100 nt, Artificial Sequence
  Description of Artificial Sequence Synthetic polynucleotide
gauauugugacauaucucugcacucaucacacaacgaaaccaaaucuucuauaggauccacagggagggggcauuuuaauauaucucuguuuaccucauc

SEQ ID NO: 477 - RNA, 100 nt, Artificial Sequence
  Description of Artificial Sequence Synthetic polynucleotide
gauauugugacauaucucugcacucaucacacauuuccagcaaaucuucuauaggauccacagggagggggcauuuuaacauaucucugcuuauuucauc

SEQ ID NO: 478 - RNA, 100 nt, Artificial Sequence
  Description of Artificial Sequence Synthetic polynucleotide
gauauugugacauaucucugcacucaucacacacacguggcaaaucuucuauaggauccacagggagggggcauuucaauauaucucugcgggguucauc

SEQ ID NO: 479 - RNA, 100 nt, Artificial Sequence
  Description of Artificial Sequence Synthetic polynucleotide
gauauugugacauaucucugcacucaucacacaccacacgcaaaucuucuauaggauccacagggagggggcauucuaauauaucucuguuuuccucauc

SEQ ID NO: 480 - RNA, 100 nt, Artificial Sequence
  Description of Artificial Sequence Synthetic polynucleotide
gauauugugacauaucucugcacucaucacacaccuuucgcaaaucuucuauaggauccacagggagggggcaucuuaauauaucucugucaucuucauc

SEQ ID NO: 481 - RNA, 100 nt, Artificial Sequence
  Description of Artificial Sequence Synthetic polynucleotide
gauauugugacauaucucugcacucaucacacaucgccggcaaaucuucuauaggauccacagggagggggcauuuuaauauaucucugccccccucauc

SEQ ID NO: 482 - RNA, 100 nt, Artificial Sequence
  Description of Artificial Sequence Synthetic polynucleotide
gauauugugacauaucucugcacucaucacacuacgccugcaaaucuucuauaggauccacagggagggggcauuuuaacauaucucuguuacauucauc

SEQ ID NO: 483 - RNA, 100 nt, Artificial Sequence
  Description of Artificial Sequence Synthetic polynucleotide
gauauugugacauaucucugcacucaucacacccugucugcaaaucuucuauaggauccacagggagggggcauuucaauauaucucuguccacuucauc

SEQ ID NO: 484 - RNA, 100 nt, Artificial Sequence
  Description of Artificial Sequence Synthetic polynucleotide
gauauugugacauaucucugcacucaucacacuccauuugcaaaucuucuauaggauccacagggagggggcauucuaauauaucucugucucgcucauc

SEQ ID NO: 485 - RNA, 100 nt, Artificial Sequence
  Description of Artificial Sequence Synthetic polynucleotide
gauauugugacauaucucugcacucaucacacuccaucugcaaaucuucuauaggauccacagggagggggcaucuuaauauaucucuguguauuucauc

SEQ ID NO: 486 - RNA, 100 nt, Artificial Sequence
  Description of Artificial Sequence Synthetic polynucleotide
gauauugugacauaucucugcacucaucacacugucccugcaaaucuucuauaggauccacagggagggggcauuuuaauauaucucugccagucucauc

SEQ ID NO: 487 - RNA, 100 nt, Artificial Sequence
  Description of Artificial Sequence Synthetic polynucleotide
gauauugugacauaucucugcacucaucacaaucgcuaugcaaaucuucuauaggauccacagggagggggcauuuuaacauaucucugccucacucauc

SEQ ID NO: 488 - RNA, 100 nt, Artificial Sequence
  Description of Artificial Sequence Synthetic polynucleotide
gauauugugacauaucucugcacucaucacaagccaaaugcaaaucuucuauaggauccacagggagggggcauuucaauauaucucugccguccucauc

SEQ ID NO: 489 - RNA, 100 nt, Artificial Sequence
  Description of Artificial Sequence Synthetic polynucleotide
gauauugugacauaucucugcacucaucacaguucucaugcaaaucuucuauaggauccacagggagggggcauucuaauauaucucuguuucuuucauc

SEQ ID NO: 490 - RNA, 100 nt, Artificial Sequence
  Description of Artificial Sequence Synthetic polynucleotide
gauauugugacauaucucugcacucaucacagcucauaugcaaaucuucuauaggauccacagggagggggcaucuuaauauaucucugcuucucucauc

SEQ ID NO: 491 - RNA, 100 nt, Artificial Sequence
  Description of Artificial Sequence Synthetic polynucleotide
gauauugugacauaucucugcacucaucacaaaguuuaugcaaaucuucuauaggauccacagggagggggcauuuuaauauaucucugucgaguucauc

SEQ ID NO: 492 - RNA, 100 nt, Artificial Sequence
  Description of Artificial Sequence Synthetic polynucleotide
gauauugugacauaucucugcacucaucacuggcaugaugcaaaucuucuauaggauccacagggagggggcauuuuaacauaucucugcgagguucauc

SEQ ID NO: 493 - RNA, 100 nt, Artificial Sequence
  Description of Artificial Sequence Synthetic polynucleotide
gauauugugacauaucucugcacucaucacugucuugaugcaaaucuucuauaggauccacagggagggggcauuucaauauaucucugccucuuucauc

SEQ ID NO: 494 - RNA, 100 nt, Artificial Sequence
  Description of Artificial Sequence Synthetic polynucleotide
gauauugugacauaucucugcacucaucaccccuccgaugcaaaucuucuauaggauccacagggagggggcauucuaauauaucucugcguaccucauc

SEQ ID NO: 495 - RNA, 100 nt, Artificial Sequence
  Description of Artificial Sequence Synthetic polynucleotide
gauauugugacauaucucugcacucaucacugccucgaugcaaaucuucuauaggauccacagggagggggcaucuuaauauaucucuguugguuucauc

SEQ ID NO: 496 - RNA, 100 nt, Artificial Sequence
  Description of Artificial Sequence Synthetic polynucleotide
gauauugugacauaucucugcacucaucaccuugccgaugcaaaucuucuauaggauccacagggagggggcauuuuaauauaucucugcggaucucauc

SEQ ID NO: 497 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaucucuauuuauaucauc

SEQ ID NO: 498 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaucucuuuguacaucauc

SEQ ID NO: 499 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauaucucucauuuuaucauc

SEQ ID NO: 500 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaucucucuccacaucauc

SEQ ID NO: 501 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauaucucuucaaauaucauc

SEQ ID NO: 502 - RNA, 100 nt, Artificial Sequence
  Description of Artificial Sequence Synthetic polynucleotide
gauauugugacauaucucugcacucaucacacaaaagaugcaaaucuucuauagacuauucagggagggggcauuuuaacauaucucuuuuugcaucauc

SEQ ID NO: 503 - RNA, 100 nt, Artificial Sequence
  Description of Artificial Sequence Synthetic polynucleotide
gauauugugacauaucucugcacucaucacacaaaagaugcaaaucuucuauagcgcuuucagggagggggcauuucaauauaucucuuccgauaucauc

SEQ ID NO: 504 - RNA, 100 nt, Artificial Sequence
  Description of Artificial Sequence Synthetic polynucleotide
gauauugugacauaucucugcacucaucacacaaaagaugcaaaucuucuauagaaggaucagggagggggcauucuaauauaucucuauguauaucauc

SEQ ID NO: 505 - RNA, 100 nt, Artificial Sequence
  Description of Artificial Sequence Synthetic polynucleotide
gauauugugacauaucucugcacucaucacacaaaagaugcaaaucuucuauagucgggucagggagggggcaucuuaauauaucucuacguccaucauc

SEQ ID NO: 506 - RNA, 100 nt, Artificial Sequence
  Description of Artificial Sequence Synthetic polynucleotide
gauauugugacauaucucugcacucaucacacaaaagaugcaaaucuucuauaguacuuucagggagggggcauuuuaauauaucucuuucaccaucauc

SEQ ID NO: 507 - RNA, 100 nt, Artificial Sequence
  Description of Artificial Sequence Synthetic polynucleotide
gauauugugacauaucucugcacucaucacacaaaagaugcaaaucuucuauauagaaaacagggagggggcauuuuaacauaucucuuaugguaucauc

SEQ ID NO: 508 - RNA, 100 nt, Artificial Sequence
  Description of Artificial Sequence Synthetic polynucleotide
gauauugugacauaucucugcacucaucacacaaaagaugcaaaucuucuauaucuacaacagggagggggcauuucaauauaucucuuuuuccaucauc

SEQ ID NO: 509 - RNA, 100 nt, Artificial Sequence
  Description of Artificial Sequence Synthetic polynucleotide
gauauugugacauaucucugcacucaucacacaaaagaugcaaaucuucuauaaccuaaacagggagggggcauucuaauauaucucuuuccgcaucauc

SEQ ID NO: 510 - RNA, 100 nt, Artificial Sequence
  Description of Artificial Sequence Synthetic polynucleotide
gauauugugacauaucucugcacucaucacacaaaagaugcaaaucuucuauaacgcagacagggagggggcaucuuaauauaucucuucccgcaucauc

SEQ ID NO: 511 - RNA, 100 nt, Artificial Sequence
  Description of Artificial Sequence Synthetic polynucleotide
gauauugugacauaucucugcacucaucacacaaaagaugcaaaucuucuauacuuagaacagggagggggcauuuuaauauaucucuagugauaucauc

SEQ ID NO: 512 - RNA, 100 nt, Artificial Sequence
  Description of Artificial Sequence Synthetic polynucleotide
gauauugugacauaucucugcacucaucacacaaaagaugcaaaucuucuauucuccgcacagggagggggcauuuuaacauaucucuaucauuaucauc

SEQ ID NO: 513 - RNA, 100 nt, Artificial Sequence
  Description of Artificial Sequence Synthetic polynucleotide
gauauugugacauaucucugcacucaucacacaaaagaugcaaaucuucuaucuaccacacagggagggggcauuucaauauaucucuuaugauaucauc

SEQ ID NO: 514 - RNA, 100 nt, Artificial Sequence
  Description of Artificial Sequence Synthetic polynucleotide
gauauugugacauaucucugcacucaucacacaaaagaugcaaaucuucuauuaauuacacagggagggggcauucuaauauaucucuuccuccaucauc

SEQ ID NO: 515 - RNA, 100 nt, Artificial Sequence
  Description of Artificial Sequence Synthetic polynucleotide
gauauugugacauaucucugcacucaucacacaaaagaugcaaaucuucuauuauguacacagggagggggcaucuuaauauaucucuauggguaucauc

SEQ ID NO: 516 - RNA, 100 nt, Artificial Sequence
  Description of Artificial Sequence Synthetic polynucleotide
gauauugugacauaucucugcacucaucacacaaaagaugcaaaucuucuauuucgcgcacagggagggggcauuuuaauauaucucucaguccaucauc

SEQ ID NO: 517 - RNA, 100 nt, Artificial Sequence
  Description of Artificial Sequence Synthetic polynucleotide
gauauugugacauaucucugcacucaucacacaaaagaugcaaaucuucuaauugcaccacagggagggggcauuuuaacauaucucucuccauaucauc

SEQ ID NO: 518 - RNA, 100 nt, Artificial Sequence
  Description of Artificial Sequence Synthetic polynucleotide
gauauugugacauaucucugcacucaucacacaaaagaugcaaaucuucuagauuaaccacagggagggggcauuucaauauaucucucaugccaucauc

SEQ ID NO: 519 - RNA, 100 nt, Artificial Sequence
  Description of Artificial Sequence Synthetic polynucleotide
gauauugugacauaucucugcacucaucacacaaaagaugcaaaucuucuaguccuaccacagggagggggcauucuaauauaucucuuccuccaucauc

SEQ ID NO: 520 - RNA, 100 nt, Artificial Sequence
  Description of Artificial Sequence Synthetic polynucleotide
gauauugugacauaucucugcacucaucacacaaaagaugcaaaucuucuagacaaaccacagggagggggcaucuuaauauaucucuacagguaucauc

SEQ ID NO: 521 - RNA, 100 nt, Artificial Sequence
  Description of Artificial Sequence Synthetic polynucleotide
gauauugugacauaucucugcacucaucacacaaaagaugcaaaucuucuaggcuggccacagggagggggcauuuuaauauaucucuaccguuaucauc

SEQ ID NO: 522 - RNA, 100 nt, Artificial Sequence
  Description of Artificial Sequence Synthetic polynucleotide
gauauugugacauaucucugcacucaucacacaaaagaugcaaaucuucuugauucuccacagggagggggcauuuuaacauaucucucaccgcaucauc

SEQ ID NO: 523 - RNA, 100 nt, Artificial Sequence
  Description of Artificial Sequence Synthetic polynucleotide
gauauugugacauaucucugcacucaucacacaaaagaugcaaaucuucuuuacauuccacagggagggggcauuucaauauaucucuuaguauaucauc

SEQ ID NO: 524 - RNA, 100 nt, Artificial Sequence
  Description of Artificial Sequence Synthetic polynucleotide
gauauugugacauaucucugcacucaucacacaaaagaugcaaaucuucuuucaucuccacagggagggggcauucuaauauaucucuccucauaucauc

SEQ ID NO: 525 - RNA, 100 nt, Artificial Sequence
  Description of Artificial Sequence Synthetic polynucleotide
gauauugugacauaucucugcacucaucacacaaaagaugcaaaucuucuccaauuuccacagggagggggcaucuuaauauaucucucccgauaucauc

SEQ ID NO: 526 - RNA, 100 nt, Artificial Sequence
  Description of Artificial Sequence Synthetic polynucleotide
gauauugugacauaucucugcacucaucacacaaaagaugcaaaucuucuuuaaucuccacagggagggggcauuuuaauauaucucuuuccgcaucauc

SEQ ID NO: 527 - RNA, 100 nt, Artificial Sequence
  Description of Artificial Sequence Synthetic polynucleotide
gauauugugacauaucucugcacucaucacacaaaagaugcaaaucuucacuuucauccacagggagggggcauuuuaacauaucucucucguuaucauc

SEQ ID NO: 528 - RNA, 100 nt, Artificial Sequence
  Description of Artificial Sequence Synthetic polynucleotide
gauauugugacauaucucugcacucaucacacaaaagaugcaaaucuucguauccauccacagggagggggcauucuaauauaucucuuuccgcaucauc

SEQ ID NO: 529 - RNA, 100 nt, Artificial Sequence
  Description of Artificial Sequence Synthetic polynucleotide
gauauugugacauaucucugcacucaucacacaaaagaugcaaaucuucauucauauccacagggagggggcaucuuaauauaucucuuguuccaucauc

SEQ ID NO: 530 - RNA, 100 nt, Artificial Sequence
  Description of Artificial Sequence Synthetic polynucleotide
gauauugugacauaucucugcacucaucacacaaaagaugcaaaucuucacuuccauccacagggagggggcauuuuaauauaucucuuuaaauaucauc

SEQ ID NO: 531 - RNA, 100 nt, Artificial Sequence
  Description of Artificial Sequence Synthetic polynucleotide
gauauugugacauaucucugcacucaucacacaaaagaugcaaaucuugacaccgauccacagggagggggcauuucaauauaucucuaccaauaucauc

SEQ ID NO: 532 - RNA, 100 nt, Artificial Sequence
  Description of Artificial Sequence Synthetic polynucleotide
gauauugugacauaucucugcacucaucacacaaaagaugcaaaucuugaggucgauccacagggagggggcauucuaauauaucucuaguuacaucauc

SEQ ID NO: 533 - RNA, 100 nt, Artificial Sequence
  Description of Artificial Sequence Synthetic polynucleotide
gauauugugacauaucucugcacucaucacacaaaagaugcaaaucuuacucucgauccacagggagggggcaucuuaauauaucucucguuacaucauc

SEQ ID NO: 534 - RNA, 100 nt, Artificial Sequence
  Description of Artificial Sequence Synthetic polynucleotide
gauauugugacauaucucugcacucaucacacaaaagaugcaaaucuugcaaaugauccacagggagggggcauuuuaauauaucucucuugguaucauc

SEQ ID NO: 535 - RNA, 100 nt, Artificial Sequence
  Description of Artificial Sequence Synthetic polynucleotide
gauauugugacauaucucugcacucaucacacaaaagaugcaaaucucacuucggauccacagggagggggcauuuuaacauaucucuuuagauaucauc

SEQ ID NO: 536 - RNA, 100 nt, Artificial Sequence
  Description of Artificial Sequence Synthetic polynucleotide
gauauugugacauaucucugcacucaucacacaaaagaugcaaaucuggccuuggauccacagggagggggcauuucaauauaucucuuuugccaucauc

SEQ ID NO: 537 - RNA, 100 nt, Artificial Sequence
  Description of Artificial Sequence Synthetic polynucleotide
gauauugugacauaucucugcacucaucacacaaaagaugcaaaucucccuccggauccacagggagggggcauucuaauauaucucuaucgccaucauc

SEQ ID NO: 538 - RNA, 100 nt, Artificial Sequence
  Description of Artificial Sequence Synthetic polynucleotide
gauauugugacauaucucugcacucaucacacaaaagaugcaaaucuagacacggauccacagggagggggcaucuuaauauaucucuauuguuaucauc

SEQ ID NO: 539 - RNA, 100 nt, Artificial Sequence
  Description of Artificial Sequence Synthetic polynucleotide
gauauugugacauaucucugcacucaucacacaaaagaugcaaaucugucccuggauccacagggagggggcauuuuaauauaucucuuguaguaucauc

SEQ ID NO: 540 - RNA, 100 nt, Artificial Sequence
  Description of Artificial Sequence Synthetic polynucleotide
gauauugugacauaucucugcacucaucacacaaaagaugcaaaucgcguugaggauccacagggagggggcauuuuaacauaucucuaucccuaucauc

SEQ ID NO: 541 - RNA, 100 nt, Artificial Sequence
  Description of Artificial Sequence Synthetic polynucleotide
gauauugugacauaucucugcacucaucacacaaaagaugcaaaucccgcacaggauccacagggagggggcauuucaauauaucucuuccgauaucauc

SEQ ID NO: 542 - RNA, 100 nt, Artificial Sequence
  Description of Artificial Sequence Synthetic polynucleotide
gauauugugacauaucucugcacucaucacacaaaagaugcaaaucaggaagaggauccacagggagggggcauucuaauauaucucuuucaguaucauc

SEQ ID NO: 543 - RNA, 100 nt, Artificial Sequence
  Description of Artificial Sequence Synthetic polynucleotide
gauauugugacauaucucugcacucaucacacaaaagaugcaaaucaagcccaggauccacagggagggggcaucuuaauauaucucuaucaauaucauc

SEQ ID NO: 544 - RNA, 100 nt, Artificial Sequence
  Description of Artificial Sequence Synthetic polynucleotide
gauauugugacauaucucugcacucaucacacaaaagaugcaaaucacaaggaggauccacagggagggggcauuuuaauauaucucuauuuauaucauc

SEQ ID NO: 545 - RNA, 100 nt, Artificial Sequence
  Description of Artificial Sequence Synthetic polynucleotide
gauauugugacauaucucugcacucaucacacaaaagaugcaaaugagagcuaggauccacagggagggggcauuuuaacauaucucuucauauaucauc

SEQ ID NO: 546 - RNA, 100 nt, Artificial Sequence
  Description of Artificial Sequence Synthetic polynucleotide
gauauugugacauaucucugcacucaucacacaaaagaugcaaauggcgguuaggauccacagggagggggcauuucaauauaucucuuuuuccaucauc

SEQ ID NO: 547 - RNA, 100 nt, Artificial Sequence
  Description of Artificial Sequence Synthetic polynucleotide
gauauugugacauaucucugcacucaucacacaaaagaugcaaauggagguuaggauccacagggagggggcauucuaauauaucucucuaaauaucauc

SEQ ID NO: 548 - RNA, 100 nt, Artificial Sequence
  Description of Artificial Sequence Synthetic polynucleotide
gauauugugacauaucucugcacucaucacacaaaagaugcaaauaccacuuaggauccacagggagggggcaucuuaauauaucucuauccccaucauc

SEQ ID NO: 549 - RNA, 100 nt, Artificial Sequence
  Description of Artificial Sequence Synthetic polynucleotide
gauauugugacauaucucugcacucaucacacaaaagaugcaaagcaggcauaggauccacagggagggggcauuucaauauaucucuccaguuaucauc

SEQ ID NO: 550 - RNA, 100 nt, Artificial Sequence
  Description of Artificial Sequence Synthetic polynucleotide
gauauugugacauaucucugcacucaucacacaaaagaugcaaagagagcauaggauccacagggagggggcauucuaauauaucucuccugguaucauc

SEQ ID NO: 551 - RNA, 100 nt, Artificial Sequence
  Description of Artificial Sequence Synthetic polynucleotide
gauauugugacauaucucugcacucaucacacaaaagaugcaaacgaggaauaggauccacagggagggggcaucuuaauauaucucucaccccaucauc

SEQ ID NO: 552 - RNA, 100 nt, Artificial Sequence
  Description of Artificial Sequence Synthetic polynucleotide
gauauugugacauaucucugcacucaucacacaaaagaugcaaaagccgaauaggauccacagggagggggcauuuuaauauaucucucaguauaucauc

SEQ ID NO: 553 - RNA, 100 nt, Artificial Sequence
  Description of Artificial Sequence Synthetic polynucleotide
gauauugugacauaucucugcacucaucacacaaaagaugcaacaacgauauaggauccacagggagggggcauuuuaacauaucucuauguucaucauc

SEQ ID NO: 554 - RNA, 100 nt, Artificial Sequence
  Description of Artificial Sequence Synthetic polynucleotide
gauauugugacauaucucugcacucaucacacaaaagaugcaaccaacguauaggauccacagggagggggcauuucaauauaucucucccuucaucauc

SEQ ID NO: 555 - RNA, 100 nt, Artificial Sequence
  Description of Artificial Sequence Synthetic polynucleotide
gauauugugacauaucucugcacucaucacacaaaagaugcaaugacaauauaggauccacagggagggggcauucuaauauaucucuccaaauaucauc

SEQ ID NO: 556 - RNA, 100 nt, Artificial Sequence
  Description of Artificial Sequence Synthetic polynucleotide
gauauugugacauaucucugcacucaucacacaaaagaugcaacugguauauaggauccacagggagggggcaucuuaauauaucucucgccgcaucauc

SEQ ID NO: 557 - RNA, 100 nt, Artificial Sequence
  Description of Artificial Sequence Synthetic polynucleotide
gauauugugacauaucucugcacucaucacacaaaagaugcaaugacgauauaggauccacagggagggggcauuuuaauauaucucuuauaauaucauc

SEQ ID NO: 558 - RNA, 100 nt, Artificial Sequence
  Description of Artificial Sequence Synthetic polynucleotide
gauauugugacauaucucugcacucaucacacaaaagaugcauaagggcuauaggauccacagggagggggcauuuuaacauaucucuuuuaauaucauc

SEQ ID NO: 559 - RNA, 100 nt, Artificial Sequence
  Description of Artificial Sequence Synthetic polynucleotide
gauauugugacauaucucugcacucaucacacaaaagaugcauucauccuauaggauccacagggagggggcauucuaauauaucucuccaaguaucauc

SEQ ID NO: 560 - RNA, 100 nt, Artificial Sequence
  Description of Artificial Sequence Synthetic polynucleotide
gauauugugacauaucucugcacucaucacacaaaagaugcaccgaaacuauaggauccacagggagggggcaucuuaauauaucucuagugauaucauc

SEQ ID NO: 561 - RNA, 100 nt, Artificial Sequence
  Description of Artificial Sequence Synthetic polynucleotide
gauauugugacauaucucugcacucaucacacaaaagaugcauggagacuauaggauccacagggagggggcauuuuaauauaucucuuauuuuaucauc

SEQ ID NO: 562 - RNA, 100 nt, Artificial Sequence
  Description of Artificial Sequence Synthetic polynucleotide
gauauugugacauaucucugcacucaucacacaaaagaugccccaugucuauaggauccacagggagggggcauuuuaacauaucucuuucccuaucauc

SEQ ID NO: 563 - RNA, 100 nt, Artificial Sequence
  Description of Artificial Sequence Synthetic polynucleotide
gauauugugacauaucucugcacucaucacacaaaagaugccauaacucuauaggauccacagggagggggcauucuaauauaucucuaccuuuaucauc

SEQ ID NO: 564 - RNA, 100 nt, Artificial Sequence
  Description of Artificial Sequence Synthetic polynucleotide
gauauugugacauaucucugcacucaucacacaaaagaugccuacagucuauaggauccacagggagggggcaucuuaauauaucucuauuuacaucauc

SEQ ID NO: 565 - RNA, 100 nt, Artificial Sequence
  Description of Artificial Sequence Synthetic polynucleotide
gauauugugacauaucucugcacucaucacacaaaagaugcuugccgucuauaggauccacagggagggggcauuuuaauauaucucuauuaauaucauc

SEQ ID NO: 566 - RNA, 100 nt, Artificial Sequence
  Description of Artificial Sequence Synthetic polynucleotide
gauauugugacauaucucugcacucaucacacaaaagaugauuggguucuauaggauccacagggagggggcauuucaauauaucucuuccaccaucauc

SEQ ID NO: 567 - RNA, 100 nt, Artificial Sequence
  Description of Artificial Sequence Synthetic polynucleotide
gauauugugacauaucucugcacucaucacacaaaagaugguaugguucuauaggauccacagggagggggcauucuaauauaucucuucuuacaucauc

SEQ ID NO: 568 - RNA, 100 nt, Artificial Sequence
  Description of Artificial Sequence Synthetic polynucleotide
gauauugugacauaucucugcacucaucacacaaaagaugaccagguucuauaggauccacagggagggggcaucuuaauauaucucuuuugguaucauc

SEQ ID NO: 569 - RNA, 100 nt, Artificial Sequence
  Description of Artificial Sequence Synthetic polynucleotide
gauauugugacauaucucugcacucaucacacaaaagaugagcgaauucuauaggauccacagggagggggcauuuuaauauaucucuuuuuacaucauc

SEQ ID NO: 570 - RNA, 100 nt, Artificial Sequence
  Description of Artificial Sequence Synthetic polynucleotide
gauauugugacauaucucugcacucaucacacaaaagauaauggacuucuauaggauccacagggagggggcauuuuaacauaucucuuauaauaucauc

SEQ ID NO: 571 - RNA, 100 nt, Artificial Sequence
  Description of Artificial Sequence Synthetic polynucleotide
gauauugugacauaucucugcacucaucacacaaaagauaucgugcuucuauaggauccacagggagggggcauuucaauauaucucuauucauaucauc

SEQ ID NO: 572 - RNA, 100 nt, Artificial Sequence
  Description of Artificial Sequence Synthetic polynucleotide
gauauugugacauaucucugcacucaucacacaaaagauuucgugcuucuauaggauccacagggagggggcauucuaauauaucucuacccgcaucauc

SEQ ID NO: 573 - RNA, 100 nt, Artificial Sequence
  Description of Artificial Sequence Synthetic polynucleotide
gauauugugacauaucucugcacucaucacacaaaagauauccgacuucuauaggauccacagggagggggcaucuuaauauaucucucaccacaucauc

SEQ ID NO: 574 - RNA, 100 nt, Artificial Sequence
  Description of Artificial Sequence Synthetic polynucleotide
gauauugugacauaucucugcacucaucacacaaaagauuccuaccuucuauaggauccacagggagggggcauuuuaauauaucucucucgguaucauc

SEQ ID NO: 575 - RNA, 100 nt, Artificial Sequence
  Description of Artificial Sequence Synthetic polynucleotide
gauauugugacauaucucugcacucaucacacaaaagaccauucucuucuauaggauccacagggagggggcauuuuaacauaucucuuccccuaucauc

SEQ ID NO: 576 - RNA, 100 nt, Artificial Sequence
  Description of Artificial Sequence Synthetic polynucleotide
gauauugugacauaucucugcacucaucacacaaaagaaaaaccucuucuauaggauccacagggagggggcauuucaauauaucucuucaaauaucauc

SEQ ID NO: 577 - RNA, 100 nt, Artificial Sequence
  Description of Artificial Sequence Synthetic polynucleotide
gauauugugacauaucucugcacucaucacacaaaagaccguuuucuucuauaggauccacagggagggggcauucuaauauaucucuccuuauaucauc

SEQ ID NO: 578 - RNA, 100 nt, Artificial Sequence
  Description of Artificial Sequence Synthetic polynucleotide
gauauugugacauaucucugcacucaucacacaaaagagaguccucuucuauaggauccacagggagggggcaucuuaauauaucucuaucuccaucauc

SEQ ID NO: 579 - RNA, 100 nt, Artificial Sequence
  Description of Artificial Sequence Synthetic polynucleotide
gauauugugacauaucucugcacucaucacacaaaagcuugauaucuucuauaggauccacagggagggggcauuuuaacauaucucuccuugcaucauc

SEQ ID NO: 580 - RNA, 100 nt, Artificial Sequence
  Description of Artificial Sequence Synthetic polynucleotide
gauauugugacauaucucugcacucaucacacaaaagcgaacuaucuucuauaggauccacagggagggggcauuucaauauaucucucccuccaucauc

SEQ ID NO: 581 - RNA, 100 nt, Artificial Sequence
  Description of Artificial Sequence Synthetic polynucleotide
gauauugugacauaucucugcacucaucacacaaaagcuauccaucuucuauaggauccacagggagggggcauucuaauauaucucuucuugcaucauc

SEQ ID NO: 582 - RNA, 100 nt, Artificial Sequence
  Description of Artificial Sequence Synthetic polynucleotide
gauauugugacauaucucugcacucaucacacaaaagucaguuaucuucuauaggauccacagggagggggcaucuuaauauaucucucgcaguaucauc

SEQ ID NO: 583 - RNA, 100 nt, Artificial Sequence
  Description of Artificial Sequence Synthetic polynucleotide
gauauugugacauaucucugcacucaucacacaaaaguuugacaucuucuauaggauccacagggagggggcauuuuaauauaucucuauuauuaucauc

SEQ ID NO: 584 - RNA, 100 nt, Artificial Sequence
  Description of Artificial Sequence Synthetic polynucleotide
gauauugugacauaucucugcacucaucacacaaaacuaucuaaucuucuauaggauccacagggagggggcauuuuaacauaucucuccuuccaucauc

SEQ ID NO: 585 - RNA, 100 nt, Artificial Sequence
  Description of Artificial Sequence Synthetic polynucleotide
gauauugugacauaucucugcacucaucacacaaaacagaauaaucuucuauaggauccacagggagggggcauuucaauauaucucuuucgccaucauc

SEQ ID NO: 586 - RNA, 100 nt, Artificial Sequence
  Description of Artificial Sequence Synthetic polynucleotide
gauauugugacauaucucugcacucaucacacaaaauccaauaaucuucuauaggauccacagggagggggcauucuaauauaucucuuccuccaucauc

SEQ ID NO: 587 - RNA, 100 nt, Artificial Sequence
  Description of Artificial Sequence Synthetic polynucleotide
gauauugugacauaucucugcacucaucacacaaaacccaauaaucuucuauaggauccacagggagggggcaucuuaauauaucucuacagguaucauc

SEQ ID NO: 588 - RNA, 100 nt, Artificial Sequence
  Description of Artificial Sequence Synthetic polynucleotide
gauauugugacauaucucugcacucaucacacaaaacuuaucaaucuucuauaggauccacagggagggggcauuuuaauauaucucuuucgguaucauc

SEQ ID NO: 589 - RNA, 100 nt, Artificial Sequence
  Description of Artificial Sequence Synthetic polynucleotide
gauauugugacauaucucugcacucaucacacaaauuuaaaaaaucuucuauaggauccacagggagggggcauuuuaacauaucucuacccacaucauc

SEQ ID NO: 590 - RNA, 100 nt, Artificial Sequence
  Description of Artificial Sequence Synthetic polynucleotide
gauauugugacauaucucugcacucaucacacaaacaccgaaaaucuucuauaggauccacagggagggggcauuucaauauaucucuuaguccaucauc

SEQ ID NO: 591 - RNA, 100 nt, Artificial Sequence
  Description of Artificial Sequence Synthetic polynucleotide
gauauugugacauaucucugcacucaucacacaaauaaauaaaaucuucuauaggauccacagggagggggcaucuuaauauaucucucaugccaucauc

SEQ ID NO: 592 - RNA, 100 nt, Artificial Sequence
  Description of Artificial Sequence Synthetic polynucleotide
gauauugugacauaucucugcacucaucacacaaacuuacgaaaucuucuauaggauccacagggagggggcauuuuaauauaucucuccguuuaucauc

SEQ ID NO: 593 - RNA, 100 nt, Artificial Sequence
  Description of Artificial Sequence Synthetic polynucleotide
gauauugugacauaucucugcacucaucacacaacccucccaaaucuucuauaggauccacagggagggggcauuuuaacauaucucucgugauaucauc

SEQ ID NO: 594 - RNA, 100 nt, Artificial Sequence
  Description of Artificial Sequence Synthetic polynucleotide
gauauugugacauaucucugcacucaucacacaaccuucccaaaucuucuauaggauccacagggagggggcauuucaauauaucucuucuugcaucauc

SEQ ID NO: 595 - RNA, 100 nt, Artificial Sequence
  Description of Artificial Sequence Synthetic polynucleotide
gauauugugacauaucucugcacucaucacacaauuccaacaaaucuucuauaggauccacagggagggggcauucuaauauaucucuauccauaucauc

SEQ ID NO: 596 - RNA, 100 nt, Artificial Sequence
  Description of Artificial Sequence Synthetic polynucleotide
gauauugugacauaucucugcacucaucacacaacgcgaccaaaucuucuauaggauccacagggagggggcaucuuaauauaucucuuaugauaucauc

SEQ ID NO: 597 - RNA, 100 nt, Artificial Sequence
  Description of Artificial Sequence Synthetic polynucleotide
gauauugugacauaucucugcacucaucacacaaccacaucaaaucuucuauaggauccacagggagggggcauuuuaauauaucucuccuguuaucauc

SEQ ID NO: 598 - RNA, 100 nt, Artificial Sequence
  Description of Artificial Sequence Synthetic polynucleotide
gauauugugacauaucucugcacucaucacacacacauggcaaaucuucuauaggauccacagggagggggcauuuuaacauaucucuccggguaucauc

SEQ ID NO: 599 - RNA, 100 nt, Artificial Sequence
  Description of Artificial Sequence Synthetic polynucleotide
gauauugugacauaucucugcacucaucacacacuguuagcaaaucuucuauaggauccacagggagggggcauuucaauauaucucuauccgcaucauc

SEQ ID NO: 600 - RNA, 100 nt, Artificial Sequence
  Description of Artificial Sequence Synthetic polynucleotide
gauauugugacauaucucugcacucaucacacacuaucagcaaaucuucuauaggauccacagggagggggcauucuaauauaucucucaccgcaucauc

SEQ ID NO: 601 - RNA, 100 nt, Artificial Sequence
  Description of Artificial Sequence Synthetic polynucleotide
gauauugugacauaucucugcacucaucacacacuucaagcaaaucuucuauaggauccacagggagggggcaucuuaauauaucucuccccgcaucauc

SEQ ID NO: 602 - RNA, 100 nt, Artificial Sequence
  Description of Artificial Sequence Synthetic polynucleotide
gauauugugacauaucucugcacucaucacacacaucgcgcaaaucuucuauaggauccacagggagggggcauuuuaauauaucucuuucgguaucauc

SEQ ID NO: 603 - RNA, 100 nt, Artificial Sequence
  Description of Artificial Sequence Synthetic polynucleotide
gauauugugacauaucucugcacucaucacacugcgauugcaaaucuucuauaggauccacagggagggggcauuuuaacauaucucuuuccgcaucauc

SEQ ID NO: 604 - RNA, 100 nt, Artificial Sequence
  Description of Artificial Sequence Synthetic polynucleotide
gauauugugacauaucucugcacucaucacacugucucugcaaaucuucuauaggauccacagggagggggcauuucaauauaucucuccuugcaucauc

SEQ ID NO: 605 - RNA, 100 nt, Artificial Sequence
  Description of Artificial Sequence Synthetic polynucleotide
gauauugugacauaucucugcacucaucacacccaguuugcaaaucuucuauaggauccacagggagggggcaucuuaauauaucucuucgguuaucauc

SEQ ID NO: 606 - RNA, 100 nt, Artificial Sequence
  Description of Artificial Sequence Synthetic polynucleotide
gauauugugacauaucucugcacucaucacacccucacugcaaaucuucuauaggauccacagggagggggcauuuuaauauaucucuauuuuuaucauc

SEQ ID NO: 607 - RNA, 100 nt, Artificial Sequence
  Description of Artificial Sequence Synthetic polynucleotide
gauauugugacauaucucugcacucaucacagccaucaugcaaaucuucuauaggauccacagggagggggcauuuuaacauaucucucuguccaucauc

SEQ ID NO: 608 - RNA, 100 nt, Artificial Sequence
  Description of Artificial Sequence Synthetic polynucleotide
gauauugugacauaucucugcacucaucacaaauacuaugcaaaucuucuauaggauccacagggagggggcauuucaauauaucucuuucaguaucauc

SEQ ID NO: 609 - RNA, 100 nt, Artificial Sequence
  Description of Artificial Sequence Synthetic polynucleotide
gauauugugacauaucucugcacucaucacaacucauaugcaaaucuucuauaggauccacagggagggggcauucuaauauaucucuauuuuuaucauc

SEQ ID NO: 610 - RNA, 100 nt, Artificial Sequence
  Description of Artificial Sequence Synthetic polynucleotide
gauauugugacauaucucugcacucaucacaauggcaaugcaaaucuucuauaggauccacagggagggggcaucuuaauauaucucucgccccaucauc

SEQ ID NO: 611 - RNA, 100 nt, Artificial Sequence
  Description of Artificial Sequence Synthetic polynucleotide
gauauugugacauaucucugcacucaucacaaaaucaaugcaaaucuucuauaggauccacagggagggggcauuuuaauauaucucucuuaguaucauc

SEQ ID NO: 612 - RNA, 100 nt, Artificial Sequence
  Description of Artificial Sequence Synthetic polynucleotide
gauauugugacauaucucugcacucaucacuuguccgaugcaaaucuucuauaggauccacagggagggggcauuuuaacauaucucuaguaguaucauc

SEQ ID NO: 613 - RNA, 100 nt, Artificial Sequence
  Description of Artificial Sequence Synthetic polynucleotide
gauauugugacauaucucugcacucaucacuauaccgaugcaaaucuucuauaggauccacagggagggggcauuucaauauaucucucuccgcaucauc

SEQ ID NO: 614 - RNA, 100 nt, Artificial Sequence
  Description of Artificial Sequence Synthetic polynucleotide
gauauugugacauaucucugcacucaucacuauuucgaugcaaaucuucuauaggauccacagggagggggcauucuaauauaucucuucccacaucauc

SEQ ID NO: 615 - RNA, 100 nt, Artificial Sequence
  Description of Artificial Sequence Synthetic polynucleotide
gauauugugacauaucucugcacucaucacugauaugaugcaaaucuucuauaggauccacagggagggggcaucuuaauauaucucuauugccaucauc

SEQ ID NO: 616 - RNA, 100 nt, Artificial Sequence
  Description of Artificial Sequence Synthetic polynucleotide
gauauugugacauaucucugcacucaucacuauuacgaugcaaaucuucuauaggauccacagggagggggcauuuuaauauaucucuaucaccaucauc

SEQ ID NO: 617 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaucuccuucggaaucauc

SEQ ID NO: 618 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaucucaucuaaaaucauc

SEQ ID NO: 619 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauaucucccacaaaaucauc

SEQ ID NO: 620 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaucucggugagaaucauc

SEQ ID NO: 621 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauaucuccccuggaaucauc

SEQ ID NO: 622 - RNA, 100 nt, Artificial Sequence
  Description of Artificial Sequence Synthetic polynucleotide
gauauugugacauaucucugcacucaucacacaaaagaugcaaaucuucuauagcaaauucagggagggggcauuuuaacauaucucauuucgaaucauc

SEQ ID NO: 623 - RNA, 100 nt, Artificial Sequence
  Description of Artificial Sequence Synthetic polynucleotide
gauauugugacauaucucugcacucaucacacaaaagaugcaaaucuucuauagugcagucagggagggggcauuucaauauaucucaacaagaaucauc

SEQ ID NO: 624 - RNA, 100 nt, Artificial Sequence
  Description of Artificial Sequence Synthetic polynucleotide
gauauugugacauaucucugcacucaucacacaaaagaugcaaaucuucuauagacugaucagggagggggcauucuaauauaucucaucguaaaucauc

SEQ ID NO: 625 - RNA, 100 nt, Artificial Sequence
  Description of Artificial Sequence Synthetic polynucleotide
gauauugugacauaucucugcacucaucacacaaaagaugcaaaucuucuauagaagugucagggagggggcaucuuaauauaucuccaguucaaucauc

SEQ ID NO: 626 - RNA, 100 nt, Artificial Sequence
  Description of Artificial Sequence Synthetic polynucleotide
gauauugugacauaucucugcacucaucacacaaaagaugcaaaucuucuauagccggaucagggagggggcauuuuaauauaucuccucuggaaucauc

SEQ ID NO: 627 - RNA, 100 nt, Artificial Sequence
  Description of Artificial Sequence Synthetic polynucleotide
gauauugugacauaucucugcacucaucacacaaaagaugcaaaucuucuauaaucaaaacagggagggggcauuuuaacauaucucccucaaaaucauc

SEQ ID NO: 628 - RNA, 100 nt, Artificial Sequence
  Description of Artificial Sequence Synthetic polynucleotide
gauauugugacauaucucugcacucaucacacaaaagaugcaaaucuucuauaacugcaacagggagggggcauuucaauauaucucgccuggaaucauc

SEQ ID NO: 629 - RNA, 100 nt, Artificial Sequence
  Description of Artificial Sequence Synthetic polynucleotide
gauauugugacauaucucugcacucaucacacaaaagaugcaaaucuucuauaaaguggacagggagggggcauucuaauauaucucguuuaaaaucauc

SEQ ID NO: 630 - RNA, 100 nt, Artificial Sequence
  Description of Artificial Sequence Synthetic polynucleotide
gauauugugacauaucucugcacucaucacacaaaagaugcaaaucuucuauauuuauaacagggagggggcaucuuaauauaucucgcaggaaaucauc

SEQ ID NO: 631 - RNA, 100 nt, Artificial Sequence
  Description of Artificial Sequence Synthetic polynucleotide
gauauugugacauaucucugcacucaucacacaaaagaugcaaaucuucuauauugcgaacagggagggggcauuuuaauauaucucccccggaaucauc

SEQ ID NO: 632 - RNA, 100 nt, Artificial Sequence
  Description of Artificial Sequence Synthetic polynucleotide
gauauugugacauaucucugcacucaucacacaaaagaugcaaaucuucuauuaaaaacacagggagggggcauuuuaacauaucucgguccgaaucauc

SEQ ID NO: 633 - RNA, 100 nt, Artificial Sequence
  Description of Artificial Sequence Synthetic polynucleotide
gauauugugacauaucucugcacucaucacacaaaagaugcaaaucuucuauuacacgcacagggagggggcauuucaauauaucucccgcagaaucauc

SEQ ID NO: 634 - RNA, 100 nt, Artificial Sequence
  Description of Artificial Sequence Synthetic polynucleotide
gauauugugacauaucucugcacucaucacacaaaagaugcaaaucuucuaucuauagcacagggagggggcauucuaauauaucucgguuucaaucauc

SEQ ID NO: 635 - RNA, 100 nt, Artificial Sequence
  Description of Artificial Sequence Synthetic polynucleotide
gauauugugacauaucucugcacucaucacacaaaagaugcaaaucuucuaucccuggcacagggagggggcaucuuaauauaucucgauugaaaucauc

SEQ ID NO: 636 - RNA, 100 nt, Artificial Sequence
  Description of Artificial Sequence Synthetic polynucleotide
gauauugugacauaucucugcacucaucacacaaaagaugcaaaucuucuauuccuuacacagggagggggcauuuuaauauaucucauuuagaaucauc

SEQ ID NO: 637 - RNA, 100 nt, Artificial Sequence
  Description of Artificial Sequence Synthetic polynucleotide
gauauugugacauaucucugcacucaucacacaaaagaugcaaaucuucuaaacguaccacagggagggggcauuuuaacauaucucgcguuaaaucauc

SEQ ID NO: 638 - RNA, 100 nt, Artificial Sequence
  Description of Artificial Sequence Synthetic polynucleotide
gauauugugacauaucucugcacucaucacacaaaagaugcaaaucuucuacaaaaaccacagggagggggcauuucaauauaucucccgcccaaucauc

SEQ ID NO: 639 - RNA, 100 nt, Artificial Sequence
  Description of Artificial Sequence Synthetic polynucleotide
gauauugugacauaucucugcacucaucacacaaaagaugcaaaucuucuacucgcgccacagggagggggcauucuaauauaucucgcccgaaaucauc

SEQ ID NO: 640 - RNA, 100 nt, Artificial Sequence
  Description of Artificial Sequence Synthetic polynucleotide
gauauugugacauaucucugcacucaucacacaaaagaugcaaaucuucuaaugcacccacagggagggggcaucuuaauauaucucguuuuaaaucauc

SEQ ID NO: 641 - RNA, 100 nt, Artificial Sequence
  Description of Artificial Sequence Synthetic polynucleotide
gauauugugacauaucucugcacucaucacacaaaagaugcaaaucuucuacauuccccacagggagggggcauuuuaauauaucucauaccgaaucauc

SEQ ID NO: 642 - RNA, 100 nt, Artificial Sequence
  Description of Artificial Sequence Synthetic polynucleotide
gauauugugacauaucucugcacucaucacacaaaagaugcaaaucuucuccuuuuuccacagggagggggcauuuuaacauaucucauuguaaaucauc

SEQ ID NO: 643 - RNA, 100 nt, Artificial Sequence
  Description of Artificial Sequence Synthetic polynucleotide
gauauugugacauaucucugcacucaucacacaaaagaugcaaaucuucuugcauuuccacagggagggggcauuucaauauaucuccuauggaaucauc

SEQ ID NO: 644 - RNA, 100 nt, Artificial Sequence
  Description of Artificial Sequence Synthetic polynucleotide
gauauugugacauaucucugcacucaucacacaaaagaugcaaaucuucuccuccuuccacagggagggggcauucuaauauaucucgcguugaaucauc

SEQ ID NO: 645 - RNA, 100 nt, Artificial Sequence
  Description of Artificial Sequence Synthetic polynucleotide
gauauugugacauaucucugcacucaucacacaaaagaugcaaaucuucucucuacuccacagggagggggcaucuuaauauaucucgacgaaaaucauc

SEQ ID NO: 646 - RNA, 100 nt, Artificial Sequence
  Description of Artificial Sequence Synthetic polynucleotide
gauauugugacauaucucugcacucaucacacaaaagaugcaaaucuucucccuuuuccacagggagggggcauuuuaauauaucuccuauggaaucauc

SEQ ID NO: 647 - RNA, 100 nt, Artificial Sequence
  Description of Artificial Sequence Synthetic polynucleotide
gauauugugacauaucucugcacucaucacacaaaagaugcaaaucuucaaucuuauccacagggagggggcauuuuaacauaucucccgucgaaucauc

SEQ ID NO: 648 - RNA, 100 nt, Artificial Sequence
  Description of Artificial Sequence Synthetic polynucleotide
gauauugugacauaucucugcacucaucacacaaaagaugcaaaucuuccuccgcauccacagggagggggcauuucaauauaucucguauggaaucauc

SEQ ID NO: 649 - RNA, 100 nt, Artificial Sequence
  Description of Artificial Sequence Synthetic polynucleotide
gauauugugacauaucucugcacucaucacacaaaagaugcaaaucuucgauucaauccacagggagggggcauucuaauauaucuccauuucaaucauc

SEQ ID NO: 650 - RNA, 100 nt, Artificial Sequence
  Description of Artificial Sequence Synthetic polynucleotide
gauauugugacauaucucugcacucaucacacaaaagaugcaaaucuucgucgcuauccacagggagggggcaucuuaauauaucuccuuucgaaucauc

SEQ ID NO: 651 - RNA, 100 nt, Artificial Sequence
  Description of Artificial Sequence Synthetic polynucleotide
gauauugugacauaucucugcacucaucacacaaaagaugcaaaucuuccgcaucauccacagggagggggcauuuuaauauaucucacuguaaaucauc

SEQ ID NO: 652 - RNA, 100 nt, Artificial Sequence
  Description of Artificial Sequence Synthetic polynucleotide
gauauugugacauaucucugcacucaucacacaaaagaugcaaaucuuaccugcgauccacagggagggggcauuucaauauaucucauuuaaaaucauc

SEQ ID NO: 653 - RNA, 100 nt, Artificial Sequence
  Description of Artificial Sequence Synthetic polynucleotide
gauauugugacauaucucugcacucaucacacaaaagaugcaaaucuuacggccgauccacagggagggggcauucuaauauaucucauucccaaucauc

SEQ ID NO: 654 - RNA, 100 nt, Artificial Sequence
  Description of Artificial Sequence Synthetic polynucleotide
gauauugugacauaucucugcacucaucacacaaaagaugcaaaucuugguaucgauccacagggagggggcaucuuaauauaucucgucgggaaucauc

SEQ ID NO: 655 - RNA, 100 nt, Artificial Sequence
  Description of Artificial Sequence Synthetic polynucleotide
gauauugugacauaucucugcacucaucacacaaaagaugcaaaucuuaacgcugauccacagggagggggcauuuuaauauaucucaaccggaaucauc

SEQ ID NO: 656 - RNA, 100 nt, Artificial Sequence
  Description of Artificial Sequence Synthetic polynucleotide
gauauugugacauaucucugcacucaucacacaaaagaugcaaaucucaggacggauccacagggagggggcauuuuaacauaucucacguugaaucauc

SEQ ID NO: 657 - RNA, 100 nt, Artificial Sequence
  Description of Artificial Sequence Synthetic polynucleotide
gauauugugacauaucucugcacucaucacacaaaagaugcaaaucuauaagcggauccacagggagggggcauuucaauauaucucauuuucaaucauc

SEQ ID NO: 658 - RNA, 100 nt, Artificial Sequence
  Description of Artificial Sequence Synthetic polynucleotide
gauauugugacauaucucugcacucaucacacaaaagaugcaaaucucaucguggauccacagggagggggcaucuuaauauaucucccguagaaucauc

SEQ ID NO: 659 - RNA, 100 nt, Artificial Sequence
  Description of Artificial Sequence Synthetic polynucleotide
gauauugugacauaucucugcacucaucacacaaaagaugcaaaucuaauuacggauccacagggagggggcauuuuaauauaucuccaucgaaaucauc

SEQ ID NO: 660 - RNA, 100 nt, Artificial Sequence
  Description of Artificial Sequence Synthetic polynucleotide
gauauugugacauaucucugcacucaucacacaaaagaugcaaaucggauuaaggauccacagggagggggcauuuuaacauaucucaucccaaaucauc

SEQ ID NO: 661 - RNA, 100 nt, Artificial Sequence
  Description of Artificial Sequence Synthetic polynucleotide
gauauugugacauaucucugcacucaucacacaaaagaugcaaaucccgcgcaggauccacagggagggggcauuucaauauaucucgcucgaaaucauc

SEQ ID NO: 662 - RNA, 100 nt, Artificial Sequence
  Description of Artificial Sequence Synthetic polynucleotide
gauauugugacauaucucugcacucaucacacaaaagaugcaaauccuggagaggauccacagggagggggcauucuaauauaucuccuuuuaaaucauc

SEQ ID NO: 663 - RNA, 100 nt, Artificial Sequence
  Description of Artificial Sequence Synthetic polynucleotide
gauauugugacauaucucugcacucaucacacaaaagaugcaaaucccagucaggauccacagggagggggcaucuuaauauaucuccuugggaaucauc

SEQ ID NO: 664 - RNA, 100 nt, Artificial Sequence
  Description of Artificial Sequence Synthetic polynucleotide
gauauugugacauaucucugcacucaucacacaaaagaugcaaaucgggaacaggauccacagggagggggcauuuuaauauaucucaacccgaaucauc

SEQ ID NO: 665 - RNA, 100 nt, Artificial Sequence
  Description of Artificial Sequence Synthetic polynucleotide
gauauugugacauaucucugcacucaucacacaaaagaugcaaauaccuuuuaggauccacagggagggggcauuuuaacauaucucacucagaaucauc

SEQ ID NO: 666 - RNA, 100 nt, Artificial Sequence
  Description of Artificial Sequence Synthetic polynucleotide
gauauugugacauaucucugcacucaucacacaaaagaugcaaauaguauuuaggauccacagggagggggcauuucaauauaucucaaguaaaaucauc

SEQ ID NO: 667 - RNA, 100 nt, Artificial Sequence
  Description of Artificial Sequence Synthetic polynucleotide
gauauugugacauaucucugcacucaucacacaaaagaugcaaauggacacuaggauccacagggagggggcauucuaauauaucucaaucgaaaucauc

SEQ ID NO: 668 - RNA, 100 nt, Artificial Sequence
  Description of Artificial Sequence Synthetic polynucleotide
gauauugugacauaucucugcacucaucacacaaaagaugcaaauaagauuuaggauccacagggagggggcaucuuaauauaucucaaguucaaucauc

SEQ ID NO: 669 - RNA, 100 nt, Artificial Sequence
  Description of Artificial Sequence Synthetic polynucleotide
gauauugugacauaucucugcacucaucacacaaaagaugcaaauauaguuuaggauccacagggagggggcauuuuaauauaucucgaugucaaucauc

SEQ ID NO: 670 - RNA, 100 nt, Artificial Sequence
  Description of Artificial Sequence Synthetic polynucleotide
gauauugugacauaucucugcacucaucacacaaaagaugcaaacaggacauaggauccacagggagggggcauuuuaacauaucuccccucgaaucauc

SEQ ID NO: 671 - RNA, 100 nt, Artificial Sequence
  Description of Artificial Sequence Synthetic polynucleotide
gauauugugacauaucucugcacucaucacacaaaagaugcaaacaggugauaggauccacagggagggggcauucuaauauaucucgguuucaaucauc

SEQ ID NO: 672 - RNA, 100 nt, Artificial Sequence
  Description of Artificial Sequence Synthetic polynucleotide
gauauugugacauaucucugcacucaucacacaaaagaugcaaacaacccauaggauccacagggagggggcaucuuaauauaucuccaccaaaaucauc

SEQ ID NO: 673 - RNA, 100 nt, Artificial Sequence
  Description of Artificial Sequence Synthetic polynucleotide
gauauugugacauaucucugcacucaucacacaaaagaugcaaaggguaaauaggauccacagggagggggcauuuuaauauaucucaccaugaaucauc

SEQ ID NO: 674 - RNA, 100 nt, Artificial Sequence
  Description of Artificial Sequence Synthetic polynucleotide
gauauugugacauaucucugcacucaucacacaaaagaugcaaugugaguauaggauccacagggagggggcauuuuaacauaucucgcauucaaucauc

SEQ ID NO: 675 - RNA, 100 nt, Artificial Sequence
  Description of Artificial Sequence Synthetic polynucleotide
gauauugugacauaucucugcacucaucacacaaaagaugcaaccuagguauaggauccacagggagggggcauuucaauauaucucaaaucgaaucauc

SEQ ID NO: 676 - RNA, 100 nt, Artificial Sequence
  Description of Artificial Sequence Synthetic polynucleotide
gauauugugacauaucucugcacucaucacacaaaagaugcaaucacaguauaggauccacagggagggggcauucuaauauaucucauauuaaaucauc

SEQ ID NO: 677 - RNA, 100 nt, Artificial Sequence
  Description of Artificial Sequence Synthetic polynucleotide
gauauugugacauaucucugcacucaucacacaaaagaugcaaccuaaguauaggauccacagggagggggcaucuuaauauaucucgguguaaaucauc

SEQ ID NO: 678 - RNA, 100 nt, Artificial Sequence
  Description of Artificial Sequence Synthetic polynucleotide
gauauugugacauaucucugcacucaucacacaaaagaugcaacgaagguauaggauccacagggagggggcauuuuaauauaucuccccuagaaucauc

SEQ ID NO: 679 - RNA, 100 nt, Artificial Sequence
  Description of Artificial Sequence Synthetic polynucleotide
gauauugugacauaucucugcacucaucacacaaaagaugcacuaacccuauaggauccacagggagggggcauuuuaacauaucucaccuagaaucauc

SEQ ID NO: 680 - RNA, 100 nt, Artificial Sequence
  Description of Artificial Sequence Synthetic polynucleotide
gauauugugacauaucucugcacucaucacacaaaagaugcacuggaccuauaggauccacagggagggggcauuucaauauaucucacccgaaaucauc

SEQ ID NO: 681 - RNA, 100 nt, Artificial Sequence
  Description of Artificial Sequence Synthetic polynucleotide
gauauugugacauaucucugcacucaucacacaaaagaugcacucaggcuauaggauccacagggagggggcaucuuaauauaucucgcucgaaaucauc

SEQ ID NO: 682 - RNA, 100 nt, Artificial Sequence
  Description of Artificial Sequence Synthetic polynucleotide
gauauugugacauaucucugcacucaucacacaaaagaugcacuaagacuauaggauccacagggagggggcauuuuaauauaucucaccuucaaucauc

SEQ ID NO: 683 - RNA, 100 nt, Artificial Sequence
  Description of Artificial Sequence Synthetic polynucleotide
gauauugugacauaucucugcacucaucacacaaaagaugcucacgaucuauaggauccacagggagggggcauuuuaacauaucucaaugugaaucauc

SEQ ID NO: 684 - RNA, 100 nt, Artificial Sequence
  Description of Artificial Sequence Synthetic polynucleotide
gauauugugacauaucucugcacucaucacacaaaagaugcccacugucuauaggauccacagggagggggcauuucaauauaucuccacagaaaucauc

SEQ ID NO: 685 - RNA, 100 nt, Artificial Sequence
  Description of Artificial Sequence Synthetic polynucleotide
gauauugugacauaucucugcacucaucacacaaaagaugcuaccugucuauaggauccacagggagggggcauucuaauauaucuccacgagaaucauc

SEQ ID NO: 686 - RNA, 100 nt, Artificial Sequence
  Description of Artificial Sequence Synthetic polynucleotide
gauauugugacauaucucugcacucaucacacaaaagaugcuacuaaucuauaggauccacagggagggggcaucuuaauauaucucgcaugaaaucauc

SEQ ID NO: 687 - RNA, 100 nt, Artificial Sequence
  Description of Artificial Sequence Synthetic polynucleotide
gauauugugacauaucucugcacucaucacacaaaagaugcuucgggucuauaggauccacagggagggggcauuuuaauauaucucacauggaaucauc

SEQ ID NO: 688 - RNA, 100 nt, Artificial Sequence
  Description of Artificial Sequence Synthetic polynucleotide
gauauugugacauaucucugcacucaucacacaaaagauggcuugauucuauaggauccacagggagggggcauuuuaacauaucuccuucagaaucauc

SEQ ID NO: 689 - RNA, 100 nt, Artificial Sequence
  Description of Artificial Sequence Synthetic polynucleotide
gauauugugacauaucucugcacucaucacacaaaagaugaauaaauucuauaggauccacagggagggggcauuucaauauaucucacucgaaaucauc

SEQ ID NO: 690 - RNA, 100 nt, Artificial Sequence
  Description of Artificial Sequence Synthetic polynucleotide
gauauugugacauaucucugcacucaucacacaaaagaugacccaauucuauaggauccacagggagggggcauucuaauauaucucgauucgaaucauc

SEQ ID NO: 691 - RNA, 100 nt, Artificial Sequence
  Description of Artificial Sequence Synthetic polynucleotide
gauauugugacauaucucugcacucaucacacaaaagauggcguuauucuauaggauccacagggagggggcaucuuaauauaucucauuuaaaaucauc

SEQ ID NO: 692 - RNA, 100 nt, Artificial Sequence
  Description of Artificial Sequence Synthetic polynucleotide
gauauugugacauaucucugcacucaucacacaaaagaugaaugaauucuauaggauccacagggagggggcauuuuaauauaucucgcauggaaucauc

SEQ ID NO: 693 - RNA, 100 nt, Artificial Sequence
  Description of Artificial Sequence Synthetic polynucleotide
gauauugugacauaucucugcacucaucacacaaaagauuuuccacuucuauaggauccacagggagggggcauuuuaacauaucuccaccaaaaucauc

SEQ ID NO: 694 - RNA, 100 nt, Artificial Sequence
  Description of Artificial Sequence Synthetic polynucleotide
gauauugugacauaucucugcacucaucacacaaaagaucuuccccuucuauaggauccacagggagggggcauuucaauauaucucacucgaaaucauc

SEQ ID NO: 695 - RNA, 100 nt, Artificial Sequence
  Description of Artificial Sequence Synthetic polynucleotide
gauauugugacauaucucugcacucaucacacaaaagaucccuagcuucuauaggauccacagggagggggcauucuaauauaucucaucgggaaucauc

SEQ ID NO: 696 - RNA, 100 nt, Artificial Sequence
  Description of Artificial Sequence Synthetic polynucleotide
gauauugugacauaucucugcacucaucacacaaaagauuucgugcuucuauaggauccacagggagggggcaucuuaauauaucucacuuucaaucauc

SEQ ID NO: 697 - RNA, 100 nt, Artificial Sequence
  Description of Artificial Sequence Synthetic polynucleotide
gauauugugacauaucucugcacucaucacacaaaagauaggucgcuucuauaggauccacagggagggggcauuuuaauauaucucccuccgaaucauc

SEQ ID NO: 698 - RNA, 100 nt, Artificial Sequence
  Description of Artificial Sequence Synthetic polynucleotide
gauauugugacauaucucugcacucaucacacaaaagacagguuucuucuauaggauccacagggagggggcauuuuaacauaucucgcuuaaaaucauc

SEQ ID NO: 699 - RNA, 100 nt, Artificial Sequence
  Description of Artificial Sequence Synthetic polynucleotide
gauauugugacauaucucugcacucaucacacaaaagagaaacuucuucuauaggauccacagggagggggcauuucaauauaucuccuucgaaaucauc

SEQ ID NO: 700 - RNA, 100 nt, Artificial Sequence
  Description of Artificial Sequence Synthetic polynucleotide
gauauugugacauaucucugcacucaucacacaaaagaaaauuuucuucuauaggauccacagggagggggcauucuaauauaucuccccuggaaucauc

SEQ ID NO: 701 - RNA, 100 nt, Artificial Sequence
  Description of Artificial Sequence Synthetic polynucleotide
gauauugugacauaucucugcacucaucacacaaaagagucuucucuucuauaggauccacagggagggggcaucuuaauauaucuccuuuugaaucauc

SEQ ID NO: 702 - RNA, 100 nt, Artificial Sequence
  Description of Artificial Sequence Synthetic polynucleotide
gauauugugacauaucucugcacucaucacacaaaagaacucuuucuucuauaggauccacagggagggggcauuuuaauauaucucauuccgaaucauc

SEQ ID NO: 703 - RNA, 100 nt, Artificial Sequence
  Description of Artificial Sequence Synthetic polynucleotide
gauauugugacauaucucugcacucaucacacaaaaguaagacaucuucuauaggauccacagggagggggcauuuuaacauaucucccauagaaucauc

SEQ ID NO: 704 - RNA, 100 nt, Artificial Sequence
  Description of Artificial Sequence Synthetic polynucleotide
gauauugugacauaucucugcacucaucacacaaaagcgauucaucuucuauaggauccacagggagggggcauuucaauauaucucgaccccaaucauc

SEQ ID NO: 705 - RNA, 100 nt, Artificial Sequence
  Description of Artificial Sequence Synthetic polynucleotide
gauauugugacauaucucugcacucaucacacaaaagcgagucaucuucuauaggauccacagggagggggcauucuaauauaucucggucgaaaucauc

SEQ ID NO: 706 - RNA, 100 nt, Artificial Sequence
  Description of Artificial Sequence Synthetic polynucleotide
gauauugugacauaucucugcacucaucacacaaaagcauaucaucuucuauaggauccacagggagggggcaucuuaauauaucucgccccaaaucauc

SEQ ID NO: 707 - RNA, 100 nt, Artificial Sequence
  Description of Artificial Sequence Synthetic polynucleotide
gauauugugacauaucucugcacucaucacacaaaagcguuucaucuucuauaggauccacagggagggggcauuuuaauauaucucacugugaaucauc

SEQ ID NO: 708 - RNA, 100 nt, Artificial Sequence
  Description of Artificial Sequence Synthetic polynucleotide
gauauugugacauaucucugcacucaucacacaaaaaucacuaaucuucuauaggauccacagggagggggcauuuuaacauaucucauugggaaucauc

SEQ ID NO: 709 - RNA, 100 nt, Artificial Sequence
  Description of Artificial Sequence Synthetic polynucleotide
gauauugugacauaucucugcacucaucacacaaaaucacuuaaucuucuauaggauccacagggagggggcauuucaauauaucucaaguuaaaucauc

SEQ ID NO: 710 - RNA, 100 nt, Artificial Sequence
  Description of Artificial Sequence Synthetic polynucleotide
gauauugugacauaucucugcacucaucacacaaaaaaacuuaaucuucuauaggauccacagggagggggcauucuaauauaucuccaccagaaucauc

SEQ ID NO: 711 - RNA, 100 nt, Artificial Sequence
  Description of Artificial Sequence Synthetic polynucleotide
gauauugugacauaucucugcacucaucacacaaaaauucucaaucuucuauaggauccacagggagggggcaucuuaauauaucucacagggaaucauc

SEQ ID NO: 712 - RNA, 100 nt, Artificial Sequence
  Description of Artificial Sequence Synthetic polynucleotide
gauauugugacauaucucugcacucaucacacaaaaauaaacaaucuucuauaggauccacagggagggggcauuuuaauauaucuccacgagaaucauc

SEQ ID NO: 713 - RNA, 100 nt, Artificial Sequence
  Description of Artificial Sequence Synthetic polynucleotide
gauauugugacauaucucugcacucaucacacaaauaucagaaaucuucuauaggauccacagggagggggcauuuuaacauaucucgucuaaaaucauc

SEQ ID NO: 714 - RNA, 100 nt, Artificial Sequence
  Description of Artificial Sequence Synthetic polynucleotide
gauauugugacauaucucugcacucaucacacaaaccuggaaaaucuucuauaggauccacagggagggggcauuucaauauaucucauacccaaucauc

SEQ ID NO: 715 - RNA, 100 nt, Artificial Sequence
  Description of Artificial Sequence Synthetic polynucleotide
gauauugugacauaucucugcacucaucacacaaaccucgaaaaucuucuauaggauccacagggagggggcauucuaauauaucucccguaaaaucauc

SEQ ID NO: 716 - RNA, 100 nt, Artificial Sequence
  Description of Artificial Sequence Synthetic polynucleotide
gauauugugacauaucucugcacucaucacacaaaccuacaaaaucuucuauaggauccacagggagggggcauuuuaauauaucucacacccaaucauc

SEQ ID NO: 717 - RNA, 100 nt, Artificial Sequence
  Description of Artificial Sequence Synthetic polynucleotide
gauauugugacauaucucugcacucaucacacaaucagcccaaaucuucuauaggauccacagggagggggcauuuuaacauaucucaccagaaaucauc

SEQ ID NO: 718 - RNA, 100 nt, Artificial Sequence
  Description of Artificial Sequence Synthetic polynucleotide
gauauugugacauaucucugcacucaucacacaacgcauucaaaucuucuauaggauccacagggagggggcauuucaauauaucucccguaaaaucauc

SEQ ID NO: 719 - RNA, 100 nt, Artificial Sequence
  Description of Artificial Sequence Synthetic polynucleotide
gauauugugacauaucucugcacucaucacacaacccacacaaaucuucuauaggauccacagggagggggcauucuaauauaucuccauuucaaucauc

SEQ ID NO: 720 - RNA, 100 nt, Artificial Sequence
  Description of Artificial Sequence Synthetic polynucleotide
gauauugugacauaucucugcacucaucacacaacacacucaaaucuucuauaggauccacagggagggggcaucuuaauauaucucacaccaaaucauc

SEQ ID NO: 721 - RNA, 100 nt, Artificial Sequence
  Description of Artificial Sequence Synthetic polynucleotide
gauauugugacauaucucugcacucaucacacaacccauucaaaucuucuauaggauccacagggagggggcauuuuaauauaucucgacaaaaaucauc

SEQ ID NO: 722 - RNA, 100 nt, Artificial Sequence
  Description of Artificial Sequence Synthetic polynucleotide
gauauugugacauaucucugcacucaucacacaucgcgcgcaaaucuucuauaggauccacagggagggggcauuuuaacauaucuccuccgaaaucauc

SEQ ID NO: 723 - RNA, 100 nt, Artificial Sequence
  Description of Artificial Sequence Synthetic polynucleotide
gauauugugacauaucucugcacucaucacacauguccagcaaaucuucuauaggauccacagggagggggcauuucaauauaucucguucaaaaucauc

SEQ ID NO: 724 - RNA, 100 nt, Artificial Sequence
  Description of Artificial Sequence Synthetic polynucleotide
gauauugugacauaucucugcacucaucacacaccuugcgcaaaucuucuauaggauccacagggagggggcauucuaauauaucucguuuucaaucauc

SEQ ID NO: 725 - RNA, 100 nt, Artificial Sequence
  Description of Artificial Sequence Synthetic polynucleotide
gauauugugacauaucucugcacucaucacacaucgucggcaaaucuucuauaggauccacagggagggggcaucuuaauauaucuccuuccgaaucauc

SEQ ID NO: 726 - RNA, 100 nt, Artificial Sequence
  Description of Artificial Sequence Synthetic polynucleotide
gauauugugacauaucucugcacucaucacacauuaccggcaaaucuucuauaggauccacagggagggggcauuuuaauauaucucacagagaaucauc

SEQ ID NO: 727 - RNA, 100 nt, Artificial Sequence
  Description of Artificial Sequence Synthetic polynucleotide
gauauugugacauaucucugcacucaucacaccucgauugcaaaucuucuauaggauccacagggagggggcauuuuaacauaucucacuucgaaucauc

SEQ ID NO: 728 - RNA, 100 nt, Artificial Sequence
  Description of Artificial Sequence Synthetic polynucleotide
gauauugugacauaucucugcacucaucacaccuccauugcaaaucuucuauaggauccacagggagggggcauuucaauauaucucccacaaaaucauc

SEQ ID NO: 729 - RNA, 100 nt, Artificial Sequence
  Description of Artificial Sequence Synthetic polynucleotide
gauauugugacauaucucugcacucaucacacugcgcuugcaaaucuucuauaggauccacagggagggggcauucuaauauaucucacacggaaucauc

SEQ ID NO: 730 - RNA, 100 nt, Artificial Sequence
  Description of Artificial Sequence Synthetic polynucleotide
gauauugugacauaucucugcacucaucacacuucguuugcaaaucuucuauaggauccacagggagggggcaucuuaauauaucuccauuggaaucauc

SEQ ID NO: 731 - RNA, 100 nt, Artificial Sequence
  Description of Artificial Sequence Synthetic polynucleotide
gauauugugacauaucucugcacucaucacacuuauccugcaaaucuucuauaggauccacagggagggggcauuuuaauauaucucggugagaaucauc

SEQ ID NO: 732 - RNA, 100 nt, Artificial Sequence
  Description of Artificial Sequence Synthetic polynucleotide
gauauugugacauaucucugcacucaucacaacgcuaaugcaaaucuucuauaggauccacagggagggggcauuuuaacauaucuccaccaaaaucauc

SEQ ID NO: 733 - RNA, 100 nt, Artificial Sequence
  Description of Artificial Sequence Synthetic polynucleotide
gauauugugacauaucucugcacucaucacagucugcaugcaaaucuucuauaggauccacagggagggggcauuucaauauaucuccaucgaaaucauc

SEQ ID NO: 734 - RNA, 100 nt, Artificial Sequence
  Description of Artificial Sequence Synthetic polynucleotide
gauauugugacauaucucugcacucaucacaguccaaaugcaaaucuucuauaggauccacagggagggggcauucuaauauaucuccuccccaaucauc

SEQ ID NO: 735 - RNA, 100 nt, Artificial Sequence
  Description of Artificial Sequence Synthetic polynucleotide
gauauugugacauaucucugcacucaucacaauuugaaugcaaaucuucuauaggauccacagggagggggcaucuuaauauaucucgacaggaaucauc

SEQ ID NO: 736 - RNA, 100 nt, Artificial Sequence
  Description of Artificial Sequence Synthetic polynucleotide
gauauugugacauaucucugcacucaucacaauguacaugcaaaucuucuauaggauccacagggagggggcauuuuaauauaucucgcggggaaucauc

SEQ ID NO: 737 - RNA, 100 nt, Artificial Sequence
  Description of Artificial Sequence Synthetic polynucleotide
gauauugugacauaucucugcacucaucacugccgugaugcaaaucuucuauaggauccacagggagggggcauuuuaacauaucucgguuucaaucauc

SEQ ID NO: 738 - RNA, 100 nt, Artificial Sequence
  Description of Artificial Sequence Synthetic polynucleotide
gauauugugacauaucucugcacucaucacuugguugaugcaaaucuucuauaggauccacagggagggggcauuucaauauaucucguuuaaaaucauc

SEQ ID NO: 739 - RNA, 100 nt, Artificial Sequence
  Description of Artificial Sequence Synthetic polynucleotide
gauauugugacauaucucugcacucaucaccccuuugaugcaaaucuucuauaggauccacagggagggggcauucuaauauaucucaaguagaaucauc

SEQ ID NO: 740 - RNA, 100 nt, Artificial Sequence
  Description of Artificial Sequence Synthetic polynucleotide
gauauugugacauaucucugcacucaucaccuuuucgaugcaaaucuucuauaggauccacagggagggggcaucuuaauauaucucgauucgaaucauc

SEQ ID NO: 741 - RNA, 100 nt, Artificial Sequence
  Description of Artificial Sequence Synthetic polynucleotide
gauauugugacauaucucugcacucaucacugugcugaugcaaaucuucuauaggauccacagggagggggcauuuuaauauaucucccauggaaucauc

SEQ ID NO: 742 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaucuggcucauaaucauc

SEQ ID NO: 743 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaucugccgcauaaucauc

SEQ ID NO: 744 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauaucuaacuaauaaucauc

SEQ ID NO: 745 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaucuaacacauaaucauc

SEQ ID NO: 746 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauaucuggcccguaaucauc

SEQ ID NO: 747 - RNA, 100 nt, Artificial Sequence
  Description of Artificial Sequence Synthetic polynucleotide
gauauugugacauaucucugcacucaucacacaaaagaugcaaaucuucuauagaaaagucagggagggggcauuucaauauaucuaaauuguaaucauc

SEQ ID NO: 748 - RNA, 100 nt, Artificial Sequence
  Description of Artificial Sequence Synthetic polynucleotide
gauauugugacauaucucugcacucaucacacaaaagaugcaaaucuucuauagcgcaaucagggagggggcaucuuaauauaucuacgucguaaucauc

SEQ ID NO: 749 - RNA, 100 nt, Artificial Sequence
  Description of Artificial Sequence Synthetic polynucleotide
gauauugugacauaucucugcacucaucacacaaaagaugcaaaucuucuauagcucagucagggagggggcauuuuaauauaucuggucaauaaucauc

SEQ ID NO: 750 - RNA, 100 nt, Artificial Sequence
  Description of Artificial Sequence Synthetic polynucleotide
gauauugugacauaucucugcacucaucacacaaaagaugcaaaucuucuauaacugagacagggagggggcauuuuaacauaucuaaaucguaaucauc

SEQ ID NO: 751 - RNA, 100 nt, Artificial Sequence
  Description of Artificial Sequence Synthetic polynucleotide
gauauugugacauaucucugcacucaucacacaaaagaugcaaaucuucuauaucucaaacagggagggggcauuucaauauaucuacaauauaaucauc

SEQ ID NO: 752 - RNA, 100 nt, Artificial Sequence
  Description of Artificial Sequence Synthetic polynucleotide
gauauugugacauaucucugcacucaucacacaaaagaugcaaaucuucuauauuaagaacagggagggggcaucuuaauauaucuacgcuauaaucauc

SEQ ID NO: 753 - RNA, 100 nt, Artificial Sequence
  Description of Artificial Sequence Synthetic polynucleotide
gauauugugacauaucucugcacucaucacacaaaagaugcaaaucuucuauaaccuaaacagggagggggcauuuuaauauaucuauuccauaaucauc

SEQ ID NO: 754 - RNA, 100 nt, Artificial Sequence
  Description of Artificial Sequence Synthetic polynucleotide
gauauugugacauaucucugcacucaucacacaaaagaugcaaaucuucuauuauugacacagggagggggcauuuuaacauaucuaaccuguaaucauc

SEQ ID NO: 755 - RNA, 100 nt, Artificial Sequence
  Description of Artificial Sequence Synthetic polynucleotide
gauauugugacauaucucugcacucaucacacaaaagaugcaaaucuucuauuacuagcacagggagggggcauuucaauauaucuggucgauaaucauc

SEQ ID NO: 756 - RNA, 100 nt, Artificial Sequence
  Description of Artificial Sequence Synthetic polynucleotide
gauauugugacauaucucugcacucaucacacaaaagaugcaaaucuucuaucuagcacacagggagggggcauucuaauauaucugcuugauaaucauc

SEQ ID NO: 757 - RNA, 100 nt, Artificial Sequence
  Description of Artificial Sequence Synthetic polynucleotide
gauauugugacauaucucugcacucaucacacaaaagaugcaaaucuucuauuucccgcacagggagggggcaucuuaauauaucuaagcaauaaucauc

SEQ ID NO: 758 - RNA, 100 nt, Artificial Sequence
  Description of Artificial Sequence Synthetic polynucleotide
gauauugugacauaucucugcacucaucacacaaaagaugcaaaucuucuaucaccggcacagggagggggcauuuuaauauaucuaauucguaaucauc

SEQ ID NO: 759 - RNA, 100 nt, Artificial Sequence
  Description of Artificial Sequence Synthetic polynucleotide
gauauugugacauaucucugcacucaucacacaaaagaugcaaaucuucuagccuacccacagggagggggcauuuuaacauaucuggcucauaaucauc

SEQ ID NO: 760 - RNA, 100 nt, Artificial Sequence
  Description of Artificial Sequence Synthetic polynucleotide
gauauugugacauaucucugcacucaucacacaaaagaugcaaaucuucuagcucgcccacagggagggggcauuucaauauaucugacccauaaucauc

SEQ ID NO: 761 - RNA, 100 nt, Artificial Sequence
  Description of Artificial Sequence Synthetic polynucleotide
gauauugugacauaucucugcacucaucacacaaaagaugcaaaucuucuaacaacgccacagggagggggcauucuaauauaucuggcuuauaaucauc

SEQ ID NO: 762 - RNA, 100 nt, Artificial Sequence
  Description of Artificial Sequence Synthetic polynucleotide
gauauugugacauaucucugcacucaucacacaaaagaugcaaaucuucuaaucagcccacagggagggggcaucuuaauauaucugaaguguaaucauc

SEQ ID NO: 763 - RNA, 100 nt, Artificial Sequence
  Description of Artificial Sequence Synthetic polynucleotide
gauauugugacauaucucugcacucaucacacaaaagaugcaaaucuucuagccaccccacagggagggggcauuuuaauauaucuaucacguaaucauc

SEQ ID NO: 764 - RNA, 100 nt, Artificial Sequence
  Description of Artificial Sequence Synthetic polynucleotide
gauauugugacauaucucugcacucaucacacaaaagaugcaaaucuucucucgucuccacagggagggggcauuuuaacauaucugcuccguaaucauc

SEQ ID NO: 765 - RNA, 100 nt, Artificial Sequence
  Description of Artificial Sequence Synthetic polynucleotide
gauauugugacauaucucugcacucaucacacaaaagaugcaaaucuucuccaucuuccacagggagggggcauuucaauauaucugguccauaaucauc

SEQ ID NO: 766 - RNA, 100 nt, Artificial Sequence
  Description of Artificial Sequence Synthetic polynucleotide
gauauugugacauaucucugcacucaucacacaaaagaugcaaaucuucucacaauuccacagggagggggcauucuaauauaucuggcguguaaucauc

SEQ ID NO: 767 - RNA, 100 nt, Artificial Sequence
  Description of Artificial Sequence Synthetic polynucleotide
gauauugugacauaucucugcacucaucacacaaaagaugcaaaucuucuuaacguuccacagggagggggcaucuuaauauaucuacuugauaaucauc

SEQ ID NO: 768 - RNA, 100 nt, Artificial Sequence
  Description of Artificial Sequence Synthetic polynucleotide
gauauugugacauaucucugcacucaucacacaaaagaugcaaaucuucuuguauuuccacagggagggggcauuuuaauauaucuaagccauaaucauc

SEQ ID NO: 769 - RNA, 100 nt, Artificial Sequence
  Description of Artificial Sequence Synthetic polynucleotide
gauauugugacauaucucugcacucaucacacaaaagaugcaaaucuucgacuccauccacagggagggggcauuuuaacauaucuaaaauauaaucauc

SEQ ID NO: 770 - RNA, 100 nt, Artificial Sequence
  Description of Artificial Sequence Synthetic polynucleotide
gauauugugacauaucucugcacucaucacacaaaagaugcaaaucuucgaagaaauccacagggagggggcauuucaauauaucugccgcauaaucauc

SEQ ID NO: 771 - RNA, 100 nt, Artificial Sequence
  Description of Artificial Sequence Synthetic polynucleotide
gauauugugacauaucucugcacucaucacacaaaagaugcaaaucuucaucaccauccacagggagggggcauucuaauauaucuaaauuauaaucauc

SEQ ID NO: 772 - RNA, 100 nt, Artificial Sequence
  Description of Artificial Sequence Synthetic polynucleotide
gauauugugacauaucucugcacucaucacacaaaagaugcaaaucuucacguuuauccacagggagggggcaucuuaauauaucuacuauguaaucauc

SEQ ID NO: 773 - RNA, 100 nt, Artificial Sequence
  Description of Artificial Sequence Synthetic polynucleotide
gauauugugacauaucucugcacucaucacacaaaagaugcaaaucuuccccuuuauccacagggagggggcauuuuaauauaucuauuuuguaaucauc

SEQ ID NO: 774 - RNA, 100 nt, Artificial Sequence
  Description of Artificial Sequence Synthetic polynucleotide
gauauugugacauaucucugcacucaucacacaaaagaugcaaaucuuggaauagauccacagggagggggcauuuuaacauaucuacuugauaaucauc

SEQ ID NO: 775 - RNA, 100 nt, Artificial Sequence
  Description of Artificial Sequence Synthetic polynucleotide
gauauugugacauaucucugcacucaucacacaaaagaugcaaaucuugaacgcgauccacagggagggggcauuucaauauaucugcacuguaaucauc

SEQ ID NO: 776 - RNA, 100 nt, Artificial Sequence
  Description of Artificial Sequence Synthetic polynucleotide
gauauugugacauaucucugcacucaucacacaaaagaugcaaaucuugauaucgauccacagggagggggcauucuaauauaucuaguuaauaaucauc

SEQ ID NO: 777 - RNA, 100 nt, Artificial Sequence
  Description of Artificial Sequence Synthetic polynucleotide
gauauugugacauaucucugcacucaucacacaaaagaugcaaaucuuauaccugauccacagggagggggcaucuuaauauaucuauaguguaaucauc

SEQ ID NO: 778 - RNA, 100 nt, Artificial Sequence
  Description of Artificial Sequence Synthetic polynucleotide
gauauugugacauaucucugcacucaucacacaaaagaugcaaaucuuaggcucgauccacagggagggggcauuuuaauauaucuaacacauaaucauc

SEQ ID NO: 779 - RNA, 100 nt, Artificial Sequence
  Description of Artificial Sequence Synthetic polynucleotide
gauauugugacauaucucugcacucaucacacaaaagaugcaaaucuaggcguggauccacagggagggggcauuuuaacauaucuaacucguaaucauc

SEQ ID NO: 780 - RNA, 100 nt, Artificial Sequence
  Description of Artificial Sequence Synthetic polynucleotide
gauauugugacauaucucugcacucaucacacaaaagaugcaaaucuguguuuggauccacagggagggggcauuucaauauaucuaguuaauaaucauc

SEQ ID NO: 781 - RNA, 100 nt, Artificial Sequence
  Description of Artificial Sequence Synthetic polynucleotide
gauauugugacauaucucugcacucaucacacaaaagaugcaaaucucaucauggauccacagggagggggcauucuaauauaucuaccucauaaucauc

SEQ ID NO: 782 - RNA, 100 nt, Artificial Sequence
  Description of Artificial Sequence Synthetic polynucleotide
gauauugugacauaucucugcacucaucacacaaaagaugcaaaucuagcaguggauccacagggagggggcaucuuaauauaucuggccuauaaucauc

SEQ ID NO: 783 - RNA, 100 nt, Artificial Sequence
  Description of Artificial Sequence Synthetic polynucleotide
gauauugugacauaucucugcacucaucacacaaaagaugcaaaucuccacucggauccacagggagggggcauuuuaauauaucugaccuauaaucauc

SEQ ID NO: 784 - RNA, 100 nt, Artificial Sequence
  Description of Artificial Sequence Synthetic polynucleotide
gauauugugacauaucucugcacucaucacacaaaagaugcaaauccaguucaggauccacagggagggggcauuuuaacauaucuaccuuguaaucauc

SEQ ID NO: 785 - RNA, 100 nt, Artificial Sequence
  Description of Artificial Sequence Synthetic polynucleotide
gauauugugacauaucucugcacucaucacacaaaagaugcaaaucggaucgaggauccacagggagggggcauuucaauauaucuaguuuguaaucauc

SEQ ID NO: 786 - RNA, 100 nt, Artificial Sequence
  Description of Artificial Sequence Synthetic polynucleotide
gauauugugacauaucucugcacucaucacacaaaagaugcaaaucgugaugaggauccacagggagggggcauucuaauauaucuggcgcguaaucauc

SEQ ID NO: 787 - RNA, 100 nt, Artificial Sequence
  Description of Artificial Sequence Synthetic polynucleotide
gauauugugacauaucucugcacucaucacacaaaagaugcaaaucgaccucaggauccacagggagggggcaucuuaauauaucugcgcgauaaucauc

SEQ ID NO: 788 - RNA, 100 nt, Artificial Sequence
  Description of Artificial Sequence Synthetic polynucleotide
gauauugugacauaucucugcacucaucacacaaaagaugcaaauccgagcaaggauccacagggagggggcauuuuaauauaucugcaguguaaucauc

SEQ ID NO: 789 - RNA, 100 nt, Artificial Sequence
  Description of Artificial Sequence Synthetic polynucleotide
gauauugugacauaucucugcacucaucacacaaaagaugcaaauaugaucuaggauccacagggagggggcauuuuaacauaucuaguauauaaucauc

SEQ ID NO: 790 - RNA, 100 nt, Artificial Sequence
  Description of Artificial Sequence Synthetic polynucleotide
gauauugugacauaucucugcacucaucacacaaaagaugcaaauggugccuaggauccacagggagggggcauuucaauauaucugauguauaaucauc

SEQ ID NO: 791 - RNA, 100 nt, Artificial Sequence
  Description of Artificial Sequence Synthetic polynucleotide
gauauugugacauaucucugcacucaucacacaaaagaugcaaauacagguuaggauccacagggagggggcauucuaauauaucuaacggauaaucauc

SEQ ID NO: 792 - RNA, 100 nt, Artificial Sequence
  Description of Artificial Sequence Synthetic polynucleotide
gauauugugacauaucucugcacucaucacacaaaagaugcaaaugacguuuaggauccacagggagggggcaucuuaauauaucuacguuguaaucauc

SEQ ID NO: 793 - RNA, 100 nt, Artificial Sequence
  Description of Artificial Sequence Synthetic polynucleotide
gauauugugacauaucucugcacucaucacacaaaagaugcaaauaucaauuaggauccacagggagggggcauuuuaauauaucugaccuauaaucauc

SEQ ID NO: 794 - RNA, 100 nt, Artificial Sequence
  Description of Artificial Sequence Synthetic polynucleotide
gauauugugacauaucucugcacucaucacacaaaagaugcaaaagaacgauaggauccacagggagggggcauuuuaacauaucuaccauguaaucauc

SEQ ID NO: 795 - RNA, 100 nt, Artificial Sequence
  Description of Artificial Sequence Synthetic polynucleotide
gauauugugacauaucucugcacucaucacacaaaagaugcaaaggguggauaggauccacagggagggggcauuucaauauaucuagcucauaaucauc

SEQ ID NO: 796 - RNA, 100 nt, Artificial Sequence
  Description of Artificial Sequence Synthetic polynucleotide
gauauugugacauaucucugcacucaucacacaaaagaugcaaagagccgauaggauccacagggagggggcauucuaauauaucuggccuauaaucauc

SEQ ID NO: 797 - RNA, 100 nt, Artificial Sequence
  Description of Artificial Sequence Synthetic polynucleotide
gauauugugacauaucucugcacucaucacacaaaagaugcaaaauggggauaggauccacagggagggggcaucuuaauauaucuggcuuguaaucauc

SEQ ID NO: 798 - RNA, 100 nt, Artificial Sequence
  Description of Artificial Sequence Synthetic polynucleotide
gauauugugacauaucucugcacucaucacacaaaagaugcaaaauacacauaggauccacagggagggggcauuuuaauauaucugacucauaaucauc

SEQ ID NO: 799 - RNA, 100 nt, Artificial Sequence
  Description of Artificial Sequence Synthetic polynucleotide
gauauugugacauaucucugcacucaucacacaaaagaugcaacggaaauauaggauccacagggagggggcauuuuaacauaucuaauuuauaaucauc

SEQ ID NO: 800 - RNA, 100 nt, Artificial Sequence
  Description of Artificial Sequence Synthetic polynucleotide
gauauugugacauaucucugcacucaucacacaaaagaugcaacagguauauaggauccacagggagggggcauuucaauauaucuggcggauaaucauc

SEQ ID NO: 801 - RNA, 100 nt, Artificial Sequence
  Description of Artificial Sequence Synthetic polynucleotide
gauauugugacauaucucugcacucaucacacaaaagaugcaaccgacguauaggauccacagggagggggcauucuaauauaucuaacucguaaucauc

SEQ ID NO: 802 - RNA, 100 nt, Artificial Sequence
  Description of Artificial Sequence Synthetic polynucleotide
gauauugugacauaucucugcacucaucacacaaaagaugcaauugccauauaggauccacagggagggggcaucuuaauauaucuauuuuguaaucauc

SEQ ID NO: 803 - RNA, 100 nt, Artificial Sequence
  Description of Artificial Sequence Synthetic polynucleotide
gauauugugacauaucucugcacucaucacacaaaagaugcaacguacauauaggauccacagggagggggcauuuuaauauaucugauugauaaucauc

SEQ ID NO: 804 - RNA, 100 nt, Artificial Sequence
  Description of Artificial Sequence Synthetic polynucleotide
gauauugugacauaucucugcacucaucacacaaaagaugcauaagcacuauaggauccacagggagggggcauuuuaacauaucugaaauauaaucauc

SEQ ID NO: 805 - RNA, 100 nt, Artificial Sequence
  Description of Artificial Sequence Synthetic polynucleotide
gauauugugacauaucucugcacucaucacacaaaagaugcacuaagccuauaggauccacagggagggggcauuucaauauaucugcaguguaaucauc

SEQ ID NO: 806 - RNA, 100 nt, Artificial Sequence
  Description of Artificial Sequence Synthetic polynucleotide
gauauugugacauaucucugcacucaucacacaaaagaugcaccugcccuauaggauccacagggagggggcauucuaauauaucuggcuaauaaucauc

SEQ ID NO: 807 - RNA, 100 nt, Artificial Sequence
  Description of Artificial Sequence Synthetic polynucleotide
gauauugugacauaucucugcacucaucacacaaaagaugcauacgcgcuauaggauccacagggagggggcaucuuaauauaucuaccccguaaucauc

SEQ ID NO: 808 - RNA, 100 nt, Artificial Sequence
  Description of Artificial Sequence Synthetic polynucleotide
gauauugugacauaucucugcacucaucacacaaaagaugcauccacacuauaggauccacagggagggggcauuuuaauauaucuaacgcauaaucauc

SEQ ID NO: 809 - RNA, 100 nt, Artificial Sequence
  Description of Artificial Sequence Synthetic polynucleotide
gauauugugacauaucucugcacucaucacacaaaagaugcuauaaaucuauaggauccacagggagggggcauuuuaacauaucugcccgauaaucauc

SEQ ID NO: 810 - RNA, 100 nt, Artificial Sequence
  Description of Artificial Sequence Synthetic polynucleotide
gauauugugacauaucucugcacucaucacacaaaagaugcccuauaucuauaggauccacagggagggggcauuucaauauaucuaguuuauaaucauc

SEQ ID NO: 811 - RNA, 100 nt, Artificial Sequence
  Description of Artificial Sequence Synthetic polynucleotide
gauauugugacauaucucugcacucaucacacaaaagaugccccgacucuauaggauccacagggagggggcauucuaauauaucuagccuauaaucauc

SEQ ID NO: 812 - RNA, 100 nt, Artificial Sequence
  Description of Artificial Sequence Synthetic polynucleotide
gauauugugacauaucucugcacucaucacacaaaagaugccaugacucuauaggauccacagggagggggcaucuuaauauaucuaaccuguaaucauc

SEQ ID NO: 813 - RNA, 100 nt, Artificial Sequence
  Description of Artificial Sequence Synthetic polynucleotide
gauauugugacauaucucugcacucaucacacaaaagaugccuuuaaucuauaggauccacagggagggggcauuuuaauauaucuaacgcguaaucauc

SEQ ID NO: 814 - RNA, 100 nt, Artificial Sequence
  Description of Artificial Sequence Synthetic polynucleotide
gauauugugacauaucucugcacucaucacacaaaagaugagcgcauucuauaggauccacagggagggggcauuuuaacauaucugcgcuguaaucauc

SEQ ID NO: 815 - RNA, 100 nt, Artificial Sequence
  Description of Artificial Sequence Synthetic polynucleotide
gauauugugacauaucucugcacucaucacacaaaagauggccugguucuauaggauccacagggagggggcauuucaauauaucugccccguaaucauc

SEQ ID NO: 816 - RNA, 100 nt, Artificial Sequence
  Description of Artificial Sequence Synthetic polynucleotide
gauauugugacauaucucugcacucaucacacaaaagaugaauaaguucuauaggauccacagggagggggcauucuaauauaucuagauuguaaucauc

SEQ ID NO: 817 - RNA, 100 nt, Artificial Sequence
  Description of Artificial Sequence Synthetic polynucleotide
gauauugugacauaucucugcacucaucacacaaaagaugacucgauucuauaggauccacagggagggggcaucuuaauauaucugacucguaaucauc

SEQ ID NO: 818 - RNA, 100 nt, Artificial Sequence
  Description of Artificial Sequence Synthetic polynucleotide
gauauugugacauaucucugcacucaucacacaaaagauggucugguucuauaggauccacagggagggggcauuuuaauauaucuggucuguaaucauc

SEQ ID NO: 819 - RNA, 100 nt, Artificial Sequence
  Description of Artificial Sequence Synthetic polynucleotide
gauauugugacauaucucugcacucaucacacaaaagauaggucgcuucuauaggauccacagggagggggcauuuuaacauaucugacauguaaucauc

SEQ ID NO: 820 - RNA, 100 nt, Artificial Sequence
  Description of Artificial Sequence Synthetic polynucleotide
gauauugugacauaucucugcacucaucacacaaaagauaacugccuucuauaggauccacagggagggggcauuucaauauaucuaauccguaaucauc

SEQ ID NO: 821 - RNA, 100 nt, Artificial Sequence
  Description of Artificial Sequence Synthetic polynucleotide
gauauugugacauaucucugcacucaucacacaaaagauauucuacuucuauaggauccacagggagggggcauucuaauauaucuaccucguaaucauc

SEQ ID NO: 822 - RNA, 100 nt, Artificial Sequence
  Description of Artificial Sequence Synthetic polynucleotide
gauauugugacauaucucugcacucaucacacaaaagauuaugggcuucuauaggauccacagggagggggcauuuuaauauaucugccccguaaucauc

SEQ ID NO: 823 - RNA, 100 nt, Artificial Sequence
  Description of Artificial Sequence Synthetic polynucleotide
gauauugugacauaucucugcacucaucacacaaaagaaaguguucuucuauaggauccacagggagggggcauuuuaacauaucuauuccguaaucauc

SEQ ID NO: 824 - RNA, 100 nt, Artificial Sequence
  Description of Artificial Sequence Synthetic polynucleotide
gauauugugacauaucucugcacucaucacacaaaagaaaauguucuucuauaggauccacagggagggggcauuucaauauaucugguugauaaucauc

SEQ ID NO: 825 - RNA, 100 nt, Artificial Sequence
  Description of Artificial Sequence Synthetic polynucleotide
gauauugugacauaucucugcacucaucacacaaaagaccucuuucuucuauaggauccacagggagggggcauucuaauauaucuaguguguaaucauc

SEQ ID NO: 826 - RNA, 100 nt, Artificial Sequence
  Description of Artificial Sequence Synthetic polynucleotide
gauauugugacauaucucugcacucaucacacaaaagaaaggucucuucuauaggauccacagggagggggcaucuuaauauaucuagacgauaaucauc

SEQ ID NO: 827 - RNA, 100 nt, Artificial Sequence
  Description of Artificial Sequence Synthetic polynucleotide
gauauugugacauaucucugcacucaucacacaaaagagaauguucuucuauaggauccacagggagggggcauuuuaauauaucuggauuguaaucauc

SEQ ID NO: 828 - RNA, 100 nt, Artificial Sequence
  Description of Artificial Sequence Synthetic polynucleotide
gauauugugacauaucucugcacucaucacacaaaagcuauacaucuucuauaggauccacagggagggggcauuuuaacauaucugauucauaaucauc

SEQ ID NO: 829 - RNA, 100 nt, Artificial Sequence
  Description of Artificial Sequence Synthetic polynucleotide
gauauugugacauaucucugcacucaucacacaaaagccuuuuaucuucuauaggauccacagggagggggcauuucaauauaucuagccuauaaucauc

SEQ ID NO: 830 - RNA, 100 nt, Artificial Sequence
  Description of Artificial Sequence Synthetic polynucleotide
gauauugugacauaucucugcacucaucacacaaaagcucauuaucuucuauaggauccacagggagggggcauucuaauauaucuaguucguaaucauc

SEQ ID NO: 831 - RNA, 100 nt, Artificial Sequence
  Description of Artificial Sequence Synthetic polynucleotide
gauauugugacauaucucugcacucaucacacaaaaguaaaccaucuucuauaggauccacagggagggggcaucuuaauauaucuacuauguaaucauc

SEQ ID NO: 832 - RNA, 100 nt, Artificial Sequence
  Description of Artificial Sequence Synthetic polynucleotide
gauauugugacauaucucugcacucaucacacaaaagcgaccuaucuucuauaggauccacagggagggggcauuuuaauauaucuagcccguaaucauc

SEQ ID NO: 833 - RNA, 100 nt, Artificial Sequence
  Description of Artificial Sequence Synthetic polynucleotide
gauauugugacauaucucugcacucaucacacaaaauuucauaaucuucuauaggauccacagggagggggcauuuuaacauaucugcucuguaaucauc

SEQ ID NO: 834 - RNA, 100 nt, Artificial Sequence
  Description of Artificial Sequence Synthetic polynucleotide
gauauugugacauaucucugcacucaucacacaaaacaauauaaucuucuauaggauccacagggagggggcauuucaauauaucuagcauauaaucauc

SEQ ID NO: 835 - RNA, 100 nt, Artificial Sequence
  Description of Artificial Sequence Synthetic polynucleotide
gauauugugacauaucucugcacucaucacacaaaaacuucuaaucuucuauaggauccacagggagggggcauucuaauauaucuacaccauaaucauc

SEQ ID NO: 836 - RNA, 100 nt, Artificial Sequence
  Description of Artificial Sequence Synthetic polynucleotide
gauauugugacauaucucugcacucaucacacaaaaacuaacaaucuucuauaggauccacagggagggggcaucuuaauauaucuauaagauaaucauc

SEQ ID NO: 837 - RNA, 100 nt, Artificial Sequence
  Description of Artificial Sequence Synthetic polynucleotide
gauauugugacauaucucugcacucaucacacaaaauucaacaaucuucuauaggauccacagggagggggcauuuuaauauaucuaccuuguaaucauc

SEQ ID NO: 838 - RNA, 100 nt, Artificial Sequence
  Description of Artificial Sequence Synthetic polynucleotide
gauauugugacauaucucugcacucaucacacaaacucacaaaaucuucuauaggauccacagggagggggcauuuuaacauaucugauccauaaucauc

SEQ ID NO: 839 - RNA, 100 nt, Artificial Sequence
  Description of Artificial Sequence Synthetic polynucleotide
gauauugugacauaucucugcacucaucacacaaauaaguaaaaucuucuauaggauccacagggagggggcauuucaauauaucugacgcauaaucauc

SEQ ID NO: 840 - RNA, 100 nt, Artificial Sequence
  Description of Artificial Sequence Synthetic polynucleotide
gauauugugacauaucucugcacucaucacacaaacuucagaaaucuucuauaggauccacagggagggggcauucuaauauaucuggauuguaaucauc

SEQ ID NO: 841 - RNA, 100 nt, Artificial Sequence
  Description of Artificial Sequence Synthetic polynucleotide
gauauugugacauaucucugcacucaucacacaaaucaaagaaaucuucuauaggauccacagggagggggcaucuuaauauaucuacgugauaaucauc

SEQ ID NO: 842 - RNA, 100 nt, Artificial Sequence
  Description of Artificial Sequence Synthetic polynucleotide
gauauugugacauaucucugcacucaucacacaaaucacugaaaucuucuauaggauccacagggagggggcauuuuaauauaucuaccgcauaaucauc

SEQ ID NO: 843 - RNA, 100 nt, Artificial Sequence
  Description of Artificial Sequence Synthetic polynucleotide
gauauugugacauaucucugcacucaucacacaaccuccacaaaucuucuauaggauccacagggagggggcauuuuaacauaucugcccaauaaucauc

SEQ ID NO: 844 - RNA, 100 nt, Artificial Sequence
  Description of Artificial Sequence Synthetic polynucleotide
gauauugugacauaucucugcacucaucacacaacgucaucaaaucuucuauaggauccacagggagggggcauuucaauauaucuagauuguaaucauc

SEQ ID NO: 845 - RNA, 100 nt, Artificial Sequence
  Description of Artificial Sequence Synthetic polynucleotide
gauauugugacauaucucugcacucaucacacaaucuaaacaaaucuucuauaggauccacagggagggggcauucuaauauaucuaguucguaaucauc

SEQ ID NO: 846 - RNA, 100 nt, Artificial Sequence
  Description of Artificial Sequence Synthetic polynucleotide
gauauugugacauaucucugcacucaucacacauccuaggcaaaucuucuauaggauccacagggagggggcauuuuaacauaucugaaagauaaucauc

SEQ ID NO: 847 - RNA, 100 nt, Artificial Sequence
  Description of Artificial Sequence Synthetic polynucleotide
gauauugugacauaucucugcacucaucacacaucaugcgcaaaucuucuauaggauccacagggagggggcauuucaauauaucuagcguguaaucauc

SEQ ID NO: 848 - RNA, 100 nt, Artificial Sequence
  Description of Artificial Sequence Synthetic polynucleotide
gauauugugacauaucucugcacucaucacacacgcccagcaaaucuucuauaggauccacagggagggggcauucuaauauaucuaacucguaaucauc

SEQ ID NO: 849 - RNA, 100 nt, Artificial Sequence
  Description of Artificial Sequence Synthetic polynucleotide
gauauugugacauaucucugcacucaucacacacuaucggcaaaucuucuauaggauccacagggagggggcaucuuaauauaucugcuccauaaucauc

SEQ ID NO: 850 - RNA, 100 nt, Artificial Sequence
  Description of Artificial Sequence Synthetic polynucleotide
gauauugugacauaucucugcacucaucacacaccguucgcaaaucuucuauaggauccacagggagggggcauuuuaauauaucugacucguaaucauc

SEQ ID NO: 851 - RNA, 100 nt, Artificial Sequence
  Description of Artificial Sequence Synthetic polynucleotide
gauauugugacauaucucugcacucaucacacccgcucugcaaaucuucuauaggauccacagggagggggcauuuuaacauaucuacgucguaaucauc

SEQ ID NO: 852 - RNA, 100 nt, Artificial Sequence
  Description of Artificial Sequence Synthetic polynucleotide
gauauugugacauaucucugcacucaucacacugcguuugcaaaucuucuauaggauccacagggagggggcauuucaauauaucugguuuguaaucauc

SEQ ID NO: 853 - RNA, 100 nt, Artificial Sequence
  Description of Artificial Sequence Synthetic polynucleotide
gauauugugacauaucucugcacucaucacacuuacccugcaaaucuucuauaggauccacagggagggggcauucuaauauaucugaaguguaaucauc

SEQ ID NO: 854 - RNA, 100 nt, Artificial Sequence
  Description of Artificial Sequence Synthetic polynucleotide
gauauugugacauaucucugcacucaucacaccuauacugcaaaucuucuauaggauccacagggagggggcaucuuaauauaucugacuaauaaucauc

SEQ ID NO: 855 - RNA, 100 nt, Artificial Sequence
  Description of Artificial Sequence Synthetic polynucleotide
gauauugugacauaucucugcacucaucacaccaucucugcaaaucuucuauaggauccacagggagggggcauuuuaauauaucuagucuguaaucauc

SEQ ID NO: 856 - RNA, 100 nt, Artificial Sequence
  Description of Artificial Sequence Synthetic polynucleotide
gauauugugacauaucucugcacucaucacaagcucaaugcaaaucuucuauaggauccacagggagggggcauuuuaacauaucugcaguauaaucauc

SEQ ID NO: 857 - RNA, 100 nt, Artificial Sequence
  Description of Artificial Sequence Synthetic polynucleotide
gauauugugacauaucucugcacucaucacagcccaaaugcaaaucuucuauaggauccacagggagggggcauuucaauauaucugcaucguaaucauc

SEQ ID NO: 858 - RNA, 100 nt, Artificial Sequence
  Description of Artificial Sequence Synthetic polynucleotide
gauauugugacauaucucugcacucaucacagucauaaugcaaaucuucuauaggauccacagggagggggcauucuaauauaucugauuuguaaucauc

SEQ ID NO: 859 - RNA, 100 nt, Artificial Sequence
  Description of Artificial Sequence Synthetic polynucleotide
gauauugugacauaucucugcacucaucacaagugcuaugcaaaucuucuauaggauccacagggagggggcaucuuaauauaucugaucuauaaucauc

SEQ ID NO: 860 - RNA, 100 nt, Artificial Sequence
  Description of Artificial Sequence Synthetic polynucleotide
gauauugugacauaucucugcacucaucacaacgcuaaugcaaaucuucuauaggauccacagggagggggcauuuuaauauaucugguuaauaaucauc

SEQ ID NO: 861 - RNA, 100 nt, Artificial Sequence
  Description of Artificial Sequence Synthetic polynucleotide
gauauugugacauaucucugcacucaucaccuggcugaugcaaaucuucuauaggauccacagggagggggcauuuuaacauaucuagcuuguaaucauc

SEQ ID NO: 862 - RNA, 100 nt, Artificial Sequence
  Description of Artificial Sequence Synthetic polynucleotide
gauauugugacauaucucugcacucaucaccuuugcgaugcaaaucuucuauaggauccacagggagggggcauuucaauauaucugguagauaaucauc

SEQ ID NO: 863 - RNA, 100 nt, Artificial Sequence
  Description of Artificial Sequence Synthetic polynucleotide
gauauugugacauaucucugcacucaucaccccgccgaugcaaaucuucuauaggauccacagggagggggcauucuaauauaucugacuaauaaucauc

SEQ ID NO: 864 - RNA, 100 nt, Artificial Sequence
  Description of Artificial Sequence Synthetic polynucleotide
gauauugugacauaucucugcacucaucacuggugcgaugcaaaucuucuauaggauccacagggagggggcaucuuaauauaucugcgcuguaaucauc

SEQ ID NO: 865 - RNA, 100 nt, Artificial Sequence
  Description of Artificial Sequence Synthetic polynucleotide
gauauugugacauaucucugcacucaucacuaauucgaugcaaaucuucuauaggauccacagggagggggcauuuuaauauaucugacaaauaaucauc

SEQ ID NO: 866 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaucauaaaucuaaucauc

SEQ ID NO: 867 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaucaagugccuaaucauc

SEQ ID NO: 868 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauaucauaccccuaaucauc

SEQ ID NO: 869 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaucaauccccuaaucauc

SEQ ID NO: 870 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauauccauacccuaaucauc

SEQ ID NO: 871 - RNA, 100 nt, Artificial Sequence
  Description of Artificial Sequence Synthetic polynucleotide
gauauugugacauaucucugcacucaucacacaaaagaugcaaaucuucuauagcugugucagggagggggcauuuuaacauaucguucaccuaaucauc

SEQ ID NO: 872 - RNA, 100 nt, Artificial Sequence
  Description of Artificial Sequence Synthetic polynucleotide
gauauugugacauaucucugcacucaucacacaaaagaugcaaaucuucuauagacauaucagggagggggcauuucaauauaucaccuuccuaaucauc

SEQ ID NO: 873 - RNA, 100 nt, Artificial Sequence
  Description of Artificial Sequence Synthetic polynucleotide
gauauugugacauaucucugcacucaucacacaaaagaugcaaaucuucuauagagcguucagggagggggcauucuaauauauccauuaucuaaucauc

SEQ ID NO: 874 - RNA, 100 nt, Artificial Sequence
  Description of Artificial Sequence Synthetic polynucleotide
gauauugugacauaucucugcacucaucacacaaaagaugcaaaucuucuauaguugugucagggagggggcaucuuaauauaucaggcuucuaaucauc

SEQ ID NO: 875 - RNA, 100 nt, Artificial Sequence
  Description of Artificial Sequence Synthetic polynucleotide
gauauugugacauaucucugcacucaucacacaaaagaugcaaaucuucuauagaacgaucagggagggggcauuuuaauauaucggcgcucuaaucauc

SEQ ID NO: 876 - RNA, 100 nt, Artificial Sequence
  Description of Artificial Sequence Synthetic polynucleotide
gauauugugacauaucucugcacucaucacacaaaagaugcaaaucuucuauaucacggacagggagggggcauuuuaacauaucguucaucuaaucauc

SEQ ID NO: 877 - RNA, 100 nt, Artificial Sequence
  Description of Artificial Sequence Synthetic polynucleotide
gauauugugacauaucucugcacucaucacacaaaagaugcaaaucuucuauaagcaggacagggagggggcauuucaauauaucaagucccuaaucauc

SEQ ID NO: 878 - RNA, 100 nt, Artificial Sequence
  Description of Artificial Sequence Synthetic polynucleotide
gauauugugacauaucucugcacucaucacacaaaagaugcaaaucuucuauacaacaaacagggagggggcauucuaauauauccacuuucuaaucauc

SEQ ID NO: 879 - RNA, 100 nt, Artificial Sequence
  Description of Artificial Sequence Synthetic polynucleotide
gauauugugacauaucucugcacucaucacacaaaagaugcaaaucuucuauacucagaacagggagggggcaucuuaauauaucauaucccuaaucauc

SEQ ID NO: 880 - RNA, 100 nt, Artificial Sequence
  Description of Artificial Sequence Synthetic polynucleotide
gauauugugacauaucucugcacucaucacacaaaagaugcaaaucuucuauacucgagacagggagggggcauuuuaauauaucaccuuccuaaucauc

SEQ ID NO: 881 - RNA, 100 nt, Artificial Sequence
  Description of Artificial Sequence Synthetic polynucleotide
gauauugugacauaucucugcacucaucacacaaaagaugcaaaucuucuauuucaaacacagggagggggcauuuuaacauaucacaauccuaaucauc

SEQ ID NO: 882 - RNA, 100 nt, Artificial Sequence
  Description of Artificial Sequence Synthetic polynucleotide
gauauugugacauaucucugcacucaucacacaaaagaugcaaaucuucuaucacuagcacagggagggggcauuucaauauaucguaacucuaaucauc

SEQ ID NO: 883 - RNA, 100 nt, Artificial Sequence
  Description of Artificial Sequence Synthetic polynucleotide
gauauugugacauaucucugcacucaucacacaaaagaugcaaaucuucuauuucugacacagggagggggcauucuaauauauccacauucuaaucauc

SEQ ID NO: 884 - RNA, 100 nt, Artificial Sequence
  Description of Artificial Sequence Synthetic polynucleotide
gauauugugacauaucucugcacucaucacacaaaagaugcaaaucuucuauccuccacacagggagggggcaucuuaauauaucgguuaucuaaucauc

SEQ ID NO: 885 - RNA, 100 nt, Artificial Sequence
  Description of Artificial Sequence Synthetic polynucleotide
gauauugugacauaucucugcacucaucacacaaaagaugcaaaucuucuauccaacgcacagggagggggcauuuuaauauaucggaacccuaaucauc

SEQ ID NO: 886 - RNA, 100 nt, Artificial Sequence
  Description of Artificial Sequence Synthetic polynucleotide
gauauugugacauaucucugcacucaucacacaaaagaugcaaaucuucuaguccagccacagggagggggcauuuuaacauaucaggcuccuaaucauc

SEQ ID NO: 887 - RNA, 100 nt, Artificial Sequence
  Description of Artificial Sequence Synthetic polynucleotide
gauauugugacauaucucugcacucaucacacaaaagaugcaaaucuucuaaauuucccacagggagggggcauuucaauauaucggcccucuaaucauc

SEQ ID NO: 888 - RNA, 100 nt, Artificial Sequence
  Description of Artificial Sequence Synthetic polynucleotide
gauauugugacauaucucugcacucaucacacaaaagaugcaaaucuucuaguauccccacagggagggggcauucuaauauaucaggccucuaaucauc

SEQ ID NO: 889 - RNA, 100 nt, Artificial Sequence
  Description of Artificial Sequence Synthetic polynucleotide
gauauugugacauaucucugcacucaucacacaaaagaugcaaaucuucuaaaaauaccacagggagggggcaucuuaauauaucggccuccuaaucauc

SEQ ID NO: 890 - RNA, 100 nt, Artificial Sequence
  Description of Artificial Sequence Synthetic polynucleotide
gauauugugacauaucucugcacucaucacacaaaagaugcaaaucuucuaaguaaaccacagggagggggcauuuuaauauaucagaccccuaaucauc

SEQ ID NO: 891 - RNA, 100 nt, Artificial Sequence
  Description of Artificial Sequence Synthetic polynucleotide
gauauugugacauaucucugcacucaucacacaaaagaugcaaaucuucucuuuuuuccacagggagggggcauuuuaacauaucauacuccuaaucauc

SEQ ID NO: 892 - RNA, 100 nt, Artificial Sequence
  Description of Artificial Sequence Synthetic polynucleotide
gauauugugacauaucucugcacucaucacacaaaagaugcaaaucuucuugaccuuccacagggagggggcauuucaauauaucaaucgccuaaucauc

SEQ ID NO: 893 - RNA, 100 nt, Artificial Sequence
  Description of Artificial Sequence Synthetic polynucleotide
gauauugugacauaucucugcacucaucacacaaaagaugcaaaucuucuccguacuccacagggagggggcauucuaauauaucaauacucuaaucauc

SEQ ID NO: 894 - RNA, 100 nt, Artificial Sequence
  Description of Artificial Sequence Synthetic polynucleotide
gauauugugacauaucucugcacucaucacacaaaagaugcaaaucuucuccuuucuccacagggagggggcaucuuaauauaucaagcuccuaaucauc

SEQ ID NO: 895 - RNA, 100 nt, Artificial Sequence
  Description of Artificial Sequence Synthetic polynucleotide
gauauugugacauaucucugcacucaucacacaaaagaugcaaaucuucucgacgcuccacagggagggggcauuuuaauauaucgacuuccuaaucauc

SEQ ID NO: 896 - RNA, 100 nt, Artificial Sequence
  Description of Artificial Sequence Synthetic polynucleotide
gauauugugacauaucucugcacucaucacacaaaagaugcaaaucuucaacgccauccacagggagggggcauuuuaacauauccaaagucuaaucauc

SEQ ID NO: 897 - RNA, 100 nt, Artificial Sequence
  Description of Artificial Sequence Synthetic polynucleotide
gauauugugacauaucucugcacucaucacacaaaagaugcaaaucuuccaacgcauccacagggagggggcauuucaauauaucaccuuucuaaucauc

SEQ ID NO: 898 - RNA, 100 nt, Artificial Sequence
  Description of Artificial Sequence Synthetic polynucleotide
gauauugugacauaucucugcacucaucacacaaaagaugcaaaucuucagcgcaauccacagggagggggcauucuaauauaucagcauccuaaucauc

SEQ ID NO: 899 - RNA, 100 nt, Artificial Sequence
  Description of Artificial Sequence Synthetic polynucleotide
gauauugugacauaucucugcacucaucacacaaaagaugcaaaucuucgugucaauccacagggagggggcaucuuaauauauccaaauucuaaucauc

SEQ ID NO: 900 - RNA, 100 nt, Artificial Sequence
  Description of Artificial Sequence Synthetic polynucleotide
gauauugugacauaucucugcacucaucacacaaaagaugcaaaucuucgaauaaauccacagggagggggcauuuuaauauaucagcuuucuaaucauc

SEQ ID NO: 901 - RNA, 100 nt, Artificial Sequence
  Description of Artificial Sequence Synthetic polynucleotide
gauauugugacauaucucugcacucaucacacaaaagaugcaaaucuuacgaaagauccacagggagggggcauuuuaacauaucggaauccuaaucauc

SEQ ID NO: 902 - RNA, 100 nt, Artificial Sequence
  Description of Artificial Sequence Synthetic polynucleotide
gauauugugacauaucucugcacucaucacacaaaagaugcaaaucuugguccugauccacagggagggggcauuucaauauaucguaauucuaaucauc

SEQ ID NO: 903 - RNA, 100 nt, Artificial Sequence
  Description of Artificial Sequence Synthetic polynucleotide
gauauugugacauaucucugcacucaucacacaaaagaugcaaaucuugcaccugauccacagggagggggcauucuaauauaucgacacccuaaucauc

SEQ ID NO: 904 - RNA, 100 nt, Artificial Sequence
  Description of Artificial Sequence Synthetic polynucleotide
gauauugugacauaucucugcacucaucacacaaaagaugcaaaucuuaggaaagauccacagggagggggcaucuuaauauaucagguaucuaaucauc

SEQ ID NO: 905 - RNA, 100 nt, Artificial Sequence
  Description of Artificial Sequence Synthetic polynucleotide
gauauugugacauaucucugcacucaucacacaaaagaugcaaaucuuacgacagauccacagggagggggcauuuuaauauaucguacaccuaaucauc

SEQ ID NO: 906 - RNA, 100 nt, Artificial Sequence
  Description of Artificial Sequence Synthetic polynucleotide
gauauugugacauaucucugcacucaucacacaaaagaugcaaaucugugggcggauccacagggagggggcauuuuaacauaucagcucccuaaucauc

SEQ ID NO: 907 - RNA, 100 nt, Artificial Sequence
  Description of Artificial Sequence Synthetic polynucleotide
gauauugugacauaucucugcacucaucacacaaaagaugcaaaucucaucgcggauccacagggagggggcauuucaauauaucgguacccuaaucauc

SEQ ID NO: 908 - RNA, 100 nt, Artificial Sequence
  Description of Artificial Sequence Synthetic polynucleotide
gauauugugacauaucucugcacucaucacacaaaagaugcaaaucuagccuuggauccacagggagggggcauucuaauauaucgggcuccuaaucauc

SEQ ID NO: 909 - RNA, 100 nt, Artificial Sequence
  Description of Artificial Sequence Synthetic polynucleotide
gauauugugacauaucucugcacucaucacacaaaagaugcaaaucugaacacggauccacagggagggggcaucuuaauauaucaagcuucuaaucauc

SEQ ID NO: 910 - RNA, 100 nt, Artificial Sequence
  Description of Artificial Sequence Synthetic polynucleotide
gauauugugacauaucucugcacucaucacacaaaagaugcaaaucugcgcguggauccacagggagggggcauuuuaauauaucgccuuccuaaucauc

SEQ ID NO: 911 - RNA, 100 nt, Artificial Sequence
  Description of Artificial Sequence Synthetic polynucleotide
gauauugugacauaucucugcacucaucacacaaaagaugcaaauccuggagaggauccacagggagggggcauuuuaacauaucaauucucuaaucauc

SEQ ID NO: 912 - RNA, 100 nt, Artificial Sequence
  Description of Artificial Sequence Synthetic polynucleotide
gauauugugacauaucucugcacucaucacacaaaagaugcaaaucgcaaacaggauccacagggagggggcauuucaauauaucguaucucuaaucauc

SEQ ID NO: 913 - RNA, 100 nt, Artificial Sequence
  Description of Artificial Sequence Synthetic polynucleotide
gauauugugacauaucucugcacucaucacacaaaagaugcaaaucggugcgaggauccacagggagggggcauucuaauauaucaaacuucuaaucauc

SEQ ID NO: 914 - RNA, 100 nt, Artificial Sequence
  Description of Artificial Sequence Synthetic polynucleotide
gauauugugacauaucucugcacucaucacacaaaagaugcaaaucgaggaaaggauccacagggagggggcaucuuaauauaucaggccucuaaucauc

SEQ ID NO: 915 - RNA, 100 nt, Artificial Sequence
  Description of Artificial Sequence Synthetic polynucleotide
gauauugugacauaucucugcacucaucacacaaaagaugcaaauccggaagaggauccacagggagggggcauuuuaauauaucaaaccccuaaucauc

SEQ ID NO: 916 - RNA, 100 nt, Artificial Sequence
  Description of Artificial Sequence Synthetic polynucleotide
gauauugugacauaucucugcacucaucacacaaaagaugcaaauagcgccuaggauccacagggagggggcauuuuaacauaucggcuuucuaaucauc

SEQ ID NO: 917 - RNA, 100 nt, Artificial Sequence
  Description of Artificial Sequence Synthetic polynucleotide
gauauugugacauaucucugcacucaucacacaaaagaugcaaauaaguauuaggauccacagggagggggcauuucaauauaucaacauucuaaucauc

SEQ ID NO: 918 - RNA, 100 nt, Artificial Sequence
  Description of Artificial Sequence Synthetic polynucleotide
gauauugugacauaucucugcacucaucacacaaaagaugcaaauagaauuuaggauccacagggagggggcaucuuaauauaucagcuuucuaaucauc

SEQ ID NO: 919 - RNA, 100 nt, Artificial Sequence
  Description of Artificial Sequence Synthetic polynucleotide
gauauugugacauaucucugcacucaucacacaaaagaugcaaauaguaguuaggauccacagggagggggcauuuuaauauaucagaucucuaaucauc

SEQ ID NO: 920 - RNA, 100 nt, Artificial Sequence
  Description of Artificial Sequence Synthetic polynucleotide
gauauugugacauaucucugcacucaucacacaaaagaugcaaaggccacauaggauccacagggagggggcauuucaauauaucaagucccuaaucauc

SEQ ID NO: 921 - RNA, 100 nt, Artificial Sequence
  Description of Artificial Sequence Synthetic polynucleotide
gauauugugacauaucucugcacucaucacacaaaagaugcaaaauagugauaggauccacagggagggggcauucuaauauauccaccgccuaaucauc

SEQ ID NO: 922 - RNA, 100 nt, Artificial Sequence
  Description of Artificial Sequence Synthetic polynucleotide
gauauugugacauaucucugcacucaucacacaaaagaugcaaagaggacauaggauccacagggagggggcaucuuaauauaucggucaucuaaucauc

SEQ ID NO: 923 - RNA, 100 nt, Artificial Sequence
  Description of Artificial Sequence Synthetic polynucleotide
gauauugugacauaucucugcacucaucacacaaaagaugcaaagggcaaauaggauccacagggagggggcauuuuaauauaucaggcaucuaaucauc

SEQ ID NO: 924 - RNA, 100 nt, Artificial Sequence
  Description of Artificial Sequence Synthetic polynucleotide
gauauugugacauaucucugcacucaucacacaaaagaugcaacgaccauauaggauccacagggagggggcauuuuaacauauccaaauccuaaucauc

SEQ ID NO: 925 - RNA, 100 nt, Artificial Sequence
  Description of Artificial Sequence Synthetic polynucleotide
gauauugugacauaucucugcacucaucacacaaaagaugcaaccauaauauaggauccacagggagggggcauuucaauauaucagucgucuaaucauc

SEQ ID NO: 926 - RNA, 100 nt, Artificial Sequence
  Description of Artificial Sequence Synthetic polynucleotide
gauauugugacauaucucugcacucaucacacaaaagaugcaacgaauauauaggauccacagggagggggcauucuaauauaucgauacccuaaucauc

SEQ ID NO: 927 - RNA, 100 nt, Artificial Sequence
  Description of Artificial Sequence Synthetic polynucleotide
gauauugugacauaucucugcacucaucacacaaaagaugcaauuagcauauaggauccacagggagggggcaucuuaauauaucgccauucuaaucauc

SEQ ID NO: 928 - RNA, 100 nt, Artificial Sequence
  Description of Artificial Sequence Synthetic polynucleotide
gauauugugacauaucucugcacucaucacacaaaagaugcaacgacgguauaggauccacagggagggggcauuuuaauauaucguguuucuaaucauc

SEQ ID NO: 929 - RNA, 100 nt, Artificial Sequence
  Description of Artificial Sequence Synthetic polynucleotide
gauauugugacauaucucugcacucaucacacaaaagaugcaccuagacuauaggauccacagggagggggcauuuuaacauaucggaauucuaaucauc

SEQ ID NO: 930 - RNA, 100 nt, Artificial Sequence
  Description of Artificial Sequence Synthetic polynucleotide
gauauugugacauaucucugcacucaucacacaaaagaugcauuggcccuauaggauccacagggagggggcauuucaauauauccaacaccuaaucauc

SEQ ID NO: 931 - RNA, 100 nt, Artificial Sequence
  Description of Artificial Sequence Synthetic polynucleotide
gauauugugacauaucucugcacucaucacacaaaagaugcacggacacuauaggauccacagggagggggcauucuaauauaucagauuucuaaucauc

SEQ ID NO: 932 - RNA, 100 nt, Artificial Sequence
  Description of Artificial Sequence Synthetic polynucleotide
gauauugugacauaucucugcacucaucacacaaaagaugcauccauacuauaggauccacagggagggggcaucuuaauauauccaacuucuaaucauc

SEQ ID NO: 933 - RNA, 100 nt, Artificial Sequence
  Description of Artificial Sequence Synthetic polynucleotide
gauauugugacauaucucugcacucaucacacaaaagaugcauucgggcuauaggauccacagggagggggcauuuuaauauaucagguuccuaaucauc

SEQ ID NO: 934 - RNA, 100 nt, Artificial Sequence
  Description of Artificial Sequence Synthetic polynucleotide
gauauugugacauaucucugcacucaucacacaaaagaugcucccuaucuauaggauccacagggagggggcauuuuaacauauccaccuccuaaucauc

SEQ ID NO: 935 - RNA, 100 nt, Artificial Sequence
  Description of Artificial Sequence Synthetic polynucleotide
gauauugugacauaucucugcacucaucacacaaaagaugccugaccucuauaggauccacagggagggggcauuucaauauaucggaacucuaaucauc

SEQ ID NO: 936 - RNA, 100 nt, Artificial Sequence
  Description of Artificial Sequence Synthetic polynucleotide
gauauugugacauaucucugcacucaucacacaaaagaugcucgcugucuauaggauccacagggagggggcauucuaauauaucguacuccuaaucauc

SEQ ID NO: 937 - RNA, 100 nt, Artificial Sequence
  Description of Artificial Sequence Synthetic polynucleotide
gauauugugacauaucucugcacucaucacacaaaagaugcuugcacucuauaggauccacagggagggggcaucuuaauauaucggcuuucuaaucauc

SEQ ID NO: 938 - RNA, 100 nt, Artificial Sequence
  Description of Artificial Sequence Synthetic polynucleotide
gauauugugacauaucucugcacucaucacacaaaagaugcccagaaucuauaggauccacagggagggggcauuuuaauauaucaguaaucuaaucauc

SEQ ID NO: 939 - RNA, 100 nt, Artificial Sequence
  Description of Artificial Sequence Synthetic polynucleotide
gauauugugacauaucucugcacucaucacacaaaagaugaauuuauucuauaggauccacagggagggggcauuuuaacauaucgauacucuaaucauc

SEQ ID NO: 940 - RNA, 100 nt, Artificial Sequence
  Description of Artificial Sequence Synthetic polynucleotide
gauauugugacauaucucugcacucaucacacaaaagaugguugcguucuauaggauccacagggagggggcauuucaauauaucacauaccuaaucauc

SEQ ID NO: 941 - RNA, 100 nt, Artificial Sequence
  Description of Artificial Sequence Synthetic polynucleotide
gauauugugacauaucucugcacucaucacacaaaagaugaccgcauucuauaggauccacagggagggggcaucuuaauauaucaggugccuaaucauc

SEQ ID NO: 942 - RNA, 100 nt, Artificial Sequence
  Description of Artificial Sequence Synthetic polynucleotide
gauauugugacauaucucugcacucaucacacaaaagaugaacgcauucuauaggauccacagggagggggcauuuuaauauaucgccccccuaaucauc

SEQ ID NO: 943 - RNA, 100 nt, Artificial Sequence
  Description of Artificial Sequence Synthetic polynucleotide
gauauugugacauaucucugcacucaucacacaaaagauaccacccuucuauaggauccacagggagggggcauuuuaacauaucauaagccuaaucauc

SEQ ID NO: 944 - RNA, 100 nt, Artificial Sequence
  Description of Artificial Sequence Synthetic polynucleotide
gauauugugacauaucucugcacucaucacacaaaagauuaugcccuucuauaggauccacagggagggggcauuucaauauaucgaauuccuaaucauc

SEQ ID NO: 945 - RNA, 100 nt, Artificial Sequence
  Description of Artificial Sequence Synthetic polynucleotide
gauauugugacauaucucugcacucaucacacaaaagauugaccacuucuauaggauccacagggagggggcauucuaauauaucagcauccuaaucauc

SEQ ID NO: 946 - RNA, 100 nt, Artificial Sequence
  Description of Artificial Sequence Synthetic polynucleotide
gauauugugacauaucucugcacucaucacacaaaagauuagugccuucuauaggauccacagggagggggcaucuuaauauaucaggucccuaaucauc

SEQ ID NO: 947 - RNA, 100 nt, Artificial Sequence
  Description of Artificial Sequence Synthetic polynucleotide
gauauugugacauaucucugcacucaucacacaaaagauuuuauacuucuauaggauccacagggagggggcauuuuaauauaucggguuccuaaucauc

SEQ ID NO: 948 - RNA, 100 nt, Artificial Sequence
  Description of Artificial Sequence Synthetic polynucleotide
gauauugugacauaucucugcacucaucacacaaaagacuuaccucuucuauaggauccacagggagggggcauuuuaacauaucggauaucuaaucauc

SEQ ID NO: 949 - RNA, 100 nt, Artificial Sequence
  Description of Artificial Sequence Synthetic polynucleotide
gauauugugacauaucucugcacucaucacacaaaagacuaguuucuucuauaggauccacagggagggggcauuucaauauaucagcuaucuaaucauc

SEQ ID NO: 950 - RNA, 100 nt, Artificial Sequence
  Description of Artificial Sequence Synthetic polynucleotide
gauauugugacauaucucugcacucaucacacaaaagacaaccuucuucuauaggauccacagggagggggcauucuaauauaucgagacccuaaucauc

SEQ ID NO: 951 - RNA, 100 nt, Artificial Sequence
  Description of Artificial Sequence Synthetic polynucleotide
gauauugugacauaucucugcacucaucacacaaaagaaaucgcucuucuauaggauccacagggagggggcaucuuaauauauccacucucuaaucauc

SEQ ID NO: 952 - RNA, 100 nt, Artificial Sequence
  Description of Artificial Sequence Synthetic polynucleotide
gauauugugacauaucucugcacucaucacacaaaagaacauuuucuucuauaggauccacagggagggggcauuuuaauauaucagcacucuaaucauc

SEQ ID NO: 953 - RNA, 100 nt, Artificial Sequence
  Description of Artificial Sequence Synthetic polynucleotide
gauauugugacauaucucugcacucaucacacaaaagcccuauaucuucuauaggauccacagggagggggcauuuuaacauaucgguuuccuaaucauc

SEQ ID NO: 954 - RNA, 100 nt, Artificial Sequence
  Description of Artificial Sequence Synthetic polynucleotide
gauauugugacauaucucugcacucaucacacaaaaguacucuaucuucuauaggauccacagggagggggcauuucaauauaucgagccucuaaucauc

SEQ ID NO: 955 - RNA, 100 nt, Artificial Sequence
  Description of Artificial Sequence Synthetic polynucleotide
gauauugugacauaucucugcacucaucacacaaaagcacguuaucuucuauaggauccacagggagggggcauucuaauauaucgugccccuaaucauc

SEQ ID NO: 956 - RNA, 100 nt, Artificial Sequence
  Description of Artificial Sequence Synthetic polynucleotide
gauauugugacauaucucugcacucaucacacaaaagcacacuaucuucuauaggauccacagggagggggcaucuuaauauaucgggccccuaaucauc

SEQ ID NO: 957 - RNA, 100 nt, Artificial Sequence
  Description of Artificial Sequence Synthetic polynucleotide
gauauugugacauaucucugcacucaucacacaaaagcacuccaucuucuauaggauccacagggagggggcauuuuaauauaucagguuccuaaucauc

SEQ ID NO: 958 - RNA, 100 nt, Artificial Sequence
  Description of Artificial Sequence Synthetic polynucleotide
gauauugugacauaucucugcacucaucacacaaaacugaacaaucuucuauaggauccacagggagggggcauuucaauauaucgacagccuaaucauc

SEQ ID NO: 959 - RNA, 100 nt, Artificial Sequence
  Description of Artificial Sequence Synthetic polynucleotide
gauauugugacauaucucugcacucaucacacaaaaucgucuaaucuucuauaggauccacagggagggggcauucuaauauaucgguuaucuaaucauc

SEQ ID NO: 960 - RNA, 100 nt, Artificial Sequence
  Description of Artificial Sequence Synthetic polynucleotide
gauauugugacauaucucugcacucaucacacaaaaucgaccaaucuucuauaggauccacagggagggggcaucuuaauauaucggaauccuaaucauc

SEQ ID NO: 961 - RNA, 100 nt, Artificial Sequence
  Description of Artificial Sequence Synthetic polynucleotide
gauauugugacauaucucugcacucaucacacaaaacccaguaaucuucuauaggauccacagggagggggcauuuuaauauaucgcaucccuaaucauc

SEQ ID NO: 962 - RNA, 100 nt, Artificial Sequence
  Description of Artificial Sequence Synthetic polynucleotide
gauauugugacauaucucugcacucaucacacaaaucgcagaaaucuucuauaggauccacagggagggggcauuuuaacauaucgggcaccuaaucauc

SEQ ID NO: 963 - RNA, 100 nt, Artificial Sequence
  Description of Artificial Sequence Synthetic polynucleotide
gauauugugacauaucucugcacucaucacacaaauuuuuaaaaucuucuauaggauccacagggagggggcauuucaauauaucggaagccuaaucauc

SEQ ID NO: 964 - RNA, 100 nt, Artificial Sequence
  Description of Artificial Sequence Synthetic polynucleotide
gauauugugacauaucucugcacucaucacacaaauaaauaaaaucuucuauaggauccacagggagggggcauucuaauauaucagauuccuaaucauc

SEQ ID NO: 965 - RNA, 100 nt, Artificial Sequence
  Description of Artificial Sequence Synthetic polynucleotide
gauauugugacauaucucugcacucaucacacaaauauuuaaaaucuucuauaggauccacagggagggggcaucuuaauauaucauacgucuaaucauc

SEQ ID NO: 966 - RNA, 100 nt, Artificial Sequence
  Description of Artificial Sequence Synthetic polynucleotide
gauauugugacauaucucugcacucaucacacaaacauccgaaaucuucuauaggauccacagggagggggcauuuuaauauauccacagccuaaucauc

SEQ ID NO: 967 - RNA, 100 nt, Artificial Sequence
  Description of Artificial Sequence Synthetic polynucleotide
gauauugugacauaucucugcacucaucacacaaugcucccaaaucuucuauaggauccacagggagggggcauuuuaacauaucauaagucuaaucauc

SEQ ID NO: 968 - RNA, 100 nt, Artificial Sequence
  Description of Artificial Sequence Synthetic polynucleotide
gauauugugacauaucucugcacucaucacacaacuuuuucaaaucuucuauaggauccacagggagggggcauuucaauauaucauauuucuaaucauc

SEQ ID NO: 969 - RNA, 100 nt, Artificial Sequence
  Description of Artificial Sequence Synthetic polynucleotide
gauauugugacauaucucugcacucaucacacaaucaacucaaaucuucuauaggauccacagggagggggcauucuaauauaucggaauucuaaucauc

SEQ ID NO: 970 - RNA, 100 nt, Artificial Sequence
  Description of Artificial Sequence Synthetic polynucleotide
gauauugugacauaucucugcacucaucacacaacuacaccaaaucuucuauaggauccacagggagggggcaucuuaauauaucaaaauucuaaucauc

SEQ ID NO: 971 - RNA, 100 nt, Artificial Sequence
  Description of Artificial Sequence Synthetic polynucleotide
gauauugugacauaucucugcacucaucacacacaaaucgcaaaucuucuauaggauccacagggagggggcauuuuaacauauccauuuucuaaucauc

SEQ ID NO: 972 - RNA, 100 nt, Artificial Sequence
  Description of Artificial Sequence Synthetic polynucleotide
gauauugugacauaucucugcacucaucacacacgcaaagcaaaucuucuauaggauccacagggagggggcauuucaauauauccacgcccuaaucauc

SEQ ID NO: 973 - RNA, 100 nt, Artificial Sequence
  Description of Artificial Sequence Synthetic polynucleotide
gauauugugacauaucucugcacucaucacacaucacaagcaaaucuucuauaggauccacagggagggggcauucuaauauaucgucaaucuaaucauc

SEQ ID NO: 974 - RNA, 100 nt, Artificial Sequence
  Description of Artificial Sequence Synthetic polynucleotide
gauauugugacauaucucugcacucaucacacaccgcuggcaaaucuucuauaggauccacagggagggggcaucuuaauauaucguauuccuaaucauc

SEQ ID NO: 975 - RNA, 100 nt, Artificial Sequence
  Description of Artificial Sequence Synthetic polynucleotide
gauauugugacauaucucugcacucaucacacacacucagcaaaucuucuauaggauccacagggagggggcauuuuaauauaucgaucuucuaaucauc

SEQ ID NO: 976 - RNA, 100 nt, Artificial Sequence
  Description of Artificial Sequence Synthetic polynucleotide
gauauugugacauaucucugcacucaucacaccgucguugcaaaucuucuauaggauccacagggagggggcauuuuaacauaucguguuucuaaucauc

SEQ ID NO: 977 - RNA, 100 nt, Artificial Sequence
  Description of Artificial Sequence Synthetic polynucleotide
gauauugugacauaucucugcacucaucacaccuguccugcaaaucuucuauaggauccacagggagggggcauuucaauauaucagcauccuaaucauc

SEQ ID NO: 978 - RNA, 100 nt, Artificial Sequence
  Description of Artificial Sequence Synthetic polynucleotide
gauauugugacauaucucugcacucaucacaccgcuucugcaaaucuucuauaggauccacagggagggggcauucuaauauaucggguuccuaaucauc

SEQ ID NO: 979 - RNA, 100 nt, Artificial Sequence
  Description of Artificial Sequence Synthetic polynucleotide
gauauugugacauaucucugcacucaucacacuaacacugcaaaucuucuauaggauccacagggagggggcaucuuaauauaucaacucccuaaucauc

SEQ ID NO: 980 - RNA, 100 nt, Artificial Sequence
  Description of Artificial Sequence Synthetic polynucleotide
gauauugugacauaucucugcacucaucacacuuuacuugcaaaucuucuauaggauccacagggagggggcauuuuaauauaucgugcuucuaaucauc

SEQ ID NO: 981 - RNA, 100 nt, Artificial Sequence
  Description of Artificial Sequence Synthetic polynucleotide
gauauugugacauaucucugcacucaucacagucaucaugcaaaucuucuauaggauccacagggagggggcauuucaauauaucaaaaaccuaaucauc

SEQ ID NO: 982 - RNA, 100 nt, Artificial Sequence
  Description of Artificial Sequence Synthetic polynucleotide
gauauugugacauaucucugcacucaucacaaugugaaugcaaaucuucuauaggauccacagggagggggcauucuaauauaucguaucucuaaucauc

SEQ ID NO: 983 - RNA, 100 nt, Artificial Sequence
  Description of Artificial Sequence Synthetic polynucleotide
gauauugugacauaucucugcacucaucacaauuacuaugcaaaucuucuauaggauccacagggagggggcaucuuaauauaucaaaacucuaaucauc

SEQ ID NO: 984 - RNA, 100 nt, Artificial Sequence
  Description of Artificial Sequence Synthetic polynucleotide
gauauugugacauaucucugcacucaucacagcuuaaaugcaaaucuucuauaggauccacagggagggggcauuuuaauauaucaacacccuaaucauc

SEQ ID NO: 985 - RNA, 100 nt, Artificial Sequence
  Description of Artificial Sequence Synthetic polynucleotide
gauauugugacauaucucugcacucaucacuuggucgaugcaaaucuucuauaggauccacagggagggggcauuuuaacauaucgggccucuaaucauc

SEQ ID NO: 986 - RNA, 100 nt, Artificial Sequence
  Description of Artificial Sequence Synthetic polynucleotide
gauauugugacauaucucugcacucaucacugauccgaugcaaaucuucuauaggauccacagggagggggcauuucaauauaucaacauccuaaucauc

SEQ ID NO: 987 - RNA, 100 nt, Artificial Sequence
  Description of Artificial Sequence Synthetic polynucleotide
gauauugugacauaucucugcacucaucacuugcucgaugcaaaucuucuauaggauccacagggagggggcauucuaauauauccacacucuaaucauc

SEQ ID NO: 988 - RNA, 100 nt, Artificial Sequence
  Description of Artificial Sequence Synthetic polynucleotide
gauauugugacauaucucugcacucaucacuguccugaugcaaaucuucuauaggauccacagggagggggcaucuuaauauaucguaagccuaaucauc

SEQ ID NO: 989 - RNA, 100 nt, Artificial Sequence
  Description of Artificial Sequence Synthetic polynucleotide
gauauugugacauaucucugcacucaucacuguaucgaugcaaaucuucuauaggauccacagggagggggcauuuuaauauauccaauuucuaaucauc

SEQ ID NO: 990 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaugaagcuacuaaucauc

SEQ ID NO: 991 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauaugcaaauacuaaucauc

SEQ ID NO: 992 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauauagggcuacuaaucauc

SEQ ID NO: 993 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaugcauacacuaaucauc

SEQ ID NO: 994 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauauagagauacuaaucauc

SEQ ID NO: 995 - RNA, 100 nt, Artificial Sequence
  Description of Artificial Sequence Synthetic polynucleotide
gauauugugacauaucucugcacucaucacacaaaagaugcaaaucuucuauaucgcgaacagggagggggcauuuuaacauauaggcguacuaaucauc

SEQ ID NO: 996 - RNA, 100 nt, Artificial Sequence
  Description of Artificial Sequence Synthetic polynucleotide
gauauugugacauaucucugcacucaucacacaaaagaugcaaaucuucuauaacccuaacagggagggggcauuucaauauaugagaauacuaaucauc

SEQ ID NO: 997 - RNA, 100 nt, Artificial Sequence
  Description of Artificial Sequence Synthetic polynucleotide
gauauugugacauaucucugcacucaucacacaaaagaugcaaaucuucuauaucaguaacagggagggggcaucuuaauauauaaccacacuaaucauc

SEQ ID NO: 998 - RNA, 100 nt, Artificial Sequence
  Description of Artificial Sequence Synthetic polynucleotide
gauauugugacauaucucugcacucaucacacaaaagaugcaaaucuucuauacuaguaacagggagggggcauuuuaauauauagguauacuaaucauc

SEQ ID NO: 999 - RNA, 100 nt, Artificial Sequence
  Description of Artificial Sequence Synthetic polynucleotide
gauauugugacauaucucugcacucaucacacaaaagaugcaaaucuucuaucacuggcacagggagggggcauuuuaacauauggaaccacuaaucauc

SEQ ID NO: 1000 - RNA, 100 nt, Artificial Sequence
  Description of Artificial Sequence Synthetic polynucleotide
gauauugugacauaucucugcacucaucacacaaaagaugcaaaucuucuauuaacagcacagggagggggcauuucaauauauaugcacacuaaucauc

SEQ ID NO: 1001 - RNA, 100 nt, Artificial Sequence
  Description of Artificial Sequence Synthetic polynucleotide
gauauugugacauaucucugcacucaucacacaaaagaugcaaaucuucuauuuaacacacagggagggggcauucuaauauauggaaauacuaaucauc

SEQ ID NO: 1002 - RNA, 100 nt, Artificial Sequence
  Description of Artificial Sequence Synthetic polynucleotide
gauauugugacauaucucugcacucaucacacaaaagaugcaaaucuucuaucccuaacacagggagggggcaucuuaauauauaaggcuacuaaucauc

SEQ ID NO: 1003 - RNA, 100 nt, Artificial Sequence
  Description of Artificial Sequence Synthetic polynucleotide
gauauugugacauaucucugcacucaucacacaaaagaugcaaaucuucuauuagcagcacagggagggggcauuuuaauauaugagaguacuaaucauc

SEQ ID NO: 1004 - RNA, 100 nt, Artificial Sequence
  Description of Artificial Sequence Synthetic polynucleotide
gauauugugacauaucucugcacucaucacacaaaagaugcaaaucuucuaacaaugccacagggagggggcauuucaauauaugcauuuacuaaucauc

SEQ ID NO: 1005 - RNA, 100 nt, Artificial Sequence
  Description of Artificial Sequence Synthetic polynucleotide
gauauugugacauaucucugcacucaucacacaaaagaugcaaaucuucuacuucgcccacagggagggggcauucuaauauaugcauacacuaaucauc

SEQ ID NO: 1006 - RNA, 100 nt, Artificial Sequence
  Description of Artificial Sequence Synthetic polynucleotide
gauauugugacauaucucugcacucaucacacaaaagaugcaaaucuucuaguuaagccacagggagggggcaucuuaauauauauagucacuaaucauc

SEQ ID NO: 1007 - RNA, 100 nt, Artificial Sequence
  Description of Artificial Sequence Synthetic polynucleotide
gauauugugacauaucucugcacucaucacacaaaagaugcaaaucuucuaauuaagccacagggagggggcauuuuaauauauagauacacuaaucauc

SEQ ID NO: 1008 - RNA, 100 nt, Artificial Sequence
  Description of Artificial Sequence Synthetic polynucleotide
gauauugugacauaucucugcacucaucacacaaaagaugcaaaucuucuuuuucuuccacagggagggggcauuuuaacauaugagaguacuaaucauc

SEQ ID NO: 1009 - RNA, 100 nt, Artificial Sequence
  Description of Artificial Sequence Synthetic polynucleotide
gauauugugacauaucucugcacucaucacacaaaagaugcaaaucuucuuauuguuccacagggagggggcauuucaauauauauaacuacuaaucauc

SEQ ID NO: 1010 - RNA, 100 nt, Artificial Sequence
  Description of Artificial Sequence Synthetic polynucleotide
gauauugugacauaucucugcacucaucacacaaaagaugcaaaucuucuugacacuccacagggagggggcauucuaauauauaaacccacuaaucauc

SEQ ID NO: 1011 - RNA, 100 nt, Artificial Sequence
  Description of Artificial Sequence Synthetic polynucleotide
gauauugugacauaucucugcacucaucacacaaaagaugcaaaucuucucuuaucuccacagggagggggcaucuuaauauaugaaccuacuaaucauc

SEQ ID NO: 1012 - RNA, 100 nt, Artificial Sequence
  Description of Artificial Sequence Synthetic polynucleotide
gauauugugacauaucucugcacucaucacacaaaagaugcaaaucuucuuguaccuccacagggagggggcauuuuaauauauaaccacacuaaucauc

SEQ ID NO: 1013 - RNA, 100 nt, Artificial Sequence
  Description of Artificial Sequence Synthetic polynucleotide
gauauugugacauaucucugcacucaucacacaaaagaugcaaaucuucacuucaauccacagggagggggcauuuuaacauauauuaccacuaaucauc

SEQ ID NO: 1014 - RNA, 100 nt, Artificial Sequence
  Description of Artificial Sequence Synthetic polynucleotide
gauauugugacauaucucugcacucaucacacaaaagaugcaaaucuuccagucuauccacagggagggggcauuucaauauaugauaccacuaaucauc

SEQ ID NO: 1015 - RNA, 100 nt, Artificial Sequence
  Description of Artificial Sequence Synthetic polynucleotide
gauauugugacauaucucugcacucaucacacaaaagaugcaaaucuuccccaccauccacagggagggggcauucuaauauaugggacuacuaaucauc

SEQ ID NO: 1016 - RNA, 100 nt, Artificial Sequence
  Description of Artificial Sequence Synthetic polynucleotide
gauauugugacauaucucugcacucaucacacaaaagaugcaaaucuucgacuccauccacagggagggggcaucuuaauauaugcauccacuaaucauc

SEQ ID NO: 1017 - RNA, 100 nt, Artificial Sequence
  Description of Artificial Sequence Synthetic polynucleotide
gauauugugacauaucucugcacucaucacacaaaagaugcaaaucuucaccgaaauccacagggagggggcauuuuaauauaugcauauacuaaucauc

SEQ ID NO: 1018 - RNA, 100 nt, Artificial Sequence
  Description of Artificial Sequence Synthetic polynucleotide
gauauugugacauaucucugcacucaucacacaaaagaugcaaaucuuagucucgauccacagggagggggcauuucaauauaugaacccacuaaucauc

SEQ ID NO: 1019 - RNA, 100 nt, Artificial Sequence
  Description of Artificial Sequence Synthetic polynucleotide
gauauugugacauaucucugcacucaucacacaaaagaugcaaaucuuaauccugauccacagggagggggcauucuaauauauaacaacacuaaucauc

SEQ ID NO: 1020 - RNA, 100 nt, Artificial Sequence
  Description of Artificial Sequence Synthetic polynucleotide
gauauugugacauaucucugcacucaucacacaaaagaugcaaaucuuggcaccgauccacagggagggggcaucuuaauauauagaguuacuaaucauc

SEQ ID NO: 1021 - RNA, 100 nt, Artificial Sequence
  Description of Artificial Sequence Synthetic polynucleotide
gauauugugacauaucucugcacucaucacacaaaagaugcaaaucuugaucaagauccacagggagggggcauuuuaauauauagacguacuaaucauc

SEQ ID NO: 1022 - RNA, 100 nt, Artificial Sequence
  Description of Artificial Sequence Synthetic polynucleotide
gauauugugacauaucucugcacucaucacacaaaagaugcaaaucugacaauggauccacagggagggggcauuuuaacauauaaccauacuaaucauc

SEQ ID NO: 1023 - RNA, 100 nt, Artificial Sequence
  Description of Artificial Sequence Synthetic polynucleotide
gauauugugacauaucucugcacucaucacacaaaagaugcaaaucuagcgcuggauccacagggagggggcauuucaauauauaacauuacuaaucauc

SEQ ID NO: 1024 - RNA, 100 nt, Artificial Sequence
  Description of Artificial Sequence Synthetic polynucleotide
gauauugugacauaucucugcacucaucacacaaaagaugcaaaucucacgacggauccacagggagggggcauucuaauauaugcaguuacuaaucauc

SEQ ID NO: 1025 - RNA, 100 nt, Artificial Sequence
  Description of Artificial Sequence Synthetic polynucleotide
gauauugugacauaucucugcacucaucacacaaaagaugcaaaucuguuuccggauccacagggagggggcaucuuaauauauggagccacuaaucauc

SEQ ID NO: 1026 - RNA, 100 nt, Artificial Sequence
  Description of Artificial Sequence Synthetic polynucleotide
gauauugugacauaucucugcacucaucacacaaaagaugcaaaucuggcuuuggauccacagggagggggcauuuuaauauauacgagcacuaaucauc

SEQ ID NO: 1027 - RNA, 100 nt, Artificial Sequence
  Description of Artificial Sequence Synthetic polynucleotide
gauauugugacauaucucugcacucaucacacaaaagaugcaaaucgacagaaggauccacagggagggggcauuuuaacauauaaagauacuaaucauc

SEQ ID NO: 1028 - RNA, 100 nt, Artificial Sequence
  Description of Artificial Sequence Synthetic polynucleotide
gauauugugacauaucucugcacucaucacacaaaagaugcaaauccuagagaggauccacagggagggggcauuucaauauauauaaauacuaaucauc

SEQ ID NO: 1029 - RNA, 100 nt, Artificial Sequence
  Description of Artificial Sequence Synthetic polynucleotide
gauauugugacauaucucugcacucaucacacaaaagaugcaaauccugcggaggauccacagggagggggcauucuaauauauaagacuacuaaucauc

SEQ ID NO: 1030 - RNA, 100 nt, Artificial Sequence
  Description of Artificial Sequence Synthetic polynucleotide
gauauugugacauaucucugcacucaucacacaaaagaugcaaauccgaucgaggauccacagggagggggcaucuuaauauaugguuauacuaaucauc

SEQ ID NO: 1031 - RNA, 100 nt, Artificial Sequence
  Description of Artificial Sequence Synthetic polynucleotide
gauauugugacauaucucugcacucaucacacaaaagaugcaaaucaaguucaggauccacagggagggggcauuuuaauauauagagcuacuaaucauc

SEQ ID NO: 1032 - RNA, 100 nt, Artificial Sequence
  Description of Artificial Sequence Synthetic polynucleotide
gauauugugacauaucucugcacucaucacacaaaagaugcaaauaaaaccuaggauccacagggagggggcauuuuaacauauacguacacuaaucauc

SEQ ID NO: 1033 - RNA, 100 nt, Artificial Sequence
  Description of Artificial Sequence Synthetic polynucleotide
gauauugugacauaucucugcacucaucacacaaaagaugcaaaugagacuuaggauccacagggagggggcauuucaauauauaaaccuacuaaucauc

SEQ ID NO: 1034 - RNA, 100 nt, Artificial Sequence
  Description of Artificial Sequence Synthetic polynucleotide
gauauugugacauaucucugcacucaucacacaaaagaugcaaaugaaugcuaggauccacagggagggggcaucuuaauauaugcauauacuaaucauc

SEQ ID NO: 1035 - RNA, 100 nt, Artificial Sequence
  Description of Artificial Sequence Synthetic polynucleotide
gauauugugacauaucucugcacucaucacacaaaagaugcaaauggcuuuuaggauccacagggagggggcauuuuaauauauaaacgcacuaaucauc

SEQ ID NO: 1036 - RNA, 100 nt, Artificial Sequence
  Description of Artificial Sequence Synthetic polynucleotide
gauauugugacauaucucugcacucaucacacaaaagaugcaaacgaauaauaggauccacagggagggggcauuuuaacauaugauaucacuaaucauc

SEQ ID NO: 1037 - RNA, 100 nt, Artificial Sequence
  Description of Artificial Sequence Synthetic polynucleotide
gauauugugacauaucucugcacucaucacacaaaagaugcaaaagcaacauaggauccacagggagggggcauuucaauauauggacuuacuaaucauc

SEQ ID NO: 1038 - RNA, 100 nt, Artificial Sequence
  Description of Artificial Sequence Synthetic polynucleotide
gauauugugacauaucucugcacucaucacacaaaagaugcaaaggcauaauaggauccacagggagggggcauucuaauauauaauuauacuaaucauc

SEQ ID NO: 1039 - RNA, 100 nt, Artificial Sequence
  Description of Artificial Sequence Synthetic polynucleotide
gauauugugacauaucucugcacucaucacacaaaagaugcaaacaccggauaggauccacagggagggggcaucuuaauauauggauacacuaaucauc

SEQ ID NO: 1040 - RNA, 100 nt, Artificial Sequence
  Description of Artificial Sequence Synthetic polynucleotide
gauauugugacauaucucugcacucaucacacaaaagaugcaaacgagaaauaggauccacagggagggggcauuuuaauauaugggaccacuaaucauc

SEQ ID NO: 1041 - RNA, 100 nt, Artificial Sequence
  Description of Artificial Sequence Synthetic polynucleotide
gauauugugacauaucucugcacucaucacacaaaagaugcaaccucaguauaggauccacagggagggggcauuuuaacauaugaacacacuaaucauc

SEQ ID NO: 1042 - RNA, 100 nt, Artificial Sequence
  Description of Artificial Sequence Synthetic polynucleotide
gauauugugacauaucucugcacucaucacacaaaagaugcaaccagcguauaggauccacagggagggggcauuucaauauaugcagauacuaaucauc

SEQ ID NO: 1043 - RNA, 100 nt, Artificial Sequence
  Description of Artificial Sequence Synthetic polynucleotide
gauauugugacauaucucugcacucaucacacaaaagaugcaacggaaauauaggauccacagggagggggcauucuaauauauggaaucacuaaucauc

SEQ ID NO: 1044 - RNA, 100 nt, Artificial Sequence
  Description of Artificial Sequence Synthetic polynucleotide
gauauugugacauaucucugcacucaucacacaaaagaugcaauugggauauaggauccacagggagggggcaucuuaauauaugcaacuacuaaucauc

SEQ ID NO: 1045 - RNA, 100 nt, Artificial Sequence
  Description of Artificial Sequence Synthetic polynucleotide
gauauugugacauaucucugcacucaucacacaaaagaugcaacgugcauauaggauccacagggagggggcauuuuaauauauaugcauacuaaucauc

SEQ ID NO: 1046 - RNA, 100 nt, Artificial Sequence
  Description of Artificial Sequence Synthetic polynucleotide
gauauugugacauaucucugcacucaucacacaaaagaugcauuaagccuauaggauccacagggagggggcauuuuaacauauacaaguacuaaucauc

SEQ ID NO: 1047 - RNA, 100 nt, Artificial Sequence
  Description of Artificial Sequence Synthetic polynucleotide
gauauugugacauaucucugcacucaucacacaaaagaugcauuauagcuauaggauccacagggagggggcauuucaauauauauagucacuaaucauc

SEQ ID NO: 1048 - RNA, 100 nt, Artificial Sequence
  Description of Artificial Sequence Synthetic polynucleotide
gauauugugacauaucucugcacucaucacacaaaagaugcacuaggacuauaggauccacagggagggggcauucuaauauauaccauuacuaaucauc

SEQ ID NO: 1049 - RNA, 100 nt, Artificial Sequence
  Description of Artificial Sequence Synthetic polynucleotide
gauauugugacauaucucugcacucaucacacaaaagaugcauaagcccuauaggauccacagggagggggcaucuuaauauaugcauuuacuaaucauc

SEQ ID NO: 1050 - RNA, 100 nt, Artificial Sequence
  Description of Artificial Sequence Synthetic polynucleotide
gauauugugacauaucucugcacucaucacacaaaagaugcucgccgucuauaggauccacagggagggggcauuuuaacauauaguuacacuaaucauc

SEQ ID NO: 1051 - RNA, 100 nt, Artificial Sequence
  Description of Artificial Sequence Synthetic polynucleotide
gauauugugacauaucucugcacucaucacacaaaagaugccgccccucuauaggauccacagggagggggcauuucaauauauagauacacuaaucauc

SEQ ID NO: 1052 - RNA, 100 nt, Artificial Sequence
  Description of Artificial Sequence Synthetic polynucleotide
gauauugugacauaucucugcacucaucacacaaaagaugcucuuggucuauaggauccacagggagggggcauucuaauauauaacaacacuaaucauc

SEQ ID NO: 1053 - RNA, 100 nt, Artificial Sequence
  Description of Artificial Sequence Synthetic polynucleotide
gauauugugacauaucucugcacucaucacacaaaagaugcuccauaucuauaggauccacagggagggggcaucuuaauauauaguagcacuaaucauc

SEQ ID NO: 1054 - RNA, 100 nt, Artificial Sequence
  Description of Artificial Sequence Synthetic polynucleotide
gauauugugacauaucucugcacucaucacacaaaagaugcccuggaucuauaggauccacagggagggggcauuuuaauauaugagaucacuaaucauc

SEQ ID NO: 1055 - RNA, 100 nt, Artificial Sequence
  Description of Artificial Sequence Synthetic polynucleotide
gauauugugacauaucucugcacucaucacacaaaagaugguccuauucuauaggauccacagggagggggcauuuuaacauaugacaucacuaaucauc

SEQ ID NO: 1056 - RNA, 100 nt, Artificial Sequence
  Description of Artificial Sequence Synthetic polynucleotide
gauauugugacauaucucugcacucaucacacaaaagauggucgcauucuauaggauccacagggagggggcauuucaauauauaagaucacuaaucauc

SEQ ID NO: 1057 - RNA, 100 nt, Artificial Sequence
  Description of Artificial Sequence Synthetic polynucleotide
gauauugugacauaucucugcacucaucacacaaaagauggccucguucuauaggauccacagggagggggcauucuaauauauggaguuacuaaucauc

SEQ ID NO: 1058 - RNA, 100 nt, Artificial Sequence
  Description of Artificial Sequence Synthetic polynucleotide
gauauugugacauaucucugcacucaucacacaaaagaugaucggguucuauaggauccacagggagggggcaucuuaauauauacgcacacuaaucauc

SEQ ID NO: 1059 - RNA, 100 nt, Artificial Sequence
  Description of Artificial Sequence Synthetic polynucleotide
gauauugugacauaucucugcacucaucacacaaaagaugguuggguucuauaggauccacagggagggggcauuuuaauauauauacccacuaaucauc

SEQ ID NO: 1060 - RNA, 100 nt, Artificial Sequence
  Description of Artificial Sequence Synthetic polynucleotide
gauauugugacauaucucugcacucaucacacaaaagauaauugacuucuauaggauccacagggagggggcauuuuaacauauacaaguacuaaucauc

SEQ ID NO: 1061 - RNA, 100 nt, Artificial Sequence
  Description of Artificial Sequence Synthetic polynucleotide
gauauugugacauaucucugcacucaucacacaaaagauuauuagcuucuauaggauccacagggagggggcauuucaauauaugaaaguacuaaucauc

SEQ ID NO: 1062 - RNA, 100 nt, Artificial Sequence
  Description of Artificial Sequence Synthetic polynucleotide
gauauugugacauaucucugcacucaucacacaaaagaucuucgccuucuauaggauccacagggagggggcauucuaauauaugaagauacuaaucauc

SEQ ID NO: 1063 - RNA, 100 nt, Artificial Sequence
  Description of Artificial Sequence Synthetic polynucleotide
gauauugugacauaucucugcacucaucacacaaaagauuggucccuucuauaggauccacagggagggggcaucuuaauauaugaagauacuaaucauc

SEQ ID NO: 1064 - RNA, 100 nt, Artificial Sequence
  Description of Artificial Sequence Synthetic polynucleotide
gauauugugacauaucucugcacucaucacacaaaagauaggucgcuucuauaggauccacagggagggggcauuuuaauauauauggcuacuaaucauc

SEQ ID NO: 1065 - RNA, 100 nt, Artificial Sequence
  Description of Artificial Sequence Synthetic polynucleotide
gauauugugacauaucucugcacucaucacacaaaagaccccguucuucuauaggauccacagggagggggcauuuuaacauauggggucacuaaucauc

SEQ ID NO: 1066 - RNA, 100 nt, Artificial Sequence
  Description of Artificial Sequence Synthetic polynucleotide
gauauugugacauaucucugcacucaucacacaaaagacuccucucuucuauaggauccacagggagggggcauuucaauauauaauuacacuaaucauc

SEQ ID NO: 1067 - RNA, 100 nt, Artificial Sequence
  Description of Artificial Sequence Synthetic polynucleotide
gauauugugacauaucucugcacucaucacacaaaagagagacuucuucuauaggauccacagggagggggcauucuaauauauacguuuacuaaucauc

SEQ ID NO: 1068 - RNA, 100 nt, Artificial Sequence
  Description of Artificial Sequence Synthetic polynucleotide
gauauugugacauaucucugcacucaucacacaaaagaccucguucuucuauaggauccacagggagggggcaucuuaauauaugcacccacuaaucauc

SEQ ID NO: 1069 - RNA, 100 nt, Artificial Sequence
  Description of Artificial Sequence Synthetic polynucleotide
gauauugugacauaucucugcacucaucacacaaaagagauuucucuucuauaggauccacagggagggggcauuuuaauauauacgguuacuaaucauc

SEQ ID NO: 1070 - RNA, 100 nt, Artificial Sequence
  Description of Artificial Sequence Synthetic polynucleotide
gauauugugacauaucucugcacucaucacacaaaagucagguaucuucuauaggauccacagggagggggcauuuuaacauauaggaccacuaaucauc

SEQ ID NO: 1071 - RNA, 100 nt, Artificial Sequence
  Description of Artificial Sequence Synthetic polynucleotide
gauauugugacauaucucugcacucaucacacaaaagcgauucaucuucuauaggauccacagggagggggcauuucaauauauaagguuacuaaucauc

SEQ ID NO: 1072 - RNA, 100 nt, Artificial Sequence
  Description of Artificial Sequence Synthetic polynucleotide
gauauugugacauaucucugcacucaucacacaaaagcucgucaucuucuauaggauccacagggagggggcauucuaauauauggagauacuaaucauc

SEQ ID NO: 1073 - RNA, 100 nt, Artificial Sequence
  Description of Artificial Sequence Synthetic polynucleotide
gauauugugacauaucucugcacucaucacacaaaaguccgucaucuucuauaggauccacagggagggggcaucuuaauauaugagcccacuaaucauc

SEQ ID NO: 1074 - RNA, 100 nt, Artificial Sequence
  Description of Artificial Sequence Synthetic polynucleotide
gauauugugacauaucucugcacucaucacacaaaagccaucuaucuucuauaggauccacagggagggggcauuuuaauauauaccaucacuaaucauc

SEQ ID NO: 1075 - RNA, 100 nt, Artificial Sequence
  Description of Artificial Sequence Synthetic polynucleotide
gauauugugacauaucucugcacucaucacacaaaaucucguaaucuucuauaggauccacagggagggggcauuuuaacauaugcaauuacuaaucauc

SEQ ID NO: 1076 - RNA, 100 nt, Artificial Sequence
  Description of Artificial Sequence Synthetic polynucleotide
gauauugugacauaucucugcacucaucacacaaaaacccauaaucuucuauaggauccacagggagggggcauuucaauauauagauauacuaaucauc

SEQ ID NO: 1077 - RNA, 100 nt, Artificial Sequence
  Description of Artificial Sequence Synthetic polynucleotide
gauauugugacauaucucugcacucaucacacaaaacuacucaaucuucuauaggauccacagggagggggcauucuaauauauauacguacuaaucauc

SEQ ID NO: 1078 - RNA, 100 nt, Artificial Sequence
  Description of Artificial Sequence Synthetic polynucleotide
gauauugugacauaucucugcacucaucacacaaaaacguuuaaucuucuauaggauccacagggagggggcaucuuaauauauggagauacuaaucauc

SEQ ID NO: 1079 - RNA, 100 nt, Artificial Sequence
  Description of Artificial Sequence Synthetic polynucleotide
gauauugugacauaucucugcacucaucacacaaauucuagaaaucuucuauaggauccacagggagggggcauuucaauauauaaauuuacuaaucauc

SEQ ID NO: 1080 - RNA, 100 nt, Artificial Sequence
  Description of Artificial Sequence Synthetic polynucleotide
gauauugugacauaucucugcacucaucacacaaaucaaaaaaaucuucuauaggauccacagggagggggcauucuaauauauaaggccacuaaucauc

SEQ ID NO: 1081 - RNA, 100 nt, Artificial Sequence
  Description of Artificial Sequence Synthetic polynucleotide
gauauugugacauaucucugcacucaucacacaaacgcccaaaaucuucuauaggauccacagggagggggcaucuuaauauauaaguuuacuaaucauc

SEQ ID NO: 1082 - RNA, 100 nt, Artificial Sequence
  Description of Artificial Sequence Synthetic polynucleotide
gauauugugacauaucucugcacucaucacacaaacaucagaaaucuucuauaggauccacagggagggggcauuuuaauauaugaucacacuaaucauc

SEQ ID NO: 1083 - RNA, 100 nt, Artificial Sequence
  Description of Artificial Sequence Synthetic polynucleotide
gauauugugacauaucucugcacucaucacacaauccagucaaaucuucuauaggauccacagggagggggcauuuuaacauauagagucacuaaucauc

SEQ ID NO: 1084 - RNA, 100 nt, Artificial Sequence
  Description of Artificial Sequence Synthetic polynucleotide
gauauugugacauaucucugcacucaucacacaaucugaccaaaucuucuauaggauccacagggagggggcauuucaauauauaagucuacuaaucauc

SEQ ID NO: 1085 - RNA, 100 nt, Artificial Sequence
  Description of Artificial Sequence Synthetic polynucleotide
gauauugugacauaucucugcacucaucacacaauuacuucaaaucuucuauaggauccacagggagggggcauucuaauauauaugccuacuaaucauc

SEQ ID NO: 1086 - RNA, 100 nt, Artificial Sequence
  Description of Artificial Sequence Synthetic polynucleotide
gauauugugacauaucucugcacucaucacacaaccuaaccaaaucuucuauaggauccacagggagggggcaucuuaauauauaaacccacuaaucauc

SEQ ID NO: 1087 - RNA, 100 nt, Artificial Sequence
  Description of Artificial Sequence Synthetic polynucleotide
gauauugugacauaucucugcacucaucacacaaucuuaccaaaucuucuauaggauccacagggagggggcauuuuaauauaugcaauuacuaaucauc

SEQ ID NO: 1088 - RNA, 100 nt, Artificial Sequence
  Description of Artificial Sequence Synthetic polynucleotide
gauauugugacauaucucugcacucaucacacauugucagcaaaucuucuauaggauccacagggagggggcauuuuaacauauacgaauacuaaucauc

SEQ ID NO: 1089 - RNA, 100 nt, Artificial Sequence
  Description of Artificial Sequence Synthetic polynucleotide
gauauugugacauaucucugcacucaucacacacccaucgcaaaucuucuauaggauccacagggagggggcauuucaauauauggggucacuaaucauc

SEQ ID NO: 1090 - RNA, 100 nt, Artificial Sequence
  Description of Artificial Sequence Synthetic polynucleotide
gauauugugacauaucucugcacucaucacacauugucggcaaaucuucuauaggauccacagggagggggcauucuaauauauagaauuacuaaucauc

SEQ ID NO: 1091 - RNA, 100 nt, Artificial Sequence
  Description of Artificial Sequence Synthetic polynucleotide
gauauugugacauaucucugcacucaucacacauccacagcaaaucuucuauaggauccacagggagggggcaucuuaauauauggagccacuaaucauc

SEQ ID NO: 1092 - RNA, 100 nt, Artificial Sequence
  Description of Artificial Sequence Synthetic polynucleotide
gauauugugacauaucucugcacucaucacacacgcauggcaaaucuucuauaggauccacagggagggggcauuuuaauauauagaaauacuaaucauc

SEQ ID NO: 1093 - RNA, 100 nt, Artificial Sequence
  Description of Artificial Sequence Synthetic polynucleotide
gauauugugacauaucucugcacucaucacacucaucuugcaaaucuucuauaggauccacagggagggggcauuuuaacauauacacauacuaaucauc

SEQ ID NO: 1094 - RNA, 100 nt, Artificial Sequence
  Description of Artificial Sequence Synthetic polynucleotide
gauauugugacauaucucugcacucaucacacuuuaacugcaaaucuucuauaggauccacagggagggggcauuucaauauaugaccacacuaaucauc

SEQ ID NO: 1095 - RNA, 100 nt, Artificial Sequence
  Description of Artificial Sequence Synthetic polynucleotide
gauauugugacauaucucugcacucaucacaccucuauugcaaaucuucuauaggauccacagggagggggcauucuaauauaugauauuacuaaucauc

SEQ ID NO: 1096 - RNA, 100 nt, Artificial Sequence
  Description of Artificial Sequence Synthetic polynucleotide
gauauugugacauaucucugcacucaucacacugccacugcaaaucuucuauaggauccacagggagggggcaucuuaauauauggacacacuaaucauc

SEQ ID NO: 1097 - RNA, 100 nt, Artificial Sequence
  Description of Artificial Sequence Synthetic polynucleotide
gauauugugacauaucucugcacucaucacacugcuuuugcaaaucuucuauaggauccacagggagggggcauuuuaauauauaauauuacuaaucauc

SEQ ID NO: 1098 - RNA, 100 nt, Artificial Sequence
  Description of Artificial Sequence Synthetic polynucleotide
gauauugugacauaucucugcacucaucacagcacccaugcaaaucuucuauaggauccacagggagggggcauuuuaacauaugcgaccacuaaucauc

SEQ ID NO: 1099 - RNA, 100 nt, Artificial Sequence
  Description of Artificial Sequence Synthetic polynucleotide
gauauugugacauaucucugcacucaucacaaggucuaugcaaaucuucuauaggauccacagggagggggcauuucaauauaugcguccacuaaucauc

SEQ ID NO: 1100 - RNA, 100 nt, Artificial Sequence
  Description of Artificial Sequence Synthetic polynucleotide
gauauugugacauaucucugcacucaucacaguauacaugcaaaucuucuauaggauccacagggagggggcauucuaauauaugcguuuacuaaucauc

SEQ ID NO: 1101 - RNA, 100 nt, Artificial Sequence
  Description of Artificial Sequence Synthetic polynucleotide
gauauugugacauaucucugcacucaucacagucuaaaugcaaaucuucuauaggauccacagggagggggcaucuuaauauaugguuacacuaaucauc

SEQ ID NO: 1102 - RNA, 100 nt, Artificial Sequence
  Description of Artificial Sequence Synthetic polynucleotide
gauauugugacauaucucugcacucaucacaguugccaugcaaaucuucuauaggauccacagggagggggcauuuuaauauauauacgcacuaaucauc

SEQ ID NO: 1103 - RNA, 100 nt, Artificial Sequence
  Description of Artificial Sequence Synthetic polynucleotide
gauauugugacauaucucugcacucaucaccuacucgaugcaaaucuucuauaggauccacagggagggggcauuuuaacauaugcagcuacuaaucauc

SEQ ID NO: 1104 - RNA, 100 nt, Artificial Sequence
  Description of Artificial Sequence Synthetic polynucleotide
gauauugugacauaucucugcacucaucacugccccgaugcaaaucuucuauaggauccacagggagggggcauuucaauauauggguccacuaaucauc

SEQ ID NO: 1105 - RNA, 100 nt, Artificial Sequence
  Description of Artificial Sequence Synthetic polynucleotide
gauauugugacauaucucugcacucaucacugccuugaugcaaaucuucuauaggauccacagggagggggcaucuuaauauaugcgcauacuaaucauc

SEQ ID NO: 1106 - RNA, 100 nt, Artificial Sequence
  Description of Artificial Sequence Synthetic polynucleotide
gauauugugacauaucucugcacucaucaccugguugaugcaaaucuucuauaggauccacagggagggggcauuuuaauauaugggcacacuaaucauc

SEQ ID NO: 1107 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauaguaagaaacuaaucauc

SEQ ID NO: 1108 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauagcccauaacuaaucauc

SEQ ID NO: 1109 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauacgagauaacuaaucauc

SEQ ID NO: 1110 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauauaauguacaacuaaucauc

SEQ ID NO: 1111 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauaagagacaacuaaucauc

SEQ ID NO: 1112 - RNA, 100 nt, Artificial Sequence
  Description of Artificial Sequence Synthetic polynucleotide
gauauugugacauaucucugcacucaucacacaaaagaugcaaaucuucuauccaugacacagggagggggcauuuuaacauaguacgcaacuaaucauc

SEQ ID NO: 1113 - RNA, 100 nt, Artificial Sequence
  Description of Artificial Sequence Synthetic polynucleotide
gauauugugacauaucucugcacucaucacacaaaagaugcaaaucuucuauuuuugacacagggagggggcauuucaauauaagagccaacuaaucauc

SEQ ID NO: 1114 - RNA, 100 nt, Artificial Sequence
  Description of Artificial Sequence Synthetic polynucleotide
gauauugugacauaucucugcacucaucacacaaaagaugcaaaucuucuauccauggcacagggagggggcauucuaauauaagcaccaacuaaucauc

SEQ ID NO: 1115 - RNA, 100 nt, Artificial Sequence
  Description of Artificial Sequence Synthetic polynucleotide
gauauugugacauaucucugcacucaucacacaaaagaugcaaaucuucuauucuuggcacagggagggggcaucuuaauauaagcagcaacuaaucauc

SEQ ID NO: 1116 - RNA, 100 nt, Artificial Sequence
  Description of Artificial Sequence Synthetic polynucleotide
gauauugugacauaucucugcacucaucacacaaaagaugcaaaucuucuauccccagcacagggagggggcauuuuaauauacgaaacaacuaaucauc

SEQ ID NO: 1117 - RNA, 100 nt, Artificial Sequence
  Description of Artificial Sequence Synthetic polynucleotide
gauauugugacauaucucugcacucaucacacaaaagaugcaaaucuucuaccuuucccacagggagggggcauuuuaacauaguaaucaacuaaucauc

SEQ ID NO: 1118 - RNA, 100 nt, Artificial Sequence
  Description of Artificial Sequence Synthetic polynucleotide
gauauugugacauaucucugcacucaucacacaaaagaugcaaaucuucuacgaaacccacagggagggggcauuucaauauacagagaaacuaaucauc

SEQ ID NO: 1119 - RNA, 100 nt, Artificial Sequence
  Description of Artificial Sequence Synthetic polynucleotide
gauauugugacauaucucugcacucaucacacaaaagaugcaaaucuucuacucguaccacagggagggggcauucuaauauaaacggaaacuaaucauc

SEQ ID NO: 1120 - RNA, 100 nt, Artificial Sequence
  Description of Artificial Sequence Synthetic polynucleotide
gauauugugacauaucucugcacucaucacacaaaagaugcaaaucuucuaacaacaccacagggagggggcaucuuaauauaagcaguaacuaaucauc

SEQ ID NO: 1121 - RNA, 100 nt, Artificial Sequence
  Description of Artificial Sequence Synthetic polynucleotide
gauauugugacauaucucugcacucaucacacaaaagaugcaaaucuucuacucucgccacagggagggggcauuuuaauauagagguaaacuaaucauc

SEQ ID NO: 1122 - RNA, 100 nt, Artificial Sequence
  Description of Artificial Sequence Synthetic polynucleotide
gauauugugacauaucucugcacucaucacacaaaagaugcaaaucuucuuacauuuccacagggagggggcauuuuaacauagagguaaacuaaucauc

SEQ ID NO: 1123 - RNA, 100 nt, Artificial Sequence
  Description of Artificial Sequence Synthetic polynucleotide
gauauugugacauaucucugcacucaucacacaaaagaugcaaaucuucuuaauccuccacagggagggggcauuucaauauagcaggaaacuaaucauc

SEQ ID NO: 1124 - RNA, 100 nt, Artificial Sequence
  Description of Artificial Sequence Synthetic polynucleotide
gauauugugacauaucucugcacucaucacacaaaagaugcaaaucuucucagaauuccacagggagggggcauucuaauauaaacgucaacuaaucauc

SEQ ID NO: 1125 - RNA, 100 nt, Artificial Sequence
  Description of Artificial Sequence Synthetic polynucleotide
gauauugugacauaucucugcacucaucacacaaaagaugcaaaucuucuccguucuccacagggagggggcaucuuaauauaggcguuaacuaaucauc

SEQ ID NO: 1126 - RNA, 100 nt, Artificial Sequence
  Description of Artificial Sequence Synthetic polynucleotide
gauauugugacauaucucugcacucaucacacaaaagaugcaaaucuucucccucuuccacagggagggggcauuuuaauauaaggaguaacuaaucauc

SEQ ID NO: 1127 - RNA, 100 nt, Artificial Sequence
  Description of Artificial Sequence Synthetic polynucleotide
gauauugugacauaucucugcacucaucacacaaaagaugcaaaucuucagauucauccacagggagggggcauuuuaacauacggacaaacuaaucauc

SEQ ID NO: 1128 - RNA, 100 nt, Artificial Sequence
  Description of Artificial Sequence Synthetic polynucleotide
gauauugugacauaucucugcacucaucacacaaaagaugcaaaucuucacuccuauccacagggagggggcauuucaauauaaggaauaacuaaucauc

SEQ ID NO: 1129 - RNA, 100 nt, Artificial Sequence
  Description of Artificial Sequence Synthetic polynucleotide
gauauugugacauaucucugcacucaucacacaaaagaugcaaaucuucgucgauauccacagggagggggcauucuaauauaguacgcaacuaaucauc

SEQ ID NO: 1130 - RNA, 100 nt, Artificial Sequence
  Description of Artificial Sequence Synthetic polynucleotide
gauauugugacauaucucugcacucaucacacaaaagaugcaaaucuucacgucaauccacagggagggggcaucuuaauauaaggaguaacuaaucauc

SEQ ID NO: 1131 - RNA, 100 nt, Artificial Sequence
  Description of Artificial Sequence Synthetic polynucleotide
gauauugugacauaucucugcacucaucacacaaaagaugcaaaucuucccacucauccacagggagggggcauuuuaauauagacagcaacuaaucauc

SEQ ID NO: 1132 - RNA, 100 nt, Artificial Sequence
  Description of Artificial Sequence Synthetic polynucleotide
gauauugugacauaucucugcacucaucacacaaaagaugcaaaucuugcuccugauccacagggagggggcauuuuaacauagaaaaaaacuaaucauc

SEQ ID NO: 1133 - RNA, 100 nt, Artificial Sequence
  Description of Artificial Sequence Synthetic polynucleotide
gauauugugacauaucucugcacucaucacacaaaagaugcaaaucuuaaucgagauccacagggagggggcauuucaauauacggaccaacuaaucauc

SEQ ID NO: 1134 - RNA, 100 nt, Artificial Sequence
  Description of Artificial Sequence Synthetic polynucleotide
gauauugugacauaucucugcacucaucacacaaaagaugcaaaucuugcgguugauccacagggagggggcauucuaauauaggugcaaacuaaucauc

SEQ ID NO: 1135 - RNA, 100 nt, Artificial Sequence
  Description of Artificial Sequence Synthetic polynucleotide
gauauugugacauaucucugcacucaucacacaaaagaugcaaaucuuaaaauagauccacagggagggggcaucuuaauauaaagggcaacuaaucauc

SEQ ID NO: 1136 - RNA, 100 nt, Artificial Sequence
  Description of Artificial Sequence Synthetic polynucleotide
gauauugugacauaucucugcacucaucacacaaaagaugcaaaucuuggccucgauccacagggagggggcauuuuaauauaagaagaaacuaaucauc

SEQ ID NO: 1137 - RNA, 100 nt, Artificial Sequence
  Description of Artificial Sequence Synthetic polynucleotide
gauauugugacauaucucugcacucaucacacaaaagaugcaaaucuauaacuggauccacagggagggggcauuuuaacauaaccaccaacuaaucauc

SEQ ID NO: 1138 - RNA, 100 nt, Artificial Sequence
  Description of Artificial Sequence Synthetic polynucleotide
gauauugugacauaucucugcacucaucacacaaaagaugcaaaucucagcauggauccacagggagggggcauuucaauauacgccacaacuaaucauc

SEQ ID NO: 1139 - RNA, 100 nt, Artificial Sequence
  Description of Artificial Sequence Synthetic polynucleotide
gauauugugacauaucucugcacucaucacacaaaagaugcaaaucugacggcggauccacagggagggggcauucuaauauacacauaaacuaaucauc

SEQ ID NO: 1140 - RNA, 100 nt, Artificial Sequence
  Description of Artificial Sequence Synthetic polynucleotide
gauauugugacauaucucugcacucaucacacaaaagaugcaaaucuagcgauggauccacagggagggggcaucuuaauauaguagacaacuaaucauc

SEQ ID NO: 1141 - RNA, 100 nt, Artificial Sequence
  Description of Artificial Sequence Synthetic polynucleotide
gauauugugacauaucucugcacucaucacacaaaagaugcaaaucugaguccggauccacagggagggggcauuuuaauauaauggguaacuaaucauc

SEQ ID NO: 1142 - RNA, 100 nt, Artificial Sequence
  Description of Artificial Sequence Synthetic polynucleotide
gauauugugacauaucucugcacucaucacacaaaagaugcaaaucgcgacgaggauccacagggagggggcauuuuaacauaggagucaacuaaucauc

SEQ ID NO: 1143 - RNA, 100 nt, Artificial Sequence
  Description of Artificial Sequence Synthetic polynucleotide
gauauugugacauaucucugcacucaucacacaaaagaugcaaaucccagggaggauccacagggagggggcauuucaauauaagcaccaacuaaucauc

SEQ ID NO: 1144 - RNA, 100 nt, Artificial Sequence
  Description of Artificial Sequence Synthetic polynucleotide
gauauugugacauaucucugcacucaucacacaaaagaugcaaaucaacaagaggauccacagggagggggcauucuaauauaaugaccaacuaaucauc

SEQ ID NO: 1145 - RNA, 100 nt, Artificial Sequence
  Description of Artificial Sequence Synthetic polynucleotide
gauauugugacauaucucugcacucaucacacaaaagaugcaaaucguaccgaggauccacagggagggggcaucuuaauauagaguauaacuaaucauc

SEQ ID NO: 1146 - RNA, 100 nt, Artificial Sequence
  Description of Artificial Sequence Synthetic polynucleotide
gauauugugacauaucucugcacucaucacacaaaagaugcaaaucggucagaggauccacagggagggggcauuuuaauauaggccgcaacuaaucauc

SEQ ID NO: 1147 - RNA, 100 nt, Artificial Sequence
  Description of Artificial Sequence Synthetic polynucleotide
gauauugugacauaucucugcacucaucacacaaaagaugcaaaugcauccuaggauccacagggagggggcauuuuaacauacgggucaacuaaucauc

SEQ ID NO: 1148 - RNA, 100 nt, Artificial Sequence
  Description of Artificial Sequence Synthetic polynucleotide
gauauugugacauaucucugcacucaucacacaaaagaugcaaauagauucuaggauccacagggagggggcauuucaauauaaaaaguaacuaaucauc

SEQ ID NO: 1149 - RNA, 100 nt, Artificial Sequence
  Description of Artificial Sequence Synthetic polynucleotide
gauauugugacauaucucugcacucaucacacaaaagaugcaaaugaggccuaggauccacagggagggggcauucuaauauacggaauaacuaaucauc

SEQ ID NO: 1150 - RNA, 100 nt, Artificial Sequence
  Description of Artificial Sequence Synthetic polynucleotide
gauauugugacauaucucugcacucaucacacaaaagaugcaaauaaaaguuaggauccacagggagggggcaucuuaauauagggagcaacuaaucauc

SEQ ID NO: 1151 - RNA, 100 nt, Artificial Sequence
  Description of Artificial Sequence Synthetic polynucleotide
gauauugugacauaucucugcacucaucacacaaaagaugcaaaugacaguuaggauccacagggagggggcauuuuaauauagacgucaacuaaucauc

SEQ ID NO: 1152 - RNA, 100 nt, Artificial Sequence
  Description of Artificial Sequence Synthetic polynucleotide
gauauugugacauaucucugcacucaucacacaaaagaugcaaacaacggauaggauccacagggagggggcauuuuaacauaaugggaaacuaaucauc

SEQ ID NO: 1153 - RNA, 100 nt, Artificial Sequence
  Description of Artificial Sequence Synthetic polynucleotide
gauauugugacauaucucugcacucaucacacaaaagaugcaaaaaccgaauaggauccacagggagggggcauuucaauauaccagguaacuaaucauc

SEQ ID NO: 1154 - RNA, 100 nt, Artificial Sequence
  Description of Artificial Sequence Synthetic polynucleotide
gauauugugacauaucucugcacucaucacacaaaagaugcaaaaacgacauaggauccacagggagggggcauucuaauauagaaaccaacuaaucauc

SEQ ID NO: 1155 - RNA, 100 nt, Artificial Sequence
  Description of Artificial Sequence Synthetic polynucleotide
gauauugugacauaucucugcacucaucacacaaaagaugcaaaaugcacauaggauccacagggagggggcaucuuaauauaauauaaaacuaaucauc

SEQ ID NO: 1156 - RNA, 100 nt, Artificial Sequence
  Description of Artificial Sequence Synthetic polynucleotide
gauauugugacauaucucugcacucaucacacaaaagaugcaaaggccggauaggauccacagggagggggcauuuuaauauaagcagaaacuaaucauc

SEQ ID NO: 1157 - RNA, 100 nt, Artificial Sequence
  Description of Artificial Sequence Synthetic polynucleotide
gauauugugacauaucucugcacucaucacacaaaagaugcaacgaaaguauaggauccacagggagggggcauuuuaacauagccgcaaacuaaucauc

SEQ ID NO: 1158 - RNA, 100 nt, Artificial Sequence
  Description of Artificial Sequence Synthetic polynucleotide
gauauugugacauaucucugcacucaucacacaaaagaugcaauaaugguauaggauccacagggagggggcauuucaauauagcacauaacuaaucauc

SEQ ID NO: 1159 - RNA, 100 nt, Artificial Sequence
  Description of Artificial Sequence Synthetic polynucleotide
gauauugugacauaucucugcacucaucacacaaaagaugcaacacauauauaggauccacagggagggggcauucuaauauaagcaauaacuaaucauc

SEQ ID NO: 1160 - RNA, 100 nt, Artificial Sequence
  Description of Artificial Sequence Synthetic polynucleotide
gauauugugacauaucucugcacucaucacacaaaagaugcaauugcuauauaggauccacagggagggggcaucuuaauauagaagacaacuaaucauc

SEQ ID NO: 1161 - RNA, 100 nt, Artificial Sequence
  Description of Artificial Sequence Synthetic polynucleotide
gauauugugacauaucucugcacucaucacacaaaagaugcaacaauuauauaggauccacagggagggggcauuuuaauauaggcgccaacuaaucauc

SEQ ID NO: 1162 - RNA, 100 nt, Artificial Sequence
  Description of Artificial Sequence Synthetic polynucleotide
gauauugugacauaucucugcacucaucacacaaaagaugcaucgagccuauaggauccacagggagggggcauuuuaacauaaagguuaacuaaucauc

SEQ ID NO: 1163 - RNA, 100 nt, Artificial Sequence
  Description of Artificial Sequence Synthetic polynucleotide
gauauugugacauaucucugcacucaucacacaaaagaugcacccagccuauaggauccacagggagggggcauuucaauauaaugagaaacuaaucauc

SEQ ID NO: 1164 - RNA, 100 nt, Artificial Sequence
  Description of Artificial Sequence Synthetic polynucleotide
gauauugugacauaucucugcacucaucacacaaaagaugcacucacacuauaggauccacagggagggggcauucuaauauaggugcaaacuaaucauc

SEQ ID NO: 1165 - RNA, 100 nt, Artificial Sequence
  Description of Artificial Sequence Synthetic polynucleotide
gauauugugacauaucucugcacucaucacacaaaagaugcacugggccuauaggauccacagggagggggcaucuuaauauaggcaccaacuaaucauc

SEQ ID NO: 1166 - RNA, 100 nt, Artificial Sequence
  Description of Artificial Sequence Synthetic polynucleotide
gauauugugacauaucucugcacucaucacacaaaagaugcacccggacuauaggauccacagggagggggcauuuuaauauaauacacaacuaaucauc

SEQ ID NO: 1167 - RNA, 100 nt, Artificial Sequence
  Description of Artificial Sequence Synthetic polynucleotide
gauauugugacauaucucugcacucaucacacaaaagaugcucaggcucuauaggauccacagggagggggcauuuuaacauaaaagccaacuaaucauc

SEQ ID NO: 1168 - RNA, 100 nt, Artificial Sequence
  Description of Artificial Sequence Synthetic polynucleotide
gauauugugacauaucucugcacucaucacacaaaagaugccgcggaucuauaggauccacagggagggggcauuucaauauaguagucaacuaaucauc

SEQ ID NO: 1169 - RNA, 100 nt, Artificial Sequence
  Description of Artificial Sequence Synthetic polynucleotide
gauauugugacauaucucugcacucaucacacaaaagaugccuggcgucuauaggauccacagggagggggcauucuaauauacgaaauaacuaaucauc

SEQ ID NO: 1170 - RNA, 100 nt, Artificial Sequence
  Description of Artificial Sequence Synthetic polynucleotide
gauauugugacauaucucugcacucaucacacaaaagaugccugcggucuauaggauccacagggagggggcaucuuaauauaagggaaaacuaaucauc

SEQ ID NO: 1171 - RNA, 100 nt, Artificial Sequence
  Description of Artificial Sequence Synthetic polynucleotide
gauauugugacauaucucugcacucaucacacaaaagaugccccgaaucuauaggauccacagggagggggcauuuuaauauacccaaaaacuaaucauc

SEQ ID NO: 1172 - RNA, 100 nt, Artificial Sequence
  Description of Artificial Sequence Synthetic polynucleotide
gauauugugacauaucucugcacucaucacacaaaagaugacgccguucuauaggauccacagggagggggcauuuuaacauagaggauaacuaaucauc

SEQ ID NO: 1173 - RNA, 100 nt, Artificial Sequence
  Description of Artificial Sequence Synthetic polynucleotide
gauauugugacauaucucugcacucaucacacaaaagaugaauacguucuauaggauccacagggagggggcauuucaauauaagcguuaacuaaucauc

SEQ ID NO: 1174 - RNA, 100 nt, Artificial Sequence
  Description of Artificial Sequence Synthetic polynucleotide
gauauugugacauaucucugcacucaucacacaaaagaugguucaguucuauaggauccacagggagggggcauucuaauauaaugggaaacuaaucauc

SEQ ID NO: 1175 - RNA, 100 nt, Artificial Sequence
  Description of Artificial Sequence Synthetic polynucleotide
gauauugugacauaucucugcacucaucacacaaaagaugguaccauucuauaggauccacagggagggggcaucuuaauauaggagccaacuaaucauc

SEQ ID NO: 1176 - RNA, 100 nt, Artificial Sequence
  Description of Artificial Sequence Synthetic polynucleotide
gauauugugacauaucucugcacucaucacacaaaagaugguugaguucuauaggauccacagggagggggcauuuuaauauagacggcaacuaaucauc

SEQ ID NO: 1177 - RNA, 100 nt, Artificial Sequence
  Description of Artificial Sequence Synthetic polynucleotide
gauauugugacauaucucugcacucaucacacaaaagauaacucacuucuauaggauccacagggagggggcauuuuaacauagaguauaacuaaucauc

SEQ ID NO: 1178 - RNA, 100 nt, Artificial Sequence
  Description of Artificial Sequence Synthetic polynucleotide
gauauugugacauaucucugcacucaucacacaaaagauugaccacuucuauaggauccacagggagggggcauuucaauauaaaccgcaacuaaucauc

SEQ ID NO: 1179 - RNA, 100 nt, Artificial Sequence
  Description of Artificial Sequence Synthetic polynucleotide
gauauugugacauaucucugcacucaucacacaaaagaucuuacgcuucuauaggauccacagggagggggcauucuaauauacgcacaaacuaaucauc

SEQ ID NO: 1180 - RNA, 100 nt, Artificial Sequence
  Description of Artificial Sequence Synthetic polynucleotide
gauauugugacauaucucugcacucaucacacaaaagauuuaccacuucuauaggauccacagggagggggcaucuuaauauacggacaaacuaaucauc

SEQ ID NO: 1181 - RNA, 100 nt, Artificial Sequence
  Description of Artificial Sequence Synthetic polynucleotide
gauauugugacauaucucugcacucaucacacaaaagaucuccaccuucuauaggauccacagggagggggcauuuuaauauaaacaccaacuaaucauc

SEQ ID NO: 1182 - RNA, 100 nt, Artificial Sequence
  Description of Artificial Sequence Synthetic polynucleotide
gauauugugacauaucucugcacucaucacacaaaagagaggcuucuucuauaggauccacagggagggggcauuuuaacauacaggacaacuaaucauc

SEQ ID NO: 1183 - RNA, 100 nt, Artificial Sequence
  Description of Artificial Sequence Synthetic polynucleotide
gauauugugacauaucucugcacucaucacacaaaagagaauccucuucuauaggauccacagggagggggcauuucaauauaauggccaacuaaucauc

SEQ ID NO: 1184 - RNA, 100 nt, Artificial Sequence
  Description of Artificial Sequence Synthetic polynucleotide
gauauugugacauaucucugcacucaucacacaaaagaaaguccucuucuauaggauccacagggagggggcauucuaauauagcagcaaacuaaucauc

SEQ ID NO: 1185 - RNA, 100 nt, Artificial Sequence
  Description of Artificial Sequence Synthetic polynucleotide
gauauugugacauaucucugcacucaucacacaaaagagaccucucuucuauaggauccacagggagggggcaucuuaauauaaaggucaacuaaucauc

SEQ ID NO: 1186 - RNA, 100 nt, Artificial Sequence
  Description of Artificial Sequence Synthetic polynucleotide
gauauugugacauaucucugcacucaucacacaaaagcaauacaucuucuauaggauccacagggagggggcauuuuaacauagcggucaacuaaucauc

SEQ ID NO: 1187 - RNA, 100 nt, Artificial Sequence
  Description of Artificial Sequence Synthetic polynucleotide
gauauugugacauaucucugcacucaucacacaaaagcauaucaucuucuauaggauccacagggagggggcauuucaauauaaacagaaacuaaucauc

SEQ ID NO: 1188 - RNA, 100 nt, Artificial Sequence
  Description of Artificial Sequence Synthetic polynucleotide
gauauugugacauaucucugcacucaucacacaaaagcuagauaucuucuauaggauccacagggagggggcauucuaauauacacccaaacuaaucauc

SEQ ID NO: 1189 - RNA, 100 nt, Artificial Sequence
  Description of Artificial Sequence Synthetic polynucleotide
gauauugugacauaucucugcacucaucacacaaaagucaguuaucuucuauaggauccacagggagggggcaucuuaauauaauagauaacuaaucauc

SEQ ID NO: 1190 - RNA, 100 nt, Artificial Sequence
  Description of Artificial Sequence Synthetic polynucleotide
gauauugugacauaucucugcacucaucacacaaaagcuagguaucuucuauaggauccacagggagggggcauuuuaauauacgagccaacuaaucauc

SEQ ID NO: 1191 - RNA, 100 nt, Artificial Sequence
  Description of Artificial Sequence Synthetic polynucleotide
gauauugugacauaucucugcacucaucacacaaaauccaauaaucuucuauaggauccacagggagggggcauuuuaacauaguauucaacuaaucauc

SEQ ID NO: 1192 - RNA, 100 nt, Artificial Sequence
  Description of Artificial Sequence Synthetic polynucleotide
gauauugugacauaucucugcacucaucacacaaaacggaccaaucuucuauaggauccacagggagggggcauuucaauauaccaaacaacuaaucauc

SEQ ID NO: 1193 - RNA, 100 nt, Artificial Sequence
  Description of Artificial Sequence Synthetic polynucleotide
gauauugugacauaucucugcacucaucacacaaaacccccuaaucuucuauaggauccacagggagggggcauucuaauauacgggcaaacuaaucauc

SEQ ID NO: 1194 - RNA, 100 nt, Artificial Sequence
  Description of Artificial Sequence Synthetic polynucleotide
gauauugugacauaucucugcacucaucacacaaaaccguuuaaucuucuauaggauccacagggagggggcaucuuaauauacgguauaacuaaucauc

SEQ ID NO: 1195 - RNA, 100 nt, Artificial Sequence
  Description of Artificial Sequence Synthetic polynucleotide
gauauugugacauaucucugcacucaucacacaaaacaaccuaaucuucuauaggauccacagggagggggcauuuuaauauaauagacaacuaaucauc

SEQ ID NO: 1196 - RNA, 100 nt, Artificial Sequence
  Description of Artificial Sequence Synthetic polynucleotide
gauauugugacauaucucugcacucaucacacaaacccccgaaaucuucuauaggauccacagggagggggcauuuuaacauaagcacaaacuaaucauc

SEQ ID NO: 1197 - RNA, 100 nt, Artificial Sequence
  Description of Artificial Sequence Synthetic polynucleotide
gauauugugacauaucucugcacucaucacacaaaccgcagaaaucuucuauaggauccacagggagggggcauuucaauauaaaaguaaacuaaucauc

SEQ ID NO: 1198 - RNA, 100 nt, Artificial Sequence
  Description of Artificial Sequence Synthetic polynucleotide
gauauugugacauaucucugcacucaucacacaaacccaaaaaaucuucuauaggauccacagggagggggcauucuaauauagccgcaaacuaaucauc

SEQ ID NO: 1199 - RNA, 100 nt, Artificial Sequence
  Description of Artificial Sequence Synthetic polynucleotide
gauauugugacauaucucugcacucaucacacaaaccacaaaaaucuucuauaggauccacagggagggggcaucuuaauauagaacgcaacuaaucauc

SEQ ID NO: 1200 - RNA, 100 nt, Artificial Sequence
  Description of Artificial Sequence Synthetic polynucleotide
gauauugugacauaucucugcacucaucacacaaauuugagaaaucuucuauaggauccacagggagggggcauuuuaauauagcaaccaacuaaucauc

SEQ ID NO: 1201 - RNA, 100 nt, Artificial Sequence
  Description of Artificial Sequence Synthetic polynucleotide
gauauugugacauaucucugcacucaucacacaaccuaaucaaaucuucuauaggauccacagggagggggcauuuuaacauagaaguuaacuaaucauc

SEQ ID NO: 1202 - RNA, 100 nt, Artificial Sequence
  Description of Artificial Sequence Synthetic polynucleotide
gauauugugacauaucucugcacucaucacacaacacgcucaaaucuucuauaggauccacagggagggggcauuucaauauaagagaaaacuaaucauc

SEQ ID NO: 1203 - RNA, 100 nt, Artificial Sequence
  Description of Artificial Sequence Synthetic polynucleotide
gauauugugacauaucucugcacucaucacacaacacgcccaaaucuucuauaggauccacagggagggggcauucuaauauaccagacaacuaaucauc

SEQ ID NO: 1204 - RNA, 100 nt, Artificial Sequence
  Description of Artificial Sequence Synthetic polynucleotide
gauauugugacauaucucugcacucaucacacaacauagccaaaucuucuauaggauccacagggagggggcaucuuaauauacgguaaaacuaaucauc

SEQ ID NO: 1205 - RNA, 100 nt, Artificial Sequence
  Description of Artificial Sequence Synthetic polynucleotide
gauauugugacauaucucugcacucaucacacaacuacuccaaaucuucuauaggauccacagggagggggcauuuuaauauaguggccaacuaaucauc

SEQ ID NO: 1206 - RNA, 100 nt, Artificial Sequence
  Description of Artificial Sequence Synthetic polynucleotide
gauauugugacauaucucugcacucaucacacaccauucgcaaaucuucuauaggauccacagggagggggcauuuuaacauagggaguaacuaaucauc

SEQ ID NO: 1207 - RNA, 100 nt, Artificial Sequence
  Description of Artificial Sequence Synthetic polynucleotide
gauauugugacauaucucugcacucaucacacauguacggcaaaucuucuauaggauccacagggagggggcauuucaauauacagggcaacuaaucauc

SEQ ID NO: 1208 - RNA, 100 nt, Artificial Sequence
  Description of Artificial Sequence Synthetic polynucleotide
gauauugugacauaucucugcacucaucacacauuaauggcaaaucuucuauaggauccacagggagggggcauucuaauauagagaccaacuaaucauc

SEQ ID NO: 1209 - RNA, 100 nt, Artificial Sequence
  Description of Artificial Sequence Synthetic polynucleotide
gauauugugacauaucucugcacucaucacacaccauaggcaaaucuucuauaggauccacagggagggggcaucuuaauauaggagacaacuaaucauc

SEQ ID NO: 1210 - RNA, 100 nt, Artificial Sequence
  Description of Artificial Sequence Synthetic polynucleotide
gauauugugacauaucucugcacucaucacacacugccagcaaaucuucuauaggauccacagggagggggcauuuuaauauacaacgcaacuaaucauc

SEQ ID NO: 1211 - RNA, 100 nt, Artificial Sequence
  Description of Artificial Sequence Synthetic polynucleotide
gauauugugacauaucucugcacucaucacacccccucugcaaaucuucuauaggauccacagggagggggcauuuuaacauaacaggcaacuaaucauc

SEQ ID NO: 1212 - RNA, 100 nt, Artificial Sequence
  Description of Artificial Sequence Synthetic polynucleotide
gauauugugacauaucucugcacucaucacacucguccugcaaaucuucuauaggauccacagggagggggcauuucaauauagacauuaacuaaucauc

SEQ ID NO: 1213 - RNA, 100 nt, Artificial Sequence
  Description of Artificial Sequence Synthetic polynucleotide
gauauugugacauaucucugcacucaucacaccuacacugcaaaucuucuauaggauccacagggagggggcauucuaauauagggaguaacuaaucauc

SEQ ID NO: 1214 - RNA, 100 nt, Artificial Sequence
  Description of Artificial Sequence Synthetic polynucleotide
gauauugugacauaucucugcacucaucacacucuuacugcaaaucuucuauaggauccacagggagggggcaucuuaauauagccacaaacuaaucauc

SEQ ID NO: 1215 - RNA, 100 nt, Artificial Sequence
  Description of Artificial Sequence Synthetic polynucleotide
gauauugugacauaucucugcacucaucacaccucucuugcaaaucuucuauaggauccacagggagggggcauuuuaauauaguagcaaacuaaucauc

SEQ ID NO: 1216 - RNA, 100 nt, Artificial Sequence
  Description of Artificial Sequence Synthetic polynucleotide
gauauugugacauaucucugcacucaucacaguaaccaugcaaaucuucuauaggauccacagggagggggcauuuuaacauaguagguaacuaaucauc

SEQ ID NO: 1217 - RNA, 100 nt, Artificial Sequence
  Description of Artificial Sequence Synthetic polynucleotide
gauauugugacauaucucugcacucaucacaaacuuaaugcaaaucuucuauaggauccacagggagggggcauuucaauauaguggaaaacuaaucauc

SEQ ID NO: 1218 - RNA, 100 nt, Artificial Sequence
  Description of Artificial Sequence Synthetic polynucleotide
gauauugugacauaucucugcacucaucacaacucauaugcaaaucuucuauaggauccacagggagggggcauucuaauauaaggaccaacuaaucauc

SEQ ID NO: 1219 - RNA, 100 nt, Artificial Sequence
  Description of Artificial Sequence Synthetic polynucleotide
gauauugugacauaucucugcacucaucacagcuaucaugcaaaucuucuauaggauccacagggagggggcaucuuaauauagaaggaaacuaaucauc

SEQ ID NO: 1220 - RNA, 100 nt, Artificial Sequence
  Description of Artificial Sequence Synthetic polynucleotide
gauauugugacauaucucugcacucaucacaagugcuaugcaaaucuucuauaggauccacagggagggggcauuuuaauauagggagcaacuaaucauc

SEQ ID NO: 1221 - RNA, 100 nt, Artificial Sequence
  Description of Artificial Sequence Synthetic polynucleotide
gauauugugacauaucucugcacucaucacuucuucgaugcaaaucuucuauaggauccacagggagggggcauuuuaacauaaacacaaacuaaucauc

SEQ ID NO: 1222 - RNA, 100 nt, Artificial Sequence
  Description of Artificial Sequence Synthetic polynucleotide
gauauugugacauaucucugcacucaucacuuggccgaugcaaaucuucuauaggauccacagggagggggcauuucaauauagcccauaacuaaucauc

SEQ ID NO: 1223 - RNA, 100 nt, Artificial Sequence
  Description of Artificial Sequence Synthetic polynucleotide
gauauugugacauaucucugcacucaucacugguccgaugcaaaucuucuauaggauccacagggagggggcauucuaauauagcacauaacuaaucauc

SEQ ID NO: 1224 - RNA, 100 nt, Artificial Sequence
  Description of Artificial Sequence Synthetic polynucleotide
gauauugugacauaucucugcacucaucaccuuuaugaugcaaaucuucuauaggauccacagggagggggcaucuuaauauacagauaaacuaaucauc

SEQ ID NO: 1225 - RNA, 100 nt, Artificial Sequence
  Description of Artificial Sequence Synthetic polynucleotide
gauauugugacauaucucugcacucaucaccuuguugaugcaaaucuucuauaggauccacagggagggggcauuuuaauauacccaaaaacuaaucauc

SEQ ID NO: 1226 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacauuggcgcgaacuaaucauc

SEQ ID NO: 1227 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauauuguaccgaacuaaucauc

SEQ ID NO: 1228 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauauucagaggaacuaaucauc

SEQ ID NO: 1229 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauaucggcaagaacuaaucauc

SEQ ID NO: 1230 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauauuggcccgaacuaaucauc

SEQ ID NO: 1231 - RNA, 100 nt, Artificial Sequence
  Description of Artificial Sequence Synthetic polynucleotide
gauauugugacauaucucugcacucaucacacaaaagaugcaaaucuucuaagccuaccacagggagggggcauuuuaacauuggggggaacuaaucauc

SEQ ID NO: 1232 - RNA, 100 nt, Artificial Sequence
  Description of Artificial Sequence Synthetic polynucleotide
gauauugugacauaucucugcacucaucacacaaaagaugcaaaucuucuaacucacccacagggagggggcauuucaauaucagaaggaacuaaucauc

SEQ ID NO: 1233 - RNA, 100 nt, Artificial Sequence
  Description of Artificial Sequence Synthetic polynucleotide
gauauugugacauaucucugcacucaucacacaaaagaugcaaaucuucuacccacaccacagggagggggcauucuaauauucauacgaacuaaucauc

SEQ ID NO: 1234 - RNA, 100 nt, Artificial Sequence
  Description of Artificial Sequence Synthetic polynucleotide
gauauugugacauaucucugcacucaucacacaaaagaugcaaaucuucuagccgcgccacagggagggggcaucuuaauauucacccgaacuaaucauc

SEQ ID NO: 1235 - RNA, 100 nt, Artificial Sequence
  Description of Artificial Sequence Synthetic polynucleotide
gauauugugacauaucucugcacucaucacacaaaagaugcaaaucuucuagaaaugccacagggagggggcauuuuaauauuguggcgaacuaaucauc

SEQ ID NO: 1236 - RNA, 100 nt, Artificial Sequence
  Description of Artificial Sequence Synthetic polynucleotide
gauauugugacauaucucugcacucaucacacaaaagaugcaaaucuucuccuucuuccacagggagggggcauuuuaacauuagaaagaacuaaucauc

SEQ ID NO: 1237 - RNA, 100 nt, Artificial Sequence
  Description of Artificial Sequence Synthetic polynucleotide
gauauugugacauaucucugcacucaucacacaaaagaugcaaaucuucuuauaccuccacagggagggggcauuucaauauuaccaggaacuaaucauc

SEQ ID NO: 1238 - RNA, 100 nt, Artificial Sequence
  Description of Artificial Sequence Synthetic polynucleotide
gauauugugacauaucucugcacucaucacacaaaagaugcaaaucuucuuuuauuuccacagggagggggcauucuaauaucauggagaacuaaucauc

SEQ ID NO: 1239 - RNA, 100 nt, Artificial Sequence
  Description of Artificial Sequence Synthetic polynucleotide
gauauugugacauaucucugcacucaucacacaaaagaugcaaaucuucuugcaauuccacagggagggggcaucuuaauauuugggggaacuaaucauc

SEQ ID NO: 1240 - RNA, 100 nt, Artificial Sequence
  Description of Artificial Sequence Synthetic polynucleotide
gauauugugacauaucucugcacucaucacacaaaagaugcaaaucuuccgagucauccacagggagggggcauuuuaacauugaggggaacuaaucauc

SEQ ID NO: 1241 - RNA, 100 nt, Artificial Sequence
  Description of Artificial Sequence Synthetic polynucleotide
gauauugugacauaucucugcacucaucacacaaaagaugcaaaucuuccucguuauccacagggagggggcauuucaauaucggaaggaacuaaucauc

SEQ ID NO: 1242 - RNA, 100 nt, Artificial Sequence
  Description of Artificial Sequence Synthetic polynucleotide
gauauugugacauaucucugcacucaucacacaaaagaugcaaaucuucacaaauauccacagggagggggcauucuaauauuuggaagaacuaaucauc

SEQ ID NO: 1243 - RNA, 100 nt, Artificial Sequence
  Description of Artificial Sequence Synthetic polynucleotide
gauauugugacauaucucugcacucaucacacaaaagaugcaaaucuucagaauaauccacagggagggggcaucuuaauauccuacggaacuaaucauc

SEQ ID NO: 1244 - RNA, 100 nt, Artificial Sequence
  Description of Artificial Sequence Synthetic polynucleotide
gauauugugacauaucucugcacucaucacacaaaagaugcaaaucuucacaacuauccacagggagggggcauuuuaauauuaaguagaacuaaucauc

SEQ ID NO: 1245 - RNA, 100 nt, Artificial Sequence
  Description of Artificial Sequence Synthetic polynucleotide
gauauugugacauaucucugcacucaucacacaaaagaugcaaaucuuaaccacgauccacagggagggggcauuucaauaucauauagaacuaaucauc

SEQ ID NO: 1246 - RNA, 100 nt, Artificial Sequence
  Description of Artificial Sequence Synthetic polynucleotide
gauauugugacauaucucugcacucaucacacaaaagaugcaaaucuuaaggcagauccacagggagggggcauucuaauauuaguacgaacuaaucauc

SEQ ID NO: 1247 - RNA, 100 nt, Artificial Sequence
  Description of Artificial Sequence Synthetic polynucleotide
gauauugugacauaucucugcacucaucacacaaaagaugcaaaucuugcccgagauccacagggagggggcaucuuaauauuaaagggaacuaaucauc

SEQ ID NO: 1248 - RNA, 100 nt, Artificial Sequence
  Description of Artificial Sequence Synthetic polynucleotide
gauauugugacauaucucugcacucaucacacaaaagaugcaaaucuuaucuuagauccacagggagggggcauuuuaauauuccauagaacuaaucauc

SEQ ID NO: 1249 - RNA, 100 nt, Artificial Sequence
  Description of Artificial Sequence Synthetic polynucleotide
gauauugugacauaucucugcacucaucacacaaaagaugcaaaucuccccccggauccacagggagggggcauuuuaacauuaaauggaacuaaucauc

SEQ ID NO: 1250 - RNA, 100 nt, Artificial Sequence
  Description of Artificial Sequence Synthetic polynucleotide
gauauugugacauaucucugcacucaucacacaaaagaugcaaaucugggacuggauccacagggagggggcauuucaauauuugggcgaacuaaucauc

SEQ ID NO: 1251 - RNA, 100 nt, Artificial Sequence
  Description of Artificial Sequence Synthetic polynucleotide
gauauugugacauaucucugcacucaucacacaaaagaugcaaaucuguauuuggauccacagggagggggcauucuaauauuuaagagaacuaaucauc

SEQ ID NO: 1252 - RNA, 100 nt, Artificial Sequence
  Description of Artificial Sequence Synthetic polynucleotide
gauauugugacauaucucugcacucaucacacaaaagaugcaaaucuccacccggauccacagggagggggcaucuuaauauuugguagaacuaaucauc

SEQ ID NO: 1253 - RNA, 100 nt, Artificial Sequence
  Description of Artificial Sequence Synthetic polynucleotide
gauauugugacauaucucugcacucaucacacaaaagaugcaaaucuagcaccggauccacagggagggggcauuuuaauauccgcaagaacuaaucauc

SEQ ID NO: 1254 - RNA, 100 nt, Artificial Sequence
  Description of Artificial Sequence Synthetic polynucleotide
gauauugugacauaucucugcacucaucacacaaaagaugcaaaucauaccgaggauccacagggagggggcauuuuaacauuccagggaacuaaucauc

SEQ ID NO: 1255 - RNA, 100 nt, Artificial Sequence
  Description of Artificial Sequence Synthetic polynucleotide
gauauugugacauaucucugcacucaucacacaaaagaugcaaaucgaaucaaggauccacagggagggggcauuucaauauuagcgcgaacuaaucauc

SEQ ID NO: 1256 - RNA, 100 nt, Artificial Sequence
  Description of Artificial Sequence Synthetic polynucleotide
gauauugugacauaucucugcacucaucacacaaaagaugcaaaucggucacaggauccacagggagggggcauucuaauauccaccagaacuaaucauc

SEQ ID NO: 1257 - RNA, 100 nt, Artificial Sequence
  Description of Artificial Sequence Synthetic polynucleotide
gauauugugacauaucucugcacucaucacacaaaagaugcaaauccaucagaggauccacagggagggggcaucuuaauauuuuagggaacuaaucauc

SEQ ID NO: 1258 - RNA, 100 nt, Artificial Sequence
  Description of Artificial Sequence Synthetic polynucleotide
gauauugugacauaucucugcacucaucacacaaaagaugcaaauccgacggaggauccacagggagggggcauuuuaauauucaggggaacuaaucauc

SEQ ID NO: 1259 - RNA, 100 nt, Artificial Sequence
  Description of Artificial Sequence Synthetic polynucleotide
gauauugugacauaucucugcacucaucacacaaaagaugcaaauauuagcuaggauccacagggagggggcauuuuaacauuaacaagaacuaaucauc

SEQ ID NO: 1260 - RNA, 100 nt, Artificial Sequence
  Description of Artificial Sequence Synthetic polynucleotide
gauauugugacauaucucugcacucaucacacaaaagaugcaaauaagaguuaggauccacagggagggggcauuucaauauccgaacgaacuaaucauc

SEQ ID NO: 1261 - RNA, 100 nt, Artificial Sequence
  Description of Artificial Sequence Synthetic polynucleotide
gauauugugacauaucucugcacucaucacacaaaagaugcaaaugccaccuaggauccacagggagggggcauucuaauauuagggagaacuaaucauc

SEQ ID NO: 1262 - RNA, 100 nt, Artificial Sequence
  Description of Artificial Sequence Synthetic polynucleotide
gauauugugacauaucucugcacucaucacacaaaagaugcaaauaauacuuaggauccacagggagggggcaucuuaauauuagcgagaacuaaucauc

SEQ ID NO: 1263 - RNA, 100 nt, Artificial Sequence
  Description of Artificial Sequence Synthetic polynucleotide
gauauugugacauaucucugcacucaucacacaaaagaugcaaaugacguuuaggauccacagggagggggcauuuuaauaucaaggagaacuaaucauc

SEQ ID NO: 1264 - RNA, 100 nt, Artificial Sequence
  Description of Artificial Sequence Synthetic polynucleotide
gauauugugacauaucucugcacucaucacacaaaagaugcaaacauaugauaggauccacagggagggggcauuuuaacauuuaauagaacuaaucauc

SEQ ID NO: 1265 - RNA, 100 nt, Artificial Sequence
  Description of Artificial Sequence Synthetic polynucleotide
gauauugugacauaucucugcacucaucacacaaaagaugcaaaagucaaauaggauccacagggagggggcauuucaauauccugcggaacuaaucauc

SEQ ID NO: 1266 - RNA, 100 nt, Artificial Sequence
  Description of Artificial Sequence Synthetic polynucleotide
gauauugugacauaucucugcacucaucacacaaaagaugcaaaccccgcauaggauccacagggagggggcauucuaauauccgagagaacuaaucauc

SEQ ID NO: 1267 - RNA, 100 nt, Artificial Sequence
  Description of Artificial Sequence Synthetic polynucleotide
gauauugugacauaucucugcacucaucacacaaaagaugcaaacacgaaauaggauccacagggagggggcaucuuaauaucaaguagaacuaaucauc

SEQ ID NO: 1268 - RNA, 100 nt, Artificial Sequence
  Description of Artificial Sequence Synthetic polynucleotide
gauauugugacauaucucugcacucaucacacaaaagaugcaaagccagaauaggauccacagggagggggcauuuuaauaucgaguggaacuaaucauc

SEQ ID NO: 1269 - RNA, 100 nt, Artificial Sequence
  Description of Artificial Sequence Synthetic polynucleotide
gauauugugacauaucucugcacucaucacacaaaagaugcaauggggauauaggauccacagggagggggcauuuuaacauuggcgagaacuaaucauc

SEQ ID NO: 1270 - RNA, 100 nt, Artificial Sequence
  Description of Artificial Sequence Synthetic polynucleotide
gauauugugacauaucucugcacucaucacacaaaagaugcaacugguauauaggauccacagggagggggcauuucaauauuaacgcgaacuaaucauc

SEQ ID NO: 1271 - RNA, 100 nt, Artificial Sequence
  Description of Artificial Sequence Synthetic polynucleotide
gauauugugacauaucucugcacucaucacacaaaagaugcaacgggaauauaggauccacagggagggggcauucuaauauccaccagaacuaaucauc

SEQ ID NO: 1272 - RNA, 100 nt, Artificial Sequence
  Description of Artificial Sequence Synthetic polynucleotide
gauauugugacauaucucugcacucaucacacaaaagaugcaaucaacguauaggauccacagggagggggcaucuuaauauuugagagaacuaaucauc

SEQ ID NO: 1273 - RNA, 100 nt, Artificial Sequence
  Description of Artificial Sequence Synthetic polynucleotide
gauauugugacauaucucugcacucaucacacaaaagaugcauugacccuauaggauccacagggagggggcauuuuaacauuagccggaacuaaucauc

SEQ ID NO: 1274 - RNA, 100 nt, Artificial Sequence
  Description of Artificial Sequence Synthetic polynucleotide
gauauugugacauaucucugcacucaucacacaaaagaugcauaaaaccuauaggauccacagggagggggcauuucaauaucguauggaacuaaucauc

SEQ ID NO: 1275 - RNA, 100 nt, Artificial Sequence
  Description of Artificial Sequence Synthetic polynucleotide
gauauugugacauaucucugcacucaucacacaaaagaugcauucaugcuauaggauccacagggagggggcauucuaauauuaaacagaacuaaucauc

SEQ ID NO: 1276 - RNA, 100 nt, Artificial Sequence
  Description of Artificial Sequence Synthetic polynucleotide
gauauugugacauaucucugcacucaucacacaaaagaugcaugcagccuauaggauccacagggagggggcaucuuaauauuguauggaacuaaucauc

SEQ ID NO: 1277 - RNA, 100 nt, Artificial Sequence
  Description of Artificial Sequence Synthetic polynucleotide
gauauugugacauaucucugcacucaucacacaaaagaugcauuagggcuauaggauccacagggagggggcauuuuaauauucaggagaacuaaucauc

SEQ ID NO: 1278 - RNA, 100 nt, Artificial Sequence
  Description of Artificial Sequence Synthetic polynucleotide
gauauugugacauaucucugcacucaucacacaaaagaugccccuacucuauaggauccacagggagggggcauuuuaacauuguagagaacuaaucauc

SEQ ID NO: 1279 - RNA, 100 nt, Artificial Sequence
  Description of Artificial Sequence Synthetic polynucleotide
gauauugugacauaucucugcacucaucacacaaaagaugcuuacaaucuauaggauccacagggagggggcauuucaauauccugcggaacuaaucauc

SEQ ID NO: 1280 - RNA, 100 nt, Artificial Sequence
  Description of Artificial Sequence Synthetic polynucleotide
gauauugugacauaucucugcacucaucacacaaaagaugcucgggcucuauaggauccacagggagggggcaucuuaauauugaccggaacuaaucauc

SEQ ID NO: 1281 - RNA, 100 nt, Artificial Sequence
  Description of Artificial Sequence Synthetic polynucleotide
gauauugugacauaucucugcacucaucacacaaaagaugcccaaccucuauaggauccacagggagggggcauuuuaauaucaaugggaacuaaucauc

SEQ ID NO: 1282 - RNA, 100 nt, Artificial Sequence
  Description of Artificial Sequence Synthetic polynucleotide
gauauugugacauaucucugcacucaucacacaaaagaugguuuaguucuauaggauccacagggagggggcauuuuaacauuggcgggaacuaaucauc

SEQ ID NO: 1283 - RNA, 100 nt, Artificial Sequence
  Description of Artificial Sequence Synthetic polynucleotide
gauauugugacauaucucugcacucaucacacaaaagaugacugcguucuauaggauccacagggagggggcauuucaauauucagaagaacuaaucauc

SEQ ID NO: 1284 - RNA, 100 nt, Artificial Sequence
  Description of Artificial Sequence Synthetic polynucleotide
gauauugugacauaucucugcacucaucacacaaaagaugauacgguucuauaggauccacagggagggggcauucuaauauuugaaagaacuaaucauc

SEQ ID NO: 1285 - RNA, 100 nt, Artificial Sequence
  Description of Artificial Sequence Synthetic polynucleotide
gauauugugacauaucucugcacucaucacacaaaagauggcgugauucuauaggauccacagggagggggcaucuuaauauccaauggaacuaaucauc

SEQ ID NO: 1286 - RNA, 100 nt, Artificial Sequence
  Description of Artificial Sequence Synthetic polynucleotide
gauauugugacauaucucugcacucaucacacaaaagaugaguaaauucuauaggauccacagggagggggcauuuuaauaucgaacggaacuaaucauc

SEQ ID NO: 1287 - RNA, 100 nt, Artificial Sequence
  Description of Artificial Sequence Synthetic polynucleotide
gauauugugacauaucucugcacucaucacacaaaagauaauauacuucuauaggauccacagggagggggcauuuuaacauuaggacgaacuaaucauc

SEQ ID NO: 1288 - RNA, 100 nt, Artificial Sequence
  Description of Artificial Sequence Synthetic polynucleotide
gauauugugacauaucucugcacucaucacacaaaagaucuucgccuucuauaggauccacagggagggggcauuucaauaucauagggaacuaaucauc

SEQ ID NO: 1289 - RNA, 100 nt, Artificial Sequence
  Description of Artificial Sequence Synthetic polynucleotide
gauauugugacauaucucugcacucaucacacaaaagaucuccgccuucuauaggauccacagggagggggcauucuaauaucagcgagaacuaaucauc

SEQ ID NO: 1290 - RNA, 100 nt, Artificial Sequence
  Description of Artificial Sequence Synthetic polynucleotide
gauauugugacauaucucugcacucaucacacaaaagauauugugcuucuauaggauccacagggagggggcaucuuaauauucacaggaacuaaucauc

SEQ ID NO: 1291 - RNA, 100 nt, Artificial Sequence
  Description of Artificial Sequence Synthetic polynucleotide
gauauugugacauaucucugcacucaucacacaaaagaucuacaccuucuauaggauccacagggagggggcauuuuaauaucaggaggaacuaaucauc

SEQ ID NO: 1292 - RNA, 100 nt, Artificial Sequence
  Description of Artificial Sequence Synthetic polynucleotide
gauauugugacauaucucugcacucaucacacaaaagagucuacucuucuauaggauccacagggagggggcauuuuaacauuaaguagaacuaaucauc

SEQ ID NO: 1293 - RNA, 100 nt, Artificial Sequence
  Description of Artificial Sequence Synthetic polynucleotide
gauauugugacauaucucugcacucaucacacaaaagaggguccucuucuauaggauccacagggagggggcauuucaauauccaggggaacuaaucauc

SEQ ID NO: 1294 - RNA, 100 nt, Artificial Sequence
  Description of Artificial Sequence Synthetic polynucleotide
gauauugugacauaucucugcacucaucacacaaaagaccccucucuucuauaggauccacagggagggggcauucuaauauucaaacgaacuaaucauc

SEQ ID NO: 1295 - RNA, 100 nt, Artificial Sequence
  Description of Artificial Sequence Synthetic polynucleotide
gauauugugacauaucucugcacucaucacacaaaagaacauucucuucuauaggauccacagggagggggcaucuuaauaucacagcgaacuaaucauc

SEQ ID NO: 1296 - RNA, 100 nt, Artificial Sequence
  Description of Artificial Sequence Synthetic polynucleotide
gauauugugacauaucucugcacucaucacacaaaagaaaaaccucuucuauaggauccacagggagggggcauuuuaauauuggauggaacuaaucauc

SEQ ID NO: 1297 - RNA, 100 nt, Artificial Sequence
  Description of Artificial Sequence Synthetic polynucleotide
gauauugugacauaucucugcacucaucacacaaaaguccguuaucuucuauaggauccacagggagggggcauuuuaacauuacaacgaacuaaucauc

SEQ ID NO: 1298 - RNA, 100 nt, Artificial Sequence
  Description of Artificial Sequence Synthetic polynucleotide
gauauugugacauaucucugcacucaucacacaaaaguacuucaucuucuauaggauccacagggagggggcauuucaauauuggcacgaacuaaucauc

SEQ ID NO: 1299 - RNA, 100 nt, Artificial Sequence
  Description of Artificial Sequence Synthetic polynucleotide
gauauugugacauaucucugcacucaucacacaaaagcaaauuaucuucuauaggauccacagggagggggcauucuaauauuggggagaacuaaucauc

SEQ ID NO: 1300 - RNA, 100 nt, Artificial Sequence
  Description of Artificial Sequence Synthetic polynucleotide
gauauugugacauaucucugcacucaucacacaaaaguaagauaucuucuauaggauccacagggagggggcaucuuaauauccaguggaacuaaucauc

SEQ ID NO: 1301 - RNA, 100 nt, Artificial Sequence
  Description of Artificial Sequence Synthetic polynucleotide
gauauugugacauaucucugcacucaucacacaaaagcguuuuaucuucuauaggauccacagggagggggcauuuuaauauuaguacgaacuaaucauc

SEQ ID NO: 1302 - RNA, 100 nt, Artificial Sequence
  Description of Artificial Sequence Synthetic polynucleotide
gauauugugacauaucucugcacucaucacacaaaauuauucaaucuucuauaggauccacagggagggggcauuuuaacauuaacgagaacuaaucauc

SEQ ID NO: 1303 - RNA, 100 nt, Artificial Sequence
  Description of Artificial Sequence Synthetic polynucleotide
gauauugugacauaucucugcacucaucacacaaaaaucuccaaucuucuauaggauccacagggagggggcauuucaauaucaaggggaacuaaucauc

SEQ ID NO: 1304 - RNA, 100 nt, Artificial Sequence
  Description of Artificial Sequence Synthetic polynucleotide
gauauugugacauaucucugcacucaucacacaaaacuaucuaaucuucuauaggauccacagggagggggcauucuaauauuuggacgaacuaaucauc

SEQ ID NO: 1305 - RNA, 100 nt, Artificial Sequence
  Description of Artificial Sequence Synthetic polynucleotide
gauauugugacauaucucugcacucaucacacaaaaccuaguaaucuucuauaggauccacagggagggggcaucuuaauauuugagagaacuaaucauc

SEQ ID NO: 1306 - RNA, 100 nt, Artificial Sequence
  Description of Artificial Sequence Synthetic polynucleotide
gauauugugacauaucucugcacucaucacacaaaacaacguaaucuucuauaggauccacagggagggggcauuuuaauaucgagaagaacuaaucauc

SEQ ID NO: 1307 - RNA, 100 nt, Artificial Sequence
  Description of Artificial Sequence Synthetic polynucleotide
gauauugugacauaucucugcacucaucacacaaacgcaaaaaaucuucuauaggauccacagggagggggcauuuuaacauuguaacgaacuaaucauc

SEQ ID NO: 1308 - RNA, 100 nt, Artificial Sequence
  Description of Artificial Sequence Synthetic polynucleotide
gauauugugacauaucucugcacucaucacacaaauaucagaaaucuucuauaggauccacagggagggggcauuucaauaucgagacgaacuaaucauc

SEQ ID NO: 1309 - RNA, 100 nt, Artificial Sequence
  Description of Artificial Sequence Synthetic polynucleotide
gauauugugacauaucucugcacucaucacacaaacacuagaaaucuucuauaggauccacagggagggggcauucuaauauccgagcgaacuaaucauc

SEQ ID NO: 1310 - RNA, 100 nt, Artificial Sequence
  Description of Artificial Sequence Synthetic polynucleotide
gauauugugacauaucucugcacucaucacacaaauuauuaaaaucuucuauaggauccacagggagggggcaucuuaauauuggguggaacuaaucauc

SEQ ID NO: 1311 - RNA, 100 nt, Artificial Sequence
  Description of Artificial Sequence Synthetic polynucleotide
gauauugugacauaucucugcacucaucacacaaaucaacgaaaucuucuauaggauccacagggagggggcauuuuaauauccaagcgaacuaaucauc

SEQ ID NO: 1312 - RNA, 100 nt, Artificial Sequence
  Description of Artificial Sequence Synthetic polynucleotide
gauauugugacauaucucugcacucaucacacaaucaccccaaaucuucuauaggauccacagggagggggcauuuuaacauuagacggaacuaaucauc

SEQ ID NO: 1313 - RNA, 100 nt, Artificial Sequence
  Description of Artificial Sequence Synthetic polynucleotide
gauauugugacauaucucugcacucaucacacaaccuaaucaaaucuucuauaggauccacagggagggggcauuucaauaucgacgcgaacuaaucauc

SEQ ID NO: 1314 - RNA, 100 nt, Artificial Sequence
  Description of Artificial Sequence Synthetic polynucleotide
gauauugugacauaucucugcacucaucacacaacuccaucaaaucuucuauaggauccacagggagggggcauucuaauaucgaccagaacuaaucauc

SEQ ID NO: 1315 - RNA, 100 nt, Artificial Sequence
  Description of Artificial Sequence Synthetic polynucleotide
gauauugugacauaucucugcacucaucacacaacaauuucaaaucuucuauaggauccacagggagggggcaucuuaauaucagcaagaacuaaucauc

SEQ ID NO: 1316 - RNA, 100 nt, Artificial Sequence
  Description of Artificial Sequence Synthetic polynucleotide
gauauugugacauaucucugcacucaucacacaaccugcccaaaucuucuauaggauccacagggagggggcauuuuaauauuccagagaacuaaucauc

SEQ ID NO: 1317 - RNA, 100 nt, Artificial Sequence
  Description of Artificial Sequence Synthetic polynucleotide
gauauugugacauaucucugcacucaucacacaccgugcgcaaaucuucuauaggauccacagggagggggcauuuuaacauuacaacgaacuaaucauc

SEQ ID NO: 1318 - RNA, 100 nt, Artificial Sequence
  Description of Artificial Sequence Synthetic polynucleotide
gauauugugacauaucucugcacucaucacacauuauacgcaaaucuucuauaggauccacagggagggggcauuucaauauuuacgggaacuaaucauc

SEQ ID NO: 1319 - RNA, 100 nt, Artificial Sequence
  Description of Artificial Sequence Synthetic polynucleotide
gauauugugacauaucucugcacucaucacacacuaaccgcaaaucuucuauaggauccacagggagggggcauucuaauauuagcacgaacuaaucauc

SEQ ID NO: 1320 - RNA, 100 nt, Artificial Sequence
  Description of Artificial Sequence Synthetic polynucleotide
gauauugugacauaucucugcacucaucacacauuuguagcaaaucuucuauaggauccacagggagggggcaucuuaauaucaaggcgaacuaaucauc

SEQ ID NO: 1321 - RNA, 100 nt, Artificial Sequence
  Description of Artificial Sequence Synthetic polynucleotide
gauauugugacauaucucugcacucaucacacaugcucagcaaaucuucuauaggauccacagggagggggcauuuuaauaucagugggaacuaaucauc

SEQ ID NO: 1322 - RNA, 100 nt, Artificial Sequence
  Description of Artificial Sequence Synthetic polynucleotide
gauauugugacauaucucugcacucaucacacuccuauugcaaaucuucuauaggauccacagggagggggcauuuuaacauuuaguggaacuaaucauc

SEQ ID NO: 1323 - RNA, 100 nt, Artificial Sequence
  Description of Artificial Sequence Synthetic polynucleotide
gauauugugacauaucucugcacucaucacacuguaccugcaaaucuucuauaggauccacagggagggggcauuucaauaucagcaggaacuaaucauc

SEQ ID NO: 1324 - RNA, 100 nt, Artificial Sequence
  Description of Artificial Sequence Synthetic polynucleotide
gauauugugacauaucucugcacucaucacacucccucugcaaaucuucuauaggauccacagggagggggcauucuaauauuccaaagaacuaaucauc

SEQ ID NO: 1325 - RNA, 100 nt, Artificial Sequence
  Description of Artificial Sequence Synthetic polynucleotide
gauauugugacauaucucugcacucaucacacuuccguugcaaaucuucuauaggauccacagggagggggcaucuuaauauccgcaagaacuaaucauc

SEQ ID NO: 1326 - RNA, 100 nt, Artificial Sequence
  Description of Artificial Sequence Synthetic polynucleotide
gauauugugacauaucucugcacucaucacaccggccuugcaaaucuucuauaggauccacagggagggggcauuuuaauaucaaggagaacuaaucauc

SEQ ID NO: 1327 - RNA, 100 nt, Artificial Sequence
  Description of Artificial Sequence Synthetic polynucleotide
gauauugugacauaucucugcacucaucacaauggcaaugcaaaucuucuauaggauccacagggagggggcauuuuaacauuaacaagaacuaaucauc

SEQ ID NO: 1328 - RNA, 100 nt, Artificial Sequence
  Description of Artificial Sequence Synthetic polynucleotide
gauauugugacauaucucugcacucaucacaaaucaaaugcaaaucuucuauaggauccacagggagggggcauuucaauauuuggacgaacuaaucauc

SEQ ID NO: 1329 - RNA, 100 nt, Artificial Sequence
  Description of Artificial Sequence Synthetic polynucleotide
gauauugugacauaucucugcacucaucacagccauaaugcaaaucuucuauaggauccacagggagggggcauucuaauauuaccgcgaacuaaucauc

SEQ ID NO: 1330 - RNA, 100 nt, Artificial Sequence
  Description of Artificial Sequence Synthetic polynucleotide
gauauugugacauaucucugcacucaucacagucaaaaugcaaaucuucuauaggauccacagggagggggcaucuuaauaucgaggagaacuaaucauc

SEQ ID NO: 1331 - RNA, 100 nt, Artificial Sequence
  Description of Artificial Sequence Synthetic polynucleotide
gauauugugacauaucucugcacucaucacaauccucaugcaaaucuucuauaggauccacagggagggggcauuuuaauaucaugaggaacuaaucauc

SEQ ID NO: 1332 - RNA, 100 nt, Artificial Sequence
  Description of Artificial Sequence Synthetic polynucleotide
gauauugugacauaucucugcacucaucacuaucaugaugcaaaucuucuauaggauccacagggagggggcauuucaauauccgggagaacuaaucauc

SEQ ID NO: 1333 - RNA, 100 nt, Artificial Sequence
  Description of Artificial Sequence Synthetic polynucleotide
gauauugugacauaucucugcacucaucacuacccugaugcaaaucuucuauaggauccacagggagggggcauucuaauaucaccaggaacuaaucauc

SEQ ID NO: 1334 - RNA, 100 nt, Artificial Sequence
  Description of Artificial Sequence Synthetic polynucleotide
gauauugugacauaucucugcacucaucaccugucugaugcaaaucuucuauaggauccacagggagggggcaucuuaauaucguauagaacuaaucauc

SEQ ID NO: 1335 - RNA, 100 nt, Artificial Sequence
  Description of Artificial Sequence Synthetic polynucleotide
gauauugugacauaucucugcacucaucaccugguugaugcaaaucuucuauaggauccacagggagggggcauuuuaauaucacacggaacuaaucauc

SEQ ID NO: 1336 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaauaaugacgugaacuaaucauc

SEQ ID NO: 1337 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauaaugaggugaacuaaucauc

SEQ ID NO: 1338 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauaaugaaaugaacuaaucauc

SEQ ID NO: 1339 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauaaacgcaugaacuaaucauc

SEQ ID NO: 1340 - RNA, 100 nt, Artificial Sequence
  Description of Artificial Sequence Synthetic polynucleotide
gauauugugacauaucucugcacucaucacacaaaagaugcaaaucuucuccuaccuccacagggagggggcauuucaauagccacgugaacuaaucauc

SEQ ID NO: 1341 - RNA, 100 nt, Artificial Sequence
  Description of Artificial Sequence Synthetic polynucleotide
gauauugugacauaucucugcacucaucacacaaaagaugcaaaucuucuccccacuccacagggagggggcauucuaauaaccacaugaacuaaucauc

SEQ ID NO: 1342 - RNA, 100 nt, Artificial Sequence
  Description of Artificial Sequence Synthetic polynucleotide
gauauugugacauaucucugcacucaucacacaaaagaugcaaaucuucuuccauuuccacagggagggggcaucuuaauaaccaggugaacuaaucauc

SEQ ID NO: 1343 - RNA, 100 nt, Artificial Sequence
  Description of Artificial Sequence Synthetic polynucleotide
gauauugugacauaucucugcacucaucacacaaaagaugcaaaucuucucgcuucuccacagggagggggcauuuuaauacuggaaugaacuaaucauc

SEQ ID NO: 1344 - RNA, 100 nt, Artificial Sequence
  Description of Artificial Sequence Synthetic polynucleotide
gauauugugacauaucucugcacucaucacacaaaagaugcaaaucuucgauccuauccacagggagggggcauuucaauaaacaagugaacuaaucauc

SEQ ID NO: 1345 - RNA, 100 nt, Artificial Sequence
  Description of Artificial Sequence Synthetic polynucleotide
gauauugugacauaucucugcacucaucacacaaaagaugcaaaucuucgcagucauccacagggagggggcauucuaauaccagcaugaacuaaucauc

SEQ ID NO: 1346 - RNA, 100 nt, Artificial Sequence
  Description of Artificial Sequence Synthetic polynucleotide
gauauugugacauaucucugcacucaucacacaaaagaugcaaaucuucaacauaauccacagggagggggcaucuuaauagcgaagugaacuaaucauc

SEQ ID NO: 1347 - RNA, 100 nt, Artificial Sequence
  Description of Artificial Sequence Synthetic polynucleotide
gauauugugacauaucucugcacucaucacacaaaagaugcaaaucuucguaaaaauccacagggagggggcauuuuaauacgggggugaacuaaucauc

SEQ ID NO: 1348 - RNA, 100 nt, Artificial Sequence
  Description of Artificial Sequence Synthetic polynucleotide
gauauugugacauaucucugcacucaucacacaaaagaugcaaaucuugauaaugauccacagggagggggcauuucaauaguauaaugaacuaaucauc

SEQ ID NO: 1349 - RNA, 100 nt, Artificial Sequence
  Description of Artificial Sequence Synthetic polynucleotide
gauauugugacauaucucugcacucaucacacaaaagaugcaaaucuuagucuugauccacagggagggggcauucuaauaacgacaugaacuaaucauc

SEQ ID NO: 1350 - RNA, 100 nt, Artificial Sequence
  Description of Artificial Sequence Synthetic polynucleotide
gauauugugacauaucucugcacucaucacacaaaagaugcaaaucuuggcgcugauccacagggagggggcaucuuaauagcaauaugaacuaaucauc

SEQ ID NO: 1351 - RNA, 100 nt, Artificial Sequence
  Description of Artificial Sequence Synthetic polynucleotide
gauauugugacauaucucugcacucaucacacaaaagaugcaaaucuuggcacugauccacagggagggggcauuuuaauaggauagugaacuaaucauc

SEQ ID NO: 1352 - RNA, 100 nt, Artificial Sequence
  Description of Artificial Sequence Synthetic polynucleotide
gauauugugacauaucucugcacucaucacacaaaagaugcaaaucuauacucggauccacagggagggggcauuucaauaaaaguaugaacuaaucauc

SEQ ID NO: 1353 - RNA, 100 nt, Artificial Sequence
  Description of Artificial Sequence Synthetic polynucleotide
gauauugugacauaucucugcacucaucacacaaaagaugcaaaucugagcguggauccacagggagggggcauucuaauaguggaaugaacuaaucauc

SEQ ID NO: 1354 - RNA, 100 nt, Artificial Sequence
  Description of Artificial Sequence Synthetic polynucleotide
gauauugugacauaucucugcacucaucacacaaaagaugcaaaucuguucgcggauccacagggagggggcaucuuaauacaauggugaacuaaucauc

SEQ ID NO: 1355 - RNA, 100 nt, Artificial Sequence
  Description of Artificial Sequence Synthetic polynucleotide
gauauugugacauaucucugcacucaucacacaaaagaugcaaaucuaacauuggauccacagggagggggcauuuuaauacccgagugaacuaaucauc

SEQ ID NO: 1356 - RNA, 100 nt, Artificial Sequence
  Description of Artificial Sequence Synthetic polynucleotide
gauauugugacauaucucugcacucaucacacaaaagaugcaaaucgcacaaaggauccacagggagggggcauucuaauagggacaugaacuaaucauc

SEQ ID NO: 1357 - RNA, 100 nt, Artificial Sequence
  Description of Artificial Sequence Synthetic polynucleotide
gauauugugacauaucucugcacucaucacacaaaagaugcaaaucaaguucaggauccacagggagggggcaucuuaauaguagaaugaacuaaucauc

SEQ ID NO: 1358 - RNA, 100 nt, Artificial Sequence
  Description of Artificial Sequence Synthetic polynucleotide
gauauugugacauaucucugcacucaucacacaaaagaugcaaauccgacaaaggauccacagggagggggcauuuuaauaggguuaugaacuaaucauc

SEQ ID NO: 1359 - RNA, 100 nt, Artificial Sequence
  Description of Artificial Sequence Synthetic polynucleotide
gauauugugacauaucucugcacucaucacacaaaagaugcaaauauguauuaggauccacagggagggggcauuucaauagcuacaugaacuaaucauc

SEQ ID NO: 1360 - RNA, 100 nt, Artificial Sequence
  Description of Artificial Sequence Synthetic polynucleotide
gauauugugacauaucucugcacucaucacacaaaagaugcaaaugaggccuaggauccacagggagggggcauucuaauaagcuagugaacuaaucauc

SEQ ID NO: 1361 - RNA, 100 nt, Artificial Sequence
  Description of Artificial Sequence Synthetic polynucleotide
gauauugugacauaucucugcacucaucacacaaaagaugcaaauaauggcuaggauccacagggagggggcaucuuaauaccauaaugaacuaaucauc

SEQ ID NO: 1362 - RNA, 100 nt, Artificial Sequence
  Description of Artificial Sequence Synthetic polynucleotide
gauauugugacauaucucugcacucaucacacaaaagaugcaaauacaaguuaggauccacagggagggggcauuuuaauacgcgagugaacuaaucauc

SEQ ID NO: 1363 - RNA, 100 nt, Artificial Sequence
  Description of Artificial Sequence Synthetic polynucleotide
gauauugugacauaucucugcacucaucacacaaaagaugcaaaaagcgaauaggauccacagggagggggcauuucaauacagguaugaacuaaucauc

SEQ ID NO: 1364 - RNA, 100 nt, Artificial Sequence
  Description of Artificial Sequence Synthetic polynucleotide
gauauugugacauaucucugcacucaucacacaaaagaugcaaaguauugauaggauccacagggagggggcauucuaauaguguggugaacuaaucauc

SEQ ID NO: 1365 - RNA, 100 nt, Artificial Sequence
  Description of Artificial Sequence Synthetic polynucleotide
gauauugugacauaucucugcacucaucacacaaaagaugcaaaagguacauaggauccacagggagggggcaucuuaauacagagaugaacuaaucauc

SEQ ID NO: 1366 - RNA, 100 nt, Artificial Sequence
  Description of Artificial Sequence Synthetic polynucleotide
gauauugugacauaucucugcacucaucacacaaaagaugcaaacgccacauaggauccacagggagggggcauuuuaauagaaugaugaacuaaucauc

SEQ ID NO: 1367 - RNA, 100 nt, Artificial Sequence
  Description of Artificial Sequence Synthetic polynucleotide
gauauugugacauaucucugcacucaucacacaaaagaugcaaccgcaauauaggauccacagggagggggcauuucaauaacuacaugaacuaaucauc

SEQ ID NO: 1368 - RNA, 100 nt, Artificial Sequence
  Description of Artificial Sequence Synthetic polynucleotide
gauauugugacauaucucugcacucaucacacaaaagaugcaacaaacguauaggauccacagggagggggcauucuaauacacgcgugaacuaaucauc

SEQ ID NO: 1369 - RNA, 100 nt, Artificial Sequence
  Description of Artificial Sequence Synthetic polynucleotide
gauauugugacauaucucugcacucaucacacaaaagaugcaacugcgguauaggauccacagggagggggcaucuuaauacccgggugaacuaaucauc

SEQ ID NO: 1370 - RNA, 100 nt, Artificial Sequence
  Description of Artificial Sequence Synthetic polynucleotide
gauauugugacauaucucugcacucaucacacaaaagaugcaacacacauauaggauccacagggagggggcauuuuaauagcgggaugaacuaaucauc

SEQ ID NO: 1371 - RNA, 100 nt, Artificial Sequence
  Description of Artificial Sequence Synthetic polynucleotide
gauauugugacauaucucugcacucaucacacaaaagaugcauuagggcuauaggauccacagggagggggcauuucaauaaugaggugaacuaaucauc

SEQ ID NO: 1372 - RNA, 100 nt, Artificial Sequence
  Description of Artificial Sequence Synthetic polynucleotide
gauauugugacauaucucugcacucaucacacaaaagaugcaugguaccuauaggauccacagggagggggcauucuaauagccaaaugaacuaaucauc

SEQ ID NO: 1373 - RNA, 100 nt, Artificial Sequence
  Description of Artificial Sequence Synthetic polynucleotide
gauauugugacauaucucugcacucaucacacaaaagaugcaucaaaacuauaggauccacagggagggggcaucuuaauacgaaagugaacuaaucauc

SEQ ID NO: 1374 - RNA, 100 nt, Artificial Sequence
  Description of Artificial Sequence Synthetic polynucleotide
gauauugugacauaucucugcacucaucacacaaaagaugcacgaccccuauaggauccacagggagggggcauuuuaauacgagggugaacuaaucauc

SEQ ID NO: 1375 - RNA, 100 nt, Artificial Sequence
  Description of Artificial Sequence Synthetic polynucleotide
gauauugugacauaucucugcacucaucacacaaaagaugccgcaaaucuauaggauccacagggagggggcauuucaauaguggcaugaacuaaucauc

SEQ ID NO: 1376 - RNA, 100 nt, Artificial Sequence
  Description of Artificial Sequence Synthetic polynucleotide
gauauugugacauaucucugcacucaucacacaaaagaugcccggagucuauaggauccacagggagggggcauucuaauagaccaaugaacuaaucauc

SEQ ID NO: 1377 - RNA, 100 nt, Artificial Sequence
  Description of Artificial Sequence Synthetic polynucleotide
gauauugugacauaucucugcacucaucacacaaaagaugcucaaaaucuauaggauccacagggagggggcaucuuaauaggggcaugaacuaaucauc

SEQ ID NO: 1378 - RNA, 100 nt, Artificial Sequence
  Description of Artificial Sequence Synthetic polynucleotide
gauauugugacauaucucugcacucaucacacaaaagaugcuacauaucuauaggauccacagggagggggcauuuuaauaguagcgugaacuaaucauc

SEQ ID NO: 1379 - RNA, 100 nt, Artificial Sequence
  Description of Artificial Sequence Synthetic polynucleotide
gauauugugacauaucucugcacucaucacacaaaagauggccuaguucuauaggauccacagggagggggcauuucaauaacccaaugaacuaaucauc

SEQ ID NO: 1380 - RNA, 100 nt, Artificial Sequence
  Description of Artificial Sequence Synthetic polynucleotide
gauauugugacauaucucugcacucaucacacaaaagaugguuugauucuauaggauccacagggagggggcauucuaauaaaaguaugaacuaaucauc

SEQ ID NO: 1381 - RNA, 100 nt, Artificial Sequence
  Description of Artificial Sequence Synthetic polynucleotide
gauauugugacauaucucugcacucaucacacaaaagaugguuacauucuauaggauccacagggagggggcaucuuaauaacagcaugaacuaaucauc

SEQ ID NO: 1382 - RNA, 100 nt, Artificial Sequence
  Description of Artificial Sequence Synthetic polynucleotide
gauauugugacauaucucugcacucaucacacaaaagaugaaucaauucuauaggauccacagggagggggcauuuuaauaaccacgugaacuaaucauc

SEQ ID NO: 1383 - RNA, 100 nt, Artificial Sequence
  Description of Artificial Sequence Synthetic polynucleotide
gauauugugacauaucucugcacucaucacacaaaagauuccguccuucuauaggauccacagggagggggcauuucaauaaugacaugaacuaaucauc

SEQ ID NO: 1384 - RNA, 100 nt, Artificial Sequence
  Description of Artificial Sequence Synthetic polynucleotide
gauauugugacauaucucugcacucaucacacaaaagauaaguggcuucuauaggauccacagggagggggcauucuaauagcaaaaugaacuaaucauc

SEQ ID NO: 1385 - RNA, 100 nt, Artificial Sequence
  Description of Artificial Sequence Synthetic polynucleotide
gauauugugacauaucucugcacucaucacacaaaagauaacguccuucuauaggauccacagggagggggcaucuuaauaacugagugaacuaaucauc

SEQ ID NO: 1386 - RNA, 100 nt, Artificial Sequence
  Description of Artificial Sequence Synthetic polynucleotide
gauauugugacauaucucugcacucaucacacaaaagaucuucaccuucuauaggauccacagggagggggcauuuuaauacgguggugaacuaaucauc

SEQ ID NO: 1387 - RNA, 100 nt, Artificial Sequence
  Description of Artificial Sequence Synthetic polynucleotide
gauauugugacauaucucugcacucaucacacaaaagaccguuuucuucuauaggauccacagggagggggcauuucaauacggagaugaacuaaucauc

SEQ ID NO: 1388 - RNA, 100 nt, Artificial Sequence
  Description of Artificial Sequence Synthetic polynucleotide
gauauugugacauaucucugcacucaucacacaaaagacuagucucuucuauaggauccacagggagggggcauucuaauagcugggugaacuaaucauc

SEQ ID NO: 1389 - RNA, 100 nt, Artificial Sequence
  Description of Artificial Sequence Synthetic polynucleotide
gauauugugacauaucucugcacucaucacacaaaagaacacuuucuucuauaggauccacagggagggggcaucuuaauagugggaugaacuaaucauc

SEQ ID NO: 1390 - RNA, 100 nt, Artificial Sequence
  Description of Artificial Sequence Synthetic polynucleotide
gauauugugacauaucucugcacucaucacacaaaagagaccuuucuucuauaggauccacagggagggggcauuuuaauagaauaaugaacuaaucauc

SEQ ID NO: 1391 - RNA, 100 nt, Artificial Sequence
  Description of Artificial Sequence Synthetic polynucleotide
gauauugugacauaucucugcacucaucacacaaaagcacccuaucuucuauaggauccacagggagggggcauuucaauagcggagugaacuaaucauc

SEQ ID NO: 1392 - RNA, 100 nt, Artificial Sequence
  Description of Artificial Sequence Synthetic polynucleotide
gauauugugacauaucucugcacucaucacacaaaagucauuuaucuucuauaggauccacagggagggggcauucuaauacccuagugaacuaaucauc

SEQ ID NO: 1393 - RNA, 100 nt, Artificial Sequence
  Description of Artificial Sequence Synthetic polynucleotide
gauauugugacauaucucugcacucaucacacaaaaguuacccaucuucuauaggauccacagggagggggcauuuuaauaggcagaugaacuaaucauc

SEQ ID NO: 1394 - RNA, 100 nt, Artificial Sequence
  Description of Artificial Sequence Synthetic polynucleotide
gauauugugacauaucucugcacucaucacacaaaauuuuucaaucuucuauaggauccacagggagggggcauuucaauaacgaaaugaacuaaucauc

SEQ ID NO: 1395 - RNA, 100 nt, Artificial Sequence
  Description of Artificial Sequence Synthetic polynucleotide
gauauugugacauaucucugcacucaucacacaaaaucauauaaucuucuauaggauccacagggagggggcauucuaauaaaguggugaacuaaucauc

SEQ ID NO: 1396 - RNA, 100 nt, Artificial Sequence
  Description of Artificial Sequence Synthetic polynucleotide
gauauugugacauaucucugcacucaucacacaaaaauuaacaaucuucuauaggauccacagggagggggcaucuuaauacaauagugaacuaaucauc

SEQ ID NO: 1397 - RNA, 100 nt, Artificial Sequence
  Description of Artificial Sequence Synthetic polynucleotide
gauauugugacauaucucugcacucaucacacaaaaaacaauaaucuucuauaggauccacagggagggggcauuuuaauacugaggugaacuaaucauc

SEQ ID NO: 1398 - RNA, 100 nt, Artificial Sequence
  Description of Artificial Sequence Synthetic polynucleotide
gauauugugacauaucucugcacucaucacacaaauacacaaaaucuucuauaggauccacagggagggggcauuucaauagccgcaugaacuaaucauc

SEQ ID NO: 1399 - RNA, 100 nt, Artificial Sequence
  Description of Artificial Sequence Synthetic polynucleotide
gauauugugacauaucucugcacucaucacacaaauucccgaaaucuucuauaggauccacagggagggggcauucuaauagaagaaugaacuaaucauc

SEQ ID NO: 1400 - RNA, 100 nt, Artificial Sequence
  Description of Artificial Sequence Synthetic polynucleotide
gauauugugacauaucucugcacucaucacacaaacgcacgaaaucuucuauaggauccacagggagggggcaucuuaauaacagcgugaacuaaucauc

SEQ ID NO: 1401 - RNA, 100 nt, Artificial Sequence
  Description of Artificial Sequence Synthetic polynucleotide
gauauugugacauaucucugcacucaucacacaaugaauucaaaucuucuauaggauccacagggagggggcauuucaauaaaaaggugaacuaaucauc

SEQ ID NO: 1402 - RNA, 100 nt, Artificial Sequence
  Description of Artificial Sequence Synthetic polynucleotide
gauauugugacauaucucugcacucaucacacaacgcuuucaaaucuucuauaggauccacagggagggggcauucuaauacuagcaugaacuaaucauc

SEQ ID NO: 1403 - RNA, 100 nt, Artificial Sequence
  Description of Artificial Sequence Synthetic polynucleotide
gauauugugacauaucucugcacucaucacacaaugaauacaaaucuucuauaggauccacagggagggggcaucuuaauagacugaugaacuaaucauc

SEQ ID NO: 1404 - RNA, 100 nt, Artificial Sequence
  Description of Artificial Sequence Synthetic polynucleotide
gauauugugacauaucucugcacucaucacacaauuauuucaaaucuucuauaggauccacagggagggggcauuuuaauagugaaaugaacuaaucauc

SEQ ID NO: 1405 - RNA, 100 nt, Artificial Sequence
  Description of Artificial Sequence Synthetic polynucleotide
gauauugugacauaucucugcacucaucacacaccuagcgcaaaucuucuauaggauccacagggagggggcauuucaauaguguggugaacuaaucauc

SEQ ID NO: 1406 - RNA, 100 nt, Artificial Sequence
  Description of Artificial Sequence Synthetic polynucleotide
gauauugugacauaucucugcacucaucacacauuucuagcaaaucuucuauaggauccacagggagggggcauucuaauacgaggaugaacuaaucauc

SEQ ID NO: 1407 - RNA, 100 nt, Artificial Sequence
  Description of Artificial Sequence Synthetic polynucleotide
gauauugugacauaucucugcacucaucacacacgcaacgcaaaucuucuauaggauccacagggagggggcaucuuaauaccgacaugaacuaaucauc

SEQ ID NO: 1408 - RNA, 100 nt, Artificial Sequence
  Description of Artificial Sequence Synthetic polynucleotide
gauauugugacauaucucugcacucaucacacacuucgggcaaaucuucuauaggauccacagggagggggcauuuuaauacuagagugaacuaaucauc

SEQ ID NO: 1409 - RNA, 100 nt, Artificial Sequence
  Description of Artificial Sequence Synthetic polynucleotide
gauauugugacauaucucugcacucaucacaccgcacuugcaaaucuucuauaggauccacagggagggggcauuucaauacuggggugaacuaaucauc

SEQ ID NO: 1410 - RNA, 100 nt, Artificial Sequence
  Description of Artificial Sequence Synthetic polynucleotide
gauauugugacauaucucugcacucaucacaccccuacugcaaaucuucuauaggauccacagggagggggcauucuaauaggaggaugaacuaaucauc

SEQ ID NO: 1411 - RNA, 100 nt, Artificial Sequence
  Description of Artificial Sequence Synthetic polynucleotide
gauauugugacauaucucugcacucaucacacccucccugcaaaucuucuauaggauccacagggagggggcaucuuaauaagcgggugaacuaaucauc

SEQ ID NO: 1412 - RNA, 100 nt, Artificial Sequence
  Description of Artificial Sequence Synthetic polynucleotide
gauauugugacauaucucugcacucaucacaccuucccugcaaaucuucuauaggauccacagggagggggcauuuuaauagaauggugaacuaaucauc

SEQ ID NO: 1413 - RNA, 100 nt, Artificial Sequence
  Description of Artificial Sequence Synthetic polynucleotide
gauauugugacauaucucugcacucaucacaacuuccaugcaaaucuucuauaggauccacagggagggggcauuucaauaaggagaugaacuaaucauc

SEQ ID NO: 1414 - RNA, 100 nt, Artificial Sequence
  Description of Artificial Sequence Synthetic polynucleotide
gauauugugacauaucucugcacucaucacagcaccaaugcaaaucuucuauaggauccacagggagggggcauucuaauacggguaugaacuaaucauc

SEQ ID NO: 1415 - RNA, 100 nt, Artificial Sequence
  Description of Artificial Sequence Synthetic polynucleotide
gauauugugacauaucucugcacucaucacaacccauaugcaaaucuucuauaggauccacagggagggggcaucuuaauacaagagugaacuaaucauc

SEQ ID NO: 1416 - RNA, 100 nt, Artificial Sequence
  Description of Artificial Sequence Synthetic polynucleotide
gauauugugacauaucucugcacucaucacagcucucaugcaaaucuucuauaggauccacagggagggggcauuuuaauacaggcaugaacuaaucauc

SEQ ID NO: 1417 - RNA, 100 nt, Artificial Sequence
  Description of Artificial Sequence Synthetic polynucleotide
gauauugugacauaucucugcacucaucacuucuuugaugcaaaucuucuauaggauccacagggagggggcauuucaauaaggggaugaacuaaucauc

SEQ ID NO: 1418 - RNA, 100 nt, Artificial Sequence
  Description of Artificial Sequence Synthetic polynucleotide
gauauugugacauaucucugcacucaucacuccacugaugcaaaucuucuauaggauccacagggagggggcauucuaauaacaacgugaacuaaucauc

SEQ ID NO: 1419 - RNA, 100 nt, Artificial Sequence
  Description of Artificial Sequence Synthetic polynucleotide
gauauugugacauaucucugcacucaucaccuuuacgaugcaaaucuucuauaggauccacagggagggggcaucuuaauacacccaugaacuaaucauc

SEQ ID NO: 1420 - RNA, 100 nt, Artificial Sequence
  Description of Artificial Sequence Synthetic polynucleotide
gauauugugacauaucucugcacucaucacuauaucgaugcaaaucuucuauaggauccacagggagggggcauuuuaauacacugaugaacuaaucauc

SEQ ID NO: 1421 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaacccuggacugaacuaaucauc

SEQ ID NO: 1422 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuucaaucccaagcugaacuaaucauc

SEQ ID NO: 1423 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauucuaauucgcggcugaacuaaucauc

SEQ ID NO: 1424 - RNA, 100 nt, Artificial Sequence
  Description of Artificial Sequence Synthetic polynucleotide
gauauugugacauaucucugcacucaucacacaaaagaugcaaaucuucuauaggauccacagggagggggcaucuuaauccuggacugaacuaaucauc

SEQ ID NO: 1425 - RNA, 100 nt, Artificial Sequence
  Description of Artificial Sequence Synthetic polynucleotide
gauauugugacauaucucugcacucaucacacaaaagaugcaaaucuucuauaggauccacagggagggggcauuuuaauuacugacugaacuaaucauc

SEQ ID NO: 1426 - RNA, 100 nt, Artificial Sequence
  Description of Artificial Sequence Synthetic polynucleotide
gauauugugacauaucucugcacucaucacacaaaagaugcaaaucuucacuuucauccacagggagggggcauuuuaaccccuugcugaacuaaucauc

SEQ ID NO: 1427 - RNA, 100 nt, Artificial Sequence
  Description of Artificial Sequence Synthetic polynucleotide
gauauugugacauaucucugcacucaucacacaaaagaugcaaaucuuccucccuauccacagggagggggcauuucaaucggacgcugaacuaaucauc

SEQ ID NO: 1428 - RNA, 100 nt, Artificial Sequence
  Description of Artificial Sequence Synthetic polynucleotide
gauauugugacauaucucugcacucaucacacaaaagaugcaaaucuucacauauauccacagggagggggcauucuaauucugaccugaacuaaucauc

SEQ ID NO: 1429 - RNA, 100 nt, Artificial Sequence
  Description of Artificial Sequence Synthetic polynucleotide
gauauugugacauaucucugcacucaucacacaaaagaugcaaaucuuccaaucuauccacagggagggggcaucuuaauuaacaccugaacuaaucauc

SEQ ID NO: 1430 - RNA, 100 nt, Artificial Sequence
  Description of Artificial Sequence Synthetic polynucleotide
gauauugugacauaucucugcacucaucacacaaaagaugcaaaucuucggcgccauccacagggagggggcauuuuaaucgccgacugaacuaaucauc

SEQ ID NO: 1431 - RNA, 100 nt, Artificial Sequence
  Description of Artificial Sequence Synthetic polynucleotide
gauauugugacauaucucugcacucaucacacaaaagaugcaaaucuugagcuugauccacagggagggggcauuuuaacuuuaggcugaacuaaucauc

SEQ ID NO: 1432 - RNA, 100 nt, Artificial Sequence
  Description of Artificial Sequence Synthetic polynucleotide
gauauugugacauaucucugcacucaucacacaaaagaugcaaaucuuggucaugauccacagggagggggcauuucaaucugacacugaacuaaucauc

SEQ ID NO: 1433 - RNA, 100 nt, Artificial Sequence
  Description of Artificial Sequence Synthetic polynucleotide
gauauugugacauaucucugcacucaucacacaaaagaugcaaaucuuagcuccgauccacagggagggggcauucuaauuacggacugaacuaaucauc

SEQ ID NO: 1434 - RNA, 100 nt, Artificial Sequence
  Description of Artificial Sequence Synthetic polynucleotide
gauauugugacauaucucugcacucaucacacaaaagaugcaaaucuuacgauugauccacagggagggggcaucuuaauccggaccugaacuaaucauc

SEQ ID NO: 1435 - RNA, 100 nt, Artificial Sequence
  Description of Artificial Sequence Synthetic polynucleotide
gauauugugacauaucucugcacucaucacacaaaagaugcaaaucuugacgccgauccacagggagggggcauuuuaaucggcagcugaacuaaucauc

SEQ ID NO: 1436 - RNA, 100 nt, Artificial Sequence
  Description of Artificial Sequence Synthetic polynucleotide
gauauugugacauaucucugcacucaucacacaaaagaugcaaaucuagcccuggauccacagggagggggcauuuuaacgagcggcugaacuaaucauc

SEQ ID NO: 1437 - RNA, 100 nt, Artificial Sequence
  Description of Artificial Sequence Synthetic polynucleotide
gauauugugacauaucucugcacucaucacacaaaagaugcaaaucugauugcggauccacagggagggggcauuucaauuuuaggcugaacuaaucauc

SEQ ID NO: 1438 - RNA, 100 nt, Artificial Sequence
  Description of Artificial Sequence Synthetic polynucleotide
gauauugugacauaucucugcacucaucacacaaaagaugcaaaucuggaccuggauccacagggagggggcauucuaaucgcuagcugaacuaaucauc

SEQ ID NO: 1439 - RNA, 100 nt, Artificial Sequence
  Description of Artificial Sequence Synthetic polynucleotide
gauauugugacauaucucugcacucaucacacaaaagaugcaaaucugguuacggauccacagggagggggcaucuuaauuccggacugaacuaaucauc

SEQ ID NO: 1440 - RNA, 100 nt, Artificial Sequence
  Description of Artificial Sequence Synthetic polynucleotide
gauauugugacauaucucugcacucaucacacaaaagaugcaaaucucagucuggauccacagggagggggcauuuuaaucaaacacugaacuaaucauc

SEQ ID NO: 1441 - RNA, 100 nt, Artificial Sequence
  Description of Artificial Sequence Synthetic polynucleotide
gauauugugacauaucucugcacucaucacacaaaagaugcaaaucgccaugaggauccacagggagggggcauuuuaacuaccaccugaacuaaucauc

SEQ ID NO: 1442 - RNA, 100 nt, Artificial Sequence
  Description of Artificial Sequence Synthetic polynucleotide
gauauugugacauaucucugcacucaucacacaaaagaugcaaaucgccaugaggauccacagggagggggcauuucaauuacuggcugaacuaaucauc

SEQ ID NO: 1443 - RNA, 100 nt, Artificial Sequence
  Description of Artificial Sequence Synthetic polynucleotide
gauauugugacauaucucugcacucaucacacaaaagaugcaaaucguaggaaggauccacagggagggggcauucuaaucuugaccugaacuaaucauc

SEQ ID NO: 1444 - RNA, 100 nt, Artificial Sequence
  Description of Artificial Sequence Synthetic polynucleotide
gauauugugacauaucucugcacucaucacacaaaagaugcaaaucaagaacaggauccacagggagggggcaucuuaauuuugagcugaacuaaucauc

SEQ ID NO: 1445 - RNA, 100 nt, Artificial Sequence
  Description of Artificial Sequence Synthetic polynucleotide
gauauugugacauaucucugcacucaucacacaaaagaugcaaaucccgggaaggauccacagggagggggcauuuuaaucggcgacugaacuaaucauc

SEQ ID NO: 1446 - RNA, 100 nt, Artificial Sequence
  Description of Artificial Sequence Synthetic polynucleotide
gauauugugacauaucucugcacucaucacacaaaagaugcaaaugcagauuaggauccacagggagggggcauuuuaaccaccgacugaacuaaucauc

SEQ ID NO: 1447 - RNA, 100 nt, Artificial Sequence
  Description of Artificial Sequence Synthetic polynucleotide
gauauugugacauaucucugcacucaucacacaaaagaugcaaaugcagucuaggauccacagggagggggcauuucaauuggcagcugaacuaaucauc

SEQ ID NO: 1448 - RNA, 100 nt, Artificial Sequence
  Description of Artificial Sequence Synthetic polynucleotide
gauauugugacauaucucugcacucaucacacaaaagaugcaaauacaaccuaggauccacagggagggggcauucuaauuaaagacugaacuaaucauc

SEQ ID NO: 1449 - RNA, 100 nt, Artificial Sequence
  Description of Artificial Sequence Synthetic polynucleotide
gauauugugacauaucucugcacucaucacacaaaagaugcaaauacagauuaggauccacagggagggggcaucuuaauucaaaccugaacuaaucauc

SEQ ID NO: 1450 - RNA, 100 nt, Artificial Sequence
  Description of Artificial Sequence Synthetic polynucleotide
gauauugugacauaucucugcacucaucacacaaaagaugcaaauauagguuaggauccacagggagggggcauuuuaauucgaggcugaacuaaucauc

SEQ ID NO: 1451 - RNA, 100 nt, Artificial Sequence
  Description of Artificial Sequence Synthetic polynucleotide
gauauugugacauaucucugcacucaucacacaaaagaugcaaaaaccccauaggauccacagggagggggcauuuuaacccucagcugaacuaaucauc

SEQ ID NO: 1452 - RNA, 100 nt, Artificial Sequence
  Description of Artificial Sequence Synthetic polynucleotide
gauauugugacauaucucugcacucaucacacaaaagaugcaaagucaaaauaggauccacagggagggggcauuucaauccagaccugaacuaaucauc

SEQ ID NO: 1453 - RNA, 100 nt, Artificial Sequence
  Description of Artificial Sequence Synthetic polynucleotide
gauauugugacauaucucugcacucaucacacaaaagaugcaaaauuaaaauaggauccacagggagggggcauucuaaucaacggcugaacuaaucauc

SEQ ID NO: 1454 - RNA, 100 nt, Artificial Sequence
  Description of Artificial Sequence Synthetic polynucleotide
gauauugugacauaucucugcacucaucacacaaaagaugcaaacccguaauaggauccacagggagggggcaucuuaaucacgaccugaacuaaucauc

SEQ ID NO: 1455 - RNA, 100 nt, Artificial Sequence
  Description of Artificial Sequence Synthetic polynucleotide
gauauugugacauaucucugcacucaucacacaaaagaugcaaaagauaaauaggauccacagggagggggcauuuuaauucagaccugaacuaaucauc

SEQ ID NO: 1456 - RNA, 100 nt, Artificial Sequence
  Description of Artificial Sequence Synthetic polynucleotide
gauauugugacauaucucugcacucaucacacaaaagaugcaacgacaauauaggauccacagggagggggcauuuuaacacuuaacugaacuaaucauc

SEQ ID NO: 1457 - RNA, 100 nt, Artificial Sequence
  Description of Artificial Sequence Synthetic polynucleotide
gauauugugacauaucucugcacucaucacacaaaagaugcaacguuaauauaggauccacagggagggggcauuucaauccaaaccugaacuaaucauc

SEQ ID NO: 1458 - RNA, 100 nt, Artificial Sequence
  Description of Artificial Sequence Synthetic polynucleotide
gauauugugacauaucucugcacucaucacacaaaagaugcaacagugguauaggauccacagggagggggcauucuaauugcgaccugaacuaaucauc

SEQ ID NO: 1459 - RNA, 100 nt, Artificial Sequence
  Description of Artificial Sequence Synthetic polynucleotide
gauauugugacauaucucugcacucaucacacaaaagaugcaauagggauauaggauccacagggagggggcaucuuaaucauuaacugaacuaaucauc

SEQ ID NO: 1460 - RNA, 100 nt, Artificial Sequence
  Description of Artificial Sequence Synthetic polynucleotide
gauauugugacauaucucugcacucaucacacaaaagaugcaacaggaguauaggauccacagggagggggcauuuuaauugccaacugaacuaaucauc

SEQ ID NO: 1461 - RNA, 100 nt, Artificial Sequence
  Description of Artificial Sequence Synthetic polynucleotide
gauauugugacauaucucugcacucaucacacaaaagaugcacugggccuauaggauccacagggagggggcauuuuaacaaccggcugaacuaaucauc

SEQ ID NO: 1462 - RNA, 100 nt, Artificial Sequence
  Description of Artificial Sequence Synthetic polynucleotide
gauauugugacauaucucugcacucaucacacaaaagaugcauacgggcuauaggauccacagggagggggcauuucaauugugggcugaacuaaucauc

SEQ ID NO: 1463 - RNA, 100 nt, Artificial Sequence
  Description of Artificial Sequence Synthetic polynucleotide
gauauugugacauaucucugcacucaucacacaaaagaugcacaggggcuauaggauccacagggagggggcauucuaauucccugcugaacuaaucauc

SEQ ID NO: 1464 - RNA, 100 nt, Artificial Sequence
  Description of Artificial Sequence Synthetic polynucleotide
gauauugugacauaucucugcacucaucacacaaaagaugcauugagacuauaggauccacagggagggggcaucuuaauugggagcugaacuaaucauc

SEQ ID NO: 1465 - RNA, 100 nt, Artificial Sequence
  Description of Artificial Sequence Synthetic polynucleotide
gauauugugacauaucucugcacucaucacacaaaagaugcaucggcccuauaggauccacagggagggggcauuuuaaucauuaccugaacuaaucauc

SEQ ID NO: 1466 - RNA, 100 nt, Artificial Sequence
  Description of Artificial Sequence Synthetic polynucleotide
gauauugugacauaucucugcacucaucacacaaaagaugcucaaggucuauaggauccacagggagggggcauuuuaacuaccggcugaacuaaucauc

SEQ ID NO: 1467 - RNA, 100 nt, Artificial Sequence
  Description of Artificial Sequence Synthetic polynucleotide
gauauugugacauaucucugcacucaucacacaaaagaugcuuucacucuauaggauccacagggagggggcauuucaauuuuggacugaacuaaucauc

SEQ ID NO: 1468 - RNA, 100 nt, Artificial Sequence
  Description of Artificial Sequence Synthetic polynucleotide
gauauugugacauaucucugcacucaucacacaaaagaugcuuuggcucuauaggauccacagggagggggcauucuaaucuuggacugaacuaaucauc

SEQ ID NO: 1469 - RNA, 100 nt, Artificial Sequence
  Description of Artificial Sequence Synthetic polynucleotide
gauauugugacauaucucugcacucaucacacaaaagaugccucagaucuauaggauccacagggagggggcaucuuaauccagggcugaacuaaucauc

SEQ ID NO: 1470 - RNA, 100 nt, Artificial Sequence
  Description of Artificial Sequence Synthetic polynucleotide
gauauugugacauaucucugcacucaucacacaaaagaugccauaaaucuauaggauccacagggagggggcauuuuaaucacuagcugaacuaaucauc

SEQ ID NO: 1471 - RNA, 100 nt, Artificial Sequence
  Description of Artificial Sequence Synthetic polynucleotide
gauauugugacauaucucugcacucaucacacaaaagaugagccaguucuauaggauccacagggagggggcauuuuaacuuugugcugaacuaaucauc

SEQ ID NO: 1472 - RNA, 100 nt, Artificial Sequence
  Description of Artificial Sequence Synthetic polynucleotide
gauauugugacauaucucugcacucaucacacaaaagaugaaacuauucuauaggauccacagggagggggcauuucaaucaggugcugaacuaaucauc

SEQ ID NO: 1473 - RNA, 100 nt, Artificial Sequence
  Description of Artificial Sequence Synthetic polynucleotide
gauauugugacauaucucugcacucaucacacaaaagauggcucaauucuauaggauccacagggagggggcauucuaaucacugacugaacuaaucauc

SEQ ID NO: 1474 - RNA, 100 nt, Artificial Sequence
  Description of Artificial Sequence Synthetic polynucleotide
gauauugugacauaucucugcacucaucacacaaaagauggcgccauucuauaggauccacagggagggggcaucuuaaucacaaacugaacuaaucauc

SEQ ID NO: 1475 - RNA, 100 nt, Artificial Sequence
  Description of Artificial Sequence Synthetic polynucleotide
gauauugugacauaucucugcacucaucacacaaaagaugauucgguucuauaggauccacagggagggggcauuuuaauucugggcugaacuaaucauc

SEQ ID NO: 1476 - RNA, 100 nt, Artificial Sequence
  Description of Artificial Sequence Synthetic polynucleotide
gauauugugacauaucucugcacucaucacacaaaagauuacugacuucuauaggauccacagggagggggcauuuuaacgagcggcugaacuaaucauc

SEQ ID NO: 1477 - RNA, 100 nt, Artificial Sequence
  Description of Artificial Sequence Synthetic polynucleotide
gauauugugacauaucucugcacucaucacacaaaagauuaauaacuucuauaggauccacagggagggggcauuucaauuaccaacugaacuaaucauc

SEQ ID NO: 1478 - RNA, 100 nt, Artificial Sequence
  Description of Artificial Sequence Synthetic polynucleotide
gauauugugacauaucucugcacucaucacacaaaagaucuucaccuucuauaggauccacagggagggggcauucuaauugcccacugaacuaaucauc

SEQ ID NO: 1479 - RNA, 100 nt, Artificial Sequence
  Description of Artificial Sequence Synthetic polynucleotide
gauauugugacauaucucugcacucaucacacaaaagaucuugcccuucuauaggauccacagggagggggcaucuuaauuaccggcugaacuaaucauc

SEQ ID NO: 1480 - RNA, 100 nt, Artificial Sequence
  Description of Artificial Sequence Synthetic polynucleotide
gauauugugacauaucucugcacucaucacacaaaagaucuuuaccuucuauaggauccacagggagggggcauuuuaaucgaaaacugaacuaaucauc

SEQ ID NO: 1481 - RNA, 100 nt, Artificial Sequence
  Description of Artificial Sequence Synthetic polynucleotide
gauauugugacauaucucugcacucaucacacaaaagacaguucucuucuauaggauccacagggagggggcauuuuaacacuuggcugaacuaaucauc

SEQ ID NO: 1482 - RNA, 100 nt, Artificial Sequence
  Description of Artificial Sequence Synthetic polynucleotide
gauauugugacauaucucugcacucaucacacaaaagacaaucuucuucuauaggauccacagggagggggcauuucaauuggcgacugaacuaaucauc

SEQ ID NO: 1483 - RNA, 100 nt, Artificial Sequence
  Description of Artificial Sequence Synthetic polynucleotide
gauauugugacauaucucugcacucaucacacaaaagacccccuucuucuauaggauccacagggagggggcauucuaaucguuaacugaacuaaucauc

SEQ ID NO: 1484 - RNA, 100 nt, Artificial Sequence
  Description of Artificial Sequence Synthetic polynucleotide
gauauugugacauaucucugcacucaucacacaaaagaaagguuucuucuauaggauccacagggagggggcaucuuaauugugggcugaacuaaucauc

SEQ ID NO: 1485 - RNA, 100 nt, Artificial Sequence
  Description of Artificial Sequence Synthetic polynucleotide
gauauugugacauaucucugcacucaucacacaaaagagaccacucuucuauaggauccacagggagggggcauuuuaauuuuaagcugaacuaaucauc

SEQ ID NO: 1486 - RNA, 100 nt, Artificial Sequence
  Description of Artificial Sequence Synthetic polynucleotide
gauauugugacauaucucugcacucaucacacaaaagccagauaucuucuauaggauccacagggagggggcauuuuaacugcuagcugaacuaaucauc

SEQ ID NO: 1487 - RNA, 100 nt, Artificial Sequence
  Description of Artificial Sequence Synthetic polynucleotide
gauauugugacauaucucugcacucaucacacaaaagucuuuuaucuucuauaggauccacagggagggggcauuucaauugcagacugaacuaaucauc

SEQ ID NO: 1488 - RNA, 100 nt, Artificial Sequence
  Description of Artificial Sequence Synthetic polynucleotide
gauauugugacauaucucugcacucaucacacaaaagcuuucuaucuucuauaggauccacagggagggggcauucuaaucgacggcugaacuaaucauc

SEQ ID NO: 1489 - RNA, 100 nt, Artificial Sequence
  Description of Artificial Sequence Synthetic polynucleotide
gauauugugacauaucucugcacucaucacacaaaagucaucuaucuucuauaggauccacagggagggggcaucuuaauuauaggcugaacuaaucauc

SEQ ID NO: 1490 - RNA, 100 nt, Artificial Sequence
  Description of Artificial Sequence Synthetic polynucleotide
gauauugugacauaucucugcacucaucacacaaaagcauaguaucuucuauaggauccacagggagggggcauuuuaaucaccugcugaacuaaucauc

SEQ ID NO: 1491 - RNA, 100 nt, Artificial Sequence
  Description of Artificial Sequence Synthetic polynucleotide
gauauugugacauaucucugcacucaucacacaaaacagaauaaucuucuauaggauccacagggagggggcauuuuaacucuaagcugaacuaaucauc

SEQ ID NO: 1492 - RNA, 100 nt, Artificial Sequence
  Description of Artificial Sequence Synthetic polynucleotide
gauauugugacauaucucugcacucaucacacaaaaauuuacaaucuucuauaggauccacagggagggggcauuucaauucgagacugaacuaaucauc

SEQ ID NO: 1493 - RNA, 100 nt, Artificial Sequence
  Description of Artificial Sequence Synthetic polynucleotide
gauauugugacauaucucugcacucaucacacaaaaccuaucaaucuucuauaggauccacagggagggggcauucuaaucuccggcugaacuaaucauc

SEQ ID NO: 1494 - RNA, 100 nt, Artificial Sequence
  Description of Artificial Sequence Synthetic polynucleotide
gauauugugacauaucucugcacucaucacacaaaaucucccaaucuucuauaggauccacagggagggggcauuuuaauuggacgcugaacuaaucauc

SEQ ID NO: 1495 - RNA, 100 nt, Artificial Sequence
  Description of Artificial Sequence Synthetic polynucleotide
gauauugugacauaucucugcacucaucacacaaauucuaaaaaucuucuauaggauccacagggagggggcauuuuaaccguuaacugaacuaaucauc

SEQ ID NO: 1496 - RNA, 100 nt, Artificial Sequence
  Description of Artificial Sequence Synthetic polynucleotide
gauauugugacauaucucugcacucaucacacaaacucuaaaaaucuucuauaggauccacagggagggggcauuucaaucauuggcugaacuaaucauc

SEQ ID NO: 1497 - RNA, 100 nt, Artificial Sequence
  Description of Artificial Sequence Synthetic polynucleotide
gauauugugacauaucucugcacucaucacacaaauacgagaaaucuucuauaggauccacagggagggggcauucuaauugaccacugaacuaaucauc

SEQ ID NO: 1498 - RNA, 100 nt, Artificial Sequence
  Description of Artificial Sequence Synthetic polynucleotide
gauauugugacauaucucugcacucaucacacaaauccggaaaaucuucuauaggauccacagggagggggcaucuuaauucggggcugaacuaaucauc

SEQ ID NO: 1499 - RNA, 100 nt, Artificial Sequence
  Description of Artificial Sequence Synthetic polynucleotide
gauauugugacauaucucugcacucaucacacaaauuaacaaaaucuucuauaggauccacagggagggggcauuuuaauuccuggcugaacuaaucauc

SEQ ID NO: 1500 - RNA, 100 nt, Artificial Sequence
  Description of Artificial Sequence Synthetic polynucleotide
gauauugugacauaucucugcacucaucacacaauauagccaaaucuucuauaggauccacagggagggggcauuuuaacacaggacugaacuaaucauc

SEQ ID NO: 1501 - RNA, 100 nt, Artificial Sequence
  Description of Artificial Sequence Synthetic polynucleotide
gauauugugacauaucucugcacucaucacacaaugcagccaaaucuucuauaggauccacagggagggggcauuucaaucagguacugaacuaaucauc

SEQ ID NO: 1502 - RNA, 100 nt, Artificial Sequence
  Description of Artificial Sequence Synthetic polynucleotide
gauauugugacauaucucugcacucaucacacaaccacaacaaaucuucuauaggauccacagggagggggcauucuaauuagggacugaacuaaucauc

SEQ ID NO: 1503 - RNA, 100 nt, Artificial Sequence
  Description of Artificial Sequence Synthetic polynucleotide
gauauugugacauaucucugcacucaucacacaauuuacucaaaucuucuauaggauccacagggagggggcaucuuaaucuccgacugaacuaaucauc

SEQ ID NO: 1504 - RNA, 100 nt, Artificial Sequence
  Description of Artificial Sequence Synthetic polynucleotide
gauauugugacauaucucugcacucaucacacaacacuuacaaaucuucuauaggauccacagggagggggcauuuuaauuccaugcugaacuaaucauc

SEQ ID NO: 1505 - RNA, 100 nt, Artificial Sequence
  Description of Artificial Sequence Synthetic polynucleotide
gauauugugacauaucucugcacucaucacacauaugcggcaaaucuucuauaggauccacagggagggggcauuuuaaccuuagacugaacuaaucauc

SEQ ID NO: 1506 - RNA, 100 nt, Artificial Sequence
  Description of Artificial Sequence Synthetic polynucleotide
gauauugugacauaucucugcacucaucacacauacaaagcaaaucuucuauaggauccacagggagggggcauuucaaucccugacugaacuaaucauc

SEQ ID NO: 1507 - RNA, 100 nt, Artificial Sequence
  Description of Artificial Sequence Synthetic polynucleotide
gauauugugacauaucucugcacucaucacacacuuaacgcaaaucuucuauaggauccacagggagggggcauucuaauccagggcugaacuaaucauc

SEQ ID NO: 1508 - RNA, 100 nt, Artificial Sequence
  Description of Artificial Sequence Synthetic polynucleotide
gauauugugacauaucucugcacucaucacacacgcccggcaaaucuucuauaggauccacagggagggggcaucuuaaucaccagcugaacuaaucauc

SEQ ID NO: 1509 - RNA, 100 nt, Artificial Sequence
  Description of Artificial Sequence Synthetic polynucleotide
gauauugugacauaucucugcacucaucacacauuuuccgcaaaucuucuauaggauccacagggagggggcauuuuaaucgaguacugaacuaaucauc

SEQ ID NO: 1510 - RNA, 100 nt, Artificial Sequence
  Description of Artificial Sequence Synthetic polynucleotide
gauauugugacauaucucugcacucaucacacuucacuugcaaaucuucuauaggauccacagggagggggcauuuuaacccuccacugaacuaaucauc

SEQ ID NO: 1511 - RNA, 100 nt, Artificial Sequence
  Description of Artificial Sequence Synthetic polynucleotide
gauauugugacauaucucugcacucaucacacuucgcuugcaaaucuucuauaggauccacagggagggggcauuucaaucguuaccugaacuaaucauc

SEQ ID NO: 1512 - RNA, 100 nt, Artificial Sequence
  Description of Artificial Sequence Synthetic polynucleotide
gauauugugacauaucucugcacucaucacacuauaacugcaaaucuucuauaggauccacagggagggggcauucuaauucuggacugaacuaaucauc

SEQ ID NO: 1513 - RNA, 100 nt, Artificial Sequence
  Description of Artificial Sequence Synthetic polynucleotide
gauauugugacauaucucugcacucaucacaccuugucugcaaaucuucuauaggauccacagggagggggcaucuuaauuagggacugaacuaaucauc

SEQ ID NO: 1514 - RNA, 100 nt, Artificial Sequence
  Description of Artificial Sequence Synthetic polynucleotide
gauauugugacauaucucugcacucaucacacucucguugcaaaucuucuauaggauccacagggagggggcauuuuaauuaagugcugaacuaaucauc

SEQ ID NO: 1515 - RNA, 100 nt, Artificial Sequence
  Description of Artificial Sequence Synthetic polynucleotide
gauauugugacauaucucugcacucaucacagugcuaaugcaaaucuucuauaggauccacagggagggggcauuuuaacacgcggcugaacuaaucauc

SEQ ID NO: 1516 - RNA, 100 nt, Artificial Sequence
  Description of Artificial Sequence Synthetic polynucleotide
gauauugugacauaucucugcacucaucacaagcuauaugcaaaucuucuauaggauccacagggagggggcauuucaauccaaaccugaacuaaucauc

SEQ ID NO: 1517 - RNA, 100 nt, Artificial Sequence
  Description of Artificial Sequence Synthetic polynucleotide
gauauugugacauaucucugcacucaucacagcccccaugcaaaucuucuauaggauccacagggagggggcauucuaauccgacgcugaacuaaucauc

SEQ ID NO: 1518 - RNA, 100 nt, Artificial Sequence
  Description of Artificial Sequence Synthetic polynucleotide
gauauugugacauaucucugcacucaucacagugcucaugcaaaucuucuauaggauccacagggagggggcaucuuaauugaaaccugaacuaaucauc

SEQ ID NO: 1519 - RNA, 100 nt, Artificial Sequence
  Description of Artificial Sequence Synthetic polynucleotide
gauauugugacauaucucugcacucaucacaauuuguaugcaaaucuucuauaggauccacagggagggggcauuuuaauccuagacugaacuaaucauc

SEQ ID NO: 1520 - RNA, 100 nt, Artificial Sequence
  Description of Artificial Sequence Synthetic polynucleotide
gauauugugacauaucucugcacucaucacuuuacugaugcaaaucuucuauaggauccacagggagggggcauuuuaacucuuaacugaacuaaucauc

SEQ ID NO: 1521 - RNA, 100 nt, Artificial Sequence
  Description of Artificial Sequence Synthetic polynucleotide
gauauugugacauaucucugcacucaucacuuuagugaugcaaaucuucuauaggauccacagggagggggcauuucaauugcguacugaacuaaucauc

SEQ ID NO: 1522 - RNA, 100 nt, Artificial Sequence
  Description of Artificial Sequence Synthetic polynucleotide
gauauugugacauaucucugcacucaucacuagcucgaugcaaaucuucuauaggauccacagggagggggcauucuaaucuuuaccugaacuaaucauc

SEQ ID NO: 1523 - RNA, 100 nt, Artificial Sequence
  Description of Artificial Sequence Synthetic polynucleotide
gauauugugacauaucucugcacucaucaccuuuccgaugcaaaucuucuauaggauccacagggagggggcauuuuaauuuuagacugaacuaaucauc

SEQ ID NO: 1524 - RNA, 100 nt, Artificial Sequence
  Description of Artificial Sequence Synthetic polynucleotide
gauauugugaauuaucucuggccucaucacuaaaaagaugacaaucuucuuaaggauccaacgggaggggagauuuuaauccaucucugauguaaucauc

SEQ ID NO: 1525 - RNA, 100 nt, Artificial Sequence
  Description of Artificial Sequence Synthetic polynucleotide
gauauugugacaacgcucugcacgauucacacaacaaaugcaaauaugcuauaggaagaacagggagaaagcauuuuauuuuaucucugccguaaucauc

SEQ ID NO: 1526 - RNA, 100 nt, Artificial Sequence
  Description of Artificial Sequence Synthetic polynucleotide
gauaauacgacauaucgaaccacucaucuaugaaaagaugaaccucuucuaucaccuccacaggccuugggcauuucuucauaucucucuuguaaucauc

SEQ ID NO: 1527 - RNA, 100 nt, Artificial Sequence
  Description of Artificial Sequence Synthetic polynucleotide
gauauugugcgaauucucugcagauuccacacaaacaagccaaaucuugauucggauccaccacaugggggcauacuucuauaucucauucguaaucauc

SEQ ID NO: 1528 - RNA, 100 nt, Artificial Sequence
  Description of Artificial Sequence Synthetic polynucleotide
gaccuggggacauaucgagagucucaucaccucuaugaugcaaaacacggauaggaucaucucagagggggcuuuaacauauaucuauaauguaaucauc

SEQ ID NO: 1529 - RNA, 100 nt, Artificial Sequence
  Description of Artificial Sequence Synthetic polynucleotide
gauauugugaagcuggacugcacuccaaagugaaaagaugguacuacucuauagguaauugugggaggggauaauacaauauaucggucauguaaucauc

SEQ ID NO: 1530 - RNA, 100 nt, Artificial Sequence
  Description of Artificial Sequence Synthetic polynucleotide
gauacuauuaauuaucucugguggauggacacaaaaacuccgucucuucuaucuaauagucagggaggcgauuuaguaauauaugcguccaauaaucauc

SEQ ID NO: 1531 - RNA, 100 nt, Artificial Sequence
  Description of Artificial Sequence Synthetic polynucleotide
gauacggugacauauccgugcacucauccaacaaaagaugguaaucuucuauucgauccacagguugggggcauuuguauauaucucuacacuaaucauc

SEQ ID NO: 1532 - RNA, 100 nt, Artificial Sequence
  Description of Artificial Sequence Synthetic polynucleotide
gauauuguguguuaucucugcagaaaucacacaaaccuugcaaaucuuagcuaggauccacuaugagggggcauaccaauauaucucaauacuaaucauc

SEQ ID NO: 1533 - RNA, 100 nt, Artificial Sequence
  Description of Artificial Sequence Synthetic polynucleotide
gaguacgugacauauccacccacucaucaccuccaagaugcaaaguaacuauaggaucaucugggagggggcugaauaauauaucugaucacuaaucauc

SEQ ID NO: 1534 - RNA, 100 nt, Artificial Sequence
  Description of Artificial Sequence Synthetic polynucleotide
gauauugugaguacacucugcacucccaaaacaaaagauggucaccuucuauaggcuagucagggagggguaaaauuaauauaucaaccuacuaaucauc

SEQ ID NO: 1535 - RNA, 100 nt, Artificial Sequence
  Description of Artificial Sequence Synthetic polynucleotide
gauacuuuaucauaucucugguuggcucacacaaaauacaguaaucuucuauccuacgcacagggaggccuaccuuuaauauauaagcacacuaaucauc

SEQ ID NO: 1536 - RNA, 100 nt, Artificial Sequence
  Description of Artificial Sequence Synthetic polynucleotide
gauauugugacauaugcauaaucucaucacacuucauucgcaaaucuucgcgaugcuccacagggauuucccuuuuuaauauacacccccacuaaucauc

SEQ ID NO: 1537 - RNA, 100 nt, Artificial Sequence
  Description of Artificial Sequence Synthetic polynucleotide
gauauugugagcugcaaaugcacucaucugugacccgaugcaaauccuacacaagauccacaggcauugcugauuuuaauauuugggcguacuaaucauc

SEQ ID NO: 1538 - RNA, 100 nt, Artificial Sequence
  Description of Artificial Sequence Synthetic polynucleotide
gaccuugugacauaucgaugcacucaucaccgaaaagaugcaaacauucuauaggaucaucagggagggggcucuuuaauauaucugagaacuaaucauc

SEQ ID NO: 1539 - RNA, 100 nt, Artificial Sequence
  Description of Artificial Sequence Synthetic polynucleotide
gauauugugaaccaucucugcacuccggacacaaaagaugguuaucuucuauagguagcacagggaggggaacuuuuaauauauccaggaacuaaucauc

SEQ ID NO: 1540 - RNA, 100 nt, Artificial Sequence
  Description of Artificial Sequence Synthetic polynucleotide
gauaacaagacauaucucugguagcaucacacaaaacucccaaaucuucuaucccuuccacagggaggcucgauuuuaauauauauaagaacuaaucauc

SEQ ID NO: 1541 - RNA, 100 nt, Artificial Sequence
  Description of Artificial Sequence Synthetic polynucleotide
gauauugugacauaugaauucacucaucacacuuuaaaugcaaaucuucacucugauccacagggaucacucauuuuaauauacaggcgaacuaaucauc

SEQ ID NO: 1542 - RNA, 100 nt, Artificial Sequence
  Description of Artificial Sequence Synthetic polynucleotide
gauauugugaguuccgucugcacucaucugaaauaagaugcaaaucgcagagaggauccacaggcccacagcauuuuaauaucagcccgaacuaaucauc

SEQ ID NO: 1543 - RNA, 100 nt, Artificial Sequence
  Description of Artificial Sequence Synthetic polynucleotide
gauauccgaaguuaucucugcacuacccccucaaaagaugcaacaaauggauaggauccacaccucgacggcauuuuaauaaaggccggaacuaaucauc

SEQ ID NO: 1544 - RNA, 100 nt, Artificial Sequence
  Description of Artificial Sequence Synthetic polynucleotide
gauauugugacauaucucugaaggguagacacaaaagaugaccuguaccuauaggauccaguauacaagggcauuuuaauugugauaagaacuaaucauc

SEQ ID NO: 1545 - RNA, 100 nt, Artificial Sequence
  Description of Artificial Sequence Synthetic polynucleotide
gauaaggugacauaucucuggucucaucacacaaaaccugcaaaucuucuauuugauccacagggaggaugcauuuuaauauaugccugaacuaaucauc

SEQ ID NO: 1546 - RNA, 100 nt, Artificial Sequence
  Description of Artificial Sequence Synthetic polynucleotide
gauauugugacauauauaugcacucaucacaccuuagaugcaaaucuucccaaggauccacagggauaaggcauuuuaauauaggacugaacuaaucauc

SEQ ID NO: 1547 - RNA, 100 nt, Artificial Sequence
  Description of Artificial Sequence Synthetic polynucleotide
gauauugugagccuucucugcacucaucugugaaaagaugcaaauccggcauaggauccacaggcaccgggcauuuuaauauuaggcugaacuaaucauc

SEQ ID NO: 1548 - RNA, 100 nt, Artificial Sequence
  Description of Artificial Sequence Synthetic polynucleotide
gauaugcuuccauaucucugcacuauagucacaaaagaugcaauaauccuauaggauccacauuaccggggcauuuuaauaaugaccugaacuaaucauc

SEQ ID NO: 1549 - RNA, 100 nt, Artificial Sequence
  Description of Artificial Sequence Synthetic polynucleotide
gauauugugacauaucucugauguguucacacaaaagaugaacucguucuauaggauccaauuacugggggcauuuuaauuaagaccugaacuaaucauc

SEQ ID NO: 1550 - RNA, 100 nt, Artificial Sequence
  Description of Artificial Sequence Synthetic polynucleotide
gauauugugacauaucggucgaaucaucacacaaaagcgaguuuucuucuauaggaucauuaaaaagggggcauuuuaagauuuaccugaacuaaucauc

SEQ ID NO: 1551 - RNA, 100 nt, Artificial Sequence
  Description of Artificial Sequence Synthetic polynucleotide
gauauugugacaaccacgaccacucaucacacaauaaaaacuaaucuucuauaggaaaguaucagagggggcauuuuaugcgcuugcugaacuaaucauc

SEQ ID NO: 1552 - RNA, 100 nt, Artificial Sequence
  Description of Artificial Sequence Synthetic polynucleotide
gauauugugagcuaucucugcacucauccgacaaaagaugcaaaucggcuauaggauccacagguugggggcauuuuaauaucacucugaacuaaucauc

SEQ ID NO: 1553 - RNA, 100 nt, Artificial Sequence
  Description of Artificial Sequence Synthetic polynucleotide
gauauccggacauaucucugcacugugcacacaaaagaugcaaugguucuauaggauccacaaccagggggcauuuuaauagcccucugaacuaaucauc

SEQ ID NO: 1554 - RNA, 100 nt, Artificial Sequence
  Description of Artificial Sequence Synthetic polynucleotide
gauauugugacauaucucugguaacaucacacaaaagaugauucucuucuauaggauccaaaucgagggggcauuuuaauccaacucugaacuaaucauc

SEQ ID NO: 1555 - RNA, 100 nt, Artificial Sequence
  Description of Artificial Sequence Synthetic polynucleotide
gauauugugacauaucgaccaacucaucacacaaaaguccguaaucuucuauaggaucguccuggagggggcauuuuaacugaacucugaacuaaucauc

SEQ ID NO: 1556 - RNA, 100 nt, Artificial Sequence
  Description of Artificial Sequence Synthetic polynucleotide
gauauugugacagugaagugcacucaucacacaauuaaaucaaaucuucuauaggaaagaaugggagggggcauuuuauaagaccucugaacuaaucauc

SEQ ID NO: 1557 - RNA, 100 nt, Artificial Sequence
  Description of Artificial Sequence Synthetic polynucleotide
gauauuguuuuucaccucugcacucaucacaguucaacugcaaaucuucuauagcagaaaaagggagggggcauuuucuauuuacucugaacuaaucauc

SEQ ID NO: 1558 - RNA, 100 nt, Artificial Sequence
  Description of Artificial Sequence Synthetic polynucleotide
gauacgagaaguuaucucugcacucauccuugauccgaugcaaaucuucuauccguuagucagggagggggcauuugccuaaaacucugaacuaaucauc

SEQ ID NO: 1559 - RNA, 100 nt, Artificial Sequence
  Description of Artificial Sequence Synthetic polynucleotide
gauauugugacagcucucugcacucaucacacaaucgaugcaaaucuucuauaggauacacagggagggggcauuuuaugauaucucugaacuaaucauc

SEQ ID NO: 1560 - RNA, 100 nt, Artificial Sequence
  Description of Artificial Sequence Synthetic polynucleotide
gauauuguccgauaucucugcacucaucacaaacaagaugcaaaucuucuauaguucccacagggagggggcauuuuucgauaucucugaacuaaucauc

SEQ ID NO: 1561 - RNA, 100 nt, Artificial Sequence
  Description of Artificial Sequence Synthetic polynucleotide
gauaggccgacauaucucugcacucaucugugaaaagaugcaaaucuucuaucuacuccacagggagggggcauuuguagauaucucugaacuaaucauc

SEQ ID NO: 1562 - RNA, 100 nt, Artificial Sequence
  Description of Artificial Sequence Synthetic polynucleotide
gauauugugacauaucucugcacucccgugacaaaagaugcaaaucuucuuucauauccacagggagggggcauucguucauaucucugaacuaaucauc

SEQ ID NO: 1563 - RNA, 100 nt, Artificial Sequence
  Description of Artificial Sequence Synthetic polynucleotide
gauauugugacauaucucugcagcgcuaacacaaaagaugcaaaucuuggagccgauccacagggagggggcaucgucacauaucucugaacuaaucauc

SEQ ID NO: 1564 - RNA, 100 nt, Artificial Sequence
  Description of Artificial Sequence Synthetic polynucleotide
gauauugugacauaucucuaaagaauucacacaaaagaugcaaaucauauuuuggauccacagggagggggcagauucuaauaucucugaacuaaucauc

SEQ ID NO: 1565 - RNA, 100 nt, Artificial Sequence
  Description of Artificial Sequence Synthetic polynucleotide
gauauugugacauaucgagagugacaucacacaaaagaugcaaacgguauuaaggauccacagggagggggccuauaaccauaucucugaacuaaucauc

SEQ ID NO: 1566 - RNA, 100 nt, Artificial Sequence
  Description of Artificial Sequence Synthetic polynucleotide
gccauugugugauaucugagcacuccgcacacaccagaugccugaccucuauaggauccacagggagggggcauuucaauauaucggaacucuaaucauc

SEQ ID NO: 1567 - RNA, 100 nt, Artificial Sequence
  Description of Artificial Sequence Synthetic polynucleotide
gauaugcagacauagaccugcaccuuucacacuauagaugccugaccucuauaggauccacagggagggggcauuucaauauaucggaacucuaaucauc

SEQ ID NO: 1568 - RNA, 100 nt, Artificial Sequence
  Description of Artificial Sequence Synthetic polynucleotide
guucgugugaccaaacucugccaagaucacaguauagaugccugaccucuauaggauccacagggagggggcauuucaauauaucggaacucuaaucauc

SEQ ID NO: 1569 - RNA, 100 nt, Artificial Sequence
  Description of Artificial Sequence Synthetic polynucleotide
gauauuguccaaaaucucucuuuccaucacuguucagaugccugaccucuauaggauccacagggagggggcauuucaauauaucggaacucuaaucauc

SEQ ID NO: 1570 - RNA, 100 nt, Artificial Sequence
  Description of Artificial Sequence Synthetic polynucleotide
gugauugugacuaaucucugccgucaucacaauaaagaugccugaccucuauaggauccacagggagggggcauuucaauauaucggaacucuaaucauc

SEQ ID NO: 1571 - RNA, 100 nt, Artificial Sequence
  Description of Artificial Sequence Synthetic polynucleotide
gauauuguccaauaucucuaaucucaucacuguaaagaugccugaccucuauaggauccacagggagggggcauuucaauauaucggaacucuaaucauc

SEQ ID NO: 1572 - RNA, 100 nt, Artificial Sequence
  Description of Artificial Sequence Synthetic polynucleotide
gauaucuuuacauaucuggugacucaucaaccuaaagaugccugaccucuauaggauccacagggagggggcauuucaauauaucggaacucuaaucauc

SEQ ID NO: 1573 - RNA, 100 nt, Artificial Sequence
  Description of Artificial Sequence Synthetic polynucleotide
gaaagucugacauaugcacccacucaucugaauaaagaugccugaccucuauaggauccacagggagggggcauuucaauauaucggaacucuaaucauc

SEQ ID NO: 1574 - RNA, 100 nt, Artificial Sequence
  Description of Artificial Sequence Synthetic polynucleotide
gauauacugacauaucuaagcacucaucagccaaaagaugccugaccucuauaggauccacagggagggggcauuucaauauaucggaacucuaaucauc

SEQ ID NO: 1575 - RNA, 100 nt, Artificial Sequence
  Description of Artificial Sequence Synthetic polynucleotide
gagugugugacauaugagugcacucauccuucaaaagaugccugaccucuauaggauccacagggagggggcauuucaauauaucggaacucuaaucauc

SEQ ID NO: 1576 - RNA, 100 nt, Artificial Sequence
  Description of Artificial Sequence Synthetic polynucleotide
gauauugugacauuggccugcacucauguaucaaaagaugccugaccucuauaggauccacagggagggggcauuucaauauaucggaacucuaaucauc

SEQ ID NO: 1577 - RNA, 100 nt, Artificial Sequence
  Description of Artificial Sequence Synthetic polynucleotide
gauauugugaccuuaaucugcacucagccuucaaaagaugccugaccucuauaggauccacagggagggggcauuucaauauaucggaacucuaaucauc

SEQ ID NO: 1578 - RNA, 100 nt, Artificial Sequence
  Description of Artificial Sequence Synthetic polynucleotide
gauauugugacauugcucugcacucaugccacaaaagaugccugaccucuauaggauccacagggagggggcauuucaauauaucggaacucuaaucauc

SEQ ID NO: 1579 - RNA, 100 nt, Artificial Sequence
  Description of Artificial Sequence Synthetic polynucleotide
gauauugugacuacucucugcacucaagccacaaaagaugccugaccucuauaggauccacagggagggggcauuucaauauaucggaacucuaaucauc

SEQ ID NO: 1580 - RNA, 100 nt, Artificial Sequence
  Description of Artificial Sequence Synthetic polynucleotide
gauauugugugucaucucugcacuccuaccacaaaagaugccugaccucuauaggauccacagggagggggcauuucaauauaucggaacucuaaucauc

SEQ ID NO: 1581 - RNA, 100 nt, Artificial Sequence
  Description of Artificial Sequence Synthetic polynucleotide
gauauuggcaguuaucucugcacuguagucacaaaagaugccugaccucuauaggauccacagggagggggcauuucaauauaucggaacucuaaucauc

SEQ ID NO: 1582 - RNA, 100 nt, Artificial Sequence
  Description of Artificial Sequence Synthetic polynucleotide
gauauuguguaauaucucugcacucuacacacaaaagaugccugaccucuauaggauccacagggagggggcauuucaauauaucggaacucuaaucauc

SEQ ID NO: 1583 - RNA, 100 nt, Artificial Sequence
  Description of Artificial Sequence Synthetic polynucleotide
gauauugauucauaucucugcacugcgcacacaaaagaugccugaccucuauaggauccacagggagggggcauuucaauauaucggaacucuaaucauc

SEQ ID NO: 1584 - RNA, 100 nt, Artificial Sequence
  Description of Artificial Sequence Synthetic polynucleotide
gauauauaaacauaucucugcacagcccacacaaaagaugccugaccucuauaggauccacagggagggggcauuucaauauaucggaacucuaaucauc

SEQ ID NO: 1585 - RNA, 100 nt, Artificial Sequence
  Description of Artificial Sequence Synthetic polynucleotide
gauucaaggacauaucucugcagaaaacacacaaaagaugccugaccucuauaggauccacagggagggggcauuucaauauaucggaacucuaaucauc

SEQ ID NO: 1586 - RNA, 100 nt, Artificial Sequence
  Description of Artificial Sequence Synthetic polynucleotide
gauauccugacauaucucugcacaaaucacacaaaagaugccugaccucuauaggauccacagggagggggcauuucaauauaucggaacucuaaucauc

SEQ ID NO: 1587 - RNA, 100 nt, Artificial Sequence
  Description of Artificial Sequence Synthetic polynucleotide
gauucagugacauaucucugcagcgaucacacaaaagaugccugaccucuauaggauccacagggagggggcauuucaauauaucggaacucuaaucauc

SEQ ID NO: 1588 - RNA, 100 nt, Artificial Sequence
  Description of Artificial Sequence Synthetic polynucleotide
gcgcgugugacauaucucugccaaaaucacacaaaagaugccugaccucuauaggauccacagggagggggcauuucaauauaucggaacucuaaucauc

SEQ ID NO: 1589 - RNA, 100 nt, Artificial Sequence
  Description of Artificial Sequence Synthetic polynucleotide
gauauugugacauaucucugaugggaucacacaaaagaugccugaccucuauaggauccacagggagggggcauuucaauauaucggaacucuaaucauc

SEQ ID NO: 1590 - DNA, 32 bp, Artificial Sequence
  Description of Artificial Sequence Synthetic oligonucleotide
taggctttctggctttttaccggaaagcccct

SEQ ID NO: 1591 - RNA, 32 nt, Artificial Sequence
  Description of Artificial Sequence Synthetic oligonucleotide
uaggcuuucuggcuuuuuaccggaaagccccu

SEQ ID NO: 1592 - DNA, 31 bp, Artificial Sequence
  Description of Artificial Sequence Synthetic oligonucleotide
caggttttctgacttcggtcggaaaacccct

SEQ ID NO: 1593 - RNA, 31 nt, Artificial Sequence
  Description of Artificial Sequence Synthetic oligonucleotide
cagguuuucugacuucggucggaaaaccccu

SEQ ID NO: 1594 - DNA, 11 bp, Artificial Sequence
  Description of Artificial Sequence Synthetic oligonucleotide
aatttttggag

SEQ ID NO: 1595 - RNA, 11 nt, Artificial Sequence
  Description of Artificial Sequence Synthetic oligonucleotide
aauuuuuggag

SEQ ID NO: 1596 - DNA, 249 bp, Artificial Sequence
  Description of Artificial Sequence Synthetic polynucleotide
gagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 1597 - DNA, 341 bp, Artificial Sequence
  Description of Artificial Sequence Synthetic polynucleotide
gtactgagtcgcccagtctcagatagatccgacgccgccatctctaggcccgcgccggccccctcgcacagacttgtgggagaagctcggctactcccctgccccggttaatttgcatataatatttcctagtaactatagaggcttaatgtgcgataaaagacagataatctgttctttttaatactagctacattttacatgataggcttggatttctataagagatacaaatactaaattattattttaaaaaacagcacaaaaggaaactcaccctaactgtaaagtaattgtgtgttttgagactataaatatcccttggagaaaagccttgtttg

SEQ ID NO: 1598 - DNA, 263 bp, Artificial Sequence
  Description of Artificial Sequence Synthetic polynucleotide
ttaacaacaacgaaggggctgtgactggctgctttctcaaccaatcagcaccgaactcatttgcatgggctgagaacaaatgttcgcgaactctagaaatgaatgacttaagtaagttccttagaatattatttttcctactgaaagttaccacatgcgtcgttgtttatacagtaataggaacaagaaaaaagtcacctaagctcaccctcatcaattgtggagttcctttatatcccatcttctctccaaacacatacgca

SEQ ID NO: 1599 - DNA, 257 bp, Artificial Sequence
  Description of Artificial Sequence Synthetic polynucleotide
ttaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgc

SEQ ID NO: 1600 - DNA, 393 bp, Artificial Sequence
  Description of Artificial Sequence Synthetic polynucleotide
taaggaccagcttctttgggagagaacagacgcaggggcgggagggaaaaagggagaggcagacgtcacttcctcttggcgactctggcagcagattggtcggttgagtggcagaaaggcagacggggactgggcaaggcactgtcggtgacatcacggacagggcgacttctatgtagatgaggcagcgcagaggctgctgcttcgccacttgctgcttcgccacgaagggagttcccgtgccctgggagcgggttcaggaccgctgatcggaagtgagaatcccagctgtgtgtcagggctggaaagggctcgggagtgcgcggggcaagtgaccgtgtgtgtaaagagtgaggcgtatgaggctgtgtcggggcagagcccgaagatctc

SEQ ID NO: 1601 - DNA, 698 bp, Artificial Sequence
  Description of Artificial Sequence Synthetic polynucleotide
atacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacc

SEQ ID NO: 1602 - DNA, 105 bp, Artificial Sequence
  Description of Artificial Sequence Synthetic polynucleotide
  modified_base (1)..(1): a, c, t, g, unknown or other
nactgggagtggccattcgacgacagtgtggtgtaaaggaattcattagccatggatgtattcatgaaaggactttcaaaggccaaggagggagttgtggctgct

SEQ ID NO: 1603 - DNA, 105 bp, Artificial Sequence
  Description of Artificial Sequence Synthetic polynucleotide
  modified_base (105)..(105): a, c, t, g, unknown or other
agcagccacaactccgggaacccctttgaaagtcctttcccgaatacatccacggctaaacttctcctttacaccacactgtcgtcgattaggccactcccagtn

(19) United States
(12) Patent Application Publication
(10) Pub. No.: US 2025/0011775 A1
(43) Pub. Date: Jan. 9, 2025

(54) Engineered Guide RNAs and Polynucleotides

(71) Applicants: Adrian BRIGGS, Seattle, WA (US); Brian BOOTH, Seattle, WA (US); Richard SULLIVAN, Seattle, WA (US); Yazmin Ines ROVIRA GONZALEZ, Seattle, WA (US); Shape Therapeutics Inc., Seattle, WA (US)

(72) Inventors: Adrian BRIGGS, Seattle, WA (US); Brian BOOTH, West Roxbury, MA (US); Richard SULLIVAN, Seattle, WA (US); Yazmin Ines ROVIRA GONZALEZ, Seattle, WA (US); Yiannis SAVVA, Seattle, WA (US)

(21) Appl. No.: 18/562,391

(22) PCT Filed: May 23, 2022
(86) PCT No.: PCT/US2022/030503

§ 371 Date: Nov. 20, 2023

Related U.S. Application Data
(60) Provisional application No. 63/303,662, filed on Jan. 27, 2022.
(60) Provisional application No. 63/277,665, filed on Nov. 10, 2021.
(60) Provisional application No. 63/216,175, filed on Jun. 29, 2021.
(60) Provisional application No. 63/192,818, filed on May 25, 2021.

(51) Int. Cl. C12N 15/113; C12N 9/78; C12N 15/11; C12N 15/86

(52) U.S. Cl. CPC C12N 15/113; C12N 9/78; C12N 15/111; C12N 15/86; C12N 2310/11; C12N 2750/14143; C12Y 305/04

(57) ABSTRACT

Disclosed herein are engineered guide RNAs and compositions comprising the same for treatment of diseases or conditions in a subject. Also disclosed herein are methods of treating diseases or conditions in a subject by administering engineered guide RNAs or pharmaceutical compositions described herein.

### CROSS REFERENCE

This application claims priority under 35 U.S.C. § 119 from Provisional Application Ser. No. 63/192,818, filed May 25, 2021, Provisional Application Ser. No. 63/216,175, filed Jun. 29, 2021, Provisional Application Ser. No. 63/277,665, filed Nov. 10, 2021, and Provisional Application Ser. No. 63/303,662, filed Jan. 27, 2022, the disclosures of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on May 19, 2022, is named 199235-733601_SL.txt and is 508,120 bytes in size.

### BACKGROUND

Payloads that mediate RNA editing can be viable therapies for genetic diseases. However, highly efficacious payloads that can maximize on-target RNA editing while minimizing off-target RNA editing are needed. Moreover, payloads that are capable of facilitating RNA editing for protein knockdown are also needed.

### SUMMARY

Disclosed herein are compositions comprising an engineered guide RNA or an engineered polynucleotide encoding the engineered guide RNA. In some embodiments, the engineered guide RNA, upon hybridization to a sequence of a DUX4 target RNA, can form a guide-target RNA scaffold with the sequence of the DUX4 target RNA; formation of the guide-target RNA scaffold substantially forms one or more structural features selected from the group consisting of: a bulge, an internal loop, a hairpin, and a mismatch formed by a base in the engineered guide RNA to a G, a C, or a U in the DUX4 target RNA; and the structural feature may not be present within the engineered guide RNA prior to the hybridization of the engineered guide RNA to the DUX4 target RNA; and upon hybridization of the engineered guide RNA to the sequence of the DUX4 target RNA, the engineered guide RNA can facilitate RNA editing of one or more target adenosines in the sequence of the DUX4 target RNA by an RNA editing entity. In some embodiments, the sequence of the DUX4 target RNA can comprise a translation initiation site, a polyA signal sequence, a splice site, or any combination thereof. In some embodiments, the sequence of the DUX4 target RNA can comprise the polyA signal sequence. In some embodiments, the one or more features can further comprise a mismatch formed by a base in the engineered guide RNA to an A in the DUX4 target RNA. In some embodiments, the DUX4 can be DUX4-FL. In some embodiments, the sequence of the DUX4 target RNA can comprise the polyA signal sequence. In some embodiments, the polyA signal sequence can be in DUX4-FL. In some embodiments, polyA signal sequence can comprise ATTAAA. In some embodiments, any A of the ATTAAA polyA signal sequence can be the target adenosine. In some embodiments, position 0 of ATTAAA can be the target adenosine, wherein position 0 is the first A of ATTAAA at the 5′ end. In some embodiments, the one or more structural features can comprise: a first 6/6 symmetric internal loop at a position selected from the group consisting of: −3, −4, −5, −6, −7, −8, −9, −10, and −11, relative to position 0 of ATTAAA. In some embodiments, the first 6/6 symmetric internal loop can be at position −5 relative to position 0. In some embodiments, the one or more structural features can further comprise a second 6/6 symmetric internal loop at position 33 relative to position 0. In some embodiments, the engineered guide RNA can comprise at least about: 80%, 85%, 90%, 92%, 95%, 97%, or 99% sequence identity to SEQ ID NO: 1054.In some embodiments, the engineered guide RNA can comprise SEQ ID NO: 1054. In some embodiments, the first 6/6 symmetric internal loop is at position −6 relative to position 0. In some embodiments, the one or more structural features can further comprise at least one structural feature selected from the group consisting of: an A/C mismatch at position 3 relative to position 0, a second 6/6 symmetric internal loop at position 42 relative to position 0, and a combination thereof. In some embodiments, the engineered guide RNA can comprise at least about: 80%, 85%, 90%, 92%, 95%, 97%, or 99% sequence identity to SEQ ID NO: 977. In some embodiments, the engineered guide RNA can comprise SEQ ID NO: 977.In some embodiments, the one or more structural features can further comprise at least one structural feature selected from the group consisting of: an A/C mismatch at position 0, a second 6/6 symmetric internal loop at position 33 relative to position 0, and a combination thereof. In some embodiments, the engineered guide RNA can comprise at least about: 80%, 85%, 90%, 92%, 95%, 97%, or 99% sequence identity to SEQ ID NO: 934. In some embodiments, the engineered guide RNA can comprise SEQ ID NO: 934. In some embodiments, the one or more structural features can further comprise at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof. In some embodiments, the engineered guide RNA can comprise at least about: 80%, 85%, 90%, 92%, 95%, 97%, or 99% sequence identity to SEQ ID NO: 1575. In some embodiments, the engineered guide RNA can comprise SEQ ID NO: 1575.In some embodiments, the one or more structural features can further comprise at least one structural feature selected from the group consisting of: an A/C mismatch at position 3 relative to position 0, a second 6/6 symmetric internal loop at position 33 relative to position 0, a 5/5 internal loop at position 47 relative to position 0, a 5/5 internal loop at position 60 relative to position 0, a 5/5 internal loop at position 73 relative to position 0, and any combination thereof. In some embodiments, the engineered guide RNA can comprise at least about: 80%, 85%, 90%, 92%, 95%, 97%, or 99% sequence identity to SEQ ID NO: 1573. In some embodiments, the engineered guide RNA can comprise SEQ ID NO: 1573. In some embodiments, the one or more structural features can further comprise at least one structural feature selected from the group consisting of: an A/C mismatch at position 3 relative to position 0, a second 6/6 symmetric internal loop at position 33 relative to position 0, a 5/5 internal loop at position 45 relative to position 0, a 5/5 internal loop at position 56 relative to position 0, a 5/5 internal loop at position 67 relative to position 0, and any combination thereof. In some embodiments, the engineered guide RNA can comprise at least about: 80%, 85%, 90%, 92%, 95%, 97%, or 99% sequence identity to SEQ ID NO: 1569. In some embodiments, the engineered guide RNA can comprise SEQ ID NO: 1569. In some embodiments, the one or more structural features can further comprise at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof. In some embodiments, the engineered guide RNA can comprise at least about: 80%, 85%, 90%, 92%, 95%, 97%, or 99% sequence identity to SEQ ID NO: 1567. In some embodiments, the engineered guide RNA can comprise SEQ ID NO: 1567. In some embodiments, the one or more structural features can further comprise at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof. In some embodiments, the engineered guide RNA can comprise at least about: 80%, 85%, 90%, 92%, 95%, 97%, or 99% sequence identity to SEQ ID NO: 1588. In some embodiments, the engineered guide RNA can comprise SEQ ID NO: 1588. In some embodiments, the first 6/6 symmetric internal loop is at position −9 relative to position 0. In some embodiments, the one or more structural features can further comprise at least one structural feature selected from the group consisting of: an A/C mismatch at position 0, a second 6/6 symmetric internal loop at position 40 relative to position 0, and a combination thereof. In some embodiments, the engineered guide RNA can comprise at least about: 80%, 85%, 90%, 92%, 95%, 97%, or 99% sequence identity to SEQ ID NO: 593. In some embodiments, the engineered guide RNA can comprise SEQ ID NO: 593. In some embodiments, position 3 of ATTAAA is the target adenosine, wherein position 3 is the second A of ATTAAA from the 5′ end. In some embodiments, the one or more structural features can comprise: a first 6/6 symmetric internal loop at a position selected from the group consisting of: 22, 21, 20, −2, −4, −5, −6, −7, −8, −9, and −10 relative to position 0 of ATTAAA. In some embodiments, the first 6/6 symmetric internal loop is at position 20 relative to position 0. In some embodiments, the one or more structural features can further comprise an A/C mismatch at position 3 relative to position 0. In some embodiments, the engineered guide RNA can comprise at least about: 80%, 85%, 90%, 92%, 95%, 97%, or 99% sequence identity to SEQ ID NO: 8. In some embodiments, the engineered guide RNA can comprise SEQ ID NO: 8. In some embodiments, the first 6/6 symmetric internal loop is at position −5 relative to position 0. In some embodiments, the one or more structural features can further comprise a second 6/6 symmetric internal loop at position 33 relative to position 0. In some embodiments, the engineered guide RNA can comprise at least about: 80%, 85%, 90%, 92%, 95%, 97%, or 99% sequence identity to SEQ ID NO: 1054. In some embodiments, the engineered guide RNA can comprise SEQ ID NO: 1054. In some embodiments, the first 6/6 symmetric internal loop is at position −6 relative to position 0. In some embodiments, the one or more structural features can further comprise at least one structural feature selected from the group consisting of: an A/C mismatch at position 3 relative to position 0, a second 6/6 symmetric internal loop at position 42 relative to position 0, and a combination thereof. In some embodiments, the engineered guide RNA can comprise at least about: 80%, 85%, 90%, 92%, 95%, 97%, or 99% sequence identity to SEQ ID NO: 977. In some embodiments, the engineered guide RNA can comprise SEQ ID NO: 977. In some embodiments, the one or more structural features can further comprise at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof. In some embodiments, the engineered guide RNA can comprise at least about: 80%, 85%, 90%, 92%, 95%, 97%, or 99% sequence identity to SEQ ID NO: 1569. In some embodiments, the engineered guide RNA can comprise SEQ ID NO: 1569. In some embodiments, the one or more structural features can further comprise at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof. In some embodiments, the engineered guide RNA can comprise at least about: 80%, 85%, 90%, 92%, 95%, 97%, or 99% sequence identity to SEQ ID NO: 1567. In some embodiments, the engineered guide RNA can comprise SEQ ID NO: 1567. In some embodiments, the one or more structural features can further comprise at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof. In some embodiments, the engineered guide RNA can comprise at least about: 80%, 85%, 90%, 92%, 95%, 97%, or 99% sequence identity to SEQ ID NO: 1573. In some embodiments, the engineered guide RNA can comprise SEQ ID NO: 1573. In some embodiments, the one or more structural features can further comprise at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof. In some embodiments, the engineered guide RNA can comprise at least about: 80%, 85%, 90%, 92%, 95%, 97%, or 99% sequence identity to SEQ ID NO: 1588. In some embodiments, the engineered guide RNA can comprise SEQ ID NO: 1588. In some embodiments, the one or more structural features can further comprise at least one structural feature selected from the group consisting of: A/C mismatch at position 3, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof. In some embodiments, the engineered guide RNA can comprise at least about: 80%, 85%, 90%, 92%, 95%, 97%, or 99% sequence identity to SEQ ID NO: 1575. In some embodiments, the engineered guide RNA can comprise SEQ ID NO: 1575. In some embodiments, the first 6/6 symmetric internal loop is at position −9 relative to position 0. In some embodiments, the one or more structural features can further comprise at least one structural feature selected from the group consisting of: an A/C mismatch at position 0, a second 6/6 symmetric internal loop at position 40 relative to position 0, and a combination thereof. In some embodiments, the engineered guide RNA can comprise at least about: 80%, 85%, 90%, 92%, 95%, 97%, or 99% sequence identity to SEQ ID NO: 593. In some embodiments, the engineered guide RNA can comprise SEQ ID NO: 593. In some embodiments, the one or more structural features can comprise: a first 2/2 symmetric bulge at a position selected from the group consisting of: −3, −5, and −7 relative to position 0 of ATTAAA. In some embodiments, the first 2/2 symmetric bulge is at position −5 relative to position 0. In some embodiments, the one or more structural features can further comprise at least one structural feature selected from the group consisting of: a 2/2 symmetric bulge at position 26 relative to position 0, a 2/2 symmetric bulge at position 42 relative to position 0, a 2/2 symmetric bulge at position 58 relative to position 0, a 2/2 symmetric bulge at position 74 relative to position 0, and any combination thereof. In some embodiments, the engineered guide RNA can comprise at least about: 80%, 85%, 90%, 92%, 95%, 97%, or 99% sequence identity to SEQ ID NO: 1545. In some embodiments, the engineered guide RNA can comprise SEQ ID NO: 1545.In some embodiments, position 4 of ATTAAA is the target adenosine, wherein position 4 is the third A of ATTAAA from the 5′ end. In some embodiments, the one or more structural features can comprise: a first 6/6 symmetric internal loop at a position selected from the group consisting of: 33, −1, −2, −3, −4, −5, −6, −7, −8, −9, −11, and −12 relative to position 0 of ATTAAA. In some embodiments, the first 6/6 symmetric internal loop is at position −1 relative to position 0.

In some embodiments, the one or more structural features can further comprise at least one structural feature selected from the group consisting of: an A/C mismatch at position 4 relative to position 0, a second 6/6 symmetric internal loop at position 32 relative to position 0, and a combination thereof. In some embodiments, the engineered guide RNA can comprise at least about: 80%, 85%, 90%, 92%, 95%, 97%, or 99% sequence identity to SEQ ID NO: 1463. In some embodiments, the engineered guide RNA can comprise SEQ ID NO: 1463. In some embodiments, the first 6/6 symmetric internal loop is at position −3 relative to position 0. In some embodiments, the one or more structural features can further comprise at least one structural feature selected from the group consisting of: an A/C mismatch at position 4 relative to position 0, a second 6/6 symmetric internal loop at position 36 relative to position 0, and a combination thereof. In some embodiments, the engineered guide RNA can comprise at least about: 80%, 85%, 90%, 92%, 95%, 97%, or 99% sequence identity to SEQ ID NO: 1294. In some embodiments, the engineered guide RNA can comprise SEQ ID NO: 1294. In some embodiments, the first 6/6 symmetric internal loop is at position −5 relative to position 0. In some embodiments, the one or more structural features can further comprise a second 6/6 symmetric internal loop at position 33 relative to position 0. In some embodiments, the engineered guide RNA can comprise at least about: 80%, 85%, 90%, 92%, 95%, 97%, or 99% sequence identity to SEQ ID NO: 1054. In some embodiments, the engineered guide RNA can comprise SEQ ID NO: 1054. In some embodiments, the first 6/6 symmetric internal loop is at position −6 relative to position 0. In some embodiments, the one or more structural features can further comprise at least one structural feature selected from the group consisting of: an A/C mismatch at position 0, a second 6/6 symmetric internal loop at position 33 relative to position 0, and a combination thereof. In some embodiments, the engineered guide RNA can comprise at least about: 80%, 85%, 90%, 92%, 95%, 97%, or 99% sequence identity to SEQ ID NO: 934. In some embodiments, the engineered guide RNA can comprise SEQ ID NO: 934. In some embodiments, the one or more structural features can further comprise at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof. In some embodiments, the engineered guide RNA can comprise at least about: 80%, 85%, 90%, 92%, 95%, 97%, or 99% sequence identity to SEQ ID NO: 1573. In some embodiments, the engineered guide RNA can comprise SEQ ID NO: 1573. In some embodiments, the one or more structural features can further comprise at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof. In some embodiments, the engineered guide RNA can comprise at least about: 80%, 85%, 90%, 92%, 95%, 97%, or 99% sequence identity to SEQ ID NO: 1575. In some embodiments, the engineered guide RNA can comprise SEQ ID NO: 1575. In some embodiments, the one or more structural features can further comprise at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof. In some embodiments, the engineered guide RNA can comprise at least about: 80%, 85%, 90%, 92%, 95%, 97%, or 99% sequence identity to SEQ ID NO: 1567. In some embodiments, the engineered guide RNA can comprise SEQ ID NO: 1567. In some embodiments, the one or more structural features can further comprise at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof. In some embodiments, the engineered guide RNA can comprise at least about: 80%, 85%, 90%, 92%, 95%, 97%, or 99% sequence identity to SEQ ID NO: 1569. In some embodiments, the engineered guide RNA can comprise SEQ ID NO: 1569. In some embodiments, the one or more structural features can further comprise at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof. In some embodiments, the engineered guide RNA can comprise at least about: 80%, 85%, 90%, 92%, 95%, 97%, or 99% sequence identity to SEQ ID NO: 1588. In some embodiments, the engineered guide RNA can comprise SEQ ID NO: 1588. In some embodiments, the first 6/6 symmetric internal loop is at position −9 relative to position 0. In some embodiments, the one or more structural features can further comprise at least one structural feature selected from the group consisting of: an A/C mismatch at position 0, a second 6/6 symmetric internal loop at position 40 relative to position 0, and a combination thereof. In some embodiments, the engineered guide RNA can comprise at least about: 80%, 85%, 90%, 92%, 95%, 97%, or 99% sequence identity to SEQ ID NO: 593. In some embodiments, the engineered guide RNA can comprise SEQ ID NO: 593. In some embodiments, position 5 of ATTAAA is the target adenosine, wherein position 5 is the forth A of ATTAAA from the 5′ end. In some embodiments, the one or more structural features can comprise:a first 6/6 symmetric internal loop at a position selected from the group consisting of: 33, 23, −1, −2, −3, −4, −5, −6, −7, −8, −9, −10, and −12 relative to position 0 of ATTAAA. In some embodiments, the first 6/6 symmetric internal loop is at position −1 relative to position 0. In some embodiments, the one or more structural features can further comprise at least one structural feature selected from the group consisting of: an A/C mismatch at position 4 relative to position 0, a second 6/6 symmetric internal loop at position 32 relative to position 0, and a combination thereof. In some embodiments, the engineered guide RNA can comprise at least about: 80%, 85%, 90%, 92%, 95%, 97%, or 99% sequence identity to SEQ ID NO: 1463. In some embodiments, the engineered guide RNA can comprise SEQ ID NO: 1463. In some embodiments, the first 6/6 symmetric internal loop is at position −5 relative to position 0. In some embodiments, the one or more structural features can further comprise a second 6/6 symmetric internal loop at position 33 relative to position 0. In some embodiments, the engineered guide RNA can comprise at least about: 80%, 85%, 90%, 92%, 95%, 97%, or 99% sequence identity to SEQ ID NO: 1054. In some embodiments, the engineered guide RNA can comprise SEQ ID NO: 1054. In some embodiments, the first 6/6 symmetric internal loop is at position −6 relative to position 0. In some embodiments, the one or more structural features can further comprise at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof. In some embodiments, the engineered guide RNA can comprise at least about: 80%, 85%, 90%, 92%, 95%, 97%, or 99% sequence identity to SEQ ID NO: 1575. In some embodiments, the engineered guide RNA can comprise SEQ ID NO: 1575. In some embodiments, the one or more structural features can further comprise at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof. In some embodiments, the engineered guide RNA can comprise at least about: 80%, 85%, 90%, 92%, 95%, 97%, or 99% sequence identity to SEQ ID NO: 1567. In some embodiments, the engineered guide RNA can comprise SEQ ID NO: 1567. In some embodiments, the one or more structural features can further comprise at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof. In some embodiments, the engineered guide RNA can comprise at least about: 80%, 85%, 90%, 92%, 95%, 97%, or 99% sequence identity to SEQ ID NO: 1573. In some embodiments, the engineered guide RNA can comprise SEQ ID NO: 1573. In some embodiments, the one or more structural features can further comprise at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof. In some embodiments, the engineered guide RNA can comprise at least about: 80%, 85%, 90%, 92%, 95%, 97%, or 99% sequence identity to SEQ ID NO: 1569. In some embodiments, the engineered guide RNA can comprise SEQ ID NO: 1569. In some embodiments, the one or more structural features can further comprise at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof. In some embodiments, the engineered guide RNA can comprise at least about: 80%, 85%, 90%, 92%, 95%, 97%, or 99% sequence identity to SEQ ID NO: 1588. In some embodiments, the engineered guide RNA can comprise SEQ ID NO: 1588. In some embodiments, the method can further comprise editing at any A of ATTAAA. In some embodiments, the one or more structural features comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 8. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 593. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 934. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 977. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1054. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1294. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1463. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1545. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1567. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1569. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1573. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1575. In some embodiments, the one or more structural features can comprise a 1 nucleotide mismatch formed 3 nucleotides downstream (3′) from the target A, and a 6 nucleotide internal symmetric loop formed 20 nucleotides downstream (3′) from the target A. In some embodiments, the engineered guide RNA can have at least 80%, 85%, 90%, 92%, 95%, 97%, or 99% sequence identity to a guide RNA comprising SEQ ID NO: 1588. In some embodiments, the one or more structural features can comprise: a first 6/6 symmetric internal loop, and at least one additional structural feature selected from the group consisting of: a second 6/6 symmetric internal loop, a 5/5 symmetric internal loop, a 4/4 symmetric bulge, a 3/3 symmetric bulge, and a 2/2 symmetric bulge. In some embodiments, the guide-target RNA scaffold can further comprise an A/C mismatch, wherein the cytosine of the A/C mismatch is present in the engineered guide RNA opposite the one or more target adenosines; and wherein the one or more structural features comprise: the first 6/6 symmetric internal loop positioned from position −4 to −8, relative to the A/C mismatch; the second 6/6 symmetric internal loop positioned from position+31 to +35, relative to the A/C mismatch.

In some embodiments, the guide-target RNA scaffold can further comprise an A/C mismatch, wherein the cytosine of the A/C mismatch is present in the engineered guide RNA opposite the one or more target adenosines; and wherein the one or more structural features comprise: the first 6/6 symmetric internal loop at position −6, relative to the A/C mismatch; the second 6/6 symmetric internal loop at position+33, relative to the A/C mismatch. In some embodiments, the first 6/6 symmetric internal loop can comprise the sequence GGAACU on the engineered guide RNA side, and the sequence UUCAGA on the target RNA side.In some embodiments, the second 6/6 symmetric internal loop can comprise the sequence CUGACC on the engineered guide RNA side, and the sequence AGAUUU on the target RNA side. In some embodiments, the one or more structural features can comprise a first 6/6 symmetric internal loop and a second 6/6 symmetric internal loop and wherein each A in the target RNA is base paired to a U in the engineered guide RNA. In some embodiments, the one or more structural features can comprise the bulge. In some embodiments, the bulge can be a symmetric bulge. In some embodiments, the one or more structural features can comprise the bulge. In some embodiments, the bulge can be an asymmetric bulge. In some embodiments, the one or more structural features can comprise the internal loop, wherein the internal loop is a symmetric internal loop. In some embodiments, the one or more structural features can comprise the internal loop. In some embodiments, the internal loop can be an asymmetric internal loop. In some embodiments, the one or more structural features can comprise the mismatch formed by a base in the engineered guide RNA to a G, a C, or a U in the DUX4 target RNA. In some embodiments, the RNA editing entity can comprise ADAR1, ADAR2, ADAR3, or any combination thereof. In some embodiments, the RNA editing of one or more target adenosines can comprise hyper-editing. In some embodiments, the hyper-editing can comprise editing of more than one A in the polyA signal sequence of the DUX4 target RNA. In some embodiments, the internal loop of the engineered guide RNA can comprise any nucleotide in any positional order. In some embodiments, the nucleotide in any positional order is not complementary to their positional counterpart in the DUX 4 target RNA. In some embodiments, the engineered guide RNA or the engineered polynucleotide encoding the engineered guide RNA can be circular. In some embodiments, the engineered guide RNA or the engineered polynucleotide encoding the engineered guide RNA can comprise a U7 hairpin sequence, a SmOPT sequence, or a combination thereof and optionally wherein the U7 hairpin sequence can comprise SEQ ID NO 1591 or 1593 and wherein the SmOPT sequence can comprise SEQ ID NO: 1595. In some embodiments, the DUX4 target RNA can comprise a pre-mRNA transcript of DUX4. In some embodiments, at least 40%, at least 50%, at least 60%, at least 70%, at least 80%, at least 85%, at least 90%, at least 95%, or at least 99% of the pre-mRNA transcripts of DUX4 can have at least one edit in the polyA signal sequence. In some embodiments, at least 80% of the pre-mRNA transcripts of DUX4 can have at least one edit in the polyA signal sequence. In some embodiments, the editing of one or more adenosines can facilitate a mRNA knockdown. In some embodiments, the mRNA knockdown can comprise a knockdown of DUX4 mRNA. In some embodiments, the mRNA knockdown can comprise a reduction of at least 40%, at least 50%, at least 60%, at least 70%, at least 80%, at least 85%, at least 90%, at least 95%, or at least 99% of a mRNA level after RNA editing as compared to a mRNA level before RNA editing. In some embodiments, the mRNA knockdown can be at least 50% of the mRNA level as compared to the mRNA level before RNA editing. In some embodiments, the mRNA knockdown can be at least 70% of the mRNA level as compared to the mRNA level before RNA editing. In some embodiments, the editing of one or more adenosines can facilitate a protein knockdown. In some embodiments, the protein knockdown can comprise a knockdown of DUX4. In some embodiments, the protein knockdown can comprise a knockdown of a protein downstream of DUX4. In some embodiments, the protein downstream of DUX4 can comprise SLC34A2, LEUTX, ZSCAN4, PRAMEF12, TRIM43, DEFB103, or MBD3L2, or any combination thereof. In some embodiments, the protein knockdown can comprise a reduction of at least 40%, at least 50%, at least 60%, at least 70%, at least 80%, at least 85%, at least 90%, at least 95%, or at least 99% of the protein level after RNA editing as compared to the protein level before RNA editing. In some embodiments, the protein knockdown can comprise a reduction of at least 40%, at least 50%, at least 60%, at least 70%, at least 80%, at least 85%, at least 90%, at least 95%, or at least 99% of the protein level in an ADAR expressing cell as compared to a cell comprising an nonfunctional ADAR gene. In some embodiments, the protein knockdown can comprise ADAR-dependent protein knockdown. In some embodiments, the ADAR-dependent protein knockdown can comprise a reduction of at least 50%, at least 60%, at least 70%, at least 80%, at least 85%, at least 90%, at least 95%, or at least 99% of the protein level as compared to the protein level before RNA editing. In some embodiments, the engineered guide RNA is an in vitro transcribed (IVT) engineered guide RNA. In some embodiments, the composition can comprise the engineered polynucleotide. In some embodiments, the engineered polynucleotide can be comprised in or on a vector. In some embodiments, the vector can be a viral vector. In some embodiments, the engineered polynucleotide can be encapsidated in the viral vector. In some embodiments, the viral vector can be an adeno-associated viral (AAV) vector or a derivative thereof. In some embodiments, the vector can be a non-viral vector. In some embodiments, the non-viral vector can be a lipid nanoparticle (LNP), a liposome, or a polymer nanoparticle. In some embodiments, the engineered polynucleotide can be a DNA polynucleotide encoding the engineered guide RNA. In some embodiments, the engineered guide RNA can comprise at least 80%, 85%, 90%, 92%, 95%, 97%, or 99% sequence identity to any one of SEQ ID NO: 2-SEQ ID NO: 1589. In some embodiments, the engineered guide RNA can comprise a sequence of any one of SEQ ID NO: 2-SEQ ID NO: 1589.

Also described herein are pharmaceutical compositions comprising: a) any of the compositions described above; and b) a pharmaceutically acceptable: excipient, carrier, or diluent.

Also described herein are methods of treating a disease or a condition in a subject in need thereof, the method comprising administering to the subject a therapeutically effective amount of any of the compositions described above or the pharmaceutical composition described above.

In some embodiments, the disease or condition can comprise facioscapulohumeral muscular dystrophy (FSHD). In some embodiments, FSHD can comprise Type I FSHD. In some embodiments, FSHD can comprise Type II FSHD. In some embodiments, the administering can comprise parenteral administration, intravenous administration, subcutaneous administration, intrathecal administration, intraperitoneal administration, intramuscular administration, intravascular administration, infusion administration, topical administration, oral administration, inhalation administration, intraduodenal administration, rectal administration, or a combination thereof. In some embodiments, the administration can be oral administration. In some embodiments, the administering can comprise systemic administration.

Also described herein are methods of editing a DUX4 RNA. In some embodiments, the method can comprise contacting the DUX4 RNA with any one of the compositions described above and an RNA editing entity, thereby editing the DUX4 RNA. In some embodiments, the editing can comprise editing at any A position of a polyA tail of the DUX4 RNA. In some embodiments, the DUX4 RNA can comprise a pre-mRNA transcript of DUX4. In some embodiments, at least 40%, at least 50%, at least 60%, at least 70%, at least 80%, at least 85%, at least 90%, at least 95%, or at least 99% of the pre-mRNA transcripts of DUX4 have at least one edit in the polyA signal sequence. In some embodiments, the editing of DUX4 RNA can facilitate a protein knockdown. In some embodiments, the protein knockdown can comprise a knockdown of DUX4.

Also described herein are the compositions described above and the pharmaceutical compositions described above for use as a medicament. In some embodiments, a composition described above or a pharmaceutical composition described above can be for use in the treatment of facioscapulohumeral muscular dystrophy (FSHD). In some embodiments, FSHD can comprise Type I FSHD. In some embodiments, FSHD can comprise Type II FSHD.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Novel features of the present disclosure are set forth with particularity in the appended claims. A better understanding of the features and advantages of the present disclosure will be obtained by reference to the following detailed description that sets forth illustrative embodiments, in which exemplary principles of the present disclosure are utilized, and the accompanying drawings of which:

FIG. 1 shows a schematic of the double homeobox 4 (DUX4) target, highlighting sites that can be targeted by engineered guide RNAs of the present disclosure.

FIG. 2 shows a schematic of the DMPK target, highlighting sites that can be targeted by engineered guide RNAs of the present disclosure.

FIG. 3 shows a schematic of the PMP22 target, highlighting sites that can be targeted by engineered guide RNAs of the present disclosure.

FIG. 4 shows a schematic of the SOD1 target, highlighting sites that can be targeted by engineered guide RNAs of the present disclosure.

FIG. 5 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a ⅔ asymmetric bulge (2 nucleotides on the target RNA side and 3 nucleotides on the guide RNA side). This figure discloses SEQ ID NOs: 1602 and 1603, respectively in order of appearance.

FIG. 6 is a plot showing, on the x-axis, the sequence similarity of the DUX4-targeting engineered guide RNA sequences of the present disclosure to a canonical guide RNA design and, on the y-axis, the edited fraction by an ADAR2 enzyme. These data highlight the diverse sequence space represented by the DUX4-targeting engineered guide RNA sequences of the present disclosure, which have a range of different structural features that drive sequence diversity and which exhibit high on-target editing efficiency.

FIG. 7 shows a schematic of the luciferase and GFP reporter constructs designed to determine expression changes of the reporters fused to mutated DUX4-FL polyA site adenosines.

FIG. 8A shows the viability and transfection efficiencies of LHCN cells after transfection with the luciferase reporters.

FIG. 8B shows the mCherry median fluorescent intensity (MFI) of luciferase reporter transfected LHCN cells.

FIG. 8C shows the luciferase signal normalized to mCherry MFI of the luciferase constructs carrying the mutated or wild type DUX4-FL polyA site adenosines.

FIG. 9A shows the viability and transfection efficiencies of LHCN cells after transfection with the GFP reporters.

FIG. 9B shows the mCherry median fluorescent intensity (MFI) of GFP reporter transfected LHCN cells.

FIG. 9C shows the GFP MFI signal normalized to mCherry MFI of the GFP constructs carrying the mutated or wild type DUX4-FL polyA site adenosines.

FIG. 10 shows editing of an integrated DUX4-luciferase reporter in HEK cells with different guide RNAs.

FIG. 11 shows editing of an integrated DUX4-luciferase reporter in ADAR ½ (1 and 2) knockout HEK cells with different guide RNAs.

### DETAILED DESCRIPTION

### RNA Editing

RNA editing can refer to a process by which RNA can be enzymatically modified post synthesis at specific nucleosides. RNA editing can comprise any one of an insertion, deletion, or substitution of a nucleotide(s). Examples of RNA editing include chemical modifications, such as pseudouridylation (the isomerization of uridine residues) and deamination (removal of an amine group from cytidine to give rise to uridine, or C-to-U editing or from adenosine to inosine, or A-to-I editing). RNA editing can be used to introduce mutations, correct missense mutations, or edit coding or non-coding regions of RNA to inhibit RNA translation and effect protein knockdown.

Described herein are engineered guide RNAs that facilitate RNA editing by an RNA editing entity (e.g., an adenosine Deaminase Acting on RNA (ADAR)) or biologically active fragments thereof. In some instances, ADARs can be enzymes that catalyze the chemical conversion of adenosines to inosines in RNA. Because the properties of inosine mimic those of guanosine (inosine will form two hydrogen bonds with cytosine, for example), inosine can be recognized as guanosine by the translational cellular machinery. “Adenosine-to-inosine (A-to-I) RNA editing”, therefore, effectively changes the primary sequence of RNA targets. In general, ADAR enzymes share a common domain architecture comprising a variable number of amino-terminal dsRNA binding domains (dsRBDs) and a single carboxy-terminal catalytic deaminase domain. Human ADARs possess two or three dsRBDs. Evidence suggests that ADARs can form homodimer as well as heterodimer with other ADARs when bound to double-stranded RNA, however it can be currently inconclusive if dimerization is needed for editing to occur. The engineered guide RNAs disclosed herein can facilitate RNA editing by any of or any combination of the three human ADAR genes that have been identified (ADARs 1-3). ADARs have a typical modular domain organization that includes at least two copies of a dsRNA binding domain (dsRBD; ADAR1 with three dsRBDs; ADAR2 and ADAR3 each with two dsRBDs) in their N-terminal region followed by a C-terminal deaminase domain. The engineered guide RNAs of the present disclosure facilitate RNA editing by endogenous ADAR enzymes. In some embodiments, exogenous ADAR can be delivered alongside the engineered guide RNAs disclosed herein.

The present disclosure, in some embodiments, provides engineered guide RNAs that facilitate edits at particular regions in a target RNA (e.g., mRNA or pre-mRNA). For example, the engineered guide RNAs disclosed herein can target a coding sequence of an RNA. A target region in a coding sequence of an RNA can be a translation initiation site (TIS). The engineered guide RNAs disclosed herein can target a non-coding sequence of an RNA, for example, a polyadenylation (polyA) signal sequence in the 3′UTR. The engineered guide RNAs disclosed herein can target a splice site. In some cases, a splice site can be present pre-mRNA (prior to processing to remove introns).

The present disclosure, in some embodiments, provides engineered guide RNAs that facilitate edits at multiple adenosines. Hydrolytic deamination of multiple adenosines in an RNA can be referred to as hyper-editing. In some cases, hyper-editing can occur in cis (e.g. in an Alu element) or in trans (e.g. in a target RNA by an engineered guide RNA). In some cases, hyper-editing can comprise editing in the polyA signal sequence of the DUX4-FL target RNA. In some cases, hyper-editing can introduce edits in at least 2 or more nucleotides of a subject target RNA. In some cases, hyper-editing can introduce at least or at most about 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, or at least or at most about 100 edits in a region of a target RNA. In an embodiment, hyper-editing can occur in an untranslated region, translated region, 3′UTR, 5′UTR, or any combinations thereof.

TIS. In some embodiments, the engineered guide RNAs of the present disclosure target the adenosine at a translation initiation site (TIS). The engineered guide RNAs facilitate ADAR-mediated RNA editing of the TIS (AUG) to GUG. This results in inhibition of RNA translation and, thereby, protein knockdown.

Splice site. In some embodiments, the engineered guide RNAs of the present disclosure target an adenosine at a splice site. The engineered guide RNAs facilitate ADAR-mediated RNA editing of an A at a splice site. This can result in mistranslation and/or truncation of a protein encoded by the pre-mRNA molecule and, thereby, protein knockdown.

PolyA Signal Sequence. In some embodiments, the engineered guide RNAs of the present disclosure target one or more adenosines in the polyA signal sequence. In some embodiments, an engineered guide RNA facilitates ADAR-mediated RNA editing of the one or more adenosines in the polyA signal sequence, thereby resulting in disruption of RNA processing and degradation of the target mRNA and, thereby, protein knockdown. In some embodiments, a target can have one or more polyA signal sequences. In these instances, one or more engineered guide RNAs, varying in their respective sequences, of the present disclosure can be multiplexed to target adenosines in the one or more polyA signal sequences. In both cases, the engineered guide RNAs of the present disclosure facilitated ADAR-mediated RNA editing of adenosines to inosines (read as guanosines by cellular machinery) in the polyA signal sequence, resulting in protein knockdown.

### Engineered Guide RNAs

Disclosed herein are engineered guide RNAs and engineered polynucleotides encoding the same for site-specific, selective editing of a target RNA via an RNA editing entity or a biologically active fragment thereof. An engineered guide RNA of the present disclosure can comprise latent structures, such that when the engineered guide RNA is hybridized to the target RNA to form a guide-target RNA scaffold, at least a portion of the latent structure manifests as at least a portion of a structural feature as described herein.

An engineered guide RNA as described herein comprises a targeting domain with complementarity to a target RNA described herein. As such, a guide RNA can be engineered to site-specifically/selectively target and hybridize to a particular target RNA, thus facilitating editing of specific nucleotide in the target RNA via an RNA editing entity or a biologically active fragment thereof. The targeting domain can include a nucleotide that is positioned such that, when the guide RNA is hybridized to the target RNA, the nucleotide opposes a base to be edited by the RNA editing entity or biologically active fragment thereof and does not base pair, or does not fully base pair, with the base to be edited. This mismatch can help to localize editing of the RNA editing entity to the desired base of the target RNA. However, in some instances there can be some, and in some cases significant, off target editing in addition to the desired edit.

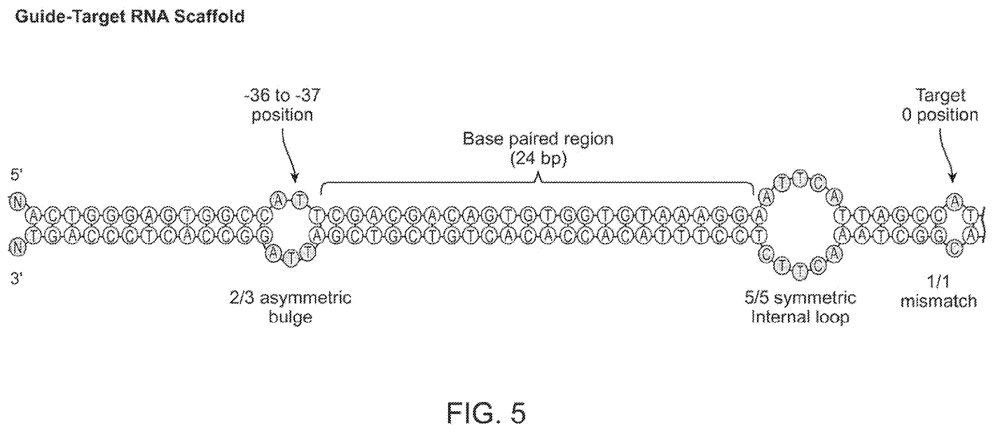

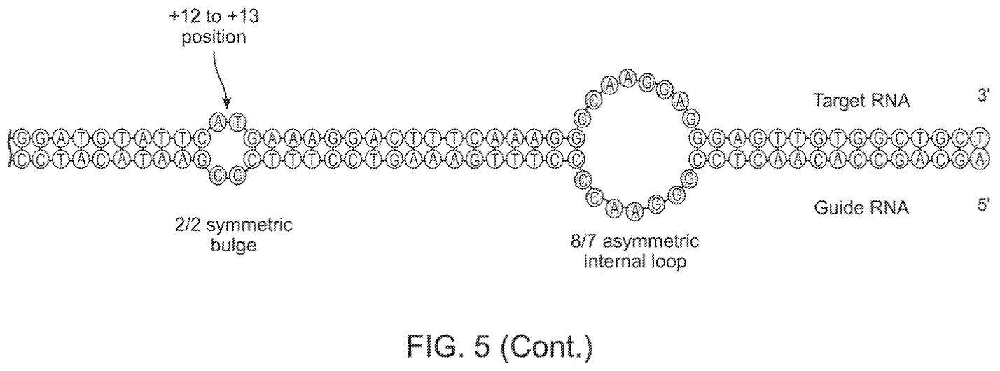

Hybridization of the target RNA and the targeting domain of the guide RNA produces specific secondary structures in the guide-target RNA scaffold that manifest upon hybridization, which are referred to herein as “latent structures.” Latent structures when manifested become structural features described herein, including mismatches, bulges, internal loops, and hairpins. Without wishing to be bound by theory, the presence of structural features described herein that are produced upon hybridization of the guide RNA with the target RNA configure the guide RNA to facilitate a specific, or selective, targeted edit of the target RNA via the RNA editing entity or biologically active fragment thereof. Further, the structural features in combination with the mismatch described above generally facilitate an increased amount of editing of a target adenosine, fewer off target edits, or both, as compared to a construct comprising the mismatch alone or a construct having perfect complementarity to a target RNA. Accordingly, rational design of latent structures in engineered guide RNAs of the present disclosure to produce specific structural features in a guide-target RNA scaffold can be a powerful tool to promote editing of the target RNA with high specificity, selectivity, and robust activity. FIG. 5 illustrates a target RNA scaffold with exemplary structural features.

Provided herein are engineered guides and polynucleotides encoding the same; as well as compositions comprising said engineered guide RNAs or said polynucleotides. As used herein, the term “engineered” in reference to a guide RNA or polynucleotide encoding the same refers to a non-naturally occurring guide RNA or polynucleotide encoding the same. For example, the present disclosure provides for engineered polynucleotides encoding engineered guide RNAs. In some embodiments, the engineered guide comprises RNA. In some embodiments, the engineered guide comprises DNA. In some examples, the engineered guide comprises modified RNA bases or unmodified RNA bases. In some embodiments, the engineered guide comprises modified DNA bases or unmodified DNA bases. In some examples, the engineered guide comprises both DNA and RNA bases.

In some examples, the engineered guides provided herein comprise an engineered guide that can be configured, upon hybridization to a target RNA molecule, to form, at least in part, a guide-target RNA scaffold with at least a portion of the target RNA molecule, wherein the guide-target RNA scaffold comprises at least one structural feature, and wherein the guide-target RNA scaffold recruits an RNA editing entity and facilitates a chemical modification of a base of a nucleotide in the target RNA molecule by the RNA editing entity.

In some examples, a target RNA of an engineered guide RNA of the present disclosure can be a pre-mRNA or mRNA. In some embodiments, the engineered guide RNA of the present disclosure hybridizes to a sequence of the target RNA. In some embodiments, part of the engineered guide RNA (e.g., a targeting domain) hybridizes to the sequence of the target RNA. The part of the engineered guide RNA that hybridizes to the target RNA is of sufficient complementary to the sequence of the target RNA for hybridization to occur.

### A. Targeting Domain

Engineered guide RNAs disclosed herein can be engineered in any way suitable for RNA editing. In some examples, an engineered guide RNA generally comprises at least a targeting sequence that allows it to hybridize to a region of a target RNA molecule. A targeting sequence can also be referred to as a “targeting domain” or a “targeting region”.

In some cases, a targeting domain of an engineered guide allows the engineered guide to target an RNA sequence through base pairing, such as Watson Crick base pairing. In some examples, the targeting sequence can be located at either the N-terminus or C-terminus of the engineered guide. In some cases, the targeting sequence can be located at both termini. The targeting sequence can be of any length. In some cases, the targeting sequence can be at least about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or up to about 200 nucleotides in length. In some cases, the targeting sequence can be no greater than about: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or 200 nucleotides in length. In some examples, an engineered guide comprises a targeting sequence that can be from about 60 to about 500, from about 60 to about 200, from about 75 to about 100, from about 80 to about 200, from about 90 to about 120, or from about 95 to about 115 nucleotides in length. In some examples, an engineered guide RNA comprises a targeting sequence that can be about 100 nucleotides in length.

In some cases, a targeting domain comprises 95%, 96%, 97%, 98%, 99%, or 100% sequence complementarity to a target RNA. In some cases, a targeting sequence comprises less than 100% complementarity to a target RNA sequence. For example, a targeting sequence and a region of a target RNA that can be bound by the targeting sequence can have a single base mismatch.

### B. Engineered Guide RNAs Having a Recruiting Domain

In some examples, a subject engineered guide RNA comprises a recruiting domain that recruits an RNA editing entity (e.g., ADAR), where in some instances, the recruiting domain is formed and present in the absence of binding to the target RNA. A “recruiting domain” can be referred to herein as a “recruiting sequence” or a “recruiting region”. In some examples, a subject engineered guide can be configured to facilitate editing of a base of a nucleotide of a polynucleotide of a region of a subject target RNA, modulation expression of a polypeptide encoded by the subject target RNA, or both. In some cases, an engineered guide can be configured to facilitate an editing of a base of a nucleotide or polynucleotide of a region of an RNA by a subject RNA editing entity. In order to facilitate editing, an engineered guide RNA of the disclosure can recruit an RNA editing entity. Various RNA editing entity recruiting domains can be utilized. In some examples, a recruiting domain comprises: Glutamate ionotropic receptor AMPA type subunit 2 (GluR2), APOBEC, or Alu.

In some examples, more than one recruiting domain can be included in an engineered guide of the disclosure. In examples where a recruiting domain can be present, the recruiting domain can be utilized to position the RNA editing entity to effectively react with a subject target RNA after the targeting sequence, for example an antisense sequence, hybridizes to a target RNA. In some cases, a recruiting domain can allow for transient binding of the RNA editing entity to the engineered guide. In some examples, the recruiting domain allows for permanent binding of the RNA editing entity to the engineered guide. A recruiting domain can be of any length. In some cases, a recruiting domain can be from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, up to about 80 nucleotides in length. In some cases, a recruiting domain can be no more than about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, or 80 nucleotides in length. In some cases, a recruiting domain can be about 45 nucleotides in length. In some cases, at least a portion of a recruiting domain comprises at least 1 to about 75 nucleotides. In some cases, at least a portion of a recruiting domain comprises about 45 nucleotides to about 60 nucleotides.

In an embodiments, a recruiting domain comprises a GluR2 sequence or functional fragment thereof. In some cases, a GluR2 sequence can be recognized by an RNA editing entity, such as an ADAR or biologically active fragment thereof. In some embodiments, a GluR2 sequence can be a non-naturally occurring sequence. In some cases, a GluR2 sequence can be modified, for example for enhanced recruitment. In some embodiments, a GluR2 sequence can comprise a portion of a naturally occurring GluR2 sequence and a synthetic sequence.

In some examples, a recruiting domain comprises a GluR2 sequence, or a sequence having at least about 70%, 80%, 85%, 90%, 95%, 98%, 99%, or 100% identity and/or length to: GUGGAAUAGUAUAACAAUAUGCUAAAUGUUGUUAUAGUAUCCCAC (SEQ ID NO: 1). In some cases, a recruiting domain can comprise at least about 80% sequence homology to at least about 10, 15, 20, 25, or 30 nucleotides of SEQ ID NO: 1. In some examples, a recruiting domain can comprise at least about 90%, 95%, 96%, 97%, 98%, or 99% sequence homology and/or length to SEQ ID NO: 1.

Additional, RNA editing entity recruiting domains are also contemplated. In an embodiment, a recruiting domain comprises an apolipoprotein B mRNA editing enzyme, catalytic polypeptide-like (APOBEC) domain. In some cases, an APOBEC domain can comprise a non-naturally occurring sequence or naturally occurring sequence. In some embodiments, an APOBEC-domain-encoding sequence can comprise a modified portion. In some cases, an APOBEC-domain-encoding sequence can comprise a portion of a naturally occurring APOBEC-domain-encoding-sequence. In another embodiment, a recruiting domain can be from an Alu domain.

Any number of recruiting domains can be found in an engineered guide of the present disclosure. In some examples, at least about 1, 2, 3, 4, 5, 6, 7, 8, 9, or up to about 10 recruiting domains can be included in an engineered guide. Recruiting domains can be located at any position of subject guides. In some cases, a recruiting domain can be on an N-terminus, middle, or C-terminus of a polynucleotide. A recruiting domain can be upstream or downstream of a targeting sequence. In some cases, a recruiting domain flanks a targeting sequence of a subject guide. A recruiting sequence can comprise all ribonucleotides or deoxyribonucleotides, although a recruiting domain comprising both ribo- and deoxyribonucleotides can in some cases not be excluded.

C. Engineered Guide RNAs with Latent Structure

In some examples, an engineered guide disclosed herein useful for facilitating editing of a target RNA by an RNA editing entity can be an engineered latent guide RNA. An “engineered latent guide RNA” refers to an engineered guide RNA that comprises latent structure. “Latent structure” refers to a structural feature that substantially forms upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

A double stranded RNA (dsRNA) substrate is formed upon hybridization of an engineered guide RNA of the present disclosure to a target RNA. The resulting dsRNA substrate is also referred to herein as a “guide-target RNA scaffold.”

FIG. 5 shows a legend of various exemplary structural features present in guide-target RNA scaffolds formed upon hybridization of a latent guide RNA of the present disclosure to a target RNA. Example structural features shown include an 8/7 asymmetric loop (8 nucleotides on the target RNA side and 7 nucleotides on the guide RNA side), a 2/2 symmetric bulge (2 nucleotides on the target RNA side and 2 nucleotides on the guide RNA side), a 1/1 mismatch (1 nucleotide on the target RNA side and 1 nucleotide on the guide RNA side), a 5/5 symmetric internal loop (5 nucleotides on the target RNA side and 5 nucleotides on the guide RNA side), a 24 bp region (24 nucleotides on the target RNA side base paired to 24 nucleotides on the guide RNA side), and a ⅔ asymmetric bulge (2 nucleotides on the target RNA side and 3 nucleotides on the guide RNA side). Unless otherwise noted, the number of participating nucleotides in a given structural feature is indicated as the nucleotides on the target RNA side over nucleotides on the guide RNA side. Also shown in this legend is a key to the positional annotation of each figure. For example, the target nucleotide to be edited is designated as the 0 position. Downstream (3′) of the target nucleotide to be edited, each nucleotide is counted in increments of +1. Upstream (5′) of the target nucleotide to be edited, each nucleotide is counted in increments of −1. Thus, the example 2/2 symmetric bulge in this legend is at the +12 to +13 position in the guide-target RNA scaffold. Similarly, the ⅔ asymmetric bulge in this legend is at the −36 to −37 position in the guide-target RNA scaffold. As used herein, positional annotation is provided with respect to the target nucleotide to be edited and on the target RNA side of the guide-target RNA scaffold. As used herein, if a single position is annotated, the structural feature extends from that position away from position 0 (target nucleotide to be edited). For example, if a latent guide RNA is annotated herein as forming a ⅔ asymmetric bulge at position −36, then the ⅔ asymmetric bulge forms from −36 position to the −37 position with respect to the target nucleotide to be edited (position 0) on the target RNA side of the guide-target RNA scaffold. As another example, if a latent guide RNA is annotated herein as forming a 2/2 symmetric bulge at position+12, then the 2/2 symmetric bulge forms from the +12 to the +13 position with respect to the target nucleotide to be edited (position 0) on the target RNA side of the guide-target RNA scaffold.

In some examples, the engineered guides disclosed herein lack a recruiting region and recruitment of the RNA editing entity can be effectuated by structural features of the guide-target RNA scaffold formed by hybridization of the engineered guide RNA and the target RNA. In some examples, the engineered guide, when present in an aqueous solution and not bound to the target RNA molecule, does not comprise structural features that recruit the RNA editing entity (e.g., ADAR). The engineered guide RNA, upon hybridization to a target RNA, form with the target RNA molecule, one or more structural features that recruits an RNA editing entity (e.g., ADAR).

In cases where a recruiting sequence can be absent, an engineered guide RNA can be still capable of associating with a subject RNA editing entity (e.g., ADAR) to facilitate editing of a target RNA and/or modulate expression of a polypeptide encoded by a subject target RNA. This can be achieved through structural features formed in the guide-target RNA scaffold formed upon hybridization of the engineered guide RNA and the target RNA. Structural features can comprise any one of a: mismatch, symmetrical bulge, asymmetrical bulge, symmetrical internal loop, asymmetrical internal loop, hairpins, wobble base pairs, or any combination thereof.

Described herein are structural features which can be present in a guide-target RNA scaffold of the present disclosure. Examples of features include a mismatch, a bulge (symmetrical bulge or asymmetrical bulge), an internal loop (symmetrical internal loop or asymmetrical internal loop), or a hairpin (a recruiting hairpin or a non-recruiting hairpin). Engineered guide RNAs of the present disclosure can have from 1 to 50 features. Engineered guide RNAs of the present disclosure can have from 1 to 5, from 5 to 10, from 10 to 15, from 15 to 20, from 20 to 25, from 25 to 30, from 30 to 35, from 35 to 40, from 40 to 45, from 45 to 50, from 5 to 20, from 1 to 3, from 4 to 5, from 2 to 10, from 20 to 40, from 10 to 40, from 20 to 50, from 30 to 50, from 4 to 7, or from 8 to 10 features. In some embodiments, structural features (e.g., mismatches, bulges, internal loops) can be formed from latent structure in an engineered latent guide RNA upon hybridization of the engineered latent guide RNA to a target RNA and, thus, formation of a guide-target RNA scaffold. In some embodiments, structural features are not formed from latent structures and are, instead, pre-formed structures (e.g., a GluR2 recruitment hairpin or a hairpin from U7 snRNA).

A guide-target RNA scaffold is formed upon hybridization of an engineered guide RNA of the present disclosure to a target RNA. As disclosed herein, a mismatch refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a bulge or an internal loop, depending on the size of the structural feature. In some embodiments, a mismatch in a guide RNA is to a G, a C, or a U in the DUX4 target RNA. For example, a G in the DUX4 target RNA can mismatch with a G, an A or a U in the guide RNA. In another example, a C in the DUX4 target RNA can mismatch with a C, an A, or a U in the guide RNA. In another example, a U in the DUX4 target RNA can mismatch with a U, a G, or a C in the guide RNA. In some embodiments, a mismatch in a guide RNA is to an A in the DUX4 target RNA. For example, an A in the DUX4 target RNA can mismatch with an A, a G, or a C in the guide RNA. In some embodiments, a mismatch is an A/C mismatch. An A/C mismatch can comprise a C in an engineered guide RNA of the present disclosure opposite an A in a target RNA. An A/C mismatch can comprise an A in an engineered guide RNA of the present disclosure opposite a C in a target RNA. A G/G mismatch can comprise a G in an engineered guide RNA of the present disclosure opposite a G in a target RNA. In some embodiments, a guide RNA of the present disclosure may not have an A/C mismatch and each A of the target RNA is base paired to a U in the engineered guide RNA.

In some embodiments, a mismatch positioned 5′ of the edit site can facilitate base-flipping of the target A to be edited. A mismatch can also help confer sequence specificity. Thus, a mismatch can be a structural feature formed from latent structure provided by an engineered latent guide RNA.

In another aspect, a structural feature comprises a wobble base. A wobble base pair refers to two bases that weakly base pair. For example, a wobble base pair of the present disclosure can refer to a G paired with a U. Thus, a wobble base pair can be a structural feature formed from latent structure provided by an engineered latent guide RNA.

In some cases, a structural feature can be a hairpin. As disclosed herein, a hairpin includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion. A hairpin can have from 10 to 500 nucleotides in length of the entire duplex structure. The loop portion of a hairpin can be from 3 to 15 nucleotides long. A hairpin can be present in any of the engineered guide RNAs disclosed herein. The engineered guide RNAs disclosed herein can have from 1 to 10 hairpins. In some embodiments, the engineered guide RNAs disclosed herein have 1 hairpin. In some embodiments, the engineered guide RNAs disclosed herein have 2 hairpins. As disclosed herein, a hairpin can include a recruitment hairpin or a non-recruitment hairpin. A hairpin can be located anywhere within the engineered guide RNAs of the present disclosure. In some embodiments, one or more hairpins is proximal to or present at the 3′ end of an engineered guide RNA of the present disclosure, proximal to or at the 5′ end of an engineered guide RNA of the present disclosure, proximal to or within the targeting domain of the engineered guide RNAs of the present disclosure, or any combination thereof.

In some aspects, a structural feature comprises a non-recruitment hairpin. A non-recruitment hairpin, as disclosed herein, does not have a primary function of recruiting an RNA editing entity. A non-recruitment hairpin, in some instances, does not recruit an RNA editing entity. In some instances, a non-recruitment hairpin has a dissociation constant for binding to an RNA editing entity under physiological conditions that is insufficient for binding. For example, a non-recruitment hairpin has a dissociation constant for binding an RNA editing entity at 25° C. that is greater than about 1 mM, 10 mM, 100 mM, or 1 M, as determined in an in vitro assay. A non-recruitment hairpin can exhibit functionality that improves localization of the engineered guide RNA to the target RNA. In some embodiments, the non-recruitment hairpin improves nuclear retention. In some embodiments, the non-recruitment hairpin comprises a hairpin from U7 snRNA. Thus, a non-recruitment hairpin such as a hairpin from U7 snRNA is a pre-formed structural feature that can be present in constructs comprising engineered guide RNA constructs, not a structural feature formed by latent structure provided in an engineered latent guide RNA.

A hairpin of the present disclosure can be of any length. In an aspect, a hairpin can be from about 10-500 or more nucleotides. In some cases, a hairpin can comprise about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more nucleotides. In other cases, a hairpin can also comprise 10 to 20, 10 to 30, 10 to 40, 10 to 50, 10 to 60, 10 to 70, 10 to 80, 10 to 90, 10 to 100, 10 to 110, 10 to 120, 10 to 130, 10 to 140, 10 to 150, 10 to 160, 10 to 170, 10 to 180, 10 to 190, 10 to 200, 10 to 210, 10 to 220, 10 to 230, 10 to 240, 10 to 250, 10 to 260, 10 to 270, 10 to 280, 10 to 290, 10 to 300, 10 to 310, 10 to 320, 10 to 330, 10 to 340, 10 to 350, 10 to 360, 10 to 370, 10 to 380, 10 to 390, 10 to 400, 10 to 410, 10 to 420, 10 to 430, 10 to 440, 10 to 450, 10 to 460, 10 to 470, 10 to 480, 10 to 490, or 10 to 500 nucleotides.

A guide-target RNA scaffold is formed upon hybridization of an engineered guide RNA of the present disclosure to a target RNA. As disclosed herein, a bulge refers to the structure substantially formed only upon formation of the guide-target RNA scaffold, where contiguous nucleotides in either the engineered guide RNA or the target RNA are not complementary to their positional counterparts on the opposite strand. The nucleotides in a bulge of the guide RNA can comprise any nucleotide, in any order so long as they are not complementary to their positional counterparts on the target RNA. A bulge can change the secondary or tertiary structure of the guide-target RNA scaffold. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a mismatch. Further, where the number of participating nucleotides on either the guide RNA side or the target RNA side exceeds 4, the resulting structure is no longer considered a bulge, but rather, is considered an internal loop. In some embodiments, the guide-target RNA scaffold of the present disclosure has 2 bulges. In some embodiments, the guide-target RNA scaffold of the present disclosure has 3 bulges. In some embodiments, the guide-target RNA scaffold of the present disclosure has 4 bulges. Thus, a bulge can be a structural feature formed from latent structure provided by an engineered latent guide RNA.

In some embodiments, the presence of a bulge in a guide-target RNA scaffold can position or can help to position ADAR to selectively edit the target A in the target RNA and reduce off-target editing of non-target A(s) in the target RNA. In some embodiments, the presence of a bulge in a guide-target RNA scaffold can recruit or help recruit additional amounts of ADAR. Bulges in guide-target RNA scaffolds disclosed herein can recruit other proteins, such as other RNA editing entities. In some embodiments, a bulge positioned 5′ of the edit site can facilitate base-flipping of the target A to be edited. A bulge can also help confer sequence specificity for the A of the target RNA to be edited, relative to other A(s) present in the target RNA. For example, a bulge can help direct ADAR editing by constraining it in an orientation that yields selective editing of the target A.

A guide-target RNA scaffold is formed upon hybridization of an engineered guide RNA of the present disclosure to a target RNA. A bulge can be a symmetrical bulge or an asymmetrical bulge. A symmetrical bulge is formed when the same number of nucleotides is present on each side of the bulge. For example, a symmetrical bulge in a guide-target RNA scaffold of the present disclosure can have the same number of nucleotides on the engineered guide RNA side and the target RNA side of the guide-target RNA scaffold. A symmetrical bulge of the present disclosure can be formed by 2 nucleotides on the engineered guide RNA side of the guide-target RNA scaffold target and 2 nucleotides on the target RNA side of the guide-target RNA scaffold. A symmetrical bulge of the present disclosure can be formed by 3 nucleotides on the engineered guide RNA side of the guide-target RNA scaffold target and 3 nucleotides on the target RNA side of the guide-target RNA scaffold. A symmetrical bulge of the present disclosure can be formed by 4 nucleotides on the engineered guide RNA side of the guide-target RNA scaffold target and 4 nucleotides on the target RNA side of the guide-target RNA scaffold. Thus, a symmetrical bulge can be a structural feature formed from latent structure provided by an engineered latent guide RNA.

A guide-target RNA scaffold is formed upon hybridization of an engineered guide RNA of the present disclosure to a target RNA. A bulge can be a symmetrical bulge or an asymmetrical bulge. An asymmetrical bulge is formed when a different number of nucleotides is present on each side of the bulge. For example, an asymmetrical bulge in a guide-target RNA scaffold of the present disclosure can have different numbers of nucleotides on the engineered guide RNA side and the target RNA side of the guide-target RNA scaffold. An asymmetrical bulge of the present disclosure can be formed by 0 nucleotides on the engineered guide RNA side of the guide-target RNA scaffold and 1 nucleotide on the target RNA side of the guide-target RNA scaffold. An asymmetrical bulge of the present disclosure can be formed by 0 nucleotides on the target RNA side of the guide-target RNA scaffold and 1 nucleotide on the engineered guide RNA side of the guide-target RNA scaffold. An asymmetrical bulge of the present disclosure can be formed by 0 nucleotides on the engineered guide RNA side of the guide-target RNA scaffold and 2 nucleotides on the target RNA side of the guide-target RNA scaffold. An asymmetrical bulge of the present disclosure can be formed by 0 nucleotides on the target RNA side of the guide-target RNA scaffold and 2 nucleotides on the engineered guide RNA side of the guide-target RNA scaffold. An asymmetrical bulge of the present disclosure can be formed by 0 nucleotides on the engineered guide RNA side of the guide-target RNA scaffold and 3 nucleotides on the target RNA side of the guide-target RNA scaffold. An asymmetrical bulge of the present disclosure can be formed by 0 nucleotides on the target RNA side of the guide-target RNA scaffold and 3 nucleotides on the engineered guide RNA side of the guide-target RNA scaffold. An asymmetrical bulge of the present disclosure can be formed by 0 nucleotides on the engineered guide RNA side of the guide-target RNA scaffold and 4 nucleotides on the target RNA side of the guide-target RNA scaffold. An asymmetrical bulge of the present disclosure can be formed by 0 nucleotides on the target RNA side of the guide-target RNA scaffold and 4 nucleotides on the engineered guide RNA side of the guide-target RNA scaffold. An asymmetrical bulge of the present disclosure can be formed by 1 nucleotide on the engineered guide RNA side of the guide-target RNA scaffold and 2 nucleotides on the target RNA side of the guide-target RNA scaffold. An asymmetrical bulge of the present disclosure can be formed by 1 nucleotide on the target RNA side of the guide-target RNA scaffold and 2 nucleotides on the engineered guide RNA side of the guide-target RNA scaffold. An asymmetrical bulge of the present disclosure can be formed by 1 nucleotide on the engineered guide RNA side of the guide-target RNA scaffold and 3 nucleotides on the target RNA side of the guide-target RNA scaffold. An asymmetrical bulge of the present disclosure can be formed by 1 nucleotide on the target RNA side of the guide-target RNA scaffold and 3 nucleotides on the engineered guide RNA side of the guide-target RNA scaffold. An asymmetrical bulge of the present disclosure can be formed by 1 nucleotide on the engineered guide RNA side of the guide-target RNA scaffold and 4 nucleotides on the target RNA side of the guide-target RNA scaffold. An asymmetrical bulge of the present disclosure can be formed by 1 nucleotide on the target RNA side of the guide-target RNA scaffold and 4 nucleotides on the engineered guide RNA side of the guide-target RNA scaffold. An asymmetrical bulge of the present disclosure can be formed by 2 nucleotides on the engineered guide RNA side of the guide-target RNA scaffold and 3 nucleotides on the target RNA side of the guide-target RNA scaffold. An asymmetrical bulge of the present disclosure can be formed by 2 nucleotides on the target RNA side of the guide-target RNA scaffold and 3 nucleotides on the engineered guide RNA side of the guide-target RNA scaffold. An asymmetrical bulge of the present disclosure can be formed by 2 nucleotides on the engineered guide RNA side of the guide-target RNA scaffold and 4 nucleotides on the target RNA side of the guide-target RNA scaffold. An asymmetrical bulge of the present disclosure can be formed by 2 nucleotides on the target RNA side of the guide-target RNA scaffold and 4 nucleotides on the engineered guide RNA side of the guide-target RNA scaffold. An asymmetrical bulge of the present disclosure can be formed by 3 nucleotides on the engineered guide RNA side of the guide-target RNA scaffold and 4 nucleotides on the target RNA side of the guide-target RNA scaffold. An asymmetrical bulge of the present disclosure can be formed by 3 nucleotides on the target RNA side of the guide-target RNA scaffold and 4 nucleotides on the engineered guide RNA side of the guide-target RNA scaffold. Thus, an asymmetrical bulge can be a structural feature formed from latent structure provided by an engineered latent guide RNA.

In some cases, a structural feature can be an internal loop. As disclosed herein, an internal loop refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. The nucleotides in an internal loop of the guide RNA can comprise any nucleotide, in any order so long as they are not complementary to their positional counterparts on the target RNA. Where the number of participating nucleotides on both the guide RNA side and the target RNA side drops below 5, the resulting structure is no longer considered an internal loop, but rather, is considered a bulge or a mismatch, depending on the size of the structural feature. An internal loop can be a symmetrical internal loop or an asymmetrical internal loop. Internal loops present in the vicinity of the edit site can help with base flipping of the target A in the target RNA to be edited.

One side of the internal loop, either on the target RNA side or the engineered guide RNA side of the guide-target RNA scaffold, can be formed by from 5 to 150 nucleotides. One side of the internal loop can be formed by 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 120, 135, 140, 145, 150, 200, 250, 300, 350, 400, 450, 500, 600, 700, 800, 900, or 1000 nucleotides, or any number of nucleotides therebetween. One side of the internal loop can be formed by 5 nucleotides. One side of the internal loop can be formed by 10 nucleotides. One side of the internal loop can be formed by 15 nucleotides. One side of the internal loop can be formed by 20 nucleotides. One side of the internal loop can be formed by 25 nucleotides. One side of the internal loop can be formed by 30 nucleotides. One side of the internal loop can be formed by 35 nucleotides. One side of the internal loop can be formed by 40 nucleotides. One side of the internal loop can be formed by 45 nucleotides. One side of the internal loop can be formed by 50 nucleotides. One side of the internal loop can be formed by 55 nucleotides. One side of the internal loop can be formed by 60 nucleotides. One side of the internal loop can be formed by 65 nucleotides. One side of the internal loop can be formed by 70 nucleotides. One side of the internal loop can be formed by 75 nucleotides. One side of the internal loop can be formed by 80 nucleotides. One side of the internal loop can be formed by 85 nucleotides. One side of the internal loop can be formed by 90 nucleotides. One side of the internal loop can be formed by 95 nucleotides. One side of the internal loop can be formed by 100 nucleotides. One side of the internal loop can be formed by 110 nucleotides. One side of the internal loop can be formed by 120 nucleotides. One side of the internal loop can be formed by 130 nucleotides. One side of the internal loop can be formed by 140 nucleotides. One side of the internal loop can be formed by 150 nucleotides. One side of the internal loop can be formed by 200 nucleotides. One side of the internal loop can be formed by 250 nucleotides. One side of the internal loop can be formed by 300 nucleotides. One side of the internal loop can be formed by 350 nucleotides. One side of the internal loop can be formed by 400 nucleotides. One side of the internal loop can be formed by 450 nucleotides. One side of the internal loop can be formed by 500 nucleotides. One side of the internal loop can be formed by 600 nucleotides. One side of the internal loop can be formed by 700 nucleotides. One side of the internal loop can be formed by 800 nucleotides. One side of the internal loop can be formed by 900 nucleotides. One side of the internal loop can be formed by 1000 nucleotides. Thus, an internal loop can be a structural feature formed from latent structure provided by an engineered latent guide RNA.

An internal loop can be a symmetrical internal loop or an asymmetrical internal loop. A symmetrical internal loop is formed when the same number of nucleotides is present on each side of the internal loop. For example, a symmetrical internal loop in a guide-target RNA scaffold of the present disclosure can have the same number of nucleotides on the engineered guide RNA side and the target RNA side of the guide-target RNA scaffold. A symmetrical internal loop of the present disclosure can be formed by 5 nucleotides on the engineered guide RNA side of the guide-target RNA scaffold target and 5 nucleotides on the target RNA side of the guide-target RNA scaffold. A symmetrical internal loop of the present disclosure can be formed by 6 nucleotides on the engineered guide RNA side of the guide-target RNA scaffold target and 6 nucleotides on the target RNA side of the guide-target RNA scaffold. A symmetrical internal loop of the present disclosure can be formed by 7 nucleotides on the engineered guide RNA side of the guide-target RNA scaffold target and 7 nucleotides on the target RNA side of the guide-target RNA scaffold. A symmetrical internal loop of the present disclosure can be formed by 8 nucleotides on the engineered guide RNA side of the guide-target RNA scaffold target and 8 nucleotides on the target RNA side of the guide-target RNA scaffold. A symmetrical internal loop of the present disclosure can be formed by 9 nucleotides on the engineered guide RNA side of the guide-target RNA scaffold target and 9 nucleotides on the target RNA side of the guide-target RNA scaffold. A symmetrical internal loop of the present disclosure can be formed by 10 nucleotides on the engineered guide RNA side of the guide-target RNA scaffold target and 10 nucleotides on the target RNA side of the guide-target RNA scaffold. A symmetrical internal loop of the present disclosure can be formed by 11 nucleotides on the engineered guide RNA side of the guide-target RNA scaffold target and 11 nucleotides on the target RNA side of the guide-target RNA scaffold. A symmetrical internal loop of the present disclosure can be formed by 12 nucleotides on the engineered guide RNA side of the guide-target RNA scaffold target and 12 nucleotides on the target RNA side of the guide-target RNA scaffold. A symmetrical internal loop of the present disclosure can be formed by 13 nucleotides on the engineered guide RNA side of the guide-target RNA scaffold target and 13 nucleotides on the target RNA side of the guide-target RNA scaffold. A symmetrical internal loop of the present disclosure can be formed by 14 nucleotides on the engineered guide RNA side of the guide-target RNA scaffold target and 14 nucleotides on the target RNA side of the guide-target RNA scaffold. A symmetrical internal loop of the present disclosure can be formed by 15 nucleotides on the engineered guide RNA side of the guide-target RNA scaffold target and 15 nucleotides on the target RNA side of the guide-target RNA scaffold. A symmetrical internal loop of the present disclosure can be formed by 20 nucleotides on the engineered guide RNA side of the guide-target RNA scaffold target and 20 nucleotides on the target RNA side of the guide-target RNA scaffold. A symmetrical internal loop of the present disclosure can be formed by 30 nucleotides on the engineered guide RNA side of the guide-target RNA scaffold target and 30 nucleotides on the target RNA side of the guide-target RNA scaffold. A symmetrical internal loop of the present disclosure can be formed by 40 nucleotides on the engineered guide RNA side of the guide-target RNA scaffold target and 40 nucleotides on the target RNA side of the guide-target RNA scaffold. A symmetrical internal loop of the present disclosure can be formed by 50 nucleotides on the engineered guide RNA side of the guide-target RNA scaffold target and 50 nucleotides on the target RNA side of the guide-target RNA scaffold. A symmetrical internal loop of the present disclosure can be formed by 60 nucleotides on the engineered guide RNA side of the guide-target RNA scaffold target and 60 nucleotides on the target RNA side of the guide-target RNA scaffold. A symmetrical internal loop of the present disclosure can be formed by 70 nucleotides on the engineered guide RNA side of the guide-target RNA scaffold target and 70 nucleotides on the target RNA side of the guide-target RNA scaffold. A symmetrical internal loop of the present disclosure can be formed by 80 nucleotides on the engineered guide RNA side of the guide-target RNA scaffold target and 80 nucleotides on the target RNA side of the guide-target RNA scaffold. A symmetrical internal loop of the present disclosure can be formed by 90 nucleotides on the engineered guide RNA side of the guide-target RNA scaffold target and 90 nucleotides on the target RNA side of the guide-target RNA scaffold. A symmetrical internal loop of the present disclosure can be formed by 100 nucleotides on the engineered guide RNA side of the guide-target RNA scaffold target and 100 nucleotides on the target RNA side of the guide-target RNA scaffold. A symmetrical internal loop of the present disclosure can be formed by 110 nucleotides on the engineered guide RNA side of the guide-target RNA scaffold target and 110 nucleotides on the target RNA side of the guide-target RNA scaffold. A symmetrical internal loop of the present disclosure can be formed by 120 nucleotides on the engineered guide RNA side of the guide-target RNA scaffold target and 120 nucleotides on the target RNA side of the guide-target RNA scaffold. A symmetrical internal loop of the present disclosure can be formed by 130 nucleotides on the engineered guide RNA side of the guide-target RNA scaffold target and 130 nucleotides on the target RNA side of the guide-target RNA scaffold. A symmetrical internal loop of the present disclosure can be formed by 140 nucleotides on the engineered guide RNA side of the guide-target RNA scaffold target and 140 nucleotides on the target RNA side of the guide-target RNA scaffold. A symmetrical internal loop of the present disclosure can be formed by 150 nucleotides on the engineered guide RNA side of the guide-target RNA scaffold target and 150 nucleotides on the target RNA side of the guide-target RNA scaffold. A symmetrical internal loop of the present disclosure can be formed by 200 nucleotides on the engineered guide RNA side of the guide-target RNA scaffold target and 200 nucleotides on the target RNA side of the guide-target RNA scaffold. A symmetrical internal loop of the present disclosure can be formed by 250 nucleotides on the engineered guide RNA side of the guide-target RNA scaffold target and 250 nucleotides on the target RNA side of the guide-target RNA scaffold. A symmetrical internal loop of the present disclosure can be formed by 300 nucleotides on the engineered guide RNA side of the guide-target RNA scaffold target and 300 nucleotides on the target RNA side of the guide-target RNA scaffold. A symmetrical internal loop of the present disclosure can be formed by 350 nucleotides on the engineered guide RNA side of the guide-target RNA scaffold target and 350 nucleotides on the target RNA side of the guide-target RNA scaffold. A symmetrical internal loop of the present disclosure can be formed by 400 nucleotides on the engineered guide RNA side of the guide-target RNA scaffold target and 400 nucleotides on the target RNA side of the guide-target RNA scaffold. A symmetrical internal loop of the present disclosure can be formed by 450 nucleotides on the engineered guide RNA side of the guide-target RNA scaffold target and 450 nucleotides on the target RNA side of the guide-target RNA scaffold. A symmetrical internal loop of the present disclosure can be formed by 500 nucleotides on the engineered guide RNA side of the guide-target RNA scaffold target and 500 nucleotides on the target RNA side of the guide-target RNA scaffold. A symmetrical internal loop of the present disclosure can be formed by 600 nucleotides on the engineered guide RNA side of the guide-target RNA scaffold target and 600 nucleotides on the target RNA side of the guide-target RNA scaffold. A symmetrical internal loop of the present disclosure can be formed by 700 nucleotides on the engineered guide RNA side of the guide-target RNA scaffold target and 700 nucleotides on the target RNA side of the guide-target RNA scaffold. A symmetrical internal loop of the present disclosure can be formed by 800 nucleotides on the engineered guide RNA side of the guide-target RNA scaffold target and 800 nucleotides on the target RNA side of the guide-target RNA scaffold. A symmetrical internal loop of the present disclosure can be formed by 900 nucleotides on the engineered guide RNA side of the guide-target RNA scaffold target and 900 nucleotides on the target RNA side of the guide-target RNA scaffold. A symmetrical internal loop of the present disclosure can be formed by 1000 nucleotides on the engineered guide RNA side of the guide-target RNA scaffold target and 1000 nucleotides on the target RNA side of the guide-target RNA scaffold. Thus, a symmetrical internal loop can be a structural feature formed from latent structure provided by an engineered latent guide RNA.

In some embodiments, a symmetrical internal loop can be positioned upstream (5′) of the target A (0 position), downstream (3′) of the target A, or both. In some embodiments, when referring to a location of a structural feature a “−” or negative integer indicates a nucleotide upstream (5′) of the target A or of a specified position (e.g., position 0 ATTAAA), while a positive integer indicates a nucleotide downstream (3′) of the target A, or of a specified position. In some instances, a first symmetrical internal loop can be downstream of the target A and a second symmetrical internal loop can be upstream of the target A. In some cases, a symmetric internal loop can be from position: −1 to −25, −2 to −10, −4 to −8, −5 to −7, −2 to −15, −4 to −20, −8 to −15, or −10 to −22 relative to the target A. In some cases, a symmetric internal loop can be located at position: −25, −24, −23, −22, −21, −20, −19, −18, −17, −16, −15, −14, −13, −12, −11, −10, −9, −8, −7, −6, −5, −4, −3, −2, or −1 relative to the target A. In some cases, a symmetric internal loop can be from position: +1 to +60, +10 to +50, +10 to +40, +20 to +50, +20 to +40, +25 to +45, +31 to +35, +10 to +20, +15 to +30, +25 to +45, or +45 to +60 relative to the target A. In some cases, a symmetric internal loop can be located at position: 1, +2, +3, +4, +5, +6, +7, +8, +9, +10, +11, +12, +13, +14, +15, +16, +17, +18, +19, +20, +21, +22, +23, +24, +25, +26, +27, +28, +29, +30, +31, +32, +33, +34, +35, +36, +37, +38, +39, +40, +41, +42, +43, +44, +45, +46, +47, +48, +49, +50, +51, +52, +53, +54, +55, +56, +57, +58, +59, or +60 relative to the target A. In some cases, a first symmetric internal loop within about: 80 bp, 70 bp, 60 bp, 50 bp, 40 bp, 30 bp, 25 bp, 20 bp, 15 bp, 10 bp, or 5 bp of the 5′ end of the guide RNA, and a second symmetric internal loop within about: 80 bp, 70 bp, 60 bp, 50 bp, 40 bp, 30 bp, 25 bp, 20 bp, 15 bp, 10 bp, or 5 bp of the 3′ end of the guide RNA.

An asymmetrical internal loop is formed when a different number of nucleotides is present on each side of the internal loop. For example, an asymmetrical internal loop in a guide-target RNA scaffold of the present disclosure can have different numbers of nucleotides on the engineered guide RNA side and the target RNA side of the guide-target RNA scaffold.

An asymmetrical internal loop of the present disclosure can be formed by from 5 to 150 nucleotides on the engineered guide RNA side of the guide-target RNA scaffold and from 5 to 150 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by from 5 to 1000 nucleotides on the engineered guide RNA side of the guide-target RNA scaffold and from 5 to 1000 nucleotides on the target RNA side of the guide-target RNA scaffold, wherein the number of nucleotides is the different on the engineered side of the guide-target RNA scaffold target than the number of nucleotides on the target RNA side of the guide-target RNA scaffold. An asymmetrical internal loop of the present disclosure can be formed by 5 nucleotides on the engineered guide RNA side of the guide-target RNA scaffold and 6 nucleotides on the target RNA side of the guide-target RNA scaffold. An asymmetrical internal loop of the present disclosure can be formed by 5 nucleotides on the target RNA side of the guide-target RNA scaffold and 6 nucleotides on the engineered guide RNA side of the guide-target RNA scaffold. An asymmetrical internal loop of the present disclosure can be formed by 5 nucleotides on the engineered guide RNA side of the guide-target RNA scaffold and 7 nucleotides on the target RNA side of the guide-target RNA scaffold. An asymmetrical internal loop of the present disclosure can be formed by 5 nucleotides on the target RNA side of the guide-target RNA scaffold and 7 nucleotides on the engineered guide RNA side of the guide-target RNA scaffold. An asymmetrical internal loop of the present disclosure can be formed by 5 nucleotides on the engineered guide RNA side of the guide-target RNA scaffold and 8 nucleotides internal loop the target RNA side of the guide-target RNA scaffold. An asymmetrical internal loop of the present disclosure can be formed by 5 nucleotides on the target RNA side of the guide-target RNA scaffold and 8 nucleotides on the engineered guide RNA side of the guide-target RNA scaffold. An asymmetrical internal loop of the present disclosure can be formed by 5 nucleotides on the engineered guide RNA side of the guide-target RNA scaffold and 9 nucleotides internal loop the target RNA side of the guide-target RNA scaffold. An asymmetrical internal loop of the present disclosure can be formed by 5 nucleotides on the target RNA side of the guide-target RNA scaffold and 9 nucleotides on the engineered guide RNA side of the guide-target RNA scaffold. An asymmetrical internal loop of the present disclosure can be formed by 5 nucleotides on the engineered guide RNA side of the guide-target RNA scaffold and 10 nucleotides internal loop the target RNA side of the guide-target RNA scaffold. An asymmetrical internal loop of the present disclosure can be formed by 5 nucleotides on the target RNA side of the guide-target RNA scaffold and 10 nucleotides on the engineered guide RNA side of the guide-target RNA scaffold. An asymmetrical internal loop of the present disclosure can be formed by 6 nucleotides on the engineered guide RNA side of the guide-target RNA scaffold and 7 nucleotides internal loop the target RNA side of the guide-target RNA scaffold. An asymmetrical internal loop of the present disclosure can be formed by 6 nucleotides on the target RNA side of the guide-target RNA scaffold and 7 nucleotides on the engineered guide RNA side of the guide-target RNA scaffold. An asymmetrical internal loop of the present disclosure can be formed by 6 nucleotides on the engineered guide RNA side of the guide-target RNA scaffold and 8 nucleotides internal loop the target RNA side of the guide-target RNA scaffold. An asymmetrical internal loop of the present disclosure can be formed by 6 nucleotides on the target RNA side of the guide-target RNA scaffold and 8 nucleotides on the engineered guide RNA side of the guide-target RNA scaffold. An asymmetrical internal loop of the present disclosure can be formed by 6 nucleotides on the engineered guide RNA side of the guide-target RNA scaffold and 9 nucleotides internal loop the target RNA side of the guide-target RNA scaffold. An asymmetrical internal loop of the present disclosure can be formed by 6 nucleotides on the target RNA side of the guide-target RNA scaffold and 9 nucleotides on the engineered guide RNA side of the guide-target RNA scaffold. An asymmetrical internal loop of the present disclosure can be formed by 6 nucleotides on the engineered guide RNA side of the guide-target RNA scaffold and 10 nucleotides internal loop the target RNA side of the guide-target RNA scaffold. An asymmetrical internal loop of the present disclosure can be formed by 6 nucleotides on the target RNA side of the guide-target RNA scaffold and 10 nucleotides on the engineered guide RNA side of the guide-target RNA scaffold. An asymmetrical internal loop of the present disclosure can be formed by 7 nucleotides on the engineered guide RNA side of the guide-target RNA scaffold and 8 nucleotides internal loop the target RNA side of the guide-target RNA scaffold. An asymmetrical internal loop of the present disclosure can be formed by 7 nucleotides on the target RNA side of the guide-target RNA scaffold and 8 nucleotides on the engineered guide RNA side of the guide-target RNA scaffold. An asymmetrical internal loop of the present disclosure can be formed by 7 nucleotides on the engineered guide RNA side of the guide-target RNA scaffold and 9 nucleotides internal loop the target RNA side of the guide-target RNA scaffold. An asymmetrical internal loop of the present disclosure can be formed by 7 nucleotides on the target RNA side of the guide-target RNA scaffold and 9 nucleotides on the engineered guide RNA side of the guide-target RNA scaffold. An asymmetrical internal loop of the present disclosure can be formed by 7 nucleotides on the engineered guide RNA side of the guide-target RNA scaffold and 10 nucleotides internal loop the target RNA side of the guide-target RNA scaffold. An asymmetrical internal loop of the present disclosure can be formed by 7 nucleotides on the target RNA side of the guide-target RNA scaffold and 10 nucleotides on the engineered guide RNA side of the guide-target RNA scaffold. An asymmetrical internal loop of the present disclosure can be formed by 8 nucleotides on the engineered guide RNA side of the guide-target RNA scaffold and 9 nucleotides internal loop the target RNA side of the guide-target RNA scaffold. An asymmetrical internal loop of the present disclosure can be formed by 8 nucleotides on the target RNA side of the guide-target RNA scaffold and 9 nucleotides on the engineered guide RNA side of the guide-target RNA scaffold. An asymmetrical internal loop of the present disclosure can be formed by 8 nucleotides on the engineered guide RNA side of the guide-target RNA scaffold and 10 nucleotides internal loop the target RNA side of the guide-target RNA scaffold. An asymmetrical internal loop of the present disclosure can be formed by 8 nucleotides on the target RNA side of the guide-target RNA scaffold and 10 nucleotides on the engineered guide RNA side of the guide-target RNA scaffold. An asymmetrical internal loop of the present disclosure can be formed by 9 nucleotides on the engineered guide RNA side of the guide-target RNA scaffold and 10 nucleotides internal loop the target RNA side of the guide-target RNA scaffold. An asymmetrical internal loop of the present disclosure can be formed by 9 nucleotides on the target RNA side of the guide-target RNA scaffold and 10 nucleotides on the engineered guide RNA side of the guide-target RNA scaffold. An asymmetrical internal loop of the present disclosure can be formed by 5 nucleotides on the target RNA side of the guide-target RNA scaffold and 50 nucleotides on the engineered guide RNA side of the guide-target RNA scaffold. An asymmetrical internal loop of the present disclosure can be formed by 5 nucleotides on the target RNA side of the guide-target RNA scaffold and 100 nucleotides on the engineered guide RNA side of the guide-target RNA scaffold. An asymmetrical internal loop of the present disclosure can be formed by 5 nucleotides on the target RNA side of the guide-target RNA scaffold and 150 nucleotides on the engineered guide RNA side of the guide-target RNA scaffold. An asymmetrical internal loop of the present disclosure can be formed by 5 nucleotides on the target RNA side of the guide-target RNA scaffold and 200 nucleotides on the engineered guide RNA side of the guide-target RNA scaffold. An asymmetrical internal loop of the present disclosure can be formed by 5 nucleotides on the target RNA side of the guide-target RNA scaffold and 300 nucleotides on the engineered guide RNA side of the guide-target RNA scaffold. An asymmetrical internal loop of the present disclosure can be formed by 5 nucleotides on the target RNA side of the guide-target RNA scaffold and 400 nucleotides on the engineered guide RNA side of the guide-target RNA scaffold. An asymmetrical internal loop of the present disclosure can be formed by 5 nucleotides on the target RNA side of the guide-target RNA scaffold and 500 nucleotides on the engineered guide RNA side of the guide-target RNA scaffold. An asymmetrical internal loop of the present disclosure can be formed by 5 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 5 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 5 nucleotides on the engineered guide RNA side of the guide-target RNA scaffold. An asymmetrical internal loop of the present disclosure can be formed by 300 nucleotides on the target RNA side of the guide-target RNA scaffold and 5 nucleotides on the engineered guide RNA side of the guide-target RNA scaffold. An asymmetrical internal loop of the present disclosure can be formed by 200 nucleotides on the target RNA side of the guide-target RNA scaffold and 5 nucleotides on the engineered guide RNA side of the guide-target RNA scaffold. An asymmetrical internal loop of the present disclosure can be formed by 150 nucleotides on the target RNA side of the guide-target RNA scaffold and 5 nucleotides on the engineered guide RNA side of the guide-target RNA scaffold. An asymmetrical internal loop of the present disclosure can be formed by 100 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 5 nucleotides on the engineered guide RNA side of the guide-target RNA scaffold. An asymmetrical internal loop of the present disclosure can be formed by 50 nucleotides on the target RNA side of the guide-target RNA scaffold and 100 nucleotides on the engineered guide RNA side of the guide-target RNA scaffold. An asymmetrical internal loop of the present disclosure can be formed by 50 nucleotides on the target RNA side of the guide-target RNA scaffold and 150 nucleotides on the engineered guide RNA side of the guide-target RNA scaffold. An asymmetrical internal loop of the present disclosure can be formed by 50 nucleotides on the target RNA side of the guide-target RNA scaffold and 200 nucleotides on the engineered guide RNA side of the guide-target RNA scaffold. An asymmetrical internal loop of the present disclosure can be formed by 50 nucleotides on the target RNA side of the guide-target RNA scaffold and 300 nucleotides on the engineered guide RNA side of the guide-target RNA scaffold. An asymmetrical internal loop of the present disclosure can be formed by 50 nucleotides on the target RNA side of the guide-target RNA scaffold and 400 nucleotides on the engineered guide RNA side of the guide-target RNA scaffold. An asymmetrical internal loop of the present disclosure can be formed by 50 nucleotides on the target RNA side of the guide-target RNA scaffold and 500 nucleotides on the engineered guide RNA side of the guide-target RNA scaffold. An asymmetrical internal loop of the present disclosure can be formed by 50 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 50 nucleotides on the engineered guide RNA side of the guide-target RNA scaffold. An asymmetrical internal loop of the present disclosure can be formed by 500 nucleotides on the target RNA side of the guide-target RNA scaffold and 50 nucleotides on the engineered guide RNA side of the guide-target RNA scaffold. An asymmetrical internal loop of the present disclosure can be formed by 400 nucleotides on the target RNA side of the guide-target RNA scaffold and 50 nucleotides on the engineered guide RNA side of the guide-target RNA scaffold. An asymmetrical internal loop of the present disclosure can be formed by 300 nucleotides on the target RNA side of the guide-target RNA scaffold and 50 nucleotides on the engineered guide RNA side of the guide-target RNA scaffold. An asymmetrical internal loop of the present disclosure can be formed by 200 nucleotides on the target RNA side of the guide-target RNA scaffold and 50 nucleotides on the engineered guide RNA side of the guide-target RNA scaffold. An asymmetrical internal loop of the present disclosure can be formed by 150 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 50 nucleotides on the engineered guide RNA side of the guide-target RNA scaffold. An asymmetrical internal loop of the present disclosure can be formed by 100 nucleotides on the target RNA side of the guide-target RNA scaffold and 150 nucleotides on the engineered guide RNA side of the guide-target RNA scaffold. An asymmetrical internal loop of the present disclosure can be formed by 100 nucleotides on the target RNA side of the guide-target RNA scaffold and 200 nucleotides on the engineered guide RNA side of the guide-target RNA scaffold. An asymmetrical internal loop of the present disclosure can be formed by 100 nucleotides on the target RNA side of the guide-target RNA scaffold and 300 nucleotides on the engineered guide RNA side of the guide-target RNA scaffold. An asymmetrical internal loop of the present disclosure can be formed by 100 nucleotides on the target RNA side of the guide-target RNA scaffold and 400 nucleotides on the engineered guide RNA side of the guide-target RNA scaffold. An asymmetrical internal loop of the present disclosure can be formed by 100 nucleotides on the target RNA side of the guide-target RNA scaffold and 500 nucleotides on the engineered guide RNA side of the guide-target RNA scaffold. An asymmetrical internal loop of the present disclosure can be formed by 100 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 100 nucleotides on the engineered guide RNA side of the guide-target RNA scaffold. An asymmetrical internal loop of the present disclosure can be formed by 500 nucleotides on the target RNA side of the guide-target RNA scaffold and 100 nucleotides on the engineered guide RNA side of the guide-target RNA scaffold. An asymmetrical internal loop of the present disclosure can be formed by 400 nucleotides on the target RNA side of the guide-target RNA scaffold and 100 nucleotides on the engineered guide RNA side of the guide-target RNA scaffold. An asymmetrical internal loop of the present disclosure can be formed by 300 nucleotides on the target RNA side of the guide-target RNA scaffold and 100 nucleotides on the engineered guide RNA side of the guide-target RNA scaffold. An asymmetrical internal loop of the present disclosure can be formed by 200 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 100 nucleotides on the engineered guide RNA side of the guide-target RNA scaffold. An asymmetrical internal loop of the present disclosure can be formed by 150 nucleotides on the target RNA side of the guide-target RNA scaffold and 200 nucleotides on the engineered guide RNA side of the guide-target RNA scaffold. An asymmetrical internal loop of the present disclosure can be formed by 150 nucleotides on the target RNA side of the guide-target RNA scaffold and 300 nucleotides on the engineered guide RNA side of the guide-target RNA scaffold. An asymmetrical internal loop of the present disclosure can be formed by 150 nucleotides on the target RNA side of the guide-target RNA scaffold and 400 nucleotides on the engineered guide RNA side of the guide-target RNA scaffold. An asymmetrical internal loop of the present disclosure can be formed by 150 nucleotides on the target RNA side of the guide-target RNA scaffold and 500 nucleotides on the engineered guide RNA side of the guide-target RNA scaffold. An asymmetrical internal loop of the present disclosure can be formed by 150 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 150 nucleotides on the engineered guide RNA side of the guide-target RNA scaffold. An asymmetrical internal loop of the present disclosure can be formed by 500 nucleotides on the target RNA side of the guide-target RNA scaffold and 5 nucleotides on the engineered guide RNA side of the guide-target RNA scaffold. An asymmetrical internal loop of the present disclosure can be formed by 400 nucleotides on the target RNA side of the guide-target RNA scaffold and 150 nucleotides on the engineered guide RNA side of the guide-target RNA scaffold. An asymmetrical internal loop of the present disclosure can be formed by 300 nucleotides on the target RNA side of the guide-target RNA scaffold and 150 nucleotides on the engineered guide RNA side of the guide-target RNA scaffold. An asymmetrical internal loop of the present disclosure can be formed by 200 nucleotides on the target RNA side of the guide-target RNA scaffold and 300 nucleotides on the engineered guide RNA side of the guide-target RNA scaffold. An asymmetrical internal loop of the present disclosure can be formed by 200 nucleotides on the target RNA side of the guide-target RNA scaffold and 400 nucleotides on the engineered guide RNA side of the guide-target RNA scaffold. An asymmetrical internal loop of the present disclosure can be formed by 200 nucleotides on the target RNA side of the guide-target RNA scaffold and 500 nucleotides on the engineered guide RNA side of the guide-target RNA scaffold. An asymmetrical internal loop of the present disclosure can be formed by 200 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 200 nucleotides on the engineered guide RNA side of the guide-target RNA scaffold. An asymmetrical internal loop of the present disclosure can be formed by 500 nucleotides on the target RNA side of the guide-target RNA scaffold and 200 nucleotides on the engineered guide RNA side of the guide-target RNA scaffold. An asymmetrical internal loop of the present disclosure can be formed by 400 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 200 nucleotides on the engineered guide RNA side of the guide-target RNA scaffold. An asymmetrical internal loop of the present disclosure can be formed by 300 nucleotides on the target RNA side of the guide-target RNA scaffold and 400 nucleotides on the engineered guide RNA side of the guide-target RNA scaffold. An asymmetrical internal loop of the present disclosure can be formed by 300 nucleotides on the target RNA side of the guide-target RNA scaffold and 500 nucleotides on the engineered guide RNA side of the guide-target RNA scaffold. An asymmetrical internal loop of the present disclosure can be formed by 300 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 300 nucleotides on the engineered guide RNA side of the guide-target RNA scaffold. An asymmetrical internal loop of the present disclosure can be formed by 500 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 300 nucleotides on the engineered guide RNA side of the guide-target RNA scaffold. An asymmetrical internal loop of the present disclosure can be formed by 400 nucleotides on the target RNA side of the guide-target RNA scaffold and 500 nucleotides on the engineered guide RNA side of the guide-target RNA scaffold. An asymmetrical internal loop of the present disclosure can be formed by 400 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 400 nucleotides on the engineered guide RNA side of the guide-target RNA scaffold. An asymmetrical internal loop of the present disclosure can be formed by 500 nucleotides on the target RNA side of the guide-target RNA scaffold and 1000 nucleotides on the engineered guide RNA side of the guide-target RNA scaffold. An asymmetrical internal loop of the present disclosure can be formed by 1000 nucleotides on the target RNA side of the guide-target RNA scaffold and 500 nucleotides on the engineered guide RNA side of the guide-target RNA scaffold. Thus, an asymmetrical internal loop can be a structural feature formed from latent structure provided by an engineered latent guide RNA.

As disclosed herein, a “base paired (bp) region” refers to a region of the guide-target RNA scaffold in which bases in the guide RNA are paired with opposing bases in the target RNA. Base paired regions can extend from one end or proximal to one end of the guide-target RNA scaffold to or proximal to the other end of the guide-target RNA scaffold. Base paired regions can extend between two structural features. Base paired regions can extend from one end or proximal to one end of the guide-target RNA scaffold to or proximal to a structural feature. Base paired regions can extend from a structural feature to the other end of the guide-target RNA scaffold. In some embodiments, a base paired region has from from 1 bp to 100 bp, from 1 bp to 90 bp, from 1 bp to 80 bp, from 1 bp to 70 bp, from 1 bp to 60 bp, from 1 bp to 50 bp, from 1 bp to 45 bp, from 1 bp to 40 bp, from 1 bp to 35 bp, from 1 bp to 30 bp, from 1 bp to 25 bp, from 1 bp to 20 bp, from 1 bp to 15 bp, from 1 bp to 10 bp, from 1 bp to 5 bp, from 5 bp to 10 bp, from 5 bp to 20 bp, from 10 bp to 20 bp, from 10 bp to 50 bp, from 5 bp to 50 bp, at least 1 bp, at least 2 bp, at least 3 bp, at least 4 bp, at least 5 bp, at least 6 bp, at least 7 bp, at least 8 bp, at least 9 bp, at least 10 bp, at least 12 bp, at least 14 bp, at least 16 bp, at least 18 bp, at least 20 bp, at least 25 bp, at least 30 bp, at least 35 bp, at least 40 bp, at least 45 bp, at least 50 bp, at least 60 bp, at least 70 bp, at least 80 bp, at least 90 bp, at least 100 bp.

In some cases, the structural feature formed upon hybridization of an engineered guide RNA of the present disclosure to a target DUX4 RNA comprises a 1 nucleotide mismatch formed 3 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 8 and, the guide-target RNA scaffold formed upon hybridization of said engineered guide RNA to the target DUX4 RNA comprises a 1 nucleotide mismatch formed 3 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 8 and, the guide-target RNA scaffold formed upon hybridization of said engineered guide RNA to the target DUX4 RNA comprises a 1 nucleotide mismatch formed 3 nucleotides downstream (3′) from the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 1 nucleotide mismatch formed 5 nucleotides downstream (5′) from the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 10 and, the guide-target RNA scaffold formed upon hybridization of said engineered guide RNA to the target DUX4 RNA comprises a 1 nucleotide mismatch formed 5 nucleotides downstream (5′) from the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 10 and, the guide-target RNA scaffold formed upon hybridization of said engineered guide RNA to the target DUX4 RNA comprises a 1 nucleotide mismatch formed 5 nucleotides downstream (5′) from the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 1 nucleotide mismatch formed 5 nucleotides downstream (5′) from the target A, and a 6 nucleotide internal symmetric loop formed 21 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 14 and, the guide-target RNA scaffold formed upon hybridization of said engineered guide RNA to the target DUX4 RNA comprises a 1 nucleotide mismatch formed 5 nucleotides downstream (5′) from the target A, and a 6 nucleotide internal symmetric loop formed 21 nucleotides downstream (3′) from the target A. In some cases, an engineered guide RNA of the present disclosure to a target DUX4 RNA has a sequence of SEQ ID NO: 14 and, the guide-target RNA scaffold formed upon hybridization of said engineered guide RNA to the target DUX4 RNA comprises a 1 nucleotide mismatch formed 5 nucleotides downstream (5′) from the target A, and a 6 nucleotide internal symmetric loop formed 21 nucleotides downstream (3′) from the target A.

In some cases, the structural feature formed upon hybridization of an engineered guide RNA of the present disclosure to a target DUX4 RNA comprises a 6 nucleotide internal symmetric loop formed 21 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 15 and, the guide-target RNA scaffold formed upon hybridization of said engineered guide RNA to the target DUX4 RNA comprises a 6 nucleotide internal symmetric loop formed 21 nucleotides downstream (3′) from the target A. In some cases, an engineered guide RNA of the present disclosure to a target DUX4 RNA has a sequence of SEQ ID NO: 15 and, the guide-target RNA scaffold formed upon hybridization of said engineered guide RNA to the target DUX4 RNA comprises a 6 nucleotide internal symmetric loop formed 21 nucleotides downstream (3′) from the target A.

In some cases, the structural feature formed upon hybridization of an engineered guide RNA of the present disclosure to a target DUX4 RNA comprises a 1 nucleotide mismatch formed 3 nucleotides downstream (5′) from the target A, and a 6 nucleotide internal symmetric loop formed 22 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 17 and, the guide-target RNA scaffold formed upon hybridization of said engineered guide RNA to the target DUX4 RNA comprises a 1 nucleotide mismatch formed 3 nucleotides downstream (5′) from the target A, and a 6 nucleotide internal symmetric loop formed 22 nucleotides downstream (3′) from the target A. In some cases, an engineered guide RNA of the present disclosure to a target DUX4 RNA has a sequence of SEQ ID NO: 17 and, the guide-target RNA scaffold formed upon hybridization of said engineered guide RNA to the target DUX4 RNA comprises a 1 nucleotide mismatch formed 3 nucleotides downstream (5′) from the target A, and a 6 nucleotide internal symmetric loop formed 22 nucleotides downstream (3′) from the target A.

In some cases, the structural feature formed upon hybridization of an engineered guide RNA of the present disclosure to a target DUX4 RNA comprises a 1 nucleotide mismatch formed 5 nucleotides downstream (5′) from the target A, and a 6 nucleotide internal symmetric loop formed 23 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 24 and, the guide-target RNA scaffold formed upon hybridization of said engineered guide RNA to the target DUX4 RNA comprises a 1 nucleotide mismatch formed 5 nucleotides downstream (5′) from the target A, and a 6 nucleotide internal symmetric loop formed 23 nucleotides downstream (3′) from the target A. In some cases, an engineered guide RNA of the present disclosure to a target DUX4 RNA has a sequence of SEQ ID NO: 24 and, the guide-target RNA scaffold formed upon hybridization of said engineered guide RNA to the target DUX4 RNA comprises a 1 nucleotide mismatch formed 5 nucleotides downstream (5′) from the target A, and a 6 nucleotide internal symmetric loop formed 23 nucleotides downstream (3′) from the target A.

In some cases, the structural feature formed upon hybridization of an engineered guide RNA of the present disclosure to a target DUX4 RNA comprises a 1 nucleotide mismatch formed 3 nucleotides downstream (5′) from the target A, and a 6 nucleotide internal symmetric loop formed 33 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 72 and, the guide-target RNA scaffold formed upon hybridization of said engineered guide RNA to the target DUX4 RNA comprises a 1 nucleotide mismatch formed 3 nucleotides downstream (5′) from the target A, and a 6 nucleotide internal symmetric loop formed 33 nucleotides downstream (3′) from the target A. In some cases, an engineered guide RNA of the present disclosure to a target DUX4 RNA has a sequence of SEQ ID NO: 72 and, the guide-target RNA scaffold formed upon hybridization of said engineered guide RNA to the target DUX4 RNA comprises a 1 nucleotide mismatch formed 3 nucleotides downstream (5′) from the target A, and a 6 nucleotide internal symmetric loop formed 33 nucleotides downstream (3′) from the target A.

In some cases, the structural feature formed upon hybridization of an engineered guide RNA of the present disclosure to a target DUX4 RNA comprises a 6 nucleotide internal symmetric loop formed 12 nucleotides upstream (5′) from the target A, a 1 nucleotide mismatch formed 5 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 195 and, the guide-target RNA scaffold formed upon hybridization of said engineered guide RNA to the target DUX4 RNA comprises a 6 nucleotide internal symmetric loop formed 12 nucleotides upstream (5′) from the target A, a 1 nucleotide mismatch formed 5 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has a sequence of SEQ ID NO: 195 and, the guide-target RNA scaffold formed upon hybridization of said engineered guide RNA to the target DUX4 RNA comprises a 6 nucleotide internal symmetric loop formed 12 nucleotides upstream (5′) from the target A, a 1 nucleotide mismatch formed 5 nucleotides downstream (3′) from the target A, and a 6 nucleotide internal symmetric loop formed 33 nucleotides downstream (3′) from the target A.

In some cases, the structural feature formed upon hybridization of an engineered guide RNA of the present disclosure to a target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 252 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 252 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 28 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 291 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 28 nucleotides downstream (3′) from the target A. In some cases, an engineered guide RNA of the present disclosure to a target DUX4 RNA has a sequence of SEQ ID NO: 291 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 28 nucleotides downstream (3′) from the target A.

In some cases, the structural feature formed upon hybridization of an engineered guide RNA of the present disclosure to a target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 41 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 352 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 41 nucleotides downstream (3′) from the target A. In some cases, an engineered guide RNA of the present disclosure to a target DUX4 RNA has a sequence of SEQ ID NO: 352 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 41 nucleotides downstream (3′) from the target A.

In some cases, the structural feature formed upon hybridization of an engineered guide RNA of the present disclosure to a target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 356 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 356 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 11 nucleotides upstream (5′) from the target A, a 1 nucleotide mismatch formed 4 nucleotides downstream (3′) from the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 358 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed 4 nucleotides downstream (3′) from the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 358 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed 4 nucleotides downstream (3′) from the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 44 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 365 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 44 nucleotides downstream (3′) from the target A. In some cases, an engineered guide RNA of the present disclosure to a target DUX4 RNA has a sequence of SEQ ID NO: 365 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed at the target A, and a 6 nucleotide internal symmetric loop formed 44 nucleotides downstream (3′) from the target A.

In some cases, the structural feature formed upon hybridization of an engineered guide RNA of the present disclosure to a target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 375 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 375 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 11 nucleotides upstream (5′) from the target A, a 1 nucleotide mismatch formed 5 nucleotides downstream (3′) from the target A, and a 6 nucleotide internal symmetric loop formed 23 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 392 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed 5 nucleotides downstream (3′) from the target A, and a 6 nucleotide internal symmetric loop formed 23 nucleotides downstream (3′) from the target A. In some cases, an engineered guide RNA of the present disclosure to a target DUX4 RNA has a sequence of SEQ ID NO: 392 and, the guide-target RNA scaffold formed upon hybridization of said engineered guide RNA to the target DUX4 RNA comprises a 6 nucleotide internal symmetric loop formed 11 nucleotides upstream (5′) from the target A, a 1 nucleotide mismatch formed 5 nucleotides downstream (3′) from the target A, and a 6 nucleotide internal symmetric loop formed 23 nucleotides downstream (3′) from the target A.

In some cases, the structural feature formed upon hybridization of an engineered guide RNA of the present disclosure to a target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 24 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 394 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 24 nucleotides downstream (3′) from the target A. In some cases, an engineered guide RNA of the present disclosure to a target DUX4 RNA has a sequence of SEQ ID NO: 394 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 24 nucleotides downstream (3′) from the target A.

In some cases, the structural feature formed upon hybridization of an engineered guide RNA of the present disclosure to a target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 27 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 408 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 27 nucleotides downstream (3′) from the target A. In some cases, an engineered guide RNA of the present disclosure to a target DUX4 RNA has a sequence of SEQ ID NO: 408 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 27 nucleotides downstream (3′) from the target A.

In some cases, the structural feature formed upon hybridization of an engineered guide RNA of the present disclosure to a target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 482 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 482 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 10 nucleotides upstream (5′) from the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 486 and, the guide-target RNA scaffold formed upon hybridization of said engineered guide RNA to the target DUX4 RNA comprises a 6 nucleotide internal symmetric loop formed 10 nucleotides upstream (5′) from the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 486 and, the guide-target RNA scaffold formed upon hybridization of said engineered guide RNA to the target DUX4 RNA comprises a 6 nucleotide internal symmetric loop formed 10 nucleotides upstream (5′) from the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 43 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 487 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 43 nucleotides downstream (3′) from the target A. In some cases, an engineered guide RNA of the present disclosure to a target DUX4 RNA has a sequence of SEQ ID NO: 487 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed at the target A, and a 6 nucleotide internal symmetric loop formed 43 nucleotides downstream (3′) from the target A.

In some cases, the structural feature formed upon hybridization of an engineered guide RNA of the present disclosure to a target DUX4 RNA comprises a 6 nucleotide internal symmetric loop formed 10 nucleotides upstream (5′) from the target A, a 1 nucleotide mismatch formed 4 nucleotides downstream (3′) from the target A, and a 6 nucleotide internal symmetric loop formed 44 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 494 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed 4 nucleotides downstream (3′) from the target A, and a 6 nucleotide internal symmetric loop formed 44 nucleotides downstream (3′) from the target A. In some cases, an engineered guide RNA of the present disclosure to a target DUX4 RNA has a sequence of SEQ ID NO: 494 and, the guide-target RNA scaffold formed upon hybridization of said engineered guide RNA to the target DUX4 RNA comprises a 6 nucleotide internal symmetric loop formed 10 nucleotides upstream (5′) from the target A, a 1 nucleotide mismatch formed 4 nucleotides downstream (3′) from the target A, and a 6 nucleotide internal symmetric loop formed 44 nucleotides downstream (3′) from the target A.

In some cases, the structural feature formed upon hybridization of an engineered guide RNA of the present disclosure to a target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 502 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 502 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 9 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 505 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 505 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 22 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 512 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 22 nucleotides downstream (3′) from the target A. In some cases, an engineered guide RNA of the present disclosure to a target DUX4 RNA has a sequence of SEQ ID NO: 512 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 22 nucleotides downstream (3′) from the target A.

In some cases, the structural feature formed upon hybridization of an engineered guide RNA of the present disclosure to a target DUX4 RNA comprises a 6 nucleotide internal symmetric loop formed 9 nucleotides upstream (5′) from the target A, a 1 nucleotide mismatch formed 3 nucleotides downstream (3′) from the target A, and a 6 nucleotide internal symmetric loop formed 34 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 566 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed 3 nucleotides downstream (3′) from the target A, and a 6 nucleotide internal symmetric loop formed 34 nucleotides downstream (3′) from the target A. In some cases, an engineered guide RNA of the present disclosure to a target DUX4 RNA has a sequence of SEQ ID NO: 566 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed 3 nucleotides downstream (3′) from the target A, and a 6 nucleotide internal symmetric loop formed 34 nucleotides downstream (3′) from the target A.

In some cases, the structural feature formed upon hybridization of an engineered guide RNA of the present disclosure to a target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 40 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 593 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 40 nucleotides downstream (3′) from the target A. In some cases, an engineered guide RNA of the present disclosure to a target DUX4 RNA has a sequence of SEQ ID NO: 593 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed at the target A, and a 6 nucleotide internal symmetric loop formed 40 nucleotides downstream (3′) from the target A.

In some cases, the structural feature formed upon hybridization of an engineered guide RNA of the present disclosure to a target DUX4 RNA comprises a 6 nucleotide internal symmetric loop formed 9 nucleotides upstream (5′) from the target A, a 1 nucleotide mismatch formed 3 nucleotides downstream (3′) from the target A, and a 6 nucleotide internal symmetric loop formed 40 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 594 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed 3 nucleotides downstream (3′) from the target A, and a 6 nucleotide internal symmetric loop formed 40 nucleotides downstream (3′) from the target A. In some cases, an engineered guide RNA of the present disclosure to a target DUX4 RNA has a sequence of SEQ ID NO: 594 and, the guide-target RNA scaffold formed upon hybridization of said engineered guide RNA to the target DUX4 RNA comprises a 6 nucleotide internal symmetric loop formed 9 nucleotides upstream (5′) from the target A, a 1 nucleotide mismatch formed 3 nucleotides downstream (3′) from the target A, and a 6 nucleotide internal symmetric loop formed 40 nucleotides downstream (3′) from the target A.

In some cases, the structural feature formed upon hybridization of an engineered guide RNA of the present disclosure to a target DUX4 RNA comprises a 6 nucleotide internal symmetric loop formed 9 nucleotides upstream (5′) from the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 606 and, the guide-target RNA scaffold formed upon hybridization of said engineered guide RNA to the target DUX4 RNA comprises a 6 nucleotide internal symmetric loop formed 9 nucleotides upstream (5′) from the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 606 and, the guide-target RNA scaffold formed upon hybridization of said engineered guide RNA to the target DUX4 RNA comprises a 6 nucleotide internal symmetric loop formed 9 nucleotides upstream (5′) from the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 8 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 625 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 625 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 8 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 635 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A. In some cases, an engineered guide RNA of the present disclosure to a target DUX4 RNA has a sequence of SEQ ID NO: 635 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A.

In some cases, the structural feature formed upon hybridization of an engineered guide RNA of the present disclosure to a target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 24 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 642 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 24 nucleotides downstream (3′) from the target A. In some cases, an engineered guide RNA of the present disclosure to a target DUX4 RNA has a sequence of SEQ ID NO: 642 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 24 nucleotides downstream (3′) from the target A.

In some cases, the structural feature formed upon hybridization of an engineered guide RNA of the present disclosure to a target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 32 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 679 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 32 nucleotides downstream (3′) from the target A. In some cases, an engineered guide RNA of the present disclosure to a target DUX4 RNA has a sequence of SEQ ID NO: 679 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 32 nucleotides downstream (3′) from the target A.

In some cases, the structural feature formed upon hybridization of an engineered guide RNA of the present disclosure to a target DUX4 RNA comprises a 6 nucleotide internal symmetric loop formed 8 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 680 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has a sequence of SEQ ID NO: 680 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A.

In some cases, the structural feature formed upon hybridization of an engineered guide RNA of the present disclosure to a target DUX4 RNA comprises a 6 nucleotide internal symmetric loop formed 8 nucleotides upstream (5′) from the target A, a 1 nucleotide mismatch formed 3 nucleotides downstream (3′) from the target A, and a 6 nucleotide internal symmetric loop formed 35 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 694 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed 3 nucleotides downstream (3′) from the target A, and a 6 nucleotide internal symmetric loop formed 35 nucleotides downstream (3′) from the target A. In some cases, an engineered guide RNA of the present disclosure to a target DUX4 RNA has a sequence of SEQ ID NO: 694 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed 3 nucleotides downstream (3′) from the target A, and a 6 nucleotide internal symmetric loop formed 35 nucleotides downstream (3′) from the target A.

In some cases, the structural feature formed upon hybridization of an engineered guide RNA of the present disclosure to a target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 727 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 727 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 44 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 737 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 44 nucleotides downstream (3′) from the target A. In some cases, an engineered guide RNA of the present disclosure to a target DUX4 RNA has a sequence of SEQ ID NO: 737 and, the guide-target RNA scaffold formed upon hybridization of said engineered guide RNA to the target DUX4 RNA comprises a 6 nucleotide internal symmetric loop formed 8 nucleotides upstream (5′) from the target A, a 1 nucleotide mismatch formed at the target A, and a 6 nucleotide internal symmetric loop formed 44 nucleotides downstream (3′) from the target A.

In some cases, the structural feature formed upon hybridization of an engineered guide RNA of the present disclosure to a target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 747 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 747 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 7 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 748 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 748 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 7 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 757 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A. In some cases, an engineered guide RNA of the present disclosure to a target DUX4 RNA has a sequence of SEQ ID NO: 757 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A.

In some cases, the structural feature formed upon hybridization of an engineered guide RNA of the present disclosure to a target DUX4 RNA comprises a 6 nucleotide internal symmetric loop formed 7 nucleotides upstream (5′) from the target A, a 1 nucleotide mismatch formed at the target A, and a 6 nucleotide internal symmetric loop formed 25 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 769 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed at the target A, and a 6 nucleotide internal symmetric loop formed 25 nucleotides downstream (3′) from the target A. In some cases, an engineered guide RNA of the present disclosure to a target DUX4 RNA has a sequence of SEQ ID NO: 769 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed at the target A, and a 6 nucleotide internal symmetric loop formed 25 nucleotides downstream (3′) from the target A.

In some cases, the structural feature formed upon hybridization of an engineered guide RNA of the present disclosure to a target DUX4 RNA comprises a 6 nucleotide internal symmetric loop formed 7 nucleotides upstream (5′) from the target A, a 1 nucleotide mismatch formed 4 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 806 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 4 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has a sequence of SEQ ID NO: 806 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 4 nucleotides downstream (3′) from the target A, and a 6 nucleotide internal symmetric loop formed 32 nucleotides downstream (3′) from the target A.

In some cases, the structural feature formed upon hybridization of an engineered guide RNA of the present disclosure to a target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 810 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has a sequence of SEQ ID NO: 810 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A.

In some cases, the structural feature formed upon hybridization of an engineered guide RNA of the present disclosure to a target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 34 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 815 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 34 nucleotides downstream (3′) from the target A. In some cases, an engineered guide RNA of the present disclosure to a target DUX4 RNA has a sequence of SEQ ID NO: 815 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed 3 nucleotides downstream (3′) from the target A, and a 6 nucleotide internal symmetric loop formed 34 nucleotides downstream (3′) from the target A.

In some cases, the structural feature formed upon hybridization of an engineered guide RNA of the present disclosure to a target DUX4 RNA comprises a 6 nucleotide internal symmetric loop formed 7 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 851 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 851 and, the guide-target RNA scaffold formed upon hybridization of said engineered guide RNA to the target DUX4 RNA comprises a 6 nucleotide internal symmetric loop formed 7 nucleotides upstream (5′) from the target A, a 1 nucleotide mismatch formed at the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 871 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 871 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 4 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 873 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 4 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 873 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 4 nucleotides downstream (3′) from the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 874 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A. In some cases, an engineered guide RNA of the present disclosure to a target DUX4 RNA has a sequence of SEQ ID NO: 874 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 5 nucleotides downstream (3′) from the target A, and a 6 nucleotide internal symmetric loop formed 20 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, and a 6 nucleotide internal symmetric loop formed 21 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 880 and, the guide-target RNA scaffold formed upon hybridization of said engineered guide RNA to the target DUX4 RNA comprises a 6 nucleotide internal symmetric loop formed 6 nucleotides upstream (5′) from the target A, and a 6 nucleotide internal symmetric loop formed 21 nucleotides downstream (3′) from the target A. In some cases, an engineered guide RNA of the present disclosure to a target DUX4 RNA has a sequence of SEQ ID NO: 880 and, the guide-target RNA scaffold formed upon hybridization of said engineered guide RNA to the target DUX4 RNA comprises a 6 nucleotide internal symmetric loop formed 6 nucleotides upstream (5′) from the target A, and a 6 nucleotide internal symmetric loop formed 21 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 884 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A. In some cases, an engineered guide RNA of the present disclosure to a target DUX4 RNA has a sequence of SEQ ID NO: 884 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 24 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 892 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 24 nucleotides downstream (3′) from the target A. In some cases, an engineered guide RNA of the present disclosure to a target DUX4 RNA has a sequence of SEQ ID NO: 892 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 24 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 27 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 906 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 27 nucleotides downstream (3′) from the target A. In some cases, an engineered guide RNA of the present disclosure to a target DUX4 RNA has a sequence of SEQ ID NO: 906 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 27 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 930 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has a sequence of SEQ ID NO: 930 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 33 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 934 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 33 nucleotides downstream (3′) from the target A. In some cases, an engineered guide RNA of the present disclosure to a target DUX4 RNA has a sequence of SEQ ID NO: 934 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 33 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 935 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has a sequence of SEQ ID NO: 935 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 40 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 937 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 40 nucleotides downstream (3′) from the target A. In some cases, an engineered guide RNA of the present disclosure to a target DUX4 RNA has a sequence of SEQ ID NO: 937 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 40 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 35 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 944 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 35 nucleotides downstream (3′) from the target A. In some cases, an engineered guide RNA of the present disclosure to a target DUX4 RNA has a sequence of SEQ ID NO: 944 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 35 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 40 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 967 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 40 nucleotides downstream (3′) from the target A. In some cases, an engineered guide RNA of the present disclosure to a target DUX4 RNA has a sequence of SEQ ID NO: 967 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 40 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 976 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 976 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 977 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 977 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 44 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 985 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 44 nucleotides downstream (3′) from the target A. In some cases, an engineered guide RNA of the present disclosure to a target DUX4 RNA has a sequence of SEQ ID NO: 985 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed at the target A, and a 6 nucleotide internal symmetric loop formed 44 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1002 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A. In some cases, an engineered guide RNA of the present disclosure to a target DUX4 RNA has a sequence of SEQ ID NO: 1002 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 5 nucleotides downstream (3′) from the target A, and a 6 nucleotide internal symmetric loop formed 22 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 24 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1008 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 24 nucleotides downstream (3′) from the target A. In some cases, an engineered guide RNA of the present disclosure to a target DUX4 RNA has a sequence of SEQ ID NO: 1008 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 24 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1051 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has a sequence of SEQ ID NO: 1051 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, and a 6 nucleotide internal symmetric loop formed 33 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1054 and, the guide-target RNA scaffold formed upon hybridization of said engineered guide RNA to the target DUX4 RNA comprises a 6 nucleotide internal symmetric loop formed 5 nucleotides upstream (5′) from the target A, and a 6 nucleotide internal symmetric loop formed 33 nucleotides downstream (3′) from the target A. In some cases, an engineered guide RNA of the present disclosure to a target DUX4 RNA has a sequence of SEQ ID NO: 1054 and, the guide-target RNA scaffold formed upon hybridization of said engineered guide RNA to the target DUX4 RNA comprises a 6 nucleotide internal symmetric loop formed 5 nucleotides upstream (5′) from the target A, and a 6 nucleotide internal symmetric loop formed 33 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, a 1 nucleotide mismatch formed 5 nucleotides downstream (3′) from the target A, and a 6 nucleotide internal symmetric loop formed 34 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1058 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 5 nucleotides downstream (3′) from the target A, and a 6 nucleotide internal symmetric loop formed 34 nucleotides downstream (3′) from the target A. In some cases, an engineered guide RNA of the present disclosure to a target DUX4 RNA has a sequence of SEQ ID NO: 1058 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 5 nucleotides downstream (3′) from the target A, and a 6 nucleotide internal symmetric loop formed 34 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, and a 6 nucleotide internal symmetric loop formed 34 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1059 and, the guide-target RNA scaffold formed upon hybridization of said engineered guide RNA to the target DUX4 RNA comprises a 6 nucleotide internal symmetric loop formed 5 nucleotides upstream (5′) from the target A, and a 6 nucleotide internal symmetric loop formed 34 nucleotides downstream (3′) from the target A. In some cases, an engineered guide RNA of the present disclosure to a target DUX4 RNA has a sequence of SEQ ID NO: 1059 and, the guide-target RNA scaffold formed upon hybridization of said engineered guide RNA to the target DUX4 RNA comprises a 6 nucleotide internal symmetric loop formed 5 nucleotides upstream (5′) from the target A, and a 6 nucleotide internal symmetric loop formed 34 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1066 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has a sequence of SEQ ID NO: 1066 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 43 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1098 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 43 nucleotides downstream (3′) from the target A. In some cases, an engineered guide RNA of the present disclosure to a target DUX4 RNA has a sequence of SEQ ID NO: 1098 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 43 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 44 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1103 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 44 nucleotides downstream (3′) from the target A. In some cases, an engineered guide RNA of the present disclosure to a target DUX4 RNA has a sequence of SEQ ID NO: 1103 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed at the target A, and a 6 nucleotide internal symmetric loop formed 44 nucleotides downstream (3′) from the target A.

In some cases, the structural feature formed upon hybridization of an engineered guide RNA of the present disclosure to a target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 44 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1104 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 44 nucleotides downstream (3′) from the target A. In some cases, an engineered guide RNA of the present disclosure to a target DUX4 RNA has a sequence of SEQ ID NO: 1104 and, the guide-target RNA scaffold formed upon hybridization of said engineered guide RNA to the target DUX4 RNA comprises a 6 nucleotide internal symmetric loop formed 5 nucleotides upstream (5′) from the target A, a 1 nucleotide mismatch formed 3 nucleotides downstream (3′) from the target A, and a 6 nucleotide internal symmetric loop formed 44 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, and a 6 nucleotide internal symmetric loop formed 22 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1116 and, the guide-target RNA scaffold formed upon hybridization of said engineered guide RNA to the target DUX4 RNA comprises a 6 nucleotide internal symmetric loop formed 4 nucleotides upstream (5′) from the target A, and a 6 nucleotide internal symmetric loop formed 22 nucleotides downstream (3′) from the target A. In some cases, an engineered guide RNA of the present disclosure to a target DUX4 RNA has a sequence of SEQ ID NO: 1116 and, the guide-target RNA scaffold formed upon hybridization of said engineered guide RNA to the target DUX4 RNA comprises a 6 nucleotide internal symmetric loop formed 4 nucleotides upstream (5′) from the target A, and a 6 nucleotide internal symmetric loop formed 22 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, a 1 nucleotide mismatch formed at the target A, and a 6 nucleotide internal symmetric loop formed 23 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1117 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed at the target A, and a 6 nucleotide internal symmetric loop formed 23 nucleotides downstream (3′) from the target A. In some cases, an engineered guide RNA of the present disclosure to a target DUX4 RNA has a sequence of SEQ ID NO: 1117 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed at the target A, and a 6 nucleotide internal symmetric loop formed 23 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1163 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has a sequence of SEQ ID NO: 1163 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 32 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1168 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A. In some cases, an engineered guide RNA of the present disclosure to a target DUX4 RNA has a sequence of SEQ ID NO: 1168 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 33 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1183 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has a sequence of SEQ ID NO: 1183 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, a 1 nucleotide mismatch formed 5 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1185 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 5 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has a sequence of SEQ ID NO: 1185 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 5 nucleotides downstream (3′) from the target A, and a 6 nucleotide internal symmetric loop formed 36 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, a 1 nucleotide mismatch formed 4 nucleotides downstream (3′) from the target A, and a 6 nucleotide internal symmetric loop formed 38 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1193 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 4 nucleotides downstream (3′) from the target A, and a 6 nucleotide internal symmetric loop formed 38 nucleotides downstream (3′) from the target A. In some cases, an engineered guide RNA of the present disclosure to a target DUX4 RNA has a sequence of SEQ ID NO: 1193 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 4 nucleotides downstream (3′) from the target A, and a 6 nucleotide internal symmetric loop formed 38 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1211 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed at the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 1211 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed at the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1212 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 1212 and, the guide-target RNA scaffold formed upon hybridization of said engineered guide RNA to the target DUX4 RNA comprises a 6 nucleotide internal symmetric loop formed 4 nucleotides upstream (5′) from the target A, a 1 nucleotide mismatch formed 3 nucleotides downstream (3′) from the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 3 nucleotides upstream (5′) from the target A, a 1 nucleotide mismatch formed at the target A, and a 6 nucleotide internal symmetric loop formed 24 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1236 and, the guide-target RNA scaffold formed upon hybridization of said engineered guide RNA to the target DUX4 RNA comprises a 6 nucleotide internal symmetric loop formed 3 nucleotides upstream (5′) from the target A, a 1 nucleotide mismatch formed at the target A, and a 6 nucleotide internal symmetric loop formed 24 nucleotides downstream (3′) from the target A. In some cases, an engineered guide RNA of the present disclosure to a target DUX4 RNA has a sequence of SEQ ID NO: 1236 and, the guide-target RNA scaffold formed upon hybridization of said engineered guide RNA to the target DUX4 RNA comprises a 6 nucleotide internal symmetric loop formed 3 nucleotides upstream (5′) from the target A, a 1 nucleotide mismatch formed at the target A, and a 6 nucleotide internal symmetric loop formed 24 nucleotides downstream (3′) from the target A.

In some cases, the structural feature formed upon hybridization of an engineered guide RNA of the present disclosure to a target DUX4 RNA comprises a 6 nucleotide internal symmetric loop formed 3 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1293 and, the guide-target RNA scaffold formed upon hybridization of said engineered guide RNA to the target DUX4 RNA comprises a 6 nucleotide internal symmetric loop formed 3 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has a sequence of SEQ ID NO: 1293 and, the guide-target RNA scaffold formed upon hybridization of said engineered guide RNA to the target DUX4 RNA comprises a 6 nucleotide internal symmetric loop formed 3 nucleotides upstream (5′) from the target A, a 1 nucleotide mismatch formed 3 nucleotides downstream (3′) from the target A, and a 6 nucleotide internal symmetric loop formed 36 nucleotides downstream (3′) from the target A.

In some cases, the structural feature formed upon hybridization of an engineered guide RNA of the present disclosure to a target DUX4 RNA comprises a 6 nucleotide internal symmetric loop formed 3 nucleotides upstream (5′) from the target A, a 1 nucleotide mismatch formed 4 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1294 and, the guide-target RNA scaffold formed upon hybridization of said engineered guide RNA to the target DUX4 RNA comprises a 6 nucleotide internal symmetric loop formed 3 nucleotides upstream (5′) from the target A, a 1 nucleotide mismatch formed 4 nucleotides downstream (3′) from the target A, and a 6 nucleotide internal symmetric loop formed 36 nucleotides downstream (3′) from the target A. In some cases, an engineered guide RNA of the present disclosure to a target DUX4 RNA has a sequence of SEQ ID NO: 1294 and, the guide-target RNA scaffold formed upon hybridization of said engineered guide RNA to the target DUX4 RNA comprises a 6 nucleotide internal symmetric loop formed 3 nucleotides upstream (5′) from the target A, a 1 nucleotide mismatch formed 4 nucleotides downstream (3′) from the target A, and a 6 nucleotide internal symmetric loop formed 36 nucleotides downstream (3′) from the target A.

In some cases, the structural feature formed upon hybridization of an engineered guide RNA of the present disclosure to a target DUX4 RNA comprises a 6 nucleotide internal symmetric loop formed 3 nucleotides upstream (5′) from the target A, and a 6 nucleotide internal symmetric loop formed 36 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1296 and, the guide-target RNA scaffold formed upon hybridization of said engineered guide RNA to the target DUX4 RNA comprises a 6 nucleotide internal symmetric loop formed 3 nucleotides upstream (5′) from the target A, and a 6 nucleotide internal symmetric loop formed 36 nucleotides downstream (3′) from the target A. In some cases, an engineered guide RNA of the present disclosure to a target DUX4 RNA has a sequence of SEQ ID NO: 1296 and, the guide-target RNA scaffold formed upon hybridization of said engineered guide RNA to the target DUX4 RNA comprises a 6 nucleotide internal symmetric loop formed 3 nucleotides upstream (5′) from the target A, and a 6 nucleotide internal symmetric loop formed 36 nucleotides downstream (3′) from the target A.

In some cases, the structural feature formed upon hybridization of an engineered guide RNA of the present disclosure to a target DUX4 RNA comprises a 6 nucleotide internal symmetric loop formed 2 nucleotides upstream (5′) from the target A, and a 6 nucleotide internal symmetric loop formed 32 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1374 and, the guide-target RNA scaffold formed upon hybridization of said engineered guide RNA to the target DUX4 RNA comprises a 6 nucleotide internal symmetric loop formed 2 nucleotides upstream (5′) from the target A, and a 6 nucleotide internal symmetric loop formed 32 nucleotides downstream (3′) from the target A. In some cases, an engineered guide RNA of the present disclosure to a target DUX4 RNA has a sequence of SEQ ID NO: 1374 and, the guide-target RNA scaffold formed upon hybridization of said engineered guide RNA to the target DUX4 RNA comprises a 6 nucleotide internal symmetric loop formed 2 nucleotides upstream (5′) from the target A, and a 6 nucleotide internal symmetric loop formed 32 nucleotides downstream (3′) from the target A.

In some cases, the structural feature formed upon hybridization of an engineered guide RNA of the present disclosure to a target DUX4 RNA comprises a 6 nucleotide internal symmetric loop formed 2 nucleotides upstream (5′) from the target A, a 1 nucleotide mismatch formed 3 nucleotides downstream (3′) from the target A, and a 6 nucleotide internal symmetric loop formed 37 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1391 and, the guide-target RNA scaffold formed upon hybridization of said engineered guide RNA to the target DUX4 RNA comprises a 6 nucleotide internal symmetric loop formed 2 nucleotides upstream (5′) from the target A, a 1 nucleotide mismatch formed 3 nucleotides downstream (3′) from the target A, and a 6 nucleotide internal symmetric loop formed 37 nucleotides downstream (3′) from the target A. In some cases, an engineered guide RNA of the present disclosure to a target DUX4 RNA has a sequence of SEQ ID NO: 1391 and, the guide-target RNA scaffold formed upon hybridization of said engineered guide RNA to the target DUX4 RNA comprises a 6 nucleotide internal symmetric loop formed 2 nucleotides upstream (5′) from the target A, a 1 nucleotide mismatch formed 3 nucleotides downstream (3′) from the target A, and a 6 nucleotide internal symmetric loop formed 37 nucleotides downstream (3′) from the target A.

In some cases, the structural feature formed upon hybridization of an engineered guide RNA of the present disclosure to a target DUX4 RNA comprises a 6 nucleotide internal symmetric loop formed 2 nucleotides upstream (5′) from the target A, a 1 nucleotide mismatch formed 5 nucleotides downstream (3′) from the target A, and a 6 nucleotide internal symmetric loop formed 42 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1411 and, the guide-target RNA scaffold formed upon hybridization of said engineered guide RNA to the target DUX4 RNA comprises a 6 nucleotide internal symmetric loop formed 2 nucleotides upstream (5′) from the target A, a 1 nucleotide mismatch formed 5 nucleotides downstream (3′) from the target A, and a 6 nucleotide internal symmetric loop formed 42 nucleotides downstream (3′) from the target A. In some cases, an engineered guide RNA of the present disclosure to a target DUX4 RNA has a sequence of SEQ ID NO: 1411 and, the guide-target RNA scaffold formed upon hybridization of said engineered guide RNA to the target DUX4 RNA comprises a 6 nucleotide internal symmetric loop formed 2 nucleotides upstream (5′) from the target A, a 1 nucleotide mismatch formed 5 nucleotides downstream (3′) from the target A, and a 6 nucleotide internal symmetric loop formed 42 nucleotides downstream (3′) from the target A.

In some cases, the structural feature formed upon hybridization of an engineered guide RNA of the present disclosure to a target DUX4 RNA comprises a 6 nucleotide internal symmetric loop formed 1 nucleotide upstream (5′) from the target A, a 1 nucleotide mismatch formed 4 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1463 and, the guide-target RNA scaffold formed upon hybridization of said engineered guide RNA to the target DUX4 RNA comprises a 6 nucleotide internal symmetric loop formed 1 nucleotide upstream (5′) from the target A, a 1 nucleotide mismatch formed 4 nucleotides downstream (3′) from the target A, and a 6 nucleotide internal symmetric loop formed 32 nucleotides downstream (3′) from the target A. In some cases, an engineered guide RNA of the present disclosure to a target DUX4 RNA has a sequence of SEQ ID NO: 1463 and, the guide-target RNA scaffold formed upon hybridization of said engineered guide RNA to the target DUX4 RNA comprises a 6 nucleotide internal symmetric loop formed 1 nucleotide upstream (5′) from the target A, a 1 nucleotide mismatch formed 4 nucleotides downstream (3′) from the target A, and a 6 nucleotide internal symmetric loop formed 32 nucleotides downstream (3′) from the target A.

In some cases, the structural feature formed upon hybridization of an engineered guide RNA of the present disclosure to a target DUX4 RNA comprises a 2 nucleotide symmetric bulge formed 7 nucleotides upstream (5′) from the target A, a 2 nucleotide symmetric bulge formed 6 nucleotides downstream (3′) from the target A, a 2 nucleotide symmetric bulge formed 20 nucleotides downstream (3′) from the target A, a 2 nucleotide symmetric bulge formed 34 nucleotides downstream (3′) from the target A, a 2 nucleotide symmetric bulge formed 48 nucleotides downstream (3′) from the target A, a 2 nucleotide symmetric bulge formed 62 nucleotides downstream (3′) from the target A, and a 2 nucleotide symmetric bulge formed 76 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1538 and, the guide-target RNA scaffold formed upon hybridization of said engineered guide RNA to the target DUX4 RNA comprises a 2 nucleotide symmetric bulge formed 7 nucleotides upstream (5′) from the target A, a 2 nucleotide symmetric bulge formed 6 nucleotides downstream (3′) from the target A, a 2 nucleotide symmetric bulge formed 20 nucleotides downstream (3′) from the target A, a 2 nucleotide symmetric bulge formed 34 nucleotides downstream (3′) from the target A, a 2 nucleotide symmetric bulge formed 48 nucleotides downstream (3′) from the target A, a 2 nucleotide symmetric bulge formed 62 nucleotides downstream (3′) from the target A, and a 2 nucleotide symmetric bulge formed 76 nucleotides downstream(3′) from the target A. In some cases, an engineered guide RNA of the present disclosure to a target DUX4 RNA has a sequence of SEQ ID NO: 1538 and, the guide-target RNA scaffold formed upon hybridization of said engineered guide RNA to the target DUX4 RNA comprises a 2 nucleotide symmetric bulge formed 7 nucleotides upstream (5′) from the target A, a 2 nucleotide symmetric bulge formed 6 nucleotides downstream (3′) from the target A, a 2 nucleotide symmetric bulge formed 20 nucleotides downstream (3′) from the target A, a 2 nucleotide symmetric bulge formed 34 nucleotides downstream (3′) from the target A, a 2 nucleotide symmetric bulge formed 48 nucleotides downstream (3′) from the target A, a 2 nucleotide symmetric bulge formed 62 nucleotides downstream (3′) from the target A, and a 2 nucleotide symmetric bulge formed 76 nucleotides downstream (3′) from the target A.

In some cases, the structural feature formed upon hybridization of an engineered guide RNA of the present disclosure to a target DUX4 RNA comprises a 3 nucleotide symmetric bulge formed 6 nucleotides upstream (5′) from the target A, a 3 nucleotide symmetric bulge formed 7 nucleotides downstream (3′) from the target A, a 3 nucleotide symmetric bulge formed 22 nucleotides downstream (3′) from the target A, a 3 nucleotide symmetric bulge formed 37 nucleotides downstream (3′) from the target A, a 3 nucleotide symmetric bulge formed 52 nucleotides downstream (3′) from the target A, and a 3 nucleotide symmetric bulge formed 67 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1539 and, the guide-target RNA scaffold formed upon hybridization of said engineered guide RNA to the target DUX4 RNA comprises a 3 nucleotide symmetric bulge formed 6 nucleotides upstream (5′) from the target A, a 3 nucleotide symmetric bulge formed 7 nucleotides downstream (3′) from the target A, a 3 nucleotide symmetric bulge formed 22 nucleotides downstream (3′) from the target A, a 3 nucleotide symmetric bulge formed 37 nucleotides downstream (3′) from the target A, a 3 nucleotide symmetric bulge formed 52 nucleotides downstream (3′) from the target A, and a 3 nucleotide symmetric bulge formed 67 nucleotides downstream (3′) from the target A. In some cases, an engineered guide RNA of the present disclosure to a target DUX4 RNA has a sequence of SEQ ID NO: 1539 and, the guide-target RNA scaffold formed upon hybridization of said engineered guide RNA to the target DUX4 RNA comprises a 3 nucleotide symmetric bulge formed 6 nucleotides upstream (5′) from the target A, a 3 nucleotide symmetric bulge formed 7 nucleotides downstream (3′) from the target A, a 3 nucleotide symmetric bulge formed 22 nucleotides downstream (3′) from the target A, a 3 nucleotide symmetric bulge formed 37 nucleotides downstream (3′) from the target A, a 3 nucleotide symmetric bulge formed 52 nucleotides downstream (3′) from the target A, and a 3 nucleotide symmetric bulge formed 67 nucleotides downstream(3′) from the target A.

In some cases, the structural feature formed upon hybridization of an engineered guide RNA of the present disclosure to a target DUX4 RNA comprises a 2 nucleotide symmetric bulge formed 5 nucleotides upstream (5′) from the target A, a 2 nucleotide symmetric bulge formed 10 nucleotides downstream (3′) from the target A, a 2 nucleotide symmetric bulge formed 26 nucleotides downstream (3′) from the target A, a 2 nucleotide symmetric bulge formed 42 nucleotides downstream (3′) from the target A, a 2 nucleotide symmetric bulge formed 58 nucleotides downstream (3′) from the target A, and a 2 nucleotide symmetric bulge formed 74 nucleotides downstream(3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1545 and, the guide-target RNA scaffold formed upon hybridization of said engineered guide RNA to the target DUX4 RNA comprises a 2 nucleotide symmetric bulge formed 5 nucleotides upstream (5′) from the target A, a 2 nucleotide symmetric bulge formed 10 nucleotides downstream (3′) from the target A, a 2 nucleotide symmetric bulge formed 26 nucleotides downstream (3′) from the target A, a 2 nucleotide symmetric bulge formed 42 nucleotides downstream (3′) from the target A, a 2 nucleotide symmetric bulge formed 58 nucleotides downstream (3′) from the target A, and a 2 nucleotide symmetric bulge formed 74 nucleotides downstream(3′) from the target A. In some cases, an engineered guide RNA of the present disclosure to a target DUX4 RNA has a sequence of SEQ ID NO: 1545 and, the guide-target RNA scaffold formed upon hybridization of said engineered guide RNA to the target DUX4 RNA comprises a 2 nucleotide symmetric bulge formed 5 nucleotides upstream (5′) from the target A, a 2 nucleotide symmetric bulge formed 10 nucleotides downstream (3′) from the target A, a 2 nucleotide symmetric bulge formed 26 nucleotides downstream (3′) from the target A, a 2 nucleotide symmetric bulge formed 42 nucleotides downstream (3′) from the target A, a 2 nucleotide symmetric bulge formed 58 nucleotides downstream (3′) from the target A, and a 2 nucleotide symmetric bulge formed 74 nucleotides downstream(3′) from the target A.

In some cases, the structural feature formed upon hybridization of an engineered guide RNA of the present disclosure to a target DUX4 RNA comprises a 2 nucleotide symmetric bulge formed 3 nucleotides upstream (5′) from the target A, a 2 nucleotide symmetric bulge formed 14 nucleotides downstream (3′) from the target A, a 2 nucleotide symmetric bulge formed 32 nucleotides downstream (3′) from the target A, a 2 nucleotide symmetric bulge formed 50 nucleotides downstream (3′) from the target A, and a 2 nucleotide symmetric bulge formed 68 nucleotides downstream(3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1552 and, the guide-target RNA scaffold formed upon hybridization of said engineered guide RNA to the target DUX4 RNA comprises a 2 nucleotide symmetric bulge formed 3 nucleotides upstream (5′) from the target A, a 2 nucleotide symmetric bulge formed 14 nucleotides downstream (3′) from the target A, a 2 nucleotide symmetric bulge formed 32 nucleotides downstream (3′) from the target A, a 2 nucleotide symmetric bulge formed 50 nucleotides downstream (3′) from the target A, and a 2 nucleotide symmetric bulge formed 68 nucleotides downstream(3′) from the target A. In some cases, an engineered guide RNA of the present disclosure to a target DUX4 RNA has a sequence of SEQ ID NO: 1552 and, the guide-target RNA scaffold formed upon hybridization of said engineered guide RNA to the target DUX4 RNA comprises a 2 nucleotide symmetric bulge formed 3 nucleotides upstream (5′) from the target A, a 2 nucleotide symmetric bulge formed 14 nucleotides downstream (3′) from the target A, a 2 nucleotide symmetric bulge formed 32 nucleotides downstream (3′) from the target A, a 2 nucleotide symmetric bulge formed 50 nucleotides downstream (3′) from the target A, and a 2 nucleotide symmetric bulge formed 68 nucleotides downstream(3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 45 nucleotides downstream (3′) from the target A, a 2 nucleotide symmetric bulge formed 53 nucleotides downstream (3′) from the target A, a 2 nucleotide symmetric bulge formed 61 nucleotides downstream (3′) from the target A, a 2 nucleotide symmetric bulge formed 69 nucleotides downstream (3′) from the target A, and a 2 nucleotide symmetric bulge formed 77 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1566 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 45 nucleotides downstream (3′) from the target A, a 2 nucleotide symmetric bulge formed 53 nucleotides downstream (3′) from the target A, a 2 nucleotide symmetric bulge formed 61 nucleotides downstream (3′) from the target A, a 2 nucleotide symmetric bulge formed 69 nucleotides downstream (3′) from the target A, and a 2 nucleotide symmetric bulge formed 77 nucleotides downstream (3′) from the target A. In some cases, an engineered guide RNA of the present disclosure to a target DUX4 RNA has a sequence of SEQ ID NO: 1566 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 45 nucleotides downstream (3′) from the target A, a 2 nucleotide symmetric bulge formed 53 nucleotides downstream (3′) from the target A, a 2 nucleotide symmetric bulge formed 61 nucleotides downstream (3′) from the target A, a 2 nucleotide symmetric bulge formed 69 nucleotides downstream (3′) from the target A, and a 2 nucleotide symmetric bulge formed 77 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 45 nucleotides downstream (3′) from the target A, a 3 nucleotide symmetric bulge formed 54 nucleotides downstream (3′) from the target A, a 3 nucleotide symmetric bulge formed 63 nucleotides downstream (3′) from the target A, and a 3 nucleotide symmetric bulge formed 72 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1567 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 45 nucleotides downstream (3′) from the target A, a 3 nucleotide symmetric bulge formed 54 nucleotides downstream (3′) from the target A, a 3 nucleotide symmetric bulge formed 63 nucleotides downstream (3′) from the target A, and a 3 nucleotide symmetric bulge formed 72 nucleotides downstream (3′) from the target A. In some cases, an engineered guide RNA of the present disclosure to a target DUX4 RNA has a sequence of SEQ ID NO: 1567 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 45 nucleotides downstream (3′) from the target A, a 3 nucleotide symmetric bulge formed 54 nucleotides downstream (3′) from the target A, a 3 nucleotide symmetric bulge formed 63 nucleotides downstream (3′) from the target A, and a 3 nucleotide symmetric bulge formed 72 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 45 nucleotides downstream (3′) from the target A, a 4 nucleotide symmetric bulge formed 55 nucleotides downstream (3′) from the target A, a 4 nucleotide symmetric bulge formed 65 nucleotides downstream (3′) from the target A, and a 4 nucleotide symmetric bulge formed 75 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1568 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 45 nucleotides downstream (3′) from the target A, a 4 nucleotide symmetric bulge formed 55 nucleotides downstream (3′) from the target A, a 4 nucleotide symmetric bulge formed 65 nucleotides downstream (3′) from the target A, and a 4 nucleotide symmetric bulge formed 75 nucleotides downstream (3′) from the target A. In some cases, an engineered guide RNA of the present disclosure to a target DUX4 RNA has a sequence of SEQ ID NO: 1568 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 45 nucleotides downstream (3′) from the target A, a 4 nucleotide symmetric bulge formed 55 nucleotides downstream (3′) from the target A, a 4 nucleotide symmetric bulge formed 65 nucleotides downstream (3′) from the target A, and a 4 nucleotide symmetric bulge formed 75 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 45 nucleotides downstream (3′) from the target A, a 5 nucleotide internal symmetric loop formed 56 nucleotides downstream (3′) from the target A, and a 5 nucleotide internal symmetric loop formed 67 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1569 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 45 nucleotides downstream (3′) from the target A, a 5 nucleotide internal symmetric loop formed 56 nucleotides downstream (3′) from the target A, and a 5 nucleotide internal symmetric loop formed 67 nucleotides downstream (3′) from the target A. In some cases, an engineered guide RNA of the present disclosure to a target DUX4 RNA has a sequence of SEQ ID NO: 1569 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 45 nucleotides downstream (3′) from the target A, a 5 nucleotide internal symmetric loop formed 56 nucleotides downstream (3′) from the target A, and a 5 nucleotide internal symmetric loop formed 67 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 47 nucleotides downstream (3′) from the target A, a 2 nucleotide symmetric bulge formed 57 nucleotides downstream (3′) from the target A, a 2 nucleotide symmetric bulge formed 67 nucleotides downstream (3′) from the target A, and a 2 nucleotide symmetric bulge formed 77 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1570 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 47 nucleotides downstream (3′) from the target A, a 2 nucleotide symmetric bulge formed 57 nucleotides downstream (3′) from the target A, a 2 nucleotide symmetric bulge formed 67 nucleotides downstream (3′) from the target A, and a 2 nucleotide symmetric bulge formed 77 nucleotides downstream (3′) from the target A. In some cases, an engineered guide RNA of the present disclosure to a target DUX4 RNA has a sequence of SEQ ID NO: 1570 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 47 nucleotides downstream (3′) from the target A, a 2 nucleotide symmetric bulge formed 57 nucleotides downstream (3′) from the target A, a 2 nucleotide symmetric bulge formed 67 nucleotides downstream (3′) from the target A, and a 2 nucleotide symmetric bulge formed 77 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 47 nucleotides downstream (3′) from the target A, a 3 nucleotide symmetric bulge formed 58 nucleotides downstream (3′) from the target A, and a 3 nucleotide symmetric bulge formed 69 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1571 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 47 nucleotides downstream (3′) from the target A, a 3 nucleotide symmetric bulge formed 58 nucleotides downstream (3′) from the target A, and a 3 nucleotide symmetric bulge formed 69 nucleotides downstream (3′) from the target A. In some cases, an engineered guide RNA of the present disclosure to a target DUX4 RNA has a sequence of SEQ ID NO: 1571 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 47 nucleotides downstream (3′) from the target A, a 3 nucleotide symmetric bulge formed 58 nucleotides downstream (3′) from the target A, and a 3 nucleotide symmetric bulge formed 69 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 47 nucleotides downstream (3′) from the target A, a 4 nucleotide symmetric bulge formed 59 nucleotides downstream (3′) from the target A, and a 4 nucleotide symmetric bulge formed 71 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1572 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 47 nucleotides downstream (3′) from the target A, a 4 nucleotide symmetric bulge formed 59 nucleotides downstream (3′) from the target A, and a 4 nucleotide symmetric bulge formed 71 nucleotides downstream (3′) from the target A. In some cases, an engineered guide RNA of the present disclosure to a target DUX4 RNA has a sequence of SEQ ID NO: 1572 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 47 nucleotides downstream (3′) from the target A, a 4 nucleotide symmetric bulge formed 59 nucleotides downstream (3′) from the target A, and a 4 nucleotide symmetric bulge formed 71 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 47 nucleotides downstream (3′) from the target A, a 5 nucleotide internal symmetric loop formed 60 nucleotides downstream (3′) from the target A, and a 5 nucleotide internal symmetric loop formed 73 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1573 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 47 nucleotides downstream (3′) from the target A, a 5 nucleotide internal symmetric loop formed 60 nucleotides downstream (3′) from the target A, and a 5 nucleotide internal symmetric loop formed 73 nucleotides downstream (3′) from the target A. In some cases, an engineered guide RNA of the present disclosure to a target DUX4 RNA has a sequence of SEQ ID NO: 1573 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 47 nucleotides downstream (3′) from the target A, a 5 nucleotide internal symmetric loop formed 60 nucleotides downstream (3′) from the target A, and a 5 nucleotide internal symmetric loop formed 73 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 49 nucleotides downstream (3′) from the target A, a 2 nucleotide symmetric bulge formed 61 nucleotides downstream (3′) from the target A, and a 2 nucleotide symmetric bulge formed 73 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1574 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 49 nucleotides downstream (3′) from the target A, a 2 nucleotide symmetric bulge formed 61 nucleotides downstream (3′) from the target A, and a 2 nucleotide symmetric bulge formed 73 nucleotides downstream (3′) from the target A. In some cases, an engineered guide RNA of the present disclosure to a target DUX4 RNA has a sequence of SEQ ID NO: 1574 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 49 nucleotides downstream (3′) from the target A, a 2 nucleotide symmetric bulge formed 61 nucleotides downstream (3′) from the target A, and a 2 nucleotide symmetric bulge formed 73 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 49 nucleotides downstream (3′) from the target A, a 3 nucleotide symmetric bulge formed 62 nucleotides downstream (3′) from the target A, and a 3 nucleotide symmetric bulge formed 75 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1575 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 49 nucleotides downstream (3′) from the target A, a 3 nucleotide symmetric bulge formed 62 nucleotides downstream (3′) from the target A, and a 3 nucleotide symmetric bulge formed 75 nucleotides downstream (3′) from the target A. In some cases, an engineered guide RNA of the present disclosure to a target DUX4 RNA has a sequence of SEQ ID NO: 1575 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 49 nucleotides downstream (3′) from the target A, a 3 nucleotide symmetric bulge formed 62 nucleotides downstream (3′) from the target A, and a 3 nucleotide symmetric bulge formed 75 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 49 nucleotides downstream (3′) from the target A, and a 4 nucleotide symmetric bulge formed 63 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1576 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 49 nucleotides downstream (3′) from the target A, and a 4 nucleotide symmetric bulge formed 63 nucleotides downstream (3′) from the target A. In some cases, an engineered guide RNA of the present disclosure to a target DUX4 RNA has a sequence of SEQ ID NO: 1576 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 49 nucleotides downstream (3′) from the target A, and a 4 nucleotide symmetric bulge formed 63 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 49 nucleotides downstream (3′) from the target A, and a 5 nucleotide internal symmetric loop formed 64 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1577 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 49 nucleotides downstream (3′) from the target A, and a 5 nucleotide internal symmetric loop formed 64 nucleotides downstream (3′) from the target A. In some cases, an engineered guide RNA of the present disclosure to a target DUX4 RNA has a sequence of SEQ ID NO: 1577 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 49 nucleotides downstream (3′) from the target A, and a 5 nucleotide internal symmetric loop formed 64 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 51 nucleotides downstream (3′) from the target A, and a 2 nucleotide symmetric bulge formed 65 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1578 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 51 nucleotides downstream (3′) from the target A, and a 2 nucleotide symmetric bulge formed 65 nucleotides downstream (3′) from the target A. In some cases, an engineered guide RNA of the present disclosure to a target DUX4 RNA has a sequence of SEQ ID NO: 1578 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 51 nucleotides downstream (3′) from the target A, and a 2 nucleotide symmetric bulge formed 65 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 51 nucleotides downstream (3′) from the target A, and a 3 nucleotide symmetric bulge formed 66 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1579 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 51 nucleotides downstream (3′) from the target A, and a 3 nucleotide symmetric bulge formed 66 nucleotides downstream (3′) from the target A. In some cases, an engineered guide RNA of the present disclosure to a target DUX4 RNA has a sequence of SEQ ID NO: 1579 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 51 nucleotides downstream (3′) from the target A, and a 3 nucleotide symmetric bulge formed 66 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 51 nucleotides downstream (3′) from the target A, and a 4 nucleotide symmetric bulge formed 67 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1580 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 51 nucleotides downstream (3′) from the target A, and a 4 nucleotide symmetric bulge formed 67 nucleotides downstream (3′) from the target A. In some cases, an engineered guide RNA of the present disclosure to a target DUX4 RNA has a sequence of SEQ ID NO: 1580 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 51 nucleotides downstream (3′) from the target A, and a 4 nucleotide symmetric bulge formed 67 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 51 nucleotides downstream (3′) from the target A, and a 5 nucleotide internal symmetric loop formed 68 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1581 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 51 nucleotides downstream (3′) from the target A, and a 5 nucleotide internal symmetric loop formed 68 nucleotides downstream (3′) from the target A. In some cases, an engineered guide RNA of the present disclosure to a target DUX4 RNA has a sequence of SEQ ID NO: 1581 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 51 nucleotides downstream (3′) from the target A, and a 5 nucleotide internal symmetric loop formed 68 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 53 nucleotides downstream (3′) from the target A, and a 2 nucleotide symmetric bulge formed 69 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1582 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 53 nucleotides downstream (3′) from the target A, and a 2 nucleotide symmetric bulge formed 69 nucleotides downstream (3′) from the target A. In some cases, an engineered guide RNA of the present disclosure to a target DUX4 RNA has a sequence of SEQ ID NO: 1582 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 53 nucleotides downstream (3′) from the target A, and a 2 nucleotide symmetric bulge formed 69 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 53 nucleotides downstream (3′) from the target A, and a 3 nucleotide symmetric bulge formed 70 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1583 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 53 nucleotides downstream (3′) from the target A, and a 3 nucleotide symmetric bulge formed 70 nucleotides downstream (3′) from the target A. In some cases, an engineered guide RNA of the present disclosure to a target DUX4 RNA has a sequence of SEQ ID NO: 1583 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 53 nucleotides downstream (3′) from the target A, and a 3 nucleotide symmetric bulge formed 70 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 53 nucleotides downstream (3′) from the target A, and a 4 nucleotide symmetric bulge formed 71 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1584 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 53 nucleotides downstream (3′) from the target A, and a 4 nucleotide symmetric bulge formed 71 nucleotides downstream (3′) from the target A. In some cases, an engineered guide RNA of the present disclosure to a target DUX4 RNA has a sequence of SEQ ID NO: 1584 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 53 nucleotides downstream (3′) from the target A, and a 4 nucleotide symmetric bulge formed 71 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 53 nucleotides downstream (3′) from the target A, and a 5 nucleotide internal symmetric loop formed 72 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1585 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 53 nucleotides downstream (3′) from the target A, and a 5 nucleotide internal symmetric loop formed 72 nucleotides downstream (3′) from the target A. In some cases, an engineered guide RNA of the present disclosure to a target DUX4 RNA has a sequence of SEQ ID NO: 1585 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 5 nucleotide internal symmetric loop formed 53 nucleotides downstream (3′) from the target A, and a 5 nucleotide internal symmetric loop formed 72 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 55 nucleotides downstream (3′) from the target A, and a 2 nucleotide symmetric bulge formed 73 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1586 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 55 nucleotides downstream (3′) from the target A, and a 2 nucleotide symmetric bulge formed 73 nucleotides downstream (3′) from the target A. In some cases, an engineered guide RNA of the present disclosure to a target DUX4 RNA has a sequence of SEQ ID NO: 1586 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 2 nucleotide symmetric bulge formed 55 nucleotides downstream (3′) from the target A, and a 2 nucleotide symmetric bulge formed 73 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 55 nucleotides downstream (3′) from the target A, and a 3 nucleotide symmetric bulge formed 74 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1587 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 55 nucleotides downstream (3′) from the target A, and a 3 nucleotide symmetric bulge formed 74 nucleotides downstream (3′) from the target A. In some cases, an engineered guide RNA of the present disclosure to a target DUX4 RNA has a sequence of SEQ ID NO: 1587 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 3 nucleotide symmetric bulge formed 55 nucleotides downstream (3′) from the target A, and a 3 nucleotide symmetric bulge formed 74 nucleotides downstream (3′) from the target A.

In some cases, the structural feature formed upon hybridization of an engineered guide RNA of the present disclosure to a target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 55 nucleotides downstream (3′) from the target A, and a 4 nucleotide symmetric bulge formed 75 nucleotides downstream (3′) from the target A. In some cases, an engineered guide RNA of the present disclosure to a target DUX4 RNA has at least 80%, 85%, 90%, 92%, 95%, 97%, or 99% sequence identity to a guide RNA comprising SEQ ID NO: 1588 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 55 nucleotides downstream (3′) from the target A, and a 4 nucleotide symmetric bulge formed 75 nucleotides downstream (3′) from the target A. In some cases, an engineered guide RNA of the present disclosure to a target DUX4 RNA has a sequence of SEQ ID NO: 1588 and, the guide-target RNA scaffold formed upon hybridization of said engineered guide RNA to the target DUX4 RNA comprises a 6 nucleotide internal symmetric loop formed 6 nucleotides upstream (5′) from the target A, a 1 nucleotide mismatch formed 3 nucleotides downstream (3′) from the target A, a 6 nucleotide internal symmetric loop formed 33 nucleotides downstream (3′) from the target A, a 4 nucleotide symmetric bulge formed 55 nucleotides downstream (3′) from the target A, and a 4 nucleotide symmetric bulge formed 75 nucleotides downstream (3′) from the target A.

In some cases, the structural feature formed upon hybridization of an engineered guide RNA of the present disclosure to a target RNA comprises a 6 nucleotide internal symmetric loop formed downstream (3′) from the target A.

In some cases, the structural feature formed upon hybridization of an engineered guide RNA of the present disclosure to a target RNA comprises a 6 nucleotide internal symmetric loop formed upstream (5′) from the target A and a 6 nucleotide internal symmetric loop formed downstream (3′) from the target A.

In some cases, the structural feature formed upon hybridization of an engineered guide RNA of the present disclosure to a target RNA comprises a 1 nucleotide mismatch formed downstream (3′) from the target A, and a 6 nucleotide internal symmetric loop formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed at the target A, and a 6 nucleotide internal symmetric loop formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, and one 6 nucleotide internal symmetric loop formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, and two 6 nucleotide internal symmetric loop(s) formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, and three 6 nucleotide internal symmetric loop(s) formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, and four 6 nucleotide internal symmetric loop(s) formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and two 2 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and three 2 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and four 2 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and five 2 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and two 3 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and three 3 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and four 3 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and two 4 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and three 4 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and four 4 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and two 5 nucleotide symmetric bulges formed downstream (3′) from the target A.

In some cases, the structural feature formed upon hybridization of an engineered guide RNA of the present disclosure to a target RNA comprises a 6 nucleotide internal symmetric loop formed upstream (5′) from the target A, a 1 nucleotide mismatch formed downstream (3′) from the target A, a 6 nucleotide internal symmetric loop formed downstream (3′) from the target A, and three 5 nucleotide symmetric bulges formed downstream (3′) from the target A.

In some cases, the structural feature formed upon hybridization of an engineered guide RNA of the present disclosure to a target RNA comprises a 2 nucleotide symmetric bulge upstream (5′) from the target A and four 2 nucleotide symmetric bulges downstream (3′) from the target A.

In some cases, the structural feature formed upon hybridization of an engineered guide RNA of the present disclosure to a target RNA comprises a 2 nucleotide symmetric bulge upstream (5′) from the target A and five 2 nucleotide symmetric bulges downstream (3′) from the target A.

In some cases, the structural feature formed upon hybridization of an engineered guide RNA of the present disclosure to a target RNA comprises a 2 nucleotide symmetric bulge upstream (5′) from the target A and six 2 nucleotide symmetric bulges downstream (3′) from the target A.

### D. Additional Engineered Guide RNA Components

The present disclosure provides for engineered guide RNAs with additional structural features and components. For example, an engineered guide RNA described herein can be circular. In another example, an engineered guide RNA described herein can comprise a U7, an SmOPT sequence, or a combination of both.

In some cases, an engineered guide RNA can be circularized. In some cases, an engineered guide RNA provided herein can be circularized or in a circular configuration. In some aspects, an at least partially circular guide RNA lacks a 5′ hydroxyl or a 3′ hydroxyl. In some embodiments, a circular engineered guide RNA can comprise a guide RNA from any one of SEQ ID NOs: 2-1589.

In some examples, an engineered guide RNA can comprise a backbone comprising a plurality of sugar and phosphate moieties covalently linked together. In some examples, a backbone of an engineered guide RNA can comprise a phosphodiester bond linkage between a first hydroxyl group in a phosphate group on a 5′ carbon of a deoxyribose in DNA or ribose in RNA and a second hydroxyl group on a 3′ carbon of a deoxyribose in DNA or ribose in RNA.

In some embodiments, a backbone of an engineered guide RNA can lack a 5′ reducing hydroxyl, a 3′ reducing hydroxyl, or both, capable of being exposed to a solvent. In some embodiments, a backbone of an engineered guide can lack a 5′ reducing hydroxyl, a 3′ reducing hydroxyl, or both, capable of being exposed to nucleases. In some embodiments, a backbone of an engineered guide can lack a 5′ reducing hydroxyl, a 3′ reducing hydroxyl, or both, capable of being exposed to hydrolytic enzymes. In some instances, a backbone of an engineered guide can be represented as a polynucleotide sequence in a circular 2-dimensional format with one nucleotide after the other. In some instances, a backbone of an engineered guide can be represented as a polynucleotide sequence in a looped 2-dimensional format with one nucleotide after the other. In some cases, a 5′ hydroxyl, a 3′ hydroxyl, or both, can be joined through a phosphorus-oxygen bond. In some cases, a 5′ hydroxyl, a 3′ hydroxyl, or both, can be modified into a phosphoester with a phosphorus-containing moiety.

As described herein, an engineered guide can comprise a circular structure. An engineered polynucleotide can be circularized from a precursor engineered polynucleotide. Such a precursor engineered polynucleotide can be a precursor engineered linear polynucleotide. In some cases, a precursor engineered linear polynucleotide can be a precursor for a circular engineered guide RNA. For example, a precursor engineered linear polynucleotide can be a linear mRNA transcribed from a plasmid, which can be configured to circularize within a cell using the techniques described herein. A precursor engineered linear polynucleotide can be constructed with domains such as a ribozyme domain and a ligation domain that allow for circularization when inserted into a cell. A ribozyme domain can include a domain that is capable of cleaving the linear precursor RNA at specific sites (e.g., adjacent to the ligation domain). A precursor engineered linear polynucleotide can comprise, from 5′ to 3′: a 5′ ribozyme domain, a 5′ ligation domain, a circularized region, a 3′ ligation domain, and a 3′ ribozyme domain. In some cases, a circularized region can comprise a guide RNA described herein. In some cases, the precursor polynucleotide can be specifically processed at both sites by the 5′ and the 3′ ribozymes, respectively, to free exposed ends on the 5′ and 3′ ligation domains. The free exposed ends can be ligation competent, such that the ends can be ligated to form a mature circularized structure. For instance, the free ends can include a 5′-OH and a 2′, 3′-cyclic phosphate that are ligated via RNA ligation in the cell. The linear polynucleotide with the ligation and ribozyme domains can be transfected into a cell where it can circularize via endogenous cellular enzymes. In some cases, a polynucleotide can encode an engineered guide RNA comprising the ribozyme and ligation domains described herein, which can circularize within a cell. Circular guide RNAs are described in PCT/US2021/034301, which is incorporated by reference in its entirety.

An engineered polynucleotide as described herein (e.g., a circularized guide RNA) can include spacer domains. As described herein, a spacer domain can refer to a domain that provides space between other domains. A spacer domain can be used to between a region to be circularized and flanking ligation sequences to increase the overall size of the mature circularized guide RNA. Where the region to be circularized includes a targeting domain as described herein that is configured to associate to a target sequence, the addition of spacers can provide improvements (e.g. increased specificity, enhanced editing efficiency, etc.) for the engineered polynucleotide to the target polynucleotide, relative to a comparable engineered polynucleotide that lacks a spacer domain. In some instances, the spacer domain is configured to not hybridize with the target RNA. In some embodiments, a precursor engineered polynucleotide or a circular engineered guide, can comprise, in order of 5′ to 3′: a first ribozyme domain; a first ligation domain; a first spacer domain; a targeting domain that can be at least partially complementary to a target RNA, a second spacer domain, a second ligation domain, and a second ribozyme domain. In some cases, the first spacer domain, the second spacer domain, or both are configured to not bind to the target RNA when the targeting domain binds to the target RNA.

The compositions and methods of the present disclosure provide engineered polynucleotides encoding for guide RNAs that are operably linked to a portion of a small nuclear ribonucleic acid (snRNA) sequence. The engineered polynucleotide can include at least a portion of a small nuclear ribonucleic acid (snRNA) sequence. The U7 and U1 small nuclear RNAs, whose natural role is in spliceosomal processing of pre-mRNA, have for decades been re-engineered to alter splicing at desired disease targets. Replacing the first 18 nt of the U7 snRNA (which naturally hybridizes to the spacer element of histone pre-mRNA) with a short targeting (or antisense) sequence of a disease gene, redirects the splicing machinery to alter splicing around that target site. Furthermore, converting the wild type U7 Sm-domain binding site to an optimized consensus Sm-binding sequence (SmOPT) can increase the expression level, activity, and subcellular localization of the artificial antisense-engineered U7 snRNA. Many subsequent groups have adapted this modified U7 SmOPT snRNA chassis with antisense sequences of other genes to recruit spliceosomal elements and modify RNA splicing for additional disease targets.

An snRNA is a class of small RNA molecules found within the nucleus of eukaryotic cells. They are involved in a variety of important processes such as RNA splicing (removal of introns from pre-mRNA), regulation of transcription factors (7SK RNA) or RNA polymerase II (B2 RNA), and maintaining the telomeres. They are always associated with specific proteins, and the resulting RNA-protein complexes are referred to as small nuclear ribonucleoproteins (snRNP) or sometimes as snurps. There are many snRNAs, which are denominated U1, U2, U3, U4, U5, U6, U7, U8, U9, and U10.

The snRNA of the U7 type is normally involved in the maturation of histone mRNA. This snRNA has been identified in a great number of eukaryotic species (56 so far) and the U7 snRNA of each of these species should be regarded as equally convenient for this disclosure.

Wild-type U7 snRNA includes a stem-loop structure, the U7-specific Sm sequence, and a sequence antisense to the 3′ end of histone pre-mRNA.

In addition to the SmOPT domain, U7 comprises a sequence antisense to the 3′ end of histone pre-mRNA. When this sequence is replaced by a targeting sequence that is antisense to another target pre-mRNA, U7 is redirected to the new target pre-mRNA. Accordingly, the stable expression of modified U7 snRNAs containing the SmOPT domain and a targeting antisense sequence has resulted in specific alteration of mRNA splicing. While AAV-2/1 based vectors expressing an appropriately modified murine U7 gene along with its natural promoter and 3′ elements have enabled high efficiency gene transfer into the skeletal muscle and complete dystrophin rescue by covering and skipping mouse Dmd exon 23, the engineered polynucleotides as described herein (whether directly administered or administered via, for example, AAV vectors) can facilitate editing of target RNA by a deaminase.

The engineered polynucleotide can comprise at least in part an snRNA sequence. The snRNA sequence can be U1, U2, U3, U4, U5, U6, U7, U8, U9, or a U10 snRNA sequence.

In some instances, an engineered polynucleotide that comprises at least a portion of an snRNA sequence (e.g. an snRNA promoter, an snRNA hairpin, and the like) can have superior properties for treating or preventing a disease or condition, relative to a comparable polynucleotide lacking such features. For example, as described herein an engineered polynucleotide that comprises at least a portion of an snRNA sequence can facilitate exon skipping of an exon at a greater efficiency than a comparable polynucleotide lacking such features. Further, as described herein an engineered polynucleotide that comprises at least a portion of an snRNA sequence can facilitate an editing of a base of a nucleotide in a target RNA (e.g. a pre-mRNA or a mature RNA) at a greater efficiency than a comparable polynucleotide lacking such features. Promoters and snRNA components are described in PCT/US2021/028618, which is incorporated by reference in its entirety.

Disclosed herein are engineered RNAs comprising (a) an engineered guide RNA as described herein, and (b) a U7 snRNA hairpin sequence, a SmOPT sequence, or a combination thereof. In some embodiments, the U7 hairpin comprises a human U7 Hairpin sequence, or a mouse U7 hairpin sequence. In some cases, a human U7 hairpin sequence comprises TAGGCTTTCTGGCTTTTTACCGGAAAGCCCCT (SEQ ID NO: 1590 or RNA: UAGGCUUUCUGGCUUUUUACCGGAAAGCCCCU (SEQ ID NO: 1591). In some cases, a mouse U7 hairpin sequence comprises CAGGTTTTCTGACTTCGGTCGGAAAACCCCT (SEQ ID NO: 1592 or RNA: CAGGUUUUCUGACUUCGGUCGGAAAACCCCU SEQ ID NO: 1593). In some embodiments, the SmOPT sequence has a sequence of AATTTTTGGAG (SEQ ID NO: 1594 or RNA: AAUUUUUGGAG SEQ ID NO: 1595). In some embodiments, a guide RNA from any one of SEQ ID NOs: 2-1589 can comprise a guide RNA comprising a U7 hairpin sequence (e.g., a human or a mouse U7 hairpin sequence), an SmOPT sequence, or a combination thereof. In some cases, a combination of a U7 hairpin sequence and a SmOPT sequence can comprise a SmOPT U7 hairpin sequence, wherein the SmOPT sequence is linked to the U7 sequence. In some cases, a U7 hairpin sequence, an SmOPT sequence, or a combination thereof is downstream (e.g., 3′) of the engineered guide RNA disclosed herein.

Also disclosed herein are promoters for driving the expression of a guide RNA disclosed herein. In some cases, the promoters for driving expression can be 5′ to the guide RNA sequence disclosed herein. In some cases, a promoter can comprise a U1 promoter, a U7 promoter, a U6 promoter or any combination thereof. In some cases, a promoter can comprise a CMV promoter. In some cases, a U7 promoter, or a U6 promoter can be a mouse U7 promoter, or a mouse U6 promoter. In some cases, a U1 promoter, a U7 promoter, or a U6 promoter can be a human U1 promoter, a human U7 promoter, or a human U6 promoter. In some cases, a human U6 promoter can comprise a sequence with at least about: 70%, 75%, 80%, 85%, 90%, 95%, or 99% sequence identity to:
GAGGGCCTATTTCCCATGATTCCTTCATATTTGCATATACGATACAAGGCTGTTA GAGAGATAATTAGAATTAATTTGACTGTAAACACAAAGATATTAGTACAAAATA CGTGACGTAGAAAGTAATAATTTCTTGGGTAGTTTGCAGTTTTAAAATTATGTTTT AAAATGGACTATCATATGCTTACCGTAACTTGAAAGTATTTCGATTTCTTGGCTTT ATATATCTTGTGGAAAGGACGAAACACC (SEQ ID NO: 1596). In some cases, a mouse U6 promoter can comprise a sequence with at least about: 70%, 75%, 80%, 85%, 90%, 95%, or 99% sequence identity to:
GTACTGAGTCGCCCAGTCTCAGATAGATCCGACGCCGCCATCTCTAGGCCCGCGC CGGCCCCCTCGCACAGACTTGTGGGAGAAGCTCGGCTACTCCCCTGCCCCGGTTA ATTTGCATATAATATTTCCTAGTAACTATAGAGGCTTAATGTGCGATAAAAGACA GATAATCTGTTCTTTTTAATACTAGCTACATTTTACATGATAGGCTTGGATTTCTA TAAGAGATACAAATACTAAATTATTATTTTAAAAAACAGCACAAAAGGAAACTC ACCCTAACTGTAAAGTAATTGTGTGTTTTGAGACTATAAATATCCCTTGGAGAAA AGCCTTGTTTG (SEQ ID NO: 1597). In some cases, a human U7 promoter can comprise a sequence with at least about: 70%, 75%, 80%, 85%, 90%, 95%, or 99% sequence identity to: TTAACAACATAGGAGCTGTGATTGGCTGTTTTCAGCCAATCAGCACTGACTCATT TGCATAGCCTTTACAAGCGGTCACAAACTCAAGAAACGAGCGGTTTTAATAGTCT TTTAGAATATTGTTTATCGAACCGAATAAGGAACTGTGCTTTGTGATTCACATAT CAGTGGAGGGGTGTGGAAATGGCACCTTGATCTCACCCTCATCGAAAGTGGAGT TGATGTCCTTCCCTGGCTCGCTACAGACGCACTTCCGC (SEQ ID NO: 1598). In some cases, a mouse U7 promoter can comprise a sequence with at least about: 70%, 75%, 80%, 85%, 90%, 95%, or 99% sequence identity to:
TTAACAACATAGGAGCTGTGATTGGCTGTTTTCAGCCAATCAGCACTGACTCATT TGCATAGCCTTTACAAGCGGTCACAAACTCAAGAAACGAGCGGTTTTAATAGTCT TTTAGAATATTGTTTATCGAACCGAATAAGGAACTGTGCTTTGTGATTCACATAT CAGTGGAGGGGTGTGGAAATGGCACCTTGATCTCACCCTCATCGAAAGTGGAGT TGATGTCCTTCCCTGGCTCGCTACAGACGCACTTCCGC (SEQ ID NO: 1599). In some cases, a human U1 promoter can comprise a sequence with at least about: 70%, 75%, 80%, 85%, 90%, 95%, or 99% sequence identity to:
TAAGGACCAGCTTCTTTGGGAGAGAACAGACGCAGGGGCGGGAGGGAAAAAGG GAGAGGCAGACGTCACTTCCTCTTGGCGACTCTGGCAGCAGATTGGTCGGTTGAG TGGCAGAAAGGCAGACGGGGACTGGGCAAGGCACTGTCGGTGACATCACGGAC AGGGCGACTTCTATGTAGATGAGGCAGCGCAGAGGCTGCTGCTTCGCCACTTGCT GCTTCGCCACGAAGGGAGTTCCCGTGCCCTGGGAGCGGGTTCAGGACCGCTGAT CGGAAGTGAGAATCCCAGCTGTGTGTCAGGGCTGGAAAGGGCTCGGGAGTGCGC GGGGCAAGTGACCGTGTGTGTAAAGAGTGAGGCGTATGAGGCTGTGTCGGGGCA GAGCCCGAAGATCTC (SEQ ID NO: 1600). In some cases, a CMV promoter can comprise a sequence with at least about: 70%, 75%, 80%, 85%, 90%, 95%, or 99% sequence identity to:

(SEQ ID NO: 1601)
ATACGCGTTGACATTGATTATTGACTAGTTATTAATAGTAATCAATTACG
GGGTCATTAGTTCATAGCCCATATATGGAGTTCCGCGTTACATAACTTAC
GGTAAATGGCCCGCCTGGCTGACCGCCCAACGACCCCCGCCCATTGACGT
CAATAATGACGTATGTTCCCATAGTAACGCCAATAGGGACTTTCCATTGA
CGTCAATGGGTGGAGTATTTACGGTAAACTGCCCACTTGGCAGTACATCA
AGTGTATCATATGCCAAGTACGCCCCCTATTGACGTCAATGACGGTAAAT
GGCCCGCCTGGCATTATGCCCAGTACATGACCTTATGGGACTTTCCTACT
TGGCAGTACATCTACGTATTAGTCATCGCTATTACCATGGTGATGCGGTT
TTGGCAGTACATCAATGGGCGTGGATAGCGGTTTGACTCACGGGGATTTC
CAAGTCTCCACCCCATTGACGTCAATGGGAGTTTGTTTTGGCACCAAAAT
CAACGGGACTTTCCAAAATGTCGTAACAACTCCGCCCCATTGACGCAAAT
GGGCGGTAGGCGTGTACGGTGGGAGGTCTATATAAGCAGAGCTCGTTTAG
TGAACCGTCAGATCGCCTGGAGACGCCATCCACGCTGTTTTGACCTCCAT
AGAAGACACCGGGACCGATCCAGCCTCCGGACTCTAGAGGATCGAACC.

### Targets and Methods of Treatment

The present disclosure provides for compositions of engineered guide RNAs or engineered polynucleotides encoding guide RNAs and methods of use thereof, such as methods of treatment. In some embodiments, the engineered polynucleotides of the present disclosure encode guide RNAs targeting a coding sequence of RNA (e.g., a TIS) or a non-coding sequence of RNA (e.g., a polyA signal sequence). In some embodiments, the present disclosure provides compositions or more than one engineered polynucleotides of encoding more than one engineered guide RNAs targeting the TIS and the polyA sequence. The engineered guide RNAs disclosed herein facilitate ADAR-mediated RNA editing of adenosines in the TIS, the polyA sequence, or both. In some embodiments, engineered guide RNAs disclosed herein can be screened by in vitro and in vivo methods to determine their ability to facilitate ADAR mediated RNA editing of adenosines in a target RNA. In some cases, a screening method can comprise cell based reporter assay as described herein.

DUX4. The present disclosure provides for engineered guide RNAs that facilitate RNA editing of DUX4-FL to knockdown expression of DUX4-FL mRNA and DUX4-activated genes, and hence DUX4 activity. Facioscapulohumeral muscular dystrophy (FSHD) is a rare neuromuscular disease characterized by progressive skeletal muscle weakness and wasting with significant heterogeneity in phenotypic severity and age of onset. FSHD affects mostly the face (facio), shoulder girdle (scapula), and upper arm (humeral) regions of the body. As the disease progresses, muscles of the upper arms, the legs, and the postural muscles in the back loose mass and strength. Patients often first present with weakness of the face and periscapular muscles, eventually resulting in the inability to raise their arms above shoulder height, make facial expressions, or even close their eyes. In about 20% of the patients with FSHD, paraspinal muscle weakness is debilitating enough to result in patients becoming wheelchair-bound. FSHD is one of the most prevalent adult muscular dystrophies caused by an epigenetic derepression of the subtelomeric D4Z4 microsatellite array on chromosome 4q. This epigenetic derepression leads to hypomethylation in the distal-most D4Z4 unit and misexpression of the DUX4 gene in skeletal muscle. There are two subtypes of FSHD—FSHD1 and FSHD2. FSHD1 accounts for 95% of FSHD cases and is associated with the pathogenic contraction of D4Z4 microsatellite repeats, while FSHD2 accounts for 5% of the FSHD cases and is contraction-independent but associated with mutations in the chromatin regulator gene SMCHD1. The mutations for both FSHD1 and FSHD2 result in derepression of D4Z4 array and DUX4 mRNA misexpression. Said DUX4 mutations are autosomal dominant in ⅔ of FSHD1 patients and is prevalent in 1:8,000-12,000 (˜16,000-38,000 patients in the US). DUX4 (double homeobox 4) is a germline transcription factor and its misexpression in muscle activates the expression of a broad set of genes (DUX4-activated genes), many involved in stem and germ cell biology. Some known DUX4-activated genes include MBD3L2, TRIM43, PRAMEF12, ZSCAN, and LEUTX. Although physical therapy, pain management, and surgery can alleviate some of the disabilities associated with FSHD, these treatments are not curative, and none of them address the underlying cause of the disease pathology. While healthy subjects generate a non-toxic splice form of DUX4 mRNA that lacks the C-term transactivation domain of DUX4 (referred to as DUX4-S for short), affected subjects produce a toxic splice form of DUX4 mRNA (referred to as DUX4-FL for full length) leading to expression of a toxic form of the DUX4 protein in muscle. Although various pharmaceutical and cell-based intervention approaches are being explored to treat FSHD, these generally offer little to no therapeutic benefit based on results from clinical trials. To develop a more targeted form of treatment, approaches that reduce muscle-specific DUX4-FL expression and DUX4-mediated toxicity have become attractive goals of FSHD therapy. Indeed, genetic treatments that target the root cause of the disease (e.g., DUX4) are expected to lead to a more effective or far-reaching therapeutic effect. The exact amount of DUX4 inhibition required for effective therapy is currently unknown, but data from clinically affected and asymptomatic FSHD patients support the idea that any reduction in DUX4-FL mRNA expression will have a therapeutic benefit. In some embodiments, the present disclosure provides compositions of engineered guide RNAs that target DUX4 and facilitate ADAR-mediated RNA editing of DUX4, specifically, DUX4-FL to mediate DUX4-FL knockdown. In some embodiments, the engineered guide RNAs of the present disclosure target a coding sequence in DUX4-FL. For example, the coding sequence can be a translation initiation site (TIS) (AUG) of DUX4 and the engineered guide RNA can facilitate ADAR-mediated RNA editing of AUG to GUG. In some embodiments, the engineered guide RNAs of the present disclosure target a splice site in DUX4 pre-mRNA. In some embodiments, the engineered guide RNAs of the present disclosure target a non-coding sequence in DUX4. The non-coding sequence can be a polyA signal sequence (ATTAAA) in the pLAM region and the engineered guide RNA can facilitate ADAR-mediated RNA editing of one or more adenosines in the polyA signal sequence of DUX4. RNA editing of this polyA signal sequence reduces polyadenylation and genetic excision of the DUX4-FL polyA sequence results in DUX4-FL mRNA knockdown and DUX4-FL protein knockdown. In some embodiments, engineered guide RNAs of the present disclosure can be multiplexed to target more than one polyA signal sequences in DUX4. In some embodiments, engineered guide RNAs of the present disclosure can be multiplexed to target the TIS and one or more polyA signal sequences in DUX4.

In some embodiments, a target tissue for a guide RNA targeting DUX4 can comprise a muscle. In some cases, a muscle can comprise a muscle of the face, an arm muscle, a neck muscle, a shoulder muscle, a thigh muscle, a hip muscle, an abdominal muscle, a back muscle, a foot muscle, a hand muscle, or any combination thereof. In some cases, a muscle can comprise an orbicularis oculi, an orbicularis oris, a risorius, a zygomaticus major and minor, a biceps brachii, a triceps brichii, a trapezius, a rhomboids, a levator scapulae, a latissimus dorsi, a pectorals major, a pelvic girdle muscles, an abdominal muscles, a tibialis anterior, or any combination thereof. In some cases, a muscle of the face can comprise an occipitofrontalis muscle, a orbicularis oculi muscle, a temporalis muscle, a buccinator muscle, a masseter muscle, a mentalis muscle, a depressor labii inferioris muscle, a orbicularis oris muscle, a levator anguli oris muscle, a levator labii superioris muscle, a depressor anguli oris muscle, a levator labii superioris alaeque nasi muscle, zygomaticus major and minor muscle, a orbicularis oculi muscle, a corrugator supercilii muscle, or a risorius muscle. In some cases, a neck muscle can comprise an omohyoid muscle, a platysma muscle, a sternohyoid muscle, a sternocleidomastoid muscle, a levator scapulae muscle, a scalene muscle, a trapezius muscle, a semispinalis capitis muscle, a serratus posterior superior muscle, or any combination thereof. In some cases, shoulder muscle can comprise a deltoid muscle, a supraspinatus muscle, a rhomboids muscle, an infraspinatus muscle, a teres minor muscle, a teres major muscle, a pectoralis major muscle, a pectoralis minor, a serratus anterior muscle, or any combination thereof. In some cases, an arm muscle can comprise a triceps brachii muscle, a biceps brachii muscle, a brachialis muscle, a brachioradialis muscle, a carpal muscle, an extensor digitorum muscle, a extensor indicis muscle, an extensor digiti minimi muscle, a flexor digitorum superficialis muscle, a flexor digitorum profundus muscle, flexor pollicis longus muscle, extensor pollicis longus muscle, extensor pollicis brevis muscle, abductor pollicis longus muscle, a thenar muscles muscle, an adductor pollicis muscle, a hypothenar muscles muscle, a lumbricales muscle, a dorsal interossei muscle, a palmar interossei muscle, or any combination thereof. In some cases, a hip muscle can comprise a tensor fasciae muscle, a gluteus minimus muscle, a gluteus maximus muscle, a gluteus medius muscle, a piriformis muscle, a obturator internus muscle, or any combination thereof. In some cases, an abdominal muscle can comprise a pyramidalis muscle, a rectus abdominus muscle, an external oblique muscle, an internal oblique muscle, a transversus abdominis muscle, or any combination thereof. In some cases, a back muscle can comprise a trapezius muscle, a rhomboids muscle, a latissimus dorsi muscle, an erector spinae muscle, a multifidus muscle, a quadratus lumborum muscle, or any combination thereof. In some cases, a leg muscle can comprise a vastus lateralis muscle, a vastus medialis muscle, a vastus intermedius muscle, a rectus femoris muscle, a biceps femoris muscle, a semimembranosus muscle, a semitendinosus muscle, a gastrocnemius muscle, a soleus muscle, a plantaris muscle, or any combination thereof. In some cases, a foot muscle can comprise an abductor hallucis muscle, a tibialis anterior muscle, an extensor digitorum longus muscle, a flexor digitorum longus muscle, a fibularis longus muscle, a fibularis tertius muscle, a fibularis brevis muscle, or any combination thereof.

In some embodiments, a target cell for a guide RNA targeting DUX4 can comprise a somatic (e.g., a muscle cell) or a gamete cell. For example, a somatic cell can comprise a cell of an internal organ, the skin, a muscle, a bone, a blood cell, a connective tissue cell, or any combination thereof. In some cases, a somatic cell can comprise a muscle cell. In some cases, a muscle cell can comprise a skeletal muscle cell, a cardiac muscle cell, a smooth muscle cell, or a combination thereof. In some cases, a muscle cell can comprise a myocyte, a myofibril, a myoblast, a cardiomyocyte, or any combination thereof.

The engineered guide RNAs of the present disclosure facilitated ADAR-mediated RNA editing of DUX4, thereby, affecting reporter protein knockdown. In some embodiments, the engineered guide RNAs of the present disclosure facilitated ADAR-mediated RNA editing of from 1 to 100% of a target adenosine. The engineered guide RNAs of the present disclosure can facilitate from 40 to 90% editing of a target adenosine. In some embodiments, the engineered guide RNAs of the present disclosure can facilitate at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, 100%, from 5 to 20%, from 20 to 40%, from 40 to 60%, from 60 to 80%, from 80 to 100%, from 60 to 80%, from 70 to 90%, or up to 90% or more RNA editing of a target adenosine. Optionally, additionally, the engineered guide RNAs of the present disclosure can facilitate these levels of on-target RNA editing while maintaining less than 10% editing of an off-target adenosine. Optionally, additionally, the engineered guide RNAs of the present disclosure can facilitate these levels of on-target RNA editing while maintaining less than less than 30%, less than 25%, less than 20%, less than 15%, less than 10%, less than 9%, less than 8%, less than 7%, less than 6%, less than 5%, less than 4%, less than 3%, less than 2%, less than 1%, or 0% editing of an off-target adenosine.

In some embodiments, the DUX4 RNA comprises a pre-mRNA transcript of DUX4. In some embodiments, an engineered guide RNA of the present disclosure can facilitate editing of at least one edit in the polyA signal sequence the pre-mRNA transcript of DUX4. In some cases, at least 40%, at least 50%, at least 55%, at least 60%, at least 65%, at least 70%, at least 75%, at least 80%, at least 85%, at least 90%, at least 95%, or at least 99% of the pre-mRNA transcripts of DUX4 have at least one edit in the polyA signal sequence. In some cases, at least 80%, of the pre-mRNA transcripts of DUX4 have at least one edit in the polyA signal sequence. In some cases, 1% to 10%, 10% to 20%, 20% to 30%, 30% to 40%, 40% to 50%, 50% to 60%, 60% to 70%, 70% to 80%, 80% to 90%, 90% to 100%, 20% to 40%, 30% to 50%, 40% to 60%, 50% to 70%, 60% to 80%, 20% to 50%, or 30% to 60% of the pre-mRNA transcripts of DUX4 have at least one edit in the polyA signal sequence.

In some embodiments, a mutation in the polyA signal sequence (ATTAAA) in the pLAM region of DUX4-FL results in a DUX4 mRNA knockdown, a DUX4 protein knockdown, or both. As RNA, the polyA signal sequence corresponds to the sequence AUUAAA. In some cases, the polyA signal sequence (AUUAAA) can be mutated to AUUAAG; AUUAGA; AUUGAA; GUUAAA; or GUUGGG. In some cases, an engineered guide RNA disclosed herein can facilitate ADAR-mediated RNA editing of the unmodified polyA signal sequence (AUUAAA) to AUUAAG; AUUAGA; AUUGAA; GUUAAA; or GUUGGG. In some instances, ADAR-mediated RNA editing of the unmodified polyA signal sequence to AUUAAG; AUUAGA; AUUGAA; GUUAAA; or GUUGGG results in a DUX4 mRNA knockdown, a DUX4 protein knockdown, or both.

In some embodiments, an engineered guide disclosed herein can facilitate ADAR-mediated RNA editing of one or more adenosines in the non-coding polyA signal sequence (ATTAAA) in the pLAM region of DUX4. In some cases, a method of editing DUX4 RNA can comprise contacting the DUX4 RNA with a engineered guide disclosed herein and an RNA editing entity. In some cases, the method can comprise editing the non-coding polyA signal sequence. As RNA, the polyA signal sequence corresponds to the sequence AUUAAA. The corresponding positions for each “A” in the polyA signal site sequence (AUUAAA) are denoted as 0, 3, 4, and 5 from left to right. In some cases, editing the polyA signal site sequence can comprise editing the polyA signal site at any A. In some cases, editing can comprise editing from about: 20% to about 95%, 30% to about 95%, 40% to about 95%, 44% to about 91%, 60% to about 95%, or 80% to about 91% of any A position in the polyA tail. In some cases, an engineered guide RNA for targeting the DUX4 polyA signal site sequence at position “0” can comprise a sequence with at least: 70%, 80%, 85%, 90%, 95%, 98%, 99%, or 100% identity and/or length to SEQ ID NO: 1575, 593, 1573, 934, 1569, 1567, 851, 1211, 1571, 937, 1574, 1570, 1566, 1117, 906, 1572, 1104, 352, 512, 1587, 375, 1588, 977, 642, 1236, 1584, 252, 394, 482, 1585, 291, 356, 1054, 1581, 1103, 502, 769, 408, 1586, 1008, 737, 985, 679, 727, 1578, 365, 1580, 487, 1098, or 976. In some cases, editing can comprise editing from about: 20% to about 85%, 30% to about 85%, 40% to about 85%, 50% to about 66%, 40% to about 70%, or 60% to about 66% of the A at position “0” in the polyA tail. In some cases, an engineered guide RNA for targeting the DUX4 polyA signal site sequence at position “3” can comprise a sequence with at least: 70%, 80%, 85%, 90%, 95%, 98%, 99%, or 100% identity and/or length to SEQ ID NO: 1573, 1588, 1545, 1575, 1569, 1584, 1572, 1567, 1570, 1587, 1574, 625, 1571, 874, 17, 1585, 757, 1581, 1538, 8, 1002, 1566, 486, 1552, 505, 635, 606, 884, 1054, 880, 1411, 1568, 871, 1580, 1539, 14, 892, 1116, 15, 1586, 593, 10, 977, 1578, 1579, 747, 1577, 748, 873, or 494. In some cases, editing can comprise editing from about: 20% to about 95%, 30% to about 95%, 40% to about 95%, 76% to about 91%, 60% to about 80%, or 80% to about 91% of the A at position “3” in the polyA tail. In some cases, an engineered guide RNA for targeting the DUX4 polyA signal site sequence at position “4” can comprise a sequence with at least: 70%, 80%, 85%, 90%, 95%, 98%, 99%, or 100% identity and/or length to SEQ ID NO: 1575, 1573, 1567, 1569, 1570, 1566, 1572, 1587, 1571, 1574, 1584, 1588, 1054, 1586, 1585, 1581, 1578, 1580, 934, 72, 1582, 1066, 1183, 1577, 967, 1568, 930, 566, 1463, 1294, 1293, 1391, 1579, 1583, 944, 815, 1168, 593, 594, 694, 1576, 1193, 1051, 1212, 806, 1059, 1374, 195, 358, or 1296. In some cases, editing can comprise editing from about: 20% to about 85%, 30% to about 85%, 40% to about 85%, 54% to about 77%, 50% to about 60%, or 60% to about 77% of the A at position “4” in the polyA tail. In some cases, an engineered guide RNA for targeting the DUX4 polyA signal site sequence at position “5” can comprise a sequence with at least: 70%, 80%, 85%, 90%, 95%, 98%, 99%, or 100% identity and/or length to SEQ ID NO: 1575, 1573, 1569, 1574, 1570, 1572, 1567, 1587, 1566, 1571, 1588, 72, 1586, 1584, 1581, 1578, 1585, 1582, 1580, 1183, 1568, 1066, 1391, 1168, 1293, 1577, 1054, 566, 1579, 930, 694, 944, 195, 1583, 815, 1576, 1051, 1411, 24, 1163, 935, 680, 1212, 594, 1185, 1463, 1058, 810, 392, or 1104. In some cases, editing can comprise editing from about: 20% to about 85%, 30% to about 85%, 40% to about 85%, 44% to about 70%, 50% to about 60%, or 60% to about 70% of the A at position “5” in the polyA tail.

In some embodiments, an engineered guide RNA disclosed herein for targeting DUX4 can comprise a structural feature that is formed in a guide-target RNA scaffold. In some cases, the structural feature comprises a symmetrical internal loop formed by 6 nucleotides on the engineered guide RNA side of the guide-target RNA scaffold target and 6 nucleotides on the target RNA side of the guide-target RNA scaffold. In some cases, the internal loop can start 6 nucleotides upstream (5′) of the target A (0 position) of the target RNA sequence. In some cases, an engineered guide RNA can comprise two or more 6 nucleotide symmetrical internal loops. In some cases, one symmetrical internal loop can be upstream (5′) of the target A (0 position) and one symmetrical internal loop can be downstream (3′) of the target A. In some cases, the structural feature comprises a mismatch formed by 1 nucleotide on the engineered guide RNA side of the guide-target RNA scaffold target and 1 nucleotide on the target RNA side of the guide-target RNA scaffold. In some cases, the mismatch is a A/C mismatch. In some instances, the A/C mismatch comprises the C in an engineered guide RNA of the present disclosure opposite an A in a target RNA. In some cases, the mismatch may be at the target A (0 position) or 3 or 5 nucleotides downstream (3′) from the target A. In some cases, the structural feature comprises a symmetrical bulge formed by 4 nucleotides on the engineered guide RNA side of the guide-target RNA scaffold target and 4 nucleotides on the target RNA side of the guide-target RNA scaffold. In some cases, the structural feature comprises a symmetrical bulge formed by 2 nucleotides on the engineered guide RNA side of the guide-target RNA scaffold target and 2 nucleotides on the target RNA side of the guide-target RNA scaffold. In some instances, a symmetrical bulge is downstream (3′) from the target A.

Assays for Measuring Efficacious Engineered gRNAs Targeting DUX4

In some embodiments, the engineered guide RNAs of the present disclosure facilitate ADAR-mediated RNA editing of DUX4. In some embodiments, ADAR-mediated RNA editing of DUX4 can result in a knockdown (e.g., a reduction) of protein levels, a knockdown in mRNA levels, or both. In some cases, a knockdown of protein levels can be of DUX4 or of a protein downstream of DUX4. In some cases, a knockdown of mRNA levels can be of DUX4 or of a protein downstream of DUX4. In some instances, the knockdown of protein levels and/or mRNA levels is an ADAR dependent knockdown.

In some embodiments, an assay is used to determine the efficacy of a guide RNA disclosed herein. In some cases, an assay can comprise measuring RNA editing, mRNA levels, or protein levels in a cell. In some cases, an assay can comprise measuring RNA editing, mRNA levels, or protein levels in a cell before and after a treatment with a guide RNA disclosed herein. In some cases, cells can be sampled in a time course assay. In some cases, a cell can comprise a cell with a functional ADAR gene. In some cases, a cell can comprise a cell with a nonfunctional ADAR gene. For example, a cell can comprise a truncated or mutated ADAR gene or a cell can comprise a deleted ADAR gene. In some cases, an assay can be used to compare editing levels, levels of mRNA, or levels of protein, in a cell with a functional copy of an ADAR gene and in a cell without a functional ADAR gene. In some cases, the reduction of mRNA or protein levels in the cell can be identified as ADAR dependent reduction in mRNA or protein levels. Protein levels in a cell can be measured by any standard technique, for example a Western Blot. mRNA levels in a cell can be measured by any standard technique, for example by Real-Time Quantitative Reverse Transcription PCR, or droplet digital PCR. In some cases, protein levels can be determined by a functional assay specific to a protein of interest. For example, an assay can be used to determine the amount of a protein by an enzymatic assay measuring the enzyme kinetics of the protein.

In some embodiments, a guide RNA disclosed herein can facilitate ADAR dependent knockdown of mRNA levels or protein levels of 1 to 100%. In some cases, a guide RNA disclosed herein can facilitate ADAR dependent knockdown of mRNA levels or protein levels from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as compared to a cell before treatment with the guide RNA. In some cases, ADAR dependent knockdown of mRNA levels or protein levels can be compared between a cell comprising a functional copy of ADAR and a cell comprising a nonfunctional copy of ADAR.

In some embodiments, the engineered guide RNAs of the present disclosure facilitate ADAR-mediated RNA editing of DUX4-FL, which results in knockdown of protein levels. The knockdown in protein levels is quantitated as a reduction in expression of the DUX4-FL protein. The engineered guide RNAs of the present disclosure can facilitate from 1% to 100% DUX4-FL protein knockdown. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% DUX4-FL protein knockdown. In some embodiments, the engineered guide RNAs of the present disclosure facilitate from 30% to 60% DUX4-FL protein knockdown. Protein knockdown (e.g., DUX4-FL knockdown) can be measured by an assay comparing a sample or subject treated with the engineered guide RNA to a control sample or subject not treated with the engineered guide RNA. In some cases, protein knockdown can be measured by comparing the amount of the protein present in a sample or subject before a treatment with a guide RNA disclosed herein and comparing to the amount of the protein after the treatment.

In some embodiments, ADAR-mediated RNA editing of DUX4-FL, results in knockdown of downstream protein levels of one or more proteins downstream of DUX4. In some instances, a knockdown of a protein downstream of DUX4 can be used to determine the reduction of DUX-4 protein levels. In some cases, a downstream protein of DUX4 comprises SLC34A2, LEUTX, ZSCAN4, PRAMEF12, TRIM43, DEFB103, or MBD3L2. The knockdown in protein levels of a downstream protein of DUX4 can be quantitated as a reduction in expression of the SLC34A2 protein, the LEUTX protein, the ZSCAN4 protein, the PRAMEF12 protein, the TRIM43 protein, the DEFB103 protein, or the MBD3L2 protein. The engineered guide RNAs of the present disclosure can facilitate from 1% to 10%, from 10% to 20%, from 20% to 30%, from 30% to 40%, from 40% to 50%, from 50% to 60%, from 60% to 70%, from 70% to 80%, from 80% to 90%, from 90% to 100%, from 20% to 40%, from 30% to 50%, from 40% to 60%, from 50% to 70%, from 60% to 80%, from 20% to 50%, from 30% to 60%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% protein knockdown of SLC34A2, LEUTX, ZSCAN4, PRAMEF12, TRIM43, DEFB103, MBD3L2 or of another protein downstream of DUX4. In some embodiments, increased editing of the DUX4 RNA by the guide RNA is measured in an assay. In some cases, the increased editing comprises an increase in a protein knockdown of DUX4 and/or of a protein downstream of DUX4. In some cases, the assay can comprise measuring the level of a protein in a sample before and after treatment with a guide RNA described herein. In some cases, the assay can comprise measuring the level of a protein in a sample that is not treated with a guide RNA and measuring the protein in a sample that is treated with a guide RNA described herein.

In some embodiments, the engineered guide RNAs of the present disclosure facilitate ADAR-mediated RNA editing of DUX4, which results in knockdown of mRNA levels. The knockdown in mRNA levels is quantitated as a reduction in expression of the DUX4 mRNA transcript protein. The engineered guide RNAs of the present disclosure can facilitate a 1% to 100% decrease of DUX4 mRNA. The engineered guide RNAs of the present disclosure can facilitate a decrease of: 1% to 10%, 10% to 20%, 20% to 30%, 30% to 40%, 40% to 50%, 50% to 60%, 60% to 70%, 70% to 80%, 80% to 90%, 90% to 100%, 20% to 40%, 30% to 50%, 40% to 60%, 50% to 70%, 60% to 80%, 20% to 50%, or 30% to 60% of DUX4 mRNA. The engineered guide RNAs of the present disclosure can facilitate a decrease of: at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of DUX4 mRNA. The engineered guide RNAs of the present disclosure can facilitate a decrease of at least 50%, or at least 70% of DUX4 mRNA. In some embodiments, the engineered guide RNAs of the present disclosure facilitate a decrease of 50% to 75% of DUX4 mRNA. DUX4 (e.g., DUX4-FL) mRNA levels can be measured by an assay comparing a sample or subject treated with the engineered guide RNA to a control sample or subject not treated with the engineered guide RNA.

In some embodiments, the engineered guide RNAs of the present disclosure facilitate ADAR-mediated RNA editing of DUX4, which results in knockdown of mRNA levels of proteins downstream of DUX4. In some cases, a protein downstream of DUX4 can comprise SLC34A2, LEUTX, ZSCAN4, PRAMEF12, TRIM43, DEFB103, or MBD3L2. In some cases, a reduction in the expression of the mRNA of SLC34A2, LEUTX, ZSCAN4, PRAMEF12, TRIM43, DEFB103, or MBD3L2 can indicate a reduction in the expression of DUX4. The engineered guide RNAs of the present disclosure can facilitate a 1% to 100% decrease of SLC34A2, LEUTX, ZSCAN4, PRAMEF12, TRIM43, DEFB103, or MBD3L2 mRNA. The engineered guide RNAs of the present disclosure can facilitate a decrease of: 1% to 10%, 10% to 20%, 20% to 30%, 30% to 40%, 40% to 50%, 50% to 60%, 60% to 70%, 70% to 80%, 80% to 90%, 90% to 100%, 20% to 40%, 30% to 50%, 40% to 60%, 50% to 70%, 60% to 80%, 20% to 50%, or 30% to 60% of SLC34A2, LEUTX, ZSCAN4, PRAMEF12, TRIM43, DEFB103, or MBD3L2 mRNA. The engineered guide RNAs of the present disclosure can facilitate a decrease of: at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of SLC34A2, LEUTX, ZSCAN4, PRAMEF12, TRIM43, DEFB103, or MBD3L2 mRNA. SLC34A2, LEUTX, ZSCAN4, PRAMEF12, TRIM43, DEFB103, or MBD3L2. mRNA levels can be measured by an assay comparing a sample or subject treated with the engineered guide RNA to a control sample or subject not treated with the engineered guide RNA.

DMPK. The present disclosure provides for engineered guide RNAs that facilitate RNA editing DMPK to knockdown expression of myotonic dystrophy protein kinase. Myotonic dystrophy (DM1) is a rare neuromuscular disease characterized by progressive muscular weakness and an inability to relax muscles (myotonia), predominantly distal skeletal muscles. Genetic causes of DM1 include expansion of CTG repeats in the 3′UTR of the DMPK gene, causing protein aggregates and subsequent muscle wasting. Severity is linked to age of onset and size of the CTG repeat region. Said DMPK mutations are autosomal dominant and is prevalent in 1:2,300 (˜140,000 patients in the US). Target cell types are skeletal and cardiac muscle cells. In some embodiments, the present disclosure provides compositions of engineered guide RNAs that target DMPK and facilitated ADAR-mediated RNA editing of DMPK. In some embodiments, the engineered guide RNAs of the present disclosure target a coding sequence in DMPK. For example, the coding sequence can be a translation initiation site (TIS) (AUG) of DMPK and the engineered guide RNA can facilitate ADAR-mediated RNA editing of AUG to GUG. In some embodiments, the engineered guide RNAs of the present disclosure target a splice site in DMPK pre-mRNA. In some embodiments, the engineered guide RNAs of the present disclosure target a non-coding sequence in DMPK. The non-coding sequence can be a polyA signal sequence and the engineered guide RNA can facilitate ADAR-mediated RNA editing of one or more adenosines in the polyA signal sequence of DMPK. In some embodiments, engineered guide RNAs of the present disclosure can be multiplexed to target more than one polyA signal sequences in DMPK. In some embodiments, engineered guide RNAs of the present disclosure can be multiplexed to target the TIS and one or more polyA signal sequences in DMPK. The engineered guide RNAs of the present disclosure facilitated ADAR-mediated RNA editing of DMPK, thereby, effecting protein knockdown.

PMP22. The present disclosure provides for engineered guide RNAs that facilitate RNA editing of PMP22 to knockdown expression of peripheral myelin protein-22 (PMP22). Charcot-Marie-Tooth Syndrome (CMT1A) is the most common genetically-driven peripheral neuropathy, characterized by progressive distal muscle atrophy, sensory loss and foot/hand deformities. Genetic causes of CMT1A include PMP22 gene duplication leading to peripheral nerve dysmyelination and poor nerve conduction. Said PMP22 mutations are autosomal dominant and prevalence is in 1:7,500 (˜42,000 patients in the US). Target cell types are Schwann cells. In some embodiments, the present disclosure provides compositions of engineered guide RNAs that target PMP22 and facilitated ADAR-mediated RNA editing of PMP22. In some embodiments, the engineered guide RNAs of the present disclosure target a coding sequence in PMP22. For example, the coding sequence can be a translation initiation site (TIS) (AUG) of PMP22 and the engineered guide RNA can facilitate ADAR-mediated RNA editing of AUG to GUG. In some embodiments, the engineered guide RNAs of the present disclosure target a splice site in PMP22 pre-mRNA. In some embodiments, the engineered guide RNAs of the present disclosure target a non-coding sequence in PMP22. The non-coding sequence can be a polyA signal sequence and the engineered guide RNA can facilitate ADAR-mediated RNA editing of one or more adenosines in the polyA signal sequence of PMP22. In some embodiments, engineered guide RNAs of the present disclosure can be multiplexed to target more than one polyA signal sequences in PMP22. In some embodiments, engineered guide RNAs of the present disclosure can be multiplexed to target the TIS and one or more polyA signal sequences in PMP22. The engineered guide RNAs of the present disclosure facilitated ADAR-mediated RNA editing of PMP22, thereby, effecting protein knockdown.

SOD1. The present disclosure provides for engineered guide RNAs that facilitate RNA editing of SOD1 to knockdown expression of the superoxide dismutase enzyme. Amyotrophic lateral sclerosis (ALS) is a rapidly progressing neurodegenerative disease characterized by death of motor neurons and loss of voluntary muscle movement. While the exact cause of ALS is unknown, gain-of-function mutations in SOD1 account for ˜20% of familiar ALS and 2% of spontaneous ALS. Said SOD1 mutations are autosomal dominant and have a prevalence of 2:100,000 (<1,000 patients in US). Target cell types are motor neurons. In some embodiments, the present disclosure provides compositions of engineered guide RNAs that target SOD1 and facilitated ADAR-mediated RNA editing of SOD1. In some embodiments, the engineered guide RNAs of the present disclosure target a coding sequence in SOD1. For example, the coding sequence can be a translation initiation site (TIS) (AUG) of SOD1 and the engineered guide RNA can facilitate ADAR-mediated RNA editing of AUG to GUG. In some embodiments, the engineered guide RNAs of the present disclosure target a splice site in SOD1 pre-mRNA. In some embodiments, the engineered guide RNAs of the present disclosure target a non-coding sequence in SOD1. The non-coding sequence can be a polyA signal sequence and the engineered guide RNA can facilitate ADAR-mediated RNA editing of one or more adenosines in the polyA signal sequence of SOD1. In some embodiments, engineered guide RNAs of the present disclosure can be multiplexed to target more than one polyA signal sequences in SOD1. In some embodiments, engineered guide RNAs of the present disclosure can be multiplexed to target the TIS and one or more polyA signal sequences in SOD1. The engineered guide RNAs of the present disclosure facilitated ADAR-mediated RNA editing of SOD1, thereby, effecting protein knockdown.

An engineered guide RNA of the present disclosure can be used in a method of treating a disorder in a subject in need thereof. For example, an engineered guide RNA disclosed herein can be used to treat facioscapulohumeral muscular dystrophy and/or myotonic dystrophy. A disorder can be a disease, a condition, a genotype, a phenotype, or any state associated with an adverse effect. In some embodiments, treating a disorder can comprise preventing, slowing progression of, reversing, or alleviating symptoms of the disorder. A method of treating a disorder can comprise delivering an engineered polynucleotide encoding an engineered guide RNA to a cell of a subject in need thereof and expressing the engineered guide RNA in the cell. In some embodiments, an engineered guide RNA of the present disclosure can be used to treat a genetic disorder (e.g., FSHD, DM1, CMT1A, or ALS). In some embodiments, an engineered guide RNA disclosed herein can be used to treat FSHD. In some cases, FSHD can comprise FSHD I or FSHD II. In some embodiments, an engineered guide RNA disclosed herein can be used to treat FSHD I. In some embodiments, an engineered guide RNA disclosed herein can be used to treat FSHD II. In some embodiments, an engineered guide RNA of the present disclosure can be used to treat a condition associated with one or more mutations. For example, disclosed herein are methods of treating FSHD with engineered guide RNAs targeting DUX4. Also disclosed herein are methods of treating DM1 with engineered guide RNAs targeting DMPK. Also disclosed herein are methods of treating CMT1A with engineered guide RNAs targeting PMP22. Also disclosed herein are methods of treating ALS with engineered guide RNAs targeting SOD1.

In some embodiments, treatment of FSHD comprises treatment of the symptoms associated with FSHD. A symptom of FSHD can comprise a weakness or atrophy of muscle, such as a muscle of the face, an arm muscle, a neck muscle, a shoulder muscle, a thigh muscle, a hip muscle, an abdominal muscle, a back muscle, a foot muscle, a hand muscle, or any combination thereof. In some cases, a symptom of FSHD can comprise a vision loss, a respiratory insufficiency, a dysphagia, a lordosis, a scoliosis, a hearing loss, a pain, an inflammation (e.g., inflammation of muscles), shoulder weakness, unequal (nonsymmetrical weakness) of the body, or any combination thereof.

### Pharmaceutical Compositions

The compositions described herein (e.g., compositions comprising an engineered guide RNA or an engineered polynucleotide encoding an engineered guide RNA) can be formulated with a pharmaceutically acceptable carrier for administration to a subject (e.g., a human or a non-human animal). The compositions described herein (e.g., compositions comprising an engineered guide RNA or an engineered polynucleotide encoding an engineered guide RNA) can be formulated with a pharmaceutically acceptable: excipient, carrier, diluent or any combination thereof for administration to a subject (e.g., a human or a non-human animal). A pharmaceutically acceptable carrier and/or diluent can include, but is not limited to, phosphate buffered saline solution, water, emulsions (e.g., an oil/water emulsion or a water/oil emulsions), glycerol, liquid polyethylene glycols, aprotic solvents such (e.g., dimethylsulfoxide, N-methylpyrrolidone, or mixtures thereof), and various types of wetting agents, solubilizing agents, anti-oxidants, bulking agents, protein carriers such as albumins, any and all solvents, dispersion media, coatings, sodium lauryl sulfate, isotonic and absorption delaying agents, disintegrants (e.g., potato starch or sodium starch glycolate), and the like. The compositions also can include stabilizers and preservatives. Additional examples of carriers, stabilizers, and adjuvants consistent with the compositions of the present disclosure can be found in, for example, Remington's Pharmaceutical Sciences, 21st Ed., Mack Publ. Co., Easton, Pa. (2005), incorporated herein by reference in its entirety.

### Delivery

An engineered guide RNA of the present disclosure or an engineered polynucleotide of the present disclosure (e.g., an engineered polynucleotide encoding an engineered guide RNA) can be delivered via a delivery vehicle. In some embodiments, the delivery vehicle is a vector. A vector can facilitate delivery of the engineered guide RNA or the engineered polynucleotide into a cell to genetically modify the cell. Target tissues and cells include but are not limited to satellite cells, myoblasts, myocytes, and myotubes of the face, shoulders, and upper limbs. In some examples, the vector comprises DNA, such as double stranded or single stranded DNA. In some examples, the delivery vector can be a eukaryotic vector, a prokaryotic vector (e.g., a bacterial vector or plasmid), a viral vector, or any combination thereof. In some cases, a delivery vehicle can comprise a non-viral delivery vehicle. In some embodiments, the vector is an expression cassette. In some embodiments, a viral vector comprises a viral capsid, an inverted terminal repeat sequence, and the engineered polynucleotide can be used to deliver the engineered guide RNA to a cell.

In some cases, the engineered guide RNA of the present disclosure can be an in vitro transcribed (IVT) RNA. In some cases, an engineered guide RNA can be delivered as a formulation comprising the engineered guide RNA. In some cases, the engineered guide RNA may not be comprised in a vector. In some examples, the engineered guide RNA (e.g., as an oligonucleotide) can be formulated for delivery through direct injection. In some examples, the engineered guide RNA, as an oligo nucleotide can be formulated for delivery through intravenous administration or oral administration.

In some embodiments, the viral vector can be a retroviral vector, an adenoviral vector, an adeno-associated viral (AAV) vector, an alphavirus vector, a lentivirus vector (e.g., human or porcine), a Herpes virus vector, an Epstein-Barr virus vector, an SV40 virus vectors, a pox virus vector, or a combination thereof. In some embodiments, the viral vector can be a recombinant vector, a hybrid vector, a chimeric vector, a self-complementary vector, a single-stranded vector, or any combination thereof.

In some embodiments, the viral vector can be an adeno-associated virus (AAV). In some embodiments, the AAV can be any AAV known in the art. In some embodiments, the AAV can comprise an AAV5 serotype, an AAV6 serotype, an AAV8 serotype, or an AAV9 serotype. In some embodiments, the viral vector can be of a specific serotype. In some embodiments, the viral vector can be an AAV1 serotype, an AAV2 serotype, an AAV3 serotype, an AAV4 serotype, an AAV5 serotype, an AAV6 serotype, an AAV7 serotype, an AAV8 serotype, an AAV9 serotype, an AAV10 serotype, an AAV 11 serotype, an AAV12 serotype, an AAV13 serotype, an AAV14 serotype, an AAV15 serotype, an AAV16 serotype, an AAV.rh8 serotype, an AAV.rh10 serotype, an AAV.rh20 serotype, an AAV.rh39 serotype, an AAV.Rh74 serotype, an AAV.RHM4-1 serotype, an AAV.hu37 serotype, an AAV.Anc80 serotype, an AAV.Anc80L65 serotype, an AAV.7m8 serotype, an AAV.PHP.B serotype, an AAV2.5 serotype, an AAV2tYF serotype, an AAV3B serotype, an AAV.LK03 serotype, an AAV.HSC1 serotype, an AAV.HSC2 serotype, an AAV.HSC3 serotype, an AAV.HSC4 serotype, an AAV.HSC5 serotype, an AAV.HSC6 serotype, an AAV.HSC7 serotype, an AAV.HSC8 serotype, an AAV.HSC9 serotype, an AAV.HSC10 serotype, an AAV.HSC11 serotype, an AAV.HSC12 serotype, an AAV.HSC13 serotype, an AAV.HSC14 serotype, an AAV.HSC15 serotype, an AAV.HSC16 serotype, and an AAVhu68 serotype, a derivative of any of these serotypes, a chimera of any of these serotypes, a variant of any of these serotypes or any combination thereof.

In some embodiments, the AAV vector can be a recombinant vector, a hybrid AAV vector, a chimeric AAV vector, a self-complementary AAV (scAAV) vector, a single-stranded AAV, or any combination thereof.

In some embodiments, the AAV vector can be a recombinant AAV (rAAV) vector. Methods of producing recombinant AAV vectors can be known in the art and generally involve, in some cases, introducing into a producer cell line: (1) DNA necessary for AAV replication and synthesis of an AAV capsid, (b) one or more helper constructs comprising the viral functions missing from the AAV vector, (c) a helper virus, and (d) the plasmid construct containing the genome of the AAV vector, e.g., ITRs, promoter and engineered guide RNA sequences, etc. In some examples, the viral vectors described herein can be engineered through synthetic or other suitable means by references to published sequences, such as those that can be available in the literature. For example, the genomic and protein sequences of various serotypes of AAV, as well as the sequences of the native terminal repeats (TRs), Rep proteins, and capsid subunits can be known in the art and can be found in the literature or in public databases such as GenBank or Protein Data Bank (PDB).

In some examples, methods of producing delivery vectors herein comprising packaging an engineered polynucleotide of the present disclosure (e.g., an engineered polynucleotide encoding an engineered guide RNA) in an AAV vector. In some examples, methods of producing the delivery vectors described herein comprise, (a) introducing into a cell: (i) a polynucleotide comprising a promoter and an engineered guide RNA payload disclosed herein; and (ii) a viral genome comprising a Replication (Rep) gene and Capsid (Cap) gene that encodes a wild-type AAV capsid protein or modified version thereof, (b) expressing in the cell the wild-type AAV capsid protein or modified version thereof; (c) assembling an AAV particle; and (d) packaging the payload disclosed herein in the AAV particle, thereby generating an AAV delivery vector. In some examples, the recombinant vectors comprise one or more inverted terminal repeats and the inverted terminal repeats comprise a 5′ inverted terminal repeat, a 3′ inverted terminal repeat, and a mutated inverted terminal repeat. In some examples, the mutated terminal repeat lacks a terminal resolution site, thereby enabling formation of a self-complementary AAV.

In some examples, a hybrid AAV vector can be produced by transcapsidation, e.g., packaging an inverted terminal repeat (ITR) from a first serotype into a capsid of a second serotype, wherein the first and second serotypes may not be the same. In some examples, the Rep gene and ITR from a first AAV serotype (e.g., AAV2) can be used in a capsid from a second AAV serotype (e.g., AAV5 or AAV9), wherein the first and second AAV serotypes may not be the same. As a non-limiting example, a hybrid AAV serotype comprising the AAV2 ITRs and AAV9 capsid protein can be indicated AAV2/9. In some examples, the hybrid AAV delivery vector comprises an AAV2/1, AAV2/2, AAV 2/4, AAV2/5, AAV2/8, or AAV2/9 vector.

In some examples, the AAV vector can be a chimeric AAV vector. In some examples, the chimeric AAV vector comprises an exogenous amino acid or an amino acid substitution, or capsid proteins from two or more serotypes. In some examples, a chimeric AAV vector can be genetically engineered to increase transduction efficiency, selectivity, or a combination thereof.

In some examples, the AAV vector comprises a self-complementary AAV genome. Self-complementary AAV genomes can be generally known in the art and contain both DNA strands which can anneal together to form double-stranded DNA.

In some examples, the delivery vector can be a retroviral vector. In some examples, the retroviral vector can be a Moloney Murine Leukemia Virus vector, a spleen necrosis virus vector, or a vector derived from the Rous Sarcoma Virus, Harvey Sarcoma Virus, avian leukosis virus, human immunodeficiency virus, myeloproliferative sarcoma virus, or mammary tumor virus, or a combination thereof. In some examples, the retroviral vector can be transfected such that the majority of sequences coding for the structural genes of the virus (e.g., gag, pol, and env) can be deleted and replaced by the gene(s) of interest.

In some examples, the delivery vehicle can be a non-viral vector. In some cases, the delivery vehicle can be a DNA encoding the engineered guide RNA. In some examples, the delivery vehicle can be a plasmid. In some embodiments, the plasmid comprises DNA. In some examples, the plasmid comprises circular double-stranded DNA. In some examples, the plasmid can be linear. In some examples, the plasmid comprises one or more genes of interest and one or more regulatory elements. In some examples, the plasmid comprises a bacterial backbone containing an origin of replication and an antibiotic resistance gene or other selectable marker for plasmid amplification in bacteria. In some examples, the plasmid can be a minicircle plasmid. In some examples, the plasmid contains one or more genes that provide a selective marker to induce a target cell to retain the plasmid. In some examples, the plasmid can be formulated for delivery through injection by a needle carrying syringe. In some examples, the plasmid can be formulated for delivery via electroporation. In some examples, the plasmids can be engineered through synthetic or other suitable means known in the art. For example, in some cases, the genetic elements can be assembled by restriction digest of the desired genetic sequence from a donor plasmid or organism to produce ends of the DNA which can then be readily ligated to another genetic sequence.

In some embodiments, the vector containing the engineered guide RNA or the engineered polynucleotide is a non-viral vector system. In some embodiments, the non-viral vector system comprises cationic lipids, or polymers. For example, the non-viral vector system can be a liposome or polymeric nanoparticle. In some cases, a non-viral vector system can be a lipid nanoparticle (LNP) or a polymer nanoparticle. In some embodiments, the engineered polynucleotide or a non-viral vector comprising the engineered guide RNA or the engineered polynucleotide is delivered to a cell by hydrodynamic injection or ultrasound.

### Administration

Administration can refer to methods that can be used to enable the delivery of a composition described herein (e.g. comprising an engineered guide RNA or an engineered polynucleotide encoding the same) to the desired site of biological action. For example, an engineered guide RNA can be comprised in a DNA construct, a viral vector, or both and be administered by intravenous administration. Administration disclosed herein to an area in need of treatment or therapy can be achieved by, for example, and not by way of limitation, oral administration, topical administration, intravenous administration, inhalation administration, or any combination thereof. In some cases, administration disclosed herein can be a systemic administration. In some instances, administration can be systemic administration by an injection (e.g., intravenous administration or any administration by an injection) or oral delivery. In some embodiments, delivery can include inhalation, otic, buccal, conjunctival, dental, endocervical, endosinusial, endotracheal, enteral, epidural, extra-amniotic, extracorporeal, hemodialysis, infiltration, interstitial, intraabdominal, intraamniotic, intraarterial, intraarticular, intrabiliary, intrabronchial, intrabursal, intracardiac, intracartilaginous, intracaudal, intracavernous, intracavitary, intracerebroventricular, intracisternal, intracorneal, intracoronal, intracoronary, intracorpous cavernaosum, intradermal, intradiscal, intraductal, intraduodenal, intradural, intraepidermal, intraesophageal, intragastric, intragingival, intrahippocampal, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathoracic, intratubular, intratumor, intratympanic, intrauterine, intravascular, intravenous, intravenous bolus, intravenous drip, intravesical, intravitreal, iontophoresis, irrigation, laryngeal, nasal, nasogastric, ophthalmic, oral, oropharyngeal, parenteral, percutaneous, periarticular, peridural, perineural, periodontal, rectal, retrobulbar, subarachnoid, subconjunctival, subcutaneous, sublingual, submucosal, topical, transdermal, transmucosal, transplacental, transtracheal, transtympanic, ureteral, urethral, vaginal, infraorbital, intraparenchymal, intrathecal, intraventricular, stereotactic, or any combination thereof. Delivery can include parenteral administration (including intravenous, subcutaneous, intrathecal, intraperitoneal, intramuscular, intravascular or infusion), oral administration, inhalation administration, intraduodenal administration, rectal administration, or a combination thereof. Delivery can include direct application to the affected tissue or region of the body. In some cases, topical administration can comprise administering a lotion, a solution, an emulsion, a cream, a balm, an oil, a paste, a stick, an aerosol, a foam, a jelly, a foam, a mask, a pad, a powder, a solid, a tincture, a butter, a patch, a gel, a spray, a drip, a liquid formulation, an ointment to an external surface of a surface, such as a skin. Delivery can include a parenchymal injection, an intra-thecal injection, an intra-ventricular injection, or an intra-cisternal injection. A composition provided herein can be administered by any method. A method of administration can be by intra-arterial injection, intracisternal injection, intramuscular injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a liposome, an exosome, an extracellular vesicle, an implant, or a combination thereof. In some cases, delivery can be from a device. In some instances, delivery can be administered by a pump, an infusion pump, or a combination thereof. In some embodiments, delivery can be by an enema, an eye drop, a nasal spray, or any combination thereof. In some instances, a subject can administer the composition in the absence of supervision. In some instances, a subject can administer the composition under the supervision of a medical professional (e.g., a physician, nurse, physician's assistant, orderly, hospice worker, etc.). In some embodiments, a medical professional can administer the composition.

In some cases, administering can be oral ingestion. In some cases, delivery can be a capsule or a tablet. Oral ingestion delivery can comprise a tea, an elixir, a food, a drink, a beverage, a syrup, a liquid, a gel, a capsule, a tablet, an oil, a tincture, or any combination thereof. In some embodiments, a food can be a medical food. In some instances, a capsule can comprise hydroxymethylcellulose. In some embodiments, a capsule can comprise a gelatin, hydroxypropylmethyl cellulose, pullulan, or any combination thereof. In some cases, capsules can comprise a coating, for example, an enteric coating. In some embodiments, a capsule can comprise a vegetarian product or a vegan product such as a hypromellose capsule. In some embodiments, delivery can comprise inhalation by an inhaler, a diffuser, a nebulizer, a vaporizer, or a combination thereof.

In some embodiments, an engineered guide RNA disclosed herein or a polynucleotide encoding the engineered guide RNA can be administered with a second therapeutic. In some cases, the second therapeutic can be administered in an amount sufficient to treat a disease or condition. In some cases, administration of the second therapeutic can be concurrent administration or consecutive administration to administration of the engineered guide RNA disclosed herein or the polynucleotide encoding the engineered guide RNA. In some cases, the second therapeutic can comprise losmapimod or a salt thereof. In some cases, losmapimod or a salt thereof can be administered in an amount of about: 0.0001 gram to about 100 grams or about 1 mg to about 100 mg.

In some embodiments, disclosed herein can be a method, comprising administering a composition disclosed herein to a subject (e.g., a human) in need thereof. In some instances, the method can treat or prevent a disease in the subject.

### Definitions

Unless defined otherwise, all terms of art, notations and other technical and scientific terms or terminology used herein are intended to have the same meaning as commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments are presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term “about” a number can refer to that number plus or minus 10% of that number.

As disclosed herein, a “bulge” refers to the structure substantially formed only upon formation of the guide-target RNA scaffold, where contiguous nucleotides in either the engineered guide RNA or the target RNA are not complementary to their positional counterparts on the opposite strand. A bulge can independently have from 0 to 4 contiguous nucleotides on the guide RNA side of the guide-target RNA scaffold and 1 to 4 contiguous nucleotides on the target RNA side of the guide-target RNA scaffold or a bulge can independently have from 0 to 4 nucleotides on the target RNA side of the guide-target RNA scaffold and 1 to 4 contiguous nucleotides on the guide RNA side of the guide-target RNA scaffold. However, a bulge, as used herein, does not refer to a structure where a single participating nucleotide of the engineered guide RNA and a single participating nucleotide of the target RNA do not base pair—a single participating nucleotide of the engineered guide RNA and a single participating nucleotide of the target RNA that do not base pair is referred to herein as a “mismatch.” Further, where the number of participating nucleotides on either the guide RNA side or the target RNA side exceeds 4, the resulting structure is no longer considered a bulge, but rather, is considered an “internal loop.” A “symmetrical bulge” refers to a bulge where the same number of nucleotides is present on each side of the bulge. An “asymmetrical bulge” refers to a bulge where a different number of nucleotides are present on each side of the bulge.

The term “complementary” or “complementarity” refers to the ability of a nucleic acid to form one or more bonds with a corresponding nucleic acid sequence by, for example, hydrogen bonding (e.g., traditional Watson-Crick), covalent bonding, or other similar methods. In Watson-Crick base pairing, a double hydrogen bond forms between nucleobases T and A, whereas a triple hydrogen bond forms between nucleobases C and G. For example, the sequence A-G-T can be complementary to the sequence T-C-A. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary, respectively). “Perfectly complementary” can mean that all the contiguous residues of a nucleic acid sequence will hydrogen bond with the same number of contiguous residues in a second nucleic acid sequence. “Substantially complementary” as used herein can refer to a degree of complementarity that can be at least 60%, 65%, 70%, 75%, 80%, 85%, 90%, 95%. 97%, 98%, 99%, or 100% over a region of 10, 15, 20, 25, 30, 35, 40, 45, 50, or more nucleotides, or can refer to two nucleic acids that hybridize under stringent conditions (i.e., stringent hybridization conditions). Nucleic acids can include nonspecific sequences. As used herein, the term “nonspecific sequence” or “not specific” can refer to a nucleic acid sequence that contains a series of residues that may not be designed to be complementary to or can be only partially complementary to any other nucleic acid sequence.

The terms “determining,” “measuring,” “evaluating,” “assessing,” “assaying,” and “analyzing” can be used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The term “encode,” as used herein, refers to an ability of a polynucleotide to provide information or instructions sequence sufficient to produce a corresponding gene expression product. In a non-limiting example, mRNA can encode a polypeptide during translation, whereas DNA can encode an mRNA molecule during transcription.

An “engineered latent guide RNA” refers to an engineered guide RNA that comprises a portion of sequence that, upon hybridization or only upon hybridization to a target RNA, substantially forms at least a portion of a structural feature, other than a single A/C mismatch feature at the target adenosine to be edited.

As used herein, the term “facilitates RNA editing” by an engineered guide RNA refers to the ability of the engineered guide RNA when associated with an RNA editing entity and a target RNA to provide a targeted edit of the target RNA by the RNA edited entity. In some instances, the engineered guide RNA can directly recruit or position/orient the RNA editing entity to the proper location for editing of the target RNA. In other instances, the engineered guide RNA when hybridized to the target RNA forms a guide-target RNA scaffold with one or more structural features as described herein, where the guide-target RNA scaffold with structural features recruits or positions/orients the RNA editing entity to the proper location for editing of the target RNA.

A “guide-target RNA scaffold,” as disclosed herein, is the resulting double stranded RNA formed upon hybridization of a guide RNA, with latent structure, to a target RNA. A guide-target RNA scaffold has one or more structural features formed within the double stranded RNA duplex upon hybridization. For example, the guide-target RNA scaffold can have one or more structural features selected from a bulge, mismatch, internal loop, hairpin, or wobble base pair.

As disclosed herein, a “hairpin” includes an RNA duplex wherein a portion of a single RNA strand has folded in upon itself to form the RNA duplex. The portion of the single RNA strand folds upon itself due to having nucleotide sequences that base pair to each other, where the nucleotide sequences are separated by an intervening sequence that does not base pair with itself, thus forming a base-paired portion and non-base paired, intervening loop portion.

As used herein, the term percent “identity,” in the context of two or more nucleic acid or polypeptide sequences, can refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

For purposes herein, percent identity and sequence similarity can be performed using the BLAST algorithm, which is described in Altschul et al. (J. Mol. Biol. 215:403-410 (1990)). Software for performing BLAST analyses is publicly available through the National Center for Biotechnology Information.

As disclosed herein, an “internal loop” refers to the structure substantially formed only upon formation of the guide-target RNA scaffold, where nucleotides in either the engineered guide RNA or the target RNA are not complementary to their positional counterparts on the opposite strand and where one side of the internal loop, either on the target RNA side or the engineered guide RNA side of the guide-target RNA scaffold, has 5 nucleotides or more. Where the number of participating nucleotides on both the guide RNA side and the target RNA side drops below 5, the resulting structure is no longer considered an internal loop, but rather, is considered a “bulge” or a “mismatch,” depending on the size of the structural feature. A “symmetrical internal loop” is formed when the same number of nucleotides is present on each side of the internal loop. An “asymmetrical internal loop” is formed when a different number of nucleotides is present on each side of the internal loop.

“Latent structure” refers to a structural feature that substantially forms only upon hybridization of a guide RNA to a target RNA. For example, the sequence of a guide RNA provides one or more structural features, but these structural features substantially form only upon hybridization to the target RNA, and thus the one or more latent structural features manifest as structural features upon hybridization to the target RNA. Upon hybridization of the guide RNA to the target RNA, the structural feature is formed and the latent structure provided in the guide RNA is, thus, unmasked.

“Messenger RNA” or “mRNA” are RNA molecules comprising a sequence that encodes a polypeptide or protein. In general, RNA can be transcribed from DNA. In some cases, precursor mRNA containing non-protein coding regions in the sequence can be transcribed from DNA and then processed to remove all or a portion of the non-coding regions (introns) to produce mature mRNA. As used herein, the term “pre-mRNA” can refer to the RNA molecule transcribed from DNA before undergoing processing to remove the non-protein coding regions.

As disclosed herein, a “mismatch” refers to a single nucleotide in a guide RNA that is unpaired to an opposing single nucleotide in a target RNA within the guide-target RNA scaffold. A mismatch can comprise any two single nucleotides that do not base pair. Where the number of participating nucleotides on the guide RNA side and the target RNA side exceeds 1, the resulting structure is no longer considered a mismatch, but rather, is considered a “bulge” or an “internal loop,” depending on the size of the structural feature.

As used herein, the term “polynucleotide” can refer to a single or double-stranded polymer of deoxyribonucleotide (DNA) or ribonucleotide (RNA) bases read from the 5′ to the 3′ end. The term “RNA” is inclusive of dsRNA (double stranded RNA), snRNA (small nuclear RNA), lncRNA (long non-coding RNA), mRNA (messenger RNA), miRNA (microRNA) RNAi (inhibitory RNA), siRNA (small interfering RNA), shRNA (short hairpin RNA), tRNA (transfer RNA), rRNA (ribosomal RNA), snoRNA (small nucleolar RNA), and cRNA (complementary RNA). The term DNA is inclusive of cDNA, genomic DNA, and DNA-RNA hybrids.

The term “protein”, “peptide” and “polypeptide” can be used interchangeably and in their broadest sense can refer to a compound of two or more subunit amino acids, amino acid analogs or peptidomimetics. The subunits can be linked by peptide bonds. In another embodiment, the subunit can be linked by other bonds, e.g., ester, ether, etc. A protein or peptide can contain at least two amino acids and no limitation can be placed on the maximum number of amino acids which can comprise a protein's or peptide's sequence. As used herein the term “amino acid” can refer to either natural amino acids, unnatural amino acids, or synthetic amino acids, including glycine and both the D and L optical isomers, amino acid analogs and peptidomimetics. As used herein, the term “fusion protein” can refer to a protein comprised of domains from more than one naturally occurring or recombinantly produced protein, where generally each domain serves a different function. In this regard, the term “linker” can refer to a protein fragment that can be used to link these domains together —optionally to preserve the conformation of the fused protein domains, prevent unfavorable interactions between the fused protein domains which can compromise their respective functions, or both.

The term “structured motif” refers to a combination of two or more structural features in a guide-target RNA scaffold.

The terms “subject,” “individual,” or “patient” can be used interchangeably herein. A “subject” refers to a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject can be diagnosed or suspected of being at high risk for a disease. In some cases, the subject is not necessarily diagnosed or suspected of being at high risk for the disease

The term “in vivo” refers to an event that takes place in a subject's body.

The term “ex vivo” refers to an event that takes place outside of a subject's body. An ex vivo assay may not be performed on a subject. Rather, it can be performed upon a sample separate from a subject. An example of an ex vivo assay performed on a sample can be an “in vitro” assay.

The term “in vitro” refers to an event that takes places contained in a container for holding laboratory reagent such that it can be separated from the biological source from which the material can be obtained. In vitro assays can encompass cell-based assays in which living or dead cells can be employed. In vitro assays can also encompass a cell-free assay in which no intact cells can be employed.

The term “wobble base pair” refers to two bases that weakly pair. For example, a wobble base pair can refer to a G paired with a U.

The term “substantially forms” as described herein, when referring to a particular secondary structure, refers to formation of at least 80% of the structure under physiological conditions (e.g. physiological pH, physiological temperature, physiological salt concentration, etc.).

As used herein, the terms “treatment” or “treating” can be used in reference to a pharmaceutical or other intervention regimen for obtaining beneficial or desired results in the recipient. Beneficial or desired results include but are not limited to a therapeutic benefit and/or a prophylactic benefit. A therapeutic benefit can refer to eradication or amelioration of one or more symptoms of an underlying disorder being treated. Also, a therapeutic benefit can be achieved with the eradication or amelioration of one or more of the physiological symptoms associated with the underlying disorder such that an improvement can be observed in the subject, notwithstanding that the subject can still be afflicted with the underlying disorder. A prophylactic effect includes delaying, preventing, or eliminating the appearance of a disease or condition, delaying or eliminating the onset of one or more symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease can undergo treatment, even though a diagnosis of this disease may not have been made.

### NUMBERED EMBODIMENTS

A number of compositions, and methods are disclosed herein. Specific exemplary embodiments of these compositions and methods are disclosed below. The following embodiments recite non-limiting permutations of combinations of features disclosed herein. Other permutations of combinations of features are also contemplated. In particular, each of these numbered embodiments is contemplated as depending from or relating to every previous or subsequent numbered embodiment, independent of their order as listed.

### Embodiments Section 1

Embodiment 1. A composition comprising an engineered guide RNA, wherein:
a) the engineered guide RNA, upon hybridization to a sequence of a target RNA, forms a guide-target RNA scaffold with the sequence of the target RNA;
b) formation of the guide-target RNA scaffold substantially forms one or more structural features selected from the group consisting of: a bulge, an internal loop, and a hairpin; and
c) the sequence of the target RNA is a sequence of the target RNA is selected from the group consisting of: a translation initiation site, a polyA signal sequence, and any combination thereof.

Embodiment 2. The composition of embodiment 1, wherein the sequence of the target RNA comprises the translation initiation site.

Embodiment 3. The composition of embodiment 1, wherein the sequence of the target RNA comprises the polyA signal site.

Embodiment 4. The composition of any one of embodiments 1-2, wherein upon hybridization of the engineered guide RNA to the sequence of the target RNA, the engineered guide RNA facilitates RNA editing of one or more adenosines in the sequence of the target RNA by an RNA editing entity.

Embodiment 5. The composition of any one of embodiments 1-4, wherein the target RNA is selected from the group consisting of DUX4, DMPK, PMP22, and SOD1.

Embodiment 6. The composition of any one of embodiments 1-4, wherein the target RNA comprises DUX4-FL.

Embodiment 7. The composition of embodiment 6, wherein the sequence of the target RNA comprises the polyA signal sequence, wherein the polyA signal sequence is in DUX4-FL.

Embodiment 8. The composition of embodiment 7, wherein the polyA signal sequence comprises ATTAAA.

Embodiment 9. The composition of embodiment 8, wherein one or more adenosines in the polyA signal sequence of ATTAAA is edited by the RNA editing entity.

Embodiment 10. The composition of any one of embodiments 1-9, wherein the one or more structural features comprises the bulge, wherein the bulge is a symmetric bulge.

Embodiment 11. The composition of any one of embodiments 1-9, wherein the one or more structural features comprises the bulge, wherein the bulge is an asymmetric bulge.

Embodiment 12. The composition of any one of embodiments 1-11, wherein the one or more structural features comprises the internal loop, wherein the internal loop is a symmetric internal loop.

Embodiment 13. The composition of any one of embodiments 1-11, wherein the one or more structural features comprises the internal loop, wherein the internal loop is an asymmetric internal loop.

Embodiment 14. The composition of any one of embodiments 1-13, wherein the guide-target RNA scaffold comprises a Wobble base pair.

Embodiment 15. The composition of any one of embodiments 1-14, wherein the one or more structural features comprises the hairpin, wherein the hairpin is a recruitment hairpin or a non-recruitment hairpin.

Embodiment 16. The composition of embodiment 4, wherein the RNA editing entity comprises ADAR1, ADAR2, ADAR3, or any combination thereof.

Embodiment 17. The composition of any one of embodiments 1-16, wherein the engineered guide RNA is encoded by an engineered polynucleotide.

Embodiment 18. The composition of embodiment 17, wherein the engineered polynucleotide is comprised in or on a vector.

Embodiment 19. The composition of embodiment 18, wherein the vector is a viral vector, and wherein the engineered polynucleotide is encapsidated in the viral vector.

Embodiment 20. The composition of embodiment 19, wherein the viral vector is an adeno-associated viral (AAV) vector or a derivative thereof.

Embodiment 21. The composition of embodiment 20, wherein the AAV vector is AAV1, AAV2, AAV3, AAV4, AAV5, AAV6, AAV7, AAV8, AAV9, AAV10, AAV 11, or a derivative, a chimera, or a variant thereof.

Embodiment 22. The composition of any one of embodiments 20-21, wherein the AAV vector is a recombinant AAV (rAAV) vector, a hybrid AAV vector, a chimeric AAV vector, a self-complementary AAV (scAAV) vector, or any combination thereof

Embodiment 23. The composition of any one of embodiments 1-22, wherein the engineered guide RNA has at least 80%, 85%, 90%, 92%, 95%, 97%, or 99% sequence identity to any one of SEQ ID NO: 2-SEQ ID NO: 1589.

Embodiment 24. The composition of any one of embodiments 1-22, wherein the engineered guide RNA has a sequence of any one of SEQ ID NO: 2-SEQ ID NO: 1589.

Embodiment 25. A pharmaceutical composition comprising:
(a) the composition of any one of embodiments 1-24; and
(b) a pharmaceutically acceptable: excipient, carrier, or diluent.

Embodiment 26. A method of treating a disease or a condition in a subject in need thereof, the method comprising administering to the subject a therapeutically effective amount of the composition of any one of embodiments 1-24 or the pharmaceutical composition of embodiment 25.

Embodiment 27. The method of embodiment 26, wherein the disease or condition comprises facioscapulohumeral muscular dystrophy and the target RNA is DUX4.

Embodiment 28. The method of embodiment 26, wherein the disease or condition comprises myotonic dystrophy and the target RNA is DMPK.

Embodiment 29. The method of embodiment 26, wherein the disease or condition comprises Charcot-Marie-Tooth Syndrome and the target RNA is PMP22.

Embodiment 30. The method of embodiment 26, wherein the disease or condition comprises amyotrophic lateral sclerosis and the target RNA is SOD1.

Embodiments section 2:

1. A composition comprising an engineered guide RNA or an engineered polynucleotide encoding the engineered guide RNA, wherein: a) the engineered guide RNA, upon hybridization to a sequence of a DUX4 target RNA, forms a guide-target RNA scaffold with the sequence of the DUX4 target RNA; b) formation of the guide-target RNA scaffold substantially forms one or more structural features selected from the group consisting of: a bulge, an internal loop, a hairpin, and a mismatch formed by a base in the engineered guide RNA to a G, a C, or a U in the DUX4 target RNA; and c) the structural feature is not present within the engineered guide RNA prior to the hybridization of the engineered guide RNA to the DUX4 target RNA; and d) upon hybridization of the engineered guide RNA to the sequence of the DUX4 target RNA, the engineered guide RNA facilitates RNA editing of one or more target adenosines in the sequence of the DUX4 target RNA by an RNA editing entity. 2. The composition of embodiment 1, wherein the sequence of the DUX4 target RNA comprises a translation initiation site, a polyA signal sequence, a splice site, or any combination thereof. 3. The composition of embodiment 2, wherein the sequence of the DUX4 target RNA comprises the translation initiation site. 4. The composition of embodiment 2, wherein the sequence of the DUX4 target RNA comprises the polyA signal sequence. 5. The composition of embodiment 1, wherein the one or more features further comprises a mismatch formed by a base in the engineered guide RNA to an A in the DUX4 target RNA. 6. The composition of embodiment 1, wherein the DUX4 is DUX4-FL. 7. The composition of embodiment 6, wherein the sequence of the DUX4 target RNA comprises the polyA signal sequence, wherein the polyA signal sequence is in DUX4-FL. 8. The composition of embodiment 7, wherein the polyA signal sequence comprises ATTAAA. 9. The composition of embodiment 8, wherein any A of the ATTAAA polyA signal sequence is the target adenosine. 10. The composition of any one of embodiments 6-9, wherein position 0 of ATTAAA is the target adenosine, wherein position 0 is the first A of ATTAAA at the 5′ end. 11. The composition of embodiment 10, wherein the one or more structural features comprises: a first 6/6 symmetric internal loop at a position selected from the group consisting of: −3, −4, −5, −6, −7, −8, −9, −10, and −11, relative to position 0 of ATTAAA. 12. The composition of embodiment 11, wherein the first 6/6 symmetric internal loop is at position −3 relative to position 0. 13. The composition of embodiment 12, wherein the one or more structural features further comprises at least one structural feature selected from the group consisting of: an A/C mismatch at position 0, a second 6/6 symmetric internal loop at position 24 relative to position 0, and a combination thereof 14. The composition of embodiment 13, wherein the engineered guide RNA comprises at least about: 80%, 85%, 90%, 92%, 95%, 97%, or 99% sequence identity to SEQ ID NO: 1236. 15. The composition of embodiment 14, wherein the engineered guide RNA comprises SEQ ID NO: 1236. 16. The composition of embodiment 11, wherein the first 6/6 symmetric internal loop is at position −4 relative to position 0. 17. The composition of embodiment 16, wherein the one or more structural features further comprises at least one structural feature selected from the group consisting of: an A/C mismatch at position 0, a second 6/6 symmetric internal loop at position 42 relative to position 0, and a combination thereof. 18. The composition of embodiment 17, wherein the engineered guide RNA comprises at least about: 80%, 85%, 90%, 92%, 95%, 97%, or 99% sequence identity to SEQ ID NO: 1211. 19. The composition of embodiment 18, wherein the engineered guide RNA comprises SEQ ID NO: 1211. 20. The composition of embodiment 16, wherein the one or more structural features further comprises at least one structural feature selected from the group consisting of: an A/C mismatch at position 0, a second 6/6 symmetric internal loop at position 23 relative to position 0, and a combination thereof. 21. The composition of embodiment 20, wherein the engineered guide RNA comprises at least about: 80%, 85%, 90%, 92%, 95%, 97%, or 99% sequence identity to SEQ ID NO: 1117. 22. The composition of embodiment 21, wherein the engineered guide RNA comprises SEQ ID NO: 1117. 23. The composition of embodiment 11, wherein the first 6/6 symmetric internal loop is at position −5 relative to position 0. 24. The composition of embodiment 23, wherein the one or more structural features further comprises at least one structural feature selected from the group consisting of: an A/C mismatch at position 0, a second 6/6 symmetric internal loop at position 24 relative to position 0, and a combination thereof. 25. The composition of embodiment 24, wherein the engineered guide RNA comprises at least about: 80%, 85%, 90%, 92%, 95%, 97%, or 99% sequence identity to SEQ ID NO: 1008. 26. The composition of embodiment 25, wherein the engineered guide RNA comprises SEQ ID NO: 1008. 27. The composition of embodiment 23, wherein the one or more structural features further comprises a second 6/6 symmetric internal loop at position 33 relative to position 0. 28. The composition of embodiment 27, wherein the engineered guide RNA comprises at least about: 80%, 85%, 90%, 92%, 95%, 97%, or 99% sequence identity to SEQ ID NO: 1054. 29. The composition of embodiment 28, wherein the engineered guide RNA comprises SEQ ID NO: 1054. 30. The composition of embodiment 23, wherein the one or more structural features further comprises at least one structural feature selected from the group consisting of: an A/C mismatch at position 0, a second 6/6 symmetric internal loop at position 44 relative to position 0, and a combination thereof 31. The composition of embodiment 30, wherein the engineered guide RNA comprises at least about: 80%, 85%, 90%, 92%, 95%, 97%, or 99% sequence identity to SEQ ID NO: 1103. 32. The composition of embodiment 31, wherein the engineered guide RNA comprises SEQ ID NO: 1103. 33. The composition of embodiment 23, wherein the one or more structural features further comprises at least one structural feature selected from the group consisting of: an A/C mismatch at position 0, a second 6/6 symmetric internal loop at position 43 relative to position 0, and a combination thereof. 34. The composition of embodiment 33, wherein the engineered guide RNA comprises at least about: 80%, 85%, 90%, 92%, 95%, 97%, or 99% sequence identity to SEQ ID NO: 1098. 35. The composition of embodiment 34, wherein the engineered guide RNA comprises SEQ ID NO: 1098. 36. The composition of embodiment 23, wherein the one or more structural features further comprises at least one structural feature selected from the group consisting of: an A/C mismatch at position 3 relative to position 0, a second 6/6 symmetric internal loop at position 44 relative to position 0, and a combination thereof. 37. The composition of embodiment 36, wherein the engineered guide RNA comprises at least about: 80%, 85%, 90%, 92%, 95%, 97%, or 99% sequence identity to SEQ ID NO: 1104. 38. The composition of embodiment 37, wherein the engineered guide RNA comprises SEQ ID NO: 1104. 39. The composition of embodiment 11, wherein the first 6/6 symmetric internal loop is at position −6 relative to position 0. 40. The composition of embodiment 39, wherein the one or more structural features further comprises at least one structural feature selected from the group consisting of: an A/C mismatch at position 3 relative to position 0, a second 6/6 symmetric internal loop at position 42 relative to position 0, and a combination thereof. 41. The composition of embodiment 40, wherein the engineered guide RNA comprises at least about: 80%, 85%, 90%, 92%, 95%, 97%, or 99% sequence identity to SEQ ID NO: 977. 42. The composition of embodiment 41, wherein the engineered guide RNA comprises SEQ ID NO: 977. 43. The composition of embodiment 39, wherein the one or more structural features further comprises at least one structural feature selected from the group consisting of: an A/C mismatch at position 0, a second 6/6 symmetric internal loop at position 27 relative to position 0, and a combination thereof. 44. The composition of embodiment 43, wherein the engineered guide RNA comprises at least about: 80%, 85%, 90%, 92%, 95%, 97%, or 99% sequence identity to SEQ ID NO: 906. 45. The composition of embodiment 44, wherein the engineered guide RNA comprises SEQ ID NO: 906. 46. The composition of embodiment 39, wherein the one or more structural features further comprises at least one structural feature selected from the group consisting of: an A/C mismatch at position 0, a second 6/6 symmetric internal loop at position 40 relative to position 0, and a combination thereof. 47. The composition of embodiment 46, wherein the engineered guide RNA comprises at least about: 80%, 85%, 90%, 92%, 95%, 97%, or 99% sequence identity to SEQ ID NO: 937. 48. The composition of embodiment 47, wherein the engineered guide RNA comprises SEQ ID NO: 937. 49. The composition of embodiment 39, wherein the one or more structural features further comprises at least one structural feature selected from the group consisting of: an A/C mismatch at position 0, a second 6/6 symmetric internal loop at position 33 relative to position 0, and a combination thereof 50. The composition of embodiment 49, wherein the engineered guide RNA comprises at least about: 80%, 85%, 90%, 92%, 95%, 97%, or 99% sequence identity to SEQ ID NO: 934. 51. The composition of embodiment 50, wherein the engineered guide RNA comprises SEQ ID NO: 934. 52. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 53 relative to position 0, a 4/4 symmetric bulge at position 71 relative to position 0, and any combination thereof 53. The composition of embodiment 52, wherein the engineered guide RNA comprises at least about: 80%, 85%, 90%, 92%, 95%, 97%, or 99% sequence identity to SEQ ID NO: 1584. 54. The composition of embodiment 53, wherein the engineered guide RNA comprises SEQ ID NO: 1584. 55. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 5/5 internal loop at position 53 relative to position 0, a 5/5 internal loop at position 72 relative to position 0, and any combination thereof. 56. The composition of embodiment 55, wherein the engineered guide RNA comprises at least about: 80%, 85%, 90%, 92%, 95%, 97%, or 99% sequence identity to SEQ ID NO: 1585. 57. The composition of embodiment 56, wherein the engineered guide RNA comprises SEQ ID NO: 1585. 58. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 5/5 internal loop at position 51 relative to position 0, a 5/5 internal loop at position 68 relative to position 0, and any combination thereof. 59. The composition of embodiment 58, wherein the engineered guide RNA comprises at least about: 80%, 85%, 90%, 92%, 95%, 97%, or 99% sequence identity to SEQ ID NO: 1581. 60. The composition of embodiment 59, wherein the engineered guide RNA comprises SEQ ID NO: 1581. 61. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 51 relative to position 0, a 2/2 symmetric bulge at position 65 relative to position 0, and any combination thereof 62. The composition of embodiment 61, wherein the engineered guide RNA comprises at least about: 80%, 85%, 90%, 92%, 95%, 97%, or 99% sequence identity to SEQ ID NO: 1578. 63. The composition of embodiment 62, wherein the engineered guide RNA comprises SEQ ID NO: 1578. 64. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof. 65. The composition of embodiment 64, wherein the engineered guide RNA comprises at least about: 80%, 85%, 90%, 92%, 95%, 97%, or 99% sequence identity to SEQ ID NO: 1575. 66. The composition of embodiment 65, wherein the engineered guide RNA comprises SEQ ID NO: 1575. 67. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 5/5 internal loop at position 47 relative to position 0, a 5/5 internal loop at position 60 relative to position 0, a 5/5 internal loop at position 73 relative to position 0, and any combination thereof. 68. The composition of embodiment 67, wherein the engineered guide RNA comprises at least about: 80%, 85%, 90%, 92%, 95%, 97%, or 99% sequence identity to SEQ ID NO: 1573. 69. The composition of embodiment 68, wherein the engineered guide RNA comprises SEQ ID NO: 1573. 70. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 5/5 internal loop at position 45 relative to position 0, a 5/5 internal loop at position 56 relative to position 0, a 5/5 internal loop at position 67 relative to position 0, and any combination thereof. 71. The composition of embodiment 70, wherein the engineered guide RNA comprises at least about: 80%, 85%, 90%, 92%, 95%, 97%, or 99% sequence identity to SEQ ID NO: 1569. 72. The composition of embodiment 71, wherein the engineered guide RNA comprises SEQ ID NO: 1569. 73. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof. 74. The composition of embodiment 73, wherein the engineered guide RNA comprises at least about: 80%, 85%, 90%, 92%, 95%, 97%, or 99% sequence identity to SEQ ID NO: 1567. 75. The composition of embodiment 74, wherein the engineered guide RNA comprises SEQ ID NO: 1567. 76. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 47 relative to position 0, a 3/3 symmetric bulge at position 58 relative to position 0, a 3/3 symmetric bulge at position 69 relative to position 0, and any combination thereof. 77. The composition of embodiment 76, wherein the engineered guide RNA comprises at least about: 80%, 85%, 90%, 92%, 95%, 97%, or 99% sequence identity to SEQ ID NO: 1571. 78. The composition of embodiment 77, wherein the engineered guide RNA comprises SEQ ID NO: 1571. 79. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 49 relative to position 0, a 2/2 symmetric bulge at position 61 relative to position 0, a 2/2 symmetric bulge at position 73 relative to position 0, and any combination thereof. 80. The composition of embodiment 79, wherein the engineered guide RNA comprises at least about: 80%, 85%, 90%, 92%, 95%, 97%, or 99% sequence identity to SEQ ID NO: 1574. 81. The composition of embodiment 80, wherein the engineered guide RNA comprises SEQ ID NO: 1574. 82. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 47 relative to position 0, a 2/2 symmetric bulge at position 57 relative to position 0, a 2/2 symmetric bulge at position 67 relative to position 0, a 2/2 symmetric bulge at position 77 relative to position 0, and any combination thereof. 83. The composition of embodiment 82, wherein the engineered guide RNA comprises at least about: 80%, 85%, 90%, 92%, 95%, 97%, or 99% sequence identity to SEQ ID NO: 1570. 84. The composition of embodiment 83, wherein the engineered guide RNA comprises SEQ ID NO: 1570. 85. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 45 relative to position 0, a 2/2 symmetric bulge at position 53 relative to position 0, a 2/2 symmetric bulge at position 61 relative to position 0, a 2/2 symmetric bulge at position 69 relative to position 0, a 2/2 symmetric bulge at position 77 relative to position 0, and any combination thereof. 86. The composition of embodiment 85, wherein the engineered guide RNA comprises at least about: 80%, 85%, 90%, 92%, 95%, 97%, or 99% sequence identity to SEQ ID NO: 1566. 87. The composition of embodiment 86, wherein the engineered guide RNA comprises SEQ ID NO: 1566. 88. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 47 relative to position 0, a 4/4 symmetric bulge at position 59 relative to position 0, a 4/4 symmetric bulge at position 71 relative to position 0, and any combination thereof. 89. The composition of embodiment 88, wherein the engineered guide RNA comprises at least about: 80%, 85%, 90%, 92%, 95%, 97%, or 99% sequence identity to SEQ ID NO: 1572. 90. The composition of embodiment 89, wherein the engineered guide RNA comprises SEQ ID NO: 1572. 91. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 55 relative to position 0, a 3/3 symmetric bulge at position 74 relative to position 0, and any combination thereof 92. The composition of embodiment 91, wherein the engineered guide RNA comprises at least about: 80%, 85%, 90%, 92%, 95%, 97%, or 99% sequence identity to SEQ ID NO: 1587. 93. The composition of embodiment 92, wherein the engineered guide RNA comprises SEQ ID NO: 1587. 94. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof 95. The composition of embodiment 94, wherein the engineered guide RNA comprises at least about: 80%, 85%, 90%, 92%, 95%, 97%, or 99% sequence identity to SEQ ID NO: 1588. 96. The composition of embodiment 95, wherein the engineered guide RNA comprises SEQ ID NO: 1588. 97. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 55 relative to position 0, a 2/2 symmetric bulge at position 73 relative to position 0, and any combination thereof 98. The composition of embodiment 97, wherein the engineered guide RNA comprises at least about: 80%, 85%, 90%, 92%, 95%, 97%, or 99% sequence identity to SEQ ID NO: 1586. 99. The composition of embodiment 98, wherein the engineered guide RNA comprises SEQ ID NO: 1586. 100. The composition of embodiment 39, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 51 relative to position 0, a 4/4 symmetric bulge at position 67 relative to position 0, and any combination thereof. 101. The composition of embodiment 100, wherein the engineered guide RNA comprises at least about: 80%, 85%, 90%, 92%, 95%, 97%, or 99% sequence identity to SEQ ID NO: 1580. 102. The composition of embodiment 101, wherein the engineered guide RNA comprises SEQ ID NO: 1580. 103. The composition of embodiment 39, wherein the one or more structural features further comprises at least one structural feature selected from the group consisting of: an A/C mismatch at position 0, a second 6/6 symmetric internal loop at position 44 relative to position 0, and a combination thereof 104. The composition of embodiment 103, wherein the engineered guide RNA comprises at least about: 80%, 85%, 90%, 92%, 95%, 97%, or 99% sequence identity to SEQ ID NO: 985. 105. The composition of embodiment 104, wherein the engineered guide RNA comprises SEQ ID NO: 985. 106. The composition of embodiment 39, wherein the one or more structural features further comprises at least one structural feature selected from the group consisting of: an A/C mismatch at position 0, a second 6/6 symmetric internal loop at position 42 relative to position 0, and a combination thereof. 107. The composition of embodiment 106, wherein the engineered guide RNA comprises at least about: 80%, 85%, 90%, 92%, 95%, 97%, or 99% sequence identity to SEQ ID NO: 976. 108. The composition of embodiment 107, wherein the engineered guide RNA comprises SEQ ID NO: 976. 109. The composition of embodiment 11, wherein the first 6/6 symmetric internal loop is at position −7 relative to position 0. 110. The composition of embodiment 109, wherein the one or more structural features further comprises at least one structural feature selected from the group consisting of: an A/C mismatch at position 0, a second 6/6 symmetric internal loop at position 25 relative to position 0, and a combination thereof. 111. The composition of embodiment 110, wherein the engineered guide RNA comprises at least about: 80%, 85%, 90%, 92%, 95%, 97%, or 99% sequence identity to SEQ ID NO: 769. 112. The composition of embodiment 111, wherein the engineered guide RNA comprises SEQ ID NO: 769. 113. The composition of embodiment 109, wherein the one or more structural features further comprises at least one structural feature selected from the group consisting of: an A/C mismatch at position 0, a second 6/6 symmetric internal loop at position 42 relative to position 0, and a combination thereof 114. The composition of embodiment 113, wherein the engineered guide RNA comprises at least about: 80%, 85%, 90%, 92%, 95%, 97%, or 99% sequence identity to SEQ ID NO: 851. 115. The composition of embodiment 114, wherein the engineered guide RNA comprises SEQ ID NO: 851. 116. The composition of embodiment 11, wherein the first 6/6 symmetric internal loop is at position −8 relative to position 0. 117. The composition of embodiment 116, wherein the one or more structural features further comprises at least one structural feature selected from the group consisting of: an A/C mismatch at position 0, a second 6/6 symmetric internal loop at position 32 relative to position 0, and a combination thereof 118. The composition of embodiment 117, wherein the engineered guide RNA comprises at least about: 80%, 85%, 90%, 92%, 95%, 97%, or 99% sequence identity to SEQ ID NO: 679. 119. The composition of embodiment 118, wherein the engineered guide RNA comprises SEQ ID NO: 679. 120. The composition of embodiment 116, wherein the one or more structural features further comprises at least one structural feature selected from the group consisting of: an A/C mismatch at position 0, a second 6/6 symmetric internal loop at position 42 relative to position 0, and a combination thereof. 121. The composition of embodiment 120, wherein the engineered guide RNA comprises at least about: 80%, 85%, 90%, 92%, 95%, 97%, or 99% sequence identity to SEQ ID NO: 727. 122. The composition of embodiment 121, wherein the engineered guide RNA comprises SEQ ID NO: 727. 123. The composition of embodiment 116, wherein the one or more structural features further comprises at least one structural feature selected from the group consisting of: an A/C mismatch at position 0, a second 6/6 symmetric internal loop at position 24 relative to position 0, and a combination thereof. 124. The composition of embodiment 123, wherein the engineered guide RNA comprises at least about: 80%, 85%, 90%, 92%, 95%, 97%, or 99% sequence identity to SEQ ID NO: 642. 125. The composition of embodiment 124, wherein the engineered guide RNA comprises SEQ ID NO: 642. 126. The composition of embodiment 116, wherein the one or more structural features further comprises at least one structural feature selected from the group consisting of: an A/C mismatch at position 0, a second 6/6 symmetric internal loop at position 44 relative to position 0, and a combination thereof. 127. The composition of embodiment 126, wherein the engineered guide RNA comprises at least about: 80%, 85%, 90%, 92%, 95%, 97%, or 99% sequence identity to SEQ ID NO: 737. 128. The composition of embodiment 127, wherein the engineered guide RNA comprises SEQ ID NO: 737. 129. The composition of embodiment 11, wherein the first 6/6 symmetric internal loop is at position −9 relative to position 0. 130. The composition of embodiment 129, wherein the one or more structural features further comprises at least one structural feature selected from the group consisting of: an A/C mismatch at position 0, a second 6/6 symmetric internal loop at position 22 relative to position 0, and a combination thereof 131. The composition of embodiment 130, wherein the engineered guide RNA comprises at least about: 80%, 85%, 90%, 92%, 95%, 97%, or 99% sequence identity to SEQ ID NO: 512. 132. The composition of embodiment 131, wherein the engineered guide RNA comprises SEQ ID NO: 512. 133. The composition of embodiment 129, wherein the one or more structural features further comprises at least one structural feature selected from the group consisting of: an A/C mismatch at position 0, a second 6/6 symmetric internal loop at position 40 relative to position 0, and a combination thereof. 134. The composition of embodiment 133, wherein the engineered guide RNA comprises at least about: 80%, 85%, 90%, 92%, 95%, 97%, or 99% sequence identity to SEQ ID NO: 593. 135. The composition of embodiment 134, wherein the engineered guide RNA comprises SEQ ID NO: 593. 136. The composition of embodiment 129, wherein the one or more structural features further comprises at least one structural feature selected from the group consisting of: an A/C mismatch at position 0, a second 6/6 symmetric internal loop at position 20 relative to position 0, and a combination thereof. 137. The composition of embodiment 136, wherein the engineered guide RNA comprises at least about: 80%, 85%, 90%, 92%, 95%, 97%, or 99% sequence identity to SEQ ID NO: 502. 138. The composition of embodiment 137, wherein the engineered guide RNA comprises SEQ ID NO: 502. 139. The composition of embodiment 11, wherein the first 6/6 symmetric internal loop is at position −10 relative to position 0. 140. The composition of embodiment 139, wherein the one or more structural features further comprises at least one structural feature selected from the group consisting of: an A/C mismatch at position 0, a second 6/6 symmetric internal loop at position 43 relative to position 0, and a combination thereof. 141. The composition of embodiment 140, wherein the engineered guide RNA comprises at least about: 80%, 85%, 90%, 92%, 95%, 97%, or 99% sequence identity to SEQ ID NO: 487. 142. The composition of embodiment 141, wherein the engineered guide RNA comprises SEQ ID NO: 487. 143. The composition of embodiment 139, wherein the one or more structural features further comprises at least one structural feature selected from the group consisting of: an A/C mismatch at position 0, a second 6/6 symmetric internal loop at position 27 relative to position 0, and a combination thereof 144. The composition of embodiment 143, wherein the engineered guide RNA comprises at least about: 80%, 85%, 90%, 92%, 95%, 97%, or 99% sequence identity to SEQ ID NO: 408. 145. The composition of embodiment 144, wherein the engineered guide RNA comprises SEQ ID NO: 408. 146. The composition of embodiment 139, wherein the one or more structural features further comprises at least one structural feature selected from the group consisting of: an A/C mismatch at position 0, a second 6/6 symmetric internal loop at position 24 relative to position 0, and a combination thereof. 147. The composition of embodiment 146, wherein the engineered guide RNA comprises at least about: 80%, 85%, 90%, 92%, 95%, 97%, or 99% sequence identity to SEQ ID NO: 394. 148. The composition of embodiment 147, wherein the engineered guide RNA comprises SEQ ID NO: 394. 149. The composition of embodiment 139, wherein the one or more structural features further comprises at least one structural feature selected from the group consisting of: an A/C mismatch at position 0, a second 6/6 symmetric internal loop at position 42 relative to position 0, and a combination thereof. 150. The composition of embodiment 146, wherein the engineered guide RNA comprises at least about: 80%, 85%, 90%, 92%, 95%, 97%, or 99% sequence identity to SEQ ID NO: 482. 151. The composition of embodiment 147, wherein the engineered guide RNA comprises SEQ ID NO: 482. 152. The composition of embodiment 139, wherein the one or more structural features further comprises at least one structural feature selected from the group consisting of: an A/C mismatch at position 0, a second 6/6 symmetric internal loop at position 20 relative to position 0, and a combination thereof 153. The composition of embodiment 152, wherein the engineered guide RNA comprises at least about: 80%, 85%, 90%, 92%, 95%, 97%, or 99% sequence identity to SEQ ID NO: 375. 154. The composition of embodiment 153, wherein the engineered guide RNA comprises SEQ ID NO: 375. 155. The composition of embodiment 11, wherein the first 6/6 symmetric internal loop is at position −11 relative to position 0. 156. The composition of embodiment 155, wherein the one or more structural features further comprises at least one structural feature selected from the group consisting of: an A/C mismatch at position 0, a second 6/6 symmetric internal loop at position 44 relative to position 0, and a combination thereof 157. The composition of embodiment 156, wherein the engineered guide RNA comprises at least about: 80%, 85%, 90%, 92%, 95%, 97%, or 99% sequence identity to SEQ ID NO: 365. 158. The composition of embodiment 157, wherein the engineered guide RNA comprises SEQ ID NO: 365. 159. The composition of embodiment 155, wherein the one or more structural features further comprises at least one structural feature selected from the group consisting of: an A/C mismatch at position 0, a second 6/6 symmetric internal loop at position 42 relative to position 0, and a combination thereof. 160. The composition of embodiment 159, wherein the engineered guide RNA comprises at least about: 80%, 85%, 90%, 92%, 95%, 97%, or 99% sequence identity to SEQ ID NO: 356. 161. The composition of embodiment 160, wherein the engineered guide RNA comprises SEQ ID NO: 356. 162. The composition of embodiment 155, wherein the one or more structural features further comprises at least one structural feature selected from the group consisting of: an A/C mismatch at position 0, a second 6/6 symmetric internal loop at position 41 relative to position 0, and a combination thereof. 163. The composition of embodiment 162, wherein the engineered guide RNA comprises at least about: 80%, 85%, 90%, 92%, 95%, 97%, or 99% sequence identity to SEQ ID NO: 352. 164. The composition of embodiment 163, wherein the engineered guide RNA comprises SEQ ID NO: 352. 165. The composition of embodiment 155, wherein the one or more structural features further comprises at least one structural feature selected from the group consisting of: an A/C mismatch at position 0, a second 6/6 symmetric internal loop at position 20 relative to position 0, and a combination thereof 166. The composition of embodiment 165, wherein the engineered guide RNA comprises at least about: 80%, 85%, 90%, 92%, 95%, 97%, or 99% sequence identity to SEQ ID NO: 252. 167. The composition of embodiment 166, wherein the engineered guide RNA comprises SEQ ID NO: 252. 168. The composition of embodiment 155, wherein the one or more structural features further comprises at least one structural feature selected from the group consisting of: an A/C mismatch at position 0, a second 6/6 symmetric internal loop at position 28 relative to position 0, and a combination thereof. 169. The composition of embodiment 168, wherein the engineered guide RNA comprises at least about: 80%, 85%, 90%, 92%, 95%, 97%, or 99% sequence identity to SEQ ID NO: 291. 170. The composition of embodiment 169, wherein the engineered guide RNA comprises SEQ ID NO: 291. 171. The composition of any one of embodiments 6-9, wherein position 3 of ATTAAA is the target adenosine, wherein position 3 is the second A of ATTAAA from the 5′ end. 172. The composition of embodiment 171, wherein the one or more structural features comprises: a first 6/6 symmetric internal loop at a position selected from the group consisting of: 22, 21, 20, −2, −4, −5, −6, −7, −8, −9, and −10 relative to position 0 of ATTAAA. 173. The composition of embodiment 172, wherein the first 6/6 symmetric internal loop is at position 22 relative to position 0. 174. The composition of embodiment 173, wherein the one or more structural features further comprises an A/C mismatch at position 3 relative to position 0. 175. The composition of embodiment 174, wherein the engineered guide RNA comprises at least about: 80%, 85%, 90%, 92%, 95%, 97%, or 99% sequence identity to SEQ ID NO: 17. 176. The composition of embodiment 175, wherein the engineered guide RNA comprises SEQ ID NO: 17. 177. The composition of embodiment 172, wherein the first 6/6 symmetric internal loop is at position 21 relative to position 0. 178. The composition of embodiment 177, wherein the engineered guide RNA comprises at least about: 80%, 85%, 90%, 92%, 95%, 97%, or 99% sequence identity to SEQ ID NO: 15. 179. The composition of embodiment 178, wherein the engineered guide RNA comprises SEQ ID NO: 15. 180. The composition of embodiment 177, wherein the one or more structural features further comprises an A/C mismatch at position 5 relative to position 0. 181. The composition of embodiment 180, wherein the engineered guide RNA comprises at least about: 80%, 85%, 90%, 92%, 95%, 97%, or 99% sequence identity to SEQ ID NO: 14. 182. The composition of embodiment 181, wherein the engineered guide RNA comprises SEQ ID NO: 14. 183. The composition of embodiment 172, wherein the first 6/6 symmetric internal loop is at position 20 relative to position 0. 184. The composition of embodiment 183, wherein the one or more structural features further comprises an A/C mismatch at position 5 relative to position 0. 185. The composition of embodiment 184, wherein the engineered guide RNA comprises at least about: 80%, 85%, 90%, 92%, 95%, 97%, or 99% sequence identity to SEQ ID NO: 10. 186. The composition of embodiment 185, wherein the engineered guide RNA comprises SEQ ID NO: 10. 187. The composition of embodiment 183, wherein the one or more structural features further comprises an A/C mismatch at position 3 relative to position 0. 188. The composition of embodiment 187, wherein the engineered guide RNA comprises at least about: 80%, 85%, 90%, 92%, 95%, 97%, or 99% sequence identity to SEQ ID NO: 8. 189. The composition of embodiment 188, wherein the engineered guide RNA comprises SEQ ID NO: 8. 190. The composition of embodiment 172, wherein the first 6/6 symmetric internal loop is at position −2 relative to position 0. 191. The composition of embodiment 190, wherein the one or more structural features further comprises at least one structural feature selected from the group consisting of: an A/C mismatch at position 5 relative to position 0, a second 6/6 symmetric internal loop at position 42 relative to position 0, and a combination thereof. 192. The composition of embodiment 191, wherein the engineered guide RNA comprises at least about: 80%, 85%, 90%, 92%, 95%, 97%, or 99% sequence identity to SEQ ID NO: 1411. 193. The composition of embodiment 192, wherein the engineered guide RNA comprises SEQ ID NO: 1411. 194. The composition of embodiment 172, wherein the first 6/6 symmetric internal loop is at position −4 relative to position 0. 195. The composition of embodiment 194, wherein the one or more structural features further comprises a second 6/6 symmetric internal loop at position 22 relative to position 0. 196. The composition of embodiment 195, wherein the engineered guide RNA comprises at least about: 80%, 85%, 90%, 92%, 95%, 97%, or 99% sequence identity to SEQ ID NO: 1116. 197. The composition of embodiment 196, wherein the engineered guide RNA comprises SEQ ID NO: 1116. 198. The composition of embodiment 172, wherein the first 6/6 symmetric internal loop is at position −5 relative to position 0. 199. The composition of embodiment 198, wherein the one or more structural features further comprises at least one structural feature selected from the group consisting of: an A/C mismatch at position 5 relative to position 0, a second 6/6 symmetric internal loop at position 22 relative to position 0, and a combination thereof. 200. The composition of embodiment 199, wherein the engineered guide RNA comprises at least about: 80%, 85%, 90%, 92%, 95%, 97%, or 99% sequence identity to SEQ ID NO: 1002. 201. The composition of embodiment 200, wherein the engineered guide RNA comprises SEQ ID NO: 1002. 202. The composition of embodiment 198, wherein the one or more structural features further comprises a second 6/6 symmetric internal loop at position 33 relative to position 0. 203. The composition of embodiment 202, wherein the engineered guide RNA comprises at least about: 80%, 85%, 90%, 92%, 95%, 97%, or 99% sequence identity to SEQ ID NO: 1054. 204. The composition of embodiment 203, wherein the engineered guide RNA comprises SEQ ID NO: 1054. 205. The composition of embodiment 172, wherein the first 6/6 symmetric internal loop is at position −6 relative to position 0. 206. The composition of embodiment 205, wherein the one or more structural features further comprises at least one structural feature selected from the group consisting of: an A/C mismatch at position 3 relative to position 0, a second 6/6 symmetric internal loop at position 24 relative to position 0, and a combination thereof. 207. The composition of embodiment 206, wherein the engineered guide RNA comprises at least about: 80%, 85%, 90%, 92%, 95%, 97%, or 99% sequence identity to SEQ ID NO: 892. 208. The composition of embodiment 207, wherein the engineered guide RNA comprises SEQ ID NO: 892. 209. The composition of embodiment 205, wherein the one or more structural features further comprises a second 6/6 symmetric internal loop at position 21 relative to position 0. 210. The composition of embodiment 209, wherein the engineered guide RNA comprises at least about: 80%, 85%, 90%, 92%, 95%, 97%, or 99% sequence identity to SEQ ID NO: 880. 211. The composition of embodiment 210, wherein the engineered guide RNA comprises SEQ ID NO: 880. 212. The composition of embodiment 205, wherein the one or more structural features further comprises at least one structural feature selected from the group consisting of: an A/C mismatch at position 3 relative to position 0, a second 6/6 symmetric internal loop at position 42 relative to position 0, and a combination thereof 213. The composition of embodiment 212, wherein the engineered guide RNA comprises at least about: 80%, 85%, 90%, 92%, 95%, 97%, or 99% sequence identity to SEQ ID NO: 977. 214. The composition of embodiment 213, wherein the engineered guide RNA comprises SEQ ID NO: 977. 215. The composition of embodiment 205, wherein the one or more structural features further comprises at least one structural feature selected from the group consisting of: an A/C mismatch at position 5 relative to position 0, a second 6/6 symmetric internal loop at position 20 relative to position 0, and a combination thereof 216. The composition of embodiment 215, wherein the engineered guide RNA comprises at least about: 80%, 85%, 90%, 92%, 95%, 97%, or 99% sequence identity to SEQ ID NO: 874. 217. The composition of embodiment 216, wherein the engineered guide RNA comprises SEQ ID NO: 874. 218. The composition of embodiment 205, wherein the one or more structural features further comprises at least one structural feature selected from the group consisting of: an A/C mismatch at position 4 relative to position 0, a second 6/6 symmetric internal loop at position 20 relative to position 0, and a combination thereof 219. The composition of embodiment 218, wherein the engineered guide RNA comprises at least about: 80%, 85%, 90%, 92%, 95%, 97%, or 99% sequence identity to SEQ ID NO: 873. 220. The composition of embodiment 219, wherein the engineered guide RNA comprises SEQ ID NO: 873. 221. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof. 222. The composition of embodiment 221, wherein the engineered guide RNA comprises at least about: 80%, 85%, 90%, 92%, 95%, 97%, or 99% sequence identity to SEQ ID NO: 1569. 223. The composition of embodiment 222, wherein the engineered guide RNA comprises SEQ ID NO: 1569. 224. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof. 225. The composition of embodiment 224, wherein the engineered guide RNA comprises at least about: 80%, 85%, 90%, 92%, 95%, 97%, or 99% sequence identity to SEQ ID NO: 1567. 226. The composition of embodiment 225, wherein the engineered guide RNA comprises SEQ ID NO: 1567. 227. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 55 relative to position 0, a 3/3 symmetric bulge at position 74 relative to position 0, and any combination thereof. 228. The composition of embodiment 227, wherein the engineered guide RNA comprises at least about: 80%, 85%, 90%, 92%, 95%, 97%, or 99% sequence identity to SEQ ID NO: 1587. 229. The composition of embodiment 228, wherein the engineered guide RNA comprises SEQ ID NO: 1587. 230. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 47 relative to position 0, a 3/3 symmetric bulge at position 58 relative to position 0, a 3/3 symmetric bulge at position 69 relative to position 0, and any combination thereof. 231. The composition of embodiment 230, wherein the engineered guide RNA comprises at least about: 80%, 85%, 90%, 92%, 95%, 97%, or 99% sequence identity to SEQ ID NO: 1571. 232. The composition of embodiment 231, wherein the engineered guide RNA comprises SEQ ID NO: 1571. 233. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 53 relative to position 0, a 5/5 symmetric internal loop at position 72 relative to position 0, and any combination thereof. 234. The composition of embodiment 233, wherein the engineered guide RNA comprises at least about: 80%, 85%, 90%, 92%, 95%, 97%, or 99% sequence identity to SEQ ID NO: 1585. 235. The composition of embodiment 234, wherein the engineered guide RNA comprises SEQ ID NO: 1585. 236. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof. 237. The composition of embodiment 236, wherein the engineered guide RNA comprises at least about: 80%, 85%, 90%, 92%, 95%, 97%, or 99% sequence identity to SEQ ID NO: 1573. 238. The composition of embodiment 237, wherein the engineered guide RNA comprises SEQ ID NO: 1573. 239. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof. 240. The composition of embodiment 239, wherein the engineered guide RNA comprises at least about: 80%, 85%, 90%, 92%, 95%, 97%, or 99% sequence identity to SEQ ID NO: 1588. 241. The composition of embodiment 240, wherein the engineered guide RNA comprises SEQ ID NO: 1588. 242. The composition of embodiment 205, wherein the one or more structural features further comprises at least one structural feature selected from the group consisting of: A/C mismatch at position 3, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof. 243. The composition of embodiment 242, wherein the engineered guide RNA comprises at least about: 80%, 85%, 90%, 92%, 95%, 97%, or 99% sequence identity to SEQ ID NO: 1575. 244. The composition of embodiment 243, wherein the engineered guide RNA comprises SEQ ID NO: 1575. 245. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 53 relative to position 0, a 4/4 symmetric bulge at position 71 relative to position 0, and any combination thereof. 246. The composition of embodiment 245, wherein the engineered guide RNA comprises at least about: 80%, 85%, 90%, 92%, 95%, 97%, or 99% sequence identity to SEQ ID NO: 1584. 247. The composition of embodiment 246, wherein the engineered guide RNA comprises SEQ ID NO: 1584. 248. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 47 relative to position 0, a 4/4 symmetric bulge at position 59 relative to position 0, a 4/4 symmetric bulge at position 71 relative to position 0, and any combination thereof. 249. The composition of embodiment 248, wherein the engineered guide RNA comprises at least about: 80%, 85%, 90%, 92%, 95%, 97%, or 99% sequence identity to SEQ ID NO: 1572. 250. The composition of embodiment 249, wherein the engineered guide RNA comprises SEQ ID NO: 1572. 251. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 47 relative to position 0, a 2/2 symmetric bulge at position 57 relative to position 0, a 2/2 symmetric bulge at position 67 relative to position 0, a 2/2 symmetric bulge at position 77 relative to position 0, and any combination thereof. 252. The composition of embodiment 251, wherein the engineered guide RNA comprises at least about: 80%, 85%, 90%, 92%, 95%, 97%, or 99% sequence identity to SEQ ID NO: 1570. 253. The composition of embodiment 252, wherein the engineered guide RNA comprises SEQ ID NO: 1570. 254. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 49 relative to position 0, a 2/2 symmetric bulge at position 61 relative to position 0, a 2/2 symmetric bulge at position 73 relative to position 0, and any combination thereof. 255. The composition of embodiment 254, wherein the engineered guide RNA comprises at least about: 80%, 85%, 90%, 92%, 95%, 97%, or 99% sequence identity to SEQ ID NO: 1574. 256. The composition of embodiment 255, wherein the engineered guide RNA comprises SEQ ID NO: 1574. 257. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 51 relative to position 0, a 5/5 symmetric internal loop at position 68 relative to position 0, and any combination thereof. 258. The composition of embodiment 257, wherein the engineered guide RNA comprises at least about: 80%, 85%, 90%, 92%, 95%, 97%, or 99% sequence identity to SEQ ID NO: 1581. 259. The composition of embodiment 258, wherein the engineered guide RNA comprises SEQ ID NO: 1581. 260. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 45 relative to position 0, a 2/2 symmetric bulge at position 53 relative to position 0, a 2/2 symmetric bulge at position 61 relative to position 0, a 2/2 symmetric bulge at position 69 relative to position 0, a 2/2 symmetric bulge at position 77 relative to position 0, and any combination thereof. 261. The composition of embodiment 260, wherein the engineered guide RNA comprises at least about: 80%, 85%, 90%, 92%, 95%, 97%, or 99% sequence identity to SEQ ID NO: 1566. 262. The composition of embodiment 261, wherein the engineered guide RNA comprises SEQ ID NO: 1566. 263. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 45 relative to position 0, a 4/4 symmetric bulge at position 55 relative to position 0, a 4/4 symmetric bulge at position 65 relative to position 0, a 4/4 symmetric bulge at position 75 relative to position 0, and any combination thereof. 264. The composition of embodiment 263, wherein the engineered guide RNA comprises at least about: 80%, 85%, 90%, 92%, 95%, 97%, or 99% sequence identity to SEQ ID NO: 1568. 265. The composition of embodiment 264, wherein the engineered guide RNA comprises SEQ ID NO: 1568. 266. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 51 relative to position 0, a 4/4 symmetric bulge at position 67 relative to position 0, and any combination thereof. 267. The composition of embodiment 266, wherein the engineered guide RNA comprises at least about: 80%, 85%, 90%, 92%, 95%, 97%, or 99% sequence identity to SEQ ID NO: 1580. 268. The composition of embodiment 267, wherein the engineered guide RNA comprises SEQ ID NO: 1580. 269. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 55 relative to position 0, a 2/2 symmetric bulge at position 73 relative to position 0, and any combination thereof. 270. The composition of embodiment 269, wherein the engineered guide RNA comprises at least about: 80%, 85%, 90%, 92%, 95%, 97%, or 99% sequence identity to SEQ ID NO: 1586. 271. The composition of embodiment 270, wherein the engineered guide RNA comprises SEQ ID NO: 1586. 272. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 51 relative to position 0, a 2/2 symmetric bulge at position 65 relative to position 0, and any combination thereof. 273. The composition of embodiment 272, wherein the engineered guide RNA comprises at least about: 80%, 85%, 90%, 92%, 95%, 97%, or 99% sequence identity to SEQ ID NO: 1578. 274. The composition of embodiment 273, wherein the engineered guide RNA comprises SEQ ID NO: 1578. 275. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 51 relative to position 0, a 3/3 symmetric bulge at position 66 relative to position 0, and any combination thereof. 276. The composition of embodiment 275, wherein the engineered guide RNA comprises at least about: 80%, 85%, 90%, 92%, 95%, 97%, or 99% sequence identity to SEQ ID NO: 1579. 277. The composition of embodiment 276, wherein the engineered guide RNA comprises SEQ ID NO: 1579. 278. The composition of embodiment 205, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 49 relative to position 0, a 5/5 symmetric internal loop at position 64 relative to position 0, and any combination thereof. 279. The composition of embodiment 278, wherein the engineered guide RNA comprises at least about: 80%, 85%, 90%, 92%, 95%, 97%, or 99% sequence identity to SEQ ID NO: 1577. 280. The composition of embodiment 279, wherein the engineered guide RNA comprises SEQ ID NO: 1577. 281. The composition of embodiment 205, wherein the one or more structural features further comprises at least one structural feature selected from the group consisting of: an A/C mismatch at position 5 relative to position 0, a second 6/6 symmetric internal loop at position 22 relative to position 0, and a combination thereof. 282. The composition of embodiment 281, wherein the engineered guide RNA comprises at least about: 80%, 85%, 90%, 92%, 95%, 97%, or 99% sequence identity to SEQ ID NO: 884. 283. The composition of embodiment 282, wherein the engineered guide RNA comprises SEQ ID NO: 884. 284. The composition of embodiment 205, wherein the one or more structural features further comprises at least one structural feature selected from the group consisting of: an A/C mismatch at position 0, a second 6/6 symmetric internal loop at position 20 relative to position 0, and a combination thereof. 285. The composition of embodiment 284, wherein the engineered guide RNA comprises at least about: 80%, 85%, 90%, 92%, 95%, 97%, or 99% sequence identity to SEQ ID NO: 871. 286. The composition of embodiment 285, wherein the engineered guide RNA comprises SEQ ID NO: 871. 287. The composition of embodiment 172, wherein the first 6/6 symmetric internal loop is at position −7 relative to position 0. 288. The composition of embodiment 287, wherein the one or more structural features further comprises at least one structural feature selected from the group consisting of: an A/C mismatch at position 3 relative to position 0, a second 6/6 symmetric internal loop at position 20 relative to position 0, and a combination thereof 289. The composition of embodiment 288, wherein the engineered guide RNA comprises at least about: 80%, 85%, 90%, 92%, 95%, 97%, or 99% sequence identity to SEQ ID NO: 747. 290. The composition of embodiment 289, wherein the engineered guide RNA comprises SEQ ID NO: 747. 291. The composition of embodiment 287, wherein the one or more structural features further comprises at least one structural feature selected from the group consisting of: an A/C mismatch at position 5 relative to position 0, a second 6/6 symmetric internal loop at position 22 relative to position 0, and a combination thereof. 292. The composition of embodiment 291, wherein the engineered guide RNA comprises at least about: 80%, 85%, 90%, 92%, 95%, 97%, or 99% sequence identity to SEQ ID NO: 757. 293. The composition of embodiment 292, wherein the engineered guide RNA comprises SEQ ID NO: 757. 294. The composition of embodiment 287, wherein the one or more structural features further comprises at least one structural feature selected from the group consisting of: an A/C mismatch at position 5 relative to position 0, a second 6/6 symmetric internal loop at position 20 relative to position 0, and a combination thereof. 295. The composition of embodiment 294, wherein the engineered guide RNA comprises at least about: 80%, 85%, 90%, 92%, 95%, 97%, or 99% sequence identity to SEQ ID NO: 748. 296. The composition of embodiment 295, wherein the engineered guide RNA comprises SEQ ID NO: 748. 297. The composition of embodiment 172, wherein the first 6/6 symmetric internal loop is at position −8 relative to position 0. 298. The composition of embodiment 297, wherein the one or more structural features further comprises at least one structural feature selected from the group consisting of: A/C mismatch at position 5, a second 6/6 symmetric internal loop at position 20 relative to position 0, and a combination thereof. 299. The composition of embodiment 298, wherein the engineered guide RNA comprises at least about: 80%, 85%, 90%, 92%, 95%, 97%, or 99% sequence identity to SEQ ID NO: 625. 300. The composition of embodiment 299, wherein the engineered guide RNA comprises SEQ ID NO: 625. 301. The composition of embodiment 297, wherein the one or more structural features further comprises at least one structural feature selected from the group consisting of: an A/C mismatch at position 5 relative to position 0, a second 6/6 symmetric internal loop at position 22 relative to position 0, and a combination thereof. 302. The composition of embodiment 301, wherein the engineered guide RNA comprises at least about: 80%, 85%, 90%, 92%, 95%, 97%, or 99% sequence identity to SEQ ID NO: 635. 303. The composition of embodiment 302, wherein the engineered guide RNA comprises SEQ ID NO: 635. 304. The composition of embodiment 172, wherein the first 6/6 symmetric internal loop is at position −9 relative to position 0. 305. The composition of embodiment 304, wherein the one or more structural features further comprises at least one structural feature selected from the group consisting of: an A/C mismatch at position 5 relative to position 0, a second 6/6 symmetric internal loop at position 20 relative to position 0, and a combination thereof. 306. The composition of embodiment 305, wherein the engineered guide RNA comprises at least about: 80%, 85%, 90%, 92%, 95%, 97%, or 99% sequence identity to SEQ ID NO: 505. 307. The composition of embodiment 306, wherein the engineered guide RNA comprises SEQ ID NO: 505. 308. The composition of embodiment 304, wherein the one or more structural features further comprises a second 6/6 symmetric internal loop at position 42 relative to position 0. 309. The composition of embodiment 308, wherein the engineered guide RNA comprises at least about: 80%, 85%, 90%, 92%, 95%, 97%, or 99% sequence identity to SEQ ID NO: 606. 310. The composition of embodiment 309, wherein the engineered guide RNA comprises SEQ ID NO: 606. 311. The composition of embodiment 304, wherein the one or more structural features further comprises at least one structural feature selected from the group consisting of: an A/C mismatch at position 0, a second 6/6 symmetric internal loop at position 40 relative to position 0, and a combination thereof. 312. The composition of embodiment 311, wherein the engineered guide RNA comprises at least about: 80%, 85%, 90%, 92%, 95%, 97%, or 99% sequence identity to SEQ ID NO: 593. 313. The composition of embodiment 312, wherein the engineered guide RNA comprises SEQ ID NO: 593. 314. The composition of embodiment 172, wherein the first 6/6 symmetric internal loop is at position −10 relative to position 0. 315. The composition of embodiment 314, wherein the one or more structural features further comprises a second 6/6 symmetric internal loop at position 42 relative to position 0. 316. The composition of embodiment 315, wherein the engineered guide RNA comprises at least about: 80%, 85%, 90%, 92%, 95%, 97%, or 99% sequence identity to SEQ ID NO: 486. 317. The composition of embodiment 316, wherein the engineered guide RNA comprises SEQ ID NO: 486. 318. The composition of embodiment 314, wherein the one or more structural features further comprises at least one structural feature selected from the group consisting of: an A/C mismatch at position 4 relative to position 0, a second 6/6 symmetric internal loop at position 44 relative to position 0, and a combination thereof. 319. The composition of embodiment 318, wherein the engineered guide RNA comprises at least about: 80%, 85%, 90%, 92%, 95%, 97%, or 99% sequence identity to SEQ ID NO: 494. 320. The composition of embodiment 319, wherein the engineered guide RNA comprises SEQ ID NO: 494. 321. The composition of embodiment 171, wherein the one or more structural features comprises: a first 2/2 symmetric bulge at a position selected from the group consisting of: −3, −5, and −7 relative to position 0 of ATTAAA. 322. The composition of embodiment 321, wherein the first 2/2 symmetric bulge is at position −3 relative to position 0. 323. The composition of embodiment 322, wherein the one or more structural features further comprises at least one structural feature selected from the group consisting of: a 2/2 symmetric bulge at position 14 relative to position 0, a 2/2 symmetric bulge at position 32 relative to position 0, a 2/2 symmetric bulge at position 50 relative to position 0, a 2/2 symmetric bulge at position 68 relative to position 0, and any combination thereof. 324. The composition of embodiment 323, wherein the engineered guide RNA comprises at least about: 80%, 85%, 90%, 92%, 95%, 97%, or 99% sequence identity to SEQ ID NO: 1552. 325. The composition of embodiment 324, wherein the engineered guide RNA comprises SEQ ID NO: 1552. 326. The composition of embodiment 321, wherein the first 2/2 symmetric bulge is at position −5 relative to position 0. 327. The composition of embodiment 326, wherein the one or more structural features further comprises at least one structural feature selected from the group consisting of: a 2/2 symmetric bulge at position 26 relative to position 0, a 2/2 symmetric bulge at position 42 relative to position 0, a 2/2 symmetric bulge at position 58 relative to position 0, a 2/2 symmetric bulge at position 74 relative to position 0, and any combination thereof. 328. The composition of embodiment 327, wherein the engineered guide RNA comprises at least about: 80%, 85%, 90%, 92%, 95%, 97%, or 99% sequence identity to SEQ ID NO: 1545. 329. The composition of embodiment 328, wherein the engineered guide RNA comprises SEQ ID NO: 1545. 330. The composition of embodiment 321, wherein the first 2/2 symmetric bulge is at position −7 relative to position 0. 331. The composition of embodiment 330, wherein the one or more structural features further comprises at least one structural feature selected from the group consisting of: a 2/2 symmetric bulge at position 6 relative to position 0, a 2/2 symmetric bulge at position 20 relative to position 0, a 2/2 symmetric bulge at position 34 relative to position 0, a 2/2 symmetric bulge at position 48 relative to position 0, a 2/2 symmetric bulge at position 62 relative to position 0, a 2/2 symmetric bulge at position 76 relative to position 0, and any combination thereof. 332. The composition of embodiment 331, wherein the engineered guide RNA comprises at least about: 80%, 85%, 90%, 92%, 95%, 97%, or 99% sequence identity to SEQ ID NO: 1538. 333. The composition of embodiment 332, wherein the engineered guide RNA comprises SEQ ID NO: 1538. 334. The composition of embodiment 171, wherein the one or more structural features comprises: a first 3/3 symmetric bulge at position −6 relative to position 0 of ATTAAA. 335. The composition of embodiment 334, wherein the one or more structural features further comprises at least one structural feature selected from the group consisting of: a 3/3 symmetric bulge at position 7 relative to position 0, a 3/3 symmetric bulge at position 22 relative to position 0, a 3/3 symmetric bulge at position 37 relative to position 0, a 3/3 symmetric bulge at position 52 relative to position 0, a 3/3 symmetric bulge at position 67 relative to position 0, and any combination thereof. 336. The composition of embodiment 335, wherein the engineered guide RNA comprises at least about: 80%, 85%, 90%, 92%, 95%, 97%, or 99% sequence identity to SEQ ID NO: 1539. 337. The composition of embodiment 336, wherein the engineered guide RNA comprises SEQ ID NO: 1539. 338. The composition of any one of embodiments 6-9, wherein position 4 of ATTAAA is the target adenosine, wherein position 4 is the third A of ATTAAA from the 5′ end. 339. The composition of embodiment 338, wherein the one or more structural features comprises: a first 6/6 symmetric internal loop at a position selected from the group consisting of: 33, −1, −2, −3, −4, −5, −6, −7, −8, −9, −11, and −12 relative to position 0 of ATTAAA. 340. The composition of embodiment 339, wherein the first 6/6 symmetric internal loop is at position 33 relative to position 0. 341. The composition of embodiment 340, wherein the one or more structural features further comprises an A/C mismatch at position 3 relative to position 0. 342. The composition of embodiment 341, wherein the engineered guide RNA comprises at least about: 80%, 85%, 90%, 92%, 95%, 97%, or 99% sequence identity to SEQ ID NO: 72. 343. The composition of embodiment 342, wherein the engineered guide RNA comprises SEQ ID NO: 72. 344. The composition of embodiment 339, wherein the first 6/6 symmetric internal loop is at position −1 relative to position 0. 345. The composition of embodiment 344, wherein the one or more structural features further comprises at least one structural feature selected from the group consisting of: an A/C mismatch at position 4 relative to position 0, a second 6/6 symmetric internal loop at position 32 relative to position 0, and a combination thereof 346. The composition of embodiment 345, wherein the engineered guide RNA comprises at least about: 80%, 85%, 90%, 92%, 95%, 97%, or 99% sequence identity to SEQ ID NO: 1463. 347. The composition of embodiment 346, wherein the engineered guide RNA comprises SEQ ID NO: 1463. 348. The composition of embodiment 339, wherein the first 6/6 symmetric internal loop is at position −2 relative to position 0. 349. The composition of embodiment 348, wherein the one or more structural features further comprises a second 6/6 symmetric internal loop at position 32 relative to position 0. 350. The composition of embodiment 349, wherein the engineered guide RNA comprises at least about: 80%, 85%, 90%, 92%, 95%, 97%, or 99% sequence identity to SEQ ID NO: 1374. 351. The composition of embodiment 350, wherein the engineered guide RNA comprises SEQ ID NO: 1374. 352. The composition of embodiment 348, wherein the one or more structural features further comprises at least one structural feature selected from the group consisting of: an A/C mismatch at position 3 relative to position 0, a second 6/6 symmetric internal loop at position 37 relative to position 0, and a combination thereof 353. The composition of embodiment 352, wherein the engineered guide RNA comprises at least about: 80%, 85%, 90%, 92%, 95%, 97%, or 99% sequence identity to SEQ ID NO: 1391. 354. The composition of embodiment 353, wherein the engineered guide RNA comprises SEQ ID NO: 1391. 355. The composition of embodiment 339, wherein the first 6/6 symmetric internal loop is at position −3 relative to position 0. 356. The composition of embodiment 355, wherein the one or more structural features further comprises at least one structural feature selected from the group consisting of: an A/C mismatch at position 3 relative to position 0, a second 6/6 symmetric internal loop at position 36 relative to position 0, and a combination thereof. 357. The composition of embodiment 356, wherein the engineered guide RNA comprises at least about: 80%, 85%, 90%, 92%, 95%, 97%, or 99% sequence identity to SEQ ID NO: 1293. 358. The composition of embodiment 357, wherein the engineered guide RNA comprises SEQ ID NO: 1293. 359. The composition of embodiment 355, wherein the one or more structural features further comprises at least one structural feature selected from the group consisting of: an A/C mismatch at position 4 relative to position 0, a second 6/6 symmetric internal loop at position 36 relative to position 0, and a combination thereof. 360. The composition of embodiment 359, wherein the engineered guide RNA comprises at least about: 80%, 85%, 90%, 92%, 95%, 97%, or 99% sequence identity to SEQ ID NO: 1294. 361. The composition of embodiment 360, wherein the engineered guide RNA comprises SEQ ID NO: 1294. 362. The composition of embodiment 355, wherein the one or more structural features further comprises a second 6/6 symmetric internal loop at position 36 relative to position 0. 363. The composition of embodiment 362, wherein the engineered guide RNA comprises at least about: 80%, 85%, 90%, 92%, 95%, 97%, or 99% sequence identity to SEQ ID NO: 1296. 364. The composition of embodiment 363, wherein the engineered guide RNA comprises SEQ ID NO: 1296. 365. The composition of embodiment 339, wherein the first 6/6 symmetric internal loop is at position −4 relative to position 0. 366. The composition of embodiment 365, wherein the one or more structural features further comprises at least one structural feature selected from the group consisting of: an A/C mismatch at position 3 relative to position 0, a second 6/6 symmetric internal loop at position 36 relative to position 0, and a combination thereof 367. The composition of embodiment 366, wherein the engineered guide RNA comprises at least about: 80%, 85%, 90%, 92%, 95%, 97%, or 99% sequence identity to SEQ ID NO: 1183. 368. The composition of embodiment 367, wherein the engineered guide RNA comprises SEQ ID NO: 1183. 369. The composition of embodiment 365, wherein the one or more structural features further comprises at least one structural feature selected from the group consisting of: an A/C mismatch at position 4 relative to position 0, a second 6/6 symmetric internal loop at position 38 relative to position 0, and a combination thereof 370. The composition of embodiment 369, wherein the engineered guide RNA comprises at least about: 80%, 85%, 90%, 92%, 95%, 97%, or 99% sequence identity to SEQ ID NO: 1193. 371. The composition of embodiment 370, wherein the engineered guide RNA comprises SEQ ID NO: 1193. 372. The composition of embodiment 365, wherein the one or more structural features further comprises at least one structural feature selected from the group consisting of: an A/C mismatch at position 3 relative to position 0, a second 6/6 symmetric internal loop at position 42 relative to position 0, and a combination thereof 373. The composition of embodiment 372, wherein the engineered guide RNA comprises at least about: 80%, 85%, 90%, 92%, 95%, 97%, or 99% sequence identity to SEQ ID NO: 1212. 374. The composition of embodiment 373, wherein the engineered guide RNA comprises SEQ ID NO: 1212. 375. The composition of embodiment 365, wherein the one or more structural features further comprises at least one structural feature selected from the group consisting of: an A/C mismatch at position 3 relative to position 0, a second 6/6 symmetric internal loop at position 33 relative to position 0, and a combination thereof 376. The composition of embodiment 375, wherein the engineered guide RNA comprises at least about: 80%, 85%, 90%, 92%, 95%, 97%, or 99% sequence identity to SEQ ID NO: 1168. 377. The composition of embodiment 376, wherein the engineered guide RNA comprises SEQ ID NO: 1168. 378. The composition of embodiment 339, wherein the first 6/6 symmetric internal loop is at position −5 relative to position 0. 379. The composition of embodiment 378, wherein the one or more structural features further comprises at least one structural feature selected from the group consisting of: an A/C mismatch at position 3 relative to position 0, a second 6/6 symmetric internal loop at position 36 relative to position 0, and a combination thereof. 380. The composition of embodiment 379, wherein the engineered guide RNA comprises at least about: 80%, 85%, 90%, 92%, 95%, 97%, or 99% sequence identity to SEQ ID NO: 1066. 381. The composition of embodiment 380, wherein the engineered guide RNA comprises SEQ ID NO: 1066. 382. The composition of embodiment 378, wherein the one or more structural features further comprises at least one structural feature selected from the group consisting of: an A/C mismatch at position 3 relative to position 0, a second 6/6 symmetric internal loop at position 33 relative to position 0, and a combination thereof. 383. The composition of embodiment 382, wherein the engineered guide RNA comprises at least about: 80%, 85%, 90%, 92%, 95%, 97%, or 99% sequence identity to SEQ ID NO: 1051. 384. The composition of embodiment 383, wherein the engineered guide RNA comprises SEQ ID NO: 1051. 385. The composition of embodiment 378, wherein the one or more structural features further comprises a second 6/6 symmetric internal loop at position 34 relative to position 0. 386. The composition of embodiment 385, wherein the engineered guide RNA comprises at least about: 80%, 85%, 90%, 92%, 95%, 97%, or 99% sequence identity to SEQ ID NO: 1059. 387. The composition of embodiment 386, wherein the engineered guide RNA comprises SEQ ID NO: 1059. 388. The composition of embodiment 378, wherein the one or more structural features further comprises a second 6/6 symmetric internal loop at position 33 relative to position 0. 389. The composition of embodiment 388, wherein the engineered guide RNA comprises at least about: 80%, 85%, 90%, 92%, 95%, 97%, or 99% sequence identity to SEQ ID NO: 1054. 390. The composition of embodiment 389, wherein the engineered guide RNA comprises SEQ ID NO: 1054. 391. The composition of embodiment 339, wherein the first 6/6 symmetric internal loop is at position −6 relative to position 0. 392. The composition of embodiment 391, wherein the one or more structural features further comprises at least one structural feature selected from the group consisting of: an A/C mismatch at position 0, a second 6/6 symmetric internal loop at position 40 relative to position 0, and a combination thereof. 393. The composition of embodiment 392, wherein the engineered guide RNA comprises at least about: 80%, 85%, 90%, 92%, 95%, 97%, or 99% sequence identity to SEQ ID NO: 967. 394. The composition of embodiment 393, wherein the engineered guide RNA comprises SEQ ID NO: 967. 395. The composition of embodiment 391, wherein the one or more structural features further comprises at least one structural feature selected from the group consisting of: an A/C mismatch at position 3 relative to position 0, a second 6/6 symmetric internal loop at position 32 relative to position 0, and a combination thereof. 396. The composition of embodiment 395, wherein the engineered guide RNA comprises at least about: 80%, 85%, 90%, 92%, 95%, 97%, or 99% sequence identity to SEQ ID NO: 930. 397. The composition of embodiment 396, wherein the engineered guide RNA comprises SEQ ID NO: 930. 398. The composition of embodiment 391, wherein the one or more structural features further comprises at least one structural feature selected from the group consisting of: an A/C mismatch at position 0, a second 6/6 symmetric internal loop at position 33 relative to position 0, and a combination thereof 399. The composition of embodiment 398, wherein the engineered guide RNA comprises at least about: 80%, 85%, 90%, 92%, 95%, 97%, or 99% sequence identity to SEQ ID NO: 934. 400. The composition of embodiment 399, wherein the engineered guide RNA comprises SEQ ID NO: 934. 401. The composition of embodiment 391, wherein the one or more structural features further comprises at least one structural feature selected from the group consisting of: an A/C mismatch at position 3 relative to position 0, a second 6/6 symmetric internal loop at position 35 relative to position 0, and a combination thereof. 402. The composition of embodiment 401, wherein the engineered guide RNA comprises at least about: 80%, 85%, 90%, 92%, 95%, 97%, or 99% sequence identity to SEQ ID NO: 944. 403. The composition of embodiment 402, wherein the engineered guide RNA comprises SEQ ID NO: 944. 404. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof. 405. The composition of embodiment 404, wherein the engineered guide RNA comprises at least about: 80%, 85%, 90%, 92%, 95%, 97%, or 99% sequence identity to SEQ ID NO: 1573. 406. The composition of embodiment 405, wherein the engineered guide RNA comprises SEQ ID NO: 1573. 407. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof. 408. The composition of embodiment 407, wherein the engineered guide RNA comprises at least about: 80%, 85%, 90%, 92%, 95%, 97%, or 99% sequence identity to SEQ ID NO: 1575. 409. The composition of embodiment 408, wherein the engineered guide RNA comprises SEQ ID NO: 1575. 410. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof. 411. The composition of embodiment 410, wherein the engineered guide RNA comprises at least about: 80%, 85%, 90%, 92%, 95%, 97%, or 99% sequence identity to SEQ ID NO: 1567. 412. The composition of embodiment 411, wherein the engineered guide RNA comprises SEQ ID NO: 1567. 413. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof. 414. The composition of embodiment 413, wherein the engineered guide RNA comprises at least about: 80%, 85%, 90%, 92%, 95%, 97%, or 99% sequence identity to SEQ ID NO: 1569. 415. The composition of embodiment 414, wherein the engineered guide RNA comprises SEQ ID NO: 1569. 416. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 47 relative to position 0, a 2/2 symmetric bulge at position 57 relative to position 0, a 2/2 symmetric bulge at position 67 relative to position 0, a 2/2 symmetric bulge at position 77 relative to position 0, and any combination thereof. 417. The composition of embodiment 416, wherein the engineered guide RNA comprises at least about: 80%, 85%, 90%, 92%, 95%, 97%, or 99% sequence identity to SEQ ID NO: 1570. 418. The composition of embodiment 417, wherein the engineered guide RNA comprises SEQ ID NO: 1570. 419. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 45 relative to position 0, a 2/2 symmetric bulge at position 53 relative to position 0, a 2/2 symmetric bulge at position 61 relative to position 0, a 2/2 symmetric bulge at position 69 relative to position 0, a 2/2 symmetric bulge at position 77 relative to position 0, and any combination thereof. 420. The composition of embodiment 419, wherein the engineered guide RNA comprises at least about: 80%, 85%, 90%, 92%, 95%, 97%, or 99% sequence identity to SEQ ID NO: 1566. 421. The composition of embodiment 420, wherein the engineered guide RNA comprises SEQ ID NO: 1566. 422. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 47 relative to position 0, a 4/4 symmetric bulge at position 59 relative to position 0, a 4/4 symmetric bulge at position 71 relative to position 0, and any combination thereof. 423. The composition of embodiment 422, wherein the engineered guide RNA comprises at least about: 80%, 85%, 90%, 92%, 95%, 97%, or 99% sequence identity to SEQ ID NO: 1572. 424. The composition of embodiment 423, wherein the engineered guide RNA comprises SEQ ID NO: 1572. 425. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 55 relative to position 0, a 3/3 symmetric bulge at position 74 relative to position 0, and any combination thereof. 426. The composition of embodiment 425, wherein the engineered guide RNA comprises at least about: 80%, 85%, 90%, 92%, 95%, 97%, or 99% sequence identity to SEQ ID NO: 1587. 427. The composition of embodiment 426, wherein the engineered guide RNA comprises SEQ ID NO: 1587. 428. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 47 relative to position 0, a 3/3 symmetric bulge at position 58 relative to position 0, a 3/3 symmetric bulge at position 69 relative to position 0, and any combination thereof. 429. The composition of embodiment 428, wherein the engineered guide RNA comprises at least about: 80%, 85%, 90%, 92%, 95%, 97%, or 99% sequence identity to SEQ ID NO: 1571. 430. The composition of embodiment 429, wherein the engineered guide RNA comprises SEQ ID NO: 1571. 431. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 49 relative to position 0, a 2/2 symmetric bulge at position 61 relative to position 0, a 2/2 symmetric bulge at position 73 relative to position 0, and any combination thereof. 432. The composition of embodiment 431, wherein the engineered guide RNA comprises at least about: 80%, 85%, 90%, 92%, 95%, 97%, or 99% sequence identity to SEQ ID NO: 1574. 433. The composition of embodiment 432, wherein the engineered guide RNA comprises SEQ ID NO: 1574. 434. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 53 relative to position 0, a 4/4 symmetric bulge at position 71 relative to position 0, and any combination thereof. 435. The composition of embodiment 434, wherein the engineered guide RNA comprises at least about: 80%, 85%, 90%, 92%, 95%, 97%, or 99% sequence identity to SEQ ID NO: 1584. 436. The composition of embodiment 435, wherein the engineered guide RNA comprises SEQ ID NO: 1584. 437. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof. 438. The composition of embodiment 437, wherein the engineered guide RNA comprises at least about: 80%, 85%, 90%, 92%, 95%, 97%, or 99% sequence identity to SEQ ID NO: 1588. 439. The composition of embodiment 438, wherein the engineered guide RNA comprises SEQ ID NO: 1588. 440. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 55 relative to position 0, a 2/2 symmetric bulge at position 73 relative to position 0, and any combination thereof. 441. The composition of embodiment 440, wherein the engineered guide RNA comprises at least about: 80%, 85%, 90%, 92%, 95%, 97%, or 99% sequence identity to SEQ ID NO: 1586. 442. The composition of embodiment 441, wherein the engineered guide RNA comprises SEQ ID NO: 1586. 443. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 53 relative to position 0, a 5/5 symmetric internal loop at position 72 relative to position 0, and any combination thereof. 444. The composition of embodiment 443, wherein the engineered guide RNA comprises at least about: 80%, 85%, 90%, 92%, 95%, 97%, or 99% sequence identity to SEQ ID NO: 1585. 445. The composition of embodiment 444, wherein the engineered guide RNA comprises SEQ ID NO: 1585. 446. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 51 relative to position 0, a 5/5 symmetric internal loop at position 68 relative to position 0, and any combination thereof. 447. The composition of embodiment 446, wherein the engineered guide RNA comprises at least about: 80%, 85%, 90%, 92%, 95%, 97%, or 99% sequence identity to SEQ ID NO: 1581. 448. The composition of embodiment 447, wherein the engineered guide RNA comprises SEQ ID NO: 1581. 449. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 51 relative to position 0, a 2/2 symmetric bulge at position 65 relative to position 0, and any combination thereof. 450. The composition of embodiment 449, wherein the engineered guide RNA comprises at least about: 80%, 85%, 90%, 92%, 95%, 97%, or 99% sequence identity to SEQ ID NO: 1578. 451. The composition of embodiment 450, wherein the engineered guide RNA comprises SEQ ID NO: 1578. 452. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 53 relative to position 0, a 2/2 symmetric bulge at position 69 relative to position 0, and any combination thereof. 453. The composition of embodiment 452, wherein the engineered guide RNA comprises at least about: 80%, 85%, 90%, 92%, 95%, 97%, or 99% sequence identity to SEQ ID NO: 1582. 454. The composition of embodiment 453, wherein the engineered guide RNA comprises SEQ ID NO: 1582. 455. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 51 relative to position 0, a 4/4 symmetric bulge at position 67 relative to position 0, and any combination thereof. 456. The composition of embodiment 455, wherein the engineered guide RNA comprises at least about: 80%, 85%, 90%, 92%, 95%, 97%, or 99% sequence identity to SEQ ID NO: 1580. 457. The composition of embodiment 456, wherein the engineered guide RNA comprises SEQ ID NO: 1580. 458. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 49 relative to position 0, a 5/5 symmetric internal loop at position 64 relative to position 0, and any combination thereof. 459. The composition of embodiment 458, wherein the engineered guide RNA comprises at least about: 80%, 85%, 90%, 92%, 95%, 97%, or 99% sequence identity to SEQ ID NO: 1577. 460. The composition of embodiment 459, wherein the engineered guide RNA comprises SEQ ID NO: 1577. 461. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 45 relative to position 0, a 4/4 symmetric bulge at position 55 relative to position 0, a 4/4 symmetric bulge at position 65 relative to position 0, a 4/4 symmetric bulge at position 75 relative to position 0, and any combination thereof. 462. The composition of embodiment 461, wherein the engineered guide RNA comprises at least about: 80%, 85%, 90%, 92%, 95%, 97%, or 99% sequence identity to SEQ ID NO: 1568. 463. The composition of embodiment 462, wherein the engineered guide RNA comprises SEQ ID NO: 1568. 464. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 51 relative to position 0, a 3/3 symmetric bulge at position 66 relative to position 0, and any combination thereof. 465. The composition of embodiment 464, wherein the engineered guide RNA comprises at least about: 80%, 85%, 90%, 92%, 95%, 97%, or 99% sequence identity to SEQ ID NO: 1579. 466. The composition of embodiment 465, wherein the engineered guide RNA comprises SEQ ID NO: 1579. 467. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 53 relative to position 0, a 3/3 symmetric bulge at position 70 relative to position 0, and any combination thereof. 468. The composition of embodiment 467, wherein the engineered guide RNA comprises at least about: 80%, 85%, 90%, 92%, 95%, 97%, or 99% sequence identity to SEQ ID NO: 1583. 469. The composition of embodiment 468, wherein the engineered guide RNA comprises SEQ ID NO: 1583. 470. The composition of embodiment 391, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 49 relative to position 0, a 4/4 symmetric bulge at position 63 relative to position 0, and any combination thereof. 471. The composition of embodiment 470, wherein the engineered guide RNA comprises at least about: 80%, 85%, 90%, 92%, 95%, 97%, or 99% sequence identity to SEQ ID NO: 1576. 472. The composition of embodiment 471, wherein the engineered guide RNA comprises SEQ ID NO: 1576. 473. The composition of embodiment 339, wherein the first 6/6 symmetric internal loop is at position −7 relative to position 0. 474. The composition of embodiment 473, wherein the one or more structural features further comprises at least one structural feature selected from the group consisting of: an A/C mismatch at position 3 relative to position 0, a second 6/6 symmetric internal loop at position 34 relative to position 0, and a combination thereof. 475. The composition of embodiment 474, wherein the engineered guide RNA comprises at least about: 80%, 85%, 90%, 92%, 95%, 97%, or 99% sequence identity to SEQ ID NO: 815. 476. The composition of embodiment 475, wherein the engineered guide RNA comprises SEQ ID NO: 815. 477. The composition of embodiment 473, wherein the one or more structural features further comprises at least one structural feature selected from the group consisting of: an A/C mismatch at position 4 relative to position 0, a second 6/6 symmetric internal loop at position 32 relative to position 0, and a combination thereof 478. The composition of embodiment 477, wherein the engineered guide RNA comprises at least about: 80%, 85%, 90%, 92%, 95%, 97%, or 99% sequence identity to SEQ ID NO: 806. 479. The composition of embodiment 478, wherein the engineered guide RNA comprises SEQ ID NO: 806. 480. The composition of embodiment 339, wherein the first 6/6 symmetric internal loop is at position −8 relative to position 0. 481. The composition of embodiment 480, wherein the one or more structural features further comprises at least one structural feature selected from the group consisting of: an A/C mismatch at position 3 relative to position 0, a second 6/6 symmetric internal loop at position 35 relative to position 0, and a combination thereof. 482. The composition of embodiment 481, wherein the engineered guide RNA comprises at least about: 80%, 85%, 90%, 92%, 95%, 97%, or 99% sequence identity to SEQ ID NO: 694. 483. The composition of embodiment 482, wherein the engineered guide RNA comprises SEQ ID NO: 694. 484. The composition of embodiment 339, wherein the first 6/6 symmetric internal loop is at position −9 relative to position 0. 485. The composition of embodiment 484, wherein the one or more structural features further comprises at least one structural feature selected from the group consisting of: an A/C mismatch at position 0, a second 6/6 symmetric internal loop at position 40 relative to position 0, and a combination thereof. 486. The composition of embodiment 485, wherein the engineered guide RNA comprises at least about: 80%, 85%, 90%, 92%, 95%, 97%, or 99% sequence identity to SEQ ID NO: 593. 487. The composition of embodiment 486, wherein the engineered guide RNA comprises SEQ ID NO: 593. 488. The composition of embodiment 484, wherein the one or more structural features further comprises at least one structural feature selected from the group consisting of: an A/C mismatch at position 3 relative to position 0, a second 6/6 symmetric internal loop at position 34 relative to position 0, and a combination thereof 489. The composition of embodiment 488, wherein the engineered guide RNA comprises at least about: 80%, 85%, 90%, 92%, 95%, 97%, or 99% sequence identity to SEQ ID NO: 566. 490. The composition of embodiment 489, wherein the engineered guide RNA comprises SEQ ID NO: 566. 491. The composition of embodiment 484, wherein the one or more structural features further comprises at least one structural feature selected from the group consisting of: an A/C mismatch at position 3 relative to position 0, a second 6/6 symmetric internal loop at position 40 relative to position 0, and a combination thereof 492. The composition of embodiment 491, wherein the engineered guide RNA comprises at least about: 80%, 85%, 90%, 92%, 95%, 97%, or 99% sequence identity to SEQ ID NO: 594. 493. The composition of embodiment 492, wherein the engineered guide RNA comprises SEQ ID NO: 594. 494. The composition of embodiment 339, wherein the first 6/6 symmetric internal loop is at position −11 relative to position 0. 495. The composition of embodiment 494, wherein the one or more structural features further comprises at least one structural feature selected from the group consisting of: an A/C mismatch at position 4 relative to position 0, a second 6/6 symmetric internal loop at position 42 relative to position 0, and a combination thereof. 496. The composition of embodiment 495, wherein the engineered guide RNA comprises at least about: 80%, 85%, 90%, 92%, 95%, 97%, or 99% sequence identity to SEQ ID NO: 358. 497. The composition of embodiment 496, wherein the engineered guide RNA comprises SEQ ID NO: 358. 498. The composition of embodiment 339, wherein the first 6/6 symmetric internal loop is at position −12 relative to position 0. 499. The composition of embodiment 498, wherein the one or more structural features further comprises at least one structural feature selected from the group consisting of: an A/C mismatch at position 5 relative to position 0, a second 6/6 symmetric internal loop at position 33 relative to position 0, and a combination thereof 500. The composition of embodiment 499, wherein the engineered guide RNA comprises at least about: 80%, 85%, 90%, 92%, 95%, 97%, or 99% sequence identity to SEQ ID NO: 195. 501. The composition of embodiment 500, wherein the engineered guide RNA comprises SEQ ID NO: 195. 502. The composition of any one of embodiments 6-9, wherein position 5 of ATTAAA is the target adenosine, wherein position 5 is the forth A of ATTAAA from the 5′ end. 503. The composition of embodiment 340, wherein the one or more structural features comprises: a first 6/6 symmetric internal loop at a position selected from the group consisting of: 33, 23, −1, −2, −3, −4, −5, −6, −7, −8, −9, −10, and −12 relative to position 0 of ATTAAA. 504. The composition of embodiment 503, wherein the first 6/6 symmetric internal loop is at position 33 relative to position 0. 505. The composition of embodiment 504, wherein the one or more structural features further comprises an A/C mismatch at position 3 relative to position 0. 506. The composition of embodiment 505, wherein the engineered guide RNA comprises at least about: 80%, 85%, 90%, 92%, 95%, 97%, or 99% sequence identity to SEQ ID NO: 72. 507. The composition of embodiment 506, wherein the engineered guide RNA comprises SEQ ID NO: 72. 508. The composition of embodiment 503, wherein the first 6/6 symmetric internal loop is at position 23 relative to position 0. 509. The composition of embodiment 508, wherein the one or more structural features further comprises an A/C mismatch at position 5 relative to position 0. 510. The composition of embodiment 509, wherein the engineered guide RNA comprises at least about: 80%, 85%, 90%, 92%, 95%, 97%, or 99% sequence identity to SEQ ID NO: 24. 511. The composition of embodiment 510, wherein the engineered guide RNA comprises SEQ ID NO: 24. 512. The composition of embodiment 503, wherein the first 6/6 symmetric internal loop is at position −1 relative to position 0. 513. The composition of embodiment 512, wherein the one or more structural features further comprises at least one structural feature selected from the group consisting of: an A/C mismatch at position 4 relative to position 0, a second 6/6 symmetric internal loop at position 32 relative to position 0, and a combination thereof. 514. The composition of embodiment 513, wherein the engineered guide RNA comprises at least about: 80%, 85%, 90%, 92%, 95%, 97%, or 99% sequence identity to SEQ ID NO: 1463. 515. The composition of embodiment 514, wherein the engineered guide RNA comprises SEQ ID NO: 1463. 516. The composition of embodiment 503, wherein the first 6/6 symmetric internal loop is at position −2 relative to position 0. 517. The composition of embodiment 516, wherein the one or more structural features further comprises at least one structural feature selected from the group consisting of: an A/C mismatch at position 5 relative to position 0, a second 6/6 symmetric internal loop at position 42 relative to position 0, and a combination thereof. 518. The composition of embodiment 517, wherein the engineered guide RNA comprises at least about: 80%, 85%, 90%, 92%, 95%, 97%, or 99% sequence identity to SEQ ID NO: 1411. 519. The composition of embodiment 518, wherein the engineered guide RNA comprises SEQ ID NO: 1411. 520. The composition of embodiment 516, wherein the one or more structural features further comprises at least one structural feature selected from the group consisting of: an A/C mismatch at position 3 relative to position 0, a second 6/6 symmetric internal loop at position 37 relative to position 0, and a combination thereof. 521. The composition of embodiment 520, wherein the engineered guide RNA comprises at least about: 80%, 85%, 90%, 92%, 95%, 97%, or 99% sequence identity to SEQ ID NO: 1391. 522. The composition of embodiment 521, wherein the engineered guide RNA comprises SEQ ID NO: 1391. 523. The composition of embodiment 503, wherein the first 6/6 symmetric internal loop is at position −3 relative to position 0. 524. The composition of embodiment 523, wherein the one or more structural features further comprises at least one structural feature selected from the group consisting of: an A/C mismatch at position 3 relative to position 0, a second 6/6 symmetric internal loop at position 36 relative to position 0, and a combination thereof 525. The composition of embodiment 524, wherein the engineered guide RNA comprises at least about: 80%, 85%, 90%, 92%, 95%, 97%, or 99% sequence identity to SEQ ID NO: 1293. 526. The composition of embodiment 525, wherein the engineered guide RNA comprises SEQ ID NO: 1293. 527. The composition of embodiment 503, wherein the first 6/6 symmetric internal loop is at position −4 relative to position 0. 528. The composition of embodiment 527, wherein the one or more structural features further comprises at least one structural feature selected from the group consisting of: an A/C mismatch at position 5 relative to position 0, a second 6/6 symmetric internal loop at position 36 relative to position 0, and a combination thereof. 529. The composition of embodiment 528, wherein the engineered guide RNA comprises at least about: 80%, 85%, 90%, 92%, 95%, 97%, or 99% sequence identity to SEQ ID NO: 1185. 530. The composition of embodiment 529, wherein the engineered guide RNA comprises SEQ ID NO: 1185. 531. The composition of embodiment 527, wherein the one or more structural features further comprises at least one structural feature selected from the group consisting of: an A/C mismatch at position 3 relative to position 0, a second 6/6 symmetric internal loop at position 32 relative to position 0, and a combination thereof. 532. The composition of embodiment 531, wherein the engineered guide RNA comprises at least about: 80%, 85%, 90%, 92%, 95%, 97%, or 99% sequence identity to SEQ ID NO: 1163. 533. The composition of embodiment 532, wherein the engineered guide RNA comprises SEQ ID NO: 1163. 534. The composition of embodiment 527, wherein the one or more structural features further comprises at least one structural feature selected from the group consisting of: an A/C mismatch at position 3 relative to position 0, a second 6/6 symmetric internal loop at position 36 relative to position 0, and a combination thereof. 535. The composition of embodiment 534, wherein the engineered guide RNA comprises at least about: 80%, 85%, 90%, 92%, 95%, 97%, or 99% sequence identity to SEQ ID NO: 1183. 536. The composition of embodiment 535, wherein the engineered guide RNA comprises SEQ ID NO: 1183. 537. The composition of embodiment 527, wherein the one or more structural features further comprises at least one structural feature selected from the group consisting of: an A/C mismatch at position 3 relative to position 0, a second 6/6 symmetric internal loop at position 42 relative to position 0, and a combination thereof. 538. The composition of embodiment 537, wherein the engineered guide RNA comprises at least about: 80%, 85%, 90%, 92%, 95%, 97%, or 99% sequence identity to SEQ ID NO: 1212. 539. The composition of embodiment 538, wherein the engineered guide RNA comprises SEQ ID NO: 1212. 540. The composition of embodiment 527, wherein the one or more structural features further comprises at least one structural feature selected from the group consisting of: an A/C mismatch at position 3 relative to position 0, a second 6/6 symmetric internal loop at position 33 relative to position 0, and a combination thereof. 541. The composition of embodiment 540, wherein the engineered guide RNA comprises at least about: 80%, 85%, 90%, 92%, 95%, 97%, or 99% sequence identity to SEQ ID NO: 1168. 542. The composition of embodiment 541, wherein the engineered guide RNA comprises SEQ ID NO: 1168. 543. The composition of embodiment 503, wherein the first 6/6 symmetric internal loop is at position −5 relative to position 0. 544. The composition of embodiment 543, wherein the one or more structural features further comprises at least one structural feature selected from the group consisting of: an A/C mismatch at position 3 relative to position 0, a second 6/6 symmetric internal loop at position 36 relative to position 0, and a combination thereof. 545. The composition of embodiment 544, wherein the engineered guide RNA comprises at least about: 80%, 85%, 90%, 92%, 95%, 97%, or 99% sequence identity to SEQ ID NO: 1066. 546. The composition of embodiment 545, wherein the engineered guide RNA comprises SEQ ID NO: 1066. 547. The composition of embodiment 543, wherein the one or more structural features further comprises at least one structural feature selected from the group consisting of: an A/C mismatch at position 5 relative to position 0, a second 6/6 symmetric internal loop at position 34 relative to position 0, and a combination thereof. 548. The composition of embodiment 547, wherein the engineered guide RNA comprises at least about: 80%, 85%, 90%, 92%, 95%, 97%, or 99% sequence identity to SEQ ID NO: 1058. 549. The composition of embodiment 548, wherein the engineered guide RNA comprises SEQ ID NO: 1058. 550. The composition of embodiment 543, wherein the one or more structural features further comprises at least one structural feature selected from the group consisting of: an A/C mismatch at position 3 relative to position 0, a second 6/6 symmetric internal loop at position 33 relative to position 0, and a combination thereof. 551. The composition of embodiment 550, wherein the engineered guide RNA comprises at least about: 80%, 85%, 90%, 92%, 95%, 97%, or 99% sequence identity to SEQ ID NO: 1051. 552. The composition of embodiment 551, wherein the engineered guide RNA comprises SEQ ID NO: 1051. 553. The composition of embodiment 543, wherein the one or more structural features further comprises a second 6/6 symmetric internal loop at position 33 relative to position 0. 554. The composition of embodiment 553, wherein the engineered guide RNA comprises at least about: 80%, 85%, 90%, 92%, 95%, 97%, or 99% sequence identity to SEQ ID NO: 1054. 555. The composition of embodiment 554, wherein the engineered guide RNA comprises SEQ ID NO: 1054. 556. The composition of embodiment 543, wherein the one or more structural features further comprises at least one structural feature selected from the group consisting of: an A/C mismatch at position 3 relative to position 0, a second 6/6 symmetric internal loop at position 44 relative to position 0, and a combination thereof 557. The composition of embodiment 556, wherein the engineered guide RNA comprises at least about: 80%, 85%, 90%, 92%, 95%, 97%, or 99% sequence identity to SEQ ID NO: 1104. 558. The composition of embodiment 557, wherein the engineered guide RNA comprises SEQ ID NO: 1104. 559. The composition of embodiment 503, wherein the first 6/6 symmetric internal loop is at position −6 relative to position 0. 560. The composition of embodiment 559, wherein the one or more structural features further comprises at least one structural feature selected from the group consisting of: an A/C mismatch at position 3 relative to position 0, a second 6/6 symmetric internal loop at position 32 relative to position 0, and a combination thereof. 561. The composition of embodiment 560, wherein the engineered guide RNA comprises at least about: 80%, 85%, 90%, 92%, 95%, 97%, or 99% sequence identity to SEQ ID NO: 930. 562. The composition of embodiment 561, wherein the engineered guide RNA comprises SEQ ID NO: 930. 563. The composition of embodiment 559, wherein the one or more structural features further comprises at least one structural feature selected from the group consisting of: an A/C mismatch at position 3 relative to position 0, a second 6/6 symmetric internal loop at position 35 relative to position 0, and a combination thereof 564. The composition of embodiment 563, wherein the engineered guide RNA comprises at least about: 80%, 85%, 90%, 92%, 95%, 97%, or 99% sequence identity to SEQ ID NO: 944. 565. The composition of embodiment 564, wherein the engineered guide RNA comprises SEQ ID NO: 944. 566. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, and a combination thereof 567. The composition of embodiment 566, wherein the engineered guide RNA comprises at least about: 80%, 85%, 90%, 92%, 95%, 97%, or 99% sequence identity to SEQ ID NO: 935. 568. The composition of embodiment 567, wherein the engineered guide RNA comprises SEQ ID NO: 935. 569. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof. 570. The composition of embodiment 569, wherein the engineered guide RNA comprises at least about: 80%, 85%, 90%, 92%, 95%, 97%, or 99% sequence identity to SEQ ID NO: 1575. 571. The composition of embodiment 570, wherein the engineered guide RNA comprises SEQ ID NO: 1575. 572. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof. 573. The composition of embodiment 572, wherein the engineered guide RNA comprises at least about: 80%, 85%, 90%, 92%, 95%, 97%, or 99% sequence identity to SEQ ID NO: 1567. 574. The composition of embodiment 573, wherein the engineered guide RNA comprises SEQ ID NO: 1567. 575. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 47 relative to position 0, a 3/3 symmetric bulge at position 58 relative to position 0, a 3/3 symmetric bulge at position 69 relative to position 0, and any combination thereof. 576. The composition of embodiment 575, wherein the engineered guide RNA comprises at least about: 80%, 85%, 90%, 92%, 95%, 97%, or 99% sequence identity to SEQ ID NO: 1571. 577. The composition of embodiment 576, wherein the engineered guide RNA comprises SEQ ID NO: 1571. 578. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 55 relative to position 0, a 3/3 symmetric bulge at position 74 relative to position 0, and any combination thereof. 579. The composition of embodiment 578, wherein the engineered guide RNA comprises at least about: 80%, 85%, 90%, 92%, 95%, 97%, or 99% sequence identity to SEQ ID NO: 1587. 580. The composition of embodiment 579, wherein the engineered guide RNA comprises SEQ ID NO: 1587. 581. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 47 relative to position 0, a 2/2 symmetric bulge at position 57 relative to position 0, a 2/2 symmetric bulge at position 67 relative to position 0, a 2/2 symmetric bulge at position 77 relative to position 0, and any combination thereof. 582. The composition of embodiment 581, wherein the engineered guide RNA comprises at least about: 80%, 85%, 90%, 92%, 95%, 97%, or 99% sequence identity to SEQ ID NO: 1570. 583. The composition of embodiment 582, wherein the engineered guide RNA comprises SEQ ID NO: 1570. 584. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 45 relative to position 0, a 2/2 symmetric bulge at position 53 relative to position 0, a 2/2 symmetric bulge at position 61 relative to position 0, a 2/2 symmetric bulge at position 69 relative to position 0, a 2/2 symmetric bulge at position 77 relative to position 0, and any combination thereof. 585. The composition of embodiment 584, wherein the engineered guide RNA comprises at least about: 80%, 85%, 90%, 92%, 95%, 97%, or 99% sequence identity to SEQ ID NO: 1566. 586. The composition of embodiment 585, wherein the engineered guide RNA comprises SEQ ID NO: 1566. 587. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 49 relative to position 0, a 2/2 symmetric bulge at position 61 relative to position 0, a 2/2 symmetric bulge at position 73 relative to position 0, and any combination thereof. 588. The composition of embodiment 587, wherein the engineered guide RNA comprises at least about: 80%, 85%, 90%, 92%, 95%, 97%, or 99% sequence identity to SEQ ID NO: 1574. 589. The composition of embodiment 588, wherein the engineered guide RNA comprises SEQ ID NO: 1574. 590. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 51 relative to position 0, a 2/2 symmetric bulge at position 65 relative to position 0, and any combination thereof. 591. The composition of embodiment 590, wherein the engineered guide RNA comprises at least about: 80%, 85%, 90%, 92%, 95%, 97%, or 99% sequence identity to SEQ ID NO: 1578. 592. The composition of embodiment 591, wherein the engineered guide RNA comprises SEQ ID NO: 1578. 593. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 55 relative to position 0, a 2/2 symmetric bulge at position 73 relative to position 0, and any combination thereof. 594. The composition of embodiment 593, wherein the engineered guide RNA comprises at least about: 80%, 85%, 90%, 92%, 95%, 97%, or 99% sequence identity to SEQ ID NO: 1586. 595. The composition of embodiment 594, wherein the engineered guide RNA comprises SEQ ID NO: 1586. 596. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 2/2 symmetric bulge at position 53 relative to position 0, a 2/2 symmetric bulge at position 69 relative to position 0, and any combination thereof. 597. The composition of embodiment 596, wherein the engineered guide RNA comprises at least about: 80%, 85%, 90%, 92%, 95%, 97%, or 99% sequence identity to SEQ ID NO: 1582. 598. The composition of embodiment 597, wherein the engineered guide RNA comprises SEQ ID NO: 1582. 599. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof. 600. The composition of embodiment 599, wherein the engineered guide RNA comprises at least about: 80%, 85%, 90%, 92%, 95%, 97%, or 99% sequence identity to SEQ ID NO: 1573. 601. The composition of embodiment 600, wherein the engineered guide RNA comprises SEQ ID NO: 1573. 602. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 51 relative to position 0, a 5/5 symmetric internal loop at position 68 relative to position 0, and any combination thereof. 603. The composition of embodiment 602, wherein the engineered guide RNA comprises at least about: 80%, 85%, 90%, 92%, 95%, 97%, or 99% sequence identity to SEQ ID NO: 1581. 604. The composition of embodiment 603, wherein the engineered guide RNA comprises SEQ ID NO: 1581. 605. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof. 606. The composition of embodiment 605, wherein the engineered guide RNA comprises at least about: 80%, 85%, 90%, 92%, 95%, 97%, or 99% sequence identity to SEQ ID NO: 1569. 607. The composition of embodiment 606, wherein the engineered guide RNA comprises SEQ ID NO: 1569. 608. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 53 relative to position 0, a 5/5 symmetric internal loop at position 72 relative to position 0, and any combination thereof. 609. The composition of embodiment 608, wherein the engineered guide RNA comprises at least about: 80%, 85%, 90%, 92%, 95%, 97%, or 99% sequence identity to SEQ ID NO: 1585. 610. The composition of embodiment 609, wherein the engineered guide RNA comprises SEQ ID NO: 1585. 611. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 51 relative to position 0, a 4/4 symmetric bulge at position 67 relative to position 0, and any combination thereof. 612. The composition of embodiment 611, wherein the engineered guide RNA comprises at least about: 80%, 85%, 90%, 92%, 95%, 97%, or 99% sequence identity to SEQ ID NO: 1580. 613. The composition of embodiment 612, wherein the engineered guide RNA comprises SEQ ID NO: 1580. 614. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof. 615. The composition of embodiment 614, wherein the engineered guide RNA comprises at least about: 80%, 85%, 90%, 92%, 95%, 97%, or 99% sequence identity to SEQ ID NO: 1588. 616. The composition of embodiment 615, wherein the engineered guide RNA comprises SEQ ID NO: 1588. 617. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 53 relative to position 0, a 4/4 symmetric bulge at position 71 relative to position 0, and any combination thereof. 618. The composition of embodiment 617, wherein the engineered guide RNA comprises at least about: 80%, 85%, 90%, 92%, 95%, 97%, or 99% sequence identity to SEQ ID NO: 1584. 619. The composition of embodiment 618, wherein the engineered guide RNA comprises SEQ ID NO: 1584. 620. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 49 relative to position 0, a 4/4 symmetric bulge at position 63 relative to position 0, and any combination thereof. 621. The composition of embodiment 620, wherein the engineered guide RNA comprises at least about: 80%, 85%, 90%, 92%, 95%, 97%, or 99% sequence identity to SEQ ID NO: 1576. 622. The composition of embodiment 621, wherein the engineered guide RNA comprises SEQ ID NO: 1576. 623. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 47 relative to position 0, a 4/4 symmetric bulge at position 59 relative to position 0, a 4/4 symmetric bulge at position 71 relative to position 0, and any combination thereof. 624. The composition of embodiment 623, wherein the engineered guide RNA comprises at least about: 80%, 85%, 90%, 92%, 95%, 97%, or 99% sequence identity to SEQ ID NO: 1572. 625. The composition of embodiment 624, wherein the engineered guide RNA comprises SEQ ID NO: 1572. 626. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 45 relative to position 0, a 4/4 symmetric bulge at position 55 relative to position 0, a 4/4 symmetric bulge at position 65 relative to position 0, a 4/4 symmetric bulge at position 75 relative to position 0, and any combination thereof. 627. The composition of embodiment 626, wherein the engineered guide RNA comprises at least about: 80%, 85%, 90%, 92%, 95%, 97%, or 99% sequence identity to SEQ ID NO: 1568. 628. The composition of embodiment 627, wherein the engineered guide RNA comprises SEQ ID NO: 1568. 629. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 49 relative to position 0, a 5/5 symmetric internal loop at position 64 relative to position 0, and any combination thereof. 630. The composition of embodiment 629, wherein the engineered guide RNA comprises at least about: 80%, 85%, 90%, 92%, 95%, 97%, or 99% sequence identity to SEQ ID NO: 1577. 631. The composition of embodiment 630, wherein the engineered guide RNA comprises SEQ ID NO: 1577. 632. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 51 relative to position 0, a 3/3 symmetric bulge at position 66 relative to position 0, and any combination thereof. 633. The composition of embodiment 632, wherein the engineered guide RNA comprises at least about: 80%, 85%, 90%, 92%, 95%, 97%, or 99% sequence identity to SEQ ID NO: 1579. 634. The composition of embodiment 633, wherein the engineered guide RNA comprises SEQ ID NO: 1579. 635. The composition of embodiment 559, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 53 relative to position 0, a 3/3 symmetric bulge at position 70 relative to position 0, and any combination thereof. 636. The composition of embodiment 635, wherein the engineered guide RNA comprises at least about: 80%, 85%, 90%, 92%, 95%, 97%, or 99% sequence identity to SEQ ID NO: 1583. 637. The composition of embodiment 636, wherein the engineered guide RNA comprises SEQ ID NO: 1583. 638. The composition of embodiment 503, wherein the first 6/6 symmetric internal loop is at position −7 relative to position 0. 639. The composition of embodiment 638, wherein the one or more structural features further comprises at least one structural feature selected from the group consisting of: an A/C mismatch at position 3 relative to position 0, a second 6/6 symmetric internal loop at position 33 relative to position 0, and a combination thereof. 640. The composition of embodiment 639, wherein the engineered guide RNA comprises at least about: 80%, 85%, 90%, 92%, 95%, 97%, or 99% sequence identity to SEQ ID NO: 810. 641. The composition of embodiment 640, wherein the engineered guide RNA comprises SEQ ID NO: 810. 642. The composition of embodiment 638, wherein the one or more structural features further comprises at least one structural feature selected from the group consisting of: an A/C mismatch at position 3 relative to position 0, a second 6/6 symmetric internal loop at position 34 relative to position 0, and a combination thereof. 643. The composition of embodiment 642, wherein the engineered guide RNA comprises at least about: 80%, 85%, 90%, 92%, 95%, 97%, or 99% sequence identity to SEQ ID NO: 815. 644. The composition of embodiment 643, wherein the engineered guide RNA comprises SEQ ID NO: 815. 645. The composition of embodiment 503, wherein the first 6/6 symmetric internal loop is at position −8 relative to position 0. 646. The composition of embodiment 645, wherein the one or more structural features further comprises at least one structural feature selected from the group consisting of: an A/C mismatch at position 3 relative to position 0, a second 6/6 symmetric internal loop at position 32 relative to position 0, and a combination thereof. 647. The composition of embodiment 646, wherein the engineered guide RNA comprises at least about: 80%, 85%, 90%, 92%, 95%, 97%, or 99% sequence identity to SEQ ID NO: 680. 648. The composition of embodiment 647, wherein the engineered guide RNA comprises SEQ ID NO: 680. 649. The composition of embodiment 645, wherein the one or more structural features further comprises at least one structural feature selected from the group consisting of: an A/C mismatch at position 3 relative to position 0, a second 6/6 symmetric internal loop at position 35 relative to position 0, and a combination thereof. 650. The composition of embodiment 649, wherein the engineered guide RNA comprises at least about: 80%, 85%, 90%, 92%, 95%, 97%, or 99% sequence identity to SEQ ID NO: 694. 651. The composition of embodiment 650, wherein the engineered guide RNA comprises SEQ ID NO: 694. 652. The composition of embodiment 503, wherein the first 6/6 symmetric internal loop is at position −9 relative to position 0. 653. The composition of embodiment 652, wherein the one or more structural features further comprises at least one structural feature selected from the group consisting of: an A/C mismatch at position 3 relative to position 0, a second 6/6 symmetric internal loop at position 34 relative to position 0, and a combination thereof. 654. The composition of embodiment 653, wherein the engineered guide RNA comprises at least about: 80%, 85%, 90%, 92%, 95%, 97%, or 99% sequence identity to SEQ ID NO: 566. 655. The composition of embodiment 654, wherein the engineered guide RNA comprises SEQ ID NO: 566. 656. The composition of embodiment 652, wherein the one or more structural features further comprises at least one structural feature selected from the group consisting of: an A/C mismatch at position 3 relative to position 0, a second 6/6 symmetric internal loop at position 40 relative to position 0, and a combination thereof. 657. The composition of embodiment 656, wherein the engineered guide RNA comprises at least about: 80%, 85%, 90%, 92%, 95%, 97%, or 99% sequence identity to SEQ ID NO: 594. 658. The composition of embodiment 657, wherein the engineered guide RNA comprises SEQ ID NO: 594. 659. The composition of embodiment 503, wherein the first 6/6 symmetric internal loop is at position −10 relative to position 0. 660. The composition of embodiment 659, wherein the one or more structural features further comprises at least one structural feature selected from the group consisting of: an A/C mismatch at position 5 relative to position 0, a second 6/6 symmetric internal loop at position 23 relative to position 0, and a combination thereof. 661. The composition of embodiment 660, wherein the engineered guide RNA comprises at least about: 80%, 85%, 90%, 92%, 95%, 97%, or 99% sequence identity to SEQ ID NO: 392. 662. The composition of embodiment 661, wherein the engineered guide RNA comprises SEQ ID NO: 392. 663. The composition of embodiment 503, wherein the first 6/6 symmetric internal loop is at position −12 relative to position 0. 664. The composition of embodiment 663, wherein the one or more structural features further comprises at least one structural feature selected from the group consisting of: an A/C mismatch at position 5 relative to position 0, a second 6/6 symmetric internal loop at position 33 relative to position 0, and a combination thereof 665. The composition of embodiment 664, wherein the engineered guide RNA comprises at least about: 80%, 85%, 90%, 92%, 95%, 97%, or 99% sequence identity to SEQ ID NO: 195. 666. The composition of embodiment 665, wherein the engineered guide RNA comprises SEQ ID NO: 195. 667. The composition of any one of embodiments 10-666, further comprising editing at any A of ATTAAA. 668. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 669. The composition of embodiment 668, wherein the engineered guide RNA has at least 80%, 85%, 90%, 92%, 95%, 97%, or 99% sequence identity to a guide RNA comprising SEQ ID NO: 8. 670. The composition of any one of embodiments 668-669, wherein the engineered guide RNA has a sequence of SEQ ID NO: 8. 671. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 672. The composition of embodiment 671, wherein the engineered guide RNA has at least 80%, 85%, 90%, 92%, 95%, 97%, or 99% sequence identity to a guide RNA comprising SEQ ID NO: 10. 673. The composition of any one of embodiments 671-672, wherein the engineered guide RNA has a sequence of SEQ ID NO: 10. 674. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 675. The composition of embodiment 674, wherein the engineered guide RNA has at least 80%, 85%, 90%, 92%, 95%, 97%, or 99% sequence identity to a guide RNA comprising SEQ ID NO: 14. 676. The composition of any one of embodiments 674-675, wherein the engineered guide RNA has a sequence of SEQ ID NO: 14. 677. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 678. The composition of embodiment 677, wherein the engineered guide RNA has at least 80%, 85%, 90%, 92%, 95%, 97%, or 99% sequence identity to a guide RNA comprising SEQ ID NO: 15. 679. The composition of any one of embodiments 677-678, wherein the engineered guide RNA has a sequence of SEQ ID NO: 15. 680. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 681. The composition of embodiment 680, wherein the engineered guide RNA has at least 80%, 85%, 90%, 92%, 95%, 97%, or 99% sequence identity to a guide RNA comprising SEQ ID NO: 17. 682. The composition of any one of embodiments 680-681, wherein the engineered guide RNA has a sequence of SEQ ID NO: 17. 683. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 684. The composition of embodiment 683, wherein the engineered guide RNA has at least 80%, 85%, 90%, 92%, 95%, 97%, or 99% sequence identity to a guide RNA comprising SEQ ID NO: 24. 685. The composition of any one of embodiments 683-684, wherein the engineered guide RNA has a sequence of SEQ ID NO: 24. 686. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 687. The composition of embodiment 686, wherein the engineered guide RNA has at least 80%, 85%, 90%, 92%, 95%, 97%, or 99% sequence identity to a guide RNA comprising SEQ ID NO: 72. 688. The composition of any one of embodiments 686-687, wherein the engineered guide RNA has a sequence of SEQ ID NO: 72. 689. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 690. The composition of embodiment 689, wherein the engineered guide RNA has at least 80%, 85%, 90%, 92%, 95%, 97%, or 99% sequence identity to a guide RNA comprising SEQ ID NO: 195. 691. The composition of any one of embodiments 689-690, wherein the engineered guide RNA has a sequence of SEQ ID NO: 195. 692. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 693. The composition of embodiment 692, wherein the engineered guide RNA has at least 80%, 85%, 90%, 92%, 95%, 97%, or 99% sequence identity to a guide RNA comprising SEQ ID NO: 252. 694. The composition of any one of embodiments 692-693, wherein the engineered guide RNA has a sequence of SEQ ID NO: 252. 695. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 696. The composition of embodiment 695, wherein the engineered guide RNA has at least 80%, 85%, 90%, 92%, 95%, 97%, or 99% sequence identity to a guide RNA comprising SEQ ID NO: 291. 697. The composition of any one of embodiments 695-696, wherein the engineered guide RNA has a sequence of SEQ ID NO: 291. 698. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 699. The composition of embodiment 698, wherein the engineered guide RNA has at least 80%, 85%, 90%, 92%, 95%, 97%, or 99% sequence identity to a guide RNA comprising SEQ ID NO: 352. 700. The composition of any one of embodiments 698-699, wherein the engineered guide RNA has a sequence of SEQ ID NO: 352. 701. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 702. The composition of embodiment 701, wherein the engineered guide RNA has at least 80%, 85%, 90%, 92%, 95%, 97%, or 99% sequence identity to a guide RNA comprising SEQ ID NO: 356. 703. The composition of any one of embodiments 701-702, wherein the engineered guide RNA has a sequence of SEQ ID NO: 356. 704. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 705. The composition of embodiment 704, wherein the engineered guide RNA has at least 80%, 85%, 90%, 92%, 95%, 97%, or 99% sequence identity to a guide RNA comprising SEQ ID NO: 358. 706. The composition of any one of embodiments 704-705, wherein the engineered guide RNA has a sequence of SEQ ID NO: 358. 707. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 708. The composition of embodiment 707, wherein the engineered guide RNA has at least 80%, 85%, 90%, 92%, 95%, 97%, or 99% sequence identity to a guide RNA comprising SEQ ID NO: 365. 709. The composition of any one of embodiments 707-708, wherein the engineered guide RNA has a sequence of SEQ ID NO: 365. 710. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 711. The composition of embodiment 710, wherein the engineered guide RNA has at least 80%, 85%, 90%, 92%, 95%, 97%, or 99% sequence identity to a guide RNA comprising SEQ ID NO: 375. 712. The composition of any one of embodiments 710-711, wherein the engineered guide RNA has a sequence of SEQ ID NO: 375. 713. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 714. The composition of embodiment 713, wherein the engineered guide RNA has at least 80%, 85%, 90%, 92%, 95%, 97%, or 99% sequence identity to a guide RNA comprising SEQ ID NO: 392. 715. The composition of any one of embodiments 713-714, wherein the engineered guide RNA has a sequence of SEQ ID NO: 392. 716. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 717. The composition of embodiment 716, wherein the engineered guide RNA has at least 80%, 85%, 90%, 92%, 95%, 97%, or 99% sequence identity to a guide RNA comprising SEQ ID NO: 394. 718. The composition of any one of embodiments 716-717, wherein the engineered guide RNA has a sequence of SEQ ID NO: 394. 719. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 720. The composition of embodiment 719, wherein the engineered guide RNA has at least 80%, 85%, 90%, 92%, 95%, 97%, or 99% sequence identity to a guide RNA comprising SEQ ID NO: 408. 721. The composition of any one of embodiments 719-720, wherein the engineered guide RNA has a sequence of SEQ ID NO: 408. 722. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 723. The composition of embodiment 722, wherein the engineered guide RNA has at least 80%, 85%, 90%, 92%, 95%, 97%, or 99% sequence identity to a guide RNA comprising SEQ ID NO: 482. 724. The composition of any one of embodiments 722-723, wherein the engineered guide RNA has a sequence of SEQ ID NO: 482. 725. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 726. The composition of embodiment 725, wherein the engineered guide RNA has at least 80%, 85%, 90%, 92%, 95%, 97%, or 99% sequence identity to a guide RNA comprising SEQ ID NO: 486. 727. The composition of any one of embodiments 725-726, wherein the engineered guide RNA has a sequence of SEQ ID NO: 486. 728. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 729. The composition of embodiment 728, wherein the engineered guide RNA has at least 80%, 85%, 90%, 92%, 95%, 97%, or 99% sequence identity to a guide RNA comprising SEQ ID NO: 487. 730. The composition of any one of embodiments 728-729, wherein the engineered guide RNA has a sequence of SEQ ID NO: 487. 731. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 732. The composition of embodiment 731, wherein the engineered guide RNA has at least 80%, 85%, 90%, 92%, 95%, 97%, or 99% sequence identity to a guide RNA comprising SEQ ID NO: 494. 733. The composition of any one of embodiments 731-732, wherein the engineered guide RNA has a sequence of SEQ ID NO: 494. 734. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 735. The composition of embodiment 734, wherein the engineered guide RNA has at least 80%, 85%, 90%, 92%, 95%, 97%, or 99% sequence identity to a guide RNA comprising SEQ ID NO: 502. 736. The composition of any one of embodiments 734-735, wherein the engineered guide RNA has a sequence of SEQ ID NO: 502. 737. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 738. The composition of embodiment 737, wherein the engineered guide RNA has at least 80%, 85%, 90%, 92%, 95%, 97%, or 99% sequence identity to a guide RNA comprising SEQ ID NO: 505. 739. The composition of any one of embodiments 737-738, wherein the engineered guide RNA has a sequence of SEQ ID NO: 505. 740. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 741. The composition of embodiment 740, wherein the engineered guide RNA has at least 80%, 85%, 90%, 92%, 95%, 97%, or 99% sequence identity to a guide RNA comprising SEQ ID NO: 512. 742. The composition of any one of embodiments 740-741, wherein the engineered guide RNA has a sequence of SEQ ID NO: 512. 743. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 744. The composition of embodiment 743, wherein the engineered guide RNA has at least 80%, 85%, 90%, 92%, 95%, 97%, or 99% sequence identity to a guide RNA comprising SEQ ID NO: 566. 745. The composition of any one of embodiments 743-744, wherein the engineered guide RNA has a sequence of SEQ ID NO: 566. 746. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 747. The composition of embodiment 746, wherein the engineered guide RNA has at least 80%, 85%, 90%, 92%, 95%, 97%, or 99% sequence identity to a guide RNA comprising SEQ ID NO: 593. 748. The composition of any one of embodiments 746-747, wherein the engineered guide RNA has a sequence of SEQ ID NO: 593. 749. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 750. The composition of embodiment 749, wherein the engineered guide RNA has at least 80%, 85%, 90%, 92%, 95%, 97%, or 99% sequence identity to a guide RNA comprising SEQ ID NO: 594. 751. The composition of any one of embodiments 749-750, wherein the engineered guide RNA has a sequence of SEQ ID NO: 594. 752. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 753. The composition of embodiment 752, wherein the engineered guide RNA has at least 80%, 85%, 90%, 92%, 95%, 97%, or 99% sequence identity to a guide RNA comprising SEQ ID NO: 606. 754. The composition of any one of embodiments 752-753, wherein the engineered guide RNA has a sequence of SEQ ID NO: 606. 755. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 756. The composition of embodiment 755, wherein the engineered guide RNA has at least 80%, 85%, 90%, 92%, 95%, 97%, or 99% sequence identity to a guide RNA comprising SEQ ID NO: 625. 757. The composition of any one of embodiments 755-756, wherein the engineered guide RNA has a sequence of SEQ ID NO: 625. 758. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 759. The composition of embodiment 758, wherein the engineered guide RNA has at least 80%, 85%, 90%, 92%, 95%, 97%, or 99% sequence identity to a guide RNA comprising SEQ ID NO: 635. 760. The composition of any one of embodiments 758-759, wherein the engineered guide RNA has a sequence of SEQ ID NO: 635. 761. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 762. The composition of embodiment 761, wherein the engineered guide RNA has at least 80%, 85%, 90%, 92%, 95%, 97%, or 99% sequence identity to a guide RNA comprising SEQ ID NO: 642. 763. The composition of any one of embodiments 761-762, wherein the engineered guide RNA has a sequence of SEQ ID NO: 642. 764. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 765. The composition of embodiment 764, wherein the engineered guide RNA has at least 80%, 85%, 90%, 92%, 95%, 97%, or 99% sequence identity to a guide RNA comprising SEQ ID NO: 679. 766. The composition of any one of embodiments 764-765, wherein the engineered guide RNA has a sequence of SEQ ID NO: 679. 767. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 768. The composition of embodiment 767, wherein the engineered guide RNA has at least 80%, 85%, 90%, 92%, 95%, 97%, or 99% sequence identity to a guide RNA comprising SEQ ID NO: 680. 769. The composition of any one of embodiments 767-768, wherein the engineered guide RNA has a sequence of SEQ ID NO: 680. 770. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 771. The composition of embodiment 770, wherein the engineered guide RNA has at least 80%, 85%, 90%, 92%, 95%, 97%, or 99% sequence identity to a guide RNA comprising SEQ ID NO: 694. 772. The composition of any one of embodiments 770-771, wherein the engineered guide RNA has a sequence of SEQ ID NO: 694. 773. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 774. The composition of embodiment 773, wherein the engineered guide RNA has at least 80%, 85%, 90%, 92%, 95%, 97%, or 99% sequence identity to a guide RNA comprising SEQ ID NO: 727. 775. The composition of any one of embodiments 773-774, wherein the engineered guide RNA has a sequence of SEQ ID NO: 727. 776. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 777. The composition of embodiment 776, wherein the engineered guide RNA has at least 80%, 85%, 90%, 92%, 95%, 97%, or 99% sequence identity to a guide RNA comprising SEQ ID NO: 737. 778. The composition of any one of embodiments 776-777, wherein the engineered guide RNA has a sequence of SEQ ID NO: 737. 779. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 780. The composition of embodiment 779, wherein the engineered guide RNA has at least 80%, 85%, 90%, 92%, 95%, 97%, or 99% sequence identity to a guide RNA comprising SEQ ID NO: 747. 781. The composition of any one of embodiments 779-780, wherein the engineered guide RNA has a sequence of SEQ ID NO: 747. 782. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 783. The composition of embodiment 782, wherein the engineered guide RNA has at least 80%, 85%, 90%, 92%, 95%, 97%, or 99% sequence identity to a guide RNA comprising SEQ ID NO: 748. 784. The composition of any one of embodiments 782-783, wherein the engineered guide RNA has a sequence of SEQ ID NO: 748. 785. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 786. The composition of embodiment 785, wherein the engineered guide RNA has at least 80%, 85%, 90%, 92%, 95%, 97%, or 99% sequence identity to a guide RNA comprising SEQ ID NO: 757. 787. The composition of any one of embodiments 785-786, wherein the engineered guide RNA has a sequence of SEQ ID NO: 757. 788. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 789. The composition of embodiment 788, wherein the engineered guide RNA has at least 80%, 85%, 90%, 92%, 95%, 97%, or 99% sequence identity to a guide RNA comprising SEQ ID NO: 769. 790. The composition of any one of embodiments 788-789, wherein the engineered guide RNA has a sequence of SEQ ID NO: 769. 791. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 792. The composition of embodiment 791, wherein the engineered guide RNA has at least 80%, 85%, 90%, 92%, 95%, 97%, or 99% sequence identity to a guide RNA comprising SEQ ID NO: 806. 793. The composition of any one of embodiments 791-792, wherein the engineered guide RNA has a sequence of SEQ ID NO: 806. 794. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 795. The composition of embodiment 794, wherein the engineered guide RNA has at least 80%, 85%, 90%, 92%, 95%, 97%, or 99% sequence identity to a guide RNA comprising SEQ ID NO: 810. 796. The composition of any one of embodiments 794-795, wherein the engineered guide RNA has a sequence of SEQ ID NO: 810. 797. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 798. The composition of embodiment 797, wherein the engineered guide RNA has at least 80%, 85%, 90%, 92%, 95%, 97%, or 99% sequence identity to a guide RNA comprising SEQ ID NO: 815. 799. The composition of any one of embodiments 797-798, wherein the engineered guide RNA has a sequence of SEQ ID NO: 815. 800. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 801. The composition of embodiment 800, wherein the engineered guide RNA has at least 80%, 85%, 90%, 92%, 95%, 97%, or 99% sequence identity to a guide RNA comprising SEQ ID NO: 851. 802. The composition of any one of embodiments 800-801, wherein the engineered guide RNA has a sequence of SEQ ID NO: 851. 803. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 804. The composition of embodiment 803, wherein the engineered guide RNA has at least 80%, 85%, 90%, 92%, 95%, 97%, or 99% sequence identity to a guide RNA comprising SEQ ID NO: 871. 805. The composition of any one of embodiments 803-804, wherein the engineered guide RNA has a sequence of SEQ ID NO: 871. 806. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 807. The composition of embodiment 806, wherein the engineered guide RNA has at least 80%, 85%, 90%, 92%, 95%, 97%, or 99% sequence identity to a guide RNA comprising SEQ ID NO: 873. 808. The composition of any one of embodiments 806-807, wherein the engineered guide RNA has a sequence of SEQ ID NO: 873. 809. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 810. The composition of embodiment 809, wherein the engineered guide RNA has at least 80%, 85%, 90%, 92%, 95%, 97%, or 99% sequence identity to a guide RNA comprising SEQ ID NO: 874. 811. The composition of any one of embodiments 809-810, wherein the engineered guide RNA has a sequence of SEQ ID NO: 874. 812. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 813. The composition of embodiment 812, wherein the engineered guide RNA has at least 80%, 85%, 90%, 92%, 95%, 97%, or 99% sequence identity to a guide RNA comprising SEQ ID NO: 880. 814. The composition of any one of embodiments 812-813, wherein the engineered guide RNA has a sequence of SEQ ID NO: 880. 815. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 816. The composition of embodiment 815, wherein the engineered guide RNA has at least 80%, 85%, 90%, 92%, 95%, 97%, or 99% sequence identity to a guide RNA comprising SEQ ID NO: 884. 817. The composition of any one of embodiments 815-816, wherein the engineered guide RNA has a sequence of SEQ ID NO: 884. 818. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 819. The composition of embodiment 818, wherein the engineered guide RNA has at least 80%, 85%, 90%, 92%, 95%, 97%, or 99% sequence identity to a guide RNA comprising SEQ ID NO: 892. 820. The composition of any one of embodiments 818-819, wherein the engineered guide RNA has a sequence of SEQ ID NO: 892. 821. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 822. The composition of embodiment 821, wherein the engineered guide RNA has at least 80%, 85%, 90%, 92%, 95%, 97%, or 99% sequence identity to a guide RNA comprising SEQ ID NO: 906. 823. The composition of any one of embodiments 821-822, wherein the engineered guide RNA has a sequence of SEQ ID NO: 906. 824. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 825. The composition of embodiment 824, wherein the engineered guide RNA has at least 80%, 85%, 90%, 92%, 95%, 97%, or 99% sequence identity to a guide RNA comprising SEQ ID NO: 930. 826. The composition of any one of embodiments 824-825, wherein the engineered guide RNA has a sequence of SEQ ID NO: 930. 827. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 828. The composition of embodiment 827, wherein the engineered guide RNA has at least 80%, 85%, 90%, 92%, 95%, 97%, or 99% sequence identity to a guide RNA comprising SEQ ID NO: 934. 829. The composition of any one of embodiments 827-828, wherein the engineered guide RNA has a sequence of SEQ ID NO: 934. 830. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 831. The composition of embodiment 830, wherein the engineered guide RNA has at least 80%, 85%, 90%, 92%, 95%, 97%, or 99% sequence identity to a guide RNA comprising SEQ ID NO: 935. 832. The composition of any one of embodiments 830-831, wherein the engineered guide RNA has a sequence of SEQ ID NO: 935. 833. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 834. The composition of embodiment 833, wherein the engineered guide RNA has at least 80%, 85%, 90%, 92%, 95%, 97%, or 99% sequence identity to a guide RNA comprising SEQ ID NO: 937. 835. The composition of any one of embodiments 833-834, wherein the engineered guide RNA has a sequence of SEQ ID NO: 937. 836. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 837. The composition of embodiment 836, wherein the engineered guide RNA has at least 80%, 85%, 90%, 92%, 95%, 97%, or 99% sequence identity to a guide RNA comprising SEQ ID NO: 944. 838. The composition of any one of embodiments 836-837, wherein the engineered guide RNA has a sequence of SEQ ID NO: 944. 839. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 840. The composition of embodiment 839, wherein the engineered guide RNA has at least 80%, 85%, 90%, 92%, 95%, 97%, or 99% sequence identity to a guide RNA comprising SEQ ID NO: 967. 841. The composition of any one of embodiments 839-840, wherein the engineered guide RNA has a sequence of SEQ ID NO: 967. 842. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 843. The composition of embodiment 842, wherein the engineered guide RNA has at least 80%, 85%, 90%, 92%, 95%, 97%, or 99% sequence identity to a guide RNA comprising SEQ ID NO: 976. 844. The composition of any one of embodiments 842-843, wherein the engineered guide RNA has a sequence of SEQ ID NO: 976. 845. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 846. The composition of embodiment 845, wherein the engineered guide RNA has at least 80%, 85%, 90%, 92%, 95%, 97%, or 99% sequence identity to a guide RNA comprising SEQ ID NO: 977. 847. The composition of any one of embodiments 845-846, wherein the engineered guide RNA has a sequence of SEQ ID NO: 977. 848. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 849. The composition of embodiment 848, wherein the engineered guide RNA has at least 80%, 85%, 90%, 92%, 95%, 97%, or 99% sequence identity to a guide RNA comprising SEQ ID NO: 985. 850. The composition of any one of embodiments 848-849, wherein the engineered guide RNA has a sequence of SEQ ID NO: 985. 851. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 852. The composition of embodiment 851, wherein the engineered guide RNA has at least 80%, 85%, 90%, 92%, 95%, 97%, or 99% sequence identity to a guide RNA comprising SEQ ID NO: 1002. 853. The composition of any one of embodiments 851-852, wherein the engineered guide RNA has a sequence of SEQ ID NO: 1002. 854. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 855. The composition of embodiment 854, wherein the engineered guide RNA has at least 80%, 85%, 90%, 92%, 95%, 97%, or 99% sequence identity to a guide RNA comprising SEQ ID NO: 1008. 856. The composition of any one of embodiments 854-855, wherein the engineered guide RNA has a sequence of SEQ ID NO: 1008. 857. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 858. The composition of embodiment 857, wherein the engineered guide RNA has at least 80%, 85%, 90%, 92%, 95%, 97%, or 99% sequence identity to a guide RNA comprising SEQ ID NO: 1051. 859. The composition of any one of embodiments 857-858, wherein the engineered guide RNA has a sequence of SEQ ID NO: 1051. 860. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 861. The composition of embodiment 860, wherein the engineered guide RNA has at least 80%, 85%, 90%, 92%, 95%, 97%, or 99% sequence identity to a guide RNA comprising SEQ ID NO: 1054. 862. The composition of any one of embodiments 860-861, wherein the engineered guide RNA has a sequence of SEQ ID NO: 1054. 863. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 864. The composition of embodiment 863, wherein the engineered guide RNA has at least 80%, 85%, 90%, 92%, 95%, 97%, or 99% sequence identity to a guide RNA comprising SEQ ID NO: 1058. 865. The composition of any one of embodiments 863-864, wherein the engineered guide RNA has a sequence of SEQ ID NO: 1058. 866. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 867. The composition of embodiment 866, wherein the engineered guide RNA has at least 80%, 85%, 90%, 92%, 95%, 97%, or 99% sequence identity to a guide RNA comprising SEQ ID NO: 1059. 868. The composition of any one of embodiments 866-867, wherein the engineered guide RNA has a sequence of SEQ ID NO: 1059. 869. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 870. The composition of embodiment 869, wherein the engineered guide RNA has at least 80%, 85%, 90%, 92%, 95%, 97%, or 99% sequence identity to a guide RNA comprising SEQ ID NO: 1066. 871. The composition of any one of embodiments 869-870, wherein the engineered guide RNA has a sequence of SEQ ID NO: 1066. 872. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 873. The composition of embodiment 872, wherein the engineered guide RNA has at least 80%, 85%, 90%, 92%, 95%, 97%, or 99% sequence identity to a guide RNA comprising SEQ ID NO: 1098. 874. The composition of any one of embodiments 872-873, wherein the engineered guide RNA has a sequence of SEQ ID NO: 1098. 875. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 876. The composition of embodiment 875, wherein the engineered guide RNA has at least 80%, 85%, 90%, 92%, 95%, 97%, or 99% sequence identity to a guide RNA comprising SEQ ID NO: 1103. 877. The composition of any one of embodiments 875-876, wherein the engineered guide RNA has a sequence of SEQ ID NO: 1103. 878. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 879. The composition of embodiment 878, wherein the engineered guide RNA has at least 80%, 85%, 90%, 92%, 95%, 97%, or 99% sequence identity to a guide RNA comprising SEQ ID NO: 1104. 880. The composition of any one of embodiments 878-879, wherein the engineered guide RNA has a sequence of SEQ ID NO: 1104. 881. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 882. The composition of embodiment 881, wherein the engineered guide RNA has at least 80%, 85%, 90%, 92%, 95%, 97%, or 99% sequence identity to a guide RNA comprising SEQ ID NO: 1116. 883. The composition of any one of embodiments 881-882, wherein the engineered guide RNA has a sequence of SEQ ID NO: 1116. 884. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 885. The composition of embodiment 884, wherein the engineered guide RNA has at least 80%, 85%, 90%, 92%, 95%, 97%, or 99% sequence identity to a guide RNA comprising SEQ ID NO: 1117. 886. The composition of any one of embodiments 884-885, wherein the engineered guide RNA has a sequence of SEQ ID NO: 1117. 887. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 888. The composition of embodiment 887, wherein the engineered guide RNA has at least 80%, 85%, 90%, 92%, 95%, 97%, or 99% sequence identity to a guide RNA comprising SEQ ID NO: 1163. 889. The composition of any one of embodiments 887-888, wherein the engineered guide RNA has a sequence of SEQ ID NO: 1163. 890. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 891. The composition of embodiment 890, wherein the engineered guide RNA has at least 80%, 85%, 90%, 92%, 95%, 97%, or 99% sequence identity to a guide RNA comprising SEQ ID NO: 1168. 892. The composition of any one of embodiments 890-891, wherein the engineered guide RNA has a sequence of SEQ ID NO: 1168. 893. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 894. The composition of embodiment 893, wherein the engineered guide RNA has at least 80%, 85%, 90%, 92%, 95%, 97%, or 99% sequence identity to a guide RNA comprising SEQ ID NO: 1183. 895. The composition of any one of embodiments 893-894, wherein the engineered guide RNA has a sequence of SEQ ID NO: 1183. 896. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 897. The composition of embodiment 896, wherein the engineered guide RNA has at least 80%, 85%, 90%, 92%, 95%, 97%, or 99% sequence identity to a guide RNA comprising SEQ ID NO: 1185. 898. The composition of any one of embodiments 896-897, wherein the engineered guide RNA has a sequence of SEQ ID NO: 1185. 899. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 900. The composition of embodiment 899, wherein the engineered guide RNA has at least 80%, 85%, 90%, 92%, 95%, 97%, or 99% sequence identity to a guide RNA comprising SEQ ID NO: 1193. 901. The composition of any one of embodiments 899-900, wherein the engineered guide RNA has a sequence of SEQ ID NO: 1193. 902. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 903. The composition of embodiment 902, wherein the engineered guide RNA has at least 80%, 85%, 90%, 92%, 95%, 97%, or 99% sequence identity to a guide RNA comprising SEQ ID NO: 1211. 904. The composition of any one of embodiments 902-903, wherein the engineered guide RNA has a sequence of SEQ ID NO: 1211. 905. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 906. The composition of embodiment 905, wherein the engineered guide RNA has at least 80%, 85%, 90%, 92%, 95%, 97%, or 99% sequence identity to a guide RNA comprising SEQ ID NO: 1212. 907. The composition of any one of embodiments 905-906, wherein the engineered guide RNA has a sequence of SEQ ID NO: 1212. 908. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 909. The composition of embodiment 908, wherein the engineered guide RNA has at least 80%, 85%, 90%, 92%, 95%, 97%, or 99% sequence identity to a guide RNA comprising SEQ ID NO: 1236. 910. The composition of any one of embodiments 908-909, wherein the engineered guide RNA has a sequence of SEQ ID NO: 1236. 911. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 912. The composition of embodiment 911, wherein the engineered guide RNA has at least 80%, 85%, 90%, 92%, 95%, 97%, or 99% sequence identity to a guide RNA comprising SEQ ID NO: 1293. 913. The composition of any one of embodiments 911-912, wherein the engineered guide RNA has a sequence of SEQ ID NO: 1293. 914. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 915. The composition of embodiment 914, wherein the engineered guide RNA has at least 80%, 85%, 90%, 92%, 95%, 97%, or 99% sequence identity to a guide RNA comprising SEQ ID NO: 1294. 916. The composition of any one of embodiments 914-915, wherein the engineered guide RNA has a sequence of SEQ ID NO: 1294. 917. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 918. The composition of embodiment 917, wherein the engineered guide RNA has at least 80%, 85%, 90%, 92%, 95%, 97%, or 99% sequence identity to a guide RNA comprising SEQ ID NO: 1296. 919. The composition of any one of embodiments 917-918, wherein the engineered guide RNA has a sequence of SEQ ID NO: 1296. 920. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 921. The composition of embodiment 920, wherein the engineered guide RNA has at least 80%, 85%, 90%, 92%, 95%, 97%, or 99% sequence identity to a guide RNA comprising SEQ ID NO: 1374. 922. The composition of any one of embodiments 920-921, wherein the engineered guide RNA has a sequence of SEQ ID NO: 1374. 923. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 924. The composition of embodiment 923, wherein the engineered guide RNA has at least 80%, 85%, 90%, 92%, 95%, 97%, or 99% sequence identity to a guide RNA comprising SEQ ID NO: 1391. 925. The composition of any one of embodiments 923-924, wherein the engineered guide RNA has a sequence of SEQ ID NO: 1391. 926. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 927. The composition of embodiment 926, wherein the engineered guide RNA has at least 80%, 85%, 90%, 92%, 95%, 97%, or 99% sequence identity to a guide RNA comprising SEQ ID NO: 1411. 928. The composition of any one of embodiments 926-927, wherein the engineered guide RNA has a sequence of SEQ ID NO: 1411. 929. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 930. The composition of embodiment 929, wherein the engineered guide RNA has at least 80%, 85%, 90%, 92%, 95%, 97%, or 99% sequence identity to a guide RNA comprising SEQ ID NO: 1463. 931. The composition of any one of embodiments 929-930, wherein the engineered guide RNA has a sequence of SEQ ID NO: 1463. 932. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 933. The composition of embodiment 932, wherein the engineered guide RNA has at least 80%, 85%, 90%, 92%, 95%, 97%, or 99% sequence identity to a guide RNA comprising SEQ ID NO: 1538. 934. The composition of any one of embodiments 932-933, wherein the engineered guide RNA has a sequence of SEQ ID NO: 1538. 935. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 936. The composition of embodiment 935, wherein the engineered guide RNA has at least 80%, 85%, 90%, 92%, 95%, 97%, or 99% sequence identity to a guide RNA comprising SEQ ID NO: 1539. 937. The composition of any one of embodiments 935-936, wherein the engineered guide RNA has a sequence of SEQ ID NO: 1539. 938. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 939. The composition of embodiment 938, wherein the engineered guide RNA has at least 80%, 85%, 90%, 92%, 95%, 97%, or 99% sequence identity to a guide RNA comprising SEQ ID NO: 1545. 940. The composition of any one of embodiments 938-939, wherein the engineered guide RNA has a sequence of SEQ ID NO: 1545. 941. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 942. The composition of embodiment 941, wherein the engineered guide RNA has at least 80%, 85%, 90%, 92%, 95%, 97%, or 99% sequence identity to a guide RNA comprising SEQ ID NO: 1552. 943. The composition of any one of embodiments 941-942, wherein the engineered guide RNA has a sequence of SEQ ID NO: 1552. 944. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 945. The composition of embodiment 944, wherein the engineered guide RNA has at least 80%, 85%, 90%, 92%, 95%, 97%, or 99% sequence identity to a guide RNA comprising SEQ ID NO: 1566. 946. The composition of any one of embodiments 944-945, wherein the engineered guide RNA has a sequence of SEQ ID NO: 1566. 947. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 948. The composition of embodiment 947, wherein the engineered guide RNA has at least 80%, 85%, 90%, 92%, 95%, 97%, or 99% sequence identity to a guide RNA comprising SEQ ID NO: 1567. 949. The composition of any one of embodiments 947-948, wherein the engineered guide RNA has a sequence of SEQ ID NO: 1567. 950. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 951. The composition of embodiment 950, wherein the engineered guide RNA has at least 80%, 85%, 90%, 92%, 95%, 97%, or 99% sequence identity to a guide RNA comprising SEQ ID NO: 1568. 952. The composition of any one of embodiments 950-951, wherein the engineered guide RNA has a sequence of SEQ ID NO: 1568. 953. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 954. The composition of embodiment 953, wherein the engineered guide RNA has at least 80%, 85%, 90%, 92%, 95%, 97%, or 99% sequence identity to a guide RNA comprising SEQ ID NO: 1569. 955. The composition of any one of embodiments 953-954, wherein the engineered guide RNA has a sequence of SEQ ID NO: 1569. 956. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 957. The composition of embodiment 956, wherein the engineered guide RNA has at least 80%, 85%, 90%, 92%, 95%, 97%, or 99% sequence identity to a guide RNA comprising SEQ ID NO: 1570. 958. The composition of any one of embodiments 956-957, wherein the engineered guide RNA has a sequence of SEQ ID NO: 1570. 959. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 960. The composition of embodiment 959, wherein the engineered guide RNA has at least 80%, 85%, 90%, 92%, 95%, 97%, or 99% sequence identity to a guide RNA comprising SEQ ID NO: 1571. 961. The composition of any one of embodiments 959-960, wherein the engineered guide RNA has a sequence of SEQ ID NO: 1571. 962. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 963. The composition of embodiment 962, wherein the engineered guide RNA has at least 80%, 85%, 90%, 92%, 95%, 97%, or 99% sequence identity to a guide RNA comprising SEQ ID NO: 1572. 964. The composition of any one of embodiments 962-963, wherein the engineered guide RNA has a sequence of SEQ ID NO: 1572. 965. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 966. The composition of embodiment 965, wherein the engineered guide RNA has at least 80%, 85%, 90%, 92%, 95%, 97%, or 99% sequence identity to a guide RNA comprising SEQ ID NO: 1573. 967. The composition of any one of embodiments 965-966, wherein the engineered guide RNA has a sequence of SEQ ID NO: 1573. 968. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 969. The composition of embodiment 968, wherein the engineered guide RNA has at least 80%, 85%, 90%, 92%, 95%, 97%, or 99% sequence identity to a guide RNA comprising SEQ ID NO: 1574. 970. The composition of any one of embodiments 968-969, wherein the engineered guide RNA has a sequence of SEQ ID NO: 1574. 971. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 972. The composition of embodiment 971, wherein the engineered guide RNA has at least 80%, 85%, 90%, 92%, 95%, 97%, or 99% sequence identity to a guide RNA comprising SEQ ID NO: 1575. 973. The composition of any one of embodiments 971-972, wherein the engineered guide RNA has a sequence of SEQ ID NO: 1575. 974. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 975. The composition of embodiment 974, wherein the engineered guide RNA has at least 80%, 85%, 90%, 92%, 95%, 97%, or 99% sequence identity to a guide RNA comprising SEQ ID NO: 1576. 976. The composition of any one of embodiments 974-975, wherein the engineered guide RNA has a sequence of SEQ ID NO: 1576. 977. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 978. The composition of embodiment 977, wherein the engineered guide RNA has at least 80%, 85%, 90%, 92%, 95%, 97%, or 99% sequence identity to a guide RNA comprising SEQ ID NO: 1577. 979. The composition of any one of embodiments 977-978, wherein the engineered guide RNA has a sequence of SEQ ID NO: 1577. 980. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 981. The composition of embodiment 980, wherein the engineered guide RNA has at least 80%, 85%, 90%, 92%, 95%, 97%, or 99% sequence identity to a guide RNA comprising SEQ ID NO: 1578. 982. The composition of any one of embodiments 980-981, wherein the engineered guide RNA has a sequence of SEQ ID NO: 1578. 983. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 984. The composition of embodiment 983, wherein the engineered guide RNA has at least 80%, 85%, 90%, 92%, 95%, 97%, or 99% sequence identity to a guide RNA comprising SEQ ID NO: 1579. 985. The composition of any one of embodiments 983-984, wherein the engineered guide RNA has a sequence of SEQ ID NO: 1579. 986. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 987. The composition of embodiment 986, wherein the engineered guide RNA has at least 80%, 85%, 90%, 92%, 95%, 97%, or 99% sequence identity to a guide RNA comprising SEQ ID NO: 1580. 988. The composition of any one of embodiments 986-987, wherein the engineered guide RNA has a sequence of SEQ ID NO: 1580. 989. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 990. The composition of embodiment 989, wherein the engineered guide RNA has at least 80%, 85%, 90%, 92%, 95%, 97%, or 99% sequence identity to a guide RNA comprising SEQ ID NO: 1581. 991. The composition of any one of embodiments 989-990, wherein the engineered guide RNA has a sequence of SEQ ID NO: 1581. 992. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 993. The composition of embodiment 992, wherein the engineered guide RNA has at least 80%, 85%, 90%, 92%, 95%, 97%, or 99% sequence identity to a guide RNA comprising SEQ ID NO: 1582. 994. The composition of any one of embodiments 992-993, wherein the engineered guide RNA has a sequence of SEQ ID NO: 1582. 995. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 996. The composition of embodiment 995, wherein the engineered guide RNA has at least 80%, 85%, 90%, 92%, 95%, 97%, or 99% sequence identity to a guide RNA comprising SEQ ID NO: 1583. 997. The composition of any one of embodiments 995-996, wherein the engineered guide RNA has a sequence of SEQ ID NO: 1583. 998. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 999. The composition of embodiment 998, wherein the engineered guide RNA has at least 80%, 85%, 90%, 92%, 95%, 97%, or 99% sequence identity to a guide RNA comprising SEQ ID NO: 1584. 1000. The composition of any one of embodiments 998-999, wherein the engineered guide RNA has a sequence of SEQ ID NO: 1584. 1001. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 1002. The composition of embodiment 1001, wherein the engineered guide RNA has at least 80%, 85%, 90%, 92%, 95%, 97%, or 99% sequence identity to a guide RNA comprising SEQ ID NO: 1585. 1003. The composition of any one of embodiments 1001-1002, wherein the engineered guide RNA has a sequence of SEQ ID NO: 1585. 1004. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 1005. The composition of embodiment 1004, wherein the engineered guide RNA has at least 80%, 85%, 90%, 92%, 95%, 97%, or 99% sequence identity to a guide RNA comprising SEQ ID NO: 1586. 1006. The composition of any one of embodiments 1004-1005, wherein the engineered guide RNA has a sequence of SEQ ID NO: 1586. 1007. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 1008. The composition of embodiment 1007, wherein the engineered guide RNA has at least 80%, 85%, 90%, 92%, 95%, 97%, or 99% sequence identity to a guide RNA comprising SEQ ID NO: 1587. 1009. The composition of any one of embodiments 1007-1008, wherein the engineered guide RNA has a sequence of SEQ ID NO: 1587. 1010. The composition of embodiment 7, wherein the one or more structural features comprise a 1 nucleotide mismatch formed 3 nucleotides downstream (3′) from the target A, and a 6 nucleotide internal symmetric loop formed 20 nucleotides downstream (3′) from the target A. 1011. The composition of embodiment 1010, wherein the engineered guide RNA has at least 80%, 85%, 90%, 92%, 95%, 97%, or 99% sequence identity to a guide RNA comprising SEQ ID NO: 1588. 1012. The composition of any one of embodiments 1010-1011, wherein the engineered guide RNA has a sequence of SEQ ID NO: 1588. 1013. The composition of any one of embodiments 6-8, wherein the one or more structural features comprise: a) a first 6/6 symmetric internal loop, and b) at least one additional structural feature selected from the group consisting of: a second 6/6 symmetric internal loop, a 5/5 symmetric internal loop, a 4/4 symmetric bulge, a 3/3 symmetric bulge, and a 2/2 symmetric bulge. 1014. The composition of embodiment 1013, wherein the guide-target RNA scaffold further comprises an A/C mismatch, wherein the cytosine of the A/C mismatch is present in the engineered guide RNA opposite the one or more target adenosines; and wherein the one or more structural features comprise: a) the first 6/6 symmetric internal loop positioned from position −4 to −8, relative to the A/C mismatch; b) the second 6/6 symmetric internal loop positioned from position+31 to +35, relative to the A/C mismatch. 1015. The composition of embodiment 1014, wherein the guide-target RNA scaffold further comprises an A/C mismatch, wherein the cytosine of the A/C mismatch is present in the engineered guide RNA opposite the one or more target adenosines; and wherein the one or more structural features comprise: a) the first 6/6 symmetric internal loop at position −6, relative to the A/C mismatch; b) the second 6/6 symmetric internal loop at position+33, relative to the A/C mismatch. 1016. The composition of embodiment 1014 or 1015, wherein the first 6/6 symmetric internal loop comprises the sequence GGAACU on the engineered guide RNA side, and the sequence UUCAGA on the target RNA side. 1017. The composition of embodiment 1014 or 1015, wherein the second 6/6 symmetric internal loop comprises the sequence CUGACC on the engineered guide RNA side, and the sequence AGAUUU on the target RNA side. 1018. The composition of any one of embodiments 6-8, wherein the one or more structural features comprise a first 6/6 symmetric internal loop and a second 6/6 symmetric internal loop and wherein each A in the target RNA is base paired to a U in the engineered guide RNA. 1019. The composition of any one of embodiments 6-8, wherein the engineered guide RNA comprises a sequence of any one of SEQ ID NO: 1566, SEQ ID NO: 1567, SEQ ID NO: 1569, SEQ ID NO: 1570, SEQ ID NO: 1571, SEQ ID NO: 1572, SEQ ID NO: 1573, SEQ ID NO: 1575, SEQ ID NO: 1577, SEQ ID NO: 1581, SEQ ID NO: 1585, SEQ ID NO: 1587, or SEQ ID NO: 1588. 1020. The composition of any one of embodiments 6-8, wherein the engineered guide RNA comprises a sequence of any one of SEQ ID NO: 1575, SEQ ID NO: 593, SEQ ID NO: 1573, SEQ ID NO: 934, SEQ ID NO: 1569, SEQ ID NO: 1567, SEQ ID NO: 851, SEQ ID NO: 1211, SEQ ID NO: 1571, SEQ ID NO: 937, SEQ ID NO: 1574, SEQ ID NO: 1570, SEQ ID NO: 1566, SEQ ID NO: 1117, SEQ ID NO: 906, SEQ ID NO: 1572, SEQ ID NO: 1104, SEQ ID NO: 352, SEQ ID NO: 512, SEQ ID NO: 1587, SEQ ID NO: 375, SEQ ID NO: 1588, SEQ ID NO: 977, SEQ ID NO: 642, SEQ ID NO: 1236, SEQ ID NO: 1584, SEQ ID NO: 252, SEQ ID NO: 394, SEQ ID NO: 482, SEQ ID NO: 1585, SEQ ID NO: 291, SEQ ID NO: 356, SEQ ID NO: 1054, SEQ ID NO: 1581, SEQ ID NO: 1103, SEQ ID NO: 502, SEQ ID NO: 769, SEQ ID NO: 408, SEQ ID NO: 1586, SEQ ID NO: 1008, SEQ ID NO: 737, SEQ ID NO: 985, SEQ ID NO: 679, SEQ ID NO: 727, SEQ ID NO: 1578, SEQ ID NO: 365, SEQ ID NO: 1580, SEQ ID NO: 487, SEQ ID NO: 1098, or SEQ ID NO: 976. 1021. The composition of embodiment 1020, wherein the engineered guide RNA comprises the sequence of SEQ ID NO: 1575. 1022. The composition of embodiment 1020, wherein the engineered guide RNA comprises the sequence of SEQ ID NO: 593. 1023. The composition of embodiment 1020, wherein the engineered guide RNA comprises the sequence of SEQ ID NO: 1573. 1024. The composition of embodiment 1020, wherein the engineered guide RNA comprises the sequence of SEQ ID NO: 934. 1025. The composition of embodiment 1020, wherein the engineered guide RNA comprises the sequence of SEQ ID NO: 1569. 1026. The composition of embodiment 1020, wherein the engineered guide RNA comprises the sequence of SEQ ID NO: 1567. 1027. The composition of embodiment 1020, wherein the engineered guide RNA comprises the sequence of SEQ ID NO: 851. 1028. The composition of embodiment 1020, wherein the engineered guide RNA comprises the sequence of SEQ ID NO: 1211. 1029. The composition of embodiment 1020, wherein the engineered guide RNA comprises the sequence of SEQ ID NO: 1571. 1030. The composition of embodiment 1020, wherein the engineered guide RNA comprises the sequence of SEQ ID NO: 937. 1031. The composition of any one of embodiments 6-8, wherein the engineered guide RNA comprises a sequence of any one of SEQ ID NO: 1573, SEQ ID NO: 1588, SEQ ID NO: 1545, SEQ ID NO: 1575, SEQ ID NO: 1569, SEQ ID NO: 1584, SEQ ID NO: 1572, SEQ ID NO: 1567, SEQ ID NO: 1570, SEQ ID NO: 1587, SEQ ID NO: 1574, SEQ ID NO: 625, SEQ ID NO: 1571, SEQ ID NO: 874, SEQ ID NO: 17, SEQ ID NO: 1585, SEQ ID NO: 757, SEQ ID NO: 1581, SEQ ID NO: 1538, SEQ ID NO: 8, SEQ ID NO: 1002, SEQ ID NO: 1566, SEQ ID NO: 486, SEQ ID NO: 1552, SEQ ID NO: 505, SEQ ID NO: 635, SEQ ID NO: 606, SEQ ID NO: 884, SEQ ID NO: 1054, SEQ ID NO: 880, SEQ ID NO: 1411, SEQ ID NO: 1568, SEQ ID NO: 871, SEQ ID NO: 1580, SEQ ID NO: 1539, SEQ ID NO: 14, SEQ ID NO: 892, SEQ ID NO: 1116, SEQ ID NO: 15, SEQ ID NO: 1586, SEQ ID NO: 593, SEQ ID NO: 10, SEQ ID NO: 977, SEQ ID NO: 1578, SEQ ID NO: 1579, SEQ ID NO: 747, SEQ ID NO: 1577, 748, SEQ ID NO: 873, or SEQ ID NO: 494. 1032. The composition of embodiment 1031, wherein the engineered guide RNA comprises the sequence of SEQ ID NO: 1573. 1033. The composition of embodiment 1031, wherein the engineered guide RNA comprises the sequence of SEQ ID NO: 1588. 1034. The composition of embodiment 1031, wherein the engineered guide RNA comprises the sequence of SEQ ID NO: 1545. 1035. The composition of embodiment 1031, wherein the engineered guide RNA comprises the sequence of SEQ ID NO: 1575. 1036. The composition of embodiment 1031, wherein the engineered guide RNA comprises the sequence of SEQ ID NO: 1569. 1037. The composition of embodiment 1031, wherein the engineered guide RNA comprises the sequence of SEQ ID NO: 1584. 1038. The composition of embodiment 1031, wherein the engineered guide RNA comprises the sequence of SEQ ID NO: 1572. 1039. The composition of embodiment 1031, wherein the engineered guide RNA comprises the sequence of SEQ ID NO: 1567. 1040. The composition of embodiment 1031, wherein the engineered guide RNA comprises the sequence of SEQ ID NO: 1570. 1041. The composition of embodiment 1031, wherein the engineered guide RNA comprises the sequence of SEQ ID NO: 1587. 1042. The composition of any one of embodiments 6-8, wherein the engineered guide RNA comprises a sequence of any one of SEQ ID NO: 1575, SEQ ID NO: 1573, SEQ ID NO: 1567, SEQ ID NO: 1569, SEQ ID NO: 1570, SEQ ID NO: 1566, SEQ ID NO: 1572, SEQ ID NO: 1587, SEQ ID NO: 1571, SEQ ID NO: 1574, SEQ ID NO: 1584, SEQ ID NO: 1588, SEQ ID NO: 1054, SEQ ID NO: 1586, SEQ ID NO: 1585, SEQ ID NO: 1581, SEQ ID NO: 1578, SEQ ID NO: 1580, SEQ ID NO: 934, SEQ ID NO: 72, SEQ ID NO: 1582, SEQ ID NO: 1066, SEQ ID NO: 1183, SEQ ID NO: 1577, SEQ ID NO: 967, SEQ ID NO: 1568, SEQ ID NO: 930, SEQ ID NO: 566, SEQ ID NO: 1463, SEQ ID NO: 1294, SEQ ID NO: 1293, SEQ ID NO: 1391, SEQ ID NO: 1579, SEQ ID NO: 1583, SEQ ID NO: 944, SEQ ID NO: 815, SEQ ID NO: 1168, SEQ ID NO: 593, SEQ ID NO: 594, SEQ ID NO: 694, SEQ ID NO: 1576, SEQ ID NO: 1193, SEQ ID NO: 1051, SEQ ID NO: 1212, SEQ ID NO: 806, SEQ ID NO: 1059, SEQ ID NO: 1374, SEQ ID NO: 195, SEQ ID NO: 358, SEQ ID NO: or SEQ ID NO: 1296. 1043. The composition of embodiment 1042, wherein the engineered guide RNA comprises the sequence of SEQ ID NO: 1575. 1044. The composition of embodiment 1042, wherein the engineered guide RNA comprises the sequence of SEQ ID NO: 1573. 1045. The composition of embodiment 1042, wherein the engineered guide RNA comprises the sequence of SEQ ID NO: 1567. 1046. The composition of embodiment 1042, wherein the engineered guide RNA comprises the sequence of SEQ ID NO: 1569. 1047. The composition of embodiment 1042, wherein the engineered guide RNA comprises the sequence of SEQ ID NO: 1570. 1048. The composition of embodiment 1042, wherein the engineered guide RNA comprises the sequence of SEQ ID NO: 1566. 1049. The composition of embodiment 1042, wherein the engineered guide RNA comprises the sequence of SEQ ID NO: 1572. 1050. The composition of embodiment 1042, wherein the engineered guide RNA comprises the sequence of SEQ ID NO: 1587. 1051. The composition of embodiment 1042, wherein the engineered guide RNA comprises the sequence of SEQ ID NO: 1571. 1052. The composition of embodiment 1042, wherein the engineered guide RNA comprises the sequence of SEQ ID NO: 1574. 1053. The composition of any one of embodiments 6-8, wherein the engineered guide RNA comprises a sequence of any one of SEQ ID NO: 1575, SEQ ID NO: 1573, SEQ ID NO: 1569, SEQ ID NO: 1574, SEQ ID NO: 1570, SEQ ID NO: 1572, SEQ ID NO: 1567, SEQ ID NO: 1587, SEQ ID NO: 1566, SEQ ID NO: 1571, SEQ ID NO: 1588, SEQ ID NO: 72, SEQ ID NO: 1586, SEQ ID NO: 1584, SEQ ID NO: 1581, SEQ ID NO: 1578, SEQ ID NO: 1585, SEQ ID NO: 1582, SEQ ID NO: 1580, SEQ ID NO: 1183, SEQ ID NO: 1568, SEQ ID NO: 1066, SEQ ID NO: 1391, SEQ ID NO: 1168, SEQ ID NO: 1293, SEQ ID NO: 1577, SEQ ID NO: 1054, SEQ ID NO: 566, SEQ ID NO: 1579, SEQ ID NO: 930, SEQ ID NO: 694, SEQ ID NO: 944, SEQ ID NO: 195, SEQ ID NO: 1583, SEQ ID NO: 815, SEQ ID NO: 1576, SEQ ID NO: 1051, SEQ ID NO: 1411, SEQ ID NO: 24, SEQ ID NO: 1163, SEQ ID NO: 935, SEQ ID NO: 680, SEQ ID NO: 1212, SEQ ID NO: 594, SEQ ID NO: 1185, SEQ ID NO: 1463, SEQ ID NO: 1058, SEQ ID NO: 810, SEQ ID NO: 392, SEQ ID NO: or SEQ ID NO: 1104. 1054. The composition of embodiment 1053, wherein the engineered guide RNA comprises the sequence of SEQ ID NO: 1575. 1055. The composition of embodiment 1053, wherein the engineered guide RNA comprises the sequence of SEQ ID NO: 1573. 1056. The composition of embodiment 1053, wherein the engineered guide RNA comprises the sequence of SEQ ID NO: 1569. 1057. The composition of embodiment 1053, wherein the engineered guide RNA comprises the sequence of SEQ ID NO: 1574. 1058. The composition of embodiment 1053, wherein the engineered guide RNA comprises the sequence of SEQ ID NO: 1570. 1059. The composition of embodiment 1053, wherein the engineered guide RNA comprises the sequence of SEQ ID NO: 1572. 1060. The composition of embodiment 1053, wherein the engineered guide RNA comprises the sequence of SEQ ID NO: 1567. 1061. The composition of embodiment 1053, wherein the engineered guide RNA comprises the sequence of SEQ ID NO: 1587. 1062. The composition of embodiment 1053, wherein the engineered guide RNA comprises the sequence of SEQ ID NO: 1566. 1063. The composition of embodiment 1053, wherein the engineered guide RNA comprises the sequence of SEQ ID NO: 1571. 1064. The composition of embodiment 1, wherein the one or more structural features comprises the bulge, wherein the bulge is a symmetric bulge. 1065. The composition of embodiment 1, wherein the one or more structural features comprises the bulge, wherein the bulge is an asymmetric bulge. 1066. The composition of embodiment 1, wherein the one or more structural features comprises the internal loop, wherein the internal loop is a symmetric internal loop. 1067. The composition of embodiment 1, wherein the one or more structural features comprises the internal loop, wherein the internal loop is an asymmetric internal loop. 1068. The composition of embodiment 1, wherein the guide-target RNA scaffold further comprises a Wobble base pair. 1069. The composition of embodiment 1, wherein the one or more structural features comprises the hairpin, wherein the hairpin is a recruitment hairpin or a non-recruitment hairpin. 1070. The composition of embodiment 1, wherein the one or more structural features comprises the mismatch formed by a base in the engineered guide RNA to a G, a C, or a U in the DUX4 target RNA. 1071. The composition of embodiment 1, wherein the RNA editing entity comprises ADAR1, ADAR2, ADAR3, or any combination thereof. 1072. The composition of embodiment 1, wherein the RNA editing of one or more target adenosines comprises hyper-editing. 1073. The composition of embodiment 1072, wherein the hyper-editing comprises editing of more than one A in the polyA signal sequence of the DUX4 target RNA. 1074. The composition of embodiment 1, wherein the internal loop of the engineered guide RNA comprises any nucleotide in any positional order, wherein the nucleotide in any positional order is not complementary to their positional counterpart in the DUX 4 target RNA. 1075. The composition of any one of embodiments 1-1074, wherein the engineered guide RNA or the engineered polynucleotide encoding the engineered guide RNA is circular. 1076. The composition of any one of embodiments 1-1075, wherein the engineered guide RNA or the engineered polynucleotide encoding the engineered guide RNA comprises a U7 hairpin sequence, a SmOPT sequence, or a combination thereof and optionally wherein the U7 hairpin sequence comprises SEQ ID NO 1591 or 1593 and wherein the SmOPT sequence comprises SEQ ID NO: 1595. 1077. The composition of embodiment 1, wherein the DUX4 target RNA comprises a pre-mRNA transcript of DUX4. 1078. The composition of embodiment 1077, wherein at least 40%, at least 50%, at least 60%, at least 70%, at least 80%, at least 85%, at least 90%, at least 95%, or at least 99% of the pre-mRNA transcripts of DUX4 have at least one edit in the polyA signal sequence. 1079. The composition of embodiment 1078, wherein at least 80% of the pre-mRNA transcripts of DUX4 have at least one edit in the polyA signal sequence. 1080. The composition of any one of embodiments 1-1079, wherein the editing of one or more adenosines facilitates a mRNA knockdown. 1081. The composition of embodiment 1080, wherein the mRNA knockdown comprises a knockdown of DUX4 mRNA. 1082. The composition of embodiment 1080 or 1081, wherein the mRNA knockdown comprises a mRNA knockdown of a protein downstream of DUX4, wherein the protein downstream of DUX4 comprises SLC34A2, LEUTX, ZSCAN4, PRAMEF12, TRIM43, DEFB103, or MBD3L2, or any combination thereof 1083. The method of any one of embodiments 1080-1082, wherein the mRNA knockdown comprises a reduction of at least 40%, at least 50%, at least 60%, at least 70%, at least 80%, at least 85%, at least 90%, at least 95%, or at least 99% of a mRNA level after RNA editing as compared to a mRNA level before RNA editing. 1084. The composition of embodiment 1083, wherein the mRNA knockdown is at least 50% of the mRNA level as compared to the mRNA level before RNA editing. 1085. The composition of embodiment 1083, wherein the mRNA knockdown is at least 70% of the mRNA level as compared to the mRNA level before RNA editing. 1086. The composition of any one of embodiments 1-1085, wherein the editing of one or more adenosines facilitates a protein knockdown. 1087. The composition of embodiment 1086, wherein the protein knockdown comprises a knockdown of DUX4. 1088. The composition of embodiment 1086 or 1087, wherein the protein knockdown comprises a knockdown of a protein downstream of DUX4, wherein the protein downstream of DUX4 comprises SLC34A2, LEUTX, ZSCAN4, PRAMEF12, TRIM43, DEFB103, or MBD3L2, or any combination thereof 1089. The composition of any one of embodiments 1086-1088, wherein the protein knockdown comprises a reduction of at least 40%, at least 50%, at least 60%, at least 70%, at least 80%, at least 85%, at least 90%, at least 95%, or at least 99% of the protein level after RNA editing as compared to the protein level before RNA editing. 1090. The composition of any one of embodiments 1086-1088, wherein the protein knockdown comprises a reduction of at least 40%, at least 50%, at least 60%, at least 70%, at least 80%, at least 85%, at least 90%, at least 95%, or at least 99% of the protein level in an ADAR expressing cell as compared to a cell comprising an nonfunctional ADAR gene. 1091. The composition of any one of embodiments 1086-1090, wherein the protein knockdown comprises ADAR-dependent protein knockdown. 1092. The composition of embodiment 1091, wherein the ADAR-dependent protein knockdown comprises a reduction of at least 50%, at least 60%, at least 70%, at least 80%, at least 85%, at least 90%, at least 95%, or at least 99% of the protein level as compared to the protein level before RNA editing. 1093. The composition of any one of embodiments 1-1092, wherein the engineered guide RNA is an in vitro transcribed (IVT) engineered guide RNA. 1094. The composition of any one of embodiments 1-1092, comprising the engineered polynucleotide. 1095. The composition of embodiment 1094, wherein the engineered polynucleotide is comprised in or on a vector. 1096. The composition of embodiment 1095, wherein the vector is a viral vector, and wherein the engineered polynucleotide is encapsidated in the viral vector. 1097. The composition of embodiment 1096, wherein the viral vector is an adeno-associated viral (AAV) vector or a derivative thereof. 1098. The composition of embodiment 1097, wherein the AAV vector is an AAV1 serotype, an AAV2 serotype, an AAV3 serotype, an AAV4 serotype, an AAV5 serotype, an AAV6 serotype, an AAV7 serotype, an AAV8 serotype, an AAV9 serotype, an AAV10 serotype, an AAV 11 serotype, an AAV12 serotype, an AAV13 serotype, an AAV14 serotype, an AAV15 serotype, an AAV16 serotype, an AAV.rh8 serotype, an AAV.rh10 serotype, an AAV.rh20 serotype, an AAV.rh39 serotype, an AAV.Rh74 serotype, an AAV.RHM4-1 serotype, an AAV.hu37 serotype, an AAV.Anc80 serotype, an AAV.Anc80L65 serotype, an AAV.7m8 serotype, an AAV.PHP.B serotype, an AAV2.5 serotype, an AAV2tYF serotype, an AAV3B serotype, an AAV.LK03 serotype, an AAV.HSC1 serotype, an AAV.HSC2 serotype, an AAV.HSC3 serotype, an AAV.HSC4 serotype, an AAV.HSC5 serotype, an AAV.HSC6 serotype, an AAV.HSC7 serotype, an AAV.HSC8 serotype, an AAV.HSC9 serotype, an AAV.HSC10 serotype, an AAV.HSC11 serotype, an AAV.HSC12 serotype, an AAV.HSC13 serotype, an AAV.HSC14 serotype, an AAV.HSC15 serotype, an AAV.HSC16 serotype, and an AAVhu68 serotype, a derivative of any of these serotypes, or any combination thereof. 1099. The composition of embodiment 1098, wherein the AAV vector is an AAV5 serotype, an AAV6 serotype, an AAV8 serotype, or an AAV9 serotype. 1100. The composition of any one of embodiments 1097-1099, wherein the AAV vector is a recombinant AAV (rAAV) vector, a hybrid AAV vector, a chimeric AAV vector, a self-complementary AAV (scAAV) vector, or any combination thereof. 1101. The composition of embodiment 1095, wherein the vector is a non-viral vector. 1102. The composition of embodiment 1101, wherein the non-viral vector is a lipid nanoparticle (LNP), a liposome, or a polymer nanoparticle. 1103. The composition of embodiment 1094, wherein the engineered polynucleotide is a DNA polynucleotide encoding the engineered guide RNA. 1104. The composition of embodiment 1, wherein the engineered guide RNA comprises at least 80%, 85%, 90%, 92%, 95%, 97%, or 99% sequence identity to any one of SEQ ID NO: 2-SEQ ID NO: 1589. 1105. The composition of embodiment 1, wherein the engineered guide RNA comprises a sequence of any one of SEQ ID NO: 2-SEQ ID NO: 1589. 1106. A pharmaceutical composition comprising: a) the composition of any one of embodiments 1-1105; and b) a pharmaceutically acceptable: excipient, carrier, or diluent. 1107. A method of treating a disease or a condition in a subject in need thereof, the method comprising administering to the subject a therapeutically effective amount of the composition of any one of embodiments 1-1105 or the pharmaceutical composition of embodiment 1106. 1108. The method of embodiment 1107, wherein the disease or condition comprises facioscapulohumeral muscular dystrophy. 1109. The method of embodiment 1108, wherein FSHD comprises Type I FSHD. 1110. The method of embodiment 1108, wherein FSHD comprises Type II FSHD. 1111. The method of any one of embodiments 1107-1110, wherein the administering comprises parenteral administration, intravenous administration, subcutaneous administration, intrathecal administration, intraperitoneal administration, intramuscular administration, intravascular administration, infusion administration, topical administration, oral administration, inhalation administration, intraduodenal administration, rectal administration, or a combination thereof. 1112. The method of embodiment 1111, comprising the administering, wherein the administration is oral administration. 1113. The method of embodiment 1111, comprising the administering, wherein the administration is in the form of an injection. 1114. The method of any one of embodiments 1107-1113, wherein the administering comprises systemic administration. 1115. A method of editing a DUX4 RNA the method comprising contacting the DUX4 RNA with any one of the compositions of embodiments 1-1105 and an RNA editing entity, thereby editing the DUX4 RNA. 1116. The method of embodiment 1115, wherein the editing comprises editing at any A position of a polyA tail of the DUX4 RNA. 1117. The method of embodiment 1116, wherein the editing comprises editing from about 44% to about 91% of any A position of the polyA tail of the DUX4 RNA as measured in an in vitro assay. 1118. The method of embodiment 1116, wherein the editing comprises editing at position 0 of the polyA tail of the DUX4 RNA. 1119. The method of embodiment 1118, wherein the editing comprises editing from about 50% to about 66% of the A at position 0 in the polyA tail of the DUX4 RNA as measured in an in vitro assay. 1120. The method of embodiment 1118 or 1119, wherein the engineered guide RNA in the composition comprises a sequence selected from the group consisting of: SEQ ID NO: 1575, SEQ ID NO: 593, SEQ ID NO: 1573, SEQ ID NO: 934, SEQ ID NO: 1569, SEQ ID NO: 1567, SEQ ID NO: 851, SEQ ID NO: 1211, SEQ ID NO: 1571, SEQ ID NO: 937, SEQ ID NO: 1574, SEQ ID NO: 1570, SEQ ID NO: 1566, SEQ ID NO: 1117, SEQ ID NO: 906, SEQ ID NO: 1572, SEQ ID NO: 1104, SEQ ID NO: 352, SEQ ID NO: 512, SEQ ID NO: 1587, SEQ ID NO: 375, SEQ ID NO: 1588, SEQ ID NO: 977, SEQ ID NO: 642, SEQ ID NO: 1236, SEQ ID NO: 1584, SEQ ID NO: 252, SEQ ID NO: 394, SEQ ID NO: 482, SEQ ID NO: 1585, SEQ ID NO: 291, SEQ ID NO: 356, SEQ ID NO: 1054, SEQ ID NO: 1581, SEQ ID NO: 1103, SEQ ID NO: 502, SEQ ID NO: 769, SEQ ID NO: 408, SEQ ID NO: 1586, SEQ ID NO: 1008, SEQ ID NO: 737, SEQ ID NO: 985, SEQ ID NO: 679, SEQ ID NO: 727, SEQ ID NO: 1578, SEQ ID NO: 365, SEQ ID NO: 1580, SEQ ID NO: 487, SEQ ID NO: 1098, and SEQ ID NO: 976. 1121. The method of embodiment 1116, wherein the editing comprises editing at position 3 of the polyA tail of the DUX4 RNA. 1122. The method of embodiment 1121, wherein the editing comprises editing from about 76% to about 91% of the A at position 3 in the polyA tail of the DUX4 RNA as measured in an in vitro assay. 1123. The method of embodiment 1121 or 1122, wherein the engineered guide RNA in the composition comprises a sequence selected from the group consisting of: SEQ ID NO: 1573, SEQ ID NO: 1588, SEQ ID NO: 1545, SEQ ID NO: 1575, SEQ ID NO: 1569, SEQ ID NO: 1584, SEQ ID NO: 1572, SEQ ID NO: 1567, SEQ ID NO: 1570, SEQ ID NO: 1587, SEQ ID NO: 1574, SEQ ID NO: 625, SEQ ID NO: 1571, SEQ ID NO: 874, SEQ ID NO: 17, SEQ ID NO: 1585, SEQ ID NO: 757, SEQ ID NO: 1581, SEQ ID NO: 1538, SEQ ID NO: 8, SEQ ID NO: 1002, SEQ ID NO: 1566, SEQ ID NO: 486, SEQ ID NO: 1552, SEQ ID NO: 505, SEQ ID NO: 635, SEQ ID NO: 606, SEQ ID NO: 884, SEQ ID NO: 1054, SEQ ID NO: 880, SEQ ID NO: 1411, SEQ ID NO: 1568, SEQ ID NO: 871, SEQ ID NO: 1580, SEQ ID NO: 1539, SEQ ID NO: 14, SEQ ID NO: 892, SEQ ID NO: 1116, SEQ ID NO: 15, SEQ ID NO: 1586, SEQ ID NO: 593, SEQ ID NO: 10, SEQ ID NO: 977, SEQ ID NO: 1578, SEQ ID NO: 1579, SEQ ID NO: 747, SEQ ID NO: 1577, 748, SEQ ID NO: 873, and SEQ ID NO: 494. 1124. The method of embodiment 1116, wherein the editing comprises editing at position 4 of the polyA tail of the DUX4 RNA. 1125. The method of embodiment 1124, wherein the editing comprises editing from about 54% to about 77% of the A at position 4 in the polyA tail of the DUX4 RNA as measured in an in vitro assay. 1126. The method of embodiment 1124 or 1125, wherein the engineered guide RNA in the composition comprises a sequence selected from the group consisting of: SEQ ID NO: 1575, SEQ ID NO: 1573, SEQ ID NO: 1567, SEQ ID NO: 1569, SEQ ID NO: 1570, SEQ ID NO: 1566, SEQ ID NO: 1572, SEQ ID NO: 1587, SEQ ID NO: 1571, SEQ ID NO: 1574, SEQ ID NO: 1584, SEQ ID NO: 1588, SEQ ID NO: 1054, SEQ ID NO: 1586, SEQ ID NO: 1585, SEQ ID NO: 1581, SEQ ID NO: 1578, SEQ ID NO: 1580, SEQ ID NO: 934, SEQ ID NO: 72, SEQ ID NO: 1582, SEQ ID NO: 1066, SEQ ID NO: 1183, SEQ ID NO: 1577, SEQ ID NO: 967, SEQ ID NO: 1568, SEQ ID NO: 930, SEQ ID NO: 566, SEQ ID NO: 1463, SEQ ID NO: 1294, SEQ ID NO: 1293, SEQ ID NO: 1391, SEQ ID NO: 1579, SEQ ID NO: 1583, SEQ ID NO: 944, SEQ ID NO: 815, SEQ ID NO: 1168, SEQ ID NO: 593, SEQ ID NO: 594, SEQ ID NO: 694, SEQ ID NO: 1576, SEQ ID NO: 1193, SEQ ID NO: 1051, SEQ ID NO: 1212, SEQ ID NO: 806, SEQ ID NO: 1059, SEQ ID NO: 1374, SEQ ID NO: 195, SEQ ID NO: 358, SEQ ID NO; and SEQ ID NO: 1296. 1127. The method of embodiment 1116, wherein the editing comprises editing at position 5 of the polyA tail of the DUX4 RNA. 1128. The method of embodiment 1127, wherein the editing comprises editing from about 44% to about 70% of the A at position 4 in the polyA tail of the DUX4 RNA as measured in an in vitro assay. 1129. The method embodiment 1127 or 1128, wherein the engineered guide RNA in the composition comprises a sequence selected from the group consisting of: SEQ ID NO: 1575, SEQ ID NO: 1573, SEQ ID NO: 1569, SEQ ID NO: 1574, SEQ ID NO: 1570, SEQ ID NO: 1572, SEQ ID NO: 1567, SEQ ID NO: 1587, SEQ ID NO: 1566, SEQ ID NO: 1571, SEQ ID NO: 1588, SEQ ID NO: 72, SEQ ID NO: 1586, SEQ ID NO: 1584, SEQ ID NO: 1581, SEQ ID NO: 1578, SEQ ID NO: 1585, SEQ ID NO: 1582, SEQ ID NO: 1580, SEQ ID NO: 1183, SEQ ID NO: 1568, SEQ ID NO: 1066, SEQ ID NO: 1391, SEQ ID NO: 1168, SEQ ID NO: 1293, SEQ ID NO: 1577, SEQ ID NO: 1054, SEQ ID NO: 566, SEQ ID NO: 1579, SEQ ID NO: 930, SEQ ID NO: 694, SEQ ID NO: 944, SEQ ID NO: 195, SEQ ID NO: 1583, SEQ ID NO: 815, SEQ ID NO: 1576, SEQ ID NO: 1051, SEQ ID NO: 1411, SEQ ID NO: 24, SEQ ID NO: 1163, SEQ ID NO: 935, SEQ ID NO: 680, SEQ ID NO: 1212, SEQ ID NO: 594, SEQ ID NO: 1185, SEQ ID NO: 1463, SEQ ID NO: 1058, SEQ ID NO: 810, SEQ ID NO: 392, SEQ ID NO; and SEQ ID NO: 1104. 1130. The method of embodiment 1115, wherein the DUX4 RNA comprises a pre-mRNA transcript of DUX4. 1131. The method of embodiment 1130, wherein at least 40%, at least 50%, at least 60%, at least 70%, at least 80%, at least 85%, at least 90%, at least 95%, or at least 99% of the pre-mRNA transcripts of DUX4 have at least one edit in the polyA signal sequence. 1132. The method of embodiment 1115, wherein the editing of DUX4 RNA facilitates a protein knockdown. 1133. The method of embodiment 1132, wherein the protein knockdown comprises a knockdown of DUX4. 1134. The method of embodiment 1132 or 1133, wherein the protein knockdown comprises a knockdown of a protein downstream of DUX4, wherein the protein downstream of DUX4 comprises SLC34A2, LEUTX, ZSCAN4, PRAMEF12, TRIM43, DEFB103, or MBD3L2, or any combination thereof. 1135. The composition of any one of embodiments 1132-1134, wherein the protein knockdown comprises a reduction of at least 40%, at least 50%, at least 60%, at least 70%, at least 80%, at least 85%, at least 90%, at least 95%, or at least 99% of the protein as compared to the protein level before RNA editing. 1136. The method of any one of embodiments 1132-1135, wherein an increased editing of the DUX4 RNA by the guide RNA is measured in an assay, wherein the increased editing comprises an increase in the protein knockdown. 1137. The composition of any one of embodiments 1-1105 or the pharmaceutical composition of embodiment 1106 for use as a medicament. 1138. The composition of any one of embodiments 1-1105 or the pharmaceutical composition of embodiment 1106 for use in the treatment of facioscapulohumeral muscular dystrophy (FSHD). 1139. The composition of embodiment 1138, wherein FSHD comprises Type I FSHD. 1140. The composition of embodiment 1138, wherein FSHD comprises Type II FSHD.

### EXAMPLES

The following illustrative examples are representative of embodiments of the stimulation, systems, and methods described herein and are not meant to be limiting in any way.

### Example 1

### Engineered Guide RNAs for Editing DUX4 TIS

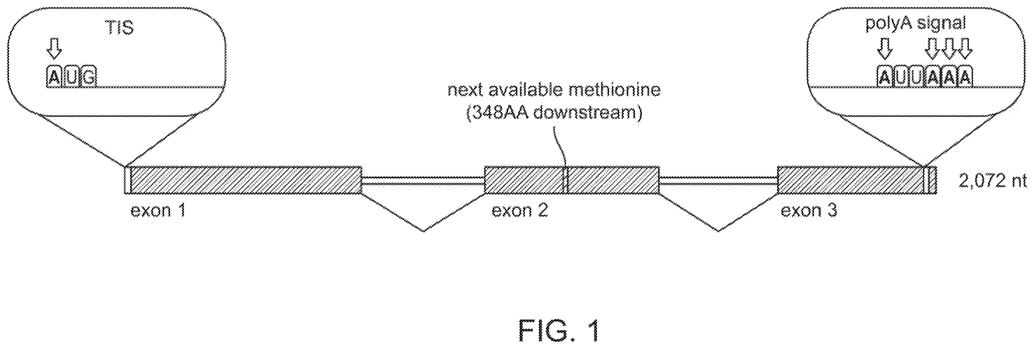

This example describes engineered guide RNAs for editing DUX4 RNA to knockdown expression of the DUX4 protein. A schematic of the DUX4 target is shown in FIG. 1, highlighting sites that can be targeted by engineered guide RNAs of the present disclosure. Engineered guide RNAs of the present disclosure are designed to target the single translation initiation site (TIS) of DUX4 RNA and facilitate ADAR-mediated RNA editing of AUG (the TIS) to GUG, thus, inhibiting DUX4 translation. Editing results in knockdown of the DUX4 protein, given that there are no other nearby methionine residues available for alternative translation initiation. Upon administration to of the engineered guide RNAs, in vitro or in vivo, the engineered guide RNAs edit the DUX4 TIS, thereby normalizing expression of DUX4 target genes. Upon administration to a subject having facioscapulohumeral muscular dystrophy (FSHD), the engineered guide RNAs are therapeutically effective and restore proper muscle function.

### Example 2

Engineered Guide RNAs for Editing DUX4 polyA Signal Site

This example describes engineered guide RNAs for editing DUX4 (DUX4-FL) RNA to knockdown expression of the corresponding DUX4 protein. A schematic of the DUX4 target is shown in FIG. 1, highlighting sites that can be targeted by engineered guide RNAs of the present disclosure. Engineered guide RNAs of the present disclosure are designed to target one or more adenosines in the single polyA signal sequence (ATTAAA) of DUX4-FL RNA and facilitate ADAR-mediated RNA editing of said one or more adenosines, thus, leading to disruption of RNA processing and inducement of degradation of the mRNA. This in turn leads to knockdown of the toxic DUX4-FL protein. Upon administration to of the engineered guide RNAs, in vitro or in vivo, the engineered guide RNAs edit the DUX4 polyA signal sequence, thereby normalizing expression of DUX4 target genes. Upon administration to a subject having facioscapulohumeral muscular dystrophy (FSHD), the engineered guide RNAs are therapeutically effective and restore proper muscle function.

### Example 3

Engineered Guide RNAs for Editing DMPK polyA Signal Site

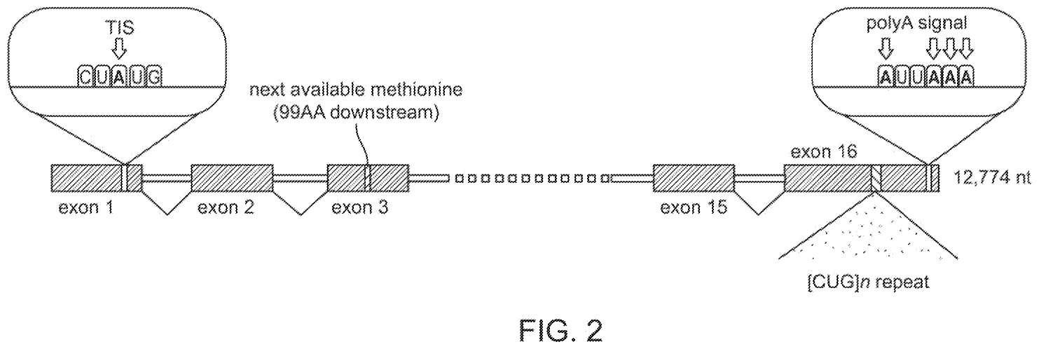

This example describes engineered guide RNAs for editing DMPK RNA to knockdown expression of myotonic dystrophy protein kinase. A schematic of the DMPK target is shown in FIG. 2, highlighting sites that can be targeted by engineered guide RNAs of the present disclosure. Engineered guide RNAs of the present disclosure are designed to target one or more adenosines in the polyA signal region of DMPK RNA and facilitate ADAR-mediated RNA editing of said one or more adenosines, thus, leading to disruption of RNA processing and inducement of degradation of the toxic mRNA. Upon administration to of the engineered guide RNAs, in vitro or in vivo, the engineered guide RNAs edit the DMPK polyA signal region. Upon administration to a subject having myotonic dystrophy (DM1), the engineered guide RNAs are therapeutically effective and prevent myotonia and muscle wasting.

### Example 4

### Engineered Guide RNAs for Editing DMPK TIS

This example describes engineered guide RNAs for editing DMPK RNA to knockdown expression of myotonic dystrophy protein kinase. A schematic of the DMPK target is shown in FIG. 2, highlighting sites that can be targeted by engineered guide RNAs of the present disclosure. Engineered guide RNAs of the present disclosure are designed to target the single translation initiation site (TIS) of DMPK RNA and facilitate ADAR-mediated RNA editing of AUG (the TIS) to GUG, thus, inhibiting DMPK translation. Editing results in knockdown of myotonic dystrophy protein kinase, given that there are no other nearby methionine residues available for alternative translation initiation. Upon administration to of the engineered guide RNAs, in vitro or in vivo, the engineered guide RNAs edit the DMPK TIS. Upon administration to a subject having myotonic dystrophy (DM1), the engineered guide RNAs are therapeutically effective and prevent myotonia and muscle wasting.

### Example 5

### Engineered Guide RNAs for Editing PMP22 TIS

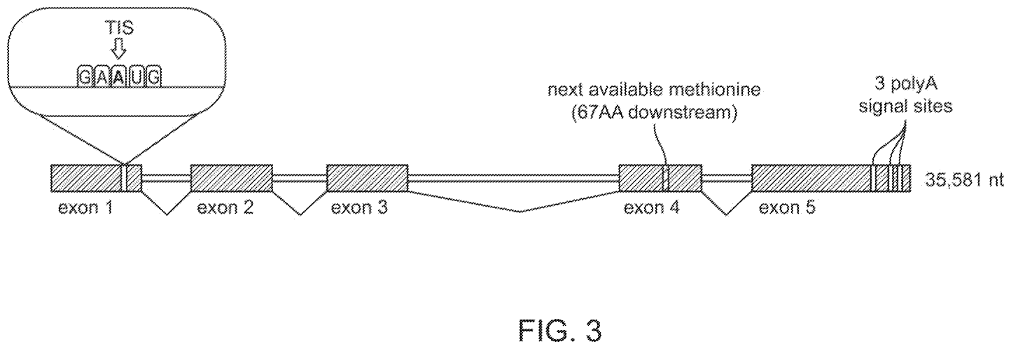

This example describes engineered guide RNAs for editing PMP22 RNA to knockdown expression of peripheral myelin protein-22 (PMP22). A schematic of the PMP22 target is shown in FIG. 3, highlighting sites that can be targeted by engineered guide RNAs of the present disclosure. Engineered guide RNAs of the present disclosure are designed to target the single translation initiation site (TIS) of PMP22 RNA and facilitate ADAR-mediated RNA editing of AUG (the TIS) to GUG, thus, inhibiting PMP22 translation. Editing results in knockdown of the PMP22 protein, given that there are no other nearby methionine residues available for alternative translation initiation. Upon administration to of the engineered guide RNAs, in vitro or in vivo, the engineered guide RNAs edit the PMP22 TIS. Upon administration to a subject having Charcot-Marie-Tooth Syndrome (CMT1A), the engineered guide RNAs are therapeutically effective and restore proper peripheral nerve myelination and conductance and improve muscle strength and sensory function.

### Example 6

Engineered Guide RNAs for Editing PMP22 polyA Signal Site

This example describes engineered guide RNAs for editing PMP22 RNA to knockdown expression of peripheral myelin protein-22 (PMP22). A schematic of the PMP22 target is shown in FIG. 3, highlighting sites that can be targeted by engineered guide RNAs of the present disclosure. One or more different engineered guide RNAs of the present disclosure are designed to target adenosines in one or more of the three alternative polyA signal sites of PMP22 RNA and facilitate ADAR-mediated RNA editing of said adenosines in said one or more of the three alternative polyA signal sites. Upon administration to of the engineered guide RNAs, in vitro or in vivo, the engineered guide RNAs edit one or more of the three alternative polyA signal sites. Upon administration to a subject having Charcot-Marie-Tooth Syndrome (CMT1A), the engineered guide RNAs are therapeutically effective and restore proper peripheral nerve myelination and conductance and improve muscle strength and sensory function.

### Example 7

### Engineered Guide RNAs for Editing SOD1 TIS

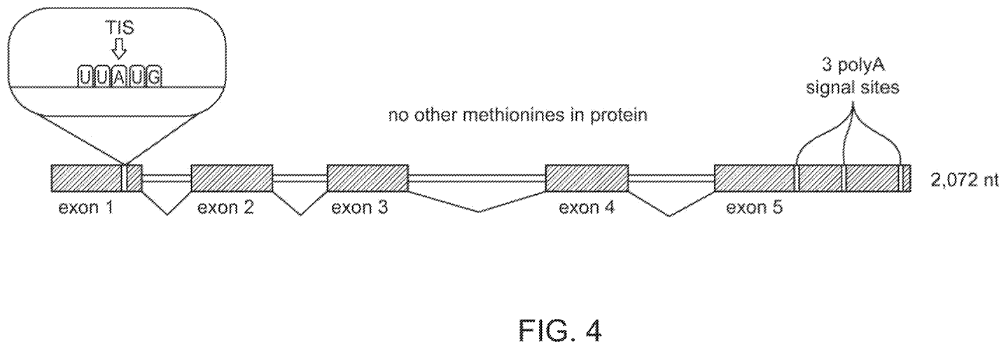

This example describes engineered guide RNAs for editing SOD1 RNA to knockdown expression of the superoxide dismutase enzyme. A schematic of the SOD1 target is shown in FIG. 4, highlighting sites that can be targeted by engineered guide RNAs of the present disclosure. Engineered guide RNAs of the present disclosure are designed to target the single translation initiation site (TIS) of SOD1 RNA and facilitate ADAR-mediated RNA editing of AUG (the TIS) to GUG, thus, inhibiting SOD1 translation and toxic protein function. Editing results in knockdown of the superoxide dismutase enzyme, given that there are no other nearby methionine residues available for alternative translation initiation. Upon administration to of the engineered guide RNAs, in vitro or in vivo, the engineered guide RNAs edit the SOD1 TIS. Upon administration to a subject having amyotrophic lateral sclerosis (ALS), the engineered guide RNAs are therapeutically effective and prevent motor neuron degeneration and disease progression.

### Example 8

Engineered Guide RNAs for Editing SOD1 polyA Signal Site

This example describes engineered guide RNAs for editing SOD1 RNA to knockdown expression of the superoxide dismutase enzyme. A schematic of the SOD1 target is shown in FIG. 4, highlighting sites that can be targeted by engineered guide RNAs of the present disclosure. One or more different engineered guide RNAs of the present disclosure are designed to target adenosines in one or more of the three alternative polyA signal sites of SOD1 RNA and facilitate ADAR-mediated RNA editing of said adenosines in said one or more of the three alternative polyA signal sites. Upon administration to of the engineered guide RNAs, in vitro or in vivo, the engineered guide RNAs edit one or more of the three alternative polyA signal sites. Upon administration to a subject having amyotrophic lateral sclerosis (ALS), the engineered guide RNAs are therapeutically effective and prevent motor neuron degeneration and disease progression.

### Example 9

### Engineered Guide RNA Compositions Targeting DUX4

This example describes engineered guide RNAs that target the polyadenylation (polyA) signal site (ATTAAA) in the “pLAM” region of DUX4 mRNA. One or more of the three terminal As in the polyA signal site sequence (ATTAAA) was targeted for editing using the engineered guide RNA sequences of TABLE 1. The results from the DUX4 polyA signal site editing (percent editing of an indicated A) are shown in TABLE 2. TABLE 2 shows the percent editing of As in ATTAAA of DUX4 mRNA by ADAR1 (A1), ADAR2 (A2), or ADAR1 and ADAR2 (A1+2) with the guide RNAs described in TABLE 1. Position 0 (the first A of ATTAAA) is indicated as “P0”, position 3 (the third A of ATTAAA) is indicated as “P3”, position 4 (the fourth A of ATTAAA) is indicated as “P4”, position 5 (the fifth A of ATTAAA) is indicated as “P5”, and editing at any of the locations is indicated as “any” in TABLE 2. Self-annealing RNA structures, which comprised (i) the engineered guide RNAs shown in TABLE 1 and (ii) the RNA sequences of the DUX4 region targeted by the engineered guide RNAs, were contacted with an RNA editing entity (e.g., a recombinant ADAR1 and/or ADAR2) for 30 minutes under conditions that allowed for editing. The regions targeted by the engineered guide RNAs were subsequently assessed for editing using next generation sequencing (NGS). Engineered guide RNAs that displayed favorable on-target editing of DUX4 for ADAR1 and/or ADAR2 are shown in TABLE 1. All polynucleotide sequences encoding for the engineered guide RNAs of TABLE 1, are also encompassed herein, which are represented by each of the sequences shown in TABLE 1, with a T substituted for each U. For each sequence, the structural features formed in the double stranded RNA substrate upon hybridization of the guide RNA to the target DUX4 RNA, are shown in the second column of TABLE 1. For reference, each structural feature formed within a guide-target RNA scaffold (target RNA sequence hybridized to an engineered guide RNA) is annotated as follows:
a. the position of the structural feature with respect to the target A (position 0) of the target RNA sequence, with a negative value indicating upstream (5′) of the target A and a positive value indicating downstream (3′) of the target A;
b. the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c. the name of the structural feature (e.g., symmetric bulge, symmetric internal loop, asymmetric bulge, asymmetric internal loop, mismatch, or wobble base pair), and
d. the sequences of bases on the target RNA side and the engineered guide RNA side that participate in forming the structural feature.

For example, with reference to SEQ ID NO: 7, “20_6-6_internal_loop-symmetric_UGGAUC-UACAUU” is read as a structural feature formed in a guide-target RNA scaffold (target DUX4 RNA sequence hybridized to an engineered guide RNA of SEQ ID NO: 7), where
a. the structural feature starts 20 nucleotides downstream (3′) (the +20 position) from the target A (0 position) of the target RNA sequence
b. six contiguous bases from the target RNA sequence and six contiguous bases from the engineered guide RNA form the structural feature
c. the structural feature is an internal symmetric loop
d. a sequence of UGGAUC from the target RNA side and a sequence of UACAUU from the engineered guide RNA side participate in forming the internal symmetric loop.

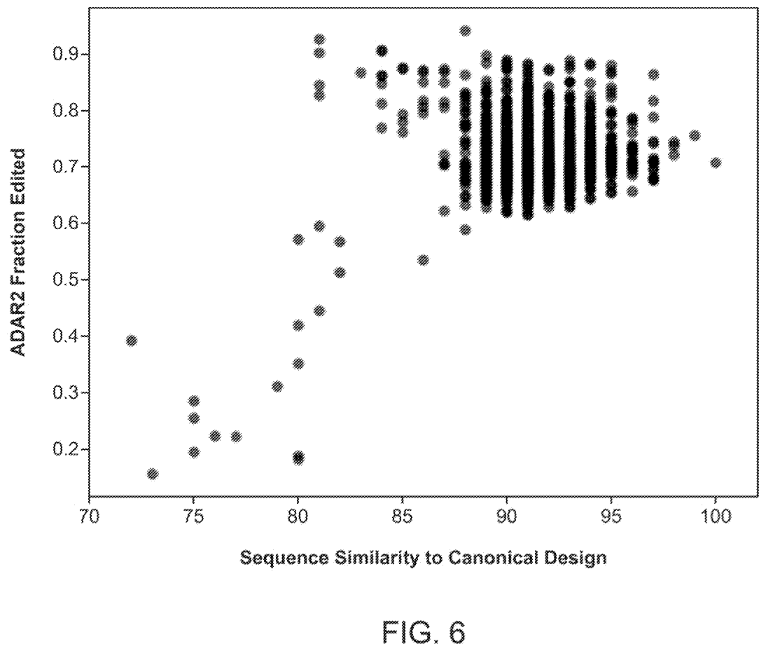

For reference, FIG. 5 can be used as an aid to visualize the structural features and the nomenclature disclosed herein. FIG. 6 is a plot showing, on the x-axis, the sequence similarity of the DUX4-targeting engineered guide RNA sequences of the present disclosure to a canonical guide RNA design and, on the y-axis, the edited fraction by an ADAR2 enzyme. These data highlight the diverse sequence space represented by the DUX4-targeting engineered guide RNA sequences of the present disclosure, which have a range of different structural features that drive sequence diversity, and which exhibit high on-target editing efficiency.

TABLE 1
Engineered Guide RNAs Targeting DUX4

| SEQ | | |
|---|---|---|
| ID | Structural | Engineered Guide |
| NO | Features | RNA sequence |
| 2 | 0_1-1_mismatch_ | GAUAUUGUGACAUAUCUCUGCACUCAU |
| | A-C | CACACAAAAGAUGCAAAUCUUCUAUAG |
| | | GAUCCACAGGGAGGGGGCAUUUUAAC |
| | | AUAUCUCUGAACUAAUCAUC |
| 3 | 3_1-1_mismatch_ | GAUAUUGUGACAUAUCUCUGCACUCAU |
| | A-C | CACACAAAAGAUGCAAAUCUUCUAUAG |
| | | GAUCCACAGGGAGGGGGCAUUUCAAU |
| | | AUAUCUCUGAACUAAUCAUC |
| 4 | 4_1-1_mismatch_ | GAUAUUGUGACAUAUCUCUGCACUCAU |
| | A-C | CACACAAAAGAUGCAAAUCUUCUAUAG |
| | | GAUCCACAGGGAGGGGGCAUUCUAAU |
| | | AUAUCUCUGAACUAAUCAUC |
| 5 | 5_1-1_mismatch_ | GAUAUUGUGACAUAUCUCUGCACUCAU |
| | A-C | CACACAAAAGAUGCAAAUCUUCUAUAG |
| | | GAUCCACAGGGAGGGGGCAUCUUAAU |
| | | AUAUCUCUGAACUAAUCAUC |
| 6 | | GAUAUUGUGACAUAUCUCUGCACUCAU |
| | | CACACAAAAGAUGCAAAUCUUCUAUAG |
| | | GAUCCACAGGGAGGGGGCAUUUUAAU |
| | | AUAUCUCUGAACUAAUCAUC |
| 7 | 0_1-1_mismatch_ | GAUAUUGUGACAUAUCUCUGCACUCAU |
| | A-C | CACACAAAAGAUGCAAAUCUUCUAUAG |
| | 20_6-6_internal_ | UACAUUCAGGGAGGGGGCAUUUUAAC |
| | loop-symmetric_ | AUAUCUCUGAACUAAUCAUC |
| | UGGAUC-UACAUU | |
| 8 | 3_1-1_mismatch_ | GAUAUUGUGACAUAUCUCUGCACUCAU |
| | A-C | CACACAAAAGAUGCAAAUCUUCUAUAG |
| | 20_6-6_internal_ | ACAGGUCAGGGAGGGGGCAUUUCAAU |
| | loop-symmetric_ | AUAUCUCUGAACUAAUCAUC |
| | UGGAUC-ACAGGU | |
| 9 | 4_1-1_mismatch_ | GAUAUUGUGACAUAUCUCUGCACUCAU |
| | A-C | CACACAAAAGAUGCAAAUCUUCUAUAG |
| | 20_6-6_internal_ | CACAAUCAGGGAGGGGGCAUUCUAAU |
| | loop-symmetric_ | AUAUCUCUGAACUAAUCAUC |
| | UGGAUC-CACAAU | |
| 10 | 5_1-1_mismatch_ | GAUAUUGUGACAUAUCUCUGCACUCAU |
| | A-C | CACACAAAAGAUGCAAAUCUUCUAUAG |
| | 20_6-6_internal_ | UUAGAUCAGGGAGGGGGCAUCUUAAU |
| | loop-symmetric_ | AUAUCUCUGAACUAAUCAUC |
| | UGGAUC-UUAGAU | |
| 11 | 20_6-6_internal_ | GAUAUUGUGACAUAUCUCUGCACUCAU |
| | loop-symmetric_ | CACACAAAAGAUGCAAAUCUUCUAUAG |
| | UGGAUC-AAAAUU | AAAAUUCAGGGAGGGGGCAUUUUAAU |
| | | AUAUCUCUGAACUAAUCAUC |
| 12 | 0_1-1_mismatch_ | GAUAUUGUGACAUAUCUCUGCACUCAU |
| | A-C | CACACAAAAGAUGCAAAUCUUCUAUAA |
| | 21_6-6_internal_ | ACAUAACAGGGAGGGGGCAUUUUAAC |
| | loop-symmetric_ | AUAUCUCUGAACUAAUCAUC |
| | GGAUCC-AACAUA | |
| 13 | 3_1-1_mismatch_ | GAUAUUGUGACAUAUCUCUGCACUCAU |
| | A-C | CACACAAAAGAUGCAAAUCUUCUAUAU |
| | 21_6-6_internal_ | AAGUAACAGGGAGGGGGCAUUUCAAU |
| | loop-symmetric_ | AUAUCUCUGAACUAAUCAUC |
| | GGAUCC-UAAGUA | |
| 14 | 5_1-1_mismatch_ | GAUAUUGUGACAUAUCUCUGCACUCAU |
| | A-C | CACACAAAAGAUGCAAAUCUUCUAUAU |
| | 21_6-6_internal_ | CGCGGACAGGGAGGGGGCAUCUUAAU |
| | loop-symmetric_ | AUAUCUCUGAACUAAUCAUC |
| | GGAUCC-UCGCGG | |
| 15 | 21_6-6_internal_ | GAUAUUGUGACAUAUCUCUGCACUCAU |
| | loop-symmetric_ | CACACAAAAGAUGCAAAUCUUCUAUAC |
| | GGAUCC-CCCCAA | CCCAAACAGGGAGGGGGCAUUUUAAU |
| | | AUAUCUCUGAACUAAUCAUC |
| 16 | 0_1-1_mismatch_ | GAUAUUGUGACAUAUCUCUGCACUCAU |
| | A-C | CACACAAAAGAUGCAAAUCUUCUAUUG |
| | 22_6-6_internal_ | UCGACACAGGGAGGGGGCAUUUUAAC |
| | loop-symmetric_ | AUAUCUCUGAACUAAUCAUC |
| | GAUCCU-UGUCGA | |
| 17 | 3_1-1_mismatch_ | GAUAUUGUGACAUAUCUCUGCACUCAU |
| | A-C | CACACAAAAGAUGCAAAUCUUCUAUCC |
| | 22_6-6_internal_ | CCGACACAGGGAGGGGGCAUUUCAAUA |
| | loop-symmetric_ | UAUCUCUGAACUAAUCAUC |
| | GAUCCU-CCCCGA | |
| 18 | 4_1-1_mismatch_ | GAUAUUGUGACAUAUCUCUGCACUCAU |
| | A-C | CACACAAAAGAUGCAAAUCUUCUAUUU |
| | 22_6-6_internal_ | CUAACACAGGGAGGGGGCAUUCUAAU |
| | loop-symmetric_ | AUAUCUCUGAACUAAUCAUC |
| | GAUCCU-UUCUAA | |
| 19 | 5_1-1_mismatch_ | GAUAUUGUGACAUAUCUCUGCACUCAU |
| | A-C | CACACAAAAGAUGCAAAUCUUCUAUCA |
| | 22_6-6_internal_ | CCGACACAGGGAGGGGGCAUCUUAAUA |
| | loop-symmetric_ | UAUCUCUGAACUAAUCAUC |
| | GAUCCU-CACCGA | |
| 20 | 22_6-6_internal_ | GAUAUUGUGACAUAUCUCUGCACUCAU |
| | loop-symmetric_ | CACACAAAAGAUGCAAAUCUUCUAUUA |
| | GAUCCU-UACUAA | CUAACACAGGGAGGGGGCAUUUUAAU |
| | | AUAUCUCUGAACUAAUCAUC |
| 21 | 0_1-1_mismatch_ | GAUAUUGUGACAUAUCUCUGCACUCAU |
| | A-C | CACACAAAAGAUGCAAAUCUUCUAGAU |
| | 23_6-6_internal_ | CUCCCACAGGGAGGGGGCAUUUUAACA |
| | loop-symmetric_ | UAUCUCUGAACUAAUCAUC |
| | AUCCUA-GAUCUC | |
| 22 | 3_1-1_mismatch_ | GAUAUUGUGACAUAUCUCUGCACUCAU |
| | A-C | CACACAAAAGAUGCAAAUCUUCUAGUC |
| | 23_6-6_internal_ | ACGCCACAGGGAGGGGGCAUUUCAAUA |
| | loop-symmetric_ | UAUCUCUGAACUAAUCAUC |
| | AUCCUA-GUCACG | |
| 23 | 4_1-1_mismatch_ | GAUAUUGUGACAUAUCUCUGCACUCAU |
| | A-C | CACACAAAAGAUGCAAAUCUUCUAAUA |
| | 23_6-6_internal_ | UCCCCACAGGGAGGGGGCAUUCUAAUA |
| | loop-symmetric_ | UAUCUCUGAACUAAUCAUC |
| | AUCCUA-AUAUCC | |
| 24 | 5_1-1_mismatch_ | GAUAUUGUGACAUAUCUCUGCACUCAU |
| | A-C | CACACAAAAGAUGCAAAUCUUCUACGU |
| | 23_6-6_internal_ | CCGCCACAGGGAGGGGGCAUCUUAAUA |
| | loop-symmetric_ | UAUCUCUGAACUAAUCAUC |
| | AUCCUA-CGUCCG | |
| 25 | 23_6-6_internal_ | GAUAUUGUGACAUAUCUCUGCACUCAU |
| | loop-symmetric_ | CACACAAAAGAUGCAAAUCUUCUAGAA |
| | AUCCUA-GAAUCG | UCGCCACAGGGAGGGGGCAUUUUAAU |
| | | AUAUCUCUGAACUAAUCAUC |
| 26 | 0_1-1_mismatch_ | GAUAUUGUGACAUAUCUCUGCACUCAU |
| | A-C | CACACAAAAGAUGCAAAUCUUCUUACA |
| | 24_6-6_internal_ | GUUCCACAGGGAGGGGGCAUUUUAAC |
| | loop-symmetric_ | AUAUCUCUGAACUAAUCAUC |
| | UCCUAU-UACAGU | |
| 27 | 3_1-1_mismatch_ | GAUAUUGUGACAUAUCUCUGCACUCAU |
| | A-C | CACACAAAAGAUGCAAAUCUUCUUCUA |
| | 24_6-6_internal_ | CUUCCACAGGGAGGGGGCAUUUCAAUA |
| | loop-symmetric_ | UAUCUCUGAACUAAUCAUC |
| | UCCUAU-UCUACU | |
| 28 | 4_1-1_mismatch_ | GAUAUUGUGACAUAUCUCUGCACUCAU |
| | A-C | CACACAAAAGAUGCAAAUCUUCUUCGC |
| | 24_6-6_internal_ | GUUCCACAGGGAGGGGGCAUUCUAAU |
| | loop-symmetric_ | AUAUCUCUGAACUAAUCAUC |
| | UCCUAU-UCGCGU | |
| 29 | 5_1-1_mismatch_ | GAUAUUGUGACAUAUCUCUGCACUCAU |
| | A-C | CACACAAAAGAUGCAAAUCUUCUCCGU |
| | 24_6-6_internal_ | AUUCCACAGGGAGGGGGCAUCUUAAU |
| | loop-symmetric_ | AUAUCUCUGAACUAAUCAUC |
| | UCCUAU-CCGUAU | |
| 30 | 24_6-6_internal_ | GAUAUUGUGACAUAUCUCUGCACUCAU |
| | loop-symmetric_ | CACACAAAAGAUGCAAAUCUUCUUGUC |
| | UCCUAU-UGUCUC | UCUCCACAGGGAGGGGGCAUUUUAAU |
| | | AUAUCUCUGAACUAAUCAUC |
| 31 | 0_1-1_mismatch_ | GAUAUUGUGACAUAUCUCUGCACUCAU |
| | A-C | CACACAAAAGAUGCAAAUCUUCACCGA |
| | 25_6-6_internal_ | CAUCCACAGGGAGGGGGCAUUUUAACA |
| | loop-symmetric_ | UAUCUCUGAACUAAUCAUC |
| | CCUAUA-ACCGAC | |
| 32 | 3_1-1_mismatch_ | GAUAUUGUGACAUAUCUCUGCACUCAU |
| | A-C | CACACAAAAGAUGCAAAUCUUCCCAUU |
| | 25_6-6_internal_ | UAUCCACAGGGAGGGGGCAUUUCAAU |
| | loop-symmetric_ | AUAUCUCUGAACUAAUCAUC |
| | CCUAUA-CCAUUU | |
| 33 | 4_1-1_mismatch_ | GAUAUUGUGACAUAUCUCUGCACUCAU |
| | A-C | CACACAAAAGAUGCAAAUCUUCCCUUU |
| | 25_6-6_internal_ | AAUCCACAGGGAGGGGGCAUUCUAAU |
| | loop-symmetric_ | AUAUCUCUGAACUAAUCAUC |
| | CCUAUA-CCUUUA | |
| 34 | 5_1-1_mismatch_ | GAUAUUGUGACAUAUCUCUGCACUCAU |
| | A-C | CACACAAAAGAUGCAAAUCUUCAUCGU |
| | 25_6-6_internal_ | UAUCCACAGGGAGGGGGCAUCUUAAU |
| | loop-symmetric_ | AUAUCUCUGAACUAAUCAUC |
| | CCUAUA-AUCGUU | |
| 35 | 25_6-6_internal_ | GAUAUUGUGACAUAUCUCUGCACUCAU |
| | loop-symmetric_ | CACACAAAAGAUGCAAAUCUUCAAGAA |
| | CCUAUA-AAGAAA | AAUCCACAGGGAGGGGGCAUUUUAAU |
| | | AUAUCUCUGAACUAAUCAUC |
| 36 | 0_1-1_mismatch_ | GAUAUUGUGACAUAUCUCUGCACUCAU |
| | A-C | CACACAAAAGAUGCAAAUCUUAAUGAC |
| | 26_6-6_internal_ | GAUCCACAGGGAGGGGGCAUUUUAAC |
| | loop-symmetric_ | AUAUCUCUGAACUAAUCAUC |
| | CUAUAG-AAUGAC | |
| 37 | 3_1-1_mismatch_ | GAUAUUGUGACAUAUCUCUGCACUCAU |
| | A-C | CACACAAAAGAUGCAAAUCUUGGGCCA |
| | 26_6-6_internal_ | GAUCCACAGGGAGGGGGCAUUUCAAU |
| | loop-symmetric_ | AUAUCUCUGAACUAAUCAUC |
| | CUAUAG-GGGCCA | |
| 38 | 4_1-1_mismatch_ | GAUAUUGUGACAUAUCUCUGCACUCAU |
| | A-C | CACACAAAAGAUGCAAAUCUUAGUGUC |
| | 26_6-6_internal_ | GAUCCACAGGGAGGGGGCAUUCUAAU |
| | loop-symmetric_ | AUAUCUCUGAACUAAUCAUC |
| | CUAUAG-AGUGUC | |
| 39 | 5_1-1_mismatch_ | GAUAUUGUGACAUAUCUCUGCACUCAU |
| | A-C | CACACAAAAGAUGCAAAUCUUAGCUUU |
| | 26_6-6_internal_ | GAUCCACAGGGAGGGGGCAUCUUAAU |
| | loop-symmetric_ | AUAUCUCUGAACUAAUCAUC |
| | CUAUAG-AGCUUU | |
| 40 | 26_6-6_internal_ | GAUAUUGUGACAUAUCUCUGCACUCAU |
| | loop-symmetric_ | CACACAAAAGAUGCAAAUCUUGGAAU |
| | CUAUAG-GGAAUU | UGAUCCACAGGGAGGGGGCAUUUUAA |
| | | UAUAUCUCUGAACUAAUCAUC |
| 41 | 0_1-1_mismatch_ | GAUAUUGUGACAUAUCUCUGCACUCAU |
| | A-C | CACACAAAAGAUGCAAAUCUCAGUCUG |
| | 27_6-6_internal_ | GAUCCACAGGGAGGGGGCAUUUUAAC |
| | loop-symmetric_ | AUAUCUCUGAACUAAUCAUC |
| | UAUAGA-CAGUCU | |
| 42 | 3_1-1_mismatch_ | GAUAUUGUGACAUAUCUCUGCACUCAU |
| | A-C | CACACAAAAGAUGCAAAUCUAACCAUG |
| | 27_6-6_internal_ | GAUCCACAGGGAGGGGGCAUUUCAAU |
| | loop-symmetric_ | AUAUCUCUGAACUAAUCAUC |
| | UAUAGA-AACCAU | |
| 43 | 4_1-1_mismatch_ | GAUAUUGUGACAUAUCUCUGCACUCAU |
| | A-C | CACACAAAAGAUGCAAAUCUCCACCUG |
| | 27_6-6_internal_ | GAUCCACAGGGAGGGGGCAUUCUAAU |
| | loop-symmetric_ | AUAUCUCUGAACUAAUCAUC |
| | UAUAGA-CCACCU | |
| 44 | 5_1-1_mismatch_ | GAUAUUGUGACAUAUCUCUGCACUCAU |
| | A-C | CACACAAAAGAUGCAAAUCUGUGCACG |
| | 27_6-6_internal_ | GAUCCACAGGGAGGGGGCAUCUUAAU |
| | loop-symmetric_ | AUAUCUCUGAACUAAUCAUC |
| | UAUAGA-GUGCAC | |
| 45 | 27_6-6_internal_ | GAUAUUGUGACAUAUCUCUGCACUCAU |
| | loop-symmetric_ | CACACAAAAGAUGCAAAUCUGACAUCG |
| | UAUAGA-GACAUC | GAUCCACAGGGAGGGGGCAUUUUAAU |
| | | AUAUCUCUGAACUAAUCAUC |
| 46 | 0_1-1_mismatch_ | GAUAUUGUGACAUAUCUCUGCACUCAU |
| | A-C | CACACAAAAGAUGCAAAUCCAGUCAAG |
| | 28_6-6_internal_ | GAUCCACAGGGAGGGGGCAUUUUAAC |
| | loop-symmetric_ | AUAUCUCUGAACUAAUCAUC |
| | AUAGAA-CAGUCA | |
| 47 | 3_1-1_mismatch_ | GAUAUUGUGACAUAUCUCUGCACUCAU |
| | A-C | CACACAAAAGAUGCAAAUCACUCCGAG |
| | 28_6-6_internal_ | GAUCCACAGGGAGGGGGCAUUUCAAU |
| | loop-symmetric_ | AUAUCUCUGAACUAAUCAUC |
| | AUAGAA-ACUCCG | |
| 48 | 4_1-1_mismatch_ | GAUAUUGUGACAUAUCUCUGCACUCAU |
| | A-C | CACACAAAAGAUGCAAAUCCGAAACAG |
| | 28_6-6_internal_ | GAUCCACAGGGAGGGGGCAUUCUAAU |
| | loop-symmetric_ | AUAUCUCUGAACUAAUCAUC |
| | AUAGAA-CGAAAC | |
| 49 | 5_1-1_mismatch_ | GAUAUUGUGACAUAUCUCUGCACUCAU |
| | A-C | CACACAAAAGAUGCAAAUCAUACGAAG |
| | 28_6-6_internal_ | GAUCCACAGGGAGGGGGCAUCUUAAU |
| | loop-symmetric_ | AUAUCUCUGAACUAAUCAUC |
| | AUAGAA-AUACGA | |
| 50 | 28_6-6_internal_ | GAUAUUGUGACAUAUCUCUGCACUCAU |
| | loop-symmetric_ | CACACAAAAGAUGCAAAUCCCGGGGAG |
| | AUAGAA-CCGGGG | GAUCCACAGGGAGGGGGCAUUUUAAU |
| | | AUAUCUCUGAACUAAUCAUC |
| 51 | 0_1-1_mismatch_ | GAUAUUGUGACAUAUCUCUGCACUCAU |
| | A-C | CACACAAAAGAUGCAAAUAUUGACUA |
| | 29_6-6_internal_ | GGAUCCACAGGGAGGGGGCAUUUUAA |
| | loop-symmetric_ | CAUAUCUCUGAACUAAUCAUC |
| | UAGAAG-AUUGAC | |
| 52 | 3_1-1_mismatch_ | GAUAUUGUGACAUAUCUCUGCACUCAU |
| | A-C | CACACAAAAGAUGCAAAUGACCCCUAG |
| | 29_6-6_internal_ | GAUCCACAGGGAGGGGGCAUUUCAAU |
| | loop-symmetric_ | AUAUCUCUGAACUAAUCAUC |
| | UAGAAG-GACCCC | |
| 53 | 4_1-1_mismatch_ | GAUAUUGUGACAUAUCUCUGCACUCAU |
| | A-C | CACACAAAAGAUGCAAAUGCGUGCUAG |
| | 29_6-6_internal_ | GAUCCACAGGGAGGGGGCAUUCUAAU |
| | loop-symmetric_ | AUAUCUCUGAACUAAUCAUC |
| | UAGAAG-GCGUGC | |
| 54 | 5_1-1_mismatch_ | GAUAUUGUGACAUAUCUCUGCACUCAU |
| | A-C | CACACAAAAGAUGCAAAUGAGGACUA |
| | 29_6-6_internal_ | GGAUCCACAGGGAGGGGGCAUCUUAA |
| | loop-symmetric_ | UAUAUCUCUGAACUAAUCAUC |
| | UAGAAG-GAGGAC | |
| 55 | 29_6-6_internal_ | GAUAUUGUGACAUAUCUCUGCACUCAU |
| | loop-symmetric_ | CACACAAAAGAUGCAAAUGGGAAUUA |
| | UAGAAG-GGGAAU | GGAUCCACAGGGAGGGGGCAUUUUAA |
| | | UAUAUCUCUGAACUAAUCAUC |
| 56 | 0_1-1_mismatch_ | GAUAUUGUGACAUAUCUCUGCACUCAU |
| | A-C | CACACAAAAGAUGCAAAAGAGAGAUA |
| | 30_6-6_internal_ | GGAUCCACAGGGAGGGGGCAUUUUAA |
| | loop-symmetric_ | CAUAUCUCUGAACUAAUCAUC |
| | AGAAGA-AGAGAG | |
| 57 | 3_1-1_mismatch_ | GAUAUUGUGACAUAUCUCUGCACUCAU |
| | A-C | CACACAAAAGAUGCAAACGCACGAUAG |
| | 30_6-6_internal_ | GAUCCACAGGGAGGGGGCAUUUCAAU |
| | loop-symmetric_ | AUAUCUCUGAACUAAUCAUC |
| | AGAAGA-CGCACG | |
| 58 | 4_1-1_mismatch_ | GAUAUUGUGACAUAUCUCUGCACUCAU |
| | A-C | CACACAAAAGAUGCAAACCAGAGAUAG |
| | 30_6-6_internal_ | GAUCCACAGGGAGGGGGCAUUCUAAU |
| | loop-symmetric_ | AUAUCUCUGAACUAAUCAUC |
| | AGAAGA-CCAGAG | |
| 59 | 5_1-1_mismatch_ | GAUAUUGUGACAUAUCUCUGCACUCAU |
| | A-C | CACACAAAAGAUGCAAAAGGCGAAUA |
| | 30_6-6_internal_ | GGAUCCACAGGGAGGGGGCAUCUUAA |
| | loop-symmetric_ | UAUAUCUCUGAACUAAUCAUC |
| | AGAAGA-AGGCGA | |
| 60 | 30_6-6_internal_ | GAUAUUGUGACAUAUCUCUGCACUCAU |
| | loop-symmetric_ | CACACAAAAGAUGCAAAGCAAGAAUA |
| | AGAAGA-GCAAGA | GGAUCCACAGGGAGGGGGCAUUUUAA |
| | | UAUAUCUCUGAACUAAUCAUC |
| 61 | 0_1-1_mismatch_ | GAUAUUGUGACAUAUCUCUGCACUCAU |
| | A-C | CACACAAAAGAUGCAACCCCGAUAUAG |
| | 31_6-6_internal_ | GAUCCACAGGGAGGGGGCAUUUUAAC |
| | loop-symmetric_ | AUAUCUCUGAACUAAUCAUC |
| | GAAGAU-CCCCGA | |
| 62 | 3_1-1_mismatch_ | GAUAUUGUGACAUAUCUCUGCACUCAU |
| | A-C | CACACAAAAGAUGCAACUAAAAUAUA |
| | 31_6-6_internal_ | GGAUCCACAGGGAGGGGGCAUUUCAA |
| | loop-symmetric_ | UAUAUCUCUGAACUAAUCAUC |
| | GAAGAU-CUAAAA | |
| 63 | 4_1-1_mismatch_ | GAUAUUGUGACAUAUCUCUGCACUCAU |
| | A-C | CACACAAAAGAUGCAACGUGGGUAUA |
| | 31_6-6_internal_ | GGAUCCACAGGGAGGGGGCAUUCUAA |
| | loop-symmetric_ | UAUAUCUCUGAACUAAUCAUC |
| | GAAGAU-CGUGGG | |
| 64 | 5_1-1_mismatch_ | GAUAUUGUGACAUAUCUCUGCACUCAU |
| | A-C | CACACAAAAGAUGCAACUGGGAUAUA |
| | 31_6-6_internal_ | GGAUCCACAGGGAGGGGGCAUCUUAA |
| | loop-symmetric_ | UAUAUCUCUGAACUAAUCAUC |
| | GAAGAU-CUGGGA | |
| 65 | 31_6-6_internal_ | GAUAUUGUGACAUAUCUCUGCACUCAU |
| | loop-symmetric_ | CACACAAAAGAUGCAAUGGAGAUAUA |
| | GAAGAU-UGGAGA | GGAUCCACAGGGAGGGGGCAUUUUAA |
| | | UAUAUCUCUGAACUAAUCAUC |
| 66 | 0_1-1_mismatch_ | GAUAUUGUGACAUAUCUCUGCACUCAU |
| | A-C | CACACAAAAGAUGCAUCCGGACUAUAG |
| | 32_6-6_internal_ | GAUCCACAGGGAGGGGGCAUUUUAAC |
| | loop-symmetric_ | AUAUCUCUGAACUAAUCAUC |
| | AAGAUU-UCCGGA | |
| 67 | 3_1-1_mismatch_ | GAUAUUGUGACAUAUCUCUGCACUCAU |
| | A-C | CACACAAAAGAUGCAUCCUGGCUAUAG |
| | 32_6-6_internal_ | GAUCCACAGGGAGGGGGCAUUUCAAU |
| | loop-symmetric_ | AUAUCUCUGAACUAAUCAUC |
| | AAGAUU-UCCUGG | |
| 68 | 4_1-1_mismatch_ | GAUAUUGUGACAUAUCUCUGCACUCAU |
| | A-C | CACACAAAAGAUGCACCUAGACUAUAG |
| | 32_6-6_internal_ | GAUCCACAGGGAGGGGGCAUUCUAAU |
| | loop-symmetric_ | AUAUCUCUGAACUAAUCAUC |
| | AAGAUU-CCUAGA | |
| 69 | 5_1-1_mismatch_ | GAUAUUGUGACAUAUCUCUGCACUCAU |
| | A-C | CACACAAAAGAUGCACCCGGACUAUAG |
| | 32_6-6_internal_ | GAUCCACAGGGAGGGGGCAUCUUAAU |
| | loop-symmetric_ | AUAUCUCUGAACUAAUCAUC |
| | AAGAUU-CCCGGA | |
| 70 | 32_6-6_internal_ | GAUAUUGUGACAUAUCUCUGCACUCAU |
| | loop-symmetric_ | CACACAAAAGAUGCACUGAGGCUAUAG |
| | AAGAUU-CUGAGG | GAUCCACAGGGAGGGGGCAUUUUAAU |
| | | AUAUCUCUGAACUAAUCAUC |
| 71 | 0_1-1_mismatch_ | GAUAUUGUGACAUAUCUCUGCACUCAU |
| | A-C | CACACAAAAGAUGCUUGAGAUCUAUA |
| | 33_6-6_internal_ | GGAUCCACAGGGAGGGGGCAUUUUAA |
| | loop-symmetric_ | CAUAUCUCUGAACUAAUCAUC |
| | AGAUUU-UUGAGA | |
| 72 | 3_1-1_mismatch_ | GAUAUUGUGACAUAUCUCUGCACUCAU |
| | A-C | CACACAAAAGAUGCUUGGGAUCUAUA |
| | 33_6-6_internal_ | GGAUCCACAGGGAGGGGGCAUUUCAA |
| | loop-symmetric_ | UAUAUCUCUGAACUAAUCAUC |
| | AGAUUU-UUGGGA | |
| 73 | 4_1-1_mismatch_ | GAUAUUGUGACAUAUCUCUGCACUCAU |
| | A-C | CACACAAAAGAUGCUUUGCGUCUAUAG |
| | 33_6-6_internal_ | GAUCCACAGGGAGGGGGCAUUCUAAU |
| | loop-symmetric_ | AUAUCUCUGAACUAAUCAUC |
| | AGAUUU-UUUGCG | |
| 74 | 5_1-1_mismatch_ | GAUAUUGUGACAUAUCUCUGCACUCAU |
| | A-C | CACACAAAAGAUGCCUGCACUCUAUAG |
| | 33_6-6_internal_ | GAUCCACAGGGAGGGGGCAUCUUAAU |
| | loop-symmetric_ | AUAUCUCUGAACUAAUCAUC |
| | AGAUUU-CUGCAC | |
| 75 | 33_6-6_internal_ | GAUAUUGUGACAUAUCUCUGCACUCAU |
| | loop-symmetric_ | CACACAAAAGAUGCCGCGGGUCUAUAG |
| | AGAUUU-CGCGGG | GAUCCACAGGGAGGGGGCAUUUUAAU |
| | | AUAUCUCUGAACUAAUCAUC |
| 76 | 3_1-1_mismatch_ | GAUAUUGUGACAUAUCUCUGCACUCAU |
| | A-C | CACACAAAAGAUGGUUUAAUUCUAUA |
| | 34_6-6_internal_ | GGAUCCACAGGGAGGGGGCAUUUCAA |
| | loop-symmetric_ | UAUAUCUCUGAACUAAUCAUC |
| | GAUUUG-GUUUAA | |
| 77 | 4_1-1_mismatch_ | GAUAUUGUGACAUAUCUCUGCACUCAU |
| | A-C | CACACAAAAGAUGGCUGGGUUCUAUA |
| | 34_6-6_internal_ | GGAUCCACAGGGAGGGGGCAUUCUAA |
| | loop-symmetric_ | UAUAUCUCUGAACUAAUCAUC |
| | GAUUUG-GCUGGG | |
| 78 | 5_1-1_mismatch_ | GAUAUUGUGACAUAUCUCUGCACUCAU |
| | A-C | CACACAAAAGAUGACUCCGUUCUAUAG |
| | 34_6-6_internal_ | GAUCCACAGGGAGGGGGCAUCUUAAU |
| | loop-symmetric_ | AUAUCUCUGAACUAAUCAUC |
| | GAUUUG-ACUCCG | |
| 79 | 34_6-6_internal_ | GAUAUUGUGACAUAUCUCUGCACUCAU |
| | loop-symmetric_ | CACACAAAAGAUGGUUACAUUCUAUA |
| | GAUUUG-GUUACA | GGAUCCACAGGGAGGGGGCAUUUUAA |
| | | UAUAUCUCUGAACUAAUCAUC |
| 80 | 0_1-1_mismatch_ | GAUAUUGUGACAUAUCUCUGCACUCAU |
| | A-C | CACACAAAAGAUCUGUCACUUCUAUAG |
| | 35_6-6_internal_ | GAUCCACAGGGAGGGGGCAUUUUAAC |
| | loop-symmetric_ | AUAUCUCUGAACUAAUCAUC |
| | AUUUGC-CUGUCA | |
| 81 | 3_1-1_mismatch_ | GAUAUUGUGACAUAUCUCUGCACUCAU |
| | A-C | CACACAAAAGAUCUUGAGCUUCUAUAG |
| | 35_6-6_internal_ | GAUCCACAGGGAGGGGGCAUUUCAAU |
| | loop-symmetric_ | AUAUCUCUGAACUAAUCAUC |
| | AUUUGC-CUUGAG | |
| 82 | 4_1-1_mismatch_ | GAUAUUGUGACAUAUCUCUGCACUCAU |
| | A-C | CACACAAAAGAUCUUAGGCUUCUAUAG |
| | 35_6-6_internal_ | GAUCCACAGGGAGGGGGCAUUCUAAU |
| | loop-symmetric_ | AUAUCUCUGAACUAAUCAUC |
| | AUUUGC-CUUAGG | |
| 83 | 5_1-1_mismatch_ | GAUAUUGUGACAUAUCUCUGCACUCAU |
| | A-C | CACACAAAAGAUAAGUGCCUUCUAUAG |
| | 35_6-6_internal_ | GAUCCACAGGGAGGGGGCAUCUUAAU |
| | loop-symmetric_ | AUAUCUCUGAACUAAUCAUC |
| | AUUUGC-AAGUGC | |
| 84 | 35_6-6_internal_ | GAUAUUGUGACAUAUCUCUGCACUCAU |
| | loop-symmetric_ | CACACAAAAGAUCUUCCACUUCUAUAG |
| | AUUUGC-CUUCCA | GAUCCACAGGGAGGGGGCAUUUUAAU |
| | | AUAUCUCUGAACUAAUCAUC |
| 85 | 0_1-1_mismatch_ | GAUAUUGUGACAUAUCUCUGCACUCAU |
| | A-C | CACACAAAAGACAACUUUCUUCUAUAG |
| | 36_6-6_internal_ | GAUCCACAGGGAGGGGGCAUUUUAAC |
| | loop-symmetric_ | AUAUCUCUGAACUAAUCAUC |
| | UUUGCA-CAACUU | |
| 86 | 3_1-1_mismatch_ | GAUAUUGUGACAUAUCUCUGCACUCAU |
| | A-C | CACACAAAAGACCACUCUCUUCUAUAG |
| | 36_6-6_internal_ | GAUCCACAGGGAGGGGGCAUUUCAAU |
| | loop-symmetric_ | AUAUCUCUGAACUAAUCAUC |
| | UUUGCA-CCACUC | |
| 87 | 4_1-1_mismatch_ | GAUAUUGUGACAUAUCUCUGCACUCAU |
| | A-C | CACACAAAAGAACGGUUUCUUCUAUAG |
| | 36_6-6_internal_ | GAUCCACAGGGAGGGGGCAUUCUAAU |
| | loop-symmetric_ | AUAUCUCUGAACUAAUCAUC |
| | UUUGCA-ACGGUU | |
| 88 | 5_1-1_mismatch_ | GAUAUUGUGACAUAUCUCUGCACUCAU |
| | A-C | CACACAAAAGACACCUCUCUUCUAUAG |
| | 36_6-6_internal_ | GAUCCACAGGGAGGGGGCAUCUUAAU |
| | loop-symmetric_ | AUAUCUCUGAACUAAUCAUC |
| | UUUGCA-CACCUC | |
| 89 | 36_6-6_internal_ | GAUAUUGUGACAUAUCUCUGCACUCAU |
| | loop-symmetric_ | CACACAAAAGACUAGUUUCUUCUAUAG |
| | UUUGCA-CUAGUU | GAUCCACAGGGAGGGGGCAUUUUAAU |
| | | AUAUCUCUGAACUAAUCAUC |
| 90 | 0_1-1_mismatch_ | GAUAUUGUGACAUAUCUCUGCACUCAU |
| | A-C | CACACAAAAGCGAUCCAUCUUCUAUAG |
| | 37_6-6_internal_ | GAUCCACAGGGAGGGGGCAUUUUAAC |
| | loop-symmetric_ | AUAUCUCUGAACUAAUCAUC |
| | UUGCAU-CGAUCC | |
| 91 | 3_1-1_mismatch_ | GAUAUUGUGACAUAUCUCUGCACUCAU |
| | A-C | CACACAAAAGCACGUCAUCUUCUAUAG |
| | 37_6-6_internal_ | GAUCCACAGGGAGGGGGCAUUUCAAU |
| | loop-symmetric_ | AUAUCUCUGAACUAAUCAUC |
| | UUGCAU-CACGUC | |
| 92 | 4_1-1_mismatch_ | GAUAUUGUGACAUAUCUCUGCACUCAU |
| | A-C | CACACAAAAGCUAGUUAUCUUCUAUAG |
| | 37_6-6_internal_ | GAUCCACAGGGAGGGGGCAUUCUAAU |
| | loop-symmetric_ | AUAUCUCUGAACUAAUCAUC |
| | UUGCAU-CUAGUU | |
| 93 | 5_1-1_mismatch_ | GAUAUUGUGACAUAUCUCUGCACUCAU |
| | A-C | CACACAAAAGCGUUUUAUCUUCUAUAG |
| | 37_6-6_internal_ | GAUCCACAGGGAGGGGGCAUCUUAAU |
| | loop-symmetric_ | AUAUCUCUGAACUAAUCAUC |
| | UUGCAU-CGUUUU | |
| 94 | 37_6-6_internal_ | GAUAUUGUGACAUAUCUCUGCACUCAU |
| | loop-symmetric_ | CACACAAAAGUCUGAUAUCUUCUAUAG |
| | UUGCAU-UCUGAU | GAUCCACAGGGAGGGGGCAUUUUAAU |
| | | AUAUCUCUGAACUAAUCAUC |
| 95 | 0_1-1_mismatch_ | GAUAUUGUGACAUAUCUCUGCACUCAU |
| | A-C | CACACAAAACUUAUUAAUCUUCUAUAG |
| | 38_6-6_internal_ | GAUCCACAGGGAGGGGGCAUUUUAAC |
| | loop-symmetric_ | AUAUCUCUGAACUAAUCAUC |
| | UGCAUC-CUUAUU | |
| 96 | 3_1-1_mismatch_ | GAUAUUGUGACAUAUCUCUGCACUCAU |
| | A-C | CACACAAAAUUUAUCAAUCUUCUAUAG |
| | 38_6-6_internal_ | GAUCCACAGGGAGGGGGCAUUUCAAU |
| | loop-symmetric_ | AUAUCUCUGAACUAAUCAUC |
| | UGCAUC-UUUAUC | |
| 97 | 4_1-1_mismatch_ | GAUAUUGUGACAUAUCUCUGCACUCAU |
| | A-C | CACACAAAAUUAAACAAUCUUCUAUAG |
| | 38_6-6_internal_ | GAUCCACAGGGAGGGGGCAUUCUAAU |
| | loop-symmetric_ | AUAUCUCUGAACUAAUCAUC |
| | UGCAUC-UUAAAC | |
| 98 | 38_6-6_internal_ | GAUAUUGUGACAUAUCUCUGCACUCAU |
| | loop-symmetric_ | CACACAAAAUUACGUAAUCUUCUAUAG |
| | UGCAUC-UUACGU | GAUCCACAGGGAGGGGGCAUUUUAAU |
| | | AUAUCUCUGAACUAAUCAUC |
| 99 | 0_1-1_mismatch_ | GAUAUUGUGACAUAUCUCUGCACUCAU |
| | A-C | CACACAAAUCUAUAAAAUCUUCUAUAG |
| | 39_6-6_internal_ | GAUCCACAGGGAGGGGGCAUUUUAAC |
| | loop-symmetric_ | AUAUCUCUGAACUAAUCAUC |
| | GCAUCU-UCUAUA | |
| 100 | 3_1-1_mismatch_ | GAUAUUGUGACAUAUCUCUGCACUCAU |
| | A-C | CACACAAAUACGGAAAAUCUUCUAUAG |
| | 39_6-6_internal_ | GAUCCACAGGGAGGGGGCAUUUCAAU |
| | loop-symmetric_ | AUAUCUCUGAACUAAUCAUC |
| | GCAUCU-UACGGA | |
| 101 | 4_1-1_mismatch_ | GAUAUUGUGACAUAUCUCUGCACUCAU |
| | A-C | CACACAAACUCCAGAAAUCUUCUAUAG |
| | 39_6-6_internal_ | GAUCCACAGGGAGGGGGCAUUCUAAU |
| | loop-symmetric_ | AUAUCUCUGAACUAAUCAUC |
| | GCAUCU-CUCCAG | |
| 102 | 5_1-1_mismatch_ | GAUAUUGUGACAUAUCUCUGCACUCAU |
| | A-C | CACACAAAUCAACAAAAUCUUCUAUAG |
| | 39_6-6_internal_ | GAUCCACAGGGAGGGGGCAUCUUAAU |
| | loop-symmetric_ | AUAUCUCUGAACUAAUCAUC |
| | GCAUCU-UCAACA | |
| 103 | 39_6-6_internal_ | GAUAUUGUGACAUAUCUCUGCACUCAU |
| | loop-symmetric_ | CACACAAACAUACGAAAUCUUCUAUAG |
| | GCAUCU-CAUACG | GAUCCACAGGGAGGGGGCAUUUUAAU |
| | | AUAUCUCUGAACUAAUCAUC |
| 104 | 0_1-1_mismatch_ | GAUAUUGUGACAUAUCUCUGCACUCAU |
| | A-C | CACACAAUAUCGUCAAAUCUUCUAUAG |
| | 40_6-6_internal_ | GAUCCACAGGGAGGGGGCAUUUUAAC |
| | loop-symmetric_ | AUAUCUCUGAACUAAUCAUC |
| | CAUCUU-UAUCGU | |
| 105 | 3_1-1_mismatch_ | GAUAUUGUGACAUAUCUCUGCACUCAU |
| | A-C | CACACAAUCCGCACAAAUCUUCUAUAG |
| | 40_6-6_internal_ | GAUCCACAGGGAGGGGGCAUUUCAAU |
| | loop-symmetric_ | AUAUCUCUGAACUAAUCAUC |
| | CAUCUU-UCCGCA | |
| 106 | 4_1-1_mismatch_ | GAUAUUGUGACAUAUCUCUGCACUCAU |
| | A-C | CACACAACUUGACCAAAUCUUCUAUAG |
| | 40_6-6_internal_ | GAUCCACAGGGAGGGGGCAUUCUAAU |
| | loop-symmetric_ | AUAUCUCUGAACUAAUCAUC |
| | CAUCUU-CUUGAC | |
| 107 | 5_1-1_mismatch_ | GAUAUUGUGACAUAUCUCUGCACUCAU |
| | A-C | CACACAAUCUUUCCAAAUCUUCUAUAG |
| | 40_6-6_internal_ | GAUCCACAGGGAGGGGGCAUCUUAAU |
| | loop-symmetric_ | AUAUCUCUGAACUAAUCAUC |
| | CAUCUU-UCUUUC | |
| 108 | 40_6-6_internal_ | GAUAUUGUGACAUAUCUCUGCACUCAU |
| | loop-symmetric_ | CACACAACGCGACCAAAUCUUCUAUAG |
| | CAUCUU-CGCGAC | GAUCCACAGGGAGGGGGCAUUUUAAU |
| | | AUAUCUCUGAACUAAUCAUC |
| 109 | 0_1-1_mismatch_ | GAUAUUGUGACAUAUCUCUGCACUCAU |
| | A-C | CACACAUCUAUGGCAAAUCUUCUAUAG |
| | 41_6-6_internal_ | GAUCCACAGGGAGGGGGCAUUUUAAC |
| | loop-symmetric_ | AUAUCUCUGAACUAAUCAUC |
| | AUCUUU-UCUAUG | |
| 110 | 3_1-1_mismatch_ | GAUAUUGUGACAUAUCUCUGCACUCAU |
| | A-C | CACACACGCGCCGCAAAUCUUCUAUAG |
| | 41_6-6_internal_ | GAUCCACAGGGAGGGGGCAUUUCAAU |
| | loop-symmetric_ | AUAUCUCUGAACUAAUCAUC |
| | AUCUUU-CGCGCC | |
| 111 | 4_1-1_mismatch_ | GAUAUUGUGACAUAUCUCUGCACUCAU |
| | A-C | CACACAUGUAUCGCAAAUCUUCUAUAG |
| | 41_6-6_internal_ | GAUCCACAGGGAGGGGGCAUUCUAAU |
| | loop-symmetric_ | AUAUCUCUGAACUAAUCAUC |
| | AUCUUU-UGUAUC | |
| 112 | 5_1-1_mismatch_ | GAUAUUGUGACAUAUCUCUGCACUCAU |
| | A-C | CACACAUUGCUCGCAAAUCUUCUAUAG |
| | 41_6-6_internal_ | GAUCCACAGGGAGGGGGCAUCUUAAU |
| | loop-symmetric_ | AUAUCUCUGAACUAAUCAUC |
| | AUCUUU-UUGCUC | |
| 113 | 41_6-6_internal_ | GAUAUUGUGACAUAUCUCUGCACUCAU |
| | loop-symmetric_ | CACACACCGCCCGCAAAUCUUCUAUAG |
| | AUCUUU-CCGCCC | GAUCCACAGGGAGGGGGCAUUUUAAU |
| | | AUAUCUCUGAACUAAUCAUC |
| 114 | 0_1-1_mismatch_ | GAUAUUGUGACAUAUCUCUGCACUCAU |
| | A-C | CACACUUCUUCUGCAAAUCUUCUAUAG |
| | 42_6-6_internal_ | GAUCCACAGGGAGGGGGCAUUUUAAC |
| | loop-symmetric_ | AUAUCUCUGAACUAAUCAUC |
| | UCUUUU-UUCUUC | |
| 115 | 3_1-1_mismatch_ | GAUAUUGUGACAUAUCUCUGCACUCAU |
| | A-C | CACACCUGCUCUGCAAAUCUUCUAUAG |
| | 42_6-6_internal_ | GAUCCACAGGGAGGGGGCAUUUCAAU |
| | loop-symmetric_ | AUAUCUCUGAACUAAUCAUC |
| | UCUUUU-CUGCUC | |
| 116 | 4_1-1_mismatch_ | GAUAUUGUGACAUAUCUCUGCACUCAU |
| | A-C | CACACCUUGUUUGCAAAUCUUCUAUAG |
| | 42_6-6_internal_ | GAUCCACAGGGAGGGGGCAUUCUAAU |
| | loop-symmetric_ | AUAUCUCUGAACUAAUCAUC |
| | UCUUUU-CUUGUU | |
| 117 | 42_6-6_internal_ | GAUAUUGUGACAUAUCUCUGCACUCAU |
| | loop-symmetric_ | CACACCUAUAUUGCAAAUCUUCUAUAG |
| | UCUUUU-CUAUAU | GAUCCACAGGGAGGGGGCAUUUUAAU |
| | | AUAUCUCUGAACUAAUCAUC |
| 118 | 0_1-1_mismatch_ | GAUAUUGUGACAUAUCUCUGCACUCAU |
| | A-C | CACAGUUGUAAUGCAAAUCUUCUAUA |
| | 43_6-6_internal_ | GGAUCCACAGGGAGGGGGCAUUUUAA |
| | loop-symmetric_ | CAUAUCUCUGAACUAAUCAUC |
| | CUUUUG-GUUGUA | |
| 119 | 4_1-1_mismatch_ | GAUAUUGUGACAUAUCUCUGCACUCAU |
| | A-C | CACAACCUGAAUGCAAAUCUUCUAUAG |
| | 43_6-6_internal_ | GAUCCACAGGGAGGGGGCAUUCUAAU |
| | loop-symmetric_ | AUAUCUCUGAACUAAUCAUC |
| | CUUUUG-ACCUGA | |
| 120 | 5_1-1_mismatch_ | GAUAUUGUGACAUAUCUCUGCACUCAU |
| | A-C | CACAGCUGUCAUGCAAAUCUUCUAUAG |
| | 43_6-6_internal_ | GAUCCACAGGGAGGGGGCAUCUUAAU |
| | loop-symmetric_ | AUAUCUCUGAACUAAUCAUC |
| | CUUUUG-GCUGUC | |
| 121 | 43_6-6_internal_ | GAUAUUGUGACAUAUCUCUGCACUCAU |
| | loop-symmetric_ | CACAAGACCAAUGCAAAUCUUCUAUAG |
| | CUUUUG-AGACCA | GAUCCACAGGGAGGGGGCAUUUUAAU |
| | | AUAUCUCUGAACUAAUCAUC |
| 122 | 0_1-1_mismatch_ | GAUAUUGUGACAUAUCUCUGCACUCAU |
| | A-C | CACUACCUCGAUGCAAAUCUUCUAUAG |
| | 44_6-6_internal_ | GAUCCACAGGGAGGGGGCAUUUUAAC |
| | loop-symmetric_ | AUAUCUCUGAACUAAUCAUC |
| | UUUUGU-UACCUC | |
| 123 | 3_1-1_mismatch_ | GAUAUUGUGACAUAUCUCUGCACUCAU |
| | A-C | CACCUUUCCGAUGCAAAUCUUCUAUAG |
| | 44_6-6_internal_ | GAUCCACAGGGAGGGGGCAUUUCAAU |
| | loop-symmetric_ | AUAUCUCUGAACUAAUCAUC |
| | UUUUGU-CUUUCC | |
| 124 | 4_1-1_mismatch_ | GAUAUUGUGACAUAUCUCUGCACUCAU |
| | A-C | CACUUGCCUGAUGCAAAUCUUCUAUAG |
| | 44_6-6_internal_ | GAUCCACAGGGAGGGGGCAUUCUAAU |
| | loop-symmetric_ | AUAUCUCUGAACUAAUCAUC |
| | UUUUGU-UUGCCU | |
| 125 | 5_1-1_mismatch_ | GAUAUUGUGACAUAUCUCUGCACUCAU |
| | A-C | CACUGUUUCGAUGCAAAUCUUCUAUAG |
| | 44_6-6_internal_ | GAUCCACAGGGAGGGGGCAUCUUAAU |
| | loop-symmetric_ | AUAUCUCUGAACUAAUCAUC |
| | UUUUGU-UGUUUC | |
| 126 | 44_6-6_internal_ | GAUAUUGUGACAUAUCUCUGCACUCAU |
| | loop-symmetric_ | CACCUUGCCGAUGCAAAUCUUCUAUAG |
| | UUUUGU-CUUGCC | GAUCCACAGGGAGGGGGCAUUUUAAU |
| | | AUAUCUCUGAACUAAUCAUC |
| 127 | -12_6-6_internal_ | GAUAUUGUGACAUAUCUCUGCACUCAU |
| | loop-symmetric_ | CACACAAAAGAUGCAAAUCUUCUAUAG |
| | GAUUAG-AAACGA | GAUCCACAGGGAGGGGGCAUUUUAAC |
| | 0_1-1_mismatch_ | AUAUCUCUGAAAAACGAAUC |
| | A-C | |
| 128 | -12_6-6_internal_ | GAUAUUGUGACAUAUCUCUGCACUCAU |
| | loop-symmetric_ | CACACAAAAGAUGCAAAUCUUCUAUAG |
| | GAUUAG-GAAGAG | GAUCCACAGGGAGGGGGCAUUUCAAU |
| | 3_1-1_mismatch_ | AUAUCUCUGAAGAAGAGAUC |
| | A-C | |
| 129 | -12_6-6_internal_ | GAUAUUGUGACAUAUCUCUGCACUCAU |
| | loop-symmetric_ | CACACAAAAGAUGCAAAUCUUCUAUAG |
| | GAUUAG-GCACGA | GAUCCACAGGGAGGGGGCAUUCUAAU |
| | 4_1-1_mismatch_ | AUAUCUCUGAAGCACGAAUC |
| | A-C | |
| 130 | -12_6-6_internal_ | GAUAUUGUGACAUAUCUCUGCACUCAU |
| | loop-symmetric_ | CACACAAAAGAUGCAAAUCUUCUAUAG |
| | GAUUAG-AGAUAA | GAUCCACAGGGAGGGGGCAUCUUAAU |
| | 5_1-1_mismatch_ | AUAUCUCUGAAAGAUAAAUC |
| | A-C | |
| 131 | -12_6-6_internal_ | GAUAUUGUGACAUAUCUCUGCACUCAU |
| | loop-symmetric_ | CACACAAAAGAUGCAAAUCUUCUAUAG |
| | GAUUAG-ACGCCA | GAUCCACAGGGAGGGGGCAUUUUAAU |
| | | AUAUCUCUGAAACGCCAAUC |
| 132 | -12_6-6_internal_ | GAUAUUGUGACAUAUCUCUGCACUCAU |
| | loop-symmetric_ | CACACAAAAGAUGCAAAUCUUCUAUAG |
| | GAUUAG-AAACCG | CAGGAUCAGGGAGGGGGCAUUUUAAC |
| | 0_1-1_mismatch_ | AUAUCUCUGAAAAACCGAUC |
| | A-C | |
| | 20_6-6_internal_ | |
| | loop-symmetric_ | |
| | UGGAUC-CAGGAU | |
| 133 | -12_6-6_internal_ | GAUAUUGUGACAUAUCUCUGCACUCAU |
| | loop-symmetric_ | CACACAAAAGAUGCAAAUCUUCUAUAG |
| | GAUUAG-AGCGGG | CCUAAUCAGGGAGGGGGCAUUUCAAU |
| | 3_1-1_mismatch_ | AUAUCUCUGAAAGCGGGAUC |
| | A-C | |
| | 20_6-6_internal_ | |
| | loop-symmetric_ | |
| | UGGAUC-CCUAAU | |
| 134 | -12_6-6_internal_ | GAUAUUGUGACAUAUCUCUGCACUCAU |
| | loop-symmetric_ | CACACAAAAGAUGCAAAUCUUCUAUAG |
| | GAUUAG-GCCUAA | CACAGUCAGGGAGGGGGCAUUCUAAU |
| | 4_1-1_mismatch_ | AUAUCUCUGAAGCCUAAAUC |
| | A-C | |
| | 20_6-6_internal_ | |
| | loop-symmetric_ | |
| | UGGAUC-CACAGU | |
| 135 | -12_6-6_internal_ | GAUAUUGUGACAUAUCUCUGCACUCAU |
| | loop-symmetric_ | CACACAAAAGAUGCAAAUCUUCUAUAG |
| | GAUUAG-GCCAUA | UUAAAUCAGGGAGGGGGCAUCUUAAU |
| | 5_1-1_mismatch_ | AUAUCUCUGAAGCCAUAAUC |
| | A-C | |
| | 20_6-6_internal_ | |
| | loop-symmetric_ | |
| | UGGAUC-UUAAAU | |
| 136 | -12_6-6_internal_ | GAUAUUGUGACAUAUCUCUGCACUCAU |
| | loop-symmetric_ | CACACAAAAGAUGCAAAUCUUCUAUAG |
| | GAUUAG-GGCCAA | CCGUGUCAGGGAGGGGGCAUUUUAAU |
| | 20_6-6_internal_ | AUAUCUCUGAAGGCCAAAUC |
| | loop-symmetric_ | |
| | UGGAUC-CCGUGU | |
| 137 | -12_6-6_internal_ | GAUAUUGUGACAUAUCUCUGCACUCAU |
| | loop-symmetric_ | CACACAAAAGAUGCAAAUCUUCUAUAU |
| | GAUUAG-AGAUAG | ACCGAACAGGGAGGGGGCAUUUUAAC |
| | 0_1-1_mismatch_ | AUAUCUCUGAAAGAUAGAUC |
| | A-C | |
| | 21_6-6_internal_ | |
| | loop-symmetric_ | |
| | GGAUCC-UACCGA | |
| 138 | -12_6-6_internal_ | GAUAUUGUGACAUAUCUCUGCACUCAU |
| | loop-symmetric_ | CACACAAAAGAUGCAAAUCUUCUAUAC |
| | GAUUAG-AUUUAG | ACGCAACAGGGAGGGGGCAUUCUAAU |
| | 4_1-1_mismatch_ | AUAUCUCUGAAAUUUAGAUC |
| | A-C | |
| | 21_6-6_internal_ | |
| | loop-symmetric_ | |
| | GGAUCC-CACGCA | |
| 139 | -12_6-6_internal_ | GAUAUUGUGACAUAUCUCUGCACUCAU |
| | loop-symmetric_ | CACACAAAAGAUGCAAAUCUUCUAUAA |
| | GAUUAG-GGGGCA | AAGCAACAGGGAGGGGGCAUCUUAAU |
| | 5_1-1_mismatch_ | AUAUCUCUGAAGGGGCAAUC |
| | A-C | |
| | 21_6-6_internal_ | |
| | loop-symmetric_ | |
| | GGAUCC-AAAGCA | |
| 140 | -12_6-6_internal_ | GAUAUUGUGACAUAUCUCUGCACUCAU |
| | loop-symmetric_ | CACACAAAAGAUGCAAAUCUUCUAUAA |
| | GAUUAG-GGGUAA | ACGAGACAGGGAGGGGGCAUUUUAAU |
| | 21_6-6_internal_ | AUAUCUCUGAAGGGUAAAUC |
| | loop-symmetric_ | |
| | GGAUCC-AACGAG | |
| 141 | -12_6-6_internal_ | GAUAUUGUGACAUAUCUCUGCACUCAU |
| | loop-symmetric_ | CACACAAAAGAUGCAAAUCUUCUAUCC |
| | GAUUAG-GGAAAA | UCAACACAGGGAGGGGGCAUUUUAAC |
| | 0_1-1_mismatch_ | AUAUCUCUGAAGGAAAAAUC |
| | A-C | |
| | 22_6-6_internal_ | |
| | loop-symmetric_ | |
| | GAUCCU-CCUCAA | |
| 142 | -12_6-6_internal_ | GAUAUUGUGACAUAUCUCUGCACUCAU |
| | loop-symmetric_ | CACACAAAAGAUGCAAAUCUUCUAUCG |
| | GAUUAG-GGUGGG | CUGGCACAGGGAGGGGGCAUUUCAAU |
| | 3_1-1_mismatch_ | AUAUCUCUGAAGGUGGGAUC |
| | A-C | |
| | 22_6-6_internal_ | |
| | loop-symmetric_ | |
| | GAUCCU-CGCUGG | |
| 143 | -12_6-6_internal_ | GAUAUUGUGACAUAUCUCUGCACUCAU |
| | loop-symmetric_ | CACACAAAAGAUGCAAAUCUUCUAUCC |
| | GAUUAG-GGCACA | AGCACACAGGGAGGGGGCAUUCUAAU |
| | 4_1-1_mismatch_ | AUAUCUCUGAAGGCACAAUC |
| | A-C | |
| | 22_6-6_internal_ | |
| | loop-symmetric_ | |
| | GAUCCU-CCAGCA | |
| 144 | -12_6-6_internal_ | GAUAUUGUGACAUAUCUCUGCACUCAU |
| | loop-symmetric_ | CACACAAAAGAUGCAAAUCUUCUAUCU |
| | GAUUAG-AUCGAA | UGUACACAGGGAGGGGGCAUCUUAAU |
| | 5_1-1_mismatch_ | AUAUCUCUGAAAUCGAAAUC |
| | A-C | |
| | 22_6-6_internal_ | |
| | loop-symmetric_ | |
| | GAUCCU-CUUGUA | |
| 145 | -12_6-6_internal_ | GAUAUUGUGACAUAUCUCUGCACUCAU |
| | loop-symmetric_ | CACACAAAAGAUGCAAAUCUUCUAUCC |
| | GAUUAG-GAUAGA | AGAACACAGGGAGGGGGCAUUUUAAU |
| | 22_6-6_internal_ | AUAUCUCUGAAGAUAGAAUC |
| | loop-symmetric_ | |
| | GAUCCU-CCAGAA | |
| 146 | -12_6-6_internal_ | GAUAUUGUGACAUAUCUCUGCACUCAU |
| | loop-symmetric_ | CACACAAAAGAUGCAAAUCUUCUAGAA |
| | GAUUAG-AGGGGG | AGCCCACAGGGAGGGGGCAUUUUAACA |
| | 0_1-1_mismatch_ | UAUCUCUGAAAGGGGGAUC |
| | A-C | |
| | 23_6-6_internal_ | |
| | loop-symmetric_ | |
| | AUCCUA-GAAAGC | |
| 147 | -12_6-6_internal_ | GAUAUUGUGACAUAUCUCUGCACUCAU |
| | loop-symmetric_ | CACACAAAAGAUGCAAAUCUUCUACUC |
| | GAUUAG-GCACGG | CUGCCACAGGGAGGGGGCAUUUCAAUA |
| | 3_1-1_mismatch_ | UAUCUCUGAAGCACGGAUC |
| | A-C | |
| | 23_6-6_internal_ | |
| | loop-symmetric_ | |
| | AUCCUA-CUCCUG | |
| 148 | -12_6-6_internal_ | GAUAUUGUGACAUAUCUCUGCACUCAU |
| | loop-symmetric_ | CACACAAAAGAUGCAAAUCUUCUACCU |
| | GAUUAG-ACCGUA | AACCCACAGGGAGGGGGCAUUCUAAUA |
| | 4_1-1_mismatch_ | UAUCUCUGAAACCGUAAUC |
| | A-C | |
| | 23_6-6_internal_ | |
| | loop-symmetric_ | |
| | AUCCUA-CCUAAC | |
| 149 | -12_6-6_internal_ | GAUAUUGUGACAUAUCUCUGCACUCAU |
| | loop-symmetric_ | CACACAAAAGAUGCAAAUCUUCUAAUC |
| | GAUUAG-AAGCAG | AUCCCACAGGGAGGGGGCAUCUUAAUA |
| | 5_1-1_mismatch_ | UAUCUCUGAAAAGCAGAUC |
| | A-C | |
| | 23_6-6_internal_ | |
| | loop-symmetric_ | |
| | AUCCUA-AUCAUC | |
| 150 | -12_6-6_internal_ | GAUAUUGUGACAUAUCUCUGCACUCAU |
| | loop-symmetric_ | CACACAAAAGAUGCAAAUCUUCUAGCG |
| | GAUUAG-GAGUGG | AAACCACAGGGAGGGGGCAUUUUAAU |
| | 23_6-6_internal_ | AUAUCUCUGAAGAGUGGAUC |
| | loop-symmetric_ | |
| | AUCCUA-GCGAAA | |
| 151 | -12_6-6_internal_ | GAUAUUGUGACAUAUCUCUGCACUCAU |
| | loop-symmetric_ | CACACAAAAGAUGCAAAUCUUCUUCUA |
| | GAUUAG-AUCUGG | CUUCCACAGGGAGGGGGCAUUUUAACA |
| | 0_1-1_mismatch_ | UAUCUCUGAAAUCUGGAUC |
| | A-C | |
| | 24_6-6_internal_ | |
| | loop-symmetric_ | |
| | UCCUAU-UCUACU | |
| 152 | -12_6-6_internal_ | GAUAUUGUGACAUAUCUCUGCACUCAU |
| | loop-symmetric_ | CACACAAAAGAUGCAAAUCUUCUUGCC |
| | GAUUAG-GGUACA | UUUCCACAGGGAGGGGGCAUUUCAAU |
| | 3_1-1_mismatch_ | AUAUCUCUGAAGGUACAAUC |
| | A-C | |
| | 24_6-6_internal_ | |
| | loop-symmetric_ | |
| | UCCUAU-UGCCUU | |
| 153 | -12_6-6_internal_ | GAUAUUGUGACAUAUCUCUGCACUCAU |
| | loop-symmetric_ | CACACAAAAGAUGCAAAUCUUCUUACC |
| | GAUUAG-GGAGCG | AUUCCACAGGGAGGGGGCAUUCUAAU |
| | 4_1-1_mismatch_ | AUAUCUCUGAAGGAGCGAUC |
| | A-C | |
| | 24_6-6_internal_ | |
| | loop-symmetric_ | |
| | UCCUAU-UACCAU | |
| 154 | -12_6-6_internal_ | GAUAUUGUGACAUAUCUCUGCACUCAU |
| | loop-symmetric_ | CACACAAAAGAUGCAAAUCUUCUUCAC |
| | GAUUAG-GGCCGG | CUUCCACAGGGAGGGGGCAUCUUAAUA |
| | 5_1-1_mismatch_ | UAUCUCUGAAGGCCGGAUC |
| | A-C | |
| | 24_6-6_internal_ | |
| | loop-symmetric_ | |
| | UCCUAU-UCACCU | |
| 155 | -12_6-6_internal_ | GAUAUUGUGACAUAUCUCUGCACUCAU |
| | loop-symmetric_ | CACACAAAAGAUGCAAAUCUUCUUCAC |
| | GAUUAG-GGCAGA | CCUCCACAGGGAGGGGGCAUUUUAAUA |
| | 24_6-6_internal_ | UAUCUCUGAAGGCAGAAUC |
| | loop-symmetric_ | |
| | UCCUAU-UCACCC | |
| 156 | -12_6-6_internal_ | GAUAUUGUGACAUAUCUCUGCACUCAU |
| | loop-symmetric_ | CACACAAAAGAUGCAAAUCUUCGAAAC |
| | GAUUAG-GACAGA | CAUCCACAGGGAGGGGGCAUUUUAACA |
| | 0_1-1_mismatch_ | UAUCUCUGAAGACAGAAUC |
| | A-C | |
| | 25_6-6_internal_ | |
| | loop-symmetric_ | |
| | CCUAUA-GAAACC | |
| 157 | -12_6-6_internal_ | GAUAUUGUGACAUAUCUCUGCACUCAU |
| | loop-symmetric_ | CACACAAAAGAUGCAAAUCUUCACAAA |
| | GAUUAG-AGCGAG | UAUCCACAGGGAGGGGGCAUUUCAAU |
| | 3_1-1_mismatch_ | AUAUCUCUGAAAGCGAGAUC |
| | A-C | |
| | 25_6-6_internal_ | |
| | loop-symmetric_ | |
| | CCUAUA-ACAAAU | |
| 158 | -12_6-6_internal_ | GAUAUUGUGACAUAUCUCUGCACUCAU |
| | loop-symmetric_ | CACACAAAAGAUGCAAAUCUUCCCCCU |
| | GAUUAG-GGGUGG | UAUCCACAGGGAGGGGGCAUUCUAAU |
| | 4_1-1_mismatch_ | AUAUCUCUGAAGGGUGGAUC |
| | A-C | |
| | 25_6-6_internal_ | |
| | loop-symmetric_ | |
| | CCUAUA-CCCCUU | |
| 159 | -12_6-6_internal_ | GAUAUUGUGACAUAUCUCUGCACUCAU |
| | loop-symmetric_ | CACACAAAAGAUGCAAAUCUUCACCCC |
| | GAUUAG-GACGCG | UAUCCACAGGGAGGGGGCAUCUUAAU |
| | 5_1-1_mismatch_ | AUAUCUCUGAAGACGCGAUC |
| | A-C | |
| | 25_6-6_internal_ | |
| | loop-symmetric_ | |
| | CCUAUA-ACCCCU | |
| 160 | -12_6-6_internal_ | GAUAUUGUGACAUAUCUCUGCACUCAU |
| | loop-symmetric_ | CACACAAAAGAUGCAAAUCUUCGCACC |
| | GAUUAG-GAGCCG | AAUCCACAGGGAGGGGGCAUUUUAAU |
| | 25_6-6_internal_ | AUAUCUCUGAAGAGCCGAUC |
| | loop-symmetric_ | |
| | CCUAUA-GCACCA | |
| 161 | -12_6-6_internal_ | GAUAUUGUGACAUAUCUCUGCACUCAU |
| | loop-symmetric_ | CACACAAAAGAUGCAAAUCUUAUCGAC |
| | GAUUAG-ACAUGA | GAUCCACAGGGAGGGGGCAUUUUAAC |
| | 0_1-1_mismatch_ | AUAUCUCUGAAACAUGAAUC |
| | A-C | |
| | 26_6-6_internal_ | |
| | loop-symmetric_ | |
| | CUAUAG-AUCGAC | |
| 162 | -12_6-6_internal_ | GAUAUUGUGACAUAUCUCUGCACUCAU |
| | loop-symmetric_ | CACACAAAAGAUGCAAAUCUUGCCCUU |
| | GAUUAG-ACUCGA | GAUCCACAGGGAGGGGGCAUUUCAAU |
| | 3_1-1_mismatch_ | AUAUCUCUGAAACUCGAAUC |
| | A-C | |
| | 26_6-6_internal_ | |
| | loop-symmetric_ | |
| | CUAUAG-GCCCUU | |
| 163 | -12_6-6_internal_ | GAUAUUGUGACAUAUCUCUGCACUCAU |
| | loop-symmetric_ | CACACAAAAGAUGCAAAUCUUGCUCGU |
| | GAUUAG-AGCAAG | GAUCCACAGGGAGGGGGCAUUCUAAU |
| | 4_1-1_mismatch_ | AUAUCUCUGAAAGCAAGAUC |
| | A-C | |
| | 26_6-6_internal_ | |
| | loop-symmetric_ | |
| | CUAUAG-GCUCGU | |
| 164 | -12_6-6_internal_ | GAUAUUGUGACAUAUCUCUGCACUCAU |
| | loop-symmetric_ | CACACAAAAGAUGCAAAUCUUGGUGA |
| | GAUUAG-GCCAGA | AGAUCCACAGGGAGGGGGCAUCUUAA |
| | 5_1-1_mismatch_ | UAUAUCUCUGAAGCCAGAAUC |
| | A-C | |
| | 26_6-6_internal_ | |
| | loop-symmetric_ | |
| | CUAUAG-GGUGAA | |
| 165 | -12_6-6_internal_ | GAUAUUGUGACAUAUCUCUGCACUCAU |
| | loop-symmetric_ | CACACAAAAGAUGCAAAUCUUGCGGUC |
| | GAUUAG-GACCGG | GAUCCACAGGGAGGGGGCAUUUUAAU |
| | 26_6-6_internal_ | AUAUCUCUGAAGACCGGAUC |
| | loop-symmetric_ | |
| | CUAUAG-GCGGUC | |
| 166 | -12_6-6_internal_ | GAUAUUGUGACAUAUCUCUGCACUCAU |
| | loop-symmetric_ | CACACAAAAGAUGCAAAUCUGACCUCG |
| | GAUUAG-AUCUGG | GAUCCACAGGGAGGGGGCAUUUCAAU |
| | 3_1-1_mismatch_ | AUAUCUCUGAAAUCUGGAUC |
| | A-C | |
| | 27_6-6_internal_ | |
| | loop-symmetric_ | |
| | UAUAGA-GACCUC | |
| 167 | -12_6-6_internal_ | GAUAUUGUGACAUAUCUCUGCACUCAU |
| | loop-symmetric_ | CACACAAAAGAUGCAAAUCUGAUUCUG |
| | GAUUAG-GGUUAA | GAUCCACAGGGAGGGGGCAUUCUAAU |
| | 4_1-1_mismatch_ | AUAUCUCUGAAGGUUAAAUC |
| | A-C | |
| | 27_6-6_internal_ | |
| | loop-symmetric_ | |
| | UAUAGA-GAUUCU | |
| 168 | -12_6-6_internal_ | GAUAUUGUGACAUAUCUCUGCACUCAU |
| | loop-symmetric_ | CACACAAAAGAUGCAAAUCUGCCUUUG |
| | GAUUAG-AGGCCG | GAUCCACAGGGAGGGGGCAUCUUAAU |
| | 5_1-1_mismatch_ | AUAUCUCUGAAAGGCCGAUC |
| | A-C | |
| | 27_6-6_internal_ | |
| | loop-symmetric_ | |
| | UAUAGA-GCCUUU | |
| 169 | -12_6-6_internal_ | GAUAUUGUGACAUAUCUCUGCACUCAU |
| | loop-symmetric_ | CACACAAAAGAUGCAAAUCUGGCCUCG |
| | GAUUAG-GCGUGA | GAUCCACAGGGAGGGGGCAUUUUAAU |
| | 27_6-6_internal_ | AUAUCUCUGAAGCGUGAAUC |
| | loop-symmetric_ | |
| | UAUAGA-GGCCUC | |
| 170 | -12_6-6_internal_ | GAUAUUGUGACAUAUCUCUGCACUCAU |
| | loop-symmetric_ | CACACAAAAGAUGCAAAUCGUAGUAA |
| | GAUUAG-GAAUAG | GGAUCCACAGGGAGGGGGCAUUUUAA |
| | 0_1-1_mismatch_ | CAUAUCUCUGAAGAAUAGAUC |
| | A-C | |
| | 28_6-6_internal_ | |
| | loop-symmetric_ | |
| | AUAGAA-GUAGUA | |
| 171 | -12_6-6_internal_ | GAUAUUGUGACAUAUCUCUGCACUCAU |
| | loop-symmetric_ | CACACAAAAGAUGCAAAUCGACACGAG |
| | GAUUAG-GCAUGG | GAUCCACAGGGAGGGGGCAUUUCAAU |
| | 3_1-1_mismatch_ | AUAUCUCUGAAGCAUGGAUC |
| | A-C | |
| | 28_6-6_internal_ | |
| | loop-symmetric_ | |
| | AUAGAA-GACACG | |
| 172 | -12_6-6_internal_ | GAUAUUGUGACAUAUCUCUGCACUCAU |
| | loop-symmetric_ | CACACAAAAGAUGCAAAUCGGGCGCAG |
| | GAUUAG-GAACGA | GAUCCACAGGGAGGGGGCAUUCUAAU |
| | 4_1-1_mismatch_ | AUAUCUCUGAAGAACGAAUC |
| | A-C | |
| | 28_6-6_internal_ | |
| | loop-symmetric_ | |
| | AUAGAA-GGGCGC | |
| 173 | -12_6-6_internal_ | GAUAUUGUGACAUAUCUCUGCACUCAU |
| | loop-symmetric_ | CACACAAAAGAUGCAAAUCACCACGAG |
| | GAUUAG-GGCAAA | GAUCCACAGGGAGGGGGCAUCUUAAU |
| | 5_1-1_mismatch_ | AUAUCUCUGAAGGCAAAAUC |
| | A-C | |
| | 28_6-6_internal_ | |
| | loop-symmetric_ | |
| | AUAGAA-ACCACG | |
| 174 | -12_6-6_internal_ | GAUAUUGUGACAUAUCUCUGCACUCAU |
| | loop-symmetric_ | CACACAAAAGAUGCAAAUCGUGAGCAG |
| | GAUUAG-AACGGG | GAUCCACAGGGAGGGGGCAUUUUAAU |
| | 28_6-6_internal_ | AUAUCUCUGAAAACGGGAUC |
| | loop-symmetric_ | |
| | AUAGAA-GUGAGC | |
| 175 | -12_6-6_internal_ | GAUAUUGUGACAUAUCUCUGCACUCAU |
| | loop-symmetric_ | CACACAAAAGAUGCAAAUGCAGGUUA |
| | GAUUAG-GCCUCG | GGAUCCACAGGGAGGGGGCAUUUUAA |
| | 0_1-1_mismatch_ | CAUAUCUCUGAAGCCUCGAUC |
| | A-C | |
| | 29_6-6_internal_ | |
| | loop-symmetric_ | |
| | UAGAAG-GCAGGU | |
| 176 | -12_6-6_internal_ | GAUAUUGUGACAUAUCUCUGCACUCAU |
| | loop-symmetric_ | CACACAAAAGAUGCAAAUAGCGUCUAG |
| | GAUUAG-GGGACA | GAUCCACAGGGAGGGGGCAUUUCAAU |
| | 3_1-1_mismatch_ | AUAUCUCUGAAGGGACAAUC |
| | A-C | |
| | 29_6-6_internal_ | |
| | loop-symmetric_ | |
| | UAGAAG-AGCGUC | |
| 177 | -12_6-6_internal_ | GAUAUUGUGACAUAUCUCUGCACUCAU |
| | loop-symmetric_ | CACACAAAAGAUGCAAAUAGCACCUAG |
| | GAUUAG-ACUCCA | GAUCCACAGGGAGGGGGCAUUCUAAU |
| | 4_1-1_mismatch_ | AUAUCUCUGAAACUCCAAUC |
| | A-C | |
| | 29_6-6_internal_ | |
| | loop-symmetric_ | |
| | UAGAAG-AGCACC | |
| 178 | -12_6-6_internal_ | GAUAUUGUGACAUAUCUCUGCACUCAU |
| | loop-symmetric_ | CACACAAAAGAUGCAAAUAAGAUUUA |
| | GAUUAG-GGAGCA | GGAUCCACAGGGAGGGGGCAUCUUAA |
| | 5_1-1_mismatch_ | UAUAUCUCUGAAGGAGCAAUC |
| | A-C | |
| | 29_6-6_internal_ | |
| | loop-symmetric_ | |
| | UAGAAG-AAGAUU | |
| 179 | -12_6-6_internal_ | GAUAUUGUGACAUAUCUCUGCACUCAU |
| | loop-symmetric_ | CACACAAAAGAUGCAAAUGACAGCUAG |
| | GAUUAG-GACCCG | GAUCCACAGGGAGGGGGCAUUUUAAU |
| | 29_6-6_internal_ | AUAUCUCUGAAGACCCGAUC |
| | loop-symmetric_ | |
| | UAGAAG-GACAGC | |
| 180 | -12_6-6_internal_ | GAUAUUGUGACAUAUCUCUGCACUCAU |
| | loop-symmetric_ | CACACAAAAGAUGCAAAAGAGGCAUA |
| | GAUUAG-AACCCG | GGAUCCACAGGGAGGGGGCAUUUUAA |
| | 0_1-1_mismatch_ | CAUAUCUCUGAAAACCCGAUC |
| | A-C | |
| | 30_6-6_internal_ | |
| | loop-symmetric_ | |
| | AGAAGA-AGAGGC | |
| 181 | -12_6-6_internal_ | GAUAUUGUGACAUAUCUCUGCACUCAU |
| | loop-symmetric_ | CACACAAAAGAUGCAAAGUAUGGAUA |
| | GAUUAG-GCCUGG | GGAUCCACAGGGAGGGGGCAUUUCAA |
| | 3_1-1_mismatch_ | UAUAUCUCUGAAGCCUGGAUC |
| | A-C | |
| | 30_6-6_internal_ | |
| | loop-symmetric_ | |
| | AGAAGA-GUAUGG | |
| 182 | -12_6-6_internal_ | GAUAUUGUGACAUAUCUCUGCACUCAU |
| | loop-symmetric_ | CACACAAAAGAUGCAAAAGAGCGAUA |
| | GAUUAG-AUUCGG | GGAUCCACAGGGAGGGGGCAUUCUAA |
| | 4_1-1_mismatch_ | UAUAUCUCUGAAAUUCGGAUC |
| | A-C | |
| | 30_6-6_internal_ | |
| | loop-symmetric_ | |
| | AGAAGA-AGAGCG | |
| 183 | -12_6-6_internal_ | GAUAUUGUGACAUAUCUCUGCACUCAU |
| | loop-symmetric_ | CACACAAAAGAUGCAAAAGUAAGAUA |
| | GAUUAG-AUUCGG | GGAUCCACAGGGAGGGGGCAUCUUAA |
| | 5_1-1_mismatch_ | UAUAUCUCUGAAAUUCGGAUC |
| | A-C | |
| | 30_6-6_internal_ | |
| | loop-symmetric_ | |
| | AGAAGA-AGUAAG | |
| 184 | -12_6-6_internal_ | GAUAUUGUGACAUAUCUCUGCACUCAU |
| | loop-symmetric_ | CACACAAAAGAUGCAAAAACCAAAUAG |
| | GAUUAG-AGGGCG | GAUCCACAGGGAGGGGGCAUUUUAAU |
| | 30_6-6_internal_ | AUAUCUCUGAAAGGGCGAUC |
| | loop-symmetric_ | |
| | AGAAGA-AACCAA | |
| 185 | -12_6-6_internal_ | GAUAUUGUGACAUAUCUCUGCACUCAU |
| | loop-symmetric_ | CACACAAAAGAUGCAAUAAAUAUAUA |
| | GAUUAG-GGUUGG | GGAUCCACAGGGAGGGGGCAUUUUAA |
| | 0_1-1_mismatch_ | CAUAUCUCUGAAGGUUGGAUC |
| | A-C | |
| | 31_6-6_internal_ | |
| | loop-symmetric_ | |
| | GAAGAU-UAAAUA | |
| 186 | -12_6-6_internal_ | GAUAUUGUGACAUAUCUCUGCACUCAU |
| | loop-symmetric_ | CACACAAAAGAUGCAACAUGAAUAUA |
| | GAUUAG-GGCGAG | GGAUCCACAGGGAGGGGGCAUCUUAA |
| | 5_1-1_mismatch_ | UAUAUCUCUGAAGGCGAGAUC |
| | A-C | |
| | 31_6-6_internal_ | |
| | loop-symmetric_ | |
| | GAAGAU-CAUGAA | |
| 187 | -12_6-6_internal_ | GAUAUUGUGACAUAUCUCUGCACUCAU |
| | loop-symmetric_ | CACACAAAAGAUGCAUCAAGACUAUAG |
| | GAUUAG-GCCGCG | GAUCCACAGGGAGGGGGCAUUUUAAC |
| | 0_1-1_mismatch_ | AUAUCUCUGAAGCCGCGAUC |
| | A-C | |
| | 32_6-6_internal_ | |
| | loop-symmetric_ | |
| | AAGAUU-UCAAGA | |
| 188 | -12_6-6_internal_ | GAUAUUGUGACAUAUCUCUGCACUCAU |
| | loop-symmetric_ | CACACAAAAGAUGCACAGGACCUAUAG |
| | GAUUAG-GCCUAA | GAUCCACAGGGAGGGGGCAUUUCAAU |
| | 3_1-1_mismatch_ | AUAUCUCUGAAGCCUAAAUC |
| | A-C | |
| | 32_6-6_internal_ | |
| | loop-symmetric_ | |
| | AAGAUU-CAGGAC | |
| 189 | -12_6-6_internal_ | GAUAUUGUGACAUAUCUCUGCACUCAU |
| | loop-symmetric_ | CACACAAAAGAUGCACCUAGACUAUAG |
| | GAUUAG-AAUACA | GAUCCACAGGGAGGGGGCAUUCUAAU |
| | 4_1-1_mismatch_ | AUAUCUCUGAAAAUACAAUC |
| | A-C | |
| | 32_6-6_internal_ | |
| | loop-symmetric_ | |
| | AAGAUU-CCUAGA | |
| 190 | -12_6-6_internal_ | GAUAUUGUGACAUAUCUCUGCACUCAU |
| | loop-symmetric_ | CACACAAAAGAUGCACAAUAACUAUAG |
| | GAUUAG-ACCCGA | GAUCCACAGGGAGGGGGCAUCUUAAU |
| | 5_1-1_mismatch_ | AUAUCUCUGAAACCCGAAUC |
| | A-C | |
| | 32_6-6_internal_ | |
| | loop-symmetric_ | |
| | AAGAUU-CAAUAA | |
| 191 | -12_6-6_internal_ | GAUAUUGUGACAUAUCUCUGCACUCAU |
| | loop-symmetric_ | CACACAAAAGAUGCACUGAUACUAUAG |
| | GAUUAG-GCAUAG | GAUCCACAGGGAGGGGGCAUUUUAAU |
| | 32_6-6_internal_ | AUAUCUCUGAAGCAUAGAUC |
| | loop-symmetric_ | |
| | AAGAUU-CUGAUA | |
| 192 | -12_6-6_internal_ | GAUAUUGUGACAUAUCUCUGCACUCAU |
| | loop-symmetric_ | CACACAAAAGAUGCUAACGCUCUAUAG |
| | GAUUAG-GCCGAA | GAUCCACAGGGAGGGGGCAUUUUAAC |
| | 0_1-1_mismatch_ | AUAUCUCUGAAGCCGAAAUC |
| | A-C | |
| | 33_6-6_internal_ | |
| | loop-symmetric_ | |
| | AGAUUU-UAACGC | |
| 193 | -12_6-6_internal_ | GAUAUUGUGACAUAUCUCUGCACUCAU |
| | loop-symmetric_ | CACACAAAAGAUGCUUUGGCUCUAUAG |
| | GAUUAG-GCGCGA | GAUCCACAGGGAGGGGGCAUUUCAAU |
| | 3_1-1_mismatch_ | AUAUCUCUGAAGCGCGAAUC |
| | A-C | |
| | 33_6-6_internal_ | |
| | loop-symmetric_ | |
| | AGAUUU-UUUGGC | |
| 194 | -12_6-6_internal_ | GAUAUUGUGACAUAUCUCUGCACUCAU |
| | loop-symmetric_ | CACACAAAAGAUGCCCGCCCUCUAUAG |
| | GAUUAG-AGUAAA | GAUCCACAGGGAGGGGGCAUUCUAAU |
| | 4_1-1_mismatch_ | AUAUCUCUGAAAGUAAAAUC |
| | A-C | |
| | 33_6-6_internal_ | |
| | loop-symmetric_ | |
| | AGAUUU-CCGCCC | |
| 195 | -12_6-6_internal_ | GAUAUUGUGACAUAUCUCUGCACUCAU |
| | loop-symmetric_ | CACACAAAAGAUGCUCGGGAUCUAUAG |
| | GAUUAG-GCCCGG | GAUCCACAGGGAGGGGGCAUCUUAAU |
| | 5_1-1_mismatch_ | AUAUCUCUGAAGCCCGGAUC |
| | A-C | |
| | 33_6-6_internal_ | |
| | loop-symmetric_ | |
| | AGAUUU-UCGGGA | |
| 196 | -12_6-6_internal_ | GAUAUUGUGACAUAUCUCUGCACUCAU |
| | loop-symmetric_ | CACACAAAAGAUGCCCCUACUCUAUAG |
| | GAUUAG-GACAAA | GAUCCACAGGGAGGGGGCAUUUUAAU |
| | 33_6-6_internal_ | AUAUCUCUGAAGACAAAAUC |
| | loop-symmetric_ | |
| | AGAUUU-CCCUAC | |
| 197 | -12_6-6_internal_ | GAUAUUGUGACAUAUCUCUGCACUCAU |
| | loop-symmetric_ | CACACAAAAGAUGAGUCGAUUCUAUA |
| | GAUUAG-GCCACA | GGAUCCACAGGGAGGGGGCAUUUUAA |
| | 0_1-1_mismatch_ | CAUAUCUCUGAAGCCACAAUC |
| | A-C | |
| | 34_6-6_internal_ | |
| | loop-symmetric_ | |
| | GAUUUG-AGUCGA | |
| 198 | -12_6-6_internal_ | GAUAUUGUGACAUAUCUCUGCACUCAU |
| | loop-symmetric_ | CACACAAAAGAUGAGGGAGUUCUAUA |
| | GAUUAG-ACUCCA | GGAUCCACAGGGAGGGGGCAUUUCAA |
| | 3_1-1_mismatch_ | UAUAUCUCUGAAACUCCAAUC |
| | A-C | |
| | 34_6-6_internal_ | |
| | loop-symmetric_ | |
| | GAUUUG-AGGGAG | |
| 199 | -12_6-6_internal_ | GAUAUUGUGACAUAUCUCUGCACUCAU |
| | loop-symmetric_ | CACACAAAAGAUGAUACAAUUCUAUA |
| | GAUUAG-GCCGCA | GGAUCCACAGGGAGGGGGCAUUCUAA |
| | 4_1-1_mismatch_ | UAUAUCUCUGAAGCCGCAAUC |
| | A-C | |
| | 34_6-6_internal_ | |
| | loop-symmetric_ | |
| | GAUUUG-AUACAA | |
| 200 | -12_6-6_internal_ | GAUAUUGUGACAUAUCUCUGCACUCAU |
| | loop-symmetric_ | CACACAAAAGAUGGCUCCGUUCUAUAG |
| | GAUUAG-ACAAAA | GAUCCACAGGGAGGGGGCAUCUUAAU |
| | 5_1-1_mismatch_ | AUAUCUCUGAAACAAAAAUC |
| | A-C | |
| | 34_6-6_internal_ | |
| | loop-symmetric_ | |
| | GAUUUG-GCUCCG | |
| 201 | -12_6-6_internal_ | GAUAUUGUGACAUAUCUCUGCACUCAU |
| | loop-symmetric_ | CACACAAAAGAUGAAUGCGUUCUAUA |
| | GAUUAG-AGGUAA | GGAUCCACAGGGAGGGGGCAUUUUAA |
| | 34_6-6_internal_ | UAUAUCUCUGAAAGGUAAAUC |
| | loop-symmetric_ | |
| | GAUUUG-AAUGCG | |
| 202 | -12_6-6_internal_ | GAUAUUGUGACAUAUCUCUGCACUCAU |
| | loop-symmetric_ | CACACAAAAGAUCUGUCGCUUCUAUAG |
| | GAUUAG-AUGUAG | GAUCCACAGGGAGGGGGCAUUUUAAC |
| | 0_1-1_mismatch_ | AUAUCUCUGAAAUGUAGAUC |
| | A-C | |
| | 35_6-6_internal_ | |
| | loop-symmetric_ | |
| | AUUUGC-CUGUCG | |
| 203 | -12_6-6_internal_ | GAUAUUGUGACAUAUCUCUGCACUCAU |
| | loop-symmetric_ | CACACAAAAGAUUACCGACUUCUAUAG |
| | GAUUAG-AACCCA | GAUCCACAGGGAGGGGGCAUUUCAAU |
| | 3_1-1_mismatch_ | AUAUCUCUGAAAACCCAAUC |
| | A-C | |
| | 35_6-6_internal_ | |
| | loop-symmetric_ | |
| | AUUUGC-UACCGA | |
| 204 | -12_6-6_internal_ | GAUAUUGUGACAUAUCUCUGCACUCAU |
| | loop-symmetric_ | CACACAAAAGAUAACUAGCUUCUAUAG |
| | GAUUAG-AGUGCA | GAUCCACAGGGAGGGGGCAUUCUAAU |
| | 4_1-1_mismatch_ | AUAUCUCUGAAAGUGCAAUC |
| | A-C | |
| | 35_6-6_internal_ | |
| | loop-symmetric_ | |
| | AUUUGC-AACUAG | |
| 205 | -12_6-6_internal_ | GAUAUUGUGACAUAUCUCUGCACUCAU |
| | loop-symmetric_ | CACACAAAAGAUAACCGACUUCUAUAG |
| | GAUUAG-GGGAGG | GAUCCACAGGGAGGGGGCAUCUUAAU |
| | 5_1-1_mismatch_ | AUAUCUCUGAAGGGAGGAUC |
| | A-C | |
| | 35_6-6_internal_ | |
| | loop-symmetric_ | |
| | AUUUGC-AACCGA | |
| 206 | -12_6-6_internal_ | GAUAUUGUGACAUAUCUCUGCACUCAU |
| | loop-symmetric_ | CACACAAAAGAUUACGCCCUUCUAUAG |
| | GAUUAG-AGCGAG | GAUCCACAGGGAGGGGGCAUUUUAAU |
| | 35_6-6_internal_ | AUAUCUCUGAAAGCGAGAUC |
| | loop-symmetric_ | |
| | AUUUGC-UACGCC | |
| 207 | -12_6-6_internal_ | GAUAUUGUGACAUAUCUCUGCACUCAU |
| | loop-symmetric_ | CACACAAAAGAAUUUCCUCUUCUAUAG |
| | GAUUAG-GGCGAA | GAUCCACAGGGAGGGGGCAUUUUAAC |
| | 0_1-1_mismatch_ | AUAUCUCUGAAGGCGAAAUC |
| | A-C | |
| | 36_6-6_internal_ | |
| | loop-symmetric_ | |
| | UUUGCA-AUUUCC | |
| 208 | -12_6-6_internal_ | GAUAUUGUGACAUAUCUCUGCACUCAU |
| | loop-symmetric_ | CACACAAAAGAACUACUUCUUCUAUAG |
| | GAUUAG-AUUUAA | GAUCCACAGGGAGGGGGCAUUUCAAU |
| | 3_1-1_mismatch_ | AUAUCUCUGAAAUUUAAAUC |
| | A-C | |
| | 36_6-6_internal_ | |
| | loop-symmetric_ | |
| | UUUGCA-ACUACU | |
| 209 | -12_6-6_internal_ | GAUAUUGUGACAUAUCUCUGCACUCAU |
| | loop-symmetric_ | CACACAAAAGAGGAGCUUCUUCUAUAG |
| | GAUUAG-AAGGGG | GAUCCACAGGGAGGGGGCAUUCUAAU |
| | 4_1-1_mismatch_ | AUAUCUCUGAAAAGGGGAUC |
| | A-C | |
| | 36_6-6_internal_ | |
| | loop-symmetric_ | |
| | UUUGCA-GGAGCU | |
| 210 | -12_6-6_internal_ | GAUAUUGUGACAUAUCUCUGCACUCAU |
| | loop-symmetric_ | CACACAAAAGACCAUAUUCUUCUAUAG |
| | GAUUAG-AAUAGG | GAUCCACAGGGAGGGGGCAUCUUAAU |
| | 5_1-1_mismatch_ | AUAUCUCUGAAAAUAGGAUC |
| | A-C | |
| | 36_6-6_internal_ | |
| | loop-symmetric_ | |
| | UUUGCA-CCAUAU | |
| 211 | -12_6-6_internal_ | GAUAUUGUGACAUAUCUCUGCACUCAU |
| | loop-symmetric_ | CACACAAAAGAAUUCCUUCUUCUAUAG |
| | GAUUAG-GAUUAG | GAUCCACAGGGAGGGGGCAUUUUAAU |
| | 36_6-6_internal_ | AUAUCUCUGAAGAUUAGAUC |
| | loop-symmetric_ | |
| | UUUGCA-AUUCCU | |
| 212 | -12_6-6_internal_ | GAUAUUGUGACAUAUCUCUGCACUCAU |
| | loop-symmetric_ | CACACAAAAGCUUAAUAUCUUCUAUAG |
| | GAUUAG-AAUUGG | GAUCCACAGGGAGGGGGCAUUUUAAC |
| | 0_1-1_mismatch_ | AUAUCUCUGAAAAUUGGAUC |
| | A-C | |
| | 37_6-6_internal_ | |
| | loop-symmetric_ | |
| | UUGCAU-CUUAAU | |
| 213 | -12_6-6_internal_ | GAUAUUGUGACAUAUCUCUGCACUCAU |
| | loop-symmetric_ | CACACAAAAGUCUAUUAUCUUCUAUAG |
| | GAUUAG-GGAGGG | GAUCCACAGGGAGGGGGCAUUUCAAU |
| | 3_1-1_mismatch_ | AUAUCUCUGAAGGAGGGAUC |
| | A-C | |
| | 37_6-6_internal_ | |
| | loop-symmetric_ | |
| | UUGCAU-UCUAUU | |
| 214 | -12_6-6_internal_ | GAUAUUGUGACAUAUCUCUGCACUCAU |
| | loop-symmetric_ | CACACAAAAGCAAUUUAUCUUCUAUAG |
| | GAUUAG-GCCCGA | GAUCCACAGGGAGGGGGCAUCUUAAU |
| | 5_1-1_mismatch_ | AUAUCUCUGAAGCCCGAAUC |
| | A-C | |
| | 37_6-6_internal_ | |
| | loop-symmetric_ | |
| | UUGCAU-CAAUUU | |
| 215 | -12_6-6_internal_ | GAUAUUGUGACAUAUCUCUGCACUCAU |
| | loop-symmetric_ | CACACAAAAGUACUCUAUCUUCUAUAG |
| | GAUUAG-GGUGCG | GAUCCACAGGGAGGGGGCAUUUUAAU |
| | 37_6-6_internal_ | AUAUCUCUGAAGGUGCGAUC |
| | loop-symmetric_ | |
| | UUGCAU-UACUCU | |
| 216 | -12_6-6_internal_ | GAUAUUGUGACAUAUCUCUGCACUCAU |
| | loop-symmetric_ | CACACAAAACUCAGUAAUCUUCUAUAG |
| | GAUUAG-GGUGGA | GAUCCACAGGGAGGGGGCAUUUUAAC |
| | 0_1-1_mismatch_ | AUAUCUCUGAAGGUGGAAUC |
| | A-C | |
| | 38_6-6_internal_ | |
| | loop-symmetric_ | |
| | UGCAUC-CUCAGU | |
| 217 | -12_6-6_internal_ | GAUAUUGUGACAUAUCUCUGCACUCAU |
| | loop-symmetric_ | CACACAAAAUUUUUUAAUCUUCUAUA |
| | GAUUAG-GGGCAG | GGAUCCACAGGGAGGGGGCAUUUCAA |
| | 3_1-1_mismatch_ | UAUAUCUCUGAAGGGCAGAUC |
| | A-C | |
| | 38_6-6_internal_ | |
| | loop-symmetric_ | |
| | UGCAUC-UUUUUU | |
| 218 | -12_6-6_internal_ | GAUAUUGUGACAUAUCUCUGCACUCAU |
| | loop-symmetric_ | CACACAAAAAACAGUAAUCUUCUAUAG |
| | GAUUAG-ACGGGA | GAUCCACAGGGAGGGGGCAUUCUAAU |
| | 4_1-1_mismatch_ | AUAUCUCUGAAACGGGAAUC |
| | A-C | |
| | 38_6-6_internal_ | |
| | loop-symmetric_ | |
| | UGCAUC-AACAGU | |
| 219 | -12_6-6_internal_ | GAUAUUGUGACAUAUCUCUGCACUCAU |
| | loop-symmetric_ | CACACAAAAACGAACAAUCUUCUAUAG |
| | GAUUAG-GGACGG | GAUCCACAGGGAGGGGGCAUCUUAAU |
| | 5_1-1_mismatch_ | AUAUCUCUGAAGGACGGAUC |
| | A-C | |
| | 38_6-6_internal_ | |
| | loop-symmetric_ | |
| | UGCAUC-ACGAAC | |
| 220 | -12_6-6_internal_ | GAUAUUGUGACAUAUCUCUGCACUCAU |
| | loop-symmetric_ | CACACAAAAAUCUUUAAUCUUCUAUAG |
| | GAUUAG-GAUGCG | GAUCCACAGGGAGGGGGCAUUUUAAU |
| | 38_6-6_internal_ | AUAUCUCUGAAGAUGCGAUC |
| | loop-symmetric_ | |
| | UGCAUC-AUCUUU | |
| 221 | -12_6-6_internal_ | GAUAUUGUGACAUAUCUCUGCACUCAU |
| | loop-symmetric_ | CACACAAACUCUAGAAAUCUUCUAUAG |
| | GAUUAG-GCGGAG | GAUCCACAGGGAGGGGGCAUUUUAAC |
| | 0_1-1_mismatch_ | AUAUCUCUGAAGCGGAGAUC |
| | A-C | |
| | 39_6-6_internal_ | |
| | loop-symmetric_ | |
| | GCAUCU-CUCUAG | |
| 222 | -12_6-6_internal_ | GAUAUUGUGACAUAUCUCUGCACUCAU |
| | loop-symmetric_ | CACACAAAUUGACAAAAUCUUCUAUAG |
| | GAUUAG-AUGCGG | GAUCCACAGGGAGGGGGCAUUUCAAU |
| | 3_1-1_mismatch_ | AUAUCUCUGAAAUGCGGAUC |
| | A-C | |
| | 39_6-6_internal_ | |
| | loop-symmetric_ | |
| | GCAUCU-UUGACA | |
| 223 | -12_6-6_internal_ | GAUAUUGUGACAUAUCUCUGCACUCAU |
| | loop-symmetric_ | CACACAAAUUCGUAAAAUCUUCUAUAG |
| | GAUUAG-GACAAA | GAUCCACAGGGAGGGGGCAUCUUAAU |
| | 5_1-1_mismatch_ | AUAUCUCUGAAGACAAAAUC |
| | A-C | |
| | 39_6-6_internal_ | |
| | loop-symmetric_ | |
| | GCAUCU-UUCGUA | |
| 224 | -12_6-6_internal_ | GAUAUUGUGACAUAUCUCUGCACUCAU |
| | loop-symmetric_ | CACACAACAUUAACAAAUCUUCUAUAG |
| | GAUUAG-AUCCGA | GAUCCACAGGGAGGGGGCAUUUUAAC |
| | 0_1-1_mismatch_ | AUAUCUCUGAAAUCCGAAUC |
| | A-C | |
| | 40_6-6_internal_ | |
| | loop-symmetric_ | |
| | CAUCUU-CAUUAA | |
| 225 | -12_6-6_internal_ | GAUAUUGUGACAUAUCUCUGCACUCAU |
| | loop-symmetric_ | CACACAAUCCCCCCAAAUCUUCUAUAG |
| | GAUUAG-GCUCAA | GAUCCACAGGGAGGGGGCAUUUCAAU |
| | 3_1-1_mismatch_ | AUAUCUCUGAAGCUCAAAUC |
| | A-C | |
| | 40_6-6_internal_ | |
| | loop-symmetric_ | |
| | CAUCUU-UCCCCC | |
| 226 | -12_6-6_internal_ | GAUAUUGUGACAUAUCUCUGCACUCAU |
| | loop-symmetric_ | CACACAAUACGCCCAAAUCUUCUAUAG |
| | GAUUAG-AGCAAA | GAUCCACAGGGAGGGGGCAUUCUAAU |
| | 4_1-1_mismatch_ | AUAUCUCUGAAAGCAAAAUC |
| | A-C | |
| | 40_6-6_internal_ | |
| | loop-symmetric_ | |
| | CAUCUU-UACGCC | |
| 227 | -12_6-6_internal_ | GAUAUUGUGACAUAUCUCUGCACUCAU |
| | loop-symmetric_ | CACACAACUACAUCAAAUCUUCUAUAG |
| | GAUUAG-AAGUGA | GAUCCACAGGGAGGGGGCAUCUUAAU |
| | 5_1-1_mismatch_ | AUAUCUCUGAAAAGUGAAUC |
| | A-C | |
| | 40_6-6_internal_ | |
| | loop-symmetric_ | |
| | CAUCUU-CUACAU | |
| 228 | -12_6-6_internal_ | GAUAUUGUGACAUAUCUCUGCACUCAU |
| | loop-symmetric_ | CACACAACCGCGCCAAAUCUUCUAUAG |
| | GAUUAG-GCCAUA | GAUCCACAGGGAGGGGGCAUUUUAAU |
| | 40_6-6_internal_ | AUAUCUCUGAAGCCAUAAUC |
| | loop-symmetric_ | |
| | CAUCUU-CCGCGC | |
| 229 | -12_6-6_internal_ | GAUAUUGUGACAUAUCUCUGCACUCAU |
| | loop-symmetric_ | CACACACCCGCGGCAAAUCUUCUAUAG |
| | GAUUAG-GCACGG | GAUCCACAGGGAGGGGGCAUUUUAAC |
| | 0_1-1_mismatch_ | AUAUCUCUGAAGCACGGAUC |
| | A-C | |
| | 41_6-6_internal_ | |
| | loop-symmetric_ | |
| | AUCUUU-CCCGCG | |
| 230 | -12_6-6_internal_ | GAUAUUGUGACAUAUCUCUGCACUCAU |
| | loop-symmetric_ | CACACACGCUUAGCAAAUCUUCUAUAG |
| | GAUUAG-ACGUAG | GAUCCACAGGGAGGGGGCAUUUCAAU |
| | 3_1-1_mismatch_ | AUAUCUCUGAAACGUAGAUC |
| | A-C | |
| | 41_6-6_internal_ | |
| | loop-symmetric_ | |
| | AUCUUU-CGCUUA | |
| 231 | -12_6-6_internal_ | GAUAUUGUGACAUAUCUCUGCACUCAU |
| | loop-symmetric_ | CACACAUCCCAAGCAAAUCUUCUAUAG |
| | GAUUAG-AGUUAG | GAUCCACAGGGAGGGGGCAUUCUAAU |
| | 4_1-1_mismatch_ | AUAUCUCUGAAAGUUAGAUC |
| | A-C | |
| | 41_6-6_internal_ | |
| | loop-symmetric_ | |
| | AUCUUU-UCCCAA | |
| 232 | -12_6-6_internal_ | GAUAUUGUGACAUAUCUCUGCACUCAU |
| | loop-symmetric_ | CACACAUUCCAAGCAAAUCUUCUAUAG |
| | GAUUAG-ACACGG | GAUCCACAGGGAGGGGGCAUCUUAAU |
| | 5_1-1_mismatch_ | AUAUCUCUGAAACACGGAUC |
| | A-C | |
| | 41_6-6_internal_ | |
| | loop-symmetric_ | |
| | AUCUUU-UUCCAA | |
| 233 | -12_6-6_internal_ | GAUAUUGUGACAUAUCUCUGCACUCAU |
| | loop-symmetric_ | CACACAUCAUUAGCAAAUCUUCUAUAG |
| | GAUUAG-AUUGGG | GAUCCACAGGGAGGGGGCAUUUUAAU |
| | 41_6-6_internal_ | AUAUCUCUGAAAUUGGGAUC |
| | loop-symmetric_ | |
| | AUCUUU-UCAUUA | |
| 234 | -12_6-6_internal_ | GAUAUUGUGACAUAUCUCUGCACUCAU |
| | loop-symmetric_ | CACACCGUAUCUGCAAAUCUUCUAUAG |
| | GAUUAG-GGGACG | GAUCCACAGGGAGGGGGCAUUUUAAC |
| | 0_1-1_mismatch_ | AUAUCUCUGAAGGGACGAUC |
| | A-C | |
| | 42_6-6_internal_ | |
| | loop-symmetric_ | |
| | UCUUUU-CGUAUC | |
| 235 | -12_6-6_internal_ | GAUAUUGUGACAUAUCUCUGCACUCAU |
| | loop-symmetric_ | CACACCUGGCCUGCAAAUCUUCUAUAG |
| | GAUUAG-AGGCGA | GAUCCACAGGGAGGGGGCAUUUCAAU |
| | 3_1-1_mismatch_ | AUAUCUCUGAAAGGCGAAUC |
| | A-C | |
| | 42_6-6_internal_ | |
| | loop-symmetric_ | |
| | UCUUUU-CUGGCC | |
| 236 | -12_6-6_internal_ | GAUAUUGUGACAUAUCUCUGCACUCAU |
| | loop-symmetric_ | CACACUAACGUUGCAAAUCUUCUAUAG |
| | GAUUAG-ACGCAG | GAUCCACAGGGAGGGGGCAUUCUAAU |
| | 4_1-1_mismatch_ | AUAUCUCUGAAACGCAGAUC |
| | A-C | |
| | 42_6-6_internal_ | |
| | loop-symmetric_ | |
| | UCUUUU-UAACGU | |
| 237 | -12_6-6_internal_ | GAUAUUGUGACAUAUCUCUGCACUCAU |
| | loop-symmetric_ | CACACUGUGCUUGCAAAUCUUCUAUAG |
| | GAUUAG-GCCAGA | GAUCCACAGGGAGGGGGCAUUUUAAU |
| | 42_6-6_internal_ | AUAUCUCUGAAGCCAGAAUC |
| | loop-symmetric_ | |
| | UCUUUU-UGUGCU | |
| 238 | -12_6-6_internal_ | GAUAUUGUGACAUAUCUCUGCACUCAU |
| | loop-symmetric_ | CACAGCCACAAUGCAAAUCUUCUAUAG |
| | GAUUAG-GCCACA | GAUCCACAGGGAGGGGGCAUUUUAAC |
| | 0_1-1_mismatch_ | AUAUCUCUGAAGCCACAAUC |
| | A-C | |
| | 43_6-6_internal_ | |
| | loop-symmetric_ | |
| | CUUUUG-GCCACA | |
| 239 | -12_6-6_internal_ | GAUAUUGUGACAUAUCUCUGCACUCAU |
| | loop-symmetric_ | CACAACUCUCAUGCAAAUCUUCUAUAG |
| | GAUUAG-AAUUGA | GAUCCACAGGGAGGGGGCAUUCUAAU |
| | 4_1-1_mismatch_ | AUAUCUCUGAAAAUUGAAUC |
| | A-C | |
| | 43_6-6_internal_ | |
| | loop-symmetric_ | |
| | CUUUUG-ACUCUC | |
| 240 | -12_6-6_internal_ | GAUAUUGUGACAUAUCUCUGCACUCAU |
| | loop-symmetric_ | CACAAUGUACAUGCAAAUCUUCUAUAG |
| | GAUUAG-GGACAG | GAUCCACAGGGAGGGGGCAUCUUAAU |
| | 5_1-1_mismatch_ | AUAUCUCUGAAGGACAGAUC |
| | A-C | |
| | 43_6-6_internal_ | |
| | loop-symmetric_ | |
| | CUUUUG-AUGUAC | |
| 241 | -12_6-6_internal_ | GAUAUUGUGACAUAUCUCUGCACUCAU |
| | loop-symmetric_ | CACAAGCGCUAUGCAAAUCUUCUAUAG |
| | GAUUAG-AAACAA | GAUCCACAGGGAGGGGGCAUUUUAAU |
| | 43_6-6_internal_ | AUAUCUCUGAAAAACAAAUC |
| | loop-symmetric_ | |
| | CUUUUG-AGCGCU | |
| 242 | -12_6-6_internal_ | GAUAUUGUGACAUAUCUCUGCACUCAU |
| | loop-symmetric_ | CACUAUGUUGAUGCAAAUCUUCUAUA |
| | GAUUAG-GCCUAG | GGAUCCACAGGGAGGGGGCAUUUUAA |
| | 0_1-1_mismatch_ | CAUAUCUCUGAAGCCUAGAUC |
| | A-C | |
| | 44_6-6_internal_ | |
| | loop-symmetric_ | |
| | UUUUGU-UAUGUU | |
| 243 | -12_6-6_internal_ | GAUAUUGUGACAUAUCUCUGCACUCAU |
| | loop-symmetric_ | CACUAAUCCGAUGCAAAUCUUCUAUAG |
| | GAUUAG-GGACGA | GAUCCACAGGGAGGGGGCAUUUCAAU |
| | 3_1-1_mismatch_ | AUAUCUCUGAAGGACGAAUC |
| | A-C | |
| | 44_6-6_internal_ | |
| | loop-symmetric_ | |
| | UUUUGU-UAAUCC | |
| 244 | -12_6-6_internal_ | GAUAUUGUGACAUAUCUCUGCACUCAU |
| | loop-symmetric_ | CACUUUAGUGAUGCAAAUCUUCUAUA |
| | GAUUAG-AUCCAA | GGAUCCACAGGGAGGGGGCAUUCUAA |
| | 4_1-1_mismatch_ | UAUAUCUCUGAAAUCCAAAUC |
| | A-C | |
| | 44_6-6_internal_ | |
| | loop-symmetric_ | |
| | UUUUGU-UUUAGU | |
| 245 | -12_6-6_internal_ | GAUAUUGUGACAUAUCUCUGCACUCAU |
| | loop-symmetric_ | CACUAUAUCGAUGCAAAUCUUCUAUAG |
| | GAUUAG-GCCCGA | GAUCCACAGGGAGGGGGCAUCUUAAU |
| | 5_1-1_mismatch_ | AUAUCUCUGAAGCCCGAAUC |
| | A-C | |
| | 44_6-6_internal_ | |
| | loop-symmetric_ | |
| | UUUUGU-UAUAUC | |
| 246 | -12_6-6_internal_ | GAUAUUGUGACAUAUCUCUGCACUCAU |
| | loop-symmetric_ | CACCUGUACGAUGCAAAUCUUCUAUAG |
| | GAUUAG-GAAGAG | GAUCCACAGGGAGGGGGCAUUUUAAU |
| | 44_6-6_internal_ | AUAUCUCUGAAGAAGAGAUC |
| | loop-symmetric_ | |
| | UUUUGU-CUGUAC | |
| 247 | -11_6-6_internal_ | GAUAUUGUGACAUAUCUCUGCACUCAU |
| | loop-symmetric_ | CACACAAAAGAUGCAAAUCUUCUAUAG |
| | AUUAGU-UAACCG | GAUCCACAGGGAGGGGGCAUUUUAAC |
| | 0_1-1_mismatch_ | AUAUCUCUGAUAACCGCAUC |
| | A-C | |
| 248 | -11_6-6_internal_ | GAUAUUGUGACAUAUCUCUGCACUCAU |
| | loop-symmetric_ | CACACAAAAGAUGCAAAUCUUCUAUAG |
| | AUUAGU-CCAGGG | GAUCCACAGGGAGGGGGCAUUUCAAU |
| | 3_1-1_mismatch_ | AUAUCUCUGACCAGGGCAUC |
| | A-C | |
| 249 | -11_6-6_internal_ | GAUAUUGUGACAUAUCUCUGCACUCAU |
| | loop-symmetric_ | CACACAAAAGAUGCAAAUCUUCUAUAG |
| | AUUAGU-UUGUAG | GAUCCACAGGGAGGGGGCAUUCUAAU |
| | 4_1-1_mismatch_ | AUAUCUCUGAUUGUAGCAUC |
| | A-C | |
| 250 | -11_6-6_internal_ | GAUAUUGUGACAUAUCUCUGCACUCAU |
| | loop-symmetric_ | CACACAAAAGAUGCAAAUCUUCUAUAG |
| | AUUAGU-CAAUAC | GAUCCACAGGGAGGGGGCAUCUUAAU |
| | 5_1-1_mismatch_ | AUAUCUCUGACAAUACCAUC |
| | A-C | |
| 251 | -11_6-6_internal_ | GAUAUUGUGACAUAUCUCUGCACUCAU |
| | loop-symmetric_ | CACACAAAAGAUGCAAAUCUUCUAUAG |
| | AUUAGU-UUCGUG | GAUCCACAGGGAGGGGGCAUUUUAAU |
| | | AUAUCUCUGAUUCGUGCAUC |
| 252 | -11_6-6_internal_ | GAUAUUGUGACAUAUCUCUGCACUCAU |
| | loop-symmetric_ | CACACAAAAGAUGCAAAUCUUCUAUAG |
| | AUUAGU-UAAUCC | AGCGGUCAGGGAGGGGGCAUUUUAAC |
| | 0_1-1_mismatch_ | AUAUCUCUGAUAAUCCCAUC |
| | A-C | |
| | 20_6-6_internal_ | |
| | loop-symmetric_ | |
| | UGGAUC-AGCGGU | |
| 253 | -11_6-6_internal_ | GAUAUUGUGACAUAUCUCUGCACUCAU |
| | loop-symmetric_ | CACACAAAAGAUGCAAAUCUUCUAUAG |
| | AUUAGU-UAAUUG | UCAGGUCAGGGAGGGGGCAUUUCAAU |
| | 3_1-1_mismatch_ | AUAUCUCUGAUAAUUGCAUC |
| | A-C | |
| | 20_6-6_internal_ | |
| | loop-symmetric_ | |
| | UGGAUC-UCAGGU | |
| 254 | -11_6-6_internal_ | GAUAUUGUGACAUAUCUCUGCACUCAU |
| | loop-symmetric_ | CACACAAAAGAUGCAAAUCUUCUAUAG |
| | AUUAGU-UCCUCG | ACGGGUCAGGGAGGGGGCAUUCUAAU |
| | 4_1-1_mismatch_ | AUAUCUCUGAUCCUCGCAUC |
| | A-C | |
| | 20_6-6_internal_ | |
| | loop-symmetric_ | |
| | UGGAUC-ACGGGU | |
| 255 | -11_6-6_internal_ | GAUAUUGUGACAUAUCUCUGCACUCAU |
| | loop-symmetric_ | CACACAAAAGAUGCAAAUCUUCUAUAG |
| | AUUAGU-CGGGGA | ACAAAUCAGGGAGGGGGCAUCUUAAU |
| | 5_1-1_mismatch_ | AUAUCUCUGACGGGGACAUC |
| | A-C | |
| | 20_6-6_internal_ | |
| | loop-symmetric_ | |
| | UGGAUC-ACAAAU | |
| 256 | -11_6-6_internal_ | GAUAUUGUGACAUAUCUCUGCACUCAU |
| | loop-symmetric_ | CACACAAAAGAUGCAAAUCUUCUAUAG |
| | AUUAGU-CAGCAA | ACGGGUCAGGGAGGGGGCAUUUUAAU |
| | 20_6-6_internal_ | AUAUCUCUGACAGCAACAUC |
| | loop-symmetric_ | |
| | UGGAUC-ACGGGU | |
| 257 | -11_6-6_internal_ | GAUAUUGUGACAUAUCUCUGCACUCAU |
| | loop-symmetric_ | CACACAAAAGAUGCAAAUCUUCUAUAC |
| | AUUAGU-CAGUUA | ACUAGACAGGGAGGGGGCAUUUUAAC |
| | 0_1-1_mismatch_ | AUAUCUCUGACAGUUACAUC |
| | A-C | |
| | 21_6-6_internal_ | |
| | loop-symmetric_ | |
| | GGAUCC-CACUAG | |
| 258 | -11_6-6_internal_ | GAUAUUGUGACAUAUCUCUGCACUCAU |
| | loop-symmetric_ | CACACAAAAGAUGCAAAUCUUCUAUAA |
| | AUUAGU-CAGAAG | UUACAACAGGGAGGGGGCAUUUCAAU |
| | 3_1-1_mismatch_ | AUAUCUCUGACAGAAGCAUC |
| | A-C | |
| | 21_6-6_internal_ | |
| | loop-symmetric_ | |
| | GGAUCC-AUUACA | |
| 259 | -11_6-6_internal_ | GAUAUUGUGACAUAUCUCUGCACUCAU |
| | loop-symmetric_ | CACACAAAAGAUGCAAAUCUUCUAUAC |
| | AUUAGU-CGGUUC | CUUAGACAGGGAGGGGGCAUUCUAAU |
| | 4_1-1_mismatch_ | AUAUCUCUGACGGUUCCAUC |
| | A-C | |
| | 21_6-6_internal_ | |
| | loop-symmetric_ | |
| | GGAUCC-CCUUAG | |
| 260 | -11_6-6_internal_ | GAUAUUGUGACAUAUCUCUGCACUCAU |
| | loop-symmetric_ | CACACAAAAGAUGCAAAUCUUCUAUAU |
| | AUUAGU-CCCCAC | AUGAGACAGGGAGGGGGCAUCUUAAU |
| | 5_1-1_mismatch_ | AUAUCUCUGACCCCACCAUC |
| | A-C | |
| | 21_6-6_internal_ | |
| | loop-symmetric_ | |
| | GGAUCC-UAUGAG | |
| 261 | -11_6-6_internal_ | GAUAUUGUGACAUAUCUCUGCACUCAU |
| | loop-symmetric_ | CACACAAAAGAUGCAAAUCUUCUAUAA |
| | AUUAGU-UCCUUG | CCAAGACAGGGAGGGGGCAUUUUAAU |
| | 21_6-6_internal_ | AUAUCUCUGAUCCUUGCAUC |
| | loop-symmetric_ | |
| | GGAUCC-ACCAAG | |
| 262 | -11_6-6_internal_ | GAUAUUGUGACAUAUCUCUGCACUCAU |
| | loop-symmetric_ | CACACAAAAGAUGCAAAUCUUCUAUUU |
| | AUUAGU-UGACAA | UCGGCACAGGGAGGGGGCAUUUUAAC |
| | 0_1-1_mismatch_ | AUAUCUCUGAUGACAACAUC |
| | A-C | |
| | 22_6-6_internal_ | |
| | loop-symmetric_ | |
| | GAUCCU-UUUCGG | |
| 263 | -11_6-6_internal_ | GAUAUUGUGACAUAUCUCUGCACUCAU |
| | loop-symmetric_ | CACACAAAAGAUGCAAAUCUUCUAUUU |
| | AUUAGU-CAUUUG | AGAACACAGGGAGGGGGCAUUUCAAU |
| | 3_1-1_mismatch_ | AUAUCUCUGACAUUUGCAUC |
| | A-C | |
| | 22_6-6_internal_ | |
| | loop-symmetric_ | |
| | GAUCCU-UUAGAA | |
| 264 | -11_6-6_internal_ | GAUAUUGUGACAUAUCUCUGCACUCAU |
| | loop-symmetric_ | CACACAAAAGAUGCAAAUCUUCUAUCA |
| | AUUAGU-CAGGUG | CCGGCACAGGGAGGGGGCAUUCUAAUA |
| | 4_1-1_mismatch_ | UAUCUCUGACAGGUGCAUC |
| | A-C | |
| | 22_6-6_internal_ | |
| | loop-symmetric_ | |
| | GAUCCU-CACCGG | |
| 265 | -11_6-6_internal_ | GAUAUUGUGACAUAUCUCUGCACUCAU |
| | loop-symmetric_ | CACACAAAAGAUGCAAAUCUUCUAUUU |
| | AUUAGU-CAUCUG | ACAGCACAGGGAGGGGGCAUCUUAAU |
| | 5_1-1_mismatch_ | AUAUCUCUGACAUCUGCAUC |
| | A-C | |
| | 22_6-6_internal_ | |
| | loop-symmetric_ | |
| | GAUCCU-UUACAG | |
| 266 | -11_6-6_internal_ | GAUAUUGUGACAUAUCUCUGCACUCAU |
| | loop-symmetric_ | CACACAAAAGAUGCAAAUCUUCUAUUU |
| | AUUAGU-UCCUUC | AUGACACAGGGAGGGGGCAUUUUAAU |
| | 22_6-6_internal_ | AUAUCUCUGAUCCUUCCAUC |
| | loop-symmetric_ | |
| | GAUCCU-UUAUGA | |
| 267 | -11_6-6_internal_ | GAUAUUGUGACAUAUCUCUGCACUCAU |
| | loop-symmetric_ | CACACAAAAGAUGCAAAUCUUCUACUC |
| | AUUAGU-UUCUCA | GCACCACAGGGAGGGGGCAUUUUAACA |
| | 0_1-1_mismatch_ | UAUCUCUGAUUCUCACAUC |
| | A-C | |
| | 23_6-6_internal_ | |
| | loop-symmetric_ | |
| | AUCCUA-CUCGCA | |
| 268 | -11_6-6_internal_ | GAUAUUGUGACAUAUCUCUGCACUCAU |
| | loop-symmetric_ | CACACAAAAGAUGCAAAUCUUCUACGA |
| | AUUAGU-CGGUGC | AACCCACAGGGAGGGGGCAUUUCAAUA |
| | 3_1-1_mismatch_ | UAUCUCUGACGGUGCCAUC |
| | A-C | |
| | 23_6-6_internal_ | |
| | loop-symmetric_ | |
| | AUCCUA-CGAAAC | |
| 269 | -11_6-6_internal_ | GAUAUUGUGACAUAUCUCUGCACUCAU |
| | loop-symmetric_ | CACACAAAAGAUGCAAAUCUUCUACGC |
| | AUUAGU-UGGUGC | ACGCCACAGGGAGGGGGCAUUCUAAUA |
| | 4_1-1_mismatch_ | UAUCUCUGAUGGUGCCAUC |
| | A-C | |
| | 23_6-6_internal_ | |
| | loop-symmetric_ | |
| | AUCCUA-CGCACG | |
| 270 | -11_6-6_internal_ | GAUAUUGUGACAUAUCUCUGCACUCAU |
| | loop-symmetric_ | CACACAAAAGAUGCAAAUCUUCUAGUC |
| | AUUAGU-UACCAC | ACCCCACAGGGAGGGGGCAUCUUAAUA |
| | 5_1-1_mismatch_ | UAUCUCUGAUACCACCAUC |
| | A-C | |
| | 23_6-6_internal_ | |
| | loop-symmetric_ | |
| | AUCCUA-GUCACC | |
| 271 | -11_6-6_internal_ | GAUAUUGUGACAUAUCUCUGCACUCAU |
| | loop-symmetric_ | CACACAAAAGAUGCAAAUCUUCUAAUG |
| | AUUAGU-CAACGG | UCGCCACAGGGAGGGGGCAUUUUAAU |
| | 23_6-6_internal_ | AUAUCUCUGACAACGGCAUC |
| | loop-symmetric_ | |
| | AUCCUA-AUGUCG | |
| 272 | -11_6-6_internal_ | GAUAUUGUGACAUAUCUCUGCACUCAU |
| | loop-symmetric_ | CACACAAAAGAUGCAAAUCUUCUCAAU |
| | AUUAGU-UGAAGG | GCUCCACAGGGAGGGGGCAUUUUAACA |
| | 0_1-1_mismatch_ | UAUCUCUGAUGAAGGCAUC |
| | A-C | |
| | 24_6-6_internal_ | |
| | loop-symmetric_ | |
| | UCCUAU-CAAUGC | |
| 273 | -11_6-6_internal_ | GAUAUUGUGACAUAUCUCUGCACUCAU |
| | loop-symmetric_ | CACACAAAAGAUGCAAAUCUUCUCAUC |
| | AUUAGU-CAGUAC | AUUCCACAGGGAGGGGGCAUUUCAAU |
| | 3_1-1_mismatch_ | AUAUCUCUGACAGUACCAUC |
| | A-C | |
| | 24_6-6_internal_ | |
| | loop-symmetric_ | |
| | UCCUAU-CAUCAU | |
| 274 | -11_6-6_internal_ | GAUAUUGUGACAUAUCUCUGCACUCAU |
| | loop-symmetric_ | CACACAAAAGAUGCAAAUCUUCUCGAA |
| | AUUAGU-UGUUCG | GUUCCACAGGGAGGGGGCAUUCUAAU |
| | 4_1-1_mismatch_ | AUAUCUCUGAUGUUCGCAUC |
| | A-C | |
| | 24_6-6_internal_ | |
| | loop-symmetric_ | |
| | UCCUAU-CGAAGU | |
| 275 | -11_6-6_internal_ | GAUAUUGUGACAUAUCUCUGCACUCAU |
| | loop-symmetric_ | CACACAAAAGAUGCAAAUCUUCUCGCC |
| | AUUAGU-UAGCCA | AUUCCACAGGGAGGGGGCAUCUUAAU |
| | 5_1-1_mismatch_ | AUAUCUCUGAUAGCCACAUC |
| | A-C | |
| | 24_6-6_internal_ | |
| | loop-symmetric_ | |
| | UCCUAU-CGCCAU | |
| 276 | -11_6-6_internal_ | GAUAUUGUGACAUAUCUCUGCACUCAU |
| | loop-symmetric_ | CACACAAAAGAUGCAAAUCUUCUUGAA |
| | AUUAGU-CGACCG | UUUCCACAGGGAGGGGGCAUUUUAAU |
| | 24_6-6_internal_ | AUAUCUCUGACGACCGCAUC |
| | loop-symmetric_ | |
| | UCCUAU-UGAAUU | |
| 277 | -11_6-6_internal_ | GAUAUUGUGACAUAUCUCUGCACUCAU |
| | loop-symmetric_ | CACACAAAAGAUGCAAAUCUUCGCCGA |
| | AUUAGU-UAAAAA | AAUCCACAGGGAGGGGGCAUUUUAAC |
| | 0_1-1_mismatch_ | AUAUCUCUGAUAAAAACAUC |
| | A-C | |
| | 25_6-6_internal_ | |
| | loop-symmetric_ | |
| | CCUAUA-GCCGAA | |
| 278 | -11_6-6_internal_ | GAUAUUGUGACAUAUCUCUGCACUCAU |
| | loop-symmetric_ | CACACAAAAGAUGCAAAUCUUCGUGUC |
| | AUUAGU-CCAUGC | CAUCCACAGGGAGGGGGCAUUUCAAUA |
| | 3_1-1_mismatch_ | UAUCUCUGACCAUGCCAUC |
| | A-C | |
| | 25_6-6_internal_ | |
| | loop-symmetric_ | |
| | CCUAUA-GUGUCC | |
| 279 | -11_6-6_internal_ | GAUAUUGUGACAUAUCUCUGCACUCAU |
| | loop-symmetric_ | CACACAAAAGAUGCAAAUCUUCCCCGC |
| | AUUAGU-UCAACC | CAUCCACAGGGAGGGGGCAUCUUAAUA |
| | 5_1-1_mismatch_ | UAUCUCUGAUCAACCCAUC |
| | A-C | |
| | 25_6-6_internal_ | |
| | loop-symmetric_ | |
| | CCUAUA-CCCGCC | |
| 280 | -11_6-6_internal_ | GAUAUUGUGACAUAUCUCUGCACUCAU |
| | loop-symmetric_ | CACACAAAAGAUGCAAAUCUUCGUUCC |
| | AUUAGU-UAGUCC | CAUCCACAGGGAGGGGGCAUUUUAAU |
| | 25_6-6_internal_ | AUAUCUCUGAUAGUCCCAUC |
| | loop-symmetric_ | |
| | CCUAUA-GUUCCC | |
| 281 | -11_6-6_internal_ | GAUAUUGUGACAUAUCUCUGCACUCAU |
| | loop-symmetric_ | CACACAAAAGAUGCAAAUCUUAGAACA |
| | AUUAGU-UCCCGA | GAUCCACAGGGAGGGGGCAUUUUAAC |
| | 0_1-1_mismatch_ | AUAUCUCUGAUCCCGACAUC |
| | A-C | |
| | 26_6-6_internal_ | |
| | loop-symmetric_ | |
| | CUAUAG-AGAACA | |
| 282 | -11_6-6_internal_ | GAUAUUGUGACAUAUCUCUGCACUCAU |
| | loop-symmetric_ | CACACAAAAGAUGCAAAUCUUAGCGUC |
| | AUUAGU-UGUCCC | GAUCCACAGGGAGGGGGCAUUUCAAU |
| | 3_1-1_mismatch_ | AUAUCUCUGAUGUCCCCAUC |
| | A-C | |
| | 26_6-6_internal_ | |
| | loop-symmetric_ | |
| | CUAUAG-AGCGUC | |
| 283 | -11_6-6_internal_ | GAUAUUGUGACAUAUCUCUGCACUCAU |
| | loop-symmetric_ | CACACAAAAGAUGCAAAUCUUGCAACA |
| | AUUAGU-UUUCAA | GAUCCACAGGGAGGGGGCAUUCUAAU |
| | 4_1-1_mismatch_ | AUAUCUCUGAUUUCAACAUC |
| | A-C | |
| | 26_6-6_internal_ | |
| | loop-symmetric_ | |
| | CUAUAG-GCAACA | |
| 284 | -11_6-6_internal_ | GAUAUUGUGACAUAUCUCUGCACUCAU |
| | loop-symmetric_ | CACACAAAAGAUGCAAAUCUUGGACAC |
| | AUUAGU-UGGCCG | GAUCCACAGGGAGGGGGCAUCUUAAU |
| | 5_1-1_mismatch_ | AUAUCUCUGAUGGCCGCAUC |
| | A-C | |
| | 26_6-6_internal_ | |
| | loop-symmetric_ | |
| | CUAUAG-GGACAC | |
| 285 | -11_6-6_internal_ | GAUAUUGUGACAUAUCUCUGCACUCAU |
| | loop-symmetric_ | CACACAAAAGAUGCAAAUCUUAUCAUA |
| | AUUAGU-CCAGCG | GAUCCACAGGGAGGGGGCAUUUUAAU |
| | 26_6-6_internal_ | AUAUCUCUGACCAGCGCAUC |
| | loop-symmetric_ | |
| | CUAUAG-AUCAUA | |
| 286 | -11_6-6_internal_ | GAUAUUGUGACAUAUCUCUGCACUCAU |
| | loop-symmetric_ | CACACAAAAGAUGCAAAUCUGGGAAU |
| | AUUAGU-UACCGC | GGAUCCACAGGGAGGGGGCAUUUUAA |
| | 0_1-1_mismatch_ | CAUAUCUCUGAUACCGCCAUC |
| | A-C | |
| | 27_6-6_internal_ | |
| | loop-symmetric_ | |
| | UAUAGA-GGGAAU | |
| 287 | -11_6-6_internal_ | GAUAUUGUGACAUAUCUCUGCACUCAU |
| | loop-symmetric_ | CACACAAAAGAUGCAAAUCUAUGGAU |
| | AUUAGU-UAGUUG | GGAUCCACAGGGAGGGGGCAUUUCAA |
| | 3_1-1_mismatch_ | UAUAUCUCUGAUAGUUGCAUC |
| | A-C | |
| | 27_6-6_internal_ | |
| | loop-symmetric_ | |
| | UAUAGA-AUGGAU | |
| 288 | -11_6-6_internal_ | GAUAUUGUGACAUAUCUCUGCACUCAU |
| | loop-symmetric_ | CACACAAAAGAUGCAAAUCUCACGAUG |
| | AUUAGU-CAUCGG | GAUCCACAGGGAGGGGGCAUUCUAAU |
| | 4_1-1_mismatch_ | AUAUCUCUGACAUCGGCAUC |
| | A-C | |
| | 27_6-6_internal_ | |
| | loop-symmetric_ | |
| | UAUAGA-CACGAU | |
| 289 | -11_6-6_internal_ | GAUAUUGUGACAUAUCUCUGCACUCAU |
| | loop-symmetric_ | CACACAAAAGAUGCAAAUCUGGGUUU |
| | AUUAGU-UUCCCG | GGAUCCACAGGGAGGGGGCAUCUUAA |
| | 5_1-1_mismatch_ | UAUAUCUCUGAUUCCCGCAUC |
| | A-C | |
| | 27_6-6_internal_ | |
| | loop-symmetric_ | |
| | UAUAGA-GGGUUU | |
| 290 | -11_6-6_internal_ | GAUAUUGUGACAUAUCUCUGCACUCAU |
| | loop-symmetric_ | CACACAAAAGAUGCAAAUCUCAAUCCG |
| | AUUAGU-CGAUUA | GAUCCACAGGGAGGGGGCAUUUUAAU |
| | 27_6-6_internal_ | AUAUCUCUGACGAUUACAUC |
| | loop-symmetric_ | |
| | UAUAGA-CAAUCC | |
| 291 | -11_6-6_internal_ | GAUAUUGUGACAUAUCUCUGCACUCAU |
| | loop-symmetric_ | CACACAAAAGAUGCAAAUCCCUCCAAG |
| | AUUAGU-UGAGCA | GAUCCACAGGGAGGGGGCAUUUUAAC |
| | 0_1-1_mismatch_ | AUAUCUCUGAUGAGCACAUC |
| | A-C | |
| | 28_6-6_internal_ | |
| | loop-symmetric_ | |
| | AUAGAA-CCUCCA | |
| 292 | -11_6-6_internal_ | GAUAUUGUGACAUAUCUCUGCACUCAU |
| | loop-symmetric_ | CACACAAAAGAUGCAAAUCCUGGUGAG |
| | AUUAGU-UUCGAG | GAUCCACAGGGAGGGGGCAUUUCAAU |
| | 3_1-1_mismatch_ | AUAUCUCUGAUUCGAGCAUC |
| | A-C | |
| | 28_6-6_internal_ | |
| | loop-symmetric_ | |
| | AUAGAA-CUGGUG | |
| 293 | -11_6-6_internal_ | GAUAUUGUGACAUAUCUCUGCACUCAU |
| | loop-symmetric_ | CACACAAAAGAUGCAAAUCCAACGCAG |
| | AUUAGU-CCAAGA | GAUCCACAGGGAGGGGGCAUUCUAAU |
| | 4_1-1_mismatch_ | AUAUCUCUGACCAAGACAUC |
| | A-C | |
| | 28_6-6_internal_ | |
| | loop-symmetric_ | |
| | AUAGAA-CAACGC | |
| 294 | -11_6-6_internal_ | GAUAUUGUGACAUAUCUCUGCACUCAU |
| | loop-symmetric_ | CACACAAAAGAUGCAAAUCAAAAAAA |
| | AUUAGU-UUACCC | GGAUCCACAGGGAGGGGGCAUCUUAA |
| | 5_1-1_mismatch_ | UAUAUCUCUGAUUACCCCAUC |
| | A-C | |
| | 28_6-6_internal_ | |
| | loop-symmetric_ | |
| | AUAGAA-AAAAAA | |
| 295 | -11_6-6_internal_ | GAUAUUGUGACAUAUCUCUGCACUCAU |
| | loop-symmetric_ | CACACAAAAGAUGCAAAUCAGUAGCAG |
| | AUUAGU-UCCUCA | GAUCCACAGGGAGGGGGCAUUUUAAU |
| | 28_6-6_internal_ | AUAUCUCUGAUCCUCACAUC |
| | loop-symmetric_ | |
| | AUAGAA-AGUAGC | |
| 296 | -11_6-6_internal_ | GAUAUUGUGACAUAUCUCUGCACUCAU |
| | loop-symmetric_ | CACACAAAAGAUGCAAAUAAGUACUA |
| | AUUAGU-UGGAGG | GGAUCCACAGGGAGGGGGCAUUUUAA |
| | 0_1-1_mismatch_ | CAUAUCUCUGAUGGAGGCAUC |
| | A-C | |
| | 29_6-6_internal_ | |
| | loop-symmetric_ | |
| | UAGAAG-AAGUAC | |
| 297 | -11_6-6_internal_ | GAUAUUGUGACAUAUCUCUGCACUCAU |
| | loop-symmetric_ | CACACAAAAGAUGCAAAUGCGAAUUA |
| | AUUAGU-CAGCAC | GGAUCCACAGGGAGGGGGCAUUUCAA |
| | 3_1-1_mismatch_ | UAUAUCUCUGACAGCACCAUC |
| | A-C | |
| | 29_6-6_internal_ | |
| | loop-symmetric_ | |
| | UAGAAG-GCGAAU | |
| 298 | -11_6-6_internal_ | GAUAUUGUGACAUAUCUCUGCACUCAU |
| | loop-symmetric_ | CACACAAAAGAUGCAAAUGAACAUUA |
| | AUUAGU-UUGUCG | GGAUCCACAGGGAGGGGGCAUUCUAA |
| | 4_1-1_mismatch_ | UAUAUCUCUGAUUGUCGCAUC |
| | A-C | |
| | 29_6-6_internal_ | |
| | loop-symmetric_ | |
| | UAGAAG-GAACAU | |
| 299 | -11_6-6_internal_ | GAUAUUGUGACAUAUCUCUGCACUCAU |
| | loop-symmetric_ | CACACAAAAGAUGCAAAUAUCAAUUA |
| | AUUAGU-UGUCCA | GGAUCCACAGGGAGGGGGCAUUUUAA |
| | 29_6-6_internal_ | UAUAUCUCUGAUGUCCACAUC |
| | loop-symmetric_ | |
| | UAGAAG-AUCAAU | |
| 300 | -11_6-6_internal_ | GAUAUUGUGACAUAUCUCUGCACUCAU |
| | loop-symmetric_ | CACACAAAAGAUGCAAAAUAUAGAUA |
| | AUUAGU-UAGGCC | GGAUCCACAGGGAGGGGGCAUUUUAA |
| | 0_1-1_mismatch_ | CAUAUCUCUGAUAGGCCCAUC |
| | A-C | |
| | 30_6-6_internal_ | |
| | loop-symmetric_ | |
| | AGAAGA-AUAUAG | |
| 301 | -11_6-6_internal_ | GAUAUUGUGACAUAUCUCUGCACUCAU |
| | loop-symmetric_ | CACACAAAAGAUGCAAAGUACCAAUAG |
| | AUUAGU-UGUUCG | GAUCCACAGGGAGGGGGCAUUUCAAU |
| | 3_1-1_mismatch_ | AUAUCUCUGAUGUUCGCAUC |
| | A-C | |
| | 30_6-6_internal_ | |
| | loop-symmetric_ | |
| | AGAAGA-GUACCA | |
| 302 | -11_6-6_internal_ | GAUAUUGUGACAUAUCUCUGCACUCAU |
| | loop-symmetric_ | CACACAAAAGAUGCAAACGUAAAAUA |
| | AUUAGU-CAUUCC | GGAUCCACAGGGAGGGGGCAUUCUAA |
| | 4_1-1_mismatch_ | UAUAUCUCUGACAUUCCCAUC |
| | A-C | |
| | 30_6-6_internal_ | |
| | loop-symmetric_ | |
| | AGAAGA-CGUAAA | |
| 303 | -11_6-6_internal_ | GAUAUUGUGACAUAUCUCUGCACUCAU |
| | loop-symmetric_ | CACACAAAAGAUGCAAACAAGAAAUA |
| | AUUAGU-UGUCAC | GGAUCCACAGGGAGGGGGCAUCUUAA |
| | 5_1-1_mismatch_ | UAUAUCUCUGAUGUCACCAUC |
| | A-C | |
| | 30_6-6_internal_ | |
| | loop-symmetric_ | |
| | AGAAGA-CAAGAA | |
| 304 | -11_6-6_internal_ | GAUAUUGUGACAUAUCUCUGCACUCAU |
| | loop-symmetric_ | CACACAAAAGAUGCAAAAGGGGGAUA |
| | AUUAGU-UCCAGG | GGAUCCACAGGGAGGGGGCAUUUUAA |
| | 30_6-6_internal_ | UAUAUCUCUGAUCCAGGCAUC |
| | loop-symmetric_ | |
| | AGAAGA-AGGGGG | |
| 305 | -11_6-6_internal_ | GAUAUUGUGACAUAUCUCUGCACUCAU |
| | loop-symmetric_ | CACACAAAAGAUGCAACAGCGAUAUAG |
| | AUUAGU-UCCCGG | GAUCCACAGGGAGGGGGCAUUUUAAC |
| | 0_1-1_mismatch_ | AUAUCUCUGAUCCCGGCAUC |
| | A-C | |
| | 31_6-6_internal_ | |
| | loop-symmetric_ | |
| | GAAGAU-CAGCGA | |
| 306 | -11_6-6_internal_ | GAUAUUGUGACAUAUCUCUGCACUCAU |
| | loop-symmetric_ | CACACAAAAGAUGCAACUACAGUAUAG |
| | AUUAGU-CAGCAG | GAUCCACAGGGAGGGGGCAUUUCAAU |
| | 3_1-1_mismatch_ | AUAUCUCUGACAGCAGCAUC |
| | A-C | |
| | 31_6-6_internal_ | |
| | loop-symmetric_ | |
| | GAAGAU-CUACAG | |
| 307 | -11_6-6_internal_ | GAUAUUGUGACAUAUCUCUGCACUCAU |
| | loop-symmetric_ | CACACAAAAGAUGCAAUGAGAGUAUA |
| | AUUAGU-CGAAAA | GGAUCCACAGGGAGGGGGCAUUCUAA |
| | 4_1-1_mismatch_ | UAUAUCUCUGACGAAAACAUC |
| | A-C | |
| | 31_6-6_internal_ | |
| | loop-symmetric_ | |
| | GAAGAU-UGAGAG | |
| 308 | -11_6-6_internal_ | GAUAUUGUGACAUAUCUCUGCACUCAU |
| | loop-symmetric_ | CACACAAAAGAUGCAACCGCAGUAUAG |
| | AUUAGU-CAGUUC | GAUCCACAGGGAGGGGGCAUCUUAAU |
| | 5_1-1_mismatch_ | AUAUCUCUGACAGUUCCAUC |
| | A-C | |
| | 31_6-6_internal_ | |
| | loop-symmetric_ | |
| | GAAGAU-CCGCAG | |
| 309 | -11_6-6_internal_ | GAUAUUGUGACAUAUCUCUGCACUCAU |
| | loop-symmetric_ | CACACAAAAGAUGCAAUGUGAGUAUA |
| | AUUAGU-CAGUCC | GGAUCCACAGGGAGGGGGCAUUUUAA |
| | 31_6-6_internal_ | UAUAUCUCUGACAGUCCCAUC |
| | loop-symmetric_ | |
| | GAAGAU-UGUGAG | |
| 310 | -11_6-6_internal_ | GAUAUUGUGACAUAUCUCUGCACUCAU |
| | loop-symmetric_ | CACACAAAAGAUGCACGCAAGCUAUAG |
| | AUUAGU-UGACAG | GAUCCACAGGGAGGGGGCAUUUUAAC |
| | 0_1-1_mismatch_ | AUAUCUCUGAUGACAGCAUC |
| | A-C | |
| | 32_6-6_internal_ | |
| | loop-symmetric_ | |
| | AAGAUU-CGCAAG | |
| 311 | -11_6-6_internal_ | GAUAUUGUGACAUAUCUCUGCACUCAU |
| | loop-symmetric_ | CACACAAAAGAUGCAUCAGUGCUAUAG |
| | AUUAGU-UUCGGA | GAUCCACAGGGAGGGGGCAUUUCAAU |
| | 3_1-1_mismatch_ | AUAUCUCUGAUUCGGACAUC |
| | A-C | |
| | 32_6-6_internal_ | |
| | loop-symmetric_ | |
| | AAGAUU-UCAGUG | |
| 312 | -11_6-6_internal_ | GAUAUUGUGACAUAUCUCUGCACUCAU |
| | loop-symmetric_ | CACACAAAAGAUGCAUCUAUGCUAUAG |
| | AUUAGU-UUAAAG | GAUCCACAGGGAGGGGGCAUUCUAAU |
| | 4_1-1_mismatch_ | AUAUCUCUGAUUAAAGCAUC |
| | A-C | |
| | 32_6-6_internal_ | |
| | loop-symmetric_ | |
| | AAGAUU-UCUAUG | |
| 313 | -11_6-6_internal_ | GAUAUUGUGACAUAUCUCUGCACUCAU |
| | loop-symmetric_ | CACACAAAAGAUGCACCAGGCCUAUAG |
| | AUUAGU-UAGCGC | GAUCCACAGGGAGGGGGCAUCUUAAU |
| | 5_1-1_mismatch_ | AUAUCUCUGAUAGCGCCAUC |
| | A-C | |
| | 32_6-6_internal_ | |
| | loop-symmetric_ | |
| | AAGAUU-CCAGGC | |
| 314 | -11_6-6_internal_ | GAUAUUGUGACAUAUCUCUGCACUCAU |
| | loop-symmetric_ | CACACAAAAGAUGCACUAUACCUAUAG |
| | AUUAGU-UAGUCC | GAUCCACAGGGAGGGGGCAUUUUAAU |
| | 32_6-6_internal_ | AUAUCUCUGAUAGUCCCAUC |
| | loop-symmetric_ | |
| | AAGAUU-CUAUAC | |
| 315 | -11_6-6_internal_ | GAUAUUGUGACAUAUCUCUGCACUCAU |
| | loop-symmetric_ | CACACAAAAGAUGCCUAGACUCUAUAG |
| | AUUAGU-CGGUAC | GAUCCACAGGGAGGGGGCAUUUUAAC |
| | 0_1-1_mismatch_ | AUAUCUCUGACGGUACCAUC |
| | A-C | |
| | 33_6-6_internal_ | |
| | loop-symmetric_ | |
| | AGAUUU-CUAGAC | |
| 316 | -11_6-6_internal_ | GAUAUUGUGACAUAUCUCUGCACUCAU |
| | loop-symmetric_ | CACACAAAAGAUGCUGCGACUCUAUAG |
| | AUUAGU-UACACG | GAUCCACAGGGAGGGGGCAUUUCAAU |
| | 3_1-1_mismatch_ | AUAUCUCUGAUACACGCAUC |
| | A-C | |
| | 33_6-6_internal_ | |
| | loop-symmetric_ | |
| | AGAUUU-UGCGAC | |
| 317 | -11_6-6_internal_ | GAUAUUGUGACAUAUCUCUGCACUCAU |
| | loop-symmetric_ | CACACAAAAGAUGCCUUGACUCUAUAG |
| | AUUAGU-UUAUGC | GAUCCACAGGGAGGGGGCAUUCUAAU |
| | 4_1-1_mismatch_ | AUAUCUCUGAUUAUGCCAUC |
| | A-C | |
| | 33_6-6_internal_ | |
| | loop-symmetric_ | |
| | AGAUUU-CUUGAC | |
| 318 | -11_6-6_internal_ | GAUAUUGUGACAUAUCUCUGCACUCAU |
| | loop-symmetric_ | CACACAAAAGAUGCCCCAAGUCUAUAG |
| | AUUAGU-CGAGGA | GAUCCACAGGGAGGGGGCAUCUUAAU |
| | 5_1-1_mismatch_ | AUAUCUCUGACGAGGACAUC |
| | A-C | |
| | 33_6-6_internal_ | |
| | loop-symmetric_ | |
| | AGAUUU-CCCAAG | |
| 319 | -11_6-6_internal_ | GAUAUUGUGACAUAUCUCUGCACUCAU |
| | loop-symmetric_ | CACACAAAAGAUGCCGUAUAUCUAUAG |
| | AUUAGU-UGCAUC | GAUCCACAGGGAGGGGGCAUUUUAAU |
| | 33_6-6_internal_ | AUAUCUCUGAUGCAUCCAUC |
| | loop-symmetric_ | |
| | AGAUUU-CGUAUA | |
| 320 | -11_6-6_internal_ | GAUAUUGUGACAUAUCUCUGCACUCAU |
| | loop-symmetric_ | CACACAAAAGAUGGUCGAAUUCUAUA |
| | AUUAGU-CAGCGA | GGAUCCACAGGGAGGGGGCAUUUCAA |
| | 3_1-1_mismatch_ | UAUAUCUCUGACAGCGACAUC |
| | A-C | |
| | 34_6-6_internal_ | |
| | loop-symmetric_ | |
| | GAUUUG-GUCGAA | |
| 321 | -11_6-6_internal_ | GAUAUUGUGACAUAUCUCUGCACUCAU |
| | loop-symmetric_ | CACACAAAAGAUGGCCCCGUUCUAUAG |
| | AUUAGU-UUAUUA | GAUCCACAGGGAGGGGGCAUUCUAAU |
| | 4_1-1_mismatch_ | AUAUCUCUGAUUAUUACAUC |
| | A-C | |
| | 34_6-6_internal_ | |
| | loop-symmetric_ | |
| | GAUUUG-GCCCCG | |
| 322 | -11_6-6_internal_ | GAUAUUGUGACAUAUCUCUGCACUCAU |
| | loop-symmetric_ | CACACAAAAGAUGAGUGAGUUCUAUA |
| | AUUAGU-UGCCUC | GGAUCCACAGGGAGGGGGCAUCUUAA |
| | 5_1-1_mismatch_ | UAUAUCUCUGAUGCCUCCAUC |
| | A-C | |
| | 34_6-6_internal_ | |
| | loop-symmetric_ | |
| | GAUUUG-AGUGAG | |
| 323 | -11_6-6_internal_ | GAUAUUGUGACAUAUCUCUGCACUCAU |
| | loop-symmetric_ | CACACAAAAGAUGGCCCGGUUCUAUAG |
| | AUUAGU-CGGCGA | GAUCCACAGGGAGGGGGCAUUUUAAU |
| | 34_6-6_internal_ | AUAUCUCUGACGGCGACAUC |
| | loop-symmetric_ | |
| | GAUUUG-GCCCGG | |
| 324 | -11_6-6_internal_ | GAUAUUGUGACAUAUCUCUGCACUCAU |
| | loop-symmetric_ | CACACAAAAGAUCUCUGACUUCUAUAG |
| | AUUAGU-CGGUUA | GAUCCACAGGGAGGGGGCAUUUUAAC |
| | 0_1-1_mismatch_ | AUAUCUCUGACGGUUACAUC |
| | A-C | |
| | 35_6-6_internal_ | |
| | loop-symmetric_ | |
| | AUUUGC-CUCUGA | |
| 325 | -11_6-6_internal_ | GAUAUUGUGACAUAUCUCUGCACUCAU |
| | loop-symmetric_ | CACACAAAAGAUAUUCCACUUCUAUAG |
| | AUUAGU-UUGCGC | GAUCCACAGGGAGGGGGCAUUUCAAU |
| | 3_1-1_mismatch_ | AUAUCUCUGAUUGCGCCAUC |
| | A-C | |
| | 35_6-6_internal_ | |
| | loop-symmetric_ | |
| | AUUUGC-AUUCCA | |
| 326 | -11_6-6_internal_ | GAUAUUGUGACAUAUCUCUGCACUCAU |
| | loop-symmetric_ | CACACAAAAGAUCUGUUCCUUCUAUAG |
| | AUUAGU-UUACAC | GAUCCACAGGGAGGGGGCAUUCUAAU |
| | 4_1-1_mismatch_ | AUAUCUCUGAUUACACCAUC |
| | A-C | |
| | 35_6-6_internal_ | |
| | loop-symmetric_ | |
| | AUUUGC-CUGUUC | |
| 327 | -11_6-6_internal_ | GAUAUUGUGACAUAUCUCUGCACUCAU |
| | loop-symmetric_ | CACACAAAAGAUAAUUGCCUUCUAUAG |
| | AUUAGU-CAACGA | GAUCCACAGGGAGGGGGCAUUUUAAU |
| | 35_6-6_internal_ | AUAUCUCUGACAACGACAUC |
| | loop-symmetric_ | |
| | AUUUGC-AAUUGC | |
| 328 | -11_6-6_internal_ | GAUAUUGUGACAUAUCUCUGCACUCAU |
| | loop-symmetric_ | CACACAAAAGAACUUUCUCUUCUAUAG |
| | AUUAGU-UGUUCC | GAUCCACAGGGAGGGGGCAUUUCAAU |
| | 3_1-1_mismatch_ | AUAUCUCUGAUGUUCCCAUC |
| | A-C | |
| | 36_6-6_internal_ | |
| | loop-symmetric_ | |
| | UUUGCA-ACUUUC | |
| 329 | -11_6-6_internal_ | GAUAUUGUGACAUAUCUCUGCACUCAU |
| | loop-symmetric_ | CACACAAAAGAGUCCUCUCUUCUAUAG |
| | AUUAGU-UAGCAA | GAUCCACAGGGAGGGGGCAUUCUAAU |
| | 4_1-1_mismatch_ | AUAUCUCUGAUAGCAACAUC |
| | A-C | |
| | 36_6-6_internal_ | |
| | loop-symmetric_ | |
| | UUUGCA-GUCCUC | |
| 330 | -11_6-6_internal_ | GAUAUUGUGACAUAUCUCUGCACUCAU |
| | loop-symmetric_ | CACACAAAAGACAGUGUUCUUCUAUAG |
| | AUUAGU-UAAUCC | GAUCCACAGGGAGGGGGCAUCUUAAU |
| | 5_1-1_mismatch_ | AUAUCUCUGAUAAUCCCAUC |
| | A-C | |
| | 36_6-6_internal_ | |
| | loop-symmetric_ | |
| | UUUGCA-CAGUGU | |
| 331 | -11_6-6_internal_ | GAUAUUGUGACAUAUCUCUGCACUCAU |
| | loop-symmetric_ | CACACAAAAGAGAGGUUUCUUCUAUA |
| | AUUAGU-UAUCCA | GGAUCCACAGGGAGGGGGCAUUUUAA |
| | 36_6-6_internal_ | UAUAUCUCUGAUAUCCACAUC |
| | loop-symmetric_ | |
| | UUUGCA-GAGGUU | |
| 332 | -11_6-6_internal_ | GAUAUUGUGACAUAUCUCUGCACUCAU |
| | loop-symmetric_ | CACACAAAAGUCUAACAUCUUCUAUAG |
| | AUUAGU-UGCAUC | GAUCCACAGGGAGGGGGCAUUUUAAC |
| | 0_1-1_mismatch_ | AUAUCUCUGAUGCAUCCAUC |
| | A-C | |
| | 37_6-6_internal_ | |
| | loop-symmetric_ | |
| | UUGCAU-UCUAAC | |
| 333 | -11_6-6_internal_ | GAUAUUGUGACAUAUCUCUGCACUCAU |
| | loop-symmetric_ | CACACAAAAGCCUUUUAUCUUCUAUAG |
| | AUUAGU-CGGUUA | GAUCCACAGGGAGGGGGCAUUUCAAU |
| | 3_1-1_mismatch_ | AUAUCUCUGACGGUUACAUC |
| | A-C | |
| | 37_6-6_internal_ | |
| | loop-symmetric_ | |
| | UUGCAU-CCUUUU | |
| 334 | -11_6-6_internal_ | GAUAUUGUGACAUAUCUCUGCACUCAU |
| | loop-symmetric_ | CACACAAAAGUCAAUCAUCUUCUAUAG |
| | AUUAGU-UUCUUC | GAUCCACAGGGAGGGGGCAUUCUAAU |
| | 4_1-1_mismatch_ | AUAUCUCUGAUUCUUCCAUC |
| | A-C | |
| | 37_6-6_internal_ | |
| | loop-symmetric_ | |
| | UUGCAU-UCAAUC | |
| 335 | -11_6-6_internal_ | GAUAUUGUGACAUAUCUCUGCACUCAU |
| | loop-symmetric_ | CACACAAAAGUUAACUAUCUUCUAUAG |
| | AUUAGU-CAUUCC | GAUCCACAGGGAGGGGGCAUCUUAAU |
| | 5_1-1_mismatch_ | AUAUCUCUGACAUUCCCAUC |
| | A-C | |
| | 37_6-6_internal_ | |
| | loop-symmetric_ | |
| | UUGCAU-UUAACU | |
| 336 | -11_6-6_internal_ | GAUAUUGUGACAUAUCUCUGCACUCAU |
| | loop-symmetric_ | CACACAAAAGCACCCUAUCUUCUAUAG |
| | AUUAGU-CAAGGG | GAUCCACAGGGAGGGGGCAUUUUAAU |
| | 37_6-6_internal_ | AUAUCUCUGACAAGGGCAUC |
| | loop-symmetric_ | |
| | UUGCAU-CACCCU | |
| 337 | -11_6-6_internal_ | GAUAUUGUGACAUAUCUCUGCACUCAU |
| | loop-symmetric_ | CACACAAAACCAAGUAAUCUUCUAUAG |
| | AUUAGU-UCCAUA | GAUCCACAGGGAGGGGGCAUUUUAAC |
| | 0_1-1_mismatch_ | AUAUCUCUGAUCCAUACAUC |
| | A-C | |
| | 38_6-6_internal_ | |
| | loop-symmetric_ | |
| | UGCAUC-CCAAGU | |
| 338 | -11_6-6_internal_ | GAUAUUGUGACAUAUCUCUGCACUCAU |
| | loop-symmetric_ | CACACAAAACUACUCAAUCUUCUAUAG |
| | AUUAGU-CCCGCC | GAUCCACAGGGAGGGGGCAUUUCAAU |
| | 3_1-1_mismatch_ | AUAUCUCUGACCCGCCCAUC |
| | A-C | |
| | 38_6-6_internal_ | |
| | loop-symmetric_ | |
| | UGCAUC-CUACUC | |
| 339 | -11_6-6_internal_ | GAUAUUGUGACAUAUCUCUGCACUCAU |
| | loop-symmetric_ | CACACAAAACUACCUAAUCUUCUAUAG |
| | AUUAGU-UAAGUA | GAUCCACAGGGAGGGGGCAUUCUAAU |
| | 4_1-1_mismatch_ | AUAUCUCUGAUAAGUACAUC |
| | A-C | |
| | 38_6-6_internal_ | |
| | loop-symmetric_ | |
| | UGCAUC-CUACCU | |
| 340 | -11_6-6_internal_ | GAUAUUGUGACAUAUCUCUGCACUCAU |
| | loop-symmetric_ | CACACAAAAUUUAUCAAUCUUCUAUAG |
| | AUUAGU-CAGGCA | GAUCCACAGGGAGGGGGCAUCUUAAU |
| | 5_1-1_mismatch_ | AUAUCUCUGACAGGCACAUC |
| | A-C | |
| | 38_6-6_internal_ | |
| | loop-symmetric_ | |
| | UGCAUC-UUUAUC | |
| 341 | -11_6-6_internal_ | GAUAUUGUGACAUAUCUCUGCACUCAU |
| | loop-symmetric_ | CACACAAAAAUCUAUAAUCUUCUAUAG |
| | AUUAGU-UUCUUG | GAUCCACAGGGAGGGGGCAUUUUAAU |
| | 38_6-6_internal_ | AUAUCUCUGAUUCUUGCAUC |
| | loop-symmetric_ | |
| | UGCAUC-AUCUAU | |
| 342 | -11_6-6_internal_ | GAUAUUGUGACAUAUCUCUGCACUCAU |
| | loop-symmetric_ | CACACAAACUUAGAAAAUCUUCUAUAG |
| | AUUAGU-UGGGCC | GAUCCACAGGGAGGGGGCAUUUUAAC |
| | 0_1-1_mismatch_ | AUAUCUCUGAUGGGCCCAUC |
| | A-C | |
| | 39_6-6_internal_ | |
| | loop-symmetric_ | |
| | GCAUCU-CUUAGA | |
| 343 | -11_6-6_internal_ | GAUAUUGUGACAUAUCUCUGCACUCAU |
| | loop-symmetric_ | CACACAAAUCAAAAAAAUCUUCUAUAG |
| | AUUAGU-UUCUUG | GAUCCACAGGGAGGGGGCAUUUCAAU |
| | 3_1-1_mismatch_ | AUAUCUCUGAUUCUUGCAUC |
| | A-C | |
| | 39_6-6_internal_ | |
| | loop-symmetric_ | |
| | GCAUCU-UCAAAA | |
| 344 | -11_6-6_internal_ | GAUAUUGUGACAUAUCUCUGCACUCAU |
| | loop-symmetric_ | CACACAAAUCGCCAAAAUCUUCUAUAG |
| | AUUAGU-UUACGA | GAUCCACAGGGAGGGGGCAUUCUAAU |
| | 4_1-1_mismatch_ | AUAUCUCUGAUUACGACAUC |
| | A-C | |
| | 39_6-6_internal_ | |
| | loop-symmetric_ | |
| | GCAUCU-UCGCCA | |
| 345 | -11_6-6_internal_ | GAUAUUGUGACAUAUCUCUGCACUCAU |
| | loop-symmetric_ | CACACAAACUAACGAAAUCUUCUAUAG |
| | AUUAGU-UCAUGC | GAUCCACAGGGAGGGGGCAUCUUAAU |
| | 5_1-1_mismatch_ | AUAUCUCUGAUCAUGCCAUC |
| | A-C | |
| | 39_6-6_internal_ | |
| | loop-symmetric_ | |
| | GCAUCU-CUAACG | |
| 346 | -11_6-6_internal_ | GAUAUUGUGACAUAUCUCUGCACUCAU |
| | loop-symmetric_ | CACACAAAUCAGUAAAAUCUUCUAUAG |
| | AUUAGU-CAACCC | GAUCCACAGGGAGGGGGCAUUUUAAU |
| | 39_6-6_internal_ | AUAUCUCUGACAACCCCAUC |
| | loop-symmetric_ | |
| | GCAUCU-UCAGUA | |
| 347 | -11_6-6_internal_ | GAUAUUGUGACAUAUCUCUGCACUCAU |
| | loop-symmetric_ | CACACAACAUUUUCAAAUCUUCUAUAG |
| | AUUAGU-UACCUG | GAUCCACAGGGAGGGGGCAUUUUAAC |
| | 0_1-1_mismatch_ | AUAUCUCUGAUACCUGCAUC |
| | A-C | |
| | 40_6-6_internal_ | |
| | loop-symmetric_ | |
| | CAUCUU-CAUUUU | |
| 348 | -11_6-6_internal_ | GAUAUUGUGACAUAUCUCUGCACUCAU |
| | loop-symmetric_ | CACACAACACCUUCAAAUCUUCUAUAG |
| | AUUAGU-CAGUCG | GAUCCACAGGGAGGGGGCAUUUCAAU |
| | 3_1-1_mismatch_ | AUAUCUCUGACAGUCGCAUC |
| | A-C | |
| | 40_6-6_internal_ | |
| | loop-symmetric_ | |
| | CAUCUU-CACCUU | |
| 349 | -11_6-6_internal_ | GAUAUUGUGACAUAUCUCUGCACUCAU |
| | loop-symmetric_ | CACACAACCAAAACAAAUCUUCUAUAG |
| | AUUAGU-UUCUUC | GAUCCACAGGGAGGGGGCAUUCUAAU |
| | 4_1-1_mismatch_ | AUAUCUCUGAUUCUUCCAUC |
| | A-C | |
| | 40_6-6_internal_ | |
| | loop-symmetric_ | |
| | CAUCUU-CCAAAA | |
| 350 | -11_6-6_internal_ | GAUAUUGUGACAUAUCUCUGCACUCAU |
| | loop-symmetric_ | CACACAAUUCAACCAAAUCUUCUAUAG |
| | AUUAGU-CGGACA | GAUCCACAGGGAGGGGGCAUCUUAAU |
| | 5_1-1_mismatch_ | AUAUCUCUGACGGACACAUC |
| | A-C | |
| | 40_6-6_internal_ | |
| | loop-symmetric_ | |
| | CAUCUU-UUCAAC | |
| 351 | -11_6-6_internal_ | GAUAUUGUGACAUAUCUCUGCACUCAU |
| | loop-symmetric_ | CACACAAUAAUCCCAAAUCUUCUAUAG |
| | AUUAGU-UUCGGA | GAUCCACAGGGAGGGGGCAUUUUAAU |
| | 40_6-6_internal_ | AUAUCUCUGAUUCGGACAUC |
| | loop-symmetric_ | |
| | CAUCUU-UAAUCC | |
| 352 | -11_6-6_internal_ | GAUAUUGUGACAUAUCUCUGCACUCAU |
| | loop-symmetric_ | CACACAUUCCCCGCAAAUCUUCUAUAG |
| | AUUAGU-UAAGUC | GAUCCACAGGGAGGGGGCAUUUUAAC |
| | 0_1-1_mismatch_ | AUAUCUCUGAUAAGUCCAUC |
| | A-C | |
| | 41_6-6_internal_ | |
| | loop-symmetric_ | |
| | AUCUUU-UUCCCC | |
| 353 | -11_6-6_internal_ | GAUAUUGUGACAUAUCUCUGCACUCAU |
| | loop-symmetric_ | CACACAUCGUGCGCAAAUCUUCUAUAG |
| | AUUAGU-CAGAGG | GAUCCACAGGGAGGGGGCAUUUCAAU |
| | 3_1-1_mismatch_ | AUAUCUCUGACAGAGGCAUC |
| | A-C | |
| | 41_6-6_internal_ | |
| | loop-symmetric_ | |
| | AUCUUU-UCGUGC | |
| 354 | -11_6-6_internal_ | GAUAUUGUGACAUAUCUCUGCACUCAU |
| | loop-symmetric_ | CACACAUAGUAAGCAAAUCUUCUAUAG |
| | AUUAGU-UUCAAA | GAUCCACAGGGAGGGGGCAUCUUAAU |
| | 5_1-1_mismatch_ | AUAUCUCUGAUUCAAACAUC |
| | A-C | |
| | 41_6-6_internal_ | |
| | loop-symmetric_ | |
| | AUCUUU-UAGUAA | |
| 355 | -11_6-6_internal_ | GAUAUUGUGACAUAUCUCUGCACUCAU |
| | loop-symmetric_ | CACACAUUGUCGGCAAAUCUUCUAUAG |
| | AUUAGU-UCACGG | GAUCCACAGGGAGGGGGCAUUUUAAU |
| | 41_6-6_internal_ | AUAUCUCUGAUCACGGCAUC |
| | loop-symmetric_ | |
| | AUCUUU-UUGUCG | |
| 356 | -11_6-6_internal_ | GAUAUUGUGACAUAUCUCUGCACUCAU |
| | loop-symmetric_ | CACACUCCUUUUGCAAAUCUUCUAUAG |
| | AUUAGU-CGACCG | GAUCCACAGGGAGGGGGCAUUUUAAC |
| | 0_1-1_mismatch_ | AUAUCUCUGACGACCGCAUC |
| | A-C | |
| | 42_6-6_internal_ | |
| | loop-symmetric_ | |
| | UCUUUU-UCCUUU | |
| 357 | -11_6-6_internal_ | GAUAUUGUGACAUAUCUCUGCACUCAU |
| | loop-symmetric_ | CACACCCACUUUGCAAAUCUUCUAUAG |
| | AUUAGU-UUCAUA | GAUCCACAGGGAGGGGGCAUUUCAAU |
| | 3_1-1_mismatch_ | AUAUCUCUGAUUCAUACAUC |
| | A-C | |
| | 42_6-6_internal_ | |
| | loop-symmetric_ | |
| | UCUUUU-CCACUU | |
| 358 | -11_6-6_internal_ | GAUAUUGUGACAUAUCUCUGCACUCAU |
| | loop-symmetric_ | CACACUCCCUCUGCAAAUCUUCUAUAG |
| | AUUAGU-CCCUCG | GAUCCACAGGGAGGGGGCAUUCUAAU |
| | 4_1-1_mismatch_ | AUAUCUCUGACCCUCGCAUC |
| | A-C | |
| | 42_6-6_internal_ | |
| | loop-symmetric_ | |
| | UCUUUU-UCCCUC | |
| 359 | -11_6-6_internal_ | GAUAUUGUGACAUAUCUCUGCACUCAU |
| | loop-symmetric_ | CACACUUCACCUGCAAAUCUUCUAUAG |
| | AUUAGU-UAGCAC | GAUCCACAGGGAGGGGGCAUCUUAAU |
| | 5_1-1_mismatch_ | AUAUCUCUGAUAGCACCAUC |
| | A-C | |
| | 42_6-6_internal_ | |
| | loop-symmetric_ | |
| | UCUUUU-UUCACC | |
| 360 | -11_6-6_internal_ | GAUAUUGUGACAUAUCUCUGCACUCAU |
| | loop-symmetric_ | CACACUAUCAUUGCAAAUCUUCUAUAG |
| | AUUAGU-UGCAUA | GAUCCACAGGGAGGGGGCAUUUUAAU |
| | 42_6-6_internal_ | AUAUCUCUGAUGCAUACAUC |
| | loop-symmetric_ | |
| | UCUUUU-UAUCAU | |
| 361 | -11_6-6_internal_ | GAUAUUGUGACAUAUCUCUGCACUCAU |
| | loop-symmetric_ | CACAGUCCAUAUGCAAAUCUUCUAUAG |
| | AUUAGU-UGCAUA | GAUCCACAGGGAGGGGGCAUUUUAAC |
| | 0_1-1_mismatch_ | AUAUCUCUGAUGCAUACAUC |
| | A-C | |
| | 43_6-6_internal_ | |
| | loop-symmetric_ | |
| | CUUUUG-GUCCAU | |
| 362 | -11_6-6_internal_ | GAUAUUGUGACAUAUCUCUGCACUCAU |
| | loop-symmetric_ | CACAAAUCCCAUGCAAAUCUUCUAUAG |
| | AUUAGU-CCAUGC | GAUCCACAGGGAGGGGGCAUUCUAAU |
| | 4_1-1_mismatch_ | AUAUCUCUGACCAUGCCAUC |
| | A-C | |
| | 43_6-6_internal_ | |
| | loop-symmetric_ | |
| | CUUUUG-AAUCCC | |
| 363 | -11_6-6_internal_ | GAUAUUGUGACAUAUCUCUGCACUCAU |
| | loop-symmetric_ | CACAAGCAUUAUGCAAAUCUUCUAUAG |
| | AUUAGU-UGCAGC | GAUCCACAGGGAGGGGGCAUCUUAAU |
| | 5_1-1_mismatch_ | AUAUCUCUGAUGCAGCCAUC |
| | A-C | |
| | 43_6-6_internal_ | |
| | loop-symmetric_ | |
| | CUUUUG-AGCAUU | |
| 364 | -11_6-6_internal_ | GAUAUUGUGACAUAUCUCUGCACUCAU |
| | loop-symmetric_ | CACAACCCCAAUGCAAAUCUUCUAUAG |
| | AUUAGU-CAUCGG | GAUCCACAGGGAGGGGGCAUUUUAAU |
| | 43_6-6_internal_ | AUAUCUCUGACAUCGGCAUC |
| | loop-symmetric_ | |
| | CUUUUG-ACCCCA | |
| 365 | -11_6-6_internal_ | GAUAUUGUGACAUAUCUCUGCACUCAU |
| | loop-symmetric_ | CACUAACCUGAUGCAAAUCUUCUAUAG |
| | AUUAGU-CCAAAG | GAUCCACAGGGAGGGGGCAUUUUAAC |
| | 0_1-1_mismatch_ | AUAUCUCUGACCAAAGCAUC |
| | A-C | |
| | 44_6-6_internal_ | |
| | loop-symmetric_ | |
| | UUUUGU-UAACCU | |
| 366 | -11_6-6_internal_ | GAUAUUGUGACAUAUCUCUGCACUCAU |
| | loop-symmetric_ | CACCUGGUCGAUGCAAAUCUUCUAUAG |
| | AUUAGU-UAAGCC | GAUCCACAGGGAGGGGGCAUUUCAAU |
| | 3_1-1_mismatch_ | AUAUCUCUGAUAAGCCCAUC |
| | A-C | |
| | 44_6-6_internal_ | |
| | loop-symmetric_ | |
| | UUUUGU-CUGGUC | |
| 367 | -11_6-6_internal_ | GAUAUUGUGACAUAUCUCUGCACUCAU |
| | loop-symmetric_ | CACCUCUUUGAUGCAAAUCUUCUAUAG |
| | AUUAGU-CCAACC | GAUCCACAGGGAGGGGGCAUUCUAAU |
| | 4_1-1_mismatch_ | AUAUCUCUGACCAACCCAUC |
| | A-C | |
| | 44_6-6_internal_ | |
| | loop-symmetric_ | |
| | UUUUGU-CUCUUU | |
| 368 | -11_6-6_internal_ | GAUAUUGUGACAUAUCUCUGCACUCAU |
| | loop-symmetric_ | CACCUCUACGAUGCAAAUCUUCUAUAG |
| | AUUAGU-CGGCCC | GAUCCACAGGGAGGGGGCAUCUUAAU |
| | 5_1-1_mismatch_ | AUAUCUCUGACGGCCCCAUC |
| | A-C | |
| | 44_6-6_internal_ | |
| | loop-symmetric_ | |
| | UUUUGU-CUCUAC | |
| 369 | -11_6-6_internal_ | GAUAUUGUGACAUAUCUCUGCACUCAU |
| | loop-symmetric_ | CACUUGUGUGAUGCAAAUCUUCUAUA |
| | AUUAGU-CAUUCC | GGAUCCACAGGGAGGGGGCAUUUUAA |
| | 44_6-6_internal_ | UAUAUCUCUGACAUUCCCAUC |
| | loop-symmetric_ | |
| | UUUUGU-UUGUGU | |
| 370 | -10_6-6_internal_ | GAUAUUGUGACAUAUCUCUGCACUCAU |
| | loop-symmetric_ | CACACAAAAGAUGCAAAUCUUCUAUAG |
| | UUAGUU-CCCAGC | GAUCCACAGGGAGGGGGCAUUUUAAC |
| | 0_1-1_mismatch_ | AUAUCUCUGCCCAGCUCAUC |
| | A-C | |
| 371 | -10_6-6_internal_ | GAUAUUGUGACAUAUCUCUGCACUCAU |
| | loop-symmetric_ | CACACAAAAGAUGCAAAUCUUCUAUAG |
| | UUAGUU-UUUCUC | GAUCCACAGGGAGGGGGCAUUUCAAU |
| | 3_1-1_mismatch_ | AUAUCUCUGUUUCUCUCAUC |
| | A-C | |
| 372 | -10_6-6_internal_ | GAUAUUGUGACAUAUCUCUGCACUCAU |
| | loop-symmetric_ | CACACAAAAGAUGCAAAUCUUCUAUAG |
| | UUAGUU-CCCAUC | GAUCCACAGGGAGGGGGCAUUCUAAU |
| | 4_1-1_mismatch_ | AUAUCUCUGCCCAUCUCAUC |
| | A-C | |
| 373 | -10_6-6_internal_ | GAUAUUGUGACAUAUCUCUGCACUCAU |
| | loop-symmetric_ | CACACAAAAGAUGCAAAUCUUCUAUAG |
| | UUAGUU-CCUCGC | GAUCCACAGGGAGGGGGCAUCUUAAU |
| | 5_1-1_mismatch_ | AUAUCUCUGCCUCGCUCAUC |
| | A-C | |
| 374 | -10_6-6_internal_ | GAUAUUGUGACAUAUCUCUGCACUCAU |
| | loop-symmetric_ | CACACAAAAGAUGCAAAUCUUCUAUAG |
| | UUAGUU-CGUACC | GAUCCACAGGGAGGGGGCAUUUUAAU |
| | | AUAUCUCUGCGUACCUCAUC |
| 375 | -10_6-6_internal_ | GAUAUUGUGACAUAUCUCUGCACUCAU |
| | loop-symmetric_ | CACACAAAAGAUGCAAAUCUUCUAUAG |
| | UUAGUU-UUGAUU | UGCGGUCAGGGAGGGGGCAUUUUAAC |
| | 0_1-1_mismatch_ | AUAUCUCUGUUGAUUUCAUC |
| | A-C | |
| | 20_6-6_internal_ | |
| | loop-symmetric_ | |
| | UGGAUC-UGCGGU | |
| 376 | -10_6-6_internal_ | GAUAUUGUGACAUAUCUCUGCACUCAU |
| | loop-symmetric_ | CACACAAAAGAUGCAAAUCUUCUAUAG |
| | UUAGUU-CUUUCC | UACAAUCAGGGAGGGGGCAUUUCAAU |
| | 3_1-1_mismatch_ | AUAUCUCUGCUUUCCUCAUC |
| | A-C | |
| | 20_6-6_internal_ | |
| | loop-symmetric_ | |
| | UGGAUC-UACAAU | |
| 377 | -10_6-6_internal_ | GAUAUUGUGACAUAUCUCUGCACUCAU |
| | loop-symmetric_ | CACACAAAAGAUGCAAAUCUUCUAUAG |
| | UUAGUU-UCGGGU | UCAUAUCAGGGAGGGGGCAUUCUAAU |
| | 4_1-1_mismatch_ | AUAUCUCUGUCGGGUUCAUC |
| | A-C | |
| | 20_6-6_internal_ | |
| | loop-symmetric_ | |
| | UGGAUC-UCAUAU | |
| 378 | -10_6-6_internal_ | GAUAUUGUGACAUAUCUCUGCACUCAU |
| | loop-symmetric_ | CACACAAAAGAUGCAAAUCUUCUAUAG |
| | UUAGUU-CGUACC | ACUAAUCAGGGAGGGGGCAUCUUAAU |
| | 5_1-1_mismatch_ | AUAUCUCUGCGUACCUCAUC |
| | A-C | |
| | 20_6-6_internal_ | |
| | loop-symmetric_ | |
| | UGGAUC-ACUAAU | |
| 379 | -10_6-6_internal_ | GAUAUUGUGACAUAUCUCUGCACUCAU |
| | loop-symmetric_ | CACACAAAAGAUGCAAAUCUUCUAUAG |
| | UUAGUU-CAAGAU | AACAAUCAGGGAGGGGGCAUUUUAAU |
| | 20_6-6_internal_ | AUAUCUCUGCAAGAUUCAUC |
| | loop-symmetric_ | |
| | UGGAUC-AACAAU | |
| 380 | -10_6-6_internal_ | GAUAUUGUGACAUAUCUCUGCACUCAU |
| | loop-symmetric_ | CACACAAAAGAUGCAAAUCUUCUAUAC |
| | UUAGUU-UGUUCC | AUGAAACAGGGAGGGGGCAUUUUAAC |
| | 0_1-1_mismatch_ | AUAUCUCUGUGUUCCUCAUC |
| | A-C | |
| | 21_6-6_internal_ | |
| | loop-symmetric_ | |
| | GGAUCC-CAUGAA | |
| 381 | -10_6-6_internal_ | GAUAUUGUGACAUAUCUCUGCACUCAU |
| | loop-symmetric_ | CACACAAAAGAUGCAAAUCUUCUAUAU |
| | UUAGUU-UUUGUU | CUGCAACAGGGAGGGGGCAUUUCAAU |
| | 3_1-1_mismatch_ | AUAUCUCUGUUUGUUUCAUC |
| | A-C | |
| | 21_6-6_internal_ | |
| | loop-symmetric_ | |
| | GGAUCC-UCUGCA | |
| 382 | -10_6-6_internal_ | GAUAUUGUGACAUAUCUCUGCACUCAU |
| | loop-symmetric_ | CACACAAAAGAUGCAAAUCUUCUAUAC |
| | UUAGUU-UUAACC | CCAAGACAGGGAGGGGGCAUUCUAAU |
| | 4_1-1_mismatch_ | AUAUCUCUGUUAACCUCAUC |
| | A-C | |
| | 21_6-6_internal_ | |
| | loop-symmetric_ | |
| | GGAUCC-CCCAAG | |
| 383 | -10_6-6_internal_ | GAUAUUGUGACAUAUCUCUGCACUCAU |
| | loop-symmetric_ | CACACAAAAGAUGCAAAUCUUCUAUAA |
| | UUAGUU-UCAGUC | UUGAGACAGGGAGGGGGCAUCUUAAU |
| | 5_1-1_mismatch_ | AUAUCUCUGUCAGUCUCAUC |
| | A-C | |
| | 21_6-6_internal_ | |
| | loop-symmetric_ | |
| | GGAUCC-AUUGAG | |
| 384 | -10_6-6_internal_ | GAUAUUGUGACAUAUCUCUGCACUCAU |
| | loop-symmetric_ | CACACAAAAGAUGCAAAUCUUCUAUCG |
| | UUAGUU-UGAGUU | CUAGCACAGGGAGGGGGCAUUUUAAC |
| | 0_1-1_mismatch_ | AUAUCUCUGUGAGUUUCAUC |
| | A-C | |
| | 22_6-6_internal_ | |
| | loop-symmetric_ | |
| | GAUCCU-CGCUAG | |
| 385 | -10_6-6_internal_ | GAUAUUGUGACAUAUCUCUGCACUCAU |
| | loop-symmetric_ | CACACAAAAGAUGCAAAUCUUCUAUUU |
| | UUAGUU-UUGGGC | CCAACACAGGGAGGGGGCAUUUCAAUA |
| | 3_1-1_mismatch_ | UAUCUCUGUUGGGCUCAUC |
| | A-C | |
| | 22_6-6_internal_ | |
| | loop-symmetric_ | |
| | GAUCCU-UUCCAA | |
| 386 | -10_6-6_internal_ | GAUAUUGUGACAUAUCUCUGCACUCAU |
| | loop-symmetric_ | CACACAAAAGAUGCAAAUCUUCUAUUA |
| | UUAGUU-UGAGAC | AAAGCACAGGGAGGGGGCAUUCUAAU |
| | 4_1-1_mismatch_ | AUAUCUCUGUGAGACUCAUC |
| | A-C | |
| | 22_6-6_internal_ | |
| | loop-symmetric_ | |
| | GAUCCU-UAAAAG | |
| 387 | -10_6-6_internal_ | GAUAUUGUGACAUAUCUCUGCACUCAU |
| | loop-symmetric_ | CACACAAAAGAUGCAAAUCUUCUAUCC |
| | UUAGUU-CGUAUU | CGAACACAGGGAGGGGGCAUCUUAAU |
| | 5_1-1_mismatch_ | AUAUCUCUGCGUAUUUCAUC |
| | A-C | |
| | 22_6-6_internal_ | |
| | loop-symmetric_ | |
| | GAUCCU-CCCGAA | |
| 388 | -10_6-6_internal_ | GAUAUUGUGACAUAUCUCUGCACUCAU |
| | loop-symmetric_ | CACACAAAAGAUGCAAAUCUUCUAUCA |
| | UUAGUU-CGGGAU | UGCGCACAGGGAGGGGGCAUUUUAAU |
| | 22_6-6_internal_ | AUAUCUCUGCGGGAUUCAUC |
| | loop-symmetric_ | |
| | GAUCCU-CAUGCG | |
| 389 | -10_6-6_internal_ | GAUAUUGUGACAUAUCUCUGCACUCAU |
| | loop-symmetric_ | CACACAAAAGAUGCAAAUCUUCUACCC |
| | UUAGUU-CCCACC | UGGCCACAGGGAGGGGGCAUUUUAAC |
| | 0_1-1_mismatch_ | AUAUCUCUGCCCACCUCAUC |
| | A-C | |
| | 23_6-6_internal_ | |
| | loop-symmetric_ | |
| | AUCCUA-CCCUGG | |
| 390 | -10_6-6_internal_ | GAUAUUGUGACAUAUCUCUGCACUCAU |
| | loop-symmetric_ | CACACAAAAGAUGCAAAUCUUCUAAAA |
| | UUAGUU-UCCCGC | UCCCCACAGGGAGGGGGCAUUUCAAUA |
| | 3_1-1_mismatch_ | UAUCUCUGUCCCGCUCAUC |
| | A-C | |
| | 23_6-6_internal_ | |
| | loop-symmetric_ | |
| | AUCCUA-AAAUCC | |
| 391 | -10_6-6_internal_ | GAUAUUGUGACAUAUCUCUGCACUCAU |
| | loop-symmetric_ | CACACAAAAGAUGCAAAUCUUCUACUC |
| | UUAGUU-UUAAGU | AUGCCACAGGGAGGGGGCAUUCUAAU |
| | 4_1-1_mismatch_ | AUAUCUCUGUUAAGUUCAUC |
| | A-C | |
| | 23_6-6_internal_ | |
| | loop-symmetric_ | |
| | AUCCUA-CUCAUG | |
| 392 | -10_6-6_internal_ | GAUAUUGUGACAUAUCUCUGCACUCAU |
| | loop-symmetric_ | CACACAAAAGAUGCAAAUCUUCUAAUU |
| | UUAGUU-UGUCUU | AUGCCACAGGGAGGGGGCAUCUUAAU |
| | 5_1-1_mismatch_ | AUAUCUCUGUGUCUUUCAUC |
| | A-C | |
| | 23_6-6_internal_ | |
| | loop-symmetric_ | |
| | AUCCUA-AUUAUG | |
| 393 | -10_6-6_internal_ | GAUAUUGUGACAUAUCUCUGCACUCAU |
| | loop-symmetric_ | CACACAAAAGAUGCAAAUCUUCUAGGC |
| | UUAGUU-CCACCU | CUCCCACAGGGAGGGGGCAUUUUAAUA |
| | 23_6-6_internal_ | UAUCUCUGCCACCUUCAUC |
| | loop-symmetric_ | |
| | AUCCUA-GGCCUC | |
| 394 | -10_6-6_internal_ | GAUAUUGUGACAUAUCUCUGCACUCAU |
| | loop-symmetric_ | CACACAAAAGAUGCAAAUCUUCUUCAU |
| | UUAGUU-UUACAC | CCUCCACAGGGAGGGGGCAUUUUAACA |
| | 0_1-1_mismatch_ | UAUCUCUGUUACACUCAUC |
| | A-C | |
| | 24_6-6_internal_ | |
| | loop-symmetric_ | |
| | UCCUAU-UCAUCC | |
| 395 | -10_6-6_internal_ | GAUAUUGUGACAUAUCUCUGCACUCAU |
| | loop-symmetric_ | CACACAAAAGAUGCAAAUCUUCUCCGU |
| | UUAGUU-UUGCCU | UUUCCACAGGGAGGGGGCAUUUCAAU |
| | 3_1-1_mismatch_ | AUAUCUCUGUUGCCUUCAUC |
| | A-C | |
| | 24_6-6_internal_ | |
| | loop-symmetric_ | |
| | UCCUAU-CCGUUU | |
| 396 | -10_6-6_internal_ | GAUAUUGUGACAUAUCUCUGCACUCAU |
| | loop-symmetric_ | CACACAAAAGAUGCAAAUCUUCUUGCC |
| | UUAGUU-CGGAAC | GUUCCACAGGGAGGGGGCAUUCUAAU |
| | 4_1-1_mismatch_ | AUAUCUCUGCGGAACUCAUC |
| | A-C | |
| | 24_6-6_internal_ | |
| | loop-symmetric_ | |
| | UCCUAU-UGCCGU | |
| 397 | -10_6-6_internal_ | GAUAUUGUGACAUAUCUCUGCACUCAU |
| | loop-symmetric_ | CACACAAAAGAUGCAAAUCUUCUUCCA |
| | UUAGUU-CGAUCC | AUUCCACAGGGAGGGGGCAUCUUAAU |
| | 5_1-1_mismatch_ | AUAUCUCUGCGAUCCUCAUC |
| | A-C | |
| | 24_6-6_internal_ | |
| | loop-symmetric_ | |
| | UCCUAU-UCCAAU | |
| 398 | -10_6-6_internal_ | GAUAUUGUGACAUAUCUCUGCACUCAU |
| | loop-symmetric_ | CACACAAAAGAUGCAAAUCUUCUCAUU |
| | UUAGUU-UGUCGU | UUUCCACAGGGAGGGGGCAUUUUAAU |
| | 24_6-6_internal_ | AUAUCUCUGUGUCGUUCAUC |
| | loop-symmetric_ | |
| | UCCUAU-CAUUUU | |
| 399 | -10_6-6_internal_ | GAUAUUGUGACAUAUCUCUGCACUCAU |
| | loop-symmetric_ | CACACAAAAGAUGCAAAUCUUCGCCGC |
| | UUAGUU-UCGGAC | AAUCCACAGGGAGGGGGCAUUUUAAC |
| | 0_1-1_mismatch_ | AUAUCUCUGUCGGACUCAUC |
| | A-C | |
| | 25_6-6_internal_ | |
| | loop-symmetric_ | |
| | CCUAUA-GCCGCA | |
| 400 | -10_6-6_internal_ | GAUAUUGUGACAUAUCUCUGCACUCAU |
| | loop-symmetric_ | CACACAAAAGAUGCAAAUCUUCAGCUA |
| | UUAGUU-CCAAAU | UAUCCACAGGGAGGGGGCAUUUCAAU |
| | 3_1-1_mismatch_ | AUAUCUCUGCCAAAUUCAUC |
| | A-C | |
| | 25_6-6_internal_ | |
| | loop-symmetric_ | |
| | CCUAUA-AGCUAU | |
| 401 | -10_6-6_internal_ | GAUAUUGUGACAUAUCUCUGCACUCAU |
| | loop-symmetric_ | CACACAAAAGAUGCAAAUCUUCGAAAC |
| | UUAGUU-CUGGUU | UAUCCACAGGGAGGGGGCAUUCUAAU |
| | 4_1-1_mismatch_ | AUAUCUCUGCUGGUUUCAUC |
| | A-C | |
| | 25_6-6_internal_ | |
| | loop-symmetric_ | |
| | CCUAUA-GAAACU | |
| 402 | -10_6-6_internal_ | GAUAUUGUGACAUAUCUCUGCACUCAU |
| | loop-symmetric_ | CACACAAAAGAUGCAAAUCUUCCGAUU |
| | UUAGUU-UCAAUC | CAUCCACAGGGAGGGGGCAUCUUAAUA |
| | 5_1-1_mismatch_ | UAUCUCUGUCAAUCUCAUC |
| | A-C | |
| | 25_6-6_internal_ | |
| | loop-symmetric_ | |
| | CCUAUA-CGAUUC | |
| 403 | -10_6-6_internal_ | GAUAUUGUGACAUAUCUCUGCACUCAU |
| | loop-symmetric_ | CACACAAAAGAUGCAAAUCUUGCCUCA |
| | UUAGUU-UUGGAU | GAUCCACAGGGAGGGGGCAUUUUAAC |
| | 0_1-1_mismatch_ | AUAUCUCUGUUGGAUUCAUC |
| | A-C | |
| | 26_6-6_internal_ | |
| | loop-symmetric_ | |
| | CUAUAG-GCCUCA | |
| 404 | -10_6-6_internal_ | GAUAUUGUGACAUAUCUCUGCACUCAU |
| | loop-symmetric_ | CACACAAAAGAUGCAAAUCUUAAAGCU |
| | UUAGUU-UCGUCC | GAUCCACAGGGAGGGGGCAUUUCAAU |
| | 3_1-1_mismatch_ | AUAUCUCUGUCGUCCUCAUC |
| | A-C | |
| | 26_6-6_internal_ | |
| | loop-symmetric_ | |
| | CUAUAG-AAAGCU | |
| 405 | -10_6-6_internal_ | GAUAUUGUGACAUAUCUCUGCACUCAU |
| | loop-symmetric_ | CACACAAAAGAUGCAAAUCUUACCGAA |
| | UUAGUU-CCGUCU | GAUCCACAGGGAGGGGGCAUUCUAAU |
| | 4_1-1_mismatch_ | AUAUCUCUGCCGUCUUCAUC |
| | A-C | |
| | 26_6-6_internal_ | |
| | loop-symmetric_ | |
| | CUAUAG-ACCGAA | |
| 406 | -10_6-6_internal_ | GAUAUUGUGACAUAUCUCUGCACUCAU |
| | loop-symmetric_ | CACACAAAAGAUGCAAAUCUUAUCCUC |
| | UUAGUU-UCAAGU | GAUCCACAGGGAGGGGGCAUCUUAAU |
| | 5_1-1_mismatch_ | AUAUCUCUGUCAAGUUCAUC |
| | A-C | |
| | 26_6-6_internal_ | |
| | loop-symmetric_ | |
| | CUAUAG-AUCCUC | |
| 407 | -10_6-6_internal_ | GAUAUUGUGACAUAUCUCUGCACUCAU |
| | loop-symmetric_ | CACACAAAAGAUGCAAAUCUUAGUGCA |
| | UUAGUU-CGGAUC | GAUCCACAGGGAGGGGGCAUUUUAAU |
| | 26_6-6_internal_ | AUAUCUCUGCGGAUCUCAUC |
| | loop-symmetric_ | |
| | CUAUAG-AGUGCA | |
| 408 | -10_6-6_internal_ | GAUAUUGUGACAUAUCUCUGCACUCAU |
| | loop-symmetric_ | CACACAAAAGAUGCAAAUCUAACCCCG |
| | UUAGUU-CCUCGC | GAUCCACAGGGAGGGGGCAUUUUAAC |
| | 0_1-1_mismatch_ | AUAUCUCUGCCUCGCUCAUC |
| | A-C | |
| | 27_6-6_internal_ | |
| | loop-symmetric_ | |
| | UAUAGA-AACCCC | |
| 409 | -10_6-6_internal_ | GAUAUUGUGACAUAUCUCUGCACUCAU |
| | loop-symmetric_ | CACACAAAAGAUGCAAAUCUGGGAGCG |
| | UUAGUU-UUACAU | GAUCCACAGGGAGGGGGCAUUUCAAU |
| | 3_1-1_mismatch_ | AUAUCUCUGUUACAUUCAUC |
| | A-C | |
| | 27_6-6_internal_ | |
| | loop-symmetric_ | |
| | UAUAGA-GGGAGC | |
| 410 | -10_6-6_internal_ | GAUAUUGUGACAUAUCUCUGCACUCAU |
| | loop-symmetric_ | CACACAAAAGAUGCAAAUCUCACGAUG |
| | UUAGUU-UGAGCC | GAUCCACAGGGAGGGGGCAUUCUAAU |
| | 4_1-1_mismatch_ | AUAUCUCUGUGAGCCUCAUC |
| | A-C | |
| | 27_6-6_internal_ | |
| | loop-symmetric_ | |
| | UAUAGA-CACGAU | |
| 411 | -10_6-6_internal_ | GAUAUUGUGACAUAUCUCUGCACUCAU |
| | loop-symmetric_ | CACACAAAAGAUGCAAAUCUCAUCCUG |
| | UUAGUU-CGUAUC | GAUCCACAGGGAGGGGGCAUUUUAAU |
| | 27_6-6_internal_ | AUAUCUCUGCGUAUCUCAUC |
| | loop-symmetric_ | |
| | UAUAGA-CAUCCU | |
| 412 | -10_6-6_internal_ | GAUAUUGUGACAUAUCUCUGCACUCAU |
| | loop-symmetric_ | CACACAAAAGAUGCAAAUCAAUACAAG |
| | UUAGUU-CGGAUC | GAUCCACAGGGAGGGGGCAUUUUAAC |
| | 0_1-1_mismatch_ | AUAUCUCUGCGGAUCUCAUC |
| | A-C | |
| | 28_6-6_internal_ | |
| | loop-symmetric_ | |
| | AUAGAA-AAUACA | |
| 413 | -10_6-6_internal_ | GAUAUUGUGACAUAUCUCUGCACUCAU |
| | loop-symmetric_ | CACACAAAAGAUGCAAAUCGGAGAGA |
| | UUAGUU-CCAGGC | GGAUCCACAGGGAGGGGGCAUUUCAA |
| | 3_1-1_mismatch_ | UAUAUCUCUGCCAGGCUCAUC |
| | A-C | |
| | 28_6-6_internal_ | |
| | loop-symmetric_ | |
| | AUAGAA-GGAGAG | |
| 414 | -10_6-6_internal_ | GAUAUUGUGACAUAUCUCUGCACUCAU |
| | loop-symmetric_ | CACACAAAAGAUGCAAAUCCGGCGCAG |
| | UUAGUU-CAGAUU | GAUCCACAGGGAGGGGGCAUUCUAAU |
| | 4_1-1_mismatch_ | AUAUCUCUGCAGAUUUCAUC |
| | A-C | |
| | 28_6-6_internal_ | |
| | loop-symmetric_ | |
| | AUAGAA-CGGCGC | |
| 415 | -10_6-6_internal_ | GAUAUUGUGACAUAUCUCUGCACUCAU |
| | loop-symmetric_ | CACACAAAAGAUGCAAAUCGCGCAAAG |
| | UUAGUU-UAAAGC | GAUCCACAGGGAGGGGGCAUCUUAAU |
| | 5_1-1_mismatch_ | AUAUCUCUGUAAAGCUCAUC |
| | A-C | |
| | 28_6-6_internal_ | |
| | loop-symmetric_ | |
| | AUAGAA-GCGCAA | |
| 416 | -10_6-6_internal_ | GAUAUUGUGACAUAUCUCUGCACUCAU |
| | loop-symmetric_ | CACACAAAAGAUGCAAAUCGUGGUGA |
| | UUAGUU-CCGAGC | GGAUCCACAGGGAGGGGGCAUUUUAA |
| | 28_6-6_internal_ | UAUAUCUCUGCCGAGCUCAUC |
| | loop-symmetric_ | |
| | AUAGAA-GUGGUG | |
| 417 | -10_6-6_internal_ | GAUAUUGUGACAUAUCUCUGCACUCAU |
| | loop-symmetric_ | CACACAAAAGAUGCAAAUGCAUUUUA |
| | UUAGUU-UUAAAC | GGAUCCACAGGGAGGGGGCAUUUUAA |
| | 0_1-1_mismatch_ | CAUAUCUCUGUUAAACUCAUC |
| | A-C | |
| | 29_6-6_internal_ | |
| | loop-symmetric_ | |
| | UAGAAG-GCAUUU | |
| 418 | -10_6-6_internal_ | GAUAUUGUGACAUAUCUCUGCACUCAU |
| | loop-symmetric_ | CACACAAAAGAUGCAAAUGGGAAUUA |
| | UUAGUU-UGAACC | GGAUCCACAGGGAGGGGGCAUUUCAA |
| | 3_1-1_mismatch_ | UAUAUCUCUGUGAACCUCAUC |
| | A-C | |
| | 29_6-6_internal_ | |
| | loop-symmetric_ | |
| | UAGAAG-GGGAAU | |
| 419 | -10_6-6_internal_ | GAUAUUGUGACAUAUCUCUGCACUCAU |
| | loop-symmetric_ | CACACAAAAGAUGCAAAUGCUAAUUA |
| | UUAGUU-UCACCC | GGAUCCACAGGGAGGGGGCAUUCUAA |
| | 4_1-1_mismatch_ | UAUAUCUCUGUCACCCUCAUC |
| | A-C | |
| | 29_6-6_internal_ | |
| | loop-symmetric_ | |
| | UAGAAG-GCUAAU | |
| 420 | -10_6-6_internal_ | GAUAUUGUGACAUAUCUCUGCACUCAU |
| | loop-symmetric_ | CACACAAAAGAUGCAAAUAUGGAUUA |
| | UUAGUU-CCGGUU | GGAUCCACAGGGAGGGGGCAUCUUAA |
| | 5_1-1_mismatch_ | UAUAUCUCUGCCGGUUUCAUC |
| | A-C | |
| | 29_6-6_internal_ | |
| | loop-symmetric_ | |
| | UAGAAG-AUGGAU | |
| 421 | -10_6-6_internal_ | GAUAUUGUGACAUAUCUCUGCACUCAU |
| | loop-symmetric_ | CACACAAAAGAUGCAAAUAAUACCUAG |
| | UUAGUU-UUUAUC | GAUCCACAGGGAGGGGGCAUUUUAAU |
| | 29_6-6_internal_ | AUAUCUCUGUUUAUCUCAUC |
| | loop-symmetric_ | |
| | UAGAAG-AAUACC | |
| 422 | -10_6-6_internal_ | GAUAUUGUGACAUAUCUCUGCACUCAU |
| | loop-symmetric_ | CACACAAAAGAUGCAAACAGCACAUAG |
| | UUAGUU-CUGGAU | GAUCCACAGGGAGGGGGCAUUUUAAC |
| | 0_1-1_mismatch_ | AUAUCUCUGCUGGAUUCAUC |
| | A-C | |
| | 30_6-6_internal_ | |
| | loop-symmetric_ | |
| | AGAAGA-CAGCAC | |
| 423 | -10_6-6_internal_ | GAUAUUGUGACAUAUCUCUGCACUCAU |
| | loop-symmetric_ | CACACAAAAGAUGCAAAACAAACAUAG |
| | UUAGUU-CCCCCU | GAUCCACAGGGAGGGGGCAUUUCAAU |
| | 3_1-1_mismatch_ | AUAUCUCUGCCCCCUUCAUC |
| | A-C | |
| | 30_6-6_internal_ | |
| | loop-symmetric_ | |
| | AGAAGA-ACAAAC | |
| 424 | -10_6-6_internal_ | GAUAUUGUGACAUAUCUCUGCACUCAU |
| | loop-symmetric_ | CACACAAAAGAUGCAAAAGAGGCAUA |
| | UUAGUU-CUUGCC | GGAUCCACAGGGAGGGGGCAUUCUAA |
| | 4_1-1_mismatch_ | UAUAUCUCUGCUUGCCUCAUC |
| | A-C | |
| | 30_6-6_internal_ | |
| | loop-symmetric_ | |
| | AGAAGA-AGAGGC | |
| 425 | -10_6-6_internal_ | GAUAUUGUGACAUAUCUCUGCACUCAU |
| | loop-symmetric_ | CACACAAAAGAUGCAAACACGGCAUAG |
| | UUAGUU-UCGGUU | GAUCCACAGGGAGGGGGCAUCUUAAU |
| | 5_1-1_mismatch_ | AUAUCUCUGUCGGUUUCAUC |
| | A-C | |
| | 30_6-6_internal_ | |
| | loop-symmetric_ | |
| | AGAAGA-CACGGC | |
| 426 | -10_6-6_internal_ | GAUAUUGUGACAUAUCUCUGCACUCAU |
| | loop-symmetric_ | CACACAAAAGAUGCAAAAUUAGAAUA |
| | UUAGUU-UUGCAU | GGAUCCACAGGGAGGGGGCAUUUUAA |
| | 30_6-6_internal_ | UAUAUCUCUGUUGCAUUCAUC |
| | loop-symmetric_ | |
| | AGAAGA-AUUAGA | |
| 427 | -10_6-6_internal_ | GAUAUUGUGACAUAUCUCUGCACUCAU |
| | loop-symmetric_ | CACACAAAAGAUGCAAUAAGCGUAUA |
| | UUAGUU-CCCAUC | GGAUCCACAGGGAGGGGGCAUUUUAA |
| | 0_1-1_mismatch_ | CAUAUCUCUGCCCAUCUCAUC |
| | A-C | |
| | 31_6-6_internal_ | |
| | loop-symmetric_ | |
| | GAAGAU-UAAGCG | |
| 428 | -10_6-6_internal_ | GAUAUUGUGACAUAUCUCUGCACUCAU |
| | loop-symmetric_ | CACACAAAAGAUGCAACAUACAUAUAG |
| | UUAGUU-CGUGAU | GAUCCACAGGGAGGGGGCAUUUCAAU |
| | 3_1-1_mismatch_ | AUAUCUCUGCGUGAUUCAUC |
| | A-C | |
| | 31_6-6_internal_ | |
| | loop-symmetric_ | |
| | GAAGAU-CAUACA | |
| 429 | -10_6-6_internal_ | GAUAUUGUGACAUAUCUCUGCACUCAU |
| | loop-symmetric_ | CACACAAAAGAUGCAAUGUAAGUAUA |
| | UUAGUU-UCAUCU | GGAUCCACAGGGAGGGGGCAUUCUAA |
| | 4_1-1_mismatch_ | UAUAUCUCUGUCAUCUUCAUC |
| | A-C | |
| | 31_6-6_internal_ | |
| | loop-symmetric_ | |
| | GAAGAU-UGUAAG | |
| 430 | -10_6-6_internal_ | GAUAUUGUGACAUAUCUCUGCACUCAU |
| | loop-symmetric_ | CACACAAAAGAUGCAACUAAAGUAUA |
| | UUAGUU-UAAAGC | GGAUCCACAGGGAGGGGGCAUCUUAA |
| | 5_1-1_mismatch_ | UAUAUCUCUGUAAAGCUCAUC |
| | A-C | |
| | 31_6-6_internal_ | |
| | loop-symmetric_ | |
| | GAAGAU-CUAAAG | |
| 431 | -10_6-6_internal_ | GAUAUUGUGACAUAUCUCUGCACUCAU |
| | loop-symmetric_ | CACACAAAAGAUGCAAUUGCUAUAUA |
| | UUAGUU-UUUACC | GGAUCCACAGGGAGGGGGCAUUUUAA |
| | 31_6-6_internal_ | UAUAUCUCUGUUUACCUCAUC |
| | loop-symmetric_ | |
| | GAAGAU-UUGCUA | |
| 432 | -10_6-6_internal_ | GAUAUUGUGACAUAUCUCUGCACUCAU |
| | loop-symmetric_ | CACACAAAAGAUGCACCAUAACUAUAG |
| | UUAGUU-UCAAGU | GAUCCACAGGGAGGGGGCAUUUUAAC |
| | 0_1-1_mismatch_ | AUAUCUCUGUCAAGUUCAUC |
| | A-C | |
| | 32_6-6_internal_ | |
| | loop-symmetric_ | |
| | AAGAUU-CCAUAA | |
| 433 | -10_6-6_internal_ | GAUAUUGUGACAUAUCUCUGCACUCAU |
| | loop-symmetric_ | CACACAAAAGAUGCAUCUAAGCUAUAG |
| | UUAGUU-CCCCCU | GAUCCACAGGGAGGGGGCAUUUCAAU |
| | 3_1-1_mismatch_ | AUAUCUCUGCCCCCUUCAUC |
| | A-C | |
| | 32_6-6_internal_ | |
| | loop-symmetric_ | |
| | AAGAUU-UCUAAG | |
| 434 | -10_6-6_internal_ | GAUAUUGUGACAUAUCUCUGCACUCAU |
| | loop-symmetric_ | CACACAAAAGAUGCAUCGAACCUAUAG |
| | UUAGUU-CCCCAU | GAUCCACAGGGAGGGGGCAUUCUAAU |
| | 4_1-1_mismatch_ | AUAUCUCUGCCCCAUUCAUC |
| | A-C | |
| | 32_6-6_internal_ | |
| | loop-symmetric_ | |
| | AAGAUU-UCGAAC | |
| 435 | -10_6-6_internal_ | GAUAUUGUGACAUAUCUCUGCACUCAU |
| | loop-symmetric_ | CACACAAAAGAUGCACGAAACCUAUAG |
| | UUAGUU-UUGGCC | GAUCCACAGGGAGGGGGCAUCUUAAU |
| | 5_1-1_mismatch_ | AUAUCUCUGUUGGCCUCAUC |
| | A-C | |
| | 32_6-6_internal_ | |
| | loop-symmetric_ | |
| | AAGAUU-CGAAAC | |
| 436 | -10_6-6_internal_ | GAUAUUGUGACAUAUCUCUGCACUCAU |
| | loop-symmetric_ | CACACAAAAGAUGCAUCAGGGCUAUAG |
| | UUAGUU-CUGGAU | GAUCCACAGGGAGGGGGCAUUUUAAU |
| | 32_6-6_internal_ | AUAUCUCUGCUGGAUUCAUC |
| | loop-symmetric_ | |
| | AAGAUU-UCAGGG | |
| 437 | -10_6-6_internal_ | GAUAUUGUGACAUAUCUCUGCACUCAU |
| | loop-symmetric_ | CACACAAAAGAUGCCAGAGGUCUAUAG |
| | UUAGUU-UUGAGU | GAUCCACAGGGAGGGGGCAUUUUAAC |
| | 0_1-1_mismatch_ | AUAUCUCUGUUGAGUUCAUC |
| | A-C | |
| | 33_6-6_internal_ | |
| | loop-symmetric_ | |
| | AGAUUU-CAGAGG | |
| 438 | -10_6-6_internal_ | GAUAUUGUGACAUAUCUCUGCACUCAU |
| | loop-symmetric_ | CACACAAAAGAUGCUUCGGCUCUAUAG |
| | UUAGUU-CGGGUC | GAUCCACAGGGAGGGGGCAUUUCAAU |
| | 3_1-1_mismatch_ | AUAUCUCUGCGGGUCUCAUC |
| | A-C | |
| | 33_6-6_internal_ | |
| | loop-symmetric_ | |
| | AGAUUU-UUCGGC | |
| 439 | -10_6-6_internal_ | GAUAUUGUGACAUAUCUCUGCACUCAU |
| | loop-symmetric_ | CACACAAAAGAUGCCCACUGUCUAUAG |
| | UUAGUU-CUGGUU | GAUCCACAGGGAGGGGGCAUUCUAAU |
| | 4_1-1_mismatch_ | AUAUCUCUGCUGGUUUCAUC |
| | A-C | |
| | 33_6-6_internal_ | |
| | loop-symmetric_ | |
| | AGAUUU-CCACUG | |
| 440 | -10_6-6_internal_ | GAUAUUGUGACAUAUCUCUGCACUCAU |
| | loop-symmetric_ | CACACAAAAGAUGCUUAACGUCUAUAG |
| | UUAGUU-UCAGUC | GAUCCACAGGGAGGGGGCAUCUUAAU |
| | 5_1-1_mismatch_ | AUAUCUCUGUCAGUCUCAUC |
| | A-C | |
| | 33_6-6_internal_ | |
| | loop-symmetric_ | |
| | AGAUUU-UUAACG | |
| 441 | -10_6-6_internal_ | GAUAUUGUGACAUAUCUCUGCACUCAU |
| | loop-symmetric_ | CACACAAAAGAUGCCCACAGUCUAUAG |
| | UUAGUU-CGAAUU | GAUCCACAGGGAGGGGGCAUUUUAAU |
| | 33_6-6_internal_ | AUAUCUCUGCGAAUUUCAUC |
| | loop-symmetric_ | |
| | AGAUUU-CCACAG | |
| 442 | -10_6-6_internal_ | GAUAUUGUGACAUAUCUCUGCACUCAU |
| | loop-symmetric_ | CACACAAAAGAUGGUUGUAUUCUAUA |
| | UUAGUU-UUUCUC | GGAUCCACAGGGAGGGGGCAUUUUAA |
| | 0_1-1_mismatch_ | CAUAUCUCUGUUUCUCUCAUC |
| | A-C | |
| | 34_6-6_internal_ | |
| | loop-symmetric_ | |
| | GAUUUG-GUUGUA | |
| 443 | -10_6-6_internal_ | GAUAUUGUGACAUAUCUCUGCACUCAU |
| | loop-symmetric_ | CACACAAAAGAUGACUGCGUUCUAUAG |
| | UUAGUU-UUACAC | GAUCCACAGGGAGGGGGCAUUUCAAU |
| | 3_1-1_mismatch_ | AUAUCUCUGUUACACUCAUC |
| | A-C | |
| | 34_6-6_internal_ | |
| | loop-symmetric_ | |
| | GAUUUG-ACUGCG | |
| 444 | -10_6-6_internal_ | GAUAUUGUGACAUAUCUCUGCACUCAU |
| | loop-symmetric_ | CACACAAAAGAUGGCCAGGUUCUAUAG |
| | UUAGUU-UGAUCC | GAUCCACAGGGAGGGGGCAUUCUAAU |
| | 4_1-1_mismatch_ | AUAUCUCUGUGAUCCUCAUC |
| | A-C | |
| | 34_6-6_internal_ | |
| | loop-symmetric_ | |
| | GAUUUG-GCCAGG | |
| 445 | -10_6-6_internal_ | GAUAUUGUGACAUAUCUCUGCACUCAU |
| | loop-symmetric_ | CACACAAAAGAUGAGUCAAUUCUAUA |
| | UUAGUU-CUUAUC | GGAUCCACAGGGAGGGGGCAUCUUAA |
| | 5_1-1_mismatch_ | UAUAUCUCUGCUUAUCUCAUC |
| | A-C | |
| | 34_6-6_internal_ | |
| | loop-symmetric_ | |
| | GAUUUG-AGUCAA | |
| 446 | -10_6-6_internal_ | GAUAUUGUGACAUAUCUCUGCACUCAU |
| | loop-symmetric_ | CACACAAAAGAUGGCGCAGUUCUAUAG |
| | UUAGUU-UCCAUC | GAUCCACAGGGAGGGGGCAUUUUAAU |
| | 34_6-6_internal_ | AUAUCUCUGUCCAUCUCAUC |
| | loop-symmetric_ | |
| | GAUUUG-GCGCAG | |
| 447 | -10_6-6_internal_ | GAUAUUGUGACAUAUCUCUGCACUCAU |
| | loop-symmetric_ | CACACAAAAGAUUAGUCACUUCUAUAG |
| | UUAGUU-UUGGUU | GAUCCACAGGGAGGGGGCAUUUUAAC |
| | 0_1-1_mismatch_ | AUAUCUCUGUUGGUUUCAUC |
| | A-C | |
| | 35_6-6_internal_ | |
| | loop-symmetric_ | |
| | AUUUGC-UAGUCA | |
| 448 | -10_6-6_internal_ | GAUAUUGUGACAUAUCUCUGCACUCAU |
| | loop-symmetric_ | CACACAAAAGAUAUGUUCCUUCUAUAG |
| | UUAGUU-UCAAAU | GAUCCACAGGGAGGGGGCAUUUCAAU |
| | 3_1-1_mismatch_ | AUAUCUCUGUCAAAUUCAUC |
| | A-C | |
| | 35_6-6_internal_ | |
| | loop-symmetric_ | |
| | AUUUGC-AUGUUC | |
| 449 | -10_6-6_internal_ | GAUAUUGUGACAUAUCUCUGCACUCAU |
| | loop-symmetric_ | CACACAAAAGAUUAACGGCUUCUAUAG |
| | UUAGUU-CGGAUC | GAUCCACAGGGAGGGGGCAUUCUAAU |
| | 4_1-1_mismatch_ | AUAUCUCUGCGGAUCUCAUC |
| | A-C | |
| | 35_6-6_internal_ | |
| | loop-symmetric_ | |
| | AUUUGC-UAACGG | |
| 450 | -10_6-6_internal_ | GAUAUUGUGACAUAUCUCUGCACUCAU |
| | loop-symmetric_ | CACACAAAAGAUUUUCACCUUCUAUAG |
| | UUAGUU-UAAGGU | GAUCCACAGGGAGGGGGCAUCUUAAU |
| | 5_1-1_mismatch_ | AUAUCUCUGUAAGGUUCAUC |
| | A-C | |
| | 35_6-6_internal_ | |
| | loop-symmetric_ | |
| | AUUUGC-UUUCAC | |
| 451 | -10_6-6_internal_ | GAUAUUGUGACAUAUCUCUGCACUCAU |
| | loop-symmetric_ | CACACAAAAGAUAAAUAGCUUCUAUA |
| | UUAGUU-UUUAUU | GGAUCCACAGGGAGGGGGCAUUUUAA |
| | 35_6-6_internal_ | UAUAUCUCUGUUUAUUUCAUC |
| | loop-symmetric_ | |
| | AUUUGC-AAAUAG | |
| 452 | -10_6-6_internal_ | GAUAUUGUGACAUAUCUCUGCACUCAU |
| | loop-symmetric_ | CACACAAAAGAAUUUAUUCUUCUAUA |
| | UUAGUU-CCCACC | GGAUCCACAGGGAGGGGGCAUUUUAA |
| | 0_1-1_mismatch_ | CAUAUCUCUGCCCACCUCAUC |
| | A-C | |
| | 36_6-6_internal_ | |
| | loop-symmetric_ | |
| | UUUGCA-AUUUAU | |
| 453 | -10_6-6_internal_ | GAUAUUGUGACAUAUCUCUGCACUCAU |
| | loop-symmetric_ | CACACAAAAGAGUCCGCUCUUCUAUAG |
| | UUAGUU-UUGGUC | GAUCCACAGGGAGGGGGCAUUUCAAU |
| | 3_1-1_mismatch_ | AUAUCUCUGUUGGUCUCAUC |
| | A-C | |
| | 36_6-6_internal_ | |
| | loop-symmetric_ | |
| | UUUGCA-GUCCGC | |
| 454 | -10_6-6_internal_ | GAUAUUGUGACAUAUCUCUGCACUCAU |
| | loop-symmetric_ | CACACAAAAGAACAUGUUCUUCUAUAG |
| | UUAGUU-UAUAUC | GAUCCACAGGGAGGGGGCAUUCUAAU |
| | 4_1-1_mismatch_ | AUAUCUCUGUAUAUCUCAUC |
| | A-C | |
| | 36_6-6_internal_ | |
| | loop-symmetric_ | |
| | UUUGCA-ACAUGU | |
| 455 | -10_6-6_internal_ | GAUAUUGUGACAUAUCUCUGCACUCAU |
| | loop-symmetric_ | CACACAAAAGACCCCUUUCUUCUAUAG |
| | UUAGUU-CGAAGU | GAUCCACAGGGAGGGGGCAUCUUAAU |
| | 5_1-1_mismatch_ | AUAUCUCUGCGAAGUUCAUC |
| | A-C | |
| | 36_6-6_internal_ | |
| | loop-symmetric_ | |
| | UUUGCA-CCCCUU | |
| 456 | -10_6-6_internal_ | GAUAUUGUGACAUAUCUCUGCACUCAU |
| | loop-symmetric_ | CACACAAAAGAAAGUCUUCUUCUAUAG |
| | UUAGUU-UAGGGC | GAUCCACAGGGAGGGGGCAUUUUAAU |
| | 36_6-6_internal_ | AUAUCUCUGUAGGGCUCAUC |
| | loop-symmetric_ | |
| | UUUGCA-AAGUCU | |
| 457 | -10_6-6_internal_ | GAUAUUGUGACAUAUCUCUGCACUCAU |
| | loop-symmetric_ | CACACAAAAGUAUAGUAUCUUCUAUA |
| | UUAGUU-UUGCCC | GGAUCCACAGGGAGGGGGCAUUUUAA |
| | 0_1-1_mismatch_ | CAUAUCUCUGUUGCCCUCAUC |
| | A-C | |
| | 37_6-6_internal_ | |
| | loop-symmetric_ | |
| | UUGCAU-UAUAGU | |
| 458 | -10_6-6_internal_ | GAUAUUGUGACAUAUCUCUGCACUCAU |
| | loop-symmetric_ | CACACAAAAGUACUACAUCUUCUAUAG |
| | UUAGUU-UGUGUU | GAUCCACAGGGAGGGGGCAUUUCAAU |
| | 3_1-1_mismatch_ | AUAUCUCUGUGUGUUUCAUC |
| | A-C | |
| | 37_6-6_internal_ | |
| | loop-symmetric_ | |
| | UUGCAU-UACUAC | |
| 459 | -10_6-6_internal_ | GAUAUUGUGACAUAUCUCUGCACUCAU |
| | loop-symmetric_ | CACACAAAAGCGAUUCAUCUUCUAUAG |
| | UUAGUU-CCCCCU | GAUCCACAGGGAGGGGGCAUUCUAAU |
| | 4_1-1_mismatch_ | AUAUCUCUGCCCCCUUCAUC |
| | A-C | |
| | 37_6-6_internal_ | |
| | loop-symmetric_ | |
| | UUGCAU-CGAUUC | |
| 460 | -10_6-6_internal_ | GAUAUUGUGACAUAUCUCUGCACUCAU |
| | loop-symmetric_ | CACACAAAAGCAAAUCAUCUUCUAUAG |
| | UUAGUU-CCAAAC | GAUCCACAGGGAGGGGGCAUCUUAAU |
| | 5_1-1_mismatch_ | AUAUCUCUGCCAAACUCAUC |
| | A-C | |
| | 37_6-6_internal_ | |
| | loop-symmetric_ | |
| | UUGCAU-CAAAUC | |
| 461 | -10_6-6_internal_ | GAUAUUGUGACAUAUCUCUGCACUCAU |
| | loop-symmetric_ | CACACAAAAGUAAAAUAUCUUCUAUA |
| | UUAGUU-UUAUCC | GGAUCCACAGGGAGGGGGCAUUUUAA |
| | 37_6-6_internal_ | UAUAUCUCUGUUAUCCUCAUC |
| | loop-symmetric_ | |
| | UUGCAU-UAAAAU | |
| 462 | -10_6-6_internal_ | GAUAUUGUGACAUAUCUCUGCACUCAU |
| | loop-symmetric_ | CACACAAAACUCCACAAUCUUCUAUAG |
| | UUAGUU-UAUCUC | GAUCCACAGGGAGGGGGCAUUUUAAC |
| | 0_1-1_mismatch_ | AUAUCUCUGUAUCUCUCAUC |
| | A-C | |
| | 38_6-6_internal_ | |
| | loop-symmetric_ | |
| | UGCAUC-CUCCAC | |
| 463 | -10_6-6_internal_ | GAUAUUGUGACAUAUCUCUGCACUCAU |
| | loop-symmetric_ | CACACAAAACUAACUAAUCUUCUAUAG |
| | UUAGUU-UUGCAU | GAUCCACAGGGAGGGGGCAUUUCAAU |
| | 3_1-1_mismatch_ | AUAUCUCUGUUGCAUUCAUC |
| | A-C | |
| | 38_6-6_internal_ | |
| | loop-symmetric_ | |
| | UGCAUC-CUAACU | |
| 464 | -10_6-6_internal_ | GAUAUUGUGACAUAUCUCUGCACUCAU |
| | loop-symmetric_ | CACACAAAACCACUUAAUCUUCUAUAG |
| | UUAGUU-CCAGUU | GAUCCACAGGGAGGGGGCAUUCUAAU |
| | 4_1-1_mismatch_ | AUAUCUCUGCCAGUUUCAUC |
| | A-C | |
| | 38_6-6_internal_ | |
| | loop-symmetric_ | |
| | UGCAUC-CCACUU | |
| 465 | -10_6-6_internal_ | GAUAUUGUGACAUAUCUCUGCACUCAU |
| | loop-symmetric_ | CACACAAAAUACUAUAAUCUUCUAUAG |
| | UUAGUU-UUGUCU | GAUCCACAGGGAGGGGGCAUCUUAAU |
| | 5_1-1_mismatch_ | AUAUCUCUGUUGUCUUCAUC |
| | A-C | |
| | 38_6-6_internal_ | |
| | loop-symmetric_ | |
| | UGCAUC-UACUAU | |
| 466 | -10_6-6_internal_ | GAUAUUGUGACAUAUCUCUGCACUCAU |
| | loop-symmetric_ | CACACAAAAAUCAACAAUCUUCUAUAG |
| | UUAGUU-UCUUCC | GAUCCACAGGGAGGGGGCAUUUUAAU |
| | 38_6-6_internal_ | AUAUCUCUGUCUUCCUCAUC |
| | loop-symmetric_ | |
| | UGCAUC-AUCAAC | |
| 467 | -10_6-6_internal_ | GAUAUUGUGACAUAUCUCUGCACUCAU |
| | loop-symmetric_ | CACACAAACUAGUAAAAUCUUCUAUAG |
| | UUAGUU-CCGUCU | GAUCCACAGGGAGGGGGCAUUUUAAC |
| | 0_1-1_mismatch_ | AUAUCUCUGCCGUCUUCAUC |
| | A-C | |
| | 39_6-6_internal_ | |
| | loop-symmetric_ | |
| | GCAUCU-CUAGUA | |
| 468 | -10_6-6_internal_ | GAUAUUGUGACAUAUCUCUGCACUCAU |
| | loop-symmetric_ | CACACAAACUUCAGAAAUCUUCUAUAG |
| | UUAGUU-UUUCUC | GAUCCACAGGGAGGGGGCAUUUCAAU |
| | 3_1-1_mismatch_ | AUAUCUCUGUUUCUCUCAUC |
| | A-C | |
| | 39_6-6_internal_ | |
| | loop-symmetric_ | |
| | GCAUCU-CUUCAG | |
| 469 | -10_6-6_internal_ | GAUAUUGUGACAUAUCUCUGCACUCAU |
| | loop-symmetric_ | CACACAAAUUUGGAAAAUCUUCUAUA |
| | UUAGUU-CUUAGU | GGAUCCACAGGGAGGGGGCAUUCUAA |
| | 4_1-1_mismatch_ | UAUAUCUCUGCUUAGUUCAUC |
| | A-C | |
| | 39_6-6_internal_ | |
| | loop-symmetric_ | |
| | GCAUCU-UUUGGA | |
| 470 | -10_6-6_internal_ | GAUAUUGUGACAUAUCUCUGCACUCAU |
| | loop-symmetric_ | CACACAAACACCAAAAAUCUUCUAUAG |
| | UUAGUU-UUUGUC | GAUCCACAGGGAGGGGGCAUCUUAAU |
| | 5_1-1_mismatch_ | AUAUCUCUGUUUGUCUCAUC |
| | A-C | |
| | 39_6-6_internal_ | |
| | loop-symmetric_ | |
| | GCAUCU-CACCAA | |
| 471 | -10_6-6_internal_ | GAUAUUGUGACAUAUCUCUGCACUCAU |
| | loop-symmetric_ | CACACAAAUUCUUAAAAUCUUCUAUAG |
| | UUAGUU-CCAGUC | GAUCCACAGGGAGGGGGCAUUUUAAU |
| | 39_6-6_internal_ | AUAUCUCUGCCAGUCUCAUC |
| | loop-symmetric_ | |
| | GCAUCU-UUCUUA | |
| 472 | -10_6-6_internal_ | GAUAUUGUGACAUAUCUCUGCACUCAU |
| | loop-symmetric_ | CACACAACCUACACAAAUCUUCUAUAG |
| | UUAGUU-UUGCAC | GAUCCACAGGGAGGGGGCAUUUUAAC |
| | 0_1-1_mismatch_ | AUAUCUCUGUUGCACUCAUC |
| | A-C | |
| | 40_6-6_internal_ | |
| | loop-symmetric_ | |
| | CAUCUU-CCUACA | |
| 473 | -10_6-6_internal_ | GAUAUUGUGACAUAUCUCUGCACUCAU |
| | loop-symmetric_ | CACACAACGAAAUCAAAUCUUCUAUAG |
| | UUAGUU-CCACCU | GAUCCACAGGGAGGGGGCAUUUCAAU |
| | 3_1-1_mismatch_ | AUAUCUCUGCCACCUUCAUC |
| | A-C | |
| | 40_6-6_internal_ | |
| | loop-symmetric_ | |
| | CAUCUU-CGAAAU | |
| 474 | -10_6-6_internal_ | GAUAUUGUGACAUAUCUCUGCACUCAU |
| | loop-symmetric_ | CACACAAUACACUCAAAUCUUCUAUAG |
| | UUAGUU-CCAAUC | GAUCCACAGGGAGGGGGCAUUCUAAU |
| | 4_1-1_mismatch_ | AUAUCUCUGCCAAUCUCAUC |
| | A-C | |
| | 40_6-6_internal_ | |
| | loop-symmetric_ | |
| | CAUCUU-UACACU | |
| 475 | -10_6-6_internal_ | GAUAUUGUGACAUAUCUCUGCACUCAU |
| | loop-symmetric_ | CACACAAUUUAGCCAAAUCUUCUAUAG |
| | UUAGUU-CAGGAC | GAUCCACAGGGAGGGGGCAUCUUAAU |
| | 5_1-1_mismatch_ | AUAUCUCUGCAGGACUCAUC |
| | A-C | |
| | 40_6-6_internal_ | |
| | loop-symmetric_ | |
| | CAUCUU-UUUAGC | |
| 476 | -10_6-6_internal_ | GAUAUUGUGACAUAUCUCUGCACUCAU |
| | loop-symmetric_ | CACACAACGAAACCAAAUCUUCUAUAG |
| | UUAGUU-UUUACC | GAUCCACAGGGAGGGGGCAUUUUAAU |
| | 40_6-6_internal_ | AUAUCUCUGUUUACCUCAUC |
| | loop-symmetric_ | |
| | CAUCUU-CGAAAC | |
| 477 | -10_6-6_internal_ | GAUAUUGUGACAUAUCUCUGCACUCAU |
| | loop-symmetric_ | CACACAUUUCCAGCAAAUCUUCUAUAG |
| | UUAGUU-CUUAUU | GAUCCACAGGGAGGGGGCAUUUUAAC |
| | 0_1-1_mismatch_ | AUAUCUCUGCUUAUUUCAUC |
| | A-C | |
| | 41_6-6_internal_ | |
| | loop-symmetric_ | |
| | AUCUUU-UUUCCA | |
| 478 | -10_6-6_internal_ | GAUAUUGUGACAUAUCUCUGCACUCAU |
| | loop-symmetric_ | CACACACACGUGGCAAAUCUUCUAUAG |
| | UUAGUU-CGGGGU | GAUCCACAGGGAGGGGGCAUUUCAAU |
| | 3_1-1_mismatch_ | AUAUCUCUGCGGGGUUCAUC |
| | A-C | |
| | 41_6-6_internal_ | |
| | loop-symmetric_ | |
| | AUCUUU-CACGUG | |
| 479 | -10_6-6_internal_ | GAUAUUGUGACAUAUCUCUGCACUCAU |
| | loop-symmetric_ | CACACACCACACGCAAAUCUUCUAUAG |
| | UUAGUU-UUUUCC | GAUCCACAGGGAGGGGGCAUUCUAAU |
| | 4_1-1_mismatch_ | AUAUCUCUGUUUUCCUCAUC |
| | A-C | |
| | 41_6-6_internal_ | |
| | loop-symmetric_ | |
| | AUCUUU-CCACAC | |
| 480 | -10_6-6_internal_ | GAUAUUGUGACAUAUCUCUGCACUCAU |
| | loop-symmetric_ | CACACACCUUUCGCAAAUCUUCUAUAG |
| | UUAGUU-UCAUCU | GAUCCACAGGGAGGGGGCAUCUUAAU |
| | 5_1-1_mismatch_ | AUAUCUCUGUCAUCUUCAUC |
| | A-C | |
| | 41_6-6_internal_ | |
| | loop-symmetric_ | |
| | AUCUUU-CCUUUC | |
| 481 | -10_6-6_internal_ | GAUAUUGUGACAUAUCUCUGCACUCAU |
| | loop-symmetric_ | CACACAUCGCCGGCAAAUCUUCUAUAG |
| | UUAGUU-CCCCCC | GAUCCACAGGGAGGGGGCAUUUUAAU |
| | 41_6-6_internal_ | AUAUCUCUGCCCCCCUCAUC |
| | loop-symmetric_ | |
| | AUCUUU-UCGCCG | |
| 482 | -10_6-6_internal_ | GAUAUUGUGACAUAUCUCUGCACUCAU |
| | loop-symmetric_ | CACACUACGCCUGCAAAUCUUCUAUAG |
| | UUAGUU-UUACAU | GAUCCACAGGGAGGGGGCAUUUUAAC |
| | 0_1-1_mismatch_ | AUAUCUCUGUUACAUUCAUC |
| | A-C | |
| | 42_6-6_internal_ | |
| | loop-symmetric_ | |
| | UCUUUU-UACGCC | |
| 483 | -10_6-6_internal_ | GAUAUUGUGACAUAUCUCUGCACUCAU |
| | loop-symmetric_ | CACACCCUGUCUGCAAAUCUUCUAUAG |
| | UUAGUU-UCCACU | GAUCCACAGGGAGGGGGCAUUUCAAU |
| | 3_1-1_mismatch_ | AUAUCUCUGUCCACUUCAUC |
| | A-C | |
| | 42_6-6_internal_ | |
| | loop-symmetric_ | |
| | UCUUUU-CCUGUC | |
| 484 | -10_6-6_internal_ | GAUAUUGUGACAUAUCUCUGCACUCAU |
| | loop-symmetric_ | CACACUCCAUUUGCAAAUCUUCUAUAG |
| | UUAGUU-UCUCGC | GAUCCACAGGGAGGGGGCAUUCUAAU |
| | 4_1-1_mismatch_ | AUAUCUCUGUCUCGCUCAUC |
| | A-C | |
| | 42_6-6_internal_ | |
| | loop-symmetric_ | |
| | UCUUUU-UCCAUU | |
| 485 | -10_6-6_internal_ | GAUAUUGUGACAUAUCUCUGCACUCAU |
| | loop-symmetric_ | CACACUCCAUCUGCAAAUCUUCUAUAG |
| | UUAGUU-UGUAUU | GAUCCACAGGGAGGGGGCAUCUUAAU |
| | 5_1-1_mismatch_ | AUAUCUCUGUGUAUUUCAUC |
| | A-C | |
| | 42_6-6_internal_ | |
| | loop-symmetric_ | |
| | UCUUUU-UCCAUC | |
| 486 | -10_6-6_internal_ | GAUAUUGUGACAUAUCUCUGCACUCAU |
| | loop-symmetric_ | CACACUGUCCCUGCAAAUCUUCUAUAG |
| | UUAGUU-CCAGUC | GAUCCACAGGGAGGGGGCAUUUUAAU |
| | 42_6-6_internal_ | AUAUCUCUGCCAGUCUCAUC |
| | loop-symmetric_ | |
| | UCUUUU-UGUCCC | |
| 487 | -10_6-6_internal_ | GAUAUUGUGACAUAUCUCUGCACUCAU |
| | loop-symmetric_ | CACAAUCGCUAUGCAAAUCUUCUAUAG |
| | UUAGUU-CCUCAC | GAUCCACAGGGAGGGGGCAUUUUAAC |
| | 0_1-1_mismatch_ | AUAUCUCUGCCUCACUCAUC |
| | A-C | |
| | 43_6-6_internal_ | |
| | loop-symmetric_ | |
| | CUUUUG-AUCGCU | |
| 488 | -10_6-6_internal_ | GAUAUUGUGACAUAUCUCUGCACUCAU |
| | loop-symmetric_ | CACAAGCCAAAUGCAAAUCUUCUAUAG |
| | UUAGUU-CCGUCC | GAUCCACAGGGAGGGGGCAUUUCAAU |
| | 3_1-1_mismatch_ | AUAUCUCUGCCGUCCUCAUC |
| | A-C | |
| | 43_6-6_internal_ | |
| | loop-symmetric_ | |
| | CUUUUG-AGCCAA | |
| 489 | -10_6-6_internal_ | GAUAUUGUGACAUAUCUCUGCACUCAU |
| | loop-symmetric_ | CACAGUUCUCAUGCAAAUCUUCUAUAG |
| | UUAGUU-UUUCUU | GAUCCACAGGGAGGGGGCAUUCUAAU |
| | 4_1-1_mismatch_ | AUAUCUCUGUUUCUUUCAUC |
| | A-C | |
| | 43_6-6_internal_ | |
| | loop-symmetric_ | |
| | CUUUUG-GUUCUC | |
| 490 | -10_6-6_internal_ | GAUAUUGUGACAUAUCUCUGCACUCAU |
| | loop-symmetric_ | CACAGCUCAUAUGCAAAUCUUCUAUAG |
| | UUAGUU-CUUCUC | GAUCCACAGGGAGGGGGCAUCUUAAU |
| | 5_1-1_mismatch_ | AUAUCUCUGCUUCUCUCAUC |
| | A-C | |
| | 43_6-6_internal_ | |
| | loop-symmetric_ | |
| | CUUUUG-GCUCAU | |
| 491 | -10_6-6_internal_ | GAUAUUGUGACAUAUCUCUGCACUCAU |
| | loop-symmetric_ | CACAAAGUUUAUGCAAAUCUUCUAUA |
| | UUAGUU-UCGAGU | GGAUCCACAGGGAGGGGGCAUUUUAA |
| | 43_6-6_internal_ | UAUAUCUCUGUCGAGUUCAUC |
| | loop-symmetric_ | |
| | CUUUUG-AAGUUU | |
| 492 | -10_6-6_internal_ | GAUAUUGUGACAUAUCUCUGCACUCAU |
| | loop-symmetric_ | CACUGGCAUGAUGCAAAUCUUCUAUAG |
| | UUAGUU-CGAGGU | GAUCCACAGGGAGGGGGCAUUUUAAC |
| | 0_1-1_mismatch_ | AUAUCUCUGCGAGGUUCAUC |
| | A-C | |
| | 44_6-6_internal_ | |
| | loop-symmetric_ | |
| | UUUUGU-UGGCAU | |
| 493 | -10_6-6_internal_ | GAUAUUGUGACAUAUCUCUGCACUCAU |
| | loop-symmetric_ | CACUGUCUUGAUGCAAAUCUUCUAUAG |
| | UUAGUU-CCUCUU | GAUCCACAGGGAGGGGGCAUUUCAAU |
| | 3_1-1_mismatch_ | AUAUCUCUGCCUCUUUCAUC |
| | A-C | |
| | 44_6-6_internal_ | |
| | loop-symmetric_ | |
| | UUUUGU-UGUCUU | |
| 494 | -10_6-6_internal_ | GAUAUUGUGACAUAUCUCUGCACUCAU |
| | loop-symmetric_ | CACCCCUCCGAUGCAAAUCUUCUAUAG |
| | UUAGUU-CGUACC | GAUCCACAGGGAGGGGGCAUUCUAAU |
| | 4_1-1_mismatch_ | AUAUCUCUGCGUACCUCAUC |
| | A-C | |
| | 44_6-6_internal_ | |
| | loop-symmetric_ | |
| | UUUUGU-CCCUCC | |
| 495 | -10_6-6_internal_ | GAUAUUGUGACAUAUCUCUGCACUCAU |
| | loop-symmetric_ | CACUGCCUCGAUGCAAAUCUUCUAUAG |
| | UUAGUU-UUGGUU | GAUCCACAGGGAGGGGGCAUCUUAAU |
| | 5_1-1_mismatch_ | AUAUCUCUGUUGGUUUCAUC |
| | A-C | |
| | 44_6-6_internal_ | |
| | loop-symmetric_ | |
| | UUUUGU-UGCCUC | |
| 496 | -10_6-6_internal_ | GAUAUUGUGACAUAUCUCUGCACUCAU |
| | loop-symmetric_ | CACCUUGCCGAUGCAAAUCUUCUAUAG |
| | UUAGUU-CGGAUC | GAUCCACAGGGAGGGGGCAUUUUAAU |
| | 44_6-6_internal_ | AUAUCUCUGCGGAUCUCAUC |
| | loop-symmetric_ | |
| | UUUUGU-CUUGCC | |
| 497 | -9_6-6_internal_ | GAUAUUGUGACAUAUCUCUGCACUCAU |
| | loop-symmetric_ | CACACAAAAGAUGCAAAUCUUCUAUAG |
| | UAGUUC-AUUUAU | GAUCCACAGGGAGGGGGCAUUUUAAC |
| | 0_1-1_mismatch_ | AUAUCUCUAUUUAUAUCAUC |
| | A-C | |
| 498 | -9_6-6_internal_ | GAUAUUGUGACAUAUCUCUGCACUCAU |
| | loop-symmetric_ | CACACAAAAGAUGCAAAUCUUCUAUAG |
| | UAGUUC-UUGUAC | GAUCCACAGGGAGGGGGCAUUUCAAU |
| | 3_1-1_mismatch_ | AUAUCUCUUUGUACAUCAUC |
| | A-C | |
| 499 | -9_6-6_internal_ | GAUAUUGUGACAUAUCUCUGCACUCAU |
| | loop-symmetric_ | CACACAAAAGAUGCAAAUCUUCUAUAG |
| | UAGUUC-CAUUUU | GAUCCACAGGGAGGGGGCAUUCUAAU |
| | 4_1-1_mismatch_ | AUAUCUCUCAUUUUAUCAUC |
| | A-C | |
| 500 | -9_6-6_internal_ | GAUAUUGUGACAUAUCUCUGCACUCAU |
| | loop-symmetric_ | CACACAAAAGAUGCAAAUCUUCUAUAG |
| | UAGUUC-CUCCAC | GAUCCACAGGGAGGGGGCAUCUUAAU |
| | 5_1-1_mismatch_ | AUAUCUCUCUCCACAUCAUC |
| | A-C | |
| 501 | -9_6-6_internal_ | GAUAUUGUGACAUAUCUCUGCACUCAU |
| | loop-symmetric_ | CACACAAAAGAUGCAAAUCUUCUAUAG |
| | UAGUUC-UCAAAU | GAUCCACAGGGAGGGGGCAUUUUAAU |
| | | AUAUCUCUUCAAAUAUCAUC |
| 502 | -9_6-6_internal_ | GAUAUUGUGACAUAUCUCUGCACUCAU |
| | loop-symmetric_ | CACACAAAAGAUGCAAAUCUUCUAUAG |
| | UAGUUC-UUUUGC | ACUAUUCAGGGAGGGGGCAUUUUAAC |
| | 0_1-1_mismatch_ | AUAUCUCUUUUUGCAUCAUC |
| | A-C | |
| | 20_6-6_internal_ | |
| | loop-symmetric_ | |
| | UGGAUC-ACUAUU | |
| 503 | -9_6-6_internal_ | GAUAUUGUGACAUAUCUCUGCACUCAU |
| | loop-symmetric_ | CACACAAAAGAUGCAAAUCUUCUAUAG |
| | UAGUUC-UCCGAU | CGCUUUCAGGGAGGGGGCAUUUCAAU |
| | 3_1-1_mismatch_ | AUAUCUCUUCCGAUAUCAUC |
| | A-C | |
| | 20_6-6_internal_ | |
| | loop-symmetric_ | |
| | UGGAUC-CGCUUU | |
| 504 | -9_6-6_internal_ | GAUAUUGUGACAUAUCUCUGCACUCAU |
| | loop-symmetric_ | CACACAAAAGAUGCAAAUCUUCUAUAG |
| | UAGUUC-AUGUAU | AAGGAUCAGGGAGGGGGCAUUCUAAU |
| | 4_1-1_mismatch_ | AUAUCUCUAUGUAUAUCAUC |
| | A-C | |
| | 20_6-6_internal_ | |
| | loop-symmetric_ | |
| | UGGAUC-AAGGAU | |
| 505 | -9_6-6_internal_ | GAUAUUGUGACAUAUCUCUGCACUCAU |
| | loop-symmetric_ | CACACAAAAGAUGCAAAUCUUCUAUAG |
| | UAGUUC-ACGUCC | UCGGGUCAGGGAGGGGGCAUCUUAAU |
| | 5_1-1_mismatch_ | AUAUCUCUACGUCCAUCAUC |
| | A-C | |
| | 20_6-6_internal_ | |
| | loop-symmetric_ | |
| | UGGAUC-UCGGGU | |
| 506 | -9_6-6_internal_ | GAUAUUGUGACAUAUCUCUGCACUCAU |
| | loop-symmetric_ | CACACAAAAGAUGCAAAUCUUCUAUAG |
| | UAGUUC-UUCACC | UACUUUCAGGGAGGGGGCAUUUUAAU |
| | 20_6-6_internal_ | AUAUCUCUUUCACCAUCAUC |
| | loop-symmetric_ | |
| | UGGAUC-UACUUU | |
| 507 | -9_6-6_internal_ | GAUAUUGUGACAUAUCUCUGCACUCAU |
| | loop-symmetric_ | CACACAAAAGAUGCAAAUCUUCUAUAU |
| | UAGUUC-UAUGGU | AGAAAACAGGGAGGGGGCAUUUUAAC |
| | 0_1-1_mismatch_ | AUAUCUCUUAUGGUAUCAUC |
| | A-C | |
| | 21_6-6_internal_ | |
| | loop-symmetric_ | |
| | GGAUCC-UAGAAA | |
| 508 | -9_6-6_internal_ | GAUAUUGUGACAUAUCUCUGCACUCAU |
| | loop-symmetric_ | CACACAAAAGAUGCAAAUCUUCUAUAU |
| | UAGUUC-UUUUCC | CUACAACAGGGAGGGGGCAUUUCAAU |
| | 3_1-1_mismatch_ | AUAUCUCUUUUUCCAUCAUC |
| | A-C | |
| | 21_6-6_internal_ | |
| | loop-symmetric_ | |
| | GGAUCC-UCUACA | |
| 509 | -9_6-6_internal_ | GAUAUUGUGACAUAUCUCUGCACUCAU |
| | loop-symmetric_ | CACACAAAAGAUGCAAAUCUUCUAUAA |
| | UAGUUC-UUCCGC | CCUAAACAGGGAGGGGGCAUUCUAAU |
| | 4_1-1_mismatch_ | AUAUCUCUUUCCGCAUCAUC |
| | A-C | |
| | 21_6-6_internal_ | |
| | loop-symmetric_ | |
| | GGAUCC-ACCUAA | |
| 510 | -9_6-6_internal_ | GAUAUUGUGACAUAUCUCUGCACUCAU |
| | loop-symmetric_ | CACACAAAAGAUGCAAAUCUUCUAUAA |
| | UAGUUC-UCCCGC | CGCAGACAGGGAGGGGGCAUCUUAAU |
| | 5_1-1_mismatch_ | AUAUCUCUUCCCGCAUCAUC |
| | A-C | |
| | 21_6-6_internal_ | |
| | loop-symmetric_ | |
| | GGAUCC-ACGCAG | |
| 511 | -9_6-6_internal_ | GAUAUUGUGACAUAUCUCUGCACUCAU |
| | loop-symmetric_ | CACACAAAAGAUGCAAAUCUUCUAUAC |
| | UAGUUC-AGUGAU | UUAGAACAGGGAGGGGGCAUUUUAAU |
| | 21_6-6_internal_ | AUAUCUCUAGUGAUAUCAUC |
| | loop-symmetric_ | |
| | GGAUCC-CUUAGA | |
| 512 | -9_6-6_internal_ | GAUAUUGUGACAUAUCUCUGCACUCAU |
| | loop-symmetric_ | CACACAAAAGAUGCAAAUCUUCUAUUC |
| | UAGUUC-AUCAUU | UCCGCACAGGGAGGGGGCAUUUUAACA |
| | 0_1-1_mismatch_ | UAUCUCUAUCAUUAUCAUC |
| | A-C | |
| | 22_6-6_internal_ | |
| | loop-symmetric_ | |
| | GAUCCU-UCUCCG | |
| 513 | -9_6-6_internal_ | GAUAUUGUGACAUAUCUCUGCACUCAU |
| | loop-symmetric_ | CACACAAAAGAUGCAAAUCUUCUAUCU |
| | UAGUUC-UAUGAU | ACCACACAGGGAGGGGGCAUUUCAAUA |
| | 3_1-1_mismatch_ | UAUCUCUUAUGAUAUCAUC |
| | A-C | |
| | 22_6-6_internal_ | |
| | loop-symmetric_ | |
| | GAUCCU-CUACCA | |
| 514 | -9_6-6_internal_ | GAUAUUGUGACAUAUCUCUGCACUCAU |
| | loop-symmetric_ | CACACAAAAGAUGCAAAUCUUCUAUUA |
| | UAGUUC-UCCUCC | AUUACACAGGGAGGGGGCAUUCUAAU |
| | 4_1-1_mismatch_ | AUAUCUCUUCCUCCAUCAUC |
| | A-C | |
| | 22_6-6_internal_ | |
| | loop-symmetric_ | |
| | GAUCCU-UAAUUA | |
| 515 | -9_6-6_internal_ | GAUAUUGUGACAUAUCUCUGCACUCAU |
| | loop-symmetric_ | CACACAAAAGAUGCAAAUCUUCUAUUA |
| | UAGUUC-AUGGGU | UGUACACAGGGAGGGGGCAUCUUAAU |
| | 5_1-1_mismatch_ | AUAUCUCUAUGGGUAUCAUC |
| | A-C | |
| | 22_6-6_internal_ | |
| | loop-symmetric_ | |
| | GAUCCU-UAUGUA | |
| 516 | -9_6-6_internal_ | GAUAUUGUGACAUAUCUCUGCACUCAU |
| | loop-symmetric_ | CACACAAAAGAUGCAAAUCUUCUAUUU |
| | UAGUUC-CAGUCC | CGCGCACAGGGAGGGGGCAUUUUAAU |
| | 22_6-6_internal_ | AUAUCUCUCAGUCCAUCAUC |
| | loop-symmetric_ | |
| | GAUCCU-UUCGCG | |
| 517 | -9_6-6_internal_ | GAUAUUGUGACAUAUCUCUGCACUCAU |
| | loop-symmetric_ | CACACAAAAGAUGCAAAUCUUCUAAUU |
| | UAGUUC-CUCCAU | GCACCACAGGGAGGGGGCAUUUUAACA |
| | 0_1-1_mismatch_ | UAUCUCUCUCCAUAUCAUC |
| | A-C | |
| | 23_6-6_internal_ | |
| | loop-symmetric_ | |
| | AUCCUA-AUUGCA | |
| 518 | -9_6-6_internal_ | GAUAUUGUGACAUAUCUCUGCACUCAU |
| | loop-symmetric_ | CACACAAAAGAUGCAAAUCUUCUAGAU |
| | UAGUUC-CAUGCC | UAACCACAGGGAGGGGGCAUUUCAAU |
| | 3_1-1_mismatch_ | AUAUCUCUCAUGCCAUCAUC |
| | A-C | |
| | 23_6-6_internal_ | |
| | loop-symmetric_ | |
| | AUCCUA-GAUUAA | |
| 519 | -9_6-6_internal_ | GAUAUUGUGACAUAUCUCUGCACUCAU |
| | loop-symmetric_ | CACACAAAAGAUGCAAAUCUUCUAGUC |
| | UAGUUC-UCCUCC | CUACCACAGGGAGGGGGCAUUCUAAUA |
| | 4_1-1_mismatch_ | UAUCUCUUCCUCCAUCAUC |
| | A-C | |
| | 23_6-6_internal_ | |
| | loop-symmetric_ | |
| | AUCCUA-GUCCUA | |
| 520 | -9_6-6_internal_ | GAUAUUGUGACAUAUCUCUGCACUCAU |
| | loop-symmetric_ | CACACAAAAGAUGCAAAUCUUCUAGAC |
| | UAGUUC-ACAGGU | AAACCACAGGGAGGGGGCAUCUUAAU |
| | 5_1-1_mismatch_ | AUAUCUCUACAGGUAUCAUC |
| | A-C | |
| | 23_6-6_internal_ | |
| | loop-symmetric_ | |
| | AUCCUA-GACAAA | |
| 521 | -9_6-6_internal_ | GAUAUUGUGACAUAUCUCUGCACUCAU |
| | loop-symmetric_ | CACACAAAAGAUGCAAAUCUUCUAGGC |
| | UAGUUC-ACCGUU | UGGCCACAGGGAGGGGGCAUUUUAAU |
| | 23_6-6_internal_ | AUAUCUCUACCGUUAUCAUC |
| | loop-symmetric_ | |
| | AUCCUA-GGCUGG | |
| 522 | -9_6-6_internal_ | GAUAUUGUGACAUAUCUCUGCACUCAU |
| | loop-symmetric_ | CACACAAAAGAUGCAAAUCUUCUUGAU |
| | UAGUUC-CACCGC | UCUCCACAGGGAGGGGGCAUUUUAACA |
| | 0_1-1_mismatch_ | UAUCUCUCACCGCAUCAUC |
| | A-C | |
| | 24_6-6_internal_ | |
| | loop-symmetric_ | |
| | UCCUAU-UGAUUC | |
| 523 | -9_6-6_internal_ | GAUAUUGUGACAUAUCUCUGCACUCAU |
| | loop-symmetric_ | CACACAAAAGAUGCAAAUCUUCUUUAC |
| | UAGUUC-UAGUAU | AUUCCACAGGGAGGGGGCAUUUCAAU |
| | 3_1-1_mismatch_ | AUAUCUCUUAGUAUAUCAUC |
| | A-C | |
| | 24_6-6_internal_ | |
| | loop-symmetric_ | |
| | UCCUAU-UUACAU | |
| 524 | -9_6-6_internal_ | GAUAUUGUGACAUAUCUCUGCACUCAU |
| | loop-symmetric_ | CACACAAAAGAUGCAAAUCUUCUUUCA |
| | UAGUUC-CCUCAU | UCUCCACAGGGAGGGGGCAUUCUAAUA |
| | 4_1-1_mismatch_ | UAUCUCUCCUCAUAUCAUC |
| | A-C | |
| | 24_6-6_internal_ | |
| | loop-symmetric_ | |
| | UCCUAU-UUCAUC | |
| 525 | -9_6-6_internal_ | GAUAUUGUGACAUAUCUCUGCACUCAU |
| | loop-symmetric_ | CACACAAAAGAUGCAAAUCUUCUCCAA |
| | UAGUUC-CCCGAU | UUUCCACAGGGAGGGGGCAUCUUAAU |
| | 5_1-1_mismatch_ | AUAUCUCUCCCGAUAUCAUC |
| | A-C | |
| | 24_6-6_internal_ | |
| | loop-symmetric_ | |
| | UCCUAU-CCAAUU | |
| 526 | -9_6-6_internal_ | GAUAUUGUGACAUAUCUCUGCACUCAU |
| | loop-symmetric_ | CACACAAAAGAUGCAAAUCUUCUUUAA |
| | UAGUUC-UUCCGC | UCUCCACAGGGAGGGGGCAUUUUAAU |
| | 24_6-6_internal_ | AUAUCUCUUUCCGCAUCAUC |
| | loop-symmetric_ | |
| | UCCUAU-UUAAUC | |
| 527 | -9_6-6_internal_ | GAUAUUGUGACAUAUCUCUGCACUCAU |
| | loop-symmetric_ | CACACAAAAGAUGCAAAUCUUCACUUU |
| | UAGUUC-CUCGUU | CAUCCACAGGGAGGGGGCAUUUUAACA |
| | 0_1-1_mismatch_ | UAUCUCUCUCGUUAUCAUC |
| | A-C | |
| | 25_6-6_internal_ | |
| | loop-symmetric_ | |
| | CCUAUA-ACUUUC | |
| 528 | -9_6-6_internal_ | GAUAUUGUGACAUAUCUCUGCACUCAU |
| | loop-symmetric_ | CACACAAAAGAUGCAAAUCUUCGUAUC |
| | UAGUUC-UUCCGC | CAUCCACAGGGAGGGGGCAUUCUAAUA |
| | 4_1-1_mismatch_ | UAUCUCUUUCCGCAUCAUC |
| | A-C | |
| | 25_6-6_internal_ | |
| | loop-symmetric_ | |
| | CCUAUA-GUAUCC | |
| 529 | -9_6-6_internal_ | GAUAUUGUGACAUAUCUCUGCACUCAU |
| | loop-symmetric_ | CACACAAAAGAUGCAAAUCUUCAUUCA |
| | UAGUUC-UGUUCC | UAUCCACAGGGAGGGGGCAUCUUAAU |
| | 5_1-1_mismatch_ | AUAUCUCUUGUUCCAUCAUC |
| | A-C | |
| | 25_6-6_internal_ | |
| | loop-symmetric_ | |
| | CCUAUA-AUUCAU | |
| 530 | -9_6-6_internal_ | GAUAUUGUGACAUAUCUCUGCACUCAU |
| | loop-symmetric_ | CACACAAAAGAUGCAAAUCUUCACUUC |
| | UAGUUC-UUAAAU | CAUCCACAGGGAGGGGGCAUUUUAAU |
| | 25_6-6_internal_ | AUAUCUCUUUAAAUAUCAUC |
| | loop-symmetric_ | |
| | CCUAUA-ACUUCC | |
| 531 | -9_6-6_internal_ | GAUAUUGUGACAUAUCUCUGCACUCAU |
| | loop-symmetric_ | CACACAAAAGAUGCAAAUCUUGACACC |
| | UAGUUC-ACCAAU | GAUCCACAGGGAGGGGGCAUUUCAAU |
| | 3_1-1_mismatch_ | AUAUCUCUACCAAUAUCAUC |
| | A-C | |
| | 26_6-6_internal_ | |
| | loop-symmetric_ | |
| | CUAUAG-GACACC | |
| 532 | -9_6-6_internal_ | GAUAUUGUGACAUAUCUCUGCACUCAU |
| | loop-symmetric_ | CACACAAAAGAUGCAAAUCUUGAGGUC |
| | UAGUUC-AGUUAC | GAUCCACAGGGAGGGGGCAUUCUAAU |
| | 4_1-1_mismatch_ | AUAUCUCUAGUUACAUCAUC |
| | A-C | |
| | 26_6-6_internal_ | |
| | loop-symmetric_ | |
| | CUAUAG-GAGGUC | |
| 533 | -9_6-6_internal_ | GAUAUUGUGACAUAUCUCUGCACUCAU |
| | loop-symmetric_ | CACACAAAAGAUGCAAAUCUUACUCUC |
| | UAGUUC-CGUUAC | GAUCCACAGGGAGGGGGCAUCUUAAU |
| | 5_1-1_mismatch_ | AUAUCUCUCGUUACAUCAUC |
| | A-C | |
| | 26_6-6_internal_ | |
| | loop-symmetric_ | |
| | CUAUAG-ACUCUC | |
| 534 | -9_6-6_internal_ | GAUAUUGUGACAUAUCUCUGCACUCAU |
| | loop-symmetric_ | CACACAAAAGAUGCAAAUCUUGCAAAU |
| | UAGUUC-CUUGGU | GAUCCACAGGGAGGGGGCAUUUUAAU |
| | 26_6-6_internal_ | AUAUCUCUCUUGGUAUCAUC |
| | loop-symmetric_ | |
| | CUAUAG-GCAAAU | |
| 535 | -9_6-6_internal_ | GAUAUUGUGACAUAUCUCUGCACUCAU |
| | loop-symmetric_ | CACACAAAAGAUGCAAAUCUCACUUCG |
| | UAGUUC-UUAGAU | GAUCCACAGGGAGGGGGCAUUUUAAC |
| | 0_1-1_mismatch_ | AUAUCUCUUUAGAUAUCAUC |
| | A-C | |
| | 27_6-6_internal_ | |
| | loop-symmetric_ | |
| | UAUAGA-CACUUC | |
| 536 | -9_6-6_internal_ | GAUAUUGUGACAUAUCUCUGCACUCAU |
| | loop-symmetric_ | CACACAAAAGAUGCAAAUCUGGCCUUG |
| | UAGUUC-UUUGCC | GAUCCACAGGGAGGGGGCAUUUCAAU |
| | 3_1-1_mismatch_ | AUAUCUCUUUUGCCAUCAUC |
| | A-C | |
| | 27_6-6_internal_ | |
| | loop-symmetric_ | |
| | UAUAGA-GGCCUU | |
| 537 | -9_6-6_internal_ | GAUAUUGUGACAUAUCUCUGCACUCAU |
| | loop-symmetric_ | CACACAAAAGAUGCAAAUCUCCCUCCG |
| | UAGUUC-AUCGCC | GAUCCACAGGGAGGGGGCAUUCUAAU |
| | 4_1-1_mismatch_ | AUAUCUCUAUCGCCAUCAUC |
| | A-C | |
| | 27_6-6_internal_ | |
| | loop-symmetric_ | |
| | UAUAGA-CCCUCC | |
| 538 | -9_6-6_internal_ | GAUAUUGUGACAUAUCUCUGCACUCAU |
| | loop-symmetric_ | CACACAAAAGAUGCAAAUCUAGACACG |
| | UAGUUC-AUUGUU | GAUCCACAGGGAGGGGGCAUCUUAAU |
| | 5_1-1_mismatch_ | AUAUCUCUAUUGUUAUCAUC |
| | A-C | |
| | 27_6-6_internal_ | |
| | loop-symmetric_ | |
| | UAUAGA-AGACAC | |
| 539 | -9_6-6_internal_ | GAUAUUGUGACAUAUCUCUGCACUCAU |
| | loop-symmetric_ | CACACAAAAGAUGCAAAUCUGUCCCUG |
| | UAGUUC-UGUAGU | GAUCCACAGGGAGGGGGCAUUUUAAU |
| | 27_6-6_internal_ | AUAUCUCUUGUAGUAUCAUC |
| | loop-symmetric_ | |
| | UAUAGA-GUCCCU | |
| 540 | -9_6-6_internal_ | GAUAUUGUGACAUAUCUCUGCACUCAU |
| | loop-symmetric_ | CACACAAAAGAUGCAAAUCGCGUUGAG |
| | UAGUUC-AUCCCU | GAUCCACAGGGAGGGGGCAUUUUAAC |
| | 0_1-1_mismatch_ | AUAUCUCUAUCCCUAUCAUC |
| | A-C | |
| | 28_6-6_internal_ | |
| | loop-symmetric_ | |
| | AUAGAA-GCGUUG | |
| 541 | -9_6-6_internal_ | GAUAUUGUGACAUAUCUCUGCACUCAU |
| | loop-symmetric_ | CACACAAAAGAUGCAAAUCCCGCACAG |
| | UAGUUC-UCCGAU | GAUCCACAGGGAGGGGGCAUUUCAAU |
| | 3_1-1_mismatch_ | AUAUCUCUUCCGAUAUCAUC |
| | A-C | |
| | 28_6-6_internal_ | |
| | loop-symmetric_ | |
| | AUAGAA-CCGCAC | |
| 542 | -9_6-6_internal_ | GAUAUUGUGACAUAUCUCUGCACUCAU |
| | loop-symmetric_ | CACACAAAAGAUGCAAAUCAGGAAGA |
| | UAGUUC-UUCAGU | GGAUCCACAGGGAGGGGGCAUUCUAA |
| | 4_1-1_mismatch_ | UAUAUCUCUUUCAGUAUCAUC |
| | A-C | |
| | 28_6-6_internal_ | |
| | loop-symmetric_ | |
| | AUAGAA-AGGAAG | |
| 543 | -9_6-6_internal_ | GAUAUUGUGACAUAUCUCUGCACUCAU |
| | loop-symmetric_ | CACACAAAAGAUGCAAAUCAAGCCCAG |
| | UAGUUC-AUCAAU | GAUCCACAGGGAGGGGGCAUCUUAAU |
| | 5_1-1_mismatch_ | AUAUCUCUAUCAAUAUCAUC |
| | A-C | |
| | 28_6-6_internal_ | |
| | loop-symmetric_ | |
| | AUAGAA-AAGCCC | |
| 544 | -9_6-6_internal_ | GAUAUUGUGACAUAUCUCUGCACUCAU |
| | loop-symmetric_ | CACACAAAAGAUGCAAAUCACAAGGAG |
| | UAGUUC-AUUUAU | GAUCCACAGGGAGGGGGCAUUUUAAU |
| | 28_6-6_internal_ | AUAUCUCUAUUUAUAUCAUC |
| | loop-symmetric_ | |
| | AUAGAA-ACAAGG | |
| 545 | -9_6-6_internal_ | GAUAUUGUGACAUAUCUCUGCACUCAU |
| | loop-symmetric_ | CACACAAAAGAUGCAAAUGAGAGCUA |
| | UAGUUC-UCAUAU | GGAUCCACAGGGAGGGGGCAUUUUAA |
| | 0_1-1_mismatch_ | CAUAUCUCUUCAUAUAUCAUC |
| | A-C | |
| | 29_6-6_internal_ | |
| | loop-symmetric_ | |
| | UAGAAG-GAGAGC | |
| 546 | -9_6-6_internal_ | GAUAUUGUGACAUAUCUCUGCACUCAU |
| | loop-symmetric_ | CACACAAAAGAUGCAAAUGGCGGUUA |
| | UAGUUC-UUUUCC | GGAUCCACAGGGAGGGGGCAUUUCAA |
| | 3_1-1_mismatch_ | UAUAUCUCUUUUUCCAUCAUC |
| | A-C | |
| | 29_6-6_internal_ | |
| | loop-symmetric_ | |
| | UAGAAG-GGCGGU | |
| 547 | -9_6-6_internal_ | GAUAUUGUGACAUAUCUCUGCACUCAU |
| | loop-symmetric_ | CACACAAAAGAUGCAAAUGGAGGUUA |
| | UAGUUC-CUAAAU | GGAUCCACAGGGAGGGGGCAUUCUAA |
| | 4_1-1_mismatch_ | UAUAUCUCUCUAAAUAUCAUC |
| | A-C | |
| | 29_6-6_internal_ | |
| | loop-symmetric_ | |
| | UAGAAG-GGAGGU | |
| 548 | -9_6-6_internal_ | GAUAUUGUGACAUAUCUCUGCACUCAU |
| | loop-symmetric_ | CACACAAAAGAUGCAAAUACCACUUAG |
| | UAGUUC-AUCCCC | GAUCCACAGGGAGGGGGCAUCUUAAU |
| | 5_1-1_mismatch_ | AUAUCUCUAUCCCCAUCAUC |
| | A-C | |
| | 29_6-6_internal_ | |
| | loop-symmetric_ | |
| | UAGAAG-ACCACU | |
| 549 | -9_6-6_internal_ | GAUAUUGUGACAUAUCUCUGCACUCAU |
| | loop-symmetric_ | CACACAAAAGAUGCAAAGCAGGCAUAG |
| | UAGUUC-CCAGUU | GAUCCACAGGGAGGGGGCAUUUCAAU |
| | 3_1-1_mismatch_ | AUAUCUCUCCAGUUAUCAUC |
| | A-C | |
| | 30_6-6_internal_ | |
| | loop-symmetric_ | |
| | AGAAGA-GCAGGC | |
| 550 | -9_6-6_internal_ | GAUAUUGUGACAUAUCUCUGCACUCAU |
| | loop-symmetric_ | CACACAAAAGAUGCAAAGAGAGCAUA |
| | UAGUUC-CCUGGU | GGAUCCACAGGGAGGGGGCAUUCUAA |
| | 4_1-1_mismatch_ | UAUAUCUCUCCUGGUAUCAUC |
| | A-C | |
| | 30_6-6_internal_ | |
| | loop-symmetric_ | |
| | AGAAGA-GAGAGC | |
| 551 | -9_6-6_internal_ | GAUAUUGUGACAUAUCUCUGCACUCAU |
| | loop-symmetric_ | CACACAAAAGAUGCAAACGAGGAAUA |
| | UAGUUC-CACCCC | GGAUCCACAGGGAGGGGGCAUCUUAA |
| | 5_1-1_mismatch_ | UAUAUCUCUCACCCCAUCAUC |
| | A-C | |
| | 30_6-6_internal_ | |
| | loop-symmetric_ | |
| | AGAAGA-CGAGGA | |
| 552 | -9_6-6_internal_ | GAUAUUGUGACAUAUCUCUGCACUCAU |
| | loop-symmetric_ | CACACAAAAGAUGCAAAAGCCGAAUAG |
| | UAGUUC-CAGUAU | GAUCCACAGGGAGGGGGCAUUUUAAU |
| | 30_6-6_internal_ | AUAUCUCUCAGUAUAUCAUC |
| | loop-symmetric_ | |
| | AGAAGA-AGCCGA | |
| 553 | -9_6-6_internal_ | GAUAUUGUGACAUAUCUCUGCACUCAU |
| | loop-symmetric_ | CACACAAAAGAUGCAACAACGAUAUAG |
| | UAGUUC-AUGUUC | GAUCCACAGGGAGGGGGCAUUUUAAC |
| | 0_1-1_mismatch_ | AUAUCUCUAUGUUCAUCAUC |
| | A-C | |
| | 31_6-6_internal_ | |
| | loop-symmetric_ | |
| | GAAGAU-CAACGA | |
| 554 | -9_6-6_internal_ | GAUAUUGUGACAUAUCUCUGCACUCAU |
| | loop-symmetric_ | CACACAAAAGAUGCAACCAACGUAUAG |
| | UAGUUC-CCCUUC | GAUCCACAGGGAGGGGGCAUUUCAAU |
| | 3_1-1_mismatch_ | AUAUCUCUCCCUUCAUCAUC |
| | A-C | |
| | 31_6-6_internal_ | |
| | loop-symmetric_ | |
| | GAAGAU-CCAACG | |
| 555 | -9_6-6_internal_ | GAUAUUGUGACAUAUCUCUGCACUCAU |
| | loop-symmetric_ | CACACAAAAGAUGCAAUGACAAUAUA |
| | UAGUUC-CCAAAU | GGAUCCACAGGGAGGGGGCAUUCUAA |
| | 4_1-1_mismatch_ | UAUAUCUCUCCAAAUAUCAUC |
| | A-C | |
| | 31_6-6_internal_ | |
| | loop-symmetric_ | |
| | GAAGAU-UGACAA | |
| 556 | -9_6-6_internal_ | GAUAUUGUGACAUAUCUCUGCACUCAU |
| | loop-symmetric_ | CACACAAAAGAUGCAACUGGUAUAUA |
| | UAGUUC-CGCCGC | GGAUCCACAGGGAGGGGGCAUCUUAA |
| | 5_1-1_mismatch_ | UAUAUCUCUCGCCGCAUCAUC |
| | A-C | |
| | 31_6-6_internal_ | |
| | loop-symmetric_ | |
| | GAAGAU-CUGGUA | |
| 557 | -9_6-6_internal_ | GAUAUUGUGACAUAUCUCUGCACUCAU |
| | loop-symmetric_ | CACACAAAAGAUGCAAUGACGAUAUA |
| | UAGUUC-UAUAAU | GGAUCCACAGGGAGGGGGCAUUUUAA |
| | 31_6-6_internal_ | UAUAUCUCUUAUAAUAUCAUC |
| | loop-symmetric_ | |
| | GAAGAU-UGACGA | |
| 558 | -9_6-6_internal_ | GAUAUUGUGACAUAUCUCUGCACUCAU |
| | loop-symmetric_ | CACACAAAAGAUGCAUAAGGGCUAUA |
| | UAGUUC-UUUAAU | GGAUCCACAGGGAGGGGGCAUUUUAA |
| | 0_1-1_mismatch_ | CAUAUCUCUUUUAAUAUCAUC |
| | A-C | |
| | 32_6-6_internal_ | |
| | loop-symmetric_ | |
| | AAGAUU-UAAGGG | |
| 559 | -9_6-6_internal_ | GAUAUUGUGACAUAUCUCUGCACUCAU |
| | loop-symmetric_ | CACACAAAAGAUGCAUUCAUCCUAUAG |
| | UAGUUC-CCAAGU | GAUCCACAGGGAGGGGGCAUUCUAAU |
| | 4_1-1_mismatch_ | AUAUCUCUCCAAGUAUCAUC |
| | A-C | |
| | 32_6-6_internal_ | |
| | loop-symmetric_ | |
| | AAGAUU-UUCAUC | |
| 560 | -9_6-6_internal_ | GAUAUUGUGACAUAUCUCUGCACUCAU |
| | loop-symmetric_ | CACACAAAAGAUGCACCGAAACUAUAG |
| | UAGUUC-AGUGAU | GAUCCACAGGGAGGGGGCAUCUUAAU |
| | 5_1-1_mismatch_ | AUAUCUCUAGUGAUAUCAUC |
| | A-C | |
| | 32_6-6_internal_ | |
| | loop-symmetric_ | |
| | AAGAUU-CCGAAA | |
| 561 | -9_6-6_internal_ | GAUAUUGUGACAUAUCUCUGCACUCAU |
| | loop-symmetric_ | CACACAAAAGAUGCAUGGAGACUAUA |
| | UAGUUC-UAUUUU | GGAUCCACAGGGAGGGGGCAUUUUAA |
| | 32_6-6_internal_ | UAUAUCUCUUAUUUUAUCAUC |
| | loop-symmetric_ | |
| | AAGAUU-UGGAGA | |
| 562 | -9_6-6_internal_ | GAUAUUGUGACAUAUCUCUGCACUCAU |
| | loop-symmetric_ | CACACAAAAGAUGCCCCAUGUCUAUAG |
| | UAGUUC-UUCCCU | GAUCCACAGGGAGGGGGCAUUUUAAC |
| | 0_1-1_mismatch_ | AUAUCUCUUUCCCUAUCAUC |
| | A-C | |
| | 33_6-6_internal_ | |
| | loop-symmetric_ | |
| | AGAUUU-CCCAUG | |
| 563 | -9_6-6_internal_ | GAUAUUGUGACAUAUCUCUGCACUCAU |
| | loop-symmetric_ | CACACAAAAGAUGCCAUAACUCUAUAG |
| | UAGUUC-ACCUUU | GAUCCACAGGGAGGGGGCAUUCUAAU |
| | 4_1-1_mismatch_ | AUAUCUCUACCUUUAUCAUC |
| | A-C | |
| | 33_6-6_internal_ | |
| | loop-symmetric_ | |
| | AGAUUU-CAUAAC | |
| 564 | -9_6-6_internal_ | GAUAUUGUGACAUAUCUCUGCACUCAU |
| | loop-symmetric_ | CACACAAAAGAUGCCUACAGUCUAUAG |
| | UAGUUC-AUUUAC | GAUCCACAGGGAGGGGGCAUCUUAAU |
| | 5_1-1_mismatch_ | AUAUCUCUAUUUACAUCAUC |
| | A-C | |
| | 33_6-6_internal_ | |
| | loop-symmetric_ | |
| | AGAUUU-CUACAG | |
| 565 | -9_6-6_internal_ | GAUAUUGUGACAUAUCUCUGCACUCAU |
| | loop-symmetric_ | CACACAAAAGAUGCUUGCCGUCUAUAG |
| | UAGUUC-AUUAAU | GAUCCACAGGGAGGGGGCAUUUUAAU |
| | 33_6-6_internal_ | AUAUCUCUAUUAAUAUCAUC |
| | loop-symmetric_ | |
| | AGAUUU-UUGCCG | |
| 566 | -9_6-6_internal_ | GAUAUUGUGACAUAUCUCUGCACUCAU |
| | loop-symmetric_ | CACACAAAAGAUGAUUGGGUUCUAUA |
| | UAGUUC-UCCACC | GGAUCCACAGGGAGGGGGCAUUUCAA |
| | 3_1-1_mismatch_ | UAUAUCUCUUCCACCAUCAUC |
| | A-C | |
| | 34_6-6_internal_ | |
| | loop-symmetric_ | |
| | GAUUUG-AUUGGG | |
| 567 | -9_6-6_internal_ | GAUAUUGUGACAUAUCUCUGCACUCAU |
| | loop-symmetric_ | CACACAAAAGAUGGUAUGGUUCUAUA |
| | UAGUUC-UCUUAC | GGAUCCACAGGGAGGGGGCAUUCUAA |
| | 4_1-1_mismatch_ | UAUAUCUCUUCUUACAUCAUC |
| | A-C | |
| | 34_6-6_internal_ | |
| | loop-symmetric_ | |
| | GAUUUG-GUAUGG | |
| 568 | -9_6-6_internal_ | GAUAUUGUGACAUAUCUCUGCACUCAU |
| | loop-symmetric_ | CACACAAAAGAUGACCAGGUUCUAUAG |
| | UAGUUC-UUUGGU | GAUCCACAGGGAGGGGGCAUCUUAAU |
| | 5_1-1_mismatch_ | AUAUCUCUUUUGGUAUCAUC |
| | A-C | |
| | 34_6-6_internal_ | |
| | loop-symmetric_ | |
| | GAUUUG-ACCAGG | |
| 569 | -9_6-6_internal_ | GAUAUUGUGACAUAUCUCUGCACUCAU |
| | loop-symmetric_ | CACACAAAAGAUGAGCGAAUUCUAUA |
| | UAGUUC-UUUUAC | GGAUCCACAGGGAGGGGGCAUUUUAA |
| | 34_6-6_internal_ | UAUAUCUCUUUUUACAUCAUC |
| | loop-symmetric_ | |
| | GAUUUG-AGCGAA | |
| 570 | -9_6-6_internal_ | GAUAUUGUGACAUAUCUCUGCACUCAU |
| | loop-symmetric_ | CACACAAAAGAUAAUGGACUUCUAUA |
| | UAGUUC-UAUAAU | GGAUCCACAGGGAGGGGGCAUUUUAA |
| | 0_1-1_mismatch_ | CAUAUCUCUUAUAAUAUCAUC |
| | A-C | |
| | 35_6-6_internal_ | |
| | loop-symmetric_ | |
| | AUUUGC-AAUGGA | |
| 571 | -9_6-6_internal_ | GAUAUUGUGACAUAUCUCUGCACUCAU |
| | loop-symmetric_ | CACACAAAAGAUAUCGUGCUUCUAUAG |
| | UAGUUC-AUUCAU | GAUCCACAGGGAGGGGGCAUUUCAAU |
| | 3_1-1_mismatch_ | AUAUCUCUAUUCAUAUCAUC |
| | A-C | |
| | 35_6-6_internal_ | |
| | loop-symmetric_ | |
| | AUUUGC-AUCGUG | |
| 572 | -9_6-6_internal_ | GAUAUUGUGACAUAUCUCUGCACUCAU |
| | loop-symmetric_ | CACACAAAAGAUUUCGUGCUUCUAUAG |
| | UAGUUC-ACCCGC | GAUCCACAGGGAGGGGGCAUUCUAAU |
| | 4_1-1_mismatch_ | AUAUCUCUACCCGCAUCAUC |
| | A-C | |
| | 35_6-6_internal_ | |
| | loop-symmetric_ | |
| | AUUUGC-UUCGUG | |
| 573 | -9_6-6_internal_ | GAUAUUGUGACAUAUCUCUGCACUCAU |
| | loop-symmetric_ | CACACAAAAGAUAUCCGACUUCUAUAG |
| | UAGUUC-CACCAC | GAUCCACAGGGAGGGGGCAUCUUAAU |
| | 5_1-1_mismatch_ | AUAUCUCUCACCACAUCAUC |
| | A-C | |
| | 35_6-6_internal_ | |
| | loop-symmetric_ | |
| | AUUUGC-AUCCGA | |
| 574 | -9_6-6_internal_ | GAUAUUGUGACAUAUCUCUGCACUCAU |
| | loop-symmetric_ | CACACAAAAGAUUCCUACCUUCUAUAG |
| | UAGUUC-CUCGGU | GAUCCACAGGGAGGGGGCAUUUUAAU |
| | 35_6-6_internal_ | AUAUCUCUCUCGGUAUCAUC |
| | loop-symmetric_ | |
| | AUUUGC-UCCUAC | |
| 575 | -9_6-6_internal_ | GAUAUUGUGACAUAUCUCUGCACUCAU |
| | loop-symmetric_ | CACACAAAAGACCAUUCUCUUCUAUAG |
| | UAGUUC-UCCCCU | GAUCCACAGGGAGGGGGCAUUUUAAC |
| | 0_1-1_mismatch_ | AUAUCUCUUCCCCUAUCAUC |
| | A-C | |
| | 36_6-6_internal_ | |
| | loop-symmetric_ | |
| | UUUGCA-CCAUUC | |
| 576 | -9_6-6_internal_ | GAUAUUGUGACAUAUCUCUGCACUCAU |
| | loop-symmetric_ | CACACAAAAGAAAAACCUCUUCUAUAG |
| | UAGUUC-UCAAAU | GAUCCACAGGGAGGGGGCAUUUCAAU |
| | 3_1-1_mismatch_ | AUAUCUCUUCAAAUAUCAUC |
| | A-C | |
| | 36_6-6_internal_ | |
| | loop-symmetric_ | |
| | UUUGCA-AAAACC | |
| 577 | -9_6-6_internal_ | GAUAUUGUGACAUAUCUCUGCACUCAU |
| | loop-symmetric_ | CACACAAAAGACCGUUUUCUUCUAUAG |
| | UAGUUC-CCUUAU | GAUCCACAGGGAGGGGGCAUUCUAAU |
| | 4_1-1_mismatch_ | AUAUCUCUCCUUAUAUCAUC |
| | A-C | |
| | 36_6-6_internal_ | |
| | loop-symmetric_ | |
| | UUUGCA-CCGUUU | |
| 578 | -9_6-6_internal_ | GAUAUUGUGACAUAUCUCUGCACUCAU |
| | loop-symmetric_ | CACACAAAAGAGAGUCCUCUUCUAUAG |
| | UAGUUC-AUCUCC | GAUCCACAGGGAGGGGGCAUCUUAAU |
| | 5_1-1_mismatch_ | AUAUCUCUAUCUCCAUCAUC |
| | A-C | |
| | 36_6-6_internal_ | |
| | loop-symmetric_ | |
| | UUUGCA-GAGUCC | |
| 579 | -9_6-6_internal_ | GAUAUUGUGACAUAUCUCUGCACUCAU |
| | loop-symmetric_ | CACACAAAAGCUUGAUAUCUUCUAUAG |
| | UAGUUC-CCUUGC | GAUCCACAGGGAGGGGGCAUUUUAAC |
| | 0_1-1_mismatch_ | AUAUCUCUCCUUGCAUCAUC |
| | A-C | |
| | 37_6-6_internal_ | |
| | loop-symmetric_ | |
| | UUGCAU-CUUGAU | |
| 580 | -9_6-6_internal_ | GAUAUUGUGACAUAUCUCUGCACUCAU |
| | loop-symmetric_ | CACACAAAAGCGAACUAUCUUCUAUAG |
| | UAGUUC-CCCUCC | GAUCCACAGGGAGGGGGCAUUUCAAU |
| | 3_1-1_mismatch_ | AUAUCUCUCCCUCCAUCAUC |
| | A-C | |
| | 37_6-6_internal_ | |
| | loop-symmetric_ | |
| | UUGCAU-CGAACU | |
| 581 | -9_6-6_internal_ | GAUAUUGUGACAUAUCUCUGCACUCAU |
| | loop-symmetric_ | CACACAAAAGCUAUCCAUCUUCUAUAG |
| | UAGUUC-UCUUGC | GAUCCACAGGGAGGGGGCAUUCUAAU |
| | 4_1-1_mismatch_ | AUAUCUCUUCUUGCAUCAUC |
| | A-C | |
| | 37_6-6_internal_ | |
| | loop-symmetric_ | |
| | UUGCAU-CUAUCC | |
| 582 | -9_6-6_internal_ | GAUAUUGUGACAUAUCUCUGCACUCAU |
| | loop-symmetric_ | CACACAAAAGUCAGUUAUCUUCUAUAG |
| | UAGUUC-CGCAGU | GAUCCACAGGGAGGGGGCAUCUUAAU |
| | 5_1-1_mismatch_ | AUAUCUCUCGCAGUAUCAUC |
| | A-C | |
| | 37_6-6_internal_ | |
| | loop-symmetric_ | |
| | UUGCAU-UCAGUU | |
| 583 | -9_6-6_internal_ | GAUAUUGUGACAUAUCUCUGCACUCAU |
| | loop-symmetric_ | CACACAAAAGUUUGACAUCUUCUAUAG |
| | UAGUUC-AUUAUU | GAUCCACAGGGAGGGGGCAUUUUAAU |
| | 37_6-6_internal_ | AUAUCUCUAUUAUUAUCAUC |
| | loop-symmetric_ | |
| | UUGCAU-UUUGAC | |
| 584 | -9_6-6_internal_ | GAUAUUGUGACAUAUCUCUGCACUCAU |
| | loop-symmetric_ | CACACAAAACUAUCUAAUCUUCUAUAG |
| | UAGUUC-CCUUCC | GAUCCACAGGGAGGGGGCAUUUUAAC |
| | 0_1-1_mismatch_ | AUAUCUCUCCUUCCAUCAUC |
| | A-C | |
| | 38_6-6_internal_ | |
| | loop-symmetric_ | |
| | UGCAUC-CUAUCU | |
| 585 | -9_6-6_internal_ | GAUAUUGUGACAUAUCUCUGCACUCAU |
| | loop-symmetric_ | CACACAAAACAGAAUAAUCUUCUAUAG |
| | UAGUUC-UUCGCC | GAUCCACAGGGAGGGGGCAUUUCAAU |
| | 3_1-1_mismatch_ | AUAUCUCUUUCGCCAUCAUC |
| | A-C | |
| | 38_6-6_internal_ | |
| | loop-symmetric_ | |
| | UGCAUC-CAGAAU | |
| 586 | -9_6-6_internal_ | GAUAUUGUGACAUAUCUCUGCACUCAU |
| | loop-symmetric_ | CACACAAAAUCCAAUAAUCUUCUAUAG |
| | UAGUUC-UCCUCC | GAUCCACAGGGAGGGGGCAUUCUAAU |
| | 4_1-1_mismatch_ | AUAUCUCUUCCUCCAUCAUC |
| | A-C | |
| | 38_6-6_internal_ | |
| | loop-symmetric_ | |
| | UGCAUC-UCCAAU | |
| 587 | -9_6-6_internal_ | GAUAUUGUGACAUAUCUCUGCACUCAU |
| | loop-symmetric_ | CACACAAAACCCAAUAAUCUUCUAUAG |
| | UAGUUC-ACAGGU | GAUCCACAGGGAGGGGGCAUCUUAAU |
| | 5_1-1_mismatch_ | AUAUCUCUACAGGUAUCAUC |
| | A-C | |
| | 38_6-6_internal_ | |
| | loop-symmetric_ | |
| | UGCAUC-CCCAAU | |
| 588 | -9_6-6_internal_ | GAUAUUGUGACAUAUCUCUGCACUCAU |
| | loop-symmetric_ | CACACAAAACUUAUCAAUCUUCUAUAG |
| | UAGUUC-UUCGGU | GAUCCACAGGGAGGGGGCAUUUUAAU |
| | 38_6-6_internal_ | AUAUCUCUUUCGGUAUCAUC |
| | loop-symmetric_ | |
| | UGCAUC-CUUAUC | |
| 589 | -9_6-6_internal_ | GAUAUUGUGACAUAUCUCUGCACUCAU |
| | loop-symmetric_ | CACACAAAUUUAAAAAAUCUUCUAUA |
| | UAGUUC-ACCCAC | GGAUCCACAGGGAGGGGGCAUUUUAA |
| | 0_1-1_mismatch_ | CAUAUCUCUACCCACAUCAUC |
| | A-C | |
| | 39_6-6_internal_ | |
| | loop-symmetric_ | |
| | GCAUCU-UUUAAA | |
| 590 | -9_6-6_internal_ | GAUAUUGUGACAUAUCUCUGCACUCAU |
| | loop-symmetric_ | CACACAAACACCGAAAAUCUUCUAUAG |
| | UAGUUC-UAGUCC | GAUCCACAGGGAGGGGGCAUUUCAAU |
| | 3_1-1_mismatch_ | AUAUCUCUUAGUCCAUCAUC |
| | A-C | |
| | 39_6-6_internal_ | |
| | loop-symmetric_ | |
| | GCAUCU-CACCGA | |
| 591 | -9_6-6_internal_ | GAUAUUGUGACAUAUCUCUGCACUCAU |
| | loop-symmetric_ | CACACAAAUAAAUAAAAUCUUCUAUA |
| | UAGUUC-CAUGCC | GGAUCCACAGGGAGGGGGCAUCUUAA |
| | 5_1-1_mismatch_ | UAUAUCUCUCAUGCCAUCAUC |
| | A-C | |
| | 39_6-6_internal_ | |
| | loop-symmetric_ | |
| | GCAUCU-UAAAUA | |
| 592 | -9_6-6_internal_ | GAUAUUGUGACAUAUCUCUGCACUCAU |
| | loop-symmetric_ | CACACAAACUUACGAAAUCUUCUAUAG |
| | UAGUUC-CCGUUU | GAUCCACAGGGAGGGGGCAUUUUAAU |
| | 39_6-6_internal_ | AUAUCUCUCCGUUUAUCAUC |
| | loop-symmetric_ | |
| | GCAUCU-CUUACG | |
| 593 | -9_6-6_internal_ | GAUAUUGUGACAUAUCUCUGCACUCAU |
| | loop-symmetric_ | CACACAACCCUCCCAAAUCUUCUAUAG |
| | UAGUUC-CGUGAU | GAUCCACAGGGAGGGGGCAUUUUAAC |
| | 0_1-1_mismatch_ | AUAUCUCUCGUGAUAUCAUC |
| | A-C | |
| | 40_6-6_internal_ | |
| | loop-symmetric_ | |
| | CAUCUU-CCCUCC | |
| 594 | -9_6-6_internal_ | GAUAUUGUGACAUAUCUCUGCACUCAU |
| | loop-symmetric_ | CACACAACCUUCCCAAAUCUUCUAUAG |
| | UAGUUC-UCUUGC | GAUCCACAGGGAGGGGGCAUUUCAAU |
| | 3_1-1_mismatch_ | AUAUCUCUUCUUGCAUCAUC |
| | A-C | |
| | 40_6-6_internal_ | |
| | loop-symmetric_ | |
| | CAUCUU-CCUUCC | |
| 595 | -9_6-6_internal_ | GAUAUUGUGACAUAUCUCUGCACUCAU |
| | loop-symmetric_ | CACACAAUUCCAACAAAUCUUCUAUAG |
| | UAGUUC-AUCCAU | GAUCCACAGGGAGGGGGCAUUCUAAU |
| | 4_1-1_mismatch_ | AUAUCUCUAUCCAUAUCAUC |
| | A-C | |
| | 40_6-6_internal_ | |
| | loop-symmetric_ | |
| | CAUCUU-UUCCAA | |
| 596 | -9_6-6_internal_ | GAUAUUGUGACAUAUCUCUGCACUCAU |
| | loop-symmetric_ | CACACAACGCGACCAAAUCUUCUAUAG |
| | UAGUUC-UAUGAU | GAUCCACAGGGAGGGGGCAUCUUAAU |
| | 5_1-1_mismatch_ | AUAUCUCUUAUGAUAUCAUC |
| | A-C | |
| | 40_6-6_internal_ | |
| | loop-symmetric_ | |
| | CAUCUU-CGCGAC | |
| 597 | -9_6-6_internal_ | GAUAUUGUGACAUAUCUCUGCACUCAU |
| | loop-symmetric_ | CACACAACCACAUCAAAUCUUCUAUAG |
| | UAGUUC-CCUGUU | GAUCCACAGGGAGGGGGCAUUUUAAU |
| | 40_6-6_internal_ | AUAUCUCUCCUGUUAUCAUC |
| | loop-symmetric_ | |
| | CAUCUU-CCACAU | |
| 598 | -9_6-6_internal_ | GAUAUUGUGACAUAUCUCUGCACUCAU |
| | loop-symmetric_ | CACACACACAUGGCAAAUCUUCUAUAG |
| | UAGUUC-CCGGGU | GAUCCACAGGGAGGGGGCAUUUUAAC |
| | 0_1-1_mismatch_ | AUAUCUCUCCGGGUAUCAUC |
| | A-C | |
| | 41_6-6_internal_ | |
| | loop-symmetric_ | |
| | AUCUUU-CACAUG | |
| 599 | -9_6-6_internal_ | GAUAUUGUGACAUAUCUCUGCACUCAU |
| | loop-symmetric_ | CACACACUGUUAGCAAAUCUUCUAUAG |
| | UAGUUC-AUCCGC | GAUCCACAGGGAGGGGGCAUUUCAAU |
| | 3_1-1_mismatch_ | AUAUCUCUAUCCGCAUCAUC |
| | A-C | |
| | 41_6-6_internal_ | |
| | loop-symmetric_ | |
| | AUCUUU-CUGUUA | |
| 600 | -9_6-6_internal_ | GAUAUUGUGACAUAUCUCUGCACUCAU |
| | loop-symmetric_ | CACACACUAUCAGCAAAUCUUCUAUAG |
| | UAGUUC-CACCGC | GAUCCACAGGGAGGGGGCAUUCUAAU |
| | 4_1-1_mismatch_ | AUAUCUCUCACCGCAUCAUC |
| | A-C | |
| | 41_6-6_internal_ | |
| | loop-symmetric_ | |
| | AUCUUU-CUAUCA | |
| 601 | -9_6-6_internal_ | GAUAUUGUGACAUAUCUCUGCACUCAU |
| | loop-symmetric_ | CACACACUUCAAGCAAAUCUUCUAUAG |
| | UAGUUC-CCCCGC | GAUCCACAGGGAGGGGGCAUCUUAAU |
| | 5_1-1_mismatch_ | AUAUCUCUCCCCGCAUCAUC |
| | A-C | |
| | 41_6-6_internal_ | |
| | loop-symmetric_ | |
| | AUCUUU-CUUCAA | |
| 602 | -9_6-6_internal_ | GAUAUUGUGACAUAUCUCUGCACUCAU |
| | loop-symmetric_ | CACACACAUCGCGCAAAUCUUCUAUAG |
| | UAGUUC-UUCGGU | GAUCCACAGGGAGGGGGCAUUUUAAU |
| | 41_6-6_internal_ | AUAUCUCUUUCGGUAUCAUC |
| | loop-symmetric_ | |
| | AUCUUU-CAUCGC | |
| 603 | -9_6-6_internal_ | GAUAUUGUGACAUAUCUCUGCACUCAU |
| | loop-symmetric_ | CACACUGCGAUUGCAAAUCUUCUAUAG |
| | UAGUUC-UUCCGC | GAUCCACAGGGAGGGGGCAUUUUAAC |
| | 0_1-1_mismatch_ | AUAUCUCUUUCCGCAUCAUC |
| | A-C | |
| | 42_6-6_internal_ | |
| | loop-symmetric_ | |
| | UCUUUU-UGCGAU | |
| 604 | -9_6-6_internal_ | GAUAUUGUGACAUAUCUCUGCACUCAU |
| | loop-symmetric_ | CACACUGUCUCUGCAAAUCUUCUAUAG |
| | UAGUUC-CCUUGC | GAUCCACAGGGAGGGGGCAUUUCAAU |
| | 3_1-1_mismatch_ | AUAUCUCUCCUUGCAUCAUC |
| | A-C | |
| | 42_6-6_internal_ | |
| | loop-symmetric_ | |
| | UCUUUU-UGUCUC | |
| 605 | -9_6-6_internal_ | GAUAUUGUGACAUAUCUCUGCACUCAU |
| | loop-symmetric_ | CACACCCAGUUUGCAAAUCUUCUAUAG |
| | UAGUUC-UCGGUU | GAUCCACAGGGAGGGGGCAUCUUAAU |
| | 5_1-1_mismatch_ | AUAUCUCUUCGGUUAUCAUC |
| | A-C | |
| | 42_6-6_internal_ | |
| | loop-symmetric_ | |
| | UCUUUU-CCAGUU | |
| 606 | -9_6-6_internal_ | GAUAUUGUGACAUAUCUCUGCACUCAU |
| | loop-symmetric_ | CACACCCUCACUGCAAAUCUUCUAUAG |
| | UAGUUC-AUUUUU | GAUCCACAGGGAGGGGGCAUUUUAAU |
| | 42_6-6_internal_ | AUAUCUCUAUUUUUAUCAUC |
| | loop-symmetric_ | |
| | UCUUUU-CCUCAC | |
| 607 | -9_6-6_internal_ | GAUAUUGUGACAUAUCUCUGCACUCAU |
| | loop-symmetric_ | CACAGCCAUCAUGCAAAUCUUCUAUAG |
| | UAGUUC-CUGUCC | GAUCCACAGGGAGGGGGCAUUUUAAC |
| | 0_1-1_mismatch_ | AUAUCUCUCUGUCCAUCAUC |
| | A-C | |
| | 43_6-6_internal_ | |
| | loop-symmetric_ | |
| | CUUUUG-GCCAUC | |
| 608 | -9_6-6_internal_ | GAUAUUGUGACAUAUCUCUGCACUCAU |
| | loop-symmetric_ | CACAAAUACUAUGCAAAUCUUCUAUAG |
| | UAGUUC-UUCAGU | GAUCCACAGGGAGGGGGCAUUUCAAU |
| | 3_1-1_mismatch_ | AUAUCUCUUUCAGUAUCAUC |
| | A-C | |
| | 43_6-6_internal_ | |
| | loop-symmetric_ | |
| | CUUUUG-AAUACU | |
| 609 | -9_6-6_internal_ | GAUAUUGUGACAUAUCUCUGCACUCAU |
| | loop-symmetric_ | CACAACUCAUAUGCAAAUCUUCUAUAG |
| | UAGUUC-AUUUUU | GAUCCACAGGGAGGGGGCAUUCUAAU |
| | 4_1-1_mismatch_ | AUAUCUCUAUUUUUAUCAUC |
| | A-C | |
| | 43_6-6_internal_ | |
| | loop-symmetric_ | |
| | CUUUUG-ACUCAU | |
| 610 | -9_6-6_internal_ | GAUAUUGUGACAUAUCUCUGCACUCAU |
| | loop-symmetric_ | CACAAUGGCAAUGCAAAUCUUCUAUAG |
| | UAGUUC-CGCCCC | GAUCCACAGGGAGGGGGCAUCUUAAU |
| | 5_1-1_mismatch_ | AUAUCUCUCGCCCCAUCAUC |
| | A-C | |
| | 43_6-6_internal_ | |
| | loop-symmetric_ | |
| | CUUUUG-AUGGCA | |
| 611 | -9_6-6_internal_ | GAUAUUGUGACAUAUCUCUGCACUCAU |
| | loop-symmetric_ | CACAAAAUCAAUGCAAAUCUUCUAUAG |
| | UAGUUC-CUUAGU | GAUCCACAGGGAGGGGGCAUUUUAAU |
| | 43_6-6_internal_ | AUAUCUCUCUUAGUAUCAUC |
| | loop-symmetric_ | |
| | CUUUUG-AAAUCA | |
| 612 | -9_6-6_internal_ | GAUAUUGUGACAUAUCUCUGCACUCAU |
| | loop-symmetric_ | CACUUGUCCGAUGCAAAUCUUCUAUAG |
| | UAGUUC-AGUAGU | GAUCCACAGGGAGGGGGCAUUUUAAC |
| | 0_1-1_mismatch_ | AUAUCUCUAGUAGUAUCAUC |
| | A-C | |
| | 44_6-6_internal_ | |
| | loop-symmetric_ | |
| | UUUUGU-UUGUCC | |
| 613 | -9_6-6_internal_ | GAUAUUGUGACAUAUCUCUGCACUCAU |
| | loop-symmetric_ | CACUAUACCGAUGCAAAUCUUCUAUAG |
| | UAGUUC-CUCCGC | GAUCCACAGGGAGGGGGCAUUUCAAU |
| | 3_1-1_mismatch_ | AUAUCUCUCUCCGCAUCAUC |
| | A-C | |
| | 44_6-6_internal_ | |
| | loop-symmetric_ | |
| | UUUUGU-UAUACC | |
| 614 | -9_6-6_internal_ | GAUAUUGUGACAUAUCUCUGCACUCAU |
| | loop-symmetric_ | CACUAUUUCGAUGCAAAUCUUCUAUAG |
| | UAGUUC-UCCCAC | GAUCCACAGGGAGGGGGCAUUCUAAU |
| | 4_1-1_mismatch_ | AUAUCUCUUCCCACAUCAUC |
| | A-C | |
| | 44_6-6_internal_ | |
| | loop-symmetric_ | |
| | UUUUGU-UAUUUC | |
| 615 | -9_6-6_internal_ | GAUAUUGUGACAUAUCUCUGCACUCAU |
| | loop-symmetric_ | CACUGAUAUGAUGCAAAUCUUCUAUA |
| | UAGUUC-AUUGCC | GGAUCCACAGGGAGGGGGCAUCUUAA |
| | 5_1-1_mismatch_ | UAUAUCUCUAUUGCCAUCAUC |
| | A-C | |
| | 44_6-6_internal_ | |
| | loop-symmetric_ | |
| | UUUUGU-UGAUAU | |
| 616 | -9_6-6_internal_ | GAUAUUGUGACAUAUCUCUGCACUCAU |
| | loop-symmetric_ | CACUAUUACGAUGCAAAUCUUCUAUAG |
| | UAGUUC-AUCACC | GAUCCACAGGGAGGGGGCAUUUUAAU |
| | 44_6-6_internal_ | AUAUCUCUAUCACCAUCAUC |
| | loop-symmetric_ | |
| | UUUUGU-UAUUAC | |
| 617 | -8_6-6_internal_ | GAUAUUGUGACAUAUCUCUGCACUCAU |
| | loop-symmetric_ | CACACAAAAGAUGCAAAUCUUCUAUAG |
| | AGUUCA-CUUCGG | GAUCCACAGGGAGGGGGCAUUUUAAC |
| | 0_1-1_mismatch_ | AUAUCUCCUUCGGAAUCAUC |
| | A-C | |
| 618 | -8_6-6_internal_ | GAUAUUGUGACAUAUCUCUGCACUCAU |
| | loop-symmetric_ | CACACAAAAGAUGCAAAUCUUCUAUAG |
| | AGUUCA-AUCUAA | GAUCCACAGGGAGGGGGCAUUUCAAU |
| | 3_1-1_mismatch_ | AUAUCUCAUCUAAAAUCAUC |
| | A-C | |
| 619 | -8_6-6_internal_ | GAUAUUGUGACAUAUCUCUGCACUCAU |
| | loop-symmetric_ | CACACAAAAGAUGCAAAUCUUCUAUAG |
| | AGUUCA-CCACAA | GAUCCACAGGGAGGGGGCAUUCUAAU |
| | 4_1-1_mismatch_ | AUAUCUCCCACAAAAUCAUC |
| | A-C | |
| 620 | -8_6-6_internal_ | GAUAUUGUGACAUAUCUCUGCACUCAU |
| | loop-symmetric_ | CACACAAAAGAUGCAAAUCUUCUAUAG |
| | AGUUCA-GGUGAG | GAUCCACAGGGAGGGGGCAUCUUAAU |
| | 5_1-1_mismatch_ | AUAUCUCGGUGAGAAUCAUC |
| | A-C | |
| 621 | -8_6-6_internal_ | GAUAUUGUGACAUAUCUCUGCACUCAU |
| | loop-symmetric_ | CACACAAAAGAUGCAAAUCUUCUAUAG |
| | AGUUCA-CCCUGG | GAUCCACAGGGAGGGGGCAUUUUAAU |
| | | AUAUCUCCCCUGGAAUCAUC |
| 622 | -8_6-6_internal_ | GAUAUUGUGACAUAUCUCUGCACUCAU |
| | loop-symmetric_ | CACACAAAAGAUGCAAAUCUUCUAUAG |
| | AGUUCA-AUUUCG | CAAAUUCAGGGAGGGGGCAUUUUAAC |
| | 0_1-1_mismatch_ | AUAUCUCAUUUCGAAUCAUC |
| | A-C | |
| | 20_6-6_internal_ | |
| | loop-symmetric_ | |
| | UGGAUC-CAAAUU | |
| 623 | -8_6-6_internal_ | GAUAUUGUGACAUAUCUCUGCACUCAU |
| | loop-symmetric_ | CACACAAAAGAUGCAAAUCUUCUAUAG |
| | AGUUCA-AACAAG | UGCAGUCAGGGAGGGGGCAUUUCAAU |
| | 3_1-1_mismatch_ | AUAUCUCAACAAGAAUCAUC |
| | A-C | |
| | 20_6-6_internal_ | |
| | loop-symmetric_ | |
| | UGGAUC-UGCAGU | |
| 624 | -8_6-6_internal_ | GAUAUUGUGACAUAUCUCUGCACUCAU |
| | loop-symmetric_ | CACACAAAAGAUGCAAAUCUUCUAUAG |
| | AGUUCA-AUCGUA | ACUGAUCAGGGAGGGGGCAUUCUAAU |
| | 4_1-1_mismatch_ | AUAUCUCAUCGUAAAUCAUC |
| | A-C | |
| | 20_6-6_internal_ | |
| | loop-symmetric_ | |
| | UGGAUC-ACUGAU | |
| 625 | -8_6-6_internal_ | GAUAUUGUGACAUAUCUCUGCACUCAU |
| | loop-symmetric_ | CACACAAAAGAUGCAAAUCUUCUAUAG |
| | AGUUCA-CAGUUC | AAGUGUCAGGGAGGGGGCAUCUUAAU |
| | 5_1-1_mismatch_ | AUAUCUCCAGUUCAAUCAUC |
| | A-C | |
| | 20_6-6_internal_ | |
| | loop-symmetric_ | |
| | UGGAUC-AAGUGU | |
| 626 | -8_6-6_internal_ | GAUAUUGUGACAUAUCUCUGCACUCAU |
| | loop-symmetric_ | CACACAAAAGAUGCAAAUCUUCUAUAG |
| | AGUUCA-CUCUGG | CCGGAUCAGGGAGGGGGCAUUUUAAU |
| | 20_6-6_internal_ | AUAUCUCCUCUGGAAUCAUC |
| | loop-symmetric_ | |
| | UGGAUC-CCGGAU | |
| 627 | -8_6-6_internal_ | GAUAUUGUGACAUAUCUCUGCACUCAU |
| | loop-symmetric_ | CACACAAAAGAUGCAAAUCUUCUAUAA |
| | AGUUCA-CCUCAA | UCAAAACAGGGAGGGGGCAUUUUAAC |
| | 0_1-1_mismatch_ | AUAUCUCCCUCAAAAUCAUC |
| | A-C | |
| | 21_6-6_internal_ | |
| | loop-symmetric_ | |
| | GGAUCC-AUCAAA | |
| 628 | -8_6-6_internal_ | GAUAUUGUGACAUAUCUCUGCACUCAU |
| | loop-symmetric_ | CACACAAAAGAUGCAAAUCUUCUAUAA |
| | AGUUCA-GCCUGG | CUGCAACAGGGAGGGGGCAUUUCAAU |
| | 3_1-1_mismatch_ | AUAUCUCGCCUGGAAUCAUC |
| | A-C | |
| | 21_6-6_internal_ | |
| | loop-symmetric_ | |
| | GGAUCC-ACUGCA | |
| 629 | -8_6-6_internal_ | GAUAUUGUGACAUAUCUCUGCACUCAU |
| | loop-symmetric_ | CACACAAAAGAUGCAAAUCUUCUAUAA |
| | AGUUCA-GUUUAA | AGUGGACAGGGAGGGGGCAUUCUAAU |
| | 4_1-1_mismatch_ | AUAUCUCGUUUAAAAUCAUC |
| | A-C | |
| | 21_6-6_internal_ | |
| | loop-symmetric_ | |
| | GGAUCC-AAGUGG | |
| 630 | -8_6-6_internal_ | GAUAUUGUGACAUAUCUCUGCACUCAU |
| | loop-symmetric_ | CACACAAAAGAUGCAAAUCUUCUAUAU |
| | AGUUCA-GCAGGA | UUAUAACAGGGAGGGGGCAUCUUAAU |
| | 5_1-1_mismatch_ | AUAUCUCGCAGGAAAUCAUC |
| | A-C | |
| | 21_6-6_internal_ | |
| | loop-symmetric_ | |
| | GGAUCC-UUUAUA | |
| 631 | -8_6-6_internal_ | GAUAUUGUGACAUAUCUCUGCACUCAU |
| | loop-symmetric_ | CACACAAAAGAUGCAAAUCUUCUAUAU |
| | AGUUCA-CCCCGG | UGCGAACAGGGAGGGGGCAUUUUAAU |
| | 21_6-6_internal_ | AUAUCUCCCCCGGAAUCAUC |
| | loop-symmetric_ | |
| | GGAUCC-UUGCGA | |
| 632 | -8_6-6_internal_ | GAUAUUGUGACAUAUCUCUGCACUCAU |
| | loop-symmetric_ | CACACAAAAGAUGCAAAUCUUCUAUUA |
| | AGUUCA-GGUCCG | AAAACACAGGGAGGGGGCAUUUUAAC |
| | 0_1-1_mismatch_ | AUAUCUCGGUCCGAAUCAUC |
| | A-C | |
| | 22_6-6_internal_ | |
| | loop-symmetric_ | |
| | GAUCCU-UAAAAA | |
| 633 | -8_6-6_internal_ | GAUAUUGUGACAUAUCUCUGCACUCAU |
| | loop-symmetric_ | CACACAAAAGAUGCAAAUCUUCUAUUA |
| | AGUUCA-CCGCAG | CACGCACAGGGAGGGGGCAUUUCAAUA |
| | 3_1-1_mismatch_ | UAUCUCCCGCAGAAUCAUC |
| | A-C | |
| | 22_6-6_internal_ | |
| | loop-symmetric_ | |
| | GAUCCU-UACACG | |
| 634 | -8_6-6_internal_ | GAUAUUGUGACAUAUCUCUGCACUCAU |
| | loop-symmetric_ | CACACAAAAGAUGCAAAUCUUCUAUCU |
| | AGUUCA-GGUUUC | AUAGCACAGGGAGGGGGCAUUCUAAU |
| | 4_1-1_mismatch_ | AUAUCUCGGUUUCAAUCAUC |
| | A-C | |
| | 22_6-6_internal_ | |
| | loop-symmetric_ | |
| | GAUCCU-CUAUAG | |
| 635 | -8_6-6_internal_ | GAUAUUGUGACAUAUCUCUGCACUCAU |
| | loop-symmetric_ | CACACAAAAGAUGCAAAUCUUCUAUCC |
| | AGUUCA-GAUUGA | CUGGCACAGGGAGGGGGCAUCUUAAU |
| | 5_1-1_mismatch_ | AUAUCUCGAUUGAAAUCAUC |
| | A-C | |
| | 22_6-6_internal_ | |
| | loop-symmetric_ | |
| | GAUCCU-CCCUGG | |
| 636 | -8_6-6_internal_ | GAUAUUGUGACAUAUCUCUGCACUCAU |
| | loop-symmetric_ | CACACAAAAGAUGCAAAUCUUCUAUUC |
| | AGUUCA-AUUUAG | CUUACACAGGGAGGGGGCAUUUUAAU |
| | 22_6-6_internal_ | AUAUCUCAUUUAGAAUCAUC |
| | loop-symmetric_ | |
| | GAUCCU-UCCUUA | |
| 637 | -8_6-6_internal_ | GAUAUUGUGACAUAUCUCUGCACUCAU |
| | loop-symmetric_ | CACACAAAAGAUGCAAAUCUUCUAAAC |
| | AGUUCA-GCGUUA | GUACCACAGGGAGGGGGCAUUUUAAC |
| | 0_1-1_mismatch_ | AUAUCUCGCGUUAAAUCAUC |
| | A-C | |
| | 23_6-6_internal_ | |
| | loop-symmetric_ | |
| | AUCCUA-AACGUA | |
| 638 | -8_6-6_internal_ | GAUAUUGUGACAUAUCUCUGCACUCAU |
| | loop-symmetric_ | CACACAAAAGAUGCAAAUCUUCUACAA |
| | AGUUCA-CCGCCC | AAACCACAGGGAGGGGGCAUUUCAAU |
| | 3_1-1_mismatch_ | AUAUCUCCCGCCCAAUCAUC |
| | A-C | |
| | 23_6-6_internal_ | |
| | loop-symmetric_ | |
| | AUCCUA-CAAAAA | |
| 639 | -8_6-6_internal_ | GAUAUUGUGACAUAUCUCUGCACUCAU |
| | loop-symmetric_ | CACACAAAAGAUGCAAAUCUUCUACUC |
| | AGUUCA-GCCCGA | GCGCCACAGGGAGGGGGCAUUCUAAUA |
| | 4_1-1_mismatch_ | UAUCUCGCCCGAAAUCAUC |
| | A-C | |
| | 23_6-6_internal_ | |
| | loop-symmetric_ | |
| | AUCCUA-CUCGCG | |
| 640 | -8_6-6_internal_ | GAUAUUGUGACAUAUCUCUGCACUCAU |
| | loop-symmetric_ | CACACAAAAGAUGCAAAUCUUCUAAUG |
| | AGUUCA-GUUUUA | CACCCACAGGGAGGGGGCAUCUUAAUA |
| | 5_1-1_mismatch_ | UAUCUCGUUUUAAAUCAUC |
| | A-C | |
| | 23_6-6_internal_ | |
| | loop-symmetric_ | |
| | AUCCUA-AUGCAC | |
| 641 | -8_6-6_internal_ | GAUAUUGUGACAUAUCUCUGCACUCAU |
| | loop-symmetric_ | CACACAAAAGAUGCAAAUCUUCUACAU |
| | AGUUCA-AUACCG | UCCCCACAGGGAGGGGGCAUUUUAAUA |
| | 23_6-6_internal_ | UAUCUCAUACCGAAUCAUC |
| | loop-symmetric_ | |
| | AUCCUA-CAUUCC | |
| 642 | -8_6-6_internal_ | GAUAUUGUGACAUAUCUCUGCACUCAU |
| | loop-symmetric_ | CACACAAAAGAUGCAAAUCUUCUCCUU |
| | AGUUCA-AUUGUA | UUUCCACAGGGAGGGGGCAUUUUAAC |
| | 0_1-1_mismatch_ | AUAUCUCAUUGUAAAUCAUC |
| | A-C | |
| | 24_6-6_internal_ | |
| | loop-symmetric_ | |
| | UCCUAU-CCUUUU | |
| 643 | -8_6-6_internal_ | GAUAUUGUGACAUAUCUCUGCACUCAU |
| | loop-symmetric_ | CACACAAAAGAUGCAAAUCUUCUUGCA |
| | AGUUCA-CUAUGG | UUUCCACAGGGAGGGGGCAUUUCAAU |
| | 3_1-1_mismatch_ | AUAUCUCCUAUGGAAUCAUC |
| | A-C | |
| | 24_6-6_internal_ | |
| | loop-symmetric_ | |
| | UCCUAU-UGCAUU | |
| 644 | -8_6-6_internal_ | GAUAUUGUGACAUAUCUCUGCACUCAU |
| | loop-symmetric_ | CACACAAAAGAUGCAAAUCUUCUCCUC |
| | AGUUCA-GCGUUG | CUUCCACAGGGAGGGGGCAUUCUAAUA |
| | 4_1-1_mismatch_ | UAUCUCGCGUUGAAUCAUC |
| | A-C | |
| | 24_6-6_internal_ | |
| | loop-symmetric_ | |
| | UCCUAU-CCUCCU | |
| 645 | -8_6-6_internal_ | GAUAUUGUGACAUAUCUCUGCACUCAU |
| | loop-symmetric_ | CACACAAAAGAUGCAAAUCUUCUCUCU |
| | AGUUCA-GACGAA | ACUCCACAGGGAGGGGGCAUCUUAAUA |
| | 5_1-1_mismatch_ | UAUCUCGACGAAAAUCAUC |
| | A-C | |
| | 24_6-6_internal_ | |
| | loop-symmetric_ | |
| | UCCUAU-CUCUAC | |
| 646 | -8_6-6_internal_ | GAUAUUGUGACAUAUCUCUGCACUCAU |
| | loop-symmetric_ | CACACAAAAGAUGCAAAUCUUCUCCCU |
| | AGUUCA-CUAUGG | UUUCCACAGGGAGGGGGCAUUUUAAU |
| | 24_6-6_internal_ | AUAUCUCCUAUGGAAUCAUC |
| | loop-symmetric_ | |
| | UCCUAU-CCCUUU | |
| 647 | -8_6-6_internal_ | GAUAUUGUGACAUAUCUCUGCACUCAU |
| | loop-symmetric_ | CACACAAAAGAUGCAAAUCUUCAAUCU |
| | AGUUCA-CCGUCG | UAUCCACAGGGAGGGGGCAUUUUAAC |
| | 0_1-1_mismatch_ | AUAUCUCCCGUCGAAUCAUC |
| | A-C | |
| | 25_6-6_internal_ | |
| | loop-symmetric_ | |
| | CCUAUA-AAUCUU | |
| 648 | -8_6-6_internal_ | GAUAUUGUGACAUAUCUCUGCACUCAU |
| | loop-symmetric_ | CACACAAAAGAUGCAAAUCUUCCUCCG |
| | AGUUCA-GUAUGG | CAUCCACAGGGAGGGGGCAUUUCAAUA |
| | 3_1-1_mismatch_ | UAUCUCGUAUGGAAUCAUC |
| | A-C | |
| | 25_6-6_internal_ | |
| | loop-symmetric_ | |
| | CCUAUA-CUCCGC | |
| 649 | -8_6-6_internal_ | GAUAUUGUGACAUAUCUCUGCACUCAU |
| | loop-symmetric_ | CACACAAAAGAUGCAAAUCUUCGAUUC |
| | AGUUCA-CAUUUC | AAUCCACAGGGAGGGGGCAUUCUAAU |
| | 4_1-1_mismatch_ | AUAUCUCCAUUUCAAUCAUC |
| | A-C | |
| | 25_6-6_internal_ | |
| | loop-symmetric_ | |
| | CCUAUA-GAUUCA | |
| 650 | -8_6-6_internal_ | GAUAUUGUGACAUAUCUCUGCACUCAU |
| | loop-symmetric_ | CACACAAAAGAUGCAAAUCUUCGUCGC |
| | AGUUCA-CUUUCG | UAUCCACAGGGAGGGGGCAUCUUAAU |
| | 5_1-1_mismatch_ | AUAUCUCCUUUCGAAUCAUC |
| | A-C | |
| | 25_6-6_internal_ | |
| | loop-symmetric_ | |
| | CCUAUA-GUCGCU | |
| 651 | -8_6-6_internal_ | GAUAUUGUGACAUAUCUCUGCACUCAU |
| | loop-symmetric_ | CACACAAAAGAUGCAAAUCUUCCGCAU |
| | AGUUCA-ACUGUA | CAUCCACAGGGAGGGGGCAUUUUAAU |
| | 25_6-6_internal_ | AUAUCUCACUGUAAAUCAUC |
| | loop-symmetric_ | |
| | CCUAUA-CGCAUC | |
| 652 | -8_6-6_internal_ | GAUAUUGUGACAUAUCUCUGCACUCAU |
| | loop-symmetric_ | CACACAAAAGAUGCAAAUCUUACCUGC |
| | AGUUCA-AUUUAA | GAUCCACAGGGAGGGGGCAUUUCAAU |
| | 3_1-1_mismatch_ | AUAUCUCAUUUAAAAUCAUC |
| | A-C | |
| | 26_6-6_internal_ | |
| | loop-symmetric_ | |
| | CUAUAG-ACCUGC | |
| 653 | -8_6-6_internal_ | GAUAUUGUGACAUAUCUCUGCACUCAU |
| | loop-symmetric_ | CACACAAAAGAUGCAAAUCUUACGGCC |
| | AGUUCA-AUUCCC | GAUCCACAGGGAGGGGGCAUUCUAAU |
| | 4_1-1_mismatch_ | AUAUCUCAUUCCCAAUCAUC |
| | A-C | |
| | 26_6-6_internal_ | |
| | loop-symmetric_ | |
| | CUAUAG-ACGGCC | |
| 654 | -8_6-6_internal_ | GAUAUUGUGACAUAUCUCUGCACUCAU |
| | loop-symmetric_ | CACACAAAAGAUGCAAAUCUUGGUAUC |
| | AGUUCA-GUCGGG | GAUCCACAGGGAGGGGGCAUCUUAAU |
| | 5_1-1_mismatch_ | AUAUCUCGUCGGGAAUCAUC |
| | A-C | |
| | 26_6-6_internal_ | |
| | loop-symmetric_ | |
| | CUAUAG-GGUAUC | |
| 655 | -8_6-6_internal_ | GAUAUUGUGACAUAUCUCUGCACUCAU |
| | loop-symmetric_ | CACACAAAAGAUGCAAAUCUUAACGCU |
| | AGUUCA-AACCGG | GAUCCACAGGGAGGGGGCAUUUUAAU |
| | 26_6-6_internal_ | AUAUCUCAACCGGAAUCAUC |
| | loop-symmetric_ | |
| | CUAUAG-AACGCU | |
| 656 | -8_6-6_internal_ | GAUAUUGUGACAUAUCUCUGCACUCAU |
| | loop-symmetric_ | CACACAAAAGAUGCAAAUCUCAGGACG |
| | AGUUCA-ACGUUG | GAUCCACAGGGAGGGGGCAUUUUAAC |
| | 0_1-1_mismatch_ | AUAUCUCACGUUGAAUCAUC |
| | A-C | |
| | 27_6-6_internal_ | |
| | loop-symmetric_ | |
| | UAUAGA-CAGGAC | |
| 657 | -8_6-6_internal_ | GAUAUUGUGACAUAUCUCUGCACUCAU |
| | loop-symmetric_ | CACACAAAAGAUGCAAAUCUAUAAGCG |
| | AGUUCA-AUUUUC | GAUCCACAGGGAGGGGGCAUUUCAAU |
| | 3_1-1_mismatch_ | AUAUCUCAUUUUCAAUCAUC |
| | A-C | |
| | 27_6-6_internal_ | |
| | loop-symmetric_ | |
| | UAUAGA-AUAAGC | |
| 658 | -8_6-6_internal_ | GAUAUUGUGACAUAUCUCUGCACUCAU |
| | loop-symmetric_ | CACACAAAAGAUGCAAAUCUCAUCGUG |
| | AGUUCA-CCGUAG | GAUCCACAGGGAGGGGGCAUCUUAAU |
| | 5_1-1_mismatch_ | AUAUCUCCCGUAGAAUCAUC |
| | A-C | |
| | 27_6-6_internal_ | |
| | loop-symmetric_ | |
| | UAUAGA-CAUCGU | |
| 659 | -8_6-6_internal_ | GAUAUUGUGACAUAUCUCUGCACUCAU |
| | loop-symmetric_ | CACACAAAAGAUGCAAAUCUAAUUACG |
| | AGUUCA-CAUCGA | GAUCCACAGGGAGGGGGCAUUUUAAU |
| | 27_6-6_internal_ | AUAUCUCCAUCGAAAUCAUC |
| | loop-symmetric_ | |
| | UAUAGA-AAUUAC | |
| 660 | -8_6-6_internal_ | GAUAUUGUGACAUAUCUCUGCACUCAU |
| | loop-symmetric_ | CACACAAAAGAUGCAAAUCGGAUUAA |
| | AGUUCA-AUCCCA | GGAUCCACAGGGAGGGGGCAUUUUAA |
| | 0_1-1_mismatch_ | CAUAUCUCAUCCCAAAUCAUC |
| | A-C | |
| | 28_6-6_internal_ | |
| | loop-symmetric_ | |
| | AUAGAA-GGAUUA | |
| 661 | -8_6-6_internal_ | GAUAUUGUGACAUAUCUCUGCACUCAU |
| | loop-symmetric_ | CACACAAAAGAUGCAAAUCCCGCGCAG |
| | AGUUCA-GCUCGA | GAUCCACAGGGAGGGGGCAUUUCAAU |
| | 3_1-1_mismatch_ | AUAUCUCGCUCGAAAUCAUC |
| | A-C | |
| | 28_6-6_internal_ | |
| | loop-symmetric_ | |
| | AUAGAA-CCGCGC | |
| 662 | -8_6-6_internal_ | GAUAUUGUGACAUAUCUCUGCACUCAU |
| | loop-symmetric_ | CACACAAAAGAUGCAAAUCCUGGAGAG |
| | AGUUCA-CUUUUA | GAUCCACAGGGAGGGGGCAUUCUAAU |
| | 4_1-1_mismatch_ | AUAUCUCCUUUUAAAUCAUC |
| | A-C | |
| | 28_6-6_internal_ | |
| | loop-symmetric_ | |
| | AUAGAA-CUGGAG | |
| 663 | -8_6-6_internal_ | GAUAUUGUGACAUAUCUCUGCACUCAU |
| | loop-symmetric_ | CACACAAAAGAUGCAAAUCCCAGUCAG |
| | AGUUCA-CUUGGG | GAUCCACAGGGAGGGGGCAUCUUAAU |
| | 5_1-1_mismatch_ | AUAUCUCCUUGGGAAUCAUC |
| | A-C | |
| | 28_6-6_internal_ | |
| | loop-symmetric_ | |
| | AUAGAA-CCAGUC | |
| 664 | -8_6-6_internal_ | GAUAUUGUGACAUAUCUCUGCACUCAU |
| | loop-symmetric_ | CACACAAAAGAUGCAAAUCGGGAACAG |
| | AGUUCA-AACCCG | GAUCCACAGGGAGGGGGCAUUUUAAU |
| | 28_6-6_internal_ | AUAUCUCAACCCGAAUCAUC |
| | loop-symmetric_ | |
| | AUAGAA-GGGAAC | |
| 665 | -8_6-6_internal_ | GAUAUUGUGACAUAUCUCUGCACUCAU |
| | loop-symmetric_ | CACACAAAAGAUGCAAAUACCUUUUAG |
| | AGUUCA-ACUCAG | GAUCCACAGGGAGGGGGCAUUUUAAC |
| | 0_1-1_mismatch_ | AUAUCUCACUCAGAAUCAUC |
| | A-C | |
| | 29_6-6_internal_ | |
| | loop-symmetric_ | |
| | UAGAAG-ACCUUU | |
| 666 | -8_6-6_internal_ | GAUAUUGUGACAUAUCUCUGCACUCAU |
| | loop-symmetric_ | CACACAAAAGAUGCAAAUAGUAUUUA |
| | AGUUCA-AAGUAA | GGAUCCACAGGGAGGGGGCAUUUCAA |
| | 3_1-1_mismatch_ | UAUAUCUCAAGUAAAAUCAUC |
| | A-C | |
| | 29_6-6_internal_ | |
| | loop-symmetric_ | |
| | UAGAAG-AGUAUU | |
| 667 | -8_6-6_internal_ | GAUAUUGUGACAUAUCUCUGCACUCAU |
| | loop-symmetric_ | CACACAAAAGAUGCAAAUGGACACUAG |
| | AGUUCA-AAUCGA | GAUCCACAGGGAGGGGGCAUUCUAAU |
| | 4_1-1_mismatch_ | AUAUCUCAAUCGAAAUCAUC |
| | A-C | |
| | 29_6-6_internal_ | |
| | loop-symmetric_ | |
| | UAGAAG-GGACAC | |
| 668 | -8_6-6_internal_ | GAUAUUGUGACAUAUCUCUGCACUCAU |
| | loop-symmetric_ | CACACAAAAGAUGCAAAUAAGAUUUA |
| | AGUUCA-AAGUUC | GGAUCCACAGGGAGGGGGCAUCUUAA |
| | 5_1-1_mismatch_ | UAUAUCUCAAGUUCAAUCAUC |
| | A-C | |
| | 29_6-6_internal_ | |
| | loop-symmetric_ | |
| | UAGAAG-AAGAUU | |
| 669 | -8_6-6_internal_ | GAUAUUGUGACAUAUCUCUGCACUCAU |
| | loop-symmetric_ | CACACAAAAGAUGCAAAUAUAGUUUA |
| | AGUUCA-GAUGUC | GGAUCCACAGGGAGGGGGCAUUUUAA |
| | 29_6-6_internal_ | UAUAUCUCGAUGUCAAUCAUC |
| | loop-symmetric_ | |
| | UAGAAG-AUAGUU | |
| 670 | -8_6-6_internal_ | GAUAUUGUGACAUAUCUCUGCACUCAU |
| | loop-symmetric_ | CACACAAAAGAUGCAAACAGGACAUAG |
| | AGUUCA-CCCUCG | GAUCCACAGGGAGGGGGCAUUUUAAC |
| | 0_1-1_mismatch_ | AUAUCUCCCCUCGAAUCAUC |
| | A-C | |
| | 30_6-6_internal_ | |
| | loop-symmetric_ | |
| | AGAAGA-CAGGAC | |
| 671 | -8_6-6_internal_ | GAUAUUGUGACAUAUCUCUGCACUCAU |
| | loop-symmetric_ | CACACAAAAGAUGCAAACAGGUGAUA |
| | AGUUCA-GGUUUC | GGAUCCACAGGGAGGGGGCAUUCUAA |
| | 4_1-1_mismatch_ | UAUAUCUCGGUUUCAAUCAUC |
| | A-C | |
| | 30_6-6_internal_ | |
| | loop-symmetric_ | |
| | AGAAGA-CAGGUG | |
| 672 | -8_6-6_internal_ | GAUAUUGUGACAUAUCUCUGCACUCAU |
| | loop-symmetric_ | CACACAAAAGAUGCAAACAACCCAUAG |
| | AGUUCA-CACCAA | GAUCCACAGGGAGGGGGCAUCUUAAU |
| | 5_1-1_mismatch_ | AUAUCUCCACCAAAAUCAUC |
| | A-C | |
| | 30_6-6_internal_ | |
| | loop-symmetric_ | |
| | AGAAGA-CAACCC | |
| 673 | -8_6-6_internal_ | GAUAUUGUGACAUAUCUCUGCACUCAU |
| | loop-symmetric_ | CACACAAAAGAUGCAAAGGGUAAAUA |
| | AGUUCA-ACCAUG | GGAUCCACAGGGAGGGGGCAUUUUAA |
| | 30_6-6_internal_ | UAUAUCUCACCAUGAAUCAUC |
| | loop-symmetric_ | |
| | AGAAGA-GGGUAA | |
| 674 | -8_6-6_internal_ | GAUAUUGUGACAUAUCUCUGCACUCAU |
| | loop-symmetric_ | CACACAAAAGAUGCAAUGUGAGUAUA |
| | AGUUCA-GCAUUC | GGAUCCACAGGGAGGGGGCAUUUUAA |
| | 0_1-1_mismatch_ | CAUAUCUCGCAUUCAAUCAUC |
| | A-C | |
| | 31_6-6_internal_ | |
| | loop-symmetric_ | |
| | GAAGAU-UGUGAG | |
| 675 | -8_6-6_internal_ | GAUAUUGUGACAUAUCUCUGCACUCAU |
| | loop-symmetric_ | CACACAAAAGAUGCAACCUAGGUAUAG |
| | AGUUCA-AAAUCG | GAUCCACAGGGAGGGGGCAUUUCAAU |
| | 3_1-1_mismatch_ | AUAUCUCAAAUCGAAUCAUC |
| | A-C | |
| | 31_6-6_internal_ | |
| | loop-symmetric_ | |
| | GAAGAU-CCUAGG | |
| 676 | -8_6-6_internal_ | GAUAUUGUGACAUAUCUCUGCACUCAU |
| | loop-symmetric_ | CACACAAAAGAUGCAAUCACAGUAUAG |
| | AGUUCA-AUAUUA | GAUCCACAGGGAGGGGGCAUUCUAAU |
| | 4_1-1_mismatch_ | AUAUCUCAUAUUAAAUCAUC |
| | A-C | |
| | 31_6-6_internal_ | |
| | loop-symmetric_ | |
| | GAAGAU-UCACAG | |
| 677 | -8_6-6_internal_ | GAUAUUGUGACAUAUCUCUGCACUCAU |
| | loop-symmetric_ | CACACAAAAGAUGCAACCUAAGUAUAG |
| | AGUUCA-GGUGUA | GAUCCACAGGGAGGGGGCAUCUUAAU |
| | 5_1-1_mismatch_ | AUAUCUCGGUGUAAAUCAUC |
| | A-C | |
| | 31_6-6_internal_ | |
| | loop-symmetric_ | |
| | GAAGAU-CCUAAG | |
| 678 | -8_6-6_internal_ | GAUAUUGUGACAUAUCUCUGCACUCAU |
| | loop-symmetric_ | CACACAAAAGAUGCAACGAAGGUAUA |
| | AGUUCA-CCCUAG | GGAUCCACAGGGAGGGGGCAUUUUAA |
| | 31_6-6_internal_ | UAUAUCUCCCCUAGAAUCAUC |
| | loop-symmetric_ | |
| | GAAGAU-CGAAGG | |
| 679 | -8_6-6_internal_ | GAUAUUGUGACAUAUCUCUGCACUCAU |
| | loop-symmetric_ | CACACAAAAGAUGCACUAACCCUAUAG |
| | AGUUCA-ACCUAG | GAUCCACAGGGAGGGGGCAUUUUAAC |
| | 0_1-1_mismatch_ | AUAUCUCACCUAGAAUCAUC |
| | A-C | |
| | 32_6-6_internal_ | |
| | loop-symmetric_ | |
| | AAGAUU-CUAACC | |
| 680 | -8_6-6_internal_ | GAUAUUGUGACAUAUCUCUGCACUCAU |
| | loop-symmetric_ | CACACAAAAGAUGCACUGGACCUAUAG |
| | AGUUCA-ACCCGA | GAUCCACAGGGAGGGGGCAUUUCAAU |
| | 3_1-1_mismatch_ | AUAUCUCACCCGAAAUCAUC |
| | A-C | |
| | 32_6-6_internal_ | |
| | loop-symmetric_ | |
| | AAGAUU-CUGGAC | |
| 681 | -8_6-6_internal_ | GAUAUUGUGACAUAUCUCUGCACUCAU |
| | loop-symmetric_ | CACACAAAAGAUGCACUCAGGCUAUAG |
| | AGUUCA-GCUCGA | GAUCCACAGGGAGGGGGCAUCUUAAU |
| | 5_1-1_mismatch_ | AUAUCUCGCUCGAAAUCAUC |
| | A-C | |
| | 32_6-6_internal_ | |
| | loop-symmetric_ | |
| | AAGAUU-CUCAGG | |
| 682 | -8_6-6_internal_ | GAUAUUGUGACAUAUCUCUGCACUCAU |
| | loop-symmetric_ | CACACAAAAGAUGCACUAAGACUAUAG |
| | AGUUCA-ACCUUC | GAUCCACAGGGAGGGGGCAUUUUAAU |
| | 32_6-6_internal_ | AUAUCUCACCUUCAAUCAUC |
| | loop-symmetric_ | |
| | AAGAUU-CUAAGA | |
| 683 | -8_6-6_internal_ | GAUAUUGUGACAUAUCUCUGCACUCAU |
| | loop-symmetric_ | CACACAAAAGAUGCUCACGAUCUAUAG |
| | AGUUCA-AAUGUG | GAUCCACAGGGAGGGGGCAUUUUAAC |
| | 0_1-1_mismatch_ | AUAUCUCAAUGUGAAUCAUC |
| | A-C | |
| | 33_6-6_internal_ | |
| | loop-symmetric_ | |
| | AGAUUU-UCACGA | |
| 684 | -8_6-6_internal_ | GAUAUUGUGACAUAUCUCUGCACUCAU |
| | loop-symmetric_ | CACACAAAAGAUGCCCACUGUCUAUAG |
| | AGUUCA-CACAGA | GAUCCACAGGGAGGGGGCAUUUCAAU |
| | 3_1-1_mismatch_ | AUAUCUCCACAGAAAUCAUC |
| | A-C | |
| | 33_6-6_internal_ | |
| | loop-symmetric_ | |
| | AGAUUU-CCACUG | |
| 685 | -8_6-6_internal_ | GAUAUUGUGACAUAUCUCUGCACUCAU |
| | loop-symmetric_ | CACACAAAAGAUGCUACCUGUCUAUAG |
| | AGUUCA-CACGAG | GAUCCACAGGGAGGGGGCAUUCUAAU |
| | 4_1-1_mismatch_ | AUAUCUCCACGAGAAUCAUC |
| | A-C | |
| | 33_6-6_internal_ | |
| | loop-symmetric_ | |
| | AGAUUU-UACCUG | |
| 686 | -8_6-6_internal_ | GAUAUUGUGACAUAUCUCUGCACUCAU |
| | loop-symmetric_ | CACACAAAAGAUGCUACUAAUCUAUAG |
| | AGUUCA-GCAUGA | GAUCCACAGGGAGGGGGCAUCUUAAU |
| | 5_1-1_mismatch_ | AUAUCUCGCAUGAAAUCAUC |
| | A-C | |
| | 33_6-6_internal_ | |
| | loop-symmetric_ | |
| | AGAUUU-UACUAA | |
| 687 | -8_6-6_internal_ | GAUAUUGUGACAUAUCUCUGCACUCAU |
| | loop-symmetric_ | CACACAAAAGAUGCUUCGGGUCUAUAG |
| | AGUUCA-ACAUGG | GAUCCACAGGGAGGGGGCAUUUUAAU |
| | 33_6-6_internal_ | AUAUCUCACAUGGAAUCAUC |
| | loop-symmetric_ | |
| | AGAUUU-UUCGGG | |
| 688 | -8_6-6_internal_ | GAUAUUGUGACAUAUCUCUGCACUCAU |
| | loop-symmetric_ | CACACAAAAGAUGGCUUGAUUCUAUA |
| | AGUUCA-CUUCAG | GGAUCCACAGGGAGGGGGCAUUUUAA |
| | 0_1-1_mismatch_ | CAUAUCUCCUUCAGAAUCAUC |
| | A-C | |
| | 34_6-6_internal_ | |
| | loop-symmetric_ | |
| | GAUUUG-GCUUGA | |
| 689 | -8_6-6_internal_ | GAUAUUGUGACAUAUCUCUGCACUCAU |
| | loop-symmetric_ | CACACAAAAGAUGAAUAAAUUCUAUA |
| | AGUUCA-ACUCGA | GGAUCCACAGGGAGGGGGCAUUUCAA |
| | 3_1-1_mismatch_ | UAUAUCUCACUCGAAAUCAUC |
| | A-C | |
| | 34_6-6_internal_ | |
| | loop-symmetric_ | |
| | GAUUUG-AAUAAA | |
| 690 | -8_6-6_internal_ | GAUAUUGUGACAUAUCUCUGCACUCAU |
| | loop-symmetric_ | CACACAAAAGAUGACCCAAUUCUAUAG |
| | AGUUCA-GAUUCG | GAUCCACAGGGAGGGGGCAUUCUAAU |
| | 4_1-1_mismatch_ | AUAUCUCGAUUCGAAUCAUC |
| | A-C | |
| | 34_6-6_internal_ | |
| | loop-symmetric_ | |
| | GAUUUG-ACCCAA | |
| 691 | -8_6-6_internal_ | GAUAUUGUGACAUAUCUCUGCACUCAU |
| | loop-symmetric_ | CACACAAAAGAUGGCGUUAUUCUAUA |
| | AGUUCA-AUUUAA | GGAUCCACAGGGAGGGGGCAUCUUAA |
| | 5_1-1_mismatch_ | UAUAUCUCAUUUAAAAUCAUC |
| | A-C | |
| | 34_6-6_internal_ | |
| | loop-symmetric_ | |
| | GAUUUG-GCGUUA | |
| 692 | -8_6-6_internal_ | GAUAUUGUGACAUAUCUCUGCACUCAU |
| | loop-symmetric_ | CACACAAAAGAUGAAUGAAUUCUAUA |
| | AGUUCA-GCAUGG | GGAUCCACAGGGAGGGGGCAUUUUAA |
| | 34_6-6_internal_ | UAUAUCUCGCAUGGAAUCAUC |
| | loop-symmetric_ | |
| | GAUUUG-AAUGAA | |
| 693 | -8_6-6_internal_ | GAUAUUGUGACAUAUCUCUGCACUCAU |
| | loop-symmetric_ | CACACAAAAGAUUUUCCACUUCUAUAG |
| | AGUUCA-CACCAA | GAUCCACAGGGAGGGGGCAUUUUAAC |
| | 0_1-1_mismatch_ | AUAUCUCCACCAAAAUCAUC |
| | A-C | |
| | 35_6-6_internal_ | |
| | loop-symmetric_ | |
| | AUUUGC-UUUCCA | |
| 694 | -8_6-6_internal_ | GAUAUUGUGACAUAUCUCUGCACUCAU |
| | loop-symmetric_ | CACACAAAAGAUCUUCCCCUUCUAUAG |
| | AGUUCA-ACUCGA | GAUCCACAGGGAGGGGGCAUUUCAAU |
| | 3_1-1_mismatch_ | AUAUCUCACUCGAAAUCAUC |
| | A-C | |
| | 35_6-6_internal_ | |
| | loop-symmetric_ | |
| | AUUUGC-CUUCCC | |
| 695 | -8_6-6_internal_ | GAUAUUGUGACAUAUCUCUGCACUCAU |
| | loop-symmetric_ | CACACAAAAGAUCCCUAGCUUCUAUAG |
| | AGUUCA-AUCGGG | GAUCCACAGGGAGGGGGCAUUCUAAU |
| | 4_1-1_mismatch_ | AUAUCUCAUCGGGAAUCAUC |
| | A-C | |
| | 35_6-6_internal_ | |
| | loop-symmetric_ | |
| | AUUUGC-CCCUAG | |
| 696 | -8_6-6_internal_ | GAUAUUGUGACAUAUCUCUGCACUCAU |
| | loop-symmetric_ | CACACAAAAGAUUUCGUGCUUCUAUAG |
| | AGUUCA-ACUUUC | GAUCCACAGGGAGGGGGCAUCUUAAU |
| | 5_1-1_mismatch_ | AUAUCUCACUUUCAAUCAUC |
| | A-C | |
| | 35_6-6_internal_ | |
| | loop-symmetric_ | |
| | AUUUGC-UUCGUG | |
| 697 | -8_6-6_internal_ | GAUAUUGUGACAUAUCUCUGCACUCAU |
| | loop-symmetric_ | CACACAAAAGAUAGGUCGCUUCUAUAG |
| | AGUUCA-CCUCCG | GAUCCACAGGGAGGGGGCAUUUUAAU |
| | 35_6-6_internal_ | AUAUCUCCCUCCGAAUCAUC |
| | loop-symmetric_ | |
| | AUUUGC-AGGUCG | |
| 698 | -8_6-6_internal_ | GAUAUUGUGACAUAUCUCUGCACUCAU |
| | loop-symmetric_ | CACACAAAAGACAGGUUUCUUCUAUAG |
| | AGUUCA-GCUUAA | GAUCCACAGGGAGGGGGCAUUUUAAC |
| | 0_1-1_mismatch_ | AUAUCUCGCUUAAAAUCAUC |
| | A-C | |
| | 36_6-6_internal_ | |
| | loop-symmetric_ | |
| | UUUGCA-CAGGUU | |
| 699 | -8_6-6_internal_ | GAUAUUGUGACAUAUCUCUGCACUCAU |
| | loop-symmetric_ | CACACAAAAGAGAAACUUCUUCUAUAG |
| | AGUUCA-CUUCGA | GAUCCACAGGGAGGGGGCAUUUCAAU |
| | 3_1-1_mismatch_ | AUAUCUCCUUCGAAAUCAUC |
| | A-C | |
| | 36_6-6_internal_ | |
| | loop-symmetric_ | |
| | UUUGCA-GAAACU | |
| 700 | -8_6-6_internal_ | GAUAUUGUGACAUAUCUCUGCACUCAU |
| | loop-symmetric_ | CACACAAAAGAAAAUUUUCUUCUAUA |
| | AGUUCA-CCCUGG | GGAUCCACAGGGAGGGGGCAUUCUAA |
| | 4_1-1_mismatch_ | UAUAUCUCCCCUGGAAUCAUC |
| | A-C | |
| | 36_6-6_internal_ | |
| | loop-symmetric_ | |
| | UUUGCA-AAAUUU | |
| 701 | -8_6-6_internal_ | GAUAUUGUGACAUAUCUCUGCACUCAU |
| | loop-symmetric_ | CACACAAAAGAGUCUUCUCUUCUAUAG |
| | AGUUCA-CUUUUG | GAUCCACAGGGAGGGGGCAUCUUAAU |
| | 5_1-1_mismatch_ | AUAUCUCCUUUUGAAUCAUC |
| | A-C | |
| | 36_6-6_internal_ | |
| | loop-symmetric_ | |
| | UUUGCA-GUCUUC | |
| 702 | -8_6-6_internal_ | GAUAUUGUGACAUAUCUCUGCACUCAU |
| | loop-symmetric_ | CACACAAAAGAACUCUUUCUUCUAUAG |
| | AGUUCA-AUUCCG | GAUCCACAGGGAGGGGGCAUUUUAAU |
| | 36_6-6_internal_ | AUAUCUCAUUCCGAAUCAUC |
| | loop-symmetric_ | |
| | UUUGCA-ACUCUU | |
| 703 | -8_6-6_internal_ | GAUAUUGUGACAUAUCUCUGCACUCAU |
| | loop-symmetric_ | CACACAAAAGUAAGACAUCUUCUAUAG |
| | AGUUCA-CCAUAG | GAUCCACAGGGAGGGGGCAUUUUAAC |
| | 0_1-1_mismatch_ | AUAUCUCCCAUAGAAUCAUC |
| | A-C | |
| | 37_6-6_internal_ | |
| | loop-symmetric_ | |
| | UUGCAU-UAAGAC | |
| 704 | -8_6-6_internal_ | GAUAUUGUGACAUAUCUCUGCACUCAU |
| | loop-symmetric_ | CACACAAAAGCGAUUCAUCUUCUAUAG |
| | AGUUCA-GACCCC | GAUCCACAGGGAGGGGGCAUUUCAAU |
| | 3_1-1_mismatch_ | AUAUCUCGACCCCAAUCAUC |
| | A-C | |
| | 37_6-6_internal_ | |
| | loop-symmetric_ | |
| | UUGCAU-CGAUUC | |
| 705 | -8_6-6_internal_ | GAUAUUGUGACAUAUCUCUGCACUCAU |
| | loop-symmetric_ | CACACAAAAGCGAGUCAUCUUCUAUAG |
| | AGUUCA-GGUCGA | GAUCCACAGGGAGGGGGCAUUCUAAU |
| | 4_1-1_mismatch_ | AUAUCUCGGUCGAAAUCAUC |
| | A-C | |
| | 37_6-6_internal_ | |
| | loop-symmetric_ | |
| | UUGCAU-CGAGUC | |
| 706 | -8_6-6_internal_ | GAUAUUGUGACAUAUCUCUGCACUCAU |
| | loop-symmetric_ | CACACAAAAGCAUAUCAUCUUCUAUAG |
| | AGUUCA-GCCCCA | GAUCCACAGGGAGGGGGCAUCUUAAU |
| | 5_1-1_mismatch_ | AUAUCUCGCCCCAAAUCAUC |
| | A-C | |
| | 37_6-6_internal_ | |
| | loop-symmetric_ | |
| | UUGCAU-CAUAUC | |
| 707 | -8_6-6_internal_ | GAUAUUGUGACAUAUCUCUGCACUCAU |
| | loop-symmetric_ | CACACAAAAGCGUUUCAUCUUCUAUAG |
| | AGUUCA-ACUGUG | GAUCCACAGGGAGGGGGCAUUUUAAU |
| | 37_6-6_internal_ | AUAUCUCACUGUGAAUCAUC |
| | loop-symmetric_ | |
| | UUGCAU-CGUUUC | |
| 708 | -8_6-6_internal_ | GAUAUUGUGACAUAUCUCUGCACUCAU |
| | loop-symmetric_ | CACACAAAAAUCACUAAUCUUCUAUAG |
| | AGUUCA-AUUGGG | GAUCCACAGGGAGGGGGCAUUUUAAC |
| | 0_1-1_mismatch_ | AUAUCUCAUUGGGAAUCAUC |
| | A-C | |
| | 38_6-6_internal_ | |
| | loop-symmetric_ | |
| | UGCAUC-AUCACU | |
| 709 | -8_6-6_internal_ | GAUAUUGUGACAUAUCUCUGCACUCAU |
| | loop-symmetric_ | CACACAAAAUCACUUAAUCUUCUAUAG |
| | AGUUCA-AAGUUA | GAUCCACAGGGAGGGGGCAUUUCAAU |
| | 3_1-1_mismatch_ | AUAUCUCAAGUUAAAUCAUC |
| | A-C | |
| | 38_6-6_internal_ | |
| | loop-symmetric_ | |
| | UGCAUC-UCACUU | |
| 710 | -8_6-6_internal_ | GAUAUUGUGACAUAUCUCUGCACUCAU |
| | loop-symmetric_ | CACACAAAAAAACUUAAUCUUCUAUAG |
| | AGUUCA-CACCAG | GAUCCACAGGGAGGGGGCAUUCUAAU |
| | 4_1-1_mismatch_ | AUAUCUCCACCAGAAUCAUC |
| | A-C | |
| | 38_6-6_internal_ | |
| | loop-symmetric_ | |
| | UGCAUC-AAACUU | |
| 711 | -8_6-6_internal_ | GAUAUUGUGACAUAUCUCUGCACUCAU |
| | loop-symmetric_ | CACACAAAAAUUCUCAAUCUUCUAUAG |
| | AGUUCA-ACAGGG | GAUCCACAGGGAGGGGGCAUCUUAAU |
| | 5_1-1_mismatch_ | AUAUCUCACAGGGAAUCAUC |
| | A-C | |
| | 38_6-6_internal_ | |
| | loop-symmetric_ | |
| | UGCAUC-AUUCUC | |
| 712 | -8_6-6_internal_ | GAUAUUGUGACAUAUCUCUGCACUCAU |
| | loop-symmetric_ | CACACAAAAAUAAACAAUCUUCUAUAG |
| | AGUUCA-CACGAG | GAUCCACAGGGAGGGGGCAUUUUAAU |
| | 38_6-6_internal_ | AUAUCUCCACGAGAAUCAUC |
| | loop-symmetric_ | |
| | UGCAUC-AUAAAC | |
| 713 | -8_6-6_internal_ | GAUAUUGUGACAUAUCUCUGCACUCAU |
| | loop-symmetric_ | CACACAAAUAUCAGAAAUCUUCUAUAG |
| | AGUUCA-GUCUAA | GAUCCACAGGGAGGGGGCAUUUUAAC |
| | 0_1-1_mismatch_ | AUAUCUCGUCUAAAAUCAUC |
| | A-C | |
| | 39_6-6_internal_ | |
| | loop-symmetric_ | |
| | GCAUCU-UAUCAG | |
| 714 | -8_6-6_internal_ | GAUAUUGUGACAUAUCUCUGCACUCAU |
| | loop-symmetric_ | CACACAAACCUGGAAAAUCUUCUAUAG |
| | AGUUCA-AUACCC | GAUCCACAGGGAGGGGGCAUUUCAAU |
| | 3_1-1_mismatch_ | AUAUCUCAUACCCAAUCAUC |
| | A-C | |
| | 39_6-6_internal_ | |
| | loop-symmetric_ | |
| | GCAUCU-CCUGGA | |
| 715 | -8_6-6_internal_ | GAUAUUGUGACAUAUCUCUGCACUCAU |
| | loop-symmetric_ | CACACAAACCUCGAAAAUCUUCUAUAG |
| | AGUUCA-CCGUAA | GAUCCACAGGGAGGGGGCAUUCUAAU |
| | 4_1-1_mismatch_ | AUAUCUCCCGUAAAAUCAUC |
| | A-C | |
| | 39_6-6_internal_ | |
| | loop-symmetric_ | |
| | GCAUCU-CCUCGA | |
| 716 | -8_6-6_internal_ | GAUAUUGUGACAUAUCUCUGCACUCAU |
| | loop-symmetric_ | CACACAAACCUACAAAAUCUUCUAUAG |
| | AGUUCA-ACACCC | GAUCCACAGGGAGGGGGCAUUUUAAU |
| | 39_6-6_internal_ | AUAUCUCACACCCAAUCAUC |
| | loop-symmetric_ | |
| | GCAUCU-CCUACA | |
| 717 | -8_6-6_internal_ | GAUAUUGUGACAUAUCUCUGCACUCAU |
| | loop-symmetric_ | CACACAAUCAGCCCAAAUCUUCUAUAG |
| | AGUUCA-ACCAGA | GAUCCACAGGGAGGGGGCAUUUUAAC |
| | 0_1-1_mismatch_ | AUAUCUCACCAGAAAUCAUC |
| | A-C | |
| | 40_6-6_internal_ | |
| | loop-symmetric_ | |
| | CAUCUU-UCAGCC | |
| 718 | -8_6-6_internal_ | GAUAUUGUGACAUAUCUCUGCACUCAU |
| | loop-symmetric_ | CACACAACGCAUUCAAAUCUUCUAUAG |
| | AGUUCA-CCGUAA | GAUCCACAGGGAGGGGGCAUUUCAAU |
| | 3_1-1_mismatch_ | AUAUCUCCCGUAAAAUCAUC |
| | A-C | |
| | 40_6-6_internal_ | |
| | loop-symmetric_ | |
| | CAUCUU-CGCAUU | |
| 719 | -8_6-6_internal_ | GAUAUUGUGACAUAUCUCUGCACUCAU |
| | loop-symmetric_ | CACACAACCCACACAAAUCUUCUAUAG |
| | AGUUCA-CAUUUC | GAUCCACAGGGAGGGGGCAUUCUAAU |
| | 4_1-1_mismatch_ | AUAUCUCCAUUUCAAUCAUC |
| | A-C | |
| | 40_6-6_internal_ | |
| | loop-symmetric_ | |
| | CAUCUU-CCCACA | |
| 720 | -8_6-6_internal_ | GAUAUUGUGACAUAUCUCUGCACUCAU |
| | loop-symmetric_ | CACACAACACACUCAAAUCUUCUAUAG |
| | AGUUCA-ACACCA | GAUCCACAGGGAGGGGGCAUCUUAAU |
| | 5_1-1_mismatch_ | AUAUCUCACACCAAAUCAUC |
| | A-C | |
| | 40_6-6_internal_ | |
| | loop-symmetric_ | |
| | CAUCUU-CACACU | |
| 721 | -8_6-6_internal_ | GAUAUUGUGACAUAUCUCUGCACUCAU |
| | loop-symmetric_ | CACACAACCCAUUCAAAUCUUCUAUAG |
| | AGUUCA-GACAAA | GAUCCACAGGGAGGGGGCAUUUUAAU |
| | 40_6-6_internal_ | AUAUCUCGACAAAAAUCAUC |
| | loop-symmetric_ | |
| | CAUCUU-CCCAUU | |
| 722 | -8_6-6_internal_ | GAUAUUGUGACAUAUCUCUGCACUCAU |
| | loop-symmetric_ | CACACAUCGCGCGCAAAUCUUCUAUAG |
| | AGUUCA-CUCCGA | GAUCCACAGGGAGGGGGCAUUUUAAC |
| | 0_1-1_mismatch_ | AUAUCUCCUCCGAAAUCAUC |
| | A-C | |
| | 41_6-6_internal_ | |
| | loop-symmetric_ | |
| | AUCUUU-UCGCGC | |
| 723 | -8_6-6_internal_ | GAUAUUGUGACAUAUCUCUGCACUCAU |
| | loop-symmetric_ | CACACAUGUCCAGCAAAUCUUCUAUAG |
| | AGUUCA-GUUCAA | GAUCCACAGGGAGGGGGCAUUUCAAU |
| | 3_1-1_mismatch_ | AUAUCUCGUUCAAAAUCAUC |
| | A-C | |
| | 41_6-6_internal_ | |
| | loop-symmetric_ | |
| | AUCUUU-UGUCCA | |
| 724 | -8_6-6_internal_ | GAUAUUGUGACAUAUCUCUGCACUCAU |
| | loop-symmetric_ | CACACACCUUGCGCAAAUCUUCUAUAG |
| | AGUUCA-GUUUUC | GAUCCACAGGGAGGGGGCAUUCUAAU |
| | 4_1-1_mismatch_ | AUAUCUCGUUUUCAAUCAUC |
| | A-C | |
| | 41_6-6_internal_ | |
| | loop-symmetric_ | |
| | AUCUUU-CCUUGC | |
| 725 | -8_6-6_internal_ | GAUAUUGUGACAUAUCUCUGCACUCAU |
| | loop-symmetric_ | CACACAUCGUCGGCAAAUCUUCUAUAG |
| | AGUUCA-CUUCCG | GAUCCACAGGGAGGGGGCAUCUUAAU |
| | 5_1-1_mismatch_ | AUAUCUCCUUCCGAAUCAUC |
| | A-C | |
| | 41_6-6_internal_ | |
| | loop-symmetric_ | |
| | AUCUUU-UCGUCG | |
| 726 | -8_6-6_internal_ | GAUAUUGUGACAUAUCUCUGCACUCAU |
| | loop-symmetric_ | CACACAUUACCGGCAAAUCUUCUAUAG |
| | AGUUCA-ACAGAG | GAUCCACAGGGAGGGGGCAUUUUAAU |
| | 41_6-6_internal_ | AUAUCUCACAGAGAAUCAUC |
| | loop-symmetric_ | |
| | AUCUUU-UUACCG | |
| 727 | -8_6-6_internal_ | GAUAUUGUGACAUAUCUCUGCACUCAU |
| | loop-symmetric_ | CACACCUCGAUUGCAAAUCUUCUAUAG |
| | AGUUCA-ACUUCG | GAUCCACAGGGAGGGGGCAUUUUAAC |
| | 0_1-1_mismatch_ | AUAUCUCACUUCGAAUCAUC |
| | A-C | |
| | 42_6-6_internal_ | |
| | loop-symmetric_ | |
| | UCUUUU-CUCGAU | |
| 728 | -8_6-6_internal_ | GAUAUUGUGACAUAUCUCUGCACUCAU |
| | loop-symmetric_ | CACACCUCCAUUGCAAAUCUUCUAUAG |
| | AGUUCA-CCACAA | GAUCCACAGGGAGGGGGCAUUUCAAU |
| | 3_1-1_mismatch_ | AUAUCUCCCACAAAAUCAUC |
| | A-C | |
| | 42_6-6_internal_ | |
| | loop-symmetric_ | |
| | UCUUUU-CUCCAU | |
| 729 | -8_6-6_internal_ | GAUAUUGUGACAUAUCUCUGCACUCAU |
| | loop-symmetric_ | CACACUGCGCUUGCAAAUCUUCUAUAG |
| | AGUUCA-ACACGG | GAUCCACAGGGAGGGGGCAUUCUAAU |
| | 4_1-1_mismatch_ | AUAUCUCACACGGAAUCAUC |
| | A-C | |
| | 42_6-6_internal_ | |
| | loop-symmetric_ | |
| | UCUUUU-UGCGCU | |
| 730 | -8_6-6_internal_ | GAUAUUGUGACAUAUCUCUGCACUCAU |
| | loop-symmetric_ | CACACUUCGUUUGCAAAUCUUCUAUAG |
| | AGUUCA-CAUUGG | GAUCCACAGGGAGGGGGCAUCUUAAU |
| | 5_1-1_mismatch_ | AUAUCUCCAUUGGAAUCAUC |
| | A-C | |
| | 42_6-6_internal_ | |
| | loop-symmetric_ | |
| | UCUUUU-UUCGUU | |
| 731 | -8_6-6_internal_ | GAUAUUGUGACAUAUCUCUGCACUCAU |
| | loop-symmetric_ | CACACUUAUCCUGCAAAUCUUCUAUAG |
| | AGUUCA-GGUGAG | GAUCCACAGGGAGGGGGCAUUUUAAU |
| | 42_6-6_internal_ | AUAUCUCGGUGAGAAUCAUC |
| | loop-symmetric_ | |
| | UCUUUU-UUAUCC | |
| 732 | -8_6-6_internal_ | GAUAUUGUGACAUAUCUCUGCACUCAU |
| | loop-symmetric_ | CACAACGCUAAUGCAAAUCUUCUAUAG |
| | AGUUCA-CACCAA | GAUCCACAGGGAGGGGGCAUUUUAAC |
| | 0_1-1_mismatch_ | AUAUCUCCACCAAAAUCAUC |
| | A-C | |
| | 43_6-6_internal_ | |
| | loop-symmetric_ | |
| | CUUUUG-ACGCUA | |
| 733 | -8_6-6_internal_ | GAUAUUGUGACAUAUCUCUGCACUCAU |
| | loop-symmetric_ | CACAGUCUGCAUGCAAAUCUUCUAUAG |
| | AGUUCA-CAUCGA | GAUCCACAGGGAGGGGGCAUUUCAAU |
| | 3_1-1_mismatch_ | AUAUCUCCAUCGAAAUCAUC |
| | A-C | |
| | 43_6-6_internal_ | |
| | loop-symmetric_ | |
| | CUUUUG-GUCUGC | |
| 734 | -8_6-6_internal_ | GAUAUUGUGACAUAUCUCUGCACUCAU |
| | loop-symmetric_ | CACAGUCCAAAUGCAAAUCUUCUAUAG |
| | AGUUCA-CUCCCC | GAUCCACAGGGAGGGGGCAUUCUAAU |
| | 4_1-1_mismatch_ | AUAUCUCCUCCCCAAUCAUC |
| | A-C | |
| | 43_6-6_internal_ | |
| | loop-symmetric_ | |
| | CUUUUG-GUCCAA | |
| 735 | -8_6-6_internal_ | GAUAUUGUGACAUAUCUCUGCACUCAU |
| | loop-symmetric_ | CACAAUUUGAAUGCAAAUCUUCUAUA |
| | AGUUCA-GACAGG | GGAUCCACAGGGAGGGGGCAUCUUAA |
| | 5_1-1_mismatch_ | UAUAUCUCGACAGGAAUCAUC |
| | A-C | |
| | 43_6-6_internal_ | |
| | loop-symmetric_ | |
| | CUUUUG-AUUUGA | |
| 736 | -8_6-6_internal_ | GAUAUUGUGACAUAUCUCUGCACUCAU |
| | loop-symmetric_ | CACAAUGUACAUGCAAAUCUUCUAUAG |
| | AGUUCA-GCGGGG | GAUCCACAGGGAGGGGGCAUUUUAAU |
| | 43_6-6_internal_ | AUAUCUCGCGGGGAAUCAUC |
| | loop-symmetric_ | |
| | CUUUUG-AUGUAC | |
| 737 | -8_6-6_internal_ | GAUAUUGUGACAUAUCUCUGCACUCAU |
| | loop-symmetric_ | CACUGCCGUGAUGCAAAUCUUCUAUAG |
| | AGUUCA-GGUUUC | GAUCCACAGGGAGGGGGCAUUUUAAC |
| | 0_1-1_mismatch_ | AUAUCUCGGUUUCAAUCAUC |
| | A-C | |
| | 44_6-6_internal_ | |
| | loop-symmetric_ | |
| | UUUUGU-UGCCGU | |
| 738 | -8_6-6_internal_ | GAUAUUGUGACAUAUCUCUGCACUCAU |
| | loop-symmetric_ | CACUUGGUUGAUGCAAAUCUUCUAUA |
| | AGUUCA-GUUUAA | GGAUCCACAGGGAGGGGGCAUUUCAA |
| | 3_1-1_mismatch_ | UAUAUCUCGUUUAAAAUCAUC |
| | A-C | |
| | 44_6-6_internal_ | |
| | loop-symmetric_ | |
| | UUUUGU-UUGGUU | |
| 739 | -8_6-6_internal_ | GAUAUUGUGACAUAUCUCUGCACUCAU |
| | loop-symmetric_ | CACCCCUUUGAUGCAAAUCUUCUAUAG |
| | AGUUCA-AAGUAG | GAUCCACAGGGAGGGGGCAUUCUAAU |
| | 4_1-1_mismatch_ | AUAUCUCAAGUAGAAUCAUC |
| | A-C | |
| | 44_6-6_internal_ | |
| | loop-symmetric_ | |
| | UUUUGU-CCCUUU | |
| 740 | -8_6-6_internal_ | GAUAUUGUGACAUAUCUCUGCACUCAU |
| | loop-symmetric_ | CACCUUUUCGAUGCAAAUCUUCUAUAG |
| | AGUUCA-GAUUCG | GAUCCACAGGGAGGGGGCAUCUUAAU |
| | 5_1-1_mismatch_ | AUAUCUCGAUUCGAAUCAUC |
| | A-C | |
| | 44_6-6_internal_ | |
| | loop-symmetric_ | |
| | UUUUGU-CUUUUC | |
| 741 | -8_6-6_internal_ | GAUAUUGUGACAUAUCUCUGCACUCAU |
| | loop-symmetric_ | CACUGUGCUGAUGCAAAUCUUCUAUAG |
| | AGUUCA-CCAUGG | GAUCCACAGGGAGGGGGCAUUUUAAU |
| | 44_6-6_internal_ | AUAUCUCCCAUGGAAUCAUC |
| | loop-symmetric_ | |
| | UUUUGU-UGUGCU | |
| 742 | -7_6-6_internal_ | GAUAUUGUGACAUAUCUCUGCACUCAU |
| | loop-symmetric_ | CACACAAAAGAUGCAAAUCUUCUAUAG |
| | GUUCAG-GGCUCA | GAUCCACAGGGAGGGGGCAUUUUAAC |
| | 0_1-1_mismatch_ | AUAUCUGGCUCAUAAUCAUC |
| | A-C | |
| 743 | -7_6-6_internal_ | GAUAUUGUGACAUAUCUCUGCACUCAU |
| | loop-symmetric_ | CACACAAAAGAUGCAAAUCUUCUAUAG |
| | GUUCAG-GCCGCA | GAUCCACAGGGAGGGGGCAUUUCAAU |
| | 3_1-1_mismatch_ | AUAUCUGCCGCAUAAUCAUC |
| | A-C | |
| 744 | -7_6-6_internal_ | GAUAUUGUGACAUAUCUCUGCACUCAU |
| | loop-symmetric_ | CACACAAAAGAUGCAAAUCUUCUAUAG |
| | GUUCAG-AACUAA | GAUCCACAGGGAGGGGGCAUUCUAAU |
| | 4_1-1_mismatch_ | AUAUCUAACUAAUAAUCAUC |
| | A-C | |
| 745 | -7_6-6_internal_ | GAUAUUGUGACAUAUCUCUGCACUCAU |
| | loop-symmetric_ | CACACAAAAGAUGCAAAUCUUCUAUAG |
| | GUUCAG-AACACA | GAUCCACAGGGAGGGGGCAUCUUAAU |
| | 5_1-1_mismatch_ | AUAUCUAACACAUAAUCAUC |
| | A-C | |
| 746 | -7_6-6_internal_ | GAUAUUGUGACAUAUCUCUGCACUCAU |
| | loop-symmetric_ | CACACAAAAGAUGCAAAUCUUCUAUAG |
| | GUUCAG-GGCCCG | GAUCCACAGGGAGGGGGCAUUUUAAU |
| | | AUAUCUGGCCCGUAAUCAUC |
| 747 | -7_6-6_internal_ | GAUAUUGUGACAUAUCUCUGCACUCAU |
| | loop-symmetric_ | CACACAAAAGAUGCAAAUCUUCUAUAG |
| | GUUCAG-AAAUUG | AAAAGUCAGGGAGGGGGCAUUUCAAU |
| | 3_1-1_mismatch_ | AUAUCUAAAUUGUAAUCAUC |
| | A-C | |
| | 20_6-6_internal_ | |
| | loop-symmetric_ | |
| | UGGAUC-AAAAGU | |
| 748 | -7_6-6_internal_ | GAUAUUGUGACAUAUCUCUGCACUCAU |
| | loop-symmetric_ | CACACAAAAGAUGCAAAUCUUCUAUAG |
| | GUUCAG-ACGUCG | CGCAAUCAGGGAGGGGGCAUCUUAAU |
| | 5_1-1_mismatch_ | AUAUCUACGUCGUAAUCAUC |
| | A-C | |
| | 20_6-6_internal_ | |
| | loop-symmetric_ | |
| | UGGAUC-CGCAAU | |
| 749 | -7_6-6_internal_ | GAUAUUGUGACAUAUCUCUGCACUCAU |
| | loop-symmetric_ | CACACAAAAGAUGCAAAUCUUCUAUAG |
| | GUUCAG-GGUCAA | CUCAGUCAGGGAGGGGGCAUUUUAAU |
| | 20_6-6_internal_ | AUAUCUGGUCAAUAAUCAUC |
| | loop-symmetric_ | |
| | UGGAUC-CUCAGU | |
| 750 | -7_6-6_internal_ | GAUAUUGUGACAUAUCUCUGCACUCAU |
| | loop-symmetric_ | CACACAAAAGAUGCAAAUCUUCUAUAA |
| | GUUCAG-AAAUCG | CUGAGACAGGGAGGGGGCAUUUUAAC |
| | 0_1-1_mismatch_ | AUAUCUAAAUCGUAAUCAUC |
| | A-C | |
| | 21_6-6_internal_ | |
| | loop-symmetric_ | |
| | GGAUCC-ACUGAG | |
| 751 | -7_6-6_internal_ | GAUAUUGUGACAUAUCUCUGCACUCAU |
| | loop-symmetric_ | CACACAAAAGAUGCAAAUCUUCUAUAU |
| | GUUCAG-ACAAUA | CUCAAACAGGGAGGGGGCAUUUCAAU |
| | 3_1-1_mismatch_ | AUAUCUACAAUAUAAUCAUC |
| | A-C | |
| | 21_6-6_internal_ | |
| | loop-symmetric_ | |
| | GGAUCC-UCUCAA | |
| 752 | -7_6-6_internal_ | GAUAUUGUGACAUAUCUCUGCACUCAU |
| | loop-symmetric_ | CACACAAAAGAUGCAAAUCUUCUAUAU |
| | GUUCAG-ACGCUA | UAAGAACAGGGAGGGGGCAUCUUAAU |
| | 5_1-1_mismatch_ | AUAUCUACGCUAUAAUCAUC |
| | A-C | |
| | 21_6-6_internal_ | |
| | loop-symmetric_ | |
| | GGAUCC-UUAAGA | |
| 753 | -7_6-6_internal_ | GAUAUUGUGACAUAUCUCUGCACUCAU |
| | loop-symmetric_ | CACACAAAAGAUGCAAAUCUUCUAUAA |
| | GUUCAG-AUUCCA | CCUAAACAGGGAGGGGGCAUUUUAAU |
| | 21_6-6_internal_ | AUAUCUAUUCCAUAAUCAUC |
| | loop-symmetric_ | |
| | GGAUCC-ACCUAA | |
| 754 | -7_6-6_internal_ | GAUAUUGUGACAUAUCUCUGCACUCAU |
| | loop-symmetric_ | CACACAAAAGAUGCAAAUCUUCUAUUA |
| | GUUCAG-AACCUG | UUGACACAGGGAGGGGGCAUUUUAAC |
| | 0_1-1_mismatch_ | AUAUCUAACCUGUAAUCAUC |
| | A-C | |
| | 22_6-6_internal_ | |
| | loop-symmetric_ | |
| | GAUCCU-UAUUGA | |
| 755 | -7_6-6_internal_ | GAUAUUGUGACAUAUCUCUGCACUCAU |
| | loop-symmetric_ | CACACAAAAGAUGCAAAUCUUCUAUUA |
| | GUUCAG-GGUCGA | CUAGCACAGGGAGGGGGCAUUUCAAU |
| | 3_1-1_mismatch_ | AUAUCUGGUCGAUAAUCAUC |
| | A-C | |
| | 22_6-6_internal_ | |
| | loop-symmetric_ | |
| | GAUCCU-UACUAG | |
| 756 | -7_6-6_internal_ | GAUAUUGUGACAUAUCUCUGCACUCAU |
| | loop-symmetric_ | CACACAAAAGAUGCAAAUCUUCUAUCU |
| | GUUCAG-GCUUGA | AGCACACAGGGAGGGGGCAUUCUAAU |
| | 4_1-1_mismatch_ | AUAUCUGCUUGAUAAUCAUC |
| | A-C | |
| | 22_6-6_internal_ | |
| | loop-symmetric_ | |
| | GAUCCU-CUAGCA | |
| 757 | -7_6-6_internal_ | GAUAUUGUGACAUAUCUCUGCACUCAU |
| | loop-symmetric_ | CACACAAAAGAUGCAAAUCUUCUAUUU |
| | GUUCAG-AAGCAA | CCCGCACAGGGAGGGGGCAUCUUAAUA |
| | 5_1-1_mismatch_ | UAUCUAAGCAAUAAUCAUC |
| | A-C | |
| | 22_6-6_internal_ | |
| | loop-symmetric_ | |
| | GAUCCU-UUCCCG | |
| 758 | -7_6-6_internal_ | GAUAUUGUGACAUAUCUCUGCACUCAU |
| | loop-symmetric_ | CACACAAAAGAUGCAAAUCUUCUAUCA |
| | GUUCAG-AAUUCG | CCGGCACAGGGAGGGGGCAUUUUAAU |
| | 22_6-6_internal_ | AUAUCUAAUUCGUAAUCAUC |
| | loop-symmetric_ | |
| | GAUCCU-CACCGG | |
| 759 | -7_6-6_internal_ | GAUAUUGUGACAUAUCUCUGCACUCAU |
| | loop-symmetric_ | CACACAAAAGAUGCAAAUCUUCUAGCC |
| | GUUCAG-GGCUCA | UACCCACAGGGAGGGGGCAUUUUAACA |
| | 0_1-1_mismatch_ | UAUCUGGCUCAUAAUCAUC |
| | A-C | |
| | 23_6-6_internal_ | |
| | loop-symmetric_ | |
| | AUCCUA-GCCUAC | |
| 760 | -7_6-6_internal_ | GAUAUUGUGACAUAUCUCUGCACUCAU |
| | loop-symmetric_ | CACACAAAAGAUGCAAAUCUUCUAGCU |
| | GUUCAG-GACCCA | CGCCCACAGGGAGGGGGCAUUUCAAUA |
| | 3_1-1_mismatch_ | UAUCUGACCCAUAAUCAUC |
| | A-C | |
| | 23_6-6_internal_ | |
| | loop-symmetric_ | |
| | AUCCUA-GCUCGC | |
| 761 | -7_6-6_internal_ | GAUAUUGUGACAUAUCUCUGCACUCAU |
| | loop-symmetric_ | CACACAAAAGAUGCAAAUCUUCUAACA |
| | GUUCAG-GGCUUA | ACGCCACAGGGAGGGGGCAUUCUAAUA |
| | 4_1-1_mismatch_ | UAUCUGGCUUAUAAUCAUC |
| | A-C | |
| | 23_6-6_internal_ | |
| | loop-symmetric_ | |
| | AUCCUA-ACAACG | |
| 762 | -7_6-6_internal_ | GAUAUUGUGACAUAUCUCUGCACUCAU |
| | loop-symmetric_ | CACACAAAAGAUGCAAAUCUUCUAAUC |
| | GUUCAG-GAAGUG | AGCCCACAGGGAGGGGGCAUCUUAAUA |
| | 5_1-1_mismatch_ | UAUCUGAAGUGUAAUCAUC |
| | A-C | |
| | 23_6-6_internal_ | |
| | loop-symmetric_ | |
| | AUCCUA-AUCAGC | |
| 763 | -7_6-6_internal_ | GAUAUUGUGACAUAUCUCUGCACUCAU |
| | loop-symmetric_ | CACACAAAAGAUGCAAAUCUUCUAGCC |
| | GUUCAG-AUCACG | ACCCCACAGGGAGGGGGCAUUUUAAUA |
| | 23_6-6_internal_ | UAUCUAUCACGUAAUCAUC |
| | loop-symmetric_ | |
| | AUCCUA-GCCACC | |
| 764 | -7_6-6_internal_ | GAUAUUGUGACAUAUCUCUGCACUCAU |
| | loop-symmetric_ | CACACAAAAGAUGCAAAUCUUCUCUCG |
| | GUUCAG-GCUCCG | UCUCCACAGGGAGGGGGCAUUUUAACA |
| | 0_1-1_mismatch_ | UAUCUGCUCCGUAAUCAUC |
| | A-C | |
| | 24_6-6_internal_ | |
| | loop-symmetric_ | |
| | UCCUAU-CUCGUC | |
| 765 | -7_6-6_internal_ | GAUAUUGUGACAUAUCUCUGCACUCAU |
| | loop-symmetric_ | CACACAAAAGAUGCAAAUCUUCUCCAU |
| | GUUCAG-GGUCCA | CUUCCACAGGGAGGGGGCAUUUCAAUA |
| | 3_1-1_mismatch_ | UAUCUGGUCCAUAAUCAUC |
| | A-C | |
| | 24_6-6_internal_ | |
| | loop-symmetric_ | |
| | UCCUAU-CCAUCU | |
| 766 | -7_6-6_internal_ | GAUAUUGUGACAUAUCUCUGCACUCAU |
| | loop-symmetric_ | CACACAAAAGAUGCAAAUCUUCUCACA |
| | GUUCAG-GGCGUG | AUUCCACAGGGAGGGGGCAUUCUAAU |
| | 4_1-1_mismatch_ | AUAUCUGGCGUGUAAUCAUC |
| | A-C | |
| | 24_6-6_internal_ | |
| | loop-symmetric_ | |
| | UCCUAU-CACAAU | |
| 767 | -7_6-6_internal_ | GAUAUUGUGACAUAUCUCUGCACUCAU |
| | loop-symmetric_ | CACACAAAAGAUGCAAAUCUUCUUAAC |
| | GUUCAG-ACUUGA | GUUCCACAGGGAGGGGGCAUCUUAAU |
| | 5_1-1_mismatch_ | AUAUCUACUUGAUAAUCAUC |
| | A-C | |
| | 24_6-6_internal_ | |
| | loop-symmetric_ | |
| | UCCUAU-UAACGU | |
| 768 | -7_6-6_internal_ | GAUAUUGUGACAUAUCUCUGCACUCAU |
| | loop-symmetric_ | CACACAAAAGAUGCAAAUCUUCUUGUA |
| | GUUCAG-AAGCCA | UUUCCACAGGGAGGGGGCAUUUUAAU |
| | 24_6-6_internal_ | AUAUCUAAGCCAUAAUCAUC |
| | loop-symmetric_ | |
| | UCCUAU-UGUAUU | |
| 769 | -7_6-6_internal_ | GAUAUUGUGACAUAUCUCUGCACUCAU |
| | loop-symmetric_ | CACACAAAAGAUGCAAAUCUUCGACUC |
| | GUUCAG-AAAAUA | CAUCCACAGGGAGGGGGCAUUUUAACA |
| | 0_1-1_mismatch_ | UAUCUAAAAUAUAAUCAUC |
| | A-C | |
| | 25_6-6_internal_ | |
| | loop-symmetric_ | |
| | CCUAUA-GACUCC | |
| 770 | -7_6-6_internal_ | GAUAUUGUGACAUAUCUCUGCACUCAU |
| | loop-symmetric_ | CACACAAAAGAUGCAAAUCUUCGAAGA |
| | GUUCAG-GCCGCA | AAUCCACAGGGAGGGGGCAUUUCAAU |
| | 3_1-1_mismatch_ | AUAUCUGCCGCAUAAUCAUC |
| | A-C | |
| | 25_6-6_internal_ | |
| | loop-symmetric_ | |
| | CCUAUA-GAAGAA | |
| 771 | -7_6-6_internal_ | GAUAUUGUGACAUAUCUCUGCACUCAU |
| | loop-symmetric_ | CACACAAAAGAUGCAAAUCUUCAUCAC |
| | GUUCAG-AAAUUA | CAUCCACAGGGAGGGGGCAUUCUAAUA |
| | 4_1-1_mismatch_ | UAUCUAAAUUAUAAUCAUC |
| | A-C | |
| | 25_6-6_internal_ | |
| | loop-symmetric_ | |
| | CCUAUA-AUCACC | |
| 772 | -7_6-6_internal_ | GAUAUUGUGACAUAUCUCUGCACUCAU |
| | loop-symmetric_ | CACACAAAAGAUGCAAAUCUUCACGUU |
| | GUUCAG-ACUAUG | UAUCCACAGGGAGGGGGCAUCUUAAU |
| | 5_1-1_mismatch_ | AUAUCUACUAUGUAAUCAUC |
| | A-C | |
| | 25_6-6_internal_ | |
| | loop-symmetric_ | |
| | CCUAUA-ACGUUU | |
| 773 | -7_6-6_internal_ | GAUAUUGUGACAUAUCUCUGCACUCAU |
| | loop-symmetric_ | CACACAAAAGAUGCAAAUCUUCCCCUU |
| | GUUCAG-AUUUUG | UAUCCACAGGGAGGGGGCAUUUUAAU |
| | 25_6-6_internal_ | AUAUCUAUUUUGUAAUCAUC |
| | loop-symmetric_ | |
| | CCUAUA-CCCUUU | |
| 774 | -7_6-6_internal_ | GAUAUUGUGACAUAUCUCUGCACUCAU |
| | loop-symmetric_ | CACACAAAAGAUGCAAAUCUUGGAAU |
| | GUUCAG-ACUUGA | AGAUCCACAGGGAGGGGGCAUUUUAA |
| | 0_1-1_mismatch_ | CAUAUCUACUUGAUAAUCAUC |
| | A-C | |
| | 26_6-6_internal_ | |
| | loop-symmetric_ | |
| | CUAUAG-GGAAUA | |
| 775 | -7_6-6_internal_ | GAUAUUGUGACAUAUCUCUGCACUCAU |
| | loop-symmetric_ | CACACAAAAGAUGCAAAUCUUGAACGC |
| | GUUCAG-GCACUG | GAUCCACAGGGAGGGGGCAUUUCAAU |
| | 3_1-1_mismatch_ | AUAUCUGCACUGUAAUCAUC |
| | A-C | |
| | 26_6-6_internal_ | |
| | loop-symmetric_ | |
| | CUAUAG-GAACGC | |
| 776 | -7_6-6_internal_ | GAUAUUGUGACAUAUCUCUGCACUCAU |
| | loop-symmetric_ | CACACAAAAGAUGCAAAUCUUGAUAUC |
| | GUUCAG-AGUUAA | GAUCCACAGGGAGGGGGCAUUCUAAU |
| | 4_1-1_mismatch_ | AUAUCUAGUUAAUAAUCAUC |
| | A-C | |
| | 26_6-6_internal_ | |
| | loop-symmetric_ | |
| | CUAUAG-GAUAUC | |
| 777 | -7_6-6_internal_ | GAUAUUGUGACAUAUCUCUGCACUCAU |
| | loop-symmetric_ | CACACAAAAGAUGCAAAUCUUAUACCU |
| | GUUCAG-AUAGUG | GAUCCACAGGGAGGGGGCAUCUUAAU |
| | 5_1-1_mismatch_ | AUAUCUAUAGUGUAAUCAUC |
| | A-C | |
| | 26_6-6_internal_ | |
| | loop-symmetric_ | |
| | CUAUAG-AUACCU | |
| 778 | -7_6-6_internal_ | GAUAUUGUGACAUAUCUCUGCACUCAU |
| | loop-symmetric_ | CACACAAAAGAUGCAAAUCUUAGGCUC |
| | GUUCAG-AACACA | GAUCCACAGGGAGGGGGCAUUUUAAU |
| | 26_6-6_internal_ | AUAUCUAACACAUAAUCAUC |
| | loop-symmetric_ | |
| | CUAUAG-AGGCUC | |
| 779 | -7_6-6_internal_ | GAUAUUGUGACAUAUCUCUGCACUCAU |
| | loop-symmetric_ | CACACAAAAGAUGCAAAUCUAGGCGUG |
| | GUUCAG-AACUCG | GAUCCACAGGGAGGGGGCAUUUUAAC |
| | 0_1-1_mismatch_ | AUAUCUAACUCGUAAUCAUC |
| | A-C | |
| | 27_6-6_internal_ | |
| | loop-symmetric_ | |
| | UAUAGA-AGGCGU | |
| 780 | -7_6-6_internal_ | GAUAUUGUGACAUAUCUCUGCACUCAU |
| | loop-symmetric_ | CACACAAAAGAUGCAAAUCUGUGUUU |
| | GUUCAG-AGUUAA | GGAUCCACAGGGAGGGGGCAUUUCAA |
| | 3_1-1_mismatch_ | UAUAUCUAGUUAAUAAUCAUC |
| | A-C | |
| | 27_6-6_internal_ | |
| | loop-symmetric_ | |
| | UAUAGA-GUGUUU | |
| 781 | -7_6-6_internal_ | GAUAUUGUGACAUAUCUCUGCACUCAU |
| | loop-symmetric_ | CACACAAAAGAUGCAAAUCUCAUCAUG |
| | GUUCAG-ACCUCA | GAUCCACAGGGAGGGGGCAUUCUAAU |
| | 4_1-1_mismatch_ | AUAUCUACCUCAUAAUCAUC |
| | A-C | |
| | 27_6-6_internal_ | |
| | loop-symmetric_ | |
| | UAUAGA-CAUCAU | |
| 782 | -7_6-6_internal_ | GAUAUUGUGACAUAUCUCUGCACUCAU |
| | loop-symmetric_ | CACACAAAAGAUGCAAAUCUAGCAGUG |
| | GUUCAG-GGCCUA | GAUCCACAGGGAGGGGGCAUCUUAAU |
| | 5_1-1_mismatch_ | AUAUCUGGCCUAUAAUCAUC |
| | A-C | |
| | 27_6-6_internal_ | |
| | loop-symmetric_ | |
| | UAUAGA-AGCAGU | |
| 783 | -7_6-6_internal_ | GAUAUUGUGACAUAUCUCUGCACUCAU |
| | loop-symmetric_ | CACACAAAAGAUGCAAAUCUCCACUCG |
| | GUUCAG-GACCUA | GAUCCACAGGGAGGGGGCAUUUUAAU |
| | 27_6-6_internal_ | AUAUCUGACCUAUAAUCAUC |
| | loop-symmetric_ | |
| | UAUAGA-CCACUC | |
| 784 | -7_6-6_internal_ | GAUAUUGUGACAUAUCUCUGCACUCAU |
| | loop-symmetric_ | CACACAAAAGAUGCAAAUCCAGUUCAG |
| | GUUCAG-ACCUUG | GAUCCACAGGGAGGGGGCAUUUUAAC |
| | 0_1-1_mismatch_ | AUAUCUACCUUGUAAUCAUC |
| | A-C | |
| | 28_6-6_internal_ | |
| | loop-symmetric_ | |
| | AUAGAA-CAGUUC | |
| 785 | -7_6-6_internal_ | GAUAUUGUGACAUAUCUCUGCACUCAU |
| | loop-symmetric_ | CACACAAAAGAUGCAAAUCGGAUCGAG |
| | GUUCAG-AGUUUG | GAUCCACAGGGAGGGGGCAUUUCAAU |
| | 3_1-1_mismatch_ | AUAUCUAGUUUGUAAUCAUC |
| | A-C | |
| | 28_6-6_internal_ | |
| | loop-symmetric_ | |
| | AUAGAA-GGAUCG | |
| 786 | -7_6-6_internal_ | GAUAUUGUGACAUAUCUCUGCACUCAU |
| | loop-symmetric_ | CACACAAAAGAUGCAAAUCGUGAUGA |
| | GUUCAG-GGCGCG | GGAUCCACAGGGAGGGGGCAUUCUAA |
| | 4_1-1_mismatch_ | UAUAUCUGGCGCGUAAUCAUC |
| | A-C | |
| | 28_6-6_internal_ | |
| | loop-symmetric_ | |
| | AUAGAA-GUGAUG | |
| 787 | -7_6-6_internal_ | GAUAUUGUGACAUAUCUCUGCACUCAU |
| | loop-symmetric_ | CACACAAAAGAUGCAAAUCGACCUCAG |
| | GUUCAG-GCGCGA | GAUCCACAGGGAGGGGGCAUCUUAAU |
| | 5_1-1_mismatch_ | AUAUCUGCGCGAUAAUCAUC |
| | A-C | |
| | 28_6-6_internal_ | |
| | loop-symmetric_ | |
| | AUAGAA-GACCUC | |
| 788 | -7_6-6_internal_ | GAUAUUGUGACAUAUCUCUGCACUCAU |
| | loop-symmetric_ | CACACAAAAGAUGCAAAUCCGAGCAAG |
| | GUUCAG-GCAGUG | GAUCCACAGGGAGGGGGCAUUUUAAU |
| | 28_6-6_internal_ | AUAUCUGCAGUGUAAUCAUC |
| | loop-symmetric_ | |
| | AUAGAA-CGAGCA | |
| 789 | -7_6-6_internal_ | GAUAUUGUGACAUAUCUCUGCACUCAU |
| | loop-symmetric_ | CACACAAAAGAUGCAAAUAUGAUCUA |
| | GUUCAG-AGUAUA | GGAUCCACAGGGAGGGGGCAUUUUAA |
| | 0_1-1_mismatch_ | CAUAUCUAGUAUAUAAUCAUC |
| | A-C | |
| | 29_6-6_internal_ | |
| | loop-symmetric_ | |
| | UAGAAG-AUGAUC | |
| 790 | -7_6-6_internal_ | GAUAUUGUGACAUAUCUCUGCACUCAU |
| | loop-symmetric_ | CACACAAAAGAUGCAAAUGGUGCCUAG |
| | GUUCAG-GAUGUA | GAUCCACAGGGAGGGGGCAUUUCAAU |
| | 3_1-1_mismatch_ | AUAUCUGAUGUAUAAUCAUC |
| | A-C | |
| | 29_6-6_internal_ | |
| | loop-symmetric_ | |
| | UAGAAG-GGUGCC | |
| 791 | -7_6-6_internal_ | GAUAUUGUGACAUAUCUCUGCACUCAU |
| | loop-symmetric_ | CACACAAAAGAUGCAAAUACAGGUUA |
| | GUUCAG-AACGGA | GGAUCCACAGGGAGGGGGCAUUCUAA |
| | 4_1-1_mismatch_ | UAUAUCUAACGGAUAAUCAUC |
| | A-C | |
| | 29_6-6_internal_ | |
| | loop-symmetric_ | |
| | UAGAAG-ACAGGU | |
| 792 | -7_6-6_internal_ | GAUAUUGUGACAUAUCUCUGCACUCAU |
| | loop-symmetric_ | CACACAAAAGAUGCAAAUGACGUUUA |
| | GUUCAG-ACGUUG | GGAUCCACAGGGAGGGGGCAUCUUAA |
| | 5_1-1_mismatch_ | UAUAUCUACGUUGUAAUCAUC |
| | A-C | |
| | 29_6-6_internal_ | |
| | loop-symmetric_ | |
| | UAGAAG-GACGUU | |
| 793 | -7_6-6_internal_ | GAUAUUGUGACAUAUCUCUGCACUCAU |
| | loop-symmetric_ | CACACAAAAGAUGCAAAUAUCAAUUA |
| | GUUCAG-GACCUA | GGAUCCACAGGGAGGGGGCAUUUUAA |
| | 29_6-6_internal_ | UAUAUCUGACCUAUAAUCAUC |
| | loop-symmetric_ | |
| | UAGAAG-AUCAAU | |
| 794 | -7_6-6_internal_ | GAUAUUGUGACAUAUCUCUGCACUCAU |
| | loop-symmetric_ | CACACAAAAGAUGCAAAAGAACGAUA |
| | GUUCAG-ACCAUG | GGAUCCACAGGGAGGGGGCAUUUUAA |
| | 0_1-1_mismatch_ | CAUAUCUACCAUGUAAUCAUC |
| | A-C | |
| | 30_6-6_internal_ | |
| | loop-symmetric_ | |
| | AGAAGA-AGAACG | |
| 795 | -7_6-6_internal_ | GAUAUUGUGACAUAUCUCUGCACUCAU |
| | loop-symmetric_ | CACACAAAAGAUGCAAAGGGUGGAUA |
| | GUUCAG-AGCUCA | GGAUCCACAGGGAGGGGGCAUUUCAA |
| | 3_1-1_mismatch_ | UAUAUCUAGCUCAUAAUCAUC |
| | A-C | |
| | 30_6-6_internal_ | |
| | loop-symmetric_ | |
| | AGAAGA-GGGUGG | |
| 796 | -7_6-6_internal_ | GAUAUUGUGACAUAUCUCUGCACUCAU |
| | loop-symmetric_ | CACACAAAAGAUGCAAAGAGCCGAUAG |
| | GUUCAG-GGCCUA | GAUCCACAGGGAGGGGGCAUUCUAAU |
| | 4_1-1_mismatch_ | AUAUCUGGCCUAUAAUCAUC |
| | A-C | |
| | 30_6-6_internal_ | |
| | loop-symmetric_ | |
| | AGAAGA-GAGCCG | |
| 797 | -7_6-6_internal_ | GAUAUUGUGACAUAUCUCUGCACUCAU |
| | loop-symmetric_ | CACACAAAAGAUGCAAAAUGGGGAUA |
| | GUUCAG-GGCUUG | GGAUCCACAGGGAGGGGGCAUCUUAA |
| | 5_1-1_mismatch_ | UAUAUCUGGCUUGUAAUCAUC |
| | A-C | |
| | 30_6-6_internal_ | |
| | loop-symmetric_ | |
| | AGAAGA-AUGGGG | |
| 798 | -7_6-6_internal_ | GAUAUUGUGACAUAUCUCUGCACUCAU |
| | loop-symmetric_ | CACACAAAAGAUGCAAAAUACACAUAG |
| | GUUCAG-GACUCA | GAUCCACAGGGAGGGGGCAUUUUAAU |
| | 30_6-6_internal_ | AUAUCUGACUCAUAAUCAUC |
| | loop-symmetric_ | |
| | AGAAGA-AUACAC | |
| 799 | -7_6-6_internal_ | GAUAUUGUGACAUAUCUCUGCACUCAU |
| | loop-symmetric_ | CACACAAAAGAUGCAACGGAAAUAUA |
| | GUUCAG-AAUUUA | GGAUCCACAGGGAGGGGGCAUUUUAA |
| | 0_1-1_mismatch_ | CAUAUCUAAUUUAUAAUCAUC |
| | A-C | |
| | 31_6-6_internal_ | |
| | loop-symmetric_ | |
| | GAAGAU-CGGAAA | |
| 800 | -7_6-6_internal_ | GAUAUUGUGACAUAUCUCUGCACUCAU |
| | loop-symmetric_ | CACACAAAAGAUGCAACAGGUAUAUA |
| | GUUCAG-GGCGGA | GGAUCCACAGGGAGGGGGCAUUUCAA |
| | 3_1-1_mismatch_ | UAUAUCUGGCGGAUAAUCAUC |
| | A-C | |
| | 31_6-6_internal_ | |
| | loop-symmetric_ | |
| | GAAGAU-CAGGUA | |
| 801 | -7_6-6_internal_ | GAUAUUGUGACAUAUCUCUGCACUCAU |
| | loop-symmetric_ | CACACAAAAGAUGCAACCGACGUAUAG |
| | GUUCAG-AACUCG | GAUCCACAGGGAGGGGGCAUUCUAAU |
| | 4_1-1_mismatch_ | AUAUCUAACUCGUAAUCAUC |
| | A-C | |
| | 31_6-6_internal_ | |
| | loop-symmetric_ | |
| | GAAGAU-CCGACG | |
| 802 | -7_6-6_internal_ | GAUAUUGUGACAUAUCUCUGCACUCAU |
| | loop-symmetric_ | CACACAAAAGAUGCAAUUGCCAUAUAG |
| | GUUCAG-AUUUUG | GAUCCACAGGGAGGGGGCAUCUUAAU |
| | 5_1-1_mismatch_ | AUAUCUAUUUUGUAAUCAUC |
| | A-C | |
| | 31_6-6_internal_ | |
| | loop-symmetric_ | |
| | GAAGAU-UUGCCA | |
| 803 | -7_6-6_internal_ | GAUAUUGUGACAUAUCUCUGCACUCAU |
| | loop-symmetric_ | CACACAAAAGAUGCAACGUACAUAUAG |
| | GUUCAG-GAUUGA | GAUCCACAGGGAGGGGGCAUUUUAAU |
| | 31_6-6_internal_ | AUAUCUGAUUGAUAAUCAUC |
| | loop-symmetric_ | |
| | GAAGAU-CGUACA | |
| 804 | -7_6-6_internal_ | GAUAUUGUGACAUAUCUCUGCACUCAU |
| | loop-symmetric_ | CACACAAAAGAUGCAUAAGCACUAUAG |
| | GUUCAG-GAAAUA | GAUCCACAGGGAGGGGGCAUUUUAAC |
| | 0_1-1_mismatch_ | AUAUCUGAAAUAUAAUCAUC |
| | A-C | |
| | 32_6-6_internal_ | |
| | loop-symmetric_ | |
| | AAGAUU-UAAGCA | |
| 805 | -7_6-6_internal_ | GAUAUUGUGACAUAUCUCUGCACUCAU |
| | loop-symmetric_ | CACACAAAAGAUGCACUAAGCCUAUAG |
| | GUUCAG-GCAGUG | GAUCCACAGGGAGGGGGCAUUUCAAU |
| | 3_1-1_mismatch_ | AUAUCUGCAGUGUAAUCAUC |
| | A-C | |
| | 32_6-6_internal_ | |
| | loop-symmetric_ | |
| | AAGAUU-CUAAGC | |
| 806 | -7_6-6_internal_ | GAUAUUGUGACAUAUCUCUGCACUCAU |
| | loop-symmetric_ | CACACAAAAGAUGCACCUGCCCUAUAG |
| | GUUCAG-GGCUAA | GAUCCACAGGGAGGGGGCAUUCUAAU |
| | 4_1-1_mismatch_ | AUAUCUGGCUAAUAAUCAUC |
| | A-C | |
| | 32_6-6_internal_ | |
| | loop-symmetric_ | |
| | AAGAUU-CCUGCC | |
| 807 | -7_6-6_internal_ | GAUAUUGUGACAUAUCUCUGCACUCAU |
| | loop-symmetric_ | CACACAAAAGAUGCAUACGCGCUAUAG |
| | GUUCAG-ACCCCG | GAUCCACAGGGAGGGGGCAUCUUAAU |
| | 5_1-1_mismatch_ | AUAUCUACCCCGUAAUCAUC |
| | A-C | |
| | 32_6-6_internal_ | |
| | loop-symmetric_ | |
| | AAGAUU-UACGCG | |
| 808 | -7_6-6_internal_ | GAUAUUGUGACAUAUCUCUGCACUCAU |
| | loop-symmetric_ | CACACAAAAGAUGCAUCCACACUAUAG |
| | GUUCAG-AACGCA | GAUCCACAGGGAGGGGGCAUUUUAAU |
| | 32_6-6_internal_ | AUAUCUAACGCAUAAUCAUC |
| | loop-symmetric_ | |
| | AAGAUU-UCCACA | |
| 809 | -7_6-6_internal_ | GAUAUUGUGACAUAUCUCUGCACUCAU |
| | loop-symmetric_ | CACACAAAAGAUGCUAUAAAUCUAUA |
| | GUUCAG-GCCCGA | GGAUCCACAGGGAGGGGGCAUUUUAA |
| | 0_1-1_mismatch_ | CAUAUCUGCCCGAUAAUCAUC |
| | A-C | |
| | 33_6-6_internal_ | |
| | loop-symmetric_ | |
| | AGAUUU-UAUAAA | |
| 810 | -7_6-6_internal_ | GAUAUUGUGACAUAUCUCUGCACUCAU |
| | loop-symmetric_ | CACACAAAAGAUGCCCUAUAUCUAUAG |
| | GUUCAG-AGUUUA | GAUCCACAGGGAGGGGGCAUUUCAAU |
| | 3_1-1_mismatch_ | AUAUCUAGUUUAUAAUCAUC |
| | A-C | |
| | 33_6-6_internal_ | |
| | loop-symmetric_ | |
| | AGAUUU-CCUAUA | |
| 811 | -7_6-6_internal_ | GAUAUUGUGACAUAUCUCUGCACUCAU |
| | loop-symmetric_ | CACACAAAAGAUGCCCCGACUCUAUAG |
| | GUUCAG-AGCCUA | GAUCCACAGGGAGGGGGCAUUCUAAU |
| | 4_1-1_mismatch_ | AUAUCUAGCCUAUAAUCAUC |
| | A-C | |
| | 33_6-6_internal_ | |
| | loop-symmetric_ | |
| | AGAUUU-CCCGAC | |
| 812 | -7_6-6_internal_ | GAUAUUGUGACAUAUCUCUGCACUCAU |
| | loop-symmetric_ | CACACAAAAGAUGCCAUGACUCUAUAG |
| | GUUCAG-AACCUG | GAUCCACAGGGAGGGGGCAUCUUAAU |
| | 5_1-1_mismatch_ | AUAUCUAACCUGUAAUCAUC |
| | A-C | |
| | 33_6-6_internal_ | |
| | loop-symmetric_ | |
| | AGAUUU-CAUGAC | |
| 813 | -7_6-6_internal_ | GAUAUUGUGACAUAUCUCUGCACUCAU |
| | loop-symmetric_ | CACACAAAAGAUGCCUUUAAUCUAUAG |
| | GUUCAG-AACGCG | GAUCCACAGGGAGGGGGCAUUUUAAU |
| | 33_6-6_internal_ | AUAUCUAACGCGUAAUCAUC |
| | loop-symmetric_ | |
| | AGAUUU-CUUUAA | |
| 814 | -7_6-6_internal_ | GAUAUUGUGACAUAUCUCUGCACUCAU |
| | loop-symmetric_ | CACACAAAAGAUGAGCGCAUUCUAUAG |
| | GUUCAG-GCGCUG | GAUCCACAGGGAGGGGGCAUUUUAAC |
| | 0_1-1_mismatch_ | AUAUCUGCGCUGUAAUCAUC |
| | A-C | |
| | 34_6-6_internal_ | |
| | loop-symmetric_ | |
| | GAUUUG-AGCGCA | |
| 815 | -7_6-6_internal_ | GAUAUUGUGACAUAUCUCUGCACUCAU |
| | loop-symmetric_ | CACACAAAAGAUGGCCUGGUUCUAUAG |
| | GUUCAG-GCCCCG | GAUCCACAGGGAGGGGGCAUUUCAAU |
| | 3_1-1_mismatch_ | AUAUCUGCCCCGUAAUCAUC |
| | A-C | |
| | 34_6-6_internal_ | |
| | loop-symmetric_ | |
| | GAUUUG-GCCUGG | |
| 816 | -7_6-6_internal_ | GAUAUUGUGACAUAUCUCUGCACUCAU |
| | loop-symmetric_ | CACACAAAAGAUGAAUAAGUUCUAUA |
| | GUUCAG-AGAUUG | GGAUCCACAGGGAGGGGGCAUUCUAA |
| | 4_1-1_mismatch_ | UAUAUCUAGAUUGUAAUCAUC |
| | A-C | |
| | 34_6-6_internal_ | |
| | loop-symmetric_ | |
| | GAUUUG-AAUAAG | |
| 817 | -7_6-6_internal_ | GAUAUUGUGACAUAUCUCUGCACUCAU |
| | loop-symmetric_ | CACACAAAAGAUGACUCGAUUCUAUAG |
| | GUUCAG-GACUCG | GAUCCACAGGGAGGGGGCAUCUUAAU |
| | 5_1-1_mismatch_ | AUAUCUGACUCGUAAUCAUC |
| | A-C | |
| | 34_6-6_internal_ | |
| | loop-symmetric_ | |
| | GAUUUG-ACUCGA | |
| 818 | -7_6-6_internal_ | GAUAUUGUGACAUAUCUCUGCACUCAU |
| | loop-symmetric_ | CACACAAAAGAUGGUCUGGUUCUAUA |
| | GUUCAG-GGUCUG | GGAUCCACAGGGAGGGGGCAUUUUAA |
| | 34_6-6_internal_ | UAUAUCUGGUCUGUAAUCAUC |
| | loop-symmetric_ | |
| | GAUUUG-GUCUGG | |
| 819 | -7_6-6_internal_ | GAUAUUGUGACAUAUCUCUGCACUCAU |
| | loop-symmetric_ | CACACAAAAGAUAGGUCGCUUCUAUAG |
| | GUUCAG-GACAUG | GAUCCACAGGGAGGGGGCAUUUUAAC |
| | 0_1-1_mismatch_ | AUAUCUGACAUGUAAUCAUC |
| | A-C | |
| | 35_6-6_internal_ | |
| | loop-symmetric_ | |
| | AUUUGC-AGGUCG | |
| 820 | -7_6-6_internal_ | GAUAUUGUGACAUAUCUCUGCACUCAU |
| | loop-symmetric_ | CACACAAAAGAUAACUGCCUUCUAUAG |
| | GUUCAG-AAUCCG | GAUCCACAGGGAGGGGGCAUUUCAAU |
| | 3_1-1_mismatch_ | AUAUCUAAUCCGUAAUCAUC |
| | A-C | |
| | 35_6-6_internal_ | |
| | loop-symmetric_ | |
| | AUUUGC-AACUGC | |
| 821 | -7_6-6_internal_ | GAUAUUGUGACAUAUCUCUGCACUCAU |
| | loop-symmetric_ | CACACAAAAGAUAUUCUACUUCUAUAG |
| | GUUCAG-ACCUCG | GAUCCACAGGGAGGGGGCAUUCUAAU |
| | 4_1-1_mismatch_ | AUAUCUACCUCGUAAUCAUC |
| | A-C | |
| | 35_6-6_internal_ | |
| | loop-symmetric_ | |
| | AUUUGC-AUUCUA | |
| 822 | -7_6-6_internal_ | GAUAUUGUGACAUAUCUCUGCACUCAU |
| | loop-symmetric_ | CACACAAAAGAUUAUGGGCUUCUAUA |
| | GUUCAG-GCCCCG | GGAUCCACAGGGAGGGGGCAUUUUAA |
| | 35_6-6_internal_ | UAUAUCUGCCCCGUAAUCAUC |
| | loop-symmetric_ | |
| | AUUUGC-UAUGGG | |
| 823 | -7_6-6_internal_ | GAUAUUGUGACAUAUCUCUGCACUCAU |
| | loop-symmetric_ | CACACAAAAGAAAGUGUUCUUCUAUA |
| | GUUCAG-AUUCCG | GGAUCCACAGGGAGGGGGCAUUUUAA |
| | 0_1-1_mismatch_ | CAUAUCUAUUCCGUAAUCAUC |
| | A-C | |
| | 36_6-6_internal_ | |
| | loop-symmetric_ | |
| | UUUGCA-AAGUGU | |
| 824 | -7_6-6_internal_ | GAUAUUGUGACAUAUCUCUGCACUCAU |
| | loop-symmetric_ | CACACAAAAGAAAAUGUUCUUCUAUA |
| | GUUCAG-GGUUGA | GGAUCCACAGGGAGGGGGCAUUUCAA |
| | 3_1-1_mismatch_ | UAUAUCUGGUUGAUAAUCAUC |
| | A-C | |
| | 36_6-6_internal_ | |
| | loop-symmetric_ | |
| | UUUGCA-AAAUGU | |
| 825 | -7_6-6_internal_ | GAUAUUGUGACAUAUCUCUGCACUCAU |
| | loop-symmetric_ | CACACAAAAGACCUCUUUCUUCUAUAG |
| | GUUCAG-AGUGUG | GAUCCACAGGGAGGGGGCAUUCUAAU |
| | 4_1-1_mismatch_ | AUAUCUAGUGUGUAAUCAUC |
| | A-C | |
| | 36_6-6_internal_ | |
| | loop-symmetric_ | |
| | UUUGCA-CCUCUU | |
| 826 | -7_6-6_internal_ | GAUAUUGUGACAUAUCUCUGCACUCAU |
| | loop-symmetric_ | CACACAAAAGAAAGGUCUCUUCUAUAG |
| | GUUCAG-AGACGA | GAUCCACAGGGAGGGGGCAUCUUAAU |
| | 5_1-1_mismatch_ | AUAUCUAGACGAUAAUCAUC |
| | A-C | |
| | 36_6-6_internal_ | |
| | loop-symmetric_ | |
| | UUUGCA-AAGGUC | |
| 827 | -7_6-6_internal_ | GAUAUUGUGACAUAUCUCUGCACUCAU |
| | loop-symmetric_ | CACACAAAAGAGAAUGUUCUUCUAUA |
| | GUUCAG-GGAUUG | GGAUCCACAGGGAGGGGGCAUUUUAA |
| | 36_6-6_internal_ | UAUAUCUGGAUUGUAAUCAUC |
| | loop-symmetric_ | |
| | UUUGCA-GAAUGU | |
| 828 | -7_6-6_internal_ | GAUAUUGUGACAUAUCUCUGCACUCAU |
| | loop-symmetric_ | CACACAAAAGCUAUACAUCUUCUAUAG |
| | GUUCAG-GAUUCA | GAUCCACAGGGAGGGGGCAUUUUAAC |
| | 0_1-1_mismatch_ | AUAUCUGAUUCAUAAUCAUC |
| | A-C | |
| | 37_6-6_internal_ | |
| | loop-symmetric_ | |
| | UUGCAU-CUAUAC | |
| 829 | -7_6-6_internal_ | GAUAUUGUGACAUAUCUCUGCACUCAU |
| | loop-symmetric_ | CACACAAAAGCCUUUUAUCUUCUAUAG |
| | GUUCAG-AGCCUA | GAUCCACAGGGAGGGGGCAUUUCAAU |
| | 3_1-1_mismatch_ | AUAUCUAGCCUAUAAUCAUC |
| | A-C | |
| | 37_6-6_internal_ | |
| | loop-symmetric_ | |
| | UUGCAU-CCUUUU | |
| 830 | -7_6-6_internal_ | GAUAUUGUGACAUAUCUCUGCACUCAU |
| | loop-symmetric_ | CACACAAAAGCUCAUUAUCUUCUAUAG |
| | GUUCAG-AGUUCG | GAUCCACAGGGAGGGGGCAUUCUAAU |
| | 4_1-1_mismatch_ | AUAUCUAGUUCGUAAUCAUC |
| | A-C | |
| | 37_6-6_internal_ | |
| | loop-symmetric_ | |
| | UUGCAU-CUCAUU | |
| 831 | -7_6-6_internal_ | GAUAUUGUGACAUAUCUCUGCACUCAU |
| | loop-symmetric_ | CACACAAAAGUAAACCAUCUUCUAUAG |
| | GUUCAG-ACUAUG | GAUCCACAGGGAGGGGGCAUCUUAAU |
| | 5_1-1_mismatch_ | AUAUCUACUAUGUAAUCAUC |
| | A-C | |
| | 37_6-6_internal_ | |
| | loop-symmetric_ | |
| | UUGCAU-UAAACC | |
| 832 | -7_6-6_internal_ | GAUAUUGUGACAUAUCUCUGCACUCAU |
| | loop-symmetric_ | CACACAAAAGCGACCUAUCUUCUAUAG |
| | GUUCAG-AGCCCG | GAUCCACAGGGAGGGGGCAUUUUAAU |
| | 37_6-6_internal_ | AUAUCUAGCCCGUAAUCAUC |
| | loop-symmetric_ | |
| | UUGCAU-CGACCU | |
| 833 | -7_6-6_internal_ | GAUAUUGUGACAUAUCUCUGCACUCAU |
| | loop-symmetric_ | CACACAAAAUUUCAUAAUCUUCUAUAG |
| | GUUCAG-GCUCUG | GAUCCACAGGGAGGGGGCAUUUUAAC |
| | 0_1-1_mismatch_ | AUAUCUGCUCUGUAAUCAUC |
| | A-C | |
| | 38_6-6_internal_ | |
| | loop-symmetric_ | |
| | UGCAUC-UUUCAU | |
| 834 | -7_6-6_internal_ | GAUAUUGUGACAUAUCUCUGCACUCAU |
| | loop-symmetric_ | CACACAAAACAAUAUAAUCUUCUAUAG |
| | GUUCAG-AGCAUA | GAUCCACAGGGAGGGGGCAUUUCAAU |
| | 3_1-1_mismatch_ | AUAUCUAGCAUAUAAUCAUC |
| | A-C | |
| | 38_6-6_internal_ | |
| | loop-symmetric_ | |
| | UGCAUC-CAAUAU | |
| 835 | -7_6-6_internal_ | GAUAUUGUGACAUAUCUCUGCACUCAU |
| | loop-symmetric_ | CACACAAAAACUUCUAAUCUUCUAUAG |
| | GUUCAG-ACACCA | GAUCCACAGGGAGGGGGCAUUCUAAU |
| | 4_1-1_mismatch_ | AUAUCUACACCAUAAUCAUC |
| | A-C | |
| | 38_6-6_internal_ | |
| | loop-symmetric_ | |
| | UGCAUC-ACUUCU | |
| 836 | -7_6-6_internal_ | GAUAUUGUGACAUAUCUCUGCACUCAU |
| | loop-symmetric_ | CACACAAAAACUAACAAUCUUCUAUAG |
| | GUUCAG-AUAAGA | GAUCCACAGGGAGGGGGCAUCUUAAU |
| | 5_1-1_mismatch_ | AUAUCUAUAAGAUAAUCAUC |
| | A-C | |
| | 38_6-6_internal_ | |
| | loop-symmetric_ | |
| | UGCAUC-ACUAAC | |
| 837 | -7_6-6_internal_ | GAUAUUGUGACAUAUCUCUGCACUCAU |
| | loop-symmetric_ | CACACAAAAUUCAACAAUCUUCUAUAG |
| | GUUCAG-ACCUUG | GAUCCACAGGGAGGGGGCAUUUUAAU |
| | 38_6-6_internal_ | AUAUCUACCUUGUAAUCAUC |
| | loop-symmetric_ | |
| | UGCAUC-UUCAAC | |
| 838 | -7_6-6_internal_ | GAUAUUGUGACAUAUCUCUGCACUCAU |
| | loop-symmetric_ | CACACAAACUCACAAAAUCUUCUAUAG |
| | GUUCAG-GAUCCA | GAUCCACAGGGAGGGGGCAUUUUAAC |
| | 0_1-1_mismatch_ | AUAUCUGAUCCAUAAUCAUC |
| | A-C | |
| | 39_6-6_internal_ | |
| | loop-symmetric_ | |
| | GCAUCU-CUCACA | |
| 839 | -7_6-6_internal_ | GAUAUUGUGACAUAUCUCUGCACUCAU |
| | loop-symmetric_ | CACACAAAUAAGUAAAAUCUUCUAUA |
| | GUUCAG-GACGCA | GGAUCCACAGGGAGGGGGCAUUUCAA |
| | 3_1-1_mismatch_ | UAUAUCUGACGCAUAAUCAUC |
| | A-C | |
| | 39_6-6_internal_ | |
| | loop-symmetric_ | |
| | GCAUCU-UAAGUA | |
| 840 | -7_6-6_internal_ | GAUAUUGUGACAUAUCUCUGCACUCAU |
| | loop-symmetric_ | CACACAAACUUCAGAAAUCUUCUAUAG |
| | GUUCAG-GGAUUG | GAUCCACAGGGAGGGGGCAUUCUAAU |
| | 4_1-1_mismatch_ | AUAUCUGGAUUGUAAUCAUC |
| | A-C | |
| | 39_6-6_internal_ | |
| | loop-symmetric_ | |
| | GCAUCU-CUUCAG | |
| 841 | -7_6-6_internal_ | GAUAUUGUGACAUAUCUCUGCACUCAU |
| | loop-symmetric_ | CACACAAAUCAAAGAAAUCUUCUAUAG |
| | GUUCAG-ACGUGA | GAUCCACAGGGAGGGGGCAUCUUAAU |
| | 5_1-1_mismatch_ | AUAUCUACGUGAUAAUCAUC |
| | A-C | |
| | 39_6-6_internal_ | |
| | loop-symmetric_ | |
| | GCAUCU-UCAAAG | |
| 842 | -7_6-6_internal_ | GAUAUUGUGACAUAUCUCUGCACUCAU |
| | loop-symmetric_ | CACACAAAUCACUGAAAUCUUCUAUAG |
| | GUUCAG-ACCGCA | GAUCCACAGGGAGGGGGCAUUUUAAU |
| | 39_6-6_internal_ | AUAUCUACCGCAUAAUCAUC |
| | loop-symmetric_ | |
| | GCAUCU-UCACUG | |
| 843 | -7_6-6_internal_ | GAUAUUGUGACAUAUCUCUGCACUCAU |
| | loop-symmetric_ | CACACAACCUCCACAAAUCUUCUAUAG |
| | GUUCAG-GCCCAA | GAUCCACAGGGAGGGGGCAUUUUAAC |
| | 0_1-1_mismatch_ | AUAUCUGCCCAAUAAUCAUC |
| | A-C | |
| | 40_6-6_internal_ | |
| | loop-symmetric_ | |
| | CAUCUU-CCUCCA | |
| 844 | -7_6-6_internal_ | GAUAUUGUGACAUAUCUCUGCACUCAU |
| | loop-symmetric_ | CACACAACGUCAUCAAAUCUUCUAUAG |
| | GUUCAG-AGAUUG | GAUCCACAGGGAGGGGGCAUUUCAAU |
| | 3_1-1_mismatch_ | AUAUCUAGAUUGUAAUCAUC |
| | A-C | |
| | 40_6-6_internal_ | |
| | loop-symmetric_ | |
| | CAUCUU-CGUCAU | |
| 845 | -7_6-6_internal_ | GAUAUUGUGACAUAUCUCUGCACUCAU |
| | loop-symmetric_ | CACACAAUCUAAACAAAUCUUCUAUAG |
| | GUUCAG-AGUUCG | GAUCCACAGGGAGGGGGCAUUCUAAU |
| | 4_1-1_mismatch_ | AUAUCUAGUUCGUAAUCAUC |
| | A-C | |
| | 40_6-6_internal_ | |
| | loop-symmetric_ | |
| | CAUCUU-UCUAAA | |
| 846 | -7_6-6_internal_ | GAUAUUGUGACAUAUCUCUGCACUCAU |
| | loop-symmetric_ | CACACAUCCUAGGCAAAUCUUCUAUAG |
| | GUUCAG-GAAAGA | GAUCCACAGGGAGGGGGCAUUUUAAC |
| | 0_1-1_mismatch_ | AUAUCUGAAAGAUAAUCAUC |
| | A-C | |
| | 41_6-6_internal_ | |
| | loop-symmetric_ | |
| | AUCUUU-UCCUAG | |
| 847 | -7_6-6_internal_ | GAUAUUGUGACAUAUCUCUGCACUCAU |
| | loop-symmetric_ | CACACAUCAUGCGCAAAUCUUCUAUAG |
| | GUUCAG-AGCGUG | GAUCCACAGGGAGGGGGCAUUUCAAU |
| | 3_1-1_mismatch_ | AUAUCUAGCGUGUAAUCAUC |
| | A-C | |
| | 41_6-6_internal_ | |
| | loop-symmetric_ | |
| | AUCUUU-UCAUGC | |
| 848 | -7_6-6_internal_ | GAUAUUGUGACAUAUCUCUGCACUCAU |
| | loop-symmetric_ | CACACACGCCCAGCAAAUCUUCUAUAG |
| | GUUCAG-AACUCG | GAUCCACAGGGAGGGGGCAUUCUAAU |
| | 4_1-1_mismatch_ | AUAUCUAACUCGUAAUCAUC |
| | A-C | |
| | 41_6-6_internal_ | |
| | loop-symmetric_ | |
| | AUCUUU-CGCCCA | |
| 849 | -7_6-6_internal_ | GAUAUUGUGACAUAUCUCUGCACUCAU |
| | loop-symmetric_ | CACACACUAUCGGCAAAUCUUCUAUAG |
| | GUUCAG-GCUCCA | GAUCCACAGGGAGGGGGCAUCUUAAU |
| | 5_1-1_mismatch_ | AUAUCUGCUCCAUAAUCAUC |
| | A-C | |
| | 41_6-6_internal_ | |
| | loop-symmetric_ | |
| | AUCUUU-CUAUCG | |
| 850 | -7_6-6_internal_ | GAUAUUGUGACAUAUCUCUGCACUCAU |
| | loop-symmetric_ | CACACACCGUUCGCAAAUCUUCUAUAG |
| | GUUCAG-GACUCG | GAUCCACAGGGAGGGGGCAUUUUAAU |
| | 41_6-6_internal_ | AUAUCUGACUCGUAAUCAUC |
| | loop-symmetric_ | |
| | AUCUUU-CCGUUC | |
| 851 | -7_6-6_internal_ | GAUAUUGUGACAUAUCUCUGCACUCAU |
| | loop-symmetric_ | CACACCCGCUCUGCAAAUCUUCUAUAG |
| | GUUCAG-ACGUCG | GAUCCACAGGGAGGGGGCAUUUUAAC |
| | 0_1-1_mismatch_ | AUAUCUACGUCGUAAUCAUC |
| | A-C | |
| | 42_6-6_internal_ | |
| | loop-symmetric_ | |
| | UCUUUU-CCGCUC | |
| 852 | -7_6-6_internal_ | GAUAUUGUGACAUAUCUCUGCACUCAU |
| | loop-symmetric_ | CACACUGCGUUUGCAAAUCUUCUAUAG |
| | GUUCAG-GGUUUG | GAUCCACAGGGAGGGGGCAUUUCAAU |
| | 3_1-1_mismatch_ | AUAUCUGGUUUGUAAUCAUC |
| | A-C | |
| | 42_6-6_internal_ | |
| | loop-symmetric_ | |
| | UCUUUU-UGCGUU | |
| 853 | -7_6-6_internal_ | GAUAUUGUGACAUAUCUCUGCACUCAU |
| | loop-symmetric_ | CACACUUACCCUGCAAAUCUUCUAUAG |
| | GUUCAG-GAAGUG | GAUCCACAGGGAGGGGGCAUUCUAAU |
| | 4_1-1_mismatch_ | AUAUCUGAAGUGUAAUCAUC |
| | A-C | |
| | 42_6-6_internal_ | |
| | loop-symmetric_ | |
| | UCUUUU-UUACCC | |
| 854 | -7_6-6_internal_ | GAUAUUGUGACAUAUCUCUGCACUCAU |
| | loop-symmetric_ | CACACCUAUACUGCAAAUCUUCUAUAG |
| | GUUCAG-GACUAA | GAUCCACAGGGAGGGGGCAUCUUAAU |
| | 5_1-1_mismatch_ | AUAUCUGACUAAUAAUCAUC |
| | A-C | |
| | 42_6-6_internal_ | |
| | loop-symmetric_ | |
| | UCUUUU-CUAUAC | |
| 855 | -7_6-6_internal_ | GAUAUUGUGACAUAUCUCUGCACUCAU |
| | loop-symmetric_ | CACACCAUCUCUGCAAAUCUUCUAUAG |
| | GUUCAG-AGUCUG | GAUCCACAGGGAGGGGGCAUUUUAAU |
| | 42_6-6_internal_ | AUAUCUAGUCUGUAAUCAUC |
| | loop-symmetric_ | |
| | UCUUUU-CAUCUC | |
| 856 | -7_6-6_internal_ | GAUAUUGUGACAUAUCUCUGCACUCAU |
| | loop-symmetric_ | CACAAGCUCAAUGCAAAUCUUCUAUAG |
| | GUUCAG-GCAGUA | GAUCCACAGGGAGGGGGCAUUUUAAC |
| | 0_1-1_mismatch_ | AUAUCUGCAGUAUAAUCAUC |
| | A-C | |
| | 43_6-6_internal_ | |
| | loop-symmetric_ | |
| | CUUUUG-AGCUCA | |
| 857 | -7_6-6_internal_ | GAUAUUGUGACAUAUCUCUGCACUCAU |
| | loop-symmetric_ | CACAGCCCAAAUGCAAAUCUUCUAUAG |
| | GUUCAG-GCAUCG | GAUCCACAGGGAGGGGGCAUUUCAAU |
| | 3_1-1_mismatch_ | AUAUCUGCAUCGUAAUCAUC |
| | A-C | |
| | 43_6-6_internal_ | |
| | loop-symmetric_ | |
| | CUUUUG-GCCCAA | |
| 858 | -7_6-6_internal_ | GAUAUUGUGACAUAUCUCUGCACUCAU |
| | loop-symmetric_ | CACAGUCAUAAUGCAAAUCUUCUAUAG |
| | GUUCAG-GAUUUG | GAUCCACAGGGAGGGGGCAUUCUAAU |
| | 4_1-1_mismatch_ | AUAUCUGAUUUGUAAUCAUC |
| | A-C | |
| | 43_6-6_internal_ | |
| | loop-symmetric_ | |
| | CUUUUG-GUCAUA | |
| 859 | -7_6-6_internal_ | GAUAUUGUGACAUAUCUCUGCACUCAU |
| | loop-symmetric_ | CACAAGUGCUAUGCAAAUCUUCUAUAG |
| | GUUCAG-GAUCUA | GAUCCACAGGGAGGGGGCAUCUUAAU |
| | 5_1-1_mismatch_ | AUAUCUGAUCUAUAAUCAUC |
| | A-C | |
| | 43_6-6_internal_ | |
| | loop-symmetric_ | |
| | CUUUUG-AGUGCU | |
| 860 | -7_6-6_internal_ | GAUAUUGUGACAUAUCUCUGCACUCAU |
| | loop-symmetric_ | CACAACGCUAAUGCAAAUCUUCUAUAG |
| | GUUCAG-GGUUAA | GAUCCACAGGGAGGGGGCAUUUUAAU |
| | 43_6-6_internal_ | AUAUCUGGUUAAUAAUCAUC |
| | loop-symmetric_ | |
| | CUUUUG-ACGCUA | |
| 861 | -7_6-6_internal_ | GAUAUUGUGACAUAUCUCUGCACUCAU |
| | loop-symmetric_ | CACCUGGCUGAUGCAAAUCUUCUAUAG |
| | GUUCAG-AGCUUG | GAUCCACAGGGAGGGGGCAUUUUAAC |
| | 0_1-1_mismatch_ | AUAUCUAGCUUGUAAUCAUC |
| | A-C | |
| | 44_6-6_internal_ | |
| | loop-symmetric_ | |
| | UUUUGU-CUGGCU | |
| 862 | -7_6-6_internal_ | GAUAUUGUGACAUAUCUCUGCACUCAU |
| | loop-symmetric_ | CACCUUUGCGAUGCAAAUCUUCUAUAG |
| | GUUCAG-GGUAGA | GAUCCACAGGGAGGGGGCAUUUCAAU |
| | 3_1-1_mismatch_ | AUAUCUGGUAGAUAAUCAUC |
| | A-C | |
| | 44_6-6_internal_ | |
| | loop-symmetric_ | |
| | UUUUGU-CUUUGC | |
| 863 | -7_6-6_internal_ | GAUAUUGUGACAUAUCUCUGCACUCAU |
| | loop-symmetric_ | CACCCCGCCGAUGCAAAUCUUCUAUAG |
| | GUUCAG-GACUAA | GAUCCACAGGGAGGGGGCAUUCUAAU |
| | 4_1-1_mismatch_ | AUAUCUGACUAAUAAUCAUC |
| | A-C | |
| | 44_6-6_internal_ | |
| | loop-symmetric_ | |
| | UUUUGU-CCCGCC | |
| 864 | -7_6-6_internal_ | GAUAUUGUGACAUAUCUCUGCACUCAU |
| | loop-symmetric_ | CACUGGUGCGAUGCAAAUCUUCUAUAG |
| | GUUCAG-GCGCUG | GAUCCACAGGGAGGGGGCAUCUUAAU |
| | 5_1-1_mismatch_ | AUAUCUGCGCUGUAAUCAUC |
| | A-C | |
| | 44_6-6_internal_ | |
| | loop-symmetric_ | |
| | UUUUGU-UGGUGC | |
| 865 | -7_6-6_internal_ | GAUAUUGUGACAUAUCUCUGCACUCAU |
| | loop-symmetric_ | CACUAAUUCGAUGCAAAUCUUCUAUAG |
| | GUUCAG-GACAAA | GAUCCACAGGGAGGGGGCAUUUUAAU |
| | 44_6-6_internal_ | AUAUCUGACAAAUAAUCAUC |
| | loop-symmetric_ | |
| | UUUUGU-UAAUUC | |
| 866 | -6_6-6_internal_ | GAUAUUGUGACAUAUCUCUGCACUCAU |
| | loop-symmetric_ | CACACAAAAGAUGCAAAUCUUCUAUAG |
| | UUCAGA-AUAAAU | GAUCCACAGGGAGGGGGCAUUUUAAC |
| | 0_1-1_mismatch_ | AUAUCAUAAAUCUAAUCAUC |
| | A-C | |
| 867 | -6_6-6_internal_ | GAUAUUGUGACAUAUCUCUGCACUCAU |
| | loop-symmetric_ | CACACAAAAGAUGCAAAUCUUCUAUAG |
| | UUCAGA-AAGUGC | GAUCCACAGGGAGGGGGCAUUUCAAU |
| | 3_1-1_mismatch_ | AUAUCAAGUGCCUAAUCAUC |
| | A-C | |
| 868 | -6_6-6_internal_ | GAUAUUGUGACAUAUCUCUGCACUCAU |
| | loop-symmetric_ | CACACAAAAGAUGCAAAUCUUCUAUAG |
| | UUCAGA-AUACCC | GAUCCACAGGGAGGGGGCAUUCUAAU |
| | 4_1-1_mismatch_ | AUAUCAUACCCCUAAUCAUC |
| | A-C | |
| 869 | -6_6-6_internal_ | GAUAUUGUGACAUAUCUCUGCACUCAU |
| | loop-symmetric_ | CACACAAAAGAUGCAAAUCUUCUAUAG |
| | UUCAGA-AAUCCC | GAUCCACAGGGAGGGGGCAUCUUAAU |
| | 5_1-1_mismatch_ | AUAUCAAUCCCCUAAUCAUC |
| | A-C | |
| 870 | -6_6-6_internal_ | GAUAUUGUGACAUAUCUCUGCACUCAU |
| | loop-symmetric_ | CACACAAAAGAUGCAAAUCUUCUAUAG |
| | UUCAGA-CAUACC | GAUCCACAGGGAGGGGGCAUUUUAAU |
| | | AUAUCCAUACCCUAAUCAUC |
| 871 | -6_6-6_internal_ | GAUAUUGUGACAUAUCUCUGCACUCAU |
| | loop-symmetric_ | CACACAAAAGAUGCAAAUCUUCUAUAG |
| | UUCAGA-GUUCAC | CUGUGUCAGGGAGGGGGCAUUUUAAC |
| | 0_1-1_mismatch_ | AUAUCGUUCACCUAAUCAUC |
| | A-C | |
| | 20_6-6_internal_ | |
| | loop-symmetric_ | |
| | UGGAUC-CUGUGU | |
| 872 | -6_6-6_internal_ | GAUAUUGUGACAUAUCUCUGCACUCAU |
| | loop-symmetric_ | CACACAAAAGAUGCAAAUCUUCUAUAG |
| | UUCAGA-ACCUUC | ACAUAUCAGGGAGGGGGCAUUUCAAU |
| | 3_1-1_mismatch_ | AUAUCACCUUCCUAAUCAUC |
| | A-C | |
| | 20_6-6_internal_ | |
| | loop-symmetric_ | |
| | UGGAUC-ACAUAU | |
| 873 | -6_6-6_internal_ | GAUAUUGUGACAUAUCUCUGCACUCAU |
| | loop-symmetric_ | CACACAAAAGAUGCAAAUCUUCUAUAG |
| | UUCAGA-CAUUAU | AGCGUUCAGGGAGGGGGCAUUCUAAU |
| | 4_1-1_mismatch_ | AUAUCCAUUAUCUAAUCAUC |
| | A-C | |
| | 20_6-6_internal_ | |
| | loop-symmetric_ | |
| | UGGAUC-AGCGUU | |
| 874 | -6_6-6_internal_ | GAUAUUGUGACAUAUCUCUGCACUCAU |
| | loop-symmetric_ | CACACAAAAGAUGCAAAUCUUCUAUAG |
| | UUCAGA-AGGCUU | UUGUGUCAGGGAGGGGGCAUCUUAAU |
| | 5_1-1_mismatch_ | AUAUCAGGCUUCUAAUCAUC |
| | A-C | |
| | 20_6-6_internal_ | |
| | loop-symmetric_ | |
| | UGGAUC-UUGUGU | |
| 875 | -6_6-6_internal_ | GAUAUUGUGACAUAUCUCUGCACUCAU |
| | loop-symmetric_ | CACACAAAAGAUGCAAAUCUUCUAUAG |
| | UUCAGA-GGCGCU | AACGAUCAGGGAGGGGGCAUUUUAAU |
| | 20_6-6_internal_ | AUAUCGGCGCUCUAAUCAUC |
| | loop-symmetric_ | |
| | UGGAUC-AACGAU | |
| 876 | -6_6-6_internal_ | GAUAUUGUGACAUAUCUCUGCACUCAU |
| | loop-symmetric_ | CACACAAAAGAUGCAAAUCUUCUAUAU |
| | UUCAGA-GUUCAU | CACGGACAGGGAGGGGGCAUUUUAAC |
| | 0_1-1_mismatch_ | AUAUCGUUCAUCUAAUCAUC |
| | A-C | |
| | 21_6-6_internal_ | |
| | loop-symmetric_ | |
| | GGAUCC-UCACGG | |
| 877 | -6_6-6_internal_ | GAUAUUGUGACAUAUCUCUGCACUCAU |
| | loop-symmetric_ | CACACAAAAGAUGCAAAUCUUCUAUAA |
| | UUCAGA-AAGUCC | GCAGGACAGGGAGGGGGCAUUUCAAU |
| | 3_1-1_mismatch_ | AUAUCAAGUCCCUAAUCAUC |
| | A-C | |
| | 21_6-6_internal_ | |
| | loop-symmetric_ | |
| | GGAUCC-AGCAGG | |
| 878 | -6_6-6_internal_ | GAUAUUGUGACAUAUCUCUGCACUCAU |
| | loop-symmetric_ | CACACAAAAGAUGCAAAUCUUCUAUAC |
| | UUCAGA-CACUUU | AACAAACAGGGAGGGGGCAUUCUAAU |
| | 4_1-1_mismatch_ | AUAUCCACUUUCUAAUCAUC |
| | A-C | |
| | 21_6-6_internal_ | |
| | loop-symmetric_ | |
| | GGAUCC-CAACAA | |
| 879 | -6_6-6_internal_ | GAUAUUGUGACAUAUCUCUGCACUCAU |
| | loop-symmetric_ | CACACAAAAGAUGCAAAUCUUCUAUAC |
| | UUCAGA-AUAUCC | UCAGAACAGGGAGGGGGCAUCUUAAU |
| | 5_1-1_mismatch_ | AUAUCAUAUCCCUAAUCAUC |
| | A-C | |
| | 21_6-6_internal_ | |
| | loop-symmetric_ | |
| | GGAUCC-CUCAGA | |
| 880 | -6_6-6_internal_ | GAUAUUGUGACAUAUCUCUGCACUCAU |
| | loop-symmetric_ | CACACAAAAGAUGCAAAUCUUCUAUAC |
| | UUCAGA-ACCUUC | UCGAGACAGGGAGGGGGCAUUUUAAU |
| | 21_6-6_internal_ | AUAUCACCUUCCUAAUCAUC |
| | loop-symmetric_ | |
| | GGAUCC-CUCGAG | |
| 881 | -6_6-6_internal_ | GAUAUUGUGACAUAUCUCUGCACUCAU |
| | loop-symmetric_ | CACACAAAAGAUGCAAAUCUUCUAUUU |
| | UUCAGA-ACAAUC | CAAACACAGGGAGGGGGCAUUUUAAC |
| | 0_1-1_mismatch_ | AUAUCACAAUCCUAAUCAUC |
| | A-C | |
| | 22_6-6_internal_ | |
| | loop-symmetric_ | |
| | GAUCCU-UUCAAA | |
| 882 | -6_6-6_internal_ | GAUAUUGUGACAUAUCUCUGCACUCAU |
| | loop-symmetric_ | CACACAAAAGAUGCAAAUCUUCUAUCA |
| | UUCAGA-GUAACU | CUAGCACAGGGAGGGGGCAUUUCAAU |
| | 3_1-1_mismatch_ | AUAUCGUAACUCUAAUCAUC |
| | A-C | |
| | 22_6-6_internal_ | |
| | loop-symmetric_ | |
| | GAUCCU-CACUAG | |
| 883 | -6_6-6_internal_ | GAUAUUGUGACAUAUCUCUGCACUCAU |
| | loop-symmetric_ | CACACAAAAGAUGCAAAUCUUCUAUUU |
| | UUCAGA-CACAUU | CUGACACAGGGAGGGGGCAUUCUAAU |
| | 4_1-1_mismatch_ | AUAUCCACAUUCUAAUCAUC |
| | A-C | |
| | 22_6-6_internal_ | |
| | loop-symmetric_ | |
| | GAUCCU-UUCUGA | |
| 884 | -6_6-6_internal_ | GAUAUUGUGACAUAUCUCUGCACUCAU |
| | loop-symmetric_ | CACACAAAAGAUGCAAAUCUUCUAUCC |
| | UUCAGA-GGUUAU | UCCACACAGGGAGGGGGCAUCUUAAUA |
| | 5_1-1_mismatch_ | UAUCGGUUAUCUAAUCAUC |
| | A-C | |
| | 22_6-6_internal_ | |
| | loop-symmetric_ | |
| | GAUCCU-CCUCCA | |
| 885 | -6_6-6_internal_ | GAUAUUGUGACAUAUCUCUGCACUCAU |
| | loop-symmetric_ | CACACAAAAGAUGCAAAUCUUCUAUCC |
| | UUCAGA-GGAACC | AACGCACAGGGAGGGGGCAUUUUAAU |
| | 22_6-6_internal_ | AUAUCGGAACCCUAAUCAUC |
| | loop-symmetric_ | |
| | GAUCCU-CCAACG | |
| 886 | -6_6-6_internal_ | GAUAUUGUGACAUAUCUCUGCACUCAU |
| | loop-symmetric_ | CACACAAAAGAUGCAAAUCUUCUAGUC |
| | UUCAGA-AGGCUC | CAGCCACAGGGAGGGGGCAUUUUAACA |
| | 0_1-1_mismatch_ | UAUCAGGCUCCUAAUCAUC |
| | A-C | |
| | 23_6-6_internal_ | |
| | loop-symmetric_ | |
| | AUCCUA-GUCCAG | |
| 887 | -6_6-6_internal_ | GAUAUUGUGACAUAUCUCUGCACUCAU |
| | loop-symmetric_ | CACACAAAAGAUGCAAAUCUUCUAAAU |
| | UUCAGA-GGCCCU | UUCCCACAGGGAGGGGGCAUUUCAAUA |
| | 3_1-1_mismatch_ | UAUCGGCCCUCUAAUCAUC |
| | A-C | |
| | 23_6-6_internal_ | |
| | loop-symmetric_ | |
| | AUCCUA-AAUUUC | |
| 888 | -6_6-6_internal_ | GAUAUUGUGACAUAUCUCUGCACUCAU |
| | loop-symmetric_ | CACACAAAAGAUGCAAAUCUUCUAGUA |
| | UUCAGA-AGGCCU | UCCCCACAGGGAGGGGGCAUUCUAAUA |
| | 4_1-1_mismatch_ | UAUCAGGCCUCUAAUCAUC |
| | A-C | |
| | 23_6-6_internal_ | |
| | loop-symmetric_ | |
| | AUCCUA-GUAUCC | |
| 889 | -6_6-6_internal_ | GAUAUUGUGACAUAUCUCUGCACUCAU |
| | loop-symmetric_ | CACACAAAAGAUGCAAAUCUUCUAAAA |
| | UUCAGA-GGCCUC | AUACCACAGGGAGGGGGCAUCUUAAU |
| | 5_1-1_mismatch_ | AUAUCGGCCUCCUAAUCAUC |
| | A-C | |
| | 23_6-6_internal_ | |
| | loop-symmetric_ | |
| | AUCCUA-AAAAUA | |
| 890 | -6_6-6_internal_ | GAUAUUGUGACAUAUCUCUGCACUCAU |
| | loop-symmetric_ | CACACAAAAGAUGCAAAUCUUCUAAGU |
| | UUCAGA-AGACCC | AAACCACAGGGAGGGGGCAUUUUAAU |
| | 23_6-6_internal_ | AUAUCAGACCCCUAAUCAUC |
| | loop-symmetric_ | |
| | AUCCUA-AGUAAA | |
| 891 | -6_6-6_internal_ | GAUAUUGUGACAUAUCUCUGCACUCAU |
| | loop-symmetric_ | CACACAAAAGAUGCAAAUCUUCUCUUU |
| | UUCAGA-AUACUC | UUUCCACAGGGAGGGGGCAUUUUAAC |
| | 0_1-1_mismatch_ | AUAUCAUACUCCUAAUCAUC |
| | A-C | |
| | 24_6-6_internal_ | |
| | loop-symmetric_ | |
| | UCCUAU-CUUUUU | |
| 892 | -6_6-6_internal_ | GAUAUUGUGACAUAUCUCUGCACUCAU |
| | loop-symmetric_ | CACACAAAAGAUGCAAAUCUUCUUGAC |
| | UUCAGA-AAUCGC | CUUCCACAGGGAGGGGGCAUUUCAAUA |
| | 3_1-1_mismatch_ | UAUCAAUCGCCUAAUCAUC |
| | A-C | |
| | 24_6-6_internal_ | |
| | loop-symmetric_ | |
| | UCCUAU-UGACCU | |
| 893 | -6_6-6_internal_ | GAUAUUGUGACAUAUCUCUGCACUCAU |
| | loop-symmetric_ | CACACAAAAGAUGCAAAUCUUCUCCGU |
| | UUCAGA-AAUACU | ACUCCACAGGGAGGGGGCAUUCUAAUA |
| | 4_1-1_mismatch_ | UAUCAAUACUCUAAUCAUC |
| | A-C | |
| | 24_6-6_internal_ | |
| | loop-symmetric_ | |
| | UCCUAU-CCGUAC | |
| 894 | -6_6-6_internal_ | GAUAUUGUGACAUAUCUCUGCACUCAU |
| | loop-symmetric_ | CACACAAAAGAUGCAAAUCUUCUCCUU |
| | UUCAGA-AAGCUC | UCUCCACAGGGAGGGGGCAUCUUAAUA |
| | 5_1-1_mismatch_ | UAUCAAGCUCCUAAUCAUC |
| | A-C | |
| | 24_6-6_internal_ | |
| | loop-symmetric_ | |
| | UCCUAU-CCUUUC | |
| 895 | -6_6-6_internal_ | GAUAUUGUGACAUAUCUCUGCACUCAU |
| | loop-symmetric_ | CACACAAAAGAUGCAAAUCUUCUCGAC |
| | UUCAGA-GACUUC | GCUCCACAGGGAGGGGGCAUUUUAAU |
| | 24_6-6_internal_ | AUAUCGACUUCCUAAUCAUC |
| | loop-symmetric_ | |
| | UCCUAU-CGACGC | |
| 896 | -6_6-6_internal_ | GAUAUUGUGACAUAUCUCUGCACUCAU |
| | loop-symmetric_ | CACACAAAAGAUGCAAAUCUUCAACGC |
| | UUCAGA-CAAAGU | CAUCCACAGGGAGGGGGCAUUUUAACA |
| | 0_1-1_mismatch_ | UAUCCAAAGUCUAAUCAUC |
| | A-C | |
| | 25_6-6_internal_ | |
| | loop-symmetric_ | |
| | CCUAUA-AACGCC | |
| 897 | -6_6-6_internal_ | GAUAUUGUGACAUAUCUCUGCACUCAU |
| | loop-symmetric_ | CACACAAAAGAUGCAAAUCUUCCAACG |
| | UUCAGA-ACCUUU | CAUCCACAGGGAGGGGGCAUUUCAAUA |
| | 3_1-1_mismatch_ | UAUCACCUUUCUAAUCAUC |
| | A-C | |
| | 25_6-6_internal_ | |
| | loop-symmetric_ | |
| | CCUAUA-CAACGC | |
| 898 | -6_6-6_internal_ | GAUAUUGUGACAUAUCUCUGCACUCAU |
| | loop-symmetric_ | CACACAAAAGAUGCAAAUCUUCAGCGC |
| | UUCAGA-AGCAUC | AAUCCACAGGGAGGGGGCAUUCUAAU |
| | 4_1-1_mismatch_ | AUAUCAGCAUCCUAAUCAUC |
| | A-C | |
| | 25_6-6_internal_ | |
| | loop-symmetric_ | |
| | CCUAUA-AGCGCA | |
| 899 | -6_6-6_internal_ | GAUAUUGUGACAUAUCUCUGCACUCAU |
| | loop-symmetric_ | CACACAAAAGAUGCAAAUCUUCGUGUC |
| | UUCAGA-CAAAUU | AAUCCACAGGGAGGGGGCAUCUUAAU |
| | 5_1-1_mismatch_ | AUAUCCAAAUUCUAAUCAUC |
| | A-C | |
| | 25_6-6_internal_ | |
| | loop-symmetric_ | |
| | CCUAUA-GUGUCA | |
| 900 | -6_6-6_internal_ | GAUAUUGUGACAUAUCUCUGCACUCAU |
| | loop-symmetric_ | CACACAAAAGAUGCAAAUCUUCGAAUA |
| | UUCAGA-AGCUUU | AAUCCACAGGGAGGGGGCAUUUUAAU |
| | 25_6-6_internal_ | AUAUCAGCUUUCUAAUCAUC |
| | loop-symmetric_ | |
| | CCUAUA-GAAUAA | |
| 901 | -6_6-6_internal_ | GAUAUUGUGACAUAUCUCUGCACUCAU |
| | loop-symmetric_ | CACACAAAAGAUGCAAAUCUUACGAAA |
| | UUCAGA-GGAAUC | GAUCCACAGGGAGGGGGCAUUUUAAC |
| | 0_1-1_mismatch_ | AUAUCGGAAUCCUAAUCAUC |
| | A-C | |
| | 26_6-6_internal_ | |
| | loop-symmetric_ | |
| | CUAUAG-ACGAAA | |
| 902 | -6_6-6_internal_ | GAUAUUGUGACAUAUCUCUGCACUCAU |
| | loop-symmetric_ | CACACAAAAGAUGCAAAUCUUGGUCCU |
| | UUCAGA-GUAAUU | GAUCCACAGGGAGGGGGCAUUUCAAU |
| | 3_1-1_mismatch_ | AUAUCGUAAUUCUAAUCAUC |
| | A-C | |
| | 26_6-6_internal_ | |
| | loop-symmetric_ | |
| | CUAUAG-GGUCCU | |
| 903 | -6_6-6_internal_ | GAUAUUGUGACAUAUCUCUGCACUCAU |
| | loop-symmetric_ | CACACAAAAGAUGCAAAUCUUGCACCU |
| | UUCAGA-GACACC | GAUCCACAGGGAGGGGGCAUUCUAAU |
| | 4_1-1_mismatch_ | AUAUCGACACCCUAAUCAUC |
| | A-C | |
| | 26_6-6_internal_ | |
| | loop-symmetric_ | |
| | CUAUAG-GCACCU | |
| 904 | -6_6-6_internal_ | GAUAUUGUGACAUAUCUCUGCACUCAU |
| | loop-symmetric_ | CACACAAAAGAUGCAAAUCUUAGGAA |
| | UUCAGA-AGGUAU | AGAUCCACAGGGAGGGGGCAUCUUAA |
| | 5_1-1_mismatch_ | UAUAUCAGGUAUCUAAUCAUC |
| | A-C | |
| | 26_6-6_internal_ | |
| | loop-symmetric_ | |
| | CUAUAG-AGGAAA | |
| 905 | -6_6-6_internal_ | GAUAUUGUGACAUAUCUCUGCACUCAU |
| | loop-symmetric_ | CACACAAAAGAUGCAAAUCUUACGACA |
| | UUCAGA-GUACAC | GAUCCACAGGGAGGGGGCAUUUUAAU |
| | 26_6-6_internal_ | AUAUCGUACACCUAAUCAUC |
| | loop-symmetric_ | |
| | CUAUAG-ACGACA | |
| 906 | -6_6-6_internal_ | GAUAUUGUGACAUAUCUCUGCACUCAU |
| | loop-symmetric_ | CACACAAAAGAUGCAAAUCUGUGGGCG |
| | UUCAGA-AGCUCC | GAUCCACAGGGAGGGGGCAUUUUAAC |
| | 0_1-1_mismatch_ | AUAUCAGCUCCCUAAUCAUC |
| | A-C | |
| | 27_6-6_internal_ | |
| | loop-symmetric_ | |
| | UAUAGA-GUGGGC | |
| 907 | -6_6-6_internal_ | GAUAUUGUGACAUAUCUCUGCACUCAU |
| | loop-symmetric_ | CACACAAAAGAUGCAAAUCUCAUCGCG |
| | UUCAGA-GGUACC | GAUCCACAGGGAGGGGGCAUUUCAAU |
| | 3_1-1_mismatch_ | AUAUCGGUACCCUAAUCAUC |
| | A-C | |
| | 27_6-6_internal_ | |
| | loop-symmetric_ | |
| | UAUAGA-CAUCGC | |
| 908 | -6_6-6_internal_ | GAUAUUGUGACAUAUCUCUGCACUCAU |
| | loop-symmetric_ | CACACAAAAGAUGCAAAUCUAGCCUUG |
| | UUCAGA-GGGCUC | GAUCCACAGGGAGGGGGCAUUCUAAU |
| | 4_1-1_mismatch_ | AUAUCGGGCUCCUAAUCAUC |
| | A-C | |
| | 27_6-6_internal_ | |
| | loop-symmetric_ | |
| | UAUAGA-AGCCUU | |
| 909 | -6_6-6_internal_ | GAUAUUGUGACAUAUCUCUGCACUCAU |
| | loop-symmetric_ | CACACAAAAGAUGCAAAUCUGAACACG |
| | UUCAGA-AAGCUU | GAUCCACAGGGAGGGGGCAUCUUAAU |
| | 5_1-1_mismatch_ | AUAUCAAGCUUCUAAUCAUC |
| | A-C | |
| | 27_6-6_internal_ | |
| | loop-symmetric_ | |
| | UAUAGA-GAACAC | |
| 910 | -6_6-6_internal_ | GAUAUUGUGACAUAUCUCUGCACUCAU |
| | loop-symmetric_ | CACACAAAAGAUGCAAAUCUGCGCGUG |
| | UUCAGA-GCCUUC | GAUCCACAGGGAGGGGGCAUUUUAAU |
| | 27_6-6_internal_ | AUAUCGCCUUCCUAAUCAUC |
| | loop-symmetric_ | |
| | UAUAGA-GCGCGU | |
| 911 | -6_6-6_internal_ | GAUAUUGUGACAUAUCUCUGCACUCAU |
| | loop-symmetric_ | CACACAAAAGAUGCAAAUCCUGGAGAG |
| | UUCAGA-AAUUCU | GAUCCACAGGGAGGGGGCAUUUUAAC |
| | 0_1-1_mismatch_ | AUAUCAAUUCUCUAAUCAUC |
| | A-C | |
| | 28_6-6_internal_ | |
| | loop-symmetric_ | |
| | AUAGAA-CUGGAG | |
| 912 | -6_6-6_internal_ | GAUAUUGUGACAUAUCUCUGCACUCAU |
| | loop-symmetric_ | CACACAAAAGAUGCAAAUCGCAAACAG |
| | UUCAGA-GUAUCU | GAUCCACAGGGAGGGGGCAUUUCAAU |
| | 3_1-1_mismatch_ | AUAUCGUAUCUCUAAUCAUC |
| | A-C | |
| | 28_6-6_internal_ | |
| | loop-symmetric_ | |
| | AUAGAA-GCAAAC | |
| 913 | -6_6-6_internal_ | GAUAUUGUGACAUAUCUCUGCACUCAU |
| | loop-symmetric_ | CACACAAAAGAUGCAAAUCGGUGCGAG |
| | UUCAGA-AAACUU | GAUCCACAGGGAGGGGGCAUUCUAAU |
| | 4_1-1_mismatch_ | AUAUCAAACUUCUAAUCAUC |
| | A-C | |
| | 28_6-6_internal_ | |
| | loop-symmetric_ | |
| | AUAGAA-GGUGCG | |
| 914 | -6_6-6_internal_ | GAUAUUGUGACAUAUCUCUGCACUCAU |
| | loop-symmetric_ | CACACAAAAGAUGCAAAUCGAGGAAA |
| | UUCAGA-AGGCCU | GGAUCCACAGGGAGGGGGCAUCUUAA |
| | 5_1-1_mismatch_ | UAUAUCAGGCCUCUAAUCAUC |
| | A-C | |
| | 28_6-6_internal_ | |
| | loop-symmetric_ | |
| | AUAGAA-GAGGAA | |
| 915 | -6_6-6_internal_ | GAUAUUGUGACAUAUCUCUGCACUCAU |
| | loop-symmetric_ | CACACAAAAGAUGCAAAUCCGGAAGAG |
| | UUCAGA-AAACCC | GAUCCACAGGGAGGGGGCAUUUUAAU |
| | 28_6-6_internal_ | AUAUCAAACCCCUAAUCAUC |
| | loop-symmetric_ | |
| | AUAGAA-CGGAAG | |
| 916 | -6_6-6_internal_ | GAUAUUGUGACAUAUCUCUGCACUCAU |
| | loop-symmetric_ | CACACAAAAGAUGCAAAUAGCGCCUAG |
| | UUCAGA-GGCUUU | GAUCCACAGGGAGGGGGCAUUUUAAC |
| | 0_1-1_mismatch_ | AUAUCGGCUUUCUAAUCAUC |
| | A-C | |
| | 29_6-6_internal_ | |
| | loop-symmetric_ | |
| | UAGAAG-AGCGCC | |
| 917 | -6_6-6_internal_ | GAUAUUGUGACAUAUCUCUGCACUCAU |
| | loop-symmetric_ | CACACAAAAGAUGCAAAUAAGUAUUA |
| | UUCAGA-AACAUU | GGAUCCACAGGGAGGGGGCAUUUCAA |
| | 3_1-1_mismatch_ | UAUAUCAACAUUCUAAUCAUC |
| | A-C | |
| | 29_6-6_internal_ | |
| | loop-symmetric_ | |
| | UAGAAG-AAGUAU | |
| 918 | -6_6-6_internal_ | GAUAUUGUGACAUAUCUCUGCACUCAU |
| | loop-symmetric_ | CACACAAAAGAUGCAAAUAGAAUUUA |
| | UUCAGA-AGCUUU | GGAUCCACAGGGAGGGGGCAUCUUAA |
| | 5_1-1_mismatch_ | UAUAUCAGCUUUCUAAUCAUC |
| | A-C | |
| | 29_6-6_internal_ | |
| | loop-symmetric_ | |
| | UAGAAG-AGAAUU | |
| 919 | -6_6-6_internal_ | GAUAUUGUGACAUAUCUCUGCACUCAU |
| | loop-symmetric_ | CACACAAAAGAUGCAAAUAGUAGUUA |
| | UUCAGA-AGAUCU | GGAUCCACAGGGAGGGGGCAUUUUAA |
| | 29_6-6_internal_ | UAUAUCAGAUCUCUAAUCAUC |
| | loop-symmetric_ | |
| | UAGAAG-AGUAGU | |
| 920 | -6_6-6_internal_ | GAUAUUGUGACAUAUCUCUGCACUCAU |
| | loop-symmetric_ | CACACAAAAGAUGCAAAGGCCACAUAG |
| | UUCAGA-AAGUCC | GAUCCACAGGGAGGGGGCAUUUCAAU |
| | 3_1-1_mismatch_ | AUAUCAAGUCCCUAAUCAUC |
| | A-C | |
| | 30_6-6_internal_ | |
| | loop-symmetric_ | |
| | AGAAGA-GGCCAC | |
| 921 | -6_6-6_internal_ | GAUAUUGUGACAUAUCUCUGCACUCAU |
| | loop-symmetric_ | CACACAAAAGAUGCAAAAUAGUGAUA |
| | UUCAGA-CACCGC | GGAUCCACAGGGAGGGGGCAUUCUAA |
| | 4_1-1_mismatch_ | UAUAUCCACCGCCUAAUCAUC |
| | A-C | |
| | 30_6-6_internal_ | |
| | loop-symmetric_ | |
| | AGAAGA-AUAGUG | |
| 922 | -6_6-6_internal_ | GAUAUUGUGACAUAUCUCUGCACUCAU |
| | loop-symmetric_ | CACACAAAAGAUGCAAAGAGGACAUA |
| | UUCAGA-GGUCAU | GGAUCCACAGGGAGGGGGCAUCUUAA |
| | 5_1-1_mismatch_ | UAUAUCGGUCAUCUAAUCAUC |
| | A-C | |
| | 30_6-6_internal_ | |
| | loop-symmetric_ | |
| | AGAAGA-GAGGAC | |
| 923 | -6_6-6_internal_ | GAUAUUGUGACAUAUCUCUGCACUCAU |
| | loop-symmetric_ | CACACAAAAGAUGCAAAGGGCAAAUA |
| | UUCAGA-AGGCAU | GGAUCCACAGGGAGGGGGCAUUUUAA |
| | 30_6-6_internal_ | UAUAUCAGGCAUCUAAUCAUC |
| | loop-symmetric_ | |
| | AGAAGA-GGGCAA | |
| 924 | -6_6-6_internal_ | GAUAUUGUGACAUAUCUCUGCACUCAU |
| | loop-symmetric_ | CACACAAAAGAUGCAACGACCAUAUAG |
| | UUCAGA-CAAAUC | GAUCCACAGGGAGGGGGCAUUUUAAC |
| | 0_1-1_mismatch_ | AUAUCCAAAUCCUAAUCAUC |
| | A-C | |
| | 31_6-6_internal_ | |
| | loop-symmetric_ | |
| | GAAGAU-CGACCA | |
| 925 | -6_6-6_internal_ | GAUAUUGUGACAUAUCUCUGCACUCAU |
| | loop-symmetric_ | CACACAAAAGAUGCAACCAUAAUAUAG |
| | UUCAGA-AGUCGU | GAUCCACAGGGAGGGGGCAUUUCAAU |
| | 3_1-1_mismatch_ | AUAUCAGUCGUCUAAUCAUC |
| | A-C | |
| | 31_6-6_internal_ | |
| | loop-symmetric_ | |
| | GAAGAU-CCAUAA | |
| 926 | -6_6-6_internal_ | GAUAUUGUGACAUAUCUCUGCACUCAU |
| | loop-symmetric_ | CACACAAAAGAUGCAACGAAUAUAUA |
| | UUCAGA-GAUACC | GGAUCCACAGGGAGGGGGCAUUCUAA |
| | 4_1-1_mismatch_ | UAUAUCGAUACCCUAAUCAUC |
| | A-C | |
| | 31_6-6_internal_ | |
| | loop-symmetric_ | |
| | GAAGAU-CGAAUA | |
| 927 | -6_6-6_internal_ | GAUAUUGUGACAUAUCUCUGCACUCAU |
| | loop-symmetric_ | CACACAAAAGAUGCAAUUAGCAUAUA |
| | UUCAGA-GCCAUU | GGAUCCACAGGGAGGGGGCAUCUUAA |
| | 5_1-1_mismatch_ | UAUAUCGCCAUUCUAAUCAUC |
| | A-C | |
| | 31_6-6_internal_ | |
| | loop-symmetric_ | |
| | GAAGAU-UUAGCA | |
| 928 | -6_6-6_internal_ | GAUAUUGUGACAUAUCUCUGCACUCAU |
| | loop-symmetric_ | CACACAAAAGAUGCAACGACGGUAUAG |
| | UUCAGA-GUGUUU | GAUCCACAGGGAGGGGGCAUUUUAAU |
| | 31_6-6_internal_ | AUAUCGUGUUUCUAAUCAUC |
| | loop-symmetric_ | |
| | GAAGAU-CGACGG | |
| 929 | -6_6-6_internal_ | GAUAUUGUGACAUAUCUCUGCACUCAU |
| | loop-symmetric_ | CACACAAAAGAUGCACCUAGACUAUAG |
| | UUCAGA-GGAAUU | GAUCCACAGGGAGGGGGCAUUUUAAC |
| | 0_1-1_mismatch_ | AUAUCGGAAUUCUAAUCAUC |
| | A-C | |
| | 32_6-6_internal_ | |
| | loop-symmetric_ | |
| | AAGAUU-CCUAGA | |
| 930 | -6_6-6_internal_ | GAUAUUGUGACAUAUCUCUGCACUCAU |
| | loop-symmetric_ | CACACAAAAGAUGCAUUGGCCCUAUAG |
| | UUCAGA-CAACAC | GAUCCACAGGGAGGGGGCAUUUCAAU |
| | 3_1-1_mismatch_ | AUAUCCAACACCUAAUCAUC |
| | A-C | |
| | 32_6-6_internal_ | |
| | loop-symmetric_ | |
| | AAGAUU-UUGGCC | |
| 931 | -6_6-6_internal_ | GAUAUUGUGACAUAUCUCUGCACUCAU |
| | loop-symmetric_ | CACACAAAAGAUGCACGGACACUAUAG |
| | UUCAGA-AGAUUU | GAUCCACAGGGAGGGGGCAUUCUAAU |
| | 4_1-1_mismatch_ | AUAUCAGAUUUCUAAUCAUC |
| | A-C | |
| | 32_6-6_internal_ | |
| | loop-symmetric_ | |
| | AAGAUU-CGGACA | |
| 932 | -6_6-6_internal_ | GAUAUUGUGACAUAUCUCUGCACUCAU |
| | loop-symmetric_ | CACACAAAAGAUGCAUCCAUACUAUAG |
| | UUCAGA-CAACUU | GAUCCACAGGGAGGGGGCAUCUUAAU |
| | 5_1-1_mismatch_ | AUAUCCAACUUCUAAUCAUC |
| | A-C | |
| | 32_6-6_internal_ | |
| | loop-symmetric_ | |
| | AAGAUU-UCCAUA | |
| 933 | -6_6-6_internal_ | GAUAUUGUGACAUAUCUCUGCACUCAU |
| | loop-symmetric_ | CACACAAAAGAUGCAUUCGGGCUAUAG |
| | UUCAGA-AGGUUC | GAUCCACAGGGAGGGGGCAUUUUAAU |
| | 32_6-6_internal_ | AUAUCAGGUUCCUAAUCAUC |
| | loop-symmetric_ | |
| | AAGAUU-UUCGGG | |
| 934 | -6_6-6_internal_ | GAUAUUGUGACAUAUCUCUGCACUCAU |
| | loop-symmetric_ | CACACAAAAGAUGCUCCCUAUCUAUAG |
| | UUCAGA-CACCUC | GAUCCACAGGGAGGGGGCAUUUUAAC |
| | 0_1-1_mismatch_ | AUAUCCACCUCCUAAUCAUC |
| | A-C | |
| | 33_6-6_internal_ | |
| | loop-symmetric_ | |
| | AGAUUU-UCCCUA | |
| 935 | -6_6-6_internal_ | GAUAUUGUGACAUAUCUCUGCACUCAU |
| | loop-symmetric_ | CACACAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| 936 | -6_6-6_internal_ | GAUAUUGUGACAUAUCUCUGCACUCAU |
| | loop-symmetric_ | CACACAAAAGAUGCUCGCUGUCUAUAG |
| | UUCAGA-GUACUC | GAUCCACAGGGAGGGGGCAUUCUAAU |
| | 4_1-1_mismatch_ | AUAUCGUACUCCUAAUCAUC |
| | A-C | |
| | 33_6-6_internal_ | |
| | loop-symmetric_ | |
| | AGAUUU-UCGCUG | |
| 937 | -6_6-6_internal_ | GAUAUUGUGACAUAUCUCUGCACUCAU |
| | loop-symmetric_ | CACACAAAAGAUGCUUGCACUCUAUAG |
| | UUCAGA-GGCUUU | GAUCCACAGGGAGGGGGCAUCUUAAU |
| | 5_1-1_mismatch_ | AUAUCGGCUUUCUAAUCAUC |
| | A-C | |
| | 33_6-6_internal_ | |
| | loop-symmetric_ | |
| | AGAUUU-UUGCAC | |
| 938 | -6_6-6_internal_ | GAUAUUGUGACAUAUCUCUGCACUCAU |
| | loop-symmetric_ | CACACAAAAGAUGCCCAGAAUCUAUAG |
| | UUCAGA-AGUAAU | GAUCCACAGGGAGGGGGCAUUUUAAU |
| | 33_6-6_internal_ | AUAUCAGUAAUCUAAUCAUC |
| | loop-symmetric_ | |
| | AGAUUU-CCAGAA | |
| 939 | -6_6-6_internal_ | GAUAUUGUGACAUAUCUCUGCACUCAU |
| | loop-symmetric_ | CACACAAAAGAUGAAUUUAUUCUAUA |
| | UUCAGA-GAUACU | GGAUCCACAGGGAGGGGGCAUUUUAA |
| | 0_1-1_mismatch_ | CAUAUCGAUACUCUAAUCAUC |
| | A-C | |
| | 34_6-6_internal_ | |
| | loop-symmetric_ | |
| | GAUUUG-AAUUUA | |
| 940 | -6_6-6_internal_ | GAUAUUGUGACAUAUCUCUGCACUCAU |
| | loop-symmetric_ | CACACAAAAGAUGGUUGCGUUCUAUA |
| | UUCAGA-ACAUAC | GGAUCCACAGGGAGGGGGCAUUUCAA |
| | 3_1-1_mismatch_ | UAUAUCACAUACCUAAUCAUC |
| | A-C | |
| | 34_6-6_internal_ | |
| | loop-symmetric_ | |
| | GAUUUG-GUUGCG | |
| 941 | -6_6-6_internal_ | GAUAUUGUGACAUAUCUCUGCACUCAU |
| | loop-symmetric_ | CACACAAAAGAUGACCGCAUUCUAUAG |
| | UUCAGA-AGGUGC | GAUCCACAGGGAGGGGGCAUCUUAAU |
| | 5_1-1_mismatch_ | AUAUCAGGUGCCUAAUCAUC |
| | A-C | |
| | 34_6-6_internal_ | |
| | loop-symmetric_ | |
| | GAUUUG-ACCGCA | |
| 942 | -6_6-6_internal_ | GAUAUUGUGACAUAUCUCUGCACUCAU |
| | loop-symmetric_ | CACACAAAAGAUGAACGCAUUCUAUAG |
| | UUCAGA-GCCCCC | GAUCCACAGGGAGGGGGCAUUUUAAU |
| | 34_6-6_internal_ | AUAUCGCCCCCCUAAUCAUC |
| | loop-symmetric_ | |
| | GAUUUG-AACGCA | |
| 943 | -6_6-6_internal_ | GAUAUUGUGACAUAUCUCUGCACUCAU |
| | loop-symmetric_ | CACACAAAAGAUACCACCCUUCUAUAG |
| | UUCAGA-AUAAGC | GAUCCACAGGGAGGGGGCAUUUUAAC |
| | 0_1-1_mismatch_ | AUAUCAUAAGCCUAAUCAUC |
| | A-C | |
| | 35_6-6_internal_ | |
| | loop-symmetric_ | |
| | AUUUGC-ACCACC | |
| 944 | -6_6-6_internal_ | GAUAUUGUGACAUAUCUCUGCACUCAU |
| | loop-symmetric_ | CACACAAAAGAUUAUGCCCUUCUAUAG |
| | UUCAGA-GAAUUC | GAUCCACAGGGAGGGGGCAUUUCAAU |
| | 3_1-1_mismatch_ | AUAUCGAAUUCCUAAUCAUC |
| | A-C | |
| | 35_6-6_internal_ | |
| | loop-symmetric_ | |
| | AUUUGC-UAUGCC | |
| 945 | -6_6-6_internal_ | GAUAUUGUGACAUAUCUCUGCACUCAU |
| | loop-symmetric_ | CACACAAAAGAUUGACCACUUCUAUAG |
| | UUCAGA-AGCAUC | GAUCCACAGGGAGGGGGCAUUCUAAU |
| | 4_1-1_mismatch_ | AUAUCAGCAUCCUAAUCAUC |
| | A-C | |
| | 35_6-6_internal_ | |
| | loop-symmetric_ | |
| | AUUUGC-UGACCA | |
| 946 | -6_6-6_internal_ | GAUAUUGUGACAUAUCUCUGCACUCAU |
| | loop-symmetric_ | CACACAAAAGAUUAGUGCCUUCUAUAG |
| | UUCAGA-AGGUCC | GAUCCACAGGGAGGGGGCAUCUUAAU |
| | 5_1-1_mismatch_ | AUAUCAGGUCCCUAAUCAUC |
| | A-C | |
| | 35_6-6_internal_ | |
| | loop-symmetric_ | |
| | AUUUGC-UAGUGC | |
| 947 | -6_6-6_internal_ | GAUAUUGUGACAUAUCUCUGCACUCAU |
| | loop-symmetric_ | CACACAAAAGAUUUUAUACUUCUAUA |
| | UUCAGA-GGGUUC | GGAUCCACAGGGAGGGGGCAUUUUAA |
| | 35_6-6_internal_ | UAUAUCGGGUUCCUAAUCAUC |
| | loop-symmetric_ | |
| | AUUUGC-UUUAUA | |
| 948 | -6_6-6_internal_ | GAUAUUGUGACAUAUCUCUGCACUCAU |
| | loop-symmetric_ | CACACAAAAGACUUACCUCUUCUAUAG |
| | UUCAGA-GGAUAU | GAUCCACAGGGAGGGGGCAUUUUAAC |
| | 0_1-1_mismatch_ | AUAUCGGAUAUCUAAUCAUC |
| | A-C | |
| | 36_6-6_internal_ | |
| | loop-symmetric_ | |
| | UUUGCA-CUUACC | |
| 949 | -6_6-6_internal_ | GAUAUUGUGACAUAUCUCUGCACUCAU |
| | loop-symmetric_ | CACACAAAAGACUAGUUUCUUCUAUAG |
| | UUCAGA-AGCUAU | GAUCCACAGGGAGGGGGCAUUUCAAU |
| | 3_1-1_mismatch_ | AUAUCAGCUAUCUAAUCAUC |
| | A-C | |
| | 36_6-6_internal_ | |
| | loop-symmetric_ | |
| | UUUGCA-CUAGUU | |
| 950 | -6_6-6_internal_ | GAUAUUGUGACAUAUCUCUGCACUCAU |
| | loop-symmetric_ | CACACAAAAGACAACCUUCUUCUAUAG |
| | UUCAGA-GAGACC | GAUCCACAGGGAGGGGGCAUUCUAAU |
| | 4_1-1_mismatch_ | AUAUCGAGACCCUAAUCAUC |
| | A-C | |
| | 36_6-6_internal_ | |
| | loop-symmetric_ | |
| | UUUGCA-CAACCU | |
| 951 | -6_6-6_internal_ | GAUAUUGUGACAUAUCUCUGCACUCAU |
| | loop-symmetric_ | CACACAAAAGAAAUCGCUCUUCUAUAG |
| | UUCAGA-CACUCU | GAUCCACAGGGAGGGGGCAUCUUAAU |
| | 5_1-1_mismatch_ | AUAUCCACUCUCUAAUCAUC |
| | A-C | |
| | 36_6-6_internal_ | |
| | loop-symmetric_ | |
| | UUUGCA-AAUCGC | |
| 952 | -6_6-6_internal_ | GAUAUUGUGACAUAUCUCUGCACUCAU |
| | loop-symmetric_ | CACACAAAAGAACAUUUUCUUCUAUAG |
| | UUCAGA-AGCACU | GAUCCACAGGGAGGGGGCAUUUUAAU |
| | 36_6-6_internal_ | AUAUCAGCACUCUAAUCAUC |
| | loop-symmetric_ | |
| | UUUGCA-ACAUUU | |
| 953 | -6_6-6_internal_ | GAUAUUGUGACAUAUCUCUGCACUCAU |
| | loop-symmetric_ | CACACAAAAGCCCUAUAUCUUCUAUAG |
| | UUCAGA-GGUUUC | GAUCCACAGGGAGGGGGCAUUUUAAC |
| | 0_1-1_mismatch_ | AUAUCGGUUUCCUAAUCAUC |
| | A-C | |
| | 37_6-6_internal_ | |
| | loop-symmetric_ | |
| | UUGCAU-CCCUAU | |
| 954 | -6_6-6_internal_ | GAUAUUGUGACAUAUCUCUGCACUCAU |
| | loop-symmetric_ | CACACAAAAGUACUCUAUCUUCUAUAG |
| | UUCAGA-GAGCCU | GAUCCACAGGGAGGGGGCAUUUCAAU |
| | 3_1-1_mismatch_ | AUAUCGAGCCUCUAAUCAUC |
| | A-C | |
| | 37_6-6_internal_ | |
| | loop-symmetric_ | |
| | UUGCAU-UACUCU | |
| 955 | -6_6-6_internal_ | GAUAUUGUGACAUAUCUCUGCACUCAU |
| | loop-symmetric_ | CACACAAAAGCACGUUAUCUUCUAUAG |
| | UUCAGA-GUGCCC | GAUCCACAGGGAGGGGGCAUUCUAAU |
| | 4_1-1_mismatch_ | AUAUCGUGCCCCUAAUCAUC |
| | A-C | |
| | 37_6-6_internal_ | |
| | loop-symmetric_ | |
| | UUGCAU-CACGUU | |
| 956 | -6_6-6_internal_ | GAUAUUGUGACAUAUCUCUGCACUCAU |
| | loop-symmetric_ | CACACAAAAGCACACUAUCUUCUAUAG |
| | UUCAGA-GGGCCC | GAUCCACAGGGAGGGGGCAUCUUAAU |
| | 5_1-1_mismatch_ | AUAUCGGGCCCCUAAUCAUC |
| | A-C | |
| | 37_6-6_internal_ | |
| | loop-symmetric_ | |
| | UUGCAU-CACACU | |
| 957 | -6_6-6_internal_ | GAUAUUGUGACAUAUCUCUGCACUCAU |
| | loop-symmetric_ | CACACAAAAGCACUCCAUCUUCUAUAG |
| | UUCAGA-AGGUUC | GAUCCACAGGGAGGGGGCAUUUUAAU |
| | 37_6-6_internal_ | AUAUCAGGUUCCUAAUCAUC |
| | loop-symmetric_ | |
| | UUGCAU-CACUCC | |
| 958 | -6_6-6_internal_ | GAUAUUGUGACAUAUCUCUGCACUCAU |
| | loop-symmetric_ | CACACAAAACUGAACAAUCUUCUAUAG |
| | UUCAGA-GACAGC | GAUCCACAGGGAGGGGGCAUUUCAAU |
| | 3_1-1_mismatch_ | AUAUCGACAGCCUAAUCAUC |
| | A-C | |
| | 38_6-6_internal_ | |
| | loop-symmetric_ | |
| | UGCAUC-CUGAAC | |
| 959 | -6_6-6_internal_ | GAUAUUGUGACAUAUCUCUGCACUCAU |
| | loop-symmetric_ | CACACAAAAUCGUCUAAUCUUCUAUAG |
| | UUCAGA-GGUUAU | GAUCCACAGGGAGGGGGCAUUCUAAU |
| | 4_1-1_mismatch_ | AUAUCGGUUAUCUAAUCAUC |
| | A-C | |
| | 38_6-6_internal_ | |
| | loop-symmetric_ | |
| | UGCAUC-UCGUCU | |
| 960 | -6_6-6_internal_ | GAUAUUGUGACAUAUCUCUGCACUCAU |
| | loop-symmetric_ | CACACAAAAUCGACCAAUCUUCUAUAG |
| | UUCAGA-GGAAUC | GAUCCACAGGGAGGGGGCAUCUUAAU |
| | 5_1-1_mismatch_ | AUAUCGGAAUCCUAAUCAUC |
| | A-C | |
| | 38_6-6_internal_ | |
| | loop-symmetric_ | |
| | UGCAUC-UCGACC | |
| 961 | -6_6-6_internal_ | GAUAUUGUGACAUAUCUCUGCACUCAU |
| | loop-symmetric_ | CACACAAAACCCAGUAAUCUUCUAUAG |
| | UUCAGA-GCAUCC | GAUCCACAGGGAGGGGGCAUUUUAAU |
| | 38_6-6_internal_ | AUAUCGCAUCCCUAAUCAUC |
| | loop-symmetric_ | |
| | UGCAUC-CCCAGU | |
| 962 | -6_6-6_internal_ | GAUAUUGUGACAUAUCUCUGCACUCAU |
| | loop-symmetric_ | CACACAAAUCGCAGAAAUCUUCUAUAG |
| | UUCAGA-GGGCAC | GAUCCACAGGGAGGGGGCAUUUUAAC |
| | 0_1-1_mismatch_ | AUAUCGGGCACCUAAUCAUC |
| | A-C | |
| | 39_6-6_internal_ | |
| | loop-symmetric_ | |
| | GCAUCU-UCGCAG | |
| 963 | -6_6-6_internal_ | GAUAUUGUGACAUAUCUCUGCACUCAU |
| | loop-symmetric_ | CACACAAAUUUUUAAAAUCUUCUAUA |
| | UUCAGA-GGAAGC | GGAUCCACAGGGAGGGGGCAUUUCAA |
| | 3_1-1_mismatch_ | UAUAUCGGAAGCCUAAUCAUC |
| | A-C | |
| | 39_6-6_internal_ | |
| | loop-symmetric_ | |
| | GCAUCU-UUUUUA | |
| 964 | -6_6-6_internal_ | GAUAUUGUGACAUAUCUCUGCACUCAU |
| | loop-symmetric_ | CACACAAAUAAAUAAAAUCUUCUAUA |
| | UUCAGA-AGAUUC | GGAUCCACAGGGAGGGGGCAUUCUAA |
| | 4_1-1_mismatch_ | UAUAUCAGAUUCCUAAUCAUC |
| | A-C | |
| | 39_6-6_internal_ | |
| | loop-symmetric_ | |
| | GCAUCU-UAAAUA | |
| 965 | -6_6-6_internal_ | GAUAUUGUGACAUAUCUCUGCACUCAU |
| | loop-symmetric_ | CACACAAAUAUUUAAAAUCUUCUAUA |
| | UUCAGA-AUACGU | GGAUCCACAGGGAGGGGGCAUCUUAA |
| | 5_1-1_mismatch_ | UAUAUCAUACGUCUAAUCAUC |
| | A-C | |
| | 39_6-6_internal_ | |
| | loop-symmetric_ | |
| | GCAUCU-UAUUUA | |
| 966 | -6_6-6_internal_ | GAUAUUGUGACAUAUCUCUGCACUCAU |
| | loop-symmetric_ | CACACAAACAUCCGAAAUCUUCUAUAG |
| | UUCAGA-CACAGC | GAUCCACAGGGAGGGGGCAUUUUAAU |
| | 39_6-6_internal_ | AUAUCCACAGCCUAAUCAUC |
| | loop-symmetric_ | |
| | GCAUCU-CAUCCG | |
| 967 | -6_6-6_internal_ | GAUAUUGUGACAUAUCUCUGCACUCAU |
| | loop-symmetric_ | CACACAAUGCUCCCAAAUCUUCUAUAG |
| | UUCAGA-AUAAGU | GAUCCACAGGGAGGGGGCAUUUUAAC |
| | 0_1-1_mismatch_ | AUAUCAUAAGUCUAAUCAUC |
| | A-C | |
| | 40_6-6_internal_ | |
| | loop-symmetric_ | |
| | CAUCUU-UGCUCC | |
| 968 | -6_6-6_internal_ | GAUAUUGUGACAUAUCUCUGCACUCAU |
| | loop-symmetric_ | CACACAACUUUUUCAAAUCUUCUAUAG |
| | UUCAGA-AUAUUU | GAUCCACAGGGAGGGGGCAUUUCAAU |
| | 3_1-1_mismatch_ | AUAUCAUAUUUCUAAUCAUC |
| | A-C | |
| | 40_6-6_internal_ | |
| | loop-symmetric_ | |
| | CAUCUU-CUUUUU | |
| 969 | -6_6-6_internal_ | GAUAUUGUGACAUAUCUCUGCACUCAU |
| | loop-symmetric_ | CACACAAUCAACUCAAAUCUUCUAUAG |
| | UUCAGA-GGAAUU | GAUCCACAGGGAGGGGGCAUUCUAAU |
| | 4_1-1_mismatch_ | AUAUCGGAAUUCUAAUCAUC |
| | A-C | |
| | 40_6-6_internal_ | |
| | loop-symmetric_ | |
| | CAUCUU-UCAACU | |
| 970 | -6_6-6_internal_ | GAUAUUGUGACAUAUCUCUGCACUCAU |
| | loop-symmetric_ | CACACAACUACACCAAAUCUUCUAUAG |
| | UUCAGA-AAAAUU | GAUCCACAGGGAGGGGGCAUCUUAAU |
| | 5_1-1_mismatch_ | AUAUCAAAAUUCUAAUCAUC |
| | A-C | |
| | 40_6-6_internal_ | |
| | loop-symmetric_ | |
| | CAUCUU-CUACAC | |
| 971 | -6_6-6_internal_ | GAUAUUGUGACAUAUCUCUGCACUCAU |
| | loop-symmetric_ | CACACACAAAUCGCAAAUCUUCUAUAG |
| | UUCAGA-CAUUUU | GAUCCACAGGGAGGGGGCAUUUUAAC |
| | 0_1-1_mismatch_ | AUAUCCAUUUUCUAAUCAUC |
| | A-C | |
| | 41_6-6_internal_ | |
| | loop-symmetric_ | |
| | AUCUUU-CAAAUC | |
| 972 | -6_6-6_internal_ | GAUAUUGUGACAUAUCUCUGCACUCAU |
| | loop-symmetric_ | CACACACGCAAAGCAAAUCUUCUAUAG |
| | UUCAGA-CACGCC | GAUCCACAGGGAGGGGGCAUUUCAAU |
| | 3_1-1_mismatch_ | AUAUCCACGCCCUAAUCAUC |
| | A-C | |
| | 41_6-6_internal_ | |
| | loop-symmetric_ | |
| | AUCUUU-CGCAAA | |
| 973 | -6_6-6_internal_ | GAUAUUGUGACAUAUCUCUGCACUCAU |
| | loop-symmetric_ | CACACAUCACAAGCAAAUCUUCUAUAG |
| | UUCAGA-GUCAAU | GAUCCACAGGGAGGGGGCAUUCUAAU |
| | 4_1-1_mismatch_ | AUAUCGUCAAUCUAAUCAUC |
| | A-C | |
| | 41_6-6_internal_ | |
| | loop-symmetric_ | |
| | AUCUUU-UCACAA | |
| 974 | -6_6-6_internal_ | GAUAUUGUGACAUAUCUCUGCACUCAU |
| | loop-symmetric_ | CACACACCGCUGGCAAAUCUUCUAUAG |
| | UUCAGA-GUAUUC | GAUCCACAGGGAGGGGGCAUCUUAAU |
| | 5_1-1_mismatch_ | AUAUCGUAUUCCUAAUCAUC |
| | A-C | |
| | 41_6-6_internal_ | |
| | loop-symmetric_ | |
| | AUCUUU-CCGCUG | |
| 975 | -6_6-6_internal_ | GAUAUUGUGACAUAUCUCUGCACUCAU |
| | loop-symmetric_ | CACACACACUCAGCAAAUCUUCUAUAG |
| | UUCAGA-GAUCUU | GAUCCACAGGGAGGGGGCAUUUUAAU |
| | 41_6-6_internal_ | AUAUCGAUCUUCUAAUCAUC |
| | loop-symmetric_ | |
| | AUCUUU-CACUCA | |
| 976 | -6_6-6_internal_ | GAUAUUGUGACAUAUCUCUGCACUCAU |
| | loop-symmetric_ | CACACCGUCGUUGCAAAUCUUCUAUAG |
| | UUCAGA-GUGUUU | GAUCCACAGGGAGGGGGCAUUUUAAC |
| | 0_1-1_mismatch_ | AUAUCGUGUUUCUAAUCAUC |
| | A-C | |
| | 42_6-6_internal_ | |
| | loop-symmetric_ | |
| | UCUUUU-CGUCGU | |
| 977 | -6_6-6_internal_ | GAUAUUGUGACAUAUCUCUGCACUCAU |
| | loop-symmetric_ | CACACCUGUCCUGCAAAUCUUCUAUAG |
| | UUCAGA-AGCAUC | GAUCCACAGGGAGGGGGCAUUUCAAU |
| | 3_1-1_mismatch_ | AUAUCAGCAUCCUAAUCAUC |
| | A-C | |
| | 42_6-6_internal_ | |
| | loop-symmetric_ | |
| | UCUUUU-CUGUCC | |
| 978 | -6_6-6_internal_ | GAUAUUGUGACAUAUCUCUGCACUCAU |
| | loop-symmetric_ | CACACCGCUUCUGCAAAUCUUCUAUAG |
| | UUCAGA-GGGUUC | GAUCCACAGGGAGGGGGCAUUCUAAU |
| | 4_1-1_mismatch_ | AUAUCGGGUUCCUAAUCAUC |
| | A-C | |
| | 42_6-6_internal_ | |
| | loop-symmetric_ | |
| | UCUUUU-CGCUUC | |
| 979 | -6_6-6_internal_ | GAUAUUGUGACAUAUCUCUGCACUCAU |
| | loop-symmetric_ | CACACUAACACUGCAAAUCUUCUAUAG |
| | UUCAGA-AACUCC | GAUCCACAGGGAGGGGGCAUCUUAAU |
| | 5_1-1_mismatch_ | AUAUCAACUCCCUAAUCAUC |
| | A-C | |
| | 42_6-6_internal_ | |
| | loop-symmetric_ | |
| | UCUUUU-UAACAC | |
| 980 | -6_6-6_internal_ | GAUAUUGUGACAUAUCUCUGCACUCAU |
| | loop-symmetric_ | CACACUUUACUUGCAAAUCUUCUAUAG |
| | UUCAGA-GUGCUU | GAUCCACAGGGAGGGGGCAUUUUAAU |
| | 42_6-6_internal_ | AUAUCGUGCUUCUAAUCAUC |
| | loop-symmetric_ | |
| | UCUUUU-UUUACU | |
| 981 | -6_6-6_internal_ | GAUAUUGUGACAUAUCUCUGCACUCAU |
| | loop-symmetric_ | CACAGUCAUCAUGCAAAUCUUCUAUAG |
| | UUCAGA-AAAAAC | GAUCCACAGGGAGGGGGCAUUUCAAU |
| | 3_1-1_mismatch_ | AUAUCAAAAACCUAAUCAUC |
| | A-C | |
| | 43_6-6_internal_ | |
| | loop-symmetric_ | |
| | CUUUUG-GUCAUC | |
| 982 | -6_6-6_internal_ | GAUAUUGUGACAUAUCUCUGCACUCAU |
| | loop-symmetric_ | CACAAUGUGAAUGCAAAUCUUCUAUA |
| | UUCAGA-GUAUCU | GGAUCCACAGGGAGGGGGCAUUCUAA |
| | 4_1-1_mismatch_ | UAUAUCGUAUCUCUAAUCAUC |
| | A-C | |
| | 43_6-6_internal_ | |
| | loop-symmetric_ | |
| | CUUUUG-AUGUGA | |
| 983 | -6_6-6_internal_ | GAUAUUGUGACAUAUCUCUGCACUCAU |
| | loop-symmetric_ | CACAAUUACUAUGCAAAUCUUCUAUAG |
| | UUCAGA-AAAACU | GAUCCACAGGGAGGGGGCAUCUUAAU |
| | 5_1-1_mismatch_ | AUAUCAAAACUCUAAUCAUC |
| | A-C | |
| | 43_6-6_internal_ | |
| | loop-symmetric_ | |
| | CUUUUG-AUUACU | |
| 984 | -6_6-6_internal_ | GAUAUUGUGACAUAUCUCUGCACUCAU |
| | loop-symmetric_ | CACAGCUUAAAUGCAAAUCUUCUAUAG |
| | UUCAGA-AACACC | GAUCCACAGGGAGGGGGCAUUUUAAU |
| | 43_6-6_internal_ | AUAUCAACACCCUAAUCAUC |
| | loop-symmetric_ | |
| | CUUUUG-GCUUAA | |
| 985 | -6_6-6_internal_ | GAUAUUGUGACAUAUCUCUGCACUCAU |
| | loop-symmetric_ | CACUUGGUCGAUGCAAAUCUUCUAUAG |
| | UUCAGA-GGGCCU | GAUCCACAGGGAGGGGGCAUUUUAAC |
| | 0_1-1_mismatch_ | AUAUCGGGCCUCUAAUCAUC |
| | A-C | |
| | 44_6-6_internal_ | |
| | loop-symmetric_ | |
| | UUUUGU-UUGGUC | |
| 986 | -6_6-6_internal_ | GAUAUUGUGACAUAUCUCUGCACUCAU |
| | loop-symmetric_ | CACUGAUCCGAUGCAAAUCUUCUAUAG |
| | UUCAGA-AACAUC | GAUCCACAGGGAGGGGGCAUUUCAAU |
| | 3_1-1_mismatch_ | AUAUCAACAUCCUAAUCAUC |
| | A-C | |
| | 44_6-6_internal_ | |
| | loop-symmetric_ | |
| | UUUUGU-UGAUCC | |
| 987 | -6_6-6_internal_ | GAUAUUGUGACAUAUCUCUGCACUCAU |
| | loop-symmetric_ | CACUUGCUCGAUGCAAAUCUUCUAUAG |
| | UUCAGA-CACACU | GAUCCACAGGGAGGGGGCAUUCUAAU |
| | 4_1-1_mismatch_ | AUAUCCACACUCUAAUCAUC |
| | A-C | |
| | 44_6-6_internal_ | |
| | loop-symmetric_ | |
| | UUUUGU-UUGCUC | |
| 988 | -6_6-6_internal_ | GAUAUUGUGACAUAUCUCUGCACUCAU |
| | loop-symmetric_ | CACUGUCCUGAUGCAAAUCUUCUAUAG |
| | UUCAGA-GUAAGC | GAUCCACAGGGAGGGGGCAUCUUAAU |
| | 5_1-1_mismatch_ | AUAUCGUAAGCCUAAUCAUC |
| | A-C | |
| | 44_6-6_internal_ | |
| | loop-symmetric_ | |
| | UUUUGU-UGUCCU | |
| 989 | -6_6-6_internal_ | GAUAUUGUGACAUAUCUCUGCACUCAU |
| | loop-symmetric_ | CACUGUAUCGAUGCAAAUCUUCUAUAG |
| | UUCAGA-CAAUUU | GAUCCACAGGGAGGGGGCAUUUUAAU |
| | 44_6-6_internal_ | AUAUCCAAUUUCUAAUCAUC |
| | loop-symmetric_ | |
| | UUUUGU-UGUAUC | |
| 990 | -5_6-6_internal_ | GAUAUUGUGACAUAUCUCUGCACUCAU |
| | loop-symmetric_ | CACACAAAAGAUGCAAAUCUUCUAUAG |
| | UCAGAG-GAAGCU | GAUCCACAGGGAGGGGGCAUUUUAAC |
| | 0_1-1_mismatch_ | AUAUGAAGCUACUAAUCAUC |
| | A-C | |
| 991 | -5_6-6_internal_ | GAUAUUGUGACAUAUCUCUGCACUCAU |
| | loop-symmetric_ | CACACAAAAGAUGCAAAUCUUCUAUAG |
| | UCAGAG-GCAAAU | GAUCCACAGGGAGGGGGCAUUUCAAU |
| | 3_1-1_mismatch_ | AUAUGCAAAUACUAAUCAUC |
| | A-C | |
| 992 | -5_6-6_internal_ | GAUAUUGUGACAUAUCUCUGCACUCAU |
| | loop-symmetric_ | CACACAAAAGAUGCAAAUCUUCUAUAG |
| | UCAGAG-AGGGCU | GAUCCACAGGGAGGGGGCAUUCUAAU |
| | 4_1-1_mismatch_ | AUAUAGGGCUACUAAUCAUC |
| | A-C | |
| 993 | -5_6-6_internal_ | GAUAUUGUGACAUAUCUCUGCACUCAU |
| | loop-symmetric_ | CACACAAAAGAUGCAAAUCUUCUAUAG |
| | UCAGAG-GCAUAC | GAUCCACAGGGAGGGGGCAUCUUAAU |
| | 5_1-1_mismatch_ | AUAUGCAUACACUAAUCAUC |
| | A-C | |
| 994 | -5_6-6_internal_ | GAUAUUGUGACAUAUCUCUGCACUCAU |
| | loop-symmetric_ | CACACAAAAGAUGCAAAUCUUCUAUAG |
| | UCAGAG-AGAGAU | GAUCCACAGGGAGGGGGCAUUUUAAU |
| | | AUAUAGAGAUACUAAUCAUC |
| 995 | -5_6-6_internal_ | GAUAUUGUGACAUAUCUCUGCACUCAU |
| | loop-symmetric_ | CACACAAAAGAUGCAAAUCUUCUAUAU |
| | UCAGAG-AGGCGU | CGCGAACAGGGAGGGGGCAUUUUAAC |
| | 0_1-1_mismatch_ | AUAUAGGCGUACUAAUCAUC |
| | A-C | |
| | 21_6-6_internal_ | |
| | loop-symmetric_ | |
| | GGAUCC-UCGCGA | |
| 996 | -5_6-6_internal_ | GAUAUUGUGACAUAUCUCUGCACUCAU |
| | loop-symmetric_ | CACACAAAAGAUGCAAAUCUUCUAUAA |
| | UCAGAG-GAGAAU | CCCUAACAGGGAGGGGGCAUUUCAAUA |
| | 3_1-1_mismatch_ | UAUGAGAAUACUAAUCAUC |
| | A-C | |
| | 21_6-6_internal_ | |
| | loop-symmetric_ | |
| | GGAUCC-ACCCUA | |
| 997 | -5_6-6_internal_ | GAUAUUGUGACAUAUCUCUGCACUCAU |
| | loop-symmetric_ | CACACAAAAGAUGCAAAUCUUCUAUAU |
| | UCAGAG-AACCAC | CAGUAACAGGGAGGGGGCAUCUUAAU |
| | 5_1-1_mismatch_ | AUAUAACCACACUAAUCAUC |
| | A-C | |
| | 21_6-6_internal_ | |
| | loop-symmetric_ | |
| | GGAUCC-UCAGUA | |
| 998 | -5_6-6_internal_ | GAUAUUGUGACAUAUCUCUGCACUCAU |
| | loop-symmetric_ | CACACAAAAGAUGCAAAUCUUCUAUAC |
| | UCAGAG-AGGUAU | UAGUAACAGGGAGGGGGCAUUUUAAU |
| | 21_6-6_internal_ | AUAUAGGUAUACUAAUCAUC |
| | loop-symmetric_ | |
| | GGAUCC-CUAGUA | |
| 999 | -5_6-6_internal_ | GAUAUUGUGACAUAUCUCUGCACUCAU |
| | loop-symmetric_ | CACACAAAAGAUGCAAAUCUUCUAUCA |
| | UCAGAG-GGAACC | CUGGCACAGGGAGGGGGCAUUUUAAC |
| | 0_1-1_mismatch_ | AUAUGGAACCACUAAUCAUC |
| | A-C | |
| | 22_6-6_internal_ | |
| | loop-symmetric_ | |
| | GAUCCU-CACUGG | |
| 1000 | -5_6-6_internal_ | GAUAUUGUGACAUAUCUCUGCACUCAU |
| | loop-symmetric_ | CACACAAAAGAUGCAAAUCUUCUAUUA |
| | UCAGAG-AUGCAC | ACAGCACAGGGAGGGGGCAUUUCAAU |
| | 3_1-1_mismatch_ | AUAUAUGCACACUAAUCAUC |
| | A-C | |
| | 22_6-6_internal_ | |
| | loop-symmetric_ | |
| | GAUCCU-UAACAG | |
| 1001 | -5_6-6_internal_ | GAUAUUGUGACAUAUCUCUGCACUCAU |
| | loop-symmetric_ | CACACAAAAGAUGCAAAUCUUCUAUUU |
| | UCAGAG-GGAAAU | AACACACAGGGAGGGGGCAUUCUAAU |
| | 4_1-1_mismatch_ | AUAUGGAAAUACUAAUCAUC |
| | A-C | |
| | 22_6-6_internal_ | |
| | loop-symmetric_ | |
| | GAUCCU-UUAACA | |
| 1002 | -5_6-6_internal_ | GAUAUUGUGACAUAUCUCUGCACUCAU |
| | loop-symmetric_ | CACACAAAAGAUGCAAAUCUUCUAUCC |
| | UCAGAG-AAGGCU | CUAACACAGGGAGGGGGCAUCUUAAU |
| | 5_1-1_mismatch_ | AUAUAAGGCUACUAAUCAUC |
| | A-C | |
| | 22_6-6_internal_ | |
| | loop-symmetric_ | |
| | GAUCCU-CCCUAA | |
| 1003 | -5_6-6_internal_ | GAUAUUGUGACAUAUCUCUGCACUCAU |
| | loop-symmetric_ | CACACAAAAGAUGCAAAUCUUCUAUUA |
| | UCAGAG-GAGAGU | GCAGCACAGGGAGGGGGCAUUUUAAU |
| | 22_6-6_internal_ | AUAUGAGAGUACUAAUCAUC |
| | loop-symmetric_ | |
| | GAUCCU-UAGCAG | |
| 1004 | -5_6-6_internal_ | GAUAUUGUGACAUAUCUCUGCACUCAU |
| | loop-symmetric_ | CACACAAAAGAUGCAAAUCUUCUAACA |
| | UCAGAG-GCAUUU | AUGCCACAGGGAGGGGGCAUUUCAAU |
| | 3_1-1_mismatch_ | AUAUGCAUUUACUAAUCAUC |
| | A-C | |
| | 23_6-6_internal_ | |
| | loop-symmetric_ | |
| | AUCCUA-ACAAUG | |
| 1005 | -5_6-6_internal_ | GAUAUUGUGACAUAUCUCUGCACUCAU |
| | loop-symmetric_ | CACACAAAAGAUGCAAAUCUUCUACUU |
| | UCAGAG-GCAUAC | CGCCCACAGGGAGGGGGCAUUCUAAUA |
| | 4_1-1_mismatch_ | UAUGCAUACACUAAUCAUC |
| | A-C | |
| | 23_6-6_internal_ | |
| | loop-symmetric_ | |
| | AUCCUA-CUUCGC | |
| 1006 | -5_6-6_internal_ | GAUAUUGUGACAUAUCUCUGCACUCAU |
| | loop-symmetric_ | CACACAAAAGAUGCAAAUCUUCUAGUU |
| | UCAGAG-AUAGUC | AAGCCACAGGGAGGGGGCAUCUUAAU |
| | 5_1-1_mismatch_ | AUAUAUAGUCACUAAUCAUC |
| | A-C | |
| | 23_6-6_internal_ | |
| | loop-symmetric_ | |
| | AUCCUA-GUUAAG | |
| 1007 | -5_6-6_internal_ | GAUAUUGUGACAUAUCUCUGCACUCAU |
| | loop-symmetric_ | CACACAAAAGAUGCAAAUCUUCUAAUU |
| | UCAGAG-AGAUAC | AAGCCACAGGGAGGGGGCAUUUUAAU |
| | 23_6-6_internal_ | AUAUAGAUACACUAAUCAUC |
| | loop-symmetric_ | |
| | AUCCUA-AUUAAG | |
| 1008 | -5_6-6_internal_ | GAUAUUGUGACAUAUCUCUGCACUCAU |
| | loop-symmetric_ | CACACAAAAGAUGCAAAUCUUCUUUUU |
| | UCAGAG-GAGAGU | CUUCCACAGGGAGGGGGCAUUUUAACA |
| | 0_1-1_mismatch_ | UAUGAGAGUACUAAUCAUC |
| | A-C | |
| | 24_6-6_internal_ | |
| | loop-symmetric_ | |
| | UCCUAU-UUUUCU | |
| 1009 | -5_6-6_internal_ | GAUAUUGUGACAUAUCUCUGCACUCAU |
| | loop-symmetric_ | CACACAAAAGAUGCAAAUCUUCUUAUU |
| | UCAGAG-AUAACU | GUUCCACAGGGAGGGGGCAUUUCAAU |
| | 3_1-1_mismatch_ | AUAUAUAACUACUAAUCAUC |
| | A-C | |
| | 24_6-6_internal_ | |
| | loop-symmetric_ | |
| | UCCUAU-UAUUGU | |
| 1010 | -5_6-6_internal_ | GAUAUUGUGACAUAUCUCUGCACUCAU |
| | loop-symmetric_ | CACACAAAAGAUGCAAAUCUUCUUGAC |
| | UCAGAG-AAACCC | ACUCCACAGGGAGGGGGCAUUCUAAUA |
| | 4_1-1_mismatch_ | UAUAAACCCACUAAUCAUC |
| | A-C | |
| | 24_6-6_internal_ | |
| | loop-symmetric_ | |
| | UCCUAU-UGACAC | |
| 1011 | -5_6-6_internal_ | GAUAUUGUGACAUAUCUCUGCACUCAU |
| | loop-symmetric_ | CACACAAAAGAUGCAAAUCUUCUCUUA |
| | UCAGAG-GAACCU | UCUCCACAGGGAGGGGGCAUCUUAAUA |
| | 5_1-1_mismatch_ | UAUGAACCUACUAAUCAUC |
| | A-C | |
| | 24_6-6_internal_ | |
| | loop-symmetric_ | |
| | UCCUAU-CUUAUC | |
| 1012 | -5_6-6_internal_ | GAUAUUGUGACAUAUCUCUGCACUCAU |
| | loop-symmetric_ | CACACAAAAGAUGCAAAUCUUCUUGUA |
| | UCAGAG-AACCAC | CCUCCACAGGGAGGGGGCAUUUUAAUA |
| | 24_6-6_internal_ | UAUAACCACACUAAUCAUC |
| | loop-symmetric_ | |
| | UCCUAU-UGUACC | |
| 1013 | -5_6-6_internal_ | GAUAUUGUGACAUAUCUCUGCACUCAU |
| | loop-symmetric_ | CACACAAAAGAUGCAAAUCUUCACUUC |
| | UCAGAG-AUUACC | AAUCCACAGGGAGGGGGCAUUUUAAC |
| | 0_1-1_mismatch_ | AUAUAUUACCACUAAUCAUC |
| | A-C | |
| | 25_6-6_internal_ | |
| | loop-symmetric_ | |
| | CCUAUA-ACUUCA | |
| 1014 | -5_6-6_internal_ | GAUAUUGUGACAUAUCUCUGCACUCAU |
| | loop-symmetric_ | CACACAAAAGAUGCAAAUCUUCCAGUC |
| | UCAGAG-GAUACC | UAUCCACAGGGAGGGGGCAUUUCAAU |
| | 3_1-1_mismatch_ | AUAUGAUACCACUAAUCAUC |
| | A-C | |
| | 25_6-6_internal_ | |
| | loop-symmetric_ | |
| | CCUAUA-CAGUCU | |
| 1015 | -5_6-6_internal_ | GAUAUUGUGACAUAUCUCUGCACUCAU |
| | loop-symmetric_ | CACACAAAAGAUGCAAAUCUUCCCCAC |
| | UCAGAG-GGGACU | CAUCCACAGGGAGGGGGCAUUCUAAUA |
| | 4_1-1_mismatch_ | UAUGGGACUACUAAUCAUC |
| | A-C | |
| | 25_6-6_internal_ | |
| | loop-symmetric_ | |
| | CCUAUA-CCCACC | |
| 1016 | -5_6-6_internal_ | GAUAUUGUGACAUAUCUCUGCACUCAU |
| | loop-symmetric_ | CACACAAAAGAUGCAAAUCUUCGACUC |
| | UCAGAG-GCAUCC | CAUCCACAGGGAGGGGGCAUCUUAAUA |
| | 5_1-1_mismatch_ | UAUGCAUCCACUAAUCAUC |
| | A-C | |
| | 25_6-6_internal_ | |
| | loop-symmetric_ | |
| | CCUAUA-GACUCC | |
| 1017 | -5_6-6_internal_ | GAUAUUGUGACAUAUCUCUGCACUCAU |
| | loop-symmetric_ | CACACAAAAGAUGCAAAUCUUCACCGA |
| | UCAGAG-GCAUAU | AAUCCACAGGGAGGGGGCAUUUUAAU |
| | 25_6-6_internal_ | AUAUGCAUAUACUAAUCAUC |
| | loop-symmetric_ | |
| | CCUAUA-ACCGAA | |
| 1018 | -5_6-6_internal_ | GAUAUUGUGACAUAUCUCUGCACUCAU |
| | loop-symmetric_ | CACACAAAAGAUGCAAAUCUUAGUCUC |
| | UCAGAG-GAACCC | GAUCCACAGGGAGGGGGCAUUUCAAU |
| | 3_1-1_mismatch_ | AUAUGAACCCACUAAUCAUC |
| | A-C | |
| | 26_6-6_internal_ | |
| | loop-symmetric_ | |
| | CUAUAG-AGUCUC | |
| 1019 | -5_6-6_internal_ | GAUAUUGUGACAUAUCUCUGCACUCAU |
| | loop-symmetric_ | CACACAAAAGAUGCAAAUCUUAAUCCU |
| | UCAGAG-AACAAC | GAUCCACAGGGAGGGGGCAUUCUAAU |
| | 4_1-1_mismatch_ | AUAUAACAACACUAAUCAUC |
| | A-C | |
| | 26_6-6_internal_ | |
| | loop-symmetric_ | |
| | CUAUAG-AAUCCU | |
| 1020 | -5_6-6_internal_ | GAUAUUGUGACAUAUCUCUGCACUCAU |
| | loop-symmetric_ | CACACAAAAGAUGCAAAUCUUGGCACC |
| | UCAGAG-AGAGUU | GAUCCACAGGGAGGGGGCAUCUUAAU |
| | 5_1-1_mismatch_ | AUAUAGAGUUACUAAUCAUC |
| | A-C | |
| | 26_6-6_internal_ | |
| | loop-symmetric_ | |
| | CUAUAG-GGCACC | |
| 1021 | -5_6-6_internal_ | GAUAUUGUGACAUAUCUCUGCACUCAU |
| | loop-symmetric_ | CACACAAAAGAUGCAAAUCUUGAUCAA |
| | UCAGAG-AGACGU | GAUCCACAGGGAGGGGGCAUUUUAAU |
| | 26_6-6_internal_ | AUAUAGACGUACUAAUCAUC |
| | loop-symmetric_ | |
| | CUAUAG-GAUCAA | |
| 1022 | -5_6-6_internal_ | GAUAUUGUGACAUAUCUCUGCACUCAU |
| | loop-symmetric_ | CACACAAAAGAUGCAAAUCUGACAAUG |
| | UCAGAG-AACCAU | GAUCCACAGGGAGGGGGCAUUUUAAC |
| | 0_1-1_mismatch_ | AUAUAACCAUACUAAUCAUC |
| | A-C | |
| | 27_6-6_internal_ | |
| | loop-symmetric_ | |
| | UAUAGA-GACAAU | |
| 1023 | -5_6-6_internal_ | GAUAUUGUGACAUAUCUCUGCACUCAU |
| | loop-symmetric_ | CACACAAAAGAUGCAAAUCUAGCGCUG |
| | UCAGAG-AACAUU | GAUCCACAGGGAGGGGGCAUUUCAAU |
| | 3_1-1_mismatch_ | AUAUAACAUUACUAAUCAUC |
| | A-C | |
| | 27_6-6_internal_ | |
| | loop-symmetric_ | |
| | UAUAGA-AGCGCU | |
| 1024 | -5_6-6_internal_ | GAUAUUGUGACAUAUCUCUGCACUCAU |
| | loop-symmetric_ | CACACAAAAGAUGCAAAUCUCACGACG |
| | UCAGAG-GCAGUU | GAUCCACAGGGAGGGGGCAUUCUAAU |
| | 4_1-1_mismatch_ | AUAUGCAGUUACUAAUCAUC |
| | A-C | |
| | 27_6-6_internal_ | |
| | loop-symmetric_ | |
| | UAUAGA-CACGAC | |
| 1025 | -5_6-6_internal_ | GAUAUUGUGACAUAUCUCUGCACUCAU |
| | loop-symmetric_ | CACACAAAAGAUGCAAAUCUGUUUCCG |
| | UCAGAG-GGAGCC | GAUCCACAGGGAGGGGGCAUCUUAAU |
| | 5_1-1_mismatch_ | AUAUGGAGCCACUAAUCAUC |
| | A-C | |
| | 27_6-6_internal_ | |
| | loop-symmetric_ | |
| | UAUAGA-GUUUCC | |
| 1026 | -5_6-6_internal_ | GAUAUUGUGACAUAUCUCUGCACUCAU |
| | loop-symmetric_ | CACACAAAAGAUGCAAAUCUGGCUUUG |
| | UCAGAG-ACGAGC | GAUCCACAGGGAGGGGGCAUUUUAAU |
| | 27_6-6_internal_ | AUAUACGAGCACUAAUCAUC |
| | loop-symmetric_ | |
| | UAUAGA-GGCUUU | |
| 1027 | -5_6-6_internal_ | GAUAUUGUGACAUAUCUCUGCACUCAU |
| | loop-symmetric_ | CACACAAAAGAUGCAAAUCGACAGAAG |
| | UCAGAG-AAAGAU | GAUCCACAGGGAGGGGGCAUUUUAAC |
| | 0_1-1_mismatch_ | AUAUAAAGAUACUAAUCAUC |
| | A-C | |
| | 28_6-6_internal_ | |
| | loop-symmetric_ | |
| | AUAGAA-GACAGA | |
| 1028 | -5_6-6_internal_ | GAUAUUGUGACAUAUCUCUGCACUCAU |
| | loop-symmetric_ | CACACAAAAGAUGCAAAUCCUAGAGAG |
| | UCAGAG-AUAAAU | GAUCCACAGGGAGGGGGCAUUUCAAU |
| | 3_1-1_mismatch_ | AUAUAUAAAUACUAAUCAUC |
| | A-C | |
| | 28_6-6_internal_ | |
| | loop-symmetric_ | |
| | AUAGAA-CUAGAG | |
| 1029 | -5_6-6_internal_ | GAUAUUGUGACAUAUCUCUGCACUCAU |
| | loop-symmetric_ | CACACAAAAGAUGCAAAUCCUGCGGAG |
| | UCAGAG-AAGACU | GAUCCACAGGGAGGGGGCAUUCUAAU |
| | 4_1-1_mismatch_ | AUAUAAGACUACUAAUCAUC |
| | A-C | |
| | 28_6-6_internal_ | |
| | loop-symmetric_ | |
| | AUAGAA-CUGCGG | |
| 1030 | -5_6-6_internal_ | GAUAUUGUGACAUAUCUCUGCACUCAU |
| | loop-symmetric_ | CACACAAAAGAUGCAAAUCCGAUCGAG |
| | UCAGAG-GGUUAU | GAUCCACAGGGAGGGGGCAUCUUAAU |
| | 5_1-1_mismatch_ | AUAUGGUUAUACUAAUCAUC |
| | A-C | |
| | 28_6-6_internal_ | |
| | loop-symmetric_ | |
| | AUAGAA-CGAUCG | |
| 1031 | -5_6-6_internal_ | GAUAUUGUGACAUAUCUCUGCACUCAU |
| | loop-symmetric_ | CACACAAAAGAUGCAAAUCAAGUUCAG |
| | UCAGAG-AGAGCU | GAUCCACAGGGAGGGGGCAUUUUAAU |
| | 28_6-6_internal_ | AUAUAGAGCUACUAAUCAUC |
| | loop-symmetric_ | |
| | AUAGAA-AAGUUC | |
| 1032 | -5_6-6_internal_ | GAUAUUGUGACAUAUCUCUGCACUCAU |
| | loop-symmetric_ | CACACAAAAGAUGCAAAUAAAACCUAG |
| | UCAGAG-ACGUAC | GAUCCACAGGGAGGGGGCAUUUUAAC |
| | 0_1-1_mismatch_ | AUAUACGUACACUAAUCAUC |
| | A-C | |
| | 29_6-6_internal_ | |
| | loop-symmetric_ | |
| | UAGAAG-AAAACC | |
| 1033 | -5_6-6_internal_ | GAUAUUGUGACAUAUCUCUGCACUCAU |
| | loop-symmetric_ | CACACAAAAGAUGCAAAUGAGACUUA |
| | UCAGAG-AAACCU | GGAUCCACAGGGAGGGGGCAUUUCAA |
| | 3_1-1_mismatch_ | UAUAUAAACCUACUAAUCAUC |
| | A-C | |
| | 29_6-6_internal_ | |
| | loop-symmetric_ | |
| | UAGAAG-GAGACU | |
| 1034 | -5_6-6_internal_ | GAUAUUGUGACAUAUCUCUGCACUCAU |
| | loop-symmetric_ | CACACAAAAGAUGCAAAUGAAUGCUA |
| | UCAGAG-GCAUAU | GGAUCCACAGGGAGGGGGCAUCUUAA |
| | 5_1-1_mismatch_ | UAUAUGCAUAUACUAAUCAUC |
| | A-C | |
| | 29_6-6_internal_ | |
| | loop-symmetric_ | |
| | UAGAAG-GAAUGC | |
| 1035 | -5_6-6_internal_ | GAUAUUGUGACAUAUCUCUGCACUCAU |
| | loop-symmetric_ | CACACAAAAGAUGCAAAUGGCUUUUA |
| | UCAGAG-AAACGC | GGAUCCACAGGGAGGGGGCAUUUUAA |
| | 29_6-6_internal_ | UAUAUAAACGCACUAAUCAUC |
| | loop-symmetric_ | |
| | UAGAAG-GGCUUU | |
| 1036 | -5_6-6_internal_ | GAUAUUGUGACAUAUCUCUGCACUCAU |
| | loop-symmetric_ | CACACAAAAGAUGCAAACGAAUAAUA |
| | UCAGAG-GAUAUC | GGAUCCACAGGGAGGGGGCAUUUUAA |
| | 0_1-1_mismatch_ | CAUAUGAUAUCACUAAUCAUC |
| | A-C | |
| | 30_6-6_internal_ | |
| | loop-symmetric_ | |
| | AGAAGA-CGAAUA | |
| 1037 | -5_6-6_internal_ | GAUAUUGUGACAUAUCUCUGCACUCAU |
| | loop-symmetric_ | CACACAAAAGAUGCAAAAGCAACAUAG |
| | UCAGAG-GGACUU | GAUCCACAGGGAGGGGGCAUUUCAAU |
| | 3_1-1_mismatch_ | AUAUGGACUUACUAAUCAUC |
| | A-C | |
| | 30_6-6_internal_ | |
| | loop-symmetric_ | |
| | AGAAGA-AGCAAC | |
| 1038 | -5_6-6_internal_ | GAUAUUGUGACAUAUCUCUGCACUCAU |
| | loop-symmetric_ | CACACAAAAGAUGCAAAGGCAUAAUA |
| | UCAGAG-AAUUAU | GGAUCCACAGGGAGGGGGCAUUCUAA |
| | 4_1-1_mismatch_ | UAUAUAAUUAUACUAAUCAUC |
| | A-C | |
| | 30_6-6_internal_ | |
| | loop-symmetric_ | |
| | AGAAGA-GGCAUA | |
| 1039 | -5_6-6_internal_ | GAUAUUGUGACAUAUCUCUGCACUCAU |
| | loop-symmetric_ | CACACAAAAGAUGCAAACACCGGAUAG |
| | UCAGAG-GGAUAC | GAUCCACAGGGAGGGGGCAUCUUAAU |
| | 5_1-1_mismatch_ | AUAUGGAUACACUAAUCAUC |
| | A-C | |
| | 30_6-6_internal_ | |
| | loop-symmetric_ | |
| | AGAAGA-CACCGG | |
| 1040 | -5_6-6_internal_ | GAUAUUGUGACAUAUCUCUGCACUCAU |
| | loop-symmetric_ | CACACAAAAGAUGCAAACGAGAAAUA |
| | UCAGAG-GGGACC | GGAUCCACAGGGAGGGGGCAUUUUAA |
| | 30_6-6_internal_ | UAUAUGGGACCACUAAUCAUC |
| | loop-symmetric_ | |
| | AGAAGA-CGAGAA | |
| 1041 | -5_6-6_internal_ | GAUAUUGUGACAUAUCUCUGCACUCAU |
| | loop-symmetric_ | CACACAAAAGAUGCAACCUCAGUAUAG |
| | UCAGAG-GAACAC | GAUCCACAGGGAGGGGGCAUUUUAAC |
| | 0_1-1_mismatch_ | AUAUGAACACACUAAUCAUC |
| | A-C | |
| | 31_6-6_internal_ | |
| | loop-symmetric_ | |
| | GAAGAU-CCUCAG | |
| 1042 | -5_6-6_internal_ | GAUAUUGUGACAUAUCUCUGCACUCAU |
| | loop-symmetric_ | CACACAAAAGAUGCAACCAGCGUAUAG |
| | UCAGAG-GCAGAU | GAUCCACAGGGAGGGGGCAUUUCAAU |
| | 3_1-1_mismatch_ | AUAUGCAGAUACUAAUCAUC |
| | A-C | |
| | 31_6-6_internal_ | |
| | loop-symmetric_ | |
| | GAAGAU-CCAGCG | |
| 1043 | -5_6-6_internal_ | GAUAUUGUGACAUAUCUCUGCACUCAU |
| | loop-symmetric_ | CACACAAAAGAUGCAACGGAAAUAUA |
| | UCAGAG-GGAAUC | GGAUCCACAGGGAGGGGGCAUUCUAA |
| | 4_1-1_mismatch_ | UAUAUGGAAUCACUAAUCAUC |
| | A-C | |
| | 31_6-6_internal_ | |
| | loop-symmetric_ | |
| | GAAGAU-CGGAAA | |
| 1044 | -5_6-6_internal_ | GAUAUUGUGACAUAUCUCUGCACUCAU |
| | loop-symmetric_ | CACACAAAAGAUGCAAUUGGGAUAUA |
| | UCAGAG-GCAACU | GGAUCCACAGGGAGGGGGCAUCUUAA |
| | 5_1-1_mismatch_ | UAUAUGCAACUACUAAUCAUC |
| | A-C | |
| | 31_6-6_internal_ | |
| | loop-symmetric_ | |
| | GAAGAU-UUGGGA | |
| 1045 | -5_6-6_internal_ | GAUAUUGUGACAUAUCUCUGCACUCAU |
| | loop-symmetric_ | CACACAAAAGAUGCAACGUGCAUAUAG |
| | UCAGAG-AUGCAU | GAUCCACAGGGAGGGGGCAUUUUAAU |
| | 31_6-6_internal_ | AUAUAUGCAUACUAAUCAUC |
| | loop-symmetric_ | |
| | GAAGAU-CGUGCA | |
| 1046 | -5_6-6_internal_ | GAUAUUGUGACAUAUCUCUGCACUCAU |
| | loop-symmetric_ | CACACAAAAGAUGCAUUAAGCCUAUAG |
| | UCAGAG-ACAAGU | GAUCCACAGGGAGGGGGCAUUUUAAC |
| | 0_1-1_mismatch_ | AUAUACAAGUACUAAUCAUC |
| | A-C | |
| | 32_6-6_internal_ | |
| | loop-symmetric_ | |
| | AAGAUU-UUAAGC | |
| 1047 | -5_6-6_internal_ | GAUAUUGUGACAUAUCUCUGCACUCAU |
| | loop-symmetric_ | CACACAAAAGAUGCAUUAUAGCUAUA |
| | UCAGAG-AUAGUC | GGAUCCACAGGGAGGGGGCAUUUCAA |
| | 3_1-1_mismatch_ | UAUAUAUAGUCACUAAUCAUC |
| | A-C | |
| | 32_6-6_internal_ | |
| | loop-symmetric_ | |
| | AAGAUU-UUAUAG | |
| 1048 | -5_6-6_internal_ | GAUAUUGUGACAUAUCUCUGCACUCAU |
| | loop-symmetric_ | CACACAAAAGAUGCACUAGGACUAUAG |
| | UCAGAG-ACCAUU | GAUCCACAGGGAGGGGGCAUUCUAAU |
| | 4_1-1_mismatch_ | AUAUACCAUUACUAAUCAUC |
| | A-C | |
| | 32_6-6_internal_ | |
| | loop-symmetric_ | |
| | AAGAUU-CUAGGA | |
| 1049 | -5_6-6_internal_ | GAUAUUGUGACAUAUCUCUGCACUCAU |
| | loop-symmetric_ | CACACAAAAGAUGCAUAAGCCCUAUAG |
| | UCAGAG-GCAUUU | GAUCCACAGGGAGGGGGCAUCUUAAU |
| | 5_1-1_mismatch_ | AUAUGCAUUUACUAAUCAUC |
| | A-C | |
| | 32_6-6_internal_ | |
| | loop-symmetric_ | |
| | AAGAUU-UAAGCC | |
| 1050 | -5_6-6_internal_ | GAUAUUGUGACAUAUCUCUGCACUCAU |
| | loop-symmetric_ | CACACAAAAGAUGCUCGCCGUCUAUAG |
| | UCAGAG-AGUUAC | GAUCCACAGGGAGGGGGCAUUUUAAC |
| | 0_1-1_mismatch_ | AUAUAGUUACACUAAUCAUC |
| | A-C | |
| | 33_6-6_internal_ | |
| | loop-symmetric_ | |
| | AGAUUU-UCGCCG | |
| 1051 | -5_6-6_internal_ | GAUAUUGUGACAUAUCUCUGCACUCAU |
| | loop-symmetric_ | CACACAAAAGAUGCCGCCCCUCUAUAG |
| | UCAGAG-AGAUAC | GAUCCACAGGGAGGGGGCAUUUCAAU |
| | 3_1-1_mismatch_ | AUAUAGAUACACUAAUCAUC |
| | A-C | |
| | 33_6-6_internal_ | |
| | loop-symmetric_ | |
| | AGAUUU-CGCCCC | |
| 1052 | -5_6-6_internal_ | GAUAUUGUGACAUAUCUCUGCACUCAU |
| | loop-symmetric_ | CACACAAAAGAUGCUCUUGGUCUAUAG |
| | UCAGAG-AACAAC | GAUCCACAGGGAGGGGGCAUUCUAAU |
| | 4_1-1_mismatch_ | AUAUAACAACACUAAUCAUC |
| | A-C | |
| | 33_6-6_internal_ | |
| | loop-symmetric_ | |
| | AGAUUU-UCUUGG | |
| 1053 | -5_6-6_internal_ | GAUAUUGUGACAUAUCUCUGCACUCAU |
| | loop-symmetric_ | CACACAAAAGAUGCUCCAUAUCUAUAG |
| | UCAGAG-AGUAGC | GAUCCACAGGGAGGGGGCAUCUUAAU |
| | 5_1-1_mismatch_ | AUAUAGUAGCACUAAUCAUC |
| | A-C | |
| | 33_6-6_internal_ | |
| | loop-symmetric_ | |
| | AGAUUU-UCCAUA | |
| 1054 | -5_6-6_internal_ | GAUAUUGUGACAUAUCUCUGCACUCAU |
| | loop-symmetric_ | CACACAAAAGAUGCCCUGGAUCUAUAG |
| | UCAGAG-GAGAUC | GAUCCACAGGGAGGGGGCAUUUUAAU |
| | 33_6-6_internal_ | AUAUGAGAUCACUAAUCAUC |
| | loop-symmetric_ | |
| | AGAUUU-CCUGGA | |
| 1055 | -5_6-6_internal_ | GAUAUUGUGACAUAUCUCUGCACUCAU |
| | loop-symmetric_ | CACACAAAAGAUGGUCCUAUUCUAUAG |
| | UCAGAG-GACAUC | GAUCCACAGGGAGGGGGCAUUUUAAC |
| | 0_1-1_mismatch_ | AUAUGACAUCACUAAUCAUC |
| | A-C | |
| | 34_6-6_internal_ | |
| | loop-symmetric_ | |
| | GAUUUG-GUCCUA | |
| 1056 | -5_6-6_internal_ | GAUAUUGUGACAUAUCUCUGCACUCAU |
| | loop-symmetric_ | CACACAAAAGAUGGUCGCAUUCUAUAG |
| | UCAGAG-AAGAUC | GAUCCACAGGGAGGGGGCAUUUCAAU |
| | 3_1-1_mismatch_ | AUAUAAGAUCACUAAUCAUC |
| | A-C | |
| | 34_6-6_internal_ | |
| | loop-symmetric_ | |
| | GAUUUG-GUCGCA | |
| 1057 | -5_6-6_internal_ | GAUAUUGUGACAUAUCUCUGCACUCAU |
| | loop-symmetric_ | CACACAAAAGAUGGCCUCGUUCUAUAG |
| | UCAGAG-GGAGUU | GAUCCACAGGGAGGGGGCAUUCUAAU |
| | 4_1-1_mismatch_ | AUAUGGAGUUACUAAUCAUC |
| | A-C | |
| | 34_6-6_internal_ | |
| | loop-symmetric_ | |
| | GAUUUG-GCCUCG | |
| 1058 | -5_6-6_internal_ | GAUAUUGUGACAUAUCUCUGCACUCAU |
| | loop-symmetric_ | CACACAAAAGAUGAUCGGGUUCUAUA |
| | UCAGAG-ACGCAC | GGAUCCACAGGGAGGGGGCAUCUUAA |
| | 5_1-1_mismatch_ | UAUAUACGCACACUAAUCAUC |
| | A-C | |
| | 34_6-6_internal_ | |
| | loop-symmetric_ | |
| | GAUUUG-AUCGGG | |
| 1059 | -5_6-6_internal_ | GAUAUUGUGACAUAUCUCUGCACUCAU |
| | loop-symmetric_ | CACACAAAAGAUGGUUGGGUUCUAUA |
| | UCAGAG-AUACCC | GGAUCCACAGGGAGGGGGCAUUUUAA |
| | 34_6-6_internal_ | UAUAUAUACCCACUAAUCAUC |
| | loop-symmetric_ | |
| | GAUUUG-GUUGGG | |
| 1060 | -5_6-6_internal_ | GAUAUUGUGACAUAUCUCUGCACUCAU |
| | loop-symmetric_ | CACACAAAAGAUAAUUGACUUCUAUA |
| | UCAGAG-ACAAGU | GGAUCCACAGGGAGGGGGCAUUUUAA |
| | 0_1-1_mismatch_ | CAUAUACAAGUACUAAUCAUC |
| | A-C | |
| | 35_6-6_internal_ | |
| | loop-symmetric_ | |
| | AUUUGC-AAUUGA | |
| 1061 | -5_6-6_internal_ | GAUAUUGUGACAUAUCUCUGCACUCAU |
| | loop-symmetric_ | CACACAAAAGAUUAUUAGCUUCUAUA |
| | UCAGAG-GAAAGU | GGAUCCACAGGGAGGGGGCAUUUCAA |
| | 3_1-1_mismatch_ | UAUAUGAAAGUACUAAUCAUC |
| | A-C | |
| | 35_6-6_internal_ | |
| | loop-symmetric_ | |
| | AUUUGC-UAUUAG | |
| 1062 | -5_6-6_internal_ | GAUAUUGUGACAUAUCUCUGCACUCAU |
| | loop-symmetric_ | CACACAAAAGAUCUUCGCCUUCUAUAG |
| | UCAGAG-GAAGAU | GAUCCACAGGGAGGGGGCAUUCUAAU |
| | 4_1-1_mismatch_ | AUAUGAAGAUACUAAUCAUC |
| | A-C | |
| | 35_6-6_internal_ | |
| | loop-symmetric_ | |
| | AUUUGC-CUUCGC | |
| 1063 | -5_6-6_internal_ | GAUAUUGUGACAUAUCUCUGCACUCAU |
| | loop-symmetric_ | CACACAAAAGAUUGGUCCCUUCUAUAG |
| | UCAGAG-GAAGAU | GAUCCACAGGGAGGGGGCAUCUUAAU |
| | 5_1-1_mismatch_ | AUAUGAAGAUACUAAUCAUC |
| | A-C | |
| | 35_6-6_internal_ | |
| | loop-symmetric_ | |
| | AUUUGC-UGGUCC | |
| 1064 | -5_6-6_internal_ | GAUAUUGUGACAUAUCUCUGCACUCAU |
| | loop-symmetric_ | CACACAAAAGAUAGGUCGCUUCUAUAG |
| | UCAGAG-AUGGCU | GAUCCACAGGGAGGGGGCAUUUUAAU |
| | 35_6-6_internal_ | AUAUAUGGCUACUAAUCAUC |
| | loop-symmetric_ | |
| | AUUUGC-AGGUCG | |
| 1065 | -5_6-6_internal_ | GAUAUUGUGACAUAUCUCUGCACUCAU |
| | loop-symmetric_ | CACACAAAAGACCCCGUUCUUCUAUAG |
| | UCAGAG-GGGGUC | GAUCCACAGGGAGGGGGCAUUUUAAC |
| | 0_1-1_mismatch_ | AUAUGGGGUCACUAAUCAUC |
| | A-C | |
| | 36_6-6_internal_ | |
| | loop-symmetric_ | |
| | UUUGCA-CCCCGU | |
| 1066 | -5_6-6_internal_ | GAUAUUGUGACAUAUCUCUGCACUCAU |
| | loop-symmetric_ | CACACAAAAGACUCCUCUCUUCUAUAG |
| | UCAGAG-AAUUAC | GAUCCACAGGGAGGGGGCAUUUCAAU |
| | 3_1-1_mismatch_ | AUAUAAUUACACUAAUCAUC |
| | A-C | |
| | 36_6-6_internal_ | |
| | loop-symmetric_ | |
| | UUUGCA-CUCCUC | |
| 1067 | -5_6-6_internal_ | GAUAUUGUGACAUAUCUCUGCACUCAU |
| | loop-symmetric_ | CACACAAAAGAGAGACUUCUUCUAUAG |
| | UCAGAG-ACGUUU | GAUCCACAGGGAGGGGGCAUUCUAAU |
| | 4_1-1_mismatch_ | AUAUACGUUUACUAAUCAUC |
| | A-C | |
| | 36_6-6_internal_ | |
| | loop-symmetric_ | |
| | UUUGCA-GAGACU | |
| 1068 | -5_6-6_internal_ | GAUAUUGUGACAUAUCUCUGCACUCAU |
| | loop-symmetric_ | CACACAAAAGACCUCGUUCUUCUAUAG |
| | UCAGAG-GCACCC | GAUCCACAGGGAGGGGGCAUCUUAAU |
| | 5_1-1_mismatch_ | AUAUGCACCCACUAAUCAUC |
| | A-C | |
| | 36_6-6_internal_ | |
| | loop-symmetric_ | |
| | UUUGCA-CCUCGU | |
| 1069 | -5_6-6_internal_ | GAUAUUGUGACAUAUCUCUGCACUCAU |
| | loop-symmetric_ | CACACAAAAGAGAUUUCUCUUCUAUAG |
| | UCAGAG-ACGGUU | GAUCCACAGGGAGGGGGCAUUUUAAU |
| | 36_6-6_internal_ | AUAUACGGUUACUAAUCAUC |
| | loop-symmetric_ | |
| | UUUGCA-GAUUUC | |
| 1070 | -5_6-6_internal_ | GAUAUUGUGACAUAUCUCUGCACUCAU |
| | loop-symmetric_ | CACACAAAAGUCAGGUAUCUUCUAUAG |
| | UCAGAG-AGGACC | GAUCCACAGGGAGGGGGCAUUUUAAC |
| | 0_1-1_mismatch_ | AUAUAGGACCACUAAUCAUC |
| | A-C | |
| | 37_6-6_internal_ | |
| | loop-symmetric_ | |
| | UUGCAU-UCAGGU | |
| 1071 | -5_6-6_internal_ | GAUAUUGUGACAUAUCUCUGCACUCAU |
| | loop-symmetric_ | CACACAAAAGCGAUUCAUCUUCUAUAG |
| | UCAGAG-AAGGUU | GAUCCACAGGGAGGGGGCAUUUCAAU |
| | 3_1-1_mismatch_ | AUAUAAGGUUACUAAUCAUC |
| | A-C | |
| | 37_6-6_internal_ | |
| | loop-symmetric_ | |
| | UUGCAU-CGAUUC | |
| 1072 | -5_6-6_internal_ | GAUAUUGUGACAUAUCUCUGCACUCAU |
| | loop-symmetric_ | CACACAAAAGCUCGUCAUCUUCUAUAG |
| | UCAGAG-GGAGAU | GAUCCACAGGGAGGGGGCAUUCUAAU |
| | 4_1-1_mismatch_ | AUAUGGAGAUACUAAUCAUC |
| | A-C | |
| | 37_6-6_internal_ | |
| | loop-symmetric_ | |
| | UUGCAU-CUCGUC | |
| 1073 | -5_6-6_internal_ | GAUAUUGUGACAUAUCUCUGCACUCAU |
| | loop-symmetric_ | CACACAAAAGUCCGUCAUCUUCUAUAG |
| | UCAGAG-GAGCCC | GAUCCACAGGGAGGGGGCAUCUUAAU |
| | 5_1-1_mismatch_ | AUAUGAGCCCACUAAUCAUC |
| | A-C | |
| | 37_6-6_internal_ | |
| | loop-symmetric_ | |
| | UUGCAU-UCCGUC | |
| 1074 | -5_6-6_internal_ | GAUAUUGUGACAUAUCUCUGCACUCAU |
| | loop-symmetric_ | CACACAAAAGCCAUCUAUCUUCUAUAG |
| | UCAGAG-ACCAUC | GAUCCACAGGGAGGGGGCAUUUUAAU |
| | 37_6-6_internal_ | AUAUACCAUCACUAAUCAUC |
| | loop-symmetric_ | |
| | UUGCAU-CCAUCU | |
| 1075 | -5_6-6_internal_ | GAUAUUGUGACAUAUCUCUGCACUCAU |
| | loop-symmetric_ | CACACAAAAUCUCGUAAUCUUCUAUAG |
| | UCAGAG-GCAAUU | GAUCCACAGGGAGGGGGCAUUUUAAC |
| | 0_1-1_mismatch_ | AUAUGCAAUUACUAAUCAUC |
| | A-C | |
| | 38_6-6_internal_ | |
| | loop-symmetric_ | |
| | UGCAUC-UCUCGU | |
| 1076 | -5_6-6_internal_ | GAUAUUGUGACAUAUCUCUGCACUCAU |
| | loop-symmetric_ | CACACAAAAACCCAUAAUCUUCUAUAG |
| | UCAGAG-AGAUAU | GAUCCACAGGGAGGGGGCAUUUCAAU |
| | 3_1-1_mismatch_ | AUAUAGAUAUACUAAUCAUC |
| | A-C | |
| | 38_6-6_internal_ | |
| | loop-symmetric_ | |
| | UGCAUC-ACCCAU | |
| 1077 | -5_6-6_internal_ | GAUAUUGUGACAUAUCUCUGCACUCAU |
| | loop-symmetric_ | CACACAAAACUACUCAAUCUUCUAUAG |
| | UCAGAG-AUACGU | GAUCCACAGGGAGGGGGCAUUCUAAU |
| | 4_1-1_mismatch_ | AUAUAUACGUACUAAUCAUC |
| | A-C | |
| | 38_6-6_internal_ | |
| | loop-symmetric_ | |
| | UGCAUC-CUACUC | |
| 1078 | -5_6-6_internal_ | GAUAUUGUGACAUAUCUCUGCACUCAU |
| | loop-symmetric_ | CACACAAAAACGUUUAAUCUUCUAUAG |
| | UCAGAG-GGAGAU | GAUCCACAGGGAGGGGGCAUCUUAAU |
| | 5_1-1_mismatch_ | AUAUGGAGAUACUAAUCAUC |
| | A-C | |
| | 38_6-6_internal_ | |
| | loop-symmetric_ | |
| | UGCAUC-ACGUUU | |
| 1079 | -5_6-6_internal_ | GAUAUUGUGACAUAUCUCUGCACUCAU |
| | loop-symmetric_ | CACACAAAUUCUAGAAAUCUUCUAUAG |
| | UCAGAG-AAAUUU | GAUCCACAGGGAGGGGGCAUUUCAAU |
| | 3_1-1_mismatch_ | AUAUAAAUUUACUAAUCAUC |
| | A-C | |
| | 39_6-6_internal_ | |
| | loop-symmetric_ | |
| | GCAUCU-UUCUAG | |
| 1080 | -5_6-6_internal_ | GAUAUUGUGACAUAUCUCUGCACUCAU |
| | loop-symmetric_ | CACACAAAUCAAAAAAAUCUUCUAUAG |
| | UCAGAG-AAGGCC | GAUCCACAGGGAGGGGGCAUUCUAAU |
| | 4_1-1_mismatch_ | AUAUAAGGCCACUAAUCAUC |
| | A-C | |
| | 39_6-6_internal_ | |
| | loop-symmetric_ | |
| | GCAUCU-UCAAAA | |
| 1081 | -5_6-6_internal_ | GAUAUUGUGACAUAUCUCUGCACUCAU |
| | loop-symmetric_ | CACACAAACGCCCAAAAUCUUCUAUAG |
| | UCAGAG-AAGUUU | GAUCCACAGGGAGGGGGCAUCUUAAU |
| | 5_1-1_mismatch_ | AUAUAAGUUUACUAAUCAUC |
| | A-C | |
| | 39_6-6_internal_ | |
| | loop-symmetric_ | |
| | GCAUCU-CGCCCA | |
| 1082 | -5_6-6_internal_ | GAUAUUGUGACAUAUCUCUGCACUCAU |
| | loop-symmetric_ | CACACAAACAUCAGAAAUCUUCUAUAG |
| | UCAGAG-GAUCAC | GAUCCACAGGGAGGGGGCAUUUUAAU |
| | 39_6-6_internal_ | AUAUGAUCACACUAAUCAUC |
| | loop-symmetric_ | |
| | GCAUCU-CAUCAG | |
| 1083 | -5_6-6_internal_ | GAUAUUGUGACAUAUCUCUGCACUCAU |
| | loop-symmetric_ | CACACAAUCCAGUCAAAUCUUCUAUAG |
| | UCAGAG-AGAGUC | GAUCCACAGGGAGGGGGCAUUUUAAC |
| | 0_1-1_mismatch_ | AUAUAGAGUCACUAAUCAUC |
| | A-C | |
| | 40_6-6_internal_ | |
| | loop-symmetric_ | |
| | CAUCUU-UCCAGU | |
| 1084 | -5_6-6_internal_ | GAUAUUGUGACAUAUCUCUGCACUCAU |
| | loop-symmetric_ | CACACAAUCUGACCAAAUCUUCUAUAG |
| | UCAGAG-AAGUCU | GAUCCACAGGGAGGGGGCAUUUCAAU |
| | 3_1-1_mismatch_ | AUAUAAGUCUACUAAUCAUC |
| | A-C | |
| | 40_6-6_internal_ | |
| | loop-symmetric_ | |
| | CAUCUU-UCUGAC | |
| 1085 | -5_6-6_internal_ | GAUAUUGUGACAUAUCUCUGCACUCAU |
| | loop-symmetric_ | CACACAAUUACUUCAAAUCUUCUAUAG |
| | UCAGAG-AUGCCU | GAUCCACAGGGAGGGGGCAUUCUAAU |
| | 4_1-1_mismatch_ | AUAUAUGCCUACUAAUCAUC |
| | A-C | |
| | 40_6-6_internal_ | |
| | loop-symmetric_ | |
| | CAUCUU-UUACUU | |
| 1086 | -5_6-6_internal_ | GAUAUUGUGACAUAUCUCUGCACUCAU |
| | loop-symmetric_ | CACACAACCUAACCAAAUCUUCUAUAG |
| | UCAGAG-AAACCC | GAUCCACAGGGAGGGGGCAUCUUAAU |
| | 5_1-1_mismatch_ | AUAUAAACCCACUAAUCAUC |
| | A-C | |
| | 40_6-6_internal_ | |
| | loop-symmetric_ | |
| | CAUCUU-CCUAAC | |
| 1087 | -5_6-6_internal_ | GAUAUUGUGACAUAUCUCUGCACUCAU |
| | loop-symmetric_ | CACACAAUCUUACCAAAUCUUCUAUAG |
| | UCAGAG-GCAAUU | GAUCCACAGGGAGGGGGCAUUUUAAU |
| | 40_6-6_internal_ | AUAUGCAAUUACUAAUCAUC |
| | loop-symmetric_ | |
| | CAUCUU-UCUUAC | |
| 1088 | -5_6-6_internal_ | GAUAUUGUGACAUAUCUCUGCACUCAU |
| | loop-symmetric_ | CACACAUUGUCAGCAAAUCUUCUAUAG |
| | UCAGAG-ACGAAU | GAUCCACAGGGAGGGGGCAUUUUAAC |
| | 0_1-1_mismatch_ | AUAUACGAAUACUAAUCAUC |
| | A-C | |
| | 41_6-6_internal_ | |
| | loop-symmetric_ | |
| | AUCUUU-UUGUCA | |
| 1089 | -5_6-6_internal_ | GAUAUUGUGACAUAUCUCUGCACUCAU |
| | loop-symmetric_ | CACACACCCAUCGCAAAUCUUCUAUAG |
| | UCAGAG-GGGGUC | GAUCCACAGGGAGGGGGCAUUUCAAU |
| | 3_1-1_mismatch_ | AUAUGGGGUCACUAAUCAUC |
| | A-C | |
| | 41_6-6_internal_ | |
| | loop-symmetric_ | |
| | AUCUUU-CCCAUC | |
| 1090 | -5_6-6_internal_ | GAUAUUGUGACAUAUCUCUGCACUCAU |
| | loop-symmetric_ | CACACAUUGUCGGCAAAUCUUCUAUAG |
| | UCAGAG-AGAAUU | GAUCCACAGGGAGGGGGCAUUCUAAU |
| | 4_1-1_mismatch_ | AUAUAGAAUUACUAAUCAUC |
| | A-C | |
| | 41_6-6_internal_ | |
| | loop-symmetric_ | |
| | AUCUUU-UUGUCG | |
| 1091 | -5_6-6_internal_ | GAUAUUGUGACAUAUCUCUGCACUCAU |
| | loop-symmetric_ | CACACAUCCACAGCAAAUCUUCUAUAG |
| | UCAGAG-GGAGCC | GAUCCACAGGGAGGGGGCAUCUUAAU |
| | 5_1-1_mismatch_ | AUAUGGAGCCACUAAUCAUC |
| | A-C | |
| | 41_6-6_internal_ | |
| | loop-symmetric_ | |
| | AUCUUU-UCCACA | |
| 1092 | -5_6-6_internal_ | GAUAUUGUGACAUAUCUCUGCACUCAU |
| | loop-symmetric_ | CACACACGCAUGGCAAAUCUUCUAUAG |
| | UCAGAG-AGAAAU | GAUCCACAGGGAGGGGGCAUUUUAAU |
| | 41_6-6_internal_ | AUAUAGAAAUACUAAUCAUC |
| | loop-symmetric_ | |
| | AUCUUU-CGCAUG | |
| 1093 | -5_6-6_internal_ | GAUAUUGUGACAUAUCUCUGCACUCAU |
| | loop-symmetric_ | CACACUCAUCUUGCAAAUCUUCUAUAG |
| | UCAGAG-ACACAU | GAUCCACAGGGAGGGGGCAUUUUAAC |
| | 0_1-1_mismatch_ | AUAUACACAUACUAAUCAUC |
| | A-C | |
| | 42_6-6_internal_ | |
| | loop-symmetric_ | |
| | UCUUUU-UCAUCU | |
| 1094 | -5_6-6_internal_ | GAUAUUGUGACAUAUCUCUGCACUCAU |
| | loop-symmetric_ | CACACUUUAACUGCAAAUCUUCUAUAG |
| | UCAGAG-GACCAC | GAUCCACAGGGAGGGGGCAUUUCAAU |
| | 3_1-1_mismatch_ | AUAUGACCACACUAAUCAUC |
| | A-C | |
| | 42_6-6_internal_ | |
| | loop-symmetric_ | |
| | UCUUUU-UUUAAC | |
| 1095 | -5_6-6_internal_ | GAUAUUGUGACAUAUCUCUGCACUCAU |
| | loop-symmetric_ | CACACCUCUAUUGCAAAUCUUCUAUAG |
| | UCAGAG-GAUAUU | GAUCCACAGGGAGGGGGCAUUCUAAU |
| | 4_1-1_mismatch_ | AUAUGAUAUUACUAAUCAUC |
| | A-C | |
| | 42_6-6_internal_ | |
| | loop-symmetric_ | |
| | UCUUUU-CUCUAU | |
| 1096 | -5_6-6_internal_ | GAUAUUGUGACAUAUCUCUGCACUCAU |
| | loop-symmetric_ | CACACUGCCACUGCAAAUCUUCUAUAG |
| | UCAGAG-GGACAC | GAUCCACAGGGAGGGGGCAUCUUAAU |
| | 5_1-1_mismatch_ | AUAUGGACACACUAAUCAUC |
| | A-C | |
| | 42_6-6_internal_ | |
| | loop-symmetric_ | |
| | UCUUUU-UGCCAC | |
| 1097 | -5_6-6_internal_ | GAUAUUGUGACAUAUCUCUGCACUCAU |
| | loop-symmetric_ | CACACUGCUUUUGCAAAUCUUCUAUAG |
| | UCAGAG-AAUAUU | GAUCCACAGGGAGGGGGCAUUUUAAU |
| | 42_6-6_internal_ | AUAUAAUAUUACUAAUCAUC |
| | loop-symmetric_ | |
| | UCUUUU-UGCUUU | |
| 1098 | -5_6-6_internal_ | GAUAUUGUGACAUAUCUCUGCACUCAU |
| | loop-symmetric_ | CACAGCACCCAUGCAAAUCUUCUAUAG |
| | UCAGAG-GCGACC | GAUCCACAGGGAGGGGGCAUUUUAAC |
| | 0_1-1_mismatch_ | AUAUGCGACCACUAAUCAUC |
| | A-C | |
| | 43_6-6_internal_ | |
| | loop-symmetric_ | |
| | CUUUUG-GCACCC | |
| 1099 | -5_6-6_internal_ | GAUAUUGUGACAUAUCUCUGCACUCAU |
| | loop-symmetric_ | CACAAGGUCUAUGCAAAUCUUCUAUAG |
| | UCAGAG-GCGUCC | GAUCCACAGGGAGGGGGCAUUUCAAU |
| | 3_1-1_mismatch_ | AUAUGCGUCCACUAAUCAUC |
| | A-C | |
| | 43_6-6_internal_ | |
| | loop-symmetric_ | |
| | CUUUUG-AGGUCU | |
| 1100 | -5_6-6_internal_ | GAUAUUGUGACAUAUCUCUGCACUCAU |
| | loop-symmetric_ | CACAGUAUACAUGCAAAUCUUCUAUAG |
| | UCAGAG-GCGUUU | GAUCCACAGGGAGGGGGCAUUCUAAU |
| | 4_1-1_mismatch_ | AUAUGCGUUUACUAAUCAUC |
| | A-C | |
| | 43_6-6_internal_ | |
| | loop-symmetric_ | |
| | CUUUUG-GUAUAC | |
| 1101 | -5_6-6_internal_ | GAUAUUGUGACAUAUCUCUGCACUCAU |
| | loop-symmetric_ | CACAGUCUAAAUGCAAAUCUUCUAUAG |
| | UCAGAG-GGUUAC | GAUCCACAGGGAGGGGGCAUCUUAAU |
| | 5_1-1_mismatch_ | AUAUGGUUACACUAAUCAUC |
| | A-C | |
| | 43_6-6_internal_ | |
| | loop-symmetric_ | |
| | CUUUUG-GUCUAA | |
| 1102 | -5_6-6_internal_ | GAUAUUGUGACAUAUCUCUGCACUCAU |
| | loop-symmetric_ | CACAGUUGCCAUGCAAAUCUUCUAUAG |
| | UCAGAG-AUACGC | GAUCCACAGGGAGGGGGCAUUUUAAU |
| | 43_6-6_internal_ | AUAUAUACGCACUAAUCAUC |
| | loop-symmetric_ | |
| | CUUUUG-GUUGCC | |
| 1103 | -5_6-6_internal_ | GAUAUUGUGACAUAUCUCUGCACUCAU |
| | loop-symmetric_ | CACCUACUCGAUGCAAAUCUUCUAUAG |
| | UCAGAG-GCAGCU | GAUCCACAGGGAGGGGGCAUUUUAAC |
| | 0_1-1_mismatch_ | AUAUGCAGCUACUAAUCAUC |
| | A-C | |
| | 44_6-6_internal_ | |
| | loop-symmetric_ | |
| | UUUUGU-CUACUC | |
| 1104 | -5_6-6_internal_ | GAUAUUGUGACAUAUCUCUGCACUCAU |
| | loop-symmetric_ | CACUGCCCCGAUGCAAAUCUUCUAUAG |
| | UCAGAG-GGGUCC | GAUCCACAGGGAGGGGGCAUUUCAAU |
| | 3_1-1_mismatch_ | AUAUGGGUCCACUAAUCAUC |
| | A-C | |
| | 44_6-6_internal_ | |
| | loop-symmetric_ | |
| | UUUUGU-UGCCCC | |
| 1105 | -5_6-6_internal_ | GAUAUUGUGACAUAUCUCUGCACUCAU |
| | loop-symmetric_ | CACUGCCUUGAUGCAAAUCUUCUAUAG |
| | UCAGAG-GCGCAU | GAUCCACAGGGAGGGGGCAUCUUAAU |
| | 5_1-1_mismatch_ | AUAUGCGCAUACUAAUCAUC |
| | A-C | |
| | 44_6-6_internal_ | |
| | loop-symmetric_ | |
| | UUUUGU-UGCCUU | |
| 1106 | -5_6-6_internal_ | GAUAUUGUGACAUAUCUCUGCACUCAU |
| | loop-symmetric_ | CACCUGGUUGAUGCAAAUCUUCUAUAG |
| | UCAGAG-GGGCAC | GAUCCACAGGGAGGGGGCAUUUUAAU |
| | 44_6-6_internal_ | AUAUGGGCACACUAAUCAUC |
| | loop-symmetric_ | |
| | UUUUGU-CUGGUU | |
| 1107 | -4_6-6_internal_ | GAUAUUGUGACAUAUCUCUGCACUCAU |
| | loop-symmetric_ | CACACAAAAGAUGCAAAUCUUCUAUAG |
| | CAGAGA-GUAAGA | GAUCCACAGGGAGGGGGCAUUUUAAC |
| | 0_1-1_mismatch_ | AUAGUAAGAAACUAAUCAUC |
| | A-C | |
| 1108 | -4_6-6_internal_ | GAUAUUGUGACAUAUCUCUGCACUCAU |
| | loop-symmetric_ | CACACAAAAGAUGCAAAUCUUCUAUAG |
| | CAGAGA-GCCCAU | GAUCCACAGGGAGGGGGCAUUUCAAU |
| | 3_1-1_mismatch_ | AUAGCCCAUAACUAAUCAUC |
| | A-C | |
| 1109 | -4_6-6_internal_ | GAUAUUGUGACAUAUCUCUGCACUCAU |
| | loop-symmetric_ | CACACAAAAGAUGCAAAUCUUCUAUAG |
| | CAGAGA-CGAGAU | GAUCCACAGGGAGGGGGCAUUCUAAU |
| | 4_1-1_mismatch_ | AUACGAGAUAACUAAUCAUC |
| | A-C | |
| 1110 | -4_6-6_internal_ | GAUAUUGUGACAUAUCUCUGCACUCAU |
| | loop-symmetric_ | CACACAAAAGAUGCAAAUCUUCUAUAG |
| | CAGAGA-AUGUAC | GAUCCACAGGGAGGGGGCAUCUUAAU |
| | 5_1-1_mismatch_ | AUAAUGUACAACUAAUCAUC |
| | A-C | |
| 1111 | -4_6-6_internal_ | GAUAUUGUGACAUAUCUCUGCACUCAU |
| | loop-symmetric_ | CACACAAAAGAUGCAAAUCUUCUAUAG |
| | CAGAGA-AGAGAC | GAUCCACAGGGAGGGGGCAUUUUAAU |
| | | AUAAGAGACAACUAAUCAUC |
| 1112 | -4_6-6_internal_ | GAUAUUGUGACAUAUCUCUGCACUCAU |
| | loop-symmetric_ | CACACAAAAGAUGCAAAUCUUCUAUCC |
| | CAGAGA-GUACGC | AUGACACAGGGAGGGGGCAUUUUAAC |
| | 0_1-1_mismatch_ | AUAGUACGCAACUAAUCAUC |
| | A-C | |
| | 22_6-6_internal_ | |
| | loop-symmetric_ | |
| | GAUCCU-CCAUGA | |
| 1113 | -4_6-6_internal_ | GAUAUUGUGACAUAUCUCUGCACUCAU |
| | loop-symmetric_ | CACACAAAAGAUGCAAAUCUUCUAUUU |
| | CAGAGA-AGAGCC | UUGACACAGGGAGGGGGCAUUUCAAU |
| | 3_1-1_mismatch_ | AUAAGAGCCAACUAAUCAUC |
| | A-C | |
| | 22_6-6_internal_ | |
| | loop-symmetric_ | |
| | GAUCCU-UUUUGA | |
| 1114 | -4_6-6_internal_ | GAUAUUGUGACAUAUCUCUGCACUCAU |
| | loop-symmetric_ | CACACAAAAGAUGCAAAUCUUCUAUCC |
| | CAGAGA-AGCACC | AUGGCACAGGGAGGGGGCAUUCUAAU |
| | 4_1-1_mismatch_ | AUAAGCACCAACUAAUCAUC |
| | A-C | |
| | 22_6-6_internal_ | |
| | loop-symmetric_ | |
| | GAUCCU-CCAUGG | |
| 1115 | -4_6-6_internal_ | GAUAUUGUGACAUAUCUCUGCACUCAU |
| | loop-symmetric_ | CACACAAAAGAUGCAAAUCUUCUAUUC |
| | CAGAGA-AGCAGC | UUGGCACAGGGAGGGGGCAUCUUAAU |
| | 5_1-1_mismatch_ | AUAAGCAGCAACUAAUCAUC |
| | A-C | |
| | 22_6-6_internal_ | |
| | loop-symmetric_ | |
| | GAUCCU-UCUUGG | |
| 1116 | -4_6-6_internal_ | GAUAUUGUGACAUAUCUCUGCACUCAU |
| | loop-symmetric_ | CACACAAAAGAUGCAAAUCUUCUAUCC |
| | CAGAGA-CGAAAC | CCAGCACAGGGAGGGGGCAUUUUAAU |
| | 22_6-6_internal_ | AUACGAAACAACUAAUCAUC |
| | loop-symmetric_ | |
| | GAUCCU-CCCCAG | |
| 1117 | -4_6-6_internal_ | GAUAUUGUGACAUAUCUCUGCACUCAU |
| | loop-symmetric_ | CACACAAAAGAUGCAAAUCUUCUACCU |
| | CAGAGA-GUAAUC | UUCCCACAGGGAGGGGGCAUUUUAACA |
| | 0_1-1_mismatch_ | UAGUAAUCAACUAAUCAUC |
| | A-C | |
| | 23_6-6_internal_ | |
| | loop-symmetric_ | |
| | AUCCUA-CCUUUC | |
| 1118 | -4_6-6_internal_ | GAUAUUGUGACAUAUCUCUGCACUCAU |
| | loop-symmetric_ | CACACAAAAGAUGCAAAUCUUCUACGA |
| | CAGAGA-CAGAGA | AACCCACAGGGAGGGGGCAUUUCAAUA |
| | 3_1-1_mismatch_ | UACAGAGAAACUAAUCAUC |
| | A-C | |
| | 23_6-6_internal_ | |
| | loop-symmetric_ | |
| | AUCCUA-CGAAAC | |
| 1119 | -4_6-6_internal_ | GAUAUUGUGACAUAUCUCUGCACUCAU |
| | loop-symmetric_ | CACACAAAAGAUGCAAAUCUUCUACUC |
| | CAGAGA-AACGGA | GUACCACAGGGAGGGGGCAUUCUAAU |
| | 4_1-1_mismatch_ | AUAAACGGAAACUAAUCAUC |
| | A-C | |
| | 23_6-6_internal_ | |
| | loop-symmetric_ | |
| | AUCCUA-CUCGUA | |
| 1120 | -4_6-6_internal_ | GAUAUUGUGACAUAUCUCUGCACUCAU |
| | loop-symmetric_ | CACACAAAAGAUGCAAAUCUUCUAACA |
| | CAGAGA-AGCAGU | ACACCACAGGGAGGGGGCAUCUUAAUA |
| | 5_1-1_mismatch_ | UAAGCAGUAACUAAUCAUC |
| | A-C | |
| | 23_6-6_internal_ | |
| | loop-symmetric_ | |
| | AUCCUA-ACAACA | |
| 1121 | -4_6-6_internal_ | GAUAUUGUGACAUAUCUCUGCACUCAU |
| | loop-symmetric_ | CACACAAAAGAUGCAAAUCUUCUACUC |
| | CAGAGA-GAGGUA | UCGCCACAGGGAGGGGGCAUUUUAAU |
| | 23_6-6_internal_ | AUAGAGGUAAACUAAUCAUC |
| | loop-symmetric_ | |
| | AUCCUA-CUCUCG | |
| 1122 | -4_6-6_internal_ | GAUAUUGUGACAUAUCUCUGCACUCAU |
| | loop-symmetric_ | CACACAAAAGAUGCAAAUCUUCUUACA |
| | CAGAGA-GAGGUA | UUUCCACAGGGAGGGGGCAUUUUAAC |
| | 0_1-1_mismatch_ | AUAGAGGUAAACUAAUCAUC |
| | A-C | |
| | 24_6-6_internal_ | |
| | loop-symmetric_ | |
| | UCCUAU-UACAUU | |
| 1123 | -4_6-6_internal_ | GAUAUUGUGACAUAUCUCUGCACUCAU |
| | loop-symmetric_ | CACACAAAAGAUGCAAAUCUUCUUAAU |
| | CAGAGA-GCAGGA | CCUCCACAGGGAGGGGGCAUUUCAAUA |
| | 3_1-1_mismatch_ | UAGCAGGAAACUAAUCAUC |
| | A-C | |
| | 24_6-6_internal_ | |
| | loop-symmetric_ | |
| | UCCUAU-UAAUCC | |
| 1124 | -4_6-6_internal_ | GAUAUUGUGACAUAUCUCUGCACUCAU |
| | loop-symmetric_ | CACACAAAAGAUGCAAAUCUUCUCAGA |
| | CAGAGA-AACGUC | AUUCCACAGGGAGGGGGCAUUCUAAU |
| | 4_1-1_mismatch_ | AUAAACGUCAACUAAUCAUC |
| | A-C | |
| | 24_6-6_internal_ | |
| | loop-symmetric_ | |
| | UCCUAU-CAGAAU | |
| 1125 | -4_6-6_internal_ | GAUAUUGUGACAUAUCUCUGCACUCAU |
| | loop-symmetric_ | CACACAAAAGAUGCAAAUCUUCUCCGU |
| | CAGAGA-GGCGUU | UCUCCACAGGGAGGGGGCAUCUUAAUA |
| | 5_1-1_mismatch_ | UAGGCGUUAACUAAUCAUC |
| | A-C | |
| | 24_6-6_internal_ | |
| | loop-symmetric_ | |
| | UCCUAU-CCGUUC | |
| 1126 | -4_6-6_internal_ | GAUAUUGUGACAUAUCUCUGCACUCAU |
| | loop-symmetric_ | CACACAAAAGAUGCAAAUCUUCUCCCU |
| | CAGAGA-AGGAGU | CUUCCACAGGGAGGGGGCAUUUUAAU |
| | 24_6-6_internal_ | AUAAGGAGUAACUAAUCAUC |
| | loop-symmetric_ | |
| | UCCUAU-CCCUCU | |
| 1127 | -4_6-6_internal_ | GAUAUUGUGACAUAUCUCUGCACUCAU |
| | loop-symmetric_ | CACACAAAAGAUGCAAAUCUUCAGAUU |
| | CAGAGA-CGGACA | CAUCCACAGGGAGGGGGCAUUUUAACA |
| | 0_1-1_mismatch_ | UACGGACAAACUAAUCAUC |
| | A-C | |
| | 25_6-6_internal_ | |
| | loop-symmetric_ | |
| | CCUAUA-AGAUUC | |
| 1128 | -4_6-6_internal_ | GAUAUUGUGACAUAUCUCUGCACUCAU |
| | loop-symmetric_ | CACACAAAAGAUGCAAAUCUUCACUCC |
| | CAGAGA-AGGAAU | UAUCCACAGGGAGGGGGCAUUUCAAU |
| | 3_1-1_mismatch_ | AUAAGGAAUAACUAAUCAUC |
| | A-C | |
| | 25_6-6_internal_ | |
| | loop-symmetric_ | |
| | CCUAUA-ACUCCU | |
| 1129 | -4_6-6_internal_ | GAUAUUGUGACAUAUCUCUGCACUCAU |
| | loop-symmetric_ | CACACAAAAGAUGCAAAUCUUCGUCGA |
| | CAGAGA-GUACGC | UAUCCACAGGGAGGGGGCAUUCUAAU |
| | 4_1-1_mismatch_ | AUAGUACGCAACUAAUCAUC |
| | A-C | |
| | 25_6-6_internal_ | |
| | loop-symmetric_ | |
| | CCUAUA-GUCGAU | |
| 1130 | -4_6-6_internal_ | GAUAUUGUGACAUAUCUCUGCACUCAU |
| | loop-symmetric_ | CACACAAAAGAUGCAAAUCUUCACGUC |
| | CAGAGA-AGGAGU | AAUCCACAGGGAGGGGGCAUCUUAAU |
| | 5_1-1_mismatch_ | AUAAGGAGUAACUAAUCAUC |
| | A-C | |
| | 25_6-6_internal_ | |
| | loop-symmetric_ | |
| | CCUAUA-ACGUCA | |
| 1131 | -4_6-6_internal_ | GAUAUUGUGACAUAUCUCUGCACUCAU |
| | loop-symmetric_ | CACACAAAAGAUGCAAAUCUUCCCACU |
| | CAGAGA-GACAGC | CAUCCACAGGGAGGGGGCAUUUUAAU |
| | 25_6-6_internal_ | AUAGACAGCAACUAAUCAUC |
| | loop-symmetric_ | |
| | CCUAUA-CCACUC | |
| 1132 | -4_6-6_internal_ | GAUAUUGUGACAUAUCUCUGCACUCAU |
| | loop-symmetric_ | CACACAAAAGAUGCAAAUCUUGCUCCU |
| | CAGAGA-GAAAAA | GAUCCACAGGGAGGGGGCAUUUUAAC |
| | 0_1-1_mismatch_ | AUAGAAAAAAACUAAUCAUC |
| | A-C | |
| | 26_6-6_internal_ | |
| | loop-symmetric_ | |
| | CUAUAG-GCUCCU | |
| 1133 | -4_6-6_internal_ | GAUAUUGUGACAUAUCUCUGCACUCAU |
| | loop-symmetric_ | CACACAAAAGAUGCAAAUCUUAAUCGA |
| | CAGAGA-CGGACC | GAUCCACAGGGAGGGGGCAUUUCAAU |
| | 3_1-1_mismatch_ | AUACGGACCAACUAAUCAUC |
| | A-C | |
| | 26_6-6_internal_ | |
| | loop-symmetric_ | |
| | CUAUAG-AAUCGA | |
| 1134 | -4_6-6_internal_ | GAUAUUGUGACAUAUCUCUGCACUCAU |
| | loop-symmetric_ | CACACAAAAGAUGCAAAUCUUGCGGUU |
| | CAGAGA-GGUGCA | GAUCCACAGGGAGGGGGCAUUCUAAU |
| | 4_1-1_mismatch_ | AUAGGUGCAAACUAAUCAUC |
| | A-C | |
| | 26_6-6_internal_ | |
| | loop-symmetric_ | |
| | CUAUAG-GCGGUU | |
| 1135 | -4_6-6_internal_ | GAUAUUGUGACAUAUCUCUGCACUCAU |
| | loop-symmetric_ | CACACAAAAGAUGCAAAUCUUAAAAU |
| | CAGAGA-AAGGGC | AGAUCCACAGGGAGGGGGCAUCUUAA |
| | 5_1-1_mismatch_ | UAUAAAGGGCAACUAAUCAUC |
| | A-C | |
| | 26_6-6_internal_ | |
| | loop-symmetric_ | |
| | CUAUAG-AAAAUA | |
| 1136 | -4_6-6_internal_ | GAUAUUGUGACAUAUCUCUGCACUCAU |
| | loop-symmetric_ | CACACAAAAGAUGCAAAUCUUGGCCUC |
| | CAGAGA-AGAAGA | GAUCCACAGGGAGGGGGCAUUUUAAU |
| | 26_6-6_internal_ | AUAAGAAGAAACUAAUCAUC |
| | loop-symmetric_ | |
| | CUAUAG-GGCCUC | |
| 1137 | -4_6-6_internal_ | GAUAUUGUGACAUAUCUCUGCACUCAU |
| | loop-symmetric_ | CACACAAAAGAUGCAAAUCUAUAACUG |
| | CAGAGA-ACCACC | GAUCCACAGGGAGGGGGCAUUUUAAC |
| | 0_1-1_mismatch_ | AUAACCACCAACUAAUCAUC |
| | A-C | |
| | 27_6-6_internal_ | |
| | loop-symmetric_ | |
| | UAUAGA-AUAACU | |
| 1138 | -4_6-6_internal_ | GAUAUUGUGACAUAUCUCUGCACUCAU |
| | loop-symmetric_ | CACACAAAAGAUGCAAAUCUCAGCAUG |
| | CAGAGA-CGCCAC | GAUCCACAGGGAGGGGGCAUUUCAAU |
| | 3_1-1_mismatch_ | AUACGCCACAACUAAUCAUC |
| | A-C | |
| | 27_6-6_internal_ | |
| | loop-symmetric_ | |
| | UAUAGA-CAGCAU | |
| 1139 | -4_6-6_internal_ | GAUAUUGUGACAUAUCUCUGCACUCAU |
| | loop-symmetric_ | CACACAAAAGAUGCAAAUCUGACGGCG |
| | CAGAGA-CACAUA | GAUCCACAGGGAGGGGGCAUUCUAAU |
| | 4_1-1_mismatch_ | AUACACAUAAACUAAUCAUC |
| | A-C | |
| | 27_6-6_internal_ | |
| | loop-symmetric_ | |
| | UAUAGA-GACGGC | |
| 1140 | -4_6-6_internal_ | GAUAUUGUGACAUAUCUCUGCACUCAU |
| | loop-symmetric_ | CACACAAAAGAUGCAAAUCUAGCGAUG |
| | CAGAGA-GUAGAC | GAUCCACAGGGAGGGGGCAUCUUAAU |
| | 5_1-1_mismatch_ | AUAGUAGACAACUAAUCAUC |
| | A-C | |
| | 27_6-6_internal_ | |
| | loop-symmetric_ | |
| | UAUAGA-AGCGAU | |
| 1141 | -4_6-6_internal_ | GAUAUUGUGACAUAUCUCUGCACUCAU |
| | loop-symmetric_ | CACACAAAAGAUGCAAAUCUGAGUCCG |
| | CAGAGA-AUGGGU | GAUCCACAGGGAGGGGGCAUUUUAAU |
| | 27_6-6_internal_ | AUAAUGGGUAACUAAUCAUC |
| | loop-symmetric_ | |
| | UAUAGA-GAGUCC | |
| 1142 | -4_6-6_internal_ | GAUAUUGUGACAUAUCUCUGCACUCAU |
| | loop-symmetric_ | CACACAAAAGAUGCAAAUCGCGACGAG |
| | CAGAGA-GGAGUC | GAUCCACAGGGAGGGGGCAUUUUAAC |
| | 0_1-1_mismatch_ | AUAGGAGUCAACUAAUCAUC |
| | A-C | |
| | 28_6-6_internal_ | |
| | loop-symmetric_ | |
| | AUAGAA-GCGACG | |
| 1143 | -4_6-6_internal_ | GAUAUUGUGACAUAUCUCUGCACUCAU |
| | loop-symmetric_ | CACACAAAAGAUGCAAAUCCCAGGGAG |
| | CAGAGA-AGCACC | GAUCCACAGGGAGGGGGCAUUUCAAU |
| | 3_1-1_mismatch_ | AUAAGCACCAACUAAUCAUC |
| | A-C | |
| | 28_6-6_internal_ | |
| | loop-symmetric_ | |
| | AUAGAA-CCAGGG | |
| 1144 | -4_6-6_internal_ | GAUAUUGUGACAUAUCUCUGCACUCAU |
| | loop-symmetric_ | CACACAAAAGAUGCAAAUCAACAAGAG |
| | CAGAGA-AUGACC | GAUCCACAGGGAGGGGGCAUUCUAAU |
| | 4_1-1_mismatch_ | AUAAUGACCAACUAAUCAUC |
| | A-C | |
| | 28_6-6_internal_ | |
| | loop-symmetric_ | |
| | AUAGAA-AACAAG | |
| 1145 | -4_6-6_internal_ | GAUAUUGUGACAUAUCUCUGCACUCAU |
| | loop-symmetric_ | CACACAAAAGAUGCAAAUCGUACCGAG |
| | CAGAGA-GAGUAU | GAUCCACAGGGAGGGGGCAUCUUAAU |
| | 5_1-1_mismatch_ | AUAGAGUAUAACUAAUCAUC |
| | A-C | |
| | 28_6-6_internal_ | |
| | loop-symmetric_ | |
| | AUAGAA-GUACCG | |
| 1146 | -4_6-6_internal_ | GAUAUUGUGACAUAUCUCUGCACUCAU |
| | loop-symmetric_ | CACACAAAAGAUGCAAAUCGGUCAGAG |
| | CAGAGA-GGCCGC | GAUCCACAGGGAGGGGGCAUUUUAAU |
| | 28_6-6_internal_ | AUAGGCCGCAACUAAUCAUC |
| | loop-symmetric_ | |
| | AUAGAA-GGUCAG | |
| 1147 | -4_6-6_internal_ | GAUAUUGUGACAUAUCUCUGCACUCAU |
| | loop-symmetric_ | CACACAAAAGAUGCAAAUGCAUCCUAG |
| | CAGAGA-CGGGUC | GAUCCACAGGGAGGGGGCAUUUUAAC |
| | 0_1-1_mismatch_ | AUACGGGUCAACUAAUCAUC |
| | A-C | |
| | 29_6-6_internal_ | |
| | loop-symmetric_ | |
| | UAGAAG-GCAUCC | |
| 1148 | -4_6-6_internal_ | GAUAUUGUGACAUAUCUCUGCACUCAU |
| | loop-symmetric_ | CACACAAAAGAUGCAAAUAGAUUCUA |
| | CAGAGA-AAAAGU | GGAUCCACAGGGAGGGGGCAUUUCAA |
| | 3_1-1_mismatch_ | UAUAAAAAGUAACUAAUCAUC |
| | A-C | |
| | 29_6-6_internal_ | |
| | loop-symmetric_ | |
| | UAGAAG-AGAUUC | |
| 1149 | -4_6-6_internal_ | GAUAUUGUGACAUAUCUCUGCACUCAU |
| | loop-symmetric_ | CACACAAAAGAUGCAAAUGAGGCCUAG |
| | CAGAGA-CGGAAU | GAUCCACAGGGAGGGGGCAUUCUAAU |
| | 4_1-1_mismatch_ | AUACGGAAUAACUAAUCAUC |
| | A-C | |
| | 29_6-6_internal_ | |
| | loop-symmetric_ | |
| | UAGAAG-GAGGCC | |
| 1150 | -4_6-6_internal_ | GAUAUUGUGACAUAUCUCUGCACUCAU |
| | loop-symmetric_ | CACACAAAAGAUGCAAAUAAAAGUUA |
| | CAGAGA-GGGAGC | GGAUCCACAGGGAGGGGGCAUCUUAA |
| | 5_1-1_mismatch_ | UAUAGGGAGCAACUAAUCAUC |
| | A-C | |
| | 29_6-6_internal_ | |
| | loop-symmetric_ | |
| | UAGAAG-AAAAGU | |
| 1151 | -4_6-6_internal_ | GAUAUUGUGACAUAUCUCUGCACUCAU |
| | loop-symmetric_ | CACACAAAAGAUGCAAAUGACAGUUA |
| | CAGAGA-GACGUC | GGAUCCACAGGGAGGGGGCAUUUUAA |
| | 29_6-6_internal_ | UAUAGACGUCAACUAAUCAUC |
| | loop-symmetric_ | |
| | UAGAAG-GACAGU | |
| 1152 | -4_6-6_internal_ | GAUAUUGUGACAUAUCUCUGCACUCAU |
| | loop-symmetric_ | CACACAAAAGAUGCAAACAACGGAUAG |
| | CAGAGA-AUGGGA | GAUCCACAGGGAGGGGGCAUUUUAAC |
| | 0_1-1_mismatch_ | AUAAUGGGAAACUAAUCAUC |
| | A-C | |
| | 30_6-6_internal_ | |
| | loop-symmetric_ | |
| | AGAAGA-CAACGG | |
| 1153 | -4_6-6_internal_ | GAUAUUGUGACAUAUCUCUGCACUCAU |
| | loop-symmetric_ | CACACAAAAGAUGCAAAAACCGAAUAG |
| | CAGAGA-CCAGGU | GAUCCACAGGGAGGGGGCAUUUCAAU |
| | 3_1-1_mismatch_ | AUACCAGGUAACUAAUCAUC |
| | A-C | |
| | 30_6-6_internal_ | |
| | loop-symmetric_ | |
| | AGAAGA-AACCGA | |
| 1154 | -4_6-6_internal_ | GAUAUUGUGACAUAUCUCUGCACUCAU |
| | loop-symmetric_ | CACACAAAAGAUGCAAAAACGACAUAG |
| | CAGAGA-GAAACC | GAUCCACAGGGAGGGGGCAUUCUAAU |
| | 4_1-1_mismatch_ | AUAGAAACCAACUAAUCAUC |
| | A-C | |
| | 30_6-6_internal_ | |
| | loop-symmetric_ | |
| | AGAAGA-AACGAC | |
| 1155 | -4_6-6_internal_ | GAUAUUGUGACAUAUCUCUGCACUCAU |
| | loop-symmetric_ | CACACAAAAGAUGCAAAAUGCACAUAG |
| | CAGAGA-AUAUAA | GAUCCACAGGGAGGGGGCAUCUUAAU |
| | 5_1-1_mismatch_ | AUAAUAUAAAACUAAUCAUC |
| | A-C | |
| | 30_6-6_internal_ | |
| | loop-symmetric_ | |
| | AGAAGA-AUGCAC | |
| 1156 | -4_6-6_internal_ | GAUAUUGUGACAUAUCUCUGCACUCAU |
| | loop-symmetric_ | CACACAAAAGAUGCAAAGGCCGGAUAG |
| | CAGAGA-AGCAGA | GAUCCACAGGGAGGGGGCAUUUUAAU |
| | 30_6-6_internal_ | AUAAGCAGAAACUAAUCAUC |
| | loop-symmetric_ | |
| | AGAAGA-GGCCGG | |
| 1157 | -4_6-6_internal_ | GAUAUUGUGACAUAUCUCUGCACUCAU |
| | loop-symmetric_ | CACACAAAAGAUGCAACGAAAGUAUA |
| | CAGAGA-GCCGCA | GGAUCCACAGGGAGGGGGCAUUUUAA |
| | 0_1-1_mismatch_ | CAUAGCCGCAAACUAAUCAUC |
| | A-C | |
| | 31_6-6_internal_ | |
| | loop-symmetric_ | |
| | GAAGAU-CGAAAG | |
| 1158 | -4_6-6_internal_ | GAUAUUGUGACAUAUCUCUGCACUCAU |
| | loop-symmetric_ | CACACAAAAGAUGCAAUAAUGGUAUA |
| | CAGAGA-GCACAU | GGAUCCACAGGGAGGGGGCAUUUCAA |
| | 3_1-1_mismatch_ | UAUAGCACAUAACUAAUCAUC |
| | A-C | |
| | 31_6-6_internal_ | |
| | loop-symmetric_ | |
| | GAAGAU-UAAUGG | |
| 1159 | -4_6-6_internal_ | GAUAUUGUGACAUAUCUCUGCACUCAU |
| | loop-symmetric_ | CACACAAAAGAUGCAACACAUAUAUAG |
| | CAGAGA-AGCAAU | GAUCCACAGGGAGGGGGCAUUCUAAU |
| | 4_1-1_mismatch_ | AUAAGCAAUAACUAAUCAUC |
| | A-C | |
| | 31_6-6_internal_ | |
| | loop-symmetric_ | |
| | GAAGAU-CACAUA | |
| 1160 | -4_6-6_internal_ | GAUAUUGUGACAUAUCUCUGCACUCAU |
| | loop-symmetric_ | CACACAAAAGAUGCAAUUGCUAUAUA |
| | CAGAGA-GAAGAC | GGAUCCACAGGGAGGGGGCAUCUUAA |
| | 5_1-1_mismatch_ | UAUAGAAGACAACUAAUCAUC |
| | A-C | |
| | 31_6-6_internal_ | |
| | loop-symmetric_ | |
| | GAAGAU-UUGCUA | |
| 1161 | -4_6-6_internal_ | GAUAUUGUGACAUAUCUCUGCACUCAU |
| | loop-symmetric_ | CACACAAAAGAUGCAACAAUUAUAUA |
| | CAGAGA-GGCGCC | GGAUCCACAGGGAGGGGGCAUUUUAA |
| | 31_6-6_internal_ | UAUAGGCGCCAACUAAUCAUC |
| | loop-symmetric_ | |
| | GAAGAU-CAAUUA | |
| 1162 | -4_6-6_internal_ | GAUAUUGUGACAUAUCUCUGCACUCAU |
| | loop-symmetric_ | CACACAAAAGAUGCAUCGAGCCUAUAG |
| | CAGAGA-AAGGUU | GAUCCACAGGGAGGGGGCAUUUUAAC |
| | 0_1-1_mismatch_ | AUAAAGGUUAACUAAUCAUC |
| | A-C | |
| | 32_6-6_internal_ | |
| | loop-symmetric_ | |
| | AAGAUU-UCGAGC | |
| 1163 | -4_6-6_internal_ | GAUAUUGUGACAUAUCUCUGCACUCAU |
| | loop-symmetric_ | CACACAAAAGAUGCACCCAGCCUAUAG |
| | CAGAGA-AUGAGA | GAUCCACAGGGAGGGGGCAUUUCAAU |
| | 3_1-1_mismatch_ | AUAAUGAGAAACUAAUCAUC |
| | A-C | |
| | 32_6-6_internal_ | |
| | loop-symmetric_ | |
| | AAGAUU-CCCAGC | |
| 1164 | -4_6-6_internal_ | GAUAUUGUGACAUAUCUCUGCACUCAU |
| | loop-symmetric_ | CACACAAAAGAUGCACUCACACUAUAG |
| | CAGAGA-GGUGCA | GAUCCACAGGGAGGGGGCAUUCUAAU |
| | 4_1-1_mismatch_ | AUAGGUGCAAACUAAUCAUC |
| | A-C | |
| | 32_6-6_internal_ | |
| | loop-symmetric_ | |
| | AAGAUU-CUCACA | |
| 1165 | -4_6-6_internal_ | GAUAUUGUGACAUAUCUCUGCACUCAU |
| | loop-symmetric_ | CACACAAAAGAUGCACUGGGCCUAUAG |
| | CAGAGA-GGCACC | GAUCCACAGGGAGGGGGCAUCUUAAU |
| | 5_1-1_mismatch_ | AUAGGCACCAACUAAUCAUC |
| | A-C | |
| | 32_6-6_internal_ | |
| | loop-symmetric_ | |
| | AAGAUU-CUGGGC | |
| 1166 | -4_6-6_internal_ | GAUAUUGUGACAUAUCUCUGCACUCAU |
| | loop-symmetric_ | CACACAAAAGAUGCACCCGGACUAUAG |
| | CAGAGA-AUACAC | GAUCCACAGGGAGGGGGCAUUUUAAU |
| | 32_6-6_internal_ | AUAAUACACAACUAAUCAUC |
| | loop-symmetric_ | |
| | AAGAUU-CCCGGA | |
| 1167 | -4_6-6_internal_ | GAUAUUGUGACAUAUCUCUGCACUCAU |
| | loop-symmetric_ | CACACAAAAGAUGCUCAGGCUCUAUAG |
| | CAGAGA-AAAGCC | GAUCCACAGGGAGGGGGCAUUUUAAC |
| | 0_1-1_mismatch_ | AUAAAAGCCAACUAAUCAUC |
| | A-C | |
| | 33_6-6_internal_ | |
| | loop-symmetric_ | |
| | AGAUUU-UCAGGC | |
| 1168 | -4_6-6_internal_ | GAUAUUGUGACAUAUCUCUGCACUCAU |
| | loop-symmetric_ | CACACAAAAGAUGCCGCGGAUCUAUAG |
| | CAGAGA-GUAGUC | GAUCCACAGGGAGGGGGCAUUUCAAU |
| | 3_1-1_mismatch_ | AUAGUAGUCAACUAAUCAUC |
| | A-C | |
| | 33_6-6_internal_ | |
| | loop-symmetric_ | |
| | AGAUUU-CGCGGA | |
| 1169 | -4_6-6_internal_ | GAUAUUGUGACAUAUCUCUGCACUCAU |
| | loop-symmetric_ | CACACAAAAGAUGCCUGGCGUCUAUAG |
| | CAGAGA-CGAAAU | GAUCCACAGGGAGGGGGCAUUCUAAU |
| | 4_1-1_mismatch_ | AUACGAAAUAACUAAUCAUC |
| | A-C | |
| | 33_6-6_internal_ | |
| | loop-symmetric_ | |
| | AGAUUU-CUGGCG | |
| 1170 | -4_6-6_internal_ | GAUAUUGUGACAUAUCUCUGCACUCAU |
| | loop-symmetric_ | CACACAAAAGAUGCCUGCGGUCUAUAG |
| | CAGAGA-AGGGAA | GAUCCACAGGGAGGGGGCAUCUUAAU |
| | 5_1-1_mismatch_ | AUAAGGGAAAACUAAUCAUC |
| | A-C | |
| | 33_6-6_internal_ | |
| | loop-symmetric_ | |
| | AGAUUU-CUGCGG | |
| 1171 | -4_6-6_internal_ | GAUAUUGUGACAUAUCUCUGCACUCAU |
| | loop-symmetric_ | CACACAAAAGAUGCCCCGAAUCUAUAG |
| | CAGAGA-CCCAAA | GAUCCACAGGGAGGGGGCAUUUUAAU |
| | 33_6-6_internal_ | AUACCCAAAAACUAAUCAUC |
| | loop-symmetric_ | |
| | AGAUUU-CCCGAA | |
| 1172 | -4_6-6_internal_ | GAUAUUGUGACAUAUCUCUGCACUCAU |
| | loop-symmetric_ | CACACAAAAGAUGACGCCGUUCUAUAG |
| | CAGAGA-GAGGAU | GAUCCACAGGGAGGGGGCAUUUUAAC |
| | 0_1-1_mismatch_ | AUAGAGGAUAACUAAUCAUC |
| | A-C | |
| | 34_6-6_internal_ | |
| | loop-symmetric_ | |
| | GAUUUG-ACGCCG | |
| 1173 | -4_6-6_internal_ | GAUAUUGUGACAUAUCUCUGCACUCAU |
| | loop-symmetric_ | CACACAAAAGAUGAAUACGUUCUAUA |
| | CAGAGA-AGCGUU | GGAUCCACAGGGAGGGGGCAUUUCAA |
| | 3_1-1_mismatch_ | UAUAAGCGUUAACUAAUCAUC |
| | A-C | |
| | 34_6-6_internal_ | |
| | loop-symmetric_ | |
| | GAUUUG-AAUACG | |
| 1174 | -4_6-6_internal_ | GAUAUUGUGACAUAUCUCUGCACUCAU |
| | loop-symmetric_ | CACACAAAAGAUGGUUCAGUUCUAUA |
| | CAGAGA-AUGGGA | GGAUCCACAGGGAGGGGGCAUUCUAA |
| | 4_1-1_mismatch_ | UAUAAUGGGAAACUAAUCAUC |
| | A-C | |
| | 34_6-6_internal_ | |
| | loop-symmetric_ | |
| | GAUUUG-GUUCAG | |
| 1175 | -4_6-6_internal_ | GAUAUUGUGACAUAUCUCUGCACUCAU |
| | loop-symmetric_ | CACACAAAAGAUGGUACCAUUCUAUAG |
| | CAGAGA-GGAGCC | GAUCCACAGGGAGGGGGCAUCUUAAU |
| | 5_1-1_mismatch_ | AUAGGAGCCAACUAAUCAUC |
| | A-C | |
| | 34_6-6_internal_ | |
| | loop-symmetric_ | |
| | GAUUUG-GUACCA | |
| 1176 | -4_6-6_internal_ | GAUAUUGUGACAUAUCUCUGCACUCAU |
| | loop-symmetric_ | CACACAAAAGAUGGUUGAGUUCUAUA |
| | CAGAGA-GACGGC | GGAUCCACAGGGAGGGGGCAUUUUAA |
| | 34_6-6_internal_ | UAUAGACGGCAACUAAUCAUC |
| | loop-symmetric_ | |
| | GAUUUG-GUUGAG | |
| 1177 | -4_6-6_internal_ | GAUAUUGUGACAUAUCUCUGCACUCAU |
| | loop-symmetric_ | CACACAAAAGAUAACUCACUUCUAUAG |
| | CAGAGA-GAGUAU | GAUCCACAGGGAGGGGGCAUUUUAAC |
| | 0_1-1_mismatch_ | AUAGAGUAUAACUAAUCAUC |
| | A-C | |
| | 35_6-6_internal_ | |
| | loop-symmetric_ | |
| | AUUUGC-AACUCA | |
| 1178 | -4_6-6_internal_ | GAUAUUGUGACAUAUCUCUGCACUCAU |
| | loop-symmetric_ | CACACAAAAGAUUGACCACUUCUAUAG |
| | CAGAGA-AACCGC | GAUCCACAGGGAGGGGGCAUUUCAAU |
| | 3_1-1_mismatch_ | AUAAACCGCAACUAAUCAUC |
| | A-C | |
| | 35_6-6_internal_ | |
| | loop-symmetric_ | |
| | AUUUGC-UGACCA | |
| 1179 | -4_6-6_internal_ | GAUAUUGUGACAUAUCUCUGCACUCAU |
| | loop-symmetric_ | CACACAAAAGAUCUUACGCUUCUAUAG |
| | CAGAGA-CGCACA | GAUCCACAGGGAGGGGGCAUUCUAAU |
| | 4_1-1_mismatch_ | AUACGCACAAACUAAUCAUC |
| | A-C | |
| | 35_6-6_internal_ | |
| | loop-symmetric_ | |
| | AUUUGC-CUUACG | |
| 1180 | -4_6-6_internal_ | GAUAUUGUGACAUAUCUCUGCACUCAU |
| | loop-symmetric_ | CACACAAAAGAUUUACCACUUCUAUAG |
| | CAGAGA-CGGACA | GAUCCACAGGGAGGGGGCAUCUUAAU |
| | 5_1-1_mismatch_ | AUACGGACAAACUAAUCAUC |
| | A-C | |
| | 35_6-6_internal_ | |
| | loop-symmetric_ | |
| | AUUUGC-UUACCA | |
| 1181 | -4_6-6_internal_ | GAUAUUGUGACAUAUCUCUGCACUCAU |
| | loop-symmetric_ | CACACAAAAGAUCUCCACCUUCUAUAG |
| | CAGAGA-AACACC | GAUCCACAGGGAGGGGGCAUUUUAAU |
| | 35_6-6_internal_ | AUAAACACCAACUAAUCAUC |
| | loop-symmetric_ | |
| | AUUUGC-CUCCAC | |
| 1182 | -4_6-6_internal_ | GAUAUUGUGACAUAUCUCUGCACUCAU |
| | loop-symmetric_ | CACACAAAAGAGAGGCUUCUUCUAUAG |
| | CAGAGA-CAGGAC | GAUCCACAGGGAGGGGGCAUUUUAAC |
| | 0_1-1_mismatch_ | AUACAGGACAACUAAUCAUC |
| | A-C | |
| | 36_6-6_internal_ | |
| | loop-symmetric_ | |
| | UUUGCA-GAGGCU | |
| 1183 | -4_6-6_internal_ | GAUAUUGUGACAUAUCUCUGCACUCAU |
| | loop-symmetric_ | CACACAAAAGAGAAUCCUCUUCUAUAG |
| | CAGAGA-AUGGCC | GAUCCACAGGGAGGGGGCAUUUCAAU |
| | 3_1-1_mismatch_ | AUAAUGGCCAACUAAUCAUC |
| | A-C | |
| | 36_6-6_internal_ | |
| | loop-symmetric_ | |
| | UUUGCA-GAAUCC | |
| 1184 | -4_6-6_internal_ | GAUAUUGUGACAUAUCUCUGCACUCAU |
| | loop-symmetric_ | CACACAAAAGAAAGUCCUCUUCUAUAG |
| | CAGAGA-GCAGCA | GAUCCACAGGGAGGGGGCAUUCUAAU |
| | 4_1-1_mismatch_ | AUAGCAGCAAACUAAUCAUC |
| | A-C | |
| | 36_6-6_internal_ | |
| | loop-symmetric_ | |
| | UUUGCA-AAGUCC | |
| 1185 | -4_6-6_internal_ | GAUAUUGUGACAUAUCUCUGCACUCAU |
| | loop-symmetric_ | CACACAAAAGAGACCUCUCUUCUAUAG |
| | CAGAGA-AAGGUC | GAUCCACAGGGAGGGGGCAUCUUAAU |
| | 5_1-1_mismatch_ | AUAAAGGUCAACUAAUCAUC |
| | A-C | |
| | 36_6-6_internal_ | |
| | loop-symmetric_ | |
| | UUUGCA-GACCUC | |
| 1186 | -4_6-6_internal_ | GAUAUUGUGACAUAUCUCUGCACUCAU |
| | loop-symmetric_ | CACACAAAAGCAAUACAUCUUCUAUAG |
| | CAGAGA-GCGGUC | GAUCCACAGGGAGGGGGCAUUUUAAC |
| | 0_1-1_mismatch_ | AUAGCGGUCAACUAAUCAUC |
| | A-C | |
| | 37_6-6_internal_ | |
| | loop-symmetric_ | |
| | UUGCAU-CAAUAC | |
| 1187 | -4_6-6_internal_ | GAUAUUGUGACAUAUCUCUGCACUCAU |
| | loop-symmetric_ | CACACAAAAGCAUAUCAUCUUCUAUAG |
| | CAGAGA-AACAGA | GAUCCACAGGGAGGGGGCAUUUCAAU |
| | 3_1-1_mismatch_ | AUAAACAGAAACUAAUCAUC |
| | A-C | |
| | 37_6-6_internal_ | |
| | loop-symmetric_ | |
| | UUGCAU-CAUAUC | |
| 1188 | -4_6-6_internal_ | GAUAUUGUGACAUAUCUCUGCACUCAU |
| | loop-symmetric_ | CACACAAAAGCUAGAUAUCUUCUAUAG |
| | CAGAGA-CACCCA | GAUCCACAGGGAGGGGGCAUUCUAAU |
| | 4_1-1_mismatch_ | AUACACCCAAACUAAUCAUC |
| | A-C | |
| | 37_6-6_internal_ | |
| | loop-symmetric_ | |
| | UUGCAU-CUAGAU | |
| 1189 | -4_6-6_internal_ | GAUAUUGUGACAUAUCUCUGCACUCAU |
| | loop-symmetric_ | CACACAAAAGUCAGUUAUCUUCUAUAG |
| | CAGAGA-AUAGAU | GAUCCACAGGGAGGGGGCAUCUUAAU |
| | 5_1-1_mismatch_ | AUAAUAGAUAACUAAUCAUC |
| | A-C | |
| | 37_6-6_internal_ | |
| | loop-symmetric_ | |
| | UUGCAU-UCAGUU | |
| 1190 | -4_6-6_internal_ | GAUAUUGUGACAUAUCUCUGCACUCAU |
| | loop-symmetric_ | CACACAAAAGCUAGGUAUCUUCUAUAG |
| | CAGAGA-CGAGCC | GAUCCACAGGGAGGGGGCAUUUUAAU |
| | 37_6-6_internal_ | AUACGAGCCAACUAAUCAUC |
| | loop-symmetric_ | |
| | UUGCAU-CUAGGU | |
| 1191 | -4_6-6_internal_ | GAUAUUGUGACAUAUCUCUGCACUCAU |
| | loop-symmetric_ | CACACAAAAUCCAAUAAUCUUCUAUAG |
| | CAGAGA-GUAUUC | GAUCCACAGGGAGGGGGCAUUUUAAC |
| | 0_1-1_mismatch_ | AUAGUAUUCAACUAAUCAUC |
| | A-C | |
| | 38_6-6_internal_ | |
| | loop-symmetric_ | |
| | UGCAUC-UCCAAU | |
| 1192 | -4_6-6_internal_ | GAUAUUGUGACAUAUCUCUGCACUCAU |
| | loop-symmetric_ | CACACAAAACGGACCAAUCUUCUAUAG |
| | CAGAGA-CCAAAC | GAUCCACAGGGAGGGGGCAUUUCAAU |
| | 3_1-1_mismatch_ | AUACCAAACAACUAAUCAUC |
| | A-C | |
| | 38_6-6_internal_ | |
| | loop-symmetric_ | |
| | UGCAUC-CGGACC | |
| 1193 | -4_6-6_internal_ | GAUAUUGUGACAUAUCUCUGCACUCAU |
| | loop-symmetric_ | CACACAAAACCCCCUAAUCUUCUAUAG |
| | CAGAGA-CGGGCA | GAUCCACAGGGAGGGGGCAUUCUAAU |
| | 4_1-1_mismatch_ | AUACGGGCAAACUAAUCAUC |
| | A-C | |
| | 38_6-6_internal_ | |
| | loop-symmetric_ | |
| | UGCAUC-CCCCCU | |
| 1194 | -4_6-6_internal_ | GAUAUUGUGACAUAUCUCUGCACUCAU |
| | loop-symmetric_ | CACACAAAACCGUUUAAUCUUCUAUAG |
| | CAGAGA-CGGUAU | |
| | 5_1-1_mismatch_ | GAUCCACAGGGAGGGGGCAUCUUAAU |
| | A-C | AUACGGUAUAACUAAUCAUC |
| | 38_6-6_internal_ | |
| | loop-symmetric_ | |
| | UGCAUC-CCGUUU | |
| 1195 | -4_6-6_internal_ | GAUAUUGUGACAUAUCUCUGCACUCAU |
| | loop-symmetric_ | CACACAAAACAACCUAAUCUUCUAUAG |
| | CAGAGA-AUAGAC | GAUCCACAGGGAGGGGGCAUUUUAAU |
| | 38_6-6_internal_ | AUAAUAGACAACUAAUCAUC |
| | loop-symmetric_ | |
| | UGCAUC-CAACCU | |
| 1196 | -4_6-6_internal_ | GAUAUUGUGACAUAUCUCUGCACUCAU |
| | loop-symmetric_ | CACACAAACCCCCGAAAUCUUCUAUAG |
| | CAGAGA-AGCACA | GAUCCACAGGGAGGGGGCAUUUUAAC |
| | 0_1-1_mismatch_ | AUAAGCACAAACUAAUCAUC |
| | A-C | |
| | 39_6-6_internal_ | |
| | loop-symmetric_ | |
| | GCAUCU-CCCCCG | |
| 1197 | -4_6-6_internal_ | GAUAUUGUGACAUAUCUCUGCACUCAU |
| | loop-symmetric_ | CACACAAACCGCAGAAAUCUUCUAUAG |
| | CAGAGA-AAAGUA | GAUCCACAGGGAGGGGGCAUUUCAAU |
| | 3_1-1_mismatch_ | AUAAAAGUAAACUAAUCAUC |
| | A-C | |
| | 39_6-6_internal_ | |
| | loop-symmetric_ | |
| | GCAUCU-CCGCAG | |
| 1198 | -4_6-6_internal_ | GAUAUUGUGACAUAUCUCUGCACUCAU |
| | loop-symmetric_ | CACACAAACCCAAAAAAUCUUCUAUAG |
| | CAGAGA-GCCGCA | GAUCCACAGGGAGGGGGCAUUCUAAU |
| | 4_1-1_mismatch_ | AUAGCCGCAAACUAAUCAUC |
| | A-C | |
| | 39_6-6_internal_ | |
| | loop-symmetric_ | |
| | GCAUCU-CCCAAA | |
| 1199 | -4_6-6_internal_ | GAUAUUGUGACAUAUCUCUGCACUCAU |
| | loop-symmetric_ | CACACAAACCACAAAAAUCUUCUAUAG |
| | CAGAGA-GAACGC | GAUCCACAGGGAGGGGGCAUCUUAAU |
| | 5_1-1_mismatch_ | AUAGAACGCAACUAAUCAUC |
| | A-C | |
| | 39_6-6_internal_ | |
| | loop-symmetric_ | |
| | GCAUCU-CCACAA | |
| 1200 | -4_6-6_internal_ | GAUAUUGUGACAUAUCUCUGCACUCAU |
| | loop-symmetric_ | CACACAAAUUUGAGAAAUCUUCUAUA |
| | CAGAGA-GCAACC | GGAUCCACAGGGAGGGGGCAUUUUAA |
| | 39_6-6_internal_ | UAUAGCAACCAACUAAUCAUC |
| | loop-symmetric_ | |
| | GCAUCU-UUUGAG | |
| 1201 | -4_6-6_internal_ | GAUAUUGUGACAUAUCUCUGCACUCAU |
| | loop-symmetric_ | CACACAACCUAAUCAAAUCUUCUAUAG |
| | CAGAGA-GAAGUU | GAUCCACAGGGAGGGGGCAUUUUAAC |
| | 0_1-1_mismatch_ | AUAGAAGUUAACUAAUCAUC |
| | A-C | |
| | 40_6-6_internal_ | |
| | loop-symmetric_ | |
| | CAUCUU-CCUAAU | |
| 1202 | -4_6-6_internal_ | GAUAUUGUGACAUAUCUCUGCACUCAU |
| | loop-symmetric_ | CACACAACACGCUCAAAUCUUCUAUAG |
| | CAGAGA-AGAGAA | GAUCCACAGGGAGGGGGCAUUUCAAU |
| | 3_1-1_mismatch_ | AUAAGAGAAAACUAAUCAUC |
| | A-C | |
| | 40_6-6_internal_ | |
| | loop-symmetric_ | |
| | CAUCUU-CACGCU | |
| 1203 | -4_6-6_internal_ | GAUAUUGUGACAUAUCUCUGCACUCAU |
| | loop-symmetric_ | CACACAACACGCCCAAAUCUUCUAUAG |
| | CAGAGA-CCAGAC | GAUCCACAGGGAGGGGGCAUUCUAAU |
| | 4_1-1_mismatch_ | AUACCAGACAACUAAUCAUC |
| | A-C | |
| | 40_6-6_internal_ | |
| | loop-symmetric_ | |
| | CAUCUU-CACGCC | |
| 1204 | -4_6-6_internal_ | GAUAUUGUGACAUAUCUCUGCACUCAU |
| | loop-symmetric_ | CACACAACAUAGCCAAAUCUUCUAUAG |
| | CAGAGA-CGGUAA | GAUCCACAGGGAGGGGGCAUCUUAAU |
| | 5_1-1_mismatch_ | AUACGGUAAAACUAAUCAUC |
| | A-C | |
| | 40_6-6_internal_ | |
| | loop-symmetric_ | |
| | CAUCUU-CAUAGC | |
| 1205 | -4_6-6_internal_ | GAUAUUGUGACAUAUCUCUGCACUCAU |
| | loop-symmetric_ | CACACAACUACUCCAAAUCUUCUAUAG |
| | CAGAGA-GUGGCC | GAUCCACAGGGAGGGGGCAUUUUAAU |
| | 40_6-6_internal_ | AUAGUGGCCAACUAAUCAUC |
| | loop-symmetric_ | |
| | CAUCUU-CUACUC | |
| 1206 | -4_6-6_internal_ | GAUAUUGUGACAUAUCUCUGCACUCAU |
| | loop-symmetric_ | CACACACCAUUCGCAAAUCUUCUAUAG |
| | CAGAGA-GGGAGU | GAUCCACAGGGAGGGGGCAUUUUAAC |
| | 0_1-1_mismatch_ | AUAGGGAGUAACUAAUCAUC |
| | A-C | |
| | 41_6-6_internal_ | |
| | loop-symmetric_ | |
| | AUCUUU-CCAUUC | |
| 1207 | -4_6-6_internal_ | GAUAUUGUGACAUAUCUCUGCACUCAU |
| | loop-symmetric_ | CACACAUGUACGGCAAAUCUUCUAUAG |
| | CAGAGA-CAGGGC | GAUCCACAGGGAGGGGGCAUUUCAAU |
| | 3_1-1_mismatch_ | AUACAGGGCAACUAAUCAUC |
| | A-C | |
| | 41_6-6_internal_ | |
| | loop-symmetric_ | |
| | AUCUUU-UGUACG | |
| 1208 | -4_6-6_internal_ | GAUAUUGUGACAUAUCUCUGCACUCAU |
| | loop-symmetric_ | CACACAUUAAUGGCAAAUCUUCUAUAG |
| | CAGAGA-GAGACC | GAUCCACAGGGAGGGGGCAUUCUAAU |
| | 4_1-1_mismatch_ | AUAGAGACCAACUAAUCAUC |
| | A-C | |
| | 41_6-6_internal_ | |
| | loop-symmetric_ | |
| | AUCUUU-UUAAUG | |
| 1209 | -4_6-6_internal_ | GAUAUUGUGACAUAUCUCUGCACUCAU |
| | loop-symmetric_ | CACACACCAUAGGCAAAUCUUCUAUAG |
| | CAGAGA-GGAGAC | GAUCCACAGGGAGGGGGCAUCUUAAU |
| | 5_1-1_mismatch_ | AUAGGAGACAACUAAUCAUC |
| | A-C | |
| | 41_6-6_internal_ | |
| | loop-symmetric_ | |
| | AUCUUU-CCAUAG | |
| 1210 | -4_6-6_internal_ | GAUAUUGUGACAUAUCUCUGCACUCAU |
| | loop-symmetric_ | CACACACUGCCAGCAAAUCUUCUAUAG |
| | CAGAGA-CAACGC | GAUCCACAGGGAGGGGGCAUUUUAAU |
| | 41_6-6_internal_ | AUACAACGCAACUAAUCAUC |
| | loop-symmetric_ | |
| | AUCUUU-CUGCCA | |
| 1211 | -4_6-6_internal_ | GAUAUUGUGACAUAUCUCUGCACUCAU |
| | loop-symmetric_ | CACACCCCCUCUGCAAAUCUUCUAUAG |
| | CAGAGA-ACAGGC | GAUCCACAGGGAGGGGGCAUUUUAAC |
| | 0_1-1_mismatch_ | AUAACAGGCAACUAAUCAUC |
| | A-C | |
| | 42_6-6_internal_ | |
| | loop-symmetric_ | |
| | UCUUUU-CCCCUC | |
| 1212 | -4_6-6_internal_ | GAUAUUGUGACAUAUCUCUGCACUCAU |
| | loop-symmetric_ | CACACUCGUCCUGCAAAUCUUCUAUAG |
| | CAGAGA-GACAUU | GAUCCACAGGGAGGGGGCAUUUCAAU |
| | 3_1-1_mismatch_ | AUAGACAUUAACUAAUCAUC |
| | A-C | |
| | 42_6-6_internal_ | |
| | loop-symmetric_ | |
| | UCUUUU-UCGUCC | |
| 1213 | -4_6-6_internal_ | GAUAUUGUGACAUAUCUCUGCACUCAU |
| | loop-symmetric_ | CACACCUACACUGCAAAUCUUCUAUAG |
| | CAGAGA-GGGAGU | GAUCCACAGGGAGGGGGCAUUCUAAU |
| | 4_1-1_mismatch_ | AUAGGGAGUAACUAAUCAUC |
| | A-C | |
| | 42_6-6_internal_ | |
| | loop-symmetric_ | |
| | UCUUUU-CUACAC | |
| 1214 | -4_6-6_internal_ | GAUAUUGUGACAUAUCUCUGCACUCAU |
| | loop-symmetric_ | CACACUCUUACUGCAAAUCUUCUAUAG |
| | CAGAGA-GCCACA | GAUCCACAGGGAGGGGGCAUCUUAAU |
| | 5_1-1_mismatch_ | AUAGCCACAAACUAAUCAUC |
| | A-C | |
| | 42_6-6_internal_ | |
| | loop-symmetric_ | |
| | UCUUUU-UCUUAC | |
| 1215 | -4_6-6_internal_ | GAUAUUGUGACAUAUCUCUGCACUCAU |
| | loop-symmetric_ | CACACCUCUCUUGCAAAUCUUCUAUAG |
| | CAGAGA-GUAGCA | GAUCCACAGGGAGGGGGCAUUUUAAU |
| | 42_6-6_internal_ | AUAGUAGCAAACUAAUCAUC |
| | loop-symmetric_ | |
| | UCUUUU-CUCUCU | |
| 1216 | -4_6-6_internal_ | GAUAUUGUGACAUAUCUCUGCACUCAU |
| | loop-symmetric_ | CACAGUAACCAUGCAAAUCUUCUAUAG |
| | CAGAGA-GUAGGU | GAUCCACAGGGAGGGGGCAUUUUAAC |
| | 0_1-1_mismatch_ | AUAGUAGGUAACUAAUCAUC |
| | A-C | |
| | 43_6-6_internal_ | |
| | loop-symmetric_ | |
| | CUUUUG-GUAACC | |
| 1217 | -4_6-6_internal_ | GAUAUUGUGACAUAUCUCUGCACUCAU |
| | loop-symmetric_ | CACAAACUUAAUGCAAAUCUUCUAUAG |
| | CAGAGA-GUGGAA | GAUCCACAGGGAGGGGGCAUUUCAAU |
| | 3_1-1_mismatch_ | AUAGUGGAAAACUAAUCAUC |
| | A-C | |
| | 43_6-6_internal_ | |
| | loop-symmetric_ | |
| | CUUUUG-AACUUA | |
| 1218 | -4_6-6_internal_ | GAUAUUGUGACAUAUCUCUGCACUCAU |
| | loop-symmetric_ | CACAACUCAUAUGCAAAUCUUCUAUAG |
| | CAGAGA-AGGACC | GAUCCACAGGGAGGGGGCAUUCUAAU |
| | 4_1-1_mismatch_ | AUAAGGACCAACUAAUCAUC |
| | A-C | |
| | 43_6-6_internal_ | |
| | loop-symmetric_ | |
| | CUUUUG-ACUCAU | |
| 1219 | -4_6-6_internal_ | GAUAUUGUGACAUAUCUCUGCACUCAU |
| | loop-symmetric_ | CACAGCUAUCAUGCAAAUCUUCUAUAG |
| | CAGAGA-GAAGGA | GAUCCACAGGGAGGGGGCAUCUUAAU |
| | 5_1-1_mismatch_ | AUAGAAGGAAACUAAUCAUC |
| | A-C | |
| | 43_6-6_internal_ | |
| | loop-symmetric_ | |
| | CUUUUG-GCUAUC | |
| 1220 | -4_6-6_internal_ | GAUAUUGUGACAUAUCUCUGCACUCAU |
| | loop-symmetric_ | CACAAGUGCUAUGCAAAUCUUCUAUAG |
| | CAGAGA-GGGAGC | GAUCCACAGGGAGGGGGCAUUUUAAU |
| | 43_6-6_internal_ | AUAGGGAGCAACUAAUCAUC |
| | loop-symmetric_ | |
| | CUUUUG-AGUGCU | |
| 1221 | -4_6-6_internal_ | GAUAUUGUGACAUAUCUCUGCACUCAU |
| | loop-symmetric_ | CACUUCUUCGAUGCAAAUCUUCUAUAG |
| | CAGAGA-AACACA | GAUCCACAGGGAGGGGGCAUUUUAAC |
| | 0_1-1_mismatch_ | AUAAACACAAACUAAUCAUC |
| | A-C | |
| | 44_6-6_internal_ | |
| | loop-symmetric_ | |
| | UUUUGU-UUCUUC | |
| 1222 | -4_6-6_internal_ | GAUAUUGUGACAUAUCUCUGCACUCAU |
| | loop-symmetric_ | CACUUGGCCGAUGCAAAUCUUCUAUAG |
| | CAGAGA-GCCCAU | GAUCCACAGGGAGGGGGCAUUUCAAU |
| | 3_1-1_mismatch_ | AUAGCCCAUAACUAAUCAUC |
| | A-C | |
| | 44_6-6_internal_ | |
| | loop-symmetric_ | |
| | UUUUGU-UUGGCC | |
| 1223 | -4_6-6_internal_ | GAUAUUGUGACAUAUCUCUGCACUCAU |
| | loop-symmetric_ | CACUGGUCCGAUGCAAAUCUUCUAUAG |
| | CAGAGA-GCACAU | GAUCCACAGGGAGGGGGCAUUCUAAU |
| | 4_1-1_mismatch_ | AUAGCACAUAACUAAUCAUC |
| | A-C | |
| | 44_6-6_internal_ | |
| | loop-symmetric_ | |
| | UUUUGU-UGGUCC | |
| 1224 | -4_6-6_internal_ | GAUAUUGUGACAUAUCUCUGCACUCAU |
| | loop-symmetric_ | CACCUUUAUGAUGCAAAUCUUCUAUAG |
| | CAGAGA-CAGAUA | GAUCCACAGGGAGGGGGCAUCUUAAU |
| | 5_1-1_mismatch_ | AUACAGAUAAACUAAUCAUC |
| | A-C | |
| | 44_6-6_internal_ | |
| | loop-symmetric_ | |
| | UUUUGU-CUUUAU | |
| 1225 | -4_6-6_internal_ | GAUAUUGUGACAUAUCUCUGCACUCAU |
| | loop-symmetric_ | CACCUUGUUGAUGCAAAUCUUCUAUAG |
| | CAGAGA-CCCAAA | GAUCCACAGGGAGGGGGCAUUUUAAU |
| | 44_6-6_internal_ | AUACCCAAAAACUAAUCAUC |
| | loop-symmetric_ | |
| | UUUUGU-CUUGUU | |
| 1226 | -3_6-6_internal_ | GAUAUUGUGACAUAUCUCUGCACUCAU |
| | loop-symmetric_ | CACACAAAAGAUGCAAAUCUUCUAUAG |
| | AGAGAU-UGGCGC | GAUCCACAGGGAGGGGGCAUUUUAAC |
| | 0_1-1_mismatch_ | AUUGGCGCGAACUAAUCAUC |
| | A-C | |
| 1227 | -3_6-6_internal_ | GAUAUUGUGACAUAUCUCUGCACUCAU |
| | loop-symmetric_ | CACACAAAAGAUGCAAAUCUUCUAUAG |
| | AGAGAU-UGUACC | GAUCCACAGGGAGGGGGCAUUUCAAU |
| | 3_1-1_mismatch_ | AUUGUACCGAACUAAUCAUC |
| | A-C | |
| 1228 | -3_6-6_internal_ | GAUAUUGUGACAUAUCUCUGCACUCAU |
| | loop-symmetric_ | CACACAAAAGAUGCAAAUCUUCUAUAG |
| | AGAGAU-UCAGAG | GAUCCACAGGGAGGGGGCAUUCUAAU |
| | 4_1-1_mismatch_ | AUUCAGAGGAACUAAUCAUC |
| | A-C | |
| 1229 | -3_6-6_internal_ | GAUAUUGUGACAUAUCUCUGCACUCAU |
| | loop-symmetric_ | CACACAAAAGAUGCAAAUCUUCUAUAG |
| | AGAGAU-CGGCAA | GAUCCACAGGGAGGGGGCAUCUUAAU |
| | 5_1-1_mismatch_ | AUCGGCAAGAACUAAUCAUC |
| | A-C | |
| 1230 | -3_6-6_internal_ | GAUAUUGUGACAUAUCUCUGCACUCAU |
| | loop-symmetric_ | CACACAAAAGAUGCAAAUCUUCUAUAG |
| | AGAGAU-UGGCCC | GAUCCACAGGGAGGGGGCAUUUUAAU |
| | | AUUGGCCCGAACUAAUCAUC |
| 1231 | -3_6-6_internal_ | GAUAUUGUGACAUAUCUCUGCACUCAU |
| | loop-symmetric_ | CACACAAAAGAUGCAAAUCUUCUAAGC |
| | AGAGAU-UGGGGG | CUACCACAGGGAGGGGGCAUUUUAACA |
| | 0_1-1_mismatch_ | UUGGGGGGAACUAAUCAUC |
| | A-C | |
| | 23_6-6_internal_ | |
| | loop-symmetric_ | |
| | AUCCUA-AGCCUA | |
| 1232 | -3_6-6_internal_ | GAUAUUGUGACAUAUCUCUGCACUCAU |
| | loop-symmetric_ | CACACAAAAGAUGCAAAUCUUCUAACU |
| | AGAGAU-CAGAAG | CACCCACAGGGAGGGGGCAUUUCAAUA |
| | 3_1-1_mismatch_ | UCAGAAGGAACUAAUCAUC |
| | A-C | |
| | 23_6-6_internal_ | |
| | loop-symmetric_ | |
| | AUCCUA-ACUCAC | |
| 1233 | -3_6-6_internal_ | GAUAUUGUGACAUAUCUCUGCACUCAU |
| | loop-symmetric_ | CACACAAAAGAUGCAAAUCUUCUACCC |
| | AGAGAU-UCAUAC | ACACCACAGGGAGGGGGCAUUCUAAUA |
| | 4_1-1_mismatch_ | UUCAUACGAACUAAUCAUC |
| | A-C | |
| | 23_6-6_internal_ | |
| | loop-symmetric_ | |
| | AUCCUA-CCCACA | |
| 1234 | -3_6-6_internal_ | GAUAUUGUGACAUAUCUCUGCACUCAU |
| | loop-symmetric_ | CACACAAAAGAUGCAAAUCUUCUAGCC |
| | AGAGAU-UCACCC | GCGCCACAGGGAGGGGGCAUCUUAAUA |
| | 5_1-1_mismatch_ | UUCACCCGAACUAAUCAUC |
| | A-C | |
| | 23_6-6_internal_ | |
| | loop-symmetric_ | |
| | AUCCUA-GCCGCG | |
| 1235 | -3_6-6_internal_ | GAUAUUGUGACAUAUCUCUGCACUCAU |
| | loop-symmetric_ | CACACAAAAGAUGCAAAUCUUCUAGAA |
| | AGAGAU-UGUGGC | AUGCCACAGGGAGGGGGCAUUUUAAU |
| | 23_6-6_internal_ | AUUGUGGCGAACUAAUCAUC |
| | loop-symmetric_ | |
| | AUCCUA-GAAAUG | |
| 1236 | -3_6-6_internal_ | GAUAUUGUGACAUAUCUCUGCACUCAU |
| | loop-symmetric_ | CACACAAAAGAUGCAAAUCUUCUCCUU |
| | AGAGAU-UAGAAA | CUUCCACAGGGAGGGGGCAUUUUAACA |
| | 0_1-1_mismatch_ | UUAGAAAGAACUAAUCAUC |
| | A-C | |
| | 24_6-6_internal_ | |
| | loop-symmetric_ | |
| | UCCUAU-CCUUCU | |
| 1237 | -3_6-6_internal_ | GAUAUUGUGACAUAUCUCUGCACUCAU |
| | loop-symmetric_ | CACACAAAAGAUGCAAAUCUUCUUAUA |
| | AGAGAU-UACCAG | CCUCCACAGGGAGGGGGCAUUUCAAUA |
| | 3_1-1_mismatch_ | UUACCAGGAACUAAUCAUC |
| | A-C | |
| | 24_6-6_internal_ | |
| | loop-symmetric_ | |
| | UCCUAU-UAUACC | |
| 1238 | -3_6-6_internal_ | GAUAUUGUGACAUAUCUCUGCACUCAU |
| | loop-symmetric_ | CACACAAAAGAUGCAAAUCUUCUUUUA |
| | AGAGAU-CAUGGA | UUUCCACAGGGAGGGGGCAUUCUAAU |
| | 4_1-1_mismatch_ | AUCAUGGAGAACUAAUCAUC |
| | A-C | |
| | 24_6-6_internal_ | |
| | loop-symmetric_ | |
| | UCCUAU-UUUAUU | |
| 1239 | -3_6-6_internal_ | GAUAUUGUGACAUAUCUCUGCACUCAU |
| | loop-symmetric_ | CACACAAAAGAUGCAAAUCUUCUUGCA |
| | AGAGAU-UUGGGG | AUUCCACAGGGAGGGGGCAUCUUAAU |
| | 5_1-1_mismatch_ | AUUUGGGGGAACUAAUCAUC |
| | A-C | |
| | 24_6-6_internal_ | |
| | loop-symmetric_ | |
| | UCCUAU-UGCAAU | |
| 1240 | -3_6-6_internal_ | GAUAUUGUGACAUAUCUCUGCACUCAU |
| | loop-symmetric_ | CACACAAAAGAUGCAAAUCUUCCGAGU |
| | AGAGAU-UGAGGG | CAUCCACAGGGAGGGGGCAUUUUAACA |
| | 0_1-1_mismatch_ | UUGAGGGGAACUAAUCAUC |
| | A-C | |
| | 25_6-6_internal_ | |
| | loop-symmetric_ | |
| | CCUAUA-CGAGUC | |
| 1241 | -3_6-6_internal_ | GAUAUUGUGACAUAUCUCUGCACUCAU |
| | loop-symmetric_ | CACACAAAAGAUGCAAAUCUUCCUCGU |
| | AGAGAU-CGGAAG | UAUCCACAGGGAGGGGGCAUUUCAAU |
| | 3_1-1_mismatch_ | AUCGGAAGGAACUAAUCAUC |
| | A-C | |
| | 25_6-6_internal_ | |
| | loop-symmetric_ | |
| | CCUAUA-CUCGUU | |
| 1242 | -3_6-6_internal_ | GAUAUUGUGACAUAUCUCUGCACUCAU |
| | loop-symmetric_ | CACACAAAAGAUGCAAAUCUUCACAAA |
| | AGAGAU-UUGGAA | UAUCCACAGGGAGGGGGCAUUCUAAU |
| | 4_1-1_mismatch_ | AUUUGGAAGAACUAAUCAUC |
| | A-C | |
| | 25_6-6_internal_ | |
| | loop-symmetric_ | |
| | CCUAUA-ACAAAU | |
| 1243 | -3_6-6_internal_ | GAUAUUGUGACAUAUCUCUGCACUCAU |
| | loop-symmetric_ | CACACAAAAGAUGCAAAUCUUCAGAAU |
| | AGAGAU-CCUACG | AAUCCACAGGGAGGGGGCAUCUUAAU |
| | 5_1-1_mismatch_ | AUCCUACGGAACUAAUCAUC |
| | A-C | |
| | 25_6-6_internal_ | |
| | loop-symmetric_ | |
| | CCUAUA-AGAAUA | |
| 1244 | -3_6-6_internal_ | GAUAUUGUGACAUAUCUCUGCACUCAU |
| | loop-symmetric_ | CACACAAAAGAUGCAAAUCUUCACAAC |
| | AGAGAU-UAAGUA | UAUCCACAGGGAGGGGGCAUUUUAAU |
| | 25_6-6_internal_ | AUUAAGUAGAACUAAUCAUC |
| | loop-symmetric_ | |
| | CCUAUA-ACAACU | |
| 1245 | -3_6-6_internal_ | GAUAUUGUGACAUAUCUCUGCACUCAU |
| | loop-symmetric_ | CACACAAAAGAUGCAAAUCUUAACCAC |
| | AGAGAU-CAUAUA | GAUCCACAGGGAGGGGGCAUUUCAAU |
| | 3_1-1_mismatch_ | AUCAUAUAGAACUAAUCAUC |
| | A-C | |
| | 26_6-6_internal_ | |
| | loop-symmetric_ | |
| | CUAUAG-AACCAC | |
| 1246 | -3_6-6_internal_ | GAUAUUGUGACAUAUCUCUGCACUCAU |
| | loop-symmetric_ | CACACAAAAGAUGCAAAUCUUAAGGCA |
| | AGAGAU-UAGUAC | GAUCCACAGGGAGGGGGCAUUCUAAU |
| | 4_1-1_mismatch_ | AUUAGUACGAACUAAUCAUC |
| | A-C | |
| | 26_6-6_internal_ | |
| | loop-symmetric_ | |
| | CUAUAG-AAGGCA | |
| 1247 | -3_6-6_internal_ | GAUAUUGUGACAUAUCUCUGCACUCAU |
| | loop-symmetric_ | CACACAAAAGAUGCAAAUCUUGCCCGA |
| | AGAGAU-UAAAGG | GAUCCACAGGGAGGGGGCAUCUUAAU |
| | 5_1-1_mismatch_ | AUUAAAGGGAACUAAUCAUC |
| | A-C | |
| | 26_6-6_internal_ | |
| | loop-symmetric_ | |
| | CUAUAG-GCCCGA | |
| 1248 | -3_6-6_internal_ | GAUAUUGUGACAUAUCUCUGCACUCAU |
| | loop-symmetric_ | CACACAAAAGAUGCAAAUCUUAUCUUA |
| | AGAGAU-UCCAUA | GAUCCACAGGGAGGGGGCAUUUUAAU |
| | 26_6-6_internal_ | AUUCCAUAGAACUAAUCAUC |
| | loop-symmetric_ | |
| | CUAUAG-AUCUUA | |
| 1249 | -3_6-6_internal_ | GAUAUUGUGACAUAUCUCUGCACUCAU |
| | loop-symmetric_ | CACACAAAAGAUGCAAAUCUCCCCCCG |
| | AGAGAU-UAAAUG | GAUCCACAGGGAGGGGGCAUUUUAAC |
| | 0_1-1_mismatch_ | AUUAAAUGGAACUAAUCAUC |
| | A-C | |
| | 27_6-6_internal_ | |
| | loop-symmetric_ | |
| | UAUAGA-CCCCCC | |
| 1250 | -3_6-6_internal_ | GAUAUUGUGACAUAUCUCUGCACUCAU |
| | loop-symmetric_ | CACACAAAAGAUGCAAAUCUGGGACUG |
| | AGAGAU-UUGGGC | GAUCCACAGGGAGGGGGCAUUUCAAU |
| | 3_1-1_mismatch_ | AUUUGGGCGAACUAAUCAUC |
| | A-C | |
| | 27_6-6_internal_ | |
| | loop-symmetric_ | |
| | UAUAGA-GGGACU | |
| 1251 | -3_6-6_internal_ | GAUAUUGUGACAUAUCUCUGCACUCAU |
| | loop-symmetric_ | CACACAAAAGAUGCAAAUCUGUAUUU |
| | AGAGAU-UUAAGA | GGAUCCACAGGGAGGGGGCAUUCUAA |
| | 4_1-1_mismatch_ | UAUUUAAGAGAACUAAUCAUC |
| | A-C | |
| | 27_6-6_internal_ | |
| | loop-symmetric_ | |
| | UAUAGA-GUAUUU | |
| 1252 | -3_6-6_internal_ | GAUAUUGUGACAUAUCUCUGCACUCAU |
| | loop-symmetric_ | CACACAAAAGAUGCAAAUCUCCACCCG |
| | AGAGAU-UUGGUA | GAUCCACAGGGAGGGGGCAUCUUAAU |
| | 5_1-1_mismatch_ | AUUUGGUAGAACUAAUCAUC |
| | A-C | |
| | 27_6-6_internal_ | |
| | loop-symmetric_ | |
| | UAUAGA-CCACCC | |
| 1253 | -3_6-6_internal_ | GAUAUUGUGACAUAUCUCUGCACUCAU |
| | loop-symmetric_ | CACACAAAAGAUGCAAAUCUAGCACCG |
| | AGAGAU-CCGCAA | GAUCCACAGGGAGGGGGCAUUUUAAU |
| | 27_6-6_internal_ | AUCCGCAAGAACUAAUCAUC |
| | loop-symmetric_ | |
| | UAUAGA-AGCACC | |
| 1254 | -3_6-6_internal_ | GAUAUUGUGACAUAUCUCUGCACUCAU |
| | loop-symmetric_ | CACACAAAAGAUGCAAAUCAUACCGAG |
| | AGAGAU-UCCAGG | GAUCCACAGGGAGGGGGCAUUUUAAC |
| | 0_1-1_mismatch_ | AUUCCAGGGAACUAAUCAUC |
| | A-C | |
| | 28_6-6_internal_ | |
| | loop-symmetric_ | |
| | AUAGAA-AUACCG | |
| 1255 | -3_6-6_internal_ | GAUAUUGUGACAUAUCUCUGCACUCAU |
| | loop-symmetric_ | CACACAAAAGAUGCAAAUCGAAUCAAG |
| | AGAGAU-UAGCGC | GAUCCACAGGGAGGGGGCAUUUCAAU |
| | 3_1-1_mismatch_ | AUUAGCGCGAACUAAUCAUC |
| | A-C | |
| | 28_6-6_internal_ | |
| | loop-symmetric_ | |
| | AUAGAA-GAAUCA | |
| 1256 | -3_6-6_internal_ | GAUAUUGUGACAUAUCUCUGCACUCAU |
| | loop-symmetric_ | CACACAAAAGAUGCAAAUCGGUCACAG |
| | AGAGAU-CCACCA | GAUCCACAGGGAGGGGGCAUUCUAAU |
| | 4_1-1_mismatch_ | AUCCACCAGAACUAAUCAUC |
| | A-C | |
| | 28_6-6_internal_ | |
| | loop-symmetric_ | |
| | AUAGAA-GGUCAC | |
| 1257 | -3_6-6_internal_ | GAUAUUGUGACAUAUCUCUGCACUCAU |
| | loop-symmetric_ | CACACAAAAGAUGCAAAUCCAUCAGAG |
| | AGAGAU-UUUAGG | GAUCCACAGGGAGGGGGCAUCUUAAU |
| | 5_1-1_mismatch_ | AUUUUAGGGAACUAAUCAUC |
| | A-C | |
| | 28_6-6_internal_ | |
| | loop-symmetric_ | |
| | AUAGAA-CAUCAG | |
| 1258 | -3_6-6_internal_ | GAUAUUGUGACAUAUCUCUGCACUCAU |
| | loop-symmetric_ | CACACAAAAGAUGCAAAUCCGACGGAG |
| | AGAGAU-UCAGGG | GAUCCACAGGGAGGGGGCAUUUUAAU |
| | 28_6-6_internal_ | AUUCAGGGGAACUAAUCAUC |
| | loop-symmetric_ | |
| | AUAGAA-CGACGG | |
| 1259 | -3_6-6_internal_ | GAUAUUGUGACAUAUCUCUGCACUCAU |
| | loop-symmetric_ | CACACAAAAGAUGCAAAUAUUAGCUA |
| | AGAGAU-UAACAA | GGAUCCACAGGGAGGGGGCAUUUUAA |
| | 0_1-1_mismatch_ | CAUUAACAAGAACUAAUCAUC |
| | A-C | |
| | 29_6-6_internal_ | |
| | loop-symmetric_ | |
| | UAGAAG-AUUAGC | |
| 1260 | -3_6-6_internal_ | GAUAUUGUGACAUAUCUCUGCACUCAU |
| | loop-symmetric_ | CACACAAAAGAUGCAAAUAAGAGUUA |
| | AGAGAU-CCGAAC | GGAUCCACAGGGAGGGGGCAUUUCAA |
| | 3_1-1_mismatch_ | UAUCCGAACGAACUAAUCAUC |
| | A-C | |
| | 29_6-6_internal_ | |
| | loop-symmetric_ | |
| | UAGAAG-AAGAGU | |
| 1261 | -3_6-6_internal_ | GAUAUUGUGACAUAUCUCUGCACUCAU |
| | loop-symmetric_ | CACACAAAAGAUGCAAAUGCCACCUAG |
| | AGAGAU-UAGGGA | GAUCCACAGGGAGGGGGCAUUCUAAU |
| | 4_1-1_mismatch_ | AUUAGGGAGAACUAAUCAUC |
| | A-C | |
| | 29_6-6_internal_ | |
| | loop-symmetric_ | |
| | UAGAAG-GCCACC | |
| 1262 | -3_6-6_internal_ | GAUAUUGUGACAUAUCUCUGCACUCAU |
| | loop-symmetric_ | CACACAAAAGAUGCAAAUAAUACUUA |
| | AGAGAU-UAGCGA | GGAUCCACAGGGAGGGGGCAUCUUAA |
| | 5_1-1_mismatch_ | UAUUAGCGAGAACUAAUCAUC |
| | A-C | |
| | 29_6-6_internal_ | |
| | loop-symmetric_ | |
| | UAGAAG-AAUACU | |
| 1263 | -3_6-6_internal_ | GAUAUUGUGACAUAUCUCUGCACUCAU |
| | loop-symmetric_ | CACACAAAAGAUGCAAAUGACGUUUA |
| | AGAGAU-CAAGGA | GGAUCCACAGGGAGGGGGCAUUUUAA |
| | 29_6-6_internal_ | UAUCAAGGAGAACUAAUCAUC |
| | loop-symmetric_ | |
| | UAGAAG-GACGUU | |
| 1264 | -3_6-6_internal_ | GAUAUUGUGACAUAUCUCUGCACUCAU |
| | loop-symmetric_ | CACACAAAAGAUGCAAACAUAUGAUA |
| | AGAGAU-UUAAUA | GGAUCCACAGGGAGGGGGCAUUUUAA |
| | 0_1-1_mismatch_ | CAUUUAAUAGAACUAAUCAUC |
| | A-C | |
| | 30_6-6_internal_ | |
| | loop-symmetric_ | |
| | AGAAGA-CAUAUG | |
| 1265 | -3_6-6_internal_ | GAUAUUGUGACAUAUCUCUGCACUCAU |
| | loop-symmetric_ | CACACAAAAGAUGCAAAAGUCAAAUA |
| | AGAGAU-CCUGCG | GGAUCCACAGGGAGGGGGCAUUUCAA |
| | 3_1-1_mismatch_ | UAUCCUGCGGAACUAAUCAUC |
| | A-C | |
| | 30_6-6_internal_ | |
| | loop-symmetric_ | |
| | AGAAGA-AGUCAA | |
| 1266 | -3_6-6_internal_ | GAUAUUGUGACAUAUCUCUGCACUCAU |
| | loop-symmetric_ | CACACAAAAGAUGCAAACCCCGCAUAG |
| | AGAGAU-CCGAGA | GAUCCACAGGGAGGGGGCAUUCUAAU |
| | 4_1-1_mismatch_ | AUCCGAGAGAACUAAUCAUC |
| | A-C | |
| | 30_6-6_internal_ | |
| | loop-symmetric_ | |
| | AGAAGA-CCCCGC | |
| 1267 | -3_6-6_internal_ | GAUAUUGUGACAUAUCUCUGCACUCAU |
| | loop-symmetric_ | CACACAAAAGAUGCAAACACGAAAUAG |
| | AGAGAU-CAAGUA | GAUCCACAGGGAGGGGGCAUCUUAAU |
| | 5_1-1_mismatch_ | AUCAAGUAGAACUAAUCAUC |
| | A-C | |
| | 30_6-6_internal_ | |
| | loop-symmetric_ | |
| | AGAAGA-CACGAA | |
| 1268 | -3_6-6_internal_ | GAUAUUGUGACAUAUCUCUGCACUCAU |
| | loop-symmetric_ | CACACAAAAGAUGCAAAGCCAGAAUAG |
| | AGAGAU-CGAGUG | GAUCCACAGGGAGGGGGCAUUUUAAU |
| | 30_6-6_internal_ | AUCGAGUGGAACUAAUCAUC |
| | loop-symmetric_ | |
| | AGAAGA-GCCAGA | |
| 1269 | -3_6-6_internal_ | GAUAUUGUGACAUAUCUCUGCACUCAU |
| | loop-symmetric_ | CACACAAAAGAUGCAAUGGGGAUAUA |
| | AGAGAU-UGGCGA | GGAUCCACAGGGAGGGGGCAUUUUAA |
| | 0_1-1_mismatch_ | CAUUGGCGAGAACUAAUCAUC |
| | A-C | |
| | 31_6-6_internal_ | |
| | loop-symmetric_ | |
| | GAAGAU-UGGGGA | |
| 1270 | -3_6-6_internal_ | GAUAUUGUGACAUAUCUCUGCACUCAU |
| | loop-symmetric_ | CACACAAAAGAUGCAACUGGUAUAUA |
| | AGAGAU-UAACGC | GGAUCCACAGGGAGGGGGCAUUUCAA |
| | 3_1-1_mismatch_ | UAUUAACGCGAACUAAUCAUC |
| | A-C | |
| | 31_6-6_internal_ | |
| | loop-symmetric_ | |
| | GAAGAU-CUGGUA | |
| 1271 | -3_6-6_internal_ | GAUAUUGUGACAUAUCUCUGCACUCAU |
| | loop-symmetric_ | CACACAAAAGAUGCAACGGGAAUAUA |
| | AGAGAU-CCACCA | GGAUCCACAGGGAGGGGGCAUUCUAA |
| | 4_1-1_mismatch_ | UAUCCACCAGAACUAAUCAUC |
| | A-C | |
| | 31_6-6_internal_ | |
| | loop-symmetric_ | |
| | GAAGAU-CGGGAA | |
| 1272 | -3_6-6_internal_ | GAUAUUGUGACAUAUCUCUGCACUCAU |
| | loop-symmetric_ | CACACAAAAGAUGCAAUCAACGUAUAG |
| | AGAGAU-UUGAGA | GAUCCACAGGGAGGGGGCAUCUUAAU |
| | 5_1-1_mismatch_ | AUUUGAGAGAACUAAUCAUC |
| | A-C | |
| | 31_6-6_internal_ | |
| | loop-symmetric_ | |
| | GAAGAU-UCAACG | |
| 1273 | -3_6-6_internal_ | GAUAUUGUGACAUAUCUCUGCACUCAU |
| | loop-symmetric_ | CACACAAAAGAUGCAUUGACCCUAUAG |
| | AGAGAU-UAGCCG | GAUCCACAGGGAGGGGGCAUUUUAAC |
| | 0_1-1_mismatch_ | AUUAGCCGGAACUAAUCAUC |
| | A-C | |
| | 32_6-6_internal_ | |
| | loop-symmetric_ | |
| | AAGAUU-UUGACC | |
| 1274 | -3_6-6_internal_ | GAUAUUGUGACAUAUCUCUGCACUCAU |
| | loop-symmetric_ | CACACAAAAGAUGCAUAAAACCUAUAG |
| | AGAGAU-CGUAUG | GAUCCACAGGGAGGGGGCAUUUCAAU |
| | 3_1-1_mismatch_ | AUCGUAUGGAACUAAUCAUC |
| | A-C | |
| | 32_6-6_internal_ | |
| | loop-symmetric_ | |
| | AAGAUU-UAAAAC | |
| 1275 | -3_6-6_internal_ | GAUAUUGUGACAUAUCUCUGCACUCAU |
| | loop-symmetric_ | CACACAAAAGAUGCAUUCAUGCUAUAG |
| | AGAGAU-UAAACA | GAUCCACAGGGAGGGGGCAUUCUAAU |
| | 4_1-1_mismatch_ | AUUAAACAGAACUAAUCAUC |
| | A-C | |
| | 32_6-6_internal_ | |
| | loop-symmetric_ | |
| | AAGAUU-UUCAUG | |
| 1276 | -3_6-6_internal_ | GAUAUUGUGACAUAUCUCUGCACUCAU |
| | loop-symmetric_ | CACACAAAAGAUGCAUGCAGCCUAUAG |
| | AGAGAU-UGUAUG | GAUCCACAGGGAGGGGGCAUCUUAAU |
| | 5_1-1_mismatch_ | AUUGUAUGGAACUAAUCAUC |
| | A-C | |
| | 32_6-6_internal_ | |
| | loop-symmetric_ | |
| | AAGAUU-UGCAGC | |
| 1277 | -3_6-6_internal_ | GAUAUUGUGACAUAUCUCUGCACUCAU |
| | loop-symmetric_ | CACACAAAAGAUGCAUUAGGGCUAUA |
| | AGAGAU-UCAGGA | GGAUCCACAGGGAGGGGGCAUUUUAA |
| | 32_6-6_internal_ | UAUUCAGGAGAACUAAUCAUC |
| | loop-symmetric_ | |
| | AAGAUU-UUAGGG | |
| 1278 | -3_6-6_internal_ | GAUAUUGUGACAUAUCUCUGCACUCAU |
| | loop-symmetric_ | CACACAAAAGAUGCCCCUACUCUAUAG |
| | AGAGAU-UGUAGA | GAUCCACAGGGAGGGGGCAUUUUAAC |
| | 0_1-1_mismatch_ | AUUGUAGAGAACUAAUCAUC |
| | A-C | |
| | 33_6-6_internal_ | |
| | loop-symmetric_ | |
| | AGAUUU-CCCUAC | |
| 1279 | -3_6-6_internal_ | GAUAUUGUGACAUAUCUCUGCACUCAU |
| | loop-symmetric_ | CACACAAAAGAUGCUUACAAUCUAUAG |
| | AGAGAU-CCUGCG | GAUCCACAGGGAGGGGGCAUUUCAAU |
| | 3_1-1_mismatch_ | AUCCUGCGGAACUAAUCAUC |
| | A-C | |
| | 33_6-6_internal_ | |
| | loop-symmetric_ | |
| | AGAUUU-UUACAA | |
| 1280 | -3_6-6_internal_ | GAUAUUGUGACAUAUCUCUGCACUCAU |
| | loop-symmetric_ | CACACAAAAGAUGCUCGGGCUCUAUAG |
| | AGAGAU-UGACCG | GAUCCACAGGGAGGGGGCAUCUUAAU |
| | 5_1-1_mismatch_ | AUUGACCGGAACUAAUCAUC |
| | A-C | |
| | 33_6-6_internal_ | |
| | loop-symmetric_ | |
| | AGAUUU-UCGGGC | |
| 1281 | -3_6-6_internal_ | GAUAUUGUGACAUAUCUCUGCACUCAU |
| | loop-symmetric_ | CACACAAAAGAUGCCCAACCUCUAUAG |
| | AGAGAU-CAAUGG | GAUCCACAGGGAGGGGGCAUUUUAAU |
| | 33_6-6_internal_ | AUCAAUGGGAACUAAUCAUC |
| | loop-symmetric_ | |
| | AGAUUU-CCAACC | |
| 1282 | -3_6-6_internal_ | GAUAUUGUGACAUAUCUCUGCACUCAU |
| | loop-symmetric_ | CACACAAAAGAUGGUUUAGUUCUAUA |
| | AGAGAU-UGGCGG | GGAUCCACAGGGAGGGGGCAUUUUAA |
| | 0_1-1_mismatch_ | CAUUGGCGGGAACUAAUCAUC |
| | A-C | |
| | 34_6-6_internal_ | |
| | loop-symmetric_ | |
| | GAUUUG-GUUUAG | |
| 1283 | -3_6-6_internal_ | GAUAUUGUGACAUAUCUCUGCACUCAU |
| | loop-symmetric_ | CACACAAAAGAUGACUGCGUUCUAUAG |
| | AGAGAU-UCAGAA | GAUCCACAGGGAGGGGGCAUUUCAAU |
| | 3_1-1_mismatch_ | AUUCAGAAGAACUAAUCAUC |
| | A-C | |
| | 34_6-6_internal_ | |
| | loop-symmetric_ | |
| | GAUUUG-ACUGCG | |
| 1284 | -3_6-6_internal_ | GAUAUUGUGACAUAUCUCUGCACUCAU |
| | loop-symmetric_ | CACACAAAAGAUGAUACGGUUCUAUA |
| | AGAGAU-UUGAAA | GGAUCCACAGGGAGGGGGCAUUCUAA |
| | 4_1-1_mismatch_ | UAUUUGAAAGAACUAAUCAUC |
| | A-C | |
| | 34_6-6_internal_ | |
| | loop-symmetric_ | |
| | GAUUUG-AUACGG | |
| 1285 | -3_6-6_internal_ | GAUAUUGUGACAUAUCUCUGCACUCAU |
| | loop-symmetric_ | CACACAAAAGAUGGCGUGAUUCUAUA |
| | AGAGAU-CCAAUG | GGAUCCACAGGGAGGGGGCAUCUUAA |
| | 5_1-1_mismatch_ | UAUCCAAUGGAACUAAUCAUC |
| | A-C | |
| | 34_6-6_internal_ | |
| | loop-symmetric_ | |
| | GAUUUG-GCGUGA | |
| 1286 | -3_6-6_internal_ | GAUAUUGUGACAUAUCUCUGCACUCAU |
| | loop-symmetric_ | CACACAAAAGAUGAGUAAAUUCUAUA |
| | AGAGAU-CGAACG | GGAUCCACAGGGAGGGGGCAUUUUAA |
| | 34_6-6_internal_ | UAUCGAACGGAACUAAUCAUC |
| | loop-symmetric_ | |
| | GAUUUG-AGUAAA | |
| 1287 | -3_6-6_internal_ | GAUAUUGUGACAUAUCUCUGCACUCAU |
| | loop-symmetric_ | CACACAAAAGAUAAUAUACUUCUAUA |
| | AGAGAU-UAGGAC | GGAUCCACAGGGAGGGGGCAUUUUAA |
| | 0_1-1_mismatch_ | CAUUAGGACGAACUAAUCAUC |
| | A-C | |
| | 35_6-6_internal_ | |
| | loop-symmetric_ | |
| | AUUUGC-AAUAUA | |
| 1288 | -3_6-6_internal_ | GAUAUUGUGACAUAUCUCUGCACUCAU |
| | loop-symmetric_ | CACACAAAAGAUCUUCGCCUUCUAUAG |
| | AGAGAU-CAUAGG | GAUCCACAGGGAGGGGGCAUUUCAAU |
| | 3_1-1_mismatch_ | AUCAUAGGGAACUAAUCAUC |
| | A-C | |
| | 35_6-6_internal_ | |
| | loop-symmetric_ | |
| | AUUUGC-CUUCGC | |
| 1289 | -3_6-6_internal_ | GAUAUUGUGACAUAUCUCUGCACUCAU |
| | loop-symmetric_ | CACACAAAAGAUCUCCGCCUUCUAUAG |
| | AGAGAU-CAGCGA | GAUCCACAGGGAGGGGGCAUUCUAAU |
| | 4_1-1_mismatch_ | AUCAGCGAGAACUAAUCAUC |
| | A-C | |
| | 35_6-6_internal_ | |
| | loop-symmetric_ | |
| | AUUUGC-CUCCGC | |
| 1290 | -3_6-6_internal_ | GAUAUUGUGACAUAUCUCUGCACUCAU |
| | loop-symmetric_ | CACACAAAAGAUAUUGUGCUUCUAUA |
| | AGAGAU-UCACAG | GGAUCCACAGGGAGGGGGCAUCUUAA |
| | 5_1-1_mismatch_ | UAUUCACAGGAACUAAUCAUC |
| | A-C | |
| | 35_6-6_internal_ | |
| | loop-symmetric_ | |
| | AUUUGC-AUUGUG | |
| 1291 | -3_6-6_internal_ | GAUAUUGUGACAUAUCUCUGCACUCAU |
| | loop-symmetric_ | CACACAAAAGAUCUACACCUUCUAUAG |
| | AGAGAU-CAGGAG | GAUCCACAGGGAGGGGGCAUUUUAAU |
| | 35_6-6_internal_ | AUCAGGAGGAACUAAUCAUC |
| | loop-symmetric_ | |
| | AUUUGC-CUACAC | |
| 1292 | -3_6-6_internal_ | GAUAUUGUGACAUAUCUCUGCACUCAU |
| | loop-symmetric_ | CACACAAAAGAGUCUACUCUUCUAUAG |
| | AGAGAU-UAAGUA | GAUCCACAGGGAGGGGGCAUUUUAAC |
| | 0_1-1_mismatch_ | AUUAAGUAGAACUAAUCAUC |
| | A-C | |
| | 36_6-6_internal_ | |
| | loop-symmetric_ | |
| | UUUGCA-GUCUAC | |
| 1293 | -3_6-6_internal_ | GAUAUUGUGACAUAUCUCUGCACUCAU |
| | loop-symmetric_ | CACACAAAAGAGGGUCCUCUUCUAUAG |
| | AGAGAU-CCAGGG | GAUCCACAGGGAGGGGGCAUUUCAAU |
| | 3_1-1_mismatch_ | AUCCAGGGGAACUAAUCAUC |
| | A-C | |
| | 36_6-6_internal_ | |
| | loop-symmetric_ | |
| | UUUGCA-GGGUCC | |
| 1294 | -3_6-6_internal_ | GAUAUUGUGACAUAUCUCUGCACUCAU |
| | loop-symmetric_ | CACACAAAAGACCCCUCUCUUCUAUAG |
| | AGAGAU-UCAAAC | GAUCCACAGGGAGGGGGCAUUCUAAU |
| | 4_1-1_mismatch_ | AUUCAAACGAACUAAUCAUC |
| | A-C | |
| | 36_6-6_internal_ | |
| | loop-symmetric_ | |
| | UUUGCA-CCCCUC | |
| 1295 | -3_6-6_internal_ | GAUAUUGUGACAUAUCUCUGCACUCAU |
| | loop-symmetric_ | CACACAAAAGAACAUUCUCUUCUAUAG |
| | AGAGAU-CACAGC | GAUCCACAGGGAGGGGGCAUCUUAAU |
| | 5_1-1_mismatch_ | AUCACAGCGAACUAAUCAUC |
| | A-C | |
| | 36_6-6_internal_ | |
| | loop-symmetric_ | |
| | UUUGCA-ACAUUC | |
| 1296 | -3_6-6_internal_ | GAUAUUGUGACAUAUCUCUGCACUCAU |
| | loop-symmetric_ | CACACAAAAGAAAAACCUCUUCUAUAG |
| | AGAGAU-UGGAUG | GAUCCACAGGGAGGGGGCAUUUUAAU |
| | 36_6-6_internal_ | AUUGGAUGGAACUAAUCAUC |
| | loop-symmetric_ | |
| | UUUGCA-AAAACC | |
| 1297 | -3_6-6_internal_ | GAUAUUGUGACAUAUCUCUGCACUCAU |
| | loop-symmetric_ | CACACAAAAGUCCGUUAUCUUCUAUAG |
| | AGAGAU-UACAAC | GAUCCACAGGGAGGGGGCAUUUUAAC |
| | 0_1-1_mismatch_ | AUUACAACGAACUAAUCAUC |
| | A-C | |
| | 37_6-6_internal_ | |
| | loop-symmetric_ | |
| | UUGCAU-UCCGUU | |
| 1298 | -3_6-6_internal_ | GAUAUUGUGACAUAUCUCUGCACUCAU |
| | loop-symmetric_ | CACACAAAAGUACUUCAUCUUCUAUAG |
| | AGAGAU-UGGCAC | GAUCCACAGGGAGGGGGCAUUUCAAU |
| | 3_1-1_mismatch_ | AUUGGCACGAACUAAUCAUC |
| | A-C | |
| | 37_6-6_internal_ | |
| | loop-symmetric_ | |
| | UUGCAU-UACUUC | |
| 1299 | -3_6-6_internal_ | GAUAUUGUGACAUAUCUCUGCACUCAU |
| | loop-symmetric_ | CACACAAAAGCAAAUUAUCUUCUAUAG |
| | AGAGAU-UGGGGA | GAUCCACAGGGAGGGGGCAUUCUAAU |
| | 4_1-1_mismatch_ | AUUGGGGAGAACUAAUCAUC |
| | A-C | |
| | 37_6-6_internal_ | |
| | loop-symmetric_ | |
| | UUGCAU-CAAAUU | |
| 1300 | -3_6-6_internal_ | GAUAUUGUGACAUAUCUCUGCACUCAU |
| | loop-symmetric_ | CACACAAAAGUAAGAUAUCUUCUAUA |
| | AGAGAU-CCAGUG | GGAUCCACAGGGAGGGGGCAUCUUAA |
| | 5_1-1_mismatch_ | UAUCCAGUGGAACUAAUCAUC |
| | A-C | |
| | 37_6-6_internal_ | |
| | loop-symmetric_ | |
| | UUGCAU-UAAGAU | |
| 1301 | -3_6-6_internal_ | GAUAUUGUGACAUAUCUCUGCACUCAU |
| | loop-symmetric_ | CACACAAAAGCGUUUUAUCUUCUAUAG |
| | AGAGAU-UAGUAC | GAUCCACAGGGAGGGGGCAUUUUAAU |
| | 37_6-6_internal_ | AUUAGUACGAACUAAUCAUC |
| | loop-symmetric_ | |
| | UUGCAU-CGUUUU | |
| 1302 | -3_6-6_internal_ | GAUAUUGUGACAUAUCUCUGCACUCAU |
| | loop-symmetric_ | CACACAAAAUUAUUCAAUCUUCUAUAG |
| | AGAGAU-UAACGA | GAUCCACAGGGAGGGGGCAUUUUAAC |
| | 0_1-1_mismatch_ | AUUAACGAGAACUAAUCAUC |
| | A-C | |
| | 38_6-6_internal_ | |
| | loop-symmetric_ | |
| | UGCAUC-UUAUUC | |
| 1303 | -3_6-6_internal_ | GAUAUUGUGACAUAUCUCUGCACUCAU |
| | loop-symmetric_ | CACACAAAAAUCUCCAAUCUUCUAUAG |
| | AGAGAU-CAAGGG | GAUCCACAGGGAGGGGGCAUUUCAAU |
| | 3_1-1_mismatch_ | AUCAAGGGGAACUAAUCAUC |
| | A-C | |
| | 38_6-6_internal_ | |
| | loop-symmetric_ | |
| | UGCAUC-AUCUCC | |
| 1304 | -3_6-6_internal_ | GAUAUUGUGACAUAUCUCUGCACUCAU |
| | loop-symmetric_ | CACACAAAACUAUCUAAUCUUCUAUAG |
| | AGAGAU-UUGGAC | GAUCCACAGGGAGGGGGCAUUCUAAU |
| | 4_1-1_mismatch_ | AUUUGGACGAACUAAUCAUC |
| | A-C | |
| | 38_6-6_internal_ | |
| | loop-symmetric_ | |
| | UGCAUC-CUAUCU | |
| 1305 | -3_6-6_internal_ | GAUAUUGUGACAUAUCUCUGCACUCAU |
| | loop-symmetric_ | CACACAAAACCUAGUAAUCUUCUAUAG |
| | AGAGAU-UUGAGA | GAUCCACAGGGAGGGGGCAUCUUAAU |
| | 5_1-1_mismatch_ | AUUUGAGAGAACUAAUCAUC |
| | A-C | |
| | 38_6-6_internal_ | |
| | loop-symmetric_ | |
| | UGCAUC-CCUAGU | |
| 1306 | -3_6-6_internal_ | GAUAUUGUGACAUAUCUCUGCACUCAU |
| | loop-symmetric_ | CACACAAAACAACGUAAUCUUCUAUAG |
| | AGAGAU-CGAGAA | GAUCCACAGGGAGGGGGCAUUUUAAU |
| | 38_6-6_internal_ | AUCGAGAAGAACUAAUCAUC |
| | loop-symmetric_ | |
| | UGCAUC-CAACGU | |
| 1307 | -3_6-6_internal_ | GAUAUUGUGACAUAUCUCUGCACUCAU |
| | loop-symmetric_ | CACACAAACGCAAAAAAUCUUCUAUAG |
| | AGAGAU-UGUAAC | GAUCCACAGGGAGGGGGCAUUUUAAC |
| | 0_1-1_mismatch_ | AUUGUAACGAACUAAUCAUC |
| | A-C | |
| | 39_6-6_internal_ | |
| | loop-symmetric_ | |
| | GCAUCU-CGCAAA | |
| 1308 | -3_6-6_internal_ | GAUAUUGUGACAUAUCUCUGCACUCAU |
| | loop-symmetric_ | CACACAAAUAUCAGAAAUCUUCUAUAG |
| | AGAGAU-CGAGAC | GAUCCACAGGGAGGGGGCAUUUCAAU |
| | 3_1-1_mismatch_ | AUCGAGACGAACUAAUCAUC |
| | A-C | |
| | 39_6-6_internal_ | |
| | loop-symmetric_ | |
| | GCAUCU-UAUCAG | |
| 1309 | -3_6-6_internal_ | GAUAUUGUGACAUAUCUCUGCACUCAU |
| | loop-symmetric_ | CACACAAACACUAGAAAUCUUCUAUAG |
| | AGAGAU-CCGAGC | GAUCCACAGGGAGGGGGCAUUCUAAU |
| | 4_1-1_mismatch_ | AUCCGAGCGAACUAAUCAUC |
| | A-C | |
| | 39_6-6_internal_ | |
| | loop-symmetric_ | |
| | GCAUCU-CACUAG | |
| 1310 | -3_6-6_internal_ | GAUAUUGUGACAUAUCUCUGCACUCAU |
| | loop-symmetric_ | CACACAAAUUAUUAAAAUCUUCUAUA |
| | AGAGAU-UGGGUG | GGAUCCACAGGGAGGGGGCAUCUUAA |
| | 5_1-1_mismatch_ | UAUUGGGUGGAACUAAUCAUC |
| | A-C | |
| | 39_6-6_internal_ | |
| | loop-symmetric_ | |
| | GCAUCU-UUAUUA | |
| 1311 | -3_6-6_internal_ | GAUAUUGUGACAUAUCUCUGCACUCAU |
| | loop-symmetric_ | CACACAAAUCAACGAAAUCUUCUAUAG |
| | AGAGAU-CCAAGC | GAUCCACAGGGAGGGGGCAUUUUAAU |
| | 39_6-6_internal_ | AUCCAAGCGAACUAAUCAUC |
| | loop-symmetric_ | |
| | GCAUCU-UCAACG | |
| 1312 | -3_6-6_internal_ | GAUAUUGUGACAUAUCUCUGCACUCAU |
| | loop-symmetric_ | CACACAAUCACCCCAAAUCUUCUAUAG |
| | AGAGAU-UAGACG | GAUCCACAGGGAGGGGGCAUUUUAAC |
| | 0_1-1_mismatch_ | AUUAGACGGAACUAAUCAUC |
| | A-C | |
| | 40_6-6_internal_ | |
| | loop-symmetric_ | |
| | CAUCUU-UCACCC | |
| 1313 | -3_6-6_internal_ | GAUAUUGUGACAUAUCUCUGCACUCAU |
| | loop-symmetric_ | CACACAACCUAAUCAAAUCUUCUAUAG |
| | AGAGAU-CGACGC | GAUCCACAGGGAGGGGGCAUUUCAAU |
| | 3_1-1_mismatch_ | AUCGACGCGAACUAAUCAUC |
| | A-C | |
| | 40_6-6_internal_ | |
| | loop-symmetric_ | |
| | CAUCUU-CCUAAU | |
| 1314 | -3_6-6_internal_ | GAUAUUGUGACAUAUCUCUGCACUCAU |
| | loop-symmetric_ | CACACAACUCCAUCAAAUCUUCUAUAG |
| | AGAGAU-CGACCA | GAUCCACAGGGAGGGGGCAUUCUAAU |
| | 4_1-1_mismatch_ | AUCGACCAGAACUAAUCAUC |
| | A-C | |
| | 40_6-6_internal_ | |
| | loop-symmetric_ | |
| | CAUCUU-CUCCAU | |
| 1315 | -3_6-6_internal_ | GAUAUUGUGACAUAUCUCUGCACUCAU |
| | loop-symmetric_ | CACACAACAAUUUCAAAUCUUCUAUAG |
| | AGAGAU-CAGCAA | GAUCCACAGGGAGGGGGCAUCUUAAU |
| | 5_1-1_mismatch_ | AUCAGCAAGAACUAAUCAUC |
| | A-C | |
| | 40_6-6_internal_ | |
| | loop-symmetric_ | |
| | CAUCUU-CAAUUU | |
| 1316 | -3_6-6_internal_ | GAUAUUGUGACAUAUCUCUGCACUCAU |
| | loop-symmetric_ | CACACAACCUGCCCAAAUCUUCUAUAG |
| | AGAGAU-UCCAGA | GAUCCACAGGGAGGGGGCAUUUUAAU |
| | 40_6-6_internal_ | AUUCCAGAGAACUAAUCAUC |
| | loop-symmetric_ | |
| | CAUCUU-CCUGCC | |
| 1317 | -3_6-6_internal_ | GAUAUUGUGACAUAUCUCUGCACUCAU |
| | loop-symmetric_ | CACACACCGUGCGCAAAUCUUCUAUAG |
| | AGAGAU-UACAAC | GAUCCACAGGGAGGGGGCAUUUUAAC |
| | 0_1-1_mismatch_ | AUUACAACGAACUAAUCAUC |
| | A-C | |
| | 41_6-6_internal_ | |
| | loop-symmetric_ | |
| | AUCUUU-CCGUGC | |
| 1318 | -3_6-6_internal_ | GAUAUUGUGACAUAUCUCUGCACUCAU |
| | loop-symmetric_ | CACACAUUAUACGCAAAUCUUCUAUAG |
| | AGAGAU-UUACGG | GAUCCACAGGGAGGGGGCAUUUCAAU |
| | 3_1-1_mismatch_ | AUUUACGGGAACUAAUCAUC |
| | A-C | |
| | 41_6-6_internal_ | |
| | loop-symmetric_ | |
| | AUCUUU-UUAUAC | |
| 1319 | -3_6-6_internal_ | GAUAUUGUGACAUAUCUCUGCACUCAU |
| | loop-symmetric_ | CACACACUAACCGCAAAUCUUCUAUAG |
| | AGAGAU-UAGCAC | GAUCCACAGGGAGGGGGCAUUCUAAU |
| | 4_1-1_mismatch_ | AUUAGCACGAACUAAUCAUC |
| | A-C | |
| | 41_6-6_internal_ | |
| | loop-symmetric_ | |
| | AUCUUU-CUAACC | |
| 1320 | -3_6-6_internal_ | GAUAUUGUGACAUAUCUCUGCACUCAU |
| | loop-symmetric_ | CACACAUUUGUAGCAAAUCUUCUAUAG |
| | AGAGAU-CAAGGC | GAUCCACAGGGAGGGGGCAUCUUAAU |
| | 5_1-1_mismatch_ | AUCAAGGCGAACUAAUCAUC |
| | A-C | |
| | 41_6-6_internal_ | |
| | loop-symmetric_ | |
| | AUCUUU-UUUGUA | |
| 1321 | -3_6-6_internal_ | GAUAUUGUGACAUAUCUCUGCACUCAU |
| | loop-symmetric_ | CACACAUGCUCAGCAAAUCUUCUAUAG |
| | AGAGAU-CAGUGG | GAUCCACAGGGAGGGGGCAUUUUAAU |
| | 41_6-6_internal_ | AUCAGUGGGAACUAAUCAUC |
| | loop-symmetric_ | |
| | AUCUUU-UGCUCA | |
| 1322 | -3_6-6_internal_ | GAUAUUGUGACAUAUCUCUGCACUCAU |
| | loop-symmetric_ | CACACUCCUAUUGCAAAUCUUCUAUAG |
| | AGAGAU-UUAGUG | GAUCCACAGGGAGGGGGCAUUUUAAC |
| | 0_1-1_mismatch_ | AUUUAGUGGAACUAAUCAUC |
| | A-C | |
| | 42_6-6_internal_ | |
| | loop-symmetric_ | |
| | UCUUUU-UCCUAU | |
| 1323 | -3_6-6_internal_ | GAUAUUGUGACAUAUCUCUGCACUCAU |
| | loop-symmetric_ | CACACUGUACCUGCAAAUCUUCUAUAG |
| | AGAGAU-CAGCAG | GAUCCACAGGGAGGGGGCAUUUCAAU |
| | 3_1-1_mismatch_ | AUCAGCAGGAACUAAUCAUC |
| | A-C | |
| | 42_6-6_internal_ | |
| | loop-symmetric_ | |
| | UCUUUU-UGUACC | |
| 1324 | -3_6-6_internal_ | GAUAUUGUGACAUAUCUCUGCACUCAU |
| | loop-symmetric_ | CACACUCCCUCUGCAAAUCUUCUAUAG |
| | AGAGAU-UCCAAA | GAUCCACAGGGAGGGGGCAUUCUAAU |
| | 4_1-1_mismatch_ | AUUCCAAAGAACUAAUCAUC |
| | A-C | |
| | 42_6-6_internal_ | |
| | loop-symmetric_ | |
| | UCUUUU-UCCCUC | |
| 1325 | -3_6-6_internal_ | GAUAUUGUGACAUAUCUCUGCACUCAU |
| | loop-symmetric_ | CACACUUCCGUUGCAAAUCUUCUAUAG |
| | AGAGAU-CCGCAA | GAUCCACAGGGAGGGGGCAUCUUAAU |
| | 5_1-1_mismatch_ | AUCCGCAAGAACUAAUCAUC |
| | A-C | |
| | 42_6-6_internal_ | |
| | loop-symmetric_ | |
| | UCUUUU-UUCCGU | |
| 1326 | -3_6-6_internal_ | GAUAUUGUGACAUAUCUCUGCACUCAU |
| | loop-symmetric_ | CACACCGGCCUUGCAAAUCUUCUAUAG |
| | AGAGAU-CAAGGA | GAUCCACAGGGAGGGGGCAUUUUAAU |
| | 42_6-6_internal_ | AUCAAGGAGAACUAAUCAUC |
| | loop-symmetric_ | |
| | UCUUUU-CGGCCU | |
| 1327 | -3_6-6_internal_ | GAUAUUGUGACAUAUCUCUGCACUCAU |
| | loop-symmetric_ | CACAAUGGCAAUGCAAAUCUUCUAUAG |
| | AGAGAU-UAACAA | GAUCCACAGGGAGGGGGCAUUUUAAC |
| | 0_1-1_mismatch_ | AUUAACAAGAACUAAUCAUC |
| | A-C | |
| | 43_6-6_internal_ | |
| | loop-symmetric_ | |
| | CUUUUG-AUGGCA | |
| 1328 | -3_6-6_internal_ | GAUAUUGUGACAUAUCUCUGCACUCAU |
| | loop-symmetric_ | CACAAAUCAAAUGCAAAUCUUCUAUAG |
| | AGAGAU-UUGGAC | GAUCCACAGGGAGGGGGCAUUUCAAU |
| | 3_1-1_mismatch_ | AUUUGGACGAACUAAUCAUC |
| | A-C | |
| | 43_6-6_internal_ | |
| | loop-symmetric_ | |
| | CUUUUG-AAUCAA | |
| 1329 | -3_6-6_internal_ | GAUAUUGUGACAUAUCUCUGCACUCAU |
| | loop-symmetric_ | CACAGCCAUAAUGCAAAUCUUCUAUAG |
| | AGAGAU-UACCGC | GAUCCACAGGGAGGGGGCAUUCUAAU |
| | 4_1-1_mismatch_ | AUUACCGCGAACUAAUCAUC |
| | A-C | |
| | 43_6-6_internal_ | |
| | loop-symmetric_ | |
| | CUUUUG-GCCAUA | |
| 1330 | -3_6-6_internal_ | GAUAUUGUGACAUAUCUCUGCACUCAU |
| | loop-symmetric_ | CACAGUCAAAAUGCAAAUCUUCUAUAG |
| | AGAGAU-CGAGGA | GAUCCACAGGGAGGGGGCAUCUUAAU |
| | 5_1-1_mismatch_ | AUCGAGGAGAACUAAUCAUC |
| | A-C | |
| | 43_6-6_internal_ | |
| | loop-symmetric_ | |
| | CUUUUG-GUCAAA | |
| 1331 | -3_6-6_internal_ | GAUAUUGUGACAUAUCUCUGCACUCAU |
| | loop-symmetric_ | CACAAUCCUCAUGCAAAUCUUCUAUAG |
| | AGAGAU-CAUGAG | GAUCCACAGGGAGGGGGCAUUUUAAU |
| | 43_6-6_internal_ | AUCAUGAGGAACUAAUCAUC |
| | loop-symmetric_ | |
| | CUUUUG-AUCCUC | |
| 1332 | -3_6-6_internal_ | GAUAUUGUGACAUAUCUCUGCACUCAU |
| | loop-symmetric_ | CACUAUCAUGAUGCAAAUCUUCUAUAG |
| | AGAGAU-CCGGGA | GAUCCACAGGGAGGGGGCAUUUCAAU |
| | 3_1-1_mismatch_ | AUCCGGGAGAACUAAUCAUC |
| | A-C | |
| | 44_6-6_internal_ | |
| | loop-symmetric_ | |
| | UUUUGU-UAUCAU | |
| 1333 | -3_6-6_internal_ | GAUAUUGUGACAUAUCUCUGCACUCAU |
| | loop-symmetric_ | CACUACCCUGAUGCAAAUCUUCUAUAG |
| | AGAGAU-CACCAG | GAUCCACAGGGAGGGGGCAUUCUAAU |
| | 4_1-1_mismatch_ | AUCACCAGGAACUAAUCAUC |
| | A-C | |
| | 44_6-6_internal_ | |
| | loop-symmetric_ | |
| | UUUUGU-UACCCU | |
| 1334 | -3_6-6_internal_ | GAUAUUGUGACAUAUCUCUGCACUCAU |
| | loop-symmetric_ | CACCUGUCUGAUGCAAAUCUUCUAUAG |
| | AGAGAU-CGUAUA | GAUCCACAGGGAGGGGGCAUCUUAAU |
| | 5_1-1_mismatch_ | AUCGUAUAGAACUAAUCAUC |
| | A-C | |
| | 44_6-6_internal_ | |
| | loop-symmetric_ | |
| | UUUUGU-CUGUCU | |
| 1335 | -3_6-6_internal_ | GAUAUUGUGACAUAUCUCUGCACUCAU |
| | loop-symmetric_ | CACCUGGUUGAUGCAAAUCUUCUAUAG |
| | AGAGAU-CACACG | GAUCCACAGGGAGGGGGCAUUUUAAU |
| | 44_6-6_internal_ | AUCACACGGAACUAAUCAUC |
| | loop-symmetric_ | |
| | UUUUGU-CUGGUU | |
| 1336 | -2_6-6_internal_ | GAUAUUGUGACAUAUCUCUGCACUCAU |
| | loop-symmetric_ | CACACAAAAGAUGCAAAUCUUCUAUAG |
| | GAGAUA-AUGACG | GAUCCACAGGGAGGGGGCAUUUCAAU |
| | 3_1-1_mismatch_ | AAUGACGUGAACUAAUCAUC |
| | A-C | |
| 1337 | -2_6-6_internal_ | GAUAUUGUGACAUAUCUCUGCACUCAU |
| | loop-symmetric_ | CACACAAAAGAUGCAAAUCUUCUAUAG |
| | GAGAUA-AUGAGG | GAUCCACAGGGAGGGGGCAUUCUAAU |
| | 4_1-1_mismatch_ | AAUGAGGUGAACUAAUCAUC |
| | A-C | |
| 1338 | -2_6-6_internal_ | GAUAUUGUGACAUAUCUCUGCACUCAU |
| | loop-symmetric_ | CACACAAAAGAUGCAAAUCUUCUAUAG |
| | GAGAUA-AUGAAA | GAUCCACAGGGAGGGGGCAUCUUAAU |
| | 5_1-1_mismatch_ | AAUGAAAUGAACUAAUCAUC |
| | A-C | |
| 1339 | -2_6-6_internal_ | GAUAUUGUGACAUAUCUCUGCACUCAU |
| | loop-symmetric_ | CACACAAAAGAUGCAAAUCUUCUAUAG |
| | GAGAUA-AACGCA | GAUCCACAGGGAGGGGGCAUUUUAAU |
| | | AAACGCAUGAACUAAUCAUC |
| 1340 | -2_6-6_internal_ | GAUAUUGUGACAUAUCUCUGCACUCAU |
| | loop-symmetric_ | CACACAAAAGAUGCAAAUCUUCUCCUA |
| | GAGAUA-GCCACG | CCUCCACAGGGAGGGGGCAUUUCAAUA |
| | 3_1-1_mismatch_ | GCCACGUGAACUAAUCAUC |
| | A-C | |
| | 24_6-6_internal_ | |
| | loop-symmetric_ | |
| | UCCUAU-CCUACC | |
| 1341 | -2_6-6_internal_ | GAUAUUGUGACAUAUCUCUGCACUCAU |
| | loop-symmetric_ | CACACAAAAGAUGCAAAUCUUCUCCCC |
| | GAGAUA-ACCACA | ACUCCACAGGGAGGGGGCAUUCUAAUA |
| | 4_1-1_mismatch_ | ACCACAUGAACUAAUCAUC |
| | A-C | |
| | 24_6-6_internal_ | |
| | loop-symmetric_ | |
| | UCCUAU-CCCCAC | |
| 1342 | -2_6-6_internal_ | GAUAUUGUGACAUAUCUCUGCACUCAU |
| | loop-symmetric_ | CACACAAAAGAUGCAAAUCUUCUUCCA |
| | GAGAUA-ACCAGG | UUUCCACAGGGAGGGGGCAUCUUAAU |
| | 5_1-1_mismatch_ | AACCAGGUGAACUAAUCAUC |
| | A-C | |
| | 24_6-6_internal_ | |
| | loop-symmetric_ | |
| | UCCUAU-UCCAUU | |
| 1343 | -2_6-6_internal_ | GAUAUUGUGACAUAUCUCUGCACUCAU |
| | loop-symmetric_ | CACACAAAAGAUGCAAAUCUUCUCGCU |
| | GAGAUA-CUGGAA | UCUCCACAGGGAGGGGGCAUUUUAAU |
| | 24_6-6_internal_ | ACUGGAAUGAACUAAUCAUC |
| | loop-symmetric_ | |
| | UCCUAU-CGCUUC | |
| 1344 | -2_6-6_internal_ | GAUAUUGUGACAUAUCUCUGCACUCAU |
| | loop-symmetric_ | CACACAAAAGAUGCAAAUCUUCGAUCC |
| | GAGAUA-AACAAG | UAUCCACAGGGAGGGGGCAUUUCAAU |
| | 3_1-1_mismatch_ | AAACAAGUGAACUAAUCAUC |
| | A-C | |
| | 25_6-6_internal_ | |
| | loop-symmetric_ | |
| | CCUAUA-GAUCCU | |
| 1345 | -2_6-6_internal_ | GAUAUUGUGACAUAUCUCUGCACUCAU |
| | loop-symmetric_ | CACACAAAAGAUGCAAAUCUUCGCAGU |
| | GAGAUA-CCAGCA | CAUCCACAGGGAGGGGGCAUUCUAAUA |
| | 4_1-1_mismatch_ | CCAGCAUGAACUAAUCAUC |
| | A-C | |
| | 25_6-6_internal_ | |
| | loop-symmetric_ | |
| | CCUAUA-GCAGUC | |
| 1346 | -2_6-6_internal_ | GAUAUUGUGACAUAUCUCUGCACUCAU |
| | loop-symmetric_ | CACACAAAAGAUGCAAAUCUUCAACAU |
| | GAGAUA-GCGAAG | AAUCCACAGGGAGGGGGCAUCUUAAU |
| | 5_1-1_mismatch_ | AGCGAAGUGAACUAAUCAUC |
| | A-C | |
| | 25_6-6_internal_ | |
| | loop-symmetric_ | |
| | CCUAUA-AACAUA | |
| 1347 | -2_6-6_internal_ | GAUAUUGUGACAUAUCUCUGCACUCAU |
| | loop-symmetric_ | CACACAAAAGAUGCAAAUCUUCGUAAA |
| | GAGAUA-CGGGGG | AAUCCACAGGGAGGGGGCAUUUUAAU |
| | 25_6-6_internal_ | ACGGGGGUGAACUAAUCAUC |
| | loop-symmetric_ | |
| | CCUAUA-GUAAAA | |
| 1348 | -2_6-6_internal_ | GAUAUUGUGACAUAUCUCUGCACUCAU |
| | loop-symmetric_ | CACACAAAAGAUGCAAAUCUUGAUAA |
| | GAGAUA-GUAUAA | UGAUCCACAGGGAGGGGGCAUUUCAA |
| | 3_1-1_mismatch_ | UAGUAUAAUGAACUAAUCAUC |
| | A-C | |
| | 26_6-6_internal_ | |
| | loop-symmetric_ | |
| | CUAUAG-GAUAAU | |
| 1349 | -2_6-6_internal_ | GAUAUUGUGACAUAUCUCUGCACUCAU |
| | loop-symmetric_ | CACACAAAAGAUGCAAAUCUUAGUCUU |
| | GAGAUA-ACGACA | GAUCCACAGGGAGGGGGCAUUCUAAU |
| | 4_1-1_mismatch_ | AACGACAUGAACUAAUCAUC |
| | A-C | |
| | 26_6-6_internal_ | |
| | loop-symmetric_ | |
| | CUAUAG-AGUCUU | |
| 1350 | -2_6-6_internal_ | GAUAUUGUGACAUAUCUCUGCACUCAU |
| | loop-symmetric_ | CACACAAAAGAUGCAAAUCUUGGCGCU |
| | GAGAUA-GCAAUA | GAUCCACAGGGAGGGGGCAUCUUAAU |
| | 5_1-1_mismatch_ | AGCAAUAUGAACUAAUCAUC |
| | A-C | |
| | 26_6-6_internal_ | |
| | loop-symmetric_ | |
| | CUAUAG-GGCGCU | |
| 1351 | -2_6-6_internal_ | GAUAUUGUGACAUAUCUCUGCACUCAU |
| | loop-symmetric_ | CACACAAAAGAUGCAAAUCUUGGCACU |
| | GAGAUA-GGAUAG | GAUCCACAGGGAGGGGGCAUUUUAAU |
| | 26_6-6_internal_ | AGGAUAGUGAACUAAUCAUC |
| | loop-symmetric_ | |
| | CUAUAG-GGCACU | |
| 1352 | -2_6-6_internal_ | GAUAUUGUGACAUAUCUCUGCACUCAU |
| | loop-symmetric_ | CACACAAAAGAUGCAAAUCUAUACUCG |
| | GAGAUA-AAAGUA | GAUCCACAGGGAGGGGGCAUUUCAAU |
| | 3_1-1_mismatch_ | AAAAGUAUGAACUAAUCAUC |
| | A-C | |
| | 27_6-6_internal_ | |
| | loop-symmetric_ | |
| | UAUAGA-AUACUC | |
| 1353 | -2_6-6_internal_ | GAUAUUGUGACAUAUCUCUGCACUCAU |
| | loop-symmetric_ | CACACAAAAGAUGCAAAUCUGAGCGUG |
| | GAGAUA-GUGGAA | GAUCCACAGGGAGGGGGCAUUCUAAU |
| | 4_1-1_mismatch_ | AGUGGAAUGAACUAAUCAUC |
| | A-C | |
| | 27_6-6_internal_ | |
| | loop-symmetric_ | |
| | UAUAGA-GAGCGU | |
| 1354 | -2_6-6_internal_ | GAUAUUGUGACAUAUCUCUGCACUCAU |
| | loop-symmetric_ | CACACAAAAGAUGCAAAUCUGUUCGCG |
| | GAGAUA-CAAUGG | GAUCCACAGGGAGGGGGCAUCUUAAU |
| | 5_1-1_mismatch_ | ACAAUGGUGAACUAAUCAUC |
| | A-C | |
| | 27_6-6_internal_ | |
| | loop-symmetric_ | |
| | UAUAGA-GUUCGC | |
| 1355 | -2_6-6_internal_ | GAUAUUGUGACAUAUCUCUGCACUCAU |
| | loop-symmetric_ | CACACAAAAGAUGCAAAUCUAACAUUG |
| | GAGAUA-CCCGAG | GAUCCACAGGGAGGGGGCAUUUUAAU |
| | 27_6-6_internal_ | ACCCGAGUGAACUAAUCAUC |
| | loop-symmetric_ | |
| | UAUAGA-AACAUU | |
| 1356 | -2_6-6_internal_ | GAUAUUGUGACAUAUCUCUGCACUCAU |
| | loop-symmetric_ | CACACAAAAGAUGCAAAUCGCACAAAG |
| | GAGAUA-GGGACA | GAUCCACAGGGAGGGGGCAUUCUAAU |
| | 4_1-1_mismatch_ | AGGGACAUGAACUAAUCAUC |
| | A-C | |
| | 28_6-6_internal_ | |
| | loop-symmetric_ | |
| | AUAGAA-GCACAA | |
| 1357 | -2_6-6_internal_ | GAUAUUGUGACAUAUCUCUGCACUCAU |
| | loop-symmetric_ | CACACAAAAGAUGCAAAUCAAGUUCAG |
| | GAGAUA-GUAGAA | GAUCCACAGGGAGGGGGCAUCUUAAU |
| | 5_1-1_mismatch_ | AGUAGAAUGAACUAAUCAUC |
| | A-C | |
| | 28_6-6_internal_ | |
| | loop-symmetric_ | |
| | AUAGAA-AAGUUC | |
| 1358 | -2_6-6_internal_ | GAUAUUGUGACAUAUCUCUGCACUCAU |
| | loop-symmetric_ | CACACAAAAGAUGCAAAUCCGACAAAG |
| | GAGAUA-GGGUUA | GAUCCACAGGGAGGGGGCAUUUUAAU |
| | 28_6-6_internal_ | AGGGUUAUGAACUAAUCAUC |
| | loop-symmetric_ | |
| | AUAGAA-CGACAA | |
| 1359 | -2_6-6_internal_ | GAUAUUGUGACAUAUCUCUGCACUCAU |
| | loop-symmetric_ | CACACAAAAGAUGCAAAUAUGUAUUA |
| | GAGAUA-GCUACA | GGAUCCACAGGGAGGGGGCAUUUCAA |
| | 3_1-1_mismatch_ | UAGCUACAUGAACUAAUCAUC |
| | A-C | |
| | 29_6-6_internal_ | |
| | loop-symmetric_ | |
| | UAGAAG-AUGUAU | |
| 1360 | -2_6-6_internal_ | GAUAUUGUGACAUAUCUCUGCACUCAU |
| | loop-symmetric_ | CACACAAAAGAUGCAAAUGAGGCCUAG |
| | GAGAUA-AGCUAG | GAUCCACAGGGAGGGGGCAUUCUAAU |
| | 4_1-1_mismatch_ | AAGCUAGUGAACUAAUCAUC |
| | A-C | |
| | 29_6-6_internal_ | |
| | loop-symmetric_ | |
| | UAGAAG-GAGGCC | |
| 1361 | -2_6-6_internal_ | GAUAUUGUGACAUAUCUCUGCACUCAU |
| | loop-symmetric_ | CACACAAAAGAUGCAAAUAAUGGCUA |
| | GAGAUA-CCAUAA | GGAUCCACAGGGAGGGGGCAUCUUAA |
| | 5_1-1_mismatch_ | UACCAUAAUGAACUAAUCAUC |
| | A-C | |
| | 29_6-6_internal_ | |
| | loop-symmetric_ | |
| | UAGAAG-AAUGGC | |
| 1362 | -2_6-6_internal_ | GAUAUUGUGACAUAUCUCUGCACUCAU |
| | loop-symmetric_ | CACACAAAAGAUGCAAAUACAAGUUA |
| | GAGAUA-CGCGAG | GGAUCCACAGGGAGGGGGCAUUUUAA |
| | 29_6-6_internal_ | UACGCGAGUGAACUAAUCAUC |
| | loop-symmetric_ | |
| | UAGAAG-ACAAGU | |
| 1363 | -2_6-6_internal_ | GAUAUUGUGACAUAUCUCUGCACUCAU |
| | loop-symmetric_ | CACACAAAAGAUGCAAAAAGCGAAUA |
| | GAGAUA-CAGGUA | GGAUCCACAGGGAGGGGGCAUUUCAA |
| | 3_1-1_mismatch_ | UACAGGUAUGAACUAAUCAUC |
| | A-C | |
| | 30_6-6_internal_ | |
| | loop-symmetric_ | |
| | AGAAGA-AAGCGA | |
| 1364 | -2_6-6_internal_ | GAUAUUGUGACAUAUCUCUGCACUCAU |
| | loop-symmetric_ | CACACAAAAGAUGCAAAGUAUUGAUA |
| | GAGAUA-GUGUGG | GGAUCCACAGGGAGGGGGCAUUCUAA |
| | 4_1-1_mismatch_ | UAGUGUGGUGAACUAAUCAUC |
| | A-C | |
| | 30_6-6_internal_ | |
| | loop-symmetric_ | |
| | AGAAGA-GUAUUG | |
| 1365 | -2_6-6_internal_ | GAUAUUGUGACAUAUCUCUGCACUCAU |
| | loop-symmetric_ | CACACAAAAGAUGCAAAAGGUACAUA |
| | GAGAUA-CAGAGA | GGAUCCACAGGGAGGGGGCAUCUUAA |
| | 5_1-1_mismatch_ | UACAGAGAUGAACUAAUCAUC |
| | A-C | |
| | 30_6-6_internal_ | |
| | loop-symmetric_ | |
| | AGAAGA-AGGUAC | |
| 1366 | -2_6-6_internal_ | GAUAUUGUGACAUAUCUCUGCACUCAU |
| | loop-symmetric_ | CACACAAAAGAUGCAAACGCCACAUAG |
| | GAGAUA-GAAUGA | GAUCCACAGGGAGGGGGCAUUUUAAU |
| | 30_6-6_internal_ | AGAAUGAUGAACUAAUCAUC |
| | loop-symmetric_ | |
| | AGAAGA-CGCCAC | |
| 1367 | -2_6-6_internal_ | GAUAUUGUGACAUAUCUCUGCACUCAU |
| | loop-symmetric_ | CACACAAAAGAUGCAACCGCAAUAUAG |
| | GAGAUA-ACUACA | GAUCCACAGGGAGGGGGCAUUUCAAU |
| | 3_1-1_mismatch_ | AACUACAUGAACUAAUCAUC |
| | A-C | |
| | 31_6-6_internal_ | |
| | loop-symmetric_ | |
| | GAAGAU-CCGCAA | |
| 1368 | -2_6-6_internal_ | GAUAUUGUGACAUAUCUCUGCACUCAU |
| | loop-symmetric_ | CACACAAAAGAUGCAACAAACGUAUAG |
| | GAGAUA-CACGCG | GAUCCACAGGGAGGGGGCAUUCUAAU |
| | 4_1-1_mismatch_ | ACACGCGUGAACUAAUCAUC |
| | A-C | |
| | 31_6-6_internal_ | |
| | loop-symmetric_ | |
| | GAAGAU-CAAACG | |
| 1369 | -2_6-6_internal_ | GAUAUUGUGACAUAUCUCUGCACUCAU |
| | loop-symmetric_ | CACACAAAAGAUGCAACUGCGGUAUAG |
| | GAGAUA-CCCGGG | GAUCCACAGGGAGGGGGCAUCUUAAU |
| | 5_1-1_mismatch_ | ACCCGGGUGAACUAAUCAUC |
| | A-C | |
| | 31_6-6_internal_ | |
| | loop-symmetric_ | |
| | GAAGAU-CUGCGG | |
| 1370 | -2_6-6_internal_ | GAUAUUGUGACAUAUCUCUGCACUCAU |
| | loop-symmetric_ | CACACAAAAGAUGCAACACACAUAUAG |
| | GAGAUA-GCGGGA | GAUCCACAGGGAGGGGGCAUUUUAAU |
| | 31_6-6_internal_ | AGCGGGAUGAACUAAUCAUC |
| | loop-symmetric_ | |
| | GAAGAU-CACACA | |
| 1371 | -2_6-6_internal_ | GAUAUUGUGACAUAUCUCUGCACUCAU |
| | loop-symmetric_ | CACACAAAAGAUGCAUUAGGGCUAUA |
| | GAGAUA-AUGAGG | GGAUCCACAGGGAGGGGGCAUUUCAA |
| | 3_1-1_mismatch_ | UAAUGAGGUGAACUAAUCAUC |
| | A-C | |
| | 32_6-6_internal_ | |
| | loop-symmetric_ | |
| | AAGAUU-UUAGGG | |
| 1372 | -2_6-6_internal_ | GAUAUUGUGACAUAUCUCUGCACUCAU |
| | loop-symmetric_ | CACACAAAAGAUGCAUGGUACCUAUAG |
| | GAGAUA-GCCAAA | GAUCCACAGGGAGGGGGCAUUCUAAU |
| | 4_1-1_mismatch_ | AGCCAAAUGAACUAAUCAUC |
| | A-C | |
| | 32_6-6_internal_ | |
| | loop-symmetric_ | |
| | AAGAUU-UGGUAC | |
| 1373 | -2_6-6_internal_ | GAUAUUGUGACAUAUCUCUGCACUCAU |
| | loop-symmetric_ | CACACAAAAGAUGCAUCAAAACUAUAG |
| | GAGAUA-CGAAAG | GAUCCACAGGGAGGGGGCAUCUUAAU |
| | 5_1-1_mismatch_ | ACGAAAGUGAACUAAUCAUC |
| | A-C | |
| | 32_6-6_internal_ | |
| | loop-symmetric_ | |
| | AAGAUU-UCAAAA | |
| 1374 | -2_6-6_internal_ | GAUAUUGUGACAUAUCUCUGCACUCAU |
| | loop-symmetric_ | CACACAAAAGAUGCACGACCCCUAUAG |
| | GAGAUA-CGAGGG | GAUCCACAGGGAGGGGGCAUUUUAAU |
| | 32_6-6_internal_ | ACGAGGGUGAACUAAUCAUC |
| | loop-symmetric_ | |
| | AAGAUU-CGACCC | |
| 1375 | -2_6-6_internal_ | GAUAUUGUGACAUAUCUCUGCACUCAU |
| | loop-symmetric_ | CACACAAAAGAUGCCGCAAAUCUAUAG |
| | GAGAUA-GUGGCA | GAUCCACAGGGAGGGGGCAUUUCAAU |
| | 3_1-1_mismatch_ | AGUGGCAUGAACUAAUCAUC |
| | A-C | |
| | 33_6-6_internal_ | |
| | loop-symmetric_ | |
| | AGAUUU-CGCAAA | |
| 1376 | -2_6-6_internal_ | GAUAUUGUGACAUAUCUCUGCACUCAU |
| | loop-symmetric_ | CACACAAAAGAUGCCCGGAGUCUAUAG |
| | GAGAUA-GACCAA | GAUCCACAGGGAGGGGGCAUUCUAAU |
| | 4_1-1_mismatch_ | AGACCAAUGAACUAAUCAUC |
| | A-C | |
| | 33_6-6_internal_ | |
| | loop-symmetric_ | |
| | AGAUUU-CCGGAG | |
| 1377 | -2_6-6_internal_ | GAUAUUGUGACAUAUCUCUGCACUCAU |
| | loop-symmetric_ | CACACAAAAGAUGCUCAAAAUCUAUAG |
| | GAGAUA-GGGGCA | GAUCCACAGGGAGGGGGCAUCUUAAU |
| | 5_1-1_mismatch_ | AGGGGCAUGAACUAAUCAUC |
| | A-C | |
| | 33_6-6_internal_ | |
| | loop-symmetric_ | |
| | AGAUUU-UCAAAA | |
| 1378 | -2_6-6_internal_ | GAUAUUGUGACAUAUCUCUGCACUCAU |
| | loop-symmetric_ | CACACAAAAGAUGCUACAUAUCUAUAG |
| | GAGAUA-GUAGCG | GAUCCACAGGGAGGGGGCAUUUUAAU |
| | 33_6-6_internal_ | AGUAGCGUGAACUAAUCAUC |
| | loop-symmetric_ | |
| | AGAUUU-UACAUA | |
| 1379 | -2_6-6_internal_ | GAUAUUGUGACAUAUCUCUGCACUCAU |
| | loop-symmetric_ | CACACAAAAGAUGGCCUAGUUCUAUAG |
| | GAGAUA-ACCCAA | GAUCCACAGGGAGGGGGCAUUUCAAU |
| | 3_1-1_mismatch_ | AACCCAAUGAACUAAUCAUC |
| | A-C | |
| | 34_6-6_internal_ | |
| | loop-symmetric_ | |
| | GAUUUG-GCCUAG | |
| 1380 | -2_6-6_internal_ | GAUAUUGUGACAUAUCUCUGCACUCAU |
| | loop-symmetric_ | CACACAAAAGAUGGUUUGAUUCUAUA |
| | GAGAUA-AAAGUA | GGAUCCACAGGGAGGGGGCAUUCUAA |
| | 4_1-1_mismatch_ | UAAAAGUAUGAACUAAUCAUC |
| | A-C | |
| | 34_6-6_internal_ | |
| | loop-symmetric_ | |
| | GAUUUG-GUUUGA | |
| 1381 | -2_6-6_internal_ | GAUAUUGUGACAUAUCUCUGCACUCAU |
| | loop-symmetric_ | CACACAAAAGAUGGUUACAUUCUAUA |
| | GAGAUA-ACAGCA | GGAUCCACAGGGAGGGGGCAUCUUAA |
| | 5_1-1_mismatch_ | UAACAGCAUGAACUAAUCAUC |
| | A-C | |
| | 34_6-6_internal_ | |
| | loop-symmetric_ | |
| | GAUUUG-GUUACA | |
| 1382 | -2_6-6_internal_ | GAUAUUGUGACAUAUCUCUGCACUCAU |
| | loop-symmetric_ | CACACAAAAGAUGAAUCAAUUCUAUA |
| | GAGAUA-ACCACG | GGAUCCACAGGGAGGGGGCAUUUUAA |
| | 34_6-6_internal_ | UAACCACGUGAACUAAUCAUC |
| | loop-symmetric_ | |
| | GAUUUG-AAUCAA | |
| 1383 | -2_6-6_internal_ | GAUAUUGUGACAUAUCUCUGCACUCAU |
| | loop-symmetric_ | CACACAAAAGAUUCCGUCCUUCUAUAG |
| | GAGAUA-AUGACA | GAUCCACAGGGAGGGGGCAUUUCAAU |
| | 3_1-1_mismatch_ | AAUGACAUGAACUAAUCAUC |
| | A-C | |
| | 35_6-6_internal_ | |
| | loop-symmetric_ | |
| | AUUUGC-UCCGUC | |
| 1384 | -2_6-6_internal_ | GAUAUUGUGACAUAUCUCUGCACUCAU |
| | loop-symmetric_ | CACACAAAAGAUAAGUGGCUUCUAUA |
| | GAGAUA-GCAAAA | GGAUCCACAGGGAGGGGGCAUUCUAA |
| | 4_1-1_mismatch_ | UAGCAAAAUGAACUAAUCAUC |
| | A-C | |
| | 35_6-6_internal_ | |
| | loop-symmetric_ | |
| | AUUUGC-AAGUGG | |
| 1385 | -2_6-6_internal_ | GAUAUUGUGACAUAUCUCUGCACUCAU |
| | loop-symmetric_ | CACACAAAAGAUAACGUCCUUCUAUAG |
| | GAGAUA-ACUGAG | GAUCCACAGGGAGGGGGCAUCUUAAU |
| | 5_1-1_mismatch_ | AACUGAGUGAACUAAUCAUC |
| | A-C | |
| | 35_6-6_internal_ | |
| | loop-symmetric_ | |
| | AUUUGC-AACGUC | |
| 1386 | -2_6-6_internal_ | GAUAUUGUGACAUAUCUCUGCACUCAU |
| | loop-symmetric_ | CACACAAAAGAUCUUCACCUUCUAUAG |
| | GAGAUA-CGGUGG | GAUCCACAGGGAGGGGGCAUUUUAAU |
| | 35_6-6_internal_ | ACGGUGGUGAACUAAUCAUC |
| | loop-symmetric_ | |
| | AUUUGC-CUUCAC | |
| 1387 | -2_6-6_internal_ | GAUAUUGUGACAUAUCUCUGCACUCAU |
| | loop-symmetric_ | CACACAAAAGACCGUUUUCUUCUAUAG |
| | GAGAUA-CGGAGA | GAUCCACAGGGAGGGGGCAUUUCAAU |
| | 3_1-1_mismatch_ | ACGGAGAUGAACUAAUCAUC |
| | A-C | |
| | 36_6-6_internal_ | |
| | loop-symmetric_ | |
| | UUUGCA-CCGUUU | |
| 1388 | -2_6-6_internal_ | GAUAUUGUGACAUAUCUCUGCACUCAU |
| | loop-symmetric_ | CACACAAAAGACUAGUCUCUUCUAUAG |
| | GAGAUA-GCUGGG | GAUCCACAGGGAGGGGGCAUUCUAAU |
| | 4_1-1_mismatch_ | AGCUGGGUGAACUAAUCAUC |
| | A-C | |
| | 36_6-6_internal_ | |
| | loop-symmetric_ | |
| | UUUGCA-CUAGUC | |
| 1389 | -2_6-6_internal_ | GAUAUUGUGACAUAUCUCUGCACUCAU |
| | loop-symmetric_ | CACACAAAAGAACACUUUCUUCUAUAG |
| | GAGAUA-GUGGGA | GAUCCACAGGGAGGGGGCAUCUUAAU |
| | 5_1-1_mismatch_ | AGUGGGAUGAACUAAUCAUC |
| | A-C | |
| | 36_6-6_internal_ | |
| | loop-symmetric_ | |
| | UUUGCA-ACACUU | |
| 1390 | -2_6-6_internal_ | GAUAUUGUGACAUAUCUCUGCACUCAU |
| | loop-symmetric_ | CACACAAAAGAGACCUUUCUUCUAUAG |
| | GAGAUA-GAAUAA | GAUCCACAGGGAGGGGGCAUUUUAAU |
| | 36_6-6_internal_ | AGAAUAAUGAACUAAUCAUC |
| | loop-symmetric_ | |
| | UUUGCA-GACCUU | |
| 1391 | -2_6-6_internal_ | GAUAUUGUGACAUAUCUCUGCACUCAU |
| | loop-symmetric_ | CACACAAAAGCACCCUAUCUUCUAUAG |
| | GAGAUA-GCGGAG | GAUCCACAGGGAGGGGGCAUUUCAAU |
| | 3_1-1_mismatch_ | AGCGGAGUGAACUAAUCAUC |
| | A-C | |
| | 37_6-6_internal_ | |
| | loop-symmetric_ | |
| | UUGCAU-CACCCU | |
| 1392 | -2_6-6_internal_ | GAUAUUGUGACAUAUCUCUGCACUCAU |
| | loop-symmetric_ | CACACAAAAGUCAUUUAUCUUCUAUAG |
| | GAGAUA-CCCUAG | GAUCCACAGGGAGGGGGCAUUCUAAU |
| | 4_1-1_mismatch_ | ACCCUAGUGAACUAAUCAUC |
| | A-C | |
| | 37_6-6_internal_ | |
| | loop-symmetric_ | |
| | UUGCAU-UCAUUU | |
| 1393 | -2_6-6_internal_ | GAUAUUGUGACAUAUCUCUGCACUCAU |
| | loop-symmetric_ | CACACAAAAGUUACCCAUCUUCUAUAG |
| | GAGAUA-GGCAGA | GAUCCACAGGGAGGGGGCAUUUUAAU |
| | 37_6-6_internal_ | AGGCAGAUGAACUAAUCAUC |
| | loop-symmetric_ | |
| | UUGCAU-UUACCC | |
| 1394 | -2_6-6_internal_ | GAUAUUGUGACAUAUCUCUGCACUCAU |
| | loop-symmetric_ | CACACAAAAUUUUUCAAUCUUCUAUAG |
| | GAGAUA-ACGAAA | GAUCCACAGGGAGGGGGCAUUUCAAU |
| | 3_1-1_mismatch_ | AACGAAAUGAACUAAUCAUC |
| | A-C | |
| | 38_6-6_internal_ | |
| | loop-symmetric_ | |
| | UGCAUC-UUUUUC | |
| 1395 | -2_6-6_internal_ | GAUAUUGUGACAUAUCUCUGCACUCAU |
| | loop-symmetric_ | CACACAAAAUCAUAUAAUCUUCUAUAG |
| | GAGAUA-AAGUGG | GAUCCACAGGGAGGGGGCAUUCUAAU |
| | 4_1-1_mismatch_ | AAAGUGGUGAACUAAUCAUC |
| | A-C | |
| | 38_6-6_internal_ | |
| | loop-symmetric_ | |
| | UGCAUC-UCAUAU | |
| 1396 | -2_6-6_internal_ | GAUAUUGUGACAUAUCUCUGCACUCAU |
| | loop-symmetric_ | CACACAAAAAUUAACAAUCUUCUAUAG |
| | GAGAUA-CAAUAG | GAUCCACAGGGAGGGGGCAUCUUAAU |
| | 5_1-1_mismatch_ | ACAAUAGUGAACUAAUCAUC |
| | A-C | |
| | 38_6-6_internal_ | |
| | loop-symmetric_ | |
| | UGCAUC-AUUAAC | |
| 1397 | -2_6-6_internal_ | GAUAUUGUGACAUAUCUCUGCACUCAU |
| | loop-symmetric_ | CACACAAAAAACAAUAAUCUUCUAUAG |
| | GAGAUA-CUGAGG | GAUCCACAGGGAGGGGGCAUUUUAAU |
| | 38_6-6_internal_ | ACUGAGGUGAACUAAUCAUC |
| | loop-symmetric_ | |
| | UGCAUC-AACAAU | |
| 1398 | -2_6-6_internal_ | GAUAUUGUGACAUAUCUCUGCACUCAU |
| | loop-symmetric_ | CACACAAAUACACAAAAUCUUCUAUAG |
| | GAGAUA-GCCGCA | GAUCCACAGGGAGGGGGCAUUUCAAU |
| | 3_1-1_mismatch_ | AGCCGCAUGAACUAAUCAUC |
| | A-C | |
| | 39_6-6_internal_ | |
| | loop-symmetric_ | |
| | GCAUCU-UACACA | |
| 1399 | -2_6-6_internal_ | GAUAUUGUGACAUAUCUCUGCACUCAU |
| | loop-symmetric_ | CACACAAAUUCCCGAAAUCUUCUAUAG |
| | GAGAUA-GAAGAA | GAUCCACAGGGAGGGGGCAUUCUAAU |
| | 4_1-1_mismatch_ | AGAAGAAUGAACUAAUCAUC |
| | A-C | |
| | 39_6-6_internal_ | |
| | loop-symmetric_ | |
| | GCAUCU-UUCCCG | |
| 1400 | -2_6-6_internal_ | GAUAUUGUGACAUAUCUCUGCACUCAU |
| | loop-symmetric_ | CACACAAACGCACGAAAUCUUCUAUAG |
| | GAGAUA-ACAGCG | GAUCCACAGGGAGGGGGCAUCUUAAU |
| | 5_1-1_mismatch_ | AACAGCGUGAACUAAUCAUC |
| | A-C | |
| | 39_6-6_internal_ | |
| | loop-symmetric_ | |
| | GCAUCU-CGCACG | |
| 1401 | -2_6-6_internal_ | GAUAUUGUGACAUAUCUCUGCACUCAU |
| | loop-symmetric_ | CACACAAUGAAUUCAAAUCUUCUAUAG |
| | GAGAUA-AAAAGG | GAUCCACAGGGAGGGGGCAUUUCAAU |
| | 3_1-1_mismatch_ | AAAAAGGUGAACUAAUCAUC |
| | A-C | |
| | 40_6-6_internal_ | |
| | loop-symmetric_ | |
| | CAUCUU-UGAAUU | |
| 1402 | -2_6-6_internal_ | GAUAUUGUGACAUAUCUCUGCACUCAU |
| | loop-symmetric_ | CACACAACGCUUUCAAAUCUUCUAUAG |
| | GAGAUA-CUAGCA | GAUCCACAGGGAGGGGGCAUUCUAAU |
| | 4_1-1_mismatch_ | ACUAGCAUGAACUAAUCAUC |
| | A-C | |
| | 40_6-6_internal_ | |
| | loop-symmetric_ | |
| | CAUCUU-CGCUUU | |
| 1403 | -2_6-6_internal_ | GAUAUUGUGACAUAUCUCUGCACUCAU |
| | loop-symmetric_ | CACACAAUGAAUACAAAUCUUCUAUAG |
| | GAGAUA-GACUGA | GAUCCACAGGGAGGGGGCAUCUUAAU |
| | 5_1-1_mismatch_ | AGACUGAUGAACUAAUCAUC |
| | A-C | |
| | 40_6-6_internal_ | |
| | loop-symmetric_ | |
| | CAUCUU-UGAAUA | |
| 1404 | -2_6-6_internal_ | GAUAUUGUGACAUAUCUCUGCACUCAU |
| | loop-symmetric_ | CACACAAUUAUUUCAAAUCUUCUAUAG |
| | GAGAUA-GUGAAA | GAUCCACAGGGAGGGGGCAUUUUAAU |
| | 40_6-6_internal_ | AGUGAAAUGAACUAAUCAUC |
| | loop-symmetric_ | |
| | CAUCUU-UUAUUU | |
| 1405 | -2_6-6_internal_ | GAUAUUGUGACAUAUCUCUGCACUCAU |
| | loop-symmetric_ | CACACACCUAGCGCAAAUCUUCUAUAG |
| | GAGAUA-GUGUGG | GAUCCACAGGGAGGGGGCAUUUCAAU |
| | 3_1-1_mismatch_ | AGUGUGGUGAACUAAUCAUC |
| | A-C | |
| | 41_6-6_internal_ | |
| | loop-symmetric_ | |
| | AUCUUU-CCUAGC | |
| 1406 | -2_6-6_internal_ | GAUAUUGUGACAUAUCUCUGCACUCAU |
| | loop-symmetric_ | CACACAUUUCUAGCAAAUCUUCUAUAG |
| | GAGAUA-CGAGGA | GAUCCACAGGGAGGGGGCAUUCUAAU |
| | 4_1-1_mismatch_ | ACGAGGAUGAACUAAUCAUC |
| | A-C | |
| | 41_6-6_internal_ | |
| | loop-symmetric_ | |
| | AUCUUU-UUUCUA | |
| 1407 | -2_6-6_internal_ | GAUAUUGUGACAUAUCUCUGCACUCAU |
| | loop-symmetric_ | CACACACGCAACGCAAAUCUUCUAUAG |
| | GAGAUA-CCGACA | GAUCCACAGGGAGGGGGCAUCUUAAU |
| | 5_1-1_mismatch_ | ACCGACAUGAACUAAUCAUC |
| | A-C | |
| | 41_6-6_internal_ | |
| | loop-symmetric_ | |
| | AUCUUU-CGCAAC | |
| 1408 | -2_6-6_internal_ | GAUAUUGUGACAUAUCUCUGCACUCAU |
| | loop-symmetric_ | CACACACUUCGGGCAAAUCUUCUAUAG |
| | GAGAUA-CUAGAG | GAUCCACAGGGAGGGGGCAUUUUAAU |
| | 41_6-6_internal_ | ACUAGAGUGAACUAAUCAUC |
| | loop-symmetric_ | |
| | AUCUUU-CUUCGG | |
| 1409 | -2_6-6_internal_ | GAUAUUGUGACAUAUCUCUGCACUCAU |
| | loop-symmetric_ | CACACCGCACUUGCAAAUCUUCUAUAG |
| | GAGAUA-CUGGGG | GAUCCACAGGGAGGGGGCAUUUCAAU |
| | 3_1-1_mismatch_ | ACUGGGGUGAACUAAUCAUC |
| | A-C | |
| | 42_6-6_internal_ | |
| | loop-symmetric_ | |
| | UCUUUU-CGCACU | |
| 1410 | -2_6-6_internal_ | GAUAUUGUGACAUAUCUCUGCACUCAU |
| | loop-symmetric_ | CACACCCCUACUGCAAAUCUUCUAUAG |
| | GAGAUA-GGAGGA | GAUCCACAGGGAGGGGGCAUUCUAAU |
| | 4_1-1_mismatch_ | AGGAGGAUGAACUAAUCAUC |
| | A-C | |
| | 42_6-6_internal_ | |
| | loop-symmetric_ | |
| | UCUUUU-CCCUAC | |
| 1411 | -2_6-6_internal_ | GAUAUUGUGACAUAUCUCUGCACUCAU |
| | loop-symmetric_ | CACACCCUCCCUGCAAAUCUUCUAUAG |
| | GAGAUA-AGCGGG | GAUCCACAGGGAGGGGGCAUCUUAAU |
| | 5_1-1_mismatch_ | AAGCGGGUGAACUAAUCAUC |
| | A-C | |
| | 42_6-6_internal_ | |
| | loop-symmetric_ | |
| | UCUUUU-CCUCCC | |
| 1412 | -2_6-6_internal_ | GAUAUUGUGACAUAUCUCUGCACUCAU |
| | loop-symmetric_ | CACACCUUCCCUGCAAAUCUUCUAUAG |
| | GAGAUA-GAAUGG | GAUCCACAGGGAGGGGGCAUUUUAAU |
| | 42_6-6_internal_ | AGAAUGGUGAACUAAUCAUC |
| | loop-symmetric_ | |
| | UCUUUU-CUUCCC | |
| 1413 | -2_6-6_internal_ | GAUAUUGUGACAUAUCUCUGCACUCAU |
| | loop-symmetric_ | CACAACUUCCAUGCAAAUCUUCUAUAG |
| | GAGAUA-AGGAGA | GAUCCACAGGGAGGGGGCAUUUCAAU |
| | 3_1-1_mismatch_ | AAGGAGAUGAACUAAUCAUC |
| | A-C | |
| | 43_6-6_internal_ | |
| | loop-symmetric_ | |
| | CUUUUG-ACUUCC | |
| 1414 | -2_6-6_internal_ | GAUAUUGUGACAUAUCUCUGCACUCAU |
| | loop-symmetric_ | CACAGCACCAAUGCAAAUCUUCUAUAG |
| | GAGAUA-CGGGUA | GAUCCACAGGGAGGGGGCAUUCUAAU |
| | 4_1-1_mismatch_ | ACGGGUAUGAACUAAUCAUC |
| | A-C | |
| | 43_6-6_internal_ | |
| | loop-symmetric_ | |
| | CUUUUG-GCACCA | |
| 1415 | -2_6-6_internal_ | GAUAUUGUGACAUAUCUCUGCACUCAU |
| | loop-symmetric_ | CACAACCCAUAUGCAAAUCUUCUAUAG |
| | GAGAUA-CAAGAG | GAUCCACAGGGAGGGGGCAUCUUAAU |
| | 5_1-1_mismatch_ | ACAAGAGUGAACUAAUCAUC |
| | A-C | |
| | 43_6-6_internal_ | |
| | loop-symmetric_ | |
| | CUUUUG-ACCCAU | |
| 1416 | -2_6-6_internal_ | GAUAUUGUGACAUAUCUCUGCACUCAU |
| | loop-symmetric_ | CACAGCUCUCAUGCAAAUCUUCUAUAG |
| | GAGAUA-CAGGCA | GAUCCACAGGGAGGGGGCAUUUUAAU |
| | 43_6-6_internal_ | ACAGGCAUGAACUAAUCAUC |
| | loop-symmetric_ | |
| | CUUUUG-GCUCUC | |
| 1417 | -2_6-6_internal_ | GAUAUUGUGACAUAUCUCUGCACUCAU |
| | loop-symmetric_ | CACUUCUUUGAUGCAAAUCUUCUAUAG |
| | GAGAUA-AGGGGA | GAUCCACAGGGAGGGGGCAUUUCAAU |
| | 3_1-1_mismatch_ | AAGGGGAUGAACUAAUCAUC |
| | A-C | |
| | 44_6-6_internal_ | |
| | loop-symmetric_ | |
| | UUUUGU-UUCUUU | |
| 1418 | -2_6-6_internal_ | GAUAUUGUGACAUAUCUCUGCACUCAU |
| | loop-symmetric_ | CACUCCACUGAUGCAAAUCUUCUAUAG |
| | GAGAUA-ACAACG | GAUCCACAGGGAGGGGGCAUUCUAAU |
| | 4_1-1_mismatch_ | AACAACGUGAACUAAUCAUC |
| | A-C | |
| | 44_6-6_internal_ | |
| | loop-symmetric_ | |
| | UUUUGU-UCCACU | |
| 1419 | -2_6-6_internal_ | GAUAUUGUGACAUAUCUCUGCACUCAU |
| | loop-symmetric_ | CACCUUUACGAUGCAAAUCUUCUAUAG |
| | GAGAUA-CACCCA | GAUCCACAGGGAGGGGGCAUCUUAAU |
| | 5_1-1_mismatch_ | ACACCCAUGAACUAAUCAUC |
| | A-C | |
| | 44_6-6_internal_ | |
| | loop-symmetric_ | |
| | UUUUGU-CUUUAC | |
| 1420 | -2_6-6_internal_ | GAUAUUGUGACAUAUCUCUGCACUCAU |
| | loop-symmetric_ | CACUAUAUCGAUGCAAAUCUUCUAUAG |
| | GAGAUA-CACUGA | GAUCCACAGGGAGGGGGCAUUUUAAU |
| | 44_6-6_internal_ | ACACUGAUGAACUAAUCAUC |
| | loop-symmetric_ | |
| | UUUUGU-UAUAUC | |
| 1421 | -6->0_7-7_internal_ | GAUAUUGUGACAUAUCUCUGCACUCAU |
| | loop-symmetric_ | CACACAAAAGAUGCAAAUCUUCUAUAG |
| | AGAUAUA- | GAUCCACAGGGAGGGGGCAUUUUAACC |
| | CCCUGGA | CUGGACUGAACUAAUCAUC |
| 1422 | -1_6-6_internal_ | GAUAUUGUGACAUAUCUCUGCACUCAU |
| | loop-symmetric_ | CACACAAAAGAUGCAAAUCUUCUAUAG |
| | AGAUAU-CCCAAG | GAUCCACAGGGAGGGGGCAUUUCAAUC |
| | 3_1-1_mismatch_ | CCAAGCUGAACUAAUCAUC |
| | A-C | |
| 1423 | -1_6-6_internal_ | GAUAUUGUGACAUAUCUCUGCACUCAU |
| | loop-symmetric_ | CACACAAAAGAUGCAAAUCUUCUAUAG |
| | AGAUAU-UCGCGG | GAUCCACAGGGAGGGGGCAUUCUAAU |
| | 4_1-1_mismatch_ | UCGCGGCUGAACUAAUCAUC |
| | A-C | |
| 1424 | -1_6-6_internal_ | GAUAUUGUGACAUAUCUCUGCACUCAU |
| | loop-symmetric_ | CACACAAAAGAUGCAAAUCUUCUAUAG |
| | AGAUAU-CCUGGA | GAUCCACAGGGAGGGGGCAUCUUAAUC |
| | 5_1-1_mismatch_ | CUGGACUGAACUAAUCAUC |
| | A-C | |
| 1425 | -1_6-6_internal_ | GAUAUUGUGACAUAUCUCUGCACUCAU |
| | loop-symmetric_ | CACACAAAAGAUGCAAAUCUUCUAUAG |
| | AGAUAU-UACUGA | GAUCCACAGGGAGGGGGCAUUUUAAU |
| | | UACUGACUGAACUAAUCAUC |
| 1426 | -6->0_7-7_internal_ | GAUAUUGUGACAUAUCUCUGCACUCAU |
| | loop-symmetric_ | CACACAAAAGAUGCAAAUCUUCACUUU |
| | AGAUAUA- | CAUCCACAGGGAGGGGGCAUUUUAACC |
| | CCCCUUG | CCUUGCUGAACUAAUCAUC |
| | 25_6-6_internal_ | |
| | loop-symmetric_ | |
| | CCUAUA-ACUUUC | |
| 1427 | -1_6-6_internal_ | GAUAUUGUGACAUAUCUCUGCACUCAU |
| | loop-symmetric_ | CACACAAAAGAUGCAAAUCUUCCUCCC |
| | AGAUAU-CGGACG | UAUCCACAGGGAGGGGGCAUUUCAAUC |
| | 3_1-1_mismatch_ | GGACGCUGAACUAAUCAUC |
| | A-C | |
| | 25_6-6_internal_ | |
| | loop-symmetric_ | |
| | CCUAUA-CUCCCU | |
| 1428 | -1_6-6_internal_ | GAUAUUGUGACAUAUCUCUGCACUCAU |
| | loop-symmetric_ | CACACAAAAGAUGCAAAUCUUCACAUA |
| | AGAUAU-UCUGAC | UAUCCACAGGGAGGGGGCAUUCUAAU |
| | 4_1-1_mismatch_ | UCUGACCUGAACUAAUCAUC |
| | A-C | |
| | 25_6-6_internal_ | |
| | loop-symmetric_ | |
| | CCUAUA-ACAUAU | |
| 1429 | -1_6-6_internal_ | GAUAUUGUGACAUAUCUCUGCACUCAU |
| | loop-symmetric_ | CACACAAAAGAUGCAAAUCUUCCAAUC |
| | AGAUAU-UAACAC | UAUCCACAGGGAGGGGGCAUCUUAAU |
| | 5_1-1_mismatch_ | UAACACCUGAACUAAUCAUC |
| | A-C | |
| | 25_6-6_internal_ | |
| | loop-symmetric_ | |
| | CCUAUA-CAAUCU | |
| 1430 | -1_6-6_internal_ | GAUAUUGUGACAUAUCUCUGCACUCAU |
| | loop-symmetric_ | CACACAAAAGAUGCAAAUCUUCGGCGC |
| | AGAUAU-CGCCGA | CAUCCACAGGGAGGGGGCAUUUUAAUC |
| | 25_6-6_internal_ | GCCGACUGAACUAAUCAUC |
| | loop-symmetric_ | |
| | CCUAUA-GGCGCC | |
| 1431 | -6->0_7-7_internal_ | GAUAUUGUGACAUAUCUCUGCACUCAU |
| | loop-symmetric_ | CACACAAAAGAUGCAAAUCUUGAGCUU |
| | AGAUAUA- | GAUCCACAGGGAGGGGGCAUUUUAAC |
| | CUUUAGG | UUUAGGCUGAACUAAUCAUC |
| | 26_6-6_internal_ | |
| | loop-symmetric_ | |
| | CUAUAG-GAGCUU | |
| 1432 | -1_6-6_internal_ | GAUAUUGUGACAUAUCUCUGCACUCAU |
| | loop-symmetric_ | CACACAAAAGAUGCAAAUCUUGGUCAU |
| | AGAUAU-CUGACA | GAUCCACAGGGAGGGGGCAUUUCAAUC |
| | 3_1-1_mismatch_ | UGACACUGAACUAAUCAUC |
| | A-C | |
| | 26_6-6_internal_ | |
| | loop-symmetric_ | |
| | CUAUAG-GGUCAU | |
| 1433 | -1_6-6_internal_ | GAUAUUGUGACAUAUCUCUGCACUCAU |
| | loop-symmetric_ | CACACAAAAGAUGCAAAUCUUAGCUCC |
| | AGAUAU-UACGGA | GAUCCACAGGGAGGGGGCAUUCUAAU |
| | 4_1-1_mismatch_ | UACGGACUGAACUAAUCAUC |
| | A-C | |
| | 26_6-6_internal_ | |
| | loop-symmetric_ | |
| | CUAUAG-AGCUCC | |
| 1434 | -1_6-6_internal_ | GAUAUUGUGACAUAUCUCUGCACUCAU |
| | loop-symmetric_ | CACACAAAAGAUGCAAAUCUUACGAUU |
| | AGAUAU-CCGGAC | GAUCCACAGGGAGGGGGCAUCUUAAUC |
| | 5_1-1_mismatch_ | CGGACCUGAACUAAUCAUC |
| | A-C | |
| | 26_6-6_internal_ | |
| | loop-symmetric_ | |
| | CUAUAG-ACGAUU | |
| 1435 | -1_6-6_internal_ | GAUAUUGUGACAUAUCUCUGCACUCAU |
| | loop-symmetric_ | CACACAAAAGAUGCAAAUCUUGACGCC |
| | AGAUAU-CGGCAG | GAUCCACAGGGAGGGGGCAUUUUAAU |
| | 26_6-6_internal_ | CGGCAGCUGAACUAAUCAUC |
| | loop-symmetric_ | |
| | CUAUAG-GACGCC | |
| 1436 | -6->0_7-7_internal_ | GAUAUUGUGACAUAUCUCUGCACUCAU |
| | loop-symmetric_ | CACACAAAAGAUGCAAAUCUAGCCCUG |
| | AGAUAUA- | GAUCCACAGGGAGGGGGCAUUUUAAC |
| | CGAGCGG | GAGCGGCUGAACUAAUCAUC |
| | 27_6-6_internal_ | |
| | loop-symmetric_ | |
| | UAUAGA-AGCCCU | |
| 1437 | -1_6-6_internal_ | GAUAUUGUGACAUAUCUCUGCACUCAU |
| | loop-symmetric_ | CACACAAAAGAUGCAAAUCUGAUUGCG |
| | AGAUAU-UUUAGG | GAUCCACAGGGAGGGGGCAUUUCAAU |
| | 3_1-1_mismatch_ | UUUAGGCUGAACUAAUCAUC |
| | A-C | |
| | 27_6-6_internal_ | |
| | loop-symmetric_ | |
| | UAUAGA-GAUUGC | |
| 1438 | -1_6-6_internal_ | GAUAUUGUGACAUAUCUCUGCACUCAU |
| | loop-symmetric_ | CACACAAAAGAUGCAAAUCUGGACCUG |
| | AGAUAU-CGCUAG | GAUCCACAGGGAGGGGGCAUUCUAAUC |
| | 4_1-1_mismatch_ | GCUAGCUGAACUAAUCAUC |
| | A-C | |
| | 27_6-6_internal_ | |
| | loop-symmetric_ | |
| | UAUAGA-GGACCU | |
| 1439 | -1_6-6_internal_ | GAUAUUGUGACAUAUCUCUGCACUCAU |
| | loop-symmetric_ | CACACAAAAGAUGCAAAUCUGGUUACG |
| | AGAUAU-UCCGGA | GAUCCACAGGGAGGGGGCAUCUUAAU |
| | 5_1-1_mismatch_ | UCCGGACUGAACUAAUCAUC |
| | A-C | |
| | 27_6-6_internal_ | |
| | loop-symmetric_ | |
| | UAUAGA-GGUUAC | |
| 1440 | -1_6-6_internal_ | GAUAUUGUGACAUAUCUCUGCACUCAU |
| | loop-symmetric_ | CACACAAAAGAUGCAAAUCUCAGUCUG |
| | AGAUAU-CAAACA | GAUCCACAGGGAGGGGGCAUUUUAAU |
| | 27_6-6_internal_ | CAAACACUGAACUAAUCAUC |
| | loop-symmetric_ | |
| | UAUAGA-CAGUCU | |
| 1441 | -6->0_7-7_internal_ | GAUAUUGUGACAUAUCUCUGCACUCAU |
| | loop-symmetric_ | CACACAAAAGAUGCAAAUCGCCAUGAG |
| | AGAUAUA- | GAUCCACAGGGAGGGGGCAUUUUAAC |
| | CUACCAC | UACCACCUGAACUAAUCAUC |
| | 28_6-6_internal_ | |
| | loop-symmetric_ | |
| | AUAGAA-GCCAUG | |
| 1442 | -1_6-6_internal_ | GAUAUUGUGACAUAUCUCUGCACUCAU |
| | loop-symmetric_ | CACACAAAAGAUGCAAAUCGCCAUGAG |
| | AGAUAU-UACUGG | GAUCCACAGGGAGGGGGCAUUUCAAU |
| | 3_1-1_mismatch_ | UACUGGCUGAACUAAUCAUC |
| | A-C | |
| | 28_6-6_internal_ | |
| | loop-symmetric_ | |
| | AUAGAA-GCCAUG | |
| 1443 | -1_6-6_internal_ | GAUAUUGUGACAUAUCUCUGCACUCAU |
| | loop-symmetric_ | CACACAAAAGAUGCAAAUCGUAGGAA |
| | AGAUAU-CUUGAC | GGAUCCACAGGGAGGGGGCAUUCUAA |
| | 4_1-1_mismatch_ | UCUUGACCUGAACUAAUCAUC |
| | A-C | |
| | 28_6-6_internal_ | |
| | loop-symmetric_ | |
| | AUAGAA-GUAGGA | |
| 1444 | -1_6-6_internal_ | GAUAUUGUGACAUAUCUCUGCACUCAU |
| | loop-symmetric_ | CACACAAAAGAUGCAAAUCAAGAACAG |
| | AGAUAU-UUUGAG | GAUCCACAGGGAGGGGGCAUCUUAAU |
| | 5_1-1_mismatch_ | UUUGAGCUGAACUAAUCAUC |
| | A-C | |
| | 28_6-6_internal_ | |
| | loop-symmetric_ | |
| | AUAGAA-AAGAAC | |
| 1445 | -1_6-6_internal_ | GAUAUUGUGACAUAUCUCUGCACUCAU |
| | loop-symmetric_ | CACACAAAAGAUGCAAAUCCCGGGAAG |
| | AGAUAU-CGGCGA | GAUCCACAGGGAGGGGGCAUUUUAAU |
| | 28_6-6_internal_ | CGGCGACUGAACUAAUCAUC |
| | loop-symmetric_ | |
| | AUAGAA-CCGGGA | |
| 1446 | -6->0_7-7_internal_ | GAUAUUGUGACAUAUCUCUGCACUCAU |
| | loop-symmetric_ | CACACAAAAGAUGCAAAUGCAGAUUA |
| | AGAUAUA- | GGAUCCACAGGGAGGGGGCAUUUUAA |
| | CCACCGA | CCACCGACUGAACUAAUCAUC |
| | 29_6-6_internal_ | |
| | loop-symmetric_ | |
| | UAGAAG-GCAGAU | |
| 1447 | -1_6-6_internal_ | GAUAUUGUGACAUAUCUCUGCACUCAU |
| | loop-symmetric_ | CACACAAAAGAUGCAAAUGCAGUCUAG |
| | AGAUAU-UGGCAG | GAUCCACAGGGAGGGGGCAUUUCAAU |
| | 3_1-1_mismatch_ | UGGCAGCUGAACUAAUCAUC |
| | A-C | |
| | 29_6-6_internal_ | |
| | loop-symmetric_ | |
| | UAGAAG-GCAGUC | |
| 1448 | -1_6-6_internal_ | GAUAUUGUGACAUAUCUCUGCACUCAU |
| | loop-symmetric_ | CACACAAAAGAUGCAAAUACAACCUAG |
| | AGAUAU-UAAAGA | GAUCCACAGGGAGGGGGCAUUCUAAU |
| | 4_1-1_mismatch_ | UAAAGACUGAACUAAUCAUC |
| | A-C | |
| | 29_6-6_internal_ | |
| | loop-symmetric_ | |
| | UAGAAG-ACAACC | |
| 1449 | -1_6-6_internal_ | GAUAUUGUGACAUAUCUCUGCACUCAU |
| | loop-symmetric_ | CACACAAAAGAUGCAAAUACAGAUUA |
| | AGAUAU-UCAAAC | GGAUCCACAGGGAGGGGGCAUCUUAA |
| | 5_1-1_mismatch_ | UUCAAACCUGAACUAAUCAUC |
| | A-C | |
| | 29_6-6_internal_ | |
| | loop-symmetric_ | |
| | UAGAAG-ACAGAU | |
| 1450 | -1_6-6_internal_ | GAUAUUGUGACAUAUCUCUGCACUCAU |
| | loop-symmetric_ | CACACAAAAGAUGCAAAUAUAGGUUA |
| | AGAUAU-UCGAGG | GGAUCCACAGGGAGGGGGCAUUUUAA |
| | 29_6-6_internal_ | UUCGAGGCUGAACUAAUCAUC |
| | loop-symmetric_ | |
| | UAGAAG-AUAGGU | |
| 1451 | -6->0_7-7_internal_ | GAUAUUGUGACAUAUCUCUGCACUCAU |
| | loop-symmetric_ | CACACAAAAGAUGCAAAAACCCCAUAG |
| | AGAUAUA- | GAUCCACAGGGAGGGGGCAUUUUAACC |
| | CCCUCAG | CUCAGCUGAACUAAUCAUC |
| | 30_6-6_internal_ | |
| | loop-symmetric_ | |
| | AGAAGA-AACCCC | |
| 1452 | -1_6-6_internal_ | GAUAUUGUGACAUAUCUCUGCACUCAU |
| | loop-symmetric_ | CACACAAAAGAUGCAAAGUCAAAAUA |
| | AGAUAU-CCAGAC | GGAUCCACAGGGAGGGGGCAUUUCAA |
| | 3_1-1_mismatch_ | UCCAGACCUGAACUAAUCAUC |
| | A-C | |
| | 30_6-6_internal_ | |
| | loop-symmetric_ | |
| | AGAAGA-GUCAAA | |
| 1453 | -1_6-6_internal_ | GAUAUUGUGACAUAUCUCUGCACUCAU |
| | loop-symmetric_ | CACACAAAAGAUGCAAAAUUAAAAUA |
| | AGAUAU-CAACGG | GGAUCCACAGGGAGGGGGCAUUCUAA |
| | 4_1-1_mismatch_ | UCAACGGCUGAACUAAUCAUC |
| | A-C | |
| | 30_6-6_internal_ | |
| | loop-symmetric_ | |
| | AGAAGA-AUUAAA | |
| 1454 | -1_6-6_internal_ | GAUAUUGUGACAUAUCUCUGCACUCAU |
| | loop-symmetric_ | CACACAAAAGAUGCAAACCCGUAAUAG |
| | AGAUAU-CACGAC | GAUCCACAGGGAGGGGGCAUCUUAAUC |
| | 5_1-1_mismatch_ | ACGACCUGAACUAAUCAUC |
| | A-C | |
| | 30_6-6_internal_ | |
| | loop-symmetric_ | |
| | AGAAGA-CCCGUA | |
| 1455 | -1_6-6_internal_ | GAUAUUGUGACAUAUCUCUGCACUCAU |
| | loop-symmetric_ | CACACAAAAGAUGCAAAAGAUAAAUA |
| | AGAUAU-UCAGAC | GGAUCCACAGGGAGGGGGCAUUUUAA |
| | 30_6-6_internal_ | UUCAGACCUGAACUAAUCAUC |
| | loop-symmetric_ | |
| | AGAAGA-AGAUAA | |
| 1456 | -6->0_7-7_internal_ | GAUAUUGUGACAUAUCUCUGCACUCAU |
| | loop-symmetric_ | CACACAAAAGAUGCAACGACAAUAUAG |
| | AGAUAUA- | GAUCCACAGGGAGGGGGCAUUUUAAC |
| | CACUUAA | ACUUAACUGAACUAAUCAUC |
| | 31_6-6_internal_ | |
| | loop-symmetric_ | |
| | GAAGAU-CGACAA | |
| 1457 | -1_6-6_internal_ | GAUAUUGUGACAUAUCUCUGCACUCAU |
| | loop-symmetric_ | CACACAAAAGAUGCAACGUUAAUAUA |
| | AGAUAU-CCAAAC | GGAUCCACAGGGAGGGGGCAUUUCAA |
| | 3_1-1_mismatch_ | UCCAAACCUGAACUAAUCAUC |
| | A-C | |
| | 31_6-6_internal_ | |
| | loop-symmetric_ | |
| | GAAGAU-CGUUAA | |
| 1458 | -1_6-6_internal_ | GAUAUUGUGACAUAUCUCUGCACUCAU |
| | loop-symmetric_ | CACACAAAAGAUGCAACAGUGGUAUA |
| | AGAUAU-UGCGAC | GGAUCCACAGGGAGGGGGCAUUCUAA |
| | 4_1-1_mismatch_ | UUGCGACCUGAACUAAUCAUC |
| | A-C | |
| | 31_6-6_internal_ | |
| | loop-symmetric_ | |
| | GAAGAU-CAGUGG | |
| 1459 | -1_6-6_internal_ | GAUAUUGUGACAUAUCUCUGCACUCAU |
| | loop-symmetric_ | CACACAAAAGAUGCAAUAGGGAUAUA |
| | AGAUAU-CAUUAA | GGAUCCACAGGGAGGGGGCAUCUUAA |
| | 5_1-1_mismatch_ | UCAUUAACUGAACUAAUCAUC |
| | A-C | |
| | 31_6-6_internal_ | |
| | loop-symmetric_ | |
| | GAAGAU-UAGGGA | |
| 1460 | -1_6-6_internal_ | GAUAUUGUGACAUAUCUCUGCACUCAU |
| | loop-symmetric_ | CACACAAAAGAUGCAACAGGAGUAUA |
| | AGAUAU-UGCCAA | GGAUCCACAGGGAGGGGGCAUUUUAA |
| | 31_6-6_internal_ | UUGCCAACUGAACUAAUCAUC |
| | loop-symmetric_ | |
| | GAAGAU-CAGGAG | |
| 1461 | -6->0_7-7_internal_ | GAUAUUGUGACAUAUCUCUGCACUCAU |
| | loop-symmetric_ | CACACAAAAGAUGCACUGGGCCUAUAG |
| | AGAUAUA- | GAUCCACAGGGAGGGGGCAUUUUAAC |
| | CAACCGG | AACCGGCUGAACUAAUCAUC |
| | 32_6-6_internal_ | |
| | loop-symmetric_ | |
| | AAGAUU-CUGGGC | |
| 1462 | -1_6-6_internal_ | GAUAUUGUGACAUAUCUCUGCACUCAU |
| | loop-symmetric_ | CACACAAAAGAUGCAUACGGGCUAUAG |
| | AGAUAU-UGUGGG | GAUCCACAGGGAGGGGGCAUUUCAAU |
| | 3_1-1_mismatch_ | UGUGGGCUGAACUAAUCAUC |
| | A-C | |
| | 32_6-6_internal_ | |
| | loop-symmetric_ | |
| | AAGAUU-UACGGG | |
| 1463 | -1_6-6_internal_ | GAUAUUGUGACAUAUCUCUGCACUCAU |
| | loop-symmetric_ | CACACAAAAGAUGCACAGGGGCUAUAG |
| | AGAUAU-UCCCUG | GAUCCACAGGGAGGGGGCAUUCUAAU |
| | 4_1-1_mismatch_ | UCCCUGCUGAACUAAUCAUC |
| | A-C | |
| | 32_6-6_internal_ | |
| | loop-symmetric_ | |
| | AAGAUU-CAGGGG | |
| 1464 | -1_6-6_internal_ | GAUAUUGUGACAUAUCUCUGCACUCAU |
| | loop-symmetric_ | CACACAAAAGAUGCAUUGAGACUAUA |
| | AGAUAU-UGGGAG | GGAUCCACAGGGAGGGGGCAUCUUAA |
| | 5_1-1_mismatch_ | UUGGGAGCUGAACUAAUCAUC |
| | A-C | |
| | 32_6-6_internal_ | |
| | loop-symmetric_ | |
| | AAGAUU-UUGAGA | |
| 1465 | -1_6-6_internal_ | GAUAUUGUGACAUAUCUCUGCACUCAU |
| | loop-symmetric_ | CACACAAAAGAUGCAUCGGCCCUAUAG |
| | AGAUAU-CAUUAC | GAUCCACAGGGAGGGGGCAUUUUAAU |
| | 32_6-6_internal_ | CAUUACCUGAACUAAUCAUC |
| | loop-symmetric_ | |
| | AAGAUU-UCGGCC | |
| 1466 | -6->0_7-7_internal_ | GAUAUUGUGACAUAUCUCUGCACUCAU |
| | loop-symmetric_ | CACACAAAAGAUGCUCAAGGUCUAUAG |
| | AGAUAUA- | GAUCCACAGGGAGGGGGCAUUUUAAC |
| | CUACCGG | UACCGGCUGAACUAAUCAUC |
| | 33_6-6_internal_ | |
| | loop-symmetric_ | |
| | AGAUUU-UCAAGG | |
| 1467 | -1_6-6_internal_ | GAUAUUGUGACAUAUCUCUGCACUCAU |
| | loop-symmetric_ | CACACAAAAGAUGCUUUCACUCUAUAG |
| | AGAUAU-UUUGGA | GAUCCACAGGGAGGGGGCAUUUCAAU |
| | 3_1-1_mismatch_ | UUUGGACUGAACUAAUCAUC |
| | A-C | |
| | 33_6-6_internal_ | |
| | loop-symmetric_ | |
| | AGAUUU-UUUCAC | |
| 1468 | -1_6-6_internal_ | GAUAUUGUGACAUAUCUCUGCACUCAU |
| | loop-symmetric_ | CACACAAAAGAUGCUUUGGCUCUAUAG |
| | AGAUAU-CUUGGA | GAUCCACAGGGAGGGGGCAUUCUAAUC |
| | 4_1-1_mismatch_ | UUGGACUGAACUAAUCAUC |
| | A-C | |
| | 33_6-6_internal_ | |
| | loop-symmetric_ | |
| | AGAUUU-UUUGGC | |
| 1469 | -1_6-6_internal_ | GAUAUUGUGACAUAUCUCUGCACUCAU |
| | loop-symmetric_ | CACACAAAAGAUGCCUCAGAUCUAUAG |
| | AGAUAU-CCAGGG | GAUCCACAGGGAGGGGGCAUCUUAAUC |
| | 5_1-1_mismatch_ | CAGGGCUGAACUAAUCAUC |
| | A-C | |
| | 33_6-6_internal_ | |
| | loop-symmetric_ | |
| | AGAUUU-CUCAGA | |
| 1470 | -1_6-6_internal_ | GAUAUUGUGACAUAUCUCUGCACUCAU |
| | loop-symmetric_ | CACACAAAAGAUGCCAUAAAUCUAUAG |
| | AGAUAU-CACUAG | GAUCCACAGGGAGGGGGCAUUUUAAU |
| | 33_6-6_internal_ | CACUAGCUGAACUAAUCAUC |
| | loop-symmetric_ | |
| | AGAUUU-CAUAAA | |
| 1471 | -6->0_7-7_internal_ | GAUAUUGUGACAUAUCUCUGCACUCAU |
| | loop-symmetric_ | CACACAAAAGAUGAGCCAGUUCUAUAG |
| | AGAUAUA- | GAUCCACAGGGAGGGGGCAUUUUAAC |
| | CUUUGUG | UUUGUGCUGAACUAAUCAUC |
| | 34_6-6_internal_ | |
| | loop-symmetric_ | |
| | GAUUUG-AGCCAG | |
| 1472 | -1_6-6_internal_ | GAUAUUGUGACAUAUCUCUGCACUCAU |
| | loop-symmetric_ | CACACAAAAGAUGAAACUAUUCUAUA |
| | AGAUAU-CAGGUG | GGAUCCACAGGGAGGGGGCAUUUCAA |
| | 3_1-1_mismatch_ | UCAGGUGCUGAACUAAUCAUC |
| | A-C | |
| | 34_6-6_internal_ | |
| | loop-symmetric_ | |
| | GAUUUG-AAACUA | |
| 1473 | -1_6-6_internal_ | GAUAUUGUGACAUAUCUCUGCACUCAU |
| | loop-symmetric_ | CACACAAAAGAUGGCUCAAUUCUAUAG |
| | AGAUAU-CACUGA | GAUCCACAGGGAGGGGGCAUUCUAAUC |
| | 4_1-1_mismatch_ | ACUGACUGAACUAAUCAUC |
| | A-C | |
| | 34_6-6_internal_ | |
| | loop-symmetric_ | |
| | GAUUUG-GCUCAA | |
| 1474 | -1_6-6_internal_ | GAUAUUGUGACAUAUCUCUGCACUCAU |
| | loop-symmetric_ | CACACAAAAGAUGGCGCCAUUCUAUAG |
| | AGAUAU-CACAAA | GAUCCACAGGGAGGGGGCAUCUUAAUC |
| | 5_1-1_mismatch_ | ACAAACUGAACUAAUCAUC |
| | A-C | |
| | 34_6-6_internal_ | |
| | loop-symmetric_ | |
| | GAUUUG-GCGCCA | |
| 1475 | -1_6-6_internal_ | GAUAUUGUGACAUAUCUCUGCACUCAU |
| | loop-symmetric_ | CACACAAAAGAUGAUUCGGUUCUAUA |
| | AGAUAU-UCUGGG | GGAUCCACAGGGAGGGGGCAUUUUAA |
| | 34_6-6_internal_ | UUCUGGGCUGAACUAAUCAUC |
| | loop-symmetric_ | |
| | GAUUUG-AUUCGG | |
| 1476 | -6->0_7-7_internal_ | GAUAUUGUGACAUAUCUCUGCACUCAU |
| | loop-symmetric_ | CACACAAAAGAUUACUGACUUCUAUAG |
| | AGAUAUA- | GAUCCACAGGGAGGGGGCAUUUUAAC |
| | CGAGCGG | GAGCGGCUGAACUAAUCAUC |
| | 35_6-6_internal_ | |
| | loop-symmetric_ | |
| | AUUUGC-UACUGA | |
| 1477 | -1_6-6_internal_ | GAUAUUGUGACAUAUCUCUGCACUCAU |
| | loop-symmetric_ | CACACAAAAGAUUAAUAACUUCUAUA |
| | AGAUAU-UACCAA | GGAUCCACAGGGAGGGGGCAUUUCAA |
| | 3_1-1_mismatch_ | UUACCAACUGAACUAAUCAUC |
| | A-C | |
| | 35_6-6_internal_ | |
| | loop-symmetric_ | |
| | AUUUGC-UAAUAA | |
| 1478 | -1_6-6_internal_ | GAUAUUGUGACAUAUCUCUGCACUCAU |
| | loop-symmetric_ | CACACAAAAGAUCUUCACCUUCUAUAG |
| | AGAUAU-UGCCCA | GAUCCACAGGGAGGGGGCAUUCUAAU |
| | 4_1-1_mismatch_ | UGCCCACUGAACUAAUCAUC |
| | A-C | |
| | 35_6-6_internal_ | |
| | loop-symmetric_ | |
| | AUUUGC-CUUCAC | |
| 1479 | -1_6-6_internal_ | GAUAUUGUGACAUAUCUCUGCACUCAU |
| | loop-symmetric_ | CACACAAAAGAUCUUGCCCUUCUAUAG |
| | AGAUAU-UACCGG | GAUCCACAGGGAGGGGGCAUCUUAAU |
| | 5_1-1_mismatch_ | UACCGGCUGAACUAAUCAUC |
| | A-C | |
| | 35_6-6_internal_ | |
| | loop-symmetric_ | |
| | AUUUGC-CUUGCC | |
| 1480 | -1_6-6_internal_ | GAUAUUGUGACAUAUCUCUGCACUCAU |
| | loop-symmetric_ | CACACAAAAGAUCUUUACCUUCUAUAG |
| | AGAUAU-CGAAAA | GAUCCACAGGGAGGGGGCAUUUUAAU |
| | 35_6-6_internal_ | CGAAAACUGAACUAAUCAUC |
| | loop-symmetric_ | |
| | AUUUGC-CUUUAC | |
| 1481 | -6->0_7-7_internal_ | GAUAUUGUGACAUAUCUCUGCACUCAU |
| | loop-symmetric_ | CACACAAAAGACAGUUCUCUUCUAUAG |
| | AGAUAUA- | GAUCCACAGGGAGGGGGCAUUUUAAC |
| | CACUUGG | ACUUGGCUGAACUAAUCAUC |
| | 36_6-6_internal_ | |
| | loop-symmetric_ | |
| | UUUGCA-CAGUUC | |
| 1482 | -1_6-6_internal_ | GAUAUUGUGACAUAUCUCUGCACUCAU |
| | loop-symmetric_ | CACACAAAAGACAAUCUUCUUCUAUAG |
| | AGAUAU-UGGCGA | GAUCCACAGGGAGGGGGCAUUUCAAU |
| | 3_1-1_mismatch_ | UGGCGACUGAACUAAUCAUC |
| | A-C | |
| | 36_6-6_internal_ | |
| | loop-symmetric_ | |
| | UUUGCA-CAAUCU | |
| 1483 | -1_6-6_internal_ | GAUAUUGUGACAUAUCUCUGCACUCAU |
| | loop-symmetric_ | CACACAAAAGACCCCCUUCUUCUAUAG |
| | AGAUAU-CGUUAA | GAUCCACAGGGAGGGGGCAUUCUAAUC |
| | 4_1-1_mismatch_ | GUUAACUGAACUAAUCAUC |
| | A-C | |
| | 36_6-6_internal_ | |
| | loop-symmetric_ | |
| | UUUGCA-CCCCCU | |
| 1484 | -1_6-6_internal_ | GAUAUUGUGACAUAUCUCUGCACUCAU |
| | loop-symmetric_ | CACACAAAAGAAAGGUUUCUUCUAUA |
| | AGAUAU-UGUGGG | GGAUCCACAGGGAGGGGGCAUCUUAA |
| | 5_1-1_mismatch_ | UUGUGGGCUGAACUAAUCAUC |
| | A-C | |
| | 36_6-6_internal_ | |
| | loop-symmetric_ | |
| | UUUGCA-AAGGUU | |
| 1485 | -1_6-6_internal_ | GAUAUUGUGACAUAUCUCUGCACUCAU |
| | loop-symmetric_ | CACACAAAAGAGACCACUCUUCUAUAG |
| | AGAUAU-UUUAAG | GAUCCACAGGGAGGGGGCAUUUUAAU |
| | 36_6-6_internal_ | UUUAAGCUGAACUAAUCAUC |
| | loop-symmetric_ | |
| | UUUGCA-GACCAC | |
| 1486 | -6->0_7-7_internal_ | GAUAUUGUGACAUAUCUCUGCACUCAU |
| | loop-symmetric_ | CACACAAAAGCCAGAUAUCUUCUAUAG |
| | AGAUAUA- | GAUCCACAGGGAGGGGGCAUUUUAAC |
| | CUGCUAG | UGCUAGCUGAACUAAUCAUC |
| | 37_6-6_internal_ | |
| | loop-symmetric_ | |
| | UUGCAU-CCAGAU | |
| 1487 | -1_6-6_internal_ | GAUAUUGUGACAUAUCUCUGCACUCAU |
| | loop-symmetric_ | CACACAAAAGUCUUUUAUCUUCUAUAG |
| | AGAUAU-UGCAGA | GAUCCACAGGGAGGGGGCAUUUCAAU |
| | 3_1-1_mismatch_ | UGCAGACUGAACUAAUCAUC |
| | A-C | |
| | 37_6-6_internal_ | |
| | loop-symmetric_ | |
| | UUGCAU-UCUUUU | |
| 1488 | -1_6-6_internal_ | GAUAUUGUGACAUAUCUCUGCACUCAU |
| | loop-symmetric_ | CACACAAAAGCUUUCUAUCUUCUAUAG |
| | AGAUAU-CGACGG | GAUCCACAGGGAGGGGGCAUUCUAAUC |
| | 4_1-1_mismatch_ | GACGGCUGAACUAAUCAUC |
| | A-C | |
| | 37_6-6_internal_ | |
| | loop-symmetric_ | |
| | UUGCAU-CUUUCU | |
| 1489 | -1_6-6_internal_ | GAUAUUGUGACAUAUCUCUGCACUCAU |
| | loop-symmetric_ | CACACAAAAGUCAUCUAUCUUCUAUAG |
| | AGAUAU-UAUAGG | GAUCCACAGGGAGGGGGCAUCUUAAU |
| | 5_1-1_mismatch_ | UAUAGGCUGAACUAAUCAUC |
| | A-C | |
| | 37_6-6_internal_ | |
| | loop-symmetric_ | |
| | UUGCAU-UCAUCU | |
| 1490 | -1_6-6_internal_ | GAUAUUGUGACAUAUCUCUGCACUCAU |
| | loop-symmetric_ | CACACAAAAGCAUAGUAUCUUCUAUAG |
| | AGAUAU-CACCUG | GAUCCACAGGGAGGGGGCAUUUUAAU |
| | 37_6-6_internal_ | CACCUGCUGAACUAAUCAUC |
| | loop-symmetric_ | |
| | UUGCAU-CAUAGU | |
| 1491 | -6->0_7-7_internal_ | GAUAUUGUGACAUAUCUCUGCACUCAU |
| | loop-symmetric_ | CACACAAAACAGAAUAAUCUUCUAUAG |
| | AGAUAUA- | GAUCCACAGGGAGGGGGCAUUUUAAC |
| | CUCUAAG | UCUAAGCUGAACUAAUCAUC |
| | 38_6-6_internal_ | |
| | loop-symmetric_ | |
| | UGCAUC-CAGAAU | |
| 1492 | -1_6-6_internal_ | GAUAUUGUGACAUAUCUCUGCACUCAU |
| | loop-symmetric_ | CACACAAAAAUUUACAAUCUUCUAUAG |
| | AGAUAU-UCGAGA | GAUCCACAGGGAGGGGGCAUUUCAAU |
| | 3_1-1_mismatch_ | UCGAGACUGAACUAAUCAUC |
| | A-C | |
| | 38_6-6_internal_ | |
| | loop-symmetric_ | |
| | UGCAUC-AUUUAC | |
| 1493 | -1_6-6_internal_ | GAUAUUGUGACAUAUCUCUGCACUCAU |
| | loop-symmetric_ | CACACAAAACCUAUCAAUCUUCUAUAG |
| | AGAUAU-CUCCGG | GAUCCACAGGGAGGGGGCAUUCUAAUC |
| | 4_1-1_mismatch_ | UCCGGCUGAACUAAUCAUC |
| | A-C | |
| | 38_6-6_internal_ | |
| | loop-symmetric_ | |
| | UGCAUC-CCUAUC | |
| 1494 | -1_6-6_internal_ | GAUAUUGUGACAUAUCUCUGCACUCAU |
| | loop-symmetric_ | CACACAAAAUCUCCCAAUCUUCUAUAG |
| | AGAUAU-UGGACG | GAUCCACAGGGAGGGGGCAUUUUAAU |
| | 38_6-6_internal_ | UGGACGCUGAACUAAUCAUC |
| | loop-symmetric_ | |
| | UGCAUC-UCUCCC | |
| 1495 | -6->0_7-7_internal_ | GAUAUUGUGACAUAUCUCUGCACUCAU |
| | loop-symmetric_ | CACACAAAUUCUAAAAAUCUUCUAUAG |
| | AGAUAUA- | GAUCCACAGGGAGGGGGCAUUUUAACC |
| | CCGUUAA | GUUAACUGAACUAAUCAUC |
| | 39_6-6_internal_ | |
| | loop-symmetric_ | |
| | GCAUCU-UUCUAA | |
| 1496 | -1_6-6_internal_ | GAUAUUGUGACAUAUCUCUGCACUCAU |
| | loop-symmetric_ | CACACAAACUCUAAAAAUCUUCUAUAG |
| | AGAUAU-CAUUGG | GAUCCACAGGGAGGGGGCAUUUCAAUC |
| | 3_1-1_mismatch_ | AUUGGCUGAACUAAUCAUC |
| | A-C | |
| | 39_6-6_internal_ | |
| | loop-symmetric_ | |
| | GCAUCU-CUCUAA | |
| 1497 | -1_6-6_internal_ | GAUAUUGUGACAUAUCUCUGCACUCAU |
| | loop-symmetric_ | CACACAAAUACGAGAAAUCUUCUAUAG |
| | AGAUAU-UGACCA | GAUCCACAGGGAGGGGGCAUUCUAAU |
| | 4_1-1_mismatch_ | UGACCACUGAACUAAUCAUC |
| | A-C | |
| | 39_6-6_internal_ | |
| | loop-symmetric_ | |
| | GCAUCU-UACGAG | |
| 1498 | -1_6-6_internal_ | GAUAUUGUGACAUAUCUCUGCACUCAU |
| | loop-symmetric_ | CACACAAAUCCGGAAAAUCUUCUAUAG |
| | AGAUAU-UCGGGG | GAUCCACAGGGAGGGGGCAUCUUAAU |
| | 5_1-1_mismatch_ | UCGGGGCUGAACUAAUCAUC |
| | A-C | |
| | 39_6-6_internal_ | |
| | loop-symmetric_ | |
| | GCAUCU-UCCGGA | |
| 1499 | -1_6-6_internal_ | GAUAUUGUGACAUAUCUCUGCACUCAU |
| | loop-symmetric_ | CACACAAAUUAACAAAAUCUUCUAUAG |
| | AGAUAU-UCCUGG | GAUCCACAGGGAGGGGGCAUUUUAAU |
| | 39_6-6_internal_ | UCCUGGCUGAACUAAUCAUC |
| | loop-symmetric_ | |
| | GCAUCU-UUAACA | |
| 1500 | -6->0_7-7_internal_ | GAUAUUGUGACAUAUCUCUGCACUCAU |
| | loop-symmetric_ | CACACAAUAUAGCCAAAUCUUCUAUAG |
| | AGAUAUA- | GAUCCACAGGGAGGGGGCAUUUUAAC |
| | CACAGGA | ACAGGACUGAACUAAUCAUC |
| | 40_6-6_internal_ | |
| | loop-symmetric_ | |
| | CAUCUU-UAUAGC | |
| 1501 | -1_6-6_internal_ | GAUAUUGUGACAUAUCUCUGCACUCAU |
| | loop-symmetric_ | CACACAAUGCAGCCAAAUCUUCUAUAG |
| | AGAUAU-CAGGUA | GAUCCACAGGGAGGGGGCAUUUCAAUC |
| | 3_1-1_mismatch_ | AGGUACUGAACUAAUCAUC |
| | A-C | |
| | 40_6-6_internal_ | |
| | loop-symmetric_ | |
| | CAUCUU-UGCAGC | |
| 1502 | -1_6-6_internal_ | GAUAUUGUGACAUAUCUCUGCACUCAU |
| | loop-symmetric_ | CACACAACCACAACAAAUCUUCUAUAG |
| | AGAUAU-UAGGGA | GAUCCACAGGGAGGGGGCAUUCUAAU |
| | 4_1-1_mismatch_ | UAGGGACUGAACUAAUCAUC |
| | A-C | |
| | 40_6-6_internal_ | |
| | loop-symmetric_ | |
| | CAUCUU-CCACAA | |
| 1503 | -1_6-6_internal_ | GAUAUUGUGACAUAUCUCUGCACUCAU |
| | loop-symmetric_ | CACACAAUUUACUCAAAUCUUCUAUAG |
| | AGAUAU-CUCCGA | GAUCCACAGGGAGGGGGCAUCUUAAUC |
| | 5_1-1_mismatch_ | UCCGACUGAACUAAUCAUC |
| | A-C | |
| | 40_6-6_internal_ | |
| | loop-symmetric_ | |
| | CAUCUU-UUUACU | |
| 1504 | -1_6-6_internal_ | GAUAUUGUGACAUAUCUCUGCACUCAU |
| | loop-symmetric_ | CACACAACACUUACAAAUCUUCUAUAG |
| | AGAUAU-UCCAUG | GAUCCACAGGGAGGGGGCAUUUUAAU |
| | 40_6-6_internal_ | UCCAUGCUGAACUAAUCAUC |
| | loop-symmetric_ | |
| | CAUCUU-CACUUA | |
| 1505 | -6->0_7-7_internal_ | GAUAUUGUGACAUAUCUCUGCACUCAU |
| | loop-symmetric_ | CACACAUAUGCGGCAAAUCUUCUAUAG |
| | AGAUAUA- | GAUCCACAGGGAGGGGGCAUUUUAACC |
| | CCUUAGA | UUAGACUGAACUAAUCAUC |
| | 41_6-6_internal_ | |
| | loop-symmetric_ | |
| | AUCUUU-UAUGCG | |
| 1506 | -1_6-6_internal_ | GAUAUUGUGACAUAUCUCUGCACUCAU |
| | loop-symmetric_ | CACACAUACAAAGCAAAUCUUCUAUAG |
| | AGAUAU-CCCUGA | GAUCCACAGGGAGGGGGCAUUUCAAUC |
| | 3_1-1_mismatch_ | CCUGACUGAACUAAUCAUC |
| | A-C | |
| | 41_6-6_internal_ | |
| | loop-symmetric_ | |
| | AUCUUU-UACAAA | |
| 1507 | -1_6-6_internal_ | GAUAUUGUGACAUAUCUCUGCACUCAU |
| | loop-symmetric_ | CACACACUUAACGCAAAUCUUCUAUAG |
| | AGAUAU-CCAGGG | GAUCCACAGGGAGGGGGCAUUCUAAUC |
| | 4_1-1_mismatch_ | CAGGGCUGAACUAAUCAUC |
| | A-C | |
| | 41_6-6_internal_ | |
| | loop-symmetric_ | |
| | AUCUUU-CUUAAC | |
| 1508 | -1_6-6_internal_ | GAUAUUGUGACAUAUCUCUGCACUCAU |
| | loop-symmetric_ | CACACACGCCCGGCAAAUCUUCUAUAG |
| | AGAUAU-CACCAG | GAUCCACAGGGAGGGGGCAUCUUAAUC |
| | 5_1-1_mismatch_ | ACCAGCUGAACUAAUCAUC |
| | A-C | |
| | 41_6-6_internal_ | |
| | loop-symmetric_ | |
| | AUCUUU-CGCCCG | |
| 1509 | -1_6-6_internal_ | GAUAUUGUGACAUAUCUCUGCACUCAU |
| | loop-symmetric_ | CACACAUUUUCCGCAAAUCUUCUAUAG |
| | AGAUAU-CGAGUA | GAUCCACAGGGAGGGGGCAUUUUAAU |
| | 41_6-6_internal_ | CGAGUACUGAACUAAUCAUC |
| | loop-symmetric_ | |
| | AUCUUU-UUUUCC | |
| 1510 | -6->0_7-7_internal_ | GAUAUUGUGACAUAUCUCUGCACUCAU |
| | loop-symmetric_ | CACACUUCACUUGCAAAUCUUCUAUAG |
| | AGAUAUA- | GAUCCACAGGGAGGGGGCAUUUUAACC |
| | CCCUCCA | CUCCACUGAACUAAUCAUC |
| | 42_6-6_internal_ | |
| | loop-symmetric_ | |
| | UCUUUU-UUCACU | |
| 1511 | -1_6-6_internal_ | GAUAUUGUGACAUAUCUCUGCACUCAU |
| | loop-symmetric_ | CACACUUCGCUUGCAAAUCUUCUAUAG |
| | AGAUAU-CGUUAC | GAUCCACAGGGAGGGGGCAUUUCAAUC |
| | 3_1-1_mismatch_ | GUUACCUGAACUAAUCAUC |
| | A-C | |
| | 42_6-6_internal_ | |
| | loop-symmetric_ | |
| | UCUUUU-UUCGCU | |
| 1512 | -1_6-6_internal_ | GAUAUUGUGACAUAUCUCUGCACUCAU |
| | loop-symmetric_ | CACACUAUAACUGCAAAUCUUCUAUAG |
| | AGAUAU-UCUGGA | GAUCCACAGGGAGGGGGCAUUCUAAU |
| | 4_1-1_mismatch_ | UCUGGACUGAACUAAUCAUC |
| | A-C | |
| | 42_6-6_internal_ | |
| | loop-symmetric_ | |
| | UCUUUU-UAUAAC | |
| 1513 | -1_6-6_internal_ | GAUAUUGUGACAUAUCUCUGCACUCAU |
| | loop-symmetric_ | CACACCUUGUCUGCAAAUCUUCUAUAG |
| | AGAUAU-UAGGGA | GAUCCACAGGGAGGGGGCAUCUUAAU |
| | 5_1-1_mismatch_ | UAGGGACUGAACUAAUCAUC |
| | A-C | |
| | 42_6-6_internal_ | |
| | loop-symmetric_ | |
| | UCUUUU-CUUGUC | |
| 1514 | -1_6-6_internal_ | GAUAUUGUGACAUAUCUCUGCACUCAU |
| | loop-symmetric_ | CACACUCUCGUUGCAAAUCUUCUAUAG |
| | AGAUAU-UAAGUG | GAUCCACAGGGAGGGGGCAUUUUAAU |
| | 42_6-6_internal_ | UAAGUGCUGAACUAAUCAUC |
| | loop-symmetric_ | |
| | UCUUUU-UCUCGU | |
| 1515 | -6->0_7-7_internal_ | GAUAUUGUGACAUAUCUCUGCACUCAU |
| | loop-symmetric_ | CACAGUGCUAAUGCAAAUCUUCUAUAG |
| | AGAUAUA- | GAUCCACAGGGAGGGGGCAUUUUAAC |
| | CACGCGG | ACGCGGCUGAACUAAUCAUC |
| | 43_6-6_internal_ | |
| | loop-symmetric_ | |
| | CUUUUG-GUGCUA | |
| 1516 | -1_6-6_internal_ | GAUAUUGUGACAUAUCUCUGCACUCAU |
| | loop-symmetric_ | CACAAGCUAUAUGCAAAUCUUCUAUAG |
| | AGAUAU-CCAAAC | GAUCCACAGGGAGGGGGCAUUUCAAUC |
| | 3_1-1_mismatch_ | CAAACCUGAACUAAUCAUC |
| | A-C | |
| | 43_6-6_internal_ | |
| | loop-symmetric_ | |
| | CUUUUG-AGCUAU | |
| 1517 | -1_6-6_internal_ | GAUAUUGUGACAUAUCUCUGCACUCAU |
| | loop-symmetric_ | CACAGCCCCCAUGCAAAUCUUCUAUAG |
| | AGAUAU-CCGACG | GAUCCACAGGGAGGGGGCAUUCUAAUC |
| | 4_1-1_mismatch_ | CGACGCUGAACUAAUCAUC |
| | A-C | |
| | 43_6-6_internal_ | |
| | loop-symmetric_ | |
| | CUUUUG-GCCCCC | |
| 1518 | -1_6-6_internal_ | GAUAUUGUGACAUAUCUCUGCACUCAU |
| | loop-symmetric_ | CACAGUGCUCAUGCAAAUCUUCUAUAG |
| | AGAUAU-UGAAAC | GAUCCACAGGGAGGGGGCAUCUUAAU |
| | 5_1-1_mismatch_ | UGAAACCUGAACUAAUCAUC |
| | A-C | |
| | 43_6-6_internal_ | |
| | loop-symmetric_ | |
| | CUUUUG-GUGCUC | |
| 1519 | -1_6-6_internal_ | GAUAUUGUGACAUAUCUCUGCACUCAU |
| | loop-symmetric_ | CACAAUUUGUAUGCAAAUCUUCUAUA |
| | AGAUAU-CCUAGA | GGAUCCACAGGGAGGGGGCAUUUUAA |
| | 43_6-6_internal_ | UCCUAGACUGAACUAAUCAUC |
| | loop-symmetric_ | |
| | CUUUUG-AUUUGU | |
| 1520 | -6->0_7-7_internal_ | GAUAUUGUGACAUAUCUCUGCACUCAU |
| | loop-symmetric_ | CACUUUACUGAUGCAAAUCUUCUAUAG |
| | AGAUAUA- | GAUCCACAGGGAGGGGGCAUUUUAAC |
| | CUCUUAA | UCUUAACUGAACUAAUCAUC |
| | 44_6-6_internal_ | |
| | loop-symmetric_ | |
| | UUUUGU-UUUACU | |
| 1521 | -1_6-6_internal_ | GAUAUUGUGACAUAUCUCUGCACUCAU |
| | loop-symmetric_ | CACUUUAGUGAUGCAAAUCUUCUAUA |
| | AGAUAU-UGCGUA | GGAUCCACAGGGAGGGGGCAUUUCAA |
| | 3_1-1_mismatch_ | UUGCGUACUGAACUAAUCAUC |
| | A-C | |
| | 44_6-6_internal_ | |
| | loop-symmetric_ | |
| | UUUUGU-UUUAGU | |
| 1522 | -1_6-6_internal_ | GAUAUUGUGACAUAUCUCUGCACUCAU |
| | loop-symmetric_ | CACUAGCUCGAUGCAAAUCUUCUAUAG |
| | AGAUAU-CUUUAC | GAUCCACAGGGAGGGGGCAUUCUAAUC |
| | 4_1-1_mismatch_ | UUUACCUGAACUAAUCAUC |
| | A-C | |
| | 44_6-6_internal_ | |
| | loop-symmetric_ | |
| | UUUUGU-UAGCUC | |
| 1523 | -1_6-6_internal_ | GAUAUUGUGACAUAUCUCUGCACUCAU |
| | loop-symmetric_ | CACCUUUCCGAUGCAAAUCUUCUAUAG |
| | AGAUAU-UUUAGA | GAUCCACAGGGAGGGGGCAUUUUAAU |
| | 44_6-6_internal_ | UUUAGACUGAACUAAUCAUC |
| | loop-symmetric_ | |
| | UUUUGU-CUUUCC | |
| 1524 | -11_2-2_bulge- | GAUAUUGUGAAUUAUCUCUGGCCUCA |
| | symmetric_ | UCACUAAAAAGAUGACAAUCUUCUUA |
| | GU-UG | AGGAUCCAACGGGAGGGGAGAUUUUA |
| | -1_2-2_bulge- | AUCCAUCUCUGAUGUAAUCAUC |
| | symmetric_ | |
| | AU-CC | |
| | 8_2-2_bulge- | |
| | symmetric_ | |
| | GC-AG | |
| | 18_2-2_bulge- | |
| | symmetric_ | |
| | UG-AC | |
| | 28_2-2_bulge- | |
| | symmetric_ | |
| | AU-UA | |
| | 38_2-2_bulge- | |
| | symmetric_ | |
| | UG-AC | |
| | 48_2-2_bulge- | |
| | symmetric_ | |
| | GU-UA | |
| | 58_2-2_bulge- | |
| | symmetric_ | |
| | UG-GC | |
| | 68_2-2_bulge- | |
| | symmetric_ | |
| | UG-AU | |
| 1525 | -10_3-3_bulge- | GAUAUUGUGACAACGCUCUGCACGAUU |
| | symmetric_ | CACACAACAAAUGCAAAUAUGCUAUAG |
| | GUU-CCG | GAAGAACAGGGAGAAAGCAUUUUAUU |
| | -1->1_3-3_bulge- | UUAUCUCUGCCGUAAUCAUC |
| | symmetric_ | |
| | UAU-UUU | |
| | 10_3-3_bulge- | |
| | symmetric_ | |
| | CCC-AAA | |
| | 21_3-3_bulge- | |
| | symmetric_ | |
| | GGA-AGA | |
| | 32_3-3_bulge- | |
| | symmetric_ | |
| | AAG-AUG | |
| | 43_3-3_bulge- | |
| | symmetric_ | |
| | CUU-CAA | |
| | 54_3-3_bulge- | |
| | symmetric_ | |
| | UGA-GAU | |
| | 65_3-3_bulge- | |
| | symmetric_ | |
| | AUA-ACG | |
| 1526 | -9_4-4_bulge- | GAUAAUACGACAUAUCGAACCACUCAU |
| | symmetric_ | CUAUGAAAAGAUGAACCUCUUCUAUCA |
| | GUUC-CUUG | |
| | 0->3_4-4_bulge- | |
| | symmetric_ | |
| | AUUA-CUUC | |
| | 12_4-4_bulge- | |
| | symmetric_ | |
| | CCUC-CCUU | |
| | 24_4-4_bulge- | CCUCCACAGGCCUUGGGCAUUUCUUCA |
| | symmetric_ | UAUCUCUCUUGUAAUCAUC |
| | UCCU-CACC | |
| | 36_4-4_bulge- | |
| | symmetric_ | |
| | UUUG-AACC | |
| | 48_4-4_bulge- | |
| | symmetric_ | |
| | GUGU-UAUG | |
| | 60_4-4_bulge- | |
| | symmetric_ | |
| | CAGA-GAAC | |
| | 72_4-4_bulge- | |
| | symmetric_ | |
| | ACAA-AUAC | |
| 1527 | -8_5-5_internal_ | GAUAUUGUGCGAAUUCUCUGCAGAUU |
| | loop-symmetric_ | CCACACAAACAAGCCAAAUCUUGAUUC |
| | GUUCA-AUUCG | GGAUCCACCACAUGGGGGCAUACUUCU |
| | 1_5-5_internal_ | AUAUCUCAUUCGUAAUCAUC |
| | loop-symmetric_ | |
| | UUAAA-ACUUC | |
| | 14_5-5_internal_ | |
| | loop-symmetric_ | |
| | UCCCU-CACAU | |
| | 27_5-5_internal_ | |
| | loop-symmetric_ | |
| | UAUAG-GAUUC | |
| | 40_5-5_internal_ | |
| | loop-symmetric_ | |
| | CAUCU-CAAGC | |
| | 53_5-5_internal_ | |
| | loop-symmetric_ | |
| | AUGAG-GAUUC | |
| | 66_5-5_internal_ | |
| | loop-symmetric_ | |
| | UAUGU-CGAAU | |
| 1528 | -7_6-6_internal_ | GACCUGGGGACAUAUCGAGAGUCUCAU |
| | loop-symmetric_ | CACCUCUAUGAUGCAAAACACGGAUAG |
| | GUUCAG-AUAAUG | GAUCAUCUCAGAGGGGGCUUUAACAU |
| | 2_6-6_internal_ | AUAUCUAUAAUGUAAUCAUC |
| | loop-symmetric_ | |
| | UAAAAU-UUUAAC | |
| | 16_6-6_internal_ | |
| | loop-symmetric_ | |
| | CCUGUG-AUCUCA | |
| | 30_6-6_internal_ | |
| | loop-symmetric_ | |
| | AGAAGA-ACACGG | |
| | 44_6-6_internal_ | |
| | loop-symmetric_ | |
| | UUUUGU-CUCUAU | |
| | 58_6-6_internal_ | |
| | loop-symmetric_ | |
| | UGCAGA-GAGAGU | |
| | 72_6-6_internal_ | |
| | loop-symmetric_ | |
| | ACAAUA-CCUGGG | |
| 1529 | -6_7-7_internal_ | GAUAUUGUGAAGCUGGACUGCACUCCA |
| | loop-symmetric_ | AAGUGAAAAGAUGGUACUACUCUAUA |
| | GUUCAGA- | GGUAAUUGUGGGAGGGGAUAAUACAA |
| | GGUCAUG | UAUAUCGGUCAUGUAAUCAUC |
| | 3_7-7_internal_ | |
| | loop-symmetric_ | |
| | AAAAUGC- | |
| | AUAAUAC | |
| | 18_7-7_internal_ | |
| | loop-symmetric_ | |
| | UGUGGAU- | |
| | UAAUUGU | |
| | 33_7-7_internal_ | |
| | loop-symmetric_ | |
| | AGAUUUG- | |
| | GUACUAC | |
| | 48_7-7_internal_ | |
| | loop-symmetric_ | |
| | GUGUGAU- | |
| | CAAAGUG | |
| | 63_7-7_internal_ | |
| | loop-symmetric_ | |
| | AGAUAUG- | |
| | AGCUGGA | |
| 1530 | -5_8-8_internal_ | GAUACUAUUAAUUAUCUCUGGUGGAU |
| | loop-symmetric_ | GGACACAAAAACUCCGUCUCUUCUAUC |
| | GUUCAGAG- | UAAUAGUCAGGGAGGCGAUUUAGUAA |
| | GCGUCCAA | UAUAUGCGUCCAAUAAUCAUC |
| | 4_8-8_internal_ | |
| | loop-symmetric_ | |
| | AAAUGCCC- | |
| | CGAUUUAG | |
| | 20_8-8_internal_ | |
| | loop-symmetric_ | |
| | UGGAUCCU- | |
| | CUAAUAGU | |
| | 36_8-8_internal_ | |
| | loop-symmetric_ | |
| | UUUGCAUC- | |
| | ACUCCGUC | |
| | 52_8-8_internal_ | |
| | loop-symmetric_ | |
| | GAUGAGUG- | |
| | GUGGAUGG | |
| | 68_8-8_internal_ | |
| | loop-symmetric_ | |
| | UGUCACAA- | |
| | CUAUUAAU | |
| 1531 | -9_2-2_bulge- | GAUACGGUGACAUAUCCGUGCACUCAU |
| | symmetric_ | CCAACAAAAGAUGGUAAUCUUCUAUUC |
| | UC-AC | GAUCCACAGGUUGGGGGCAUUUGUAU |
| | 2_2-2_bulge- | AUAUCUCUACACUAAUCAUC |
| | symmetric_ | |
| | UA-GU | |
| | 14_2-2_bulge- | |
| | symmetric_ | |
| | UC-UU | |
| | 26_2-2_bulge- | |
| | symmetric_ | |
| | CU-UC | |
| | 38_2-2_bulge- | |
| | symmetric_ | |
| | UG-GU | |
| | 50_2-2_bulge- | |
| | symmetric_ | |
| | GU-CA | |
| | 62_2-2_bulge- | |
| | symmetric_ | |
| | GA-CG | |
| | 74_2-2_bulge- | |
| | symmetric_ | |
| | AA-CG | |
| 1532 | -8_3-3_bulge- | GAUAUUGUGUGUUAUCUCUGCAGAAA |
| | symmetric_ | UCACACAAACCUUGCAAAUCUUAGCUA |
| | UCA-AAU | GGAUCCACUAUGAGGGGGCAUACCAAU |
| | 3_3-3_bulge- | AUAUCUCAAUACUAAUCAUC |
| | symmetric_ | |
| | AAA-ACC | |
| | 16_3-3_bulge- | |
| | symmetric_ | |
| | CCU-UAU | |
| | 29_3-3_bulge- | |
| | symmetric_ | |
| | UAG-AGC | |
| | 42_3-3_bulge- | |
| | symmetric_ | |
| | UCU-CCU | |
| | 55_3-3_bulge- | |
| | symmetric_ | |
| | GAG-GAA | |
| | 68_3-3_bulge- | |
| | symmetric_ | |
| | UGU-UGU | |
| 1533 | -7_4-4_bulge- | GAGUACGUGACAUAUCCACCCACUCAU |
| | symmetric_ | CACCUCCAAGAUGCAAAGUAACUAUAG |
| | UCAG-GAUC | GAUCAUCUGGGAGGGGGCUGAAUAAU |
| | 4_4-4_bulge- | AUAUCUGAUCACUAAUCAUC |
| | symmetric_ | |
| | AAAU-UGAA | |
| | 18_4-4_bulge- | |
| | symmetric_ | |
| | UGUG-AUCU | |
| | 32_4-4_bulge- | |
| | symmetric_ | |
| | AAGA-GUAA | |
| | 46_4-4_bulge- | |
| | symmetric_ | |
| | UUGU-CUCC | |
| | 60_4-4_bulge- | |
| | symmetric_ | |
| | CAGA-CACC | |
| | 74_4-4_bulge- | |
| | symmetric_ | |
| | AAUA-GUAC | |
| 1534 | -6_5-5_internal_ | GAUAUUGUGAGUACACUCUGCACUCCC |
| | loop-symmetric_ | AAAACAAAAGAUGGUCACCUUCUAUA |
| | UCAGA-AACCU | GGCUAGUCAGGGAGGGGUAAAAUUAA |
| | 5_5-5_internal_ | UAUAUCAACCUACUAAUCAUC |
| | loop-symmetric_ | |
| | AAUGC-UAAAA | |
| | 20_5-5_internal_ | |
| | loop-symmetric_ | |
| | UGGAU-CUAGU | |
| | 35_5-5_internal_ | |
| | loop-symmetric_ | |
| | AUUUG-GUCAC | |
| | 50_5-5_internal_ | |
| | loop-symmetric_ | |
| | GUGAU-CCAAA | |
| | 65_5-5_internal_ | |
| | loop-symmetric_ | |
| | AUAUG-GUACA | |
| 1535 | -5_6-6_internal_ | GAUACUUUAUCAUAUCUCUGGUUGGC |
| | loop-symmetric_ | UCACACAAAAUACAGUAAUCUUCUAUC |
| | UCAGAG-AAGCAC | CUACGCACAGGGAGGCCUACCUUUAAU |
| | 6_6-6_internal_ | AUAUAAGCACACUAAUCAUC |
| | loop-symmetric_ | |
| | AUGCCC-CCUACC | |
| | 22_6-6_internal_ | |
| | loop-symmetric_ | |
| | GAUCCU-CCUACG | |
| | 38_6-6_internal_ | |
| | loop-symmetric_ | |
| | UGCAUC-UACAGU | |
| | 54_6-6_internal_ | |
| | loop-symmetric_ | |
| | UGAGUG-GUUGGC | |
| | 70_6-6_internal_ | |
| | loop-symmetric_ | |
| | UCACAA-CUUUAU | |
| 1536 | -4_7-7_internal_ | GAUAUUGUGACAUAUGCAUAAUCUCA |
| | loop-symmetric_ | UCACACUUCAUUCGCAAAUCUUCGCGA |
| | UCAGAGA- | UGCUCCACAGGGAUUUCCCUUUUUAAU |
| | CACCCCC | AUACACCCCCACUAAUCAUC |
| | 7_7-7_internal_ | |
| | loop-symmetric_ | |
| | UGCCCCC- | |
| | UUUCCCU | |
| | 24_7-7_internal_ | |
| | loop-symmetric_ | |
| | UCCUAUA- | |
| | GCGAUGC | |
| | 41_7-7_internal_ | |
| | loop-symmetric_ | |
| | AUCUUUU- | |
| | UUCAUUC | |
| | 58_7-7_internal_ | |
| | loop-symmetric_ | |
| | UGCAGAG- | |
| | GCAUAAU | |
| 1537 | -3_8-8_internal_ | GAUAUUGUGAGCUGCAAAUGCACUCA |
| | loop-symmetric_ | UCUGUGACCCGAUGCAAAUCCUACACA |
| | UCAGAGAU- | AGAUCCACAGGCAUUGCUGAUUUUAA |
| | UUGGGCGU | UAUUUGGGCGUACUAAUCAUC |
| | 8_8-8_internal_ | |
| | loop-symmetric_ | |
| | GCCCCCUC- | |
| | CAUUGCUG | |
| | 26_8-8_internal_ | |
| | loop-symmetric_ | |
| | CUAUAGAA- | |
| | CUACACAA | |
| | 44_8-8_internal_ | |
| | loop-symmetric_ | |
| | UUUUGUGU- | |
| | UGUGACCC | |
| | 62_8-8_internal_ | |
| | loop-symmetric_ | |
| | GAGAUAUG- | |
| | GCUGCAAA | |
| 1538 | -7_2-2_bulge- | GACCUUGUGACAUAUCGAUGCACUCAU |
| | symmetric_ | CACCGAAAAGAUGCAAACAUUCUAUAG |
| | AG-GA | GAUCAUCAGGGAGGGGGCUCUUUAAU |
| | 6_2-2_bulge- | AUAUCUGAGAACUAAUCAUC |
| | symmetric_ | |
| | AU-UC | |
| | 20_2-2_bulge- | |
| | symmetric_ | |
| | UG-AU | |
| | 34_2-2_bulge- | |
| | symmetric_ | |
| | GA-CA | |
| | 48_2-2_bulge- | |
| | symmetric_ | |
| | GU-CG | |
| | 62_2-2_bulge- | |
| | symmetric_ | |
| | GA-GA | |
| | 76_2-2_bulge- | |
| | symmetric_ | |
| | UA-CC | |
| 1539 | -6_3-3_bulge- | GAUAUUGUGAACCAUCUCUGCACUCCG |
| | symmetric_ | GACACAAAAGAUGGUUAUCUUCUAUA |
| | AGA-CAG | GGUAGCACAGGGAGGGGAACUUUUAA |
| | 7_3-3_bulge- | UAUAUCCAGGAACUAAUCAUC |
| | symmetric_ | |
| | UGC-AAC | |
| | 22_3-3_bulge- | |
| | symmetric_ | |
| | GAU-UAG | |
| | 37_3-3_bulge- | |
| | symmetric_ | |
| | UUG-GUU | |
| | 52_3-3_bulge- | |
| | symmetric_ | |
| | GAU-CGG | |
| | 67_3-3_bulge- | |
| | symmetric_ | |
| | AUG-ACC | |
| 1540 | -5_4-4_bulge- | GAUAACAAGACAUAUCUCUGGUAGCA |
| | symmetric_ | UCACACAAAACUCCCAAAUCUUCUAUC |
| | AGAG-AUAA | |
| | 8_4-4_bulge- | |
| | symmetric_ | |
| | GCCC-CUCG | |
| | 24_4-4_bulge- | CCUUCCACAGGGAGGCUCGAUUUUAAU |
| | symmetric_ | AUAUAUAAGAACUAAUCAUC |
| | UCCU-CCCU | |
| | 40_4-4_bulge- | |
| | symmetric_ | |
| | CAUC-CUCC | |
| | 56_4-4_bulge- | |
| | symmetric_ | |
| | AGUG-GUAG | |
| | 72_4-4_bulge- | |
| | symmetric_ | |
| | ACAA-ACAA | |
| 1541 | -4_5-5_internal_ | GAUAUUGUGACAUAUGAAUUCACUCA |
| | loop-symmetric_ | UCACACUUUAAAUGCAAAUCUUCACUC |
| | AGAGA-CAGGC | UGAUCCACAGGGAUCACUCAUUUUAAU |
| | 9_5-5_internal_ | AUACAGGCGAACUAAUCAUC |
| | loop-symmetric_ | |
| | CCCCC-UCACU | |
| | 26_5-5_internal_ | |
| | loop-symmetric_ | |
| | CUAUA-ACUCU | |
| | 43_5-5_internal_ | |
| | loop-symmetric_ | |
| | CUUUU-UUUAA | |
| | 60_5-5_internal_ | |
| | loop-symmetric_ | |
| | CAGAG-GAAUU | |
| 1542 | -3_6-6_internal_ | GAUAUUGUGAGUUCCGUCUGCACUCAU |
| | loop-symmetric_ | CUGAAAUAAGAUGCAAAUCGCAGAGA |
| | AGAGAU-CAGCCC | GGAUCCACAGGCCCACAGCAUUUUAAU |
| | 10_6-6_internal_ | AUCAGCCCGAACUAAUCAUC |
| | loop-symmetric_ | |
| | CCCCUC-CCCACA | |
| | 28_6-6_internal_ | |
| | loop-symmetric_ | |
| | AUAGAA-GCAGAG | |
| | 46_6-6_internal_ | |
| | loop-symmetric_ | |
| | UUGUGU-UGAAAU | |
| | 64_6-6_internal_ | |
| | loop-symmetric_ | |
| | GAUAUG-GUUCCG | |
| 1543 | -2_7-7_internal_ | GAUAUCCGAAGUUAUCUCUGCACUACC |
| | loop-symmetric_ | CCCUCAAAAGAUGCAACAAAUGGAUAG |
| | AGAGAUA- | GAUCCACACCUCGACGGCAUUUUAAUA |
| | AAGGCCG | AAGGCCGGAACUAAUCAUC |
| | 11_7-7_internal_ | |
| | loop-symmetric_ | |
| | CCCUCCC- | |
| | CCUCGAC | |
| | 30_7-7_internal_ | |
| | loop-symmetric_ | |
| | AGAAGAU- | |
| | CAAAUGG | |
| | 49_7-7_internal_ | |
| | loop-symmetric_ | |
| | UGUGAUG- | |
| | ACCCCCU | |
| | 68_7-7_internal_ | |
| | loop-symmetric_ | |
| | UGUCACA- | |
| | CCGAAGU | |
| 1544 | -1_8-8_internal_ | GAUAUUGUGACAUAUCUCUGAAGGGU |
| | loop-symmetric_ | AGACACAAAAGAUGACCUGUACCUAUA |
| | AGAGAUAU- | GGAUCCAGUAUACAAGGGCAUUUUAA |
| | UGUGAUAA | UUGUGAUAAGAACUAAUCAUC |
| | 12_8-8_internal_ | |
| | loop-symmetric_ | |
| | CCUCCCUG- | |
| | GUAUACAA | |
| | 32_8-8_internal_ | |
| | loop-symmetric_ | |
| | AAGAUUUG- | |
| | ACCUGUAC | |
| | 52_8-8_internal_ | |
| | loop-symmetric_ | |
| | GAUGAGUG- | |
| | AAGGGUAG | |
| 1545 | -5_2-2_bulge- | GAUAAGGUGACAUAUCUCUGGUCUCA |
| | symmetric_ | UCACACAAAACCUGCAAAUCUUCUAUU |
| | AG-GC | UGAUCCACAGGGAGGAUGCAUUUUAA |
| | 10_2-2_bulge- | UAUAUGCCUGAACUAAUCAUC |
| | symmetric_ | |
| | CC-AU | |
| | 26_2-2_bulge- | |
| | symmetric_ | |
| | CU-UU | |
| | 42_2-2_bulge- | |
| | symmetric_ | |
| | UC-CC | |
| | 58_2-2_bulge- | |
| | symmetric_ | |
| | UG-GU | |
| | 74_2-2_bulge- | |
| | symmetric_ | |
| | AA-AG | |
| 1546 | -4_3-3_bulge- | GAUAUUGUGACAUAUAUAUGCACUCA |
| | symmetric_ | UCACACCUUAGAUGCAAAUCUUCCCAA |
| | AGA-GGA | GGAUCCACAGGGAUAAGGCAUUUUAA |
| | 11_3-3_bulge- | UAUAGGACUGAACUAAUCAUC |
| | symmetric_ | |
| | CCC-UAA | |
| | 28_3-3_bulge- | |
| | symmetric_ | |
| | AUA-CCA | |
| | 45_3-3_bulge- | |
| | symmetric_ | |
| | UUU-CUU | |
| | 62_3-3_bulge- | |
| | symmetric_ | |
| | GAG-AUA | |
| 1547 | -3_4-4_bulge- | GAUAUUGUGAGCCUUCUCUGCACUCAU |
| | symmetric_ | CUGUGAAAAGAUGCAAAUCCGGCAUA |
| | AGAU-UAGG | GGAUCCACAGGCACCGGGCAUUUUAAU |
| | 12_4-4_bulge- | AUUAGGCUGAACUAAUCAUC |
| | symmetric_ | |
| | CCUC-CACC | |
| | 30_4-4_bulge- | |
| | symmetric_ | |
| | AGAA-CGGC | |
| | 48_4-4_bulge- | |
| | symmetric_ | |
| | GUGU-UGUG | |
| | 66_4-4_bulge- | |
| | symmetric_ | |
| | UAUG-GCCU | |
| 1548 | -2_5-5_internal_ | GAUAUGCUUCCAUAUCUCUGCACUAUA |
| | loop-symmetric_ | GUCACAAAAGAUGCAAUAAUCCUAUA |
| | AGAUA-AUGAC | GGAUCCACAUUACCGGGGCAUUUUAAU |
| | 13_5-5_internal_ | AAUGACCUGAACUAAUCAUC |
| | loop-symmetric_ | |
| | CUCCC-UUACC | |
| | 32_5-5_internal_ | |
| | loop-symmetric_ | |
| | AAGAU-UAAUC | |
| | 51_5-5_internal_ | |
| | loop-symmetric_ | |
| | UGAUG-AUAGU | |
| | 70_5-5_internal_ | |
| | loop-symmetric_ | |
| | UCACA-GCUUC | |
| 1549 | -1_6-6_internal_ | GAUAUUGUGACAUAUCUCUGAUGUGU |
| | loop-symmetric_ | UCACACAAAAGAUGAACUCGUUCUAUA |
| | AGAUAU-UAAGAC | GGAUCCAAUUACUGGGGGCAUUUUAA |
| | 14_6-6_internal_ | UUAAGACCUGAACUAAUCAUC |
| | loop-symmetric_ | |
| | UCCCUG-AUUACU | |
| | 34_6-6_internal_ | |
| | loop-symmetric_ | |
| | GAUUUG-AACUCG | |
| | 54_6-6_internal_ | |
| | loop-symmetric_ | |
| | UGAGUG-AUGUGU | |
| 1550 | -6->0_7-7_ | GAUAUUGUGACAUAUCGGUCGAAUCA |
| | internal_ | UCACACAAAAGCGAGUUUUCUUCUAUA |
| | loop-symmetric_ | GGAUCAUUAAAAAGGGGGCAUUUUAA |
| | AGAUAUA- | GAUUUACCUGAACUAAUCAUC |
| | GAUUUAC | |
| | 15_7-7_internal_ | |
| | loop-symmetric_ | |
| | CCCUGUG- | |
| | AUUAAAA | |
| | 36_7-7_internal_ | |
| | loop-symmetric_ | |
| | UUUGCAU- | |
| | CGAGUUU | |
| | 57_7-7_internal_ | |
| | loop-symmetric_ | |
| | GUGCAGA- | |
| | GGUCGAA | |
| 1551 | -6->1_8-8_ | GAUAUUGUGACAACCACGACCACUCAU |
| | internal_ | CACACAAUAAAAACUAAUCUUCUAUAG |
| | loop-symmetric_ | GAAAGUAUCAGAGGGGGCAUUUUAUG |
| | AGAUAUAU- | CGCUUGCUGAACUAAUCAUC |
| | UGCGCUUG | |
| | 16_8-8_internal_ | |
| | loop-symmetric_ | |
| | CCUGUGGA- | |
| | AAGUAUCA | |
| | 38_8-8_internal_ | |
| | loop-symmetric_ | |
| | UGCAUCUU- | |
| | UAAAAACU | |
| | 60_8-8_internal_ | |
| | loop-symmetric_ | |
| | CAGAGAUA- | |
| | ACCACGAC | |
| 1552 | -3_2-2_bulge- | GAUAUUGUGAGCUAUCUCUGCACUCAU |
| | symmetric_ | CCGACAAAAGAUGCAAAUCGGCUAUAG |
| | AU-CA | GAUCCACAGGUUGGGGGCAUUUUAAU |
| | 14_2-2_bulge- | AUCACUCUGAACUAAUCAUC |
| | symmetric_ | |
| | UC-UU | |
| | 32_2-2_bulge- | |
| | symmetric_ | |
| | AA-GG | |
| | 50_2-2_bulge- | |
| | symmetric_ | |
| | GU-CG | |
| | 68_2-2_bulge- | |
| | symmetric_ | |
| | UG-GC | |
| 1553 | -2_3-3_bulge- | GAUAUCCGGACAUAUCUCUGCACUGUG |
| | symmetric_ | CACACAAAAGAUGCAAUGGUUCUAUA |
| | AUA-GCC | GGAUCCACAACCAGGGGGCAUUUUAAU |
| | 15_3-3_bulge- | AGCCCUCUGAACUAAUCAUC |
| | symmetric_ | |
| | CCC-ACC | |
| | 34_3-3_bulge- | |
| | symmetric_ | |
| | GAU-UGG | |
| | 53_3-3_bulge- | |
| | symmetric_ | |
| | AUG-GUG | |
| | 72_3-3_bulge- | |
| | symmetric_ | |
| | ACA-CCG | |
| 1554 | -1_4-4_bulge- | GAUAUUGUGACAUAUCUCUGGUAACA |
| | symmetric_ | UCACACAAAAGAUGAUUCUCUUCUAUA |
| | AUAU-CCAA | GGAUCCAAAUCGAGGGGGCAUUUUAA |
| | 16_4-4_bulge- | UCCAACUCUGAACUAAUCAUC |
| | symmetric_ | |
| | CCUG-AAUC | |
| | 36_4-4_bulge- | |
| | symmetric_ | |
| | UUUG-AUUC | |
| | 56_4-4_bulge- | |
| | symmetric_ | |
| | AGUG-GUAA | |
| 1555 | -4->0_5-5_ | GAUAUUGUGACAUAUCGACCAACUCAU |
| | internal_ | CACACAAAAGUCCGUAAUCUUCUAUAG |
| | loop-symmetric_ | GAUCGUCCUGGAGGGGGCAUUUUAAC |
| | AUAUA-CUGAA | UGAACUCUGAACUAAUCAUC |
| | 17_5-5_internal_ | |
| | loop-symmetric_ | |
| | CUGUG-GUCCU | |
| | 38_5-5_internal_ | |
| | loop-symmetric_ | |
| | UGCAU-UCCGU | |
| | 59_5-5_internal_ | |
| | loop-symmetric_ | |
| | GCAGA-GACCA | |
| 1556 | -4->1_6-6_ | GAUAUUGUGACAGUGAAGUGCACUCA |
| | internal_ | UCACACAAUUAAAUCAAAUCUUCUAUA |
| | loop-symmetric_ | GGAAAGAAUGGGAGGGGGCAUUUUAU |
| | AUAUAU- | AAGACCUCUGAACUAAUCAUC |
| | UAAGAC | |
| | 18_6-6_internal_ | |
| | loop-symmetric_ | |
| | UGUGGA-AAGAAU | |
| | 40_6-6_internal_ | |
| | loop-symmetric_ | |
| | CAUCUU-UUAAAU | |
| | 62_6-6_internal_ | |
| | loop-symmetric_ | |
| | GAGAUA-GUGAAG | |
| 1557 | -4->2_7-7_ | GAUAUUGUUUUUCACCUCUGCACUCAU |
| | internal_ | CACAGUUCAACUGCAAAUCUUCUAUAG |
| | loop-symmetric_ | CAGAAAAAGGGAGGGGGCAUUUUCUA |
| | AUAUAUU- | UUUACUCUGAACUAAUCAUC |
| | CUAUUUA | |
| | 19_7-7_internal_ | |
| | loop-symmetric_ | |
| | GUGGAUC- | |
| | CAGAAAA | |
| | 42_7-7_internal_ | |
| | loop-symmetric_ | |
| | UCUUUUG- | |
| | GUUCAAC | |
| | 65_7-7_internal_ | |
| | loop-symmetric_ | |
| | AUAUGUC- | |
| | UUUUCAC | |
| 1558 | -4->3_8-8_ | GAUACGAGAAGUUAUCUCUGCACUCAU |
| | internal_ | CCUUGAUCCGAUGCAAAUCUUCUAUCC |
| | loop-symmetric_ | GUUAGUCAGGGAGGGGGCAUUUGCCU |
| | AUAUAUUA- | AAAACUCUGAACUAAUCAUC |
| | GCCUAAAA | |
| | 20_8-8_internal_ | |
| | loop-symmetric_ | |
| | UGGAUCCU- | |
| | CCGUUAGU | |
| | 44_8-8_internal_ | |
| | loop-symmetric_ | |
| | UUUUGUGU- | |
| | CUUGAUCC | |
| | 68_8-8_internal_ | |
| | loop-symmetric_ | |
| | UGUCACAA- | |
| | CGAGAAGU | |
| 1559 | 0->1_2-2_bulge- | GAUAUUGUGACAGCUCUCUGCACUCAU |
| | symmetric_ | CACACAAUCGAUGCAAAUCUUCUAUAG |
| | AU-UG | GAUACACAGGGAGGGGGCAUUUUAUG |
| | 22_2-2_bulge- | AUAUCUCUGAACUAAUCAUC |
| | symmetric_ | |
| | GA-UA | |
| | 44_2-2_bulge- | |
| | symmetric_ | |
| | UU-UC | |
| | 66_2-2_bulge- | |
| | symmetric_ | |
| | UA-GC | |
| 1560 | 0->2_3-3_bulge- | GAUAUUGUCCGAUAUCUCUGCACUCAU |
| | symmetric_ | CACAAACAAGAUGCAAAUCUUCUAUAG |
| | AUU-UCG | UUCCCACAGGGAGGGGGCAUUUUUCGA |
| | 23_3-3_bulge- | UAUCUCUGAACUAAUCAUC |
| | symmetric_ | |
| | AUC-UUC | |
| | 46_3-3_bulge- | |
| | symmetric_ | |
| | UUG-AAC | |
| | 69_3-3_bulge- | |
| | symmetric_ | |
| | GUC-CCG | |
| 1561 | 0->3_4-4_bulge- | GAUAGGCCGACAUAUCUCUGCACUCAU |
| | symmetric_ | CUGUGAAAAGAUGCAAAUCUUCUAUC |
| | AUUA-GUAG | UACUCCACAGGGAGGGGGCAUUUGUA |
| | 24_4-4_bulge- | GAUAUCUCUGAACUAAUCAUC |
| | symmetric_ | |
| | UCCU-CUAC | |
| | 48_4-4_bulge- | |
| | symmetric_ | |
| | GUGU-UGUG | |
| | 72_4-4_bulge- | |
| | symmetric_ | |
| | ACAA-GGCC | |
| 1562 | 0->4_5-5_internal_ | GAUAUUGUGACAUAUCUCUGCACUCCC |
| | loop-symmetric_ | GUGACAAAAGAUGCAAAUCUUCUUUC |
| | AUUAA-CGUUC | AUAUCCACAGGGAGGGGGCAUUCGUUC |
| | 25_5-5_internal_ | AUAUCUCUGAACUAAUCAUC |
| | loop-symmetric_ | |
| | CCUAU-UUCAU | |
| | 50_5-5_internal_ | |
| | loop-symmetric_ | |
| | GUGAU-CCGUG | |
| 1563 | 0->5_6-6_internal_ | GAUAUUGUGACAUAUCUCUGCAGCGCU |
| | loop-symmetric_ | AACACAAAAGAUGCAAAUCUUGGAGCC |
| | AUUAAA- | GAUCCACAGGGAGGGGGCAUCGUCACA |
| | CGUCAC | UAUCUCUGAACUAAUCAUC |
| | 26_6-6_internal_ | |
| | loop-symmetric_ | |
| | CUAUAG-GGAGCC | |
| | 52_6-6_internal_ | |
| | loop-symmetric_ | |
| | GAUGAG-GCGCUA | |
| 1564 | 0->6_7-7_internal_ | GAUAUUGUGACAUAUCUCUAAAGAAU |
| | loop-symmetric_ | UCACACAAAAGAUGCAAAUCAUAUUU |
| | AUUAAAA- | UGGAUCCACAGGGAGGGGGCAGAUUC |
| | GAUUCUA | UAAUAUCUCUGAACUAAUCAUC |
| | 27_7-7_internal_ | |
| | loop-symmetric_ | |
| | UAUAGAA- | |
| | AUAUUUU | |
| | 54_7-7_internal_ | |
| | loop-symmetric_ | |
| | UGAGUGC- | |
| | AAAGAAU | |
| 1565 | 0->7_8-8_internal_ | GAUAUUGUGACAUAUCGAGAGUGACA |
| | loop-symmetric_ | UCACACAAAAGAUGCAAACGGUAUUA |
| | AUUAAAAU- | AGGAUCCACAGGGAGGGGGCCUAUAAC |
| | CUAUAACC | CAUAUCUCUGAACUAAUCAUC |
| | 28_8-8_internal_ | |
| | loop-symmetric_ | |
| | AUAGAAGA- | |
| | CGGUAUUA | |
| | 56_8-8_internal_ | |
| | loop-symmetric_ | |
| | AGUGCAGA- | |
| | GAGAGUGA | |
| 1566 | -6_6-6_internal_ | GCCAUUGUGUGAUAUCUGAGCACUCCG |
| | loop-symmetric_ | CACACACCAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 45_2-2_bulge- | |
| | symmetric_ | |
| | UU-CC | |
| | 53_2-2_bulge- | |
| | symmetric_ | |
| | AU-CG | |
| | 61_2-2_bulge- | |
| | symmetric_ | |
| | AG-GA | |
| | 69_2-2_bulge- | |
| | symmetric_ | |
| | GU-UG | |
| | 77_2-2_bulge- | |
| | symmetric_ | |
| | AU-CC | |
| 1567 | -6_6-6_internal_ | GAUAUGCAGACAUAGACCUGCACCUUU |
| | loop-symmetric_ | CACACUAUAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 45_3-3_bulge- | |
| | symmetric_ | |
| | UUU-UAU | |
| | 54_3-3_bulge- | |
| | symmetric_ | |
| | UGA-CUU | |
| | 63_3-3_bulge- | |
| | symmetric_ | |
| | AGA-GAC | |
| | 72_3-3_bulge- | |
| | symmetric_ | |
| | ACA-GCA | |
| 1568 | -6_6-6_internal_ | GUUCGUGUGACCAAACUCUGCCAAGAU |
| | loop-symmetric_ | CACAGUAUAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 45_4-4_bulge- | |
| | symmetric_ | |
| | UUUG-GUAU | |
| | 55_4-4_bulge- | |
| | symmetric_ | |
| | GAGU-CAAG | |
| | 65_4-4_bulge- | |
| | symmetric_ | |
| | AUAU-CAAA | |
| | 75_4-4_bulge- | |
| | symmetric_ | |
| | AUAU-UUCG | |
| 1569 | -6_6-6_internal_ | GAUAUUGUCCAAAAUCUCUCUUUCCAU |
| | loop-symmetric_ | CACUGUUCAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 45_5-5_internal_ | |
| | loop-symmetric_ | |
| | UUUGU-UGUUC | |
| | 56_5-5_internal_ | |
| | loop-symmetric_ | |
| | AGUGC-CUUUC | |
| | 67_5-5_internal_ | |
| | loop-symmetric_ | |
| | AUGUC-CCAAA | |
| 1570 | -6_6-6_internal_ | GUGAUUGUGACUAAUCUCUGCCGUCAU |
| | loop-symmetric_ | CACAAUAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 47_2-2_bulge- | |
| | symmetric_ | |
| | UG-AU | |
| | 57_2-2_bulge- | |
| | symmetric_ | |
| | GU-CG | |
| | 67_2-2_bulge- | |
| | symmetric_ | |
| | AU-UA | |
| | 77_2-2_bulge- | |
| | symmetric_ | |
| | AU-UG | |
| 1571 | -6_6-6_internal_ | GAUAUUGUCCAAUAUCUCUAAUCUCAU |
| | loop-symmetric_ | CACUGUAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 47_3-3_bulge- | |
| | symmetric_ | |
| | UGU-UGU | |
| | 58_3-3_bulge- | |
| | symmetric_ | |
| | UGC-AAU | |
| | 69_3-3_bulge- | |
| | symmetric_ | |
| | GUC-CCA | |
| 1572 | -6_6-6_internal_ | GAUAUCUUUACAUAUCUGGUGACUCA |
| | loop-symmetric_ | UCAACCUAAAGAUGCCUGACCUCUAUA |
| | UUCAGA-GGAACU | GGAUCCACAGGGAGGGGGCAUUUCAA |
| | 3_1-1_mismatch_ | UAUAUCGGAACUCUAAUCAUC |
| | A-C | |
| | 33_6-6_internal_ | |
| | loop-symmetric_ | |
| | AGAUUU-CUGACC | |
| | 47_4-4_bulge- | |
| | symmetric_ | |
| | UGUG-ACCU | |
| | 59_4-4_bulge- | |
| | symmetric_ | |
| | GCAG-GGUG | |
| | 71_4-4_bulge- | |
| | symmetric_ | |
| | CACA-CUUU | |
| 1573 | -6_6-6_internal_ | GAAAGUCUGACAUAUGCACCCACUCAU |
| | loop-symmetric_ | CUGAAUAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 47_5-5_internal_ | |
| | loop-symmetric_ | |
| | UGUGU-UGAAU | |
| | 60_5-5_internal_ | |
| | loop-symmetric_ | |
| | CAGAG-GCACC | |
| | 73_5-5_internal_ | |
| | loop-symmetric_ | |
| | CAAUA-AAGUC | |
| 1574 | -6_6-6_internal_ | GAUAUACUGACAUAUCUAAGCACUCAU |
| | loop-symmetric_ | CAGCCAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 49_2-2_bulge- | |
| | symmetric_ | |
| | UG-GC | |
| | 61_2-2_bulge- | |
| | symmetric_ | |
| | AG-AA | |
| | 73_2-2_bulge- | |
| | symmetric_ | |
| | CA-AC | |
| 1575 | -6_6-6_internal_ | GAGUGUGUGACAUAUGAGUGCACUCA |
| | loop-symmetric_ | UCCUUCAAAAGAUGCCUGACCUCUAUA |
| | UUCAGA-GGAACU | GGAUCCACAGGGAGGGGGCAUUUCAA |
| | 3_1-1_mismatch_ | UAUAUCGGAACUCUAAUCAUC |
| | A-C | |
| | 33_6-6_internal_ | |
| | loop-symmetric_ | |
| | AGAUUU-CUGACC | |
| | 49_3-3_bulge- | |
| | symmetric_ | |
| | UGU-CUU | |
| | 62_3-3_bulge- | |
| | symmetric_ | |
| | GAG-GAG | |
| | 75_3-3_bulge- | |
| | symmetric_ | |
| | AUA-GUG | |
| 1576 | -6_6-6_internal_ | GAUAUUGUGACAUUGGCCUGCACUCAU |
| | loop-symmetric_ | GUAUCAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 49_4-4_bulge- | |
| | symmetric_ | |
| | UGUG-GUAU | |
| | 63_4-4_bulge- | |
| | symmetric_ | |
| | AGAU-UGGC | |
| 1577 | -6_6-6_internal_ | GAUAUUGUGACCUUAAUCUGCACUCAG |
| | loop-symmetric_ | CCUUCAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 49_5-5_internal_ | |
| | loop-symmetric_ | |
| | UGUGA-GCCUU | |
| | 64_5-5_internal_ | |
| | loop-symmetric_ | |
| | GAUAU-CUUAA | |
| 1578 | -6_6-6_internal_ | GAUAUUGUGACAUUGCUCUGCACUCAU |
| | loop-symmetric_ | GCCACAAAAGAUGCCUGACCUCUAUAG |
| | UUCAGA-GGAACU | |
| | 3_1-1_mismatch_ | |
| | A-C | |
| | 33_6-6_internal_ | |
| | loop-symmetric_ | |
| | AGAUUU-CUGACC | |
| | 51_2-2_bulge- | GAUCCACAGGGAGGGGGCAUUUCAAU |
| | symmetric_ | AUAUCGGAACUCUAAUCAUC |
| | UG-GC | |
| | 65_2-2_bulge- | |
| | symmetric_ | |
| | AU-UG | |
| 1579 | -6_6-6_internal_ | GAUAUUGUGACUACUCUCUGCACUCAA |
| | loop-symmetric_ | GCCACAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 51_3-3_bulge- | |
| | symmetric_ | |
| | UGA-AGC | |
| | 66_3-3_bulge- | |
| | symmetric_ | |
| | UAU-UAC | |
| 1580 | -6_6-6_internal_ | GAUAUUGUGUGUCAUCUCUGCACUCCU |
| | loop-symmetric_ | ACCACAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 51_4-4_bulge- | |
| | symmetric_ | |
| | UGAU-CUAC | |
| | 67_4-4_bulge- | |
| | symmetric_ | |
| | AUGU-UGUC | |
| 1581 | -6_6-6_internal_ | GAUAUUGGCAGUUAUCUCUGCACUGU |
| | loop-symmetric_ | AGUCACAAAAGAUGCCUGACCUCUAUA |
| | UUCAGA-GGAACU | GGAUCCACAGGGAGGGGGCAUUUCAA |
| | 3_1-1_mismatch_ | UAUAUCGGAACUCUAAUCAUC |
| | A-C | |
| | 33_6-6_internal_ | |
| | loop-symmetric_ | |
| | AGAUUU-CUGACC | |
| | 51_5-5_internal_ | |
| | loop-symmetric_ | |
| | UGAUG-GUAGU | |
| | 68_5-5_internal_ | |
| | loop-symmetric_ | |
| | UGUCA-GCAGU | |
| 1582 | -6_6-6_internal_ | GAUAUUGUGUAAUAUCUCUGCACUCU |
| | loop-symmetric_ | ACACACAAAAGAUGCCUGACCUCUAUA |
| | UUCAGA-GGAACU | GGAUCCACAGGGAGGGGGCAUUUCAA |
| | 3_1-1_mismatch_ | UAUAUCGGAACUCUAAUCAUC |
| | A-C | |
| | 33_6-6_internal_ | |
| | loop-symmetric_ | |
| | AGAUUU-CUGACC | |
| | 53_2-2_bulge- | |
| | symmetric_ | |
| | AU-UA | |
| | 69_2-2_bulge- | |
| | symmetric_ | |
| | GU-UA | |
| 1583 | -6_6-6_internal_ | GAUAUUGAUUCAUAUCUCUGCACUGCG |
| | loop-symmetric_ | CACACAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 53_3-3_bulge- | |
| | symmetric_ | |
| | AUG-GCG | |
| | 70_3-3_bulge- | |
| | symmetric_ | |
| | UCA-AUU | |
| 1584 | -6_6-6_internal_ | GAUAUAUAAACAUAUCUCUGCACAGCC |
| | loop-symmetric_ | CACACAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 53_4-4_bulge- | |
| | symmetric_ | |
| | AUGA-AGCC | |
| | 71_4-4_bulge- | |
| | symmetric_ | |
| | CACA-AUAA | |
| 1585 | -6_6-6_internal_ | GAUUCAAGGACAUAUCUCUGCAGAAA |
| | loop-symmetric_ | ACACACAAAAGAUGCCUGACCUCUAUA |
| | UUCAGA-GGAACU | GGAUCCACAGGGAGGGGGCAUUUCAA |
| | 3_1-1_mismatch_ | UAUAUCGGAACUCUAAUCAUC |
| | A-C | |
| | 33_6-6_internal_ | |
| | loop-symmetric_ | |
| | AGAUUU-CUGACC | |
| | 53_5-5_internal_ | |
| | loop-symmetric_ | |
| | AUGAG-GAAAA | |
| | 72_5-5_internal_ | |
| | loop-symmetric_ | |
| | ACAAU-UCAAG | |
| 1586 | -6_6-6_internal_ | GAUAUCCUGACAUAUCUCUGCACAAAU |
| | loop-symmetric_ | CACACAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 55_2-2_bulge- | |
| | symmetric_ | |
| | GA-AA | |
| | 73_2-2_bulge- | |
| | symmetric_ | |
| | CA-CC | |
| 1587 | -6_6-6_internal_ | GAUUCAGUGACAUAUCUCUGCAGCGAU |
| | loop-symmetric_ | CACACAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 55_3-3_bulge- | |
| | symmetric_ | |
| | GAG-GCG | |
| | 74_3-3_bulge- | |
| | symmetric_ | |
| | AAU-UCA | |
| 1588 | -6_6-6_internal_ | GCGCGUGUGACAUAUCUCUGCCAAAAU |
| | loop-symmetric_ | CACACAAAAGAUGCCUGACCUCUAUAG |
| | UUCAGA-GGAACU | GAUCCACAGGGAGGGGGCAUUUCAAU |
| | 3_1-1_mismatch_ | AUAUCGGAACUCUAAUCAUC |
| | A-C | |
| | 33_6-6_internal_ | |
| | loop-symmetric_ | |
| | AGAUUU-CUGACC | |
| | 55_4-4_bulge- | |
| | symmetric_ | |
| | GAGU-CAAA | |
| | 75_4-4_bulge- | |
| | symmetric_ | |
| | AUAU-CGCG | |
| 1589 | -6_6-6_internal_ | GAUAUUGUGACAUAUCUCUGAUGGGA |
| | loop-symmetric_ | UCACACAAAAGAUGCCUGACCUCUAUA |
| | UUCAGA-GGAACU | GGAUCCACAGGGAGGGGGCAUUUCAA |
| | 3_1-1_mismatch_ | UAUAUCGGAACUCUAAUCAUC |
| | A-C | |
| | 33_6-6_internal_ | |
| | loop-symmetric_ | |
| | AGAUUU-CUGACC | |
| | 55_5-5_internal_ | |
| | loop-symmetric_ | |
| | GAGUG-AUGGG | |

TABLE 2
Percent Editing of As in ATTAAA of DUX4 mRNA by ADAR1, ADAR2, or ADAR1 and ADAR2

| SEQ | | | | | | | | | | | | | | | |
|---|---|---|---|---|---|---|---|---|---|---|---|---|---|---|---|
| ID | A1/ | A1/ | A1/ | A1/ | A1/ | A2/ | A2/ | A2/ | A2/ | A2/ | A1 + | A1 + | A1 + | A1 + | A1 + |
| NO | P0 | P3 | P4 | P5 | Any | P0 | P3 | P4 | P5 | Any | 2/P0 | 2/P3 | 2/P4 | 2/P5 | 2/Any |
| 2 | 30.6% | 58.0% | 30.4% | 22.9% | 70.7% | 28.5% | 59.9% | 40.6% | 41.6% | 87.0% | 29.0% | 60.7% | 39.1% | 33.8% | 87.3% |
| 3 | 20.4% | 64.7% | 35.9% | 30.8% | 74.4% | 11.4% | 69.8% | 52.2% | 46.3% | 89.1% | 15.0% | 70.7% | 45.0% | 38.1% | 89.6% |
| 4 | 14.5% | 57.7% | 37.7% | 21.6% | 72.1% | 9.4% | 61.7% | 48.9% | 46.8% | 89.3% | 12.6% | 62.4% | 45.9% | 37.0% | 89.2% |
| 5 | 14.3% | 60.1% | 32.5% | 31.8% | 73.8% | 7.3% | 59.6% | 49.6% | 46.7% | 89.4% | 12.2% | 63.6% | 46.8% | 42.9% | 89.9% |
| 6 | 15.3% | 61.4% | 30.7% | 23.0% | 75.5% | 9.9% | 63.3% | 44.8% | 37.7% | 87.8% | 12.5% | 64.3% | 41.2% | 34.2% | 88.9% |
| 7 | 43.1% | 68.0% | 25.5% | 10.6% | 78.4% | 30.3% | 49.1% | 43.5% | 45.1% | 88.3% | 35.1% | 53.9% | 42.7% | 39.8% | 90.2% |
| 8 | 17.6% | 81.0% | 23.3% | 8.9% | 86.4% | 11.8% | 80.6% | 69.4% | 55.6% | 93.5% | 13.6% | 82.5% | 59.4% | 43.9% | 94.1% |
| 9 | 17.4% | 65.7% | 38.0% | 9.8% | 79.9% | 7.8% | 56.9% | 49.7% | 50.1% | 90.1% | 13.4% | 61.3% | 50.3% | 44.3% | 91.2% |
| 10 | 10.6% | 76.9% | 23.6% | 11.0% | 84.1% | 8.5% | 49.4% | 56.0% | 52.5% | 90.8% | 9.6% | 52.3% | 47.3% | 47.7% | 91.5% |
| 11 | 16.7% | 67.5% | 24.1% | 10.2% | 79.9% | 7.1% | 63.2% | 53.4% | 51.8% | 91.7% | 10.6% | 62.7% | 50.1% | 47.0% | 91.6% |
| 12 | 37.0% | 65.2% | 26.7% | 12.6% | 77.7% | 31.9% | 52.9% | 45.1% | 39.2% | 88.9% | 35.9% | 56.2% | 41.4% | 35.6% | 89.3% |
| 13 | 18.3% | 71.6% | 30.1% | 16.8% | 78.7% | 13.5% | 68.3% | 54.7% | 37.3% | 88.0% | 14.4% | 74.0% | 49.6% | 27.8% | 90.4% |
| 14 | 10.1% | 77.5% | 19.5% | 14.4% | 84.8% | 8.2% | 52.9% | 57.9% | 55.0% | 92.0% | 9.0% | 53.9% | 45.6% | 45.9% | 91.9% |
| 15 | 22.9% | 77.3% | 22.9% | 8.9% | 88.0% | 7.8% | 70.7% | 62.6% | 54.1% | 93.3% | 10.3% | 70.8% | 59.2% | 49.4% | 94.8% |
| 16 | 35.8% | 69.8% | 39.8% | 23.2% | 81.7% | 36.5% | 61.7% | 40.9% | 35.9% | 88.8% | 34.3% | 58.6% | 36.4% | 30.2% | 91.3% |
| 17 | 22.6% | 82.5% | 43.1% | 41.2% | 87.9% | 19.1% | 82.0% | 67.9% | 44.9% | 93.5% | 13.7% | 81.8% | 50.3% | 28.2% | 93.1% |
| 18 | 16.1% | 66.1% | 45.7% | 20.1% | 77.5% | 10.0% | 56.7% | 48.9% | 42.6% | 85.8% | 11.8% | 58.0% | 44.1% | 31.9% | 87.8% |
| 19 | 12.1% | 72.3% | 40.5% | 44.0% | 82.3% | 10.1% | 55.2% | 50.0% | 47.9% | 89.9% | 10.0% | 54.8% | 42.6% | 41.9% | 90.8% |
| 20 | 16.1% | 67.8% | 31.8% | 22.2% | 79.4% | 8.7% | 63.0% | 48.0% | 40.1% | 89.7% | 13.1% | 63.6% | 43.0% | 34.6% | 89.4% |
| 21 | 42.3% | 69.2% | 28.6% | 26.0% | 86.3% | 50.6% | 61.5% | 47.7% | 42.3% | 91.4% | 46.8% | 58.3% | 39.7% | 30.4% | 92.6% |
| 22 | 16.9% | 70.9% | 41.7% | 42.1% | 79.4% | 24.7% | 74.0% | 51.3% | 36.5% | 90.1% | 18.2% | 72.8% | 42.8% | 27.6% | 90.5% |
| 23 | 16.2% | 54.2% | 33.8% | 16.0% | 83.2% | 15.3% | 59.9% | 49.6% | 41.6% | 90.3% | 15.2% | 52.7% | 40.7% | 24.5% | 90.9% |
| 24 | 11.2% | 66.1% | 40.6% | 48.7% | 82.8% | 10.7% | 57.2% | 47.1% | 46.5% | 90.4% | 10.4% | 54.9% | 38.9% | 39.8% | 91.2% |
| 25 | 11.6% | 65.4% | 51.7% | 37.3% | 78.6% | 11.7% | 61.1% | 41.8% | 37.3% | 87.9% | 11.9% | 54.7% | 36.4% | 28.4% | 86.3% |
| 26 | 36.0% | 53.0% | 22.1% | 17.6% | 74.5% | 38.4% | 60.7% | 35.9% | 33.3% | 85.5% | 36.3% | 59.7% | 34.7% | 27.4% | 87.8% |
| 27 | 21.8% | 71.2% | 24.1% | 26.3% | 80.7% | 32.1% | 70.2% | 48.5% | 33.3% | 87.1% | 23.0% | 75.5% | 44.7% | 26.7% | 90.6% |
| 28 | 14.2% | 46.9% | 26.4% | 15.3% | 77.5% | 22.6% | 64.5% | 50.2% | 39.7% | 90.7% | 14.5% | 54.2% | 36.1% | 23.4% | 89.5% |
| 29 | 12.3% | 58.6% | 24.3% | 36.6% | 81.7% | 17.3% | 61.4% | 42.9% | 43.2% | 89.6% | 12.3% | 60.7% | 38.0% | 37.8% | 91.7% |
| 30 | 11.9% | 66.3% | 30.0% | 33.5% | 86.9% | 24.8% | 68.9% | 44.9% | 40.1% | 92.1% | 13.9% | 69.0% | 44.2% | 33.6% | 93.0% |
| 31 | 36.4% | 57.9% | 24.1% | 18.4% | 73.3% | 37.2% | 60.9% | 35.3% | 29.1% | 86.6% | 40.8% | 62.3% | 34.3% | 25.8% | 88.7% |
| 32 | 25.6% | 67.8% | 21.7% | 24.8% | 80.1% | 31.6% | 73.8% | 52.4% | 37.2% | 89.7% | 20.7% | 77.1% | 47.9% | 26.5% | 91.7% |
| 33 | 15.2% | 42.3% | 24.8% | 11.9% | 80.9% | 25.2% | 62.3% | 48.0% | 41.2% | 90.4% | 15.1% | 54.6% | 39.1% | 23.5% | 89.9% |
| 34 | 14.5% | 56.0% | 22.6% | 28.2% | 75.5% | 12.3% | 62.2% | 42.1% | 39.1% | 88.7% | 12.5% | 60.6% | 35.8% | 34.7% | 89.6% |
| 35 | 18.3% | 52.0% | 22.4% | 18.3% | 74.0% | 16.1% | 63.8% | 38.2% | 31.7% | 87.5% | 16.1% | 61.5% | 35.6% | 25.8% | 87.2% |
| 36 | 37.5% | 58.7% | 22.7% | 15.7% | 74.1% | 37.8% | 63.8% | 43.0% | 30.3% | 86.7% | 35.9% | 60.7% | 36.7% | 24.6% | 86.7% |
| 37 | 24.3% | 57.4% | 22.0% | 16.7% | 71.0% | 23.5% | 66.6% | 50.3% | 37.7% | 87.9% | 20.1% | 66.8% | 39.3% | 21.9% | 86.7% |
| 38 | 20.3% | 47.8% | 28.6% | 11.7% | 78.9% | 25.1% | 62.5% | 49.0% | 31.1% | 88.5% | 21.4% | 60.6% | 43.7% | 23.9% | 90.5% |
| 39 | 17.2% | 49.9% | 18.9% | 17.6% | 72.2% | 11.2% | 63.2% | 44.5% | 39.7% | 88.0% | 14.4% | 56.8% | 31.3% | 27.6% | 87.9% |
| 40 | 18.1% | 51.7% | 21.2% | 15.7% | 72.2% | 13.9% | 63.7% | 41.3% | 34.0% | 84.5% | 14.3% | 61.2% | 36.6% | 26.3% | 87.3% |
| 41 | 29.0% | 54.4% | 24.0% | 18.0% | 67.9% | 25.0% | 53.3% | 34.4% | 27.7% | 84.1% | 26.7% | 56.3% | 34.2% | 24.1% | 85.1% |
| 42 | 25.0% | 59.4% | 30.0% | 21.4% | 71.5% | 13.9% | 62.3% | 47.1% | 40.9% | 86.6% | 19.0% | 68.2% | 44.1% | 27.4% | 87.9% |
| 43 | 14.1% | 64.9% | 39.4% | 19.4% | 80.5% | 15.6% | 62.0% | 44.2% | 36.2% | 87.0% | 17.9% | 68.7% | 48.6% | 30.2% | 90.4% |
| 44 | 17.1% | 56.6% | 22.3% | 18.5% | 72.6% | 10.3% | 60.3% | 33.9% | 32.4% | 84.8% | 14.5% | 60.0% | 32.8% | 27.9% | 86.4% |
| 45 | 16.5% | 61.0% | 29.3% | 20.4% | 73.1% | 7.2% | 57.2% | 39.2% | 41.2% | 85.0% | 11.1% | 64.9% | 38.2% | 36.8% | 87.8% |
| 46 | 32.1% | 55.7% | 25.1% | 14.3% | 67.9% | 23.8% | 56.2% | 36.8% | 30.1% | 82.8% | 29.4% | 58.7% | 33.2% | 21.5% | 84.9% |
| 47 | 22.3% | 59.9% | 30.2% | 20.5% | 71.1% | 12.6% | 61.3% | 45.6% | 37.3% | 85.0% | 17.6% | 67.0% | 43.5% | 28.0% | 86.4% |
| 48 | 15.7% | 56.9% | 35.0% | 20.8% | 72.0% | 8.2% | 55.4% | 40.3% | 38.2% | 86.1% | 13.6% | 62.0% | 43.2% | 34.9% | 87.5% |
| 49 | 15.4% | 55.2% | 25.0% | 19.3% | 70.0% | 8.4% | 59.5% | 36.8% | 31.5% | 85.9% | 13.7% | 60.6% | 34.3% | 26.5% | 86.0% |
| 50 | 21.8% | 63.3% | 25.5% | 16.9% | 78.6% | 10.4% | 67.0% | 49.0% | 33.1% | 89.7% | 15.1% | 67.4% | 41.1% | 27.0% | 90.6% |
| 51 | 29.5% | 57.3% | 29.6% | 15.7% | 67.7% | 20.0% | 56.4% | 39.6% | 33.6% | 82.8% | 28.7% | 57.4% | 36.5% | 25.9% | 83.9% |
| 52 | 27.8% | 67.5% | 41.0% | 25.2% | 76.4% | 9.7% | 64.6% | 48.9% | 45.5% | 84.3% | 17.6% | 69.6% | 50.2% | 36.2% | 87.7% |
| 53 | 18.5% | 55.6% | 35.9% | 15.7% | 71.3% | 9.9% | 58.9% | 45.7% | 41.0% | 85.7% | 15.7% | 61.0% | 42.0% | 27.1% | 86.1% |
| 54 | 14.1% | 59.8% | 28.7% | 21.3% | 74.1% | 8.3% | 56.5% | 37.8% | 33.1% | 85.5% | 13.7% | 63.5% | 36.3% | 28.8% | 88.0% |
| 55 | 17.1% | 60.3% | 33.0% | 16.1% | 72.3% | 7.9% | 61.0% | 43.1% | 36.9% | 86.0% | 13.4% | 63.3% | 38.3% | 27.6% | 86.9% |
| 56 | 34.2% | 62.1% | 37.9% | 18.0% | 73.5% | 17.2% | 58.9% | 39.3% | 39.3% | 86.0% | 28.6% | 62.1% | 41.7% | 31.4% | 87.2% |
| 57 | 21.5% | 63.5% | 42.7% | 28.5% | 74.0% | 8.8% | 62.4% | 45.6% | 43.3% | 86.6% | 16.9% | 68.1% | 46.6% | 33.9% | 88.5% |
| 58 | 16.9% | 57.2% | 37.1% | 15.6% | 72.9% | 9.2% | 59.5% | 43.6% | 41.8% | 88.0% | 14.4% | 60.4% | 42.0% | 30.4% | 87.9% |
| 59 | 14.0% | 56.8% | 30.6% | 22.9% | 70.2% | 8.4% | 56.9% | 42.7% | 36.8% | 86.2% | 14.0% | 60.8% | 40.8% | 33.0% | 87.6% |
| 60 | 16.7% | 59.9% | 33.7% | 18.0% | 73.4% | 7.6% | 57.2% | 40.5% | 37.1% | 84.7% | 14.2% | 60.9% | 37.3% | 28.3% | 87.2% |
| 61 | 29.5% | 61.2% | 39.5% | 25.0% | 71.9% | 19.0% | 54.5% | 36.7% | 40.5% | 85.5% | 25.8% | 62.6% | 43.1% | 37.9% | 88.0% |
| 62 | 20.2% | 59.9% | 40.6% | 31.8% | 70.1% | 8.9% | 61.3% | 41.8% | 39.4% | 84.6% | 15.8% | 65.0% | 44.8% | 34.1% | 86.6% |
| 63 | 17.0% | 65.3% | 48.2% | 22.9% | 76.0% | 7.7% | 52.8% | 40.4% | 44.5% | 85.3% | 14.8% | 64.8% | 45.1% | 34.4% | 87.1% |
| 64 | 12.8% | 62.8% | 37.7% | 29.9% | 74.0% | 6.9% | 52.1% | 46.7% | 44.6% | 87.4% | 13.0% | 63.9% | 43.8% | 37.1% | 88.8% |
| 65 | 14.5% | 61.1% | 40.7% | 20.7% | 73.2% | 6.9% | 56.6% | 38.2% | 38.5% | 87.2% | 14.1% | 62.6% | 41.5% | 33.0% | 87.2% |
| 66 | 27.7% | 59.8% | 39.2% | 25.0% | 71.2% | 16.9% | 46.7% | 32.9% | 35.7% | 85.2% | 25.1% | 61.1% | 39.6% | 34.7% | 86.3% |
| 67 | 20.6% | 65.5% | 49.1% | 41.1% | 75.2% | 8.0% | 61.0% | 41.4% | 40.7% | 85.2% | 14.2% | 69.6% | 47.5% | 38.9% | 87.2% |
| 68 | 17.3% | 63.8% | 48.1% | 34.6% | 75.5% | 8.7% | 53.5% | 38.9% | 43.9% | 87.4% | 15.7% | 63.2% | 44.7% | 38.8% | 87.8% |
| 69 | 15.1% | 66.3% | 47.7% | 42.2% | 77.1% | 6.4% | 49.0% | 39.5% | 40.2% | 86.6% | 14.0% | 62.9% | 45.4% | 40.9% | 87.5% |
| 70 | 14.5% | 60.2% | 35.3% | 28.6% | 72.7% | 7.6% | 54.8% | 35.2% | 38.3% | 86.7% | 12.8% | 63.2% | 39.2% | 34.2% | 88.5% |
| 71 | 31.5% | 63.2% | 44.8% | 35.2% | 73.5% | 19.2% | 47.0% | 33.0% | 38.7% | 83.6% | 26.5% | 62.6% | 41.9% | 38.6% | 86.6% |
| 72 | 24.9% | 75.4% | 63.1% | 60.0% | 82.2% | 8.2% | 66.6% | 40.9% | 44.1% | 87.5% | 15.7% | 73.4% | 53.8% | 52.3% | 89.9% |
| 73 | 17.6% | 61.4% | 45.3% | 35.5% | 73.6% | 7.9% | 48.6% | 35.3% | 39.2% | 87.1% | 16.5% | 63.4% | 42.4% | 33.8% | 88.3% |
| 74 | 15.1% | 62.2% | 44.5% | 40.1% | 72.8% | 6.5% | 47.5% | 41.1% | 41.1% | 86.4% | 13.0% | 61.9% | 41.2% | 37.2% | 86.7% |
| 75 | 14.6% | 67.6% | 49.8% | 36.5% | 77.9% | 6.0% | 51.5% | 33.7% | 42.2% | 85.7% | 13.2% | 67.3% | 45.1% | 39.4% | 90.0% |
| 76 | 19.5% | 62.4% | 44.9% | 41.2% | 72.2% | 9.8% | 58.5% | 35.3% | 32.5% | 83.2% | 14.5% | 66.8% | 42.0% | 38.9% | 86.2% |
| 77 | 16.7% | 67.3% | 52.3% | 42.0% | 77.0% | 6.5% | 43.4% | 33.5% | 34.5% | 87.3% | 12.8% | 62.6% | 43.0% | 38.5% | 87.2% |
| 78 | 14.6% | 60.8% | 42.5% | 38.4% | 71.7% | 6.2% | 44.8% | 35.3% | 34.7% | 83.4% | 12.0% | 60.1% | 40.6% | 38.1% | 85.7% |
| 79 | 15.8% | 61.5% | 37.4% | 30.0% | 72.7% | 8.4% | 53.3% | 36.0% | 38.8% | 87.1% | 14.6% | 62.5% | 38.0% | 36.1% | 87.0% |
| 80 | 25.6% | 59.0% | 37.7% | 29.9% | 70.6% | 17.2% | 48.9% | 33.5% | 36.3% | 85.8% | 24.4% | 61.8% | 38.7% | 33.1% | 86.7% |
| 81 | 16.6% | 65.5% | 45.5% | 43.6% | 75.4% | 8.8% | 63.9% | 40.8% | 36.9% | 88.0% | 14.6% | 67.4% | 44.9% | 40.7% | 87.6% |
| 82 | 14.6% | 63.0% | 43.2% | 28.8% | 74.5% | 8.3% | 48.7% | 37.2% | 43.0% | 87.5% | 13.4% | 64.7% | 44.2% | 41.1% | 88.9% |
| 83 | 13.6% | 60.7% | 37.9% | 34.9% | 72.8% | 7.0% | 44.6% | 37.6% | 35.6% | 86.6% | 13.9% | 59.9% | 39.3% | 36.2% | 87.6% |
| 84 | 14.6% | 67.1% | 39.6% | 34.0% | 77.3% | 6.7% | 53.9% | 36.4% | 39.4% | 88.4% | 13.7% | 63.7% | 38.4% | 37.0% | 88.1% |
| 85 | 25.6% | 58.9% | 36.4% | 25.2% | 70.7% | 20.2% | 47.3% | 33.7% | 37.1% | 83.9% | 27.0% | 60.6% | 39.8% | 31.6% | 86.4% |
| 86 | 16.7% | 66.1% | 45.0% | 44.1% | 74.6% | 8.4% | 65.1% | 41.9% | 39.3% | 86.9% | 15.3% | 70.6% | 43.7% | 38.4% | 88.3% |
| 87 | 14.5% | 61.8% | 43.7% | 28.7% | 74.0% | 7.2% | 46.6% | 37.3% | 37.2% | 86.0% | 13.7% | 63.4% | 44.1% | 36.4% | 87.1% |
| 88 | 12.2% | 65.5% | 37.4% | 37.9% | 76.3% | 6.9% | 55.2% | 51.9% | 50.1% | 90.3% | 10.7% | 65.4% | 49.7% | 46.7% | 89.5% |
| 89 | 13.4% | 62.7% | 34.2% | 27.1% | 73.6% | 7.1% | 55.0% | 36.3% | 38.4% | 87.9% | 15.1% | 64.9% | 41.1% | 35.6% | 89.9% |
| 90 | 26.5% | 69.3% | 42.5% | 31.1% | 78.8% | 26.3% | 51.1% | 38.4% | 41.4% | 89.2% | 34.7% | 65.8% | 41.0% | 36.6% | 88.8% |
| 91 | 16.1% | 62.8% | 42.0% | 38.6% | 73.9% | 8.9% | 64.7% | 40.8% | 35.1% | 88.4% | 18.3% | 68.5% | 41.2% | 35.5% | 89.2% |
| 92 | 12.8% | 57.0% | 37.8% | 24.3% | 70.1% | 6.6% | 48.1% | 36.7% | 36.0% | 85.5% | 13.9% | 61.5% | 41.3% | 33.4% | 88.0% |
| 93 | 13.6% | 60.6% | 35.0% | 31.5% | 72.2% | 6.2% | 49.2% | 44.2% | 44.1% | 86.5% | 12.7% | 61.8% | 42.1% | 38.7% | 88.5% |
| 94 | 15.6% | 61.3% | 33.7% | 25.9% | 72.7% | 6.8% | 50.9% | 32.5% | 34.5% | 84.2% | 13.1% | 62.0% | 36.8% | 33.2% | 86.7% |
| 95 | 25.4% | 57.6% | 34.1% | 23.9% | 68.4% | 22.5% | 46.4% | 34.1% | 36.5% | 82.8% | 26.5% | 57.6% | 36.7% | 31.1% | 84.7% |
| 96 | 16.9% | 63.5% | 40.6% | 35.5% | 73.1% | 9.3% | 68.9% | 48.7% | 42.9% | 89.6% | 17.5% | 72.8% | 46.8% | 38.9% | 90.3% |
| 97 | 13.3% | 56.3% | 36.4% | 22.3% | 70.6% | 6.3% | 48.7% | 37.5% | 35.0% | 86.7% | 13.3% | 60.9% | 41.2% | 30.4% | 87.2% |
| 98 | 14.0% | 60.2% | 32.2% | 24.7% | 72.9% | 7.0% | 56.0% | 40.4% | 37.0% | 87.4% | 12.5% | 62.9% | 38.4% | 31.8% | 87.5% |
| 99 | 27.1% | 57.6% | 32.0% | 21.5% | 70.7% | 23.6% | 50.6% | 37.4% | 36.9% | 86.2% | 29.5% | 58.6% | 40.3% | 33.7% | 87.2% |
| 100 | 19.2% | 62.2% | 38.6% | 33.6% | 70.9% | 10.0% | 70.6% | 49.3% | 42.7% | 89.1% | 16.4% | 72.6% | 47.3% | 36.2% | 89.1% |
| 101 | 15.3% | 58.5% | 38.7% | 21.1% | 71.5% | 8.0% | 53.3% | 43.3% | 41.1% | 88.0% | 13.3% | 61.1% | 44.3% | 34.6% | 89.2% |
| 102 | 14.2% | 56.2% | 31.2% | 27.2% | 68.5% | 7.7% | 48.5% | 42.9% | 42.7% | 86.7% | 12.8% | 56.9% | 42.9% | 40.4% | 87.5% |
| 103 | 15.1% | 58.8% | 32.1% | 24.1% | 72.6% | 7.7% | 56.5% | 37.1% | 39.2% | 86.5% | 11.9% | 60.5% | 37.9% | 34.0% | 90.0% |
| 104 | 29.1% | 57.9% | 32.0% | 21.0% | 70.8% | 30.4% | 55.9% | 40.9% | 41.7% | 87.1% | 32.2% | 58.6% | 38.2% | 36.1% | 87.9% |
| 105 | 20.0% | 62.1% | 38.7% | 31.6% | 73.2% | 9.1% | 67.7% | 47.4% | 41.8% | 87.7% | 15.4% | 71.1% | 45.4% | 37.4% | 89.1% |
| 106 | 15.8% | 58.1% | 37.9% | 22.5% | 71.3% | 7.8% | 51.8% | 40.9% | 42.3% | 88.7% | 12.4% | 60.8% | 45.2% | 38.1% | 88.7% |
| 107 | 14.7% | 61.3% | 33.1% | 30.1% | 73.2% | 6.8% | 55.1% | 47.3% | 46.3% | 88.3% | 12.3% | 63.0% | 44.6% | 42.9% | 89.6% |
| 108 | 17.0% | 59.8% | 31.2% | 21.7% | 72.1% | 7.1% | 60.1% | 45.4% | 47.2% | 90.1% | 13.4% | 63.1% | 42.0% | 41.2% | 91.6% |
| 109 | 32.0% | 58.6% | 33.5% | 20.8% | 69.1% | 30.2% | 56.8% | 42.2% | 42.6% | 87.6% | 34.2% | 59.8% | 40.0% | 37.5% | 87.4% |
| 110 | 24.0% | 61.9% | 39.4% | 32.8% | 71.9% | 11.3% | 69.6% | 53.2% | 46.2% | 89.1% | 16.9% | 73.3% | 50.2% | 40.7% | 89.9% |
| 111 | 16.8% | 56.3% | 37.2% | 20.0% | 70.0% | 8.4% | 58.0% | 46.2% | 45.7% | 88.7% | 13.5% | 58.2% | 44.6% | 40.2% | 89.0% |
| 112 | 14.6% | 57.8% | 31.0% | 27.4% | 71.0% | 5.6% | 53.6% | 49.5% | 47.0% | 88.4% | 12.2% | 63.8% | 49.7% | 47.1% | 91.1% |
| 113 | 17.3% | 65.9% | 34.2% | 21.9% | 77.0% | 7.6% | 64.5% | 46.9% | 45.7% | 89.4% | 12.3% | 67.3% | 42.3% | 40.0% | 92.0% |
| 114 | 36.1% | 61.4% | 34.6% | 22.4% | 72.1% | 36.3% | 62.3% | 49.1% | 47.6% | 89.7% | 39.0% | 64.1% | 45.4% | 41.0% | 90.4% |
| 115 | 31.1% | 70.0% | 47.6% | 38.0% | 78.4% | 13.0% | 75.2% | 60.1% | 50.0% | 91.0% | 18.8% | 78.8% | 57.1% | 45.2% | 92.1% |
| 116 | 19.7% | 59.4% | 41.2% | 23.3% | 73.6% | 8.9% | 64.7% | 55.6% | 55.0% | 91.7% | 14.6% | 72.3% | 57.5% | 47.7% | 92.6% |
| 117 | 21.9% | 60.9% | 33.9% | 21.0% | 73.9% | 6.6% | 65.7% | 51.9% | 48.9% | 90.9% | 13.7% | 67.7% | 42.7% | 38.1% | 91.3% |
| 118 | 36.9% | 62.2% | 32.6% | 23.6% | 72.8% | 30.6% | 57.8% | 43.7% | 42.8% | 88.9% | 36.0% | 64.7% | 41.6% | 35.0% | 87.0% |
| 119 | 20.1% | 61.0% | 40.2% | 22.0% | 72.1% | 9.5% | 60.1% | 52.4% | 50.9% | 89.9% | 13.4% | 69.3% | 53.8% | 42.2% | 89.9% |
| 120 | 17.2% | 57.7% | 31.1% | 27.5% | 69.7% | 8.1% | 54.7% | 49.1% | 46.3% | 88.9% | 16.2% | 62.6% | 46.8% | 43.8% | 88.0% |
| 121 | 23.7% | 64.2% | 33.5% | 22.3% | 74.9% | 8.8% | 62.9% | 48.6% | 44.7% | 89.9% | 14.0% | 69.7% | 45.2% | 38.2% | 90.2% |
| 122 | 42.8% | 64.9% | 34.2% | 22.8% | 73.4% | 27.4% | 56.4% | 42.3% | 39.3% | 86.0% | 35.5% | 64.5% | 41.9% | 34.7% | 86.5% |
| 123 | 32.3% | 68.1% | 44.1% | 39.7% | 76.9% | 13.5% | 70.4% | 55.7% | 49.7% | 89.3% | 18.4% | 75.1% | 57.0% | 45.2% | 89.8% |
| 124 | 20.6% | 63.3% | 40.5% | 22.0% | 74.9% | 10.1% | 62.9% | 54.2% | 53.0% | 88.4% | 14.1% | 68.7% | 53.6% | 42.6% | 88.6% |
| 125 | 20.7% | 62.2% | 36.1% | 31.6% | 73.4% | 7.8% | 54.0% | 48.6% | 44.7% | 86.6% | 14.9% | 65.8% | 46.1% | 42.1% | 88.4% |
| 126 | 21.9% | 65.6% | 32.5% | 24.2% | 75.7% | 8.0% | 62.1% | 46.1% | 43.3% | 88.9% | 12.1% | 70.2% | 46.4% | 39.1% | 91.3% |
| 127 | 37.7% | 54.7% | 29.7% | 19.3% | 65.5% | 33.1% | 63.6% | 47.2% | 29.1% | 86.9% | 38.5% | 61.8% | 36.3% | 23.2% | 86.9% |
| 128 | 26.9% | 60.6% | 30.8% | 25.0% | 67.8% | 9.9% | 71.3% | 42.6% | 35.1% | 87.4% | 17.2% | 72.2% | 30.0% | 19.6% | 87.1% |
| 129 | 22.2% | 57.5% | 36.0% | 16.1% | 68.9% | 10.2% | 64.9% | 50.9% | 30.7% | 87.3% | 15.7% | 55.3% | 37.0% | 18.7% | 86.9% |
| 130 | 18.0% | 59.6% | 28.6% | 29.3% | 68.5% | 6.4% | 67.1% | 41.2% | 37.8% | 88.1% | 11.6% | 61.8% | 31.9% | 30.6% | 87.2% |
| 131 | 22.8% | 58.8% | 29.2% | 18.6% | 69.1% | 10.1% | 72.8% | 55.8% | 31.2% | 89.9% | 18.9% | 68.4% | 44.2% | 24.9% | 89.3% |
| 132 | 37.7% | 46.3% | 20.9% | 11.7% | 73.0% | 24.9% | 38.4% | 23.4% | 18.4% | 85.6% | 21.4% | 36.1% | 19.5% | 13.6% | 83.6% |
| 133 | 18.9% | 67.9% | 18.6% | 8.8% | 75.2% | 6.3% | 69.6% | 25.7% | 13.9% | 87.0% | 9.1% | 69.1% | 17.8% | 10.2% | 85.4% |
| 134 | 15.9% | 65.6% | 32.2% | 6.9% | 82.8% | 7.6% | 50.9% | 46.2% | 25.8% | 91.1% | 12.5% | 46.3% | 36.1% | 15.6% | 91.3% |
| 135 | 11.8% | 64.8% | 17.6% | 12.0% | 74.4% | 6.0% | 50.4% | 32.2% | 32.6% | 85.8% | 11.4% | 50.7% | 24.9% | 23.9% | 85.7% |
| 136 | 12.7% | 70.0% | 17.4% | 6.1% | 83.6% | 6.1% | 64.2% | 47.6% | 30.4% | 92.5% | 9.3% | 49.9% | 26.9% | 16.0% | 92.1% |
| 137 | 39.9% | 53.3% | 21.5% | 11.0% | 75.0% | 24.5% | 48.1% | 35.9% | 26.6% | 86.9% | 26.6% | 45.2% | 27.9% | 18.9% | 88.2% |
| 138 | 14.6% | 58.3% | 40.3% | 9.7% | 70.3% | 8.4% | 58.0% | 47.4% | 27.5% | 85.4% | 11.1% | 49.3% | 36.8% | 17.2% | 83.7% |
| 139 | 10.7% | 56.6% | 17.0% | 16.3% | 76.2% | 4.5% | 53.9% | 28.5% | 25.4% | 85.4% | 7.5% | 42.4% | 17.3% | 16.8% | 83.2% |
| 140 | 10.7% | 61.0% | 17.5% | 7.0% | 84.1% | 9.1% | 69.9% | 43.5% | 18.9% | 89.3% | 8.8% | 57.9% | 26.1% | 12.3% | 88.9% |
| 141 | 46.2% | 58.8% | 25.8% | 18.5% | 77.8% | 36.3% | 56.4% | 38.1% | 23.4% | 88.1% | 27.9% | 45.2% | 23.2% | 15.0% | 87.0% |
| 142 | 12.4% | 66.7% | 25.2% | 17.8% | 73.5% | 8.4% | 68.6% | 28.6% | 16.9% | 83.7% | 9.5% | 61.8% | 18.0% | 11.7% | 82.2% |
| 143 | 14.3% | 53.7% | 37.8% | 12.7% | 68.9% | 8.7% | 54.5% | 39.5% | 20.4% | 83.0% | 10.5% | 40.5% | 24.3% | 13.1% | 78.5% |
| 144 | 11.7% | 58.2% | 27.7% | 32.0% | 71.7% | 7.7% | 63.9% | 30.6% | 27.5% | 86.3% | 11.7% | 48.9% | 20.9% | 20.4% | 83.9% |
| 145 | 15.8% | 51.3% | 24.1% | 18.1% | 72.0% | 10.4% | 62.8% | 34.8% | 21.8% | 87.0% | 12.9% | 52.7% | 24.2% | 15.9% | 84.5% |
| 146 | 35.8% | 44.6% | 18.0% | 18.3% | 72.9% | 35.3% | 57.3% | 41.1% | 23.7% | 88.3% | 24.9% | 38.9% | 19.7% | 12.6% | 82.0% |
| 147 | 18.1% | 75.6% | 24.1% | 29.4% | 81.9% | 11.9% | 76.2% | 40.5% | 21.3% | 88.2% | 10.3% | 73.7% | 20.8% | 11.0% | 87.9% |
| 148 | 15.9% | 50.8% | 35.3% | 15.5% | 78.9% | 16.0% | 68.5% | 58.0% | 27.8% | 90.2% | 12.8% | 48.6% | 33.3% | 14.7% | 88.6% |
| 149 | 14.5% | 56.3% | 23.2% | 37.7% | 78.2% | 4.6% | 63.9% | 30.7% | 27.2% | 87.6% | 9.0% | 50.3% | 19.4% | 20.1% | 86.7% |
| 150 | 18.7% | 58.3% | 23.1% | 17.9% | 76.5% | 7.3% | 64.1% | 44.8% | 16.2% | 89.9% | 11.6% | 58.1% | 29.3% | 14.5% | 89.0% |
| 151 | 42.2% | 54.5% | 18.6% | 20.1% | 74.1% | 39.2% | 60.2% | 38.0% | 23.5% | 86.2% | 31.1% | 51.7% | 26.9% | 16.8% | 85.4% |
| 152 | 27.7% | 72.0% | 19.3% | 21.3% | 77.4% | 16.8% | 75.0% | 35.4% | 29.2% | 87.5% | 15.3% | 76.7% | 28.4% | 17.2% | 89.0% |
| 153 | 19.2% | 58.5% | 32.4% | 11.5% | 74.2% | 11.9% | 62.4% | 48.0% | 22.1% | 86.5% | 13.3% | 54.9% | 37.0% | 14.5% | 84.5% |
| 154 | 16.2% | 69.1% | 18.8% | 35.5% | 83.1% | 6.9% | 71.5% | 32.7% | 30.5% | 90.7% | 8.4% | 65.3% | 24.7% | 24.5% | 90.9% |
| 155 | 22.0% | 61.5% | 17.3% | 16.7% | 85.3% | 17.8% | 71.7% | 48.4% | 20.0% | 91.5% | 12.6% | 61.4% | 32.8% | 14.8% | 91.5% |
| 156 | 43.8% | 49.2% | 20.1% | 16.0% | 68.8% | 42.6% | 62.5% | 39.7% | 20.9% | 86.7% | 37.3% | 54.2% | 27.1% | 16.8% | 86.5% |
| 157 | 30.4% | 58.2% | 21.6% | 17.6% | 67.5% | 9.0% | 65.6% | 31.7% | 21.6% | 85.2% | 17.2% | 66.5% | 25.5% | 15.9% | 85.5% |
| 158 | 21.1% | 62.4% | 32.8% | 9.7% | 80.7% | 13.1% | 68.8% | 59.6% | 25.6% | 89.0% | 11.3% | 53.3% | 41.0% | 13.3% | 87.5% |
| 159 | 19.2% | 71.1% | 20.8% | 32.9% | 82.7% | 6.2% | 69.9% | 33.6% | 31.6% | 90.9% | 10.0% | 63.6% | 23.2% | 23.4% | 90.8% |
| 160 | 33.1% | 56.9% | 21.9% | 16.3% | 73.8% | 8.1% | 67.7% | 44.7% | 22.4% | 89.3% | 16.4% | 60.2% | 32.0% | 16.4% | 88.4% |
| 161 | 39.8% | 50.1% | 19.8% | 13.2% | 64.5% | 43.6% | 63.8% | 46.5% | 24.4% | 86.3% | 35.5% | 52.7% | 27.8% | 17.4% | 83.3% |
| 162 | 35.0% | 63.4% | 19.4% | 15.6% | 71.0% | 25.0% | 72.0% | 37.1% | 27.7% | 86.2% | 23.8% | 70.3% | 24.7% | 14.3% | 85.1% |
| 163 | 18.6% | 47.5% | 24.6% | 11.7% | 68.9% | 12.1% | 64.1% | 48.5% | 22.6% | 83.6% | 13.7% | 51.0% | 30.4% | 13.3% | 82.5% |
| 164 | 28.2% | 58.2% | 18.3% | 20.0% | 74.2% | 6.0% | 67.5% | 31.1% | 24.5% | 88.3% | 14.3% | 51.1% | 21.6% | 17.4% | 85.5% |
| 165 | 29.8% | 55.3% | 20.2% | 14.8% | 70.6% | 11.1% | 64.5% | 42.1% | 16.3% | 85.6% | 14.9% | 61.0% | 29.9% | 13.0% | 85.4% |
| 166 | 28.5% | 58.5% | 23.1% | 17.7% | 65.9% | 20.1% | 65.4% | 36.3% | 25.9% | 83.1% | 18.1% | 64.0% | 25.7% | 15.6% | 81.1% |
| 167 | 21.3% | 52.1% | 25.1% | 12.1% | 64.3% | 13.3% | 61.6% | 44.1% | 24.6% | 83.8% | 16.7% | 53.7% | 31.9% | 16.1% | 83.5% |
| 168 | 18.1% | 60.1% | 21.5% | 18.8% | 67.3% | 6.1% | 68.1% | 35.2% | 26.8% | 86.4% | 13.0% | 60.4% | 25.5% | 19.8% | 83.0% |
| 169 | 21.2% | 54.7% | 20.3% | 13.0% | 65.5% | 17.8% | 65.6% | 43.7% | 17.9% | 83.5% | 17.7% | 58.9% | 29.0% | 14.7% | 81.9% |
| 170 | 36.4% | 55.7% | 29.9% | 14.9% | 64.2% | 27.6% | 59.7% | 40.8% | 20.8% | 83.9% | 32.5% | 59.8% | 34.0% | 17.5% | 84.0% |
| 171 | 32.5% | 58.5% | 33.4% | 19.2% | 65.8% | 10.7% | 62.3% | 36.7% | 26.3% | 82.7% | 19.4% | 63.2% | 27.7% | 17.2% | 80.7% |
| 172 | 27.9% | 57.3% | 37.3% | 12.7% | 65.7% | 10.7% | 64.2% | 51.5% | 26.4% | 84.2% | 22.0% | 59.2% | 37.7% | 17.2% | 84.6% |
| 173 | 21.5% | 56.8% | 24.6% | 17.5% | 65.4% | 6.7% | 57.7% | 30.0% | 22.8% | 81.8% | 14.0% | 56.5% | 24.9% | 19.9% | 80.9% |
| 174 | 26.3% | 56.2% | 33.9% | 14.0% | 63.7% | 9.3% | 65.9% | 52.0% | 23.6% | 84.7% | 16.8% | 62.3% | 34.1% | 15.6% | 82.2% |
| 175 | 41.1% | 60.1% | 38.8% | 15.6% | 66.8% | 25.7% | 56.0% | 39.8% | 24.7% | 81.0% | 36.1% | 59.4% | 34.3% | 19.0% | 80.5% |
| 176 | 33.5% | 56.2% | 30.6% | 16.3% | 62.9% | 9.5% | 66.0% | 42.0% | 33.1% | 84.1% | 20.7% | 62.7% | 26.9% | 16.2% | 80.0% |
| 177 | 23.3% | 55.0% | 34.9% | 13.5% | 65.5% | 11.8% | 61.0% | 48.5% | 32.2% | 85.2% | 19.1% | 58.1% | 40.3% | 18.8% | 85.0% |
| 178 | 18.0% | 54.2% | 26.2% | 19.1% | 61.8% | 6.9% | 59.3% | 35.5% | 28.9% | 80.6% | 12.9% | 54.9% | 27.2% | 22.2% | 79.7% |
| 179 | 28.6% | 61.5% | 37.1% | 15.8% | 69.6% | 8.1% | 65.8% | 49.4% | 27.1% | 85.9% | 19.0% | 64.6% | 38.9% | 18.7% | 86.7% |
| 180 | 42.9% | 60.9% | 44.5% | 20.6% | 67.1% | 27.6% | 60.6% | 45.8% | 30.1% | 83.7% | 37.7% | 62.9% | 39.2% | 23.1% | 83.4% |
| 181 | 36.7% | 64.7% | 45.1% | 25.6% | 71.1% | 11.0% | 67.9% | 46.6% | 40.8% | 86.0% | 22.0% | 68.6% | 35.0% | 21.8% | 84.5% |
| 182 | 20.3% | 52.9% | 38.7% | 15.7% | 63.2% | 8.9% | 57.8% | 44.4% | 32.5% | 82.4% | 16.0% | 55.0% | 35.4% | 19.3% | 79.6% |
| 183 | 17.4% | 57.1% | 30.5% | 20.5% | 64.5% | 6.3% | 58.1% | 36.0% | 31.8% | 81.5% | 15.0% | 57.4% | 30.9% | 25.6% | 81.4% |
| 184 | 24.7% | 57.5% | 37.4% | 17.6% | 65.6% | 6.6% | 62.8% | 46.7% | 28.1% | 82.3% | 15.7% | 60.2% | 36.2% | 18.8% | 79.6% |
| 185 | 39.7% | 58.2% | 41.4% | 23.5% | 65.7% | 27.9% | 58.4% | 38.4% | 33.9% | 82.6% | 36.8% | 63.0% | 39.7% | 27.5% | 83.0% |
| 186 | 19.5% | 57.3% | 35.9% | 25.9% | 65.2% | 5.3% | 60.9% | 37.8% | 33.1% | 82.7% | 15.3% | 59.5% | 34.0% | 30.2% | 82.5% |
| 187 | 44.1% | 60.9% | 44.7% | 29.6% | 67.5% | 22.9% | 58.9% | 41.2% | 32.3% | 84.1% | 35.2% | 60.5% | 35.2% | 24.1% | 83.1% |
| 188 | 37.0% | 64.7% | 47.6% | 40.8% | 71.4% | 10.4% | 65.3% | 38.1% | 36.6% | 87.1% | 22.4% | 65.9% | 38.9% | 29.4% | 85.8% |
| 189 | 25.3% | 58.4% | 43.9% | 25.8% | 66.4% | 12.5% | 65.5% | 49.2% | 33.1% | 85.5% | 19.8% | 62.6% | 40.2% | 22.2% | 85.1% |
| 190 | 18.6% | 55.4% | 35.5% | 27.0% | 63.7% | 6.2% | 54.1% | 36.6% | 35.4% | 79.9% | 15.8% | 58.1% | 33.9% | 29.3% | 79.0% |
| 191 | 22.7% | 58.4% | 37.4% | 21.0% | 66.6% | 9.2% | 66.8% | 47.0% | 35.8% | 86.6% | 17.0% | 64.9% | 38.4% | 24.0% | 85.3% |
| 192 | 41.7% | 58.9% | 38.8% | 29.3% | 66.3% | 28.4% | 58.4% | 38.1% | 33.9% | 86.2% | 34.3% | 62.7% | 38.2% | 25.3% | 84.7% |
| 193 | 36.1% | 60.7% | 45.3% | 38.9% | 68.4% | 10.0% | 69.7% | 39.8% | 37.4% | 85.9% | 19.8% | 66.7% | 32.0% | 25.8% | 83.4% |
| 194 | 28.7% | 63.4% | 47.7% | 25.9% | 70.5% | 9.8% | 66.2% | 54.1% | 31.4% | 89.9% | 15.8% | 68.2% | 44.4% | 21.9% | 88.2% |
| 195 | 34.4% | 72.7% | 55.4% | 51.1% | 76.9% | 4.7% | 64.1% | 39.9% | 40.1% | 87.8% | 19.9% | 65.5% | 35.0% | 34.7% | 84.7% |
| 196 | 36.9% | 68.3% | 49.9% | 34.6% | 73.9% | 9.2% | 75.9% | 58.8% | 38.7% | 90.6% | 18.5% | 67.9% | 41.6% | 25.2% | 86.1% |
| 197 | 40.7% | 59.6% | 40.5% | 26.7% | 66.0% | 24.4% | 53.7% | 34.6% | 31.6% | 84.0% | 33.2% | 62.2% | 37.8% | 24.1% | 84.8% |
| 198 | 35.4% | 59.6% | 43.6% | 36.7% | 67.6% | 10.6% | 65.8% | 42.4% | 34.0% | 85.9% | 22.7% | 66.6% | 41.7% | 32.6% | 87.6% |
| 199 | 27.7% | 55.1% | 38.8% | 23.5% | 63.7% | 6.8% | 56.5% | 45.0% | 37.3% | 85.6% | 19.5% | 59.2% | 37.0% | 22.6% | 81.9% |
| 200 | 28.4% | 60.3% | 41.1% | 36.8% | 67.5% | 8.7% | 52.7% | 33.8% | 35.2% | 83.3% | 18.2% | 60.7% | 32.1% | 29.7% | 81.9% |
| 201 | 25.0% | 57.7% | 40.7% | 26.3% | 65.0% | 6.7% | 62.2% | 37.3% | 30.3% | 85.3% | 15.5% | 62.7% | 37.2% | 26.9% | 82.6% |
| 202 | 38.7% | 58.7% | 40.4% | 25.0% | 66.1% | 28.1% | 55.7% | 37.1% | 36.8% | 86.7% | 31.9% | 64.6% | 37.7% | 25.8% | 85.7% |
| 203 | 28.1% | 59.7% | 45.5% | 35.0% | 66.4% | 9.3% | 63.9% | 35.8% | 28.9% | 86.2% | 18.6% | 67.1% | 37.5% | 27.6% | 87.9% |
| 204 | 21.6% | 56.7% | 40.5% | 20.6% | 65.6% | 7.3% | 54.2% | 39.8% | 27.6% | 84.7% | 13.6% | 59.5% | 34.8% | 19.7% | 83.0% |
| 205 | 18.2% | 55.6% | 33.6% | 29.1% | 62.2% | 5.2% | 49.5% | 26.9% | 28.8% | 81.1% | 12.4% | 56.3% | 26.9% | 25.0% | 77.5% |
| 206 | 28.4% | 67.6% | 51.8% | 30.2% | 73.5% | 4.4% | 68.1% | 44.9% | 33.7% | 88.8% | 13.9% | 68.5% | 41.7% | 24.4% | 86.7% |
| 207 | 46.3% | 66.4% | 51.6% | 28.5% | 72.5% | 27.5% | 51.4% | 35.1% | 39.0% | 88.1% | 39.1% | 68.0% | 40.8% | 24.5% | 86.6% |
| 208 | 26.4% | 63.2% | 48.6% | 40.3% | 69.2% | 9.4% | 67.4% | 33.3% | 35.0% | 85.3% | 16.8% | 68.1% | 36.7% | 32.5% | 84.9% |
| 209 | 19.2% | 55.5% | 41.6% | 21.4% | 63.9% | 5.9% | 51.3% | 38.3% | 22.3% | 82.7% | 12.0% | 56.0% | 33.9% | 17.5% | 78.0% |
| 210 | 20.7% | 59.2% | 36.1% | 30.5% | 66.7% | 6.3% | 58.8% | 34.5% | 34.2% | 85.6% | 13.1% | 61.5% | 29.7% | 25.5% | 83.0% |
| 211 | 17.9% | 66.2% | 50.4% | 28.7% | 72.1% | 5.8% | 66.9% | 41.3% | 36.8% | 88.9% | 13.9% | 69.5% | 41.6% | 27.8% | 88.2% |
| 212 | 38.8% | 58.6% | 39.5% | 23.9% | 66.4% | 22.0% | 53.5% | 36.9% | 33.9% | 86.1% | 31.3% | 64.3% | 36.0% | 23.4% | 85.5% |
| 213 | 23.9% | 56.4% | 42.7% | 33.6% | 62.6% | 7.0% | 61.2% | 25.7% | 24.0% | 81.0% | 12.9% | 58.0% | 26.4% | 19.4% | 77.3% |
| 214 | 21.8% | 62.1% | 40.4% | 32.4% | 70.1% | 5.7% | 53.2% | 30.3% | 33.0% | 87.3% | 14.4% | 62.8% | 33.2% | 29.9% | 86.7% |
| 215 | 18.6% | 62.9% | 46.6% | 25.9% | 69.3% | 6.8% | 64.0% | 41.1% | 30.4% | 86.9% | 12.8% | 64.1% | 37.2% | 22.4% | 83.8% |
| 216 | 35.7% | 59.5% | 42.7% | 25.2% | 66.3% | 22.4% | 46.1% | 28.1% | 27.7% | 84.0% | 33.2% | 59.4% | 32.9% | 24.0% | 84.1% |
| 217 | 24.1% | 59.0% | 45.4% | 35.6% | 66.3% | 8.1% | 70.0% | 34.8% | 30.8% | 85.5% | 14.0% | 64.7% | 30.2% | 24.2% | 80.9% |
| 218 | 18.3% | 55.2% | 40.6% | 20.4% | 64.2% | 9.1% | 45.8% | 33.2% | 24.0% | 83.6% | 15.6% | 56.8% | 35.5% | 18.8% | 81.2% |
| 219 | 16.3% | 56.9% | 35.9% | 30.6% | 64.3% | 4.5% | 47.9% | 27.6% | 28.9% | 83.2% | 13.2% | 57.7% | 29.3% | 27.7% | 82.9% |
| 220 | 18.4% | 59.8% | 43.6% | 23.7% | 67.1% | 6.6% | 63.2% | 41.5% | 31.3% | 84.4% | 11.4% | 62.3% | 34.5% | 21.8% | 80.3% |
| 221 | 40.9% | 61.3% | 43.6% | 29.1% | 68.1% | 29.3% | 53.3% | 35.4% | 34.1% | 87.7% | 37.2% | 60.3% | 36.4% | 26.0% | 87.3% |
| 222 | 23.0% | 60.2% | 44.9% | 33.3% | 67.1% | 9.3% | 67.5% | 33.9% | 28.7% | 84.2% | 16.7% | 66.3% | 31.8% | 21.8% | 82.8% |
| 223 | 17.5% | 58.3% | 33.8% | 28.2% | 66.3% | 5.9% | 56.3% | 32.5% | 32.4% | 84.7% | 12.5% | 59.6% | 30.7% | 30.2% | 86.5% |
| 224 | 37.0% | 58.4% | 37.8% | 24.7% | 66.8% | 30.2% | 59.7% | 40.5% | 36.4% | 88.7% | 36.7% | 61.1% | 36.3% | 27.7% | 89.4% |
| 225 | 32.3% | 71.2% | 54.3% | 42.1% | 76.5% | 11.1% | 82.0% | 39.9% | 42.8% | 92.3% | 16.2% | 84.4% | 32.2% | 31.2% | 92.7% |
| 226 | 21.0% | 58.7% | 43.8% | 21.8% | 66.4% | 7.5% | 55.2% | 42.2% | 28.1% | 84.8% | 13.1% | 54.9% | 35.3% | 18.4% | 83.3% |
| 227 | 17.7% | 56.0% | 33.7% | 27.9% | 64.8% | 6.3% | 57.3% | 35.0% | 35.8% | 85.5% | 11.8% | 55.2% | 30.4% | 34.1% | 84.9% |
| 228 | 20.5% | 59.1% | 39.3% | 23.7% | 67.2% | 7.6% | 70.1% | 46.9% | 37.4% | 89.6% | 11.5% | 69.3% | 40.9% | 31.9% | 89.0% |
| 229 | 50.1% | 63.7% | 35.1% | 21.1% | 70.5% | 36.2% | 55.1% | 38.6% | 29.2% | 89.7% | 43.5% | 52.4% | 30.7% | 22.7% | 88.1% |
| 230 | 27.6% | 58.6% | 37.0% | 24.9% | 65.9% | 13.8% | 69.1% | 39.4% | 34.1% | 86.6% | 18.1% | 69.9% | 29.9% | 21.2% | 86.2% |
| 231 | 21.4% | 58.5% | 41.4% | 17.7% | 67.6% | 6.4% | 59.6% | 47.2% | 32.8% | 86.2% | 14.0% | 55.8% | 38.2% | 23.0% | 85.5% |
| 232 | 18.5% | 59.2% | 32.6% | 28.5% | 66.7% | 7.8% | 62.6% | 38.3% | 38.7% | 87.1% | 14.4% | 58.4% | 31.3% | 33.2% | 85.9% |
| 233 | 20.3% | 60.4% | 38.0% | 21.0% | 67.6% | 7.9% | 69.5% | 46.3% | 35.8% | 89.5% | 11.8% | 64.8% | 38.3% | 26.4% | 87.8% |
| 234 | 48.5% | 61.6% | 32.0% | 18.9% | 67.9% | 21.3% | 45.4% | 28.9% | 20.8% | 85.0% | 36.1% | 49.3% | 27.1% | 17.8% | 83.5% |
| 235 | 42.9% | 71.2% | 44.1% | 32.6% | 76.2% | 6.9% | 80.6% | 40.1% | 29.5% | 91.0% | 15.2% | 79.5% | 27.4% | 16.8% | 89.3% |
| 236 | 24.1% | 58.4% | 39.6% | 16.2% | 66.8% | 14.1% | 64.1% | 50.1% | 30.8% | 86.0% | 17.2% | 59.8% | 41.0% | 18.5% | 85.2% |
| 237 | 28.7% | 65.4% | 37.7% | 21.2% | 72.5% | 8.6% | 76.0% | 58.4% | 38.2% | 91.4% | 16.4% | 73.0% | 45.1% | 25.3% | 91.2% |
| 238 | 46.1% | 63.0% | 35.3% | 19.3% | 70.3% | 33.3% | 62.5% | 46.1% | 34.8% | 88.9% | 40.4% | 63.0% | 37.9% | 23.4% | 88.2% |
| 239 | 26.8% | 60.6% | 41.4% | 15.6% | 68.5% | 8.3% | 64.9% | 55.5% | 37.2% | 88.2% | 13.5% | 60.0% | 42.3% | 22.8% | 85.7% |
| 240 | 23.9% | 59.5% | 28.2% | 25.0% | 66.8% | 6.0% | 63.6% | 40.1% | 38.3% | 87.3% | 16.8% | 61.1% | 31.4% | 28.3% | 86.1% |
| 241 | 25.7% | 57.6% | 32.1% | 17.9% | 65.2% | 8.4% | 71.9% | 55.6% | 34.0% | 87.9% | 19.2% | 66.2% | 44.3% | 24.6% | 84.8% |
| 242 | 48.4% | 66.0% | 37.5% | 22.2% | 73.1% | 34.4% | 61.5% | 44.7% | 35.3% | 90.6% | 46.0% | 67.8% | 46.0% | 30.7% | 91.9% |
| 243 | 37.9% | 61.8% | 33.8% | 26.5% | 68.7% | 7.9% | 68.0% | 36.6% | 28.5% | 84.7% | 26.1% | 72.2% | 35.0% | 24.1% | 86.4% |
| 244 | 26.3% | 56.5% | 36.3% | 15.3% | 64.8% | 13.1% | 69.1% | 57.5% | 41.4% | 88.7% | 16.5% | 62.1% | 44.8% | 22.0% | 86.3% |
| 245 | 22.5% | 62.3% | 31.6% | 30.6% | 69.1% | 5.3% | 59.5% | 33.9% | 32.9% | 85.8% | 10.8% | 57.8% | 28.3% | 28.4% | 85.2% |
| 246 | 30.1% | 64.4% | 34.7% | 18.8% | 71.0% | 8.3% | 73.7% | 55.7% | 32.9% | 88.2% | 15.9% | 70.8% | 46.1% | 23.0% | 88.6% |
| 247 | 41.3% | 57.6% | 29.1% | 20.2% | 67.5% | 33.2% | 64.8% | 47.2% | 29.3% | 87.4% | 42.3% | 59.2% | 33.5% | 20.8% | 86.2% |
| 248 | 30.1% | 62.5% | 30.0% | 25.6% | 68.8% | 14.8% | 75.3% | 44.6% | 37.4% | 88.1% | 20.3% | 72.2% | 28.4% | 20.0% | 85.6% |
| 249 | 22.8% | 57.2% | 36.5% | 15.1% | 69.0% | 10.3% | 70.4% | 57.3% | 35.6% | 89.2% | 16.5% | 60.3% | 42.2% | 19.6% | 88.6% |
| 250 | 21.8% | 60.0% | 30.5% | 29.9% | 70.1% | 4.8% | 72.7% | 42.6% | 36.1% | 88.2% | 16.4% | 64.8% | 33.2% | 28.0% | 86.8% |
| 251 | 23.0% | 63.6% | 30.9% | 19.6% | 73.3% | 7.0% | 77.0% | 60.1% | 28.4% | 89.7% | 19.3% | 63.7% | 37.6% | 18.0% | 88.9% |
| 252 | 53.6% | 68.5% | 16.5% | 5.0% | 88.3% | 20.0% | 34.5% | 27.0% | 18.0% | 92.8% | 25.2% | 35.5% | 19.3% | 12.5% | 93.5% |
| 253 | 16.3% | 73.7% | 17.5% | 9.0% | 79.2% | 5.2% | 73.6% | 27.3% | 17.2% | 88.4% | 9.7% | 73.0% | 18.8% | 10.1% | 88.4% |
| 254 | 10.2% | 62.0% | 29.4% | 4.3% | 88.9% | 5.6% | 48.1% | 46.6% | 21.5% | 92.8% | 6.7% | 33.2% | 27.4% | 9.9% | 91.9% |
| 255 | 12.8% | 63.2% | 14.8% | 10.7% | 79.4% | 4.2% | 44.3% | 22.9% | 24.0% | 83.7% | 9.4% | 43.6% | 20.1% | 19.9% | 85.7% |
| 256 | 11.7% | 68.0% | 15.2% | 6.2% | 87.3% | 3.7% | 58.2% | 36.5% | 15.9% | 92.4% | 7.3% | 42.9% | 21.2% | 11.1% | 91.5% |
| 257 | 41.8% | 62.5% | 22.0% | 9.8% | 74.5% | 15.8% | 50.4% | 33.7% | 20.1% | 85.6% | 21.0% | 44.5% | 22.2% | 13.9% | 85.9% |
| 258 | 22.0% | 64.4% | 18.7% | 10.8% | 70.4% | 7.3% | 68.4% | 40.9% | 24.4% | 86.0% | 13.2% | 70.3% | 28.7% | 15.9% | 85.6% |
| 259 | 18.4% | 69.8% | 33.0% | 6.3% | 81.3% | 4.9% | 57.1% | 43.0% | 15.7% | 87.4% | 9.0% | 43.9% | 28.5% | 10.6% | 87.3% |
| 260 | 11.3% | 75.6% | 13.9% | 11.4% | 87.7% | 3.7% | 51.5% | 21.5% | 24.8% | 89.4% | 7.2% | 44.9% | 15.2% | 17.7% | 91.4% |
| 261 | 15.4% | 63.8% | 17.3% | 7.9% | 79.4% | 6.7% | 65.5% | 45.0% | 23.8% | 87.8% | 9.2% | 58.7% | 33.1% | 17.1% | 88.8% |
| 262 | 25.1% | 60.0% | 26.5% | 14.9% | 73.3% | 27.9% | 49.9% | 34.4% | 18.5% | 83.4% | 20.0% | 41.4% | 21.8% | 13.8% | 84.5% |
| 263 | 19.7% | 62.1% | 19.0% | 15.9% | 71.0% | 10.2% | 66.7% | 28.3% | 20.0% | 84.1% | 14.0% | 65.6% | 21.0% | 14.1% | 83.8% |
| 264 | 13.2% | 70.9% | 44.3% | 10.3% | 80.0% | 5.4% | 52.7% | 42.1% | 13.7% | 86.1% | 12.4% | 42.9% | 26.7% | 12.3% | 85.3% |
| 265 | 12.0% | 67.3% | 30.9% | 33.1% | 77.3% | 4.8% | 64.4% | 34.8% | 27.0% | 86.2% | 10.1% | 52.8% | 21.8% | 20.6% | 85.5% |
| 266 | 14.7% | 63.2% | 33.8% | 22.7% | 76.9% | 9.2% | 70.7% | 50.1% | 29.9% | 86.3% | 10.3% | 57.5% | 32.5% | 20.9% | 85.0% |
| 267 | 39.7% | 53.6% | 23.6% | 23.7% | 70.9% | 35.7% | 50.9% | 35.2% | 23.9% | 83.7% | 29.8% | 40.8% | 23.8% | 16.1% | 80.1% |
| 268 | 21.4% | 67.5% | 18.5% | 18.6% | 77.9% | 6.4% | 76.2% | 35.2% | 20.8% | 89.2% | 12.9% | 71.1% | 22.4% | 12.6% | 86.8% |
| 269 | 15.9% | 56.5% | 42.9% | 17.2% | 74.6% | 8.2% | 56.0% | 43.2% | 16.3% | 85.0% | 12.2% | 45.7% | 29.7% | 13.4% | 84.9% |
| 270 | 14.1% | 63.1% | 27.0% | 44.3% | 83.1% | 9.4% | 73.5% | 39.9% | 33.5% | 92.0% | 10.3% | 59.4% | 26.5% | 26.1% | 93.1% |
| 271 | 19.1% | 58.6% | 32.6% | 23.2% | 75.0% | 6.8% | 70.6% | 51.7% | 20.8% | 87.0% | 11.8% | 51.6% | 25.5% | 13.6% | 82.6% |
| 272 | 47.1% | 43.3% | 16.7% | 15.8% | 72.8% | 37.3% | 59.4% | 41.4% | 23.2% | 87.0% | 35.6% | 44.2% | 24.3% | 14.2% | 83.2% |
| 273 | 27.5% | 63.5% | 20.5% | 20.7% | 72.3% | 10.0% | 67.1% | 36.5% | 24.6% | 84.8% | 15.7% | 69.5% | 27.2% | 15.4% | 85.1% |
| 274 | 24.1% | 48.6% | 23.9% | 9.3% | 75.0% | 7.6% | 63.8% | 51.2% | 21.2% | 87.7% | 13.5% | 43.6% | 29.7% | 11.7% | 84.7% |
| 275 | 16.2% | 61.7% | 22.3% | 33.7% | 75.5% | 6.7% | 61.9% | 29.8% | 25.0% | 85.7% | 11.7% | 56.3% | 22.0% | 19.9% | 86.3% |
| 276 | 21.7% | 62.8% | 24.2% | 19.0% | 75.6% | 8.4% | 69.1% | 46.2% | 18.9% | 86.3% | 11.7% | 65.2% | 34.8% | 14.2% | 85.2% |
| 277 | 41.2% | 58.4% | 25.0% | 17.9% | 69.5% | 34.3% | 60.0% | 41.1% | 25.0% | 83.0% | 35.5% | 60.5% | 34.5% | 19.8% | 86.2% |
| 278 | 36.8% | 64.1% | 18.4% | 17.0% | 71.4% | 28.5% | 75.1% | 41.5% | 28.6% | 87.9% | 25.0% | 72.6% | 28.4% | 17.3% | 86.6% |
| 279 | 21.1% | 67.3% | 20.0% | 33.7% | 82.9% | 7.0% | 74.8% | 30.2% | 26.2% | 91.4% | 10.5% | 68.0% | 24.7% | 22.4% | 90.3% |
| 280 | 33.7% | 64.5% | 21.6% | 16.5% | 82.2% | 7.6% | 78.0% | 53.9% | 21.3% | 93.5% | 13.3% | 67.0% | 36.2% | 14.1% | 92.3% |
| 281 | 43.6% | 59.4% | 23.2% | 14.0% | 68.3% | 30.1% | 60.1% | 37.9% | 20.2% | 86.3% | 34.2% | 54.9% | 27.8% | 17.0% | 85.0% |
| 282 | 40.5% | 65.3% | 21.8% | 17.0% | 72.1% | 7.5% | 69.5% | 31.6% | 18.4% | 85.5% | 18.3% | 72.9% | 26.2% | 14.3% | 87.9% |
| 283 | 27.3% | 52.2% | 23.6% | 11.2% | 68.5% | 13.0% | 65.8% | 50.7% | 25.3% | 85.3% | 17.6% | 54.1% | 33.6% | 15.2% | 83.2% |
| 284 | 30.4% | 55.0% | 17.1% | 19.2% | 70.2% | 5.1% | 66.7% | 30.9% | 22.0% | 86.7% | 15.7% | 50.9% | 20.6% | 15.8% | 82.6% |
| 285 | 31.1% | 60.0% | 23.5% | 15.3% | 70.1% | 16.5% | 71.5% | 57.6% | 27.5% | 87.8% | 21.7% | 60.6% | 33.4% | 17.1% | 85.2% |
| 286 | 43.2% | 58.8% | 24.1% | 14.4% | 67.0% | 41.2% | 63.4% | 45.9% | 20.7% | 82.6% | 40.4% | 58.9% | 32.3% | 16.9% | 81.8% |
| 287 | 40.0% | 63.0% | 31.7% | 20.5% | 68.8% | 7.7% | 68.3% | 41.8% | 27.2% | 85.5% | 20.8% | 68.0% | 27.6% | 16.4% | 83.7% |
| 288 | 30.1% | 55.5% | 29.0% | 12.5% | 65.4% | 8.1% | 61.0% | 45.3% | 21.1% | 81.2% | 15.6% | 52.6% | 34.5% | 14.9% | 77.1% |
| 289 | 26.4% | 62.9% | 22.3% | 19.9% | 70.4% | 4.8% | 65.8% | 34.2% | 24.0% | 83.4% | 16.0% | 59.0% | 26.5% | 19.9% | 84.7% |
| 290 | 28.9% | 59.7% | 26.6% | 17.2% | 67.7% | 7.9% | 62.8% | 42.6% | 18.4% | 82.6% | 15.1% | 63.6% | 35.4% | 15.8% | 83.3% |
| 291 | 52.8% | 67.0% | 31.1% | 15.3% | 73.6% | 23.0% | 55.1% | 38.5% | 18.9% | 88.4% | 40.9% | 53.5% | 29.7% | 14.7% | 88.6% |
| 292 | 46.8% | 68.6% | 31.0% | 18.4% | 74.2% | 7.9% | 68.9% | 40.3% | 25.8% | 86.2% | 22.7% | 71.1% | 28.4% | 15.4% | 86.9% |
| 293 | 29.1% | 56.4% | 35.0% | 13.6% | 65.0% | 15.6% | 63.1% | 48.4% | 27.6% | 84.8% | 20.8% | 57.4% | 36.8% | 17.1% | 81.9% |
| 294 | 23.7% | 60.3% | 28.1% | 20.4% | 67.5% | 6.8% | 59.2% | 29.1% | 21.4% | 81.2% | 15.9% | 58.9% | 26.0% | 20.0% | 81.9% |
| 295 | 29.2% | 64.3% | 36.8% | 15.5% | 71.6% | 9.1% | 70.2% | 54.8% | 26.2% | 86.2% | 16.6% | 68.0% | 39.5% | 17.5% | 86.5% |
| 296 | 39.1% | 54.5% | 32.2% | 14.3% | 61.5% | 20.3% | 57.4% | 44.1% | 25.8% | 78.2% | 34.6% | 55.7% | 32.0% | 17.4% | 76.5% |
| 297 | 34.9% | 58.2% | 41.2% | 24.6% | 64.5% | 6.2% | 64.6% | 43.8% | 35.9% | 81.8% | 21.2% | 64.3% | 35.1% | 20.9% | 81.5% |
| 298 | 25.5% | 56.5% | 41.1% | 15.5% | 65.6% | 8.5% | 59.8% | 44.4% | 27.6% | 82.6% | 18.4% | 59.1% | 39.0% | 17.9% | 81.9% |
| 299 | 27.6% | 60.1% | 39.3% | 18.8% | 67.3% | 6.4% | 64.4% | 47.9% | 28.4% | 84.6% | 17.4% | 64.3% | 39.7% | 21.1% | 84.7% |
| 300 | 42.1% | 61.8% | 41.6% | 18.9% | 68.7% | 13.2% | 55.4% | 38.0% | 22.8% | 83.9% | 35.1% | 60.6% | 34.9% | 19.1% | 84.2% |
| 301 | 34.1% | 58.7% | 44.6% | 33.3% | 65.1% | 6.8% | 65.7% | 44.1% | 33.3% | 81.9% | 19.4% | 61.7% | 32.1% | 19.8% | 77.4% |
| 302 | 25.9% | 57.5% | 41.8% | 16.5% | 66.1% | 7.3% | 59.4% | 44.4% | 29.4% | 83.4% | 17.8% | 60.7% | 38.8% | 18.9% | 83.3% |
| 303 | 20.9% | 61.7% | 34.9% | 24.3% | 68.5% | 5.5% | 59.4% | 36.0% | 28.4% | 82.1% | 15.3% | 62.7% | 34.8% | 27.4% | 82.4% |
| 304 | 23.8% | 60.5% | 34.4% | 14.6% | 68.6% | 6.6% | 66.5% | 52.9% | 35.5% | 84.1% | 14.6% | 64.8% | 42.3% | 22.1% | 83.5% |
| 305 | 44.8% | 60.8% | 44.7% | 28.5% | 67.6% | 23.5% | 58.2% | 41.6% | 32.5% | 83.6% | 38.7% | 60.1% | 38.8% | 24.8% | 80.9% |
| 306 | 38.7% | 58.9% | 42.5% | 32.2% | 65.8% | 5.6% | 63.8% | 42.4% | 36.2% | 83.4% | 24.2% | 63.8% | 33.8% | 22.7% | 81.7% |
| 307 | 27.4% | 54.7% | 37.5% | 15.0% | 63.3% | 6.1% | 56.5% | 38.8% | 29.6% | 81.1% | 19.9% | 57.0% | 37.4% | 20.4% | 80.3% |
| 308 | 23.3% | 56.9% | 34.7% | 26.8% | 66.0% | 5.1% | 61.3% | 33.5% | 26.3% | 82.0% | 16.7% | 60.6% | 33.8% | 26.1% | 82.1% |
| 309 | 30.4% | 63.1% | 46.3% | 19.5% | 69.8% | 5.4% | 63.0% | 47.0% | 27.2% | 84.3% | 18.9% | 65.8% | 42.5% | 22.5% | 84.6% |
| 310 | 40.5% | 57.4% | 41.6% | 26.2% | 64.1% | 26.3% | 61.0% | 42.0% | 31.3% | 82.4% | 37.1% | 62.4% | 38.0% | 23.6% | 82.4% |
| 311 | 39.2% | 63.5% | 51.6% | 41.7% | 70.2% | 8.6% | 67.8% | 38.4% | 38.9% | 83.2% | 24.2% | 65.2% | 32.4% | 23.8% | 82.0% |
| 312 | 22.7% | 54.0% | 39.7% | 18.6% | 62.8% | 9.4% | 61.8% | 45.6% | 39.4% | 86.0% | 20.7% | 63.2% | 41.3% | 25.4% | 83.2% |
| 313 | 25.2% | 64.0% | 45.5% | 37.6% | 71.3% | 5.1% | 63.6% | 38.2% | 34.9% | 85.4% | 19.4% | 60.3% | 34.9% | 29.0% | 83.5% |
| 314 | 22.3% | 60.3% | 42.2% | 19.6% | 67.7% | 5.4% | 68.8% | 49.2% | 33.0% | 86.1% | 15.6% | 60.8% | 34.9% | 21.2% | 82.9% |
| 315 | 44.3% | 60.4% | 41.2% | 28.2% | 67.0% | 26.3% | 68.7% | 51.8% | 36.2% | 87.1% | 42.0% | 65.1% | 40.9% | 27.7% | 84.2% |
| 316 | 34.2% | 57.0% | 41.9% | 35.4% | 63.6% | 12.5% | 65.2% | 35.3% | 29.3% | 82.5% | 23.6% | 62.1% | 30.5% | 23.4% | 79.9% |
| 317 | 26.3% | 60.9% | 42.4% | 21.7% | 67.9% | 7.6% | 66.6% | 52.4% | 34.0% | 88.4% | 19.7% | 62.1% | 38.6% | 20.4% | 85.7% |
| 318 | 33.1% | 60.9% | 43.8% | 39.2% | 67.5% | 4.3% | 64.9% | 37.0% | 35.3% | 84.2% | 21.4% | 57.6% | 30.6% | 27.7% | 78.9% |
| 319 | 30.0% | 60.5% | 43.7% | 28.8% | 68.0% | 7.4% | 69.9% | 52.7% | 36.4% | 86.7% | 20.6% | 62.7% | 36.3% | 22.0% | 82.7% |
| 320 | 33.5% | 59.6% | 43.7% | 36.2% | 66.7% | 5.9% | 66.6% | 38.4% | 35.1% | 85.1% | 20.6% | 65.0% | 33.1% | 26.3% | 83.9% |
| 321 | 33.4% | 63.6% | 49.6% | 30.4% | 71.0% | 7.2% | 67.4% | 53.7% | 43.5% | 89.4% | 21.8% | 64.5% | 41.4% | 23.8% | 86.3% |
| 322 | 28.1% | 61.1% | 40.4% | 36.0% | 68.8% | 5.1% | 58.5% | 36.3% | 33.9% | 85.6% | 21.2% | 62.5% | 28.9% | 25.9% | 83.5% |
| 323 | 35.2% | 66.9% | 47.7% | 29.2% | 73.7% | 4.8% | 71.1% | 49.5% | 33.3% | 87.6% | 19.8% | 66.3% | 37.1% | 22.4% | 85.5% |
| 324 | 48.4% | 64.6% | 47.2% | 29.3% | 71.0% | 18.7% | 64.9% | 45.7% | 37.2% | 87.5% | 36.7% | 65.3% | 39.7% | 25.7% | 86.2% |
| 325 | 33.1% | 62.1% | 46.3% | 36.2% | 69.5% | 7.4% | 70.5% | 37.3% | 34.7% | 85.8% | 18.1% | 68.8% | 34.0% | 24.1% | 86.0% |
| 326 | 30.5% | 64.7% | 50.0% | 26.5% | 71.0% | 8.5% | 69.2% | 54.9% | 46.9% | 88.6% | 19.4% | 67.9% | 46.1% | 26.1% | 87.7% |
| 327 | 25.0% | 61.7% | 42.5% | 24.3% | 69.7% | 6.7% | 67.2% | 45.8% | 28.7% | 86.8% | 15.6% | 66.3% | 38.4% | 23.1% | 85.8% |
| 328 | 33.2% | 67.5% | 52.8% | 42.1% | 73.3% | 5.4% | 70.4% | 35.0% | 29.6% | 86.6% | 14.0% | 69.0% | 31.9% | 21.5% | 85.1% |
| 329 | 25.3% | 65.9% | 53.4% | 25.6% | 73.1% | 7.6% | 67.9% | 53.7% | 32.9% | 88.6% | 12.8% | 64.1% | 44.2% | 18.7% | 86.1% |
| 330 | 21.7% | 62.1% | 40.7% | 33.5% | 69.9% | 5.1% | 57.7% | 30.1% | 32.1% | 86.0% | 14.3% | 63.6% | 29.4% | 25.5% | 84.4% |
| 331 | 21.1% | 60.7% | 44.7% | 23.9% | 67.2% | 6.4% | 66.5% | 45.6% | 29.1% | 87.0% | 13.6% | 64.8% | 38.6% | 21.6% | 84.8% |
| 332 | 39.8% | 59.5% | 43.3% | 26.8% | 67.5% | 31.9% | 56.8% | 38.0% | 29.3% | 88.6% | 33.6% | 62.7% | 35.7% | 20.0% | 85.1% |
| 333 | 27.9% | 62.4% | 49.0% | 38.6% | 69.0% | 5.7% | 69.6% | 33.7% | 30.0% | 85.3% | 14.7% | 67.6% | 34.0% | 24.0% | 84.2% |
| 334 | 20.6% | 58.4% | 43.3% | 18.9% | 67.2% | 11.4% | 63.7% | 48.8% | 37.1% | 88.3% | 15.9% | 62.9% | 41.9% | 21.5% | 83.7% |
| 335 | 19.9% | 60.8% | 39.7% | 33.3% | 68.5% | 4.1% | 53.2% | 29.8% | 26.7% | 84.9% | 12.7% | 62.8% | 32.8% | 27.8% | 85.6% |
| 336 | 21.8% | 66.2% | 51.1% | 33.4% | 72.6% | 4.1% | 69.7% | 49.1% | 29.7% | 87.6% | 10.5% | 66.7% | 39.6% | 24.2% | 84.6% |
| 337 | 37.2% | 58.1% | 40.2% | 27.9% | 66.1% | 21.2% | 53.1% | 33.2% | 37.0% | 85.3% | 30.6% | 63.2% | 39.6% | 30.0% | 84.9% |
| 338 | 25.2% | 59.2% | 44.9% | 35.3% | 66.2% | 5.4% | 64.0% | 24.3% | 17.9% | 81.1% | 14.0% | 64.8% | 27.9% | 18.5% | 81.4% |
| 339 | 18.9% | 59.2% | 45.1% | 23.2% | 66.8% | 6.2% | 54.8% | 40.5% | 21.8% | 85.8% | 13.0% | 60.1% | 41.3% | 18.4% | 85.8% |
| 340 | 17.3% | 56.5% | 36.0% | 28.6% | 62.8% | 5.0% | 56.1% | 29.8% | 28.6% | 84.5% | 11.1% | 61.0% | 29.4% | 25.7% | 82.0% |
| 341 | 18.3% | 64.0% | 42.4% | 21.3% | 71.3% | 10.4% | 73.7% | 50.5% | 37.8% | 90.0% | 15.1% | 73.0% | 45.1% | 25.0% | 88.0% |
| 342 | 42.2% | 64.7% | 47.5% | 30.5% | 70.7% | 11.8% | 43.5% | 28.1% | 23.0% | 88.6% | 25.7% | 61.9% | 34.5% | 20.4% | 89.8% |
| 343 | 29.6% | 62.0% | 46.0% | 34.7% | 68.3% | 10.8% | 73.1% | 35.2% | 33.6% | 86.6% | 16.7% | 73.9% | 33.9% | 26.8% | 86.6% |
| 344 | 22.3% | 57.8% | 42.3% | 18.6% | 65.4% | 10.9% | 55.1% | 39.7% | 25.5% | 84.7% | 15.7% | 58.8% | 38.7% | 17.5% | 84.2% |
| 345 | 15.6% | 56.7% | 35.8% | 31.4% | 64.0% | 5.5% | 55.2% | 32.8% | 33.4% | 86.2% | 12.9% | 60.4% | 32.1% | 32.1% | 84.9% |
| 346 | 23.9% | 62.6% | 43.8% | 25.8% | 70.2% | 5.5% | 69.4% | 45.4% | 29.1% | 90.4% | 12.9% | 67.2% | 37.3% | 21.3% | 88.5% |
| 347 | 41.8% | 60.2% | 37.3% | 21.3% | 67.3% | 34.1% | 61.2% | 44.6% | 31.8% | 85.0% | 40.3% | 62.7% | 37.7% | 24.4% | 86.9% |
| 348 | 34.3% | 64.3% | 45.0% | 36.3% | 70.4% | 6.8% | 74.4% | 44.6% | 40.7% | 87.8% | 15.5% | 72.6% | 30.5% | 22.8% | 86.0% |
| 349 | 23.2% | 59.4% | 41.1% | 17.4% | 68.8% | 11.7% | 65.8% | 51.4% | 35.9% | 86.8% | 18.2% | 63.3% | 45.5% | 22.6% | 87.4% |
| 350 | 20.2% | 60.7% | 35.5% | 31.9% | 68.1% | 4.6% | 59.3% | 32.9% | 31.9% | 84.7% | 13.1% | 58.4% | 30.1% | 33.4% | 86.1% |
| 351 | 21.5% | 64.5% | 44.5% | 24.5% | 70.8% | 7.0% | 71.5% | 49.8% | 32.4% | 88.7% | 13.9% | 67.6% | 39.6% | 23.3% | 88.9% |
| 352 | 55.5% | 69.3% | 42.4% | 25.4% | 74.4% | 21.8% | 54.3% | 37.6% | 28.4% | 90.7% | 41.5% | 51.2% | 29.8% | 21.2% | 90.7% |
| 353 | 33.5% | 60.6% | 43.9% | 33.6% | 66.0% | 5.6% | 72.3% | 39.7% | 34.8% | 86.1% | 16.5% | 71.9% | 30.2% | 21.8% | 83.6% |
| 354 | 17.8% | 58.4% | 33.6% | 29.5% | 67.4% | 8.1% | 63.3% | 44.5% | 41.3% | 89.5% | 14.3% | 67.0% | 40.2% | 38.5% | 90.9% |
| 355 | 22.8% | 60.5% | 36.4% | 20.8% | 68.0% | 7.6% | 69.8% | 51.8% | 33.3% | 87.4% | 13.9% | 63.7% | 37.6% | 22.5% | 84.2% |
| 356 | 52.7% | 67.2% | 40.9% | 23.0% | 73.3% | 23.8% | 66.6% | 52.0% | 38.7% | 93.1% | 54.1% | 68.2% | 43.7% | 27.1% | 92.8% |
| 357 | 41.8% | 69.1% | 38.7% | 30.2% | 74.4% | 9.6% | 87.9% | 49.9% | 45.2% | 94.4% | 18.8% | 85.3% | 32.8% | 23.4% | 92.7% |
| 358 | 32.7% | 72.2% | 54.9% | 15.9% | 77.4% | 7.4% | 59.6% | 51.6% | 24.9% | 88.9% | 14.7% | 52.0% | 40.8% | 16.3% | 86.9% |
| 359 | 25.4% | 71.8% | 35.2% | 36.1% | 77.2% | 4.3% | 52.6% | 27.0% | 27.2% | 85.8% | 11.9% | 50.4% | 21.6% | 23.8% | 84.5% |
| 360 | 27.2% | 61.9% | 35.8% | 18.9% | 68.4% | 7.9% | 72.1% | 55.6% | 33.4% | 88.2% | 17.6% | 67.4% | 40.0% | 22.5% | 86.5% |
| 361 | 46.9% | 63.4% | 34.6% | 18.8% | 69.4% | 40.4% | 68.5% | 52.8% | 39.1% | 91.7% | 44.4% | 64.5% | 41.4% | 22.8% | 86.7% |
| 362 | 35.7% | 67.7% | 48.1% | 16.1% | 75.1% | 19.1% | 75.1% | 64.9% | 42.4% | 91.7% | 24.5% | 69.4% | 54.9% | 24.4% | 89.6% |
| 363 | 23.9% | 63.6% | 33.7% | 30.3% | 71.3% | 6.4% | 70.1% | 45.7% | 40.6% | 89.5% | 16.6% | 64.4% | 36.0% | 33.6% | 86.8% |
| 364 | 39.3% | 71.3% | 36.7% | 19.5% | 77.7% | 4.3% | 78.5% | 60.7% | 29.7% | 91.1% | 24.5% | 70.3% | 46.1% | 20.1% | 89.4% |
| 365 | 50.6% | 62.5% | 35.9% | 20.2% | 68.9% | 45.1% | 66.1% | 47.7% | 34.6% | 87.2% | 52.8% | 71.2% | 49.1% | 30.2% | 88.0% |
| 366 | 41.2% | 66.9% | 36.5% | 31.1% | 72.6% | 4.8% | 71.3% | 34.6% | 24.9% | 86.6% | 17.4% | 78.1% | 29.0% | 17.7% | 89.9% |
| 367 | 33.2% | 69.1% | 48.9% | 18.5% | 75.0% | 16.3% | 72.1% | 59.8% | 34.8% | 89.7% | 18.6% | 67.3% | 52.6% | 23.3% | 90.1% |
| 368 | 26.7% | 68.4% | 36.3% | 35.8% | 74.5% | 3.3% | 64.9% | 35.3% | 35.8% | 90.0% | 11.2% | 60.0% | 27.6% | 31.0% | 90.6% |
| 369 | 37.8% | 70.3% | 37.8% | 19.5% | 76.5% | 4.7% | 77.4% | 63.0% | 32.3% | 89.6% | 24.3% | 74.6% | 52.0% | 21.9% | 90.8% |
| 370 | 46.9% | 62.4% | 32.4% | 20.0% | 73.0% | 29.1% | 60.7% | 43.6% | 33.9% | 90.3% | 44.5% | 56.0% | 34.8% | 23.3% | 89.9% |
| 371 | 35.5% | 68.3% | 35.1% | 26.3% | 74.9% | 15.5% | 76.6% | 53.0% | 46.9% | 89.1% | 21.7% | 75.6% | 40.2% | 28.7% | 87.8% |
| 372 | 24.4% | 58.2% | 35.7% | 15.0% | 68.7% | 16.5% | 74.0% | 62.7% | 45.6% | 89.4% | 20.0% | 63.8% | 47.4% | 25.3% | 86.6% |
| 373 | 22.9% | 62.1% | 30.6% | 30.2% | 70.5% | 5.0% | 73.0% | 49.2% | 44.0% | 90.3% | 17.2% | 63.3% | 33.6% | 32.0% | 88.1% |
| 374 | 26.2% | 62.7% | 31.4% | 20.8% | 72.3% | 8.4% | 77.8% | 65.8% | 37.5% | 91.0% | 20.2% | 68.1% | 46.2% | 22.9% | 88.9% |
| 375 | 55.0% | 73.2% | 18.8% | 5.5% | 87.3% | 26.5% | 48.6% | 36.2% | 23.7% | 91.9% | 36.8% | 45.7% | 28.0% | 16.0% | 92.7% |
| 376 | 28.2% | 72.4% | 22.3% | 10.3% | 78.0% | 5.4% | 63.8% | 21.5% | 14.4% | 83.4% | 12.9% | 66.9% | 22.6% | 16.0% | 84.1% |
| 377 | 15.8% | 64.0% | 31.9% | 7.9% | 76.9% | 4.8% | 45.5% | 34.7% | 16.9% | 83.6% | 10.5% | 43.3% | 29.7% | 12.4% | 85.0% |
| 378 | 13.0% | 66.9% | 14.9% | 10.2% | 80.1% | 4.3% | 43.3% | 25.4% | 29.5% | 85.8% | 9.4% | 42.5% | 21.0% | 22.5% | 87.5% |
| 379 | 20.5% | 58.9% | 21.6% | 9.6% | 75.7% | 5.3% | 52.0% | 37.8% | 26.8% | 87.3% | 12.6% | 58.3% | 42.7% | 26.9% | 87.9% |
| 380 | 42.0% | 59.8% | 26.1% | 11.9% | 80.1% | 24.6% | 70.5% | 42.0% | 37.7% | 89.3% | 30.5% | 63.3% | 32.8% | 27.9% | 89.4% |
| 381 | 21.8% | 72.0% | 27.0% | 12.5% | 78.1% | 7.0% | 68.8% | 29.1% | 18.1% | 85.7% | 11.9% | 70.7% | 21.9% | 12.8% | 86.1% |
| 382 | 19.5% | 71.9% | 31.9% | 6.2% | 84.0% | 12.0% | 64.4% | 52.9% | 22.0% | 88.0% | 14.3% | 55.1% | 41.8% | 15.3% | 90.2% |
| 383 | 10.6% | 74.2% | 12.4% | 10.1% | 88.0% | 3.6% | 67.5% | 30.5% | 24.6% | 89.5% | 7.6% | 58.8% | 20.3% | 19.2% | 90.3% |
| 384 | 41.1% | 68.8% | 34.1% | 15.5% | 78.1% | 25.4% | 60.2% | 38.5% | 19.3% | 88.0% | 23.8% | 47.6% | 23.1% | 13.2% | 86.6% |
| 385 | 24.8% | 66.8% | 21.8% | 17.3% | 74.0% | 5.8% | 65.1% | 24.1% | 15.7% | 83.7% | 12.1% | 67.7% | 20.6% | 11.8% | 84.6% |
| 386 | 14.5% | 62.5% | 42.2% | 11.4% | 74.5% | 9.4% | 65.4% | 48.9% | 29.7% | 84.9% | 12.4% | 56.3% | 39.5% | 20.0% | 84.9% |
| 387 | 11.7% | 66.1% | 28.7% | 43.4% | 84.5% | 5.1% | 62.8% | 32.3% | 34.4% | 91.5% | 8.0% | 53.4% | 21.0% | 25.8% | 90.6% |
| 388 | 13.1% | 64.6% | 31.3% | 14.4% | 77.7% | 5.0% | 58.0% | 37.7% | 20.7% | 88.9% | 11.9% | 44.0% | 22.0% | 14.2% | 86.8% |
| 389 | 33.5% | 63.3% | 39.7% | 25.0% | 82.1% | 42.6% | 50.5% | 29.6% | 17.8% | 89.5% | 30.0% | 34.7% | 17.7% | 12.0% | 86.7% |
| 390 | 28.8% | 64.0% | 14.6% | 14.9% | 80.2% | 14.1% | 80.3% | 42.6% | 39.1% | 90.4% | 13.8% | 76.1% | 25.3% | 19.5% | 88.6% |
| 391 | 13.6% | 62.9% | 51.0% | 22.2% | 75.6% | 9.4% | 64.2% | 51.5% | 22.3% | 87.6% | 13.0% | 53.0% | 34.3% | 14.6% | 84.9% |
| 392 | 12.4% | 66.0% | 39.0% | 44.8% | 77.7% | 5.1% | 71.2% | 34.6% | 28.6% | 87.9% | 10.4% | 56.7% | 23.3% | 24.0% | 86.5% |
| 393 | 42.6% | 73.8% | 19.6% | 13.3% | 88.9% | 12.6% | 73.8% | 53.1% | 16.6% | 94.0% | 17.8% | 66.2% | 38.7% | 12.4% | 93.7% |
| 394 | 53.2% | 57.5% | 19.1% | 18.9% | 82.0% | 66.9% | 65.5% | 42.6% | 19.3% | 89.5% | 61.6% | 51.8% | 27.5% | 14.4% | 89.5% |
| 395 | 27.0% | 69.7% | 17.6% | 16.1% | 77.8% | 31.9% | 76.9% | 35.9% | 22.4% | 89.0% | 22.9% | 76.7% | 26.3% | 14.7% | 89.1% |
| 396 | 22.7% | 60.1% | 28.5% | 9.7% | 73.9% | 8.3% | 56.1% | 45.9% | 19.8% | 83.7% | 14.4% | 48.3% | 35.6% | 12.4% | 82.4% |
| 397 | 14.6% | 62.9% | 23.8% | 37.1% | 77.5% | 6.7% | 70.3% | 43.9% | 36.0% | 87.5% | 10.5% | 57.5% | 26.3% | 26.6% | 84.8% |
| 398 | 22.5% | 60.2% | 24.6% | 23.0% | 76.3% | 9.5% | 70.4% | 49.1% | 19.2% | 87.1% | 13.3% | 70.2% | 37.7% | 16.5% | 88.6% |
| 399 | 45.3% | 57.7% | 21.4% | 16.2% | 69.0% | 33.0% | 61.8% | 38.6% | 23.0% | 85.1% | 34.9% | 59.6% | 33.6% | 19.4% | 84.9% |
| 400 | 31.1% | 61.2% | 20.2% | 18.6% | 69.4% | 10.4% | 65.7% | 38.8% | 30.5% | 82.7% | 18.7% | 66.7% | 28.9% | 18.5% | 84.1% |
| 401 | 28.8% | 47.7% | 27.4% | 12.8% | 70.7% | 10.9% | 62.9% | 49.6% | 21.6% | 85.9% | 15.9% | 49.2% | 30.7% | 14.2% | 83.1% |
| 402 | 20.9% | 56.2% | 21.4% | 30.9% | 72.9% | 6.9% | 73.8% | 35.6% | 25.2% | 88.3% | 13.2% | 65.2% | 25.0% | 20.4% | 86.2% |
| 403 | 37.8% | 46.8% | 18.9% | 12.2% | 65.3% | 25.3% | 59.4% | 38.3% | 21.0% | 84.1% | 26.6% | 50.5% | 26.3% | 15.5% | 80.0% |
| 404 | 37.3% | 63.7% | 24.9% | 18.1% | 70.3% | 10.6% | 68.1% | 39.9% | 27.2% | 83.4% | 18.4% | 68.5% | 31.0% | 16.2% | 84.3% |
| 405 | 35.7% | 48.4% | 25.8% | 11.1% | 70.0% | 10.8% | 68.3% | 54.9% | 30.1% | 85.6% | 19.0% | 48.1% | 31.3% | 14.1% | 80.8% |
| 406 | 32.1% | 59.1% | 19.6% | 20.1% | 72.4% | 5.7% | 72.6% | 38.0% | 24.3% | 87.9% | 16.6% | 59.6% | 23.3% | 17.5% | 84.5% |
| 407 | 35.1% | 56.4% | 20.4% | 13.0% | 72.5% | 8.6% | 69.6% | 56.2% | 25.1% | 88.0% | 16.6% | 58.2% | 34.2% | 14.7% | 85.1% |
| 408 | 51.8% | 64.0% | 28.2% | 15.1% | 70.8% | 42.9% | 64.2% | 37.2% | 16.8% | 83.2% | 46.2% | 58.8% | 28.3% | 14.2% | 82.0% |
| 409 | 37.4% | 61.8% | 25.7% | 14.2% | 69.4% | 44.0% | 66.9% | 32.9% | 18.4% | 83.7% | 42.9% | 69.2% | 28.9% | 15.2% | 84.6% |
| 410 | 29.4% | 57.5% | 31.4% | 14.8% | 70.3% | 7.5% | 56.3% | 38.0% | 19.2% | 84.8% | 17.9% | 56.0% | 34.6% | 15.5% | 86.9% |
| 411 | 33.1% | 69.1% | 26.8% | 15.9% | 75.6% | 7.6% | 72.4% | 58.7% | 21.8% | 86.9% | 17.7% | 70.2% | 45.4% | 15.6% | 86.2% |
| 412 | 41.4% | 58.4% | 32.7% | 17.7% | 66.4% | 23.9% | 58.5% | 45.0% | 22.8% | 83.1% | 39.0% | 61.3% | 36.4% | 18.6% | 83.8% |
| 413 | 35.3% | 57.0% | 25.8% | 16.5% | 64.1% | 6.7% | 63.1% | 34.6% | 25.2% | 80.6% | 18.7% | 60.2% | 23.3% | 14.4% | 76.3% |
| 414 | 31.1% | 58.4% | 35.5% | 12.5% | 67.1% | 6.6% | 63.0% | 50.1% | 23.8% | 82.8% | 18.8% | 59.3% | 39.4% | 16.0% | 82.7% |
| 415 | 23.6% | 57.9% | 26.2% | 19.0% | 65.7% | 6.5% | 62.8% | 34.8% | 22.0% | 83.3% | 17.1% | 62.1% | 27.4% | 20.5% | 82.4% |
| 416 | 32.0% | 73.3% | 34.4% | 13.6% | 79.2% | 5.5% | 64.6% | 48.1% | 26.3% | 86.5% | 17.0% | 61.2% | 35.3% | 18.2% | 87.1% |
| 417 | 40.1% | 56.8% | 31.6% | 13.2% | 65.3% | 33.8% | 63.5% | 46.3% | 26.5% | 81.6% | 34.1% | 61.0% | 34.4% | 17.2% | 80.5% |
| 418 | 35.3% | 62.1% | 44.1% | 23.4% | 68.6% | 10.6% | 64.7% | 45.4% | 37.0% | 83.7% | 26.0% | 65.0% | 37.2% | 22.6% | 82.4% |
| 419 | 27.6% | 57.7% | 39.1% | 16.1% | 65.9% | 8.2% | 60.5% | 43.6% | 27.3% | 83.4% | 20.2% | 59.6% | 37.2% | 16.8% | 81.2% |
| 420 | 24.3% | 59.6% | 26.6% | 17.8% | 68.4% | 6.2% | 65.6% | 42.0% | 30.8% | 82.9% | 15.7% | 62.3% | 32.3% | 24.2% | 81.6% |
| 421 | 31.2% | 62.5% | 36.8% | 14.4% | 69.8% | 8.3% | 66.3% | 54.2% | 30.4% | 84.8% | 20.8% | 65.7% | 42.4% | 20.6% | 84.5% |
| 422 | 44.4% | 61.6% | 44.5% | 19.8% | 69.1% | 17.0% | 49.4% | 32.3% | 25.8% | 81.3% | 34.1% | 55.8% | 33.1% | 21.3% | 82.1% |
| 423 | 39.0% | 63.6% | 48.3% | 32.1% | 70.1% | 11.1% | 65.3% | 47.8% | 44.2% | 83.7% | 22.2% | 68.3% | 46.9% | 37.4% | 84.4% |
| 424 | 28.5% | 57.9% | 44.3% | 19.9% | 68.0% | 5.6% | 49.5% | 36.9% | 22.4% | 81.6% | 18.6% | 54.8% | 37.0% | 18.1% | 80.8% |
| 425 | 21.9% | 61.7% | 32.9% | 23.4% | 70.0% | 5.6% | 64.4% | 41.4% | 29.8% | 82.6% | 15.3% | 67.3% | 35.0% | 25.6% | 84.3% |
| 426 | 23.3% | 58.6% | 39.1% | 16.3% | 66.5% | 15.7% | 61.7% | 44.4% | 28.6% | 82.4% | 23.2% | 65.0% | 43.2% | 20.9% | 83.1% |
| 427 | 45.4% | 62.8% | 45.8% | 22.2% | 70.5% | 34.4% | 64.0% | 48.2% | 37.5% | 86.1% | 44.1% | 68.9% | 48.3% | 29.9% | 85.7% |
| 428 | 38.3% | 62.1% | 45.7% | 36.7% | 68.2% | 8.0% | 67.3% | 48.1% | 45.5% | 84.0% | 25.3% | 64.5% | 40.3% | 31.1% | 81.6% |
| 429 | 24.5% | 54.1% | 40.5% | 15.9% | 62.1% | 7.7% | 63.4% | 50.0% | 39.2% | 83.0% | 20.7% | 61.7% | 42.5% | 20.7% | 81.3% |
| 430 | 22.2% | 58.9% | 35.2% | 26.2% | 66.6% | 6.9% | 62.8% | 40.0% | 30.5% | 83.4% | 20.3% | 64.0% | 34.6% | 26.3% | 82.7% |
| 431 | 27.3% | 59.6% | 42.4% | 25.5% | 67.1% | 11.4% | 68.6% | 54.6% | 31.0% | 84.9% | 23.9% | 67.5% | 46.2% | 24.9% | 84.0% |
| 432 | 41.2% | 57.1% | 41.9% | 27.9% | 64.2% | 17.0% | 61.4% | 44.5% | 33.9% | 83.2% | 35.9% | 61.7% | 41.1% | 24.7% | 80.2% |
| 433 | 37.4% | 64.1% | 48.2% | 35.9% | 69.8% | 15.1% | 76.4% | 56.5% | 55.2% | 88.7% | 28.2% | 72.2% | 47.4% | 37.1% | 87.2% |
| 434 | 28.5% | 61.3% | 46.4% | 30.3% | 69.6% | 10.0% | 56.7% | 41.6% | 41.3% | 83.9% | 21.0% | 61.8% | 40.6% | 34.2% | 83.3% |
| 435 | 23.7% | 60.3% | 40.1% | 31.7% | 67.9% | 5.6% | 66.9% | 43.6% | 36.7% | 86.8% | 19.3% | 64.2% | 36.7% | 29.2% | 83.8% |
| 436 | 31.2% | 62.8% | 48.0% | 29.8% | 70.4% | 4.7% | 56.9% | 36.2% | 28.9% | 84.1% | 21.5% | 57.1% | 32.0% | 21.4% | 82.7% |
| 437 | 44.6% | 60.5% | 45.0% | 31.5% | 66.7% | 25.2% | 66.0% | 49.0% | 36.8% | 85.9% | 38.9% | 63.5% | 38.3% | 22.5% | 82.7% |
| 438 | 40.4% | 63.4% | 47.4% | 41.9% | 69.3% | 5.7% | 73.3% | 42.9% | 35.7% | 87.8% | 23.6% | 65.6% | 29.7% | 20.2% | 82.6% |
| 439 | 35.9% | 62.2% | 49.1% | 30.0% | 69.1% | 5.9% | 58.7% | 48.9% | 33.0% | 85.4% | 21.2% | 57.4% | 32.2% | 16.5% | 79.8% |
| 440 | 29.1% | 59.0% | 39.9% | 35.1% | 65.6% | 4.5% | 68.8% | 45.0% | 40.8% | 85.5% | 20.7% | 64.8% | 32.6% | 28.6% | 82.0% |
| 441 | 36.0% | 63.2% | 45.5% | 31.2% | 70.3% | 5.7% | 68.9% | 55.9% | 34.1% | 86.9% | 28.0% | 66.8% | 41.7% | 21.2% | 83.8% |
| 442 | 40.6% | 58.1% | 40.9% | 27.3% | 65.4% | 38.8% | 67.8% | 50.3% | 36.2% | 87.0% | 37.6% | 65.5% | 42.5% | 29.4% | 84.6% |
| 443 | 36.3% | 58.3% | 43.8% | 37.1% | 64.4% | 25.4% | 66.5% | 38.4% | 36.3% | 83.9% | 27.3% | 62.4% | 36.3% | 29.2% | 78.8% |
| 444 | 31.6% | 62.0% | 47.0% | 28.6% | 69.0% | 8.2% | 66.3% | 53.0% | 39.5% | 85.7% | 23.0% | 65.7% | 41.9% | 20.8% | 83.9% |
| 445 | 27.0% | 57.1% | 38.9% | 34.1% | 64.3% | 5.7% | 43.6% | 24.3% | 25.6% | 80.4% | 14.4% | 49.5% | 22.8% | 20.4% | 76.1% |
| 446 | 26.2% | 58.9% | 41.8% | 29.0% | 66.0% | 7.2% | 63.9% | 43.4% | 33.6% | 81.5% | 17.3% | 63.6% | 41.4% | 27.5% | 79.4% |
| 447 | 39.5% | 60.3% | 42.3% | 27.0% | 67.1% | 23.2% | 63.9% | 44.5% | 35.9% | 85.4% | 35.0% | 65.5% | 38.1% | 23.4% | 82.8% |
| 448 | 36.2% | 62.8% | 48.5% | 41.8% | 68.7% | 5.9% | 76.7% | 53.8% | 54.6% | 88.5% | 19.2% | 69.0% | 41.6% | 32.5% | 83.7% |
| 449 | 24.7% | 56.5% | 42.7% | 22.6% | 64.8% | 6.8% | 58.5% | 47.7% | 31.3% | 85.1% | 17.1% | 59.3% | 36.6% | 18.2% | 80.1% |
| 450 | 22.1% | 59.6% | 39.6% | 34.1% | 66.8% | 5.8% | 73.4% | 45.3% | 38.3% | 88.4% | 14.2% | 67.5% | 35.1% | 28.0% | 84.7% |
| 451 | 21.5% | 54.5% | 36.5% | 22.6% | 64.2% | 7.5% | 67.5% | 52.5% | 35.1% | 84.7% | 14.2% | 57.6% | 35.0% | 22.8% | 78.9% |
| 452 | 44.3% | 62.3% | 47.0% | 27.2% | 68.2% | 27.0% | 53.8% | 37.8% | 36.7% | 87.6% | 39.2% | 67.4% | 44.5% | 24.5% | 84.6% |
| 453 | 36.4% | 66.0% | 51.2% | 41.9% | 72.3% | 7.4% | 73.5% | 37.8% | 32.9% | 87.7% | 18.6% | 69.7% | 36.1% | 25.3% | 85.3% |
| 454 | 23.7% | 61.6% | 46.3% | 24.3% | 68.4% | 10.8% | 69.1% | 54.0% | 43.2% | 86.8% | 17.6% | 66.5% | 44.5% | 23.8% | 84.5% |
| 455 | 24.6% | 70.8% | 48.7% | 42.3% | 76.0% | 3.9% | 65.5% | 40.5% | 41.3% | 89.9% | 11.6% | 69.4% | 33.8% | 32.3% | 88.2% |
| 456 | 21.5% | 57.2% | 39.8% | 22.8% | 64.8% | 5.4% | 55.2% | 35.1% | 21.6% | 81.8% | 13.2% | 59.9% | 33.0% | 17.1% | 79.2% |
| 457 | 44.5% | 65.9% | 49.4% | 29.1% | 72.2% | 20.7% | 51.9% | 31.9% | 27.1% | 89.6% | 35.6% | 66.2% | 38.6% | 22.7% | 87.6% |
| 458 | 27.4% | 63.2% | 48.5% | 37.7% | 69.6% | 8.9% | 76.1% | 49.8% | 49.2% | 88.2% | 15.6% | 73.1% | 42.5% | 34.0% | 86.5% |
| 459 | 22.1% | 63.3% | 50.9% | 27.2% | 70.1% | 13.4% | 76.1% | 65.5% | 55.7% | 91.7% | 18.3% | 68.5% | 47.2% | 29.5% | 84.9% |
| 460 | 21.3% | 58.7% | 36.7% | 29.9% | 66.2% | 6.7% | 61.7% | 41.4% | 38.4% | 85.1% | 15.0% | 60.6% | 30.9% | 25.4% | 80.0% |
| 461 | 22.0% | 62.7% | 44.5% | 25.6% | 70.0% | 7.6% | 72.4% | 53.7% | 35.6% | 87.8% | 16.4% | 70.8% | 45.3% | 24.9% | 88.3% |
| 462 | 39.6% | 62.0% | 43.2% | 23.5% | 68.1% | 32.0% | 62.8% | 39.8% | 32.7% | 86.6% | 37.4% | 67.8% | 42.9% | 25.9% | 84.6% |
| 463 | 25.9% | 58.1% | 42.6% | 31.8% | 65.3% | 19.0% | 70.5% | 39.1% | 38.7% | 84.3% | 23.5% | 71.0% | 36.8% | 32.0% | 84.1% |
| 464 | 20.9% | 59.2% | 46.8% | 25.4% | 66.9% | 5.8% | 61.9% | 51.2% | 34.8% | 86.5% | 14.4% | 65.1% | 46.4% | 21.8% | 84.0% |
| 465 | 15.2% | 61.3% | 39.0% | 31.5% | 68.4% | 6.1% | 68.9% | 36.8% | 36.7% | 86.6% | 12.2% | 71.1% | 35.4% | 32.2% | 85.3% |
| 466 | 19.0% | 62.6% | 43.9% | 23.1% | 69.0% | 5.7% | 73.4% | 51.4% | 37.6% | 88.3% | 12.8% | 72.6% | 46.3% | 27.4% | 86.5% |
| 467 | 42.4% | 61.2% | 42.4% | 25.5% | 68.3% | 24.4% | 61.6% | 47.6% | 45.7% | 88.6% | 38.5% | 65.2% | 44.7% | 29.6% | 86.5% |
| 468 | 26.6% | 64.6% | 50.2% | 39.1% | 70.8% | 11.4% | 75.1% | 44.7% | 44.3% | 87.7% | 16.9% | 71.0% | 40.3% | 32.4% | 84.1% |
| 469 | 22.2% | 60.5% | 44.0% | 21.9% | 68.3% | 5.8% | 43.2% | 33.2% | 21.2% | 83.3% | 15.3% | 56.3% | 34.0% | 16.3% | 83.9% |
| 470 | 17.5% | 61.0% | 40.2% | 33.2% | 68.4% | 4.7% | 71.3% | 44.1% | 43.0% | 89.3% | 11.3% | 67.7% | 36.7% | 36.1% | 86.9% |
| 471 | 22.6% | 63.9% | 48.4% | 28.6% | 70.1% | 4.8% | 72.6% | 55.2% | 37.9% | 88.1% | 14.4% | 68.9% | 46.4% | 26.9% | 86.9% |
| 472 | 44.7% | 60.6% | 41.8% | 23.1% | 67.2% | 46.7% | 69.3% | 50.3% | 36.2% | 88.9% | 52.4% | 67.4% | 44.9% | 31.1% | 86.9% |
| 473 | 35.4% | 63.9% | 42.0% | 32.6% | 70.8% | 12.1% | 74.0% | 45.0% | 39.2% | 88.0% | 23.8% | 74.5% | 34.5% | 25.8% | 87.4% |
| 474 | 24.6% | 58.0% | 42.3% | 21.0% | 66.3% | 7.0% | 58.3% | 48.0% | 31.6% | 85.0% | 15.6% | 54.7% | 40.8% | 22.3% | 83.6% |
| 475 | 20.4% | 58.0% | 36.7% | 32.2% | 65.3% | 5.3% | 60.3% | 40.8% | 40.7% | 86.9% | 14.4% | 61.6% | 36.8% | 38.4% | 85.7% |
| 476 | 23.0% | 61.2% | 41.7% | 20.6% | 67.2% | 9.7% | 75.4% | 56.3% | 35.8% | 89.4% | 18.2% | 73.5% | 47.9% | 27.6% | 87.9% |
| 477 | 46.9% | 62.0% | 38.9% | 22.8% | 68.5% | 14.5% | 38.3% | 23.9% | 22.7% | 84.5% | 31.3% | 40.7% | 23.0% | 18.0% | 81.7% |
| 478 | 39.1% | 64.0% | 36.6% | 30.1% | 69.1% | 4.7% | 73.8% | 34.4% | 26.3% | 86.8% | 17.4% | 74.2% | 23.9% | 16.7% | 86.2% |
| 479 | 24.9% | 60.0% | 43.4% | 18.6% | 67.9% | 9.0% | 74.2% | 56.5% | 43.3% | 89.3% | 17.1% | 69.4% | 48.6% | 28.8% | 87.3% |
| 480 | 22.8% | 66.0% | 35.9% | 32.4% | 72.1% | 4.4% | 80.7% | 51.2% | 45.9% | 92.0% | 14.6% | 75.6% | 41.6% | 40.9% | 90.3% |
| 481 | 26.8% | 61.4% | 38.9% | 21.9% | 67.7% | 6.5% | 74.1% | 58.4% | 38.3% | 89.0% | 19.3% | 69.8% | 48.0% | 30.3% | 88.7% |
| 482 | 53.1% | 66.9% | 39.0% | 20.4% | 73.5% | 60.9% | 64.5% | 48.3% | 27.2% | 89.7% | 63.4% | 67.5% | 45.2% | 26.5% | 90.2% |
| 483 | 46.4% | 74.1% | 47.6% | 37.6% | 78.9% | 11.7% | 86.8% | 56.2% | 56.5% | 93.5% | 21.3% | 83.7% | 41.2% | 37.7% | 91.6% |
| 484 | 31.2% | 62.4% | 42.4% | 15.2% | 69.1% | 5.8% | 78.0% | 64.5% | 45.7% | 91.8% | 17.9% | 71.2% | 51.3% | 26.7% | 89.3% |
| 485 | 25.2% | 67.5% | 34.5% | 33.3% | 74.1% | 4.5% | 74.2% | 46.7% | 44.0% | 90.0% | 16.2% | 72.1% | 39.9% | 40.7% | 89.8% |
| 486 | 43.1% | 80.6% | 45.8% | 22.8% | 84.9% | 5.5% | 66.6% | 47.2% | 24.9% | 87.8% | 19.1% | 63.6% | 38.6% | 21.4% | 88.7% |
| 487 | 50.6% | 65.2% | 37.2% | 22.6% | 71.5% | 25.9% | 67.5% | 48.7% | 53.8% | 91.1% | 47.3% | 70.9% | 47.7% | 41.1% | 88.8% |
| 488 | 46.1% | 67.8% | 35.6% | 28.2% | 73.1% | 7.0% | 76.4% | 48.8% | 42.2% | 89.4% | 28.1% | 73.0% | 35.0% | 23.6% | 86.1% |
| 489 | 34.4% | 66.6% | 45.9% | 14.9% | 73.9% | 18.2% | 78.8% | 68.8% | 45.1% | 92.3% | 22.8% | 71.8% | 57.7% | 26.1% | 88.0% |
| 490 | 27.4% | 65.8% | 30.8% | 27.9% | 73.4% | 6.9% | 61.8% | 41.0% | 41.2% | 88.8% | 15.3% | 54.4% | 28.8% | 28.8% | 82.1% |
| 491 | 32.7% | 65.6% | 37.1% | 20.5% | 71.8% | 4.6% | 77.9% | 61.7% | 37.2% | 90.2% | 19.4% | 77.4% | 52.4% | 27.6% | 91.2% |
| 492 | 48.5% | 63.3% | 35.8% | 20.7% | 69.4% | 24.1% | 61.5% | 48.3% | 36.3% | 88.4% | 46.7% | 63.6% | 40.5% | 24.0% | 86.5% |
| 493 | 45.6% | 66.6% | 38.7% | 31.8% | 72.2% | 13.4% | 79.1% | 56.9% | 56.6% | 90.1% | 29.3% | 79.0% | 48.7% | 41.8% | 89.5% |
| 494 | 43.3% | 76.1% | 54.5% | 16.1% | 81.2% | 6.9% | 68.0% | 62.4% | 30.1% | 92.6% | 22.6% | 62.0% | 55.6% | 18.4% | 92.0% |
| 495 | 28.8% | 70.9% | 34.0% | 33.4% | 76.8% | 4.6% | 73.7% | 46.8% | 38.7% | 87.8% | 20.2% | 73.9% | 40.5% | 34.5% | 88.3% |
| 496 | 39.3% | 71.1% | 36.6% | 20.2% | 76.1% | 5.4% | 72.7% | 59.2% | 32.1% | 89.2% | 23.9% | 72.0% | 51.8% | 25.1% | 89.6% |
| 497 | 43.7% | 59.9% | 30.0% | 19.6% | 69.4% | 36.2% | 65.2% | 47.9% | 36.0% | 89.3% | 48.1% | 61.9% | 36.4% | 26.4% | 87.3% |
| 498 | 34.1% | 61.4% | 30.2% | 26.1% | 68.2% | 8.0% | 73.5% | 44.6% | 37.2% | 88.4% | 31.2% | 73.3% | 34.1% | 23.8% | 86.8% |
| 499 | 24.7% | 58.5% | 34.5% | 15.6% | 69.4% | 13.7% | 72.0% | 61.7% | 46.9% | 89.5% | 21.8% | 63.8% | 45.6% | 26.1% | 87.3% |
| 500 | 21.1% | 61.4% | 30.9% | 30.8% | 69.7% | 5.8% | 72.3% | 52.7% | 50.5% | 90.7% | 15.9% | 67.9% | 41.4% | 40.7% | 89.0% |
| 501 | 26.1% | 60.1% | 31.4% | 20.3% | 69.7% | 6.0% | 71.5% | 54.4% | 31.6% | 88.6% | 21.2% | 66.5% | 40.0% | 23.9% | 88.5% |
| 502 | 52.1% | 64.2% | 20.3% | 8.5% | 77.4% | 14.9% | 29.9% | 22.3% | 20.5% | 87.9% | 26.0% | 35.9% | 20.6% | 14.4% | 87.0% |
| 503 | 21.0% | 71.2% | 25.6% | 10.5% | 77.0% | 5.8% | 70.7% | 21.2% | 14.8% | 86.9% | 13.2% | 69.7% | 20.9% | 13.5% | 88.5% |
| 504 | 15.8% | 54.1% | 33.6% | 8.6% | 79.6% | 4.0% | 29.3% | 24.1% | 13.7% | 85.9% | 12.4% | 36.2% | 26.3% | 12.9% | 88.4% |
| 505 | 9.1% | 80.1% | 11.3% | 8.0% | 87.9% | 2.9% | 30.9% | 18.9% | 28.4% | 91.7% | 7.4% | 33.6% | 13.7% | 21.4% | 93.1% |
| 506 | 17.6% | 64.5% | 21.9% | 9.0% | 76.1% | 5.0% | 45.6% | 34.5% | 28.3% | 87.9% | 12.9% | 44.6% | 27.2% | 18.3% | 86.7% |
| 507 | 36.5% | 45.4% | 19.1% | 11.3% | 71.1% | 23.1% | 40.8% | 25.5% | 20.0% | 83.9% | 26.7% | 40.9% | 22.1% | 15.3% | 84.3% |
| 508 | 23.4% | 67.0% | 21.6% | 12.5% | 72.9% | 7.4% | 61.3% | 22.7% | 15.7% | 82.2% | 14.5% | 63.9% | 22.3% | 12.9% | 84.2% |
| 509 | 18.9% | 63.4% | 36.7% | 8.6% | 78.9% | 9.9% | 64.4% | 63.5% | 42.3% | 88.1% | 12.2% | 53.0% | 50.1% | 27.5% | 88.8% |
| 510 | 11.0% | 74.4% | 16.5% | 11.5% | 81.6% | 4.4% | 44.1% | 28.9% | 32.5% | 87.8% | 9.5% | 44.2% | 21.2% | 23.2% | 88.0% |
| 511 | 15.1% | 65.4% | 29.1% | 11.4% | 74.7% | 5.0% | 55.2% | 35.4% | 24.5% | 86.6% | 13.6% | 47.7% | 24.9% | 16.5% | 86.1% |
| 512 | 55.4% | 70.2% | 29.3% | 19.3% | 85.0% | 32.7% | 48.2% | 34.4% | 30.2% | 89.9% | 38.9% | 42.5% | 24.0% | 21.0% | 91.0% |
| 513 | 26.8% | 69.0% | 24.2% | 22.6% | 74.6% | 9.1% | 66.9% | 32.3% | 22.2% | 84.7% | 15.8% | 67.9% | 23.4% | 14.8% | 85.2% |
| 514 | 19.3% | 61.6% | 39.3% | 12.7% | 74.3% | 7.0% | 51.2% | 37.1% | 20.4% | 83.7% | 15.4% | 48.9% | 30.0% | 15.2% | 86.2% |
| 515 | 12.7% | 62.8% | 29.0% | 32.2% | 74.0% | 4.7% | 53.9% | 24.8% | 24.2% | 85.2% | 12.0% | 44.2% | 18.8% | 17.9% | 84.8% |
| 516 | 15.9% | 66.9% | 34.3% | 17.7% | 76.0% | 5.5% | 62.9% | 40.6% | 28.1% | 88.5% | 12.1% | 49.7% | 26.2% | 18.1% | 86.6% |
| 517 | 34.8% | 50.9% | 27.0% | 27.9% | 73.1% | 34.2% | 56.3% | 37.1% | 26.6% | 86.7% | 32.1% | 49.4% | 26.5% | 19.6% | 87.4% |
| 518 | 24.1% | 68.2% | 23.7% | 22.9% | 74.3% | 7.4% | 67.3% | 30.9% | 19.4% | 85.7% | 15.4% | 69.0% | 24.8% | 15.6% | 86.9% |
| 519 | 18.3% | 66.8% | 48.0% | 20.1% | 81.1% | 8.0% | 62.2% | 52.8% | 21.1% | 87.4% | 13.8% | 48.9% | 36.6% | 13.7% | 86.5% |
| 520 | 14.4% | 58.4% | 28.0% | 37.0% | 72.7% | 4.9% | 54.0% | 32.6% | 29.3% | 84.8% | 12.4% | 45.9% | 23.1% | 22.1% | 83.4% |
| 521 | 15.3% | 62.1% | 36.6% | 23.3% | 77.7% | 9.0% | 60.3% | 39.1% | 23.6% | 88.0% | 12.8% | 46.5% | 24.8% | 16.8% | 86.0% |
| 522 | 40.1% | 58.6% | 26.0% | 27.4% | 79.8% | 39.3% | 55.4% | 35.7% | 30.5% | 87.3% | 33.0% | 47.9% | 27.2% | 21.4% | 87.7% |
| 523 | 26.4% | 63.4% | 22.5% | 21.8% | 70.0% | 18.6% | 68.4% | 32.2% | 23.9% | 85.3% | 21.5% | 70.6% | 26.7% | 17.5% | 86.6% |
| 524 | 19.0% | 52.0% | 33.9% | 12.8% | 76.0% | 24.1% | 59.1% | 55.6% | 29.2% | 87.0% | 16.7% | 44.9% | 37.0% | 15.2% | 84.0% |
| 525 | 20.7% | 60.8% | 21.8% | 32.1% | 76.6% | 6.4% | 66.1% | 37.1% | 35.9% | 88.3% | 13.3% | 54.5% | 25.1% | 25.7% | 87.2% |
| 526 | 21.3% | 54.5% | 22.9% | 21.2% | 77.6% | 13.3% | 66.8% | 52.0% | 34.9% | 89.3% | 14.1% | 55.2% | 36.9% | 24.1% | 88.9% |
| 527 | 45.3% | 57.5% | 24.2% | 19.0% | 71.7% | 33.2% | 62.1% | 40.3% | 28.7% | 87.4% | 35.7% | 55.5% | 29.0% | 19.9% | 86.1% |
| 528 | 33.6% | 49.0% | 27.7% | 12.7% | 73.3% | 20.5% | 62.5% | 54.5% | 31.4% | 85.4% | 20.1% | 54.1% | 39.1% | 19.2% | 84.4% |
| 529 | 21.1% | 56.9% | 24.9% | 32.2% | 72.6% | 12.2% | 70.3% | 34.7% | 22.1% | 86.9% | 15.8% | 63.9% | 26.9% | 20.3% | 85.9% |
| 530 | 27.1% | 60.9% | 22.2% | 16.7% | 73.3% | 12.6% | 69.5% | 51.4% | 23.9% | 87.8% | 17.1% | 64.3% | 41.0% | 17.5% | 87.2% |
| 531 | 38.6% | 60.8% | 23.5% | 17.7% | 68.0% | 13.8% | 65.7% | 37.7% | 28.6% | 84.8% | 23.5% | 67.4% | 27.7% | 17.1% | 85.1% |
| 532 | 31.5% | 49.0% | 24.7% | 11.7% | 69.0% | 29.3% | 51.0% | 34.1% | 18.0% | 82.6% | 26.6% | 49.0% | 29.3% | 16.6% | 82.9% |
| 533 | 23.3% | 57.7% | 19.8% | 21.1% | 70.9% | 9.4% | 70.2% | 41.4% | 29.5% | 86.7% | 17.5% | 58.2% | 24.3% | 20.2% | 85.3% |
| 534 | 31.4% | 61.8% | 24.5% | 15.6% | 72.6% | 6.5% | 74.5% | 56.8% | 25.2% | 89.8% | 19.6% | 60.1% | 33.7% | 16.2% | 87.8% |
| 535 | 36.5% | 55.2% | 27.0% | 14.9% | 62.7% | 36.2% | 58.7% | 43.6% | 23.1% | 81.1% | 37.4% | 57.6% | 34.0% | 17.1% | 81.3% |
| 536 | 38.4% | 64.3% | 25.9% | 17.8% | 71.2% | 13.2% | 68.8% | 44.5% | 33.7% | 84.3% | 26.9% | 69.5% | 27.8% | 16.9% | 85.0% |
| 537 | 27.2% | 62.1% | 33.6% | 13.0% | 75.3% | 19.5% | 67.1% | 46.1% | 17.0% | 87.2% | 20.5% | 53.3% | 33.2% | 13.3% | 86.3% |
| 538 | 26.0% | 59.0% | 23.3% | 18.4% | 67.9% | 9.0% | 61.5% | 30.3% | 24.0% | 81.1% | 20.3% | 61.3% | 25.4% | 19.5% | 82.7% |
| 539 | 33.4% | 70.6% | 26.0% | 14.7% | 78.7% | 15.2% | 76.8% | 64.5% | 19.2% | 89.3% | 22.7% | 74.3% | 46.8% | 14.2% | 89.9% |
| 540 | 47.6% | 64.2% | 33.7% | 16.0% | 71.4% | 34.9% | 65.7% | 44.7% | 22.4% | 85.4% | 39.6% | 67.0% | 35.1% | 20.0% | 84.5% |
| 541 | 43.5% | 64.0% | 37.6% | 24.3% | 70.6% | 7.2% | 67.1% | 42.3% | 32.5% | 84.7% | 21.6% | 69.0% | 32.4% | 20.1% | 84.4% |
| 542 | 29.1% | 58.0% | 37.1% | 13.6% | 66.6% | 7.8% | 60.0% | 47.6% | 27.1% | 82.5% | 20.2% | 59.5% | 38.9% | 17.1% | 81.6% |
| 543 | 24.5% | 60.9% | 30.2% | 23.2% | 69.0% | 7.8% | 62.3% | 34.3% | 26.3% | 84.2% | 16.8% | 63.1% | 30.5% | 24.2% | 84.5% |
| 544 | 29.2% | 59.7% | 32.0% | 16.2% | 67.7% | 7.5% | 60.1% | 44.1% | 25.6% | 84.2% | 21.6% | 62.9% | 37.6% | 19.9% | 86.6% |
| 545 | 44.2% | 61.7% | 39.2% | 15.1% | 69.8% | 29.1% | 63.0% | 48.0% | 30.6% | 85.2% | 38.4% | 65.3% | 38.6% | 20.3% | 85.5% |
| 546 | 36.8% | 59.3% | 43.6% | 25.0% | 65.9% | 7.2% | 63.1% | 42.5% | 33.2% | 83.1% | 23.8% | 65.7% | 37.7% | 24.7% | 82.8% |
| 547 | 27.9% | 58.2% | 40.7% | 16.1% | 66.1% | 8.2% | 60.2% | 45.2% | 29.8% | 82.8% | 21.9% | 63.3% | 40.2% | 20.5% | 84.4% |
| 548 | 20.9% | 58.2% | 28.4% | 19.1% | 66.4% | 5.9% | 63.8% | 39.5% | 31.2% | 84.1% | 16.0% | 63.9% | 29.7% | 23.8% | 82.9% |
| 549 | 43.1% | 66.6% | 50.2% | 26.2% | 71.9% | 16.5% | 65.4% | 47.7% | 39.5% | 84.2% | 29.6% | 68.5% | 44.0% | 27.7% | 84.5% |
| 550 | 27.7% | 59.0% | 44.3% | 17.5% | 66.4% | 6.4% | 60.4% | 47.6% | 32.0% | 84.2% | 24.7% | 61.6% | 41.0% | 19.0% | 82.3% |
| 551 | 20.4% | 57.5% | 30.3% | 20.1% | 65.0% | 7.3% | 55.8% | 38.7% | 32.9% | 79.6% | 16.6% | 58.9% | 34.5% | 27.1% | 79.1% |
| 552 | 27.7% | 59.1% | 40.4% | 19.5% | 66.8% | 9.2% | 63.1% | 47.2% | 34.0% | 83.7% | 22.9% | 66.2% | 44.6% | 24.8% | 83.5% |
| 553 | 42.1% | 58.7% | 43.5% | 29.4% | 66.4% | 21.2% | 58.4% | 44.0% | 35.3% | 84.0% | 39.1% | 63.6% | 43.7% | 29.1% | 82.6% |
| 554 | 42.7% | 65.8% | 49.2% | 37.4% | 71.0% | 7.0% | 68.5% | 49.3% | 46.7% | 85.3% | 27.6% | 69.1% | 43.3% | 32.9% | 84.3% |
| 555 | 27.6% | 57.7% | 40.3% | 17.3% | 65.5% | 6.1% | 60.2% | 46.2% | 32.6% | 84.9% | 21.6% | 62.6% | 41.9% | 22.2% | 86.5% |
| 556 | 23.2% | 57.8% | 38.8% | 27.8% | 64.7% | 6.7% | 62.2% | 43.3% | 39.0% | 82.7% | 20.2% | 62.1% | 39.2% | 31.7% | 81.8% |
| 557 | 28.8% | 62.4% | 42.7% | 24.3% | 69.4% | 6.2% | 65.1% | 47.6% | 30.3% | 85.7% | 20.0% | 68.9% | 44.2% | 24.3% | 87.5% |
| 558 | 42.2% | 60.8% | 43.8% | 29.3% | 68.3% | 19.6% | 54.4% | 38.4% | 35.7% | 84.3% | 34.5% | 63.6% | 38.7% | 24.6% | 84.0% |
| 559 | 35.1% | 61.3% | 43.6% | 27.4% | 68.3% | 6.3% | 60.3% | 47.3% | 31.2% | 83.8% | 27.0% | 60.6% | 39.4% | 21.3% | 82.4% |
| 560 | 29.9% | 58.1% | 39.2% | 34.4% | 66.4% | 6.3% | 63.1% | 43.4% | 41.6% | 85.5% | 26.5% | 62.4% | 33.7% | 28.0% | 85.1% |
| 561 | 33.5% | 64.8% | 49.1% | 33.7% | 71.0% | 7.7% | 64.2% | 45.1% | 37.4% | 83.6% | 25.7% | 66.6% | 42.9% | 29.4% | 84.9% |
| 562 | 49.6% | 65.5% | 49.5% | 36.4% | 71.8% | 28.5% | 56.9% | 43.1% | 48.1% | 88.0% | 42.3% | 67.1% | 45.2% | 32.2% | 85.3% |
| 563 | 29.4% | 60.9% | 43.3% | 28.8% | 68.5% | 11.5% | 61.8% | 50.5% | 49.8% | 87.3% | 23.9% | 64.0% | 39.8% | 27.4% | 83.6% |
| 564 | 28.6% | 58.2% | 40.9% | 34.9% | 65.1% | 6.3% | 70.5% | 50.0% | 45.4% | 87.1% | 27.9% | 62.6% | 35.0% | 29.5% | 82.0% |
| 565 | 31.4% | 63.3% | 44.4% | 30.2% | 69.7% | 7.1% | 72.2% | 55.8% | 39.1% | 88.7% | 27.5% | 68.0% | 42.2% | 25.1% | 86.8% |
| 566 | 50.0% | 71.8% | 59.3% | 52.7% | 76.7% | 6.3% | 71.9% | 36.8% | 31.6% | 87.0% | 30.7% | 71.0% | 30.3% | 22.4% | 84.6% |
| 567 | 33.2% | 59.6% | 46.5% | 28.6% | 65.3% | 7.0% | 58.7% | 48.2% | 36.0% | 83.9% | 27.0% | 58.8% | 34.2% | 18.5% | 79.0% |
| 568 | 29.0% | 60.8% | 41.1% | 36.0% | 68.5% | 3.9% | 49.7% | 35.1% | 35.4% | 84.5% | 21.8% | 62.6% | 30.8% | 26.2% | 83.9% |
| 569 | 30.8% | 60.9% | 44.2% | 28.5% | 68.4% | 7.1% | 65.5% | 48.4% | 37.3% | 87.9% | 22.9% | 65.1% | 40.2% | 24.7% | 85.1% |
| 570 | 40.6% | 61.1% | 43.2% | 25.2% | 67.3% | 19.8% | 55.9% | 36.4% | 35.0% | 85.0% | 34.1% | 64.2% | 38.3% | 21.9% | 84.7% |
| 571 | 27.9% | 60.9% | 45.1% | 37.2% | 68.0% | 9.9% | 68.9% | 34.9% | 42.3% | 85.3% | 19.4% | 69.3% | 38.8% | 33.3% | 85.8% |
| 572 | 25.1% | 57.7% | 42.4% | 23.0% | 65.0% | 6.3% | 57.1% | 47.7% | 38.5% | 84.3% | 17.0% | 62.5% | 37.6% | 21.3% | 83.5% |
| 573 | 21.3% | 57.9% | 38.4% | 32.2% | 65.8% | 6.6% | 57.1% | 39.1% | 40.0% | 88.4% | 16.7% | 63.1% | 36.0% | 30.4% | 85.8% |
| 574 | 35.6% | 72.5% | 49.3% | 33.0% | 77.5% | 5.6% | 82.2% | 70.8% | 48.5% | 92.1% | 20.2% | 79.7% | 57.3% | 31.5% | 91.1% |
| 575 | 48.9% | 65.7% | 46.6% | 28.0% | 71.6% | 25.9% | 58.3% | 41.5% | 39.0% | 88.0% | 43.0% | 68.8% | 39.1% | 25.0% | 86.8% |
| 576 | 35.4% | 67.7% | 53.4% | 43.3% | 73.7% | 6.5% | 50.9% | 22.3% | 17.3% | 81.1% | 14.9% | 62.3% | 25.8% | 17.0% | 84.1% |
| 577 | 21.6% | 57.9% | 42.6% | 22.1% | 65.4% | 7.0% | 57.5% | 47.3% | 32.9% | 88.2% | 15.7% | 63.3% | 38.3% | 20.2% | 84.3% |
| 578 | 21.2% | 62.6% | 42.3% | 36.1% | 69.2% | 6.4% | 51.7% | 36.0% | 43.2% | 85.2% | 14.8% | 62.1% | 32.4% | 29.9% | 83.8% |
| 579 | 39.3% | 60.2% | 42.3% | 24.1% | 66.7% | 16.2% | 43.0% | 28.2% | 24.8% | 84.6% | 31.2% | 64.1% | 36.4% | 20.3% | 84.8% |
| 580 | 30.9% | 62.5% | 47.9% | 38.7% | 69.0% | 6.3% | 66.2% | 31.2% | 29.3% | 83.5% | 16.8% | 66.7% | 36.5% | 25.9% | 83.9% |
| 581 | 20.1% | 58.0% | 44.3% | 23.4% | 65.2% | 7.8% | 47.4% | 39.6% | 24.7% | 83.2% | 16.5% | 61.2% | 38.3% | 17.2% | 80.2% |
| 582 | 20.7% | 61.3% | 39.8% | 32.0% | 68.1% | 4.6% | 61.4% | 40.5% | 38.7% | 86.7% | 15.1% | 66.4% | 34.5% | 29.1% | 84.2% |
| 583 | 22.0% | 59.6% | 41.7% | 22.8% | 65.9% | 5.9% | 55.7% | 36.2% | 26.0% | 82.5% | 16.5% | 64.6% | 36.2% | 21.9% | 84.7% |
| 584 | 43.3% | 62.0% | 44.2% | 27.8% | 68.8% | 21.1% | 53.6% | 40.5% | 34.9% | 87.3% | 36.3% | 65.0% | 42.0% | 25.8% | 88.2% |
| 585 | 32.6% | 63.8% | 48.8% | 40.4% | 70.0% | 6.7% | 72.4% | 35.8% | 31.3% | 87.6% | 17.1% | 72.2% | 33.4% | 23.2% | 85.5% |
| 586 | 21.9% | 60.1% | 46.1% | 25.8% | 66.5% | 5.9% | 47.6% | 39.5% | 26.4% | 83.9% | 15.3% | 63.5% | 40.0% | 16.5% | 83.7% |
| 587 | 18.5% | 61.5% | 40.8% | 34.0% | 67.0% | 4.3% | 45.7% | 32.3% | 38.2% | 84.3% | 12.7% | 62.1% | 31.0% | 29.9% | 86.0% |
| 588 | 19.9% | 63.0% | 47.2% | 28.6% | 69.9% | 4.8% | 69.8% | 53.7% | 41.0% | 88.6% | 14.4% | 71.0% | 48.2% | 29.2% | 86.9% |
| 589 | 41.3% | 59.6% | 43.9% | 29.2% | 66.5% | 16.6% | 45.0% | 32.6% | 37.8% | 89.7% | 38.0% | 64.0% | 40.6% | 29.4% | 89.2% |
| 590 | 31.8% | 62.1% | 48.1% | 39.0% | 67.6% | 6.5% | 72.5% | 35.9% | 33.3% | 86.5% | 17.3% | 73.3% | 33.7% | 25.6% | 85.7% |
| 591 | 19.2% | 54.9% | 35.5% | 28.8% | 61.4% | 4.5% | 54.7% | 35.6% | 38.0% | 83.2% | 14.3% | 58.9% | 30.4% | 30.0% | 82.1% |
| 592 | 22.6% | 63.3% | 45.6% | 28.2% | 69.4% | 4.7% | 61.8% | 44.8% | 35.3% | 88.3% | 13.7% | 64.4% | 40.3% | 27.6% | 87.5% |
| 593 | 64.2% | 77.0% | 57.8% | 32.5% | 81.2% | 26.1% | 59.0% | 46.8% | 38.6% | 94.0% | 58.6% | 63.7% | 42.9% | 32.0% | 93.8% |
| 594 | 42.4% | 73.8% | 57.3% | 46.0% | 78.2% | 6.5% | 80.4% | 27.7% | 24.0% | 91.0% | 17.4% | 83.5% | 25.6% | 19.2% | 91.8% |
| 595 | 22.7% | 60.6% | 42.8% | 25.5% | 68.4% | 10.1% | 64.6% | 47.4% | 44.1% | 88.1% | 20.1% | 65.1% | 44.3% | 32.0% | 89.2% |
| 596 | 23.8% | 59.8% | 34.5% | 28.8% | 67.8% | 5.6% | 59.7% | 41.1% | 42.9% | 87.6% | 17.0% | 60.1% | 35.7% | 39.6% | 87.8% |
| 597 | 26.9% | 63.7% | 40.2% | 23.5% | 70.3% | 5.7% | 70.0% | 53.9% | 40.7% | 90.2% | 16.7% | 63.9% | 40.1% | 31.1% | 90 |
| 598 | 49.0% | 63.0% | 37.6% | 24.0% | 69.4% | 16.5% | 50.1% | 36.6% | 33.8% | 88.5% | 45.5% | 53.7% | 34.4% | 27.4% | 86.3% |
| 599 | 33.8% | 63.2% | 45.4% | 37.0% | 69.2% | 10.4% | 78.3% | 41.1% | 57.5% | 89.5% | 19.8% | 79.5% | 41.1% | 50.7% | 89.5% |
| 600 | 25.9% | 59.3% | 42.9% | 20.5% | 66.7% | 9.9% | 64.9% | 52.9% | 46.6% | 86.6% | 19.4% | 65.2% | 48.0% | 35.0% | 86.2% |
| 601 | 23.7% | 65.5% | 35.8% | 32.2% | 71.2% | 4.8% | 58.5% | 54.4% | 55.3% | 90.6% | 15.7% | 62.4% | 46.6% | 49.8% | 89.6% |
| 602 | 29.7% | 64.2% | 38.5% | 24.2% | 71.1% | 6.8% | 72.3% | 57.7% | 43.2% | 89.1% | 18.1% | 68.0% | 46.5% | 33.2% | 89.9% |
| 603 | 46.2% | 61.7% | 38.8% | 22.7% | 68.2% | 23.6% | 51.9% | 50.4% | 51.2% | 89.6% | 43.2% | 67.3% | 48.8% | 39.4% | 88.9% |
| 604 | 47.1% | 71.6% | 44.2% | 34.6% | 76.2% | 6.7% | 68.6% | 28.6% | 23.1% | 85.6% | 21.3% | 75.7% | 26.4% | 17.9% | 88.8% |
| 605 | 29.9% | 68.9% | 36.4% | 34.5% | 74.2% | 3.5% | 61.9% | 44.7% | 51.1% | 92.7% | 18.9% | 64.4% | 32.9% | 41.2% | 92.1% |
| 606 | 40.5% | 79.2% | 43.0% | 20.6% | 83.6% | 5.4% | 78.3% | 55.5% | 37.0% | 93.6% | 25.6% | 76.4% | 44.9% | 30.7% | 93.6% |
| 607 | 49.7% | 65.8% | 37.8% | 20.5% | 72.3% | 18.2% | 58.4% | 33.1% | 30.8% | 89.0% | 45.7% | 63.9% | 35.9% | 24.2% | 87.7% |
| 608 | 43.9% | 65.1% | 36.2% | 30.5% | 70.3% | 6.5% | 78.8% | 55.8% | 49.6% | 88.7% | 29.2% | 75.7% | 38.8% | 29.6% | 87.9% |
| 609 | 30.7% | 61.8% | 42.2% | 16.1% | 69.2% | 8.3% | 68.0% | 53.1% | 40.3% | 89.4% | 23.7% | 62.3% | 43.5% | 26.3% | 86.0% |
| 610 | 29.5% | 66.5% | 34.1% | 31.6% | 73.5% | 5.8% | 67.3% | 48.4% | 48.5% | 90.3% | 23.0% | 66.2% | 38.5% | 37.1% | 87.9% |
| 611 | 33.6% | 65.7% | 35.7% | 20.6% | 72.4% | 4.8% | 75.1% | 58.2% | 37.0% | 91.5% | 22.4% | 69.7% | 44.6% | 27.2% | 90.5% |
| 612 | 48.8% | 64.4% | 36.8% | 20.8% | 70.9% | 22.5% | 51.8% | 35.6% | 25.3% | 85.1% | 47.2% | 65.1% | 43.2% | 25.6% | 86.4% |
| 613 | 46.1% | 66.3% | 41.9% | 37.1% | 72.0% | 8.6% | 67.6% | 36.2% | 36.4% | 85.0% | 22.8% | 73.3% | 40.1% | 37.0% | 85.9% |
| 614 | 32.3% | 61.3% | 41.2% | 16.8% | 67.9% | 6.6% | 59.3% | 47.0% | 30.7% | 87.4% | 22.0% | 58.7% | 42.1% | 21.7% | 87.9% |
| 615 | 25.3% | 61.3% | 32.9% | 28.6% | 68.4% | 6.2% | 63.1% | 43.2% | 41.1% | 86.7% | 25.3% | 65.2% | 38.3% | 34.7% | 87.7% |
| 616 | 31.2% | 62.1% | 33.4% | 20.1% | 68.8% | 5.6% | 71.8% | 55.8% | 39.5% | 88.4% | 25.5% | 68.5% | 45.8% | 29.2% | 88.0% |
| 617 | 43.1% | 60.3% | 31.5% | 21.1% | 69.7% | 31.2% | 66.7% | 47.1% | 30.7% | 88.2% | 44.8% | 69.0% | 40.7% | 25.3% | 88.5% |
| 618 | 34.3% | 63.5% | 34.5% | 28.3% | 70.5% | 6.8% | 72.2% | 31.6% | 29.8% | 87.2% | 23.9% | 72.6% | 26.4% | 20.3% | 88.4% |
| 619 | 24.9% | 58.0% | 34.5% | 16.7% | 67.6% | 10.3% | 66.4% | 52.5% | 32.0% | 88.1% | 21.6% | 60.6% | 39.9% | 19.1% | 85.8% |
| 620 | 21.2% | 58.1% | 28.7% | 29.3% | 66.7% | 6.8% | 69.8% | 47.4% | 37.7% | 87.8% | 20.6% | 67.6% | 41.6% | 33.7% | 85.8% |
| 621 | 26.8% | 62.7% | 33.1% | 20.9% | 71.3% | 5.4% | 73.0% | 55.0% | 31.1% | 90.3% | 23.0% | 67.9% | 39.9% | 21.0% | 89.4% |
| 622 | 46.4% | 66.4% | 22.7% | 9.9% | 76.6% | 25.5% | 38.4% | 27.2% | 30.9% | 84.9% | 32.9% | 43.6% | 25.7% | 21.3% | 87.3% |
| 623 | 21.8% | 72.0% | 21.1% | 9.9% | 78.1% | 6.5% | 54.9% | 18.7% | 12.6% | 85.4% | 14.5% | 60.4% | 20.4% | 13.3% | 87.0% |
| 624 | 16.0% | 67.3% | 42.8% | 6.9% | 79.3% | 4.1% | 26.9% | 21.1% | 13.6% | 85.1% | 12.6% | 34.4% | 22.2% | 12.2% | 85.8% |
| 625 | 9.8% | 83.6% | 9.9% | 5.3% | 88.5% | 3.3% | 43.8% | 17.5% | 27.0% | 92.5% | 8.0% | 48.3% | 14.7% | 19.3% | 93.0% |
| 626 | 15.8% | 63.7% | 24.4% | 8.8% | 78.1% | 4.2% | 43.8% | 25.7% | 18.5% | 87.1% | 11.9% | 42.6% | 22.5% | 14.2% | 86.2% |
| 627 | 41.5% | 54.0% | 21.6% | 12.4% | 72.2% | 19.8% | 48.1% | 33.1% | 23.1% | 85.3% | 28.4% | 47.7% | 27.7% | 17.3% | 86.6% |
| 628 | 21.0% | 68.4% | 25.6% | 14.5% | 76.1% | 5.7% | 55.7% | 19.1% | 14.9% | 83.6% | 13.7% | 56.1% | 20.2% | 13.9% | 86.6% |
| 629 | 14.7% | 71.8% | 35.8% | 8.2% | 81.6% | 4.7% | 27.3% | 19.7% | 12.3% | 86.4% | 11.4% | 34.1% | 18.8% | 11.3% | 88.0% |
| 630 | 14.4% | 65.7% | 19.6% | 13.4% | 73.9% | 5.8% | 36.1% | 18.8% | 21.3% | 83.4% | 13.5% | 45.9% | 22.2% | 18.5% | 85.5% |
| 631 | 14.4% | 64.4% | 26.0% | 10.3% | 74.8% | 6.2% | 39.6% | 21.5% | 15.4% | 85.0% | 10.8% | 39.1% | 19.3% | 13.0% | 85.0% |
| 632 | 35.9% | 60.9% | 30.3% | 25.7% | 72.5% | 34.4% | 58.2% | 51.0% | 22.6% | 86.2% | 37.7% | 55.4% | 42.2% | 18.7% | 85.5% |
| 633 | 25.2% | 66.8% | 29.7% | 21.0% | 72.6% | 7.0% | 66.1% | 31.7% | 20.5% | 84.3% | 15.7% | 69.3% | 25.6% | 15.6% | 85.2% |
| 634 | 15.0% | 65.7% | 49.8% | 19.3% | 75.8% | 18.0% | 64.5% | 54.3% | 21.5% | 86.6% | 16.4% | 59.6% | 45.9% | 16.8% | 88.0% |
| 635 | 8.5% | 79.7% | 21.7% | 19.9% | 84.9% | 4.1% | 32.9% | 17.4% | 34.6% | 89.4% | 10.0% | 39.7% | 16.1% | 19.6% | 88.2% |
| 636 | 25.9% | 70.5% | 27.1% | 17.1% | 82.4% | 6.0% | 51.6% | 26.1% | 18.7% | 87.3% | 12.5% | 50.9% | 23.5% | 15.4% | 89.8% |
| 637 | 41.3% | 48.0% | 23.1% | 18.6% | 71.2% | 22.4% | 47.5% | 27.0% | 20.0% | 86.3% | 34.0% | 46.9% | 22.6% | 16.3% | 86.6% |
| 638 | 27.4% | 65.8% | 26.6% | 27.0% | 73.8% | 16.4% | 67.7% | 36.9% | 25.4% | 87.4% | 20.4% | 70.5% | 31.1% | 19.3% | 88.7% |
| 639 | 20.3% | 67.2% | 48.2% | 18.7% | 78.7% | 8.4% | 38.8% | 35.1% | 23.5% | 88.2% | 15.2% | 42.9% | 28.0% | 15.1% | 88.3% |
| 640 | 22.3% | 58.4% | 21.6% | 30.7% | 77.7% | 6.0% | 56.8% | 22.8% | 38.1% | 91.7% | 15.5% | 48.0% | 17.5% | 27.5% | 91.0% |
| 641 | 20.6% | 65.3% | 26.8% | 24.1% | 84.2% | 10.9% | 69.3% | 45.9% | 24.4% | 90.7% | 14.1% | 56.1% | 30.4% | 17.9% | 90.2% |
| 642 | 54.1% | 64.3% | 22.2% | 21.9% | 79.4% | 29.3% | 51.5% | 27.7% | 22.0% | 89.7% | 48.9% | 49.5% | 21.8% | 14.9% | 90.1% |
| 643 | 30.0% | 69.2% | 22.2% | 22.2% | 75.3% | 10.6% | 69.6% | 29.6% | 19.7% | 87.9% | 19.0% | 73.9% | 25.7% | 15.1% | 87.8% |
| 644 | 26.6% | 65.3% | 33.3% | 10.2% | 83.5% | 8.5% | 54.6% | 38.5% | 16.7% | 91.0% | 17.2% | 45.9% | 27.0% | 12.3% | 91.0% |
| 645 | 16.0% | 54.6% | 22.0% | 34.6% | 76.2% | 5.8% | 40.7% | 21.4% | 32.9% | 86.8% | 15.2% | 42.0% | 19.5% | 22.6% | 88.4% |
| 646 | 23.9% | 65.2% | 20.2% | 19.3% | 83.8% | 10.1% | 78.2% | 51.7% | 17.1% | 91.7% | 14.9% | 65.7% | 29.9% | 13.0% | 91.5% |
| 647 | 45.2% | 61.1% | 27.6% | 24.4% | 72.5% | 38.9% | 63.7% | 39.8% | 24.7% | 88.5% | 44.9% | 64.4% | 33.4% | 20.1% | 88.8% |
| 648 | 33.7% | 67.1% | 21.8% | 19.0% | 73.9% | 32.7% | 73.4% | 20.6% | 18.3% | 87.6% | 32.4% | 75.6% | 21.5% | 14.4% | 89.6% |
| 649 | 27.5% | 52.5% | 28.3% | 12.9% | 72.0% | 11.5% | 60.4% | 47.0% | 22.8% | 86.3% | 19.9% | 53.2% | 34.9% | 16.5% | 86.3% |
| 650 | 23.0% | 58.4% | 21.6% | 27.0% | 73.2% | 10.1% | 67.8% | 31.7% | 27.0% | 86.1% | 16.6% | 61.4% | 24.8% | 21.5% | 86.2% |
| 651 | 24.3% | 57.9% | 24.3% | 18.5% | 72.9% | 6.4% | 63.0% | 34.7% | 24.5% | 88.5% | 16.8% | 55.6% | 26.5% | 17.6% | 87.9% |
| 652 | 44.5% | 67.1% | 24.6% | 19.4% | 73.3% | 7.8% | 69.8% | 33.3% | 23.0% | 86.8% | 25.2% | 74.0% | 25.0% | 16.6% | 88.7% |
| 653 | 32.0% | 53.3% | 26.8% | 13.0% | 72.3% | 20.2% | 60.4% | 45.7% | 23.9% | 87.9% | 27.1% | 54.4% | 31.9% | 16.3% | 88.3% |
| 654 | 22.2% | 54.8% | 19.8% | 19.4% | 69.8% | 5.5% | 61.1% | 30.9% | 31.8% | 87.8% | 19.2% | 56.7% | 22.1% | 19.8% | 85.7% |
| 655 | 30.6% | 57.9% | 25.0% | 16.4% | 69.9% | 11.0% | 66.6% | 41.1% | 22.6% | 86.2% | 21.6% | 60.1% | 27.9% | 16.5% | 84.9% |
| 656 | 45.1% | 61.4% | 27.9% | 18.6% | 68.4% | 22.6% | 60.4% | 35.2% | 22.1% | 83.8% | 39.1% | 64.1% | 33.5% | 19.9% | 85.7% |
| 657 | 38.6% | 60.5% | 31.6% | 21.2% | 67.5% | 9.8% | 61.8% | 28.4% | 21.7% | 83.0% | 25.0% | 66.5% | 27.4% | 17.4% | 83.9% |
| 658 | 29.4% | 63.9% | 24.5% | 22.4% | 71.3% | 6.3% | 64.3% | 33.4% | 22.1% | 83.9% | 22.6% | 65.5% | 27.2% | 19.2% | 85.5% |
| 659 | 29.9% | 60.3% | 29.7% | 16.5% | 68.1% | 13.3% | 64.8% | 47.7% | 22.7% | 85.2% | 22.5% | 66.2% | 37.3% | 19.3% | 86.0% |
| 660 | 40.9% | 57.9% | 28.0% | 16.8% | 67.1% | 23.1% | 59.3% | 39.6% | 28.0% | 85.0% | 36.2% | 58.3% | 29.0% | 20.3% | 83.1% |
| 661 | 38.5% | 65.1% | 32.8% | 21.0% | 71.1% | 14.3% | 64.9% | 30.9% | 28.5% | 83.9% | 26.5% | 67.7% | 28.3% | 19.8% | 84.5% |
| 662 | 35.4% | 60.0% | 33.3% | 13.8% | 70.7% | 8.2% | 60.4% | 44.0% | 22.9% | 84.6% | 25.5% | 59.2% | 34.7% | 16.5% | 85.2% |
| 663 | 27.5% | 61.7% | 24.8% | 20.7% | 69.9% | 6.6% | 66.0% | 32.8% | 22.5% | 84.8% | 23.3% | 64.5% | 27.2% | 20.9% | 84.5% |
| 664 | 28.0% | 58.5% | 32.8% | 15.4% | 66.4% | 8.0% | 63.2% | 46.0% | 24.7% | 84.5% | 23.0% | 64.8% | 35.5% | 19.7% | 84.6% |
| 665 | 37.8% | 57.4% | 33.9% | 18.3% | 65.4% | 23.9% | 57.6% | 36.6% | 30.8% | 85.8% | 35.7% | 59.2% | 32.1% | 20.6% | 84.4% |
| 666 | 33.8% | 59.3% | 40.8% | 24.9% | 65.5% | 8.1% | 63.3% | 40.5% | 33.7% | 84.2% | 29.3% | 67.5% | 34.4% | 22.1% | 85.0% |
| 667 | 27.1% | 57.7% | 35.3% | 14.7% | 66.3% | 9.7% | 60.6% | 45.6% | 35.6% | 84.2% | 22.7% | 60.9% | 37.1% | 20.9% | 83.9% |
| 668 | 22.3% | 58.1% | 30.6% | 20.4% | 65.5% | 7.2% | 62.8% | 38.8% | 31.0% | 83.6% | 22.2% | 63.8% | 30.6% | 24.0% | 83.7% |
| 669 | 28.5% | 57.3% | 35.8% | 14.2% | 65.2% | 5.8% | 63.0% | 44.0% | 31.5% | 83.6% | 21.9% | 61.9% | 33.8% | 20.9% | 83.1% |
| 670 | 47.6% | 65.2% | 48.3% | 20.1% | 71.2% | 31.8% | 62.5% | 45.8% | 37.0% | 85.4% | 43.2% | 70.2% | 48.2% | 27.6% | 87.0% |
| 671 | 29.5% | 66.2% | 43.9% | 13.1% | 74.9% | 18.2% | 61.7% | 49.6% | 30.5% | 83.2% | 27.6% | 65.4% | 47.8% | 18.9% | 86.1% |
| 672 | 23.3% | 59.7% | 35.5% | 25.4% | 67.5% | 5.7% | 59.8% | 38.2% | 32.8% | 83.0% | 20.0% | 65.7% | 36.4% | 28.8% | 83.1% |
| 673 | 29.2% | 61.6% | 42.9% | 18.9% | 68.3% | 6.0% | 62.0% | 43.5% | 31.0% | 85.4% | 24.3% | 67.0% | 38.8% | 21.9% | 85.6% |
| 674 | 42.4% | 60.8% | 42.2% | 17.5% | 69.4% | 21.4% | 53.2% | 33.5% | 33.9% | 84.7% | 34.6% | 63.4% | 37.1% | 23.8% | 84.7% |
| 675 | 37.0% | 62.6% | 45.9% | 34.7% | 69.5% | 9.8% | 67.3% | 50.0% | 45.8% | 85.9% | 27.8% | 68.3% | 47.0% | 33.3% | 85.4% |
| 676 | 25.6% | 56.6% | 37.4% | 15.9% | 63.7% | 8.8% | 56.4% | 40.1% | 31.3% | 83.1% | 25.2% | 62.6% | 36.9% | 20.4% | 84.1% |
| 677 | 23.1% | 60.4% | 36.4% | 25.9% | 67.3% | 7.9% | 64.7% | 40.4% | 30.1% | 85.0% | 23.0% | 66.6% | 38.8% | 27.3% | 84.0% |
| 678 | 30.5% | 62.5% | 44.3% | 23.8% | 70.3% | 6.1% | 63.8% | 48.3% | 28.6% | 84.5% | 25.0% | 66.2% | 43.6% | 23.2% | 85.1% |
| 679 | 51.0% | 68.5% | 52.3% | 29.8% | 74.1% | 18.1% | 56.5% | 35.0% | 38.8% | 87.0% | 41.2% | 64.4% | 37.8% | 26.8% | 85.3% |
| 680 | 45.4% | 68.9% | 54.1% | 46.2% | 75.0% | 6.0% | 67.1% | 30.2% | 31.1% | 85.9% | 36.2% | 70.1% | 37.5% | 29.6% | 87.3% |
| 681 | 21.5% | 62.8% | 40.2% | 31.3% | 69.8% | 6.9% | 56.5% | 35.0% | 39.7% | 85.2% | 21.3% | 65.1% | 34.8% | 32.4% | 85.3% |
| 682 | 30.8% | 60.7% | 44.3% | 29.7% | 67.5% | 5.7% | 62.6% | 42.5% | 39.1% | 85.3% | 30.2% | 66.7% | 40.7% | 27.1% | 85.6% |
| 683 | 42.3% | 59.9% | 45.5% | 30.5% | 65.9% | 18.6% | 56.7% | 34.3% | 35.9% | 86.0% | 40.2% | 63.9% | 37.3% | 24.8% | 83.7% |
| 684 | 40.6% | 63.9% | 50.2% | 42.4% | 69.7% | 6.4% | 71.2% | 44.5% | 37.4% | 87.8% | 30.2% | 68.2% | 35.9% | 24.6% | 84.6% |
| 685 | 35.5% | 64.3% | 48.8% | 31.6% | 70.6% | 6.5% | 52.0% | 37.9% | 25.0% | 86.0% | 31.0% | 65.9% | 37.7% | 19.1% | 86.6% |
| 686 | 28.3% | 58.4% | 39.8% | 33.3% | 66.1% | 6.5% | 56.1% | 36.8% | 40.7% | 85.5% | 28.8% | 65.1% | 32.7% | 29.0% | 83.4% |
| 687 | 40.5% | 66.7% | 53.1% | 37.5% | 73.0% | 5.5% | 66.3% | 45.0% | 35.9% | 87.3% | 34.8% | 68.9% | 37.1% | 23.7% | 88.0% |
| 688 | 41.9% | 61.8% | 43.9% | 29.5% | 68.9% | 20.4% | 55.9% | 38.9% | 31.5% | 85.9% | 38.6% | 66.1% | 40.9% | 25.0% | 85.0% |
| 689 | 35.9% | 62.3% | 45.1% | 36.9% | 68.0% | 7.7% | 64.1% | 30.8% | 31.3% | 85.5% | 25.8% | 66.6% | 34.4% | 26.9% | 83.8% |
| 690 | 30.1% | 61.5% | 44.5% | 29.3% | 69.2% | 8.6% | 49.9% | 39.0% | 39.6% | 84.8% | 25.7% | 62.1% | 37.4% | 24.5% | 82.7% |
| 691 | 26.3% | 57.4% | 37.5% | 31.3% | 65.2% | 5.2% | 45.6% | 30.0% | 31.3% | 83.5% | 21.2% | 61.1% | 29.1% | 21.7% | 81.8% |
| 692 | 30.3% | 60.6% | 42.0% | 27.6% | 67.9% | 5.5% | 58.4% | 37.4% | 40.0% | 85.8% | 25.5% | 64.7% | 34.3% | 23.6% | 83.8% |
| 693 | 39.7% | 57.7% | 41.6% | 26.7% | 64.8% | 17.9% | 53.6% | 37.8% | 35.3% | 84.9% | 34.2% | 64.6% | 39.7% | 24.5% | 84.2% |
| 694 | 40.5% | 70.7% | 57.0% | 51.7% | 75.7% | 8.6% | 77.8% | 31.7% | 37.0% | 89.9% | 22.8% | 76.9% | 39.8% | 35.1% | 89.0% |
| 695 | 35.5% | 66.6% | 50.9% | 27.2% | 72.4% | 4.4% | 48.4% | 34.9% | 29.0% | 88.7% | 24.9% | 64.5% | 32.0% | 17.7% | 84.3% |
| 696 | 24.7% | 60.1% | 39.1% | 32.9% | 67.8% | 5.1% | 46.1% | 30.4% | 36.3% | 84.4% | 17.6% | 64.9% | 29.2% | 24.4% | 85.1% |
| 697 | 21.0% | 62.1% | 43.5% | 28.0% | 68.8% | 7.4% | 67.5% | 41.2% | 31.6% | 86.6% | 15.1% | 66.7% | 41.3% | 29.9% | 83.6% |
| 698 | 39.4% | 60.8% | 44.7% | 27.1% | 67.4% | 11.8% | 40.3% | 23.4% | 31.2% | 86.8% | 30.0% | 66.0% | 39.8% | 21.8% | 85.1% |
| 699 | 32.8% | 65.0% | 50.5% | 41.6% | 70.9% | 8.6% | 70.6% | 37.3% | 34.0% | 87.6% | 18.6% | 70.8% | 40.6% | 31.7% | 85.9% |
| 700 | 24.0% | 59.0% | 45.6% | 22.7% | 66.3% | 5.3% | 45.1% | 33.7% | 21.2% | 84.6% | 16.4% | 64.2% | 38.9% | 16.8% | 84.0% |
| 701 | 25.0% | 67.9% | 42.2% | 38.8% | 74.1% | 4.5% | 69.1% | 44.6% | 40.9% | 89.0% | 15.3% | 72.4% | 35.4% | 32.3% | 89.7% |
| 702 | 22.6% | 65.7% | 46.5% | 27.8% | 71.9% | 7.6% | 64.0% | 41.0% | 41.0% | 87.9% | 16.4% | 70.7% | 44.5% | 30.2% | 88.6% |
| 703 | 38.3% | 57.1% | 42.4% | 24.9% | 63.9% | 12.7% | 50.2% | 34.4% | 29.1% | 85.8% | 30.2% | 63.6% | 37.2% | 21.0% | 84.1% |
| 704 | 28.3% | 65.1% | 51.4% | 43.2% | 72.7% | 10.4% | 75.7% | 36.8% | 45.7% | 90.8% | 19.6% | 69.6% | 41.6% | 36.3% | 88.4% |
| 705 | 19.1% | 55.9% | 40.7% | 23.9% | 64.4% | 9.6% | 59.8% | 50.4% | 33.6% | 84.8% | 16.8% | 62.0% | 40.9% | 21.8% | 82.6% |
| 706 | 19.4% | 59.9% | 39.7% | 30.0% | 67.6% | 6.7% | 44.9% | 29.1% | 37.5% | 86.4% | 15.4% | 64.7% | 31.7% | 24.6% | 85.0% |
| 707 | 21.9% | 62.1% | 44.7% | 25.5% | 69.5% | 5.5% | 62.8% | 38.9% | 34.4% | 87.6% | 16.4% | 68.3% | 37.7% | 21.2% | 86.9% |
| 708 | 40.2% | 59.1% | 41.5% | 25.0% | 65.8% | 15.7% | 49.4% | 29.0% | 27.8% | 86.2% | 37.5% | 64.9% | 34.7% | 20.8% | 85.3% |
| 709 | 29.3% | 61.0% | 45.1% | 36.7% | 67.7% | 8.7% | 63.2% | 27.7% | 26.2% | 84.3% | 17.8% | 68.7% | 30.7% | 23.7% | 86.3% |
| 710 | 23.1% | 62.3% | 46.9% | 26.1% | 69.1% | 5.3% | 49.4% | 38.7% | 24.5% | 86.8% | 16.9% | 66.5% | 42.9% | 18.9% | 87.0% |
| 711 | 18.4% | 55.4% | 35.7% | 29.9% | 62.2% | 4.9% | 42.9% | 24.4% | 28.3% | 82.4% | 13.5% | 59.5% | 27.5% | 24.5% | 81.4% |
| 712 | 23.4% | 64.2% | 47.4% | 28.3% | 71.0% | 5.5% | 56.2% | 33.6% | 24.4% | 87.7% | 15.2% | 67.8% | 38.5% | 23.5% | 88.7% |
| 713 | 40.1% | 62.0% | 44.9% | 29.8% | 69.9% | 14.9% | 42.1% | 25.0% | 33.9% | 86.6% | 34.1% | 66.0% | 40.1% | 28.2% | 87.8% |
| 714 | 34.2% | 66.7% | 51.9% | 43.1% | 72.9% | 13.9% | 76.3% | 47.4% | 34.0% | 90.1% | 22.0% | 76.6% | 41.8% | 30.5% | 90.3% |
| 715 | 23.0% | 62.4% | 47.9% | 26.9% | 69.3% | 4.8% | 51.4% | 39.2% | 22.4% | 87.6% | 15.9% | 66.5% | 43.2% | 19.0% | 87.8% |
| 716 | 22.7% | 61.0% | 42.6% | 24.9% | 68.6% | 6.6% | 64.8% | 42.0% | 43.7% | 90.9% | 15.2% | 70.6% | 42.0% | 31.9% | 90.4% |
| 717 | 44.4% | 62.2% | 44.9% | 27.7% | 69.4% | 11.0% | 39.8% | 22.2% | 29.7% | 87.9% | 40.1% | 55.7% | 28.3% | 24.2% | 89.7% |
| 718 | 41.9% | 66.9% | 48.8% | 39.7% | 73.1% | 6.5% | 65.9% | 30.1% | 22.0% | 85.6% | 22.1% | 73.7% | 27.7% | 18.3% | 88.2% |
| 719 | 24.7% | 63.0% | 47.7% | 23.5% | 69.7% | 5.5% | 54.8% | 48.4% | 29.4% | 89.8% | 20.0% | 58.0% | 42.8% | 19.6% | 89.3% |
| 720 | 21.6% | 59.5% | 36.4% | 30.0% | 65.8% | 5.6% | 50.7% | 33.1% | 42.3% | 86.3% | 16.2% | 57.0% | 31.8% | 38.7% | 87.7% |
| 721 | 24.4% | 61.5% | 42.3% | 23.1% | 67.7% | 3.5% | 54.4% | 32.3% | 40.7% | 90.1% | 16.9% | 56.4% | 27.6% | 26.3% | 90.6% |
| 722 | 44.8% | 62.0% | 42.3% | 26.2% | 69.2% | 21.4% | 59.2% | 42.8% | 34.6% | 90.4% | 47.9% | 64.2% | 39.9% | 28.3% | 90.0% |
| 723 | 34.6% | 62.9% | 41.4% | 31.4% | 69.8% | 9.3% | 66.1% | 21.8% | 26.7% | 85.9% | 17.4% | 72.5% | 25.0% | 23.3% | 88.0% |
| 724 | 29.6% | 66.8% | 45.2% | 20.5% | 74.0% | 5.5% | 40.0% | 37.2% | 39.6% | 91.8% | 21.5% | 54.2% | 34.6% | 24.0% | 91.6% |
| 725 | 21.7% | 62.5% | 35.2% | 29.6% | 69.7% | 5.6% | 62.9% | 44.9% | 45.4% | 88.4% | 19.4% | 66.2% | 44.4% | 44.7% | 87.8% |
| 726 | 26.1% | 61.0% | 37.8% | 23.7% | 68.4% | 4.4% | 53.4% | 32.2% | 27.1% | 86.2% | 20.6% | 55.9% | 30.1% | 22.1% | 87.8% |
| 727 | 50.9% | 64.2% | 38.8% | 21.1% | 69.8% | 30.1% | 46.7% | 34.9% | 47.8% | 91.3% | 57.6% | 65.2% | 37.5% | 34.2% | 90.0% |
| 728 | 46.7% | 69.1% | 42.3% | 33.0% | 74.2% | 8.0% | 78.4% | 40.2% | 33.6% | 89.9% | 28.0% | 80.8% | 30.8% | 21.8% | 90.6% |
| 729 | 29.1% | 59.7% | 40.9% | 18.6% | 67.4% | 5.6% | 45.4% | 37.6% | 32.4% | 87.1% | 19.9% | 50.4% | 32.9% | 23.4% | 85.1% |
| 730 | 26.5% | 63.2% | 33.3% | 29.9% | 69.0% | 4.4% | 58.4% | 37.0% | 40.6% | 90.3% | 19.3% | 63.8% | 30.2% | 33.1% | 90.8% |
| 731 | 35.7% | 69.1% | 40.7% | 21.3% | 74.5% | 10.7% | 77.5% | 68.2% | 30.3% | 92.1% | 27.7% | 74.8% | 59.2% | 26.3% | 90.5% |
| 732 | 43.5% | 59.7% | 35.4% | 20.3% | 66.1% | 21.6% | 55.2% | 42.5% | 33.8% | 86.8% | 46.0% | 62.9% | 40.9% | 26.8% | 86.7% |
| 733 | 41.2% | 62.8% | 37.3% | 30.1% | 69.2% | 9.6% | 79.6% | 46.1% | 39.2% | 90.6% | 26.1% | 77.6% | 36.2% | 26.1% | 88.8% |
| 734 | 38.6% | 65.5% | 44.3% | 17.6% | 72.5% | 10.9% | 71.8% | 61.3% | 38.7% | 92.0% | 30.9% | 67.9% | 47.0% | 22.3% | 90.5% |
| 735 | 25.7% | 62.7% | 34.8% | 30.0% | 68.7% | 4.7% | 45.4% | 30.2% | 43.5% | 87.4% | 17.1% | 57.1% | 26.7% | 34.8% | 88.0% |
| 736 | 34.0% | 63.9% | 35.8% | 20.6% | 69.7% | 5.7% | 69.1% | 42.3% | 26.4% | 87.8% | 29.5% | 67.2% | 35.5% | 20.4% | 85.8% |
| 737 | 51.1% | 65.8% | 38.9% | 23.7% | 72.9% | 43.2% | 70.6% | 61.4% | 36.8% | 89.9% | 54.9% | 75.4% | 59.4% | 30.0% | 89.6% |
| 738 | 42.5% | 65.7% | 36.0% | 31.3% | 72.3% | 6.4% | 71.6% | 27.0% | 29.3% | 87.3% | 28.9% | 76.8% | 27.2% | 22.5% | 89.2% |
| 739 | 33.1% | 66.0% | 43.0% | 15.8% | 72.2% | 4.6% | 54.5% | 43.5% | 22.5% | 91.3% | 23.8% | 55.3% | 36.5% | 15.2% | 91.8% |
| 740 | 27.3% | 64.0% | 35.1% | 31.4% | 71.4% | 8.4% | 62.5% | 44.0% | 47.6% | 88.5% | 22.4% | 68.2% | 39.7% | 41.2% | 89.0% |
| 741 | 36.3% | 65.6% | 36.4% | 21.7% | 72.5% | 4.5% | 68.4% | 49.1% | 30.2% | 89.8% | 27.0% | 69.5% | 45.8% | 23.6% | 89.4% |
| 742 | 42.5% | 61.2% | 30.2% | 20.7% | 71.6% | 23.2% | 56.6% | 27.8% | 21.6% | 86.9% | 34.0% | 64.7% | 31.1% | 24.6% | 89.0% |
| 743 | 33.3% | 64.6% | 34.4% | 29.5% | 71.7% | 6.5% | 73.9% | 39.8% | 28.9% | 88.5% | 27.4% | 75.2% | 32.2% | 21.6% | 89.5% |
| 744 | 25.9% | 59.7% | 36.8% | 18.0% | 70.1% | 6.4% | 49.5% | 37.7% | 24.4% | 87.4% | 18.5% | 60.9% | 37.0% | 19.7% | 87.0% |
| 745 | 22.5% | 58.6% | 30.7% | 28.3% | 66.6% | 4.5% | 64.1% | 30.2% | 28.1% | 86.5% | 18.3% | 67.7% | 28.5% | 24.8% | 85.8% |
| 746 | 22.4% | 61.3% | 31.3% | 20.5% | 69.7% | 11.9% | 67.4% | 30.9% | 20.7% | 88.2% | 18.4% | 69.0% | 33.4% | 21.7% | 88.7% |
| 747 | 24.7% | 76.5% | 20.8% | 9.0% | 81.4% | 5.9% | 58.5% | 18.3% | 12.9% | 88.1% | 14.0% | 72.4% | 19.5% | 11.3% | 90.6% |
| 748 | 12.1% | 76.3% | 23.6% | 12.4% | 82.4% | 6.1% | 47.6% | 46.6% | 48.4% | 87.1% | 12.9% | 53.2% | 40.1% | 43.7% | 88.9% |
| 749 | 19.6% | 72.1% | 23.5% | 8.1% | 80.9% | 4.9% | 33.3% | 17.5% | 16.8% | 86.9% | 11.6% | 40.4% | 17.9% | 12.9% | 90.0% |
| 750 | 49.4% | 73.1% | 26.1% | 9.3% | 85.0% | 18.2% | 42.2% | 25.6% | 15.4% | 90.2% | 28.9% | 44.2% | 21.5% | 13.0% | 91.1% |
| 751 | 25.7% | 68.8% | 26.1% | 14.5% | 74.3% | 7.6% | 64.6% | 29.5% | 17.6% | 86.2% | 13.9% | 69.6% | 26.1% | 15.3% | 87.9% |
| 752 | 12.4% | 70.7% | 22.0% | 12.8% | 78.0% | 5.4% | 45.8% | 31.6% | 32.6% | 86.0% | 12.4% | 49.8% | 22.2% | 20.8% | 87.0% |
| 753 | 19.2% | 61.9% | 22.7% | 11.7% | 76.2% | 5.5% | 48.1% | 28.0% | 19.8% | 87.1% | 12.5% | 48.9% | 24.5% | 15.3% | 88.3% |
| 754 | 46.3% | 61.2% | 33.7% | 21.2% | 73.0% | 15.0% | 39.2% | 22.8% | 17.0% | 82.5% | 30.2% | 47.3% | 25.0% | 15.5% | 85.3% |
| 755 | 22.4% | 66.5% | 33.9% | 24.8% | 72.4% | 6.5% | 65.1% | 20.0% | 15.2% | 83.3% | 16.2% | 68.3% | 23.0% | 15.0% | 86.5% |
| 756 | 20.0% | 60.9% | 42.0% | 15.9% | 74.1% | 9.5% | 54.5% | 36.6% | 18.8% | 83.3% | 17.4% | 50.2% | 30.9% | 16.9% | 85.3% |
| 757 | 16.1% | 82.1% | 33.0% | 33.6% | 87.0% | 16.0% | 65.6% | 49.7% | 34.0% | 92.0% | 17.2% | 67.4% | 39.9% | 27.8% | 92.9% |
| 758 | 14.3% | 69.6% | 31.8% | 13.3% | 77.0% | 5.9% | 49.0% | 29.0% | 18.3% | 85.4% | 13.3% | 47.3% | 23.2% | 14.8% | 85.3% |
| 759 | 47.6% | 64.1% | 26.1% | 28.2% | 83.0% | 23.7% | 48.1% | 23.6% | 17.0% | 91.0% | 25.9% | 47.4% | 20.5% | 15.2% | 90.9% |
| 760 | 20.5% | 68.6% | 22.1% | 28.5% | 77.8% | 14.9% | 70.5% | 21.4% | 13.3% | 87.4% | 17.2% | 71.0% | 21.1% | 14.5% | 88.7% |
| 761 | 17.0% | 55.4% | 41.1% | 18.1% | 71.4% | 6.7% | 39.7% | 27.3% | 15.3% | 81.6% | 15.3% | 46.9% | 26.3% | 14.0% | 82.1% |
| 762 | 14.9% | 50.8% | 24.7% | 35.5% | 76.3% | 8.2% | 62.2% | 17.5% | 17.0% | 89.0% | 14.5% | 47.7% | 18.9% | 17.1% | 88.6% |
| 763 | 20.7% | 67.2% | 27.0% | 25.2% | 83.2% | 7.0% | 66.8% | 42.1% | 25.3% | 90.6% | 13.9% | 58.8% | 29.4% | 18.5% | 90.6% |
| 764 | 49.0% | 56.7% | 21.1% | 19.3% | 78.5% | 40.8% | 72.8% | 41.7% | 15.3% | 91.3% | 49.9% | 59.1% | 26.0% | 13.8% | 91.4% |
| 765 | 38.0% | 68.2% | 20.0% | 19.7% | 75.5% | 30.8% | 72.4% | 17.2% | 13.3% | 86.8% | 27.0% | 74.9% | 20.0% | 14.8% | 88.8% |
| 766 | 23.3% | 52.1% | 29.1% | 13.7% | 70.6% | 6.9% | 45.4% | 26.8% | 16.5% | 84.1% | 17.8% | 48.9% | 25.1% | 15.1% | 84.2% |
| 767 | 25.2% | 53.3% | 21.7% | 28.3% | 72.2% | 5.4% | 71.0% | 31.6% | 23.7% | 89.3% | 22.1% | 60.6% | 25.1% | 19.3% | 86.7% |
| 768 | 20.8% | 61.8% | 27.1% | 24.5% | 77.3% | 20.3% | 70.6% | 52.9% | 17.4% | 89.1% | 18.1% | 66.8% | 40.9% | 16.9% | 90.1% |
| 769 | 52.0% | 65.4% | 25.1% | 18.9% | 75.0% | 34.5% | 61.6% | 33.7% | 18.4% | 88.1% | 41.6% | 61.7% | 29.0% | 16.3% | 87.9% |
| 770 | 35.0% | 63.0% | 23.9% | 20.2% | 70.9% | 10.7% | 69.4% | 28.0% | 16.9% | 85.6% | 23.3% | 70.4% | 24.8% | 16.9% | 87.5% |
| 771 | 31.7% | 55.5% | 27.3% | 13.4% | 72.7% | 8.7% | 50.6% | 35.0% | 17.9% | 84.1% | 24.6% | 55.8% | 32.0% | 16.2% | 86.3% |
| 772 | 25.2% | 59.6% | 27.3% | 29.1% | 72.8% | 7.1% | 67.5% | 33.9% | 23.5% | 85.6% | 19.7% | 65.4% | 27.8% | 20.2% | 86.5% |
| 773 | 26.4% | 65.6% | 23.9% | 20.4% | 80.9% | 26.1% | 75.3% | 41.3% | 33.7% | 91.7% | 25.4% | 71.2% | 33.4% | 25.0% | 92.7% |
| 774 | 45.9% | 60.8% | 25.3% | 16.9% | 70.4% | 31.0% | 67.5% | 51.4% | 25.0% | 86.1% | 43.8% | 66.5% | 39.3% | 18.7% | 87.0% |
| 775 | 34.5% | 62.0% | 25.6% | 20.0% | 68.5% | 18.6% | 66.5% | 25.8% | 15.7% | 83.9% | 25.3% | 69.0% | 24.8% | 15.1% | 83.9% |
| 776 | 30.7% | 59.5% | 29.6% | 14.1% | 70.9% | 11.6% | 55.8% | 40.5% | 18.1% | 85.4% | 25.4% | 62.2% | 34.1% | 16.5% | 87.3% |
| 777 | 27.1% | 65.4% | 24.3% | 24.6% | 75.1% | 5.1% | 72.0% | 34.3% | 27.0% | 88.8% | 21.2% | 65.7% | 24.7% | 20.0% | 88.6% |
| 778 | 31.6% | 59.8% | 26.2% | 16.4% | 69.6% | 11.0% | 66.9% | 43.2% | 16.6% | 86.1% | 21.2% | 66.1% | 32.5% | 15.8% | 86.8% |
| 779 | 45.7% | 62.1% | 27.0% | 15.8% | 69.4% | 26.0% | 65.4% | 44.3% | 18.6% | 86.4% | 37.5% | 64.7% | 31.7% | 17.7% | 85.2% |
| 780 | 42.5% | 64.0% | 31.3% | 19.7% | 70.5% | 12.9% | 66.7% | 31.7% | 21.1% | 84.5% | 27.6% | 69.7% | 29.5% | 18.0% | 85.8% |
| 781 | 28.4% | 57.2% | 30.4% | 14.8% | 66.5% | 11.4% | 64.1% | 46.7% | 23.8% | 83.9% | 22.4% | 64.9% | 39.2% | 18.5% | 84.5% |
| 782 | 27.7% | 61.3% | 24.0% | 20.4% | 69.8% | 8.1% | 63.1% | 24.0% | 19.2% | 84.8% | 19.9% | 61.0% | 23.9% | 18.5% | 84.0% |
| 783 | 23.5% | 60.9% | 28.8% | 20.2% | 70.1% | 29.9% | 62.7% | 27.9% | 13.9% | 87.1% | 29.3% | 65.4% | 27.9% | 17.5% | 88.2% |
| 784 | 41.4% | 60.0% | 31.9% | 16.5% | 67.9% | 19.2% | 59.2% | 40.8% | 19.8% | 83.0% | 38.8% | 64.3% | 35.4% | 17.8% | 84.8% |
| 785 | 37.5% | 61.3% | 28.4% | 17.4% | 68.8% | 12.4% | 66.7% | 33.8% | 23.4% | 84.9% | 30.3% | 68.2% | 26.9% | 17.6% | 86.5% |
| 786 | 35.3% | 66.7% | 39.0% | 10.8% | 74.1% | 11.4% | 57.4% | 33.5% | 15.6% | 82.4% | 23.4% | 60.3% | 32.7% | 12.8% | 82.1% |
| 787 | 20.3% | 58.3% | 26.4% | 20.3% | 65.5% | 7.9% | 60.8% | 30.2% | 25.2% | 81.6% | 19.6% | 62.3% | 26.9% | 21.6% | 82.6% |
| 788 | 31.0% | 60.6% | 28.3% | 16.3% | 69.2% | 6.8% | 62.4% | 47.3% | 20.0% | 84.0% | 24.5% | 66.9% | 38.4% | 18.3% | 85.4% |
| 789 | 39.1% | 58.8% | 34.5% | 17.0% | 66.1% | 23.7% | 60.1% | 39.7% | 26.6% | 85.3% | 35.6% | 65.6% | 37.9% | 20.9% | 86.1% |
| 790 | 35.8% | 59.0% | 37.6% | 20.8% | 65.6% | 8.2% | 63.9% | 42.0% | 31.1% | 83.5% | 29.2% | 65.8% | 35.6% | 21.3% | 83.4% |
| 791 | 29.0% | 57.5% | 39.0% | 16.0% | 65.1% | 6.6% | 54.7% | 36.7% | 23.0% | 82.7% | 27.4% | 63.2% | 35.3% | 18.4% | 84.0% |
| 792 | 21.2% | 58.0% | 28.4% | 18.5% | 67.5% | 8.4% | 56.3% | 38.3% | 31.4% | 79.1% | 18.3% | 61.8% | 37.3% | 28.9% | 81.6% |
| 793 | 27.2% | 60.0% | 39.8% | 18.7% | 67.8% | 10.9% | 60.7% | 38.6% | 21.4% | 85.7% | 21.8% | 65.2% | 34.8% | 20.2% | 86.7% |
| 794 | 41.3% | 61.9% | 43.9% | 23.5% | 69.4% | 15.0% | 59.1% | 42.8% | 28.4% | 84.1% | 40.9% | 68.6% | 44.9% | 23.8% | 86.3% |
| 795 | 44.3% | 67.4% | 49.7% | 26.1% | 74.6% | 14.4% | 65.7% | 35.2% | 33.6% | 84.0% | 31.2% | 70.4% | 40.3% | 26.3% | 86.2% |
| 796 | 24.0% | 54.8% | 38.5% | 17.9% | 63.8% | 8.6% | 46.7% | 31.1% | 22.1% | 81.8% | 23.9% | 60.3% | 36.6% | 19.0% | 82.2% |
| 797 | 24.7% | 65.1% | 33.6% | 21.3% | 72.0% | 7.2% | 61.1% | 27.6% | 24.6% | 83.8% | 23.3% | 67.2% | 31.7% | 25.3% | 85.4% |
| 798 | 25.4% | 58.0% | 40.9% | 19.2% | 65.4% | 11.5% | 59.6% | 40.1% | 23.4% | 83.0% | 24.2% | 64.2% | 40.6% | 22.0% | 82.8% |
| 799 | 39.5% | 59.8% | 42.9% | 24.4% | 67.2% | 13.2% | 56.5% | 39.4% | 29.1% | 84.2% | 37.8% | 65.6% | 44.8% | 25.0% | 84.8% |
| 800 | 33.4% | 57.4% | 41.9% | 33.6% | 65.5% | 8.7% | 61.9% | 36.3% | 31.3% | 84.8% | 29.9% | 63.4% | 37.2% | 28.9% | 83.5% |
| 801 | 28.7% | 62.3% | 43.3% | 20.2% | 70.7% | 7.5% | 61.4% | 45.3% | 31.8% | 84.5% | 25.8% | 65.5% | 41.8% | 23.6% | 85.9% |
| 802 | 23.3% | 62.3% | 38.3% | 26.3% | 70.0% | 9.8% | 61.4% | 48.0% | 46.1% | 85.0% | 22.3% | 66.2% | 42.9% | 37.0% | 86.4% |
| 803 | 29.0% | 62.3% | 44.1% | 24.2% | 69.2% | 6.2% | 65.9% | 47.9% | 27.8% | 85.8% | 27.2% | 69.3% | 46.1% | 24.3% | 86.5% |
| 804 | 35.9% | 60.0% | 44.3% | 30.4% | 66.2% | 28.4% | 56.4% | 27.5% | 29.0% | 84.3% | 34.3% | 65.1% | 39.1% | 28.6% | 84.3% |
| 805 | 37.6% | 63.9% | 49.1% | 40.6% | 70.7% | 6.8% | 66.9% | 47.2% | 40.6% | 86.3% | 32.2% | 68.6% | 43.0% | 31.5% | 85.6% |
| 806 | 35.6% | 70.1% | 56.3% | 34.5% | 75.9% | 10.8% | 55.3% | 41.2% | 27.1% | 88.4% | 32.5% | 69.6% | 45.5% | 24.5% | 86.3% |
| 807 | 29.1% | 61.5% | 42.0% | 33.0% | 68.2% | 5.8% | 61.6% | 34.0% | 30.6% | 84.1% | 30.5% | 67.6% | 35.1% | 28.2% | 84.4% |
| 808 | 30.8% | 61.5% | 45.1% | 28.5% | 67.9% | 7.9% | 66.4% | 45.4% | 28.1% | 87.0% | 27.8% | 67.0% | 42.1% | 22.5% | 85.1% |
| 809 | 41.9% | 61.3% | 45.4% | 31.6% | 68.0% | 21.5% | 65.9% | 44.7% | 30.8% | 88.2% | 38.5% | 66.9% | 41.2% | 25.8% | 86.5% |
| 810 | 43.7% | 66.0% | 52.5% | 44.9% | 71.9% | 8.4% | 71.9% | 31.3% | 32.7% | 88.5% | 36.2% | 72.3% | 43.5% | 35.7% | 88.1% |
| 811 | 36.0% | 64.9% | 48.9% | 29.5% | 71.0% | 5.0% | 53.5% | 44.1% | 31.2% | 89.4% | 29.2% | 67.3% | 38.3% | 20.5% | 85.1% |
| 812 | 24.8% | 61.0% | 38.8% | 32.4% | 68.0% | 5.6% | 53.2% | 24.9% | 25.2% | 86.5% | 20.1% | 68.0% | 26.5% | 22.1% | 86.2% |
| 813 | 33.4% | 62.4% | 46.4% | 29.5% | 68.9% | 7.0% | 72.1% | 48.8% | 28.9% | 88.4% | 27.8% | 69.6% | 44.4% | 23.9% | 85.5% |
| 814 | 41.0% | 59.7% | 43.2% | 30.0% | 67.9% | 25.4% | 53.9% | 32.7% | 32.2% | 85.3% | 40.7% | 67.1% | 39.5% | 27.8% | 86.6% |
| 815 | 48.3% | 71.6% | 57.9% | 50.6% | 76.9% | 5.4% | 71.9% | 30.6% | 24.4% | 87.8% | 32.5% | 72.7% | 41.8% | 31.8% | 88.2% |
| 816 | 29.8% | 60.9% | 44.1% | 29.8% | 68.6% | 8.0% | 49.7% | 36.7% | 28.1% | 86.0% | 27.5% | 64.6% | 41.3% | 22.6% | 84.4% |
| 817 | 29.2% | 60.8% | 42.3% | 36.2% | 68.3% | 8.3% | 50.9% | 23.6% | 27.1% | 85.2% | 22.9% | 64.6% | 33.0% | 29.3% | 84.1% |
| 818 | 32.7% | 67.2% | 50.5% | 33.2% | 73.5% | 10.2% | 68.2% | 35.1% | 25.8% | 88.4% | 30.0% | 70.2% | 42.3% | 25.1% | 86.0% |
| 819 | 36.5% | 60.1% | 43.6% | 27.4% | 67.8% | 22.7% | 53.1% | 29.6% | 26.9% | 86.8% | 32.7% | 67.6% | 38.4% | 24.2% | 86.7% |
| 820 | 37.5% | 66.6% | 52.5% | 43.0% | 72.5% | 7.4% | 67.1% | 28.9% | 20.3% | 86.7% | 23.0% | 72.4% | 42.5% | 28.8% | 88.0% |
| 821 | 29.9% | 60.8% | 45.5% | 27.9% | 68.0% | 6.3% | 63.5% | 52.1% | 35.6% | 87.6% | 17.9% | 65.5% | 43.2% | 21.8% | 83.8% |
| 822 | 22.2% | 61.1% | 43.5% | 25.9% | 68.9% | 6.3% | 65.6% | 39.4% | 26.6% | 87.5% | 16.4% | 67.3% | 39.0% | 22.5% | 85.3% |
| 823 | 42.1% | 63.4% | 46.2% | 27.8% | 70.4% | 15.4% | 49.9% | 30.6% | 33.8% | 87.9% | 34.6% | 68.3% | 41.8% | 23.0% | 86.3% |
| 824 | 32.4% | 64.3% | 49.2% | 39.9% | 70.4% | 7.7% | 69.7% | 28.1% | 23.1% | 86.3% | 19.0% | 69.9% | 39.6% | 28.4% | 87.2% |
| 825 | 23.0% | 62.8% | 48.1% | 30.6% | 69.9% | 6.4% | 66.0% | 47.7% | 26.7% | 87.3% | 17.2% | 67.1% | 46.3% | 21.8% | 86.2% |
| 826 | 21.8% | 60.4% | 41.8% | 34.2% | 68.3% | 4.1% | 42.7% | 19.4% | 21.5% | 82.9% | 14.7% | 63.7% | 30.2% | 23.6% | 84.7% |
| 827 | 22.2% | 61.5% | 43.7% | 28.2% | 69.3% | 13.9% | 59.4% | 25.8% | 19.3% | 84.8% | 19.3% | 68.0% | 39.1% | 26.4% | 86.5% |
| 828 | 34.9% | 60.9% | 44.0% | 26.8% | 67.6% | 35.6% | 55.3% | 28.1% | 22.6% | 86.0% | 38.4% | 66.4% | 42.8% | 23.9% | 85.4% |
| 829 | 28.7% | 64.4% | 49.7% | 41.1% | 70.7% | 6.1% | 68.3% | 25.7% | 20.7% | 86.2% | 18.0% | 71.1% | 42.5% | 29.2% | 86.4% |
| 830 | 23.5% | 60.1% | 42.3% | 25.1% | 68.4% | 11.5% | 42.9% | 30.2% | 24.3% | 87.0% | 18.4% | 66.1% | 40.0% | 22.0% | 86.1% |
| 831 | 22.4% | 63.0% | 42.3% | 33.8% | 70.5% | 5.4% | 59.7% | 32.4% | 30.6% | 86.8% | 17.5% | 66.5% | 34.1% | 25.3% | 85.4% |
| 832 | 25.4% | 68.5% | 49.1% | 30.2% | 74.4% | 8.2% | 73.4% | 42.0% | 35.3% | 92.2% | 13.7% | 76.2% | 46.5% | 31.8% | 90.7% |
| 833 | 37.5% | 61.4% | 41.3% | 26.7% | 68.4% | 17.4% | 53.5% | 39.0% | 32.9% | 88.7% | 40.3% | 71.4% | 44.2% | 23.6% | 87.9% |
| 834 | 29.9% | 59.8% | 45.6% | 37.1% | 65.8% | 7.4% | 64.4% | 23.7% | 18.0% | 84.2% | 17.9% | 68.8% | 38.3% | 27.3% | 85.7% |
| 835 | 23.5% | 59.8% | 44.6% | 25.3% | 66.9% | 5.7% | 55.5% | 42.7% | 26.6% | 87.1% | 17.0% | 67.2% | 45.0% | 20.4% | 87.7% |
| 836 | 20.7% | 62.1% | 41.1% | 32.6% | 68.3% | 9.4% | 48.6% | 29.7% | 38.1% | 86.4% | 18.9% | 69.0% | 35.6% | 31.1% | 87.8% |
| 837 | 22.4% | 62.7% | 46.9% | 29.7% | 70.0% | 5.4% | 61.3% | 41.0% | 28.7% | 87.3% | 15.9% | 72.2% | 45.1% | 23.4% | 88.1% |
| 838 | 35.9% | 62.8% | 44.8% | 28.5% | 70.0% | 34.9% | 56.4% | 30.3% | 25.4% | 87.8% | 42.6% | 69.7% | 43.1% | 26.7% | 89.0% |
| 839 | 29.9% | 62.1% | 47.0% | 37.0% | 67.4% | 8.2% | 66.6% | 20.8% | 18.1% | 84.6% | 18.3% | 72.8% | 29.7% | 23.5% | 86.5% |
| 840 | 18.9% | 61.9% | 45.1% | 30.9% | 69.8% | 13.6% | 44.1% | 27.2% | 19.0% | 85.9% | 21.5% | 65.1% | 44.0% | 24.0% | 85.8% |
| 841 | 20.5% | 61.8% | 43.7% | 35.4% | 69.7% | 7.3% | 56.6% | 35.4% | 41.8% | 87.2% | 18.3% | 67.2% | 41.7% | 42.2% | 88.3% |
| 842 | 23.3% | 63.4% | 46.0% | 31.1% | 70.1% | 5.2% | 60.1% | 37.6% | 23.2% | 86.0% | 15.3% | 68.3% | 41.7% | 23.3% | 87.2% |
| 843 | 46.9% | 64.2% | 45.2% | 28.9% | 70.7% | 24.8% | 48.6% | 31.6% | 26.5% | 90.0% | 48.7% | 63.5% | 35.3% | 23.4% | 90.3% |
| 844 | 33.0% | 62.0% | 44.3% | 34.6% | 69.0% | 8.3% | 70.2% | 26.9% | 24.9% | 87.8% | 21.1% | 74.4% | 30.4% | 25.4% | 89.5% |
| 845 | 24.2% | 60.8% | 42.5% | 23.8% | 68.8% | 11.4% | 42.2% | 30.3% | 25.1% | 86.4% | 18.8% | 56.3% | 38.2% | 21.3% | 86.2% |
| 846 | 39.9% | 60.8% | 39.6% | 25.1% | 68.7% | 31.5% | 55.3% | 31.6% | 27.8% | 88.5% | 46.7% | 63.7% | 33.8% | 26.9% | 89.1% |
| 847 | 36.0% | 63.2% | 46.6% | 37.7% | 69.6% | 5.2% | 67.5% | 23.7% | 19.7% | 85.8% | 20.0% | 73.0% | 25.6% | 18.9% | 86.9% |
| 848 | 29.1% | 65.2% | 48.1% | 19.8% | 72.4% | 5.1% | 48.6% | 45.2% | 24.1% | 88.6% | 17.8% | 59.1% | 44.7% | 17.7% | 90.5% |
| 849 | 22.0% | 62.1% | 33.4% | 27.7% | 68.8% | 5.0% | 62.3% | 35.6% | 33.9% | 87.6% | 16.9% | 65.8% | 33.5% | 27.5% | 87.0% |
| 850 | 28.2% | 65.4% | 43.7% | 26.2% | 71.6% | 13.2% | 63.2% | 29.1% | 20.9% | 91.1% | 19.9% | 68.3% | 30.0% | 20.9% | 91.7% |
| 851 | 60.4% | 74.1% | 45.2% | 25.8% | 79.3% | 44.5% | 63.3% | 54.1% | 54.6% | 91.6% | 60.0% | 75.6% | 54.5% | 47.3% | 91.4% |
| 852 | 38.9% | 62.3% | 40.3% | 31.4% | 69.0% | 8.7% | 71.1% | 21.7% | 20.5% | 86.8% | 22.6% | 73.9% | 24.4% | 19.9% | 88.1% |
| 853 | 33.4% | 72.4% | 52.3% | 21.9% | 78.9% | 7.5% | 39.5% | 28.1% | 15.6% | 87.0% | 18.6% | 51.9% | 30.9% | 14.5% | 90.1% |
| 854 | 25.2% | 66.5% | 36.9% | 33.5% | 73.3% | 4.8% | 45.0% | 17.6% | 24.9% | 86.0% | 17.3% | 59.4% | 24.7% | 30.1% | 88.6% |
| 855 | 35.5% | 72.8% | 44.0% | 21.9% | 77.7% | 4.6% | 58.9% | 32.4% | 22.7% | 93.7% | 16.8% | 63.1% | 29.4% | 19.6% | 93.3% |
| 856 | 47.5% | 65.4% | 40.6% | 21.6% | 72.0% | 19.2% | 59.4% | 42.9% | 31.9% | 88.9% | 50.5% | 70.6% | 45.3% | 26.3% | 89.8% |
| 857 | 45.6% | 68.5% | 42.3% | 32.5% | 74.4% | 9.4% | 72.5% | 24.1% | 19.6% | 87.0% | 24.3% | 72.8% | 27.4% | 19.5% | 86.7% |
| 858 | 26.5% | 56.0% | 37.0% | 16.5% | 63.4% | 6.5% | 40.2% | 28.3% | 20.3% | 82.8% | 21.1% | 57.5% | 33.7% | 17.7% | 83.9% |
| 859 | 24.2% | 60.8% | 36.2% | 31.0% | 68.3% | 9.0% | 57.2% | 22.4% | 26.0% | 86.6% | 19.7% | 65.6% | 27.9% | 28.5% | 87.5% |
| 860 | 32.7% | 63.6% | 37.2% | 21.0% | 70.3% | 8.8% | 59.4% | 27.8% | 26.2% | 87.2% | 25.6% | 63.6% | 30.5% | 21.6% | 86.9% |
| 861 | 50.1% | 67.3% | 41.1% | 24.6% | 74.6% | 23.5% | 53.3% | 35.0% | 30.5% | 90.4% | 53.8% | 68.5% | 40.6% | 25.7% | 92.8% |
| 862 | 43.0% | 64.5% | 41.9% | 34.4% | 70.6% | 7.1% | 75.8% | 25.2% | 22.6% | 90.1% | 29.2% | 77.8% | 25.5% | 20.1% | 89.7% |
| 863 | 37.0% | 70.0% | 50.0% | 20.9% | 75.7% | 6.3% | 36.9% | 29.0% | 15.3% | 88.1% | 21.2% | 63.0% | 39.8% | 16.3% | 90.4% |
| 864 | 28.1% | 65.0% | 34.5% | 31.6% | 71.9% | 7.4% | 59.2% | 32.8% | 38.1% | 88.5% | 21.6% | 67.9% | 34.5% | 32.6% | 88.4% |
| 865 | 31.1% | 61.9% | 37.9% | 21.8% | 68.9% | 7.6% | 60.0% | 34.2% | 23.9% | 86.0% | 21.1% | 65.6% | 34.5% | 21.3% | 87.5% |
| 866 | 41.3% | 60.1% | 33.1% | 21.8% | 69.5% | 29.5% | 57.9% | 33.0% | 25.5% | 88.1% | 45.1% | 67.5% | 36.4% | 23.6% | 88.6% |
| 867 | 34.1% | 63.3% | 35.6% | 29.2% | 69.7% | 9.2% | 71.9% | 27.6% | 22.7% | 88.0% | 24.3% | 75.5% | 29.7% | 22.9% | 89.4% |
| 868 | 25.0% | 59.1% | 37.7% | 17.0% | 69.5% | 5.1% | 46.5% | 33.1% | 22.1% | 87.0% | 22.6% | 61.6% | 34.1% | 15.2% | 86.6% |
| 869 | 23.8% | 62.0% | 31.6% | 29.4% | 70.9% | 5.5% | 56.5% | 33.5% | 40.0% | 87.8% | 21.4% | 67.8% | 31.6% | 32.4% | 89.4% |
| 870 | 27.4% | 64.3% | 38.2% | 25.1% | 73.2% | 6.9% | 62.4% | 31.5% | 21.1% | 89.3% | 22.8% | 66.7% | 32.1% | 20.9% | 89.8% |
| 871 | 43.8% | 77.8% | 29.7% | 6.8% | 85.0% | 23.0% | 31.7% | 18.5% | 14.8% | 90.1% | 26.2% | 49.0% | 19.5% | 11.1% | 91.5% |
| 872 | 27.5% | 73.1% | 22.7% | 11.7% | 78.8% | 8.9% | 66.5% | 26.1% | 20.3% | 86.2% | 16.9% | 72.9% | 24.2% | 16.5% | 88.9% |
| 873 | 16.7% | 76.1% | 44.3% | 6.5% | 86.5% | 4.0% | 23.6% | 17.8% | 12.3% | 89.1% | 12.4% | 38.5% | 22.5% | 10.9% | 91.3% |
| 874 | 10.6% | 82.7% | 16.2% | 6.9% | 87.4% | 3.5% | 23.3% | 10.4% | 16.4% | 89.0% | 9.4% | 49.5% | 14.0% | 14.2% | 91.4% |
| 875 | 27.9% | 69.9% | 27.0% | 9.0% | 82.1% | 4.9% | 33.5% | 17.3% | 15.4% | 85.9% | 14.1% | 50.1% | 22.2% | 15.8% | 89.7% |
| 876 | 42.7% | 70.5% | 28.2% | 11.4% | 78.3% | 21.7% | 40.3% | 26.3% | 18.5% | 86.5% | 26.0% | 49.9% | 24.2% | 15.0% | 88.8% |
| 877 | 19.0% | 71.0% | 28.1% | 13.3% | 78.9% | 5.6% | 52.5% | 18.9% | 13.5% | 85.8% | 13.5% | 69.4% | 20.7% | 13.6% | 90.0% |
| 878 | 21.0% | 64.4% | 41.0% | 10.8% | 75.8% | 6.4% | 38.3% | 29.3% | 17.9% | 82.6% | 14.5% | 43.6% | 28.3% | 14.4% | 84.9% |
| 879 | 14.2% | 71.6% | 21.3% | 11.9% | 78.1% | 5.2% | 35.8% | 18.5% | 22.2% | 86.6% | 11.0% | 47.6% | 17.5% | 15.2% | 86.5% |
| 880 | 17.0% | 78.6% | 30.3% | 7.6% | 85.8% | 8.5% | 70.1% | 34.1% | 19.5% | 92.6% | 11.8% | 60.2% | 24.4% | 14.2% | 92.3% |
| 881 | 42.7% | 63.8% | 32.3% | 23.4% | 74.7% | 21.1% | 47.6% | 23.6% | 20.2% | 85.3% | 32.0% | 49.9% | 25.1% | 17.6% | 86.8% |
| 882 | 22.0% | 69.7% | 39.1% | 25.1% | 75.7% | 6.3% | 63.0% | 20.7% | 15.1% | 83.8% | 14.8% | 64.2% | 23.6% | 14.7% | 86.6% |
| 883 | 23.5% | 65.5% | 45.8% | 17.3% | 75.1% | 6.7% | 32.9% | 20.9% | 15.7% | 81.9% | 15.7% | 44.4% | 24.3% | 14.0% | 84.5% |
| 884 | 18.8% | 79.0% | 29.0% | 33.8% | 88.3% | 3.8% | 33.8% | 11.8% | 17.1% | 89.9% | 10.0% | 41.0% | 14.1% | 15.3% | 91.8% |
| 885 | 21.8% | 69.1% | 37.5% | 19.1% | 76.8% | 7.5% | 53.3% | 25.1% | 17.4% | 85.4% | 14.0% | 54.6% | 25.0% | 16.4% | 87.3% |
| 886 | 39.5% | 63.8% | 35.9% | 32.6% | 77.3% | 21.6% | 42.6% | 24.7% | 16.1% | 86.3% | 31.0% | 46.9% | 21.9% | 15.3% | 86.6% |
| 887 | 36.0% | 71.7% | 21.8% | 20.9% | 78.1% | 6.8% | 72.5% | 20.7% | 11.9% | 87.8% | 21.3% | 71.6% | 19.6% | 12.5% | 88.5% |
| 888 | 19.9% | 58.0% | 38.8% | 14.4% | 81.8% | 5.6% | 28.8% | 20.9% | 12.0% | 87.0% | 15.2% | 41.6% | 22.2% | 11.4% | 88.4% |
| 889 | 17.5% | 55.8% | 26.5% | 30.7% | 73.3% | 5.8% | 45.4% | 20.4% | 19.8% | 83.7% | 17.0% | 50.4% | 22.3% | 18.8% | 84.1% |
| 890 | 22.6% | 65.0% | 31.1% | 22.3% | 75.9% | 7.0% | 47.0% | 21.2% | 16.7% | 85.3% | 16.2% | 53.7% | 24.2% | 14.5% | 86.5% |
| 891 | 48.7% | 66.0% | 27.0% | 23.6% | 76.6% | 23.3% | 41.3% | 21.8% | 18.0% | 87.9% | 43.5% | 51.7% | 22.4% | 15.6% | 89.2% |
| 892 | 39.1% | 77.4% | 22.3% | 22.7% | 82.2% | 15.8% | 75.8% | 22.5% | 33.0% | 89.9% | 21.2% | 78.9% | 21.0% | 20.7% | 90.0% |
| 893 | 24.6% | 50.6% | 28.3% | 12.6% | 75.0% | 7.2% | 40.7% | 30.3% | 23.2% | 88.8% | 20.0% | 43.3% | 23.4% | 14.2% | 86.6% |
| 894 | 17.9% | 69.3% | 27.5% | 38.5% | 81.7% | 5.6% | 41.6% | 17.2% | 21.2% | 88.1% | 14.6% | 49.1% | 17.4% | 17.8% | 89.2% |
| 895 | 26.0% | 58.0% | 21.2% | 20.2% | 78.6% | 9.6% | 74.7% | 47.8% | 24.0% | 90.3% | 19.0% | 60.9% | 29.5% | 17.5% | 90.6% |
| 896 | 49.6% | 63.0% | 25.9% | 19.6% | 72.8% | 24.3% | 46.7% | 20.5% | 14.6% | 85.2% | 41.8% | 55.8% | 26.1% | 15.6% | 87.6% |
| 897 | 32.9% | 62.4% | 22.2% | 18.7% | 71.8% | 25.8% | 73.2% | 34.5% | 29.9% | 88.8% | 29.6% | 73.4% | 26.3% | 17.5% | 88.0% |
| 898 | 27.7% | 57.6% | 29.9% | 14.0% | 74.4% | 9.4% | 44.3% | 29.5% | 16.7% | 84.7% | 21.6% | 53.6% | 30.4% | 15.1% | 86.7% |
| 899 | 25.4% | 56.1% | 23.5% | 28.0% | 72.7% | 6.6% | 55.7% | 19.1% | 17.2% | 86.4% | 22.3% | 55.2% | 23.9% | 19.4% | 86.5% |
| 900 | 30.9% | 52.7% | 24.0% | 19.1% | 70.6% | 9.6% | 70.1% | 54.7% | 36.9% | 87.8% | 23.4% | 63.7% | 34.9% | 22.6% | 87.3% |
| 901 | 42.7% | 58.7% | 26.1% | 16.5% | 70.3% | 32.7% | 58.1% | 28.6% | 16.4% | 85.8% | 35.5% | 59.0% | 26.8% | 18.1% | 86.2% |
| 902 | 34.4% | 62.0% | 23.4% | 19.3% | 70.0% | 10.5% | 66.3% | 23.7% | 16.0% | 85.0% | 25.8% | 67.6% | 24.3% | 15.7% | 86.0% |
| 903 | 28.9% | 60.2% | 31.4% | 13.6% | 74.0% | 7.3% | 54.4% | 36.3% | 21.0% | 86.5% | 25.1% | 56.5% | 31.2% | 15.9% | 87.5% |
| 904 | 30.1% | 60.5% | 24.2% | 21.7% | 72.3% | 6.6% | 58.2% | 20.3% | 19.0% | 85.9% | 21.4% | 60.2% | 23.2% | 17.2% | 86.5% |
| 905 | 30.2% | 56.7% | 24.9% | 15.7% | 68.5% | 8.5% | 61.8% | 33.9% | 19.7% | 86.0% | 22.9% | 60.8% | 28.1% | 18.4% | 86.7% |
| 906 | 56.2% | 68.8% | 29.4% | 14.8% | 76.3% | 38.3% | 69.4% | 51.6% | 22.7% | 87.7% | 49.3% | 69.4% | 34.2% | 16.4% | 88.6% |
| 907 | 37.0% | 63.2% | 31.5% | 20.6% | 70.5% | 13.5% | 64.2% | 23.8% | 15.5% | 84.2% | 27.3% | 68.2% | 27.1% | 18.9% | 86.0% |
| 908 | 29.0% | 59.4% | 31.5% | 13.9% | 72.0% | 9.2% | 53.1% | 33.6% | 18.5% | 84.3% | 25.7% | 57.6% | 29.7% | 15.7% | 86.1% |
| 909 | 28.0% | 64.3% | 26.3% | 21.1% | 72.0% | 8.5% | 60.7% | 24.8% | 19.7% | 85.3% | 21.1% | 65.3% | 27.1% | 19.8% | 86.6% |
| 910 | 35.3% | 65.2% | 24.7% | 15.9% | 74.4% | 13.4% | 70.8% | 55.7% | 23.9% | 87.9% | 28.1% | 72.9% | 42.2% | 18.2% | 88.4% |
| 911 | 44.7% | 62.6% | 27.5% | 16.0% | 71.0% | 30.3% | 60.6% | 35.3% | 30.4% | 85.1% | 41.7% | 65.1% | 30.5% | 20.1% | 86.5% |
| 912 | 35.6% | 60.2% | 40.3% | 23.3% | 66.9% | 8.6% | 60.9% | 26.8% | 18.5% | 82.7% | 24.6% | 66.9% | 32.4% | 19.9% | 84.5% |
| 913 | 34.0% | 64.7% | 35.8% | 14.4% | 73.2% | 8.0% | 55.2% | 40.3% | 21.8% | 84.3% | 25.0% | 63.4% | 33.6% | 17.3% | 85.2% |
| 914 | 25.4% | 60.2% | 26.5% | 20.8% | 67.9% | 5.7% | 55.5% | 22.2% | 21.4% | 81.0% | 20.6% | 65.9% | 27.8% | 21.5% | 84.7% |
| 915 | 32.1% | 61.7% | 34.5% | 16.9% | 69.1% | 7.4% | 59.2% | 37.0% | 19.9% | 83.5% | 24.0% | 67.8% | 35.2% | 19.8% | 86.8% |
| 916 | 44.8% | 62.9% | 40.0% | 16.3% | 68.9% | 20.5% | 63.3% | 49.8% | 35.5% | 84.3% | 40.3% | 66.0% | 42.0% | 21.7% | 84.3% |
| 917 | 32.9% | 60.0% | 40.6% | 22.3% | 67.5% | 9.5% | 62.4% | 36.4% | 31.0% | 84.5% | 24.6% | 65.7% | 37.5% | 22.3% | 84.6% |
| 918 | 23.2% | 59.3% | 33.0% | 21.1% | 67.2% | 6.7% | 65.2% | 47.2% | 40.3% | 85.7% | 22.7% | 66.1% | 36.7% | 27.9% | 84.9% |
| 919 | 26.1% | 58.5% | 38.4% | 16.4% | 66.9% | 8.3% | 59.1% | 38.0% | 22.7% | 81.6% | 22.7% | 64.0% | 36.1% | 18.3% | 83.0% |
| 920 | 40.8% | 63.8% | 48.0% | 32.7% | 71.3% | 10.0% | 64.7% | 32.4% | 24.5% | 85.2% | 27.5% | 70.0% | 43.3% | 28.9% | 86.6% |
| 921 | 37.9% | 67.7% | 50.5% | 17.6% | 74.2% | 7.0% | 55.6% | 36.7% | 24.1% | 86.5% | 30.8% | 65.7% | 41.6% | 18.0% | 86.3% |
| 922 | 25.5% | 62.8% | 41.7% | 27.9% | 70.0% | 5.8% | 55.0% | 26.8% | 28.4% | 85.2% | 21.3% | 68.2% | 38.0% | 29.1% | 86.8% |
| 923 | 29.6% | 61.5% | 46.2% | 20.3% | 69.4% | 8.0% | 62.1% | 38.9% | 23.3% | 83.9% | 22.7% | 65.6% | 39.7% | 18.9% | 84.4% |
| 924 | 41.9% | 61.3% | 44.5% | 25.8% | 68.0% | 24.6% | 58.1% | 33.3% | 23.5% | 86.4% | 37.0% | 66.8% | 44.0% | 26.4% | 84.6% |
| 925 | 37.6% | 61.5% | 46.7% | 37.4% | 67.5% | 6.6% | 65.2% | 38.2% | 35.3% | 85.0% | 30.1% | 68.1% | 40.9% | 29.0% | 84.7% |
| 926 | 28.7% | 59.6% | 43.2% | 26.1% | 68.7% | 6.4% | 51.2% | 35.2% | 34.2% | 84.0% | 25.4% | 63.8% | 39.8% | 24.1% | 86.6% |
| 927 | 24.3% | 61.1% | 40.4% | 29.8% | 68.7% | 6.3% | 66.3% | 34.3% | 31.4% | 86.0% | 22.6% | 67.4% | 39.8% | 31.3% | 84.9% |
| 928 | 28.0% | 58.4% | 37.5% | 21.5% | 68.5% | 24.1% | 57.3% | 34.1% | 15.9% | 82.0% | 28.7% | 62.4% | 36.7% | 20.1% | 83.0% |
| 929 | 41.4% | 62.2% | 47.4% | 31.1% | 69.8% | 38.5% | 64.9% | 40.2% | 23.0% | 87.2% | 42.8% | 70.0% | 45.8% | 27.8% | 87.0% |
| 930 | 47.4% | 71.6% | 60.1% | 51.8% | 76.5% | 8.3% | 69.0% | 23.9% | 17.3% | 87.6% | 29.9% | 73.6% | 41.5% | 29.8% | 88.4% |
| 931 | 31.5% | 58.3% | 41.3% | 28.7% | 67.5% | 6.7% | 40.7% | 28.7% | 24.0% | 83.3% | 24.9% | 66.9% | 40.0% | 23.5% | 86.6% |
| 932 | 28.3% | 61.9% | 44.9% | 35.9% | 68.3% | 5.6% | 54.5% | 24.8% | 27.8% | 86.3% | 25.0% | 68.1% | 36.2% | 31.9% | 85.7% |
| 933 | 29.3% | 62.9% | 46.1% | 25.2% | 70.4% | 9.5% | 61.0% | 29.3% | 20.3% | 86.2% | 25.9% | 70.0% | 39.1% | 22.0% | 87.2% |
| 934 | 61.7% | 75.6% | 63.1% | 43.4% | 80.1% | 26.0% | 58.1% | 34.5% | 25.9% | 90.7% | 55.7% | 77.2% | 50.2% | 27.1% | 90.8% |
| 935 | 41.7% | 66.4% | 53.0% | 46.6% | 72.7% | 9.3% | 77.6% | 26.7% | 20.2% | 90.3% | 20.0% | 72.9% | 49.0% | 38.2% | 87.6% |
| 936 | 31.8% | 60.9% | 46.9% | 29.5% | 68.7% | 5.5% | 51.3% | 38.9% | 27.9% | 84.9% | 22.0% | 65.5% | 41.0% | 19.4% | 84.5% |
| 937 | 32.6% | 61.2% | 43.0% | 38.9% | 68.5% | 5.2% | 66.2% | 45.0% | 42.0% | 87.3% | 25.6% | 66.0% | 38.7% | 31.7% | 84.6% |
| 938 | 32.4% | 64.1% | 45.3% | 31.8% | 72.2% | 10.6% | 65.6% | 37.4% | 28.8% | 88.7% | 24.3% | 66.8% | 40.3% | 25.9% | 88.0% |
| 939 | 41.9% | 60.7% | 42.8% | 32.6% | 67.8% | 21.6% | 59.8% | 29.7% | 44.6% | 86.8% | 44.5% | 68.9% | 38.7% | 31.7% | 86.0% |
| 940 | 37.4% | 62.9% | 46.1% | 40.7% | 69.5% | 10.8% | 69.4% | 31.7% | 31.1% | 86.1% | 30.5% | 70.6% | 42.7% | 33.9% | 88.1% |
| 941 | 29.1% | 60.4% | 42.4% | 35.3% | 68.1% | 6.2% | 50.3% | 25.0% | 28.1% | 85.9% | 21.3% | 66.4% | 31.0% | 25.3% | 85.5% |
| 942 | 31.3% | 63.1% | 46.3% | 31.4% | 70.0% | 5.5% | 68.8% | 45.4% | 36.6% | 87.7% | 22.6% | 66.4% | 43.4% | 26.4% | 84.5% |
| 943 | 48.7% | 66.0% | 50.6% | 33.9% | 72.4% | 32.7% | 60.6% | 35.7% | 28.5% | 90.9% | 47.8% | 74.5% | 49.8% | 25.8% | 89.6% |
| 944 | 39.3% | 70.2% | 57.9% | 51.7% | 75.3% | 7.2% | 74.2% | 29.5% | 24.0% | 90.1% | 19.2% | 72.0% | 49.8% | 38.8% | 87.4% |
| 945 | 26.2% | 58.4% | 42.1% | 25.5% | 66.2% | 6.7% | 42.6% | 29.9% | 22.4% | 84.6% | 18.1% | 63.9% | 38.9% | 20.0% | 85.1% |
| 946 | 23.8% | 58.8% | 42.0% | 35.1% | 66.7% | 5.2% | 47.0% | 20.4% | 23.6% | 86.0% | 15.6% | 62.9% | 32.0% | 26.1% | 84.1% |
| 947 | 27.1% | 64.0% | 47.8% | 31.8% | 70.8% | 7.3% | 59.9% | 30.6% | 23.6% | 87.5% | 18.8% | 68.3% | 41.8% | 22.5% | 85.9% |
| 948 | 47.6% | 68.6% | 54.5% | 36.4% | 74.3% | 24.3% | 39.9% | 21.6% | 17.1% | 87.3% | 33.3% | 69.2% | 46.1% | 25.1% | 86.8% |
| 949 | 29.8% | 62.8% | 48.1% | 40.2% | 70.1% | 8.8% | 69.2% | 42.1% | 44.0% | 86.4% | 18.4% | 71.7% | 45.7% | 34.6% | 87.6% |
| 950 | 21.5% | 62.4% | 48.3% | 30.4% | 69.8% | 5.9% | 37.2% | 27.5% | 19.9% | 85.8% | 17.8% | 64.0% | 42.3% | 20.9% | 85.8% |
| 951 | 27.4% | 62.0% | 45.0% | 38.4% | 69.1% | 4.8% | 50.5% | 24.8% | 26.6% | 83.8% | 16.6% | 66.1% | 35.7% | 27.4% | 83.9% |
| 952 | 25.0% | 66.0% | 47.7% | 27.9% | 72.8% | 5.4% | 64.5% | 41.3% | 35.3% | 88.0% | 16.4% | 71.7% | 42.9% | 22.4% | 88.6% |
| 953 | 43.9% | 64.9% | 50.0% | 31.4% | 71.6% | 30.0% | 45.0% | 22.7% | 33.8% | 90.5% | 37.2% | 69.0% | 46.9% | 24.3% | 86.1% |
| 954 | 31.6% | 66.0% | 51.9% | 43.5% | 72.2% | 5.9% | 69.1% | 22.3% | 18.0% | 87.6% | 15.8% | 72.0% | 44.7% | 34.0% | 87.8% |
| 955 | 22.1% | 61.0% | 44.1% | 27.4% | 70.6% | 13.6% | 45.2% | 27.0% | 16.0% | 88.4% | 18.6% | 65.9% | 41.7% | 19.3% | 88.2% |
| 956 | 22.2% | 61.3% | 43.2% | 35.0% | 69.0% | 5.4% | 43.4% | 19.4% | 22.2% | 86.0% | 13.3% | 66.2% | 35.3% | 27.9% | 85.0% |
| 957 | 21.7% | 67.0% | 51.8% | 31.5% | 73.3% | 7.6% | 58.6% | 28.6% | 22.7% | 89.0% | 15.4% | 70.8% | 45.1% | 23.8% | 87.6% |
| 958 | 30.3% | 62.5% | 46.8% | 40.5% | 69.1% | 5.9% | 67.3% | 29.2% | 21.3% | 85.5% | 18.9% | 71.8% | 43.0% | 32.9% | 87.7% |
| 959 | 23.0% | 61.7% | 46.3% | 31.4% | 69.5% | 6.3% | 34.9% | 23.6% | 17.0% | 85.5% | 16.7% | 65.6% | 41.7% | 19.4% | 88.0% |
| 960 | 20.9% | 61.1% | 43.8% | 34.4% | 68.9% | 6.5% | 46.1% | 19.2% | 22.6% | 85.5% | 14.6% | 68.2% | 39.8% | 35.3% | 86.6% |
| 961 | 22.3% | 67.4% | 52.3% | 33.9% | 73.8% | 4.3% | 55.0% | 29.9% | 30.7% | 89.8% | 13.6% | 77.1% | 51.0% | 31.1% | 91.0% |
| 962 | 40.9% | 57.8% | 43.7% | 27.8% | 65.2% | 18.7% | 42.2% | 22.4% | 18.0% | 84.4% | 35.8% | 66.4% | 39.5% | 20.9% | 85.7% |
| 963 | 31.8% | 64.0% | 49.5% | 41.1% | 70.8% | 7.7% | 68.4% | 22.4% | 16.9% | 87.0% | 18.4% | 73.4% | 43.1% | 28.3% | 88.3% |
| 964 | 24.7% | 61.3% | 45.3% | 29.5% | 69.4% | 6.1% | 36.7% | 23.8% | 18.6% | 84.7% | 17.2% | 62.4% | 40.1% | 17.3% | 88.0% |
| 965 | 22.2% | 62.1% | 43.2% | 35.5% | 69.6% | 5.5% | 48.0% | 24.0% | 32.4% | 85.4% | 14.7% | 68.7% | 36.5% | 33.8% | 87.0% |
| 966 | 23.8% | 61.0% | 45.8% | 30.6% | 67.9% | 5.6% | 51.4% | 24.9% | 18.9% | 85.2% | 16.6% | 66.6% | 38.6% | 25.6% | 86.5% |
| 967 | 59.2% | 73.7% | 61.1% | 35.0% | 78.5% | 22.3% | 45.5% | 29.0% | 27.4% | 89.9% | 53.2% | 73.8% | 47.2% | 25.4% | 91.6% |
| 968 | 32.6% | 63.9% | 47.1% | 35.9% | 71.0% | 8.5% | 68.6% | 25.9% | 25.2% | 87.0% | 19.2% | 73.0% | 30.2% | 23.2% | 87.7% |
| 969 | 25.2% | 62.2% | 45.9% | 26.8% | 68.9% | 7.4% | 36.8% | 24.8% | 17.2% | 84.8% | 17.9% | 59.9% | 37.5% | 18.3% | 88.2% |
| 970 | 21.4% | 59.4% | 38.6% | 31.5% | 67.4% | 4.7% | 46.4% | 23.5% | 27.0% | 85.8% | 17.0% | 61.5% | 34.0% | 36.3% | 87.3% |
| 971 | 47.8% | 65.7% | 44.9% | 29.1% | 72.0% | 29.4% | 51.6% | 24.3% | 48.8% | 89.0% | 51.5% | 68.3% | 31.6% | 39.9% | 91.5% |
| 972 | 43.6% | 67.3% | 51.4% | 40.8% | 73.1% | 12.8% | 71.4% | 31.6% | 23.4% | 87.8% | 26.6% | 74.5% | 29.8% | 22.3% | 89.5% |
| 973 | 25.2% | 59.2% | 43.6% | 24.4% | 68.3% | 5.9% | 50.9% | 37.2% | 29.3% | 85.9% | 20.4% | 59.5% | 37.8% | 22.5% | 86.2% |
| 974 | 23.4% | 65.7% | 39.8% | 32.8% | 72.9% | 3.2% | 39.0% | 15.7% | 26.3% | 88.1% | 16.9% | 64.7% | 24.6% | 29.6% | 90.7% |
| 975 | 28.5% | 67.5% | 41.3% | 24.8% | 73.3% | 6.0% | 68.6% | 29.3% | 45.2% | 90.0% | 22.6% | 71.1% | 31.0% | 38.6% | 91.1% |
| 976 | 50.2% | 65.5% | 40.0% | 23.9% | 71.3% | 60.4% | 66.5% | 36.1% | 14.6% | 89.0% | 58.7% | 71.8% | 37.1% | 19.9% | 89.4% |
| 977 | 54.3% | 76.7% | 54.5% | 44.6% | 80.9% | 5.2% | 71.1% | 19.4% | 15.4% | 87.8% | 25.1% | 81.0% | 22.2% | 16.4% | 91.2% |
| 978 | 32.8% | 66.3% | 48.7% | 20.1% | 73.4% | 4.4% | 30.8% | 21.9% | 13.2% | 86.2% | 21.9% | 53.3% | 30.3% | 13.2% | 89.8% |
| 979 | 28.0% | 64.0% | 35.2% | 30.2% | 71.1% | 5.2% | 52.5% | 33.6% | 36.9% | 86.1% | 23.1% | 65.2% | 31.4% | 33.1% | 88.3% |
| 980 | 32.0% | 66.8% | 42.0% | 24.6% | 72.8% | 42.1% | 76.9% | 35.5% | 13.8% | 91.4% | 38.9% | 78.4% | 33.9% | 16.9% | 91.9% |
| 981 | 39.9% | 62.8% | 40.4% | 31.2% | 69.3% | 7.3% | 68.3% | 26.0% | 20.5% | 86.7% | 26.2% | 68.1% | 29.1% | 19.9% | 85.7% |
| 982 | 29.7% | 60.3% | 41.5% | 17.4% | 68.1% | 5.2% | 36.8% | 26.5% | 18.5% | 86.1% | 23.2% | 62.6% | 33.2% | 14.2% | 86.6% |
| 983 | 28.4% | 64.9% | 36.8% | 29.9% | 71.5% | 3.4% | 50.6% | 25.1% | 27.8% | 87.4% | 23.6% | 69.2% | 29.5% | 26.8% | 89.9% |
| 984 | 30.9% | 62.5% | 37.6% | 21.0% | 69.9% | 5.5% | 58.0% | 32.5% | 28.3% | 86.9% | 21.6% | 64.8% | 33.3% | 23.2% | 87.9% |
| 985 | 51.1% | 66.8% | 45.2% | 25.6% | 72.3% | 29.7% | 45.2% | 25.9% | 18.1% | 87.3% | 52.8% | 69.7% | 34.2% | 20.1% | 88.2% |
| 986 | 46.4% | 68.9% | 45.7% | 37.5% | 74.9% | 7.3% | 71.0% | 28.6% | 26.9% | 87.4% | 29.9% | 76.8% | 32.3% | 25.1% | 89.5% |
| 987 | 34.1% | 63.8% | 46.0% | 21.3% | 70.6% | 5.6% | 35.1% | 25.1% | 16.6% | 84.5% | 23.9% | 58.9% | 33.3% | 14.8% | 86.1% |
| 988 | 31.3% | 67.0% | 39.6% | 33.4% | 73.1% | 5.3% | 45.8% | 20.2% | 24.4% | 88.7% | 25.4% | 69.9% | 25.8% | 25.1% | 89.2% |
| 989 | 37.3% | 66.9% | 44.2% | 24.5% | 72.8% | 5.0% | 51.0% | 25.4% | 17.6% | 85.2% | 25.0% | 64.9% | 30.1% | 18.5% | 88.8% |
| 990 | 42.0% | 60.9% | 33.3% | 24.4% | 70.8% | 21.3% | 47.2% | 27.3% | 20.8% | 88.2% | 47.2% | 66.3% | 32.2% | 23.8% | 90.3% |
| 991 | 36.7% | 65.7% | 38.3% | 31.9% | 72.6% | 8.5% | 66.6% | 22.8% | 14.9% | 86.3% | 24.8% | 75.0% | 30.7% | 23.6% | 90.3% |
| 992 | 28.2% | 60.1% | 38.3% | 19.1% | 69.5% | 7.5% | 44.4% | 34.3% | 21.6% | 87.2% | 22.6% | 62.9% | 37.0% | 19.6% | 88.6% |
| 993 | 25.5% | 62.9% | 36.0% | 32.9% | 71.7% | 5.8% | 53.1% | 24.9% | 30.7% | 87.7% | 18.8% | 67.3% | 31.5% | 31.8% | 89.1% |
| 994 | 29.4% | 62.5% | 35.6% | 24.5% | 72.0% | 7.9% | 56.9% | 29.2% | 24.6% | 88.2% | 23.8% | 67.3% | 32.8% | 25.1% | 90.4% |
| 995 | 40.2% | 67.3% | 29.8% | 11.2% | 75.9% | 15.5% | 28.9% | 15.4% | 14.8% | 86.1% | 23.2% | 43.1% | 20.2% | 15.1% | 88.2% |
| 996 | 37.0% | 75.2% | 21.0% | 14.6% | 81.5% | 5.2% | 56.2% | 15.8% | 11.8% | 88.0% | 15.5% | 63.1% | 19.6% | 11.9% | 90.3% |
| 997 | 14.8% | 68.9% | 23.0% | 13.9% | 76.5% | 6.5% | 50.5% | 19.1% | 18.5% | 85.6% | 13.7% | 60.8% | 22.9% | 17.6% | 88.4% |
| 998 | 17.7% | 66.7% | 33.9% | 13.6% | 75.6% | 8.2% | 40.6% | 20.1% | 16.7% | 87.3% | 15.6% | 52.5% | 26.6% | 16.8% | 88.4% |
| 999 | 27.2% | 62.5% | 31.4% | 19.5% | 75.5% | 14.7% | 36.9% | 18.1% | 14.8% | 86.2% | 21.4% | 47.6% | 24.7% | 17.5% | 87.7% |
| 1000 | 29.1% | 70.0% | 36.0% | 26.1% | 76.5% | 12.0% | 65.3% | 24.3% | 17.0% | 85.6% | 18.5% | 71.0% | 25.5% | 16.6% | 88.8% |
| 1001 | 23.8% | 64.1% | 45.7% | 19.6% | 73.8% | 6.9% | 38.4% | 27.0% | 18.3% | 83.2% | 18.8% | 51.9% | 29.9% | 18.0% | 87.7% |
| 1002 | 25.6% | 80.9% | 37.4% | 39.2% | 87.1% | 4.7% | 43.1% | 19.4% | 29.9% | 89.9% | 13.7% | 53.3% | 20.4% | 28.7% | 92.1% |
| 1003 | 19.1% | 67.7% | 45.5% | 17.3% | 76.6% | 6.2% | 44.9% | 20.8% | 15.6% | 86.4% | 14.8% | 53.9% | 26.3% | 16.3% | 87.3% |
| 1004 | 26.0% | 68.2% | 34.7% | 34.0% | 75.1% | 19.9% | 68.8% | 30.3% | 23.0% | 86.5% | 19.8% | 67.6% | 26.1% | 18.7% | 87.3% |
| 1005 | 22.4% | 63.1% | 46.6% | 26.2% | 77.9% | 6.9% | 35.2% | 23.9% | 14.2% | 85.6% | 17.3% | 48.6% | 27.3% | 15.3% | 86.9% |
| 1006 | 15.4% | 62.5% | 41.9% | 43.2% | 73.8% | 6.5% | 43.0% | 19.4% | 18.7% | 84.9% | 14.7% | 53.5% | 25.5% | 23.4% | 86.3% |
| 1007 | 23.1% | 60.2% | 35.6% | 30.6% | 74.0% | 6.9% | 42.8% | 21.9% | 30.7% | 85.0% | 16.2% | 49.0% | 26.4% | 22.5% | 86.8% |
| 1008 | 51.4% | 62.4% | 27.9% | 26.6% | 76.6% | 26.9% | 40.5% | 20.6% | 15.3% | 86.3% | 41.3% | 51.5% | 23.5% | 16.5% | 89.2% |
| 1009 | 26.8% | 57.7% | 24.0% | 21.8% | 67.6% | 8.6% | 61.9% | 20.6% | 16.3% | 84.5% | 19.6% | 65.0% | 25.0% | 17.3% | 86.2% |
| 1010 | 25.4% | 62.4% | 38.3% | 17.1% | 77.6% | 7.3% | 37.2% | 31.0% | 21.4% | 86.4% | 18.2% | 50.2% | 29.3% | 15.4% | 87.7% |
| 1011 | 19.5% | 63.0% | 27.2% | 37.9% | 75.8% | 5.1% | 38.8% | 16.9% | 22.5% | 86.0% | 16.2% | 49.7% | 21.6% | 21.1% | 87.5% |
| 1012 | 26.7% | 60.1% | 25.0% | 25.6% | 81.0% | 17.3% | 64.0% | 31.0% | 16.2% | 89.8% | 18.2% | 57.8% | 25.1% | 14.7% | 90.4% |
| 1013 | 41.3% | 58.2% | 25.6% | 20.3% | 71.6% | 29.9% | 57.1% | 31.1% | 19.1% | 85.9% | 36.2% | 56.9% | 27.6% | 18.2% | 86.8% |
| 1014 | 38.4% | 65.7% | 26.5% | 26.7% | 73.8% | 9.4% | 65.7% | 23.4% | 16.3% | 85.5% | 20.4% | 68.5% | 25.3% | 17.0% | 87.1% |
| 1015 | 38.2% | 61.4% | 30.6% | 13.2% | 78.2% | 11.5% | 36.6% | 25.8% | 14.2% | 88.1% | 23.5% | 49.8% | 27.6% | 13.4% | 88.1% |
| 1016 | 34.1% | 65.2% | 22.6% | 26.5% | 76.2% | 8.7% | 60.4% | 28.6% | 29.7% | 88.1% | 21.2% | 64.0% | 24.7% | 23.1% | 89.1% |
| 1017 | 26.5% | 57.6% | 24.9% | 18.2% | 69.7% | 11.9% | 55.6% | 25.1% | 17.8% | 85.5% | 19.5% | 58.3% | 26.4% | 18.3% | 86.7% |
| 1018 | 36.1% | 60.4% | 27.6% | 20.6% | 68.1% | 9.7% | 61.3% | 26.0% | 16.7% | 83.7% | 25.8% | 66.5% | 27.5% | 19.3% | 86.0% |
| 1019 | 33.8% | 56.5% | 29.0% | 14.0% | 72.8% | 17.5% | 56.2% | 36.5% | 23.9% | 87.0% | 29.2% | 57.0% | 30.6% | 16.9% | 87.3% |
| 1020 | 35.2% | 61.7% | 23.7% | 19.5% | 72.0% | 7.9% | 51.3% | 18.9% | 20.5% | 86.3% | 22.8% | 60.9% | 25.3% | 19.9% | 85.8% |
| 1021 | 35.2% | 52.6% | 24.7% | 16.3% | 68.0% | 11.4% | 57.9% | 30.6% | 35.0% | 85.4% | 22.2% | 53.1% | 28.4% | 22.9% | 85.9% |
| 1022 | 39.6% | 60.5% | 27.5% | 19.5% | 69.6% | 32.1% | 60.4% | 35.5% | 23.1% | 86.9% | 36.5% | 66.6% | 34.8% | 21.7% | 87.5% |
| 1023 | 37.1% | 60.3% | 28.4% | 19.3% | 68.5% | 21.3% | 62.9% | 31.7% | 29.0% | 84.4% | 31.9% | 69.7% | 32.4% | 20.6% | 86.8% |
| 1024 | 27.8% | 54.2% | 29.7% | 14.5% | 67.5% | 14.8% | 49.0% | 28.1% | 17.2% | 81.1% | 25.3% | 55.9% | 30.8% | 16.4% | 83.2% |
| 1025 | 32.3% | 59.6% | 26.3% | 21.0% | 69.4% | 6.5% | 57.0% | 21.4% | 20.0% | 81.8% | 23.2% | 61.6% | 25.9% | 20.9% | 85.6% |
| 1026 | 31.7% | 60.3% | 30.8% | 16.6% | 69.0% | 10.9% | 64.5% | 35.2% | 18.2% | 84.0% | 26.0% | 66.9% | 30.1% | 17.2% | 86.4% |
| 1027 | 38.4% | 60.9% | 32.5% | 19.0% | 69.2% | 26.2% | 54.3% | 28.2% | 21.9% | 84.9% | 33.4% | 64.6% | 31.9% | 20.9% | 88.2% |
| 1028 | 37.5% | 61.0% | 34.7% | 21.2% | 68.5% | 8.9% | 57.0% | 25.6% | 17.7% | 81.9% | 25.4% | 65.6% | 30.9% | 20.2% | 86.5% |
| 1029 | 38.9% | 64.2% | 37.0% | 15.3% | 73.7% | 7.8% | 53.2% | 38.9% | 22.6% | 87.1% | 28.3% | 64.0% | 36.3% | 18.0% | 87.1% |
| 1030 | 32.8% | 57.7% | 23.7% | 18.4% | 69.6% | 6.9% | 60.4% | 25.3% | 22.3% | 85.1% | 21.9% | 63.2% | 27.2% | 21.8% | 88.4% |
| 1031 | 32.6% | 58.8% | 33.3% | 17.1% | 66.7% | 8.6% | 57.2% | 35.0% | 21.0% | 83.0% | 22.6% | 65.2% | 32.2% | 18.6% | 85.2% |
| 1032 | 41.4% | 60.5% | 38.5% | 17.7% | 68.8% | 28.7% | 58.6% | 37.0% | 23.7% | 84.6% | 37.2% | 65.8% | 38.5% | 20.8% | 86.2% |
| 1033 | 38.5% | 63.5% | 45.0% | 25.4% | 71.1% | 8.5% | 62.0% | 34.6% | 30.3% | 84.1% | 26.1% | 69.4% | 41.3% | 25.8% | 87.7% |
| 1034 | 27.1% | 59.6% | 26.5% | 18.6% | 67.3% | 7.8% | 58.6% | 29.1% | 26.3% | 82.4% | 22.5% | 65.2% | 30.5% | 21.2% | 85.3% |
| 1035 | 30.5% | 57.5% | 37.0% | 17.7% | 66.3% | 6.3% | 50.2% | 28.1% | 32.5% | 82.8% | 16.6% | 61.9% | 34.1% | 23.4% | 85.3% |
| 1036 | 38.7% | 58.3% | 40.3% | 19.6% | 66.2% | 16.4% | 53.3% | 32.2% | 24.3% | 83.3% | 32.3% | 64.2% | 37.9% | 21.4% | 84.4% |
| 1037 | 41.0% | 63.4% | 47.1% | 36.5% | 69.4% | 7.9% | 59.9% | 26.3% | 19.7% | 82.6% | 20.3% | 66.3% | 34.5% | 23.0% | 83.5% |
| 1038 | 29.1% | 57.1% | 38.1% | 17.3% | 65.0% | 11.6% | 59.4% | 38.0% | 27.2% | 83.3% | 23.2% | 59.7% | 37.2% | 20.2% | 82.0% |
| 1039 | 31.9% | 64.7% | 35.0% | 24.1% | 71.5% | 5.5% | 55.9% | 34.3% | 35.3% | 85.0% | 21.1% | 66.7% | 34.8% | 28.5% | 86.0% |
| 1040 | 33.7% | 61.6% | 45.3% | 21.7% | 68.6% | 8.8% | 58.1% | 35.9% | 24.9% | 83.6% | 23.2% | 67.2% | 39.9% | 21.8% | 85.6% |
| 1041 | 42.0% | 62.7% | 42.2% | 23.1% | 70.6% | 19.1% | 56.4% | 32.9% | 25.7% | 84.8% | 36.9% | 67.8% | 43.0% | 26.2% | 88.1% |
| 1042 | 39.5% | 63.0% | 47.2% | 32.8% | 70.4% | 21.6% | 64.5% | 31.0% | 22.4% | 85.7% | 29.6% | 67.8% | 41.4% | 29.8% | 86.3% |
| 1043 | 29.7% | 58.9% | 43.3% | 25.6% | 67.5% | 6.7% | 50.5% | 35.0% | 30.3% | 84.3% | 22.6% | 66.2% | 42.5% | 23.2% | 86.2% |
| 1044 | 29.6% | 62.0% | 42.7% | 32.8% | 69.7% | 6.5% | 55.1% | 30.1% | 30.0% | 86.2% | 24.5% | 68.5% | 40.2% | 32.0% | 87.0% |
| 1045 | 31.0% | 60.2% | 44.8% | 25.6% | 68.8% | 13.0% | 61.2% | 35.6% | 26.8% | 85.5% | 23.2% | 66.7% | 43.3% | 27.7% | 85.6% |
| 1046 | 39.4% | 58.7% | 46.0% | 33.5% | 66.7% | 16.6% | 43.4% | 23.5% | 24.1% | 82.3% | 30.8% | 62.6% | 38.0% | 27.0% | 84.2% |
| 1047 | 37.6% | 60.1% | 44.5% | 37.1% | 66.7% | 8.6% | 62.7% | 27.6% | 24.4% | 85.3% | 27.3% | 64.9% | 40.6% | 33.0% | 85.1% |
| 1048 | 33.0% | 62.3% | 46.2% | 30.9% | 70.2% | 9.9% | 47.1% | 37.0% | 29.6% | 84.7% | 25.8% | 64.9% | 42.9% | 28.6% | 87.2% |
| 1049 | 34.3% | 69.3% | 50.1% | 42.8% | 75.9% | 8.8% | 54.0% | 36.3% | 43.0% | 86.6% | 24.3% | 68.2% | 42.2% | 39.0% | 87.4% |
| 1050 | 44.2% | 65.1% | 48.1% | 35.9% | 72.3% | 27.0% | 56.6% | 26.6% | 27.9% | 87.4% | 36.0% | 68.5% | 41.8% | 30.3% | 88.0% |
| 1051 | 43.9% | 69.1% | 56.4% | 50.5% | 74.9% | 6.9% | 55.5% | 19.0% | 23.9% | 87.0% | 17.7% | 72.0% | 36.2% | 35.3% | 87.1% |
| 1052 | 38.5% | 64.7% | 48.2% | 32.9% | 71.6% | 12.2% | 58.2% | 41.0% | 38.3% | 87.0% | 34.5% | 72.1% | 46.9% | 31.5% | 88.3% |
| 1053 | 32.7% | 61.4% | 45.1% | 36.8% | 69.7% | 5.8% | 44.6% | 24.2% | 28.2% | 85.6% | 20.9% | 65.6% | 38.9% | 33.7% | 86.4% |
| 1054 | 52.6% | 78.8% | 66.9% | 52.8% | 83.7% | 5.7% | 67.7% | 39.6% | 32.7% | 92.1% | 31.0% | 77.4% | 51.3% | 32.9% | 92.2% |
| 1055 | 48.0% | 67.5% | 53.2% | 39.0% | 73.1% | 27.3% | 58.6% | 35.6% | 37.4% | 88.6% | 40.6% | 73.5% | 51.6% | 33.9% | 87.6% |
| 1056 | 37.3% | 62.9% | 47.9% | 41.0% | 70.0% | 7.9% | 66.3% | 27.5% | 21.3% | 87.2% | 22.6% | 69.6% | 43.3% | 36.2% | 88.3% |
| 1057 | 30.9% | 62.8% | 46.9% | 33.4% | 71.0% | 7.9% | 41.2% | 28.4% | 24.8% | 87.4% | 23.0% | 66.5% | 40.8% | 22.6% | 86.9% |
| 1058 | 34.8% | 67.0% | 53.1% | 45.6% | 73.9% | 6.0% | 51.0% | 23.3% | 28.4% | 88.5% | 20.1% | 71.3% | 41.4% | 31.2% | 88.3% |
| 1059 | 40.2% | 69.1% | 55.8% | 42.4% | 75.4% | 4.6% | 55.6% | 32.5% | 33.2% | 87.2% | 23.8% | 69.9% | 47.1% | 30.0% | 86.8% |
| 1060 | 35.0% | 57.3% | 41.5% | 28.2% | 66.1% | 16.5% | 40.6% | 22.8% | 24.3% | 83.8% | 26.9% | 61.2% | 34.0% | 25.0% | 83.8% |
| 1061 | 32.3% | 62.0% | 45.8% | 37.5% | 69.0% | 7.4% | 64.8% | 33.2% | 26.1% | 86.0% | 20.5% | 67.3% | 42.9% | 33.7% | 87.3% |
| 1062 | 27.1% | 61.7% | 44.5% | 31.1% | 70.6% | 6.6% | 38.2% | 27.9% | 22.0% | 87.4% | 19.3% | 66.7% | 42.2% | 21.6% | 87.6% |
| 1063 | 25.8% | 64.7% | 46.2% | 40.6% | 73.2% | 5.3% | 43.7% | 21.2% | 26.6% | 87.5% | 17.9% | 68.4% | 35.9% | 31.2% | 87.8% |
| 1064 | 22.4% | 60.4% | 41.1% | 28.5% | 67.4% | 9.7% | 58.8% | 31.8% | 24.5% | 85.4% | 16.4% | 64.9% | 40.4% | 26.5% | 84.7% |
| 1065 | 36.9% | 60.4% | 45.9% | 31.9% | 68.1% | 24.1% | 41.0% | 22.3% | 18.2% | 85.5% | 30.4% | 64.7% | 41.0% | 21.4% | 84.0% |
| 1066 | 45.2% | 74.9% | 61.8% | 55.1% | 79.6% | 10.3% | 78.0% | 39.5% | 37.8% | 91.6% | 18.5% | 80.2% | 55.1% | 49.2% | 91.7% |
| 1067 | 25.0% | 62.8% | 47.1% | 32.2% | 71.3% | 7.1% | 39.5% | 26.3% | 20.3% | 85.1% | 19.5% | 65.1% | 42.5% | 25.1% | 86.6% |
| 1068 | 23.3% | 63.3% | 45.8% | 37.2% | 70.0% | 4.3% | 49.1% | 23.7% | 30.7% | 88.4% | 16.2% | 69.8% | 38.9% | 29.2% | 87.6% |
| 1069 | 26.6% | 68.8% | 53.2% | 35.6% | 75.7% | 9.1% | 64.8% | 26.8% | 26.9% | 89.8% | 15.5% | 71.9% | 42.2% | 34.9% | 88.9% |
| 1070 | 35.4% | 63.3% | 48.8% | 34.0% | 69.2% | 28.3% | 46.1% | 24.9% | 21.9% | 85.4% | 31.2% | 67.3% | 44.8% | 26.7% | 85.5% |
| 1071 | 29.6% | 66.4% | 52.2% | 44.4% | 73.5% | 13.1% | 75.0% | 30.5% | 30.0% | 90.7% | 21.3% | 70.6% | 46.5% | 38.8% | 88.4% |
| 1072 | 20.3% | 56.9% | 40.9% | 27.7% | 66.0% | 6.3% | 36.6% | 24.2% | 21.9% | 82.6% | 15.2% | 59.9% | 36.3% | 21.6% | 83.7% |
| 1073 | 20.1% | 59.3% | 40.9% | 34.4% | 67.0% | 6.1% | 45.8% | 22.3% | 24.4% | 85.8% | 15.0% | 67.7% | 40.1% | 31.0% | 87.4% |
| 1074 | 21.1% | 60.3% | 44.4% | 29.6% | 67.9% | 8.5% | 61.2% | 36.3% | 27.3% | 86.5% | 16.8% | 67.6% | 43.7% | 27.6% | 86.6% |
| 1075 | 37.6% | 64.0% | 47.1% | 32.0% | 71.3% | 22.8% | 44.1% | 24.0% | 21.1% | 86.3% | 35.6% | 69.7% | 43.1% | 26.2% | 89.0% |
| 1076 | 33.8% | 65.2% | 51.3% | 44.5% | 71.7% | 6.4% | 52.1% | 16.0% | 22.1% | 85.5% | 15.8% | 70.2% | 31.9% | 30.1% | 88.3% |
| 1077 | 20.3% | 60.4% | 45.6% | 31.9% | 69.5% | 7.9% | 41.3% | 29.1% | 26.8% | 83.7% | 18.4% | 61.4% | 40.6% | 26.4% | 84.9% |
| 1078 | 22.4% | 59.4% | 40.6% | 33.8% | 68.0% | 6.1% | 45.0% | 20.7% | 31.4% | 86.2% | 18.1% | 66.1% | 33.8% | 33.3% | 87.3% |
| 1079 | 30.7% | 66.4% | 48.2% | 42.1% | 73.8% | 8.6% | 50.7% | 21.3% | 24.3% | 83.8% | 17.4% | 64.1% | 28.2% | 26.4% | 86.1% |
| 1080 | 29.8% | 63.1% | 47.5% | 33.8% | 71.2% | 7.0% | 46.2% | 32.9% | 33.8% | 87.9% | 24.2% | 68.5% | 48.9% | 33.3% | 90.0% |
| 1081 | 22.5% | 62.5% | 44.5% | 38.0% | 70.2% | 8.9% | 43.2% | 34.0% | 42.5% | 88.3% | 19.8% | 67.2% | 44.3% | 43.0% | 88.6% |
| 1082 | 22.6% | 61.7% | 45.4% | 33.7% | 69.2% | 5.6% | 68.0% | 47.8% | 39.2% | 87.3% | 15.0% | 72.1% | 50.3% | 35.8% | 88.7% |
| 1083 | 41.0% | 62.2% | 46.6% | 34.5% | 69.7% | 16.2% | 35.5% | 20.2% | 24.2% | 87.0% | 38.3% | 62.4% | 33.8% | 29.3% | 90.3% |
| 1084 | 33.6% | 59.9% | 45.5% | 37.6% | 66.6% | 5.6% | 63.3% | 25.6% | 23.6% | 84.0% | 17.7% | 70.3% | 31.6% | 26.5% | 85.6% |
| 1085 | 24.3% | 58.5% | 42.9% | 28.5% | 66.8% | 6.8% | 41.3% | 30.7% | 23.3% | 85.1% | 17.9% | 61.6% | 41.6% | 23.9% | 88.8% |
| 1086 | 26.1% | 63.6% | 41.9% | 36.1% | 70.9% | 4.7% | 40.5% | 26.8% | 41.8% | 88.3% | 17.8% | 64.0% | 38.9% | 46.6% | 90.2% |
| 1087 | 25.9% | 64.7% | 47.7% | 32.2% | 71.0% | 7.0% | 52.4% | 26.3% | 20.4% | 86.3% | 17.5% | 64.5% | 35.8% | 29.4% | 89.2% |
| 1088 | 41.2% | 62.0% | 44.8% | 30.3% | 69.9% | 31.7% | 50.4% | 26.5% | 20.1% | 87.5% | 46.3% | 66.5% | 36.8% | 26.2% | 89.7% |
| 1089 | 44.5% | 66.3% | 49.7% | 43.9% | 72.0% | 6.5% | 70.7% | 19.0% | 13.2% | 87.7% | 25.0% | 78.1% | 22.5% | 18.4% | 90.3% |
| 1090 | 25.1% | 58.4% | 41.4% | 24.8% | 67.7% | 5.8% | 37.8% | 33.5% | 28.6% | 86.9% | 19.8% | 59.9% | 42.2% | 26.4% | 89.0% |
| 1091 | 25.4% | 63.6% | 41.0% | 33.9% | 71.7% | 5.7% | 40.5% | 19.7% | 28.7% | 84.9% | 18.6% | 59.9% | 25.4% | 30.4% | 88.9% |
| 1092 | 29.7% | 61.0% | 41.6% | 27.1% | 69.6% | 5.9% | 44.8% | 24.7% | 22.2% | 85.0% | 20.6% | 63.0% | 35.0% | 26.9% | 88.2% |
| 1093 | 47.0% | 64.3% | 47.1% | 29.4% | 71.2% | 34.4% | 49.1% | 26.3% | 22.1% | 92.0% | 61.4% | 70.9% | 38.8% | 25.4% | 92.8% |
| 1094 | 44.1% | 66.2% | 45.6% | 38.5% | 71.4% | 7.4% | 77.7% | 31.4% | 21.5% | 91.9% | 25.7% | 79.1% | 32.1% | 23.5% | 90.7% |
| 1095 | 33.6% | 64.3% | 46.2% | 22.3% | 72.3% | 5.1% | 33.0% | 27.8% | 21.1% | 89.9% | 27.1% | 65.1% | 39.7% | 17.9% | 90.7% |
| 1096 | 31.6% | 64.0% | 40.8% | 35.1% | 70.0% | 5.2% | 42.2% | 21.2% | 30.5% | 86.0% | 19.7% | 64.0% | 27.3% | 33.3% | 89.7% |
| 1097 | 33.6% | 64.1% | 41.0% | 24.6% | 71.0% | 8.4% | 58.8% | 33.4% | 38.0% | 89.2% | 25.7% | 70.3% | 37.8% | 34.7% | 91.9% |
| 1098 | 50.5% | 68.0% | 45.9% | 26.5% | 74.6% | 37.2% | 44.8% | 25.8% | 20.8% | 88.3% | 53.0% | 68.1% | 34.1% | 22.3% | 90.4% |
| 1099 | 46.1% | 67.3% | 45.3% | 38.6% | 73.3% | 10.7% | 75.6% | 36.7% | 29.3% | 89.7% | 27.1% | 78.2% | 35.1% | 25.1% | 90.2% |
| 1100 | 34.1% | 63.1% | 43.9% | 21.0% | 70.8% | 24.5% | 65.5% | 57.2% | 42.4% | 89.9% | 28.8% | 68.1% | 46.7% | 24.8% | 89.2% |
| 1101 | 32.5% | 65.0% | 40.1% | 34.7% | 72.3% | 4.7% | 63.1% | 25.1% | 30.0% | 90.2% | 21.5% | 74.1% | 33.6% | 34.1% | 90.5% |
| 1102 | 38.1% | 66.1% | 41.1% | 24.1% | 72.4% | 6.2% | 53.1% | 35.9% | 38.8% | 90.4% | 18.9% | 63.9% | 35.6% | 31.7% | 87.7% |
| 1103 | 52.4% | 68.4% | 44.2% | 28.1% | 75.2% | 43.7% | 55.0% | 28.5% | 25.0% | 89.0% | 56.5% | 69.4% | 33.7% | 25.4% | 91.8% |
| 1104 | 56.0% | 72.7% | 51.2% | 44.7% | 77.3% | 6.9% | 65.8% | 22.5% | 15.9% | 84.4% | 24.2% | 75.0% | 26.4% | 19.1% | 88.0% |
| 1105 | 35.6% | 66.7% | 41.5% | 37.5% | 72.6% | 5.4% | 45.5% | 21.3% | 25.6% | 86.8% | 21.8% | 68.5% | 29.7% | 29.8% | 89.1% |
| 1106 | 39.6% | 66.7% | 43.7% | 28.5% | 72.7% | 6.1% | 50.7% | 26.7% | 22.5% | 87.5% | 22.5% | 64.9% | 32.4% | 22.1% | 88.8% |
| 1107 | 41.0% | 61.0% | 34.1% | 24.1% | 70.5% | 15.3% | 42.6% | 24.1% | 17.7% | 87.3% | 40.1% | 65.7% | 32.3% | 22.2% | 89.9% |
| 1108 | 29.1% | 63.9% | 36.0% | 29.4% | 71.0% | 9.5% | 74.5% | 60.2% | 41.9% | 90.0% | 16.6% | 75.0% | 51.9% | 31.6% | 89.9% |
| 1109 | 26.6% | 60.3% | 37.5% | 20.4% | 69.8% | 6.4% | 37.5% | 25.4% | 17.1% | 87.5% | 20.6% | 64.7% | 34.5% | 18.3% | 88.7% |
| 1110 | 20.4% | 61.8% | 34.0% | 31.5% | 70.0% | 5.2% | 47.3% | 18.9% | 24.1% | 87.0% | 17.8% | 66.9% | 29.7% | 30.4% | 89.9% |
| 1111 | 23.5% | 61.9% | 34.2% | 24.5% | 70.2% | 6.4% | 48.7% | 25.3% | 19.1% | 87.6% | 19.6% | 64.2% | 29.6% | 22.5% | 88.9% |
| 1112 | 36.7% | 65.0% | 39.2% | 28.3% | 76.8% | 11.9% | 35.3% | 18.8% | 15.1% | 86.0% | 23.5% | 49.4% | 25.8% | 18.2% | 88.7% |
| 1113 | 29.0% | 65.3% | 29.8% | 25.1% | 72.7% | 6.9% | 51.1% | 20.4% | 14.6% | 83.9% | 17.9% | 64.4% | 24.5% | 16.0% | 87.1% |
| 1114 | 12.8% | 62.5% | 43.7% | 12.9% | 71.1% | 5.5% | 30.1% | 18.3% | 13.2% | 84.3% | 13.6% | 45.6% | 26.4% | 13.4% | 84.4% |
| 1115 | 13.8% | 69.4% | 31.9% | 25.5% | 75.5% | 6.2% | 36.2% | 16.2% | 17.6% | 84.6% | 12.3% | 54.9% | 23.8% | 21.1% | 86.9% |
| 1116 | 19.2% | 77.3% | 46.3% | 17.1% | 84.0% | 4.8% | 41.4% | 16.4% | 12.8% | 88.7% | 12.6% | 50.8% | 21.6% | 13.5% | 89.7% |
| 1117 | 56.7% | 68.5% | 30.0% | 29.3% | 82.7% | 11.8% | 29.0% | 14.8% | 12.6% | 89.6% | 36.6% | 46.3% | 19.2% | 14.2% | 90.8% |
| 1118 | 25.3% | 65.1% | 22.4% | 23.1% | 73.1% | 6.5% | 62.0% | 15.1% | 11.6% | 85.6% | 16.4% | 59.5% | 21.8% | 15.0% | 86.2% |
| 1119 | 17.0% | 57.0% | 39.3% | 19.8% | 72.8% | 6.3% | 35.0% | 20.4% | 13.6% | 85.9% | 18.0% | 50.5% | 27.4% | 16.8% | 88.3% |
| 1120 | 17.8% | 60.0% | 29.9% | 32.6% | 72.8% | 7.1% | 44.1% | 19.0% | 19.2% | 85.7% | 15.9% | 55.2% | 25.1% | 21.7% | 87.1% |
| 1121 | 18.4% | 74.0% | 44.1% | 34.8% | 81.9% | 5.0% | 36.3% | 16.5% | 15.8% | 89.2% | 13.0% | 45.5% | 22.5% | 18.0% | 89.3% |
| 1122 | 47.5% | 61.0% | 28.2% | 25.7% | 72.9% | 14.4% | 35.7% | 17.2% | 16.9% | 86.7% | 34.8% | 52.3% | 25.1% | 18.5% | 89.4% |
| 1123 | 38.8% | 72.6% | 18.8% | 18.5% | 79.6% | 5.9% | 67.3% | 14.3% | 10.3% | 87.3% | 19.3% | 68.1% | 19.1% | 12.1% | 89.6% |
| 1124 | 23.6% | 58.9% | 29.6% | 14.0% | 71.4% | 7.0% | 32.3% | 19.4% | 18.8% | 87.5% | 18.9% | 50.3% | 26.1% | 15.6% | 89.0% |
| 1125 | 22.3% | 64.6% | 29.0% | 40.5% | 79.1% | 5.4% | 34.0% | 15.6% | 17.8% | 88.0% | 16.7% | 47.5% | 20.9% | 18.8% | 87.9% |
| 1126 | 24.1% | 72.2% | 24.7% | 24.0% | 83.3% | 6.3% | 40.8% | 13.8% | 13.2% | 89.3% | 15.8% | 50.3% | 18.1% | 12.0% | 90.9% |
| 1127 | 44.6% | 57.3% | 26.3% | 20.9% | 71.9% | 21.7% | 45.3% | 20.7% | 15.4% | 84.8% | 33.9% | 54.3% | 25.9% | 16.0% | 85.8% |
| 1128 | 42.0% | 70.9% | 26.8% | 25.8% | 77.9% | 11.3% | 67.5% | 18.2% | 15.7% | 87.0% | 25.9% | 73.2% | 22.7% | 16.8% | 90.4% |
| 1129 | 30.1% | 54.7% | 29.9% | 16.2% | 70.8% | 7.8% | 35.7% | 23.1% | 16.3% | 86.5% | 19.6% | 51.9% | 28.4% | 17.3% | 86.6% |
| 1130 | 22.2% | 60.1% | 27.3% | 29.5% | 70.9% | 6.8% | 44.5% | 18.8% | 20.4% | 85.3% | 19.2% | 57.7% | 24.5% | 21.1% | 86.9% |
| 1131 | 34.5% | 59.8% | 25.6% | 19.4% | 74.2% | 8.7% | 55.8% | 19.4% | 14.7% | 87.6% | 17.5% | 53.8% | 22.0% | 15.6% | 88.6% |
| 1132 | 47.0% | 60.7% | 26.4% | 19.9% | 73.3% | 18.4% | 42.6% | 18.5% | 16.4% | 88.0% | 36.9% | 58.1% | 25.5% | 18.0% | 88.8% |
| 1133 | 37.8% | 63.0% | 27.3% | 20.0% | 70.0% | 9.9% | 63.6% | 21.5% | 15.0% | 85.5% | 25.3% | 67.8% | 26.2% | 18.7% | 86.8% |
| 1134 | 34.3% | 57.2% | 29.6% | 15.4% | 73.4% | 11.5% | 34.0% | 18.2% | 13.4% | 84.1% | 21.5% | 51.1% | 25.9% | 15.0% | 86.9% |
| 1135 | 26.4% | 56.4% | 24.1% | 20.3% | 67.7% | 7.1% | 47.9% | 20.6% | 23.0% | 85.2% | 19.5% | 56.5% | 24.6% | 18.6% | 84.2% |
| 1136 | 30.8% | 54.4% | 23.9% | 16.1% | 70.5% | 8.3% | 53.0% | 21.5% | 14.8% | 85.9% | 23.1% | 56.2% | 25.7% | 16.7% | 87.5% |
| 1137 | 38.2% | 60.6% | 30.9% | 18.6% | 69.5% | 23.6% | 52.7% | 29.2% | 19.3% | 82.1% | 35.9% | 62.5% | 28.2% | 18.7% | 87.3% |
| 1138 | 36.0% | 62.0% | 31.8% | 23.9% | 68.9% | 9.9% | 59.6% | 25.0% | 17.6% | 83.6% | 21.6% | 68.6% | 29.0% | 19.8% | 86.7% |
| 1139 | 25.7% | 53.8% | 29.4% | 14.0% | 66.4% | 10.6% | 51.7% | 27.2% | 18.5% | 84.7% | 21.6% | 56.7% | 28.9% | 16.5% | 84.8% |
| 1140 | 29.9% | 62.9% | 25.7% | 19.1% | 70.4% | 6.2% | 49.8% | 19.1% | 20.6% | 84.6% | 18.8% | 64.6% | 26.2% | 20.4% | 86.9% |
| 1141 | 23.8% | 58.5% | 27.6% | 15.2% | 67.1% | 10.8% | 52.1% | 19.8% | 14.0% | 84.0% | 20.0% | 60.3% | 25.5% | 16.9% | 84.8% |
| 1142 | 42.8% | 63.0% | 42.0% | 19.3% | 70.5% | 16.6% | 44.1% | 22.2% | 16.7% | 85.1% | 34.0% | 64.5% | 31.9% | 19.2% | 86.5% |
| 1143 | 39.2% | 66.2% | 35.6% | 22.7% | 72.6% | 10.1% | 62.3% | 23.9% | 16.5% | 85.2% | 24.4% | 69.4% | 29.6% | 19.3% | 88.1% |
| 1144 | 26.1% | 57.6% | 35.2% | 16.3% | 66.2% | 8.2% | 47.3% | 29.4% | 18.9% | 81.8% | 21.5% | 59.9% | 32.6% | 17.5% | 84.7% |
| 1145 | 26.9% | 59.6% | 29.3% | 21.0% | 68.1% | 6.3% | 50.6% | 23.0% | 22.8% | 84.7% | 19.6% | 63.6% | 29.9% | 22.0% | 85.4% |
| 1146 | 28.0% | 61.1% | 31.3% | 14.8% | 68.3% | 9.9% | 62.8% | 33.9% | 19.2% | 85.8% | 20.0% | 63.7% | 28.6% | 17.2% | 85.7% |
| 1147 | 43.2% | 62.3% | 39.9% | 18.7% | 70.2% | 18.7% | 44.6% | 22.4% | 15.6% | 84.1% | 35.7% | 62.6% | 31.6% | 17.8% | 86.1% |
| 1148 | 30.0% | 57.6% | 34.9% | 21.2% | 65.5% | 7.6% | 55.9% | 22.9% | 18.2% | 82.7% | 23.8% | 64.4% | 31.0% | 20.2% | 85.9% |
| 1149 | 28.3% | 59.6% | 39.1% | 16.5% | 69.3% | 7.6% | 47.6% | 31.1% | 23.0% | 82.9% | 21.5% | 64.2% | 40.0% | 18.5% | 86.9% |
| 1150 | 25.0% | 57.3% | 31.7% | 21.4% | 64.7% | 6.5% | 47.7% | 23.3% | 26.0% | 81.5% | 17.6% | 60.8% | 29.1% | 21.9% | 81.0% |
| 1151 | 28.3% | 59.9% | 42.2% | 18.6% | 68.5% | 5.9% | 57.0% | 30.7% | 27.7% | 84.9% | 19.0% | 66.7% | 36.5% | 20.5% | 87.8% |
| 1152 | 35.4% | 60.8% | 41.2% | 19.6% | 68.7% | 15.0% | 46.3% | 23.4% | 17.8% | 84.1% | 31.2% | 66.5% | 35.1% | 19.4% | 87.2% |
| 1153 | 35.9% | 60.4% | 44.9% | 29.0% | 67.8% | 7.7% | 59.8% | 26.2% | 17.9% | 83.0% | 25.0% | 66.3% | 36.1% | 22.7% | 85.5% |
| 1154 | 26.4% | 58.5% | 41.4% | 24.9% | 67.3% | 6.3% | 39.2% | 25.4% | 26.8% | 84.6% | 18.4% | 65.8% | 42.0% | 24.3% | 86.2% |
| 1155 | 21.3% | 57.7% | 38.3% | 26.2% | 66.1% | 6.8% | 48.3% | 24.0% | 26.1% | 83.4% | 17.4% | 62.4% | 37.4% | 27.5% | 84.0% |
| 1156 | 26.2% | 59.9% | 37.5% | 17.4% | 68.1% | 8.8% | 60.0% | 30.7% | 21.8% | 85.8% | 21.7% | 67.1% | 35.8% | 20.1% | 86.3% |
| 1157 | 38.9% | 60.6% | 42.0% | 22.7% | 68.7% | 19.5% | 50.8% | 27.7% | 24.8% | 81.7% | 30.6% | 64.4% | 38.5% | 22.7% | 83.1% |
| 1158 | 35.3% | 61.4% | 45.6% | 31.1% | 68.7% | 6.7% | 60.7% | 30.3% | 25.8% | 83.7% | 21.0% | 67.5% | 41.7% | 29.3% | 85.5% |
| 1159 | 25.2% | 58.6% | 39.9% | 24.4% | 67.4% | 6.6% | 46.1% | 30.3% | 26.9% | 84.7% | 22.3% | 65.6% | 41.6% | 23.4% | 86.5% |
| 1160 | 27.2% | 57.3% | 40.9% | 31.7% | 65.9% | 6.5% | 45.2% | 22.8% | 24.4% | 84.2% | 17.9% | 65.6% | 39.8% | 31.2% | 85.4% |
| 1161 | 28.4% | 60.2% | 43.3% | 28.2% | 67.5% | 6.1% | 61.7% | 36.6% | 31.6% | 86.3% | 18.4% | 66.7% | 42.3% | 27.6% | 85.9% |
| 1162 | 37.7% | 61.7% | 45.1% | 30.2% | 69.4% | 12.7% | 40.1% | 22.1% | 26.2% | 85.6% | 28.4% | 63.9% | 38.4% | 26.5% | 88.2% |
| 1163 | 39.3% | 66.6% | 53.8% | 47.5% | 72.7% | 7.9% | 67.0% | 22.4% | 19.1% | 85.9% | 27.5% | 68.5% | 42.4% | 34.8% | 86.1% |
| 1164 | 27.0% | 59.4% | 42.0% | 27.1% | 68.2% | 7.6% | 42.9% | 26.3% | 24.0% | 85.0% | 21.2% | 64.2% | 38.2% | 21.0% | 86.6% |
| 1165 | 22.1% | 56.8% | 39.6% | 31.4% | 65.9% | 6.3% | 45.3% | 25.7% | 26.5% | 84.3% | 16.9% | 61.2% | 34.8% | 29.2% | 83.8% |
| 1166 | 31.9% | 67.5% | 51.4% | 36.9% | 73.6% | 7.0% | 61.9% | 30.7% | 25.4% | 87.7% | 20.8% | 69.3% | 46.1% | 30.4% | 87.2% |
| 1167 | 37.8% | 61.2% | 44.6% | 34.1% | 69.2% | 13.5% | 41.0% | 22.0% | 21.1% | 86.4% | 30.6% | 68.1% | 42.7% | 29.5% | 88.0% |
| 1168 | 48.3% | 70.2% | 57.8% | 53.4% | 76.3% | 6.1% | 72.5% | 26.6% | 22.3% | 88.7% | 26.0% | 72.7% | 51.2% | 42.9% | 87.7% |
| 1169 | 28.1% | 60.8% | 45.4% | 33.6% | 68.6% | 6.4% | 43.2% | 28.1% | 23.9% | 86.5% | 20.3% | 66.2% | 43.5% | 23.6% | 85.9% |
| 1170 | 33.1% | 64.8% | 48.8% | 42.7% | 71.6% | 4.8% | 44.0% | 20.1% | 25.2% | 88.1% | 21.2% | 69.0% | 41.2% | 34.8% | 87.3% |
| 1171 | 33.0% | 63.6% | 47.4% | 33.3% | 70.3% | 8.8% | 66.0% | 35.6% | 24.9% | 86.4% | 24.6% | 69.1% | 41.2% | 24.3% | 86.9% |
| 1172 | 38.1% | 63.1% | 47.1% | 37.0% | 70.4% | 13.5% | 42.2% | 22.3% | 19.4% | 86.2% | 30.6% | 68.0% | 41.9% | 28.8% | 87.1% |
| 1173 | 34.6% | 61.1% | 45.7% | 38.4% | 68.8% | 7.9% | 64.1% | 23.4% | 17.7% | 86.9% | 23.1% | 70.2% | 43.8% | 35.9% | 88.6% |
| 1174 | 22.0% | 55.7% | 38.8% | 27.9% | 65.5% | 6.1% | 37.5% | 24.5% | 20.1% | 85.1% | 20.6% | 62.2% | 35.6% | 20.5% | 85.1% |
| 1175 | 24.6% | 61.7% | 43.9% | 36.3% | 68.6% | 5.1% | 38.1% | 21.3% | 25.8% | 86.8% | 15.9% | 64.8% | 37.4% | 31.5% | 84.9% |
| 1176 | 22.7% | 59.7% | 44.9% | 33.8% | 68.5% | 5.9% | 54.5% | 25.9% | 22.3% | 85.4% | 16.7% | 63.2% | 37.7% | 26.4% | 85.8% |
| 1177 | 34.3% | 60.6% | 43.5% | 31.2% | 68.8% | 12.5% | 41.1% | 21.4% | 19.9% | 85.6% | 23.9% | 66.1% | 42.9% | 27.5% | 87.7% |
| 1178 | 29.9% | 58.5% | 41.9% | 35.5% | 66.3% | 12.0% | 60.4% | 25.2% | 19.6% | 84.3% | 20.8% | 65.5% | 40.7% | 31.0% | 85.4% |
| 1179 | 23.5% | 61.2% | 44.9% | 28.9% | 69.2% | 5.6% | 41.6% | 28.5% | 26.0% | 87.2% | 16.5% | 68.1% | 44.6% | 22.6% | 86.3% |
| 1180 | 20.0% | 59.1% | 41.9% | 34.2% | 67.1% | 5.3% | 43.6% | 19.2% | 23.9% | 83.8% | 14.4% | 63.5% | 34.1% | 26.3% | 82.9% |
| 1181 | 27.2% | 66.5% | 51.4% | 35.9% | 73.3% | 5.2% | 51.4% | 26.7% | 28.8% | 86.7% | 15.5% | 69.4% | 44.5% | 28.3% | 87.4% |
| 1182 | 36.1% | 61.3% | 45.8% | 32.4% | 68.6% | 19.2% | 41.4% | 21.8% | 17.1% | 84.8% | 26.2% | 66.2% | 41.2% | 21.7% | 86.1% |
| 1183 | 40.6% | 74.2% | 61.5% | 55.5% | 79.2% | 7.4% | 68.5% | 19.8% | 15.4% | 88.3% | 16.3% | 75.4% | 46.5% | 35.7% | 90.1% |
| 1184 | 21.2% | 67.3% | 52.8% | 33.9% | 73.8% | 6.1% | 35.3% | 23.4% | 17.3% | 85.2% | 14.0% | 68.5% | 43.6% | 21.8% | 86.6% |
| 1185 | 25.8% | 68.6% | 50.1% | 45.7% | 75.4% | 4.6% | 36.8% | 22.2% | 36.8% | 88.3% | 12.9% | 70.4% | 39.3% | 40.8% | 90.0% |
| 1186 | 32.9% | 61.6% | 46.0% | 31.1% | 69.3% | 15.2% | 42.2% | 20.4% | 20.8% | 86.2% | 23.6% | 67.2% | 42.2% | 22.6% | 86.7% |
| 1187 | 24.8% | 60.6% | 45.0% | 38.7% | 68.7% | 6.9% | 58.3% | 20.9% | 17.5% | 85.1% | 17.7% | 67.2% | 40.5% | 30.1% | 87.8% |
| 1188 | 19.0% | 61.3% | 43.5% | 29.5% | 69.2% | 6.6% | 52.8% | 38.3% | 26.9% | 87.4% | 15.2% | 65.6% | 44.2% | 26.2% | 87.1% |
| 1189 | 18.9% | 56.3% | 38.2% | 31.5% | 66.1% | 5.6% | 38.1% | 17.8% | 19.8% | 83.4% | 15.0% | 61.1% | 35.9% | 29.5% | 83.9% |
| 1190 | 20.8% | 64.9% | 49.5% | 33.8% | 71.8% | 6.0% | 48.0% | 22.5% | 19.6% | 86.7% | 14.9% | 67.8% | 43.3% | 24.1% | 86.8% |
| 1191 | 31.9% | 63.2% | 46.6% | 30.8% | 71.2% | 11.3% | 41.6% | 25.0% | 26.6% | 86.7% | 25.1% | 71.2% | 47.3% | 29.3% | 90.3% |
| 1192 | 31.9% | 64.1% | 49.1% | 40.9% | 70.8% | 7.1% | 65.5% | 29.6% | 17.4% | 85.7% | 16.9% | 74.7% | 44.1% | 29.0% | 89.4% |
| 1193 | 20.1% | 68.8% | 56.4% | 41.3% | 74.9% | 4.1% | 22.8% | 18.3% | 13.2% | 89.2% | 11.7% | 71.1% | 56.7% | 18.5% | 91.5% |
| 1194 | 18.6% | 60.3% | 40.5% | 32.3% | 68.5% | 5.8% | 35.1% | 17.8% | 21.1% | 84.4% | 13.7% | 64.2% | 33.1% | 27.3% | 86.9% |
| 1195 | 20.1% | 62.0% | 45.1% | 33.1% | 70.1% | 5.4% | 40.6% | 19.8% | 16.9% | 86.3% | 14.4% | 68.1% | 38.6% | 27.9% | 89.0% |
| 1196 | 40.9% | 70.9% | 54.0% | 40.4% | 77.3% | 16.6% | 35.5% | 18.9% | 27.3% | 91.5% | 33.8% | 70.1% | 45.0% | 35.4% | 93.4% |
| 1197 | 35.1% | 64.1% | 48.1% | 41.0% | 70.2% | 6.0% | 60.4% | 19.8% | 21.5% | 86.5% | 17.4% | 74.7% | 33.3% | 32.6% | 89.8% |
| 1198 | 17.6% | 63.0% | 47.3% | 30.3% | 70.6% | 5.1% | 29.5% | 23.2% | 19.8% | 86.5% | 13.0% | 65.9% | 45.1% | 19.0% | 88.7% |
| 1199 | 19.8% | 63.3% | 44.4% | 37.6% | 71.2% | 6.6% | 42.0% | 18.8% | 28.1% | 87.4% | 15.9% | 67.4% | 37.8% | 37.8% | 90.4% |
| 1200 | 18.1% | 63.6% | 47.4% | 30.7% | 71.2% | 5.0% | 51.5% | 26.5% | 25.1% | 87.8% | 13.7% | 71.0% | 43.5% | 27.8% | 88.9% |
| 1201 | 37.3% | 59.6% | 40.2% | 30.1% | 69.0% | 11.8% | 36.4% | 19.5% | 21.4% | 87.7% | 35.2% | 61.9% | 34.2% | 29.4% | 89.8% |
| 1202 | 30.5% | 60.5% | 44.0% | 36.8% | 68.3% | 6.0% | 56.9% | 19.6% | 16.7% | 83.3% | 18.2% | 69.2% | 27.0% | 24.7% | 87.9% |
| 1203 | 21.2% | 62.6% | 44.8% | 25.9% | 70.5% | 5.1% | 33.0% | 22.6% | 17.1% | 85.7% | 16.5% | 60.9% | 36.0% | 17.1% | 88.1% |
| 1204 | 20.0% | 61.0% | 39.5% | 32.5% | 68.5% | 5.3% | 38.5% | 18.1% | 24.7% | 84.8% | 14.3% | 60.9% | 27.6% | 31.1% | 87.8% |
| 1205 | 23.1% | 65.2% | 47.9% | 34.8% | 72.7% | 7.1% | 45.6% | 20.9% | 17.4% | 88.0% | 14.8% | 62.3% | 34.8% | 28.1% | 90.8% |
| 1206 | 43.2% | 61.8% | 43.8% | 32.0% | 68.7% | 13.8% | 34.1% | 16.7% | 16.2% | 86.9% | 42.1% | 59.6% | 27.4% | 22.3% | 87.5% |
| 1207 | 33.7% | 57.2% | 39.0% | 28.9% | 67.8% | 10.9% | 60.6% | 24.9% | 15.8% | 84.3% | 24.5% | 64.7% | 27.8% | 17.7% | 85.2% |
| 1208 | 24.2% | 61.8% | 42.2% | 25.5% | 69.8% | 4.6% | 29.0% | 20.4% | 31.1% | 89.0% | 17.0% | 59.1% | 37.9% | 27.6% | 89.9% |
| 1209 | 21.8% | 59.4% | 37.9% | 31.6% | 66.8% | 5.2% | 33.9% | 15.3% | 25.0% | 86.3% | 15.2% | 56.1% | 26.7% | 33.7% | 86.5% |
| 1210 | 22.8% | 62.6% | 43.5% | 27.7% | 69.6% | 6.2% | 43.9% | 19.4% | 15.1% | 88.2% | 14.9% | 60.2% | 26.2% | 19.9% | 89.6% |
| 1211 | 59.7% | 74.1% | 50.0% | 28.8% | 79.5% | 16.6% | 28.9% | 13.9% | 12.0% | 89.5% | 57.0% | 60.8% | 23.0% | 16.2% | 93.2% |
| 1212 | 48.3% | 73.8% | 56.4% | 46.2% | 79.0% | 4.7% | 70.1% | 15.5% | 16.7% | 90.4% | 25.9% | 80.0% | 23.0% | 19.3% | 92.3% |
| 1213 | 30.8% | 63.7% | 46.5% | 22.3% | 70.6% | 4.5% | 25.6% | 17.5% | 17.4% | 85.4% | 23.2% | 59.1% | 33.0% | 15.6% | 87.9% |
| 1214 | 22.3% | 65.8% | 40.7% | 34.9% | 72.7% | 4.7% | 39.0% | 18.8% | 24.1% | 85.4% | 14.6% | 59.2% | 24.5% | 29.0% | 88.5% |
| 1215 | 34.9% | 69.6% | 45.5% | 26.8% | 75.7% | 3.4% | 41.3% | 19.8% | 20.3% | 93.0% | 16.3% | 61.8% | 26.6% | 22.1% | 93.1% |
| 1216 | 46.2% | 65.2% | 41.9% | 27.1% | 72.7% | 15.1% | 36.4% | 19.1% | 16.7% | 88.5% | 46.6% | 65.6% | 29.8% | 19.7% | 89.6% |
| 1217 | 43.1% | 65.0% | 45.0% | 37.8% | 72.2% | 7.0% | 65.9% | 20.3% | 15.5% | 87.8% | 20.8% | 73.2% | 28.5% | 21.3% | 88.6% |
| 1218 | 29.7% | 61.0% | 42.0% | 19.9% | 69.1% | 6.1% | 40.4% | 28.7% | 31.4% | 87.7% | 26.9% | 66.5% | 35.3% | 23.5% | 88.3% |
| 1219 | 26.2% | 60.6% | 35.9% | 30.1% | 69.3% | 5.3% | 42.2% | 19.2% | 24.4% | 86.9% | 20.6% | 64.8% | 27.5% | 29.6% | 87.9% |
| 1220 | 33.9% | 63.1% | 40.7% | 25.4% | 70.9% | 5.7% | 43.5% | 19.9% | 18.6% | 85.9% | 20.7% | 61.1% | 28.4% | 20.6% | 86.0% |
| 1221 | 44.8% | 65.3% | 41.3% | 26.9% | 73.4% | 15.9% | 43.3% | 25.0% | 32.0% | 87.6% | 42.4% | 68.0% | 37.8% | 27.5% | 89.5% |
| 1222 | 40.1% | 67.6% | 47.2% | 37.9% | 74.3% | 7.9% | 71.2% | 55.8% | 33.8% | 88.5% | 21.0% | 73.9% | 51.7% | 29.5% | 89.9% |
| 1223 | 28.2% | 63.3% | 43.6% | 21.1% | 70.3% | 6.8% | 35.8% | 24.7% | 21.5% | 85.3% | 22.0% | 66.9% | 39.2% | 20.0% | 88.5% |
| 1224 | 26.3% | 62.7% | 37.2% | 32.5% | 70.6% | 4.9% | 47.4% | 21.0% | 40.0% | 88.5% | 17.7% | 68.5% | 27.2% | 35.3% | 90.9% |
| 1225 | 36.0% | 64.9% | 43.3% | 24.2% | 71.7% | 9.0% | 65.1% | 34.3% | 21.6% | 88.2% | 27.9% | 69.1% | 33.7% | 19.7% | 88.9% |
| 1226 | 38.9% | 61.5% | 33.9% | 25.6% | 71.0% | 15.2% | 37.4% | 19.4% | 15.8% | 86.2% | 33.4% | 61.6% | 28.7% | 21.0% | 88.3% |
| 1227 | 23.1% | 64.0% | 33.3% | 29.1% | 71.1% | 9.3% | 68.8% | 26.5% | 18.8% | 88.0% | 17.8% | 72.5% | 29.4% | 23.4% | 90.4% |
| 1228 | 22.5% | 59.8% | 36.8% | 19.7% | 70.8% | 7.0% | 45.3% | 34.6% | 22.8% | 87.5% | 18.4% | 63.3% | 39.2% | 18.6% | 88.6% |
| 1229 | 16.3% | 59.3% | 30.4% | 29.8% | 69.2% | 5.4% | 41.9% | 15.4% | 19.5% | 86.8% | 13.5% | 64.9% | 28.7% | 28.0% | 88.1% |
| 1230 | 15.3% | 62.7% | 34.8% | 24.5% | 72.3% | 5.6% | 53.9% | 24.3% | 18.0% | 88.0% | 12.6% | 61.0% | 27.2% | 21.2% | 89.2% |
| 1231 | 50.1% | 63.5% | 27.4% | 24.8% | 76.5% | 12.5% | 25.9% | 13.8% | 10.0% | 85.3% | 28.7% | 41.0% | 18.0% | 12.8% | 86.2% |
| 1232 | 26.9% | 68.6% | 24.9% | 26.9% | 76.6% | 6.6% | 60.1% | 15.6% | 11.4% | 85.8% | 16.6% | 67.3% | 21.2% | 15.8% | 89.0% |
| 1233 | 19.8% | 68.7% | 48.3% | 22.0% | 79.2% | 6.0% | 44.4% | 25.5% | 13.1% | 87.4% | 16.8% | 51.0% | 27.7% | 15.6% | 88.6% |
| 1234 | 12.2% | 66.0% | 37.0% | 38.8% | 76.4% | 5.9% | 62.3% | 24.2% | 21.6% | 88.0% | 11.2% | 65.3% | 23.6% | 21.2% | 89.5% |
| 1235 | 12.5% | 66.3% | 50.6% | 36.9% | 77.4% | 6.0% | 34.0% | 16.4% | 13.6% | 82.5% | 12.4% | 47.1% | 25.8% | 18.3% | 86.0% |
| 1236 | 54.0% | 66.1% | 25.3% | 25.9% | 78.7% | 17.2% | 29.1% | 13.0% | 10.5% | 87.2% | 37.9% | 47.0% | 19.4% | 14.4% | 90.1% |
| 1237 | 31.4% | 74.1% | 22.4% | 23.3% | 81.1% | 6.6% | 65.5% | 14.5% | 10.6% | 87.4% | 17.1% | 68.3% | 18.5% | 12.4% | 90.9% |
| 1238 | 16.0% | 59.0% | 33.8% | 17.6% | 72.1% | 7.1% | 33.5% | 18.1% | 14.6% | 83.8% | 14.6% | 51.1% | 25.1% | 15.5% | 87.1% |
| 1239 | 12.8% | 61.4% | 30.7% | 40.0% | 74.1% | 5.0% | 38.5% | 19.5% | 20.7% | 85.6% | 12.1% | 48.3% | 22.7% | 21.9% | 86.6% |
| 1240 | 41.7% | 61.6% | 26.9% | 24.1% | 73.2% | 17.7% | 32.5% | 15.7% | 12.1% | 85.6% | 27.0% | 48.7% | 21.4% | 14.1% | 84.3% |
| 1241 | 27.8% | 63.0% | 21.2% | 22.9% | 73.5% | 6.5% | 63.0% | 15.8% | 12.1% | 87.3% | 18.8% | 69.1% | 21.8% | 15.7% | 89.1% |
| 1242 | 24.5% | 48.9% | 26.4% | 14.7% | 69.4% | 9.8% | 46.5% | 28.8% | 21.8% | 84.4% | 19.5% | 51.3% | 28.2% | 16.4% | 87.5% |
| 1243 | 20.8% | 46.2% | 20.6% | 21.8% | 71.6% | 5.7% | 64.8% | 24.0% | 16.8% | 85.6% | 18.7% | 59.4% | 24.7% | 17.8% | 87.2% |
| 1244 | 36.1% | 55.3% | 23.7% | 18.6% | 68.5% | 6.8% | 38.3% | 19.1% | 14.5% | 82.2% | 22.7% | 50.6% | 23.3% | 16.6% | 84.5% |
| 1245 | 31.2% | 57.3% | 24.2% | 17.7% | 66.7% | 8.1% | 56.1% | 17.8% | 14.0% | 82.8% | 22.8% | 61.4% | 25.0% | 16.7% | 85.3% |
| 1246 | 33.2% | 59.6% | 29.9% | 14.8% | 72.1% | 6.9% | 34.6% | 21.3% | 14.8% | 84.5% | 20.3% | 53.7% | 27.3% | 15.6% | 86.8% |
| 1247 | 33.6% | 60.0% | 24.5% | 25.4% | 72.3% | 5.8% | 48.1% | 16.1% | 14.4% | 87.1% | 24.9% | 57.2% | 22.1% | 20.5% | 88.2% |
| 1248 | 33.3% | 59.1% | 25.3% | 15.2% | 68.8% | 9.6% | 57.0% | 29.7% | 17.3% | 82.4% | 22.4% | 60.8% | 26.6% | 16.9% | 85.0% |
| 1249 | 49.9% | 66.9% | 28.6% | 20.3% | 75.2% | 35.4% | 41.6% | 15.1% | 11.9% | 86.1% | 42.2% | 51.8% | 22.5% | 15.4% | 88.4% |
| 1250 | 35.6% | 63.2% | 31.7% | 21.0% | 70.1% | 12.4% | 62.4% | 22.5% | 17.7% | 85.2% | 23.5% | 67.0% | 24.5% | 18.0% | 86.0% |
| 1251 | 22.1% | 58.4% | 33.2% | 15.2% | 68.0% | 7.1% | 47.6% | 29.0% | 18.3% | 84.2% | 18.1% | 60.8% | 30.9% | 17.3% | 85.5% |
| 1252 | 19.1% | 66.2% | 29.6% | 30.4% | 74.1% | 8.4% | 56.2% | 16.9% | 19.1% | 85.2% | 14.6% | 60.7% | 23.2% | 21.9% | 88.1% |
| 1253 | 16.0% | 58.3% | 25.2% | 15.2% | 66.9% | 11.8% | 54.8% | 21.1% | 14.7% | 82.8% | 18.8% | 63.1% | 26.2% | 17.4% | 85.2% |
| 1254 | 37.8% | 58.9% | 32.8% | 17.5% | 67.3% | 16.1% | 47.0% | 23.0% | 15.9% | 83.7% | 35.3% | 63.6% | 29.1% | 18.2% | 85.7% |
| 1255 | 34.0% | 57.7% | 36.2% | 17.9% | 65.7% | 10.1% | 60.8% | 20.3% | 14.1% | 83.3% | 25.0% | 64.2% | 29.3% | 18.4% | 86.1% |
| 1256 | 14.7% | 60.0% | 32.7% | 14.5% | 68.6% | 5.9% | 56.2% | 36.2% | 21.7% | 84.6% | 15.3% | 62.7% | 32.4% | 16.4% | 86.0% |
| 1257 | 20.5% | 62.8% | 26.8% | 20.9% | 70.7% | 5.2% | 58.2% | 33.1% | 25.4% | 84.9% | 16.1% | 64.8% | 29.4% | 22.5% | 87.5% |
| 1258 | 34.3% | 60.9% | 30.0% | 17.5% | 69.5% | 6.9% | 52.9% | 28.5% | 18.1% | 84.5% | 20.5% | 59.8% | 28.0% | 18.0% | 85.3% |
| 1259 | 35.6% | 57.2% | 31.9% | 17.7% | 66.8% | 14.9% | 45.7% | 21.0% | 14.9% | 83.8% | 31.5% | 62.7% | 29.5% | 17.1% | 86.3% |
| 1260 | 20.1% | 58.9% | 39.5% | 19.2% | 67.1% | 8.4% | 59.3% | 26.1% | 19.8% | 83.9% | 18.1% | 65.4% | 34.1% | 20.8% | 85.8% |
| 1261 | 29.9% | 57.0% | 37.3% | 15.6% | 65.0% | 6.2% | 41.4% | 27.7% | 21.3% | 82.5% | 21.4% | 64.5% | 38.9% | 19.2% | 85.4% |
| 1262 | 22.2% | 59.3% | 30.8% | 21.0% | 67.3% | 7.0% | 54.9% | 22.0% | 18.5% | 83.3% | 18.7% | 65.5% | 28.8% | 19.9% | 86.0% |
| 1263 | 21.4% | 60.4% | 40.0% | 18.5% | 68.5% | 6.9% | 51.9% | 22.2% | 15.6% | 83.1% | 19.5% | 64.0% | 36.8% | 19.2% | 85.3% |
| 1264 | 29.3% | 57.6% | 36.9% | 18.1% | 66.8% | 12.0% | 49.6% | 26.0% | 26.3% | 84.4% | 26.1% | 63.4% | 34.7% | 21.9% | 86.7% |
| 1265 | 24.6% | 64.1% | 47.1% | 32.3% | 71.6% | 8.1% | 63.6% | 45.9% | 31.0% | 86.1% | 20.7% | 70.8% | 45.9% | 29.6% | 89.1% |
| 1266 | 16.8% | 62.5% | 38.3% | 15.3% | 70.9% | 6.4% | 44.7% | 26.8% | 19.3% | 85.1% | 16.1% | 64.6% | 36.3% | 16.6% | 87.2% |
| 1267 | 14.8% | 54.1% | 30.7% | 21.5% | 63.2% | 6.9% | 43.8% | 18.9% | 18.8% | 82.1% | 13.8% | 59.0% | 29.9% | 22.7% | 82.8% |
| 1268 | 23.3% | 62.8% | 40.1% | 17.8% | 71.1% | 6.0% | 49.6% | 20.8% | 18.8% | 84.5% | 16.2% | 63.9% | 33.1% | 18.0% | 86.5% |
| 1269 | 36.9% | 64.2% | 49.5% | 30.6% | 72.0% | 14.7% | 42.0% | 21.2% | 19.3% | 85.7% | 27.4% | 66.9% | 43.7% | 25.8% | 86.7% |
| 1270 | 37.0% | 59.8% | 43.7% | 34.0% | 67.3% | 9.2% | 58.5% | 22.6% | 18.1% | 83.1% | 27.2% | 65.6% | 40.2% | 29.5% | 86.9% |
| 1271 | 12.4% | 57.6% | 39.3% | 21.1% | 66.6% | 5.6% | 53.0% | 35.6% | 22.9% | 85.4% | 14.4% | 62.6% | 37.3% | 21.5% | 85.1% |
| 1272 | 18.6% | 58.6% | 30.1% | 21.8% | 67.3% | 7.1% | 51.9% | 23.8% | 24.3% | 85.0% | 15.4% | 65.4% | 32.9% | 26.7% | 87.3% |
| 1273 | 43.9% | 71.4% | 48.5% | 33.9% | 77.9% | 16.8% | 46.8% | 20.7% | 16.1% | 87.0% | 33.0% | 70.3% | 38.7% | 27.7% | 90.1% |
| 1274 | 31.5% | 61.3% | 44.1% | 39.6% | 68.0% | 5.9% | 58.8% | 20.6% | 15.6% | 83.3% | 21.1% | 63.8% | 39.6% | 33.9% | 83.9% |
| 1275 | 24.4% | 57.7% | 41.1% | 25.7% | 67.1% | 7.3% | 40.1% | 23.7% | 16.1% | 83.7% | 20.4% | 62.5% | 35.7% | 19.8% | 85.6% |
| 1276 | 20.7% | 61.6% | 43.7% | 36.5% | 69.8% | 5.9% | 46.1% | 23.3% | 24.3% | 84.6% | 15.8% | 63.3% | 39.6% | 32.8% | 86.0% |
| 1277 | 26.6% | 59.3% | 44.6% | 31.7% | 67.7% | 6.3% | 57.3% | 33.4% | 29.5% | 85.7% | 16.4% | 64.7% | 41.2% | 25.0% | 84.7% |
| 1278 | 35.2% | 68.1% | 53.6% | 41.8% | 74.7% | 20.4% | 41.5% | 17.8% | 15.8% | 88.8% | 28.3% | 72.6% | 47.2% | 30.8% | 89.6% |
| 1279 | 22.0% | 62.1% | 47.0% | 38.6% | 69.6% | 8.7% | 69.9% | 49.2% | 29.6% | 88.0% | 19.2% | 71.4% | 47.3% | 35.9% | 88.7% |
| 1280 | 14.8% | 61.0% | 43.2% | 37.9% | 70.1% | 5.8% | 43.0% | 20.1% | 22.6% | 86.8% | 15.1% | 65.8% | 35.6% | 31.8% | 88.4% |
| 1281 | 15.5% | 62.5% | 44.8% | 31.0% | 69.8% | 5.2% | 46.9% | 17.3% | 13.7% | 88.7% | 13.3% | 67.2% | 41.1% | 27.5% | 87.8% |
| 1282 | 30.2% | 60.8% | 45.4% | 35.4% | 69.7% | 16.4% | 40.3% | 20.9% | 18.0% | 86.2% | 26.1% | 66.4% | 43.0% | 30.4% | 88.3% |
| 1283 | 31.4% | 60.9% | 46.0% | 37.9% | 68.3% | 9.5% | 62.3% | 28.6% | 22.1% | 86.7% | 20.0% | 66.1% | 41.3% | 32.4% | 86.1% |
| 1284 | 17.5% | 58.8% | 43.4% | 32.4% | 68.7% | 6.6% | 44.4% | 31.3% | 23.2% | 87.9% | 15.6% | 63.6% | 38.2% | 25.9% | 85.8% |
| 1285 | 13.1% | 60.3% | 39.4% | 34.4% | 68.2% | 5.1% | 43.7% | 17.8% | 19.2% | 85.0% | 13.3% | 65.9% | 34.9% | 27.8% | 85.7% |
| 1286 | 15.0% | 62.1% | 44.0% | 32.8% | 69.8% | 6.2% | 47.4% | 21.6% | 26.3% | 86.0% | 15.0% | 65.1% | 38.0% | 27.6% | 85.5% |
| 1287 | 34.6% | 61.3% | 45.4% | 34.3% | 68.8% | 12.9% | 38.9% | 19.6% | 16.1% | 83.9% | 25.9% | 64.7% | 36.9% | 23.4% | 85.9% |
| 1288 | 20.4% | 63.7% | 48.8% | 44.0% | 70.0% | 6.1% | 62.3% | 18.7% | 14.7% | 85.9% | 13.3% | 71.6% | 46.9% | 40.2% | 88.5% |
| 1289 | 14.6% | 63.0% | 43.7% | 31.1% | 70.7% | 5.9% | 32.4% | 20.0% | 16.4% | 85.1% | 12.6% | 68.2% | 40.6% | 23.2% | 87.5% |
| 1290 | 20.8% | 60.9% | 43.3% | 36.2% | 68.2% | 6.1% | 56.5% | 24.0% | 24.1% | 85.9% | 15.2% | 65.6% | 38.3% | 31.7% | 85.9% |
| 1291 | 13.9% | 65.9% | 48.9% | 34.0% | 72.6% | 6.6% | 43.5% | 17.9% | 16.2% | 85.8% | 13.5% | 67.1% | 41.3% | 25.3% | 87.4% |
| 1292 | 31.1% | 62.1% | 46.0% | 33.2% | 70.7% | 16.7% | 38.5% | 18.7% | 14.9% | 85.2% | 29.7% | 68.5% | 40.2% | 25.8% | 88.5% |
| 1293 | 15.1% | 70.9% | 58.4% | 53.0% | 76.5% | 4.8% | 62.3% | 17.1% | 12.1% | 86.5% | 10.6% | 73.4% | 50.1% | 41.9% | 88.6% |
| 1294 | 19.4% | 72.9% | 58.5% | 39.3% | 78.5% | 7.0% | 52.2% | 33.5% | 22.6% | 91.5% | 13.2% | 74.7% | 55.5% | 27.5% | 90.7% |
| 1295 | 11.7% | 62.2% | 41.6% | 36.3% | 68.8% | 5.2% | 40.9% | 15.9% | 20.3% | 85.4% | 11.4% | 65.8% | 33.8% | 29.8% | 84.8% |
| 1296 | 17.6% | 69.4% | 54.6% | 41.1% | 75.2% | 6.4% | 35.8% | 18.1% | 14.8% | 84.2% | 13.7% | 59.2% | 30.5% | 21.8% | 87.6% |
| 1297 | 23.7% | 60.8% | 43.8% | 29.0% | 69.0% | 13.7% | 40.8% | 19.9% | 17.3% | 85.6% | 21.6% | 65.5% | 40.2% | 25.5% | 88.0% |
| 1298 | 18.0% | 61.9% | 49.2% | 42.5% | 69.0% | 7.1% | 62.6% | 18.0% | 13.7% | 84.9% | 13.5% | 68.2% | 45.3% | 35.6% | 87.1% |
| 1299 | 18.6% | 59.3% | 43.5% | 31.8% | 67.1% | 4.9% | 29.3% | 19.7% | 17.0% | 83.1% | 13.5% | 62.9% | 42.0% | 23.8% | 83.4% |
| 1300 | 12.5% | 59.7% | 41.0% | 35.1% | 68.3% | 4.6% | 41.0% | 17.3% | 19.3% | 87.1% | 13.8% | 67.2% | 37.5% | 30.5% | 86.6% |
| 1301 | 16.7% | 60.6% | 44.4% | 33.2% | 68.4% | 5.1% | 44.4% | 20.6% | 16.8% | 88.0% | 12.8% | 67.9% | 44.3% | 28.4% | 87.8% |
| 1302 | 25.5% | 61.6% | 44.9% | 32.5% | 68.6% | 13.6% | 44.8% | 21.0% | 18.2% | 86.4% | 25.3% | 67.1% | 41.8% | 25.7% | 87.2% |
| 1303 | 21.4% | 63.5% | 48.8% | 42.4% | 70.5% | 5.9% | 56.8% | 16.9% | 12.5% | 85.2% | 13.0% | 69.6% | 42.3% | 34.2% | 87.1% |
| 1304 | 14.6% | 58.8% | 42.9% | 30.8% | 67.4% | 6.3% | 44.7% | 31.6% | 30.1% | 88.1% | 14.9% | 66.8% | 44.5% | 27.5% | 89.2% |
| 1305 | 14.9% | 59.5% | 41.3% | 34.0% | 69.0% | 5.0% | 37.8% | 17.2% | 21.6% | 85.7% | 11.5% | 66.7% | 39.7% | 37.4% | 88.3% |
| 1306 | 14.2% | 60.3% | 39.9% | 29.0% | 68.0% | 6.3% | 35.2% | 17.0% | 17.8% | 85.2% | 12.6% | 64.1% | 31.7% | 23.3% | 86.7% |
| 1307 | 29.0% | 62.7% | 47.1% | 33.8% | 71.8% | 11.6% | 36.9% | 19.4% | 18.2% | 85.1% | 21.8% | 67.9% | 44.8% | 28.8% | 89.0% |
| 1308 | 17.3% | 61.1% | 45.8% | 39.8% | 68.2% | 7.0% | 58.6% | 18.5% | 15.6% | 84.7% | 12.5% | 70.9% | 33.2% | 31.9% | 88.0% |
| 1309 | 14.2% | 61.0% | 40.9% | 29.6% | 69.5% | 5.5% | 34.5% | 23.2% | 17.2% | 87.4% | 12.9% | 65.7% | 45.1% | 26.2% | 89.1% |
| 1310 | 17.6% | 58.8% | 43.2% | 37.4% | 67.5% | 4.8% | 32.7% | 15.7% | 19.5% | 84.4% | 12.5% | 68.0% | 40.4% | 37.7% | 88.0% |
| 1311 | 13.9% | 61.9% | 40.2% | 30.3% | 69.7% | 4.7% | 42.0% | 21.6% | 15.6% | 85.3% | 11.9% | 64.5% | 31.8% | 23.6% | 88.0% |
| 1312 | 32.6% | 65.4% | 46.7% | 35.8% | 71.7% | 13.3% | 35.4% | 17.3% | 14.2% | 88.2% | 34.8% | 60.2% | 28.3% | 25.3% | 92.1% |
| 1313 | 25.2% | 58.1% | 39.0% | 36.1% | 65.6% | 6.0% | 63.4% | 17.5% | 14.3% | 85.1% | 15.9% | 69.5% | 28.0% | 24.8% | 87.1% |
| 1314 | 14.7% | 61.4% | 41.1% | 26.8% | 70.0% | 4.8% | 41.7% | 36.6% | 27.7% | 88.3% | 13.8% | 60.9% | 44.9% | 25.5% | 89.9% |
| 1315 | 14.3% | 57.0% | 35.7% | 31.4% | 66.1% | 5.5% | 38.0% | 16.5% | 20.2% | 85.1% | 13.2% | 61.2% | 28.3% | 30.2% | 87.5% |
| 1316 | 23.5% | 67.3% | 50.2% | 36.0% | 73.9% | 6.4% | 49.2% | 22.0% | 16.2% | 88.5% | 18.6% | 64.6% | 30.6% | 21.7% | 90.8% |
| 1317 | 37.7% | 64.1% | 41.7% | 29.9% | 72.4% | 15.5% | 39.6% | 20.2% | 18.3% | 87.9% | 37.1% | 61.1% | 28.2% | 23.0% | 89.1% |
| 1318 | 28.1% | 63.9% | 46.9% | 40.2% | 71.0% | 7.5% | 67.0% | 27.7% | 18.4% | 88.0% | 19.1% | 73.2% | 36.6% | 29.7% | 89.7% |
| 1319 | 22.8% | 61.4% | 42.3% | 24.4% | 68.9% | 5.2% | 32.4% | 20.7% | 15.9% | 84.9% | 21.1% | 58.7% | 31.4% | 17.0% | 89.0% |
| 1320 | 14.4% | 56.9% | 36.4% | 32.5% | 65.7% | 6.3% | 35.2% | 14.6% | 15.3% | 82.8% | 11.9% | 59.2% | 25.0% | 26.9% | 87.9% |
| 1321 | 16.2% | 60.8% | 36.3% | 24.0% | 68.3% | 6.3% | 42.1% | 18.5% | 13.8% | 84.4% | 14.7% | 58.5% | 25.3% | 20.3% | 85.9% |
| 1322 | 40.4% | 64.5% | 40.6% | 28.4% | 71.5% | 14.7% | 38.3% | 19.9% | 27.2% | 89.8% | 33.9% | 58.0% | 28.3% | 26.9% | 91.3% |
| 1323 | 39.7% | 67.6% | 43.7% | 39.6% | 73.5% | 5.6% | 54.3% | 16.9% | 12.8% | 83.2% | 16.1% | 65.2% | 22.9% | 16.3% | 85.2% |
| 1324 | 34.2% | 70.3% | 49.5% | 26.7% | 76.7% | 5.3% | 37.0% | 30.4% | 15.1% | 88.6% | 32.3% | 57.6% | 35.1% | 14.4% | 91.4% |
| 1325 | 13.5% | 60.2% | 31.7% | 30.2% | 68.0% | 4.1% | 41.8% | 15.2% | 25.0% | 89.4% | 11.2% | 67.8% | 25.0% | 27.8% | 89.3% |
| 1326 | 21.4% | 64.6% | 38.7% | 22.0% | 71.7% | 4.5% | 36.4% | 14.6% | 12.3% | 88.8% | 13.6% | 60.2% | 23.7% | 17.3% | 89.1% |
| 1327 | 40.8% | 61.5% | 36.5% | 25.0% | 69.2% | 12.3% | 38.0% | 18.4% | 13.9% | 84.2% | 34.0% | 59.8% | 27.6% | 19.3% | 86.3% |
| 1328 | 33.0% | 64.7% | 44.7% | 37.7% | 70.8% | 9.3% | 70.7% | 36.2% | 30.2% | 88.6% | 22.3% | 75.3% | 34.1% | 30.0% | 90.2% |
| 1329 | 24.3% | 58.6% | 39.9% | 20.9% | 67.0% | 7.3% | 39.2% | 25.4% | 18.3% | 85.1% | 21.6% | 62.7% | 34.3% | 19.3% | 87.3% |
| 1330 | 18.7% | 59.4% | 33.5% | 29.2% | 68.3% | 5.0% | 34.3% | 15.5% | 20.1% | 86.3% | 14.2% | 65.0% | 27.6% | 25.1% | 86.3% |
| 1331 | 18.7% | 58.8% | 33.4% | 18.6% | 67.0% | 4.7% | 40.5% | 17.0% | 14.5% | 86.5% | 13.6% | 58.9% | 24.7% | 17.2% | 85.5% |
| 1332 | 28.8% | 62.9% | 41.1% | 34.6% | 70.0% | 6.5% | 62.8% | 18.7% | 14.2% | 86.2% | 17.6% | 68.9% | 26.3% | 19.6% | 86.5% |
| 1333 | 20.1% | 66.4% | 39.9% | 17.6% | 73.3% | 4.9% | 38.6% | 32.5% | 14.9% | 87.8% | 14.9% | 66.4% | 37.0% | 15.7% | 87.4% |
| 1334 | 17.8% | 62.2% | 34.1% | 31.4% | 70.1% | 4.9% | 38.0% | 16.4% | 20.9% | 85.6% | 14.9% | 65.7% | 25.0% | 27.7% | 87.4% |
| 1335 | 18.4% | 63.3% | 33.9% | 21.9% | 71.3% | 5.8% | 60.9% | 33.1% | 18.7% | 88.0% | 12.9% | 66.2% | 32.7% | 19.8% | 88.3% |
| 1336 | 26.9% | 60.9% | 32.9% | 27.1% | 68.6% | 7.8% | 62.4% | 18.2% | 13.5% | 86.6% | 20.7% | 69.4% | 28.2% | 22.4% | 89.5% |
| 1337 | 24.5% | 60.7% | 37.3% | 19.8% | 71.4% | 8.2% | 38.8% | 20.7% | 15.3% | 87.6% | 19.1% | 60.2% | 32.8% | 17.7% | 88.9% |
| 1338 | 18.3% | 57.5% | 31.8% | 29.7% | 68.7% | 5.2% | 58.4% | 19.7% | 26.5% | 87.6% | 17.4% | 67.0% | 30.0% | 30.9% | 90.3% |
| 1339 | 18.7% | 57.0% | 31.3% | 20.7% | 68.2% | 7.9% | 59.6% | 21.0% | 14.6% | 87.4% | 19.3% | 64.5% | 27.9% | 21.0% | 87.8% |
| 1340 | 12.4% | 73.3% | 20.9% | 23.0% | 80.5% | 8.7% | 70.5% | 13.6% | 9.0% | 89.0% | 12.5% | 73.9% | 16.8% | 12.0% | 90.8% |
| 1341 | 18.6% | 61.2% | 37.3% | 13.4% | 80.9% | 8.1% | 42.3% | 17.7% | 9.6% | 90.4% | 17.5% | 49.8% | 24.1% | 11.1% | 91.0% |
| 1342 | 17.0% | 65.0% | 29.9% | 39.3% | 76.2% | 6.7% | 39.8% | 14.3% | 15.3% | 88.1% | 15.6% | 55.0% | 20.0% | 19.8% | 89.5% |
| 1343 | 10.0% | 65.1% | 27.2% | 27.2% | 80.5% | 5.2% | 34.8% | 15.3% | 12.7% | 85.2% | 10.9% | 46.9% | 20.2% | 13.8% | 88.5% |
| 1344 | 34.5% | 63.5% | 24.5% | 22.7% | 71.9% | 9.2% | 60.0% | 16.5% | 12.1% | 84.7% | 20.5% | 67.3% | 23.2% | 16.8% | 88.2% |
| 1345 | 12.4% | 61.0% | 29.2% | 15.1% | 71.0% | 6.3% | 33.3% | 15.7% | 12.0% | 83.7% | 14.5% | 55.1% | 26.1% | 14.0% | 86.4% |
| 1346 | 17.6% | 53.3% | 25.2% | 29.0% | 70.7% | 7.1% | 41.7% | 16.9% | 14.4% | 84.5% | 15.0% | 51.7% | 22.8% | 19.0% | 86.3% |
| 1347 | 15.5% | 45.4% | 21.7% | 14.9% | 66.2% | 6.5% | 36.0% | 17.4% | 13.0% | 80.2% | 12.9% | 44.5% | 20.6% | 13.4% | 82.3% |
| 1348 | 25.5% | 60.2% | 26.6% | 18.4% | 69.0% | 13.2% | 58.1% | 16.8% | 13.9% | 83.7% | 20.0% | 63.7% | 25.0% | 18.3% | 86.9% |
| 1349 | 31.2% | 50.8% | 29.2% | 13.5% | 69.4% | 10.9% | 39.4% | 18.3% | 13.9% | 84.3% | 25.8% | 51.2% | 25.0% | 16.2% | 85.8% |
| 1350 | 16.8% | 59.1% | 23.5% | 22.9% | 71.5% | 6.4% | 44.8% | 15.2% | 12.8% | 85.7% | 14.7% | 54.6% | 20.9% | 16.3% | 86.9% |
| 1351 | 14.6% | 56.0% | 25.9% | 16.7% | 68.8% | 7.6% | 47.2% | 19.0% | 14.4% | 85.1% | 15.2% | 52.5% | 23.3% | 17.0% | 86.9% |
| 1352 | 25.3% | 57.8% | 30.1% | 18.1% | 67.2% | 17.3% | 54.7% | 18.2% | 14.3% | 81.4% | 22.6% | 63.8% | 27.1% | 18.4% | 86.1% |
| 1353 | 14.3% | 59.8% | 33.5% | 14.0% | 70.5% | 6.5% | 56.0% | 22.7% | 15.0% | 86.3% | 14.1% | 60.5% | 28.8% | 15.5% | 87.8% |
| 1354 | 24.7% | 61.1% | 23.3% | 17.9% | 70.5% | 8.8% | 60.4% | 23.7% | 23.1% | 85.7% | 17.2% | 63.3% | 26.0% | 20.4% | 87.7% |
| 1355 | 12.4% | 64.5% | 34.6% | 19.7% | 72.3% | 5.8% | 63.7% | 24.8% | 14.5% | 86.9% | 13.2% | 68.8% | 28.1% | 17.8% | 88.7% |
| 1356 | 13.5% | 56.3% | 37.5% | 15.9% | 64.3% | 6.2% | 40.0% | 20.7% | 14.1% | 83.3% | 13.8% | 59.4% | 32.5% | 16.3% | 84.3% |
| 1357 | 14.0% | 57.6% | 26.3% | 19.3% | 66.9% | 6.2% | 50.6% | 17.8% | 16.4% | 84.3% | 14.8% | 59.7% | 25.4% | 18.5% | 86.5% |
| 1358 | 13.7% | 58.6% | 29.2% | 17.3% | 68.0% | 6.9% | 55.9% | 21.9% | 16.9% | 84.3% | 13.7% | 60.3% | 26.0% | 17.3% | 85.2% |
| 1359 | 21.8% | 59.2% | 41.5% | 21.8% | 67.8% | 11.2% | 60.5% | 35.7% | 22.2% | 86.4% | 21.8% | 66.8% | 37.5% | 21.1% | 87.6% |
| 1360 | 25.0% | 55.9% | 37.9% | 13.9% | 66.5% | 17.8% | 56.6% | 39.1% | 21.9% | 86.0% | 25.5% | 61.3% | 36.7% | 17.2% | 87.3% |
| 1361 | 13.3% | 59.4% | 26.8% | 18.6% | 68.1% | 6.1% | 51.6% | 20.2% | 19.3% | 85.1% | 12.9% | 63.1% | 28.6% | 20.7% | 85.0% |
| 1362 | 16.5% | 59.8% | 38.4% | 16.4% | 67.0% | 6.1% | 53.9% | 22.6% | 15.1% | 82.4% | 14.2% | 63.2% | 31.0% | 16.9% | 85.1% |
| 1363 | 20.5% | 57.9% | 43.4% | 30.2% | 66.1% | 6.9% | 56.8% | 20.7% | 21.7% | 83.1% | 13.6% | 65.2% | 38.0% | 26.4% | 85.5% |
| 1364 | 15.2% | 60.3% | 41.3% | 19.4% | 69.7% | 9.3% | 59.6% | 35.5% | 19.1% | 86.2% | 15.7% | 64.6% | 38.8% | 19.6% | 86.8% |
| 1365 | 13.4% | 59.1% | 37.4% | 25.5% | 66.9% | 6.3% | 33.8% | 18.0% | 30.7% | 81.6% | 13.1% | 58.2% | 31.3% | 29.3% | 84.3% |
| 1366 | 13.0% | 60.0% | 38.9% | 18.7% | 68.0% | 7.4% | 56.0% | 23.6% | 18.5% | 84.7% | 13.5% | 63.9% | 32.3% | 18.6% | 86.6% |
| 1367 | 31.7% | 61.1% | 44.5% | 35.0% | 69.1% | 11.0% | 62.8% | 34.3% | 29.3% | 85.1% | 25.9% | 66.8% | 41.0% | 30.7% | 87.5% |
| 1368 | 18.0% | 59.2% | 41.6% | 21.6% | 68.5% | 6.6% | 54.6% | 30.6% | 23.7% | 84.7% | 15.6% | 64.9% | 41.2% | 21.3% | 88.7% |
| 1369 | 11.4% | 65.0% | 39.5% | 30.7% | 73.3% | 5.3% | 55.7% | 23.2% | 23.5% | 85.8% | 12.9% | 68.0% | 36.0% | 27.9% | 89.2% |
| 1370 | 15.5% | 59.8% | 43.8% | 27.0% | 66.7% | 7.9% | 50.9% | 22.1% | 18.8% | 83.1% | 15.2% | 64.3% | 37.8% | 24.9% | 84.7% |
| 1371 | 31.5% | 61.3% | 45.1% | 38.0% | 69.6% | 9.6% | 58.2% | 20.7% | 16.1% | 84.6% | 20.5% | 65.5% | 40.6% | 32.7% | 86.5% |
| 1372 | 14.9% | 61.5% | 45.7% | 33.1% | 69.4% | 6.9% | 43.0% | 24.6% | 20.8% | 86.1% | 14.6% | 62.5% | 39.3% | 24.3% | 85.9% |
| 1373 | 13.4% | 57.2% | 40.3% | 32.5% | 65.6% | 6.2% | 44.1% | 18.2% | 20.0% | 84.6% | 13.7% | 63.4% | 38.5% | 30.9% | 87.0% |
| 1374 | 9.6% | 68.8% | 55.7% | 41.0% | 74.9% | 5.2% | 48.0% | 16.0% | 13.3% | 87.5% | 10.3% | 66.1% | 41.4% | 29.4% | 85.9% |
| 1375 | 17.0% | 58.4% | 42.8% | 37.8% | 67.1% | 6.8% | 62.2% | 22.7% | 18.2% | 85.1% | 15.3% | 66.4% | 40.6% | 35.1% | 85.2% |
| 1376 | 12.1% | 58.1% | 43.7% | 32.2% | 69.2% | 5.4% | 32.1% | 18.6% | 30.8% | 86.4% | 12.1% | 59.2% | 35.0% | 26.7% | 87.0% |
| 1377 | 13.3% | 58.0% | 41.7% | 36.1% | 67.0% | 5.8% | 42.7% | 16.2% | 19.9% | 84.9% | 13.5% | 62.3% | 35.7% | 30.2% | 83.9% |
| 1378 | 13.1% | 58.0% | 42.7% | 30.0% | 66.8% | 7.3% | 55.4% | 25.1% | 16.7% | 84.8% | 13.5% | 63.4% | 39.4% | 28.7% | 86.2% |
| 1379 | 22.0% | 64.1% | 49.8% | 43.2% | 71.3% | 8.3% | 66.4% | 23.0% | 27.5% | 86.3% | 15.9% | 70.2% | 44.8% | 40.7% | 88.8% |
| 1380 | 18.6% | 57.2% | 40.2% | 28.8% | 67.2% | 6.8% | 34.3% | 21.5% | 16.2% | 87.6% | 19.6% | 64.3% | 38.3% | 23.7% | 87.3% |
| 1381 | 23.9% | 57.0% | 38.7% | 33.9% | 65.6% | 6.4% | 45.4% | 16.9% | 19.1% | 84.3% | 21.8% | 61.6% | 35.0% | 30.4% | 85.3% |
| 1382 | 17.8% | 61.9% | 46.1% | 31.9% | 70.1% | 9.4% | 63.3% | 30.7% | 16.0% | 87.4% | 16.2% | 67.4% | 40.9% | 22.9% | 87.9% |
| 1383 | 21.2% | 64.1% | 50.1% | 44.1% | 71.3% | 8.6% | 65.0% | 17.6% | 15.5% | 85.9% | 18.5% | 71.5% | 45.4% | 41.6% | 89.0% |
| 1384 | 13.6% | 57.0% | 40.7% | 30.8% | 66.8% | 6.7% | 34.3% | 18.6% | 17.4% | 83.9% | 12.6% | 61.6% | 38.4% | 24.2% | 86.0% |
| 1385 | 18.2% | 63.3% | 46.0% | 39.6% | 71.1% | 7.4% | 62.7% | 22.3% | 21.6% | 87.2% | 15.2% | 69.0% | 40.0% | 36.4% | 89.1% |
| 1386 | 10.6% | 66.1% | 50.2% | 39.0% | 73.6% | 5.1% | 62.0% | 26.3% | 16.5% | 88.4% | 11.2% | 69.3% | 42.9% | 28.5% | 89.2% |
| 1387 | 12.6% | 59.3% | 45.1% | 37.7% | 66.8% | 5.6% | 49.3% | 15.7% | 19.2% | 84.6% | 11.5% | 65.8% | 34.3% | 32.3% | 86.7% |
| 1388 | 11.2% | 60.8% | 46.0% | 33.6% | 69.5% | 13.3% | 50.9% | 28.9% | 16.8% | 85.7% | 16.2% | 63.1% | 38.2% | 23.5% | 85.2% |
| 1389 | 11.2% | 64.0% | 44.1% | 38.4% | 71.7% | 4.4% | 61.6% | 17.5% | 19.4% | 86.3% | 11.5% | 68.5% | 35.7% | 33.1% | 88.5% |
| 1390 | 11.9% | 68.4% | 47.6% | 33.1% | 75.3% | 6.7% | 57.0% | 22.4% | 18.9% | 88.8% | 11.1% | 70.6% | 35.7% | 26.4% | 90.4% |
| 1391 | 17.1% | 69.8% | 58.4% | 53.5% | 76.0% | 10.4% | 65.8% | 15.6% | 11.6% | 86.8% | 13.7% | 73.5% | 45.7% | 41.6% | 89.2% |
| 1392 | 12.5% | 61.5% | 43.9% | 30.9% | 69.2% | 4.7% | 62.6% | 41.3% | 22.1% | 87.3% | 12.8% | 67.9% | 46.0% | 26.2% | 88.3% |
| 1393 | 10.1% | 70.3% | 53.4% | 36.8% | 76.6% | 4.4% | 42.4% | 16.1% | 20.7% | 86.1% | 9.9% | 68.7% | 33.7% | 25.3% | 89.6% |
| 1394 | 25.6% | 60.7% | 44.5% | 39.1% | 68.2% | 9.4% | 61.6% | 16.8% | 15.1% | 84.9% | 19.2% | 70.4% | 43.4% | 36.5% | 89.1% |
| 1395 | 15.8% | 62.5% | 45.9% | 31.7% | 70.7% | 7.8% | 34.8% | 20.4% | 16.0% | 86.3% | 15.9% | 68.4% | 44.4% | 23.0% | 89.2% |
| 1396 | 13.6% | 58.4% | 40.3% | 35.7% | 67.5% | 6.2% | 44.0% | 18.7% | 21.7% | 85.8% | 12.0% | 65.9% | 38.5% | 35.5% | 87.9% |
| 1397 | 12.8% | 62.7% | 44.8% | 30.4% | 71.0% | 5.5% | 39.7% | 18.6% | 16.0% | 85.8% | 12.2% | 65.3% | 35.7% | 25.4% | 88.6% |
| 1398 | 15.7% | 61.9% | 44.2% | 37.0% | 69.7% | 8.1% | 64.5% | 25.1% | 17.2% | 86.8% | 14.1% | 70.2% | 32.2% | 25.6% | 87.9% |
| 1399 | 11.0% | 63.4% | 47.5% | 36.6% | 72.5% | 4.8% | 29.1% | 16.3% | 13.7% | 86.8% | 11.2% | 63.9% | 40.8% | 23.7% | 90.0% |
| 1400 | 19.9% | 61.2% | 41.7% | 36.2% | 69.5% | 6.5% | 43.4% | 17.4% | 17.0% | 85.5% | 15.3% | 65.2% | 35.7% | 37.0% | 88.5% |
| 1401 | 28.0% | 63.4% | 44.2% | 37.9% | 71.1% | 9.5% | 57.1% | 17.3% | 15.2% | 83.8% | 19.3% | 68.7% | 27.6% | 22.8% | 89.1% |
| 1402 | 15.7% | 63.0% | 43.9% | 29.1% | 72.0% | 6.9% | 36.2% | 22.1% | 15.9% | 86.0% | 14.4% | 62.6% | 37.1% | 18.2% | 89.5% |
| 1403 | 11.6% | 59.4% | 36.1% | 31.6% | 67.8% | 11.3% | 60.9% | 22.2% | 23.1% | 86.5% | 13.4% | 66.0% | 27.8% | 26.7% | 88.7% |
| 1404 | 13.4% | 60.4% | 41.2% | 29.1% | 68.9% | 5.2% | 62.3% | 22.3% | 15.6% | 86.6% | 12.2% | 66.6% | 30.3% | 25.9% | 88.4% |
| 1405 | 17.3% | 64.6% | 44.3% | 36.7% | 72.7% | 9.3% | 69.4% | 22.4% | 13.1% | 90.0% | 14.3% | 74.0% | 28.4% | 21.7% | 89.8% |
| 1406 | 12.4% | 58.2% | 40.1% | 23.1% | 66.6% | 4.7% | 29.7% | 18.4% | 17.7% | 85.7% | 12.3% | 55.2% | 30.0% | 18.4% | 87.0% |
| 1407 | 12.1% | 60.0% | 39.7% | 32.3% | 67.7% | 4.8% | 43.4% | 19.0% | 28.6% | 86.5% | 11.6% | 60.4% | 25.6% | 27.8% | 88.4% |
| 1408 | 14.8% | 58.1% | 37.3% | 25.3% | 67.4% | 5.5% | 44.4% | 19.1% | 14.6% | 85.7% | 13.7% | 57.7% | 26.1% | 18.7% | 87.2% |
| 1409 | 17.4% | 63.8% | 43.2% | 38.2% | 70.9% | 5.9% | 66.3% | 16.0% | 12.1% | 87.5% | 12.1% | 70.3% | 22.2% | 17.8% | 86.7% |
| 1410 | 11.1% | 67.6% | 49.2% | 22.4% | 74.2% | 4.2% | 22.3% | 14.4% | 12.2% | 86.9% | 9.5% | 55.7% | 27.9% | 13.5% | 86.4% |
| 1411 | 31.2% | 78.2% | 48.5% | 49.2% | 83.1% | 4.2% | 31.5% | 9.5% | 16.7% | 90.9% | 17.9% | 63.4% | 18.2% | 29.5% | 92.3% |
| 1412 | 8.9% | 75.3% | 46.1% | 28.4% | 81.4% | 6.2% | 45.1% | 13.7% | 10.1% | 92.4% | 9.5% | 60.3% | 20.2% | 15.9% | 91.6% |
| 1413 | 33.0% | 63.1% | 42.1% | 36.6% | 70.3% | 6.7% | 64.9% | 15.6% | 15.2% | 87.2% | 17.7% | 69.6% | 24.5% | 20.6% | 87.5% |
| 1414 | 12.4% | 56.4% | 36.6% | 18.9% | 65.8% | 5.3% | 30.1% | 17.7% | 16.7% | 84.8% | 11.8% | 58.8% | 30.1% | 16.0% | 85.2% |
| 1415 | 16.3% | 64.1% | 38.8% | 34.1% | 71.7% | 6.0% | 37.3% | 15.6% | 19.4% | 88.4% | 13.5% | 63.4% | 25.9% | 26.3% | 89.1% |
| 1416 | 14.1% | 64.6% | 37.6% | 22.7% | 72.2% | 4.9% | 53.2% | 20.6% | 22.9% | 88.9% | 18.1% | 64.0% | 25.6% | 19.3% | 88.5% |
| 1417 | 34.4% | 60.9% | 37.7% | 35.3% | 68.5% | 8.3% | 60.9% | 17.5% | 14.5% | 83.7% | 17.6% | 64.3% | 24.3% | 18.8% | 82.6% |
| 1418 | 28.6% | 62.6% | 36.8% | 17.9% | 71.9% | 8.4% | 35.6% | 20.6% | 13.8% | 87.7% | 29.0% | 63.7% | 31.8% | 16.3% | 88.9% |
| 1419 | 17.0% | 63.6% | 34.7% | 29.6% | 72.1% | 10.3% | 68.1% | 47.0% | 50.0% | 88.2% | 13.6% | 73.7% | 47.1% | 46.1% | 91.3% |
| 1420 | 17.4% | 63.3% | 32.4% | 20.8% | 70.4% | 11.0% | 66.0% | 44.8% | 25.4% | 87.1% | 14.5% | 72.0% | 44.9% | 22.3% | 89.6% |
| 1421 | 11.0% | 55.2% | 23.5% | 25.5% | 76.0% | 5.3% | 46.6% | 17.0% | 13.1% | 87.8% | 9.8% | 52.9% | 21.5% | 20.3% | 88.6% |
| 1422 | 11.5% | 60.4% | 29.6% | 26.1% | 70.8% | 11.5% | 61.4% | 18.4% | 13.3% | 85.3% | 13.2% | 68.1% | 24.7% | 21.0% | 87.4% |
| 1423 | 12.6% | 55.6% | 37.1% | 18.1% | 69.5% | 6.7% | 46.6% | 25.2% | 17.6% | 86.2% | 11.1% | 60.1% | 34.4% | 16.8% | 87.0% |
| 1424 | 11.8% | 61.3% | 31.8% | 30.0% | 73.1% | 4.8% | 63.2% | 16.3% | 16.9% | 88.9% | 11.8% | 65.5% | 26.8% | 27.9% | 89.5% |
| 1425 | 11.6% | 58.2% | 30.8% | 21.0% | 71.0% | 6.1% | 68.8% | 50.7% | 16.1% | 87.4% | 13.0% | 70.5% | 42.0% | 18.9% | 89.5% |
| 1426 | 8.7% | 50.7% | 19.4% | 25.1% | 77.7% | 7.4% | 44.8% | 17.4% | 13.3% | 88.3% | 10.4% | 51.0% | 20.2% | 15.8% | 88.0% |
| 1427 | 7.3% | 67.4% | 19.6% | 17.9% | 79.6% | 12.2% | 76.7% | 11.8% | 8.6% | 90.6% | 8.7% | 75.9% | 15.3% | 11.2% | 91.5% |
| 1428 | 11.2% | 44.6% | 35.2% | 16.4% | 67.5% | 7.1% | 33.3% | 18.4% | 11.9% | 84.0% | 12.0% | 45.7% | 27.7% | 16.5% | 85.8% |
| 1429 | 10.5% | 58.4% | 31.0% | 32.5% | 73.5% | 6.1% | 57.7% | 17.5% | 17.7% | 87.3% | 11.3% | 59.1% | 22.8% | 21.4% | 88.5% |
| 1430 | 8.6% | 58.5% | 22.4% | 16.7% | 77.1% | 15.5% | 74.6% | 23.9% | 9.9% | 91.0% | 11.7% | 72.8% | 21.6% | 11.2% | 91.4% |
| 1431 | 11.7% | 49.8% | 21.4% | 17.5% | 69.6% | 6.7% | 39.2% | 17.4% | 13.8% | 84.7% | 11.3% | 47.7% | 22.0% | 15.8% | 84.8% |
| 1432 | 13.1% | 59.3% | 22.6% | 15.6% | 69.1% | 9.4% | 63.9% | 23.5% | 14.2% | 86.2% | 12.4% | 65.1% | 23.7% | 15.3% | 87.2% |
| 1433 | 10.7% | 53.4% | 31.3% | 12.5% | 70.7% | 8.3% | 52.2% | 27.3% | 13.4% | 85.5% | 11.8% | 51.9% | 27.5% | 13.2% | 86.1% |
| 1434 | 10.8% | 59.4% | 24.8% | 21.6% | 74.4% | 5.9% | 55.6% | 17.4% | 15.7% | 88.2% | 11.6% | 56.6% | 22.4% | 19.2% | 88.6% |
| 1435 | 9.8% | 56.9% | 22.3% | 14.2% | 72.0% | 9.3% | 57.2% | 18.2% | 11.7% | 87.3% | 12.3% | 55.7% | 22.9% | 14.7% | 87.6% |
| 1436 | 9.6% | 58.5% | 23.2% | 17.7% | 72.6% | 8.5% | 44.6% | 17.2% | 11.9% | 84.0% | 12.8% | 52.1% | 23.1% | 15.7% | 86.5% |
| 1437 | 12.8% | 61.8% | 28.7% | 21.4% | 69.7% | 11.9% | 66.3% | 29.2% | 20.6% | 87.2% | 12.9% | 68.9% | 26.9% | 19.4% | 86.9% |
| 1438 | 10.5% | 57.9% | 32.8% | 12.6% | 72.8% | 17.0% | 53.7% | 24.3% | 15.6% | 86.2% | 16.6% | 56.3% | 30.6% | 15.6% | 87.8% |
| 1439 | 11.6% | 59.3% | 26.9% | 19.0% | 72.2% | 5.9% | 59.9% | 16.7% | 14.3% | 85.7% | 13.7% | 62.3% | 24.6% | 18.7% | 87.7% |
| 1440 | 11.2% | 54.7% | 26.9% | 17.3% | 67.3% | 9.8% | 51.2% | 20.4% | 13.7% | 81.5% | 13.5% | 56.2% | 24.5% | 16.0% | 83.1% |
| 1441 | 11.0% | 59.2% | 32.3% | 18.8% | 71.3% | 5.8% | 61.5% | 37.7% | 21.3% | 85.8% | 13.0% | 62.1% | 31.5% | 19.8% | 86.7% |
| 1442 | 11.6% | 60.6% | 39.3% | 19.4% | 69.0% | 8.7% | 63.3% | 45.4% | 28.5% | 84.9% | 14.2% | 67.4% | 42.6% | 19.6% | 86.6% |
| 1443 | 12.5% | 57.4% | 32.5% | 14.3% | 68.0% | 8.0% | 44.7% | 22.5% | 14.9% | 84.8% | 13.7% | 57.6% | 28.8% | 15.5% | 87.1% |
| 1444 | 11.9% | 60.7% | 29.2% | 20.2% | 70.1% | 8.5% | 56.6% | 18.8% | 15.6% | 83.7% | 13.6% | 63.7% | 26.8% | 19.4% | 87.6% |
| 1445 | 9.3% | 64.8% | 29.9% | 15.3% | 74.0% | 9.5% | 66.7% | 25.4% | 12.5% | 86.7% | 12.7% | 65.7% | 25.2% | 14.7% | 87.5% |
| 1446 | 11.6% | 54.7% | 32.2% | 19.1% | 68.8% | 6.3% | 55.8% | 25.1% | 18.3% | 85.2% | 13.7% | 60.7% | 30.7% | 20.4% | 86.6% |
| 1447 | 12.9% | 57.4% | 32.9% | 16.1% | 66.0% | 8.3% | 62.5% | 21.0% | 14.9% | 83.7% | 14.8% | 63.5% | 27.0% | 16.3% | 84.2% |
| 1448 | 14.0% | 55.6% | 36.4% | 15.8% | 67.1% | 6.3% | 47.6% | 29.7% | 16.8% | 81.2% | 14.5% | 59.4% | 33.0% | 16.7% | 84.3% |
| 1449 | 12.2% | 50.1% | 26.0% | 18.8% | 64.9% | 6.6% | 48.0% | 20.0% | 19.2% | 82.2% | 12.7% | 55.4% | 26.3% | 19.8% | 83.7% |
| 1450 | 13.2% | 59.3% | 38.9% | 17.8% | 68.7% | 7.9% | 54.9% | 21.8% | 15.5% | 84.4% | 14.3% | 62.2% | 31.6% | 17.0% | 85.7% |
| 1451 | 9.0% | 65.5% | 41.3% | 33.8% | 76.3% | 6.3% | 49.1% | 23.4% | 21.5% | 85.4% | 10.6% | 63.2% | 35.0% | 30.3% | 88.7% |
| 1452 | 12.0% | 59.1% | 40.2% | 21.2% | 68.0% | 8.6% | 60.3% | 28.3% | 20.6% | 84.9% | 14.0% | 65.8% | 35.4% | 20.2% | 86.8% |
| 1453 | 13.8% | 56.8% | 40.4% | 19.4% | 67.4% | 6.5% | 49.6% | 27.2% | 20.1% | 82.6% | 14.0% | 60.8% | 37.0% | 19.7% | 87.0% |
| 1454 | 9.7% | 62.2% | 29.0% | 21.4% | 72.1% | 5.6% | 53.4% | 21.1% | 23.2% | 84.2% | 11.4% | 62.9% | 26.7% | 20.9% | 87.3% |
| 1455 | 12.2% | 58.7% | 39.7% | 20.0% | 69.0% | 5.8% | 56.5% | 27.9% | 24.9% | 86.7% | 13.7% | 62.0% | 34.2% | 22.8% | 86.9% |
| 1456 | 14.6% | 60.4% | 39.7% | 24.6% | 69.5% | 7.8% | 51.5% | 29.1% | 30.3% | 84.7% | 13.9% | 61.4% | 39.0% | 30.6% | 87.3% |
| 1457 | 11.3% | 57.4% | 40.0% | 29.6% | 66.2% | 8.7% | 58.7% | 25.1% | 18.4% | 84.7% | 13.8% | 63.1% | 37.2% | 26.5% | 84.6% |
| 1458 | 11.6% | 63.4% | 48.1% | 22.1% | 72.5% | 6.2% | 45.2% | 27.0% | 19.0% | 85.0% | 12.7% | 65.8% | 43.4% | 20.5% | 87.3% |
| 1459 | 12.2% | 62.6% | 41.5% | 33.5% | 71.9% | 5.0% | 56.1% | 31.4% | 33.1% | 85.0% | 12.5% | 66.2% | 39.0% | 32.5% | 88.5% |
| 1460 | 11.9% | 64.0% | 46.2% | 20.7% | 71.9% | 5.8% | 65.0% | 47.2% | 20.4% | 85.5% | 13.1% | 69.2% | 42.8% | 21.9% | 87.7% |
| 1461 | 14.9% | 65.5% | 48.4% | 33.0% | 74.3% | 9.7% | 58.9% | 29.5% | 28.4% | 85.5% | 15.2% | 66.5% | 40.4% | 29.2% | 88.0% |
| 1462 | 11.5% | 64.3% | 49.1% | 39.4% | 72.8% | 9.0% | 65.9% | 49.3% | 35.9% | 86.6% | 12.2% | 70.2% | 47.8% | 34.9% | 88.1% |
| 1463 | 8.4% | 71.4% | 59.2% | 45.6% | 80.2% | 4.6% | 68.7% | 35.9% | 31.5% | 89.4% | 9.7% | 68.7% | 44.9% | 29.3% | 89.7% |
| 1464 | 11.4% | 60.1% | 39.5% | 32.2% | 68.1% | 5.8% | 41.0% | 16.3% | 16.1% | 82.6% | 11.1% | 60.0% | 32.1% | 27.0% | 82.1% |
| 1465 | 8.6% | 66.5% | 47.1% | 31.0% | 74.7% | 5.9% | 58.9% | 34.5% | 27.7% | 84.4% | 8.8% | 66.0% | 42.7% | 30.2% | 87.1% |
| 1466 | 11.0% | 63.0% | 44.2% | 35.0% | 71.6% | 4.7% | 63.8% | 41.2% | 44.0% | 85.9% | 10.4% | 66.4% | 45.6% | 39.5% | 88.9% |
| 1467 | 12.1% | 63.4% | 48.2% | 41.8% | 70.3% | 9.4% | 66.4% | 33.7% | 24.5% | 86.4% | 11.5% | 68.2% | 42.7% | 36.2% | 87.2% |
| 1468 | 11.7% | 60.4% | 42.3% | 30.2% | 69.2% | 6.2% | 39.4% | 20.8% | 15.9% | 84.3% | 12.3% | 64.9% | 39.2% | 24.6% | 87.0% |
| 1469 | 10.3% | 65.1% | 46.2% | 43.0% | 73.1% | 5.1% | 43.3% | 17.8% | 26.0% | 86.8% | 10.2% | 66.7% | 40.6% | 37.0% | 86.8% |
| 1470 | 13.4% | 60.5% | 40.3% | 30.0% | 68.5% | 7.4% | 62.0% | 24.6% | 29.4% | 86.6% | 12.4% | 65.1% | 38.4% | 31.3% | 87.3% |
| 1471 | 9.7% | 60.3% | 41.2% | 36.8% | 69.4% | 6.5% | 46.8% | 23.4% | 20.3% | 84.8% | 11.3% | 64.4% | 34.3% | 31.6% | 85.9% |
| 1472 | 13.5% | 61.6% | 44.9% | 38.3% | 69.5% | 9.9% | 60.9% | 21.0% | 14.3% | 85.0% | 13.4% | 67.6% | 36.3% | 30.7% | 87.5% |
| 1473 | 12.2% | 56.9% | 39.9% | 29.5% | 65.9% | 6.4% | 45.4% | 25.3% | 16.6% | 84.4% | 13.4% | 63.0% | 35.8% | 23.9% | 85.3% |
| 1474 | 12.6% | 56.6% | 39.2% | 34.6% | 67.0% | 6.4% | 37.8% | 18.5% | 17.8% | 82.5% | 11.0% | 62.0% | 34.2% | 30.7% | 85.8% |
| 1475 | 11.4% | 64.9% | 48.1% | 36.9% | 72.9% | 5.3% | 62.9% | 30.0% | 21.9% | 86.3% | 11.4% | 67.6% | 39.9% | 30.0% | 87.4% |
| 1476 | 11.9% | 60.4% | 36.2% | 31.2% | 69.6% | 6.4% | 42.0% | 19.1% | 15.9% | 85.0% | 13.0% | 62.9% | 30.3% | 27.7% | 86.5% |
| 1477 | 12.8% | 59.8% | 44.5% | 37.2% | 68.4% | 6.6% | 66.6% | 48.2% | 40.2% | 86.4% | 12.1% | 69.2% | 46.9% | 37.5% | 89.2% |
| 1478 | 10.7% | 67.5% | 52.5% | 37.3% | 74.5% | 5.7% | 69.9% | 50.1% | 25.9% | 88.8% | 10.2% | 71.2% | 49.4% | 27.7% | 89.4% |
| 1479 | 9.4% | 68.0% | 50.6% | 44.4% | 75.9% | 4.4% | 71.3% | 45.5% | 41.5% | 89.4% | 9.3% | 73.7% | 48.7% | 41.5% | 90.1% |
| 1480 | 11.0% | 62.9% | 41.9% | 30.4% | 71.2% | 6.6% | 53.2% | 18.9% | 16.9% | 87.3% | 10.7% | 65.4% | 34.0% | 27.4% | 87.1% |
| 1481 | 12.5% | 61.8% | 39.1% | 27.3% | 71.5% | 6.8% | 49.1% | 23.5% | 31.3% | 86.7% | 10.9% | 63.1% | 37.0% | 33.1% | 87.5% |
| 1482 | 10.3% | 64.5% | 50.0% | 41.6% | 72.0% | 8.1% | 66.0% | 26.9% | 16.6% | 86.7% | 12.5% | 70.3% | 42.3% | 32.0% | 88.4% |
| 1483 | 8.1% | 65.6% | 48.1% | 30.4% | 73.8% | 6.1% | 52.0% | 35.3% | 35.2% | 87.0% | 9.6% | 66.3% | 43.6% | 29.7% | 87.5% |
| 1484 | 10.5% | 64.8% | 44.6% | 37.9% | 73.8% | 5.8% | 67.5% | 29.1% | 22.0% | 86.7% | 11.1% | 69.9% | 40.2% | 33.0% | 89.1% |
| 1485 | 9.8% | 67.7% | 46.1% | 32.8% | 75.4% | 6.7% | 66.7% | 33.5% | 23.9% | 89.5% | 9.2% | 71.1% | 42.8% | 33.0% | 89.5% |
| 1486 | 12.2% | 59.5% | 37.5% | 32.1% | 69.6% | 6.3% | 56.4% | 25.3% | 18.5% | 87.9% | 11.9% | 64.9% | 34.9% | 28.9% | 88.8% |
| 1487 | 12.2% | 65.7% | 51.0% | 42.7% | 74.0% | 7.7% | 65.0% | 22.7% | 14.0% | 85.4% | 12.5% | 70.7% | 44.8% | 34.1% | 89.2% |
| 1488 | 12.2% | 62.7% | 44.3% | 28.6% | 71.9% | 7.0% | 40.0% | 22.1% | 14.6% | 87.1% | 12.2% | 64.7% | 39.4% | 21.6% | 88.1% |
| 1489 | 13.5% | 59.9% | 40.3% | 34.5% | 70.2% | 5.3% | 61.9% | 27.7% | 25.4% | 86.9% | 12.2% | 63.2% | 37.8% | 31.4% | 85.9% |
| 1490 | 10.1% | 62.2% | 43.0% | 32.5% | 70.2% | 6.4% | 64.9% | 36.4% | 14.0% | 85.5% | 11.2% | 66.4% | 36.8% | 21.7% | 87.2% |
| 1491 | 10.5% | 56.6% | 33.3% | 31.9% | 69.0% | 5.5% | 42.3% | 19.2% | 16.7% | 86.6% | 10.5% | 54.0% | 27.3% | 28.1% | 86.1% |
| 1492 | 13.8% | 59.9% | 41.5% | 35.6% | 68.5% | 7.3% | 61.8% | 18.3% | 15.1% | 85.7% | 15.2% | 68.4% | 32.8% | 24.8% | 88.1% |
| 1493 | 10.3% | 63.1% | 42.6% | 26.8% | 72.1% | 5.4% | 63.4% | 33.0% | 18.2% | 88.9% | 10.7% | 68.6% | 41.4% | 19.7% | 89.2% |
| 1494 | 8.3% | 73.0% | 54.2% | 39.9% | 79.2% | 4.7% | 57.1% | 17.8% | 18.9% | 89.1% | 8.5% | 67.4% | 32.7% | 27.8% | 90.7% |
| 1495 | 12.2% | 57.5% | 37.4% | 36.1% | 71.1% | 7.8% | 42.9% | 20.7% | 22.4% | 85.9% | 12.0% | 52.9% | 29.2% | 32.8% | 88.3% |
| 1496 | 11.4% | 62.4% | 42.8% | 34.3% | 70.3% | 8.9% | 72.2% | 48.6% | 48.5% | 88.3% | 12.6% | 72.4% | 45.3% | 40.1% | 88.9% |
| 1497 | 12.3% | 60.2% | 44.5% | 31.3% | 70.6% | 6.9% | 35.7% | 25.6% | 18.4% | 87.1% | 11.4% | 62.9% | 45.6% | 26.5% | 89.7% |
| 1498 | 10.8% | 60.6% | 41.9% | 37.4% | 69.2% | 5.1% | 40.5% | 14.0% | 17.0% | 86.4% | 9.6% | 57.4% | 27.0% | 28.9% | 85.0% |
| 1499 | 10.5% | 65.5% | 45.7% | 32.0% | 74.1% | 5.0% | 73.3% | 44.0% | 13.5% | 89.4% | 9.4% | 73.4% | 38.5% | 24.1% | 89.5% |
| 1500 | 13.6% | 59.0% | 34.0% | 27.5% | 68.6% | 7.3% | 42.6% | 17.8% | 16.3% | 84.5% | 13.2% | 52.8% | 23.5% | 21.9% | 85.5% |
| 1501 | 11.0% | 63.6% | 46.1% | 40.8% | 70.8% | 8.3% | 63.1% | 18.0% | 13.6% | 85.7% | 12.4% | 70.8% | 25.8% | 20.9% | 88.5% |
| 1502 | 11.3% | 59.7% | 42.7% | 25.8% | 69.1% | 6.0% | 40.3% | 25.8% | 16.8% | 86.5% | 10.2% | 55.9% | 36.1% | 19.0% | 86.1% |
| 1503 | 10.4% | 57.0% | 33.0% | 31.4% | 68.6% | 4.7% | 66.5% | 24.1% | 23.8% | 86.8% | 9.6% | 67.8% | 29.6% | 30.2% | 89.9% |
| 1504 | 10.2% | 58.9% | 37.1% | 25.5% | 68.2% | 5.5% | 55.0% | 21.2% | 14.3% | 84.4% | 11.1% | 60.5% | 26.0% | 19.7% | 86.0% |
| 1505 | 12.2% | 60.4% | 28.8% | 29.6% | 71.0% | 6.4% | 40.6% | 19.0% | 15.5% | 87.8% | 11.6% | 53.0% | 22.7% | 21.3% | 87.0% |
| 1506 | 10.3% | 67.5% | 48.0% | 42.4% | 74.3% | 10.2% | 68.8% | 25.0% | 15.2% | 87.7% | 12.6% | 69.5% | 26.9% | 21.2% | 87.9% |
| 1507 | 13.0% | 58.8% | 34.7% | 17.8% | 69.0% | 6.4% | 33.9% | 20.9% | 17.6% | 88.9% | 12.9% | 52.8% | 27.4% | 17.3% | 84.6% |
| 1508 | 10.7% | 63.8% | 37.6% | 36.9% | 73.3% | 5.0% | 56.5% | 22.1% | 30.8% | 89.9% | 10.2% | 62.3% | 25.9% | 31.2% | 89.4% |
| 1509 | 11.3% | 61.5% | 36.1% | 26.1% | 70.1% | 6.4% | 48.3% | 17.9% | 12.7% | 86.7% | 12.3% | 56.0% | 24.5% | 17.5% | 85.7% |
| 1510 | 10.0% | 63.3% | 29.5% | 33.6% | 76.2% | 4.0% | 58.5% | 26.0% | 17.6% | 90.8% | 8.8% | 63.6% | 24.0% | 22.8% | 91.1% |
| 1511 | 11.4% | 64.2% | 41.7% | 32.3% | 73.2% | 14.2% | 79.0% | 52.7% | 39.5% | 92.0% | 13.5% | 76.3% | 40.4% | 29.0% | 91.0% |
| 1512 | 12.1% | 61.1% | 40.9% | 20.3% | 70.3% | 5.5% | 55.7% | 30.5% | 16.8% | 87.5% | 12.5% | 60.7% | 35.1% | 18.7% | 87.4% |
| 1513 | 9.9% | 64.5% | 38.9% | 35.7% | 72.7% | 3.7% | 52.3% | 15.8% | 25.3% | 91.2% | 8.6% | 61.9% | 23.3% | 30.2% | 89.4% |
| 1514 | 11.2% | 62.9% | 37.9% | 24.1% | 71.9% | 5.6% | 60.5% | 19.3% | 14.0% | 90.0% | 8.4% | 64.1% | 25.0% | 17.7% | 89.7% |
| 1515 | 14.0% | 62.0% | 31.2% | 22.4% | 70.2% | 7.3% | 46.7% | 21.5% | 15.9% | 86.6% | 14.6% | 60.1% | 28.3% | 20.4% | 86.0% |
| 1516 | 11.7% | 60.6% | 38.2% | 30.7% | 69.1% | 10.8% | 66.6% | 24.9% | 17.3% | 87.3% | 12.3% | 64.5% | 26.5% | 19.6% | 86.3% |
| 1517 | 9.9% | 66.1% | 43.0% | 19.9% | 75.2% | 5.9% | 33.9% | 26.0% | 15.9% | 89.6% | 10.6% | 60.8% | 33.9% | 15.8% | 88.2% |
| 1518 | 12.0% | 60.2% | 34.1% | 29.1% | 69.8% | 4.2% | 36.9% | 15.6% | 20.5% | 87.5% | 11.4% | 62.0% | 25.8% | 24.8% | 87.0% |
| 1519 | 12.1% | 62.4% | 36.1% | 24.2% | 70.6% | 6.7% | 49.4% | 18.4% | 13.8% | 87.4% | 11.0% | 59.2% | 24.5% | 18.2% | 88.0% |
| 1520 | 13.8% | 60.5% | 29.7% | 24.5% | 71.7% | 5.9% | 53.2% | 25.8% | 23.2% | 86.5% | 13.1% | 60.5% | 29.6% | 22.8% | 88.2% |
| 1521 | 11.2% | 63.8% | 42.0% | 34.1% | 70.2% | 6.9% | 67.8% | 28.1% | 14.5% | 85.8% | 13.4% | 69.0% | 31.0% | 20.1% | 86.3% |
| 1522 | 11.9% | 58.4% | 35.1% | 18.5% | 68.6% | 7.8% | 66.1% | 49.0% | 32.2% | 89.4% | 10.2% | 67.9% | 46.4% | 27.7% | 89.3% |
| 1523 | 10.7% | 65.6% | 38.7% | 24.4% | 73.5% | 5.5% | 64.3% | 33.5% | 20.5% | 88.1% | 10.4% | 67.4% | 31.8% | 21.6% | 88.4% |
| 1524 | 3.0% | 68.2% | 17.7% | 15.7% | 85.2% | 3.9% | 48.4% | 17.8% | 12.0% | 93.9% | 3.7% | 42.9% | 17.0% | 12.2% | 95.9% |
| 1525 | 2.8% | 7.7% | 4.8% | 3.2% | 34.6% | 1.8% | 8.8% | 11.3% | 22.4% | 83.2% | 2.3% | 7.1% | 9.2% | 16.6% | 84.1% |
| 1526 | 1.4% | 4.8% | 2.3% | 1.8% | 17.9% | 0.7% | 6.4% | 19.6% | 4.1% | 80.9% | 1.0% | 6.4% | 17.0% | 3.2% | 77.8% |
| 1527 | 1.9% | 5.0% | 2.8% | 1.9% | 18.0% | 1.2% | 6.5% | 3.8% | 3.1% | 29.6% | 1.4% | 4.7% | 2.7% | 1.9% | 28.1% |
| 1528 | 2.5% | 5.9% | 3.3% | 2.3% | 15.5% | 2.3% | 9.6% | 5.6% | 4.2% | 34.7% | 3.3% | 7.7% | 4.1% | 2.9% | 32.3% |
| 1529 | 2.2% | 5.3% | 2.8% | 2.0% | 19.4% | 1.7% | 9.1% | 4.5% | 3.5% | 22.8% | 2.8% | 7.7% | 3.1% | 2.1% | 24.8% |
| 1530 | 12.3% | 30.6% | 17.6% | 11.4% | 39.2% | 7.0% | 43.0% | 23.8% | 16.8% | 72.2% | 15.4% | 49.7% | 25.1% | 15.4% | 72.1% |
| 1531 | 8.4% | 7.7% | 9.8% | 7.9% | 85.0% | 1.6% | 7.0% | 20.0% | 4.2% | 94.9% | 2.3% | 6.7% | 18.0% | 4.2% | 96.7% |
| 1532 | 5.5% | 11.0% | 5.5% | 4.0% | 44.4% | 2.6% | 25.8% | 6.2% | 4.9% | 76.9% | 3.5% | 19.1% | 4.9% | 2.9% | 75.4% |
| 1533 | 2.4% | 5.2% | 2.9% | 1.8% | 18.7% | 1.1% | 6.3% | 3.8% | 2.9% | 42.9% | 1.8% | 4.5% | 2.3% | 1.8% | 40.2% |
| 1534 | 4.3% | 14.8% | 5.8% | 3.8% | 41.8% | 2.7% | 11.9% | 6.7% | 5.3% | 74.1% | 2.9% | 11.4% | 4.5% | 3.2% | 72.0% |
| 1535 | 5.2% | 12.7% | 5.7% | 3.6% | 22.2% | 6.5% | 15.3% | 6.6% | 4.5% | 51.7% | 8.5% | 21.1% | 5.8% | 3.1% | 55.5% |
| 1536 | 3.4% | 10.7% | 5.4% | 3.3% | 28.5% | 1.9% | 15.4% | 6.2% | 4.2% | 51.6% | 3.3% | 18.4% | 6.8% | 3.8% | 55.1% |
| 1537 | 2.7% | 9.8% | 4.7% | 3.0% | 22.3% | 1.9% | 9.8% | 5.4% | 4.1% | 30.9% | 2.2% | 11.9% | 4.6% | 2.9% | 32.2% |
| 1538 | 8.1% | 81.4% | 25.7% | 5.6% | 89.7% | 4.6% | 25.2% | 35.2% | 53.2% | 90.0% | 5.4% | 30.9% | 35.1% | 51.6% | 93.0% |
| 1539 | 9.3% | 77.5% | 24.0% | 9.5% | 87.1% | 4.0% | 15.1% | 10.8% | 6.0% | 73.0% | 5.4% | 21.9% | 10.8% | 5.2% | 75.9% |
| 1540 | 5.8% | 15.5% | 4.8% | 2.4% | 31.0% | 1.3% | 9.1% | 5.6% | 2.2% | 87.8% | 2.0% | 10.0% | 4.9% | 1.7% | 90.0% |
| 1541 | 4.7% | 24.8% | 7.1% | 4.5% | 51.2% | 2.5% | 50.8% | 8.3% | 3.9% | 81.0% | 4.2% | 56.6% | 8.1% | 3.5% | 83.3% |
| 1542 | 5.3% | 19.1% | 9.3% | 5.6% | 35.1% | 2.9% | 24.1% | 9.3% | 6.5% | 74.1% | 3.1% | 26.3% | 9.0% | 5.2% | 71.7% |
| 1543 | 3.3% | 14.1% | 7.0% | 4.4% | 25.4% | 1.8% | 13.3% | 5.6% | 4.3% | 51.6% | 2.4% | 18.7% | 7.7% | 4.5% | 54.5% |
| 1544 | 5.2% | 24.4% | 15.4% | 9.0% | 57.0% | 3.0% | 21.2% | 12.5% | 11.6% | 71.3% | 4.5% | 24.2% | 16.2% | 13.2% | 73.0% |
| 1545 | 23.1% | 88.2% | 36.4% | 26.7% | 94.1% | 11.0% | 92.1% | 82.3% | 70.4% | 97.7% | 10.5% | 92.5% | 76.7% | 61.8% | 98.6% |
| 1546 | 10.6% | 69.3% | 22.3% | 12.8% | 80.6% | 4.9% | 74.3% | 32.9% | 26.5% | 93.9% | 5.7% | 78.5% | 35.1% | 31.9% | 96.1% |
| 1547 | 8.4% | 69.4% | 31.2% | 17.2% | 77.9% | 4.8% | 55.8% | 35.7% | 39.6% | 90.6% | 6.5% | 72.5% | 40.9% | 37.4% | 93.6% |
| 1548 | 3.9% | 24.9% | 9.9% | 8.6% | 53.4% | 2.0% | 20.5% | 6.1% | 12.7% | 93.3% | 2.5% | 30.2% | 10.5% | 37.6% | 95.6% |
| 1549 | 4.9% | 28.5% | 19.3% | 10.9% | 76.1% | 3.1% | 16.3% | 9.8% | 7.6% | 90.1% | 4.1% | 15.8% | 9.1% | 6.8% | 90.6% |
| 1550 | 5.3% | 17.8% | 12.6% | 10.8% | 76.9% | 2.8% | 17.7% | 8.2% | 13.2% | 92.4% | 3.3% | 14.7% | 7.8% | 11.4% | 94.8% |
| 1551 | 4.8% | 13.2% | 8.4% | 6.2% | 56.6% | 2.8% | 14.0% | 9.7% | 7.5% | 63.0% | 3.3% | 12.1% | 8.8% | 5.9% | 72.2% |
| 1552 | 5.3% | 80.6% | 41.2% | 33.7% | 87.0% | 3.9% | 48.1% | 32.5% | 57.0% | 91.7% | 5.4% | 69.2% | 46.0% | 53.5% | 94.4% |
| 1553 | 3.0% | 40.5% | 26.1% | 19.6% | 82.3% | 2.7% | 13.1% | 7.4% | 9.7% | 95.0% | 2.7% | 19.8% | 12.5% | 13.4% | 97.5% |
| 1554 | 3.9% | 50.9% | 48.1% | 43.6% | 88.3% | 3.5% | 27.2% | 8.3% | 49.6% | 94.8% | 3.6% | 48.5% | 21.4% | 44.1% | 96.3% |
| 1555 | 4.9% | 38.0% | 26.8% | 23.9% | 84.2% | 3.6% | 30.3% | 12.6% | 11.4% | 83.4% | 5.6% | 32.9% | 14.4% | 14.5% | 87.3% |
| 1556 | 7.1% | 19.4% | 15.5% | 8.5% | 79.3% | 4.0% | 24.0% | 13.6% | 9.8% | 68.4% | 5.5% | 20.7% | 12.2% | 8.1% | 78.7% |
| 1557 | 4.4% | 10.4% | 10.7% | 6.2% | 84.7% | 3.7% | 18.2% | 11.2% | 18.7% | 92.5% | 3.5% | 15.4% | 10.6% | 24.3% | 95.9% |
| 1558 | 2.8% | 8.2% | 4.4% | 3.2% | 59.4% | 1.8% | 10.3% | 6.5% | 5.8% | 66.4% | 2.0% | 7.8% | 4.3% | 3.5% | 60.3% |
| 1559 | 8.6% | 25.8% | 35.6% | 35.0% | 80.1% | 5.2% | 25.8% | 17.7% | 27.0% | 90.5% | 8.3% | 24.7% | 24.8% | 31.6% | 91.5% |
| 1560 | 6.4% | 12.5% | 8.2% | 43.9% | 88.0% | 2.4% | 15.3% | 8.2% | 14.3% | 93.0% | 4.3% | 14.3% | 7.0% | 18.4% | 94.5% |
| 1561 | 3.5% | 8.5% | 6.5% | 11.5% | 86.2% | 2.4% | 28.7% | 15.7% | 18.7% | 88.6% | 3.3% | 17.7% | 12.6% | 12.8% | 90.7% |
| 1562 | 6.3% | 16.9% | 9.8% | 7.5% | 73.2% | 4.8% | 25.6% | 15.1% | 36.1% | 82.2% | 6.8% | 22.5% | 12.8% | 18.8% | 75.9% |
| 1563 | 19.7% | 23.9% | 13.5% | 9.7% | 62.0% | 6.1% | 30.7% | 18.3% | 13.8% | 83.9% | 13.4% | 30.6% | 15.8% | 12.0% | 82.1% |
| 1564 | 11.7% | 31.2% | 19.0% | 13.5% | 65.0% | 7.4% | 35.9% | 20.8% | 17.0% | 68.6% | 11.8% | 35.6% | 19.5% | 15.0% | 72.3% |
| 1565 | 9.3% | 26.9% | 15.7% | 10.6% | 58.8% | 5.4% | 30.3% | 17.3% | 13.8% | 65.2% | 9.8% | 33.2% | 17.1% | 13.1% | 63.5% |
| 1566 | 57.5% | 80.8% | 71.4% | 62.0% | 84.4% | 8.9% | 81.8% | 21.9% | 14.6% | 91.4% | 18.3% | 87.2% | 61.5% | 46.1% | 94.0% |
| 1567 | 60.6% | 84.0% | 74.8% | 62.6% | 86.7% | 10.2% | 85.9% | 22.9% | 15.7% | 93.4% | 15.8% | 91.2% | 61.1% | 44.6% | 95.8% |
| 1568 | 48.6% | 78.1% | 60.3% | 55.4% | 82.6% | 11.6% | 83.3% | 26.1% | 17.4% | 91.9% | 15.7% | 88.1% | 35.4% | 30.0% | 94.6% |
| 1569 | 60.8% | 87.9% | 74.2% | 66.2% | 90.2% | 12.5% | 88.7% | 28.0% | 20.9% | 94.4% | 14.9% | 93.4% | 39.0% | 34.9% | 96.9% |
| 1570 | 57.6% | 84.0% | 72.2% | 63.7% | 86.7% | 11.5% | 85.6% | 26.9% | 17.6% | 93.2% | 15.6% | 91.0% | 47.2% | 37.4% | 95.6% |
| 1571 | 59.3% | 83.1% | 70.1% | 61.7% | 86.1% | 11.3% | 83.3% | 25.7% | 17.5% | 92.2% | 16.6% | 88.3% | 39.4% | 33.5% | 94.5% |
| 1572 | 56.1% | 84.5% | 71.3% | 63.0% | 87.5% | 5.8% | 80.4% | 17.2% | 11.6% | 91.7% | 10.8% | 89.4% | 32.6% | 23.5% | 95.1% |
| 1573 | 63.2% | 90.8% | 75.5% | 69.6% | 92.5% | 13.5% | 92.2% | 29.7% | 20.7% | 96.3% | 18.3% | 95.3% | 38.8% | 35.7% | 97.9% |
| 1574 | 58.7% | 83.8% | 69.8% | 63.9% | 86.7% | 7.8% | 84.1% | 20.7% | 14.7% | 93.1% | 14.0% | 89.9% | 39.0% | 31.5% | 95.5% |
| 1575 | 65.3% | 88.2% | 76.9% | 69.9% | 90.4% | 8.8% | 89.8% | 22.7% | 15.8% | 95.6% | 13.1% | 94.2% | 48.4% | 38.4% | 97.5% |
| 1576 | 46.2% | 74.7% | 56.7% | 50.6% | 79.4% | 9.7% | 77.3% | 25.5% | 16.9% | 89.3% | 15.3% | 84.6% | 31.7% | 27.0% | 93.0% |
| 1577 | 50.1% | 76.4% | 61.2% | 53.0% | 81.2% | 10.0% | 82.8% | 23.9% | 15.7% | 92.5% | 15.8% | 87.3% | 46.6% | 38.2% | 94.4% |
| 1578 | 50.8% | 76.6% | 65.0% | 58.0% | 80.6% | 9.3% | 77.5% | 22.8% | 17.6% | 89.3% | 18.1% | 81.7% | 51.1% | 40.8% | 91.2% |
| 1579 | 47.1% | 76.5% | 58.2% | 52.4% | 80.7% | 9.9% | 78.7% | 24.7% | 15.8% | 91.0% | 16.8% | 83.5% | 43.5% | 34.8% | 92.4% |
| 1580 | 50.6% | 77.7% | 63.4% | 55.9% | 81.7% | 9.5% | 83.4% | 25.0% | 16.9% | 93.7% | 17.5% | 86.3% | 50.8% | 39.0% | 94.1% |
| 1581 | 52.6% | 81.6% | 65.0% | 58.1% | 85.0% | 11.9% | 82.1% | 25.8% | 17.2% | 92.0% | 15.9% | 89.2% | 35.9% | 30.9% | 95.2% |
| 1582 | 49.2% | 73.9% | 62.1% | 56.1% | 78.5% | 10.3% | 75.2% | 21.2% | 13.9% | 88.4% | 17.6% | 78.7% | 49.7% | 39.0% | 89.6% |
| 1583 | 43.0% | 75.7% | 58.1% | 50.7% | 80.5% | 10.0% | 80.3% | 25.3% | 16.8% | 91.9% | 17.4% | 83.8% | 47.8% | 36.9% | 93.2% |
| 1584 | 54.0% | 84.5% | 67.2% | 58.3% | 87.3% | 11.7% | 84.0% | 26.9% | 17.3% | 92.8% | 15.6% | 89.8% | 39.5% | 30.8% | 95.4% |
| 1585 | 53.0% | 82.4% | 65.5% | 57.4% | 86.1% | 8.4% | 81.4% | 26.1% | 18.2% | 91.7% | 15.7% | 87.9% | 39.4% | 32.3% | 94.9% |
| 1586 | 51.6% | 77.2% | 66.5% | 59.8% | 81.5% | 10.0% | 82.7% | 24.2% | 16.4% | 92.4% | 17.4% | 85.2% | 55.3% | 44.5% | 93.4% |
| 1587 | 55.3% | 83.8% | 70.4% | 62.3% | 87.3% | 10.8% | 87.0% | 25.8% | 17.9% | 94.8% | 14.6% | 89.2% | 55.6% | 42.9% | 95.5% |
| 1588 | 54.8% | 88.5% | 67.1% | 61.4% | 90.6% | 8.9% | 85.6% | 28.4% | 22.3% | 93.3% | 14.3% | 91.3% | 31.4% | 26.8% | 96.2% |
| 1589 | 38.7% | 74.6% | 47.2% | 39.5% | 79.7% | 9.8% | 70.6% | 25.9% | 17.5% | 86.5% | 17.7% | 78.5% | 33.3% | 25.6% | 89.7% |

### Example 10

### Selected Engineered Guide RNA Compositions Targeting DUX4

This example describes the top 50 engineered guide RNAs that target the polyadenylation (polyA) signal site (ATTAAA) in the “pLAM” region of DUX4 mRNA. The corresponding positions for each “A” in the polyA signal site sequence (ATTAAA) are denoted as 0, 3, 4, and 5. Each of these positions was targeted for editing using different engineered guide RNA sequences and the top 50 engineered guide RNAs for editing were identified. The RNA sequence for the polyA signal site is (AUUAAA). Self-annealing RNA structures, which comprised (i) the engineered guide RNAs shown in TABLE 3 and (ii) the RNA sequences of the DUX4 region targeted by the engineered guide RNAs, were contacted with ADAR1 for 30 minutes under conditions that allowed for editing. The regions targeted by the engineered guide RNAs were subsequently assessed for editing using next generation sequencing (NGS). All polynucleotide sequences encoding for the engineered guide RNAs of TABLE 3, are encompassed herein, which are represented by each of the SEQ ID NOs shown in TABLE 3, with a T substituted for each U. For each sequence, the structural features formed in the double stranded RNA substrate upon hybridization of the guide RNA to the target DUX4 RNA, are shown in the second column of TABLE 3. For reference, each structural feature formed within a guide-target RNA scaffold (target RNA sequence hybridized to an engineered guide RNA) is annotated as follows:
a. the position of the structural feature with respect to the target A (position 0) of the target RNA sequence, with a negative value indicating upstream (5′) of the target A and a positive value indicating downstream (3′) of the target A;
b. the number of bases in the target RNA sequence and the number of bases in the engineered guide RNA that together form the structural feature—for example, 6/6 indicates that six contiguous bases from the target RNA sequence and six contiguous bases from the engineered guide RNA form the structural feature;
c. the name of the structural feature (e.g., symmetric bulge, symmetric internal loop, asymmetric bulge, asymmetric internal loop, mismatch, or wobble base pair), and
d. the sequences of bases on the target RNA side and the engineered guide RNA side that participate in forming the structural feature.

For example, with reference to SEQ ID NO: 8, “20_6-6_internal_loop-symmetric_UGGAUC-ACAGGU” is read as a structural feature formed in a guide-target RNA scaffold (target DUX4 RNA sequence hybridized to an engineered guide RNA of SEQ ID NO: 8), where
a. the structural feature starts 20 nucleotides downstream (3′) (the +20 position) from the target A (0 position) of the target RNA sequence
b. six contiguous bases from the target RNA sequence and six contiguous bases from the engineered guide RNA form the structural feature
c. the structural feature is an internal symmetric loop
d. a sequence of UGGAUC from the target RNA side and a sequence of ACAGGU from the engineered guide RNA side participate in forming the internal symmetric loop.

TABLE 3
Top 50 engineered guide RNAs that target the polyadenylation (polyA)
signal site (ATTAAA) in the “pLAM” region of DUX4.

| | |
|---|---|
| Guide | |
| Seq ID | |
| No: | Structural Features |

Targeting Position 0

| | |
|---|---|
| 1575 | -6_6-6 internal_loop-symmetric_UUCAGA-GGAACU; 3_1-1_mismatch_A- |
| | C; 33 6-6_internal_loop-symmetric_AGAUUU-CUGACC; 49_3-3 bulge- |
| | symmetric_UGU-CUU; 62_3-3_bulge-symmetric_GAG-GAG; 75_3-3_bulge- |
| | symmetricAUA-GUG |
| 593 | -9_6-6_internal_loop-symmetric_UAGUUC-CGUGAU; 0_1-1_mismatch_A- |
| | C; 40_6-6_internal_loop-symmetric_CAUCUU-CCCUCC |
| 1573 | -6_6-6_internal_loop-symmetric_UUCAGA-GGAACU; 3_1-1_mismatch_A- |
| | C; 33_6-6_internal_loop-symmetric_AGAUUU-CUGACC; 47_5- |
| | 5_internal_loop-symmetric_UGUGU-UGAAU; 60_5-5_internal_loop- |
| | symmetric_CAGAG-GCACC; 73_5-5_internal_loop-symmetric_CAAUA- |
| | AAGUC |
| 934 | -6_6-6_internal_loop-symmetric_UUCAGA-CACCUC; 0_1-1_mismatch_A-C; |
| | 33_6-6_internal_loop-symmetric_AGAUUU-UCCCUA |
| 1569 | -6_6-6 internal_loop-symmetric_UUCAGA-GGAACU; 3_1-1_mismatch_A- |
| | C; 33_6-6_internal_loop-symmetric_AGAUUU-CUGACC; 45_5- |
| | 5_internal_loop-symmetric_UUUGU-UGUUC; 56_5-5_internal_loop- |
| | symmetric_AGUGC-CUUUC; 67_5-5_internal_loop-symmetric_AUGUC- |
| | CCAAA |
| 1567 | -6_6-6_internal_loop-symmetric_UUCAGA-GGAACU; 3_1-1_mismatch_A- |
| | C; 33_6-6_internal_loop-symmetric_AGAUUU-CUGACC; 45_3-3_bulge- |
| | symmetric_UUU-UAU; 54_3-3_bulge-symmetric_UGA-CUU; 63_3-3_bulge- |
| | symmetric_AGA-GAC; 72_3-3_bulge-symmetric_ACA-GCA |
| 851 | -7_6-6_internal_loop-symmetric_GUUCAG-ACGUCG; 0_1-1_mismatch_A-C; |
| | 42_6-6_internal_loop-symmetric_UCUUUU-CCGCUC |
| 1211 | -4_6-6_internal_loop-symmetric_CAGAGA-ACAGGC; 0_1-1_mismatch_A-C; |
| | 42_6-6_internal_loop-symmetric_UCUUUU-CCCCUC |
| 1571 | -6_6-6_internal_loop-symmetric_UUCAGA-GGAACU; 3_1-1_mismatch_A- |
| | C; 33_6-6_internal_loop-symmetric_AGAUUU-CUGACC; 47_3-3_bulge- |
| | symmetric_UGU-UGU; 58_3-3_bulge-symmetric_UGC-AAU; 69_3-3_bulge- |
| | symmetric_GUC-CCA |
| 937 | -6_6-6_internal_loop-symmetric_UUCAGA-AUAAGU; 0_1-1_mismatch_A- |
| | C; 40_6-6_internal_loop-symmetric_CAUCUU-UGCUCC |
| 1574 | -6_6-6_internal_loop-symmetric_UUCAGA-GGAACU; 3_1-1_mismatch_A- |
| | C; 33_6-6_internal_loop-symmetric_AGAUUU-CUGACC; 49_2-2_bulge- |
| | symmetric_UG-GC; 61_2-2_bulge-symmetric_AG-AA; 73_2-2_bulge- |
| | symmetric_CA-AC |
| 1570 | -6_6-6_internal_loop-symmetric_UUCAGA-GGAACU; 3_1-1_mismatch_A- |
| | C; 33_6-6_internal_loop-symmetric_AGAUUU-CUGACC; 47_2-2_bulge- |
| | symmetric_UG-AU; 57_2-2_bulge-symmetric_GU-CG; 67_2-2_bulge- |
| | symmetric_AU-UA; 77_2-2_bulge-symmetric_AU-UG |
| 1566 | -6_6-6_internal_loop-symmetric_UUCAGA-GGAACU; 3_1-1_mismatch_A- |
| | C; 33_6-6_internal_loop-symmetric_AGAUUU-CUGACC; 45_2-2_bulge- |
| | symmetric_UU-CC; 53_2-2_bulge-symmetric_AU-CG; 61_2-2_bulge- |
| | symmetric_AG-GA; 69_2-2_bulge-symmetric_GU-UG; 77_2-2_bulge- |
| | symmetric_AU-CC |
| 1117 | -4_6-6_internal_loop-symmetric_CAGAGA-GUAAUC; 0_1-1_mismatch_A- |
| | C; 23_6-6_internal_loop-symmetric_AUCCUA-CCUUUC |
| 906 | -6_6-6_internal_loop-symmetric_UUCAGA-AGCUCC; 0_1-1_mismatch_A-C; |
| | 27_6-6_internal_loop-symmetric_UAUAGA-GUGGGC |
| 1572 | -6_6-6_internal_loop-symmetric_UUCAGA-GGAACU; 3_1-1_mismatch_A- |
| | C; 33_6-6_internal_loop-symmetric_AGAUUU-CUGACC; 47_4-4_bulge- |
| | symmetric_UGUG-ACCU; 59_4-4_bulge-symmetric_GCAG-GGUG; 71_4- |
| | 4_bulge-symmetric_CACA-CUUU |
| 1104 | -5_6-6_internal_loop-symmetric_UCAGAG-GGGUCC; 3_1-1_mismatch A-C; |
| | 44_6-6_internal_loop-symmetric_UUUUGU-UGCCCC |
| 352 | -11_6-6_internal_loop-symmetric_AUUAGU-UAAGUC; 0_1-1_mismatch_A- |
| | C; 41_6-6_internal_loop-symmetric_AUCUUU-UUCCCC |
| 512 | -9_6-6_internal_loop-symmetric_UAGUUC-AUCAUU; 0_1-1_mismatch_A- |
| | C; 22_6-6_internal_loop-symmetric_GAUCCU-UCUCCG |
| 1587 | -6_6-6_internal_loop-symmetric_UUCAGA-GGAACU; 3_1-1_mismatch_A- |
| | C; 33_6-6_internal_loop-symmetric_AGAUUU-CUGACC; 55_3-3_bulge- |
| | symmetric_GAG-GCG; 74_3-3_bulge-symmetric_AAU-UCA |
| 375 | -10_6-6_internal_loop-symmetric_UUAGUU-UUGAUU; 0_1-1_mismatch_A- |
| | C; 20_6-6_internal_loop-symmetric_UGGAUC-UGCGGU |
| 1588 | -6_6-6_internal_loop-symmetric_UUCAGA-GGAACU; 3_1-1_mismatch_A- |
| | C; 33_6-6_internal_loop-symmetric_AGAUUU-CUGACC; 55_4-4_bulge- |
| | symmetric_GAGU-CAAA; 75_4-4_bulge-symmetric_AUAU-CGCG |
| 977 | -6_6-6_internal_loop-symmetric_UUCAGA-AGCAUC; 3_1-1_mismatch_A-C; |
| | 42_6-6_internal_loop-symmetric_UCUUUU-CUGUCC |
| 642 | -8_6-6_internal_loop-symmetric_AGUUCA-AUUGUA; 0_1-1_mismatch_A- |
| | C; 24_6-6 internal_loop-symmetric_UCCUAU-CCUUUU |
| 1236 | -3_6-6_internal_loop-symmetric_AGAGAU-UAGAAA; 0_1-1_mismatch_A- |
| | C; 24_6-6_internal_loop-symmetric_UCCUAU-CCUUCU |
| 1584 | -6_6-6_internal_loop-symmetric_UUCAGA-GGAACU; 3_1-1_mismatch_A- |
| | C; 33_6-6_internal_loop-symmetric_AGAUUU-CUGACC; 53_4-4_bulge- |
| | symmetric_AUGA-AGCC; 71_4-4_bulge-symmetric_CACA-AUAA |
| 252 | -11_6-6_internal_loop-symmetric_AUUAGU-UAAUCC; 0_1-1 mismatch_A- |
| | C; 20_6-6_internal_loop-symmetric_UGGAUC-AGCGGU |
| 394 | -10_6-6_internal_loop-symmetric_UUAGUU-UUACAC; 0_1-1_mismatch_A- |
| | C; 24_6-6_internal_loop-symmetric_UCCUAU-UCAUCC |
| 482 | -10_6-6_internal_loop-symmetric_UUAGUU-UUACAU; 0_1-1 mismatch_A- |
| | C; 42_6-6_internal_loop-symmetric_UCUUUU-UACGCC |
| 1585 | -6_6-6_internal_loop-symmetric_UUCAGA-GGAACU; 3_1-1_mismatch_A- |
| | C; 33_6-6_internal_loop-symmetric_AGAUUU-CUGACC; 53_5- |
| | 5_internal_loop-symmetric_AUGAG-GAAAA; 72_5-5_internal_loop- |
| | symmetric_ACAAU-UCAAG |
| 291 | -11_6-6_internal_loop-symmetric_AUUAGU-UGAGCA; 0_1-1_mismatch_A- |
| | C; 28_6-6_internal_loop-symmetric_AUAGAA-CCUCCA |
| 356 | -11_6-6_internal_loop-symmetric_AUUAGU-CGACCG; 0_1-1_mismatch_A- |
| | C; 42_6-6_internal_loop-symmetric_UCUUUU-UCCUUU |
| 1054 | -5_6-6_internal_loop-symmetric_UCAGAG-GAGAUC; 33_6-6_internal_loop- |
| | symmetric_AGAUUU-CCUGGA |
| 1581 | -6_6-6_internal_loop-symmetric_UUCAGA-GGAACU; 3_1-1_mismatch_A- |
| | C; 33_6-6_internal_loop-symmetric_AGAUUU-CUGACC; 51_5- |
| | 5_internal_loop-symmetric_UGAUG-GUAGU; 68_5-5_internal_loop- |
| | symmetric_UGUCA-GCAGU |
| 1103 | -5_6-6_internal_loop-symmetric_UCAGAG-GCAGCU; 0_1-1_mismatch_A-C; |
| | 44_6-6 internal_loop-symmetric_UUUUGU-CUACUC |
| 502 | -9_6-6_internal_loop-symmetric_UAGUUC-UUUUGC; 0_1-1_mismatch_A- |
| | C; 20_6-6_internal_loop-symmetric_UGGAUC-ACUAUU |
| 769 | -7 6-6_internal_loop-symmetric_GUUCAG-AAAAUA; 0_1-1_mismatch_A- |
| | C; 25_6-6_internal_loop-symmetric_CCUAUA-GACUCC |
| 408 | -10_6-6_internal_loop-symmetric_UUAGUU-CCUCGC; 0_1-1_mismatch_A- |
| | C; 27_6-6_internal_loop-symmetric_UAUAGA-AACCCC |
| 1586 | -6_6-6_internal_loop-symmetric_UUCAGA-GGAACU; 3_1-1_mismatch_A- |
| | C; 33_6-6_internal_loop-symmetric_AGAUUU-CUGACC; 55_2-2_bulge- |
| | symmetric_GA-AA; 73_2-2_bulge-symmetric_CA-CC |
| 1008 | -5_6-6_internal_loop-symmetric_UCAGAG-GAGAGU; 0_1-1_mismatch_A- |
| | C; 24_6-6_internal_loop-symmetric_UCCUAU-UUUUCU |
| 737 | -8_6-6_internal_loop-symmetric_AGUUCA-GGUUUC; 0_1-1_mismatch_A- |
| | C; 44_6-6_internal_loop-symmetric_UUUUGU-UGCCGU |
| 985 | -6_6-6_internal_loop-symmetric_UUCAGA-GGGCCU; 0_1-1_mismatch_A-C; |
| | 44_6-6_internal_loop-symmetric_UUUUGU-UUGGUC |
| 679 | -8_6-6_internal_loop-symmetric_AGUUCA-ACCUAG; 0_1-1_mismatch_A-C; |
| | 32_6-6_internal_loop-symmetric_AAGAUU-CUAACC |
| 727 | -8_6-6_internal_loop-symmetric_AGUUCA-ACUUCG; 0_1-1_mismatch_A-C; |
| | 42 6-6 internal loop-symmetric_UCUUUU-CUCGAU |
| 1578 | -6_6-6_internal_loop-symmetric_UUCAGA-GGAACU; 3_1-1_mismatch_A- |
| | C; 33_6-6_internal_loop-symmetric_AGAUUU-CUGACC; 51_2-2_bulge- |
| | symmetric_UG-GC; 65_2-2_bulge-symmetric_AU-UG |
| 365 | -11_6-6_internal_loop-symmetric_AUUAGU-CCAAAG; 0_1-1_mismatch_A- |
| | C; 44_6-6_internal_loop-symmetric_UUUUGU-UAACCU |
| 1580 | -6_6-6_internal_loop-symmetric_UUCAGA-GGAACU; 3_1-1 mismatch_A- |
| | C; 33_6-6_internal_loop-symmetric_AGAUUU-CUGACC; 51_4-4_bulge- |
| | symmetric_UGAU-CUAC; 67_4-4_bulge-symmetric_AUGU-UGUC |
| 487 | -10_6-6_internal_loop-symmetric_UUAGUU-CCUCAC; 0_1-1_mismatch_A- |
| | C; 43_6-6_internal_loop-symmetric_CUUUUG-AUCGCU |
| 1098 | -5_6-6_internal_loop-symmetric_UCAGAG-GCGACC; 0_1-1_mismatch_A-C; |
| | 43_6-6_internal_loop-symmetric_CUUUUG-GCACCC |
| 976 | -6_6-6_internal_loop-symmetric_UUCAGA-GUGUUU; 0_1-1_mismatch_A- |
| | C; 42_6-6_internal_loop-symmetric_UCUUUU-CGUCGU |

Targeting Position 3

| | |
|---|---|
| 1573 | -6_6-6_internal_loop-symmetric_UUCAGA-GGAACU; 3_1-1_mismatch_A- |
| | C; 33_6-6_internal_loop-symmetric_AGAUUU-CUGACC; 47_5- |
| | 5_internal_loop-symmetric_UGUGU-UGAAU; 60_5-5_internal_loop- |
| | symmetric_CAGAG-GCACC; 73_5-5_internal_loop-symmetric_CAAUA- |
| | AAGUC |
| 1588 | -6_6-6_internal_loop-symmetric_UUCAGA-GGAACU; 3_1-1_mismatch_A- |
| | C; 33_6-6_internal_loop-symmetric_AGAUUU-CUGACC; 55_4-4_bulge- |
| | symmetric_GAGU-CAAA; 75_4-4_bulge-symmetric_AUAU-CGCG |
| 1545 | -5_2-2_bulge-symmetric_AG-GC; 10_2-2_bulge-symmetric_CC-AU; 26_2- |
| | 2_bulge-symmetric_CU-UU; 42_2-2_bulge-symmetric_UC-CC; 58_2-2_bulge- |
| | symmetric_UG-GU; 74_2-2_bulge-symmetric_AA-AG |
| 1575 | -6_6-6_internal_loop-symmetric_UUCAGA-GGAACU; 3_1-1_mismatch_A- |
| | C; 33_6-6_internal_loop-symmetric_AGAUUU-CUGACC; 49_3-3_bulge- |
| | symmetric_UGU-CUU; 62_3-3_bulge-symmetric_GAG-GAG; 75_3-3_bulge- |
| | symmetric_AUA-GUG |
| 1569 | -6_6-6_internal_loop-symmetric_UUCAGA-GGAACU; 3_1-1_mismatch_A- |
| | C; 33_6-6_internal_loop-symmetric_AGAUUU-CUGACC; 45_5- |
| | 5_internal_loop-symmetric_UUUGU-UGUUC; 56_5-5_internal_loop- |
| | symmetric_AGUGC-CUUUC; 67_5-5_internal_loop-symmetric_AUGUC- |
| | CCAAA |
| 1584 | -6_6-6_internal_loop-symmetric_UUCAGA-GGAACU; 3_1-1_mismatch_A- |
| | C; 33_6-6_internal_loop-symmetric_AGAUUU-CUGACC; 53_4-4_bulge- |
| | symmetric_AUGA-AGCC; 71_4-4_bulge-symmetric_CACA-AUAA |
| 1572 | -6_6-6_internal_loop-symmetric_UUCAGA-GGAACU; 3_1-1_mismatch_A- |
| | C; 33_6-6_internal_loop-symmetric_AGAUUU-CUGACC; 47_4-4_bulge- |
| | symmetric_UGUG-ACCU; 59_4-4_bulge-symmetric_GCAG-GGUG; 71_4- |
| | 4_bulge-symmetric_CACA-CUUU |
| 1567 | -6_6-6_internal_loop-symmetric_UUCAGA-GGAACU; 3_1-1_mismatch_A- |
| | C; 33_6-6_internal_loop-symmetric_AGAUUU-CUGACC; 45_3-3_bulge- |
| | symmetric_UUU-UAU; 54_3-3_bulge-symmetric_UGA-CUU; 63_3-3_bulge- |
| | symmetric_AGA-GAC; 72_3-3 bulge-symmetric_ACA-GCA |
| 1570 | -6_6-6_internal_loop-symmetric_UUCAGA-GGAACU; 3_1-1_mismatch_A- |
| | C; 33_6-6_internal_loop-symmetric_AGAUUU-CUGACC; 47_2-2_bulge- |
| | symmetric_UG-AU; 57_2-2_bulge-symmetric_GU-CG; 67_2-2_bulge- |
| | symmetric_AU-UA; 77_2-2 bulge-symmetric_AU-UG |
| 1587 | -6_6-6_internal_loop-symmetric_UUCAGA-GGAACU; 3_1-1_mismatch_A- |
| | C; 33_6-6_internal_loop-symmetric_AGAUUU-CUGACC; 55_3-3_bulge- |
| | symmetric_GAG-GCG; 74_3-3_bulge-symmetric_AAU-UCA |
| 1574 | -6_6-6_internal_loop-symmetric_UUCAGA-GGAACU; 3_1-1_mismatch_A- |
| | C; 33_6-6_internal_loop-symmetric_AGAUUU-CUGACC; 49_2-2_bulge- |
| | symmetric_UG-GC; 61_2-2_bulge-symmetric_AG-AA; 73_2-2_bulge- |
| | symmetric_CA-AC |
| 625 | -8_6-6_internal_loop-symmetric_AGUUCA-CAGUUC; 5_1-1_mismatch_A-C; |
| | 20_6-6_internal_loop-symmetric_UGGAUC-AAGUGU |
| 1571 | -6_6-6_internal_loop-symmetric_UUCAGA-GGAACU; 3_1-1_mismatch_A- |
| | C; 33_6-6_internal_loop-symmetric_AGAUUU-CUGACC; 47_3-3_bulge- |
| | symmetric_UGU-UGU; 58_3-3_bulge-symmetric_UGC-AAU; 69_3-3_bulge- |
| | symmetric_GUC-CCA |
| 874 | -6_6-6_internal_loop-symmetric_UUCAGA-AGGCUU; 5_1-1_mismatch_A- |
| | C; 20_6-6_internal_loop-symmetric_UGGAUC-UUGUGU |
| 17 | 3_1-1_mismatch_A-C; 22 6-6_internal_loop-symmetric_GAUCCU-CCCCGA |
| 1585 | -6_6-6_internal_loop-symmetric_UUCAGA-GGAACU; 3_1-1_mismatch_A- |
| | C; 33_6-6_internal_loop-symmetric_AGAUUU-CUGACC; 53_5- |
| | 5_internal_loop-symmetric_AUGAG-GAAAA; 72_5-5_internal_loop- |
| | symmetric_ACAAU-UCAAG |
| 757 | -7_6-6_internal_loop-symmetric_GUUCAG-AAGCAA; 5_1-1_mismatch_A- |
| | C; 22_6-6_internal_loop-symmetric_GAUCCU-UUCCCG |
| 1581 | -6_6-6_internal_loop-symmetric_UUCAGA-GGAACU; 3_1-1_mismatch_A- |
| | C; 33_6-6_internal_loop-symmetric_AGAUUU-CUGACC; 51_5- |
| | 5_internal_loop-symmetric_UGAUG-GUAGU; 68_5-5_internal_loop- |
| | symmetric_UGUCA-GCAGU |
| 1538 | -7_2-2_bulge-symmetric_AG-GA; 6_2-2_bulge-symmetric_AU-UC; 20_2- |
| | 2_bulge-symmetric_UG-AU; 34_2-2_bulge-symmetric_GA-CA; 48_2- |
| | 2_bulge-symmetric_GU-CG; 62_2-2_bulge-symmetric_GA-GA; 76_2- |
| | 2_bulge-symmetric_UA-CC |
| 8 | 3_1-1_mismatch_A-C; 20_6-6_internal_loop-symmetric_UGGAUC-ACAGGU |
| 1002 | -5_6-6_internal_loop-symmetric_UCAGAG-AAGGCU; 5_1-1_mismatch_A- |
| | C; 22_6-6_internal_loop-symmetric_GAUCCU-CCCUAA |
| 1566 | -6_6-6_internal_loop-symmetric_UUCAGA-GGAACU; 3_1-1_mismatch_A- |
| | C; 33_6-6_internal_loop-symmetric_AGAUUU-CUGACC; 45_2-2_bulge- |
| | symmetric_UU-CC; 53_2-2_bulge-symmetric_AU-CG; 61_2-2_bulge- |
| | symmetric_AG-GA; 69_2-2_bulge-symmetric_GU-UG; 77_2-2_bulge- |
| | symmetric AU-CC |
| 486 | -10_6-6_internal_loop-symmetric_UUAGUU-CCAGUC; 42_6- |
| | 6_internal_loop-symmetric_UCUUUU-UGUCCC |
| 1552 | -3_2-2_bulge-symmetric_AU-CA; 14_2-2_bulge-symmetric_UC-UU; 32_2- |
| | 2_bulge-symmetric_AA-GG; 50_2-2_bulge-symmetric_GU-CG; 68_2- |
| | 2_bulge-symmetric_UG-GC |
| 505 | -9_6-6_internal_loop-symmetric_UAGUUC-ACGUCC; 5_1-1_mismatch_A-C; |
| | 20_6-6_internal_loop-symmetric_UGGAUC-UCGGGU |
| 635 | -8_6-6_internal_loop-symmetric_AGUUCA-GAUUGA; 5_1-1_mismatch_A- |
| | C; 22_6-6_internal_loop-symmetric_GAUCCU-CCCUGG |
| 606 | -9_6-6_internal_loop-symmetric_UAGUUC-AUUUUU; 42_6-6_internal_loop- |
| | symmetric_UCUUUU-CCUCAC |
| 884 | -6_6-6_internal_loop-symmetric_UUCAGA-GGUUAU; 5_1-1_mismatch_A- |
| | C; 22_6-6_internal_loop-symmetric_GAUCCU-CCUCCA |
| 1054 | -5_6-6_internal_loop-symmetric_UCAGAG-GAGAUC; 33_6-6_internal_loop- |
| | symmetric_AGAUUU-CCUGGA |
| 880 | -6_6-6_internal_loop-symmetric_UUCAGA-ACCUUC; 21_6-6_internal_loop- |
| | symmetric_GGAUCC-CUCGAG |
| 1411 | -2_6-6_internal_loop-symmetric_GAGAUA-AGCGGG; 5_1-1_mismatch_A- |
| | C; 42_6-6_internal_loop-symmetric_UCUUUU-CCUCCC |
| 1568 | -6_6-6_internal_loop-symmetric_UUCAGA-GGAACU; 3_1-1_mismatch_A- |
| | C; 33_6-6_internal_loop-symmetric_AGAUUU-CUGACC; 45_4-4_bulge- |
| | symmetric_UUUG-GUAU; 55_4-4_bulge-symmetric_GAGU-CAAG; 65_4- |
| | 4_bulge-symmetric_AUAU-CAAA; 75_4-4_bulge-symmetric_AUAU-UUCG |
| 871 | -6_6-6_internal_loop-symmetric_UUCAGA-GUUCAC; 0_1-1_mismatch_A-C; |
| | 20_6-6_internal_loop-symmetric_UGGAUC-CUGUGU |
| 1580 | -6_6-6_internal_loop-symmetric_UUCAGA-GGAACU; 3_1-1_mismatch_A- |
| | C; 33_6-6_internal_loop-symmetric_AGAUUU-CUGACC; 51_4-4_bulge- |
| | symmetric_UGAU-CUAC; 67_4-4_bulge-symmetric_AUGU-UGUC |
| 1539 | -6_3-3_bulge-symmetric_AGA-CAG; 7_3-3_bulge-symmetric_UGC-AAC; |
| | 22_3-3_bulge-symmetric_GAU-UAG; 37_3-3_bulge-symmetric_UUG-GUU; |
| | 52_3-3_bulge-symmetric_GAU-CGG; 67_3-3_bulge-symmetric_AUG-ACC |
| 14 | 5_1-1_mismatch_A-C; 21_6-6_internal_loop-symmetric_GGAUCC-UCGCGG |
| 892 | -6_6-6_internal_loop-symmetric_UUCAGA-AAUCGC; 3_1-1_mismatch_A-C; |
| | 24_6-6_internal_loop-symmetric_UCCUAU-UGACCU |
| 1116 | -4_6-6_internal_loop-symmetric_CAGAGA-CGAAAC; 22_6-6_internal_loop- |
| | symmetric_GAUCCU-CCCCAG |
| 15 | 21_6-6_internal_loop-symmetric_GGAUCC-CCCCAA |
| 1586 | -6_6-6_internal_loop-symmetric_UUCAGA-GGAACU; 3_1-1_mismatch_A- |
| | C; 33_6-6_internal_loop-symmetric_AGAUUU-CUGACC; 55_2-2_bulge- |
| | symmetric_GA-AA; 73_2-2_bulge-symmetric_CA-CC |
| 593 | -9_6-6_internal_loop-symmetric_UAGUUC-CGUGAU; 0_1-1_mismatch_A- |
| | C; 40_6-6_internal_loop-symmetric_CAUCUU-CCCUCC |
| 10 | 5_1-1_mismatch_A-C; 20_6-6_internal_loop-symmetric_UGGAUC-UUAGAU |
| 977 | -6_6-6_internal_loop-symmetric_UUCAGA-AGCAUC; 3_1-1_mismatch_A-C; |
| | 42_6-6_internal_loop-symmetric_UCUUUU-CUGUCC |
| 1578 | -6_6-6_internal_loop-symmetric_UUCAGA-GGAACU; 3_1-1_mismatch_A- |
| | C; 33_6-6_internal_loop-symmetric_AGAUUU-CUGACC; 51_2-2_bulge- |
| | symmetric_UG-GC; 65_2-2_bulge-symmetric_AU-UG |
| 1579 | -6_6-6_internal_loop-symmetric_UUCAGA-GGAACU; 3_1-1_mismatch_A- |
| | C; 33_6-6_internal_loop-symmetric_AGAUUU-CUGACC; 51_3-3_bulge- |
| | symmetric_UGA-AGC; 66_3-3_bulge-symmetric_UAU-UAC |
| 747 | -7_6-6_internal_loop-symmetric_GUUCAG-AAAUUG; 3_1-1_mismatch_A- |
| | C; 20_6-6_internal_loop-symmetric_UGGAUC-AAAAGU |
| 1577 | -6_6-6_internal_loop-symmetric_UUCAGA-GGAACU; 3_1-1 mismatch_A- |
| | C; 33_6-6_internal_loop-symmetric_AGAUUU-CUGACC; 49_5- |
| | 5_internal_loop-symmetric_UGUGA-GCCUU; 64_5-5_internal_loop- |
| | symmetric_GAUAU-CUUAA |
| 748 | -7_6-6_internal_loop-symmetric_GUUCAG-ACGUCG; 5_1-1_mismatch_A-C; |
| | 20_6-6_internal_loop-symmetric_UGGAUC-CGCAAU |
| 873 | -6_6-6_internal_loop-symmetric_UUCAGA-CAUUAU; 4_1-1_mismatch_A- |
| | C; 20_6-6_internal_loop-symmetric_UGGAUC-AGCGUU |
| 494 | -10_6-6_internal_loop-symmetric_UUAGUU-CGUACC; 4_1-1_mismatch_A- |
| | C; 44_6-6_internal_loop-symmetric_UUUUGU-CCCUCC |

Targeting Position 4

| | |
|---|---|
| 1575 | -6_6-6_internal_loop-symmetric_UUCAGA-GGAACU; 3_1-1_mismatch_A- |
| | C; 33_6-6_internal_loop-symmetric_AGAUUU-CUGACC; 49_3-3_bulge- |
| | symmetric_UGU-CUU; 62_3-3_bulge-symmetric_GAG-GAG; 75_3-3_bulge- |
| | symmetric_AUA-GUG |
| 1573 | -6_6-6_internal_loop-symmetric_UUCAGA-GGAACU; 3_1-1_mismatch_A- |
| | C; 33_6-6_internal_loop-symmetric_AGAUUU-CUGACC; 47_5- |
| | 5_internal_loop-symmetric_UGUGU-UGAAU; 60_5-5_internal_loop- |
| | symmetric_CAGAG-GCACC; 73_5-5_internal_loop-symmetric_CAAUA- |
| | AAGUC |
| 1567 | -6_6-6_internal_loop-symmetric_UUCAGA-GGAACU; 3_1-1_mismatch_A- |
| | C; 33_6-6_internal_loop-symmetric_AGAUUU-CUGACC; 45_3-3_bulge- |
| | symmetric_UUU-UAU; 54_3-3_bulge-symmetric_UGA-CUU; 63_3-3_bulge- |
| | symmetric_AGA-GAC; 72_3-3_bulge-symmetric_ACA-GCA |
| 1569 | -6_6-6_internal_loop-symmetric_UUCAGA-GGAACU; 3_1-1_mismatch_A- |
| | C; 33_6-6_internal_loop-symmetric_AGAUUU-CUGACC; 45_5- |
| | 5_internal_loop-symmetric_UUUGU-UGUUC; 56_5-5_internal_loop- |
| | symmetric_AGUGC-CUUUC; 67_5-5_internal_loop-symmetric_AUGUC- |
| | CCAAA |
| 1570 | -6_6-6_internal_loop-symmetric_UUCAGA-GGAACU; 3_1-1_mismatch_A- |
| | C; 33_6-6_internal_loop-symmetric_AGAUUU-CUGACC; 47_2-2_bulge- |
| | symmetric_UG-AU; 57_2-2_bulge-symmetric_GU-CG; 67_2-2_bulge- |
| | symmetric_AU-UA; 77_2-2_bulge-symmetric_AU-UG |
| 1566 | -6_6-6_internal_loop-symmetric_UUCAGA-GGAACU; 3_1-1_mismatch_A- |
| | C; 33_6-6_internal_loop-symmetric_AGAUUU-CUGACC; 45_2-2_bulge- |
| | symmetric_UU-CC; 53_2-2_bulge-symmetric_AU-CG; 61_2-2_bulge- |
| | symmetric_AG-GA; 69_2-2_bulge-symmetric_GU-UG; 77_2-2_bulge- |
| | symmetric_AU-CC |
| 1572 | -6_6-6_internal_loop-symmetric_UUCAGA-GGAACU; 3_1-1_mismatch_A- |
| | C; 33_6-6_internal_loop-symmetric_AGAUUU-CUGACC; 47_4-4_bulge- |
| | symmetric_UGUG-ACCU; 59_4-4_bulge-symmetric_GCAG-GGUG; 71_4- |
| | 4_bulge-symmetric_CACA-CUUU |
| 1587 | -6_6-6_internal_loop-symmetric_UUCAGA-GGAACU; 3_1-1_mismatch_A- |
| | C; 33_6-6_internal_loop-symmetric_AGAUUU-CUGACC; 55_3-3_bulge- |
| | symmetric_GAG-GCG; 74_3-3 bulge-symmetric_AAU-UCA |
| 1571 | -6_6-6_internal_loop-symmetric_UUCAGA-GGAACU; 3_1-1_mismatch_A- |
| | C; 33_6-6_internal_loop-symmetric_AGAUUU-CUGACC; 47_3-3_bulge- |
| | symmetric_UGU-UGU; 58_3-3_bulge-symmetric_UGC-AAU; 69_3-3_bulge- |
| | symmetric_GUC-CCA |
| 1574 | -6_6-6_internal_loop-symmetric_UUCAGA-GGAACU; 3_1-1_mismatch_A- |
| | C; 33_6-6_internal_loop-symmetric_AGAUUU-CUGACC; 49_2-2_bulge- |
| | symmetric_UG-GC; 61_2-2_bulge-symmetric_AG-AA; 73_2-2_bulge- |
| | symmetric_CA-AC |
| 1584 | -6_6-6_internal_loop-symmetric_UUCAGA-GGAACU; 3_1-1_mismatch_A- |
| | C; 33_6-6_internal_loop-symmetric_AGAUUU-CUGACC; 53_4-4_bulge- |
| | symmetric_AUGA-AGCC; 71_4-4_bulge-symmetric_CACA-AUAA |
| 1588 | -6_6-6_internal_loop-symmetric_UUCAGA-GGAACU; 3_1-1_mismatch_A- |
| | C; 33_6-6_internal_loop-symmetric_AGAUUU-CUGACC; 55_4-4_bulge- |
| | symmetric_GAGU-CAAA; 75_4-4_bulge-symmetric_AUAU-CGCG |
| 1054 | -5_6-6_internal_loop-symmetric_UCAGAG-GAGAUC; 33_6-6_internal_loop- |
| | symmetric_AGAUUU-CCUGGA |
| 1586 | -6_6-6_internal_loop-symmetric_UUCAGA-GGAACU; 3_1-1_mismatch_A- |
| | C; 33_6-6_internal_loop-symmetric_AGAUUU-CUGACC; 55_2-2_bulge- |
| | symmetric_GA-AA; 73_2-2_bulge-symmetric_CA-CC |
| 1585 | -6_6-6_internal_loop-symmetric_UUCAGA-GGAACU; 3_1-1_mismatch_A- |
| | C; 33_6-6_internal_loop-symmetric_AGAUUU-CUGACC; 53_5- |
| | 5_internal_loop-symmetric_AUGAG-GAAAA; 72_5-5_internal_loop- |
| | symmetric_ACAAU-UCAAG |
| 1581 | -6_6-6_internal_loop-symmetric_UUCAGA-GGAACU; 3_1-1_mismatch_A- |
| | C; 33_6-6_internal_loop-symmetric_AGAUUU-CUGACC; 51_5- |
| | 5_internal_loop-symmetric_UGAUG-GUAGU; 68_5-5_internal_loop- |
| | symmetric_UGUCA-GCAGU |
| 1578 | -6_6-6_internal_loop-symmetric_UUCAGA-GGAACU; 3_1-1_mismatch_A- |
| | C; 33_6-6_internal_loop-symmetric_AGAUUU-CUGACC; 51_2-2_bulge- |
| | symmetric_UG-GC; 65_2-2_bulge-symmetric_AU-UG |
| 1580 | -6_6-6_internal_loop-symmetric_UUCAGA-GGAACU; 3_1-1_mismatch_A- |
| | C; 33_6-6_internal_loop-symmetric_AGAUUU-CUGACC; 51_4-4_bulge- |
| | symmetric_UGAU-CUAC; 67_4-4_bulge-symmetric_AUGU-UGUC |
| 934 | -6_6-6_internal_loop-symmetric_UUCAGA-CACCUC; 0_1-1_mismatch_A-C; |
| | 33_6-6_internal_loop-symmetric_AGAUUU-UCCCUA |
| 72 | 3_1-1_mismatch_A-C; 33_6-6_internal_loop-symmetric_AGAUUU- |
| | UUGGGA |
| 1582 | -6_6-6_internal_loop-symmetric_UUCAGA-GGAACU; 3_1-1_mismatch_A- |
| | C; 33_6-6_internal_loop-symmetric_AGAUUU-CUGACC; 53_2-2_bulge- |
| | symmetric_AU-UA; 69_2-2_bulge-symmetric_GU-UA |
| 1066 | -5_6-6_internal_loop-symmetric_UCAGAG-AAUUAC; 3_1-1_mismatch_A- |
| | C; 36_6-6_internal_loop-symmetric_UUUGCA-CUCCUC |
| 1183 | -4_6-6_internal_loop-symmetric_CAGAGA-AUGGCC; 3_1-1_mismatch_A-C; |
| | 36_6-6_internal_loop-symmetric_UUUGCA-GAAUCC |
| 1577 | -6_6-6_internal_loop-symmetric_UUCAGA-GGAACU; 3_1-1_mismatch_A- |
| | C; 33_6-6_internal_loop-symmetric_AGAUUU-CUGACC; 49_5- |
| | 5_internal_loop-symmetric_UGUGA-GCCUU; 64_5-5_internal_loop- |
| | symmetric_GAUAU-CUUAA |
| 967 | -6_6-6_internal_loop-symmetric_UUCAGA-AUAAGU; 0_1-1_mismatch_A- |
| | C; 40_6-6_internal_loop-symmetric_CAUCUU-UGCUCC |
| 1568 | -6_6-6_internal_loop-symmetric_UUCAGA-GGAACU; 3_1-1_mismatch_A- |
| | C; 33_6-6_internal_loop-symmetric_AGAUUU-CUGACC; 45_4-4_bulge- |
| | symmetric_UUUG-GUAU; 55_4-4_bulge-symmetric_GAGU-CAAG; 65_4- |
| | 4_bulge-symmetric_AUAU-CAAA; 75_4-4_bulge-symmetric_AUAU-UUCG |
| 930 | -6_6-6_internal_loop-symmetric_UUCAGA-CAACAC; 3_1-1 mismatch_A-C; |
| | 32 6-6_internal_loop-symmetric_AAGAUU-UUGGCC |
| 566 | -9_6-6_internal_loop-symmetric_UAGUUC-UCCACC; 3_1-1_mismatch_A-C; |
| | 34 6-6_internal_loop-symmetric_GAUUUG-AUUGGG |
| 1463 | -1_6-6_internal_loop-symmetric_AGAUAU-UCCCUG; 4_1-1_mismatch_A-C; |
| | 32_6-6 internal_loop-symmetric_AAGAUU-CAGGGG |
| 1294 | -3_6-6_internal_loop-symmetric_AGAGAU-UCAAAC; 4_1-1_mismatch_A- |
| | C; 36_6-6_internal_loop-symmetric_UUUGCA-CCCCUC |
| 1293 | -3_6-6_internal_loop-symmetric_AGAGAU-CCAGGG; 3_1-1_mismatch_A- |
| | C; 36_6-6_internal_loop-symmetric_UUUGCA-GGGUCC |
| 1391 | -2_6-6_internal_loop-symmetric_GAGAUA-GCGGAG; 3_1-1_mismatch_A- |
| | C; 37_6-6_internal_loop-symmetric_UUGCAU-CACCCU |
| 1579 | -6_6-6_internal_loop-symmetric_UUCAGA-GGAACU; 3_1-1_mismatch_A- |
| | C; 33_6-6_internal_loop-symmetric_AGAUUU-CUGACC; 51_3-3_bulge- |
| | symmetric_UGA-AGC; 66_3-3_bulge-symmetric_UAU-UAC |
| 1583 | -6_6-6_internal_loop-symmetric_UUCAGA-GGAACU; 3_1-1_mismatch_A- |
| | C; 33_6-6_internal_loop-symmetric_AGAUUU-CUGACC; 53_3-3_bulge- |
| | symmetric_AUG-GCG; 70_3-3_bulge-symmetric_UCA-AUU |
| 944 | -6_6-6_internal_loop-symmetric_UUCAGA-GAAUUC; 3_1-1_mismatch_A- |
| | C; 35_6-6_internal_loop-symmetric_AUUUGC-UAUGCC |
| 815 | -7_6-6_internal_loop-symmetric_GUUCAG-GCCCCG; 3_1-1_mismatch_A-C; |
| | 34_6-6 internal_loop-symmetric_GAUUUG-GCCUGG |
| 1168 | -4_6-6_internal_loop-symmetric_CAGAGA-GUAGUC; 3_1-1_mismatch_A- |
| | C; 33_6-6_internal_loop-symmetric_AGAUUU-CGCGGA |
| 593 | -9_6-6_internal_loop-symmetric_UAGUUC-CGUGAU; 0_1-1_mismatch_A- |
| | C; 40_6-6_internal_loop-symmetric_CAUCUU-CCCUCC |
| 594 | -9_6-6_internal_loop-symmetric_UAGUUC-UCUUGC; 3_1-1_mismatch_A-C; |
| | 40_6-6_internal_loop-symmetric_CAUCUU-CCUUCC |
| 694 | -8_6-6_internal_loop-symmetric_AGUUCA-ACUCGA; 3_1-1_mismatch_A-C; |
| | 35_6-6_internal_loop-symmetric_AUUUGC-CUUCCC |
| 1576 | -6_6-6_internal_loop-symmetric_UUCAGA-GGAACU; 3_1-1_mismatch_A- |
| | C; 33_6-6_internal_loop-symmetric_AGAUUU-CUGACC; 49_4-4_bulge- |
| | symmetric_UGUG-GUAU; 63_4-4_bulge-symmetric_AGAU-UGGC |
| 1193 | -4_6-6_internal_loop-symmetric_CAGAGA-CGGGCA; 4_1-1_mismatch_A-C; |
| | 38_6-6_internal_loop-symmetric_UGCAUC-CCCCCU |
| 1051 | -5_6-6_internal_loop-symmetric_UCAGAG-AGAUAC; 3_1-1_mismatch_A- |
| | C; 33_6-6_internal_loop-symmetric_AGAUUU-CGCCCC |
| 1212 | -4_6-6_internal_loop-symmetric_CAGAGA-GACAUU; 3_1-1_mismatch_A- |
| | C; 42_6-6_internal_loop-symmetric_UCUUUU-UCGUCC |
| 806 | -7_6-6_internal_loop-symmetric_GUUCAG-GGCUAA; 4_1-1_mismatch_A- |
| | C; 32_6-6_internal_loop-symmetric_AAGAUU-CCUGCC |
| 1059 | -5_6-6_internal_loop-symmetric_UCAGAG-AUACCC; 34_6-6_internal_loop- |
| | symmetric_GAUUUG-GUUGGG |
| 1374 | -2_6-6_internal_loop-symmetric_GAGAUA-CGAGGG; 32_6-6_internal_loop- |
| | symmetric_AAGAUU-CGACCC |
| 195 | -12_6-6_internal_loop-symmetric_GAUUAG-GCCCGG; 5_1-1_mismatch_A- |
| | C; 33_6-6_internal_loop-symmetric_AGAUUU-UCGGGA |
| 358 | -11_6-6_internal_loop-symmetric_AUUAGU-CCCUCG; 4_1-1_mismatch_A- |
| | C; 42_6-6_internal_loop-symmetric_UCUUUU-UCCCUC |
| 1296 | -3_6-6_internal_loop-symmetric_AGAGAU-UGGAUG; 36_6-6_internal_loop- |
| | symmetric_UUUGCA-AAAACC |

Targeting Position 5

| | |
|---|---|
| 1575 | -6_6-6_internal_loop-symmetric_UUCAGA-GGAACU; 3_1-1_mismatch_A- |
| | C; 33_6-6_internal_loop-symmetric_AGAUUU-CUGACC; 49_3-3_bulge- |
| | symmetric_UGU-CUU; 62_3-3_bulge-symmetric_GAG-GAG; 75_3-3_bulge- |
| | symmetric_AUA-GUG |
| 1573 | -6_6-6_internal_loop-symmetric_UUCAGA-GGAACU; 3_1-1_mismatch_A- |
| | C; 33_6-6_internal_loop-symmetric_AGAUUU-CUGACC; 47_5- |
| | 5_internal_loop-symmetric_UGUGU-UGAAU; 60_5-5_internal_loop- |
| | symmetric_CAGAG-GCACC; 73_5-5_internal_loop-symmetric_CAAUA- |
| | AAGUC |
| 1569 | -6_6-6_internal_loop-symmetric_UUCAGA-GGAACU; 3_1-1_mismatch_A- |
| | C; 33_6-6_internal_loop-symmetric_AGAUUU-CUGACC; 45_5- |
| | 5_internal_loop-symmetric_UUUGU-UGUUC; 56_5-5_internal_loop- |
| | symmetric_AGUGC-CUUUC; 67_5-5_internal_loop-symmetric_AUGUC- |
| | CCAAA |
| 1574 | -6_6-6_internal_loop-symmetric_UUCAGA-GGAACU; 3_1-1 mismatch_A- |
| | C; 33_6-6_internal_loop-symmetric_AGAUUU-CUGACC; 49_2-2_bulge- |
| | symmetric_UG-GC; 61_2-2_bulge-symmetric_AG-AA; 73_2-2_bulge- |
| | symmetric_CA-AC |
| 1570 | -6_6-6_internal_loop-symmetric_UUCAGA-GGAACU; 3_1-1 mismatch_A- |
| | C; 33_6-6_internal_loop-symmetric_AGAUUU-CUGACC; 47_2-2_bulge- |
| | symmetric_UG-AU; 57_2-2_bulge-symmetric_GU-CG; 67_2-2_bulge- |
| | symmetric_AU-UA; 77_2-2_bulge-symmetric_AU-UG |
| 1572 | -6_6-6_internal_loop-symmetric_UUCAGA-GGAACU; 3_1-1_mismatch_A- |
| | C; 33_6-6_internal_loop-symmetric_AGAUUU-CUGACC; 47_4-4_bulge- |
| | symmetric_UGUG-ACCU; 59_4-4_bulge-symmetric_GCAG-GGUG; 71_4- |
| | 4_bulge-symmetric_CACA-CUUU |
| 1567 | -6_6-6_internal_loop-symmetric_UUCAGA-GGAACU; 3_1-1_mismatch_A- |
| | C; 33_6-6_internal_loop-symmetric_AGAUUU-CUGACC; 45_3-3_bulge- |
| | symmetric_UUU-UAU; 54_3-3_bulge-symmetric_UGA-CUU; 63_3-3_bulge- |
| | symmetric_AGA-GAC; 72_3-3_bulge-symmetric_ACA-GCA |
| 1587 | -6_6-6_internal_loop-symmetric_UUCAGA-GGAACU; 3_1-1_mismatch_A- |
| | C; 33_6-6_internal_loop-symmetric_AGAUUU-CUGACC; 55_3-3_bulge- |
| | symmetric_GAG-GCG; 74_3-3_bulge-symmetric_AAU-UCA |
| 1566 | -6_6-6_internal_loop-symmetric_UUCAGA-GGAACU; 3_1-1_mismatch_A- |
| | C; 33_6-6_internal_loop-symmetric_AGAUUU-CUGACC; 45_2-2_bulge- |
| | symmetric_UU-CC; 53_2-2_bulge-symmetric_AU-CG; 61_2-2_bulge- |
| | symmetric_AG-GA; 69_2-2_bulge-symmetric_GU-UG; 77_2-2_bulge- |
| | symmetric_AU-CC |
| 1571 | -6_6-6_internal_loop-symmetric_UUCAGA-GGAACU; 3_1-1_mismatch_A- |
| | C; 33_6-6_internal_loop-symmetric_AGAUUU-CUGACC; 47_3-3_bulge- |
| | symmetric_UGU-UGU; 58_3-3_bulge-symmetric_UGC-AAU; 69_3-3_bulge- |
| | symmetric_GUC-CCA |
| 1588 | -6_6-6_internal_loop-symmetric_UUCAGA-GGAACU; 3_1-1_mismatch_A- |
| | C; 33_6-6_internal_loop-symmetric_AGAUUU-CUGACC; 55_4-4_bulge- |
| | symmetric_GAGU-CAAA; 75_4-4_bulge-symmetric_AUAU-CGCG |
| 72 | 3_1-1_mismatch_A-C; 33_6-6_internal_loop-symmetric_AGAUUU- |
| | UUGGGA |
| 1586 | -6_6-6_internal_loop-symmetric_UUCAGA-GGAACU; 3_1-1_mismatch_A- |
| | C; 33_6-6_internal_loop-symmetric_AGAUUU-CUGACC; 55_2-2_bulge- |
| | symmetric_GA-AA; 73_2-2_bulge-symmetric_CA-CC |
| 1584 | -6_6-6_internal_loop-symmetric_UUCAGA-GGAACU; 3_1-1_mismatch_A- |
| | C; 33_6-6_internal_loop-symmetric_AGAUUU-CUGACC; 53_4-4_bulge- |
| | symmetric_AUGA-AGCC; 71_4-4_bulge-symmetric_CACA-AUAA |
| 1581 | -6_6-6_internal_loop-symmetric_UUCAGA-GGAACU; 3_1-1_mismatch_A- |
| | C; 33_6-6_internal_loop-symmetric_AGAUUU-CUGACC; 51_5- |
| | 5_internal_loop-symmetric_UGAUG-GUAGU; 68_5-5_internal_loop- |
| | symmetric_UGUCA-GCAGU |
| 1578 | -6_6-6_internal_loop-symmetric_UUCAGA-GGAACU; 3_1-1 mismatch_A- |
| | C; 33_6-6_internal_loop-symmetric_AGAUUU-CUGACC; 51_2-2_bulge- |
| | symmetric_UG-GC; 65_2-2_bulge-symmetric_AU-UG |
| 1585 | -6_6-6_internal_loop-symmetric_UUCAGA-GGAACU; 3_1-1_mismatch_A- |
| | C; 33_6-6_internal_loop-symmetric_AGAUUU-CUGACC; 53_5- |
| | 5_internal_loop-symmetric_AUGAG-GAAAA; 72_5-5_internal_loop- |
| | symmetric_ACAAU-UCAAG |
| 1582 | -6_6-6_internal_loop-symmetric_UUCAGA-GGAACU; 3_1-1_mismatch_A- |
| | C; 33_6-6_internal_loop-symmetric_AGAUUU-CUGACC; 53_2-2_bulge- |
| | symmetric_AU-UA; 69_2-2_bulge-symmetric_GU-UA |
| 1580 | -6_6-6_internal_loop-symmetric_UUCAGA-GGAACU; 3_1-1_mismatch_A- |
| | C; 33_6-6_internal_loop-symmetric_AGAUUU-CUGACC; 51_4-4_bulge- |
| | symmetric_UGAU-CUAC; 67_4-4_bulge-symmetric_AUGU-UGUC |
| 1183 | -4_6-6_internal_loop-symmetric_CAGAGA-AUGGCC; 3_1-1_mismatch_A-C; |
| | 36 6-6_internal_loop-symmetric_UUUGCA-GAAUCC |
| 1568 | -6_6-6_internal_loop-symmetric_UUCAGA-GGAACU; 3_1-1_mismatch_A- |
| | C; 33_6-6_internal_loop-symmetric_AGAUUU-CUGACC; 45_4-4_bulge- |
| | symmetric_UUUG-GUAU; 55_4-4_bulge-symmetric_GAGU-CAAG; 65_4- |
| | 4_bulge-symmetric_AUAU-CAAA; 75_4-4_bulge-symmetric_AUAU-UUCG |
| 1066 | -5_6-6_internal_loop-symmetric_UCAGAG-AAUUAC; 3_1-1_mismatch_A- |
| | C; 36_6-6_internal_loop-symmetric_UUUGCA-CUCCUC |
| 1391 | -2_6-6_internal_loop-symmetric_GAGAUA-GCGGAG; 3_1-1_mismatch_A- |
| | C; 37_6-6_internal_loop-symmetric_UUGCAU-CACCCU |
| 1168 | -4_6-6_internal_loop-symmetric_CAGAGA-GUAGUC; 3_1-1_mismatch_A- |
| | C; 33_6-6_internal_loop-symmetric_AGAUUU-CGCGGA |
| 1293 | -3_6-6_internal_loop-symmetric_AGAGAU-CCAGGG; 3_1-1_mismatch_A- |
| | C; 36_6-6_internal_loop-symmetric_UUUGCA-GGGUCC |
| 1577 | -6_6-6_internal_loop-symmetric_UUCAGA-GGAACU; 3_1-1_mismatch_A- |
| | C; 33_6-6_internal_loop-symmetric_AGAUUU-CUGACC; 49_5- |
| | 5_internal_loop-symmetric_UGUGA-GCCUU; 64_5-5_internal_loop- |
| | symmetric_GAUAU-CUUAA |
| 1054 | -5_6-6_internal_loop-symmetric_UCAGAG-GAGAUC; 33_6-6_internal_loop- |
| | symmetric_AGAUUU-CCUGGA |
| 566 | -9_6-6_internal_loop-symmetric_UAGUUC-UCCACC; 3_1-1_mismatch_A-C; |
| | 34_6-6_internal_loop-symmetric_GAUUUG-AUUGGG |
| 1579 | -6_6-6_internal_loop-symmetric_UUCAGA-GGAACU; 3_1-1_mismatch_A- |
| | C; 33_6-6_internal_loop-symmetric_AGAUUU-CUGACC; 51_3-3_bulge- |
| | symmetric_UGA-AGC; 66_3-3_bulge-symmetric_UAU-UAC |
| 930 | -6_6-6_internal_loop-symmetric_UUCAGA-CAACAC; 3_1-1_mismatch_A-C; |
| | 32_6-6_internal_loop-symmetric_AAGAUU-UUGGCC |
| 694 | -8_6-6_internal_loop-symmetric_AGUUCA-ACUCGA; 3_1-1_mismatch_A-C; |
| | 35_6-6 internal_loop-symmetric_AUUUGC-CUUCCC |
| 944 | -6_6-6_internal_loop-symmetric_UUCAGA-GAAUUC; 3_1-1_mismatch_A- |
| | C; 35_6-6_internal_loop-symmetric_AUUUGC-UAUGCC |
| 195 | -12_6-6_internal_loop-symmetric_GAUUAG-GCCCGG; 5_1-1 mismatch_A- |
| | C; 33_6-6_internal_loop-symmetric_AGAUUU-UCGGGA |
| 1583 | -6_6-6_internal_loop-symmetric_UUCAGA-GGAACU; 3_1-1_mismatch_A- |
| | C; 33_6-6_internal_loop-symmetric_AGAUUU-CUGACC; 53_3-3_bulge- |
| | symmetric_AUG-GCG; 70_3-3_bulge-symmetric_UCA-AUU |
| 815 | -7_6-6_internal_loop-symmetric_GUUCAG-GCCCCG; 3_1-1_mismatch_A-C; |
| | 34_6-6_internal_loop-symmetric_GAUUUG-GCCUGG |
| 1576 | -6_6-6_internal_loop-symmetric_UUCAGA-GGAACU; 3_1-1_mismatch_A- |
| | C; 33_6-6_internal_loop-symmetric_AGAUUU-CUGACC; 49_4-4_bulge- |
| | symmetric_UGUG-GUAU; 63_4-4_bulge-symmetric_AGAU-UGGC |
| 1051 | -5_6-6_internal_loop-symmetric_UCAGAG-AGAUAC; 3_1-1_mismatch_A- |
| | C; 33_6-6_internal_loop-symmetric_AGAUUU-CGCCCC |
| 1411 | -2_6-6_internal_loop-symmetric_GAGAUA-AGCGGG; 5_1-1_mismatch_A- |
| | C; 42_6-6_internal_loop-symmetric_UCUUUU-CCUCCC |
| 24 | 5_1-1 mismatch_A-C; 23_6-6_internal_loop-symmetric_AUCCUA-CGUCCG |
| 1163 | -4_6-6_internal_loop-symmetric_CAGAGA-AUGAGA; 3_1-1_mismatch_A- |
| | C; 32_6-6_internal_loop-symmetric_AAGAUU-CCCAGC |
| 935 | -6_6-6_internal_loop-symmetric_UUCAGA-GGAACU; 3_1-1_mismatch_A- |
| | C; 33_6-6_internal_loop-symmetric_AGAUUU-CUGACC |
| 680 | -8_6-6_internal_loop-symmetric_AGUUCA-ACCCGA; 3_1-1 mismatch_A-C; |
| | 32_6-6_internal_loop-symmetric_AAGAUU-CUGGAC |
| 1212 | -4_6-6_internal_loop-symmetric_CAGAGA-GACAUU; 3_1-1_mismatch_A- |
| | C; 42_6-6_internal_loop-symmetric_UCUUUU-UCGUCC |
| 594 | -9_6-6_internal_loop-symmetric_UAGUUC-UCUUGC; 3_1-1_mismatch_A-C; |
| | 40_6-6_internal_loop-symmetric_CAUCUU-CCUUCC |
| 1185 | -4_6-6_internal_loop-symmetric_CAGAGA-AAGGUC; 5_1-1_mismatch_A- |
| | C; 36_6-6_internal_loop-symmetric_UUUGCA-GACCUC |
| 1463 | -1_6-6_internal_loop-symmetric_AGAUAU-UCCCUG; 4_1-1_mismatch_A-C; |
| | 32_6-6_internal_loop-symmetric_AAGAUU-CAGGGG |
| 1058 | -5_6-6_internal_loop-symmetric_UCAGAG-ACGCAC; 5_1-1_mismatch_A-C; |
| | 34_6-6_internal_loop-symmetric_GAUUUG-AUCGGG |
| 810 | -7_6-6_internal_loop-symmetric_GUUCAG-AGUUUA; 3_1-1_mismatch_A- |
| | C; 33_6-6_internal_loop-symmetric_AGAUUU-CCUAUA |
| 392 | -10_6-6_internal_loop-symmetric_UUAGUU-UGUCUU; 5_1-1_mismatch_A- |
| | C; 23_6-6_internal_loop-symmetric_AUCCUA-AUUAUG |
| 1104 | -5_6-6_internal_loop-symmetric_UCAGAG-GGGUCC; 3_1-1_mismatch_A-C; |
| | 44_6-6_internal_loop-symmetric_UUUUGU-UGCCCC |

### Example 11

Targeting of the DUX4 polyA Site in Cells

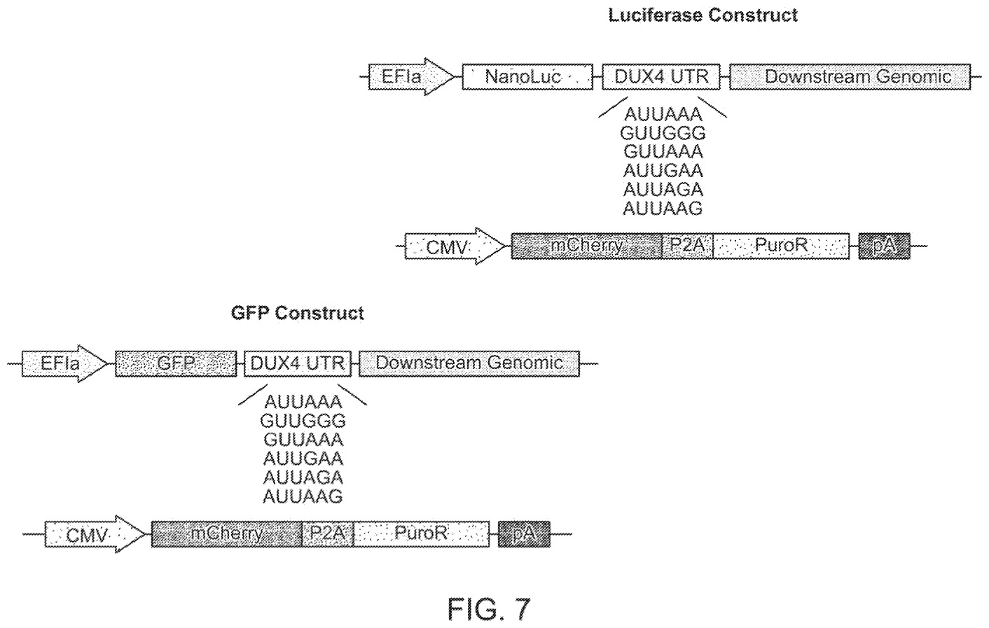

This example describes the change in expression of reporters fused to mutated DUX4-FL polyA site adenosines. To test the expression of the DUX4-FL polyA site in cells, two DUX4-FL fluorescent reporters were designed and generated. A GFP reporter construct (EF1a-GFP-DUX4flwt3′UTR) and a luciferase reporter construct (EF1a-luciferase-DUX4flwt3′UTR), were tested in immortalized myoblasts (LHCN-M2 cells, also known as LHCNs). A schematic of the luciferase and GFP constructs are shown in FIG. 7. Both reporters were engineered to include alternative versions where specific adenosine(s) at the polyA site were mutated to G to test their role in mRNA and protein levels. In addition to the unaltered version (or wild type version) ATTAAA, the alternate versions included ATTAAG; ATTAGA; ATTGAA; GTTAAA; and GTTGGG. To determine the RNA sequence of these polyA sites, all T bases are substituted with U bases. Given that if a said mutation(s) resulted in lower mRNA/protein levels, there would be less GFP/luciferase mRNA and protein in cells expressing mutant constructs.

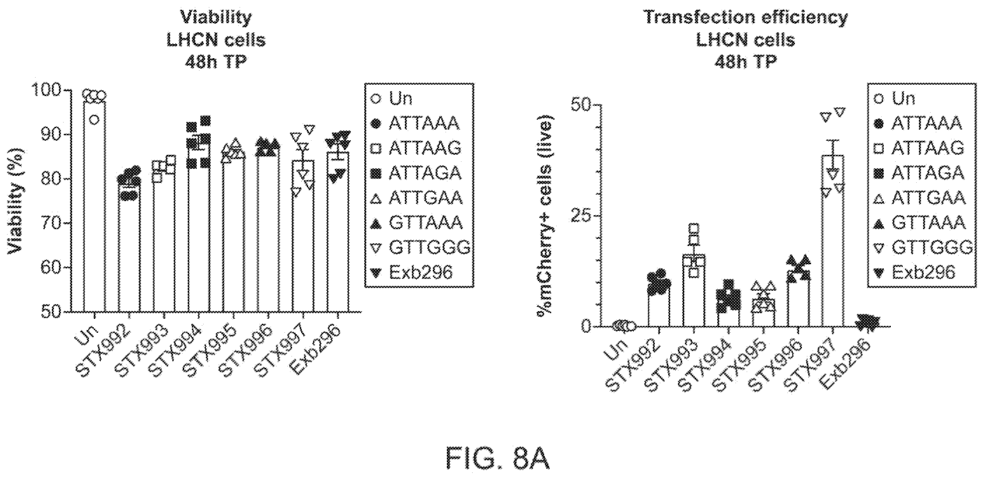

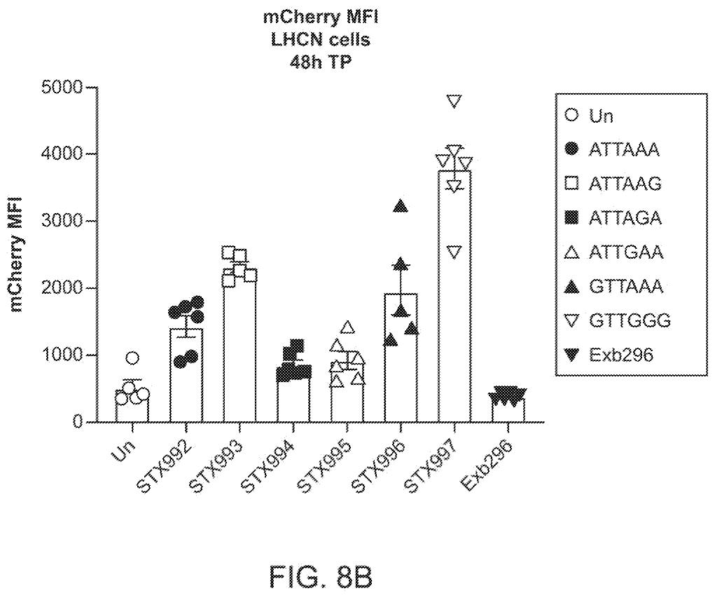

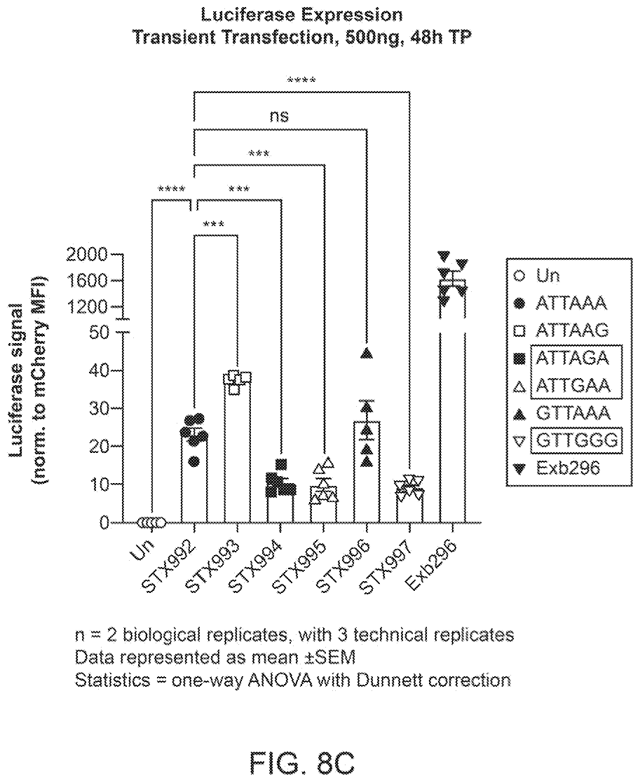

To determine if mutations of the DUX4-FL polyA site in LHCN-M2 cells changed expression of the reporter, the cells were transfected with the luciferase construct. Immortalized LHCN muscle cells were forward plated at 10K cells/well and transfected the next day with 500 ng of DNA plasmid (Lipofectamine 2000, 1:3 DNA:reagent ratio). The cells were processed for viability and analyzed via a mCherry flow analysis. The supernatants were examined by a luciferase assay 48 hours post-transfections. Results from the luciferase experiment are shown in FIGS. 8A-8C. The cells were ˜80%-90% viable after the transfection and had transfection efficiencies of ˜7-40% (mCherry positive), as shown in FIG. 8A. The different transfection efficiencies may be due to the difficulties associated with transfecting muscle cells. The luciferase expression was normalized to mCherry median fluorescent intensity (MFI) (shown in FIG. 8B). As shown in FIG. 8C, after normalization, the constructs STX994 (ATTAGA), STX995 (ATTGAA), and STX997 (GTTGGG) resulted in significant downregulation of the WT DUX4-3′UTR luciferase signal STX992 (ATTAAA). STX993 (ATTAAG) resulted in a significant increase in luciferase signal, while STX996 (GTTAAA) showed no change. The “Un” was the untransfected control and “Exb296” was the positive control. These results indicate, DUX4 can be downregulated in a muscle cell by mutating the DUX4-FL polyA site.

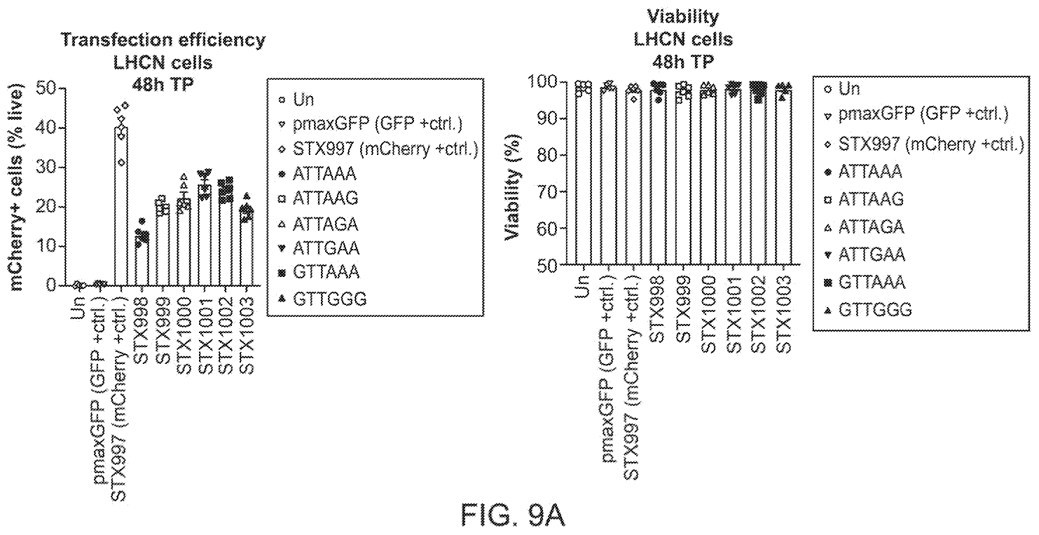

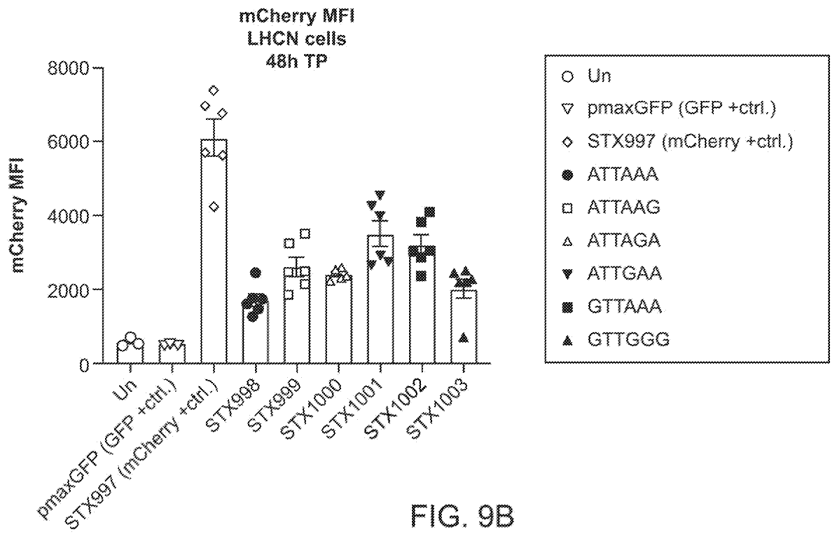

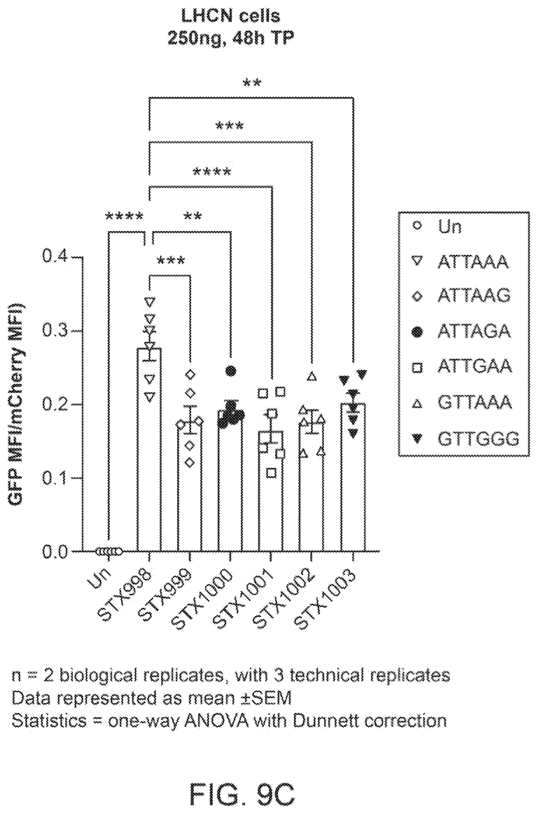

To determine if mutations of the DUX4-FL polyA site in LHCN-M2 cells changed expression of the reporter, the cells were transfected with the GFP construct. Immortalized LHCN muscle cells were forward plated at 10K cells/well and transfected the next day with 250 ng of DNA plasmid (Lipofectamine 2000, 1:3 DNA:reagent ratio). The cells were processed for viability and analyzed via mCherry/GFP flow analysis 48 hours post-transfections. The results from the GFP experiment are shown in FIGS. 9A-9C. The cells were greater than 90% viable after the transfection and had transfection efficiencies of ˜10-40% (mCherry positive), as shown in FIG. 9A. The different transfection efficiencies may be due to the difficulties associated with transfecting muscle cells. The GFP MFI was normalized to mCherry MFI (shown in FIG. 9B). As shown in FIG. 9C, after normalization, all constructs, STX999 (ATTAAG), STX1000 (ATTAGA), STX1001 (ATTGAA), STX1002 (GTTAAA) and STX1003 (GTTGGG) resulted in significant downregulation of the WT DUX4-3′UTR GFP signal STX998 (ATTAAA). The “Un” was the untransfected control. These results indicate, DUX4 can be downregulated in a cell by mutating the DUX4-FL polyA site. These constructs (luciferase and GFP) can also be used with guide RNAs described herein to test expression changes resulting from RNA editing of the DUX4 polyA site.

### Example 12

Targeting of the DUX4 polyA Signal Sequence in Cells

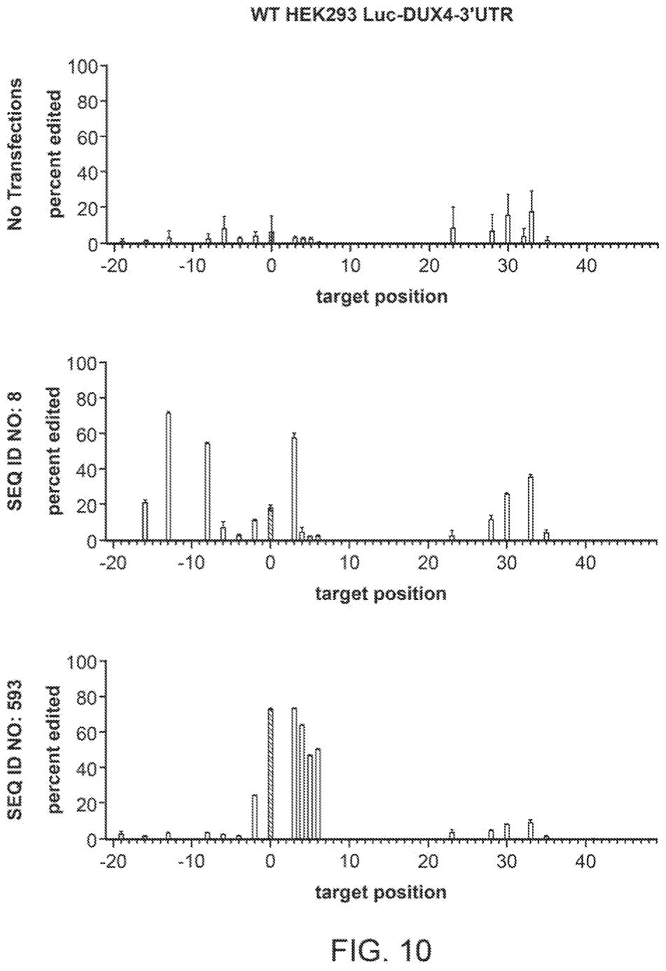

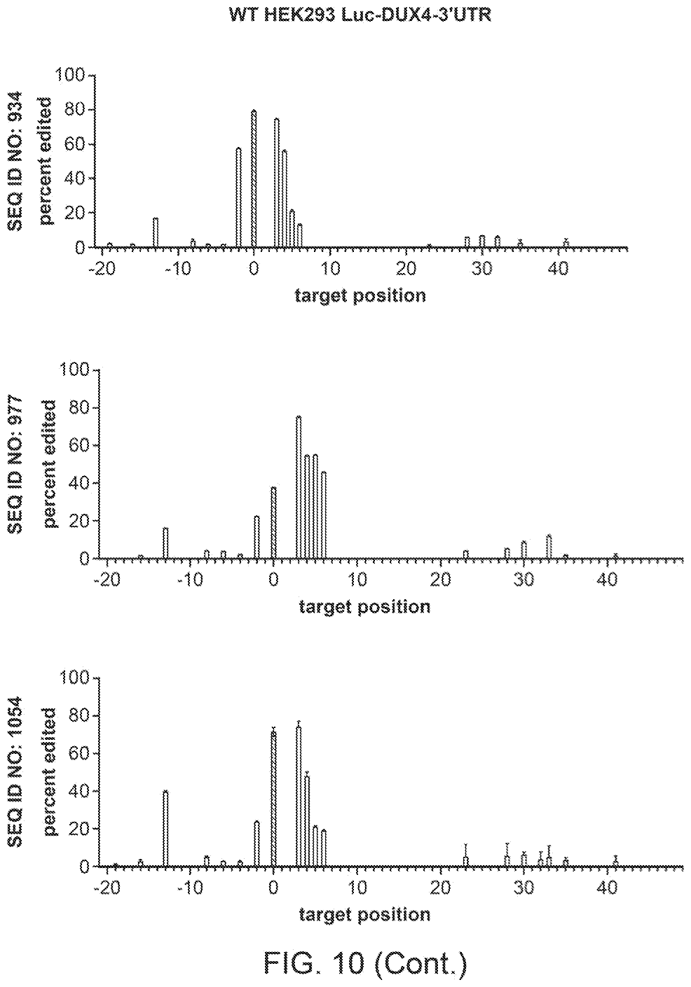

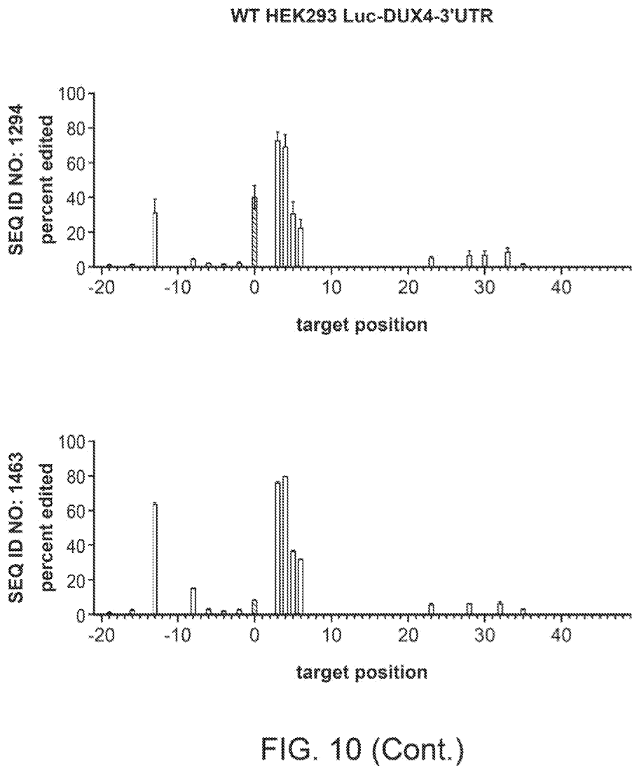

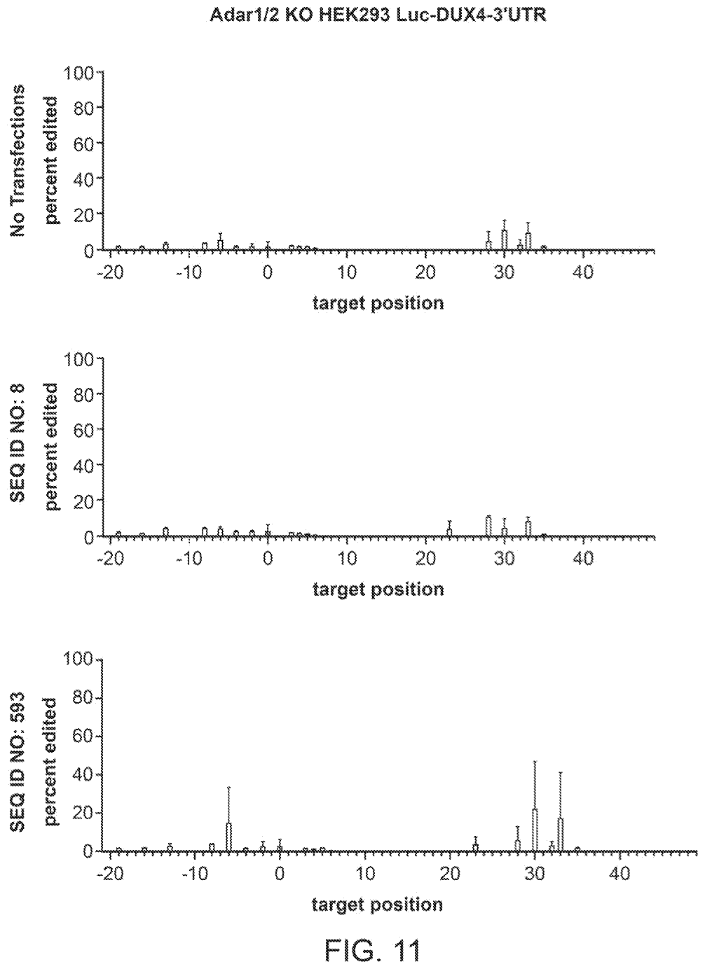

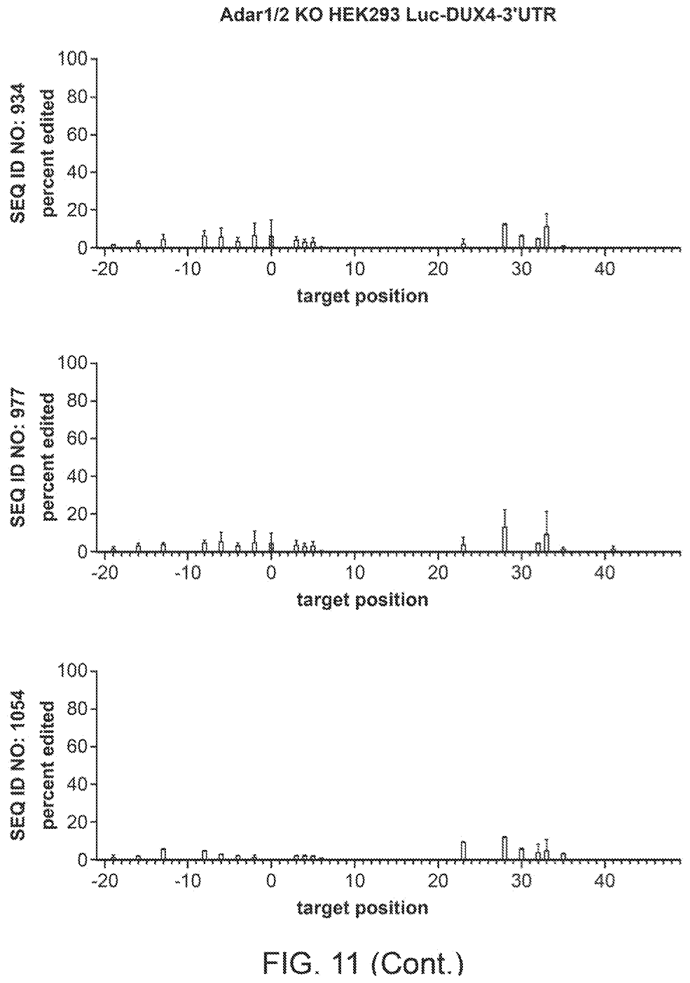

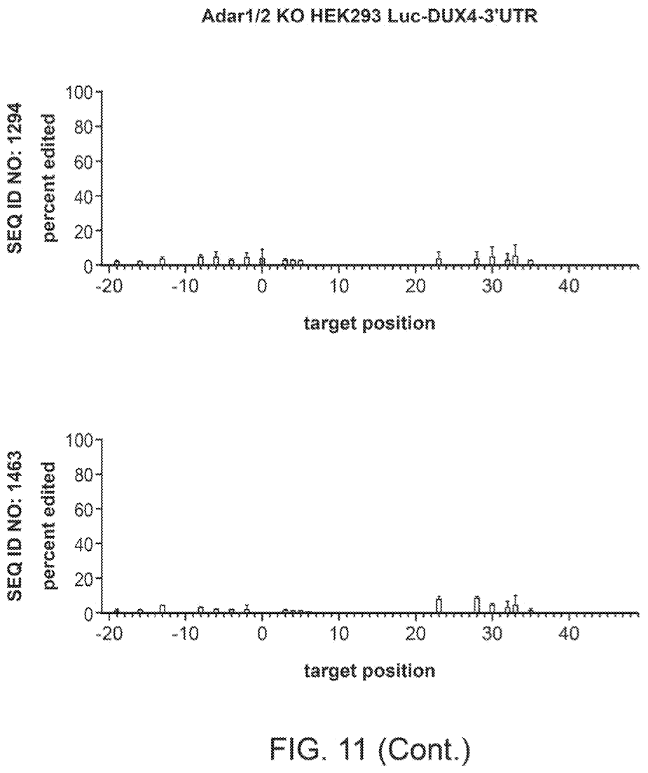

HEK cells were transfected with a DUX4-luciferase reporter that was stably integrated via the Piggybac system. The same DUX4-luciferase reporter was used for the ADAR 1/2 Knockout (KO) cells. To test editing of the DUX4 polyA site, seven gRNAs were tested and a no transfection control was tested. The seven gRNAs that were tested were SEQ ID NO: 8, SEQ ID NO: 593, SEQ ID NO: 934, SEQ ID NO: 977, SEQ ID NO: 1054, SEQ ID NO: 1294, and SEQ ID NO: 1463. Cells were transfected with a plasmid individually encoding each one of the seven gRNAs. The cells were collected 48 hours post transfection, and RNA was collected, converted to DNA by reverse transcriptase and sequenced via Sanger sequencing. FIG. 11 shows limited to no editing in the ADAR ½ knockout cells with the 7 guides tested. FIG. 10 shows the editing in HEK cells comprising a functional ADAR 1. For example, the SEQ ID NO: 8 guide facilitated high levels of editing (about 60%) at position 3 of the DUX 4 poly A tail, which is the third A from the 5′ end of ATTAAA. Editing with the SEQ ID NO: 593 guide had high levels of editing (about 70%) at positions 0 (the first A of ATTAAA), and position 3 of the DUX 4 poly A tail. Greater than 40% editing was also seen at positions 4, and 5 of the poly A tail with SEQ ID NO: 593, which is the third A and the fourth A from the 5′ end of ATTAAA, respectively. Editing with the SEQ ID NO: 934 guide had high levels of editing (about 78%) at position 0, about 75% editing at position 3, and about 60% editing at position 4 of the DUX 4 poly A tail. Editing with the SEQ ID NO: 977 guide had high levels of editing (about 75%) at position 3, and about 60% editing at positions 4 and 5. Editing with the SEQ ID NO: 1054 guide had high levels of editing (greater than about 70%) at positions 0 and 3, and about 40% editing at position 4. Editing with the SEQ ID NO: 1294 guide had high levels of editing (about 70% to 75%) at positions 3 and 4, and about 40% editing at position 0. Editing with the SEQ ID NO: 1463 guide had high levels of editing (about 80%) at positions 3 and 4. These results indicate that DUX-4 mRNA can be edited at a high efficiency in cells.

mRNA Knockdown. RNA preps (2 biological replicates) from cells used to quantify the above editing levels were also analyzed for mRNA knockdown by qPCR for mRNA knockdown. qPCR data was normalized to GAPDH mRNA and the average fold change of two biological replicates is presented in TABLE 4 below, with the no transfection control being set to 1. Knockdown was observed for all the engineered guide RNAs tested in the WT cell background, while ADAR ½ KO cells showed mostly no knock down.

TABLE 4
DUX4 mRNA Knockdown (Fold Change Normalized to GAPDH n = 2)

| | No | SEQ ID | SEQ ID | SEQ ID | SEQ ID | SEQ ID | SEQ ID | SEQ ID |
|---|---|---|---|---|---|---|---|---|
| | Transfection | NO: 8 | NO: 593 | NO: 934 | NO: 977 | NO: 1054 | NO: 1294 | NO: 1463 |
| WT | 1.00 | 0.367 | 0.50085 | 0.42213 | 0.39594 | 0.2907 | 0.51247 | 0.70405 |
| ADAR | 1.00 | 0.82464 | 0.947 | 1.048593 | 0.8753 | 0.817242 | 0.734 | 0.853 |
| ½ KO | | | | | | | | |

### Example 13

Reduction of DUX4 mRNA Transcript

This example describes the reduction of DUX4 mRNA levels in cells. Human FSHD-derived myoblasts are transfected with any of the engineered guide RNAs described herein (e.g., any one of SEQ ID NO: 2-SEQ ID NO: 1589). The cells are samples at 0, 12, 24, and 48 hours after transfection. After sampling the cells, the cells are lysed and RNA is purified. The RNA is converted to DNA with a reverse transcriptase and RNA levels are determined by quantitative real time polymerase chain reaction (qRT-PCR). relative and absolute expression levels are determined for DUX4 mRNA levels. DUX4 mRNA levels decrease after transfection with the engineered guide RNA.

### Example 14

### Reduction of DUX4 Downstream Protein Level

This example describes the reduction of a protein downstream of DUX4. Human FSHD-derived myoblasts are transfected with any of the engineered guide RNAs described herein (e.g., any one of SEQ ID NO: 2-SEQ ID NO: 1589). The cells are samples at 0, 12, 24, and 48 hours after transfection. After sampling the cells, the cells are lysed and protein samples are prepared of the lysed cells. The protein samples are ran on a SDS-PAGE gel and transferred to a nitrocellulose blot. Protein levels are determined by a Western blot with a primary antibody directed to SLC34A2. Densitometry is used to determine the protein levels of SLC34A2. SLC34A2 protein levels decrease after transfection with the engineered guide RNA.

### Example 15

### Compositions for the Treatment of Facioscapulohumeral Muscular Dystrophy (FSHD)

This example describes a vector for treatment of FSHD. A subject is diagnosed with FSHD, which is caused misexpression of the DUX4 gene. The subject is prescribed a dosing regimen of a pharmaceutical composition. The pharmaceutical composition comprises a vector comprising a engineered guide RNA described herein (e.g., SEQ ID NOs: 2-1589) that is directed to mutate a region in the polyA signal sequence (ATTAAA) of DUX4-FL. The pharmaceutical composition is administered systemically to the subject by intravenous administration in an effective amount to treat the FSHD disease.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

What is claimed is:

1. A composition comprising an engineered guide RNA or an engineered polynucleotide encoding the engineered guide RNA, wherein:
a) the engineered guide RNA, upon hybridization to a sequence of a DUX4 target RNA, forms a guide-target RNA scaffold with the sequence of the DUX4 target RNA;
b) formation of the guide-target RNA scaffold substantially forms one or more structural features selected from the group consisting of: a bulge, an internal loop, a hairpin, and a mismatch formed by a base in the engineered guide RNA to a G, a C, or a U in the DUX4 target RNA; and
c) the structural feature is not present within the engineered guide RNA prior to the hybridization of the engineered guide RNA to the DUX4 target RNA; and
d) upon hybridization of the engineered guide RNA to the sequence of the DUX4 target RNA, the engineered guide RNA facilitates RNA editing of one or more target adenosines in the sequence of the DUX4 target RNA by an RNA editing entity.

2. The composition of claim 1, wherein the sequence of the DUX4 target RNA comprises a translation initiation site, a polyA signal sequence, a splice site, or any combination thereof.

3. The composition of claim 2, wherein the sequence of the DUX4 target RNA comprises the polyA signal sequence.

4. The composition of claim 1, wherein the one or more features further comprises a mismatch formed by a base in the engineered guide RNA to an A in the DUX4 target RNA.

5. The composition of claim 1, wherein the DUX4 is DUX4-FL.

6. The composition of claim 5, wherein the sequence of the DUX4 target RNA comprises the polyA signal sequence, wherein the polyA signal sequence is in DUX4-FL.

7. The composition of claim 6, wherein the polyA signal sequence comprises ATTAAA.

8. The composition of claim 7, wherein any A of the ATTAAA polyA signal sequence is the target adenosine.

9. The composition of any one of claims 5-8, wherein position 0 of ATTAAA is the target adenosine, wherein position 0 is the first A of ATTAAA at the 5′ end.

10. The composition of claim 9, wherein the one or more structural features comprises:
a first 6/6 symmetric internal loop at a position selected from the group consisting of: −3, −4, −5, −6, −7, −8, −9, −10, and −11, relative to position 0 of ATTAAA.

11. The composition of claim 10, wherein the first 6/6 symmetric internal loop is at position −5 relative to position 0.

12. The composition of claim 11, wherein the one or more structural features further comprises a second 6/6 symmetric internal loop at position 33 relative to position 0.

13. The composition of claim 12, wherein the engineered guide RNA comprises at least about: 80%, 85%, 90%, 92%, 95%, 97%, or 99% sequence identity to SEQ ID NO: 1054.

14. The composition of claim 13, wherein the engineered guide RNA comprises SEQ ID NO: 1054.

15. The composition of claim 10, wherein the first 6/6 symmetric internal loop is at position −6 relative to position 0.

16. The composition of claim 15, wherein the one or more structural features further comprises at least one structural feature selected from the group consisting of: an A/C mismatch at position 3 relative to position 0, a second 6/6 symmetric internal loop at position 42 relative to position 0, and a combination thereof.

17. The composition of claim 16, wherein the engineered guide RNA comprises at least about: 80%, 85%, 90%, 92%, 95%, 97%, or 99% sequence identity to SEQ ID NO: 977.

18. The composition of claim 17, wherein the engineered guide RNA comprises SEQ ID NO: 977.

19. The composition of claim 15, wherein the one or more structural features further comprises at least one structural feature selected from the group consisting of: an A/C mismatch at position 0, a second 6/6 symmetric internal loop at position 33 relative to position 0, and a combination thereof.

20. The composition of claim 19, wherein the engineered guide RNA comprises at least about: 80%, 85%, 90%, 92%, 95%, 97%, or 99% sequence identity to SEQ ID NO: 934.

21. The composition of claim 20, wherein the engineered guide RNA comprises SEQ ID NO: 934.

22. The composition of claim 15, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof.

23. The composition of claim 22, wherein the engineered guide RNA comprises at least about: 80%, 85%, 90%, 92%, 95%, 97%, or 99% sequence identity to SEQ ID NO: 1575.

24. The composition of claim 23, wherein the engineered guide RNA comprises SEQ ID NO: 1575.

25. The composition of claim 15, wherein the one or more structural features further comprises at least one structural feature selected from the group consisting of: an A/C mismatch at position 3 relative to position 0, a second 6/6 symmetric internal loop at position 33 relative to position 0, a 5/5 internal loop at position 47 relative to position 0, a 5/5 internal loop at position 60 relative to position 0, a 5/5 internal loop at position 73 relative to position 0, and any combination thereof.

26. The composition of claim 25, wherein the engineered guide RNA comprises at least about: 80%, 85%, 90%, 92%, 95%, 97%, or 99% sequence identity to SEQ ID NO: 1573.

27. The composition of claim 26, wherein the engineered guide RNA comprises SEQ ID NO: 1573.

28. The composition of claim 15, wherein the one or more structural features further comprises at least one structural feature selected from the group consisting of: an A/C mismatch at position 3 relative to position 0, a second 6/6 symmetric internal loop at position 33 relative to position 0, a 5/5 internal loop at position 45 relative to position 0, a 5/5 internal loop at position 56 relative to position 0, a 5/5 internal loop at position 67 relative to position 0, and any combination thereof.

29. The composition of claim 28, wherein the engineered guide RNA comprises at least about: 80%, 85%, 90%, 92%, 95%, 97%, or 99% sequence identity to SEQ ID NO: 1569.

30. The composition of claim 29, wherein the engineered guide RNA comprises SEQ ID NO: 1569.

31. The composition of claim 15, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof.

32. The composition of claim 31, wherein the engineered guide RNA comprises at least about: 80%, 85%, 90%, 92%, 95%, 97%, or 99% sequence identity to SEQ ID NO: 1567.

33. The composition of claim 32, wherein the engineered guide RNA comprises SEQ ID NO: 1567.

34. The composition of claim 15, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof.

35. The composition of claim 34, wherein the engineered guide RNA comprises at least about: 80%, 85%, 90%, 92%, 95%, 97%, or 99% sequence identity to SEQ ID NO: 1588.

36. The composition of claim 35, wherein the engineered guide RNA comprises SEQ ID NO: 1588.

37. The composition of claim 10, wherein the first 6/6 symmetric internal loop is at position −9 relative to position 0.

38. The composition of claim 37, wherein the one or more structural features further comprises at least one structural feature selected from the group consisting of: an A/C mismatch at position 0, a second 6/6 symmetric internal loop at position 40 relative to position 0, and a combination thereof.

39. The composition of claim 38, wherein the engineered guide RNA comprises at least about: 80%, 85%, 90%, 92%, 95%, 97%, or 99% sequence identity to SEQ ID NO: 593.

40. The composition of claim 39, wherein the engineered guide RNA comprises SEQ ID NO: 593.

41. The composition of any one of claims 5-8, wherein position 3 of ATTAAA is the target adenosine, wherein position 3 is the second A of ATTAAA from the 5′ end.

42. The composition of claim 41, wherein the one or more structural features comprises: a first 6/6 symmetric internal loop at a position selected from the group consisting of: 22, 21, 20, −2, −4, −5, −6, −7, −8, −9, and −10 relative to position 0 of ATTAAA.

43. The composition of claim 42, wherein the first 6/6 symmetric internal loop is at position 20 relative to position 0.

44. The composition of claim 43, wherein the one or more structural features further comprises an A/C mismatch at position 3 relative to position 0.

45. The composition of claim 44, wherein the engineered guide RNA comprises at least about: 80%, 85%, 90%, 92%, 95%, 97%, or 99% sequence identity to SEQ ID NO: 8.

46. The composition of claim 45, wherein the engineered guide RNA comprises SEQ ID NO: 8.

47. The composition of claim 42, wherein the first 6/6 symmetric internal loop is at position −5 relative to position 0.

48. The composition of claim 47, wherein the one or more structural features further comprises a second 6/6 symmetric internal loop at position 33 relative to position 0.

49. The composition of claim 48, wherein the engineered guide RNA comprises at least about: 80%, 85%, 90%, 92%, 95%, 97%, or 99% sequence identity to SEQ ID NO: 1054.

50. The composition of claim 49, wherein the engineered guide RNA comprises SEQ ID NO: 1054.

51. The composition of claim 42, wherein the first 6/6 symmetric internal loop is at position −6 relative to position 0.

52. The composition of claim 51, wherein the one or more structural features further comprises at least one structural feature selected from the group consisting of: an A/C mismatch at position 3 relative to position 0, a second 6/6 symmetric internal loop at position 42 relative to position 0, and a combination thereof.

53. The composition of claim 52, wherein the engineered guide RNA comprises at least about: 80%, 85%, 90%, 92%, 95%, 97%, or 99% sequence identity to SEQ ID NO: 977.

54. The composition of claim 53, wherein the engineered guide RNA comprises SEQ ID NO: 977.

55. The composition of claim 51, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof.

56. The composition of claim 55, wherein the engineered guide RNA comprises at least about: 80%, 85%, 90%, 92%, 95%, 97%, or 99% sequence identity to SEQ ID NO: 1569.

57. The composition of claim 56, wherein the engineered guide RNA comprises SEQ ID NO: 1569.

58. The composition of claim 51, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof.

59. The composition of claim 58, wherein the engineered guide RNA comprises at least about: 80%, 85%, 90%, 92%, 95%, 97%, or 99% sequence identity to SEQ ID NO: 1567.

60. The composition of claim 59, wherein the engineered guide RNA comprises SEQ ID NO: 1567.

61. The composition of claim 51, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof.

62. The composition of claim 61, wherein the engineered guide RNA comprises at least about: 80%, 85%, 90%, 92%, 95%, 97%, or 99% sequence identity to SEQ ID NO: 1573.

63. The composition of claim 62, wherein the engineered guide RNA comprises SEQ ID NO: 1573.

64. The composition of claim 51, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof.

65. The composition of claim 64, wherein the engineered guide RNA comprises at least about: 80%, 85%, 90%, 92%, 95%, 97%, or 99% sequence identity to SEQ ID NO: 1588.

66. The composition of claim 65, wherein the engineered guide RNA comprises SEQ ID NO: 1588.

67. The composition of claim 51, wherein the one or more structural features further comprises at least one structural feature selected from the group consisting of: A/C mismatch at position 3, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof.

68. The composition of claim 67, wherein the engineered guide RNA comprises at least about: 80%, 85%, 90%, 92%, 95%, 97%, or 99% sequence identity to SEQ ID NO: 1575.

69. The composition of claim 68, wherein the engineered guide RNA comprises SEQ ID NO: 1575.

70. The composition of claim 42, wherein the first 6/6 symmetric internal loop is at position −9 relative to position 0.

71. The composition of claim 70, wherein the one or more structural features further comprises at least one structural feature selected from the group consisting of: an A/C mismatch at position 0, a second 6/6 symmetric internal loop at position 40 relative to position 0, and a combination thereof.

72. The composition of claim 71, wherein the engineered guide RNA comprises at least about: 80%, 85%, 90%, 92%, 95%, 97%, or 99% sequence identity to SEQ ID NO: 593.

73. The composition of claim 72, wherein the engineered guide RNA comprises SEQ ID NO: 593.

74. The composition of claim 41, wherein the one or more structural features comprises: a first 2/2 symmetric bulge at a position selected from the group consisting of: −3, −5, and −7 relative to position 0 of ATTAAA.

75. The composition of claim 74, wherein the first 2/2 symmetric bulge is at position −5 relative to position 0.

76. The composition of claim 75, wherein the one or more structural features further comprises at least one structural feature selected from the group consisting of: a 2/2 symmetric bulge at position 26 relative to position 0, a 2/2 symmetric bulge at position 42 relative to position 0, a 2/2 symmetric bulge at position 58 relative to position 0, a 2/2 symmetric bulge at position 74 relative to position 0, and any combination thereof.

77. The composition of claim 76, wherein the engineered guide RNA comprises at least about: 80%, 85%, 90%, 92%, 95%, 97%, or 99% sequence identity to SEQ ID NO: 1545.

78. The composition of claim 77, wherein the engineered guide RNA comprises SEQ ID NO: 1545.

79. The composition of any one of claims 5-8, wherein position 4 of ATTAAA is the target adenosine, wherein position 4 is the third A of ATTAAA from the 5′ end.

80. The composition of claim 79, wherein the one or more structural features comprises:
a first 6/6 symmetric internal loop at a position selected from the group consisting of: 33, −1, −2, −3, −4, −5, −6, −7, −8, −9, −11, and −12 relative to position 0 of ATTAAA.

81. The composition of claim 80, wherein the first 6/6 symmetric internal loop is at position −1 relative to position 0.

82. The composition of claim 81, wherein the one or more structural features further comprises at least one structural feature selected from the group consisting of: an A/C mismatch at position 4 relative to position 0, a second 6/6 symmetric internal loop at position 32 relative to position 0, and a combination thereof.

83. The composition of claim 82, wherein the engineered guide RNA comprises at least about: 80%, 85%, 90%, 92%, 95%, 97%, or 99% sequence identity to SEQ ID NO: 1463.

84. The composition of claim 83, wherein the engineered guide RNA comprises SEQ ID NO: 1463.

85. The composition of claim 80, wherein the first 6/6 symmetric internal loop is at position −3 relative to position 0.

86. The composition of claim 85, wherein the one or more structural features further comprises at least one structural feature selected from the group consisting of: an A/C mismatch at position 4 relative to position 0, a second 6/6 symmetric internal loop at position 36 relative to position 0, and a combination thereof.

87. The composition of claim 86, wherein the engineered guide RNA comprises at least about: 80%, 85%, 90%, 92%, 95%, 97%, or 99% sequence identity to SEQ ID NO: 1294.

88. The composition of claim 87, wherein the engineered guide RNA comprises SEQ ID NO: 1294.

89. The composition of claim 80, wherein the first 6/6 symmetric internal loop is at position −5 relative to position 0.

90. The composition of claim 89, wherein the one or more structural features further comprises a second 6/6 symmetric internal loop at position 33 relative to position 0.

91. The composition of claim 90, wherein the engineered guide RNA comprises at least about: 80%, 85%, 90%, 92%, 95%, 97%, or 99% sequence identity to SEQ ID NO: 1054.

92. The composition of claim 91, wherein the engineered guide RNA comprises SEQ ID NO: 1054.

93. The composition of claim 80, wherein the first 6/6 symmetric internal loop is at position −6 relative to position 0.

94. The composition of claim 93, wherein the one or more structural features further comprises at least one structural feature selected from the group consisting of: an A/C mismatch at position 0, a second 6/6 symmetric internal loop at position 33 relative to position 0, and a combination thereof.

95. The composition of claim 94, wherein the engineered guide RNA comprises at least about: 80%, 85%, 90%, 92%, 95%, 97%, or 99% sequence identity to SEQ ID NO: 934.

96. The composition of claim 95, wherein the engineered guide RNA comprises SEQ ID NO: 934.

97. The composition of claim 93, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof.

98. The composition of claim 97, wherein the engineered guide RNA comprises at least about: 80%, 85%, 90%, 92%, 95%, 97%, or 99% sequence identity to SEQ ID NO: 1573.

99. The composition of claim 98, wherein the engineered guide RNA comprises SEQ ID NO: 1573.

100. The composition of claim 93, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof.

101. The composition of claim 100, wherein the engineered guide RNA comprises at least about: 80%, 85%, 90%, 92%, 95%, 97%, or 99% sequence identity to SEQ ID NO: 1575.

102. The composition of claim 101, wherein the engineered guide RNA comprises SEQ ID NO: 1575.

103. The composition of claim 93, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof.

104. The composition of claim 103, wherein the engineered guide RNA comprises at least about: 80%, 85%, 90%, 92%, 95%, 97%, or 99% sequence identity to SEQ ID NO: 1567.

105. The composition of claim 104, wherein the engineered guide RNA comprises SEQ ID NO: 1567.

106. The composition of claim 93, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof.

107. The composition of claim 106, wherein the engineered guide RNA comprises at least about: 80%, 85%, 90%, 92%, 95%, 97%, or 99% sequence identity to SEQ ID NO: 1569.

108. The composition of claim 107, wherein the engineered guide RNA comprises SEQ ID NO: 1569.

109. The composition of claim 93, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof.

110. The composition of claim 109, wherein the engineered guide RNA comprises at least about: 80%, 85%, 90%, 92%, 95%, 97%, or 99% sequence identity to SEQ ID NO: 1588.

111. The composition of claim 110, wherein the engineered guide RNA comprises SEQ ID NO: 1588.

112. The composition of claim 80, wherein the first 6/6 symmetric internal loop is at position −9 relative to position 0.

113. The composition of claim 112, wherein the one or more structural features further comprises at least one structural feature selected from the group consisting of: an A/C mismatch at position 0, a second 6/6 symmetric internal loop at position 40 relative to position 0, and a combination thereof.

114. The composition of claim 113, wherein the engineered guide RNA comprises at least about: 80%, 85%, 90%, 92%, 95%, 97%, or 99% sequence identity to SEQ ID NO: 593.

115. The composition of claim 114, wherein the engineered guide RNA comprises SEQ ID NO: 593.

116. The composition of any one of claims 5-8, wherein position 5 of ATTAAA is the target adenosine, wherein position 5 is the forth A of ATTAAA from the 5′ end.

117. The composition of claim 81, wherein the one or more structural features comprises: a first 6/6 symmetric internal loop at a position selected from the group consisting of: 33, 23, −1, −2, −3, −4, −5, −6, −7, −8, −9, −10, and −12 relative to position 0 of ATTAAA.

118. The composition of claim 117, wherein the first 6/6 symmetric internal loop is at position −1 relative to position 0.

119. The composition of claim 118, wherein the one or more structural features further comprises at least one structural feature selected from the group consisting of: an A/C mismatch at position 4 relative to position 0, a second 6/6 symmetric internal loop at position 32 relative to position 0, and a combination thereof.

120. The composition of claim 119, wherein the engineered guide RNA comprises at least about: 80%, 85%, 90%, 92%, 95%, 97%, or 99% sequence identity to SEQ ID NO: 1463.

121. The composition of claim 120, wherein the engineered guide RNA comprises SEQ ID NO: 1463.

122. The composition of claim 117, wherein the first 6/6 symmetric internal loop is at position −5 relative to position 0.

123. The composition of claim 122, wherein the one or more structural features further comprises a second 6/6 symmetric internal loop at position 33 relative to position 0.

124. The composition of claim 123, wherein the engineered guide RNA comprises at least about: 80%, 85%, 90%, 92%, 95%, 97%, or 99% sequence identity to SEQ ID NO: 1054.

125. The composition of claim 124, wherein the engineered guide RNA comprises SEQ ID NO: 1054.

126. The composition of claim 117, wherein the first 6/6 symmetric internal loop is at position −6 relative to position 0.

127. The composition of claim 126, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 49 relative to position 0, a 3/3 symmetric bulge at position 62 relative to position 0, a 3/3 symmetric bulge at position 75 relative to position 0, and any combination thereof.

128. The composition of claim 127, wherein the engineered guide RNA comprises at least about: 80%, 85%, 90%, 92%, 95%, 97%, or 99% sequence identity to SEQ ID NO: 1575.

129. The composition of claim 128, wherein the engineered guide RNA comprises SEQ ID NO: 1575.

130. The composition of claim 126, wherein the one or more structural features further comprises at least one structural feature selected from the group consisting of: an A/C mismatch at position 3 relative to position 0, a second 6/6 symmetric internal loop at position 33 relative to position 0, a 3/3 symmetric bulge at position 45 relative to position 0, a 3/3 symmetric bulge at position 54 relative to position 0, a 3/3 symmetric bulge at position 63 relative to position 0, a 3/3 symmetric bulge at position 72 relative to position 0, and any combination thereof.

131. The composition of claim 130, wherein the engineered guide RNA comprises at least about: 80%, 85%, 90%, 92%, 95%, 97%, or 99% sequence identity to SEQ ID NO: 1567.

132. The composition of claim 131, wherein the engineered guide RNA comprises SEQ ID NO: 1567.

133. The composition of claim 126, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 47 relative to position 0, a 5/5 symmetric internal loop at position 60 relative to position 0, a 5/5 symmetric internal loop at position 73 relative to position 0, and any combination thereof.

134. The composition of claim 133, wherein the engineered guide RNA comprises at least about: 80%, 85%, 90%, 92%, 95%, 97%, or 99% sequence identity to SEQ ID NO: 1573.

135. The composition of claim 134, wherein the engineered guide RNA comprises SEQ ID NO: 1573.

136. The composition of claim 126, wherein the one or more structural features further comprises at least one structural feature selected from the group consisting of: an A/C mismatch at position 3 relative to position 0, a second 6/6 symmetric internal loop at position 33 relative to position 0, a 5/5 symmetric internal loop at position 45 relative to position 0, a 5/5 symmetric internal loop at position 56 relative to position 0, a 5/5 symmetric internal loop at position 67 relative to position 0, and any combination thereof.

137. The composition of claim 136, wherein the engineered guide RNA comprises at least about: 80%, 85%, 90%, 92%, 95%, 97%, or 99% sequence identity to SEQ ID NO: 1569.

138. The composition of claim 137, wherein the engineered guide RNA comprises SEQ ID NO: 1569.

139. The composition of claim 126, wherein the one or more structural features further comprises at least one structural feature selected from the group consisting of: an A/C mismatch at position 3 relative to position 0, a second 6/6 symmetric internal loop at position 33 relative to position 0, a 4/4 symmetric bulge at position 55 relative to position 0, a 4/4 symmetric bulge at position 75 relative to position 0, and any combination thereof.

140. The composition of claim 139, wherein the engineered guide RNA comprises at least about: 80%, 85%, 90%, 92%, 95%, 97%, or 99% sequence identity to SEQ ID NO: 1588.

141. The composition of claim 140, wherein the engineered guide RNA comprises SEQ ID NO: 1588.

142. The composition of any one of claims 9-141, further comprising editing at any A of ATTAAA.

143. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

144. The composition of claim 143, wherein the engineered guide RNA has at least 80%, 85%, 90%, 92%, 95%, 97%, or 99% sequence identity to a guide RNA comprising SEQ ID NO: 8.

145. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

146. The composition of claim 145, wherein the engineered guide RNA has at least 80%, 85%, 90%, 92%, 95%, 97%, or 99% sequence identity to a guide RNA comprising SEQ ID NO: 593.

147. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

148. The composition of claim 147, wherein the engineered guide RNA has at least 80%, 85%, 90%, 92%, 95%, 97%, or 99% sequence identity to a guide RNA comprising SEQ ID NO: 934.

149. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

150. The composition of claim 149, wherein the engineered guide RNA has at least 80%, 85%, 90%, 92%, 95%, 97%, or 99% sequence identity to a guide RNA comprising SEQ ID NO: 977.

151. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

152. The composition of claim 151, wherein the engineered guide RNA has at least 80%, 85%, 90%, 92%, 95%, 97%, or 99% sequence identity to a guide RNA comprising SEQ ID NO: 1054.

153. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

154. The composition of claim 153, wherein the engineered guide RNA has at least 80%, 85%, 90%, 92%, 95%, 97%, or 99% sequence identity to a guide RNA comprising SEQ ID NO: 1294.

155. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

156. The composition of claim 155, wherein the engineered guide RNA has at least 80%, 85%, 90%, 92%, 95%, 97%, or 99% sequence identity to a guide RNA comprising SEQ ID NO: 1463.

157. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

158. The composition of claim 157, wherein the engineered guide RNA has at least 80%, 85%, 90%, 92%, 95%, 97%, or 99% sequence identity to a guide RNA comprising SEQ ID NO: 1545.

159. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

160. The composition of claim 159, wherein the engineered guide RNA has at least 80%, 85%, 90%, 92%, 95%, 97%, or 99% sequence identity to a guide RNA comprising SEQ ID NO: 1567.

161. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

162. The composition of claim 161, wherein the engineered guide RNA has at least 80%, 85%, 90%, 92%, 95%, 97%, or 99% sequence identity to a guide RNA comprising SEQ ID NO: 1569.

163. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

164. The composition of claim 163, wherein the engineered guide RNA has at least 80%, 85%, 90%, 92%, 95%, 97%, or 99% sequence identity to a guide RNA comprising SEQ ID NO: 1573.

165. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

166. The composition of claim 165, wherein the engineered guide RNA has at least 80%, 85%, 90%, 92%, 95%, 97%, or 99% sequence identity to a guide RNA comprising SEQ ID NO: 1575.

167. The composition of claim 6, wherein the one or more structural features comprise a 1 nucleotide mismatch formed 3 nucleotides downstream (3′) from the target A, and a 6 nucleotide internal symmetric loop formed 20 nucleotides downstream (3′) from the target A.

168. The composition of claim 167, wherein the engineered guide RNA has at least 80%, 85%, 90%, 92%, 95%, 97%, or 99% sequence identity to a guide RNA comprising SEQ ID NO: 1588.

169. The composition of any one of claims 5-7, wherein the one or more structural features comprise:
a) a first 6/6 symmetric internal loop, and
b) at least one additional structural feature selected from the group consisting of:
a second 6/6 symmetric internal loop, a 5/5 symmetric internal loop, a 4/4 symmetric bulge, a 3/3 symmetric bulge, and a 2/2 symmetric bulge.

170. The composition of claim 169, wherein the guide-target RNA scaffold further comprises an A/C mismatch, wherein the cytosine of the A/C mismatch is present in the engineered guide RNA opposite the one or more target adenosines; and wherein the one or more structural features comprise:
a) the first 6/6 symmetric internal loop positioned from position −4 to −8, relative to the A/C mismatch;
b) the second 6/6 symmetric internal loop positioned from position+31 to +35, relative to the A/C mismatch.

171. The composition of claim 170, wherein the guide-target RNA scaffold further comprises an A/C mismatch, wherein the cytosine of the A/C mismatch is present in the engineered guide RNA opposite the one or more target adenosines; and wherein the one or more structural features comprise:
a) the first 6/6 symmetric internal loop at position −6, relative to the A/C mismatch;
b) the second 6/6 symmetric internal loop at position+33, relative to the A/C mismatch.

172. The composition of claim 170 or 171, wherein the first 6/6 symmetric internal loop comprises the sequence GGAACU on the engineered guide RNA side, and the sequence UUCAGA on the target RNA side.

173. The composition of claim 170 or 171, wherein the second 6/6 symmetric internal loop comprises the sequence CUGACC on the engineered guide RNA side, and the sequence AGAUUU on the target RNA side.

174. The composition of any one of claims 5-7, wherein the one or more structural features comprise a first 6/6 symmetric internal loop and a second 6/6 symmetric internal loop and wherein each A in the target RNA is base paired to a U in the engineered guide RNA.

175. The composition of claim 1, wherein the one or more structural features comprises the bulge, wherein the bulge is a symmetric bulge.

176. The composition of claim 1, wherein the one or more structural features comprises the bulge, wherein the bulge is an asymmetric bulge.

177. The composition of claim 1, wherein the one or more structural features comprises the internal loop, wherein the internal loop is a symmetric internal loop.

178. The composition of claim 1, wherein the one or more structural features comprises the internal loop, wherein the internal loop is an asymmetric internal loop.

179. The composition of claim 1, wherein the one or more structural features comprises the mismatch formed by a base in the engineered guide RNA to a G, a C, or a U in the DUX4 target RNA.

180. The composition of claim 1, wherein the RNA editing entity comprises ADAR1, ADAR2, ADAR3, or any combination thereof.

181. The composition of claim 1, wherein the RNA editing of one or more target adenosines comprises hyper-editing.

182. The composition of claim 181, wherein the hyper-editing comprises editing of more than one A in the polyA signal sequence of the DUX4 target RNA.

183. The composition of claim 1, wherein the internal loop of the engineered guide RNA comprises any nucleotide in any positional order, wherein the nucleotide in any positional order is not complementary to their positional counterpart in the DUX 4 target RNA.

184. The composition of any one of claims 1-183, wherein the engineered guide RNA or the engineered polynucleotide encoding the engineered guide RNA is circular.

185. The composition of any one of claims 1-184, wherein the engineered guide RNA or the engineered polynucleotide encoding the engineered guide RNA comprises a U7 hairpin sequence, a SmOPT sequence, or a combination thereof and optionally wherein the U7 hairpin sequence comprises SEQ ID NO 1591 or 1593 and wherein the SmOPT sequence comprises SEQ ID NO: 1595.

186. The composition of claim 1, wherein the DUX4 target RNA comprises a pre-mRNA transcript of DUX4.

187. The composition of claim 186, wherein at least 40%, at least 50%, at least 60%, at least 70%, at least 80%, at least 85%, at least 90%, at least 95%, or at least 99% of the pre-mRNA transcripts of DUX4 have at least one edit in the polyA signal sequence.

188. The composition of claim 187, wherein at least 80% of the pre-mRNA transcripts of DUX4 have at least one edit in the polyA signal sequence.

189. The composition of any one of claims 1-188, wherein the editing of one or more adenosines facilitates a mRNA knockdown.

190. The composition of claim 189, wherein the mRNA knockdown comprises a knockdown of DUX4 mRNA.

191. The composition of any one of claims 189 or 190, wherein the mRNA knockdown comprises a reduction of at least 40%, at least 50%, at least 60%, at least 70%, at least 80%, at least 85%, at least 90%, at least 95%, or at least 99% of a mRNA level after RNA editing as compared to a mRNA level before RNA editing.

192. The composition of claim 191, wherein the mRNA knockdown is at least 50% of the mRNA level as compared to the mRNA level before RNA editing.

193. The composition of claim 191, wherein the mRNA knockdown is at least 70% of the mRNA level as compared to the mRNA level before RNA editing.

194. The composition of any one of claims 1-193, wherein the editing of one or more adenosines facilitates a protein knockdown.

195. The composition of claim 194, wherein the protein knockdown comprises a knockdown of DUX4.

196. The composition of claim 194 or 195, wherein the protein knockdown comprises a knockdown of a protein downstream of DUX4, wherein the protein downstream of DUX4 comprises SLC34A2, LEUTX, ZSCAN4, PRAMEF12, TRIM43, DEFB103, or MBD3L2, or any combination thereof.

197. The composition of any one of claims 194-196, wherein the protein knockdown comprises a reduction of at least 40%, at least 50%, at least 60%, at least 70%, at least 80%, at least 85%, at least 90%, at least 95%, or at least 99% of the protein level after RNA editing as compared to the protein level before RNA editing.

198. The composition of any one of claims 194-196, wherein the protein knockdown comprises a reduction of at least 40%, at least 50%, at least 60%, at least 70%, at least 80%, at least 85%, at least 90%, at least 95%, or at least 99% of the protein level in an ADAR expressing cell as compared to a cell comprising an nonfunctional ADAR gene.

199. The composition of any one of claims 194-198, wherein the protein knockdown comprises ADAR-dependent protein knockdown.

200. The composition of claim 199, wherein the ADAR-dependent protein knockdown comprises a reduction of at least 50%, at least 60%, at least 70%, at least 80%, at least 85%, at least 90%, at least 95%, or at least 99% of the protein level as compared to the protein level before RNA editing.

201. The composition of any one of claims 1-200, wherein the engineered guide RNA is an in vitro transcribed (IVT) engineered guide RNA.

202. The composition of any one of claims 1-200, comprising the engineered polynucleotide.

203. The composition of claim 202, wherein the engineered polynucleotide is comprised in or on a vector.

204. The composition of claim 203, wherein the vector is a viral vector, and wherein the engineered polynucleotide is encapsidated in the viral vector.

205. The composition of claim 204, wherein the viral vector is an adeno-associated viral (AAV) vector or a derivative thereof.

206. The composition of claim 203, wherein the vector is a non-viral vector.

207. The composition of claim 206, wherein the non-viral vector is a lipid nanoparticle (LNP), a liposome, or a polymer nanoparticle.

208. The composition of claim 202, wherein the engineered polynucleotide is a DNA polynucleotide encoding the engineered guide RNA.

209. The composition of claim 1, wherein the engineered guide RNA comprises at least 80%, 85%, 90%, 92%, 95%, 97%, or 99% sequence identity to any one of SEQ ID NO: 2-SEQ ID NO: 1589.

210. The composition of claim 1, wherein the engineered guide RNA comprises a sequence of any one of SEQ ID NO: 2-SEQ ID NO: 1589.

211. A pharmaceutical composition comprising:
a) the composition of any one of claims 1-210; and
b) a pharmaceutically acceptable: excipient, carrier, or diluent.

212. A method of treating a disease or a condition in a subject in need thereof, the method comprising administering to the subject a therapeutically effective amount of the composition of any one of claims 1-210 or the pharmaceutical composition of claim 211.

213. The method of claim 212, wherein the disease or condition comprises facioscapulohumeral muscular dystrophy (FSHD).

214. The method of claim 213, wherein FSHD comprises Type I FSHD.

215. The method of claim 213, wherein FSHD comprises Type II FSHD.

216. The method of any one of claims 212-215, wherein the administering comprises parenteral administration, intravenous administration, subcutaneous administration, intrathecal administration, intraperitoneal administration, intramuscular administration, intravascular administration, infusion administration, topical administration, oral administration, inhalation administration, intraduodenal administration, rectal administration, or a combination thereof.

217. The method of claim 216, comprising the administering, wherein the administration is oral administration.

218. The method of any one of claims 212-217, wherein the administering comprises systemic administration.

219. A method of editing a DUX4 RNA the method comprising contacting the DUX4 RNA with any one of the compositions of claims 1-210 and an RNA editing entity, thereby editing the DUX4 RNA.

220. The method of claim 219, wherein the editing comprises editing at any A position of a polyA tail of the DUX4 RNA.

221. The method of claim 219, wherein the DUX4 RNA comprises a pre-mRNA transcript of DUX4.

222. The method of claim 221, wherein at least 40%, at least 50%, at least 60%, at least 70%, at least 80%, at least 85%, at least 90%, at least 95%, or at least 99% of the pre-mRNA transcripts of DUX4 have at least one edit in the polyA signal sequence.

223. The method of claim 219, wherein the editing of DUX4 RNA facilitates a protein knockdown.

224. The method of claim 223, wherein the protein knockdown comprises a knockdown of DUX4.

225. The composition of any one of claims 1-210 or the pharmaceutical composition of claim 211 for use as a medicament.

226. The composition of any one of claims 1-210 or the pharmaceutical composition of claim 211 for use in the treatment of facioscapulohumeral muscular dystrophy (FSHD).

227. The composition of claim 226, wherein FSHD comprises Type I FSHD.

228. The composition of claim 226, wherein FSHD comprises Type II FSHD.
